(19) United States
(12) Patent Application Publication
(10) Pub. No.: US 2025/0248305 A1
(43) Pub. Date: Jul. 31, 2025

(54) ORGANIC ELECTROLUMINESCENT COMPOUND, A PLURALITY OF HOST MATERIALS, AND ORGANIC ELECTROLUMINESCENT DEVICE COMPRISING THE SAME

(71) Applicant: DuPont Specialty Materials Korea Ltd., Chungcheongnam-do (KR)

(72) Inventors: So-Young JUNG, Gyeonggi-do (KR); Sang-Hee CHO, Gyeonggi-do (KR); So-Mi PARK, Gyeonggi-do (KR); Doo-Hyeon MOON, Gyeonggi-do (KR); Tae-Jun HAN, Gyeonggi-do (KR); Su-Hyun LEE, Gyeonggi-do (KR); Hyo-Jung LEE, Gyeonggi-do (KR); Mi-Ran SEO, Gyeonggi-do (KR); Bitnari KIM, Gyeonggi-do (KR); Du-Yong PARK, Gyeonggi-do (KR); YeJin JEON, Gyeonggi-do (KR); Hyun-Ju KANG, Gyeonggi-do (KR)

(21) Appl. No.: 19/033,643

(22) Filed: Jan. 22, 2025

(30) Foreign Application Priority Data
Jan. 31, 2024 (KR) 10-2024-0014649
Dec. 24, 2024 (KR) 10-2024-0195577

(51) Int. Cl. H10K 85/60; H10K 85/40; H10K 101/00

(52) U.S. Cl. CPC H10K 85/6576; H10K 85/40; H10K 85/622; H10K 85/626; H10K 85/633; H10K 85/654; H10K 85/6572; H10K 85/6574; H10K 2101/90

(57) ABSTRACT

The present disclosure relates to an organic electroluminescent compound, a plurality of host materials, and an organic electroluminescent device comprising the same. By comprising a compound or a host material according to the present disclosure as host materials, an organic electroluminescent device having high luminous efficiency and/or long lifespan characteristics can be provided.

### TECHNICAL FIELD

The present disclosure relates to an organic electroluminescent compound, a plurality of host materials, and organic electroluminescent device comprising the same.

### BACKGROUND ART

An electroluminescent device (EL device) is a self-light-emitting display device which has advantages in that it provides a wider viewing angle, a greater contrast ratio, and a faster response time. An organic EL device was first developed by Eastman Kodak in 1987 by using small aromatic diamine molecules and aluminum complexes as materials for forming a light-emitting layer (Appl. Phys. Lett. 51, 913, 1987).

The light-emitting material of an organic electroluminescent device is the most important factor determining the luminous efficiency of the device. Light-emitting materials are required to have the following features: high quantum efficiency, high degrees of movement for electrons and holes, and uniformity and stability of the formed light-emitting material layer. Light-emitting materials are classified according to function as host materials and dopant materials. In order to improve color purity, luminous efficiency, and stability, a mixture of host and dopant can be used. The desirable properties of the host material, which acts as a solid-state solvent and energy carrier, are that it should have high purity and an appropriate molecular weight to enable vacuum deposition. In addition, it should have a high glass transition temperature and a high thermal decomposition temperature to ensure thermal stability, high electrochemical stability is required for long lifespan, it should be easy to form an amorphous thin film, and it is desirable that it has good adhesion to materials of adjacent other layers without undergoing interlayer migration. When using such a dopant/host material system, the selection of the host material is important because it has a great influence on the efficiency and lifespan of the light-emitting device.

Although various compounds have been known as host materials to date, there has been a demand for new materials due to high driving voltage, low efficiency, and short lifespan in organic electroluminescent devices using conventionally known materials. Therefore, it is necessary to develop a host material that enables the implementation of an organic electroluminescent device with excellent lifespan characteristics even at low-voltage operation and high luminance.

Korean Patent Application Laid-Open No. 10-2017-0022865 A discloses an organic electroluminescent device using phenanthrooxazole and phenanthrothiazole compounds as hosts, but does not specifically disclose an organic electroluminescent device using a plurality of host materials of a specific combination specified herein, and there is still a demand for the development of host materials for improving the performance of OLEDs.

Korean Patent Application Laid-Open No. 10-2014-0057439 A discloses an organic electroluminescent device using a heterocyclic compound as a host or hole transport material, but does not specifically disclose an organic electroluminescent device using a plurality of host materials of a specific combination specified herein, and there is still a demand for the development of host materials for improving the performance of OLEDs.

Korean Patent No. 10-1545774 B1 discloses an organic electronic device comprising a compound having a chrysene moiety in a light-emitting layer, but does not specifically disclose an organic electroluminescent device using a plurality of host materials of a specific combination specified herein, and there is still a demand for the development of host materials for improving the performance of OLEDs.

### DISCLOSURE OF THE INVENTION

### Technical Problem

The object of the present disclosure is, firstly, to provide a plurality of host materials or organic electroluminescent compounds capable of producing an organic electroluminescent device having high luminous efficiency characteristics and/or long lifespan characteristics, and secondly, to provide an organic electroluminescent device comprising the host materials.

### Solution to Problem

As a result of intensive studies to solve the technical problem above, the present inventors found that the aforementioned objective can be achieved by a plurality of host materials comprising a first host material comprising a compound represented by the following Formula 1 and a second host material comprising a compound represented by the following Formula 2, or an organic electroluminescent compound represented by any one of the following Formulas 3-1-1 to 3-4-1, thereby completing the present disclosure.

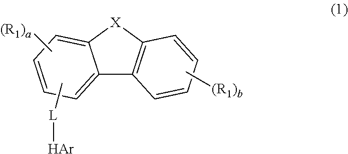

In Formula 1,
X represents O, S, or Se;
HAr represents a substituted or unsubstituted (3- to 30-membered)heteroaryl containing one or more nitrogen atoms;
L represents a single bond, a substituted or unsubstituted (C₆-C₃₀)arylene, or a substituted or unsubstituted (3- to 30-membered)heteroarylene;
R₁ and R₂ each independently represent hydrogen, deuterium, a halogen, a cyano, a substituted or unsubstituted (C₁-C₃₀)alkyl, a substituted or unsubstituted (C₆-C₃₀)aryl, a substituted or unsubstituted (3- to 30-membered)heteroaryl, a substituted or unsubstituted (C₃-C₃₀)cycloalkyl, a substituted or unsubstituted (C₁-C₃₀)alkoxy, a substituted or unsubstituted tri(C₁-C₃₀)alkylsilyl, a substituted or unsubstituted di(C₁-C₃₀)alkyl(C₆-C₃₀)arylsilyl, a substituted or unsubstituted (C₁-C₃₀)alkyldi(C₆-C₃₀)arylsilyl, or a substituted or unsubstituted tri(C₆-C₃₀)arylsilyl; or may be linked to the adjacent substituents to form a ring(s);
provided that at least one of R₁ and R₂ is a substituted or unsubstituted (C₆-C₃₀)aryl or a substituted or unsubstituted (3- to 30-membered)heteroaryl; and
a represents an integer of 1 to 3, b represents an integer of 1 to 4, and when a and b are an integer of 2 or more, each of R₁ and each of R₂ may be the same as or different from each other.

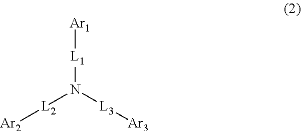

In Formula 2,
L₁ to L₃ each independently represent a single bond, a substituted or unsubstituted (C₆-C₃₀)arylene, or a substituted or unsubstituted (3- to 30-membered)heteroarylene;
Ar₁ represents a substituted or unsubstituted (C₆-C₃₀)aryl, or a substituted or unsubstituted (3- to 30-membered)heteroaryl; and
Ar₂ and Ar₃ each independently represent hydrogen, deuterium, a halogen, a cyano, a substituted or unsubstituted (C₁-C₃₀)alkyl, a substituted or unsubstituted (C₆-C₃₀)aryl, a substituted or unsubstituted (3- to 30-membered)heteroaryl, a substituted or unsubstituted (C₃-C₃₀)cycloalkyl, a substituted or unsubstituted (C₁-C₃₀)alkoxy, a substituted or unsubstituted tri(C₁-C₃₀)alkylsilyl, a substituted or unsubstituted di(C₁-C₃₀)alkyl(C₆-C₃₀)arylsilyl, a substituted or unsubstituted (C₁-C₃₀)alkyldi(C₆-C₃₀)arylsilyl, a substituted or unsubstituted tri(C₆-C₃₀)arylsilyl, a substituted or unsubstituted mono- or di(C₁-C₃₀)alkylamino, a substituted or unsubstituted mono- or di(C₂-C₃₀)alkenylamino, a substituted or unsubstituted (C₁-C₃₀)alkyl(C₂-C₃₀)alkenylamino, a substituted or unsubstituted mono- or di(C₆-C₃₀)arylamino, a substituted or unsubstituted (C₁-C₃₀)alkyl(C₆-C₃₀)arylamino, a substituted or unsubstituted mono- or di(3- to 30-membered)heteroarylamino, a substituted or unsubstituted (C₁-C₃₀)alkyl(3- to 30-membered)heteroarylamino, a substituted or unsubstituted (C₂-C₃₀)alkenyl(C₆-C₃₀)arylamino, a substituted or unsubstituted (C₂-C₃₀)alkenyl(3- to 30-membered)heteroarylamino, a substituted or unsubstituted (C₆-C₃₀)aryl(3- to 30-membered)heteroarylamino, or a substituted or unsubstituted fused ring of (C₃-C₃₀) aliphatic ring and (C₆-C₃₀) aromatic ring; or may be linked to the adjacent substituents to form a ring(s).

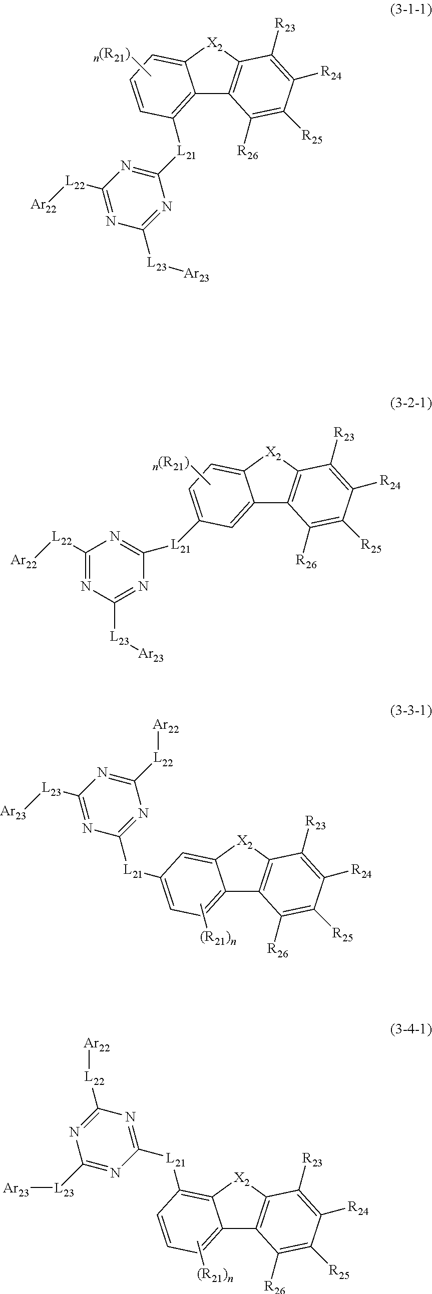

In Formulas 3-1-1 to 3-4-1,
X₂ represents O or S;
L₂₁ and L₂₂ each independently represent a single bond, a substituted or unsubstituted (C₆-C₃₀)arylene, or a substituted or unsubstituted (3- to 30-membered)heteroarylene;
L₂₃ represents phenylene unsubstituted or substituted with deuterium;
Ar₂₂ represents a substituted or unsubstituted (C₆-C₃₀)aryl, or a substituted or unsubstituted (3- to 30-membered)heteroaryl;
Ar₂₃ represents naphthyl substituted with phenyl, which may be further substituted with deuterium;
R₂₃ to R₃₀ each independently represent hydrogen, deuterium, a halogen, a cyano, a substituted or unsubstituted (C₁-C₃₀)alkyl, a substituted or unsubstituted (C₃-C₃₀)cycloalkyl, a substituted or unsubstituted (C₆-C₃₀)aryl, a substituted or unsubstituted (3- to 30-membered)heteroaryl, a substituted or unsubstituted (C₁-C₃₀)alkoxy, a substituted or unsubstituted tri(C₁-C₃₀)alkylsilyl, a substituted or unsubstituted di(C₁-C₃₀)alkyl(C₆-C₃₀)arylsilyl, a substituted or unsubstituted (C₁-C₃₀)alkyldi(C₆-C₃₀)arylsilyl, a substituted or unsubstituted tri(C₆-C₃₀)arylsilyl, a substituted or unsubstituted mono- or di(C₁-C₃₀)alkylamino, a substituted or unsubstituted mono- or di(C₂-C₃₀)alkenylamino, a substituted or unsubstituted (C₁-C₃₀)alkyl(C₂-C₃₀)alkenylamino, a substituted or unsubstituted mono- or di(C₆-C₃₀)arylamino, a substituted or unsubstituted (C₁-C₃₀)alkyl(C₆-C₃₀)arylamino, a substituted or unsubstituted mono- or di(3- to 30-membered)heteroarylamino, a substituted or unsubstituted (C₁-C₃₀)alkyl(3- to 30-membered)heteroarylamino, a substituted or unsubstituted (C₂-C₃₀)alkenyl(C₆-C₃₀)arylamino, a substituted or unsubstituted (C₂-C₃₀)alkenyl(3- to 30-membered)heteroarylamino, a substituted or unsubstituted (C₆-C₃₀)aryl(3- to 30-membered)heteroarylamino, or a substituted or unsubstituted fused ring of (C₃-C₃₀) aliphatic ring and (C₆-C₃₀) aromatic ring;
provided that in each of Formulas 3-1-1 to 3-4-1, at least one of R₂₃ to R₂₆ is a substituted or unsubstituted (C₆-C₃₀)aryl, or a substituted or unsubstituted (3- to 30-membered)heteroaryl.

### Advantageous Effects of Invention

By comprising an organic electroluminescent compound or a plurality of host materials according to the present disclosure, an organic electroluminescent device having high luminous efficiency characteristics and/or long lifespan characteristics can be provided.

### MODE FOR THE INVENTION

Hereinafter, the present disclosure will be described in detail. However, the following description is intended to explain the invention, and is not meant in any way to restrict the scope of the present disclosure.

The present disclosure relates to a plurality of host materials including at least one first host material including a compound represented by Formula 1 and at least one second host material including a compound represented by Formula 2, and an organic electroluminescent device including the host materials. In addition, the present disclosure relates to an organic electroluminescent material or an organic electroluminescent device including an organic electroluminescent compound represented by any one of Formulas 3-1-1 to 3-4-1.

Herein, the term “organic electroluminescent material” means a material that may be used in an organic electroluminescent device, and this may comprise at least one compound. The organic electroluminescent material may be comprised in any layer constituting an organic electroluminescent device, as necessary. For example, the organic electroluminescent material may be a hole injection material, a hole transport material, a hole auxiliary material, a light-emitting auxiliary material, an electron-blocking material, a light-emitting material (containing host and dopant materials), an electron buffer material, a hole-blocking material, an electron transport material, or an electron injection material, etc.

Herein, the term “a plurality of host materials” means host materials comprising a combination of at least two compounds, which may be comprised in any light-emitting layer constituting an organic electroluminescent device. It may mean both a material before being comprised in an organic electroluminescent device (e.g., before vapor deposition) and a material after being comprised in an organic electroluminescent device (e.g., after vapor deposition). In one embodiment, a plurality of host materials of the present disclosure may be a combination of at least two host materials, and optionally, it may further include a conventional material included in the organic electroluminescent material. The at least two compounds comprised in a plurality of host materials may be comprised together in one light-emitting layer through methods used in the art, or may each be comprised in separate light-emitting layers. For example, such at least two compounds may be mixture-evaporated or co-evaporated, or may be individually evaporated.

In the present disclosure, the “hole transport band” refers to a region in which holes move between the first electrode and the light-emitting layer, and may include, for example, one or more of a hole injection layer, a hole transport layer, a hole auxiliary layer, a light-emitting auxiliary layer, and an electron-blocking layer. Each of the hole injection layer, the hole transport layer, the hole auxiliary layer, the light-emitting auxiliary layer, and the electron-blocking layer may be a single layer, two or more layers, or a plurality of layers in which three or more layers are stacked. In one embodiment, the hole transport band may include a first hole transport layer and a second hole transport layer, and may further include a third hole transport layer. The second hole transport layer and the third hole transport layer may be one or more layers of a plurality of hole transport layers, and may include one or more of a hole auxiliary layer, a light-emitting auxiliary layer, and an electron-blocking layer. In addition, in another embodiment, the hole transport band may include a first hole transport layer and a second hole transport layer, and the first hole transport layer may be positioned between the first electrode and the light-emitting layer, the second hole transport layer may be positioned between the first hole transport layer and the light-emitting layer, and the second hole transport layer may serve as a hole transport layer, a light-emitting auxiliary layer, a hole auxiliary layer, and/or an electron-blocking layer. In still another embodiment, the hole transport band may include a first hole transport layer, a second hole transport layer, and a third hole transport layer, and the first hole transport layer may be positioned between the first electrode and the light-emitting layer, the second hole transport layer may be positioned between the first hole transport layer and the light-emitting layer, the third hole transport layer may be positioned between the second hole transport layer and the light-emitting layer, and the third hole transport layer may be a layer serving as a hole transport layer, a light-emitting auxiliary layer, a hole auxiliary layer, and/or an electron-blocking layer.

Herein, “(C₁-C₃₀)alkyl(ene)” is meant to be a linear or branched alkyl having 1 to 30 carbon atoms constituting the chain, in which the number of carbon atoms is preferably 1 to 20, more preferably 1 to 10. The above alkyl may include methyl, ethyl, n-propyl, isopropyl, n-butyl, isobutyl, tert-butyl, sec-butyl, etc.

Herein, “(C₃-C₃₀)cycloalkyl(ene)” is meant to be a mono- or polycyclic hydrocarbon having 3 to 30 ring backbone carbon atoms, in which the number of carbon atoms is preferably 3 to 20, more preferably 3 to 7. The above cycloalkyl may include cyclopropyl, cyclobutyl, cyclopentyl, cyclohexyl, cyclopentylmethyl, cyclohexylmethyl, etc.

The “(C₆-C₃₀)aryl(ene)” in the present disclosure is meant to be a monocyclic or fused ring radical derived from an aromatic hydrocarbon having 6 to 30 ring backbone carbon atoms, in which the number of the ring backbone carbon atoms is preferably 6 to 20, more preferably 6 to 15. The above aryl may be partially saturated and may comprise a spiro structure. Examples of the aryl specifically include phenyl, biphenyl, terphenyl, quaterphenyl, naphthyl, binaphthyl, phenylnaphthyl, naphthylphenyl, fluorenyl, phenylfluorenyl, dimethylfluorenyl, diphenylfluorenyl, benzofluorenyl, diphenylbenzofluorenyl, dibenzofluorenyl, phenanthrenyl, benzophenanthrenyl, phenylphenanthrenyl, anthracenyl, benzanthracenyl, indenyl, triphenylenyl, pyrenyl, tetracenyl, perylenyl, chrysenyl, benzochrysenyl, naphthacenyl, fluoranthenyl, benzofluoranthenyl, tolyl, xylyl, mesityl, cumenyl, spiro[fluorene-fluorene]yl, spiro[fluorene-benzofluorene]yl, azulenyl, tetramethyl-dihydrophenanthrenyl, etc. More specifically, the aryl may be o-tolyl, m-tolyl, p-tolyl, 2,3-xylyl, 3,4-xylyl, 2,5-xylyl, mesityl, o-cumenyl, m-cumenyl, p-cumenyl, p-t-butylphenyl, p-(2-phenylpropyl)phenyl, 4′-methylbiphenyl, 4″-t-butyl-p-terphenyl-4-yl, o-biphenyl, m-biphenyl, p-biphenyl, o-terphenyl, m-terphenyl-4-yl, m-terphenyl-3-yl, m-terphenyl-2-yl, p-terphenyl-4-yl, p-terphenyl-3-yl, p-terphenyl-2-yl, m-quaterphenyl, 1-naphthyl, 2-naphthyl, 1-fluorenyl, 2-fluorenyl, 3-fluorenyl, 4-fluorenyl, 9-fluorenyl, 9,9-dimethyl-1-fluorenyl, 9,9-dimethyl-2-fluorenyl, 9,9-dimethyl-3-fluorenyl, 9,9-dimethyl-4-fluorenyl, 9,9-diphenyl-1-fluorenyl, 9,9-diphenyl-2-fluorenyl, 9,9-diphenyl-3-fluorenyl, 9,9-diphenyl-4-fluorenyl, 1-anthryl, 2-anthryl, 9-anthryl, 1-phenanthryl, 2-phenanthryl, 3-phenanthryl, 4-phenanthryl, 9-phenanthryl, 1-chrysenyl, 2-chrysenyl, 3-chrysenyl, 4-chrysenyl, 5-chrysenyl, 6-chrysenyl, benzo[c]phenanthryl, benzo[g]chrysenyl, 1-triphenylenyl, 2-triphenylenyl, 3-triphenylenyl, 4-triphenylenyl, 3-fluoranthenyl, 4-fluoranthenyl, 8-fluoranthenyl, 9-fluoranthenyl, benzofluoranthenyl, 11,11-dimethyl-1-benzo[a]fluorenyl, 11,11-dimethyl-2-benzo[a]fluorenyl, 11,11-dimethyl-3-benzo[a]fluorenyl, 11,11-dimethyl-4-benzo[a]fluorenyl, 11,11-dimethyl-5-benzo[a]fluorenyl, 11,11-dimethyl-6-benzo[a]fluorenyl, 11,11-dimethyl-7-benzo[a]fluorenyl, 11,11-dimethyl-8-benzo[a]fluorenyl, 11,11-dimethyl-9-benzo[a]fluorenyl, 11,11-dimethyl-10-benzo[a]fluorenyl, 11,11-dimethyl-1-benzo[b]fluorenyl, 11,11-dimethyl-2-benzo[b]fluorenyl, 11,11-dimethyl-3-benzo[b]fluorenyl, 11,11-dimethyl-4-benzo[b]fluorenyl, 11,11-dimethyl-5-benzo[b]fluorenyl, 11,11-dimethyl-6-benzo[b]fluorenyl, 11,11-dimethyl-7-benzo[b]fluorenyl, 11,11-dimethyl-8-benzo[b]fluorenyl, 11,11-dimethyl-9-benzo[b]fluorenyl, 11,11-dimethyl-10-benzo[b]fluorenyl, 11,11-dimethyl-1-benzo[c]fluorenyl, 11,11-dimethyl-2-benzo[c]fluorenyl, 11,11-dimethyl-3-benzo[c]fluorenyl, 11,11-dimethyl-4-benzo[c]fluorenyl, 11,11-dimethyl-5-benzo[c]fluorenyl, 11,11-dimethyl-6-benzo[c]fluorenyl, 11,11-dimethyl-7-benzo[c]fluorenyl, 11,11-dimethyl-8-benzo[c]fluorenyl, 11,11-dimethyl-9-benzo[c]fluorenyl, 11,11-dimethyl-10-benzo[c]fluorenyl, 11,11-diphenyl-1-benzo[a]fluorenyl, 11,11-diphenyl-2-benzo[a]fluorenyl, 11,11-diphenyl-3-benzo[a]fluorenyl, 11,11-diphenyl-4-benzo[a]fluorenyl, 11,11-diphenyl-5-benzo[a]fluorenyl, 11,11-diphenyl-6-benzo[a]fluorenyl, 11,11-diphenyl-7-benzo[a]fluorenyl, 11,11-diphenyl-8-benzo[a]fluorenyl, 11,11-diphenyl-9-benzo[a]fluorenyl, 11,11-diphenyl-10-benzo[a]fluorenyl, 11,11-diphenyl-1-benzo[b]fluorenyl, 11,11-diphenyl-2-benzo[b]fluorenyl, 11,11-diphenyl-3-benzo[b]fluorenyl, 11,11-diphenyl-4-benzo[b]fluorenyl, 11,11-diphenyl-5-benzo[b]fluorenyl, 11,11-diphenyl-6-benzo[b]fluorenyl, 11,11-diphenyl-7-benzo[b]fluorenyl, 11,11-diphenyl-8-benzo[b]fluorenyl, 11,11-diphenyl-9-benzo[b]fluorenyl, 11,11-diphenyl-10-benzo[b]fluorenyl, 11,11-diphenyl-1-benzo[c]fluorenyl, 11,11-diphenyl-2-benzo[c]fluorenyl, 11,11-diphenyl-3-benzo[c]fluorenyl, 11,11-diphenyl-4-benzo[c]fluorenyl, 11,11-diphenyl-5-benzo[c]fluorenyl, 11,11-diphenyl-6-benzo[c]fluorenyl, 11,11-diphenyl-7-benzo[c]fluorenyl, 11,11-diphenyl-8-benzo[c]fluorenyl, 11,11-diphenyl-9-benzo[c]fluorenyl, 11,11-diphenyl-10-benzo[c]fluorenyl, 9,9,10,10-tetramethyl-9,10-dihydro-1-phenanthrenyl, 9,9,10,10-tetramethyl-9,10-dihydro-2-phenanthrenyl, 9,9,10,10-tetramethyl-9,10-dihydro-3-phenanthrenyl, 9,9,10,10-tetramethyl-9,10-dihydro-4-phenanthrenyl, etc.

The “(3- to 30-membered)heteroaryl(ene)” in the present disclosure is an aryl having 3 to 30 ring backbone atoms and including at least one heteroatom selected from the group consisting of B, N, O, S, Si, P, Se, and Ge in which the number of the ring backbone atoms is preferably 3 to 30, more preferably 5 to 20. The number of the heteroatoms in the heteroaryl is preferably 1 to 4. The above heteroaryl or heteroarylene may be a monocyclic ring, or a fused ring condensed with at least one benzene ring, and may be partially saturated. Also, the above heteroaryl or heteroarylene herein may be one formed by linking at least one heteroaryl or aryl group to a heteroaryl group via a single bond(s), and may include a spiro structure. Examples of the heteroaryl specifically may include a monocyclic ring-type heteroaryl including furyl, thiophenyl, pyrrolyl, imidazolyl, pyrazolyl, thiazolyl, thiadiazolyl, isothiazolyl, isoxazolyl, oxazolyl, oxadiazolyl, triazinyl, tetrazinyl, triazolyl, tetrazolyl, furazanyl, pyridyl, pyrazinyl, pyrimidinyl, pyridazinyl, etc., and a fused ring-type heteroaryl including benzofuranyl, benzothiophenyl, isobenzofuranyl, dibenzofuranyl, dibenzothiophenyl, dibenzoselenophenyl, benzofuroquinolinyl, benzofuroquinazolinyl, benzofuronaphthiridinyl, benzofuropyrimidinyl, naphthofuropyrimidinyl, benzothienoquinolinyl, benzothienoquinazolinyl, benzothienonaphthiridinyl, benzothienopyrimidinyl, naphthothienopyrimidinyl, pyrimidoindolyl, benzopyrimidoindolyl, benzofuropyrazinyl, naphthofuropyrazinyl, benzothienopyrazinyl, naphthothienopyrazinyl, pyrazinoindolyl, benzopyrazinoindolyl, benzoimidazolyl, benzothiazolyl, benzoisothiazolyl, benzoisoxazolyl, benzoxazolyl, imidazopyridinyl, isoindolyl, indolyl, benzoindolyl, indazolyl, benzothiadiazolyl, quinolyl, isoquinolyl, cinnolinyl, quinazolinyl, quinoxalinyl, carbazolyl, azacarbazolyl, benzocarbazolyl, dibenzocarbazolyl, phenoxazinyl, phenanthridinyl, benzodioxolyl, indolizidinyl, acridinyl, silafluorenyl, germafluorenyl, benzotriazolyl, phenazinyl, imidazopyridinyl, chromenoquinazolinyl, thiochromenoquinazolinyl, dimethylbenzopyrimidinyl, indolocarbazolyl, indenocarbazolyl, etc. More specifically, the heteroaryl may be 1-pyrrolyl, 2-pyrrolyl, 3-pyrrolyl, 2-pyridinyl, 3-pyridinyl, 4-pyridinyl, 2-pyrimidinyl, 4-pyrimidinyl, 5-pyrimidinyl, 6-pyrimidinyl, 1,2,3-triazin-4-yl, 1,2,4-triazin-3-yl, 1,3,5-triazin-2-yl, 1-imidazolyl, 2-imidazolyl, 1-pyrazolyl, 1-indolizidinyl, 2-indolizidinyl, 3-indolizidinyl, 5-indolizidinyl, 6-indolizidinyl, 7-indolizidinyl, 8-indolizidinyl, 2-imidazopyridinyl, 3-imidazopyridinyl, 5-imidazopyridinyl, 6-imidazopyridinyl, 7-imidazopyridinyl, 8-imidazopyridinyl, 1-indolyl, 2-indolyl, 3-indolyl, 4-indolyl, 5-indolyl, 6-indolyl, 7-indolyl, 1-isoindolyl, 2-isoindolyl, 3-isoindolyl, 4-isoindolyl, 5-isoindolyl, 6-isoindolyl, 7-isoindolyl, 2-furyl, 3-furyl, 2-benzofuranyl, 3-benzofuranyl, 4-benzofuranyl, 5-benzofuranyl, 6-benzofuranyl, 7-benzofuranyl, 1-isobenzofuranyl, 3-isobenzofuranyl, 4-isobenzofuranyl, 5-isobenzofuranyl, 6-isobenzofuranyl, 7-isobenzofuranyl, 2-quinolyl, 3-quinolyl, 4-quinolyl, 5-quinolyl, 6-quinolyl, 7-quinolyl, 8-quinolyl, 1-isoquinolyl, 3-isoquinolyl, 4-isoquinolyl, 5-isoquinolyl, 6-isoquinolyl, 7-isoquinolyl, 8-isoquinolyl, 2-quinoxalinyl, 5-quinoxalinyl, 6-quinoxalinyl, 1-carbazolyl, 2-carbazolyl, 3-carbazolyl, 4-carbazolyl, 9-carbazolyl, azacarbazol-1-yl, azacarbazol-2-yl, azacarbazol-3-yl, azacarbazol-4-yl, azacarbazol-5-yl, azacarbazol-6-yl, azacarbazol-7-yl, azacarbazol-8-yl, azacarbazol-9-yl, 1-phenanthridinyl, 2-phenanthridinyl, 3-phenanthridinyl, 4-phenanthridinyl, 6-phenanthridinyl, 7-phenanthridinyl, 8-phenanthridinyl, 9-phenanthridinyl, 10-phenanthridinyl, 1-acridinyl, 2-acridinyl, 3-acridinyl, 4-acridinyl, 9-acridinyl, 2-oxazolyl, 4-oxazolyl, 5-oxazolyl, 2-oxadiazolyl, 5-oxadiazolyl, 3-furazanyl, 2-thienyl, 3-thienyl, 2-methylpyrrol-1-yl, 2-methylpyrrol-3-yl, 2-methylpyrrol-4-yl, 2-methylpyrrol-5-yl, 3-methylpyrrol-1-yl, 3-methylpyrrol-2-yl, 3-methylpyrrol-4-yl, 3-methylpyrrol-5-yl, 2-t-butylpyrrol-4-yl, 3-(2-phenylpropyl)pyrrol-1-yl, 2-methyl-1-indolyl, 4-methyl-1-indolyl, 2-methyl-3-indolyl, 4-methyl-3-indolyl, 2-t-butyl-1-indolyl, 4-t-butyl-1-indolyl, 2-t-butyl-3-indolyl, 4-t-butyl-3-indolyl, 1-dibenzofuranyl, 2-dibenzofuranyl, 3-dibenzofuranyl, 4-dibenzofuranyl, 1-dibenzothiophenyl, 2-dibenzothiophenyl, 3-dibenzothiophenyl, 4-dibenzothiophenyl, 1-naphtho-[1,2-b]-benzofuranyl, 2-naphtho-[1,2-b]-benzofuranyl, 3-naphtho-[1,2-b]-benzofuranyl, 4-naphtho-[1,2-b]-benzofuranyl, 5-naphtho-[1,2-b]-benzofuranyl, 6-naphtho-[1,2-b]-benzofuranyl, 7-naphtho-[1,2-b]-benzofuranyl, 8-naphtho-[1,2-b]-benzofuranyl, 9-naphtho-[1,2-b]-benzofuranyl, 10-naphtho-[1,2-b]-benzofuranyl, 1-naphtho-[2,3-b]-benzofuranyl, 2-naphtho-[2,3-b]-benzofuranyl, 3-naphtho-[2,3-b]-benzofuranyl, 4-naphtho-[2,3-b]-benzofuranyl, 5-naphtho-[2,3-b]-benzofuranyl, 6-naphtho-[2,3-b]-benzofuranyl, 7-naphtho-[2,3-b]-benzofuranyl, 8-naphtho-[2,3-b]-benzofuranyl, 9-naphtho-[2,3-b]-benzofuranyl, 10-naphtho-[2,3-b]-benzofuranyl, 1-naphtho-[2,1-b]-benzofuranyl, 2-naphtho-[2,1-b]-benzofuranyl, 3-naphtho-[2,1-b]-benzofuranyl, 4-naphtho-[2,1-b]-benzofuranyl, 5-naphtho-[2,1-b]-benzofuranyl, 6-naphtho-[2,1-b]-benzofuranyl, 7-naphtho-[2,1-b]-benzofuranyl, 8-naphtho-[2,1-b]-benzofuranyl, 9-naphtho-[2,1-b]-benzofuranyl, 10-naphtho-[2,1-b]-benzofuranyl, 1-naphtho-[1,2-b]-benzothiophenyl, 2-naphtho-[1,2-b]-benzothiophenyl, 3-naphtho-[1,2-b]-benzothiophenyl, 4-naphtho-[1,2-b]-benzothiophenyl, 5-naphtho-[1,2-b]-benzothiophenyl, 6-naphtho-[1,2-b]-benzothiophenyl, 7-naphtho-[1,2-b]-benzothiophenyl, 8-naphtho-[1,2-b]-benzothiophenyl, 9-naphtho-[1,2-b]-benzothiophenyl, 10-naphtho-[1,2-b]-benzothiophenyl, 1-naphtho-[2,3-b]-benzothiophenyl, 2-naphtho-[2,3-b]-benzothiophenyl, 3-naphtho-[2,3-b]-benzothiophenyl, 4-naphtho-[2,3-b]-benzothiophenyl, 5-naphtho-[2,3-b]-benzothiophenyl, 1-naphtho-[2,1-b]-benzothiophenyl, 2-naphtho-[2,1-b]-benzothiophenyl, 3-naphtho-[2,1-b]-benzothiophenyl, 4-naphtho-[2,1-b]-benzothiophenyl, 5-naphtho-[2,1-b]-benzothiophenyl, 6-naphtho-[2,1-b]-benzothiophenyl, 7-naphtho-[2,1-b]-benzothiophenyl, 8-naphtho-[2,1-b]-benzothiophenyl, 9-naphtho-[2,1-b]-benzothiophenyl, 10-naphtho-[2,1-b]-benzothiophenyl, 2-benzofuro[3,2-d]pyrimidinyl, 6-benzofuro[3,2-d]pyrimidinyl, 7-benzofuro[3,2-d]pyrimidinyl, 8-benzofuro[3,2-d]pyrimidinyl, 9-benzofuro[3,2-d]pyrimidinyl, 2-benzothio[3,2-d]pyrimidinyl, 6-benzothio[3,2-d]pyrimidinyl, 7-benzothio[3,2-d]pyrimidinyl, 8-benzothio[3,2-d]pyrimidinyl, 9-benzothio[3,2-d]pyrimidinyl, 2-benzofuro[3,2-d]pyrazinyl, 6-benzofuro[3,2-d]pyrazinyl, 7-benzofuro[3,2-d]pyrazinyl, 8-benzofuro[3,2-d]pyrazinyl, 9-benzofuro[3,2-d]pyrazinyl, 2-benzothio[3,2-d]pyrazinyl, 6-benzothio[3,2-d]pyrazinyl, 7-benzothio[3,2-d]pyrazinyl, 8-benzothio[3,2-d]pyrazinyl, 9-benzothio[3,2-d]pyrazinyl, 1-silafluorenyl, 2-silafluorenyl, 3-silafluorenyl, 4-silafluorenyl, 1-germafluorenyl, 2-germafluorenyl, 3-germafluorenyl, 4-germafluorenyl, 1-dibenzoselenophenyl, 2-dibenzoselenophenyl, 3-dibenzoselenophenyl, 4-dibenzoselenophenyl, etc. Additionally, “heteroaryl(ene)” can be classified into a heteroaryl(ene) with electronic properties and a heteroaryl(ene) with hole properties. A heteroaryl(ene) with electronic properties is a substituent with relatively abundant electrons in the parent nucleus, and for example, it may be a substituted or unsubstituted pyridinyl, a substituted or unsubstituted pyrimidinyl, a substituted or unsubstituted triazinyl, a substituted or unsubstituted quinazolinyl, a substituted or unsubstituted quinoxalinyl, a substituted or unsubstituted quinolyl, etc. A heteroaryl(ene), which has hole properties, is a substituent with a relative lack of electrons in the parent nucleus, and for example, it may be a substituted or unsubstituted carbazolyl, a substituted or unsubstituted dibenzofuranyl, or a substituted or unsubstituted dibenzothiophenyl.

Herein, “a fused ring of (C₃-C₃₀) aliphatic ring and (C₆-C₃₀) aromatic ring” means a ring formed by fusing at least one aliphatic ring having 3 to 30 ring backbone carbon atoms in which the number of carbon atoms is preferably 3 to 25, more preferably 3 to 18, and at least one aromatic ring having 6 to 30 ring backbone carbon atoms in which the number of carbon atoms is preferably 6 to 25, more preferably 6 to 18. For example, the fused ring may be a fused ring of at least one benzene and at least one cyclohexane, or a fused ring of at least one naphthalene and at least one cyclopentane, etc. Herein, the carbon atoms in the fused ring of (C₃-C₃₀) aliphatic ring and (C₆-C₃₀) aromatic ring may be replaced with at least one heteroatom selected from B, N, O, S, Si, and P, preferably at least one heteroatom selected from N, O, and S. The “halogen” in the present disclosure includes F, Cl, Br, and I.

In addition, “ortho-” (“o-”), “meta-” (“m-”), and “para-” (“p-”) are meant to signify the substitution position of all substituents. An ortho-configuration describes a compound with substituents which are adjacent to each other, e.g., at the 1 and 2 positions on benzene. A meta-configuration indicates the next substitution position of the immediately adjacent substitution position, e.g., a compound with substituents at the 1 and 3 positions on benzene. A para-configuration indicates the next substitution position from the meta-position, e.g., a compound with substituents at the 1 and 4 positions on benzene.

Herein, “a ring formed in linking to an adjacent substituent” means a substituted or unsubstituted (3- to 50-membered) mono- or polycyclic, alicyclic, aromatic ring, or a combination thereof, formed by linking or fusing two or more adjacent substituents, and preferably this may be a substituted or unsubstituted (5- to 40-membered) mono- or polycyclic, alicyclic, aromatic ring, or a combination thereof. Further, the formed ring may include at least one heteroatom selected from the group consisting of B, N, O, S, Si, and P, preferably N, O, and S. According to one embodiment of the present disclosure, the number of ring backbone atoms is 5 to 35; according to another embodiment of the present disclosure, the number of ring backbone atoms is 5 to 30. In one embodiment, the fused ring may be, for example, a substituted or unsubstituted fluorene ring, a substituted or unsubstituted dibenzothiophene ring, a substituted or unsubstituted dibenzofuran ring, a substituted or unsubstituted naphthalene ring, a substituted or unsubstituted phenanthrene ring, a substituted or unsubstituted benzofluorene ring, a substituted or unsubstituted benzothiophene ring, a substituted or unsubstituted benzofuran ring, a substituted or unsubstituted indole ring, a substituted or unsubstituted indene ring, a substituted or unsubstituted benzene ring, or a substituted or unsubstituted carbazole ring, etc.

In addition, in the “substituted or unsubstituted” description herein, the term “substituted” refers to that a hydrogen atom in a functional group is replaced with another atom or another functional group (i.e., a substituent). Unless otherwise specified, the substituent may replace hydrogen at a position where the substituent can be substituted without limitation, and when two or more hydrogen atoms are each replaced with a substituent in a functional group, the substituents may be the same as or different from each other. The maximum number of substituents that can be substituted for a certain functional group may be the total number of valences that can be substituted for each atom forming the functional group. Preferably, the substituted alkyl, the substituted aryl(ene), the substituted heteroaryl(ene), the substituted cycloalkyl, the substituted alkoxy, the substituted trialkylsilyl, the substituted dialkylarylsilyl, the substituted alkyldiarylsilyl, and the substituted triarylsilyl in the formulas of the present disclosure each independently may be substituted with least one selected from the group consisting of deuterium; halogen; cyano; carboxyl; nitro; hydroxy; phosphine oxide; (C₁-C₃₀)alkyl, halo(C₁-C₃₀)alkyl, (C₂-C₃₀)alkenyl, (C₂-C₃₀)alkynyl, (C₁-C₃₀)alkoxy, (C₁-C₃₀)alkylthio, (C₃-C₃₀)cycloalkyl, (C₃-C₃₀)cycloalkenyl, (3- to 7-membered)heterocycloalkyl, (C₆-C₃₀)aryloxy, (C₆-C₃₀)arylthio, (5- to 30-membered)heteroaryl unsubstituted or substituted with (C₆-C₃₀)aryl, (C₆-C₃₀)aryl unsubstituted or substituted with (5- to 30-membered)heteroaryl, tri(C₁-C₃₀)alkylsilyl, tri(C₆-C₃₀)arylsilyl, di(C₁-C₃₀)alkyl(C₆-C₃₀)arylsilyl, (C₁-C₃₀)alkyldi(C₆-C₃₀)arylsilyl, amino, mono- or di(C₁-C₃₀)alkylamino, mono- or di(C₆-C₃₀)arylamino unsubstituted or substituted with (C₁-C₃₀)alkyl, (C₁-C₃₀)alkyl(C₆-C₃₀)arylamino, (C₁-C₃₀)alkylcarbonyl, (C₁-C₃₀)alkoxycarbonyl, (C₆-C₃₀)arylcarbonyl, (C₆-C₃₀)arylphosphinyl, di(C₆-C₃₀)arylboronyl, di(C₁-C₃₀)alkylboronyl, (C₁-C₃₀)alkyl(C₆-C₃₀)arylboronyl, (C₆-C₃₀)ar(C₁-C₃₀)alkyl, and (C₁-C₃₀)alkyl(C₆-C₃₀)aryl. For example, the substituent may be deuterium, a substituted or unsubstituted phenyl, a substituted or unsubstituted biphenyl, a substituted or unsubstituted o-terphenyl, a substituted or unsubstituted m-terphenyl, a substituted or unsubstituted p-terphenyl, a substituted or unsubstituted naphthyl, a substituted or unsubstituted phenanthrenyl, a substituted or unsubstituted anthracenyl, a substituted or unsubstituted benzonaphthalenyl, a substituted or unsubstituted benzophenanthrenyl, a substituted or unsubstituted chrysenyl, a substituted or unsubstituted fluorenyl, a substituted or unsubstituted benzofluorenyl, a substituted or unsubstituted spirobifluorenyl, a substituted or unsubstituted fluoranthenyl, a substituted or unsubstituted dibenzonaphthocycloheptanyl, a substituted or unsubstituted pyridinyl, a substituted or unsubstituted benzofuranyl, a substituted or unsubstituted dibenzofuranyl, a substituted or unsubstituted benzonaphthofuranyl, a substituted or unsubstituted dibenzothiophenyl, a substituted or unsubstituted benzonaphthothiophenyl, a substituted or unsubstituted benzofuropyridinyl, a substituted or unsubstituted carbazolyl, a substituted or unsubstituted dibenzoselenophenyl, a substituted or unsubstituted benzonaphthoselenophenyl, a substituted or unsubstituted benzoimidazolyl, a substituted or unsubstituted phenoxazinyl, a substituted or unsubstituted benzoxazolyl, a substituted or unsubstituted benzothiazolyl, a substituted or unsubstituted triphenylsilyl, or a substituted or unsubstituted amino, etc., and the substituted amino may be substituted with one or more selected from the group consisting of phenyl, biphenyl, naphthyl, pyridinyl, dibenzofuranyl, and dibenzothiophenyl.

When a substituent is not shown in the chemical formula or the compound structure, it may signify that all positions that may be present as substituents are hydrogen or deuterium. That is, in the case of deuterium, an isotope of hydrogen, some of the hydrogen atoms may be deuterium, which is an isotope; and in this case, the content of deuterium may be 0% to 100%. In the case where the substituent is not shown in the chemical formula or the compound structure, when deuterium is not explicitly excluded, hydrogen and deuterium may be mixed and used in the compound, such as when the content of deuterium is 0%, the content of hydrogen is 100%, and all substituents are hydrogen. The deuterium is an element having a deuteron composed of one proton and one neutron as an atomic nucleus, which is one of the isotopes of hydrogen, and may be represented by hydrogen-2, and the element symbol may be D or ²H. The isotope having the same atomic number (Z) but having a different mass number (A) may also be interpreted as an element having the same number of protons but having a different number of neutrons.

Herein, “combinations thereof” signifies that one or more components of the corresponding list are combined to form a known or chemically stable arrangement that a person skilled in the art could conceive from the corresponding list. For example, alkyl and deuterium may be combined to form partially or entirely deuterated alkyl groups; halogen and alkyl may be combined to form halogenated alkyl substituents; and halogen, alkyl, and aryl may be combined to form halogenated arylalkyl. For example, preferred combinations of substituents may include up to 50 atoms excluding hydrogen and deuterium, or include up to 40 atoms excluding hydrogen and deuterium, or include up to 30 atoms excluding hydrogen and deuterium, or in many cases, preferred combinations of substituents may include up to 20 atoms excluding hydrogen and deuterium.

In the formulas of the present disclosure, when multiple substituents are indicated by the same symbol, each of these substituents represented by the same symbol may be identical to or different from one another.

Hereinafter, the host material according to one embodiment will be described in detail.

According to one embodiment, a plurality of host materials include a first host material comprising a compound represented by Formula 1 and a second host material comprising a compound represented by Formula 2, and may be included in at least one light-emitting layer of an organic electroluminescent device according to one embodiment.

The first host material, which is a host material according to one embodiment, may include a compound represented by the following Formula 1.

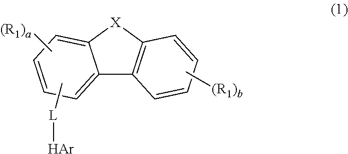

In Formula 1,
X represents O, S, or Se;
HAr represents a substituted or unsubstituted (3- to 30-membered)heteroaryl containing one or more nitrogen atoms;
L represents a single bond, a substituted or unsubstituted (C₆-C₃₀)arylene, or a substituted or unsubstituted (3- to 30-membered)heteroarylene;
R₁ and R₂ each independently represent hydrogen, deuterium, a halogen, a cyano, a substituted or unsubstituted (C₁-C₃₀)alkyl, a substituted or unsubstituted (C₆-C₃₀)aryl, a substituted or unsubstituted (3- to 30-membered)heteroaryl, a substituted or unsubstituted (C₃-C₃₀)cycloalkyl, a substituted or unsubstituted (C₁-C₃₀)alkoxy, a substituted or unsubstituted tri(C₁-C₃₀)alkylsilyl, a substituted or unsubstituted di(C₁-C₃₀)alkyl(C₆-C₃₀)arylsilyl, a substituted or unsubstituted (C₁-C₃₀)alkyldi(C₆-C₃₀)arylsilyl, or a substituted or unsubstituted tri(C₆-C₃₀)arylsilyl; or may be linked to the adjacent substituents to form a ring(s);
provided that at least one of R₁ and R₂ is a substituted or unsubstituted (C₆-C₃₀)aryl, or a substituted or unsubstituted (3- to 30-membered)heteroaryl; and
a represents an integer of 1 to 3, b represents an integer of 1 to 4, and when a and b are an integer of 2 or more, each of R₁ and each of R₂ may be the same as or different from each other.

In one embodiment, X may be O, S, or Se.

In one embodiment, HAr may be a substituted or unsubstituted (3- to 30-membered)heteroaryl containing one or more nitrogen atoms, preferably HAr may be a substituted or unsubstituted (3- to 25-membered)heteroaryl containing one or more nitrogen atoms. For example, HAr may be a substituted or unsubstituted triazinyl, and said substituted triazinyl may be substituted with one or more selected from the group consisting of (C₆-C₃₀)aryl and (3- to 30-membered)heteroaryl, which may be further substituted with deuterium. For example, the substituted triazinyl may be substituted with one or more selected from the group consisting of a substituted or unsubstituted phenyl, a substituted or unsubstituted biphenyl, a substituted or unsubstituted o-terphenyl, a substituted or unsubstituted m-terphenyl, a substituted or unsubstituted p-terphenyl, a substituted or unsubstituted naphthyl, a substituted or unsubstituted phenanthrenyl, a substituted or unsubstituted chrysenyl, a substituted or unsubstituted benzonaphthofuranyl, a substituted or unsubstituted benzophenanthrofuranyl, a substituted or unsubstituted dibenzofuranyl, a substituted or unsubstituted dibenzothiophenyl, a substituted or unsubstituted carbazolyl, a substituted or unsubstituted benzonaphthoselenophenyl, and a substituted or unsubstituted triphenylsilyl, which may be further substituted with one or more selected from the group consisting of deuterium, phenyl, naphthyl, phenylnaphthyl and naphthylphenyl.

In one embodiment, L may be a single bond or a substituted or unsubstituted (C₆-C₃₀)arylene, preferably L may be a single bond or a substituted or unsubstituted (C₆-C₂₅)arylene. For example, L may be a single bond, a substituted or unsubstituted phenylene, or a substituted or unsubstituted naphthylene.

In one embodiment, R₁ and R₂ each independently may be hydrogen, deuterium, a substituted or unsubstituted (C₆-C₃₀)aryl, a substituted or unsubstituted (3- to 30-membered)heteroaryl, or a substituted or unsubstituted (C₃-C₃₀)cycloalkyl, provided that at least one of R₁ and R₂ may be a substituted or unsubstituted (C₆-C₃₀)aryl, or a substituted or unsubstituted (3- to 30-membered)heteroaryl. Preferably, R₁ and R₂ each independently may be hydrogen, deuterium, a substituted or unsubstituted (C₁-C₂₅)alkyl, a substituted or unsubstituted (C₆-C₂₅)aryl, a substituted or unsubstituted (3- to 25-membered)heteroaryl, or a substituted or unsubstituted (C₃-C₂₅)cycloalkyl, provided that at least one of R₁ and R₂ may be a substituted or unsubstituted (C₆-C₂₅)aryl, or a substituted or unsubstituted (3- to 25-membered)heteroaryl. For example, R₁ and R₂ each independently may be hydrogen, deuterium, a substituted or unsubstituted phenyl, a substituted or unsubstituted biphenyl, a substituted or unsubstituted o-terphenyl, a substituted or unsubstituted m-terphenyl, a substituted or unsubstituted p-terphenyl, a substituted or unsubstituted naphthyl, a substituted or unsubstituted phenanthrenyl, a substituted or unsubstituted benzophenanthrenyl, a substituted or unsubstituted chrysenyl, a substituted or unsubstituted fluoranthenyl, a substituted or unsubstituted pyridinyl, a substituted or unsubstituted dibenzofuranyl, a substituted or unsubstituted dibenzothiophenyl, a substituted or unsubstituted carbazolyl, a substituted or unsubstituted benzoxazolyl, or a substituted or unsubstituted benzothiazolyl, which may be further substituted with one or more selected from the group consisting of deuterium, phenyl and naphthyl.

In one embodiment, R₁ represents a substituted or unsubstituted (C₆-C₃₀)aryl.

In one embodiment, a represents an integer of 1 to 3, b represents an integer of 1 to 4, and when a and b are an integer of 2 or more, each of R₁ and each of R₂ may be the same as or different from each other.

The compound represented by Formula 1 can be represented by any one of the following Formulas 1-1 to 1-4.

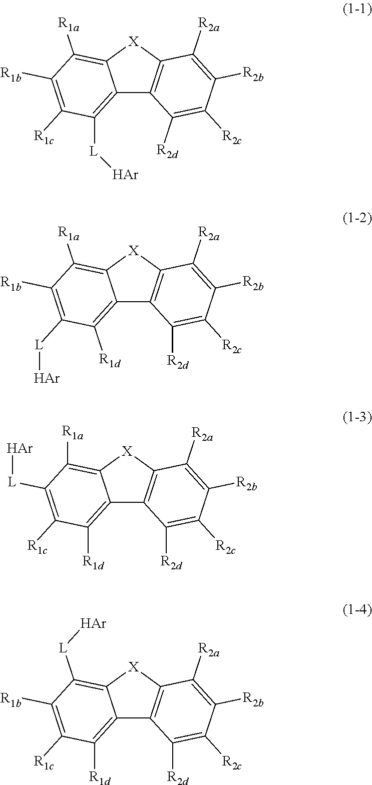

In Formulas 1-1 to 1-4,
R₁ₐ to R_{1d} and R₂ₐ to R_{2d} each independently represent hydrogen, deuterium, a halogen, a cyano, a substituted or unsubstituted (C₁-C₃₀)alkyl, a substituted or unsubstituted (C₆-C₃₀)aryl, a substituted or unsubstituted (3- to 30-membered)heteroaryl, a substituted or unsubstituted (C₃-C₃₀)cycloalkyl, a substituted or unsubstituted (C₁-C₃₀)alkoxy, a substituted or unsubstituted tri(C₁-C₃₀)alkylsilyl, a substituted or unsubstituted di(C₁-C₃₀)alkyl(C₆-C₃₀)arylsilyl, a substituted or unsubstituted (C₁-C₃₀)alkyldi(C₆-C₃₀)arylsilyl, or a substituted or unsubstituted tri(C₆-C₃₀)arylsilyl; or may be linked to the adjacent substituents to form a ring(s);
provided that in each of Formulas 1-1 to 1-4, at least one of R₁ₐ to R_{1d} and R₂ₐ to R_{2d} is a substituted or unsubstituted (C₆-C₃₀)aryl or a substituted or unsubstituted (3- to 30-membered)heteroaryl; and
X, L, and HAr are as defined in Formula 1.

According to one embodiment, the first host material may be more specifically illustrated by the following compounds, but is not limited thereto:

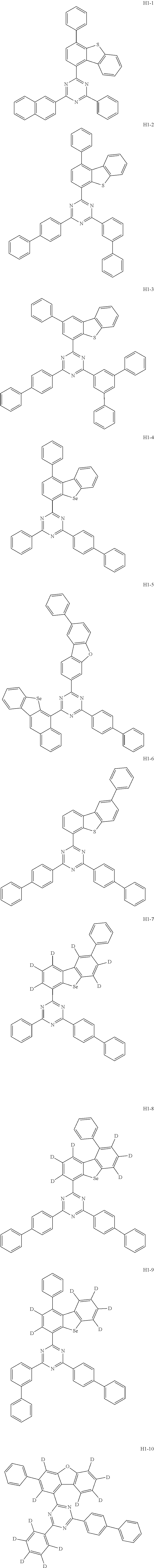

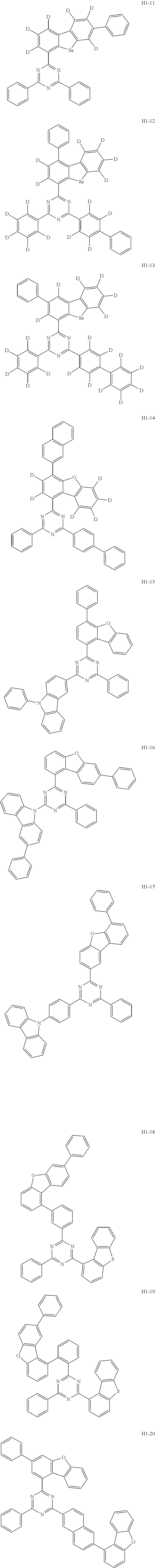

[chemical structure]

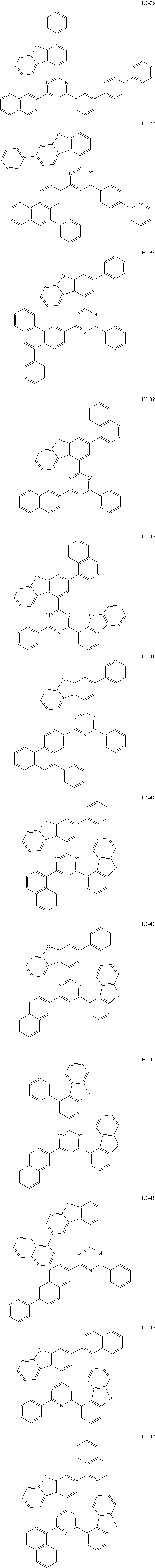

[chemical structure]

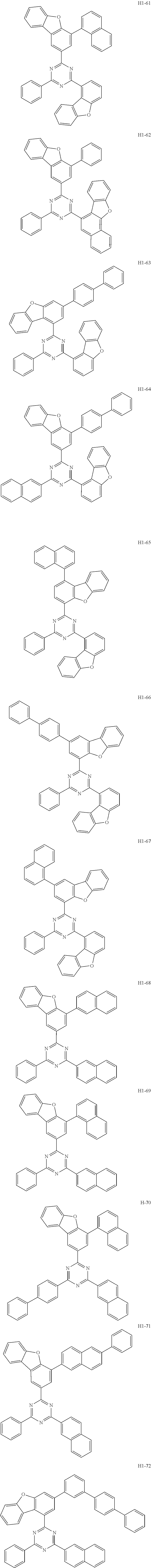

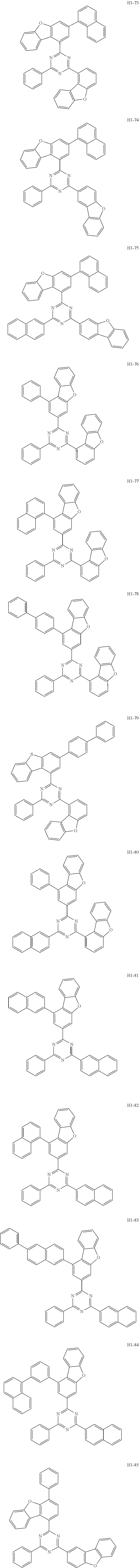

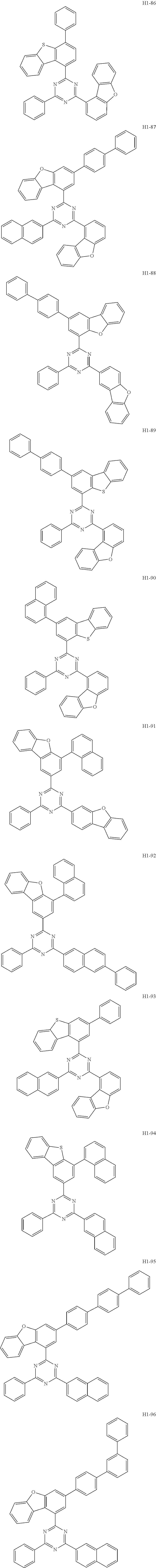

[chemical structure]

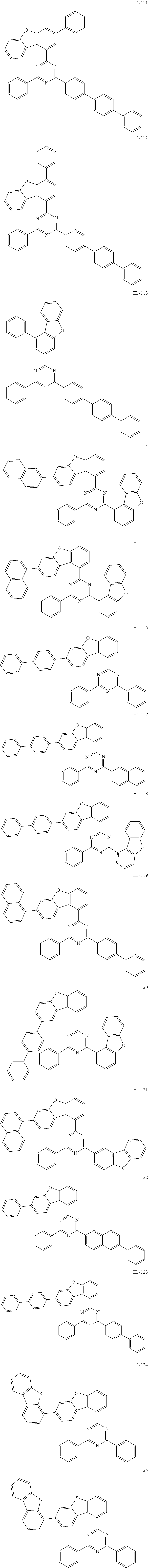

[chemical structure]

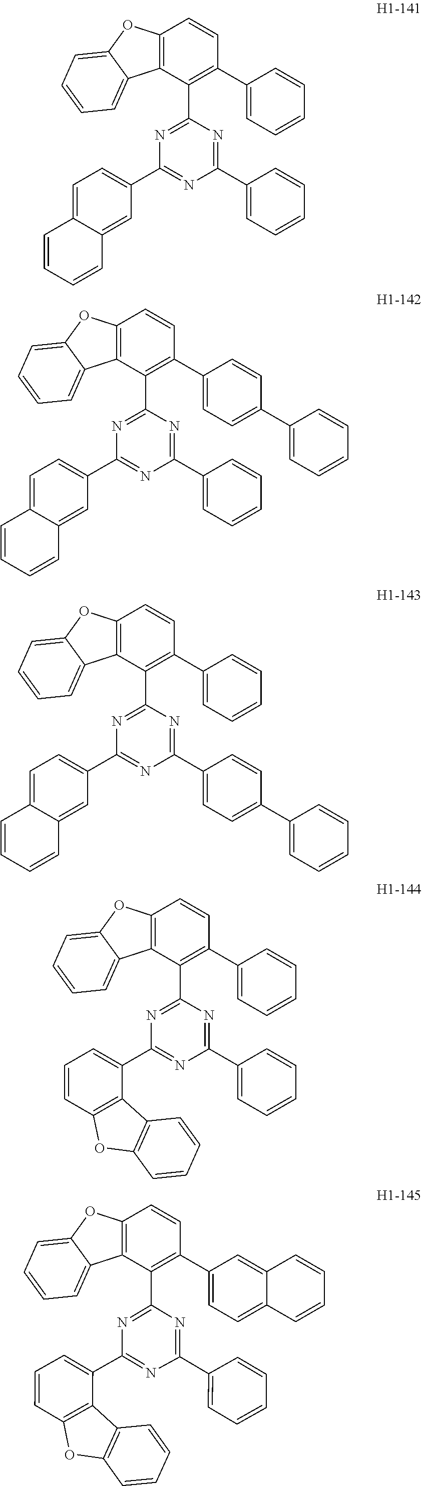

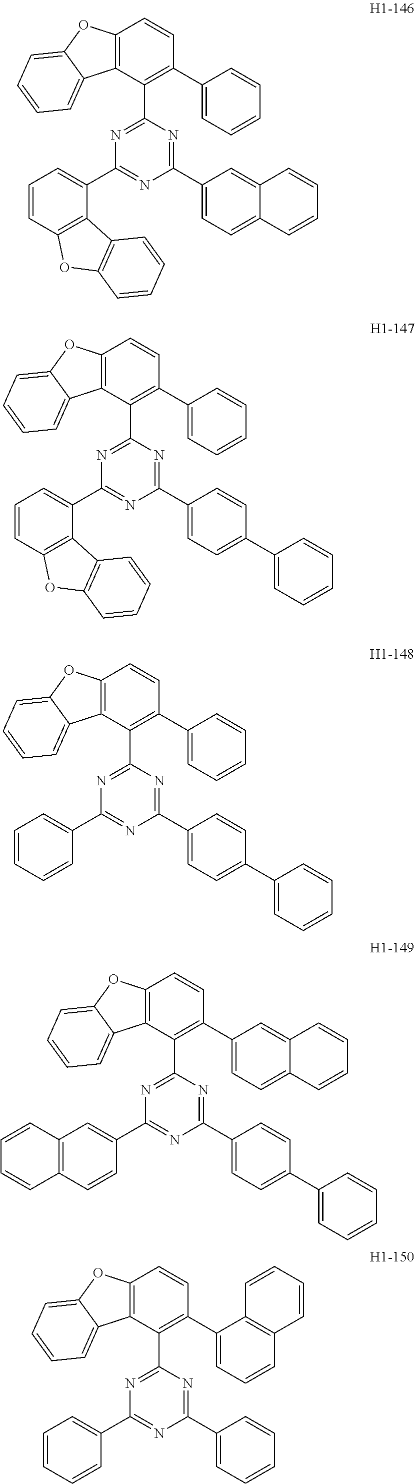

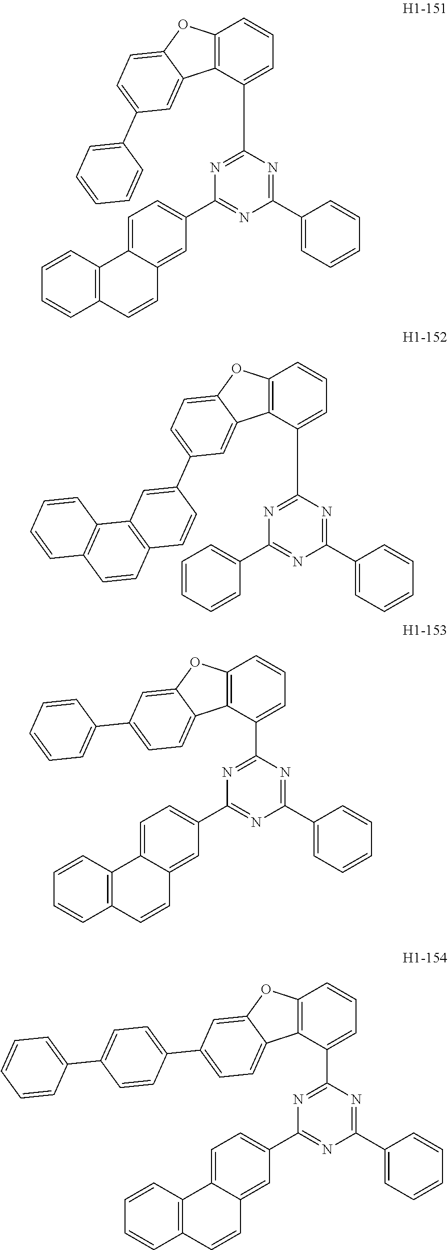 [SMILES: c1ccc(-c2ccc3oc4cccc(-c5nc(-c6ccccc6)nc(-c6ccc7c(ccc8ccccc87)c6)n5)c4c3c2)cc1 ; c1ccc(-c2nc(-c3ccccc3)nc(-c3cccc4oc5ccc(-c6ccc7ccc8ccccc8c7c6)cc5c34)n2)cc1 ; c1ccc(-c2ccc3c(c2)oc2cccc(-c4nc(-c5ccccc5)nc(-c5ccc6c(ccc7ccccc76)c5)n4)c23)cc1 ; c1ccc(-c2ccc(-c3ccc4c(c3)oc3cccc(-c5nc(-c6ccccc6)nc(-c6ccc7c(ccc8ccccc87)c6)n5)c34)cc2)cc1]

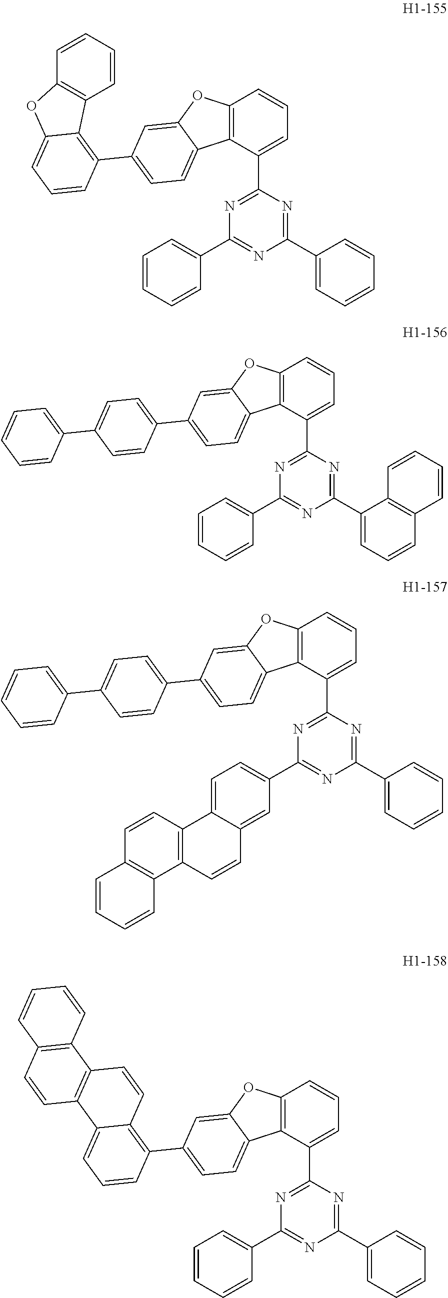

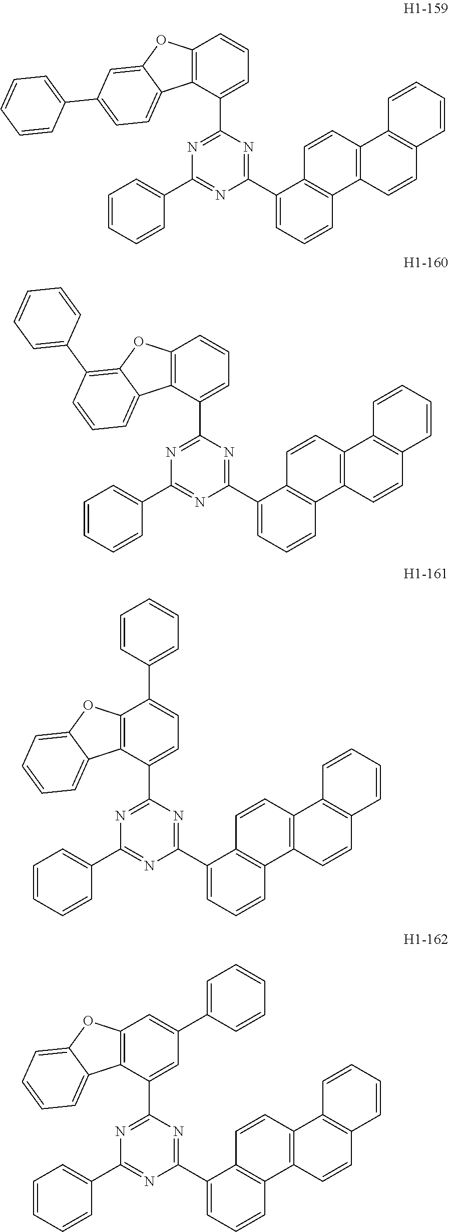 [SMILES: c1ccc(-c2ccc3c(c2)oc2cccc(-c4nc(-c5ccccc5)nc(-c5cccc6c5ccc5c7ccccc7ccc65)n4)c23)cc1 ; c1ccc(-c2nc(-c3cccc4c3ccc3c5ccccc5ccc43)nc(-c3cccc4oc5c(-c6ccccc6)cccc5c34)n2)cc1 ; c1ccc(-c2nc(-c3cccc4c3ccc3c5ccccc5ccc43)nc(-c3ccc(-c4ccccc4)c4oc5ccccc5c34)n2)cc1 ; c1ccc(-c2cc(-c3nc(-c4ccccc4)nc(-c4cccc5c4ccc4c6ccccc6ccc54)n3)c3c(c2)oc2ccccc23)cc1]

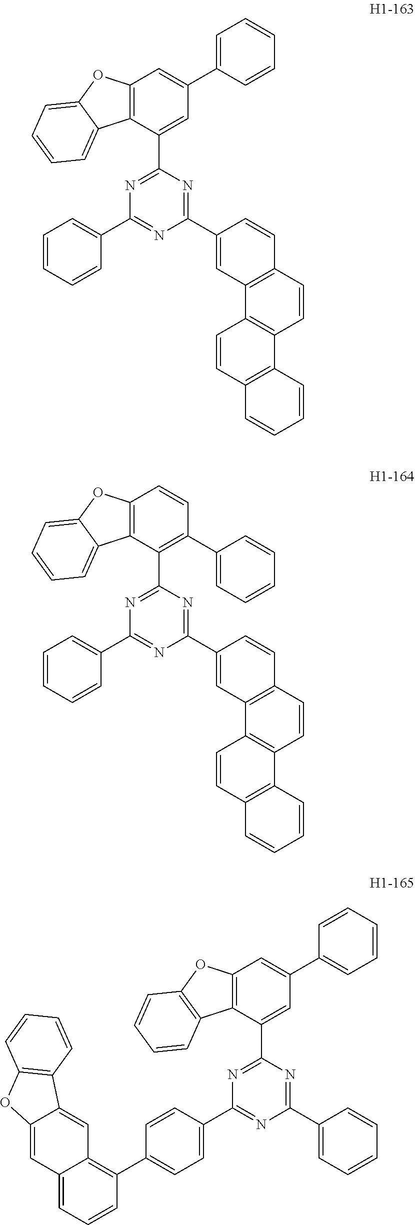

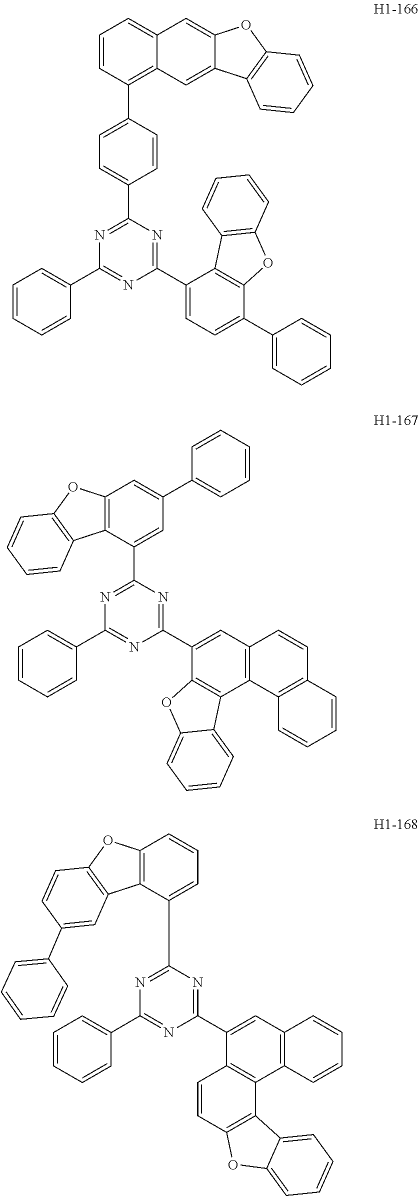 [SMILES: c1ccc(-c2nc(-c3ccc(-c4cccc5cc6oc7ccccc7c6cc45)cc3)nc(-c3ccc(-c4ccccc4)c4oc5ccccc5c34)n2)cc1 ; c1ccc(-c2cc(-c3nc(-c4ccccc4)nc(-c4cc5ccc6ccccc6c5c5c4oc4ccccc45)n3)c3c(c2)oc2ccccc23)cc1 ; c1ccc(-c2ccc3oc4cccc(-c5nc(-c6ccccc6)nc(-c6cc7ccccc7c7c6ccc6oc8ccccc8c67)n5)c4c3c2)cc1]

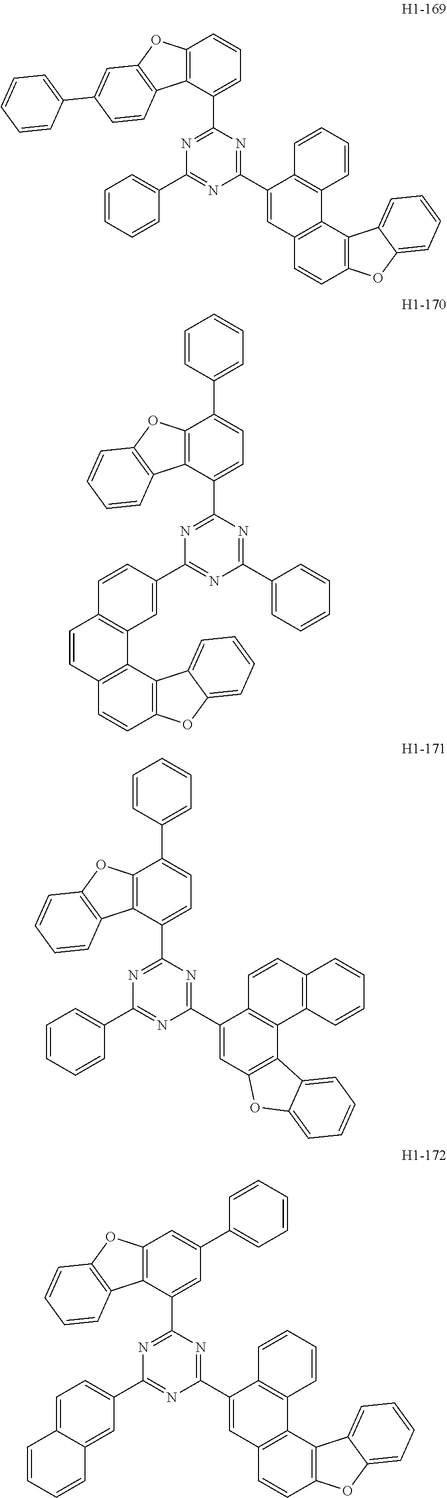

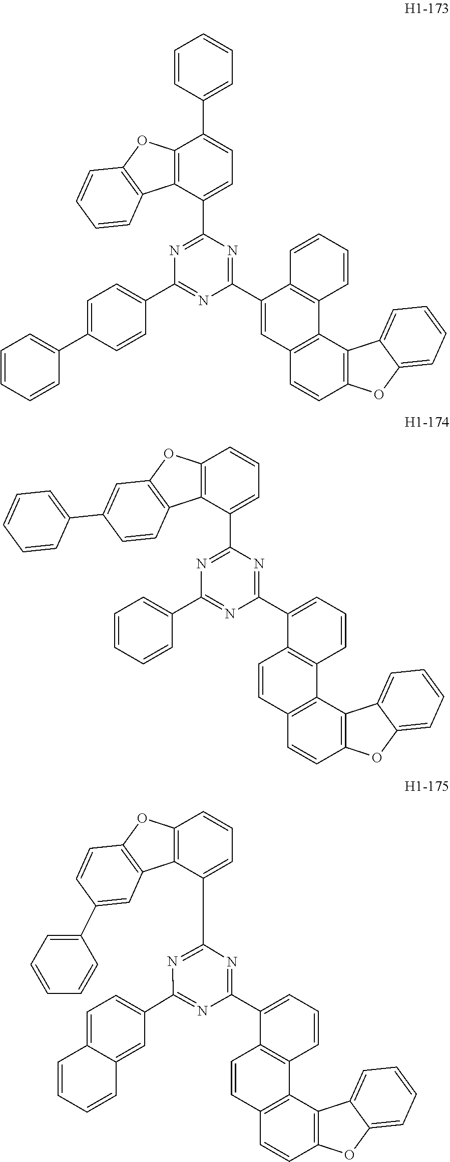

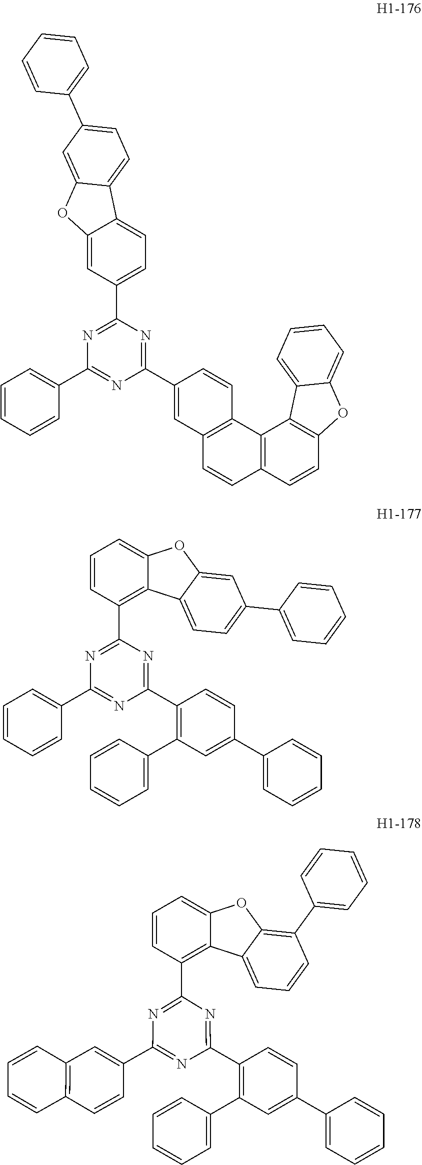

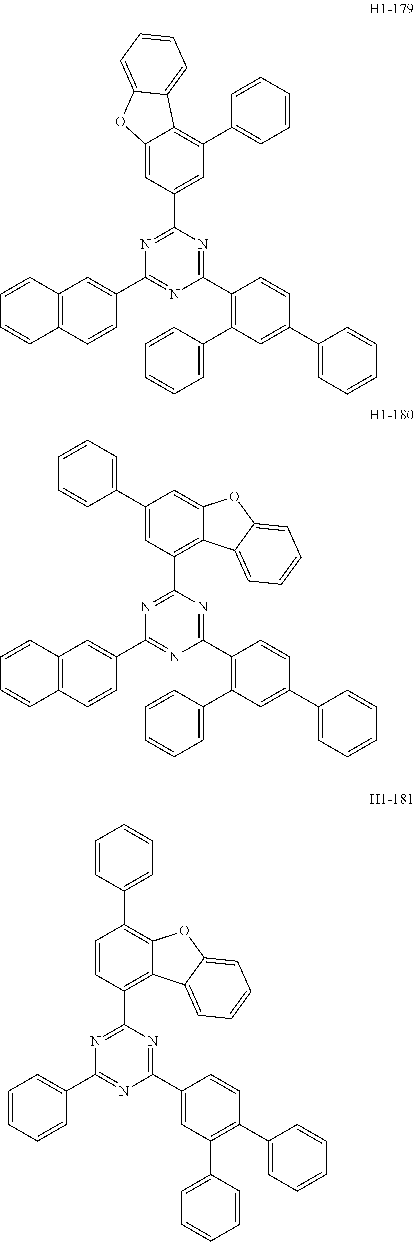

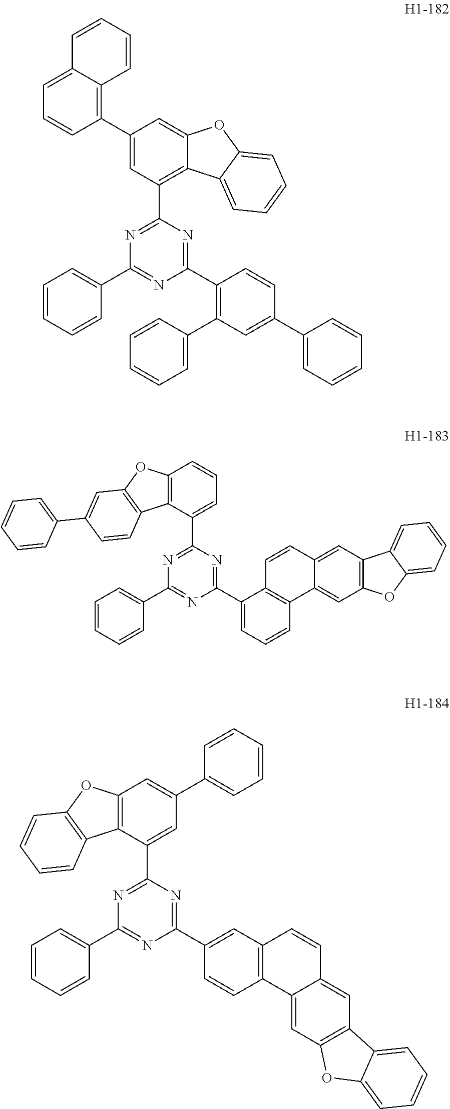 [SMILES: c1ccc(-c2ccc(-c3nc(-c4ccccc4)nc(-c4cc(-c5cccc6ccccc56)cc5oc6ccccc6c45)n3)c(-c3ccccc3)c2)cc1 ; c1ccc(-c2ccc3c(c2)oc2cccc(-c4nc(-c5ccccc5)nc(-c5cccc6c5ccc5cc7c(cc56)oc5ccccc57)n4)c23)cc1 ; c1ccc(-c2cc(-c3nc(-c4ccccc4)nc(-c4ccc5c(ccc6cc7c(cc65)oc5ccccc57)c4)n3)c3c(c2)oc2ccccc23)cc1]

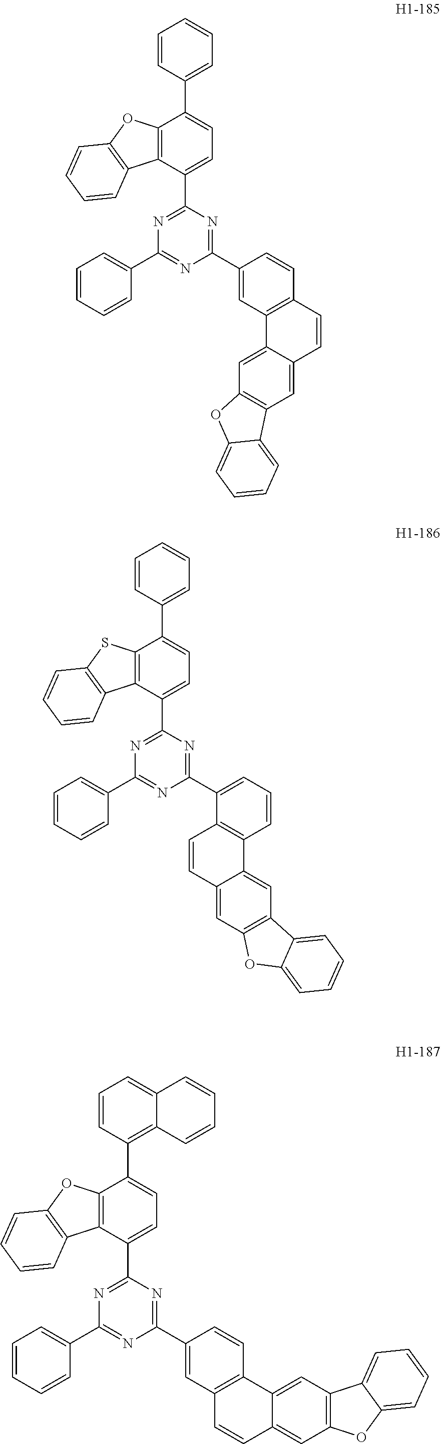 [SMILES: c1ccc(-c2nc(-c3ccc4ccc5cc6c(cc5c4c3)oc3ccccc36)nc(-c3ccc(-c4ccccc4)c4oc5ccccc5c34)n2)cc1 ; c1ccc(-c2nc(-c3cccc4c3ccc3cc5oc6ccccc6c5cc34)nc(-c3ccc(-c4ccccc4)c4sc5ccccc5c34)n2)cc1 ; c1ccc(-c2nc(-c3ccc4c(ccc5cc6oc7ccccc7c6cc54)c3)nc(-c3ccc(-c4cccc5ccccc45)c4oc5ccccc5c34)n2)cc1]

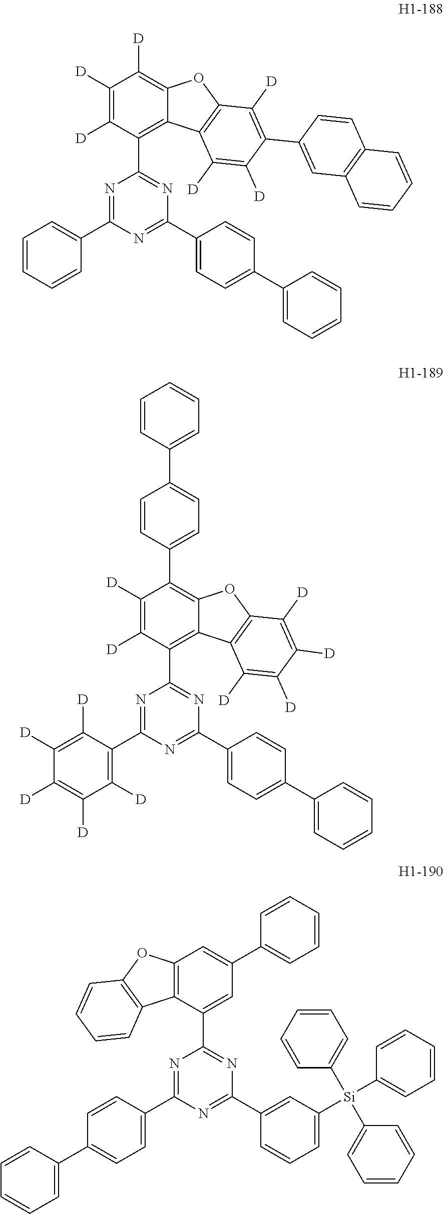 [SMILES: [2H]c1c([2H])c(-c2nc(-c3ccccc3)nc(-c3ccc(-c4ccccc4)cc3)n2)c2c(oc3c([2H])c(-c4ccc5ccccc5c4)c([2H])c([2H])c32)c1[2H] ; [2H]c1c([2H])c([2H])c(-c2nc(-c3ccc(-c4ccccc4)cc3)nc(-c3c([2H])c([2H])c(-c4ccc(-c5ccccc5)cc4)c4oc5c([2H])c([2H])c([2H])c([2H])c5c34)n2)c([2H])c1[2H] ; c1ccc(-c2ccc(-c3nc(-c4cccc([Si](c5ccccc5)(c5ccccc5)c5ccccc5)c4)nc(-c4cc(-c5ccccc5)cc5oc6ccccc6c45)n3)cc2)cc1]

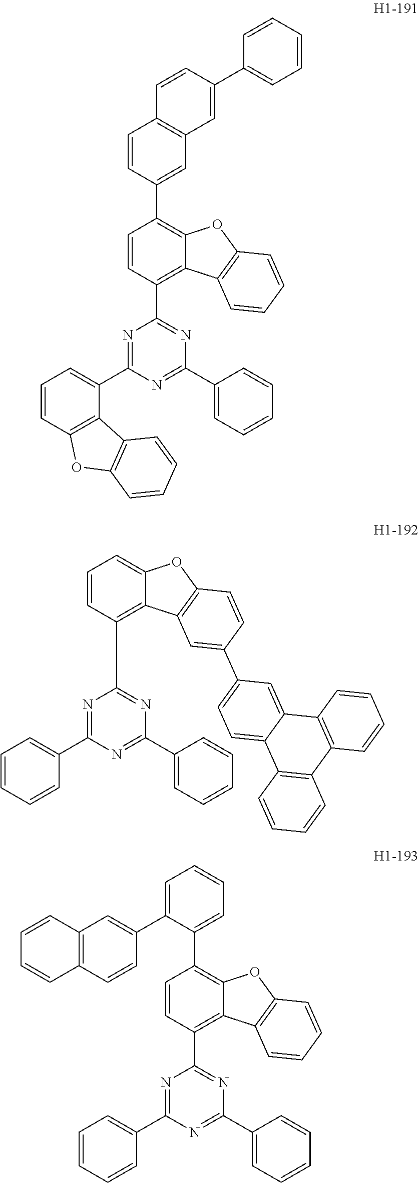 [SMILES: c1ccc(-c2ccc3ccc(-c4ccc(-c5nc(-c6ccccc6)nc(-c6cccc7oc8ccccc8c67)n5)c5c4oc4ccccc45)cc3c2)cc1 ; c1ccc(-c2nc(-c3ccccc3)nc(-c3cccc4oc5ccc(-c6ccc7c8ccccc8c8ccccc8c7c6)cc5c34)n2)cc1 ; c1ccc(-c2nc(-c3ccccc3)nc(-c3ccc(-c4ccccc4-c4ccc5ccccc5c4)c4oc5ccccc5c34)n2)cc1]

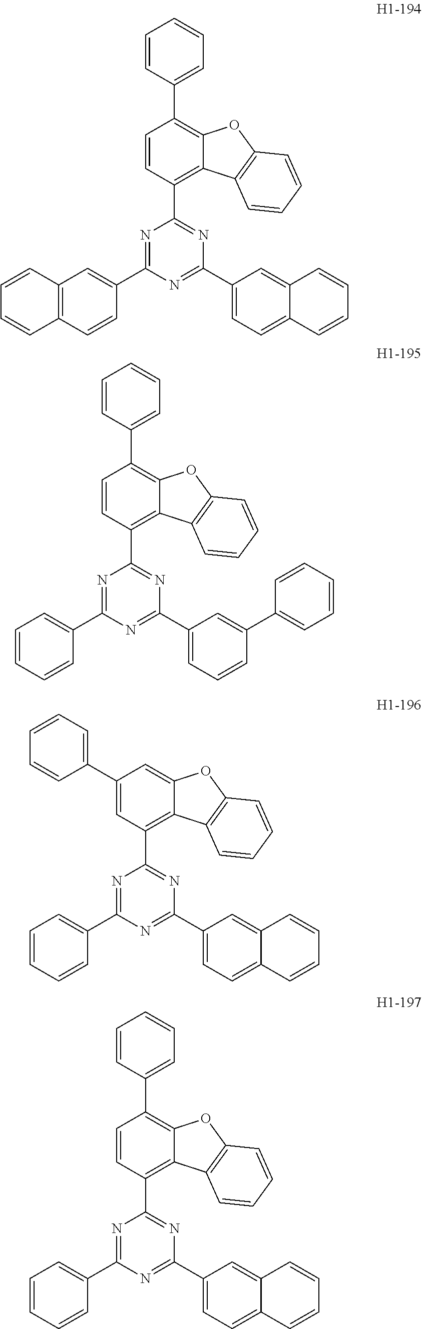

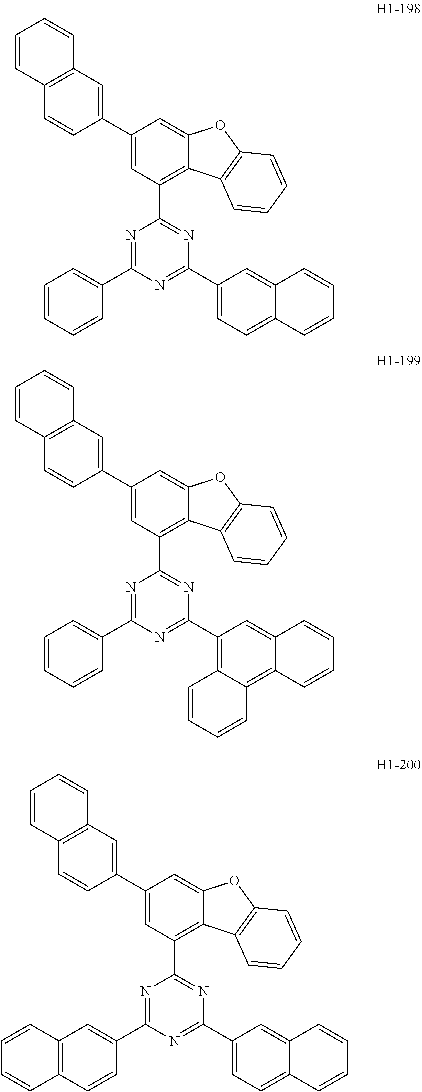

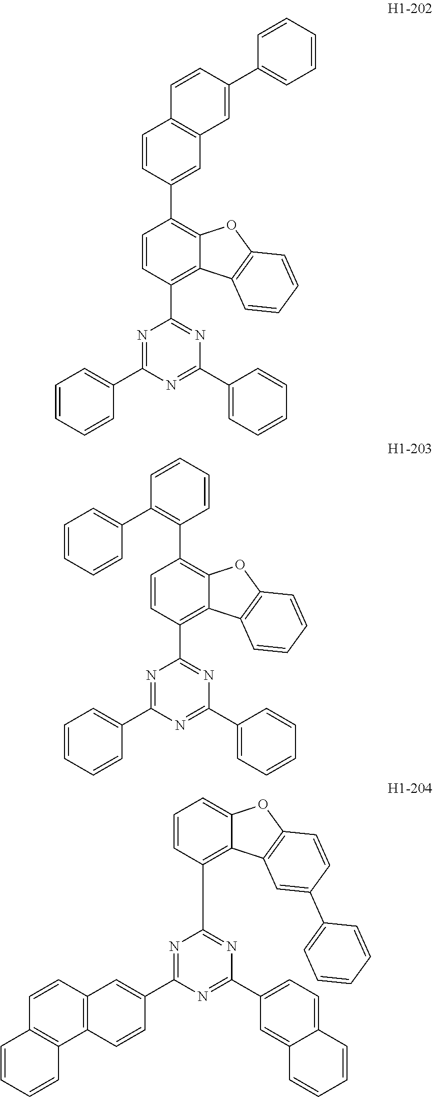

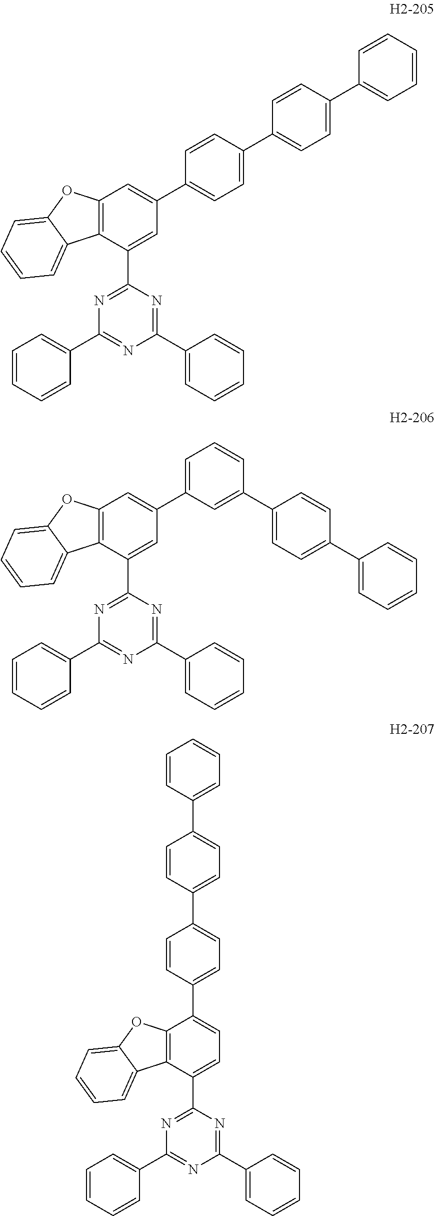

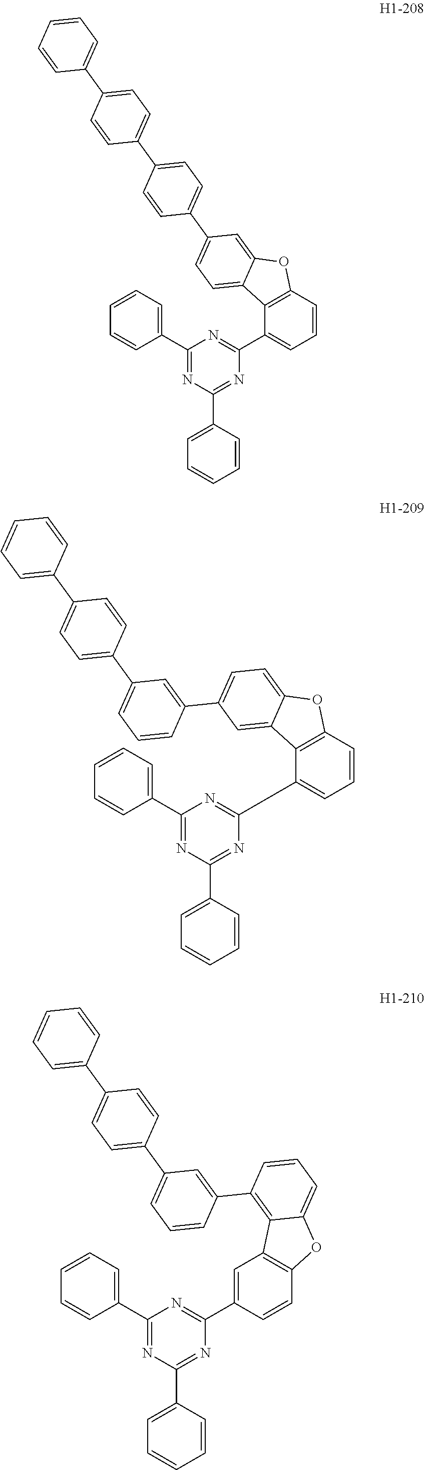 [SMILES: c1ccc(-c2ccc(-c3ccc(-c4ccc5c(c4)oc4cccc(-c6nc(-c7ccccc7)nc(-c7ccccc7)n6)c45)cc3)cc2)cc1 ; c1ccc(-c2ccc(-c3cccc(-c4ccc5oc6cccc(-c7nc(-c8ccccc8)nc(-c8ccccc8)n7)c6c5c4)c3)cc2)cc1 ; c1ccc(-c2ccc(-c3cccc(-c4cccc5oc6ccc(-c7nc(-c8ccccc8)nc(-c8ccccc8)n7)cc6c45)c3)cc2)cc1]

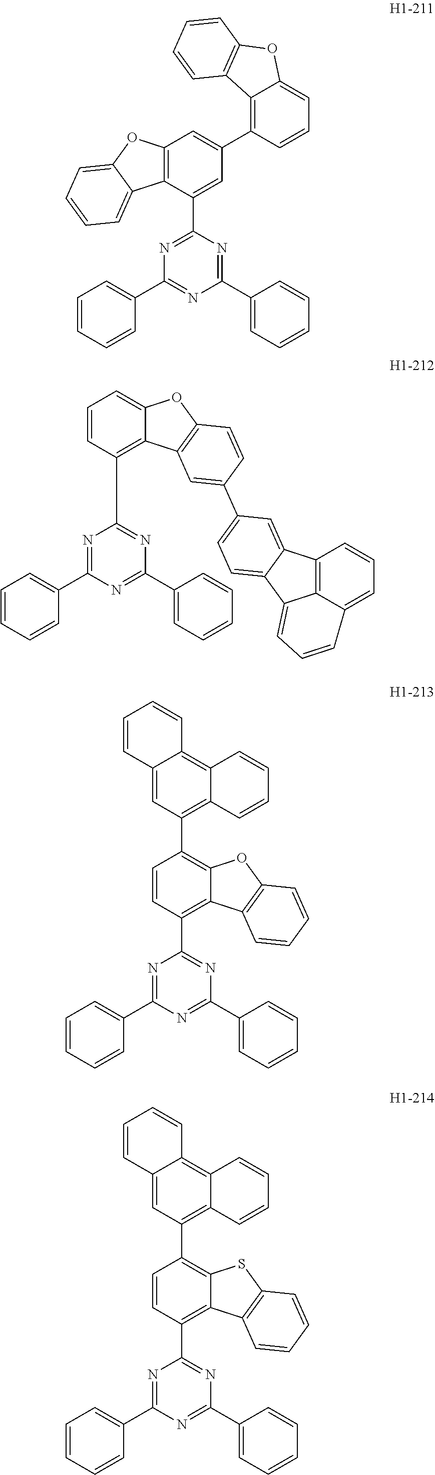

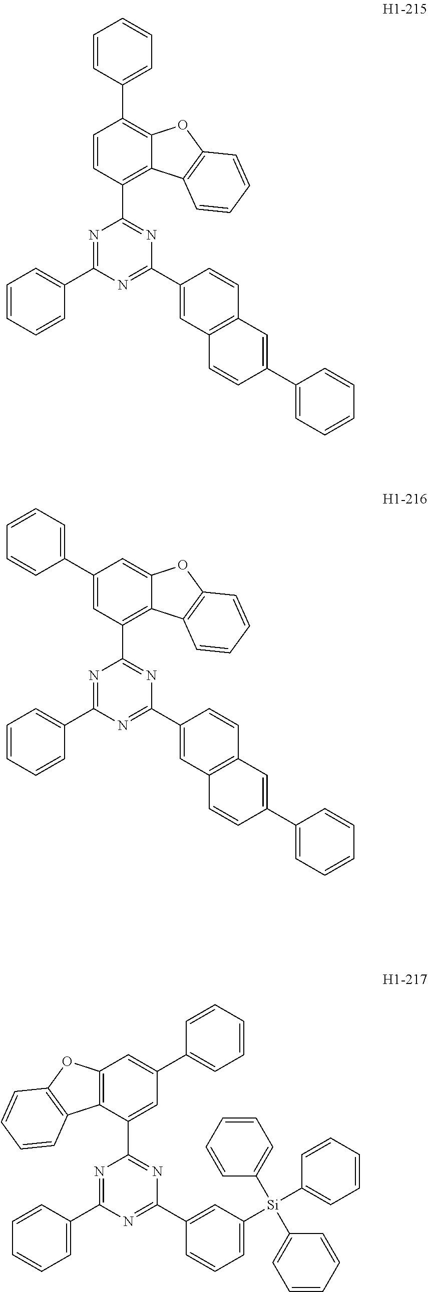 [SMILES: c1ccc(-c2ccc3cc(-c4nc(-c5ccccc5)nc(-c5ccc(-c6ccccc6)c6oc7ccccc7c56)n4)ccc3c2)cc1 ; c1ccc(-c2ccc3cc(-c4nc(-c5ccccc5)nc(-c5cc(-c6ccccc6)cc6oc7ccccc7c56)n4)ccc3c2)cc1 ; c1ccc(-c2cc(-c3nc(-c4ccccc4)nc(-c4cccc([Si](c5ccccc5)(c5ccccc5)c5ccccc5)c4)n3)c3c(c2)oc2ccccc23)cc1]

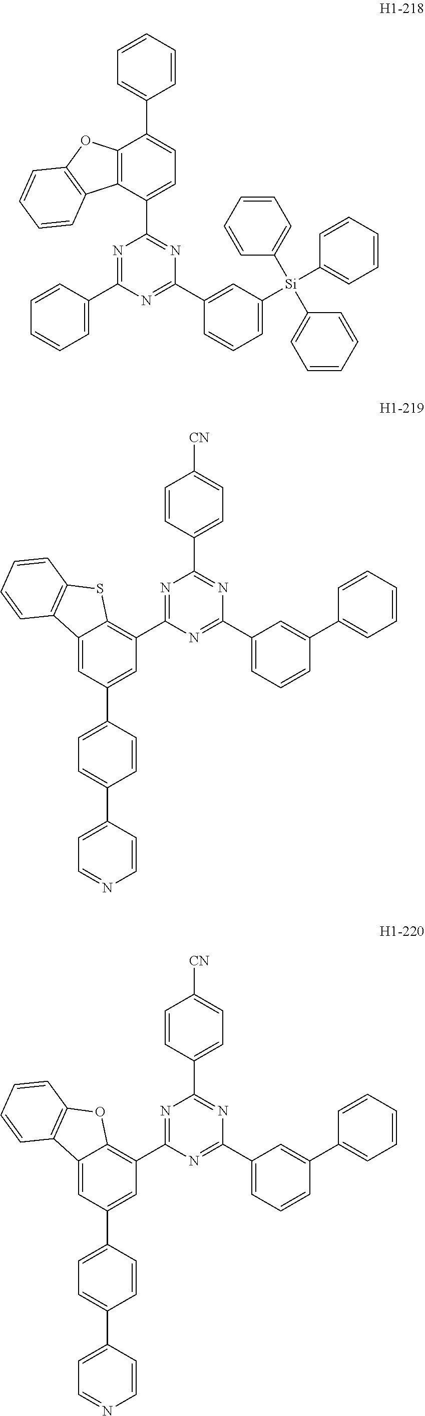 [SMILES: c1ccc(-c2nc(-c3cccc([Si](c4ccccc4)(c4ccccc4)c4ccccc4)c3)nc(-c3ccc(-c4ccccc4)c4oc5ccccc5c34)n2)cc1 ; N#Cc1ccc(-c2nc(-c3cccc(-c4ccccc4)c3)nc(-c3cc(-c4ccc(-c5ccncc5)cc4)cc4c3sc3ccccc34)n2)cc1 ; N#Cc1ccc(-c2nc(-c3cccc(-c4ccccc4)c3)nc(-c3cc(-c4ccc(-c5ccncc5)cc4)cc4c3oc3ccccc34)n2)cc1]

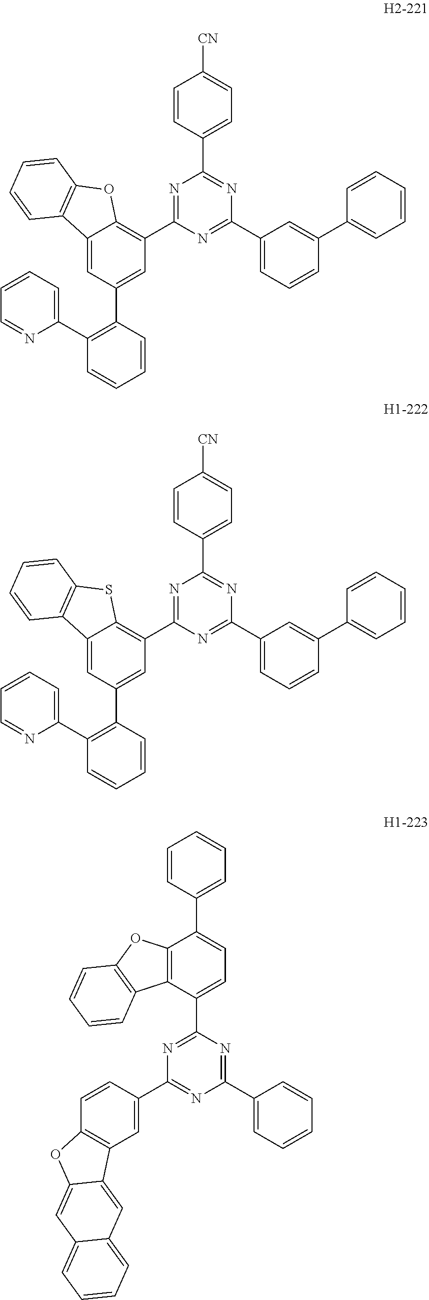 [SMILES: N#Cc1ccc(-c2nc(-c3cccc(-c4ccccc4)c3)nc(-c3cc(-c4ccccc4-c4ccccn4)cc4c3oc3ccccc34)n2)cc1 ; N#Cc1ccc(-c2nc(-c3cccc(-c4ccccc4)c3)nc(-c3cc(-c4ccccc4-c4ccccn4)cc4c3sc3ccccc34)n2)cc1 ; c1ccc(-c2nc(-c3ccc4oc5cc6ccccc6cc5c4c3)nc(-c3ccc(-c4ccccc4)c4oc5ccccc5c34)n2)cc1]

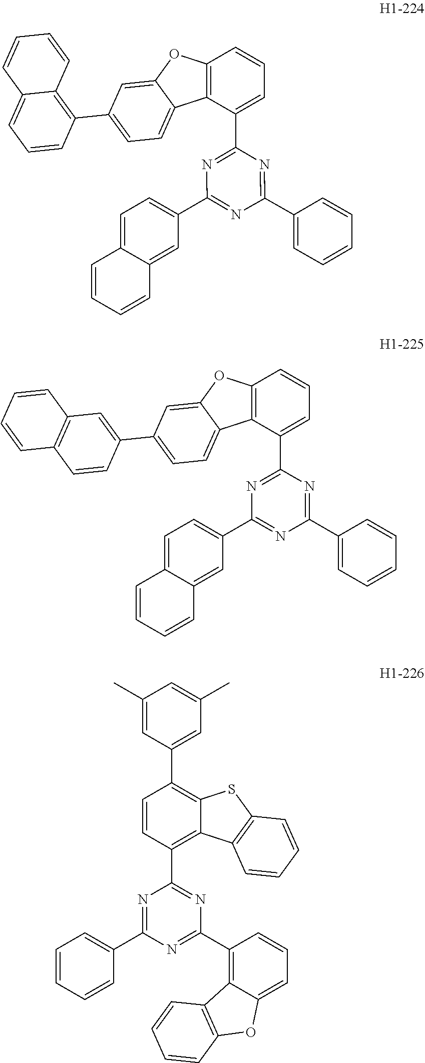

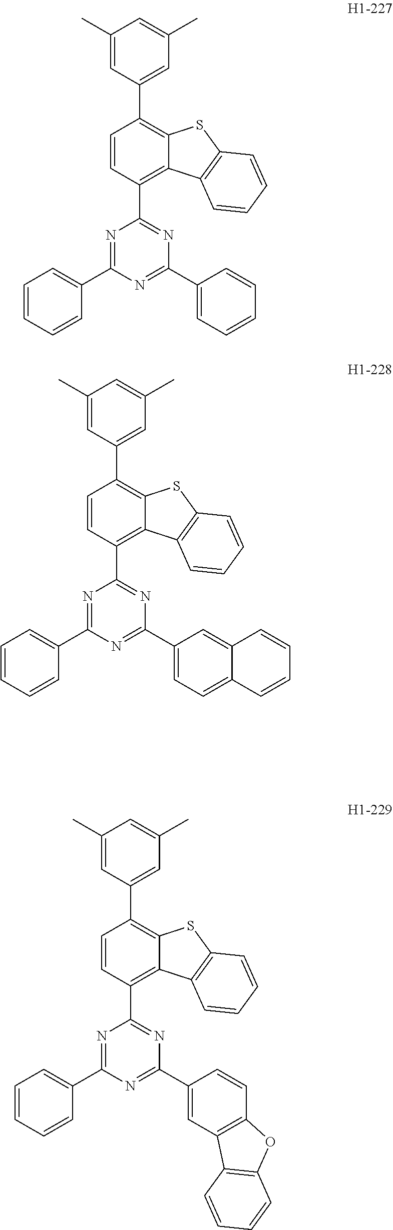

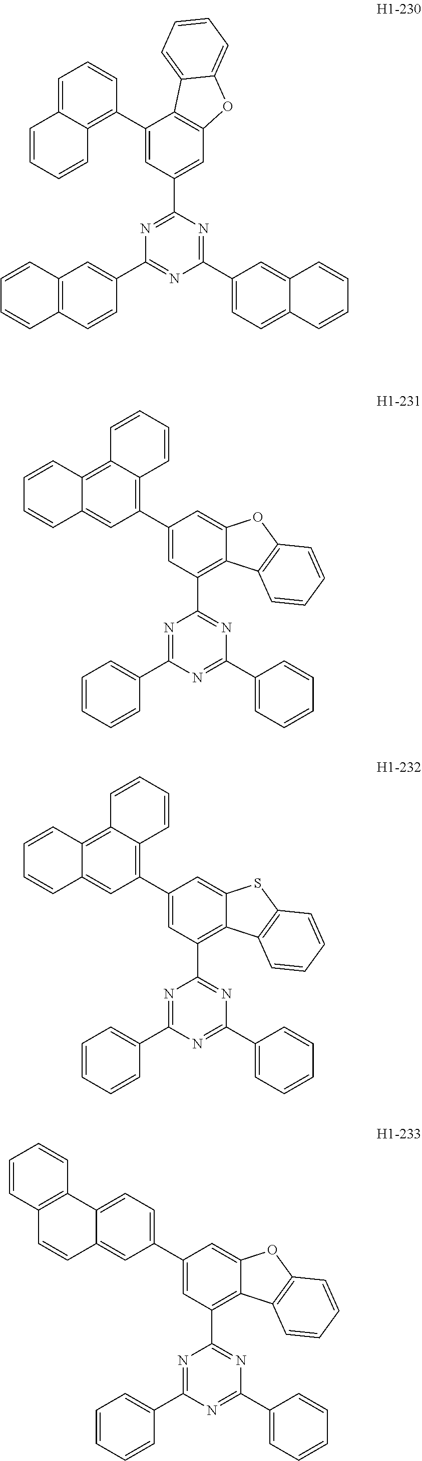 [SMILES: c1ccc2cc(-c3nc(-c4ccc5ccccc5c4)nc(-c4cc(-c5cccc6ccccc56)c5c(c4)oc4ccccc45)n3)ccc2c1 ; c1ccc(-c2nc(-c3ccccc3)nc(-c3cc(-c4cc5ccccc5c5ccccc45)cc4oc5ccccc5c34)n2)cc1 ; c1ccc(-c2nc(-c3ccccc3)nc(-c3cc(-c4cc5ccccc5c5ccccc45)cc4sc5ccccc5c34)n2)cc1 ; c1ccc(-c2nc(-c3ccccc3)nc(-c3cc(-c4ccc5c(ccc6ccccc65)c4)cc4oc5ccccc5c34)n2)cc1]

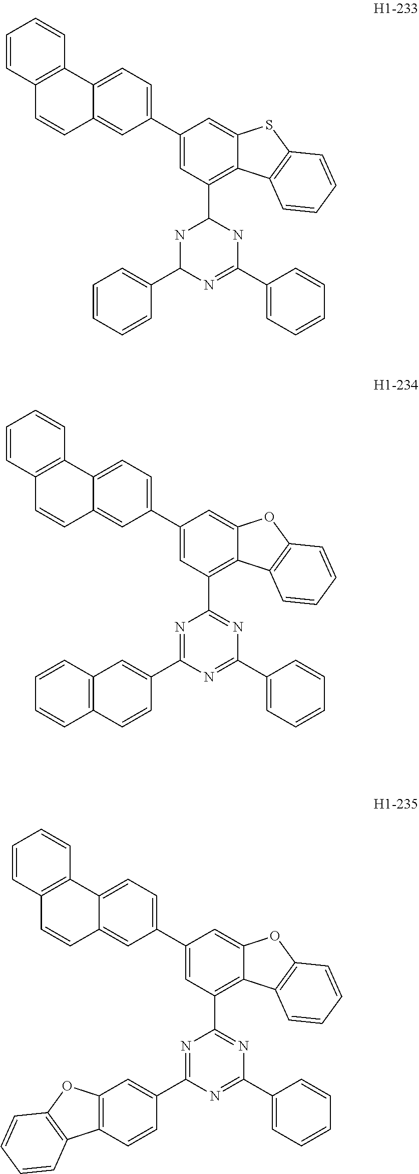

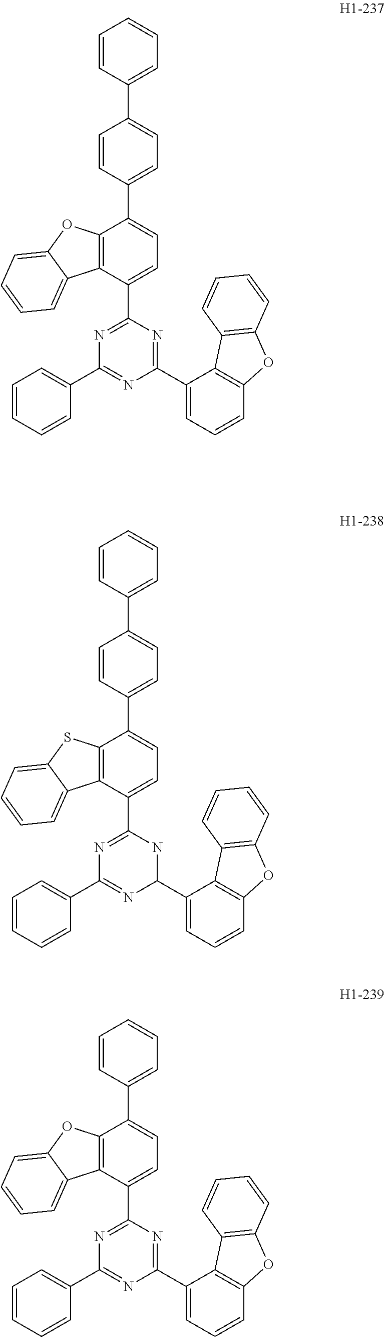 [SMILES: c1ccc(-c2ccc(-c3ccc(-c4nc(-c5ccccc5)nc(-c5cccc6oc7ccccc7c56)n4)c4c3oc3ccccc34)cc2)cc1 ; c1ccc(C2=NC(c3cccc4oc5ccccc5c34)NC(c3ccc(-c4ccc(-c5ccccc5)cc4)c4sc5ccccc5c34)=N2)cc1 ; c1ccc(-c2nc(-c3cccc4oc5ccccc5c34)nc(-c3ccc(-c4ccccc4)c4oc5ccccc5c34)n2)cc1]

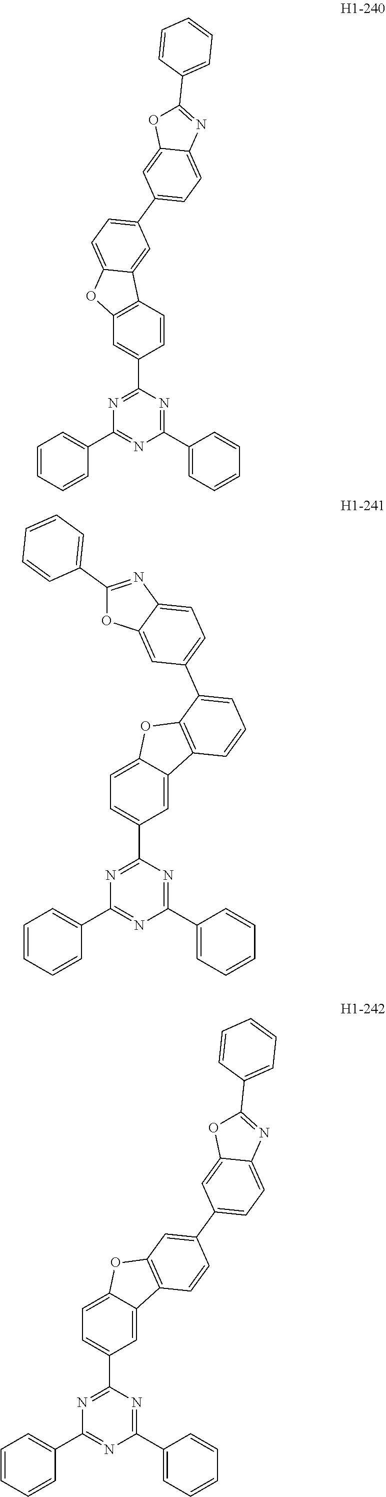

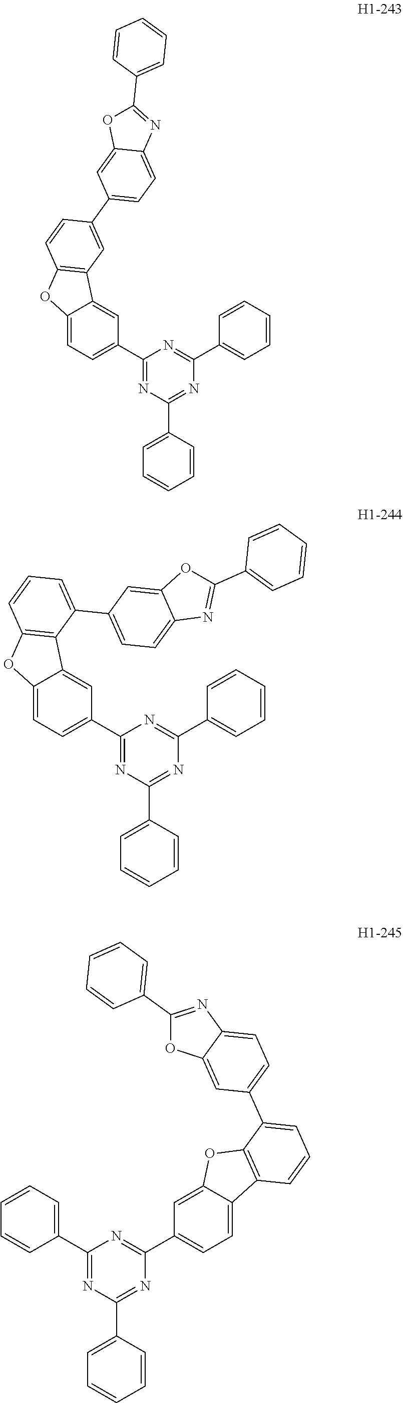 [SMILES: c1ccc(-c2nc(-c3ccccc3)nc(-c3ccc4oc5ccc(-c6ccc7nc(-c8ccccc8)oc7c6)cc5c4c3)n2)cc1 ; c1ccc(-c2nc(-c3ccccc3)nc(-c3ccc4oc5cccc(-c6ccc7nc(-c8ccccc8)oc7c6)c5c4c3)n2)cc1 ; c1ccc(-c2nc(-c3ccccc3)nc(-c3ccc4c(c3)oc3c(-c5ccc6nc(-c7ccccc7)oc6c5)cccc34)n2)cc1]

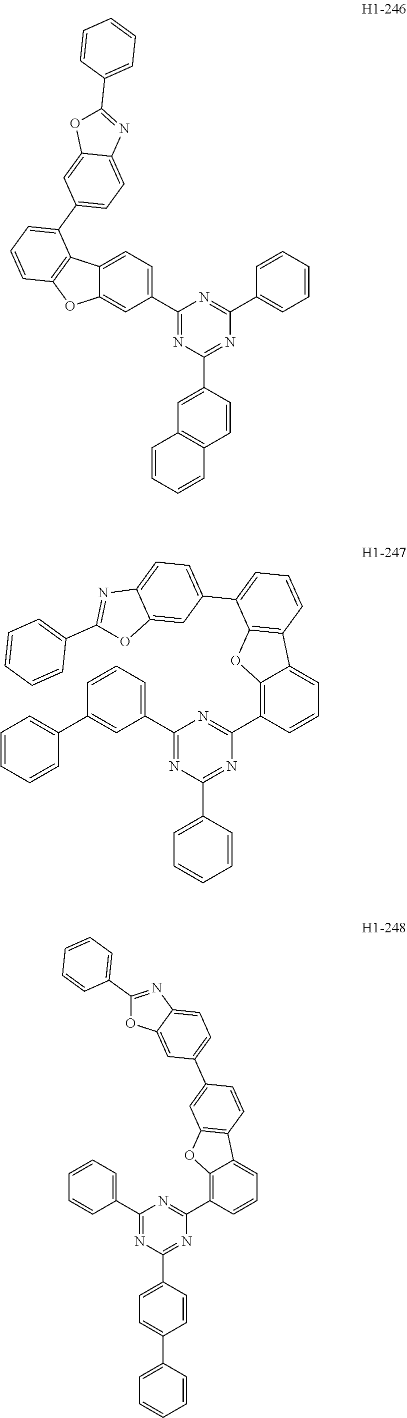 [SMILES: c1ccc(-c2nc(-c3ccc4ccccc4c3)nc(-c3ccc4c(c3)oc3cccc(-c5ccc6nc(-c7ccccc7)oc6c5)c34)n2)cc1 ; c1ccc(-c2cccc(-c3nc(-c4ccccc4)nc(-c4cccc5c4oc4c(-c6ccc7nc(-c8ccccc8)oc7c6)cccc45)n3)c2)cc1 ; c1ccc(-c2ccc(-c3nc(-c4ccccc4)nc(-c4cccc5c4oc4cc(-c6ccc7nc(-c8ccccc8)oc7c6)ccc45)n3)cc2)cc1]

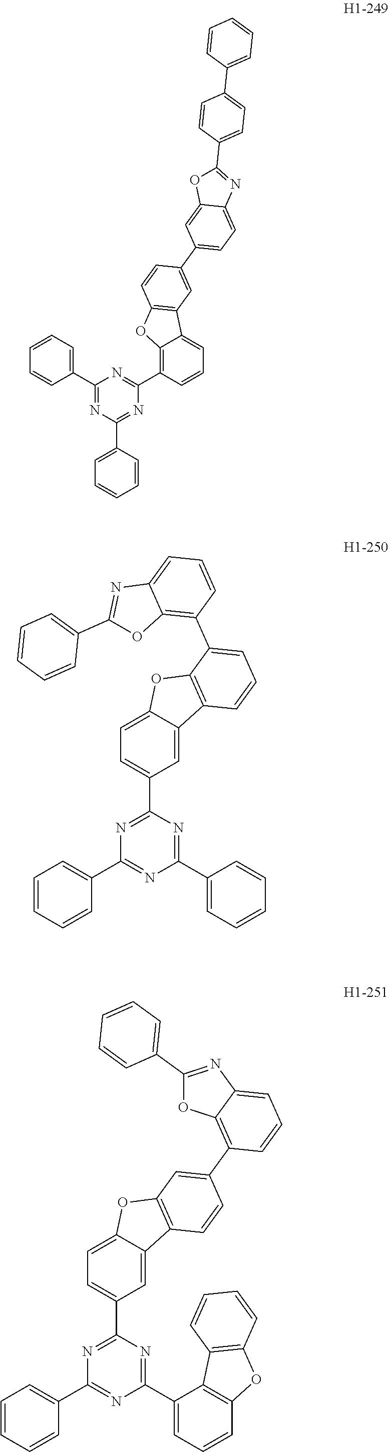 [SMILES: c1ccc(-c2ccc(-c3nc4ccc(-c5ccc6oc7c(-c8nc(-c9ccccc9)nc(-c9ccccc9)n8)cccc7c6c5)cc4o3)cc2)cc1 ; c1ccc(-c2nc(-c3ccccc3)nc(-c3ccc4oc5c(-c6cccc7nc(-c8ccccc8)oc67)cccc5c4c3)n2)cc1 ; c1ccc(-c2nc(-c3ccc4oc5cc(-c6cccc7nc(-c8ccccc8)oc67)ccc5c4c3)nc(-c3cccc4oc5ccccc5c34)n2)cc1]

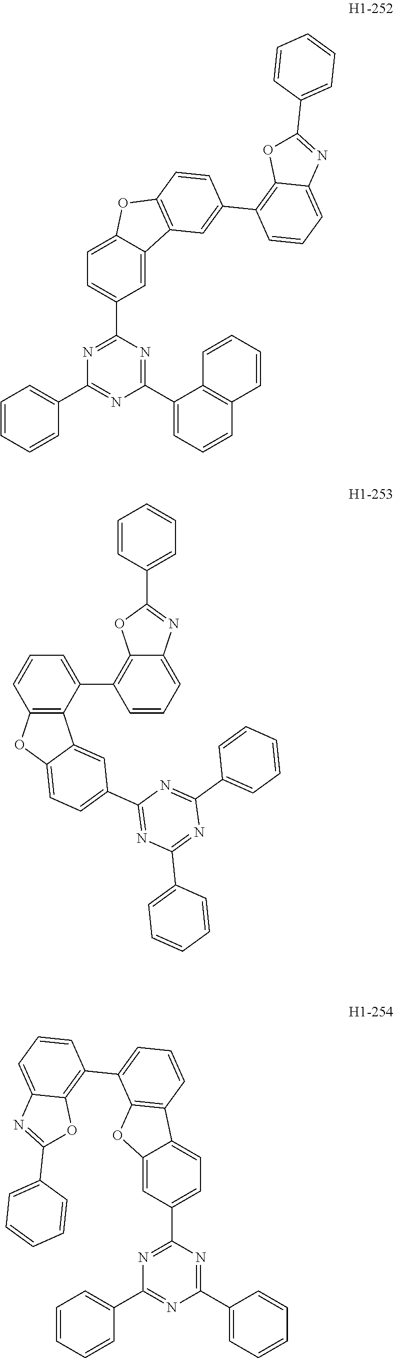 [SMILES: c1ccc(-c2nc(-c3ccc4oc5ccc(-c6cccc7nc(-c8ccccc8)oc67)cc5c4c3)nc(-c3cccc4ccccc34)n2)cc1 ; c1ccc(-c2nc(-c3ccccc3)nc(-c3ccc4oc5cccc(-c6cccc7nc(-c8ccccc8)oc67)c5c4c3)n2)cc1 ; c1ccc(-c2nc(-c3ccccc3)nc(-c3ccc4c(c3)oc3c(-c5cccc6nc(-c7ccccc7)oc56)cccc34)n2)cc1]

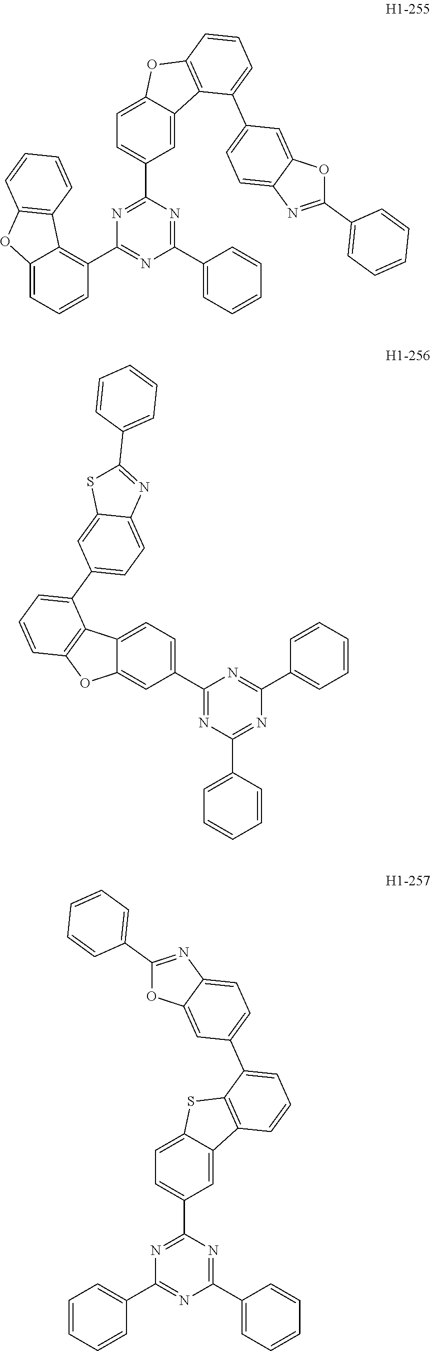 [SMILES: c1ccc(-c2nc(-c3ccc4oc5cccc(-c6ccc7nc(-c8ccccc8)oc7c6)c5c4c3)nc(-c3cccc4oc5ccccc5c34)n2)cc1 ; c1ccc(-c2nc(-c3ccccc3)nc(-c3ccc4c(c3)oc3cccc(-c5ccc6nc(-c7ccccc7)sc6c5)c34)n2)cc1 ; c1ccc(-c2nc(-c3ccccc3)nc(-c3ccc4sc5c(-c6ccc7nc(-c8ccccc8)oc7c6)cccc5c4c3)n2)cc1]

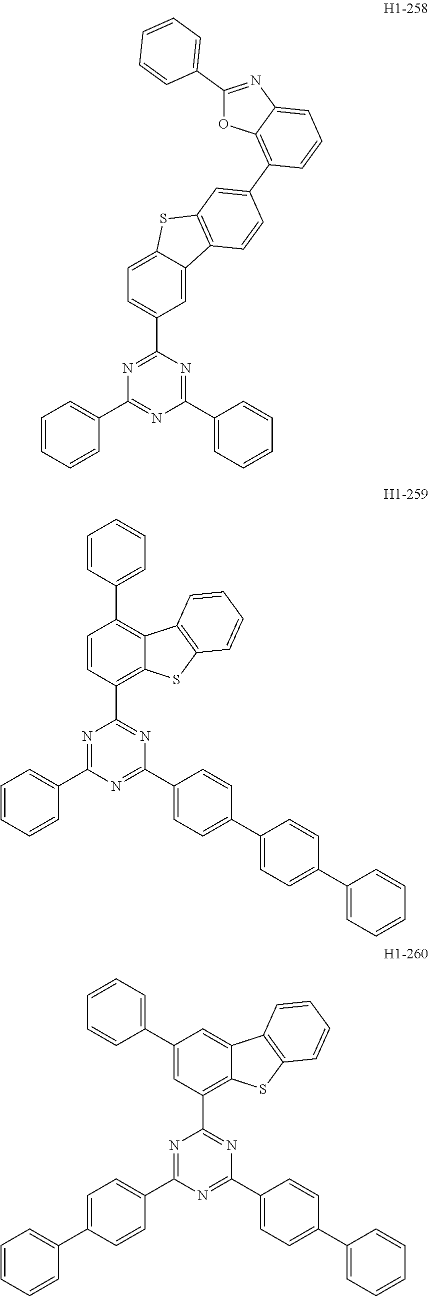

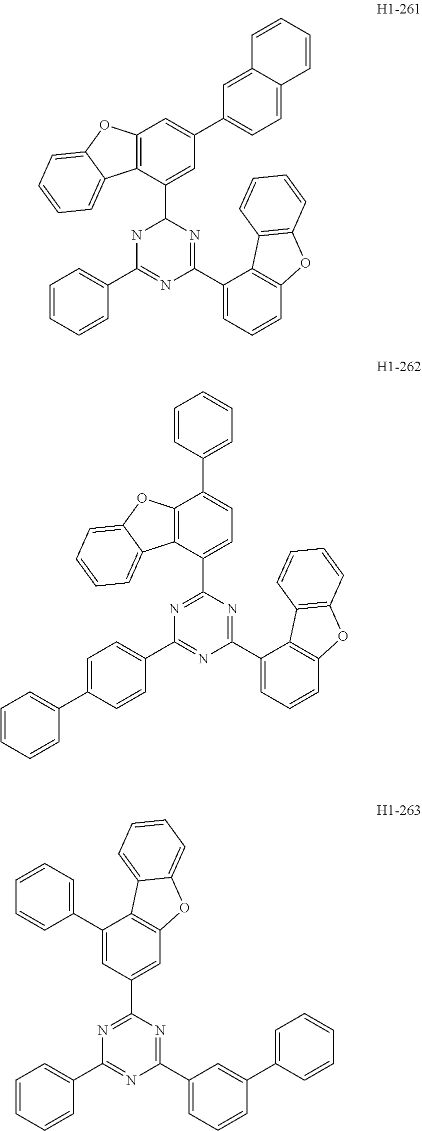

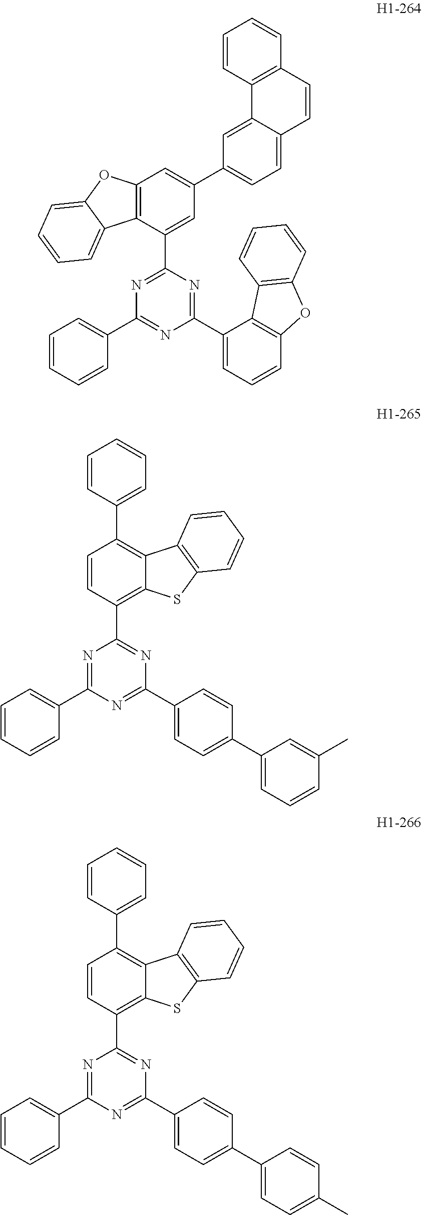 [SMILES: c1ccc(-c2nc(-c3cccc4oc5ccccc5c34)nc(-c3cc(-c4ccc5ccc6ccccc6c5c4)cc4oc5ccccc5c34)n2)cc1 ; Cc1cccc(-c2ccc(-c3nc(-c4ccccc4)nc(-c4ccc(-c5ccccc5)c5c4sc4ccccc45)n3)cc2)c1 ; Cc1ccc(-c2ccc(-c3nc(-c4ccccc4)nc(-c4ccc(-c5ccccc5)c5c4sc4ccccc45)n3)cc2)cc1]

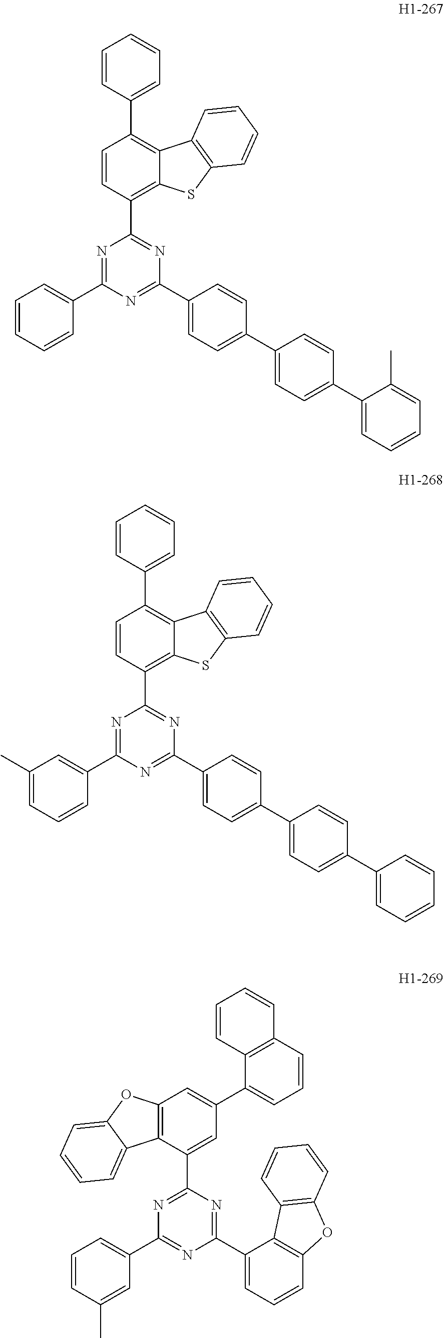 [SMILES: Cc1ccccc1-c1ccc(-c2ccc(-c3nc(-c4ccccc4)nc(-c4ccc(-c5ccccc5)c5c4sc4ccccc45)n3)cc2)cc1 ; Cc1cccc(-c2nc(-c3ccc(-c4ccc(-c5ccccc5)cc4)cc3)nc(-c3ccc(-c4ccccc4)c4c3sc3ccccc34)n2)c1 ; Cc1cccc(-c2nc(-c3cccc4oc5ccccc5c34)nc(-c3cc(-c4cccc5ccccc45)cc4oc5ccccc5c34)n2)c1]

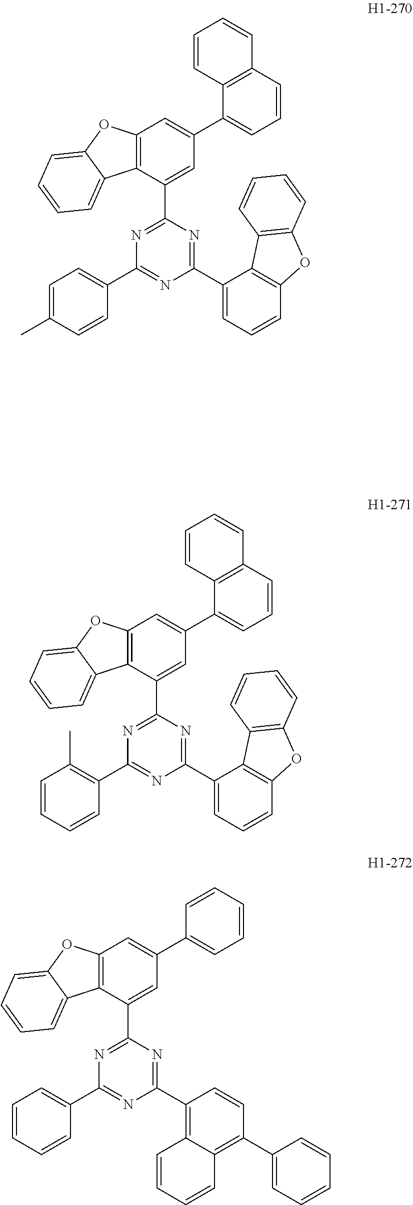

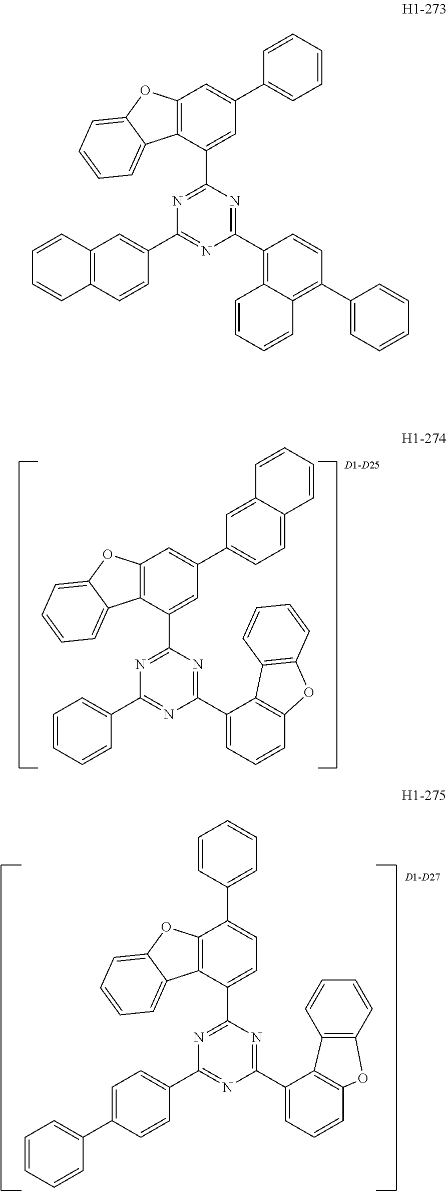

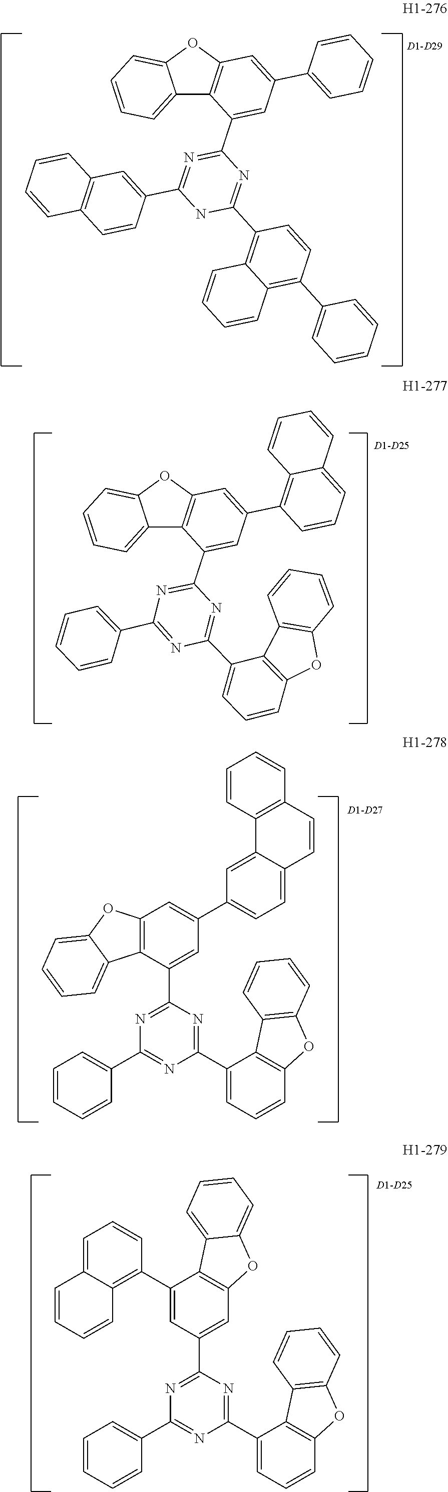

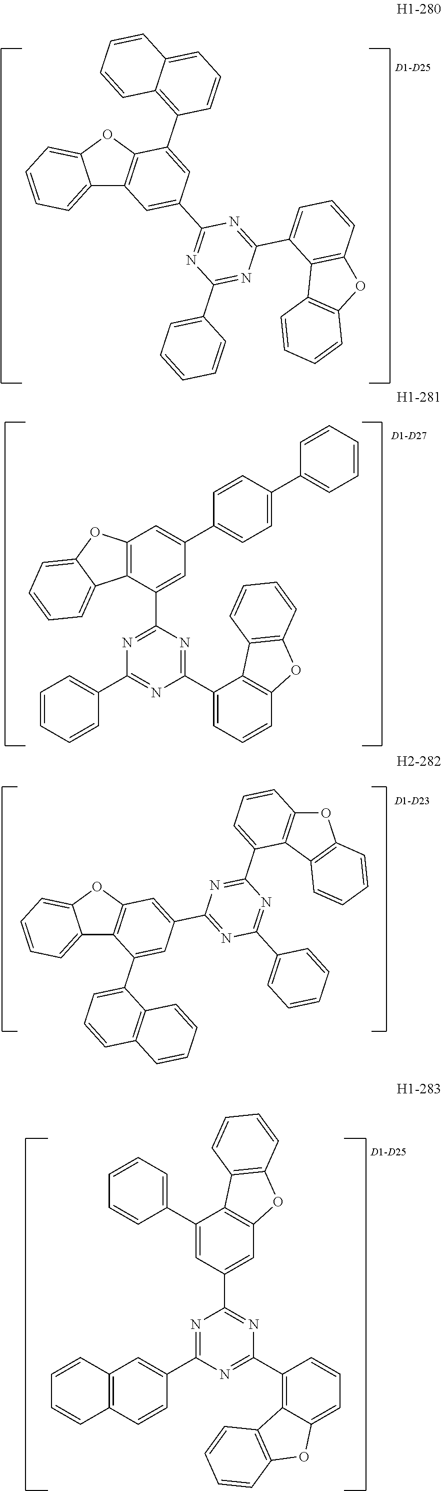

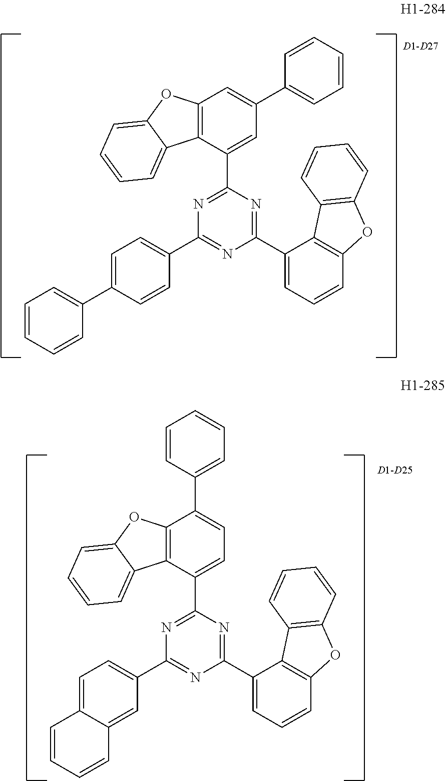

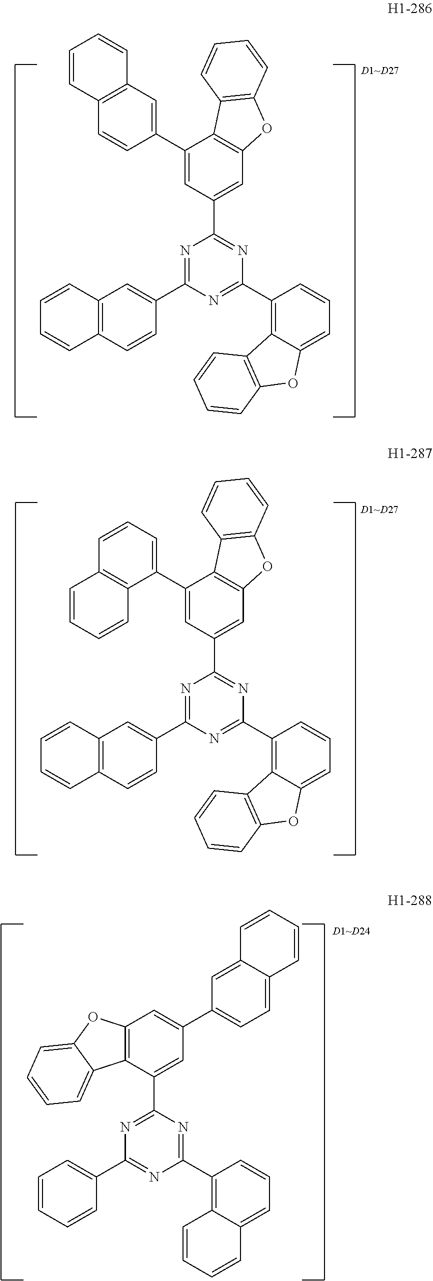

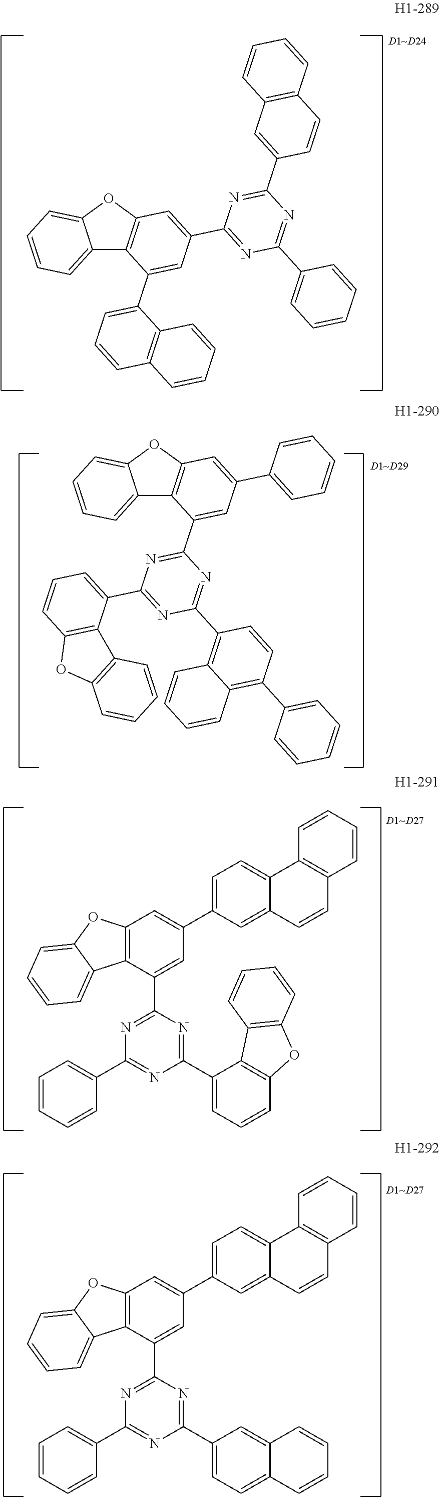

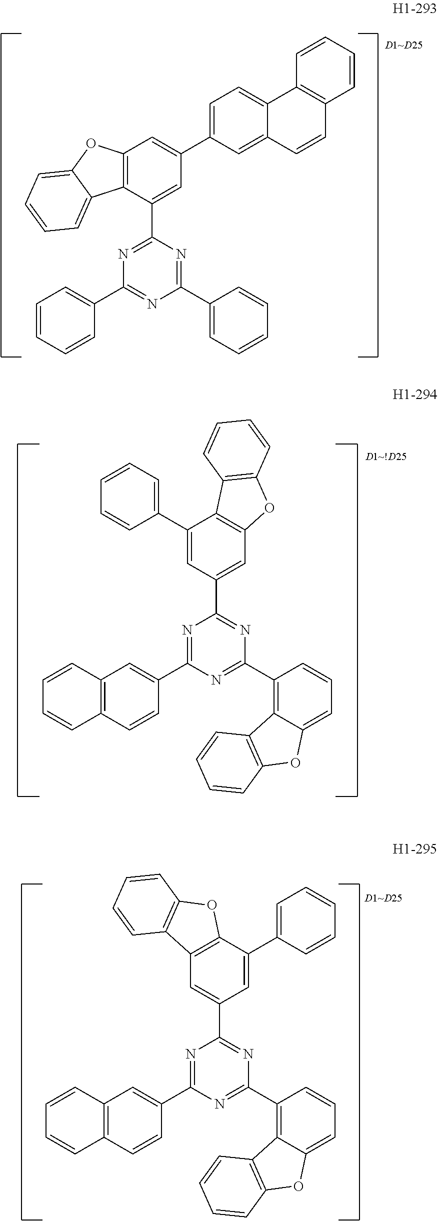

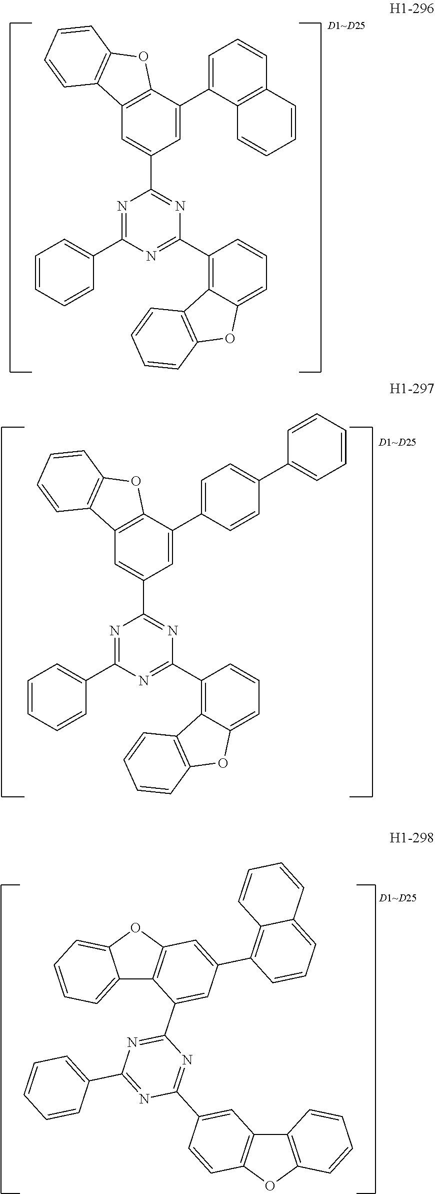

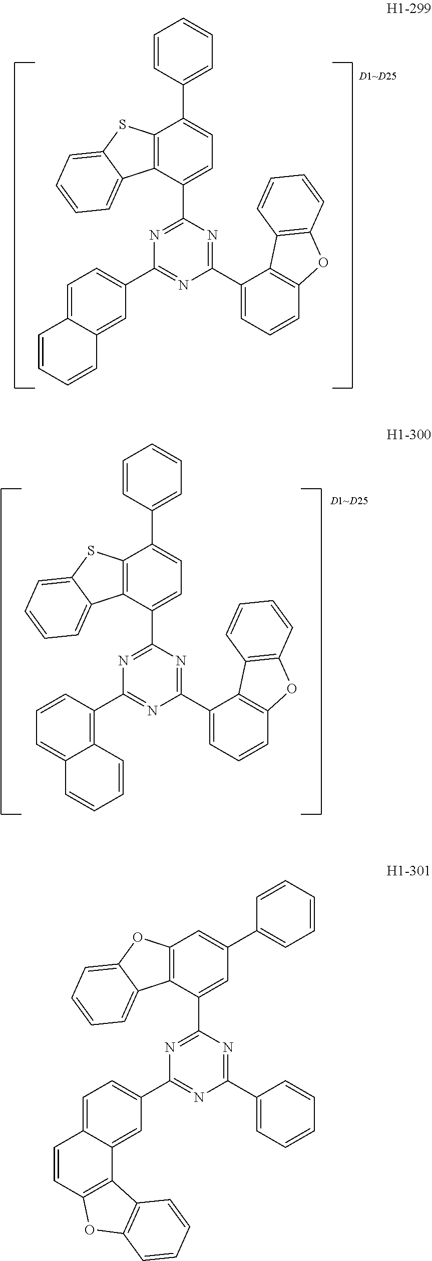

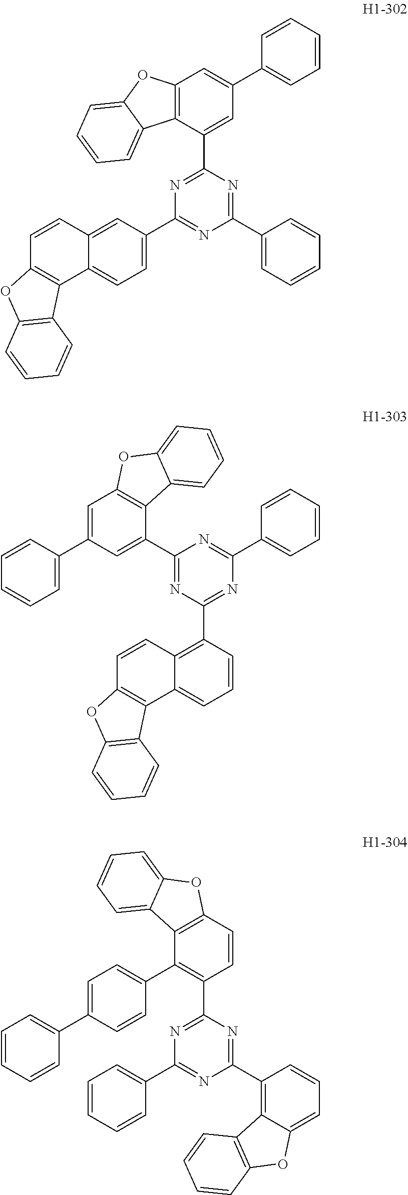

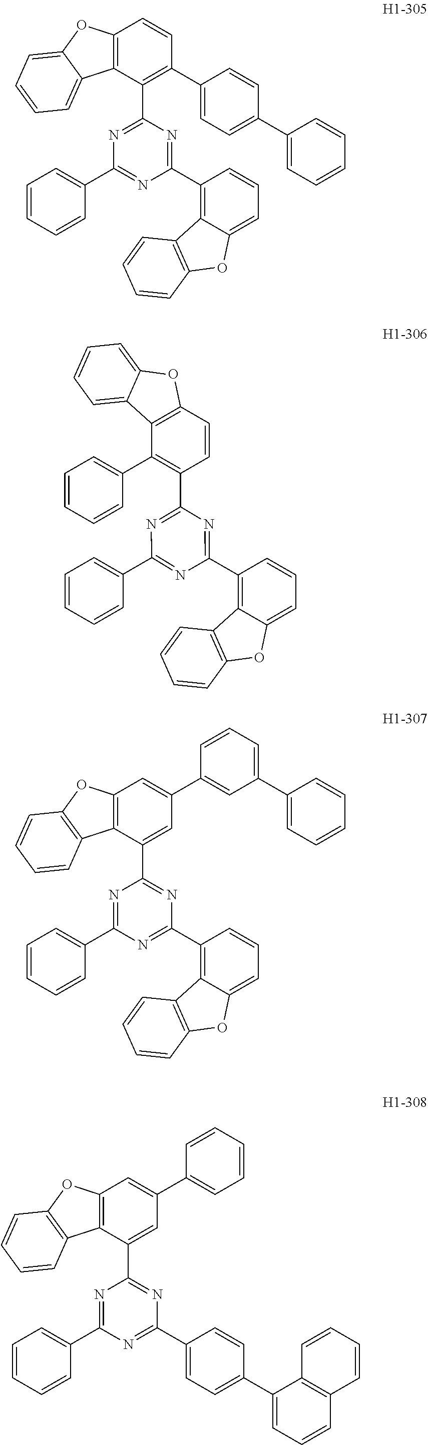

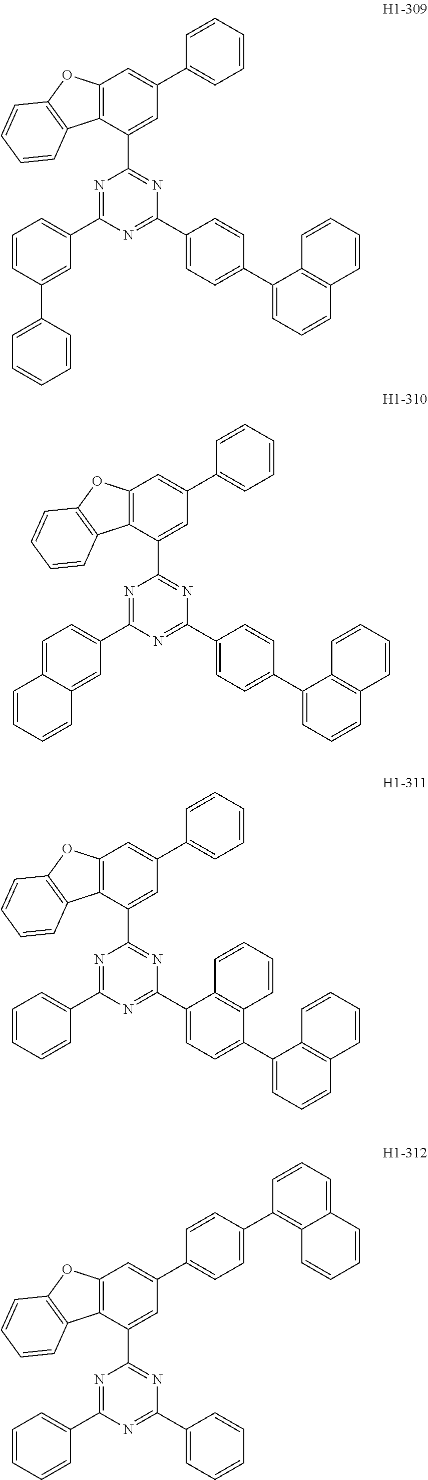 [SMILES: c1ccc(-c2cccc(-c3nc(-c4ccc(-c5cccc6ccccc56)cc4)nc(-c4cc(-c5ccccc5)cc5oc6ccccc6c45)n3)c2)cc1 ; c1ccc(-c2cc(-c3nc(-c4ccc(-c5cccc6ccccc56)cc4)nc(-c4ccc5ccccc5c4)n3)c3c(c2)oc2ccccc23)cc1 ; c1ccc(-c2cc(-c3nc(-c4ccccc4)nc(-c4ccc(-c5cccc6ccccc56)c5ccccc45)n3)c3c(c2)oc2ccccc23)cc1 ; c1ccc(-c2nc(-c3ccccc3)nc(-c3cc(-c4ccc(-c5cccc6ccccc56)cc4)cc4oc5ccccc5c34)n2)cc1]

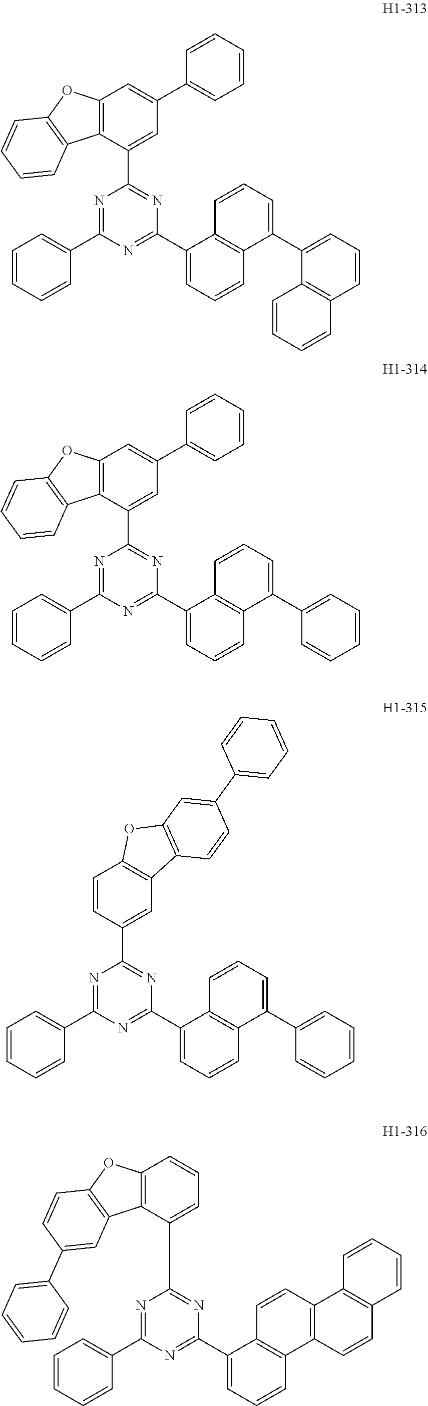 [SMILES: c1ccc(-c2cc(-c3nc(-c4ccccc4)nc(-c4cccc5c(-c6cccc7ccccc67)cccc45)n3)c3c(c2)oc2ccccc23)cc1 ; c1ccc(-c2cc(-c3nc(-c4ccccc4)nc(-c4cccc5c(-c6ccccc6)cccc45)n3)c3c(c2)oc2ccccc23)cc1 ; c1ccc(-c2ccc3c(c2)oc2ccc(-c4nc(-c5ccccc5)nc(-c5cccc6c(-c7ccccc7)cccc56)n4)cc23)cc1 ; c1ccc(-c2ccc3oc4cccc(-c5nc(-c6ccccc6)nc(-c6cccc7c6ccc6c8ccccc8ccc76)n5)c4c3c2)cc1]

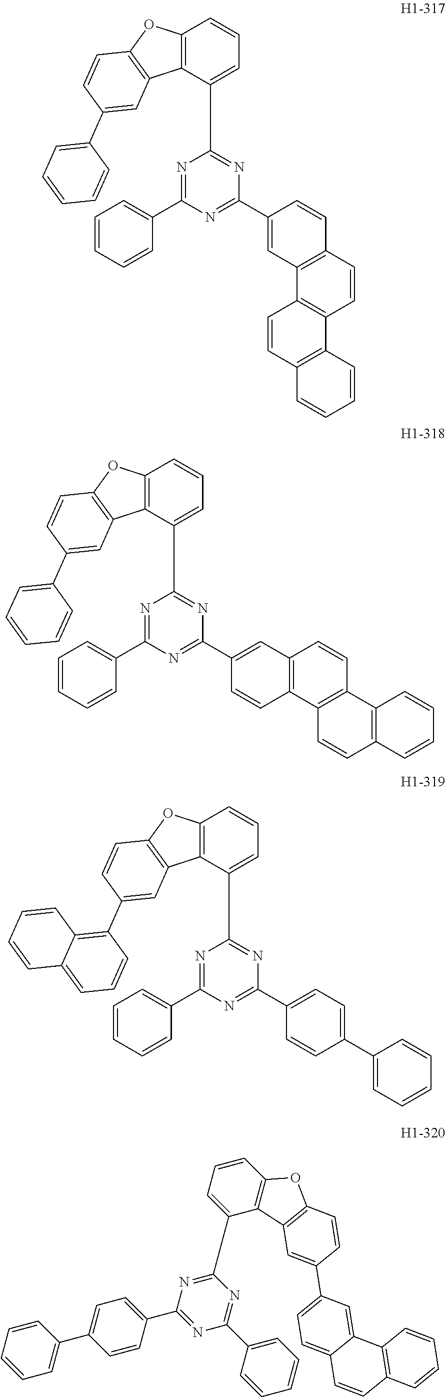 [SMILES: c1ccc(-c2ccc3oc4cccc(-c5nc(-c6ccccc6)nc(-c6ccc7ccc8c9ccccc9ccc8c7c6)n5)c4c3c2)cc1 ; c1ccc(-c2ccc3oc4cccc(-c5nc(-c6ccccc6)nc(-c6ccc7c(ccc8c9ccccc9ccc78)c6)n5)c4c3c2)cc1 ; c1ccc(-c2ccc(-c3nc(-c4ccccc4)nc(-c4cccc5oc6ccc(-c7cccc8ccccc78)cc6c45)n3)cc2)cc1 ; c1ccc(-c2ccc(-c3nc(-c4ccccc4)nc(-c4cccc5oc6ccc(-c7ccc8ccc9ccccc9c8c7)cc6c45)n3)cc2)cc1]

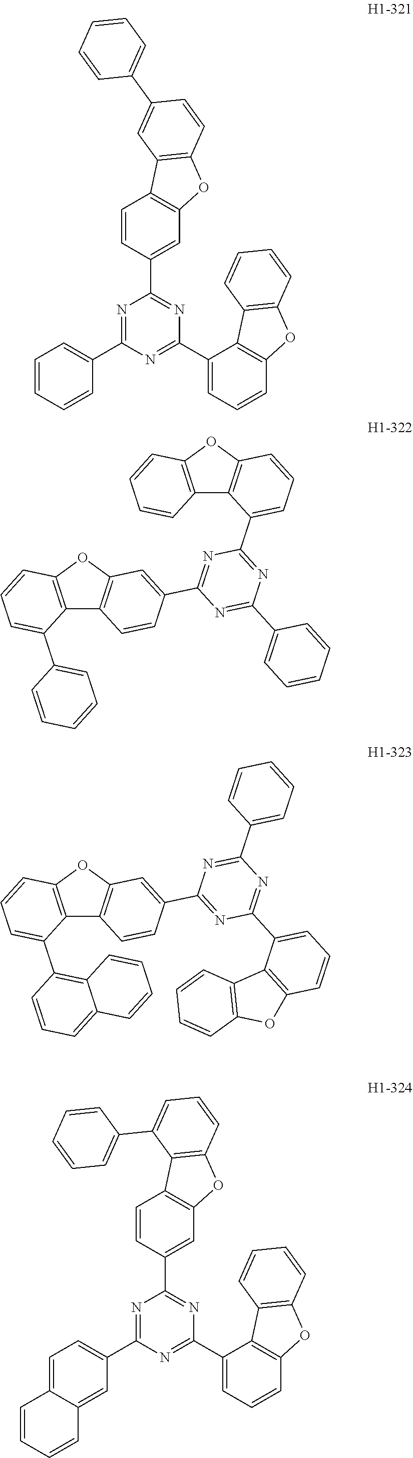

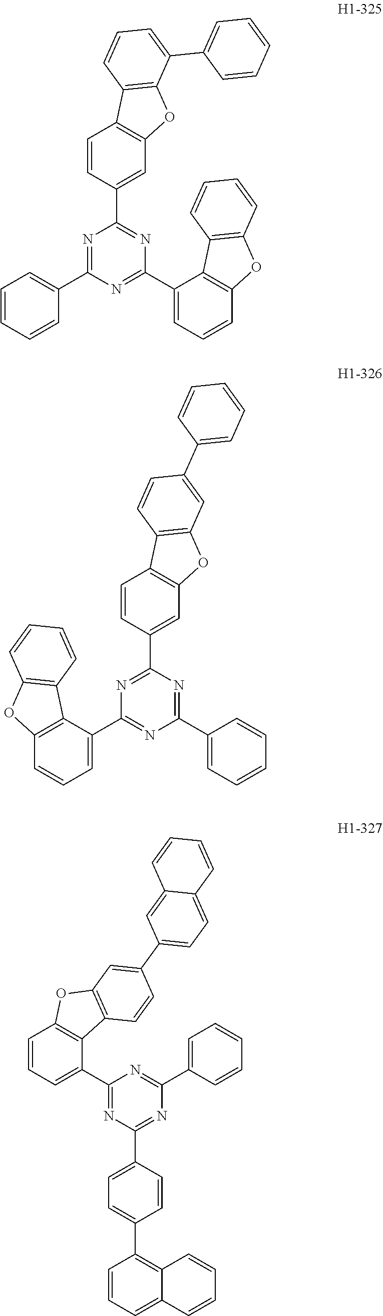

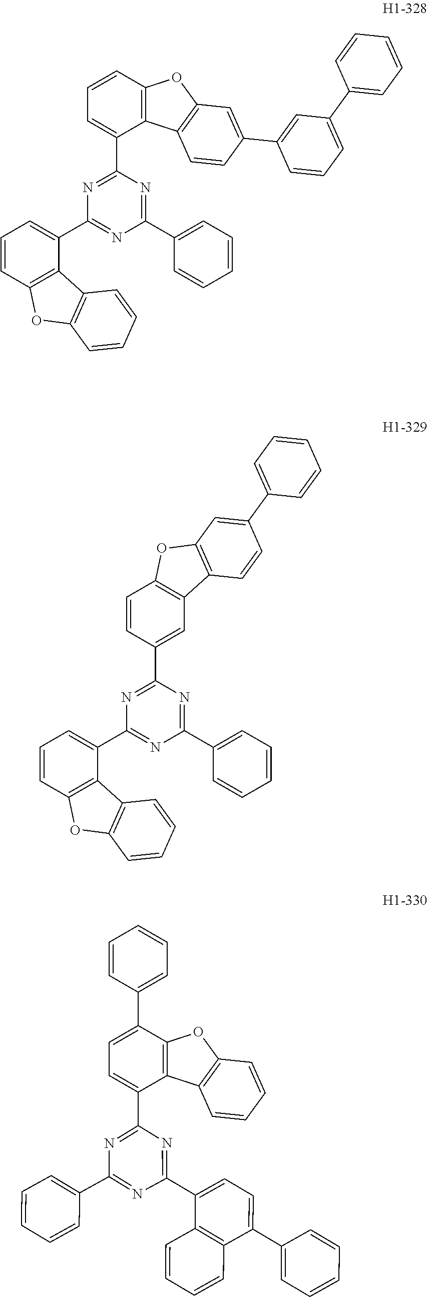

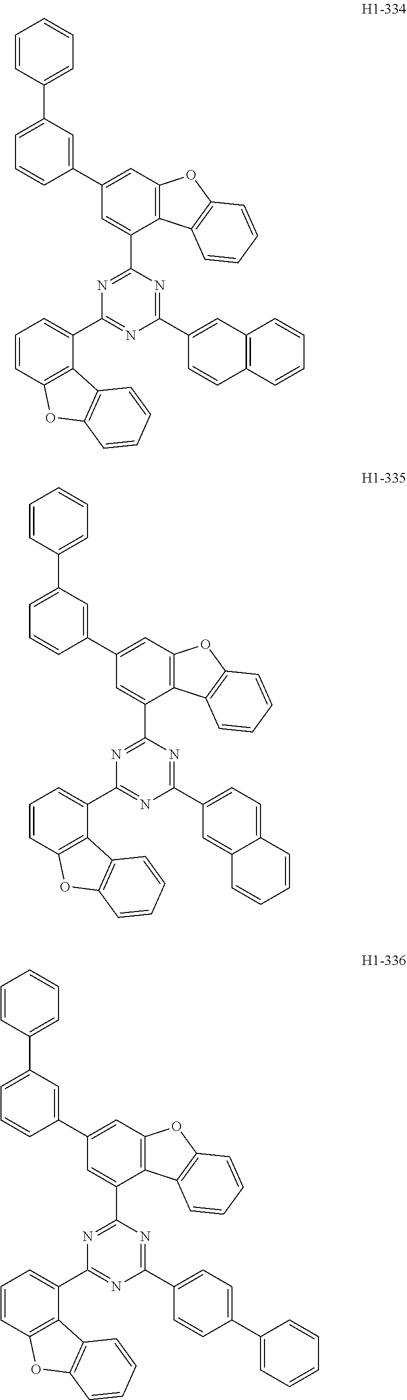

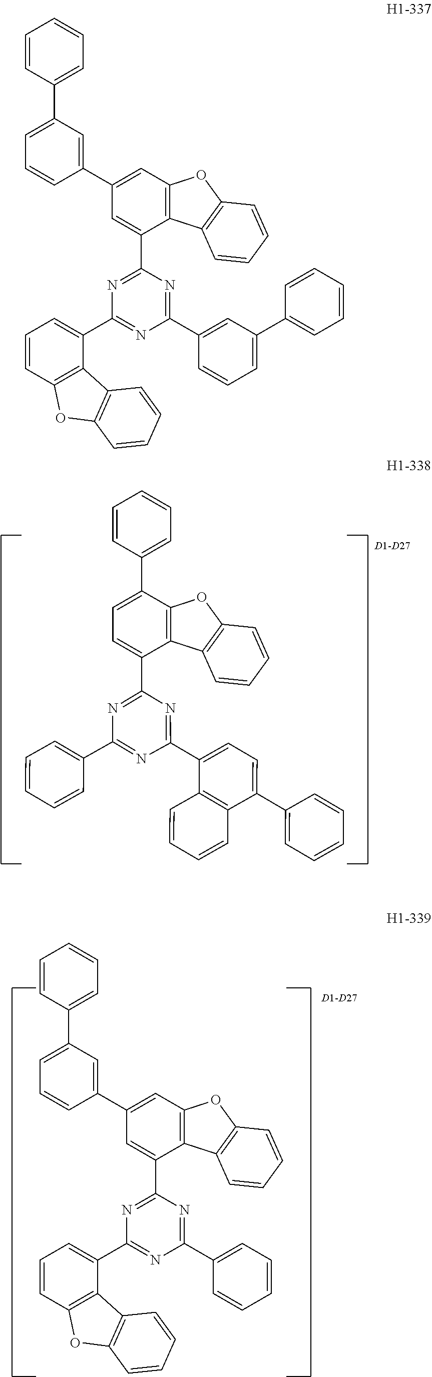

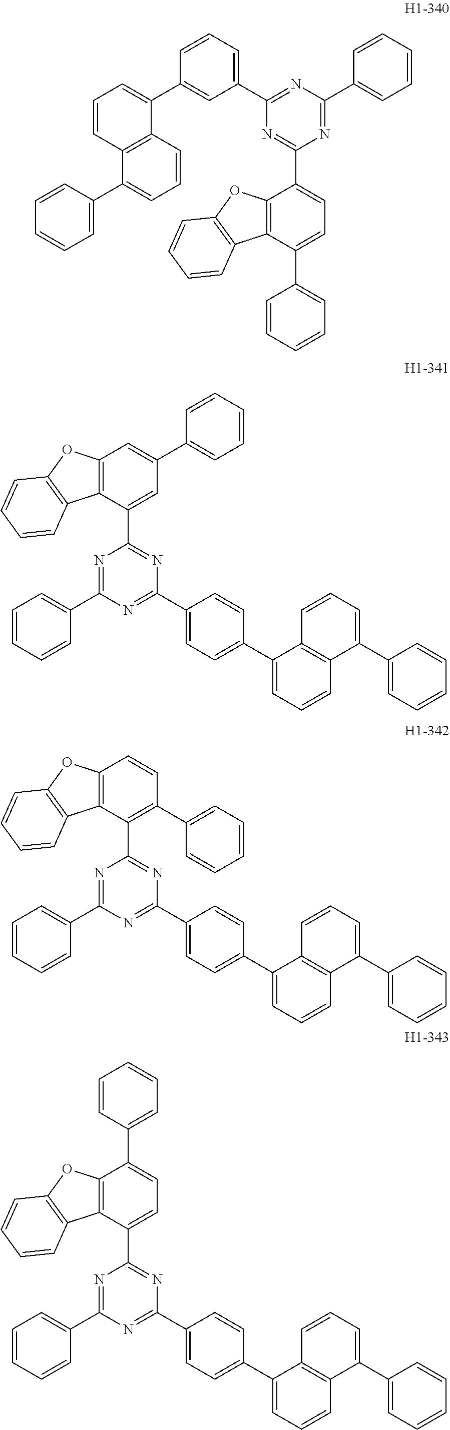

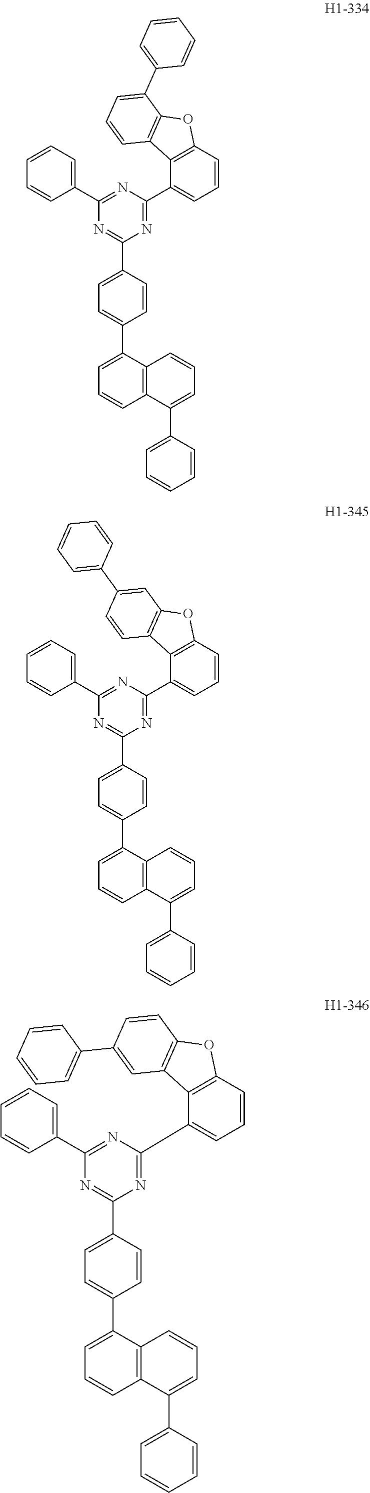

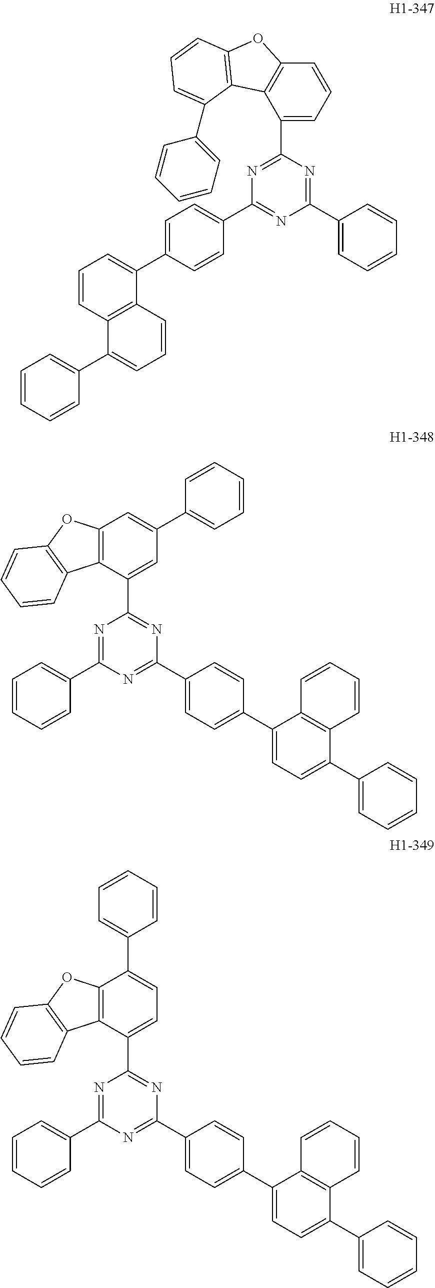

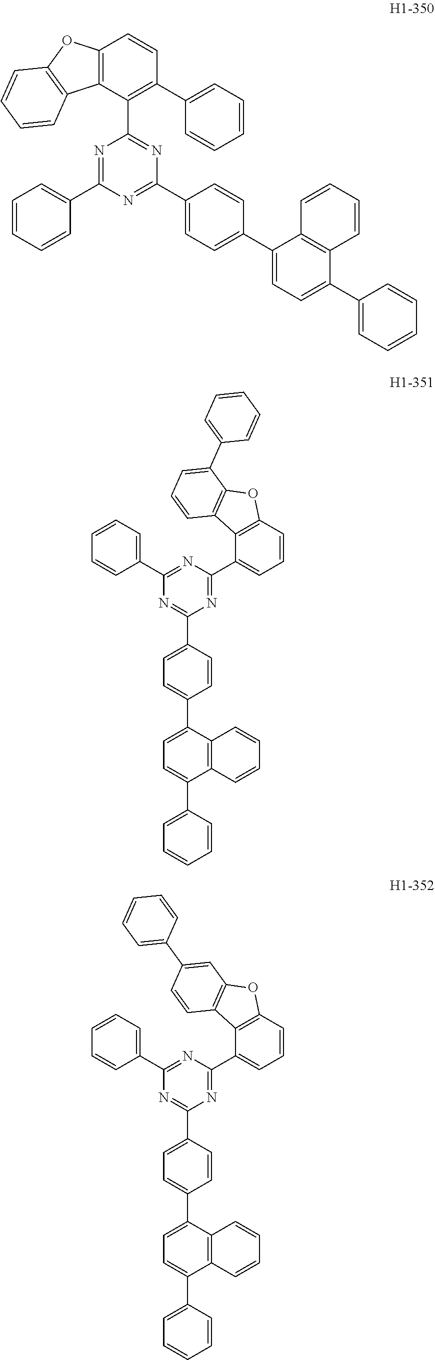 [SMILES: c1ccc(-c2nc(-c3ccc(-c4ccc(-c5ccccc5)c5ccccc45)cc3)nc(-c3c(-c4ccccc4)ccc4oc5ccccc5c34)n2)cc1 ; c1ccc(-c2nc(-c3ccc(-c4ccc(-c5ccccc5)c5ccccc45)cc3)nc(-c3cccc4oc5c(-c6ccccc6)cccc5c34)n2)cc1 ; c1ccc(-c2ccc3c(c2)oc2cccc(-c4nc(-c5ccccc5)nc(-c5ccc(-c6ccc(-c7ccccc7)c7ccccc67)cc5)n4)c23)cc1]

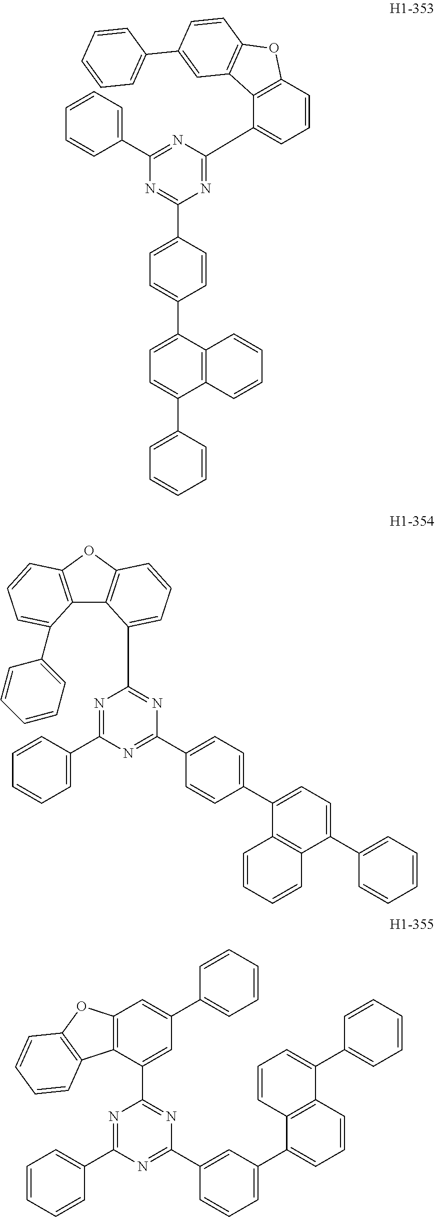

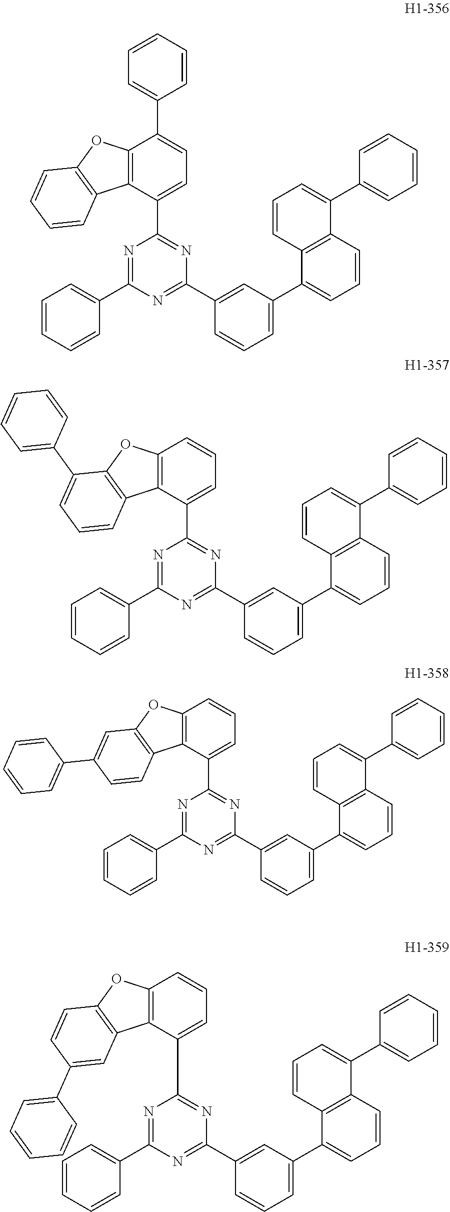

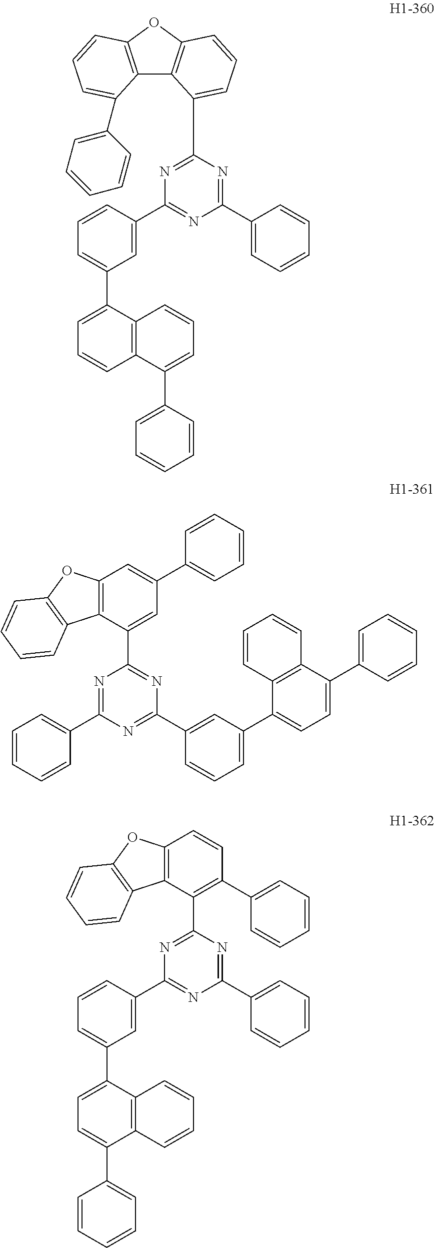 [SMILES: c1ccc(-c2nc(-c3cccc(-c4cccc5c(-c6ccccc6)cccc45)c3)nc(-c3cccc4oc5cccc(-c6ccccc6)c5c34)n2)cc1 ; c1ccc(-c2cc(-c3nc(-c4ccccc4)nc(-c4cccc(-c5ccc(-c6ccccc6)c6ccccc56)c4)n3)c3c(c2)oc2ccccc23)cc1 ; c1ccc(-c2nc(-c3cccc(-c4ccc(-c5ccccc5)c5ccccc45)c3)nc(-c3c(-c4ccccc4)ccc4oc5ccccc5c34)n2)cc1]

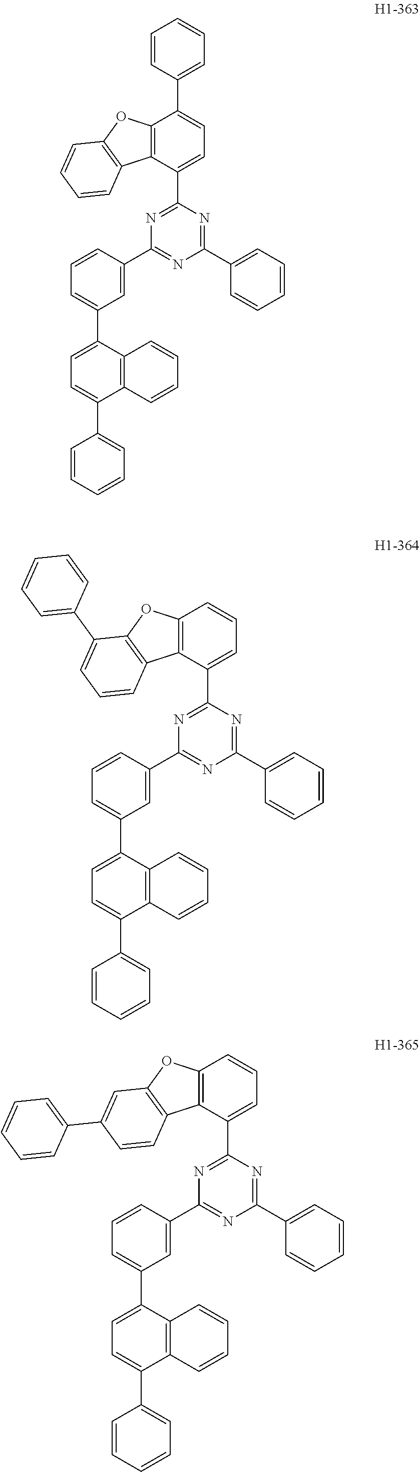 [SMILES: c1ccc(-c2nc(-c3cccc(-c4ccc(-c5ccccc5)c5ccccc45)c3)nc(-c3ccc(-c4ccccc4)c4oc5ccccc5c34)n2)cc1 ; c1ccc(-c2nc(-c3cccc(-c4ccc(-c5ccccc5)c5ccccc45)c3)nc(-c3cccc4oc5c(-c6ccccc6)cccc5c34)n2)cc1 ; c1ccc(-c2ccc3c(c2)oc2cccc(-c4nc(-c5ccccc5)nc(-c5cccc(-c6ccc(-c7ccccc7)c7ccccc67)c5)n4)c23)cc1]

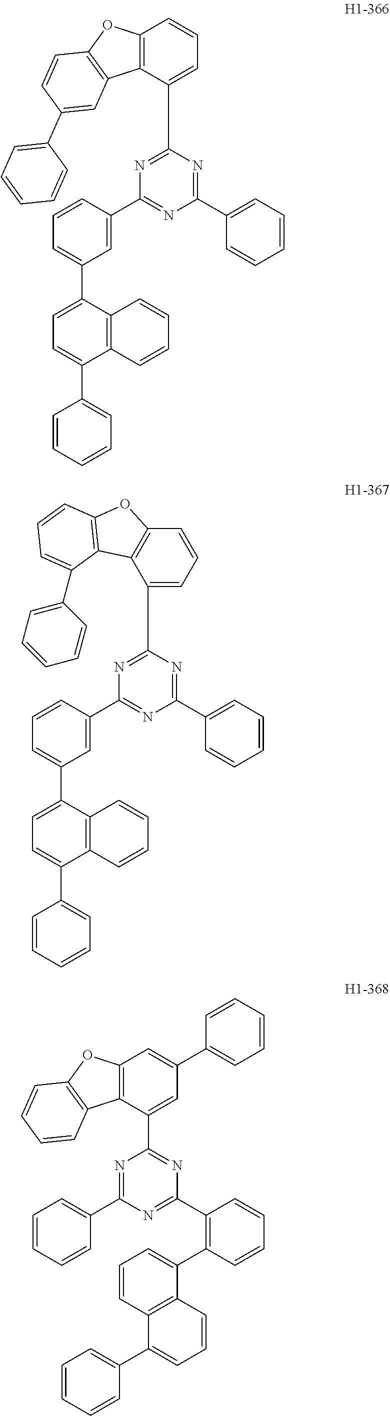

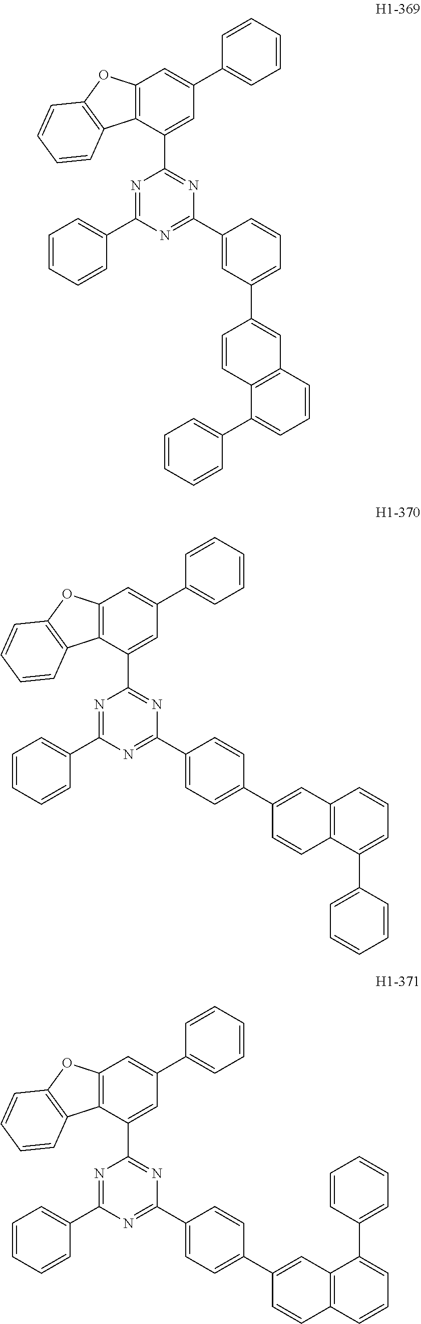 [SMILES: c1ccc(-c2cc(-c3nc(-c4ccccc4)nc(-c4cccc(-c5ccc6c(-c7ccccc7)cccc6c5)c4)n3)c3c(c2)oc2ccccc23)cc1 ; c1ccc(-c2cc(-c3nc(-c4ccccc4)nc(-c4ccc(-c5ccc6c(-c7ccccc7)cccc6c5)cc4)n3)c3c(c2)oc2ccccc23)cc1 ; c1ccc(-c2cc(-c3nc(-c4ccccc4)nc(-c4ccc(-c5ccc6cccc(-c7ccccc7)c6c5)cc4)n3)c3c(c2)oc2ccccc23)cc1]

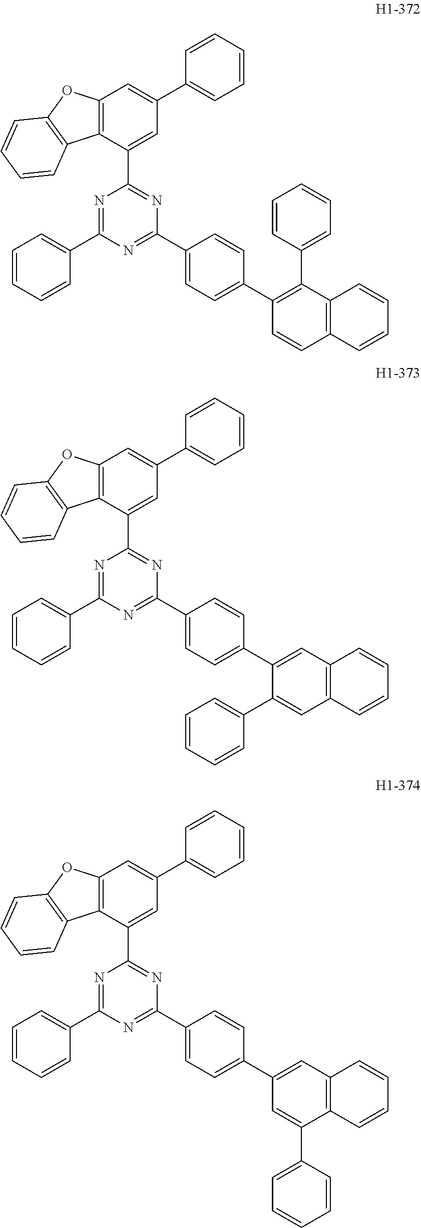

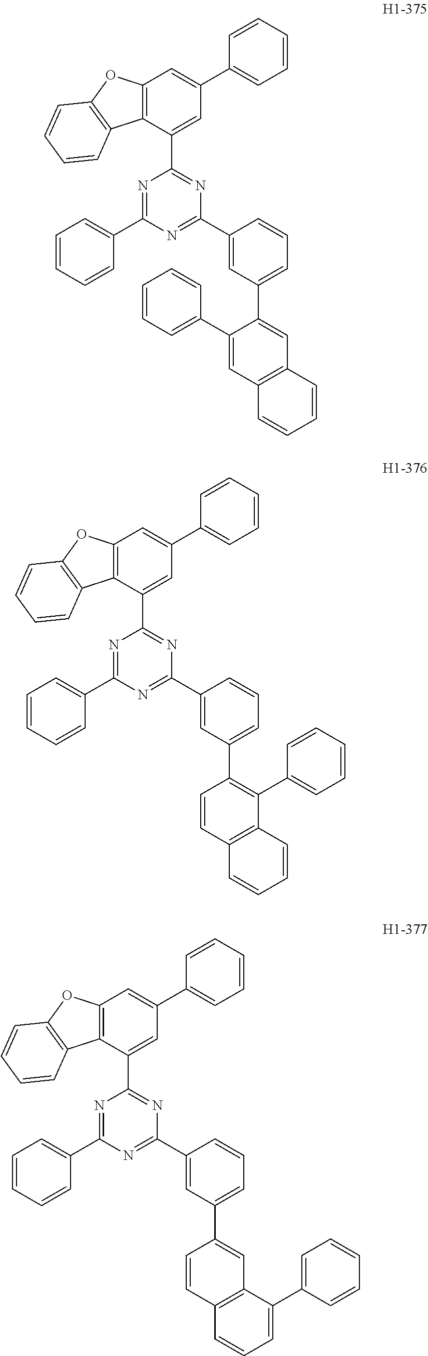 [SMILES: c1ccc(-c2cc(-c3nc(-c4ccccc4)nc(-c4cccc(-c5cc6ccccc6cc5-c5ccccc5)c4)n3)c3c(c2)oc2ccccc23)cc1 ; c1ccc(-c2cc(-c3nc(-c4ccccc4)nc(-c4cccc(-c5ccc6ccccc6c5-c5ccccc5)c4)n3)c3c(c2)oc2ccccc23)cc1 ; c1ccc(-c2cc(-c3nc(-c4ccccc4)nc(-c4cccc(-c5ccc6cccc(-c7ccccc7)c6c5)c4)n3)c3c(c2)oc2ccccc23)cc1]

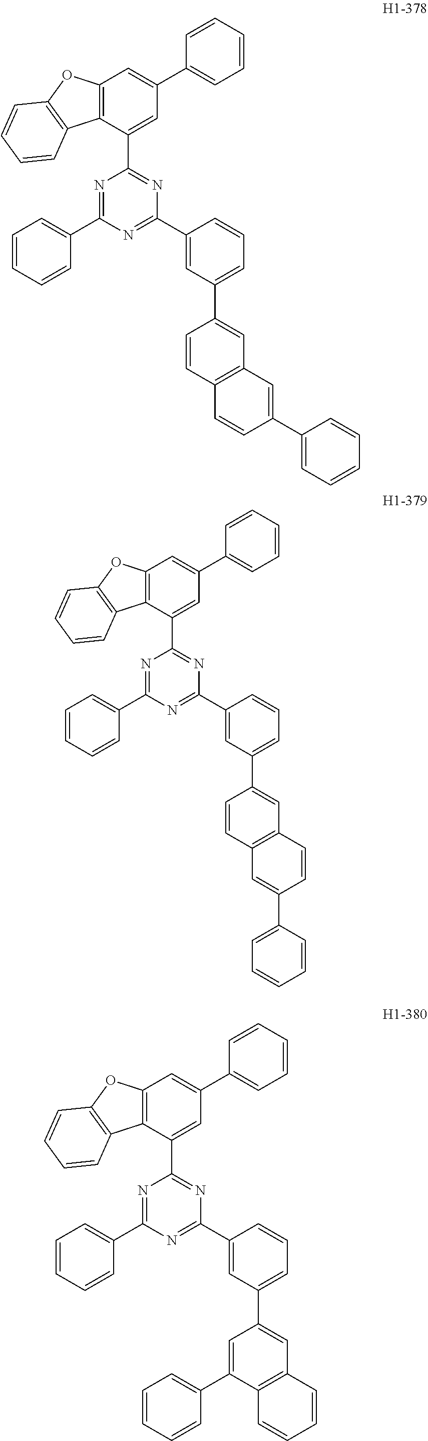

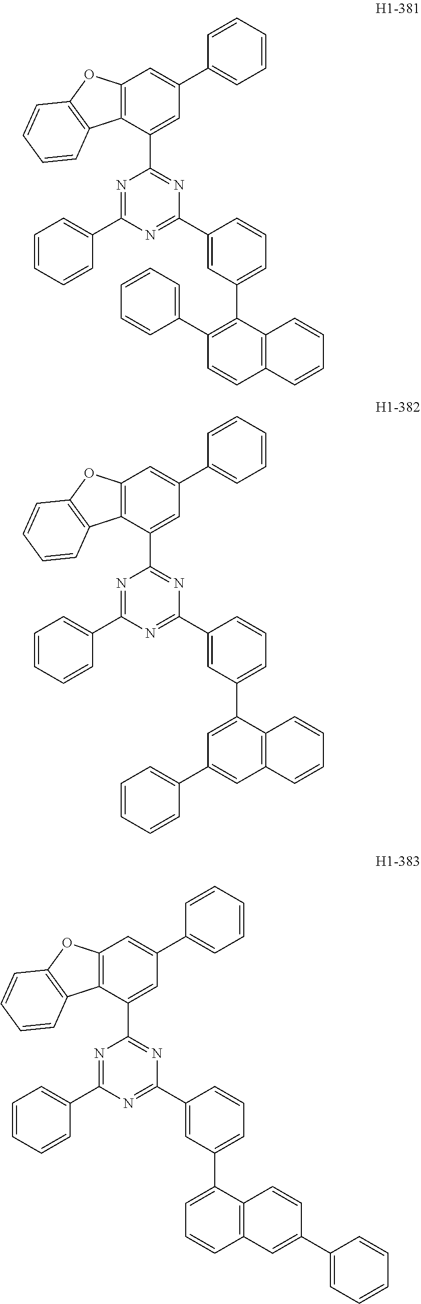

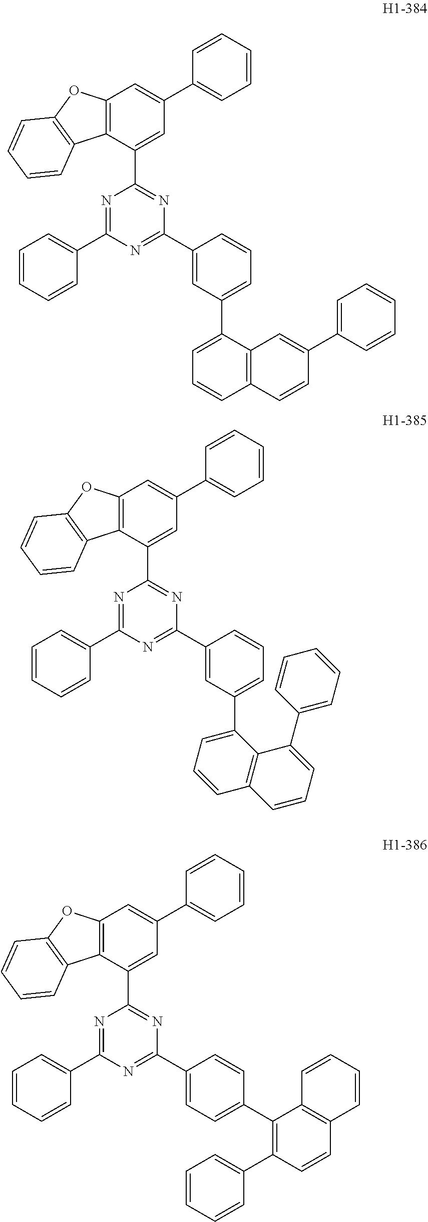

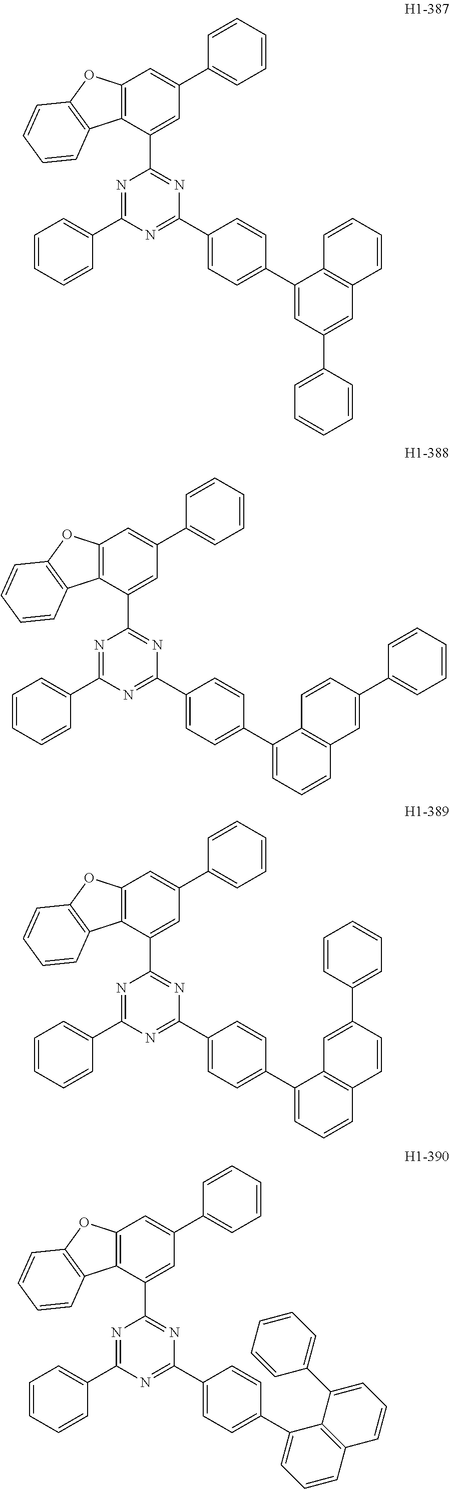 [SMILES: c1ccc(-c2cc(-c3ccc(-c4nc(-c5ccccc5)nc(-c5cc(-c6ccccc6)cc6oc7ccccc7c56)n4)cc3)c3ccccc3c2)cc1 ; c1ccc(-c2ccc3c(-c4ccc(-c5nc(-c6ccccc6)nc(-c6cc(-c7ccccc7)cc7oc8ccccc8c67)n5)cc4)cccc3c2)cc1 ; c1ccc(-c2cc(-c3nc(-c4ccccc4)nc(-c4ccc(-c5cccc6ccc(-c7ccccc7)cc56)cc4)n3)c3c(c2)oc2ccccc23)cc1 ; c1ccc(-c2cc(-c3nc(-c4ccccc4)nc(-c4ccc(-c5cccc6cccc(-c7ccccc7)c56)cc4)n3)c3c(c2)oc2ccccc23)cc1]

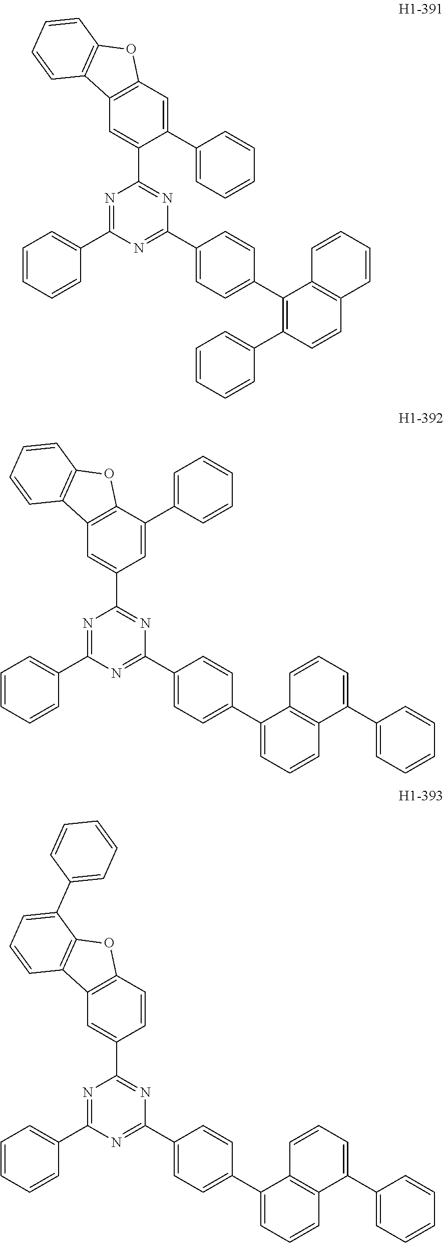 [SMILES: c1ccc(-c2nc(-c3ccc(-c4c(-c5ccccc5)ccc5ccccc45)cc3)nc(-c3cc4c(cc3-c3ccccc3)oc3ccccc34)n2)cc1 ; c1ccc(-c2nc(-c3ccc(-c4cccc5c(-c6ccccc6)cccc45)cc3)nc(-c3cc(-c4ccccc4)c4oc5ccccc5c4c3)n2)cc1 ; c1ccc(-c2nc(-c3ccc(-c4cccc5c(-c6ccccc6)cccc45)cc3)nc(-c3ccc4oc5c(-c6ccccc6)cccc5c4c3)n2)cc1]

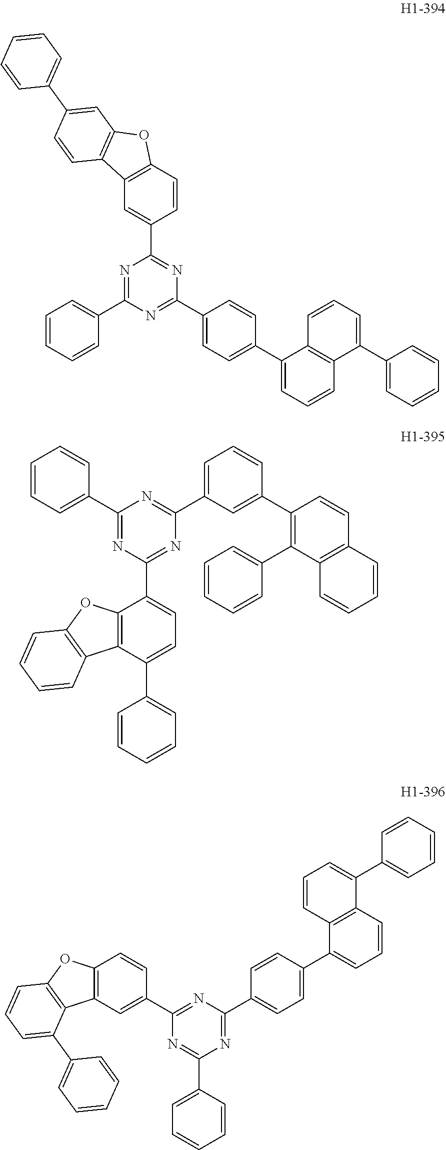 [SMILES: c1ccc(-c2ccc3c(c2)oc2ccc(-c4nc(-c5ccccc5)nc(-c5ccc(-c6cccc7c(-c8ccccc8)cccc67)cc5)n4)cc23)cc1 ; c1ccc(-c2nc(-c3cccc(-c4ccc5ccccc5c4-c4ccccc4)c3)nc(-c3ccc(-c4ccccc4)c4c3oc3ccccc34)n2)cc1 ; c1ccc(-c2nc(-c3ccc(-c4cccc5c(-c6ccccc6)cccc45)cc3)nc(-c3ccc4oc5cccc(-c6ccccc6)c5c4c3)n2)cc1]

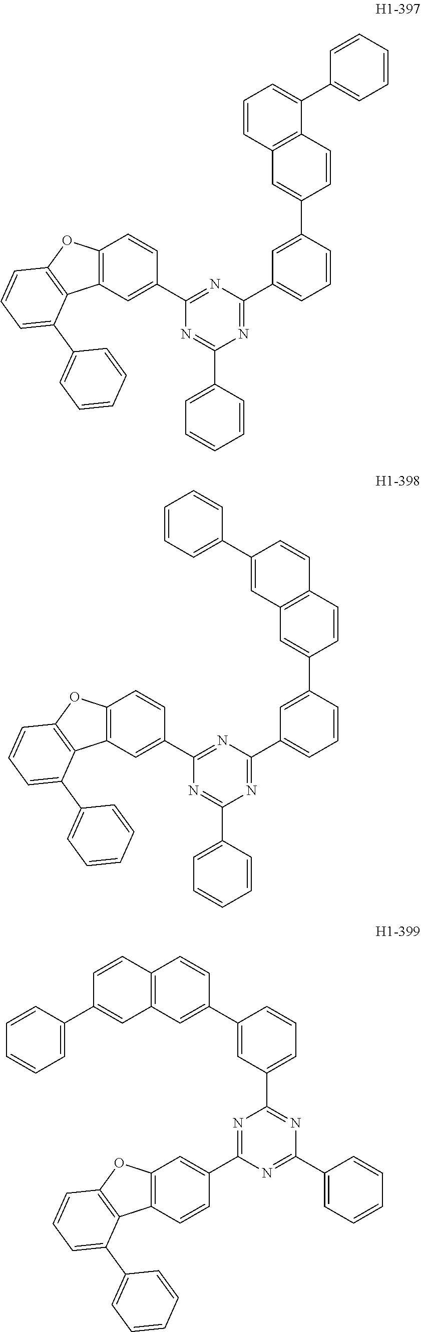

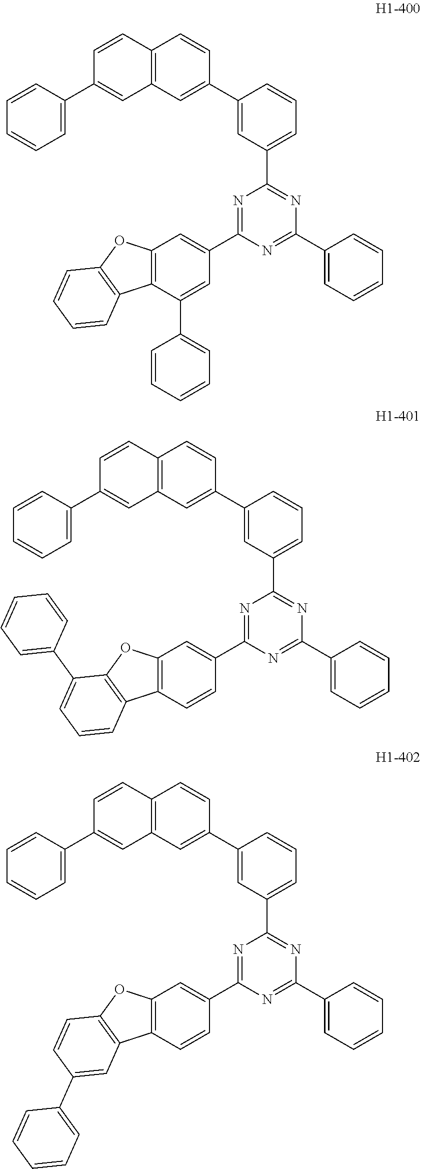

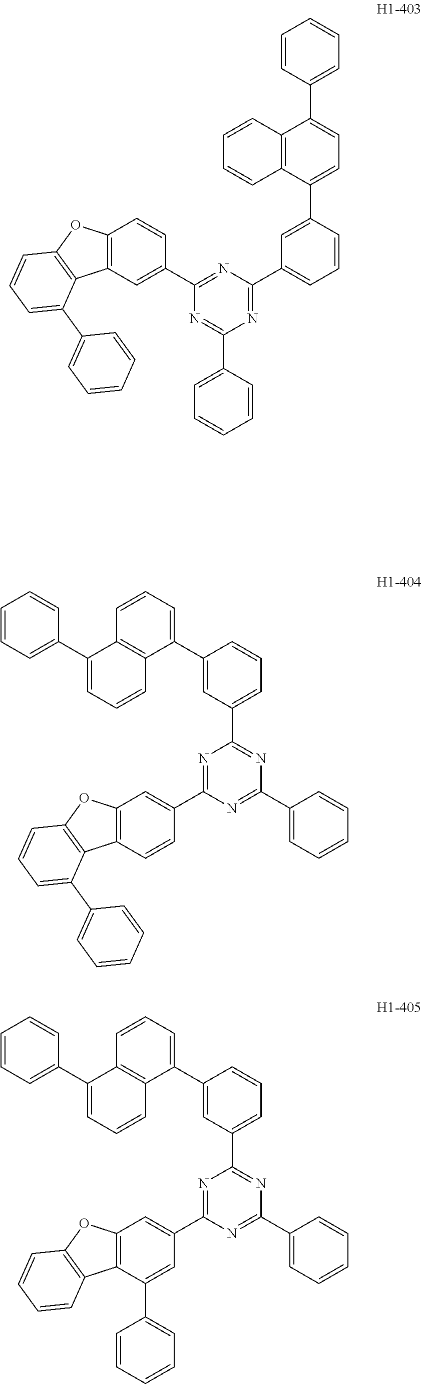 [SMILES: c1ccc(-c2nc(-c3cccc(-c4ccc(-c5ccccc5)c5ccccc45)c3)nc(-c3ccc4oc5cccc(-c6ccccc6)c5c4c3)n2)cc1 ; c1ccc(-c2nc(-c3cccc(-c4cccc5c(-c6ccccc6)cccc45)c3)nc(-c3ccc4c(c3)oc3cccc(-c5ccccc5)c34)n2)cc1 ; c1ccc(-c2nc(-c3cccc(-c4cccc5c(-c6ccccc6)cccc45)c3)nc(-c3cc(-c4ccccc4)c4c(c3)oc3ccccc34)n2)cc1]

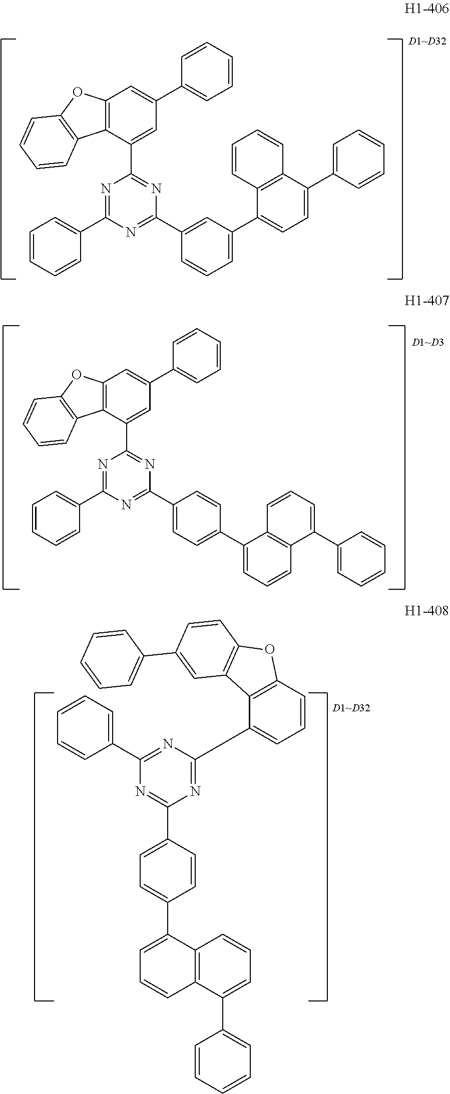

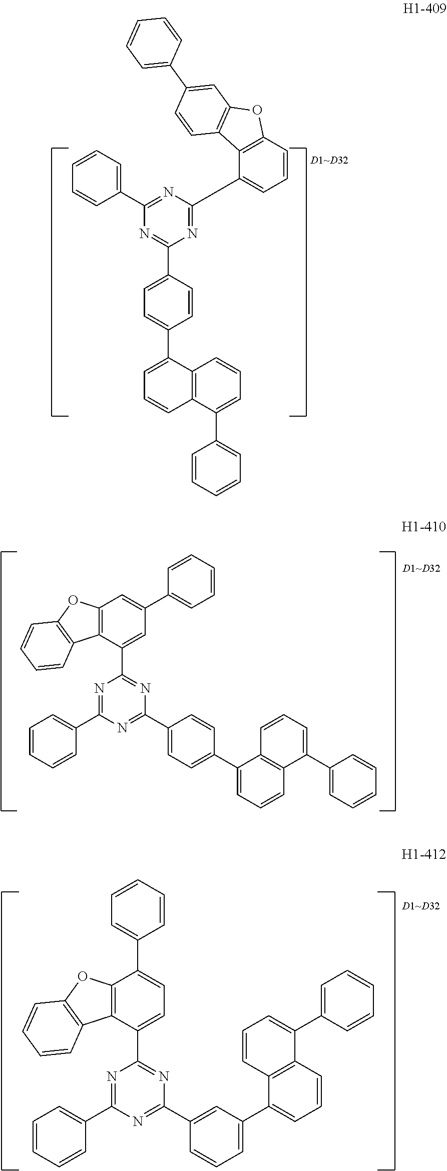

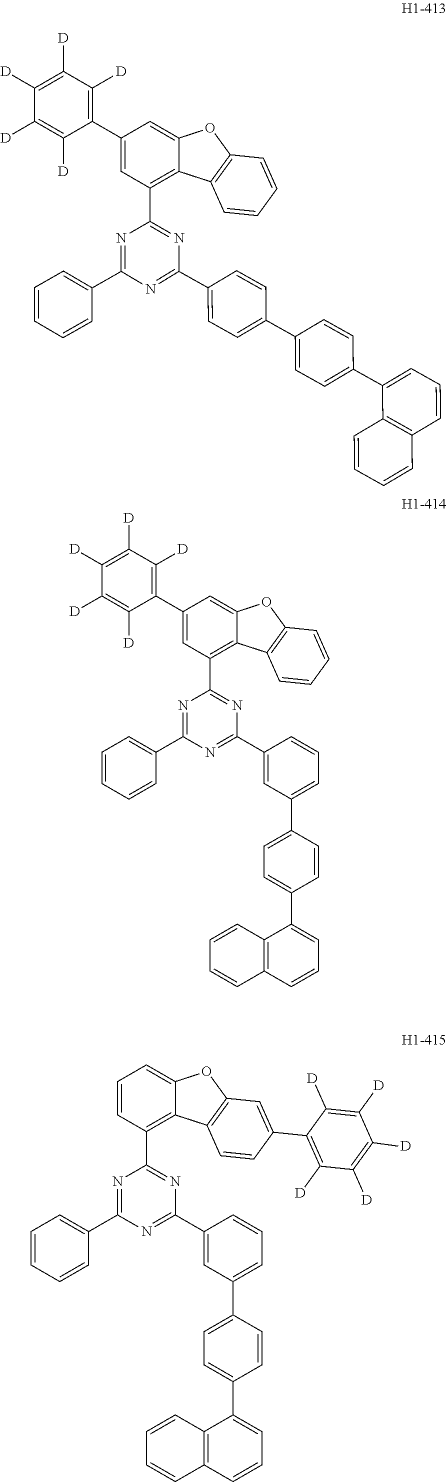

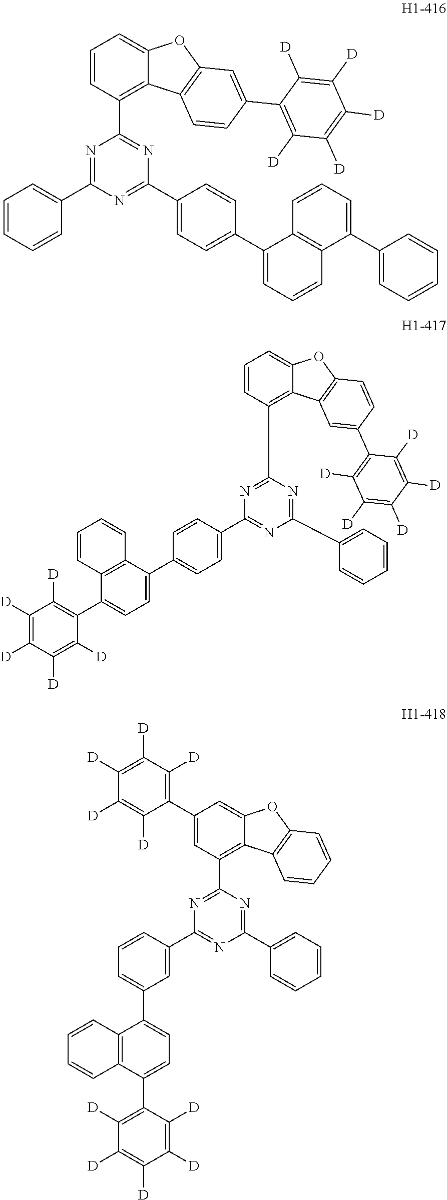 [SMILES: [2H]c1c([2H])c([2H])c(-c2ccc3c(c2)oc2cccc(-c4nc(-c5ccccc5)nc(-c5ccc(-c6cccc7c(-c8ccccc8)cccc67)cc5)n4)c23)c([2H])c1[2H] ; [2H]c1c([2H])c([2H])c(-c2ccc3oc4cccc(-c5nc(-c6ccccc6)nc(-c6ccc(-c7ccc(-c8c([2H])c([2H])c([2H])c([2H])c8[2H])c8ccccc78)cc6)n5)c4c3c2)c([2H])c1[2H] ; [2H]c1c([2H])c([2H])c(-c2cc(-c3nc(-c4ccccc4)nc(-c4cccc(-c5ccc(-c6c([2H])c([2H])c([2H])c([2H])c6[2H])c6ccccc56)c4)n3)c3c(c2)oc2ccccc23)c([2H])c1[2H]]

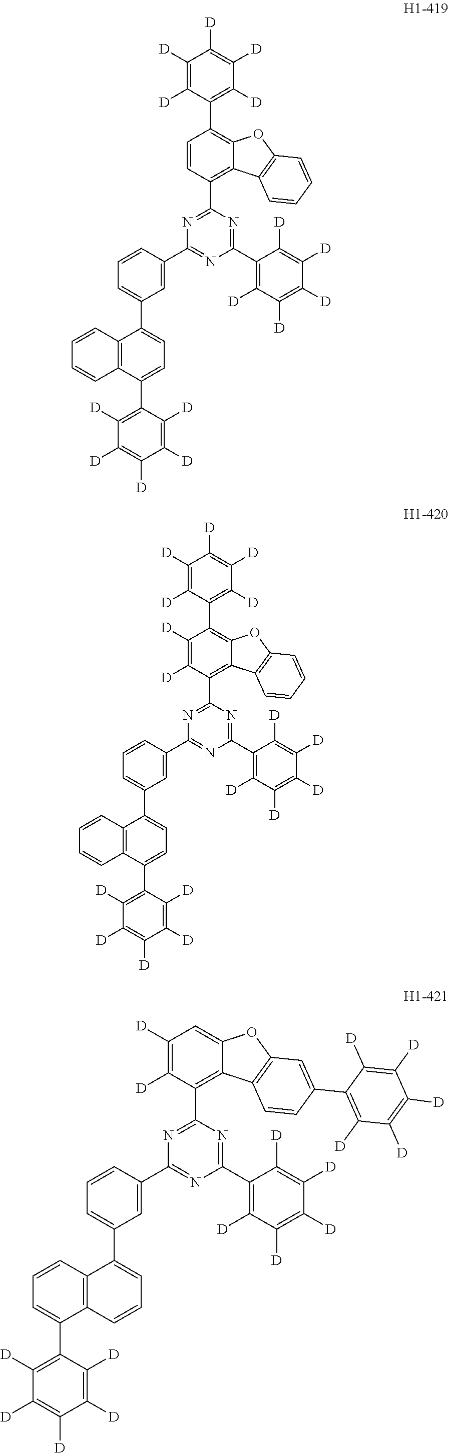

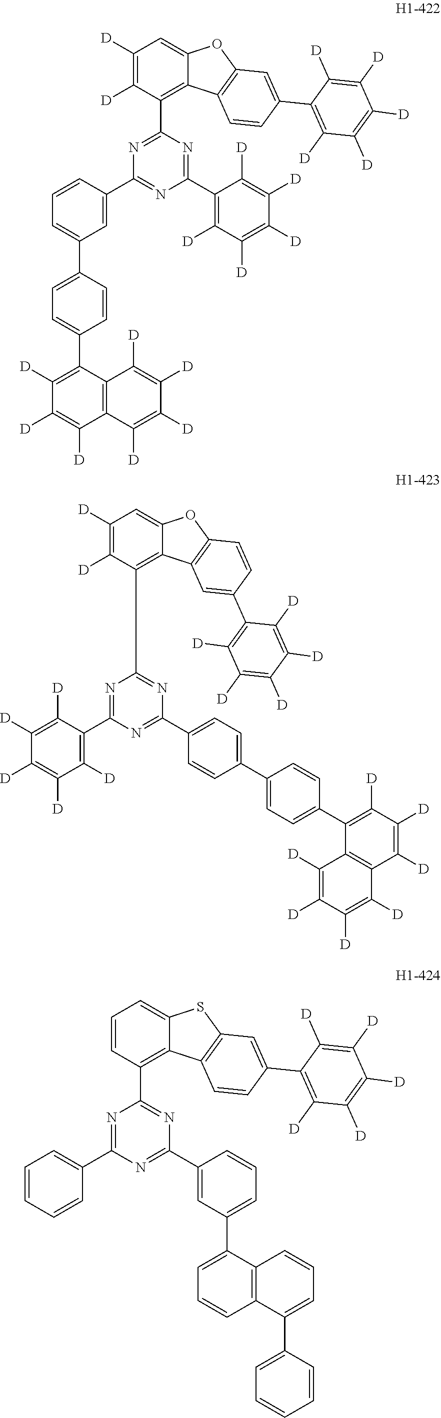

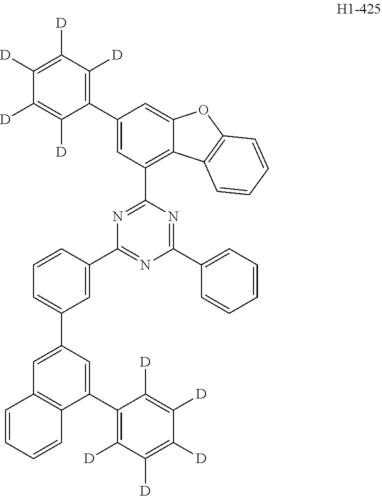 [SMILES: [2H]c1c([2H])c([2H])c(-c2cc(-c3nc(-c4ccccc4)nc(-c4cccc(-c5cc(-c6c([2H])c([2H])c([2H])c([2H])c6[2H])c6ccccc6c5)c4)n3)c3c(c2)oc2ccccc23)c([2H])c1[2H]]

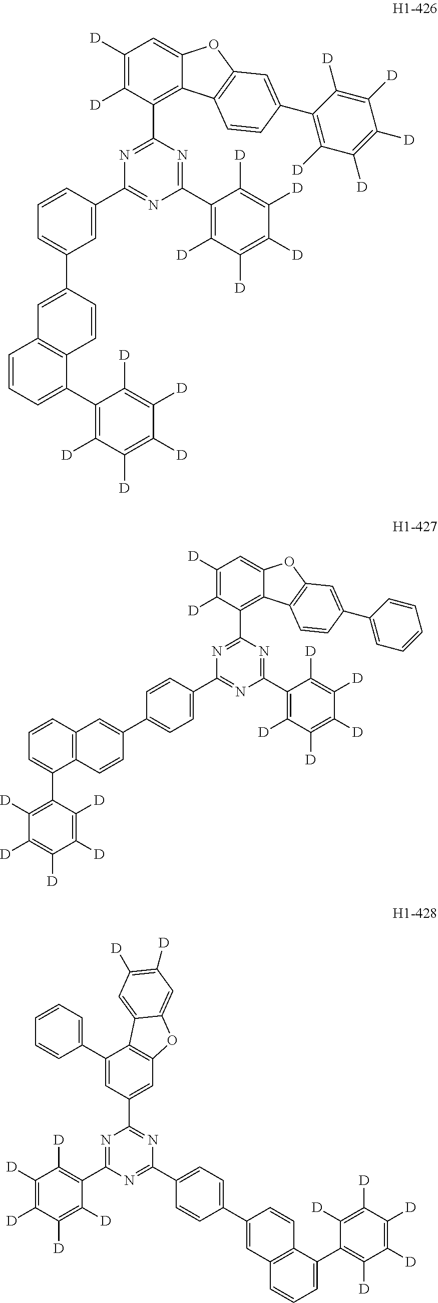 [SMILES: [2H]c1cc2oc3cc(-c4c([2H])c([2H])c([2H])c([2H])c4[2H])ccc3c2c(-c2nc(-c3cccc(-c4ccc5c(-c6c([2H])c([2H])c([2H])c([2H])c6[2H])cccc5c4)c3)nc(-c3c([2H])c([2H])c([2H])c([2H])c3[2H])n2)c1[2H] ; [2H]c1cc2oc3cc(-c4ccccc4)ccc3c2c(-c2nc(-c3ccc(-c4ccc5c(-c6c([2H])c([2H])c([2H])c([2H])c6[2H])cccc5c4)cc3)nc(-c3c([2H])c([2H])c([2H])c([2H])c3[2H])n2)c1[2H] ; [2H]c1cc2oc3cc(-c4nc(-c5ccc(-c6ccc7c(-c8c([2H])c([2H])c([2H])c([2H])c8[2H])cccc7c6)cc5)nc(-c5c([2H])c([2H])c([2H])c([2H])c5[2H])n4)cc(-c4ccccc4)c3c2cc1[2H]]

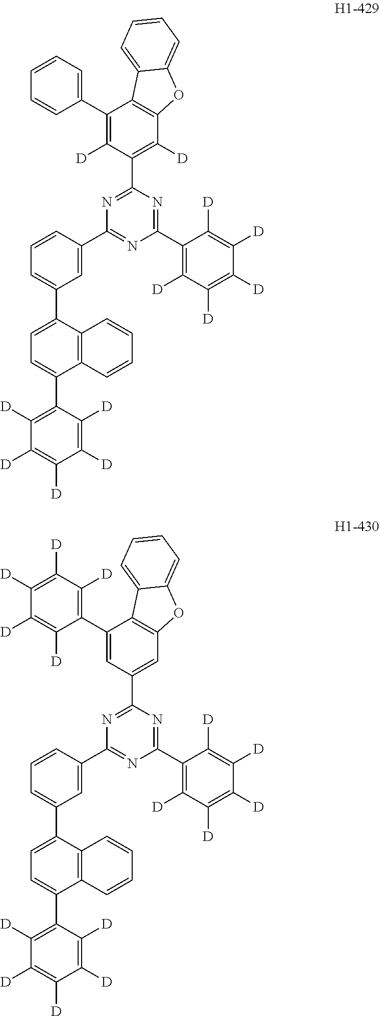 [SMILES: [2H]c1c([2H])c([2H])c(-c2nc(-c3cccc(-c4ccc(-c5c([2H])c([2H])c([2H])c([2H])c5[2H])c5ccccc45)c3)nc(-c3c([2H])c(-c4ccccc4)c4c(oc5ccccc54)c3[2H])n2)c([2H])c1[2H] ; [2H]c1c([2H])c([2H])c(-c2nc(-c3cccc(-c4ccc(-c5c([2H])c([2H])c([2H])c([2H])c5[2H])c5ccccc45)c3)nc(-c3cc(-c4c([2H])c([2H])c([2H])c([2H])c4[2H])c4c(c3)oc3ccccc34)n2)c([2H])c1[2H]]

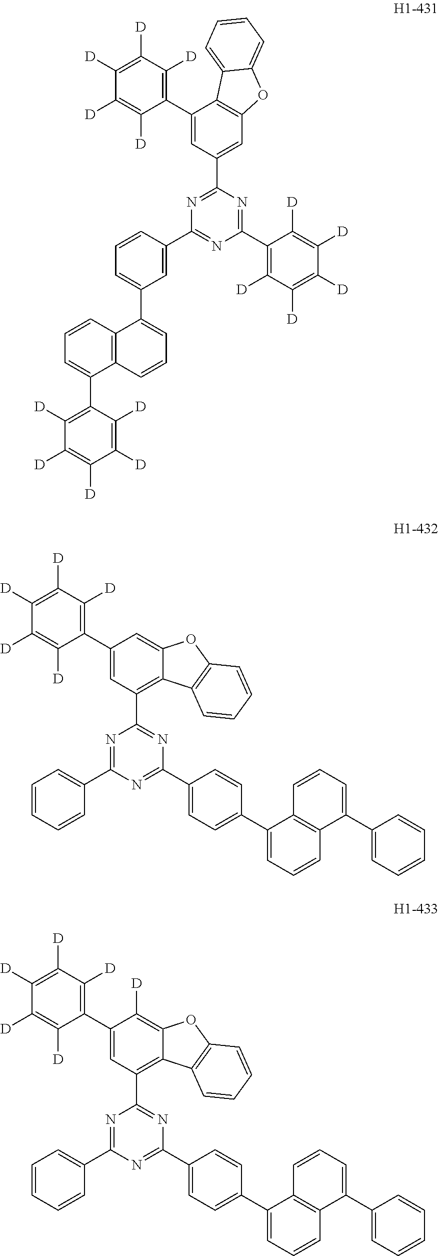 [SMILES: [2H]c1c([2H])c([2H])c(-c2nc(-c3cccc(-c4cccc5c(-c6c([2H])c([2H])c([2H])c([2H])c6[2H])cccc45)c3)nc(-c3cc(-c4c([2H])c([2H])c([2H])c([2H])c4[2H])c4c(c3)oc3ccccc34)n2)c([2H])c1[2H] ; [2H]c1c([2H])c([2H])c(-c2cc(-c3nc(-c4ccccc4)nc(-c4ccc(-c5cccc6c(-c7ccccc7)cccc56)cc4)n3)c3c(c2)oc2ccccc23)c([2H])c1[2H] ; [2H]c1c([2H])c([2H])c(-c2cc(-c3nc(-c4ccccc4)nc(-c4ccc(-c5cccc6c(-c7ccccc7)cccc56)cc4)n3)c3c(oc4ccccc43)c2[2H])c([2H])c1[2H]]

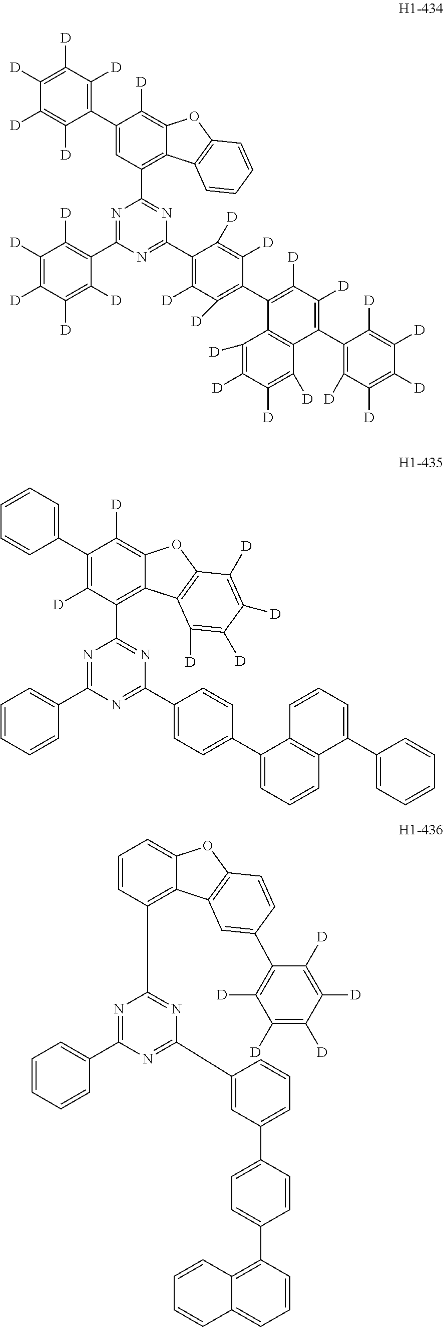 [SMILES: [2H]c1c([2H])c([2H])c(-c2nc(-c3c([2H])c([2H])c(-c4c([2H])c([2H])c(-c5c([2H])c([2H])c([2H])c([2H])c5[2H])c5c([2H])c([2H])c([2H])c([2H])c45)c([2H])c3[2H])nc(-c3cc(-c4c([2H])c([2H])c([2H])c([2H])c4[2H])c([2H])c4oc5ccccc5c34)n2)c([2H])c1[2H] ; [2H]c1c([2H])c([2H])c2c(oc3c([2H])c(-c4ccccc4)c([2H])c(-c4nc(-c5ccccc5)nc(-c5ccc(-c6cccc7c(-c8ccccc8)cccc67)cc5)n4)c32)c1[2H] ; [2H]c1c([2H])c([2H])c(-c2ccc3oc4cccc(-c5nc(-c6ccccc6)nc(-c6cccc(-c7ccc(-c8cccc9ccccc89)cc7)c6)n5)c4c3c2)c([2H])c1[2H]]

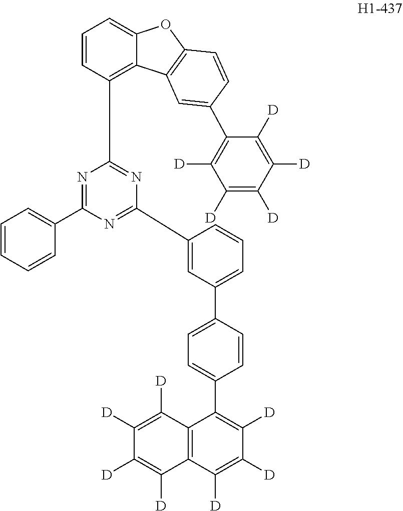 [SMILES: [2H]c1c([2H])c([2H])c(-c2ccc3oc4cccc(-c5nc(-c6ccccc6)nc(-c6cccc(-c7ccc(-c8c([2H])c([2H])c([2H])c9c([2H])c([2H])c([2H])c([2H])c89)cc7)c6)n5)c4c3c2)c([2H])c1[2H]]

The compound represented by Formula 1 according to the present disclosure can be prepared as shown in the following Reaction Scheme 1, but is not limited thereto.

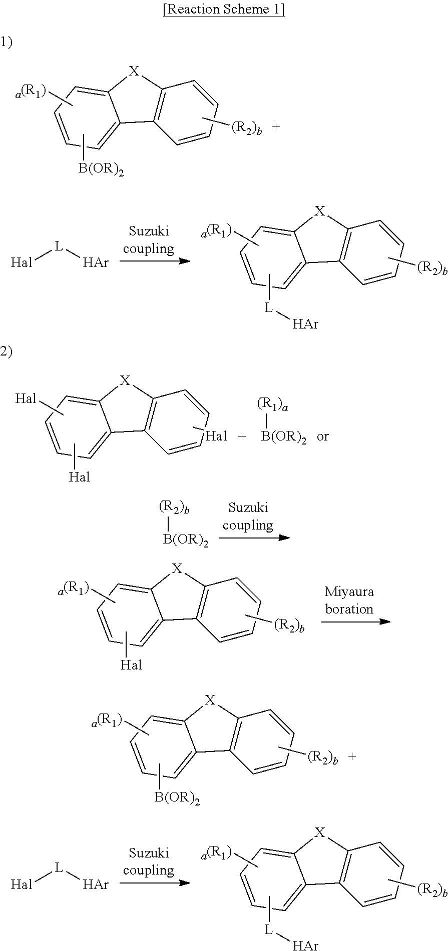

In Reaction Scheme 1, X, R₁, R₂, L, HAr, a, and b are as defined in Formula 1, R represents hydrogen or (C₁-C₃₀)alkyl, and Hal means a halogen.

The second host material, which is another host material according to one embodiment, may include a compound represented by the following Formula 2.

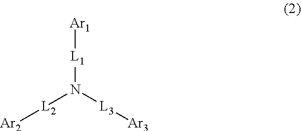

In Formula 2,
L₁ to L₃ each independently represent a single bond, a substituted or unsubstituted (C₆-C₃₀)arylene, or a substituted or unsubstituted (3- to 30-membered)heteroarylene;
Ar₁ represents a substituted or unsubstituted (C₆-C₃₀)aryl, or a substituted or unsubstituted (3- to 30-membered)heteroaryl; and
Ar₂ and Ar₃ each independently represent hydrogen, deuterium, a halogen, a cyano, a substituted or unsubstituted (C₁-C₃₀)alkyl, a substituted or unsubstituted (C₆-C₃₀)aryl, a substituted or unsubstituted (3- to 30-membered)heteroaryl, a substituted or unsubstituted (C₃-C₃₀)cycloalkyl, a substituted or unsubstituted (C₁-C₃₀)alkoxy, a substituted or unsubstituted tri(C₁-C₃₀)alkylsilyl, a substituted or unsubstituted di(C₁-C₃₀)alkyl(C₆-C₃₀)arylsilyl, a substituted or unsubstituted (C₁-C₃₀)alkyldi(C₆-C₃₀)arylsilyl, a substituted or unsubstituted tri(C₆-C₃₀)arylsilyl, a substituted or unsubstituted mono- or di(C₁-C₃₀)alkylamino, a substituted or unsubstituted mono- or di(C₂-C₃₀)alkenylamino, a substituted or unsubstituted (C₁-C₃₀)alkyl(C₂-C₃₀)alkenylamino, a substituted or unsubstituted mono- or di(C₆-C₃₀)arylamino, a substituted or unsubstituted (C₁-C₃₀)alkyl(C₆-C₃₀)arylamino, a substituted or unsubstituted mono- or di(3- to 30-membered)heteroarylamino, a substituted or unsubstituted (C₁-C₃₀)alkyl(3- to 30-membered)heteroarylamino, a substituted or unsubstituted (C₂-C₃₀)alkenyl(C₆-C₃₀)arylamino, a substituted or unsubstituted (C₂-C₃₀)alkenyl(3- to 30-membered)heteroarylamino, a substituted or unsubstituted (C₆-C₃₀)aryl(3- to 30-membered)heteroarylamino, or a substituted or unsubstituted fused ring of (C₃-C₃₀) aliphatic ring and (C₆-C₃₀) aromatic ring; or may be linked to the adjacent substituents to form a ring(s).

In one embodiment, L₁ to L₃ each independently may be a single bond, a substituted or unsubstituted (C₆-C₃₀)arylene, or a substituted or unsubstituted (3- to 30-membered)heteroarylene, preferably L₁ to L₃ each independently may be a single bond, a substituted or unsubstituted (C₆-C₂₅)arylene, or a substituted or unsubstituted (3- to 25-membered)heteroarylene. For example, L₁ to L₃ each independently may be a single bond, a substituted or unsubstituted phenylene, a substituted or unsubstituted biphenylene, a substituted or unsubstituted naphthylene, a substituted or unsubstituted phenanthrenylene, a substituted or unsubstituted pyridinylene, a substituted or unsubstituted dibenzofuranylene, a substituted or unsubstituted dibenzothiophenylene, or a substituted or unsubstituted carbazolylene, which may be substituted with one or more selected from the group consisting of deuterium, phenyl, and naphthyl.

In one embodiment, Ar₁ may be a substituted or unsubstituted (C₆-C₃₀)aryl, or a substituted or unsubstituted (3- to 30-membered)heteroaryl, preferably Ar₁ may be a substituted or unsubstituted (C₆-C₂₅)aryl, or a substituted or unsubstituted (3- to 25-membered)heteroaryl. For example, Ar₁ may be a substituted or unsubstituted phenanthrenyl, a substituted or unsubstituted chrysenyl, a substituted or unsubstituted benzophenanthrenyl, a substituted or unsubstituted phenanthrooxazolyl, a substituted or unsubstituted phenanthrothiazolyl, a substituted or unsubstituted benzonaphthofuranyl, or a substituted or unsubstituted benzonaphthothiophenyl.

In one embodiment, Ar₂ and Ar₃ each independently may be hydrogen, deuterium, a halogen, a cyano, a substituted or unsubstituted (C₆-C₃₀)aryl, a substituted or unsubstituted (3- to 30-membered)heteroaryl, a substituted or unsubstituted (C₃-C₃₀)cycloalkyl, tri(C₆-C₃₀)arylsilyl, a substituted or unsubstituted mono- or di(C₆-C₃₀)arylamino, a substituted or unsubstituted mono- or di(3- to 30-membered)heteroarylamino, a substituted or unsubstituted (C₆-C₃₀)aryl(3- to 30-membered)heteroarylamino, or a substituted or unsubstituted fused ring of (C₃-C₃₀) aliphatic ring and (C₆-C₃₀) aromatic ring. For example, Ar₂ and Ar₃ each independently may be a substituted or unsubstituted phenyl, a substituted or unsubstituted biphenyl, a substituted or unsubstituted o-terphenyl, a substituted or unsubstituted m-terphenyl, a substituted or unsubstituted p-terphenyl, a substituted or unsubstituted naphthyl, a substituted or unsubstituted quaterphenyl, a substituted or unsubstituted phenanthrenyl, a substituted or unsubstituted anthracenyl, a substituted or unsubstituted chrysenyl, a substituted or unsubstituted benzonaphthalenyl, a substituted or unsubstituted fluorenyl, a substituted or unsubstituted benzofluorenyl, a substituted or unsubstituted spirobifluorenyl, a substituted or unsubstituted fluoranthenyl, a substituted or unsubstituted dibenzonaphthocycloheptanyl, a substituted or unsubstituted pyridinyl, a substituted or unsubstituted benzofuranyl, a substituted or unsubstituted dibenzofuranyl, a substituted or unsubstituted benzonaphthofuranyl, a substituted or unsubstituted dibenzothiophenyl, a substituted or unsubstituted benzonaphthothiophenyl, a substituted or unsubstituted carbazolyl, a substituted or unsubstituted benzofuropyridinyl, a substituted or unsubstituted dibenzoselenophenyl, a substituted or unsubstituted benzonaphthoselenophenyl, a substituted or unsubstituted benzoimidazolyl, a substituted or unsubstituted phenoxazinyl, a substituted or unsubstituted triphenylsilyl, or amino unsubstituted or substituted with 1 or 2 substituents, which may be substituted with one or more selected from the group consisting of deuterium, phenyl, naphthyl, and biphenyl, and wherein the substituted amino each independently may be substituted with one or more selected from the group consisting of phenyl, biphenyl, naphthyl, pyridinyl, dibenzofuranyl, and dibenzothiophenyl.

According to one embodiment, the compound represented by Formula 2 may be represented by any one of the following Formulas 2-1 to 2-3.

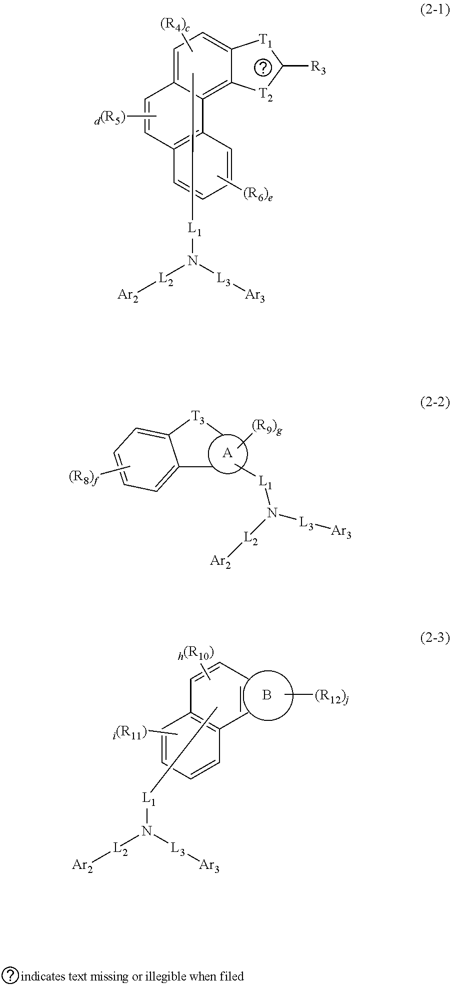

In Formulas 2-1 to 2-3,
T₁ and T₂ each independently represent —N═, —NR₇—, —O—, or —S—, provided that any one of T₁ and T₂ is —N═, and the other of T₁ and T₂ is —NR₇—, —O—, or —S—;
T₃ represents O or S;
ring A represents a substituted or unsubstituted phenanthrene;
ring B represents a substituted or unsubstituted benzene, or a substituted or unsubstituted naphthalene;
R₃ represents a substituted or unsubstituted (C₆-C₃₀)aryl, or a substituted or unsubstituted (3- to 30-membered)heteroaryl;
R₄ to R₁₂ each independently represent hydrogen, deuterium, a halogen, a cyano, a substituted or unsubstituted (C₁-C₃₀)alkyl, a substituted or unsubstituted (C₆-C₃₀)aryl, a substituted or unsubstituted (3- to 30-membered)heteroaryl, a substituted or unsubstituted (C₃-C₃₀)cycloalkyl, a substituted or unsubstituted (C₁-C₃₀)alkoxy, a substituted or unsubstituted tri(C₁-C₃₀)alkylsilyl, a substituted or unsubstituted di(C₁-C₃₀)alkyl(C₆-C₃₀)arylsilyl, a substituted or unsubstituted (C₁-C₃₀)alkyldi(C₆-C₃₀)arylsilyl, a substituted or unsubstituted tri(C₆-C₃₀)arylsilyl, a substituted or unsubstituted fused ring of (C₃-C₃₀) aliphatic ring and (C₆-C₃₀) aromatic ring, a substituted or unsubstituted mono- or di(C₁-C₃₀)alkylamino, a substituted or unsubstituted mono- or di(C₂-C₃₀)alkenylamino, a substituted or unsubstituted (C₁-C₃₀)alkyl(C₂-C₃₀)alkenylamino, a substituted or unsubstituted (C₁-C₃₀)alkyl(C₆-C₃₀)arylamino, a substituted or unsubstituted (C₁-C₃₀)alkyl(3- to 30-membered)heteroarylamino, a substituted or unsubstituted (C₂-C₃₀)alkenyl(C₆-C₃₀)arylamino, a substituted or unsubstituted (C₂-C₃₀)alkenyl(3- to 30-membered)heteroarylamino, a substituted or unsubstituted mono- or di(C₆-C₃₀)arylamino, a substituted or unsubstituted mono- or di(3- to 30-membered)heteroarylamino, or a substituted or unsubstituted (C₆-C₃₀)aryl(3- to 30-membered)heteroarylamino; or may be linked to the adjacent substituents to form a ring(s);
c, d, and h each independently represent an integer of 1 or 2, e, f, and i each independently represent an integer of 1 to 4, g is an integer of 1 to 7, j is an integer of 1 to 6, and when c to j are an integer of 2 or more, each of R₄ to each of R₆ and each of R₈ to each of R₁₂ may be the same as or different from each other; and
L₁ to L₃, Ar₂, and Ar₃ are as defined in Formula 2.

According to one embodiment, when L₃ in Formula 2-1 is a substituted or unsubstituted phenylene, Formula 2-1 may be represented by the following Formula 2-1-a.

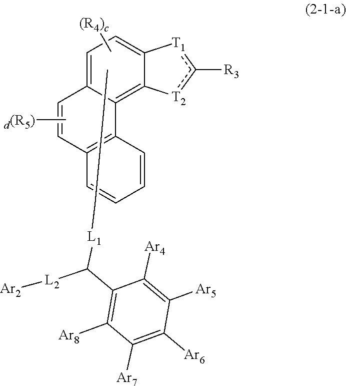

In Formula 2-1-a,
Ar₄ to Ar₈ are each as defined for Ar₃ in Formula 2, provided that Ar₆ is not a substituted or unsubstituted heteroaryl; and
T₁, T₂, R₃ to R₆, and c to e are each as defined in Formula 2-1, and L₁, L₂, and Ar₂ are each as defined in Formula 2.

According to one embodiment, the compound represented by Formula 2-2 may be represented by any one of the following Formulas 2-2a to 2-2c.

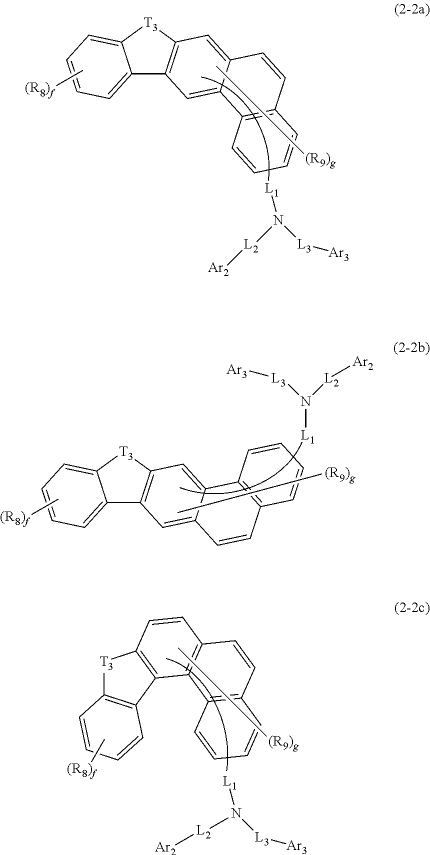

In Formulas 2-2a to 2-2c,
T₃, R₈, R₉, L₁ to L₃, Ar₂, Ar₃, f, and g are as defined in Formula 2-2.

According to one embodiment, the compound represented by Formula 2-3 may be represented by any one of the following Formulas 2-3a to 2-3d.

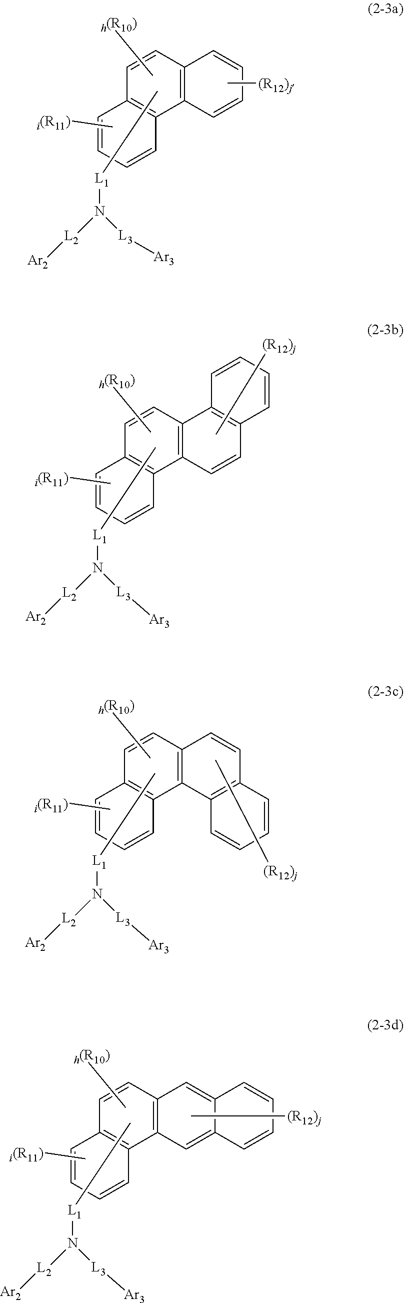

In Formulas 2-3a to 2-3d,
j′ is an integer of 1 to 4; and
when j′ is an integer of 2 or more, each of R₁₂ may be the same as or different from each other; and
R₁₀ to R₁₂, L₁ to L₃, Ar₂, Ar₃, and h to j are as defined in Formula 2-3.

According to one embodiment, the second host material may be more specifically illustrated by the following compounds, but is not limited thereto:

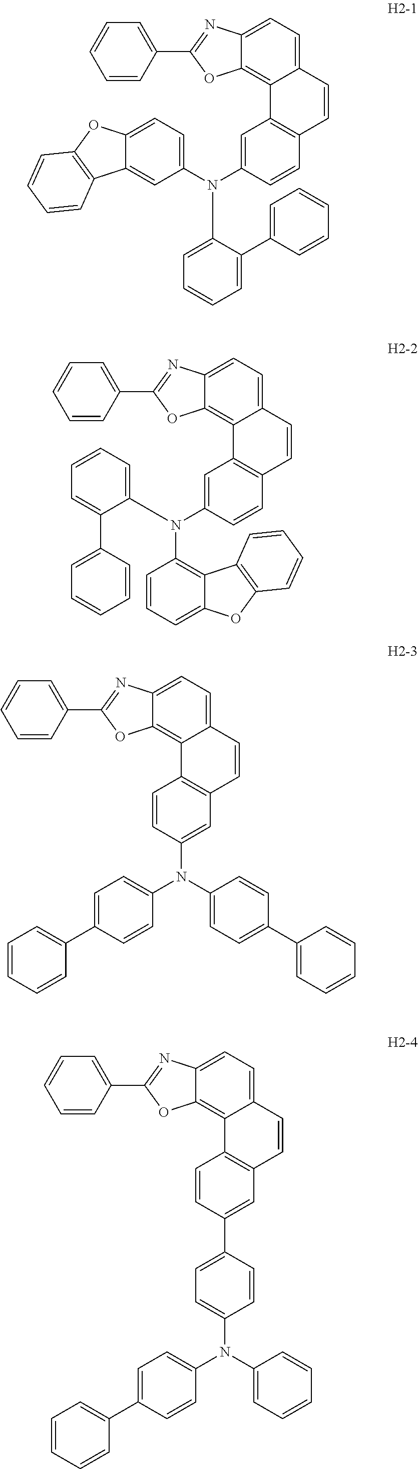 [SMILES: c1ccc(-c2nc3ccc4ccc5ccc(N(c6ccc7oc8ccccc8c7c6)c6ccccc6-c6ccccc6)cc5c4c3o2)cc1 ; c1ccc(-c2nc3ccc4ccc5ccc(N(c6ccccc6-c6ccccc6)c6cccc7oc8ccccc8c67)cc5c4c3o2)cc1 ; c1ccc(-c2ccc(N(c3ccc(-c4ccccc4)cc3)c3ccc4c(ccc5ccc6nc(-c7ccccc7)oc6c54)c3)cc2)cc1 ; c1ccc(-c2ccc(N(c3ccccc3)c3ccc(-c4ccc5c(ccc6ccc7nc(-c8ccccc8)oc7c65)c4)cc3)cc2)cc1]

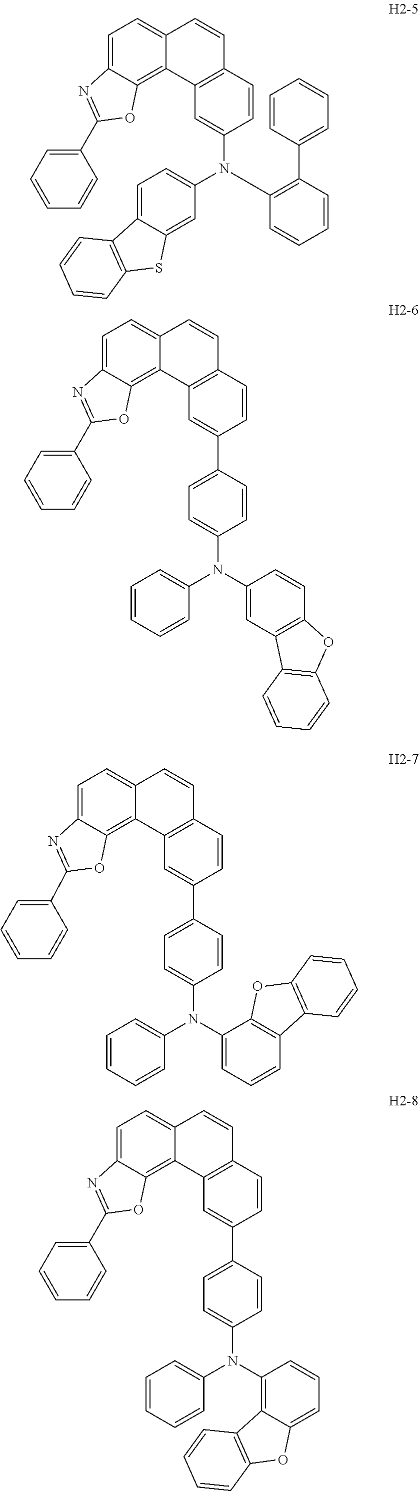

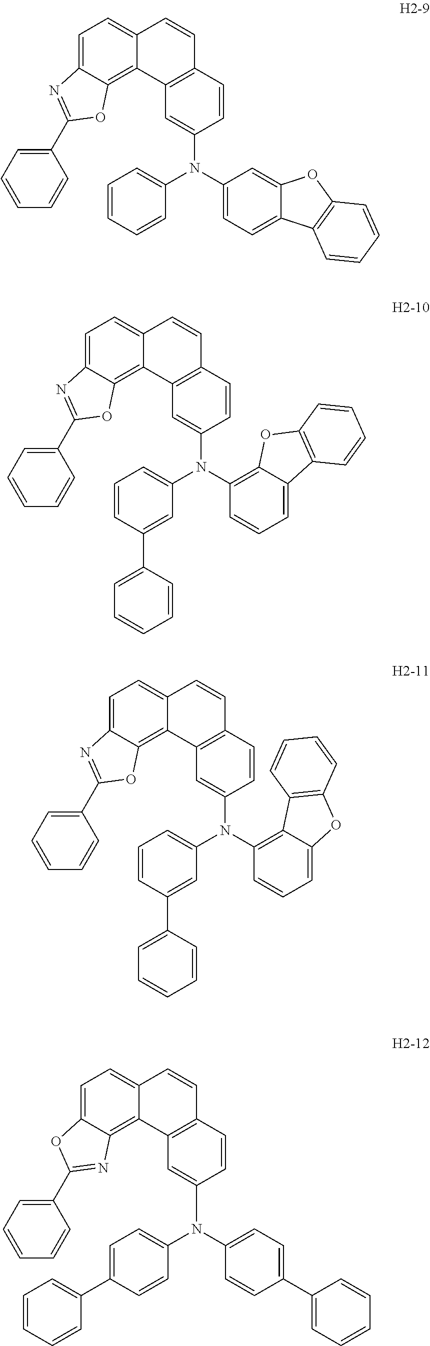 [SMILES: c1ccc(-c2nc3ccc4ccc5ccc(N(c6ccccc6)c6ccc7c(c6)oc6ccccc67)cc5c4c3o2)cc1 ; c1ccc(-c2cccc(N(c3ccc4ccc5ccc6nc(-c7ccccc7)oc6c5c4c3)c3cccc4c3oc3ccccc34)c2)cc1 ; c1ccc(-c2cccc(N(c3ccc4ccc5ccc6nc(-c7ccccc7)oc6c5c4c3)c3cccc4oc5ccccc5c34)c2)cc1 ; c1ccc(-c2ccc(N(c3ccc(-c4ccccc4)cc3)c3ccc4ccc5ccc6oc(-c7ccccc7)nc6c5c4c3)cc2)cc1]

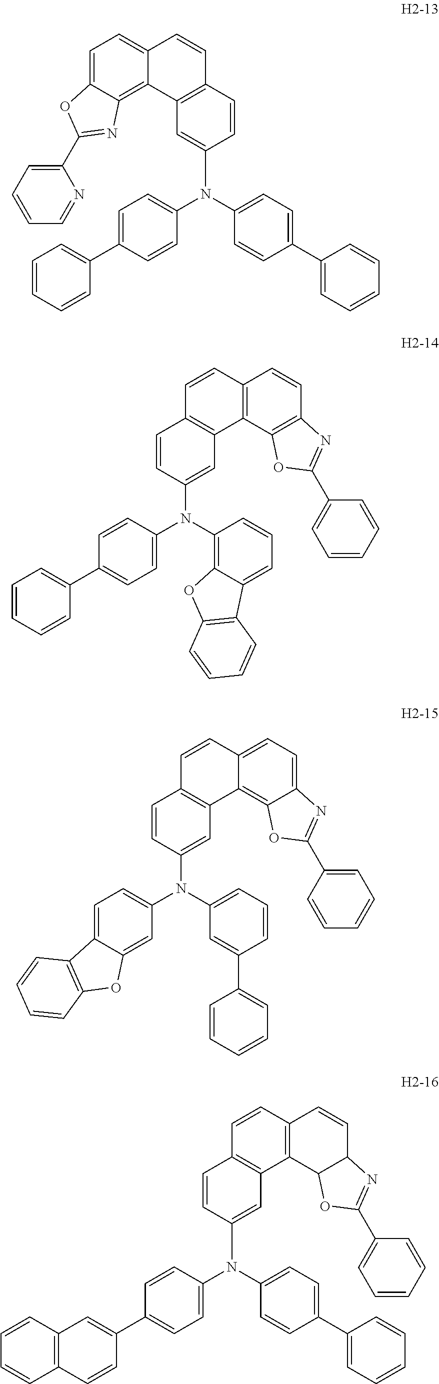 [SMILES: c1ccc(-c2ccc(N(c3ccc(-c4ccccc4)cc3)c3ccc4ccc5ccc6oc(-c7ccccn7)nc6c5c4c3)cc2)cc1 ; c1ccc(-c2ccc(N(c3ccc4ccc5ccc6nc(-c7ccccc7)oc6c5c4c3)c3cccc4c3oc3ccccc34)cc2)cc1 ; c1ccc(-c2cccc(N(c3ccc4c(c3)oc3ccccc34)c3ccc4ccc5ccc6nc(-c7ccccc7)oc6c5c4c3)c2)cc1 ; C1=CC2N=C(c3ccccc3)OC2c2c1ccc1ccc(N(c3ccc(-c4ccccc4)cc3)c3ccc(-c4ccc5ccccc5c4)cc3)cc21]

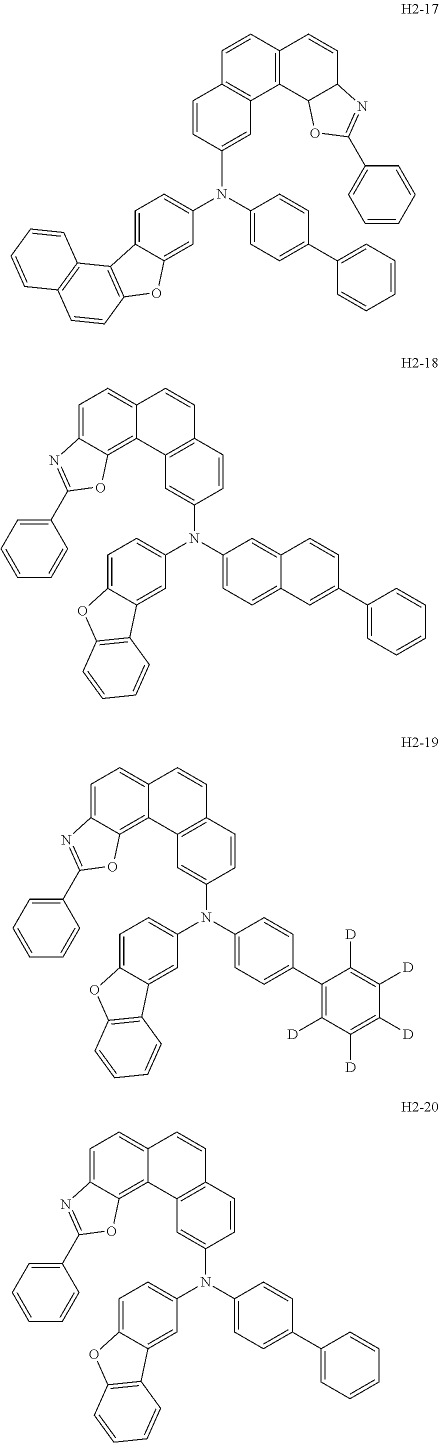

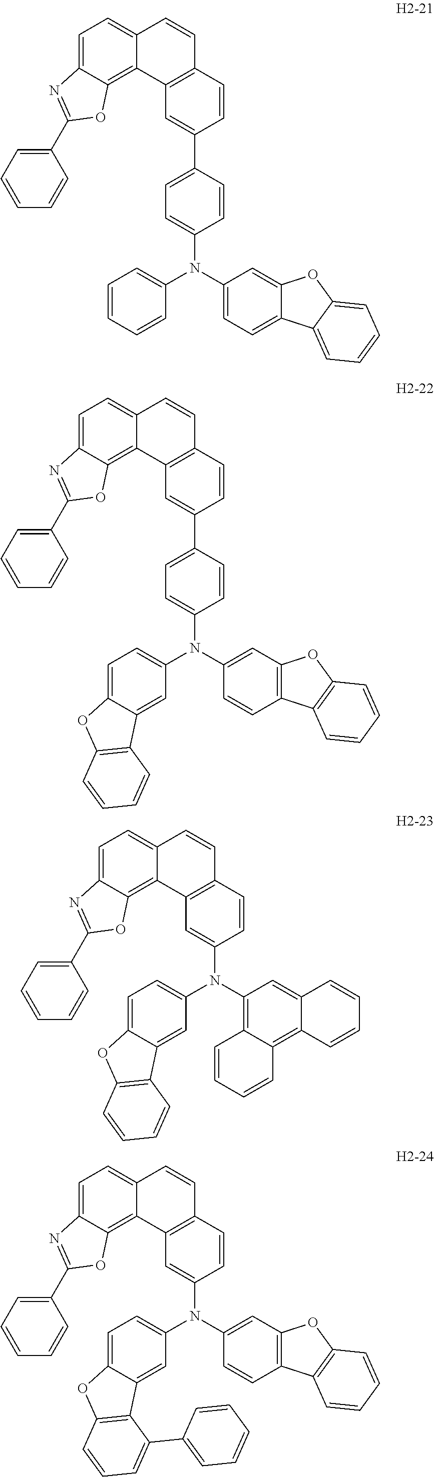

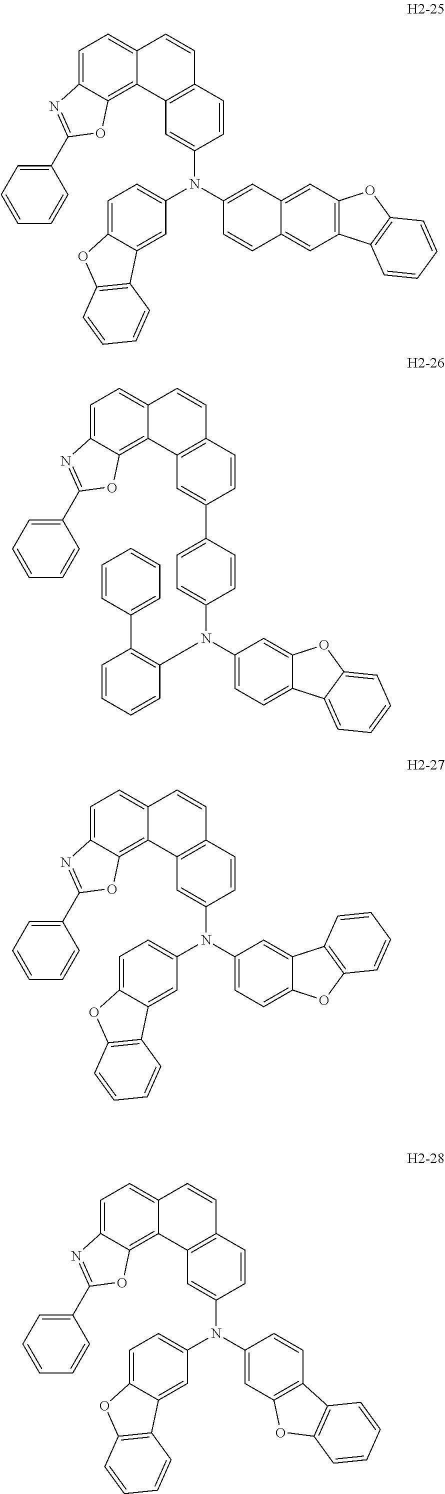 [SMILES: c1ccc(-c2nc3ccc4ccc5ccc(N(c6ccc7cc8c(cc7c6)oc6ccccc68)c6ccc7oc8ccccc8c7c6)cc5c4c3o2)cc1 ; c1ccc(-c2nc3ccc4ccc5ccc(-c6ccc(N(c7ccc8c(c7)oc7ccccc78)c7ccccc7-c7ccccc7)cc6)cc5c4c3o2)cc1 ; c1ccc(-c2nc3ccc4ccc5ccc(N(c6ccc7oc8ccccc8c7c6)c6ccc7oc8ccccc8c7c6)cc5c4c3o2)cc1 ; c1ccc(-c2nc3ccc4ccc5ccc(N(c6ccc7c(c6)oc6ccccc67)c6ccc7oc8ccccc8c7c6)cc5c4c3o2)cc1]

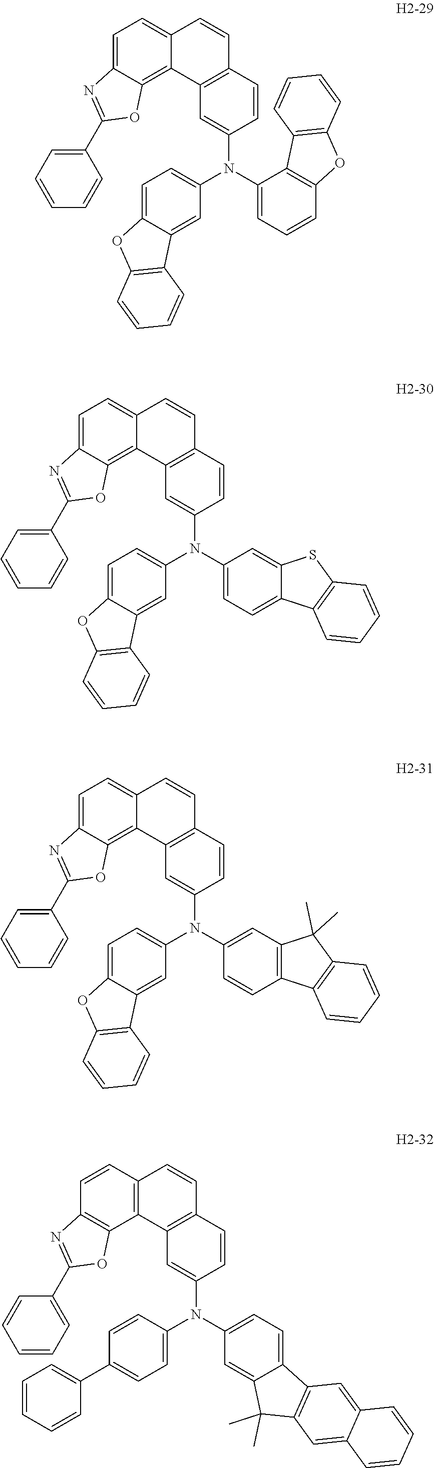 [SMILES: c1ccc(-c2nc3ccc4ccc5ccc(N(c6ccc7oc8ccccc8c7c6)c6cccc7oc8ccccc8c67)cc5c4c3o2)cc1 ; c1ccc(-c2nc3ccc4ccc5ccc(N(c6ccc7c(c6)sc6ccccc67)c6ccc7oc8ccccc8c7c6)cc5c4c3o2)cc1 ; CC1(C)c2ccccc2-c2ccc(N(c3ccc4oc5ccccc5c4c3)c3ccc4ccc5ccc6nc(-c7ccccc7)oc6c5c4c3)cc21 ; CC1(C)c2cc(N(c3ccc(-c4ccccc4)cc3)c3ccc4ccc5ccc6nc(-c7ccccc7)oc6c5c4c3)ccc2-c2cc3ccccc3cc21]

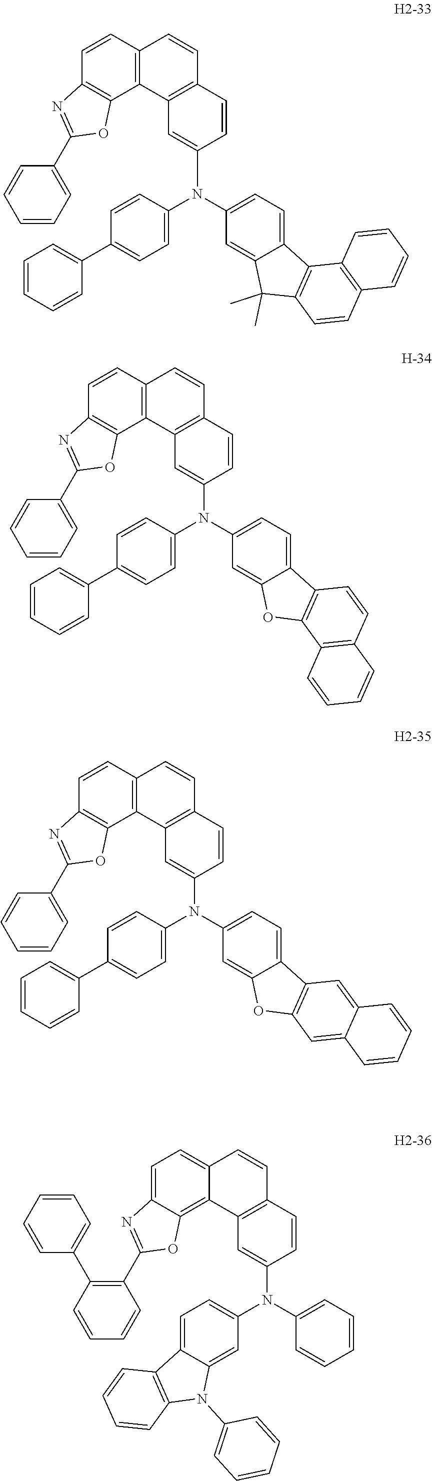 [SMILES: CC1(C)c2cc(N(c3ccc(-c4ccccc4)cc3)c3ccc4ccc5ccc6nc(-c7ccccc7)oc6c5c4c3)ccc2-c2c1ccc1ccccc21 ; c1ccc(-c2ccc(N(c3ccc4c(c3)oc3c5ccccc5ccc43)c3ccc4ccc5ccc6nc(-c7ccccc7)oc6c5c4c3)cc2)cc1 ; c1ccc(-c2ccc(N(c3ccc4c(c3)oc3cc5ccccc5cc34)c3ccc4ccc5ccc6nc(-c7ccccc7)oc6c5c4c3)cc2)cc1 ; c1ccc(-c2ccccc2-c2nc3ccc4ccc5ccc(N(c6ccccc6)c6ccc7c8ccccc8n(-c8ccccc8)c7c6)cc5c4c3o2)cc1]

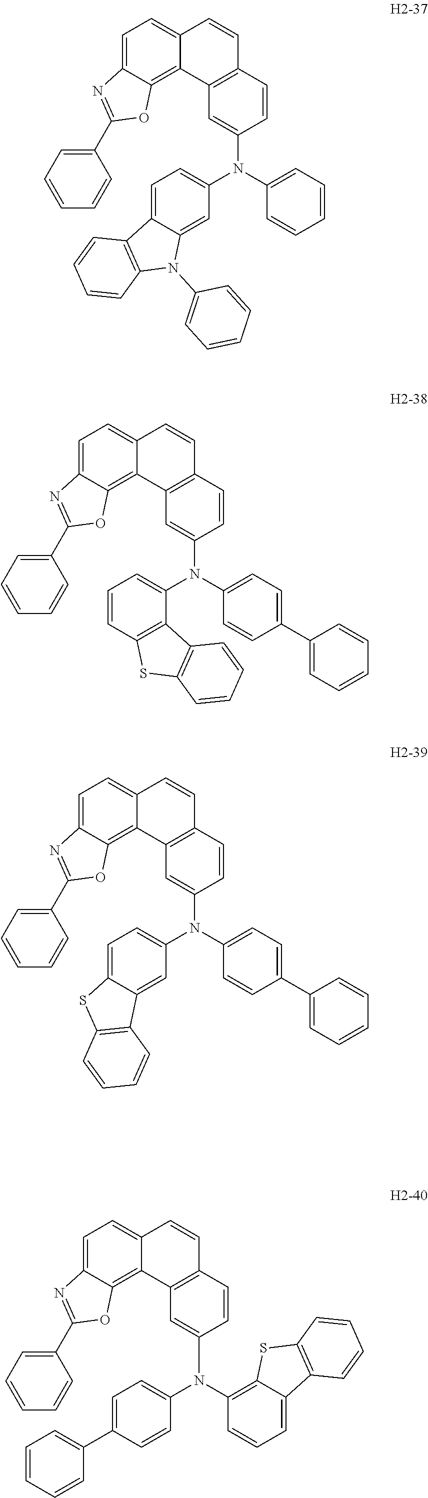 [SMILES: c1ccc(-c2nc3ccc4ccc5ccc(N(c6ccccc6)c6ccc7c8ccccc8n(-c8ccccc8)c7c6)cc5c4c3o2)cc1 ; c1ccc(-c2ccc(N(c3ccc4ccc5ccc6nc(-c7ccccc7)oc6c5c4c3)c3cccc4sc5ccccc5c34)cc2)cc1 ; c1ccc(-c2ccc(N(c3ccc4sc5ccccc5c4c3)c3ccc4ccc5ccc6nc(-c7ccccc7)oc6c5c4c3)cc2)cc1 ; c1ccc(-c2ccc(N(c3ccc4ccc5ccc6nc(-c7ccccc7)oc6c5c4c3)c3cccc4c3sc3ccccc34)cc2)cc1]

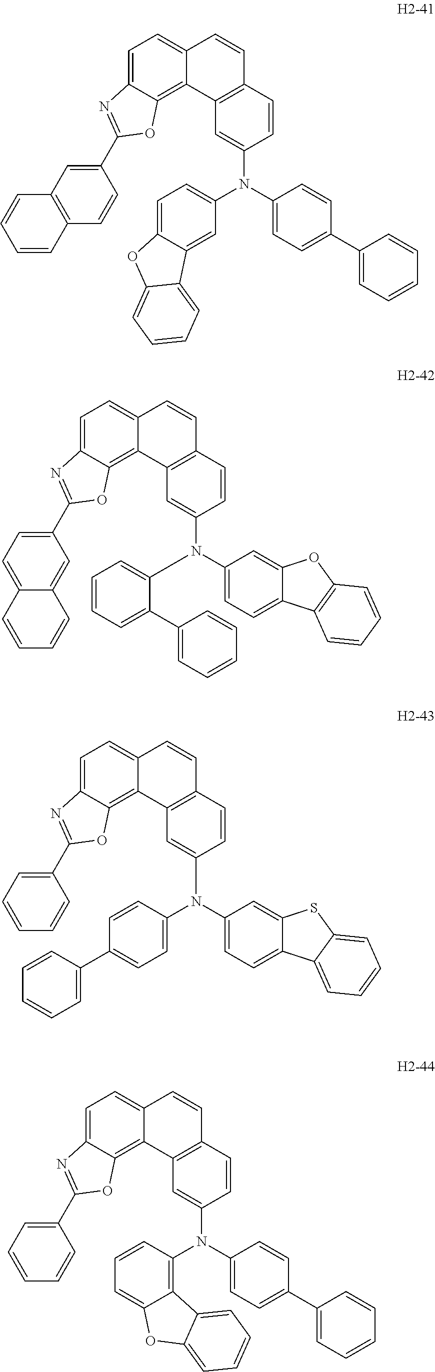 [SMILES: c1ccc(-c2ccc(N(c3ccc4oc5ccccc5c4c3)c3ccc4ccc5ccc6nc(-c7ccc8ccccc8c7)oc6c5c4c3)cc2)cc1 ; c1ccc(-c2ccccc2N(c2ccc3c(c2)oc2ccccc23)c2ccc3ccc4ccc5nc(-c6ccc7ccccc7c6)oc5c4c3c2)cc1 ; c1ccc(-c2ccc(N(c3ccc4c(c3)sc3ccccc34)c3ccc4ccc5ccc6nc(-c7ccccc7)oc6c5c4c3)cc2)cc1 ; c1ccc(-c2ccc(N(c3ccc4ccc5ccc6nc(-c7ccccc7)oc6c5c4c3)c3cccc4oc5ccccc5c34)cc2)cc1]

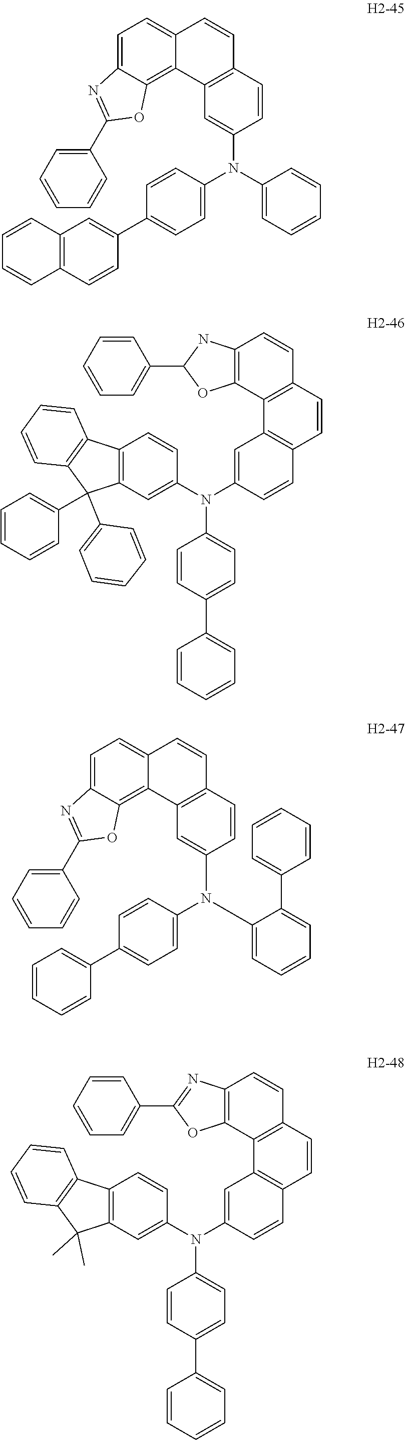

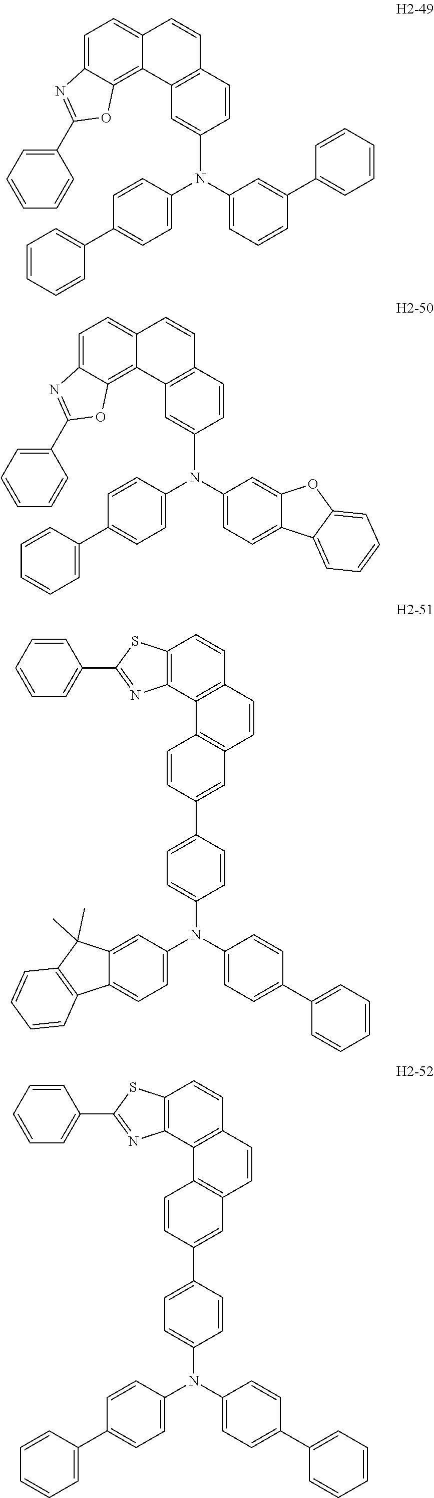

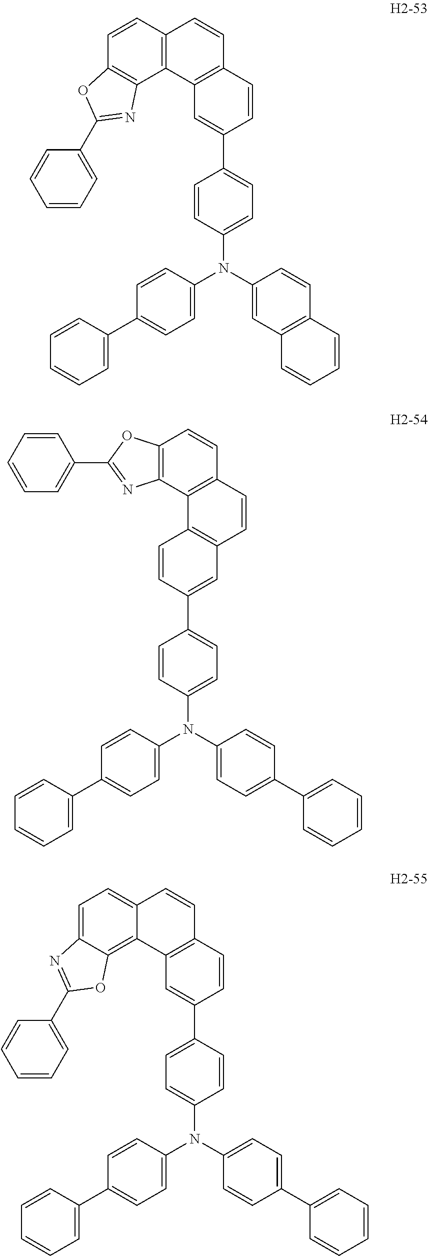 [SMILES: c1ccc(-c2ccc(N(c3ccc(-c4ccc5ccc6ccc7oc(-c8ccccc8)nc7c6c5c4)cc3)c3ccc4ccccc4c3)cc2)cc1 ; c1ccc(-c2ccc(N(c3ccc(-c4ccccc4)cc3)c3ccc(-c4ccc5c(ccc6ccc7oc(-c8ccccc8)nc7c65)c4)cc3)cc2)cc1 ; c1ccc(-c2ccc(N(c3ccc(-c4ccccc4)cc3)c3ccc(-c4ccc5ccc6ccc7nc(-c8ccccc8)oc7c6c5c4)cc3)cc2)cc1]

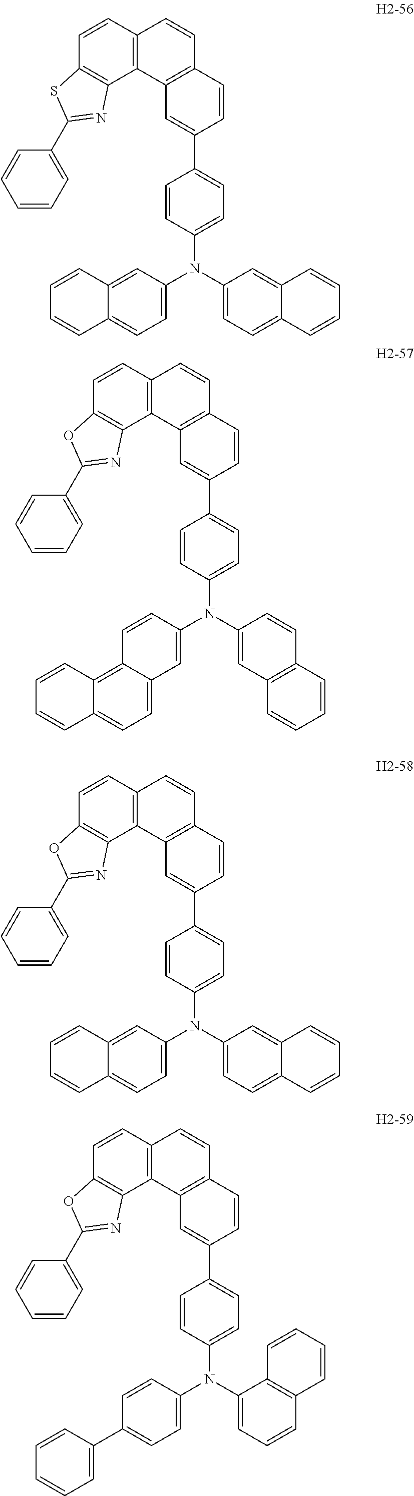

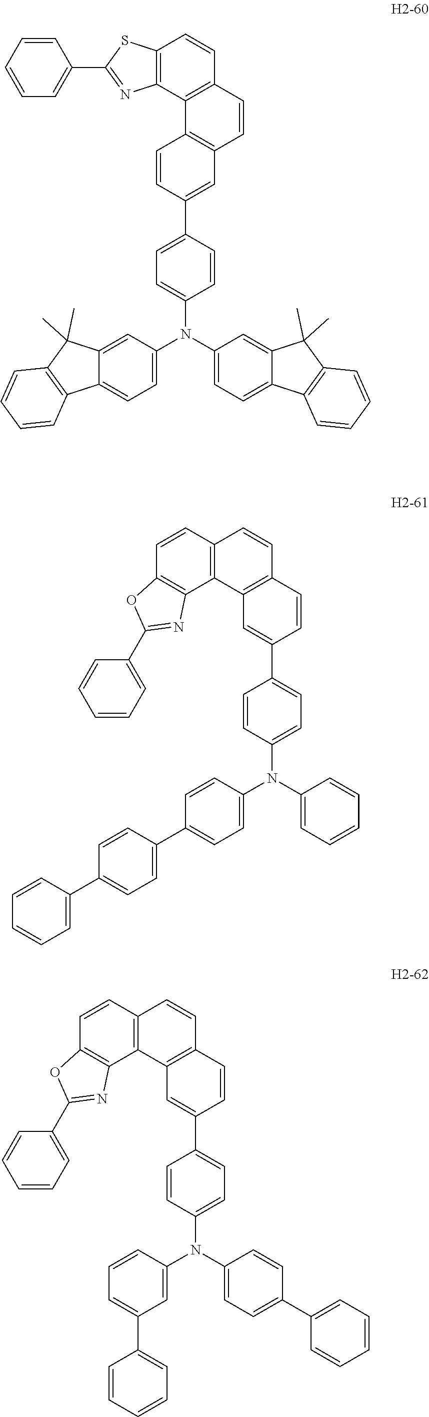 [SMILES: CC1(C)c2ccccc2-c2ccc(N(c3ccc(-c4ccc5c(ccc6ccc7sc(-c8ccccc8)nc7c65)c4)cc3)c3ccc4c(c3)C(C)(C)c3ccccc3-4)cc21 ; c1ccc(-c2ccc(-c3ccc(N(c4ccccc4)c4ccc(-c5ccc6ccc7ccc8oc(-c9ccccc9)nc8c7c6c5)cc4)cc3)cc2)cc1 ; c1ccc(-c2ccc(N(c3ccc(-c4ccc5ccc6ccc7oc(-c8ccccc8)nc7c6c5c4)cc3)c3cccc(-c4ccccc4)c3)cc2)cc1]

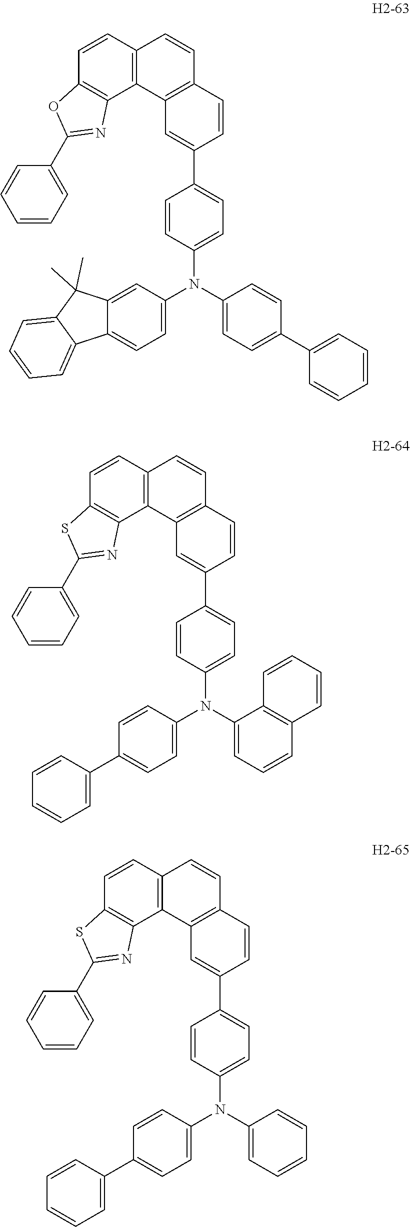 [SMILES: CC1(C)c2ccccc2-c2ccc(N(c3ccc(-c4ccccc4)cc3)c3ccc(-c4ccc5ccc6ccc7oc(-c8ccccc8)nc7c6c5c4)cc3)cc21 ; c1ccc(-c2ccc(N(c3ccc(-c4ccc5ccc6ccc7sc(-c8ccccc8)nc7c6c5c4)cc3)c3cccc4ccccc34)cc2)cc1 ; c1ccc(-c2ccc(N(c3ccccc3)c3ccc(-c4ccc5ccc6ccc7sc(-c8ccccc8)nc7c6c5c4)cc3)cc2)cc1]

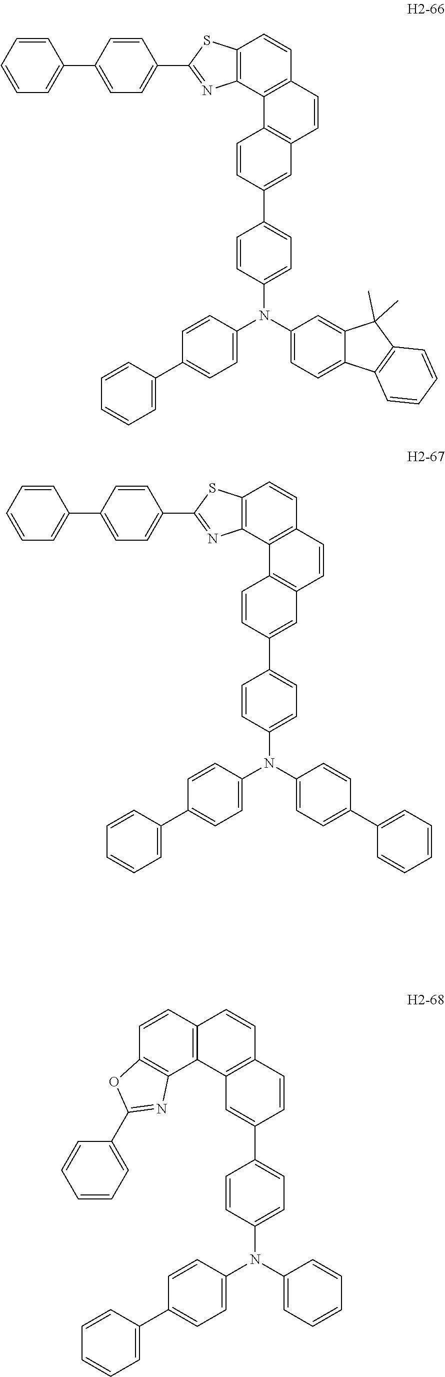 [SMILES: CC1(C)c2ccccc2-c2ccc(N(c3ccc(-c4ccccc4)cc3)c3ccc(-c4ccc5c(ccc6ccc7sc(-c8ccc(-c9ccccc9)cc8)nc7c65)c4)cc3)cc21 ; c1ccc(-c2ccc(-c3nc4c(ccc5ccc6cc(-c7ccc(N(c8ccc(-c9ccccc9)cc8)c8ccc(-c9ccccc9)cc8)cc7)ccc6c54)s3)cc2)cc1 ; c1ccc(-c2ccc(N(c3ccccc3)c3ccc(-c4ccc5ccc6ccc7oc(-c8ccccc8)nc7c6c5c4)cc3)cc2)cc1]

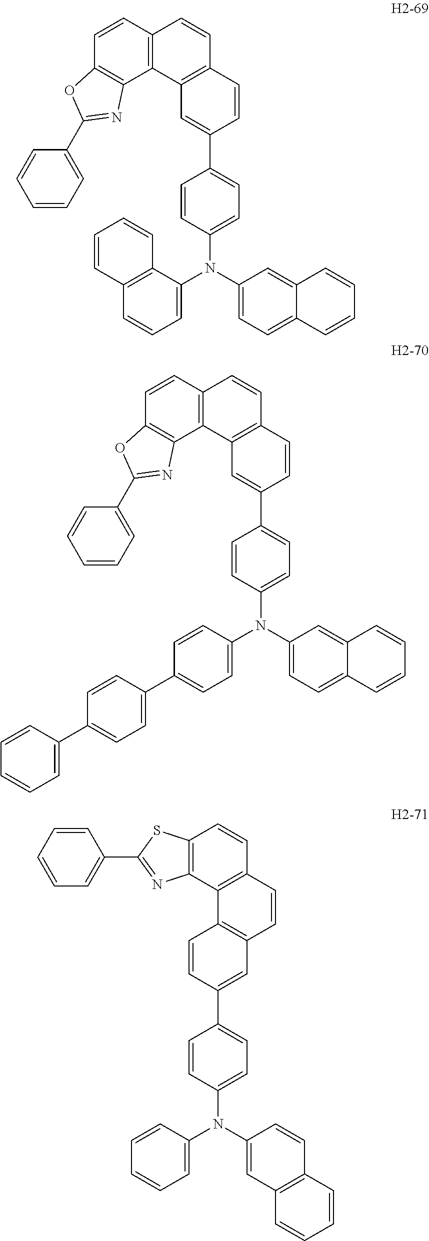

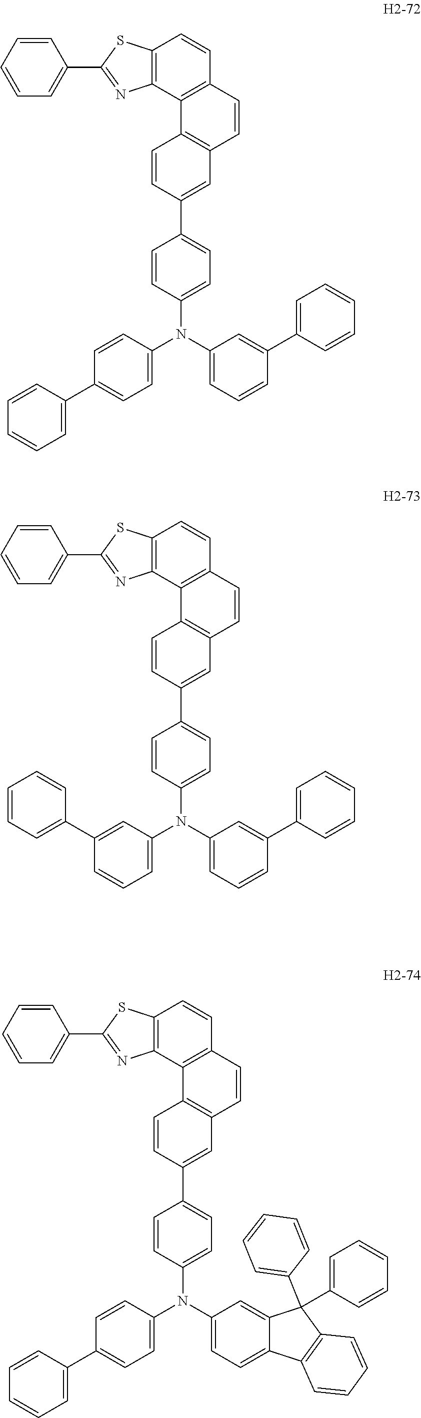 [SMILES: c1ccc(-c2ccc(N(c3ccc(-c4ccc5c(ccc6ccc7sc(-c8ccccc8)nc7c65)c4)cc3)c3cccc(-c4ccccc4)c3)cc2)cc1 ; c1ccc(-c2cccc(N(c3ccc(-c4ccc5c(ccc6ccc7sc(-c8ccccc8)nc7c65)c4)cc3)c3cccc(-c4ccccc4)c3)c2)cc1 ; c1ccc(-c2ccc(N(c3ccc(-c4ccc5c(ccc6ccc7sc(-c8ccccc8)nc7c65)c4)cc3)c3ccc4c(c3)C(c3ccccc3)(c3ccccc3)c3ccccc3-4)cc2)cc1]

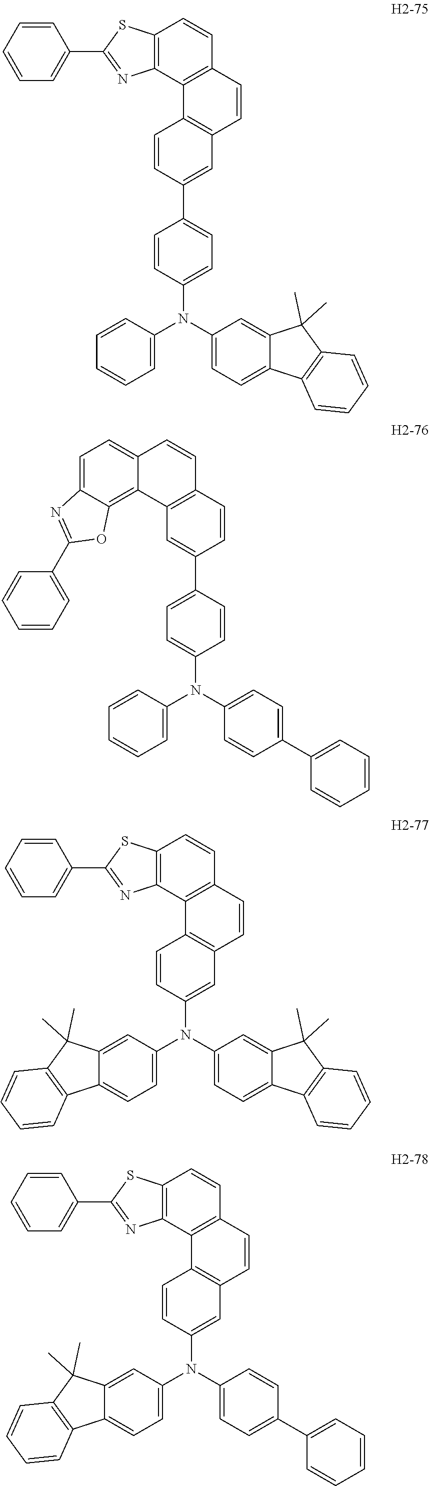

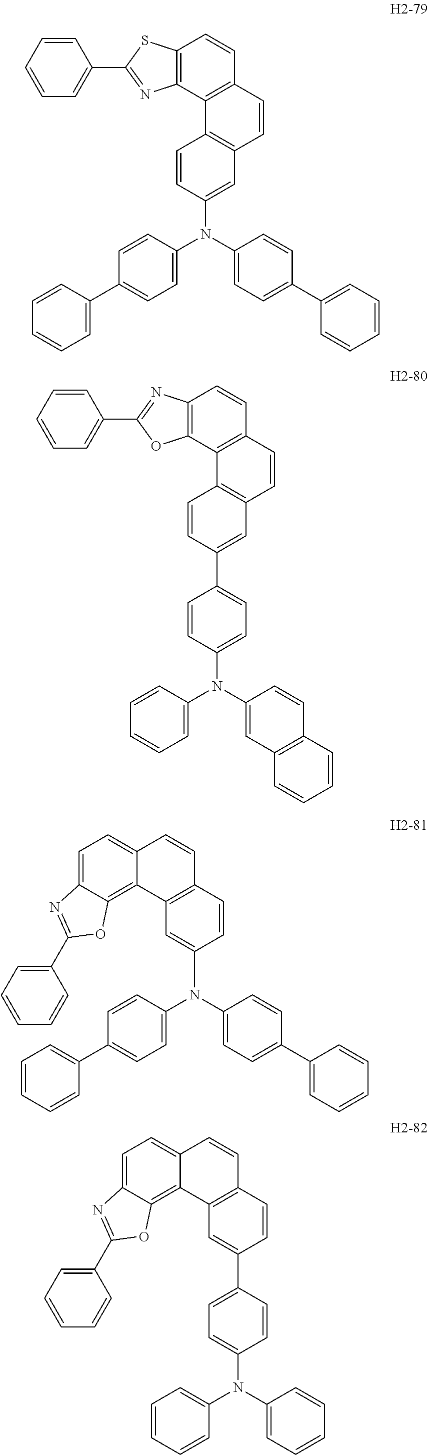

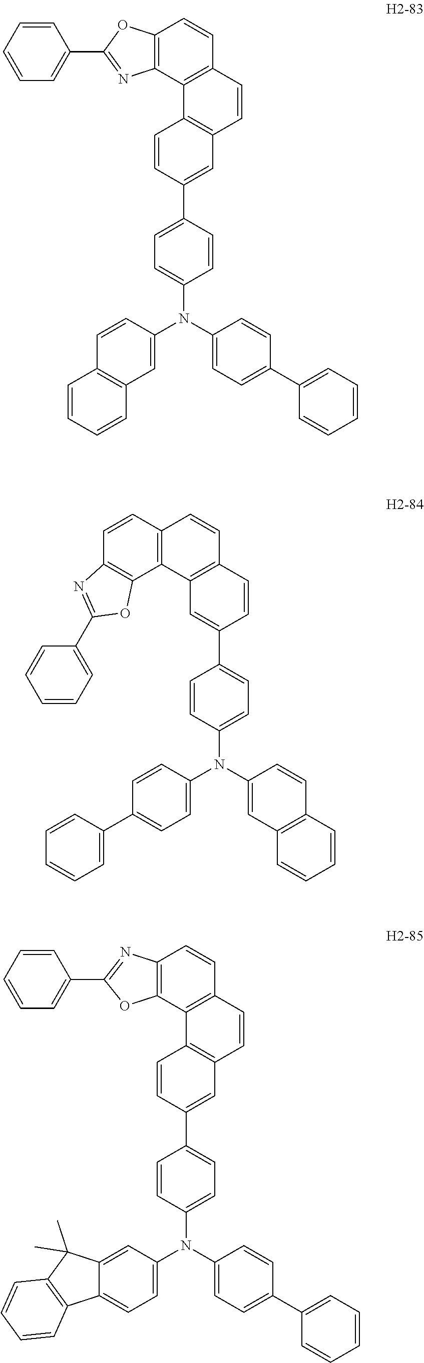 [SMILES: c1ccc(-c2ccc(N(c3ccc(-c4ccc5c(ccc6ccc7oc(-c8ccccc8)nc7c65)c4)cc3)c3ccc4ccccc4c3)cc2)cc1 ; c1ccc(-c2ccc(N(c3ccc(-c4ccc5ccc6ccc7nc(-c8ccccc8)oc7c6c5c4)cc3)c3ccc4ccccc4c3)cc2)cc1 ; CC1(C)c2ccccc2-c2ccc(N(c3ccc(-c4ccccc4)cc3)c3ccc(-c4ccc5c(ccc6ccc7nc(-c8ccccc8)oc7c65)c4)cc3)cc21]

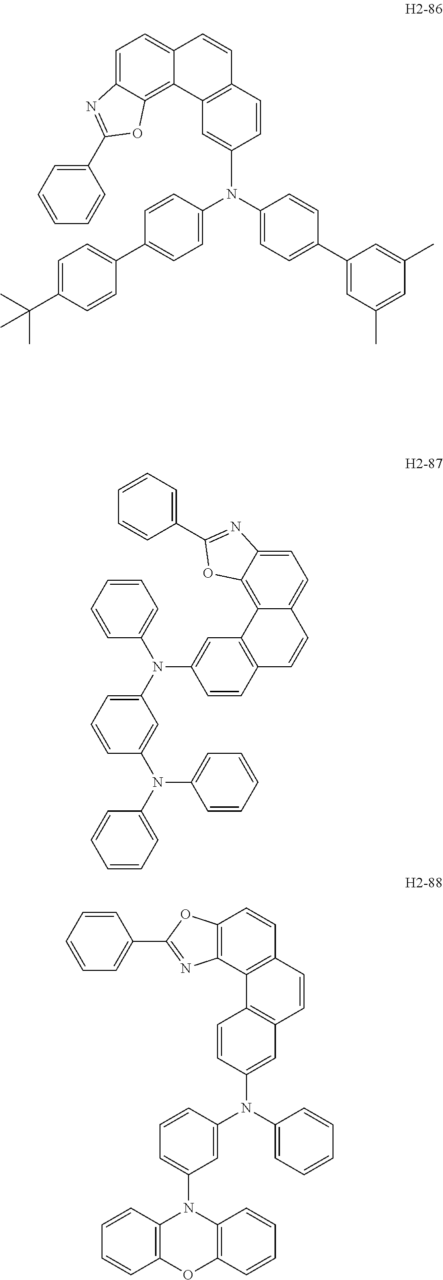

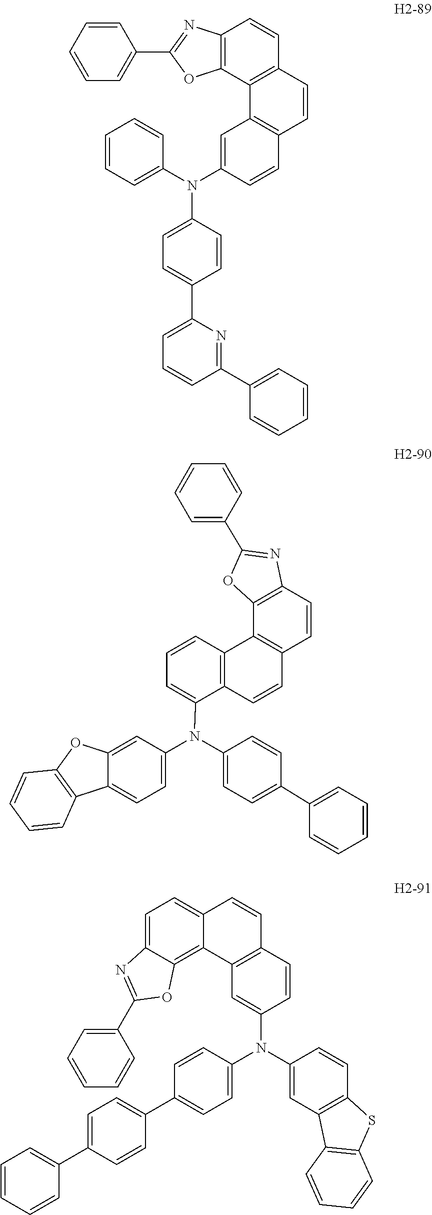 [SMILES: c1ccc(-c2cccc(-c3ccc(N(c4ccccc4)c4ccc5ccc6ccc7nc(-c8ccccc8)oc7c6c5c4)cc3)n2)cc1 ; c1ccc(-c2ccc(N(c3ccc4c(c3)oc3ccccc34)c3cccc4c3ccc3ccc5nc(-c6ccccc6)oc5c34)cc2)cc1 ; c1ccc(-c2ccc(-c3ccc(N(c4ccc5sc6ccccc6c5c4)c4ccc5ccc6ccc7nc(-c8ccccc8)oc7c6c5c4)cc3)cc2)cc1]

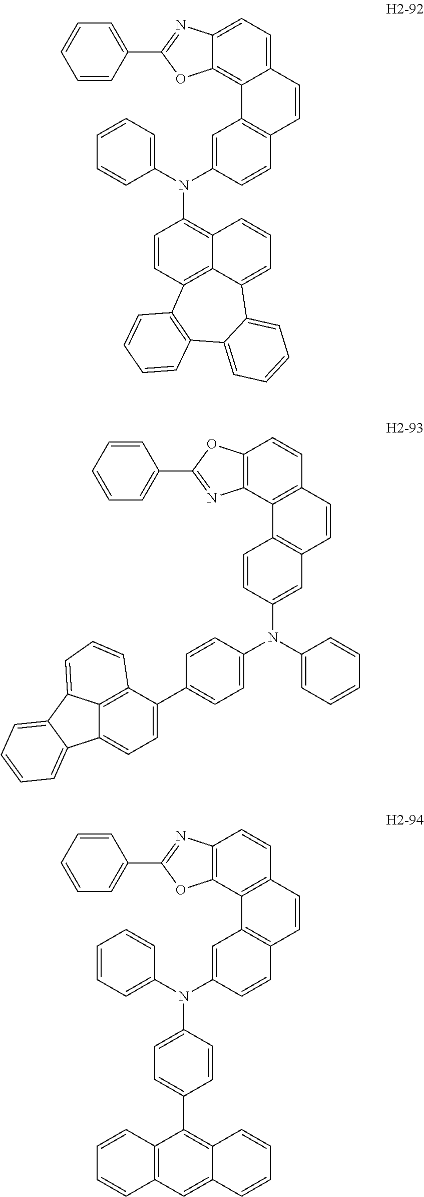 [SMILES: c1ccc(-c2nc3ccc4ccc5ccc(N(c6ccccc6)c6ccc7c8c(cccc68)-c6ccccc6-c6ccccc6-7)cc5c4c3o2)cc1 ; c1ccc(-c2nc3c(ccc4ccc5cc(N(c6ccccc6)c6ccc(-c7ccc8c9c(cccc79)-c7ccccc7-8)cc6)ccc5c43)o2)cc1 ; c1ccc(-c2nc3ccc4ccc5ccc(N(c6ccccc6)c6ccc(-c7c8ccccc8cc8ccccc78)cc6)cc5c4c3o2)cc1]

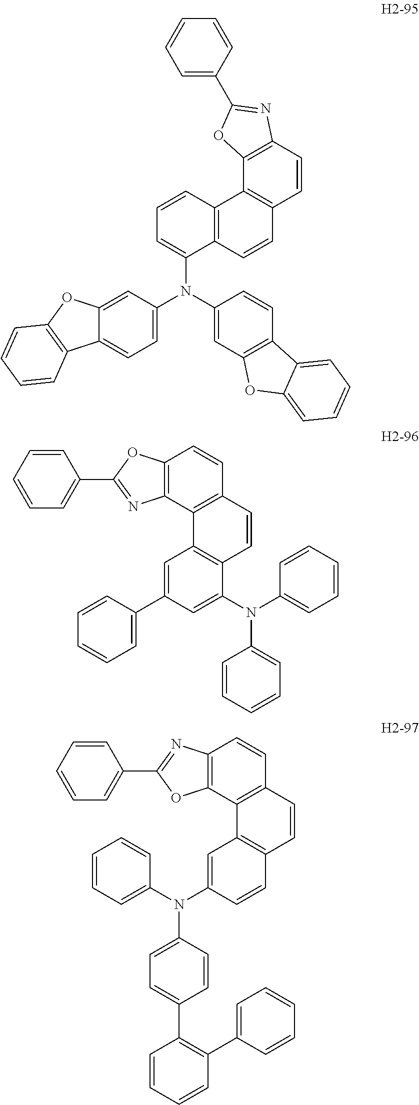

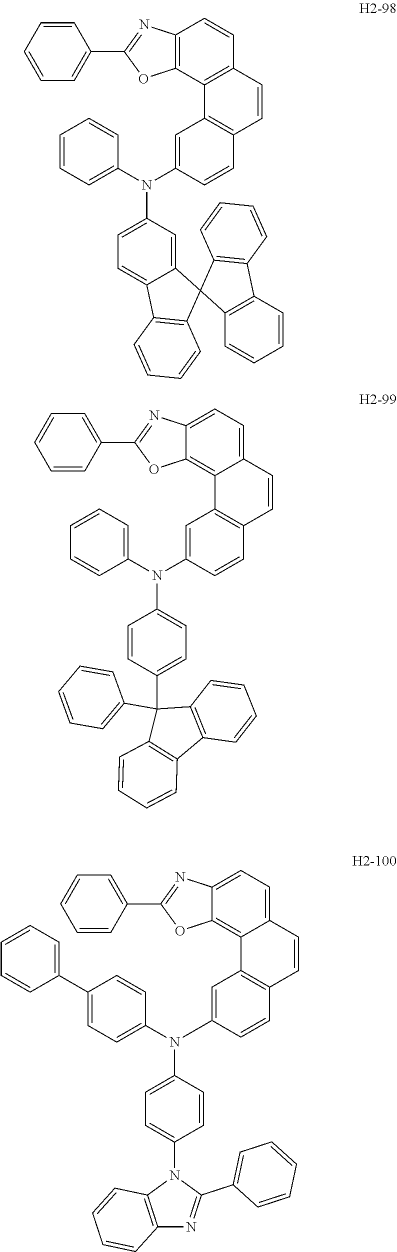

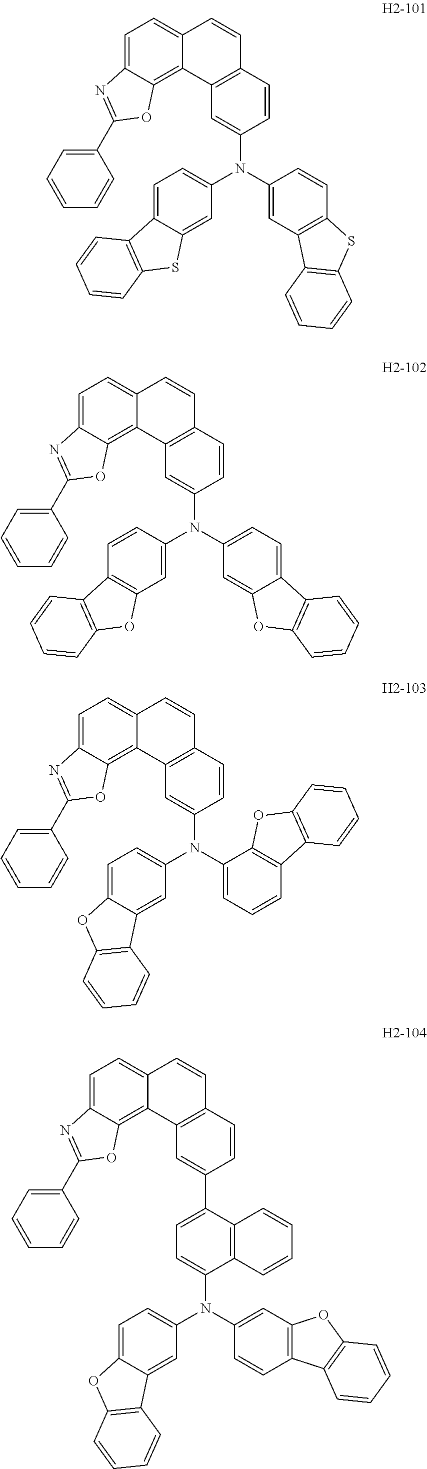 [SMILES: c1ccc(-c2nc3ccc4ccc5ccc(N(c6ccc7c(c6)sc6ccccc67)c6ccc7sc8ccccc8c7c6)cc5c4c3o2)cc1 ; c1ccc(-c2nc3ccc4ccc5ccc(N(c6ccc7c(c6)oc6ccccc67)c6ccc7c(c6)oc6ccccc67)cc5c4c3o2)cc1 ; c1ccc(-c2nc3ccc4ccc5ccc(N(c6ccc7oc8ccccc8c7c6)c6cccc7c6oc6ccccc67)cc5c4c3o2)cc1 ; c1ccc(-c2nc3ccc4ccc5ccc(-c6ccc(N(c7ccc8c(c7)oc7ccccc78)c7ccc8oc9ccccc9c8c7)c7ccccc67)cc5c4c3o2)cc1]

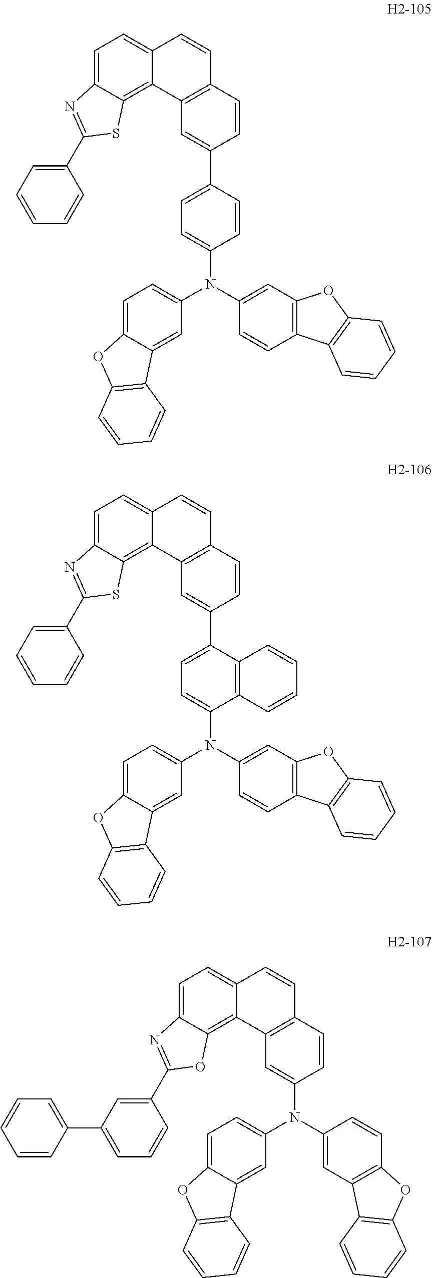 [SMILES: c1ccc(-c2nc3ccc4ccc5ccc(-c6ccc(N(c7ccc8c(c7)oc7ccccc78)c7ccc8oc9ccccc9c8c7)cc6)cc5c4c3s2)cc1 ; c1ccc(-c2nc3ccc4ccc5ccc(-c6ccc(N(c7ccc8c(c7)oc7ccccc78)c7ccc8oc9ccccc9c8c7)c7ccccc67)cc5c4c3s2)cc1 ; c1ccc(-c2cccc(-c3nc4ccc5ccc6ccc(N(c7ccc8oc9ccccc9c8c7)c7ccc8oc9ccccc9c8c7)cc6c5c4o3)c2)cc1]

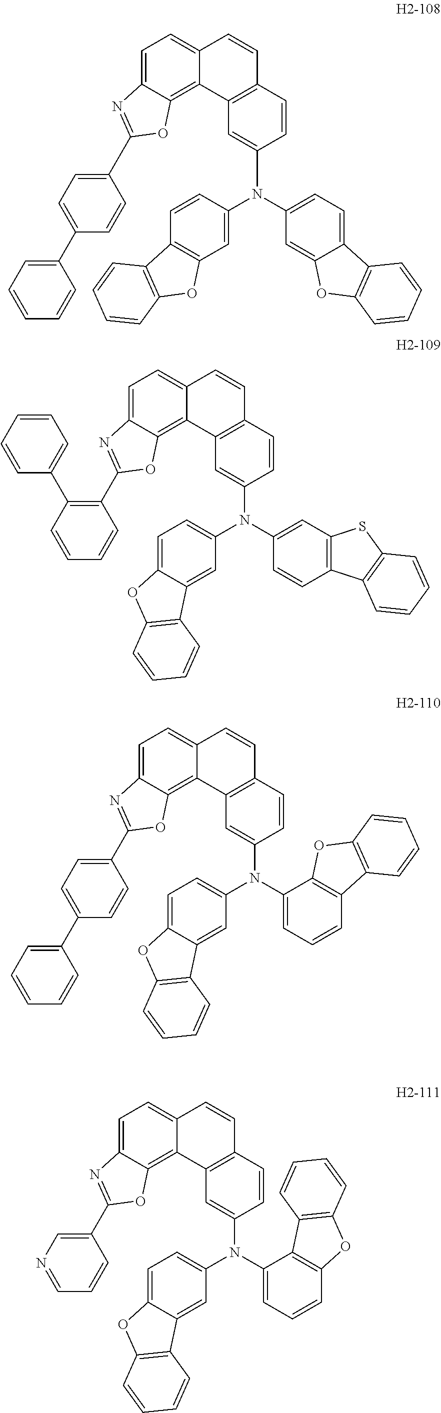 [SMILES: c1ccc(-c2ccc(-c3nc4ccc5ccc6ccc(N(c7ccc8c(c7)oc7ccccc78)c7ccc8c(c7)oc7ccccc78)cc6c5c4o3)cc2)cc1 ; c1ccc(-c2ccccc2-c2nc3ccc4ccc5ccc(N(c6ccc7c(c6)sc6ccccc67)c6ccc7oc8ccccc8c7c6)cc5c4c3o2)cc1 ; c1ccc(-c2ccc(-c3nc4ccc5ccc6ccc(N(c7ccc8oc9ccccc9c8c7)c7cccc8c7oc7ccccc78)cc6c5c4o3)cc2)cc1 ; c1cncc(-c2nc3ccc4ccc5ccc(N(c6ccc7oc8ccccc8c7c6)c6cccc7oc8ccccc8c67)cc5c4c3o2)c1]

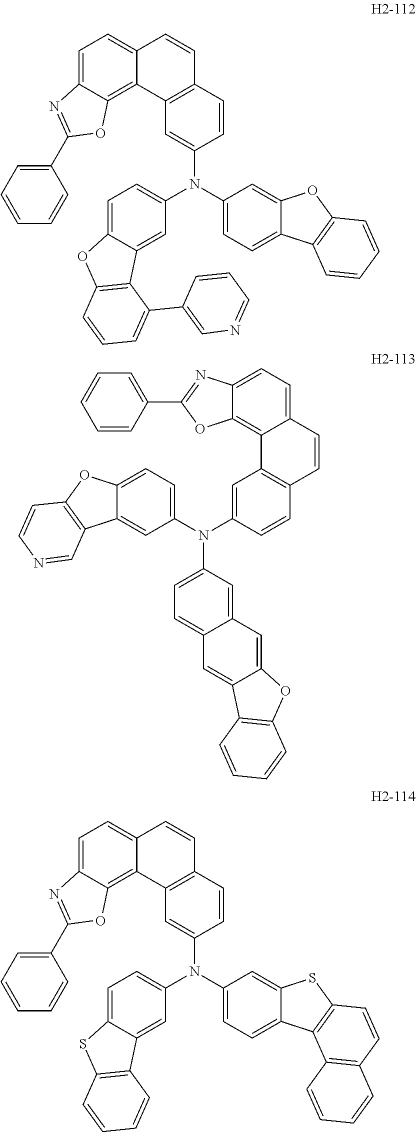

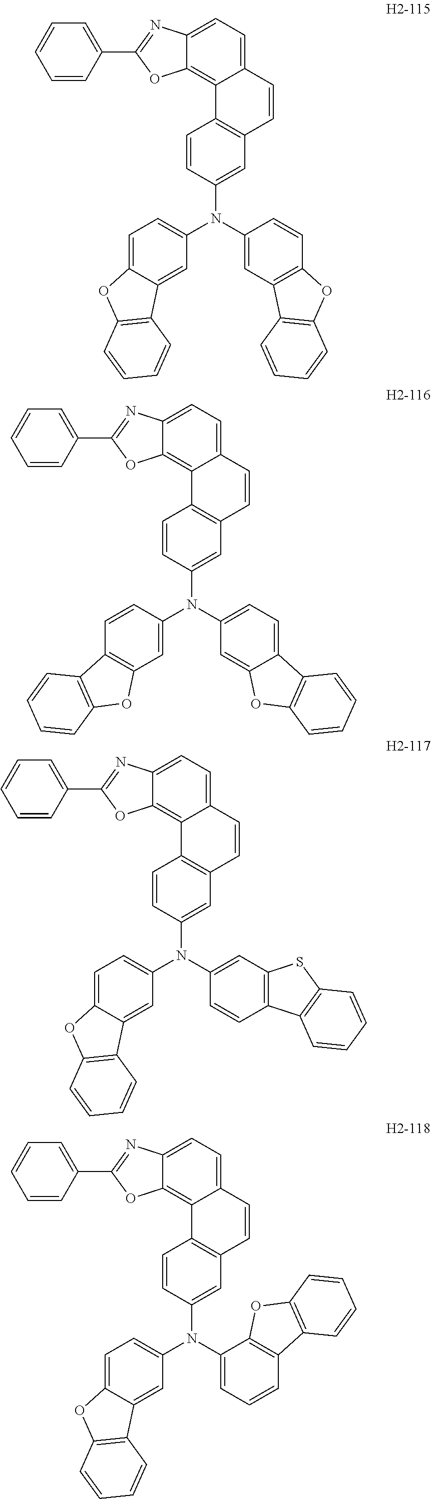

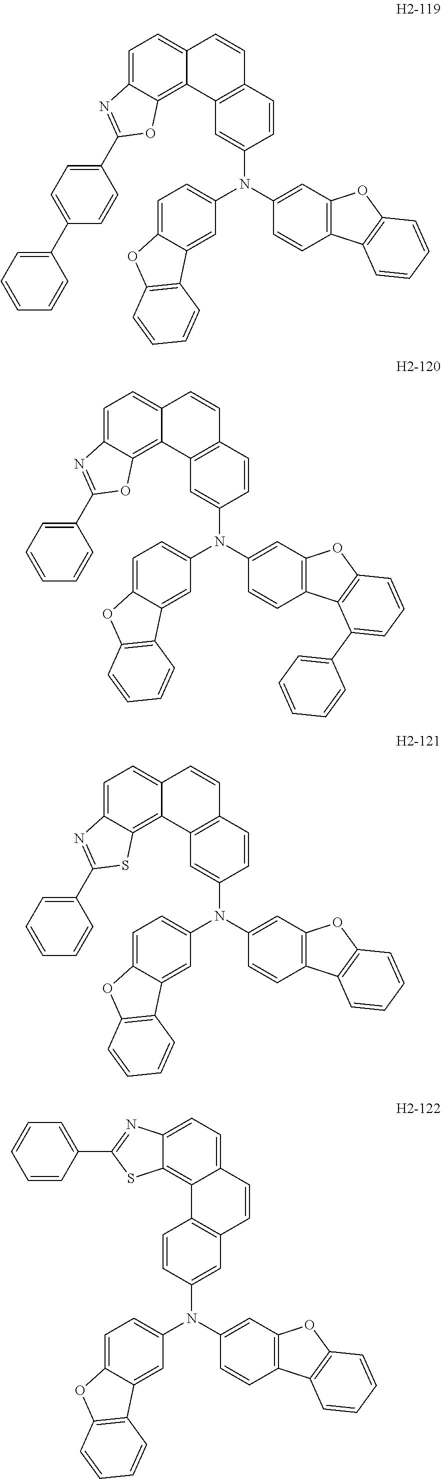 [SMILES: c1ccc(-c2ccc(-c3nc4ccc5ccc6ccc(N(c7ccc8c(c7)oc7ccccc78)c7ccc8oc9ccccc9c8c7)cc6c5c4o3)cc2)cc1 ; c1ccc(-c2nc3ccc4ccc5ccc(N(c6ccc7c(c6)oc6cccc(-c8ccccc8)c67)c6ccc7oc8ccccc8c7c6)cc5c4c3o2)cc1 ; c1ccc(-c2nc3ccc4ccc5ccc(N(c6ccc7c(c6)oc6ccccc67)c6ccc7oc8ccccc8c7c6)cc5c4c3s2)cc1 ; c1ccc(-c2nc3ccc4ccc5cc(N(c6ccc7c(c6)oc6ccccc67)c6ccc7oc8ccccc8c7c6)ccc5c4c3s2)cc1]

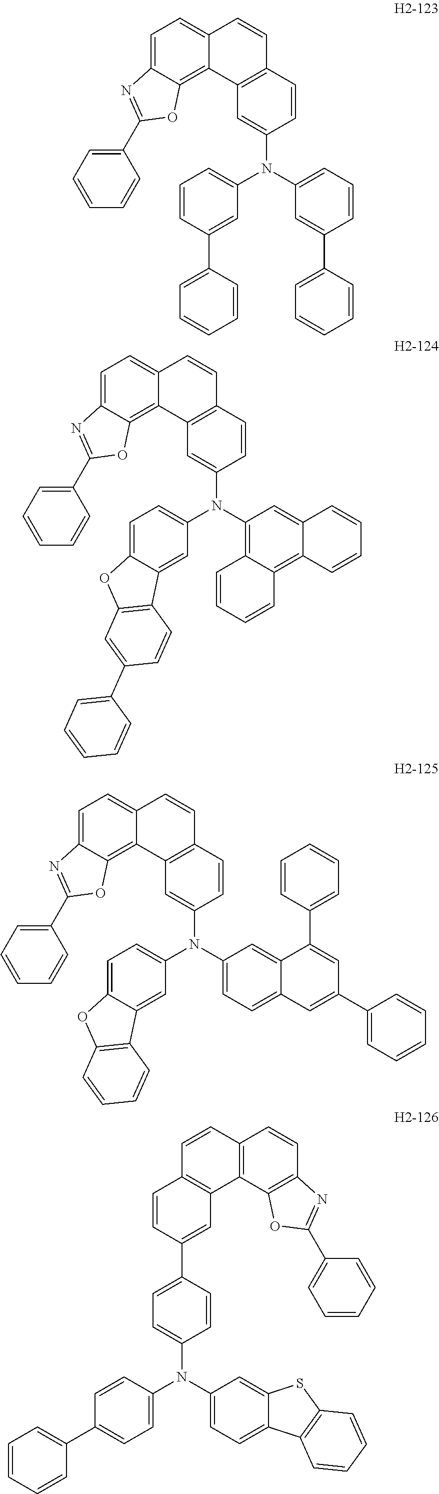

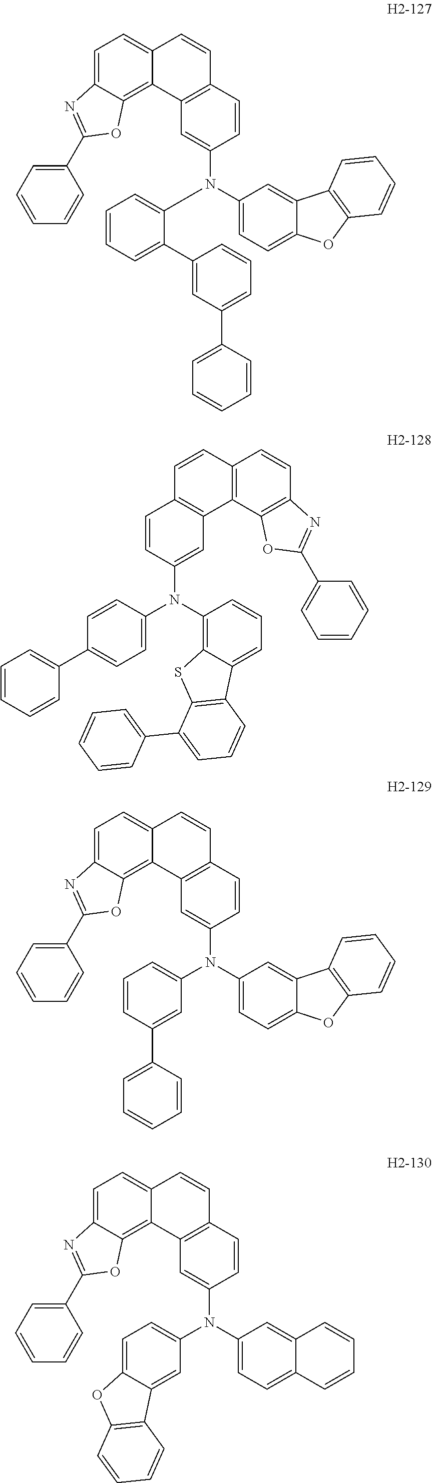

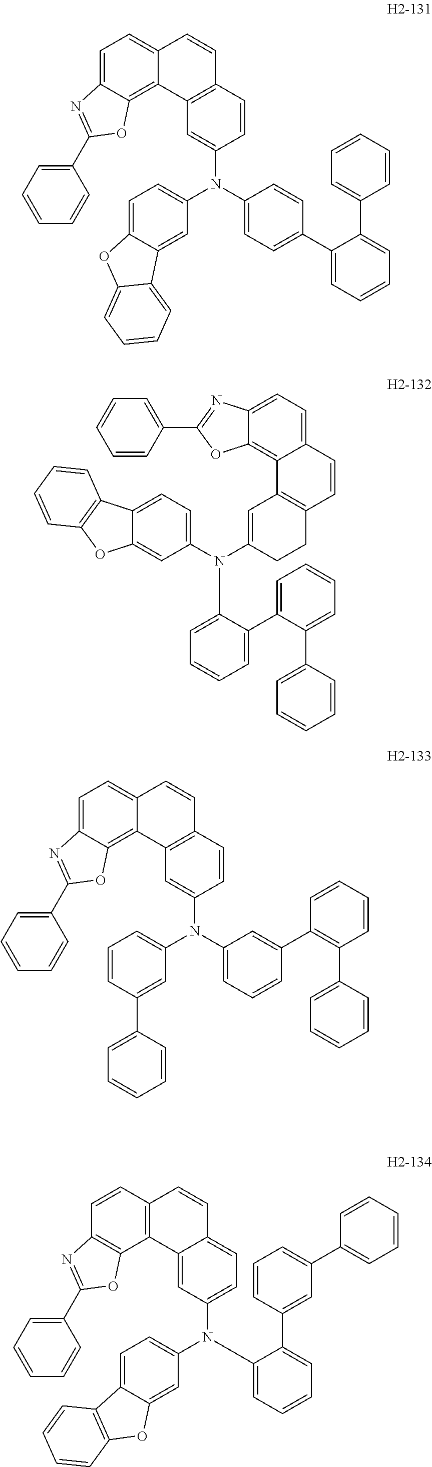 [SMILES: c1ccc(-c2nc3ccc4ccc5ccc(N(c6ccc(-c7ccccc7-c7ccccc7)cc6)c6ccc7oc8ccccc8c7c6)cc5c4c3o2)cc1 ; C1=C(N(c2ccc3c(c2)oc2ccccc23)c2ccccc2-c2ccccc2-c2ccccc2)CCc2ccc3ccc4nc(-c5ccccc5)oc4c3c21 ; c1ccc(-c2cccc(N(c3cccc(-c4ccccc4-c4ccccc4)c3)c3ccc4ccc5ccc6nc(-c7ccccc7)oc6c5c4c3)c2)cc1 ; c1ccc(-c2cccc(-c3ccccc3N(c3ccc4c(c3)oc3ccccc34)c3ccc4ccc5ccc6nc(-c7ccccc7)oc6c5c4c3)c2)cc1]

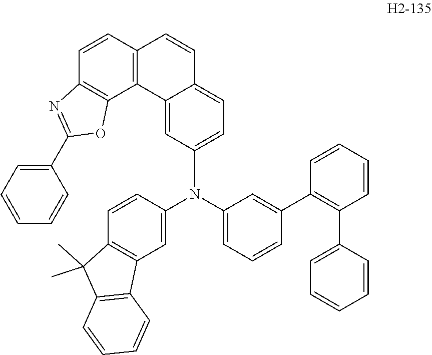 [SMILES: CC1(C)c2ccccc2-c2cc(N(c3cccc(-c4ccccc4-c4ccccc4)c3)c3ccc4ccc5ccc6nc(-c7ccccc7)oc6c5c4c3)ccc21]

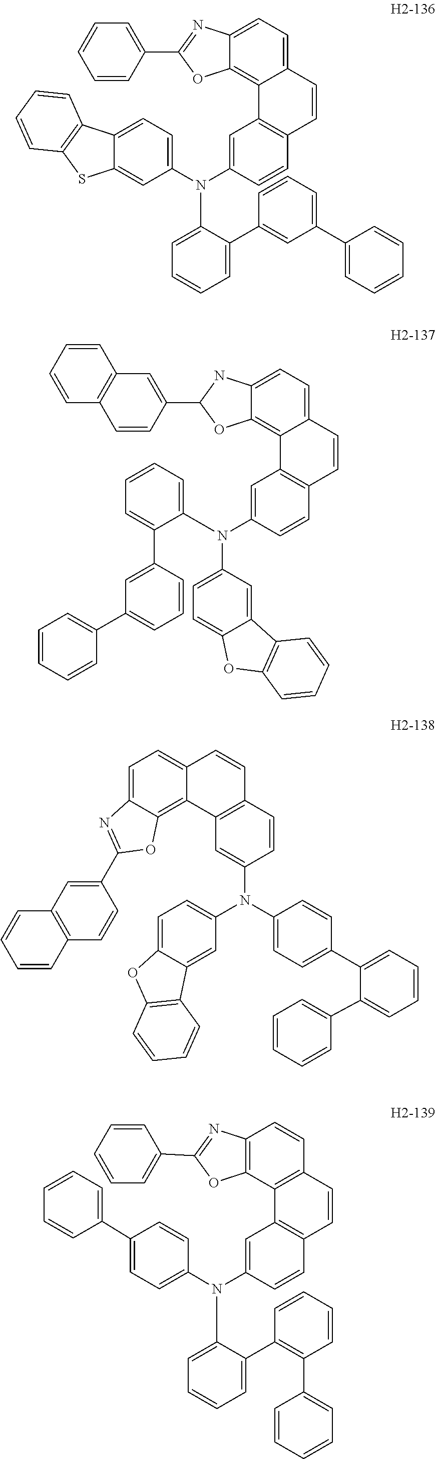

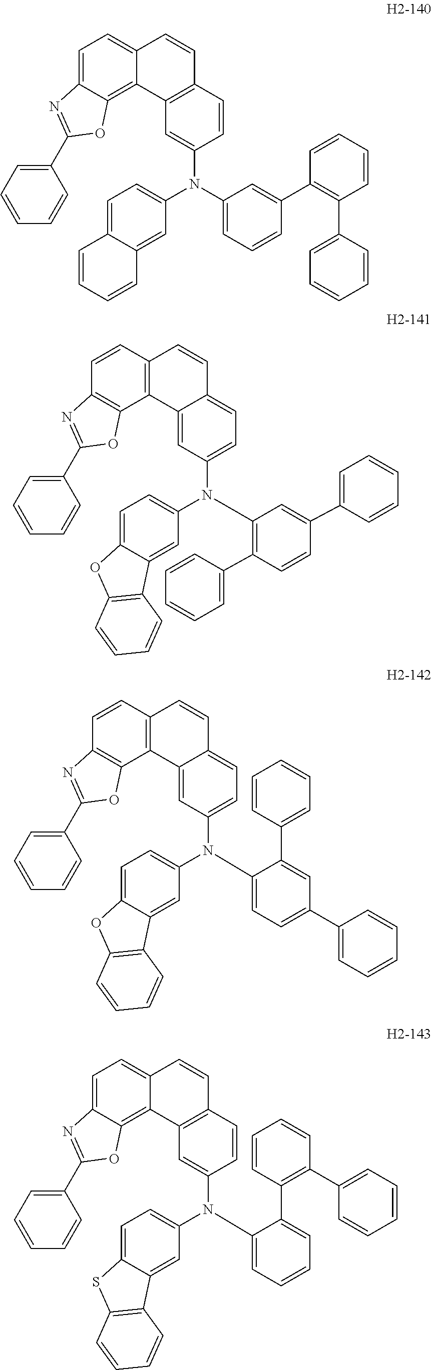

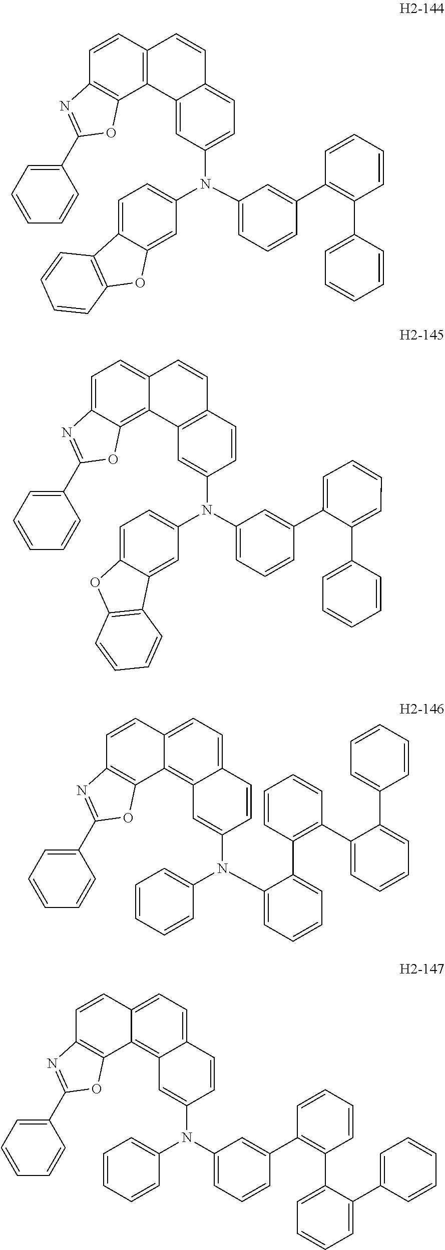 [SMILES: c1ccc(-c2nc3ccc4ccc5ccc(N(c6cccc(-c7ccccc7-c7ccccc7)c6)c6ccc7c(c6)oc6ccccc67)cc5c4c3o2)cc1 ; c1ccc(-c2nc3ccc4ccc5ccc(N(c6cccc(-c7ccccc7-c7ccccc7)c6)c6ccc7oc8ccccc8c7c6)cc5c4c3o2)cc1 ; c1ccc(-c2nc3ccc4ccc5ccc(N(c6ccccc6)c6ccccc6-c6ccccc6-c6ccccc6-c6ccccc6)cc5c4c3o2)cc1 ; c1ccc(-c2nc3ccc4ccc5ccc(N(c6ccccc6)c6cccc(-c7ccccc7-c7ccccc7-c7ccccc7)c6)cc5c4c3o2)cc1]

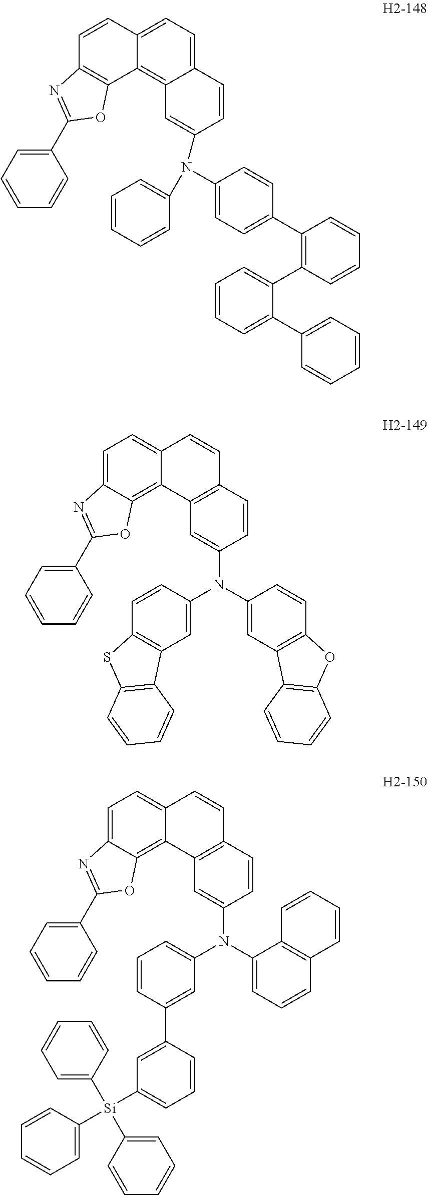 [SMILES: c1ccc(-c2nc3ccc4ccc5ccc(N(c6ccccc6)c6ccc(-c7ccccc7-c7ccccc7-c7ccccc7)cc6)cc5c4c3o2)cc1 ; c1ccc(-c2nc3ccc4ccc5ccc(N(c6ccc7oc8ccccc8c7c6)c6ccc7sc8ccccc8c7c6)cc5c4c3o2)cc1 ; c1ccc(-c2nc3ccc4ccc5ccc(N(c6cccc(-c7cccc([Si](c8ccccc8)(c8ccccc8)c8ccccc8)c7)c6)c6cccc7ccccc67)cc5c4c3o2)cc1]

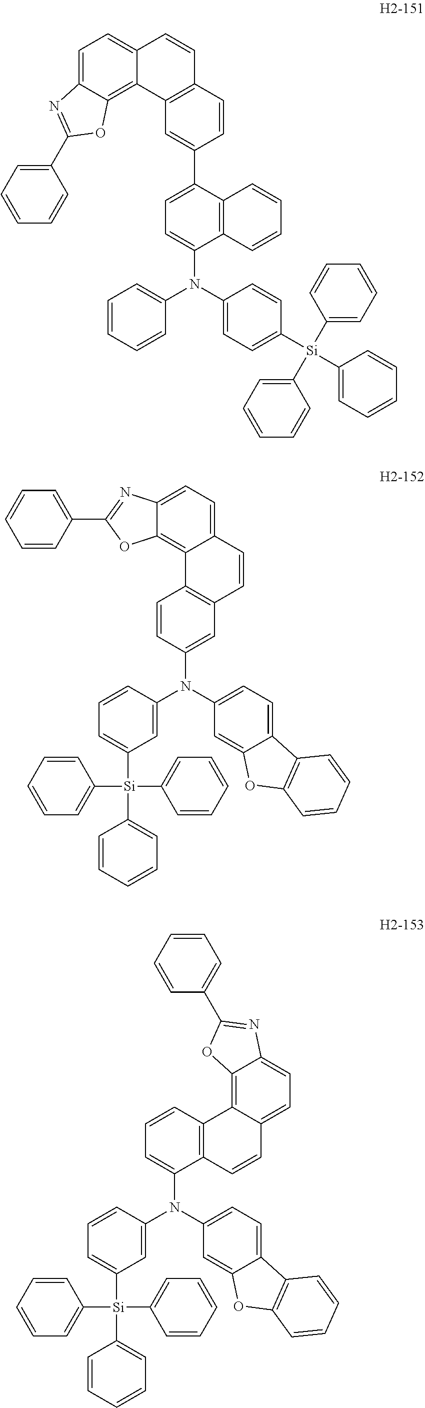 [SMILES: c1ccc(-c2nc3ccc4ccc5ccc(-c6ccc(N(c7ccccc7)c7ccc([Si](c8ccccc8)(c8ccccc8)c8ccccc8)cc7)c7ccccc67)cc5c4c3o2)cc1 ; c1ccc(-c2nc3ccc4ccc5cc(N(c6cccc([Si](c7ccccc7)(c7ccccc7)c7ccccc7)c6)c6ccc7c(c6)oc6ccccc67)ccc5c4c3o2)cc1 ; c1ccc(-c2nc3ccc4ccc5c(N(c6cccc([Si](c7ccccc7)(c7ccccc7)c7ccccc7)c6)c6ccc7c(c6)oc6ccccc67)cccc5c4c3o2)cc1]

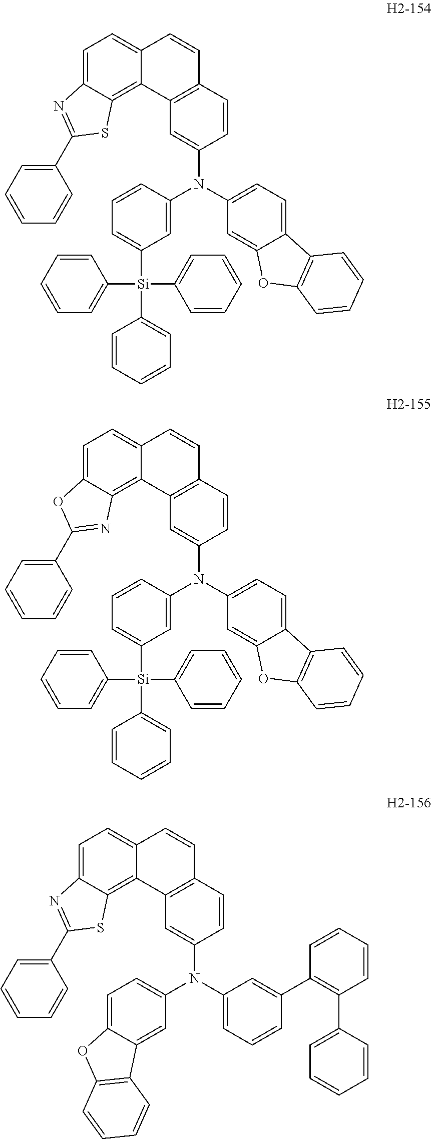 [SMILES: c1ccc(-c2nc3ccc4ccc5ccc(N(c6cccc([Si](c7ccccc7)(c7ccccc7)c7ccccc7)c6)c6ccc7c(c6)oc6ccccc67)cc5c4c3s2)cc1 ; c1ccc(-c2nc3c(ccc4ccc5ccc(N(c6cccc([Si](c7ccccc7)(c7ccccc7)c7ccccc7)c6)c6ccc7c(c6)oc6ccccc67)cc5c43)o2)cc1 ; c1ccc(-c2nc3ccc4ccc5ccc(N(c6cccc(-c7ccccc7-c7ccccc7)c6)c6ccc7oc8ccccc8c7c6)cc5c4c3s2)cc1]

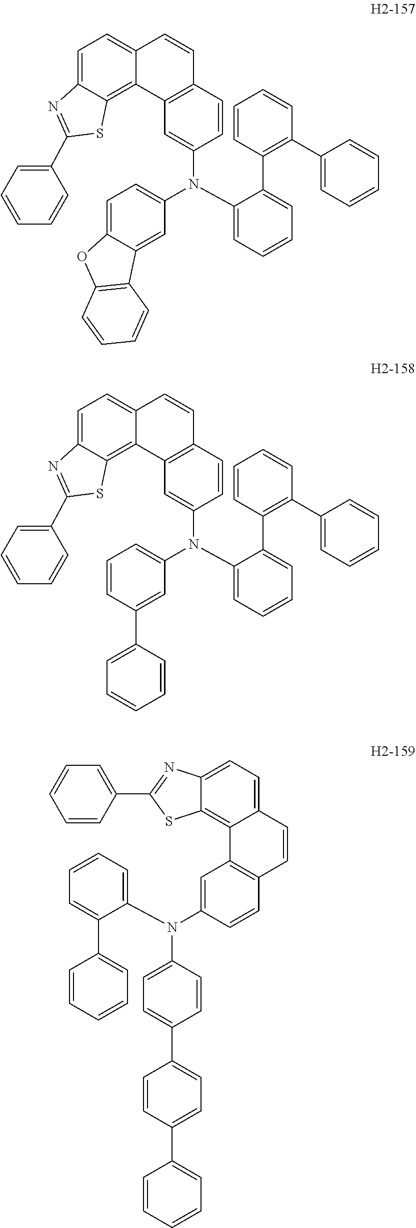 [SMILES: c1ccc(-c2nc3ccc4ccc5ccc(N(c6ccc7oc8ccccc8c7c6)c6ccccc6-c6ccccc6-c6ccccc6)cc5c4c3s2)cc1 ; c1ccc(-c2cccc(N(c3ccc4ccc5ccc6nc(-c7ccccc7)sc6c5c4c3)c3ccccc3-c3ccccc3-c3ccccc3)c2)cc1 ; c1ccc(-c2ccc(-c3ccc(N(c4ccc5ccc6ccc7nc(-c8ccccc8)sc7c6c5c4)c4ccccc4-c4ccccc4)cc3)cc2)cc1]

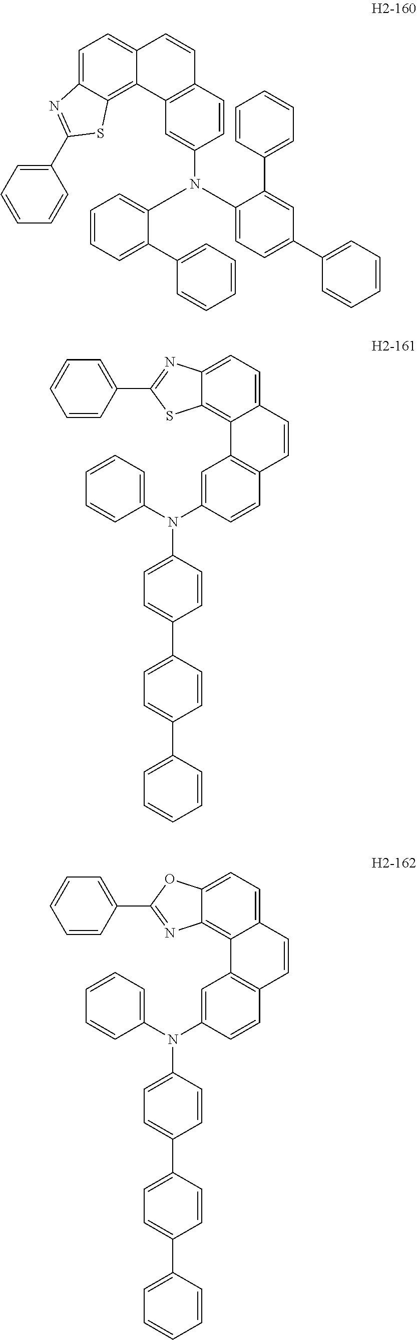 [SMILES: c1ccc(-c2ccc(N(c3ccc4ccc5ccc6nc(-c7ccccc7)sc6c5c4c3)c3ccccc3-c3ccccc3)c(-c3ccccc3)c2)cc1 ; c1ccc(-c2ccc(-c3ccc(N(c4ccccc4)c4ccc5ccc6ccc7nc(-c8ccccc8)sc7c6c5c4)cc3)cc2)cc1 ; c1ccc(-c2ccc(-c3ccc(N(c4ccccc4)c4ccc5ccc6ccc7oc(-c8ccccc8)nc7c6c5c4)cc3)cc2)cc1]

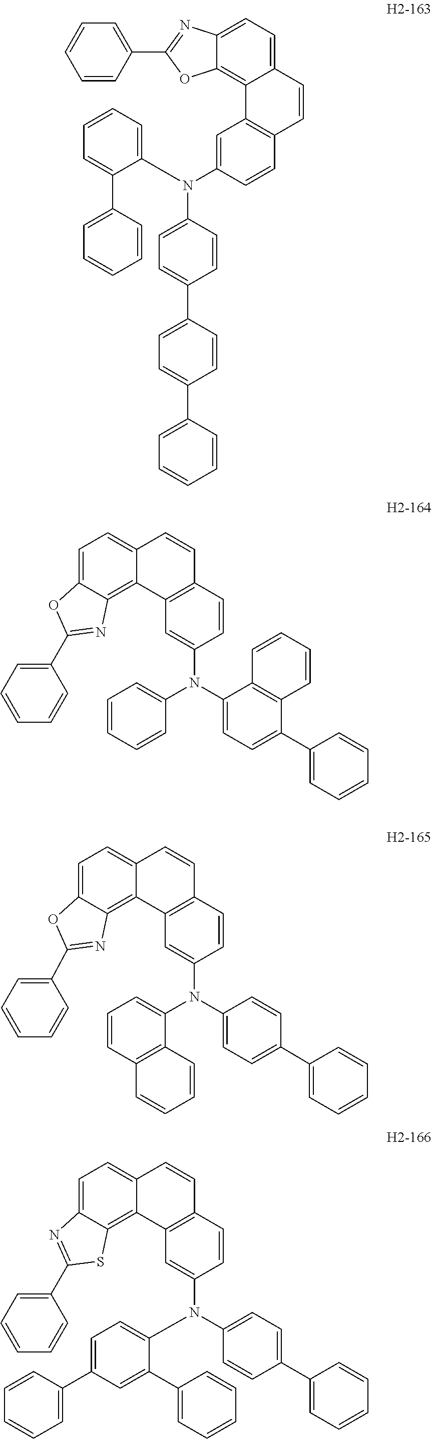 [SMILES: c1ccc(-c2ccc(-c3ccc(N(c4ccc5ccc6ccc7nc(-c8ccccc8)oc7c6c5c4)c4ccccc4-c4ccccc4)cc3)cc2)cc1 ; c1ccc(-c2nc3c(ccc4ccc5ccc(N(c6ccccc6)c6ccc(-c7ccccc7)c7ccccc67)cc5c43)o2)cc1 ; c1ccc(-c2ccc(N(c3ccc4ccc5ccc6oc(-c7ccccc7)nc6c5c4c3)c3cccc4ccccc34)cc2)cc1 ; c1ccc(-c2ccc(N(c3ccc4ccc5ccc6nc(-c7ccccc7)sc6c5c4c3)c3ccc(-c4ccccc4)cc3-c3ccccc3)cc2)cc1]

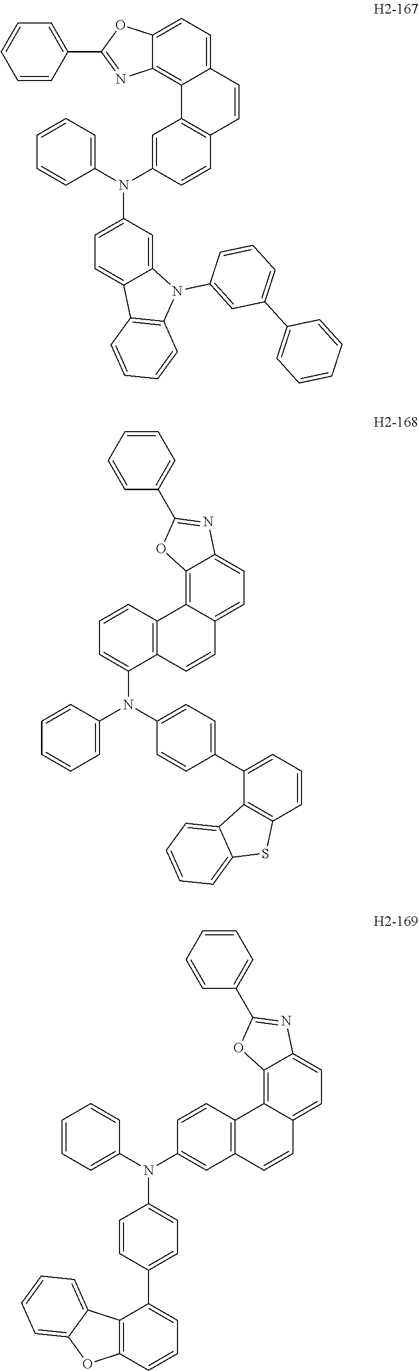 [SMILES: c1ccc(-c2cccc(-n3c4ccccc4c4ccc(N(c5ccccc5)c5ccc6ccc7ccc8oc(-c9ccccc9)nc8c7c6c5)cc43)c2)cc1 ; c1ccc(-c2nc3ccc4ccc5c(N(c6ccccc6)c6ccc(-c7cccc8sc9ccccc9c78)cc6)cccc5c4c3o2)cc1 ; c1ccc(-c2nc3ccc4ccc5cc(N(c6ccccc6)c6ccc(-c7cccc8oc9ccccc9c78)cc6)ccc5c4c3o2)cc1]

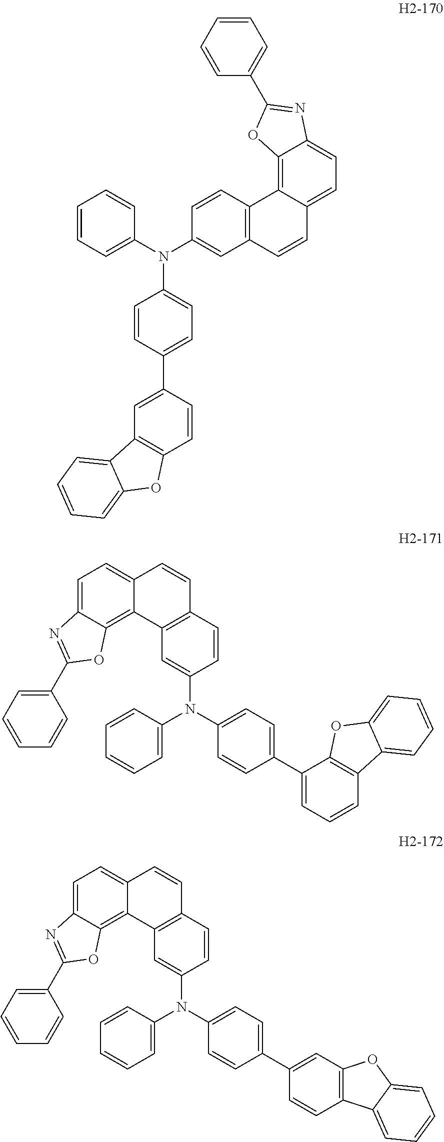 [SMILES: c1ccc(-c2nc3ccc4ccc5cc(N(c6ccccc6)c6ccc(-c7ccc8oc9ccccc9c8c7)cc6)ccc5c4c3o2)cc1 ; c1ccc(-c2nc3ccc4ccc5ccc(N(c6ccccc6)c6ccc(-c7cccc8c7oc7ccccc78)cc6)cc5c4c3o2)cc1 ; c1ccc(-c2nc3ccc4ccc5ccc(N(c6ccccc6)c6ccc(-c7ccc8c(c7)oc7ccccc78)cc6)cc5c4c3o2)cc1]

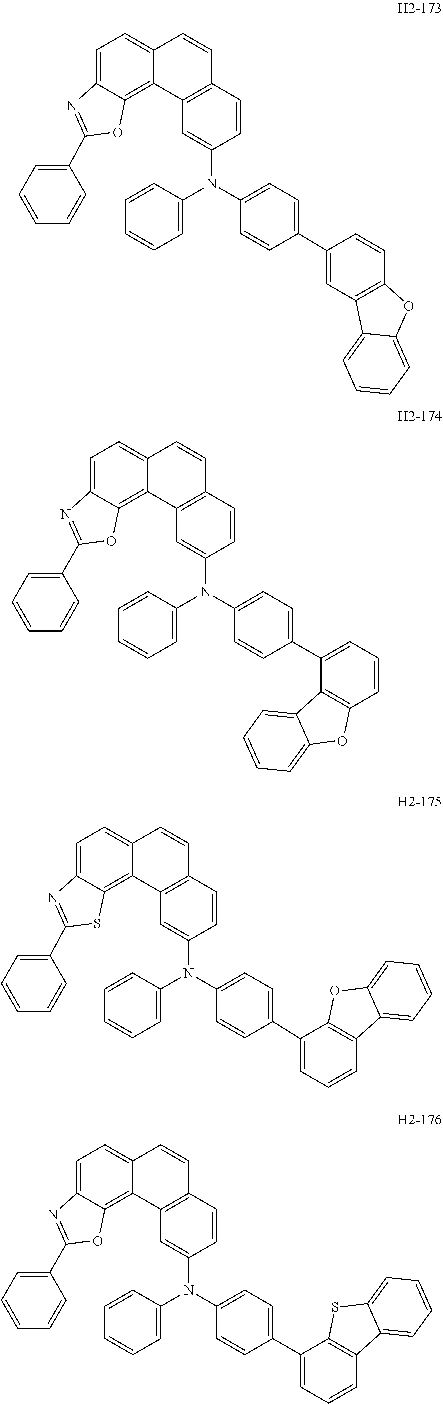 [SMILES: c1ccc(-c2nc3ccc4ccc5ccc(N(c6ccccc6)c6ccc(-c7ccc8oc9ccccc9c8c7)cc6)cc5c4c3o2)cc1 ; c1ccc(-c2nc3ccc4ccc5ccc(N(c6ccccc6)c6ccc(-c7cccc8oc9ccccc9c78)cc6)cc5c4c3o2)cc1 ; c1ccc(-c2nc3ccc4ccc5ccc(N(c6ccccc6)c6ccc(-c7cccc8c7oc7ccccc78)cc6)cc5c4c3s2)cc1 ; c1ccc(-c2nc3ccc4ccc5ccc(N(c6ccccc6)c6ccc(-c7cccc8c7sc7ccccc78)cc6)cc5c4c3o2)cc1]

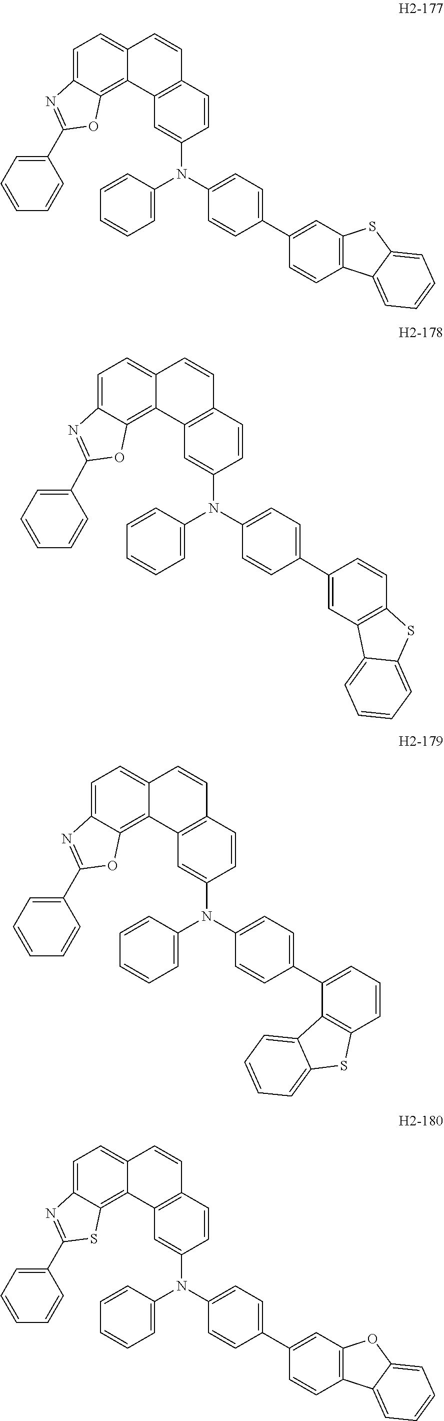 [SMILES: c1ccc(-c2nc3ccc4ccc5ccc(N(c6ccccc6)c6ccc(-c7ccc8c(c7)sc7ccccc78)cc6)cc5c4c3o2)cc1 ; c1ccc(-c2nc3ccc4ccc5ccc(N(c6ccccc6)c6ccc(-c7ccc8sc9ccccc9c8c7)cc6)cc5c4c3o2)cc1 ; c1ccc(-c2nc3ccc4ccc5ccc(N(c6ccccc6)c6ccc(-c7cccc8sc9ccccc9c78)cc6)cc5c4c3o2)cc1 ; c1ccc(-c2nc3ccc4ccc5ccc(N(c6ccccc6)c6ccc(-c7ccc8c(c7)oc7ccccc78)cc6)cc5c4c3s2)cc1]

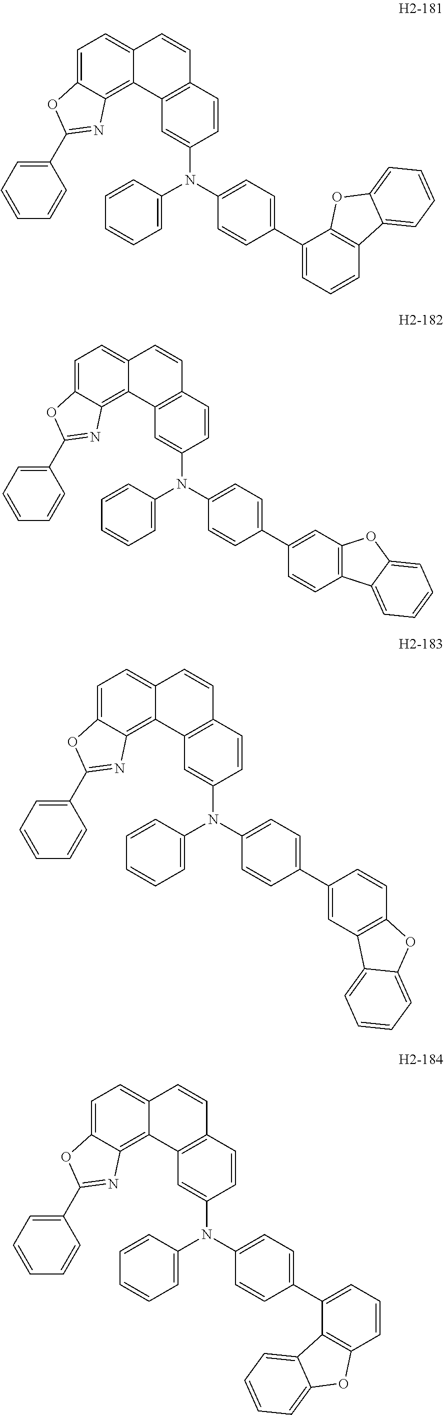 [SMILES: c1ccc(-c2nc3c(ccc4ccc5ccc(N(c6ccccc6)c6ccc(-c7cccc8c7oc7ccccc78)cc6)cc5c43)o2)cc1 ; c1ccc(-c2nc3c(ccc4ccc5ccc(N(c6ccccc6)c6ccc(-c7ccc8c(c7)oc7ccccc78)cc6)cc5c43)o2)cc1 ; c1ccc(-c2nc3c(ccc4ccc5ccc(N(c6ccccc6)c6ccc(-c7ccc8oc9ccccc9c8c7)cc6)cc5c43)o2)cc1 ; c1ccc(-c2nc3c(ccc4ccc5ccc(N(c6ccccc6)c6ccc(-c7cccc8oc9ccccc9c78)cc6)cc5c43)o2)cc1]

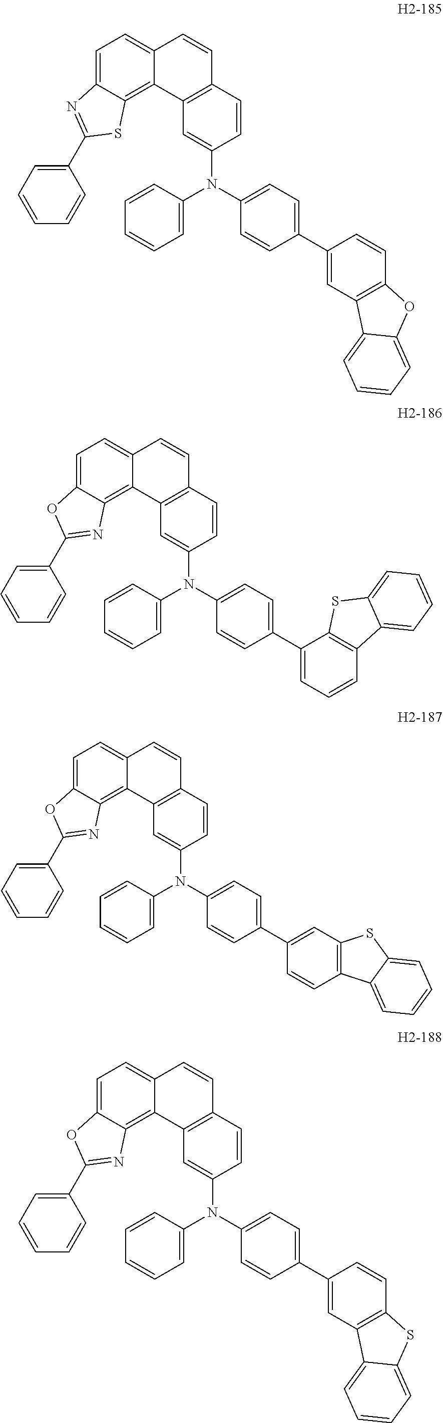 [SMILES: c1ccc(-c2nc3ccc4ccc5ccc(N(c6ccccc6)c6ccc(-c7ccc8oc9ccccc9c8c7)cc6)cc5c4c3s2)cc1 ; c1ccc(-c2nc3c(ccc4ccc5ccc(N(c6ccccc6)c6ccc(-c7cccc8c7sc7ccccc78)cc6)cc5c43)o2)cc1 ; c1ccc(-c2nc3c(ccc4ccc5ccc(N(c6ccccc6)c6ccc(-c7ccc8c(c7)sc7ccccc78)cc6)cc5c43)o2)cc1 ; c1ccc(-c2nc3c(ccc4ccc5ccc(N(c6ccccc6)c6ccc(-c7ccc8sc9ccccc9c8c7)cc6)cc5c43)o2)cc1]

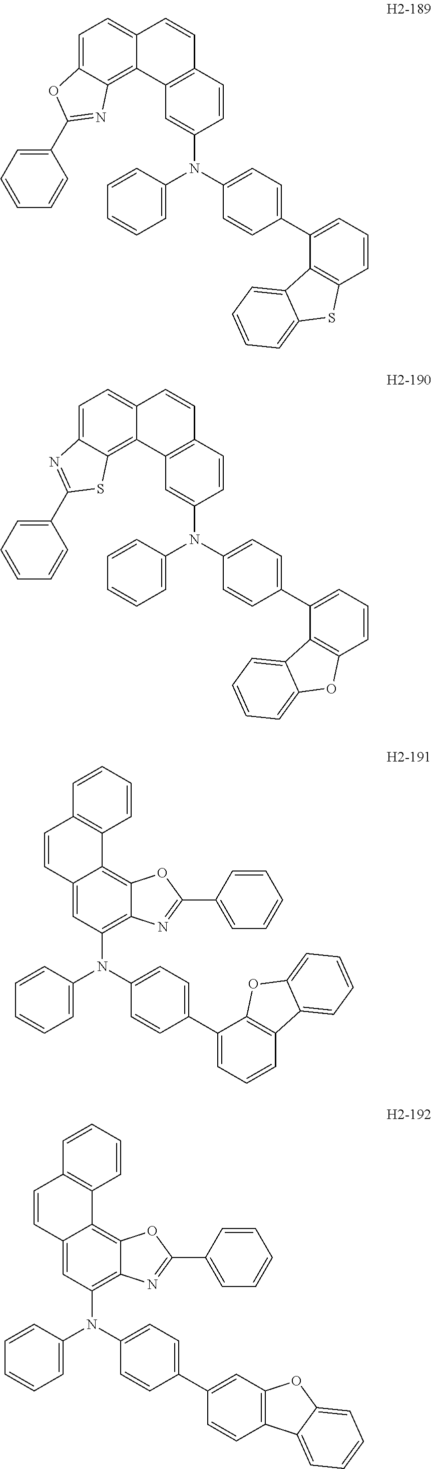 [SMILES: c1ccc(-c2nc3c(ccc4ccc5ccc(N(c6ccccc6)c6ccc(-c7cccc8sc9ccccc9c78)cc6)cc5c43)o2)cc1 ; c1ccc(-c2nc3ccc4ccc5ccc(N(c6ccccc6)c6ccc(-c7cccc8oc9ccccc9c78)cc6)cc5c4c3s2)cc1 ; c1ccc(-c2nc3c(N(c4ccccc4)c4ccc(-c5cccc6c5oc5ccccc56)cc4)cc4ccc5ccccc5c4c3o2)cc1 ; c1ccc(-c2nc3c(N(c4ccccc4)c4ccc(-c5ccc6c(c5)oc5ccccc56)cc4)cc4ccc5ccccc5c4c3o2)cc1]

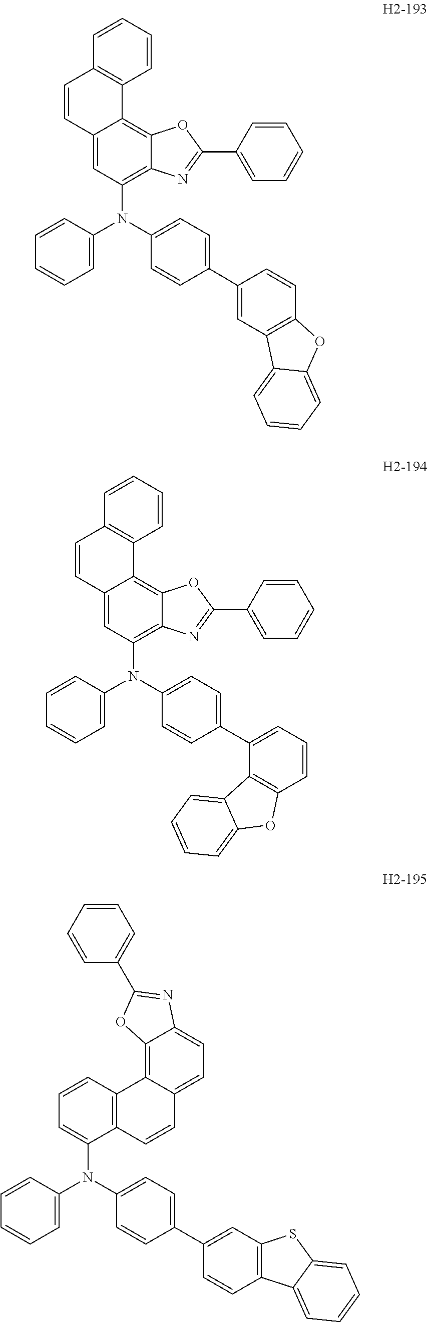 [SMILES: c1ccc(-c2nc3c(N(c4ccccc4)c4ccc(-c5ccc6oc7ccccc7c6c5)cc4)cc4ccc5ccccc5c4c3o2)cc1 ; c1ccc(-c2nc3c(N(c4ccccc4)c4ccc(-c5cccc6oc7ccccc7c56)cc4)cc4ccc5ccccc5c4c3o2)cc1 ; c1ccc(-c2nc3ccc4ccc5c(N(c6ccccc6)c6ccc(-c7ccc8c(c7)sc7ccccc78)cc6)cccc5c4c3o2)cc1]

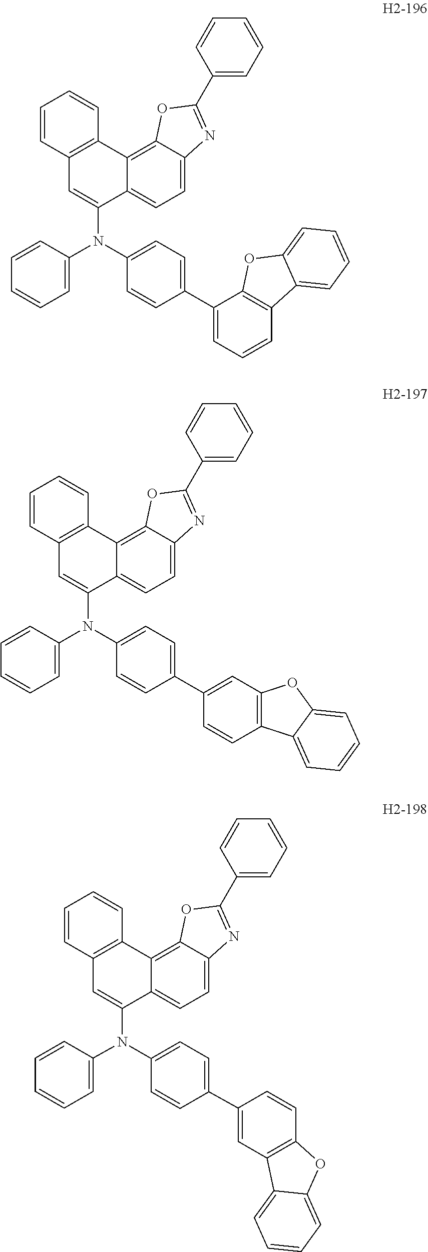

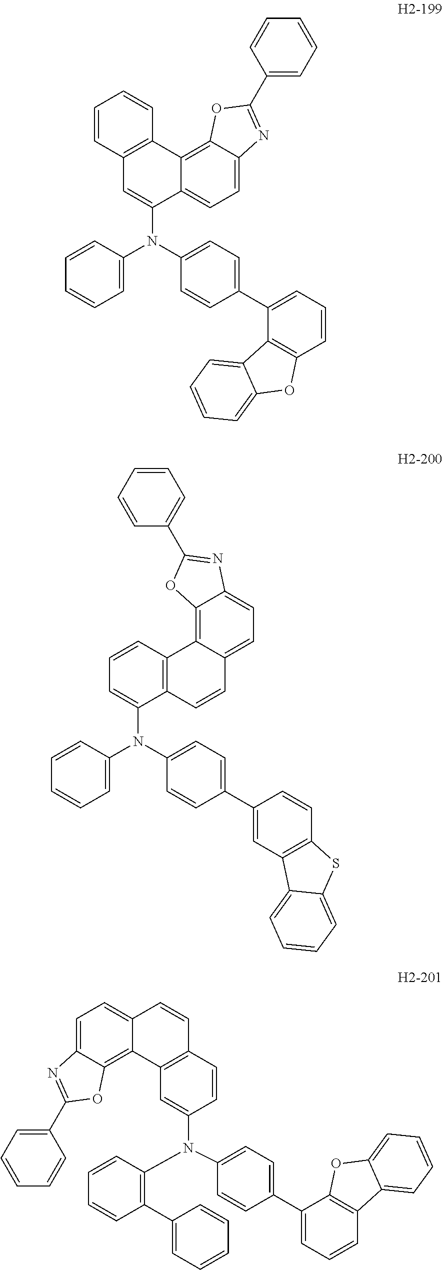 [SMILES: c1ccc(-c2nc3ccc4c(N(c5ccccc5)c5ccc(-c6cccc7oc8ccccc8c67)cc5)cc5ccccc5c4c3o2)cc1 ; c1ccc(-c2nc3ccc4ccc5c(N(c6ccccc6)c6ccc(-c7ccc8sc9ccccc9c8c7)cc6)cccc5c4c3o2)cc1 ; c1ccc(-c2nc3ccc4ccc5ccc(N(c6ccc(-c7cccc8c7oc7ccccc78)cc6)c6ccccc6-c6ccccc6)cc5c4c3o2)cc1]

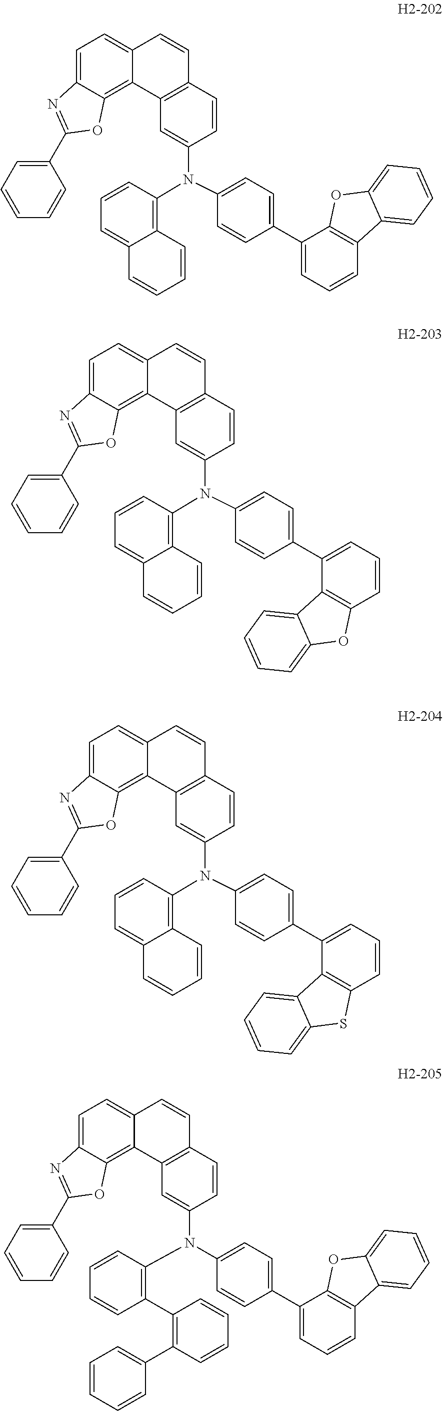 [SMILES: c1ccc(-c2nc3ccc4ccc5ccc(N(c6ccc(-c7cccc8c7oc7ccccc78)cc6)c6cccc7ccccc67)cc5c4c3o2)cc1 ; c1ccc(-c2nc3ccc4ccc5ccc(N(c6ccc(-c7cccc8oc9ccccc9c78)cc6)c6cccc7ccccc67)cc5c4c3o2)cc1 ; c1ccc(-c2nc3ccc4ccc5ccc(N(c6ccc(-c7cccc8sc9ccccc9c78)cc6)c6cccc7ccccc67)cc5c4c3o2)cc1 ; c1ccc(-c2nc3ccc4ccc5ccc(N(c6ccc(-c7cccc8c7oc7ccccc78)cc6)c6ccccc6-c6ccccc6-c6ccccc6)cc5c4c3o2)cc1]

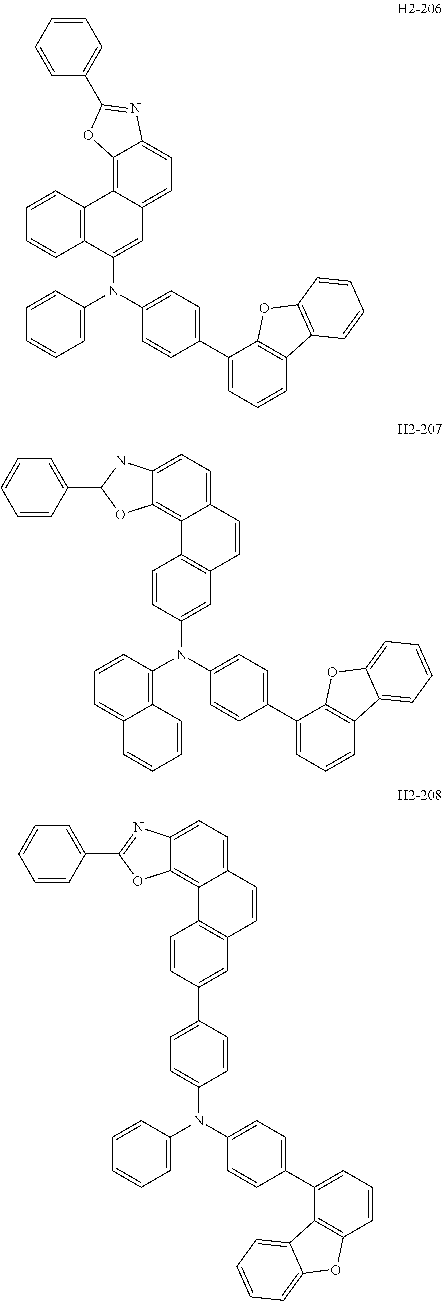

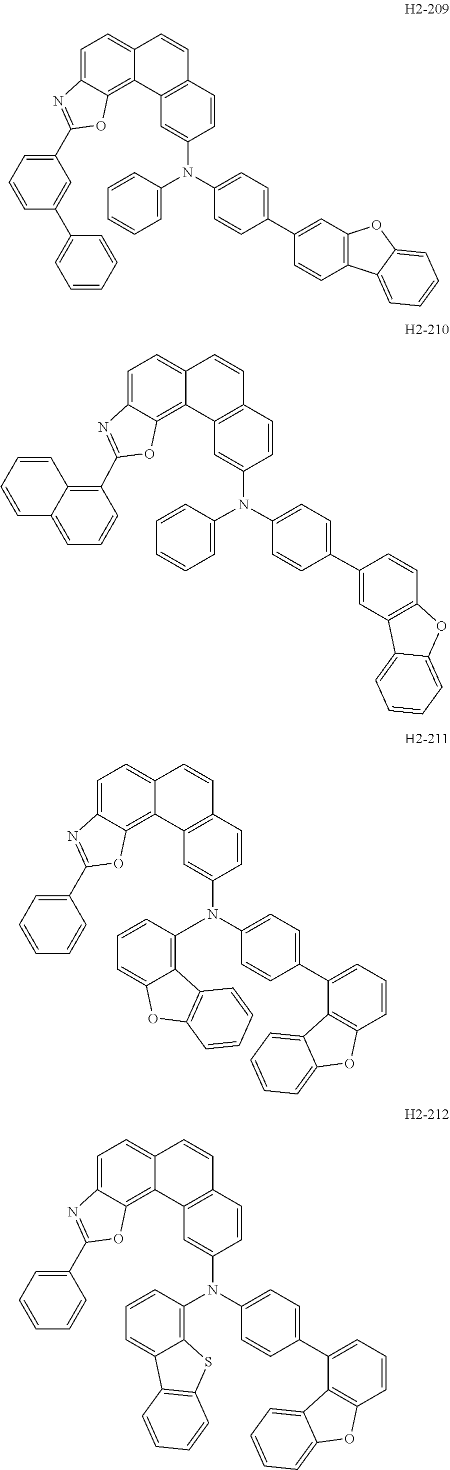 [SMILES: c1ccc(-c2cccc(-c3nc4ccc5ccc6ccc(N(c7ccccc7)c7ccc(-c8ccc9c(c8)oc8ccccc89)cc7)cc6c5c4o3)c2)cc1 ; c1ccc(N(c2ccc(-c3ccc4oc5ccccc5c4c3)cc2)c2ccc3ccc4ccc5nc(-c6cccc7ccccc67)oc5c4c3c2)cc1 ; c1ccc(-c2nc3ccc4ccc5ccc(N(c6ccc(-c7cccc8oc9ccccc9c78)cc6)c6cccc7oc8ccccc8c67)cc5c4c3o2)cc1 ; c1ccc(-c2nc3ccc4ccc5ccc(N(c6ccc(-c7cccc8oc9ccccc9c78)cc6)c6cccc7c6sc6ccccc67)cc5c4c3o2)cc1]

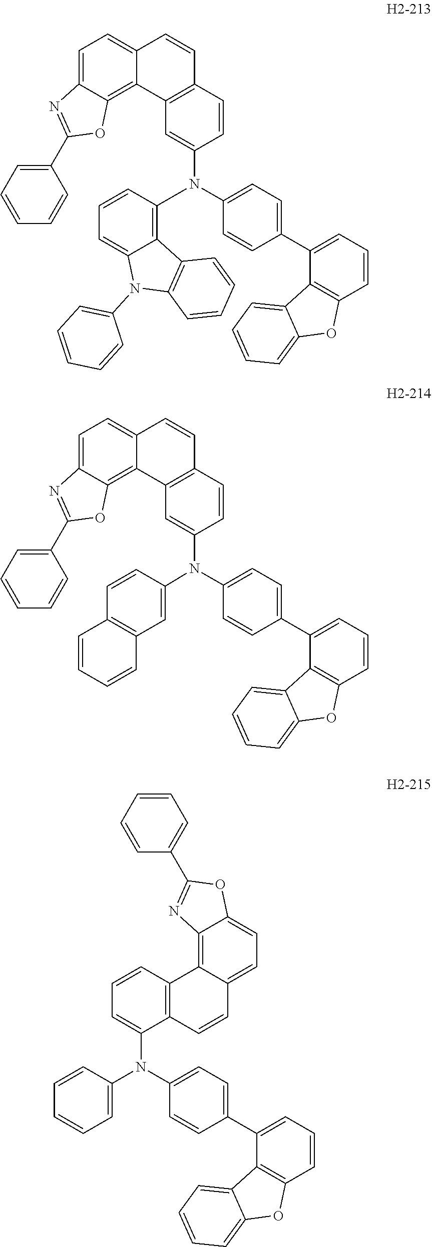 [SMILES: c1ccc(-c2nc3ccc4ccc5ccc(N(c6ccc(-c7cccc8oc9ccccc9c78)cc6)c6cccc7c6c6ccccc6n7-c6ccccc6)cc5c4c3o2)cc1 ; c1ccc(-c2nc3ccc4ccc5ccc(N(c6ccc(-c7cccc8oc9ccccc9c78)cc6)c6ccc7ccccc7c6)cc5c4c3o2)cc1 ; c1ccc(-c2nc3c(ccc4ccc5c(N(c6ccccc6)c6ccc(-c7cccc8oc9ccccc9c78)cc6)cccc5c43)o2)cc1]

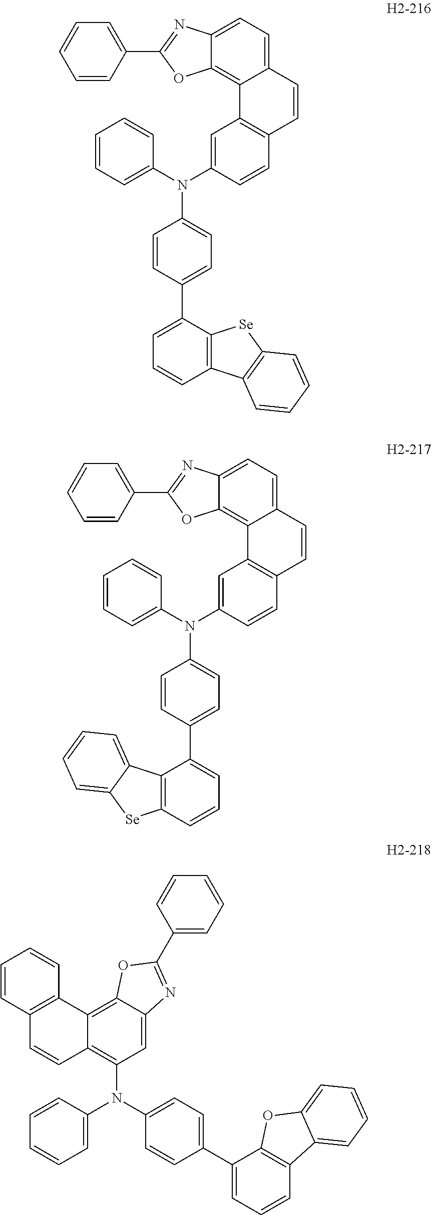 [SMILES: c1ccc(-c2nc3ccc4ccc5ccc(N(c6ccccc6)c6ccc(-c7cccc8c7[se]c7ccccc78)cc6)cc5c4c3o2)cc1 ; c1ccc(-c2nc3ccc4ccc5ccc(N(c6ccccc6)c6ccc(-c7cccc8[se]c9ccccc9c78)cc6)cc5c4c3o2)cc1 ; c1ccc(-c2nc3cc(N(c4ccccc4)c4ccc(-c5cccc6c5oc5ccccc56)cc4)c4ccc5ccccc5c4c3o2)cc1]

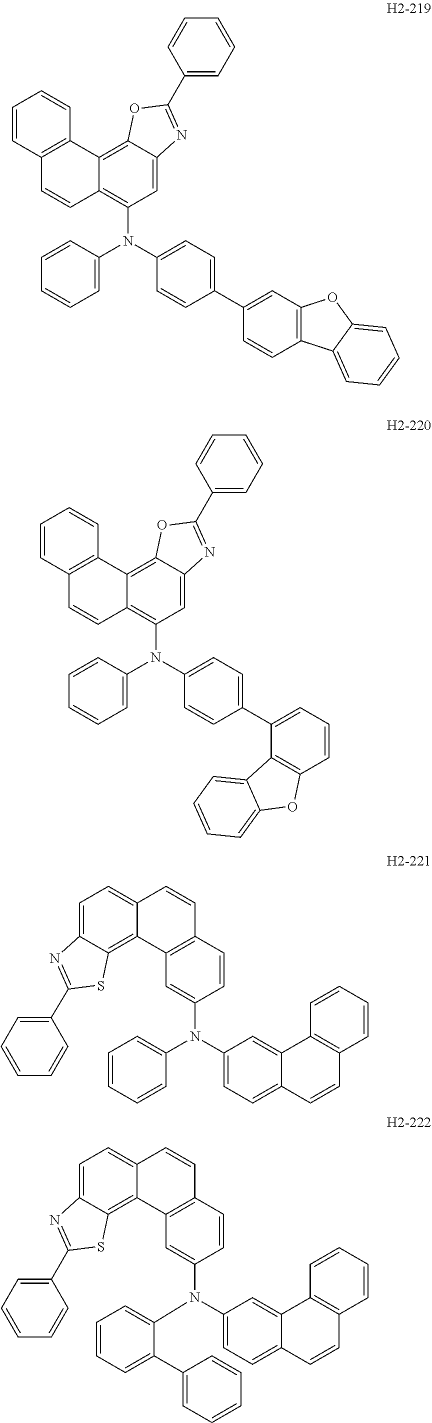 [SMILES: c1ccc(-c2nc3cc(N(c4ccccc4)c4ccc(-c5ccc6c(c5)oc5ccccc56)cc4)c4ccc5ccccc5c4c3o2)cc1 ; c1ccc(-c2nc3cc(N(c4ccccc4)c4ccc(-c5cccc6oc7ccccc7c56)cc4)c4ccc5ccccc5c4c3o2)cc1 ; c1ccc(-c2nc3ccc4ccc5ccc(N(c6ccccc6)c6ccc7ccc8ccccc8c7c6)cc5c4c3s2)cc1 ; c1ccc(-c2nc3ccc4ccc5ccc(N(c6ccc7ccc8ccccc8c7c6)c6ccccc6-c6ccccc6)cc5c4c3s2)cc1]

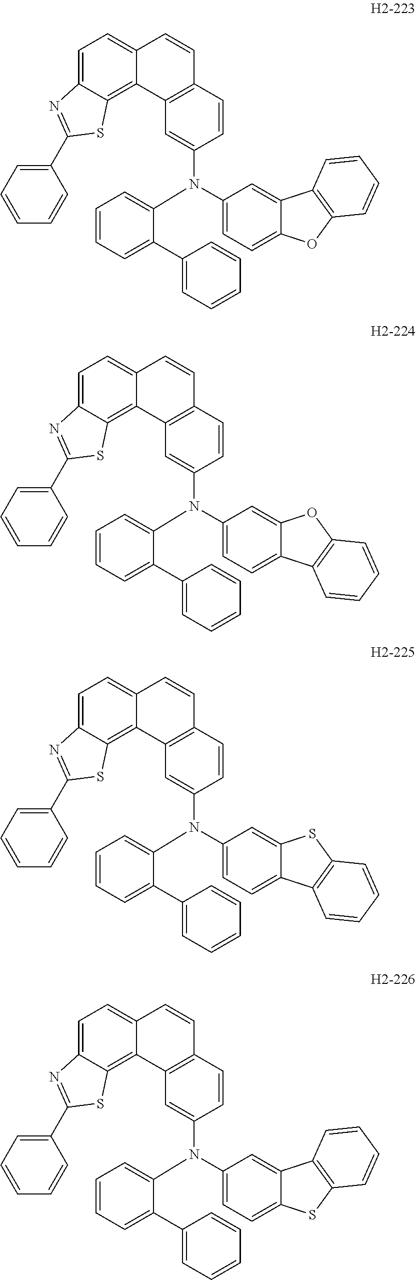 [SMILES: c1ccc(-c2nc3ccc4ccc5ccc(N(c6ccc7oc8ccccc8c7c6)c6ccccc6-c6ccccc6)cc5c4c3s2)cc1 ; c1ccc(-c2nc3ccc4ccc5ccc(N(c6ccc7c(c6)oc6ccccc67)c6ccccc6-c6ccccc6)cc5c4c3s2)cc1 ; c1ccc(-c2nc3ccc4ccc5ccc(N(c6ccc7c(c6)sc6ccccc67)c6ccccc6-c6ccccc6)cc5c4c3s2)cc1 ; c1ccc(-c2nc3ccc4ccc5ccc(N(c6ccc7sc8ccccc8c7c6)c6ccccc6-c6ccccc6)cc5c4c3s2)cc1]

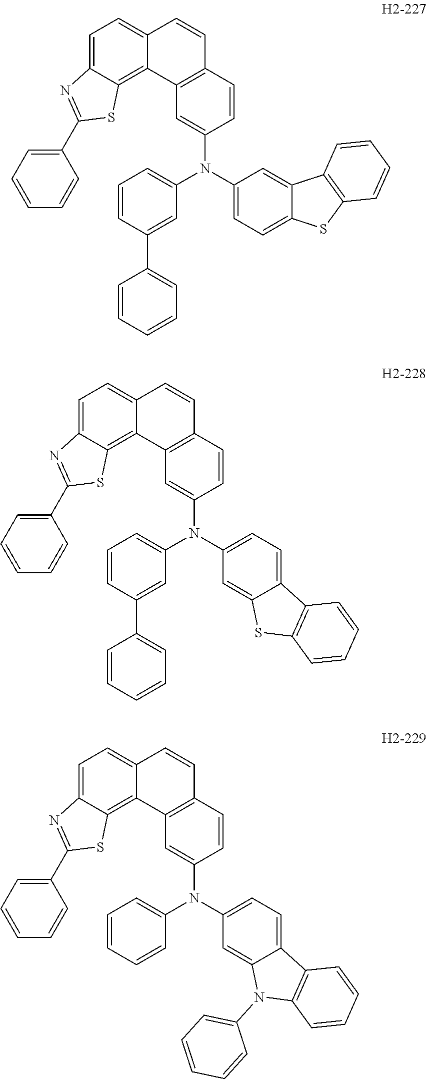 [SMILES: c1ccc(-c2cccc(N(c3ccc4sc5ccccc5c4c3)c3ccc4ccc5ccc6nc(-c7ccccc7)sc6c5c4c3)c2)cc1 ; c1ccc(-c2cccc(N(c3ccc4c(c3)sc3ccccc34)c3ccc4ccc5ccc6nc(-c7ccccc7)sc6c5c4c3)c2)cc1 ; c1ccc(-c2nc3ccc4ccc5ccc(N(c6ccccc6)c6ccc7c8ccccc8n(-c8ccccc8)c7c6)cc5c4c3s2)cc1]

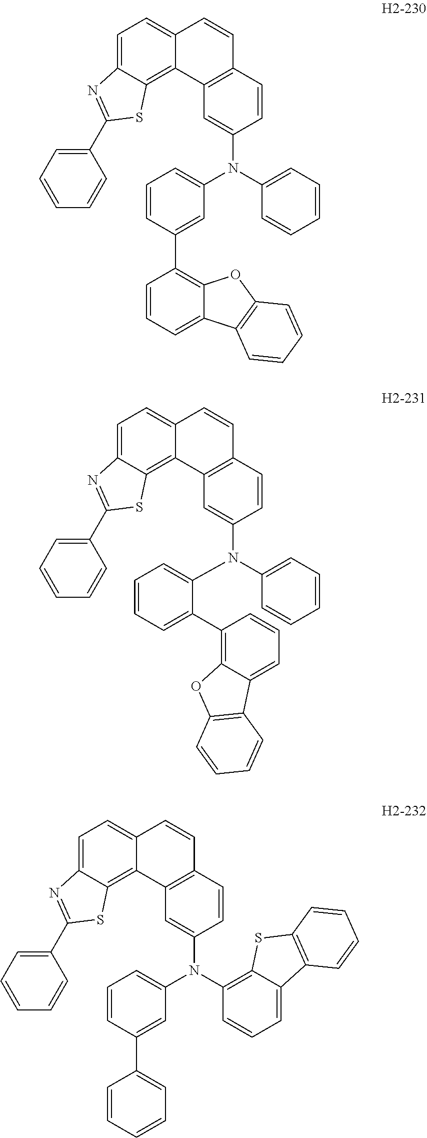 [SMILES: c1ccc(-c2nc3ccc4ccc5ccc(N(c6ccccc6)c6cccc(-c7cccc8c7oc7ccccc78)c6)cc5c4c3s2)cc1 ; c1ccc(-c2nc3ccc4ccc5ccc(N(c6ccccc6)c6ccccc6-c6cccc7c6oc6ccccc67)cc5c4c3s2)cc1 ; c1ccc(-c2cccc(N(c3ccc4ccc5ccc6nc(-c7ccccc7)sc6c5c4c3)c3cccc4c3sc3ccccc34)c2)cc1]

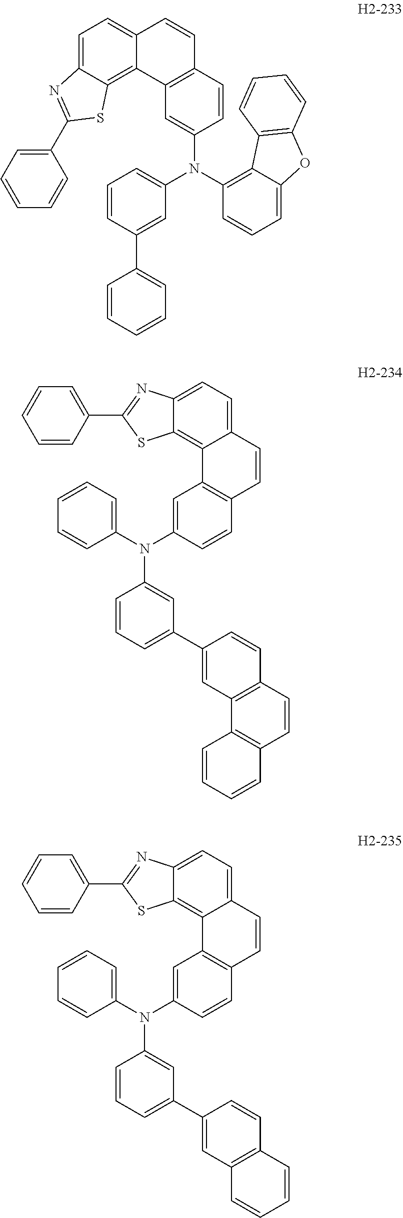 [SMILES: c1ccc(-c2cccc(N(c3ccc4ccc5ccc6nc(-c7ccccc7)sc6c5c4c3)c3cccc4oc5ccccc5c34)c2)cc1 ; c1ccc(-c2nc3ccc4ccc5ccc(N(c6ccccc6)c6cccc(-c7ccc8ccc9ccccc9c8c7)c6)cc5c4c3s2)cc1 ; c1ccc(-c2nc3ccc4ccc5ccc(N(c6ccccc6)c6cccc(-c7ccc8ccccc8c7)c6)cc5c4c3s2)cc1]

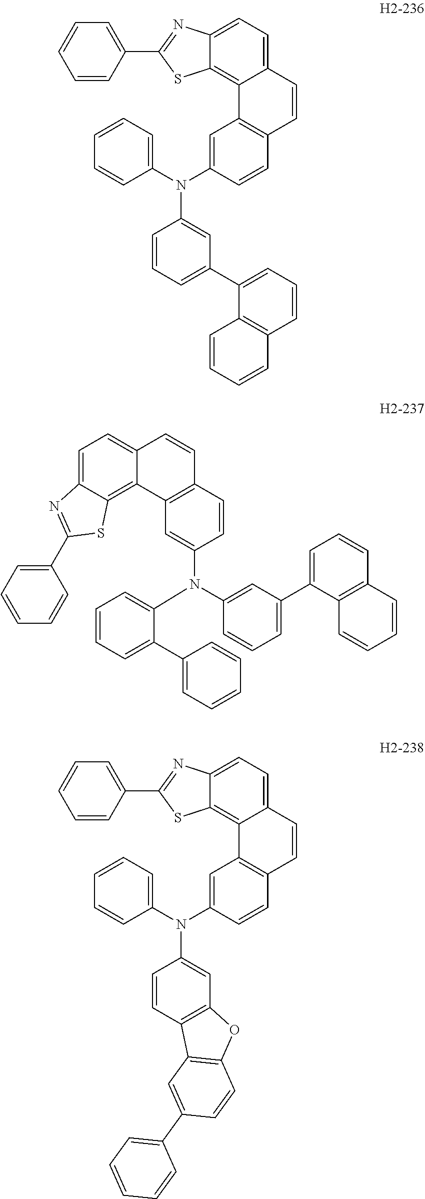 [SMILES: c1ccc(-c2nc3ccc4ccc5ccc(N(c6ccccc6)c6cccc(-c7cccc8ccccc78)c6)cc5c4c3s2)cc1 ; c1ccc(-c2nc3ccc4ccc5ccc(N(c6cccc(-c7cccc8ccccc78)c6)c6ccccc6-c6ccccc6)cc5c4c3s2)cc1 ; c1ccc(-c2ccc3oc4cc(N(c5ccccc5)c5ccc6ccc7ccc8nc(-c9ccccc9)sc8c7c6c5)ccc4c3c2)cc1]

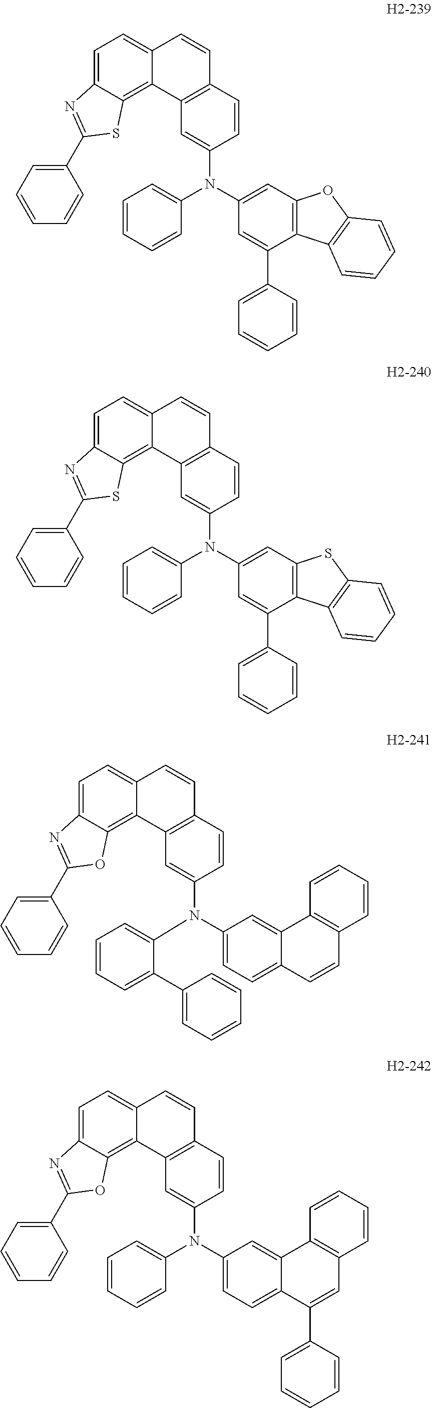 [SMILES: c1ccc(-c2nc3ccc4ccc5ccc(N(c6ccccc6)c6cc(-c7ccccc7)c7c(c6)oc6ccccc67)cc5c4c3s2)cc1 ; c1ccc(-c2nc3ccc4ccc5ccc(N(c6ccccc6)c6cc(-c7ccccc7)c7c(c6)sc6ccccc67)cc5c4c3s2)cc1 ; c1ccc(-c2nc3ccc4ccc5ccc(N(c6ccc7ccc8ccccc8c7c6)c6ccccc6-c6ccccc6)cc5c4c3o2)cc1 ; c1ccc(-c2nc3ccc4ccc5ccc(N(c6ccccc6)c6ccc7c(-c8ccccc8)cc8ccccc8c7c6)cc5c4c3o2)cc1]

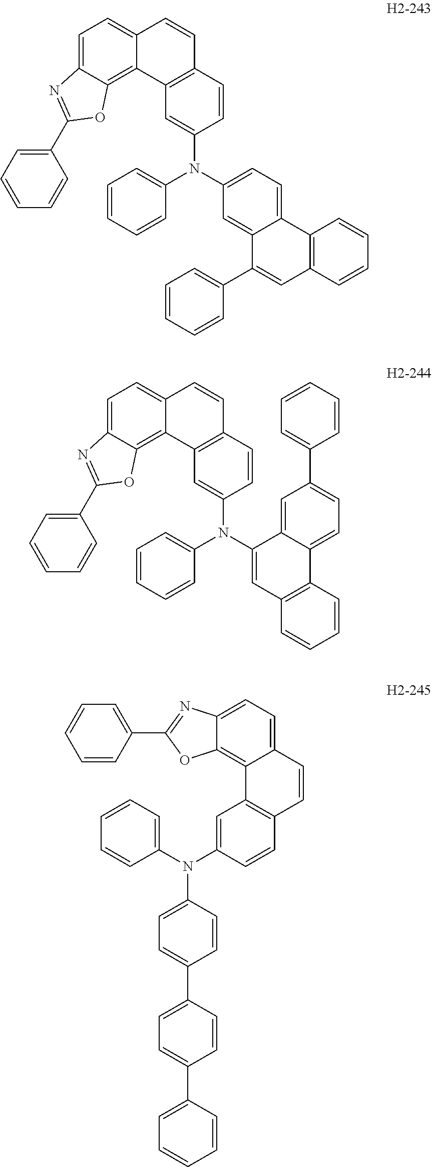 [SMILES: c1ccc(-c2nc3ccc4ccc5ccc(N(c6ccccc6)c6ccc7c(c6)c(-c6ccccc6)cc6ccccc67)cc5c4c3o2)cc1 ; c1ccc(-c2ccc3c(c2)c(N(c2ccccc2)c2ccc4ccc5ccc6nc(-c7ccccc7)oc6c5c4c2)cc2ccccc23)cc1 ; c1ccc(-c2ccc(-c3ccc(N(c4ccccc4)c4ccc5ccc6ccc7nc(-c8ccccc8)oc7c6c5c4)cc3)cc2)cc1]

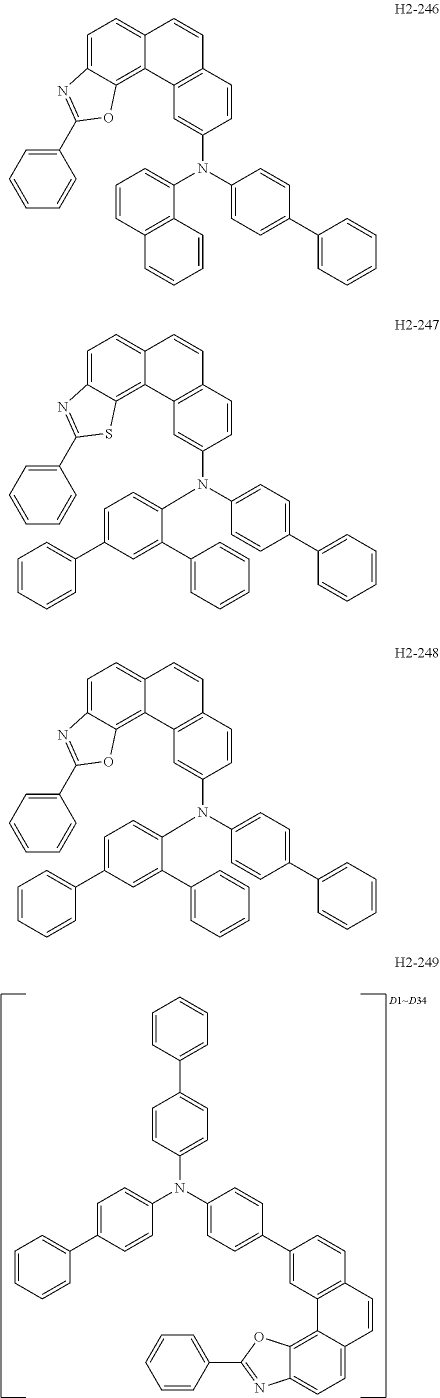

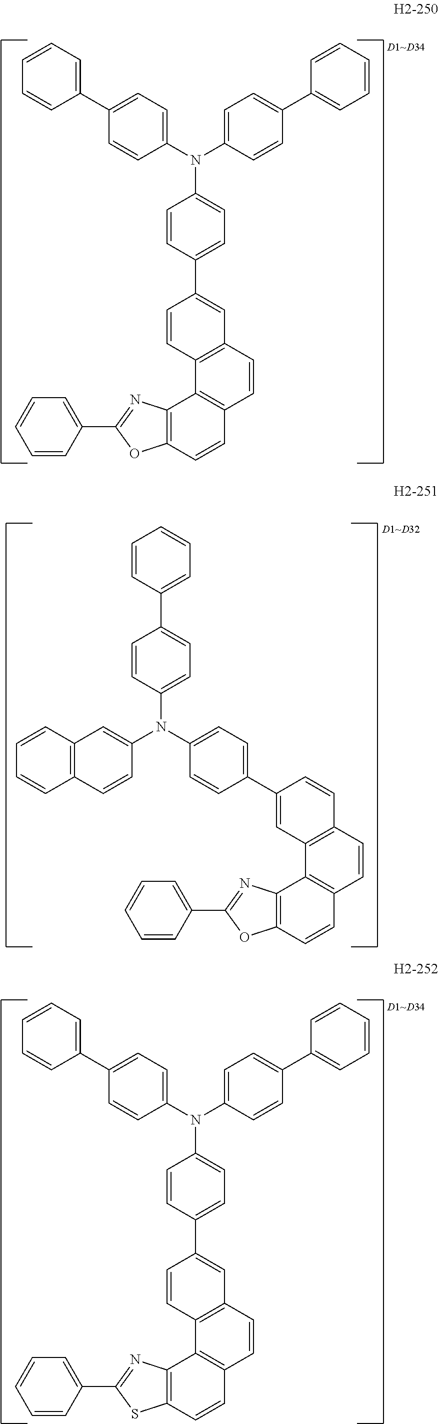

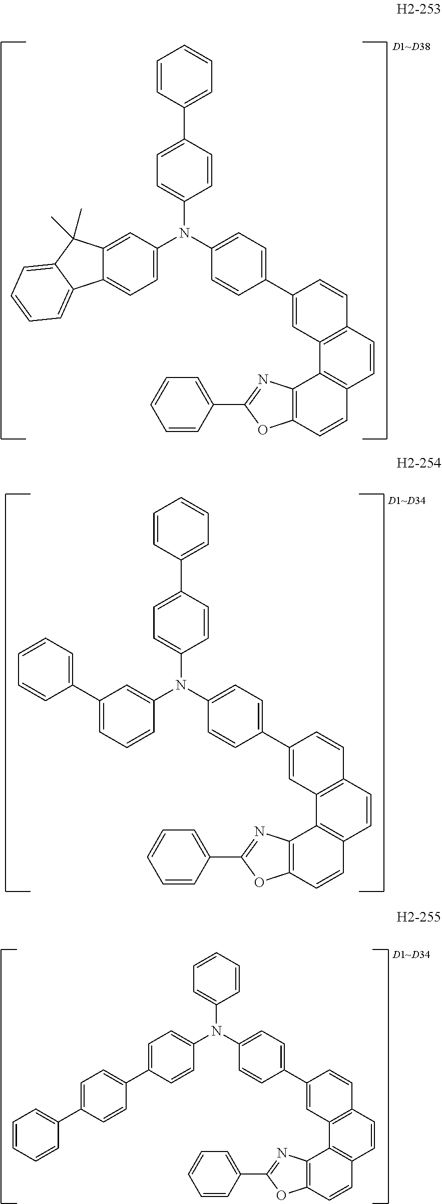

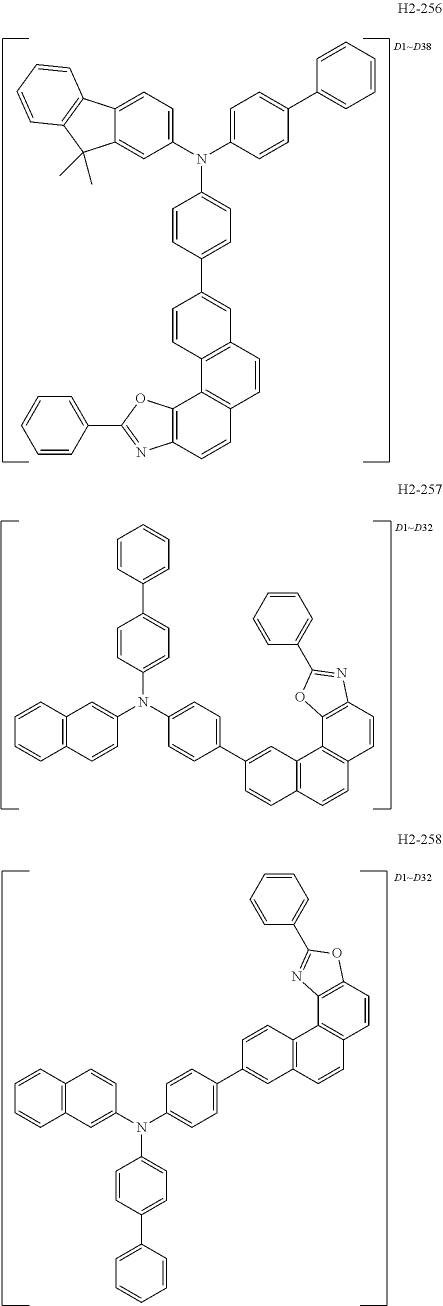

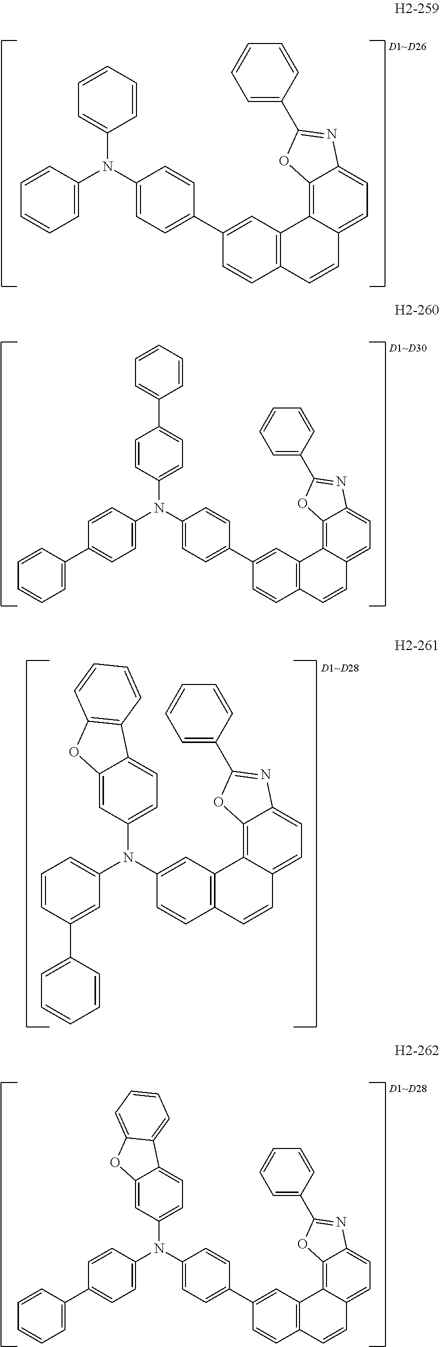

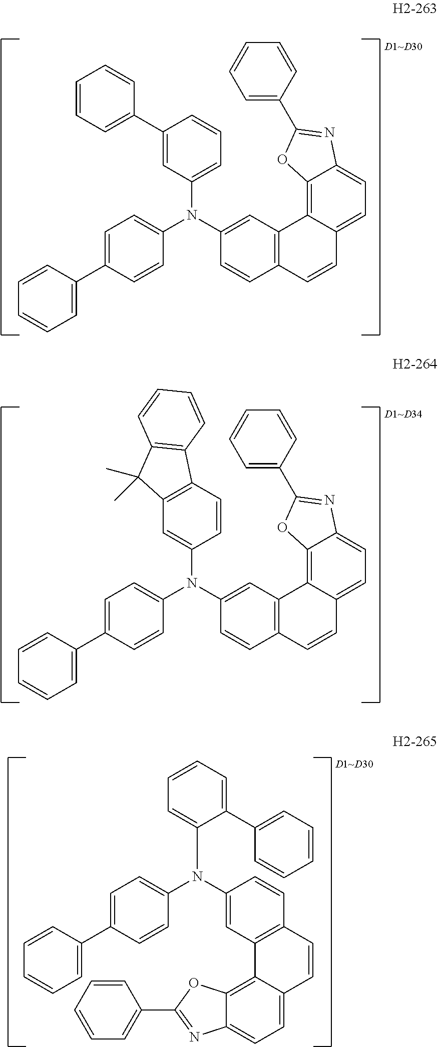

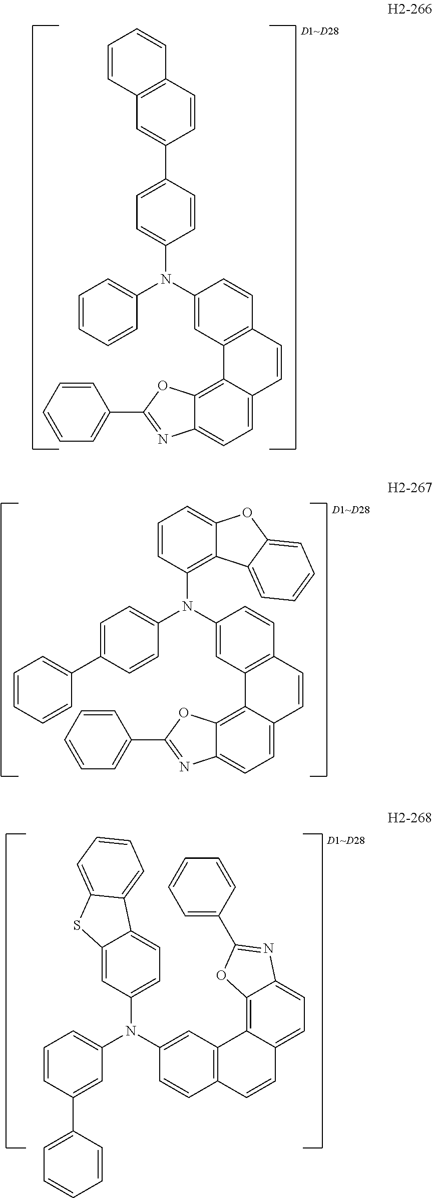

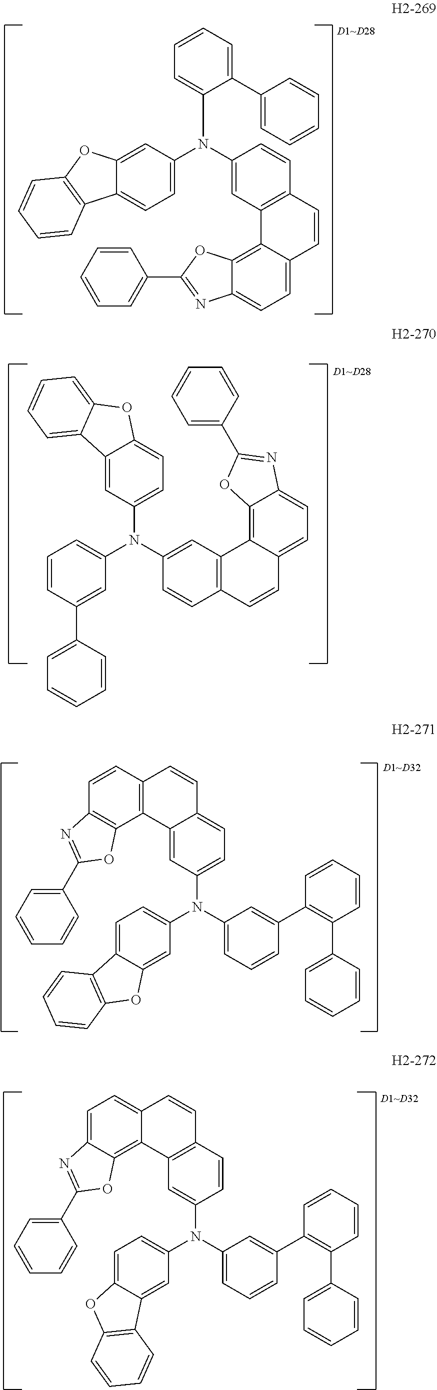

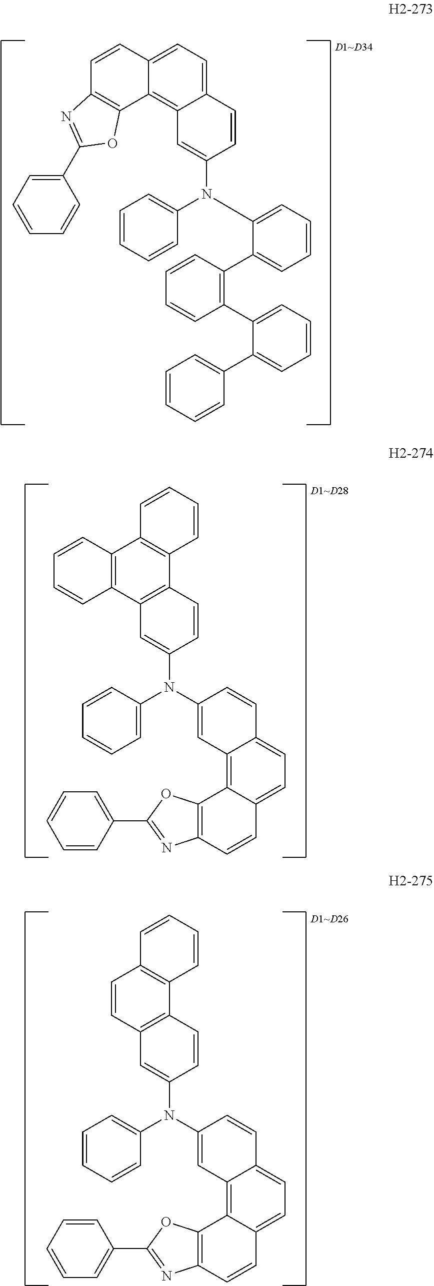

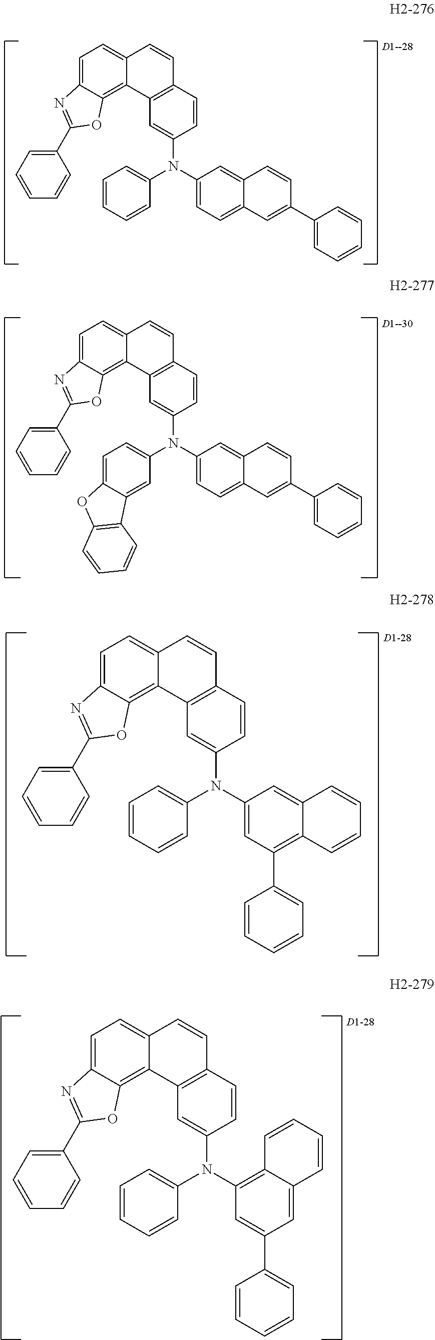

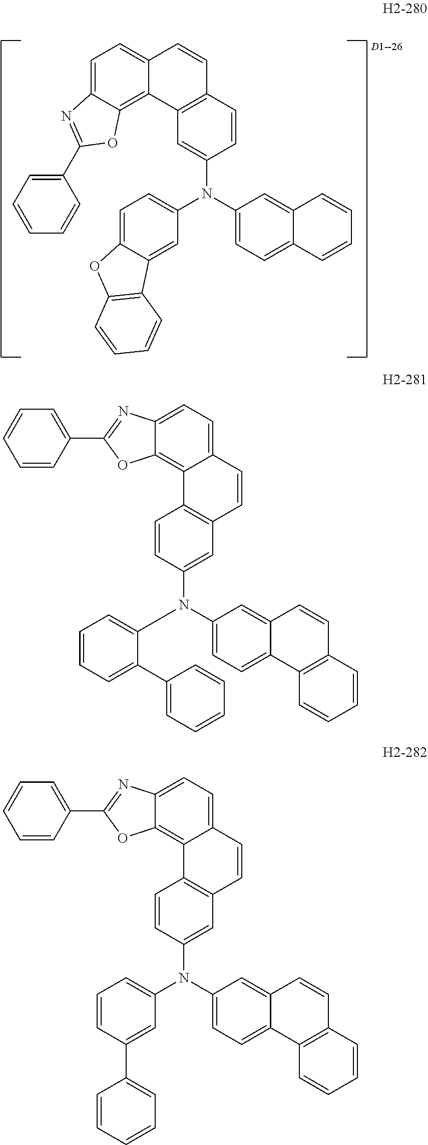

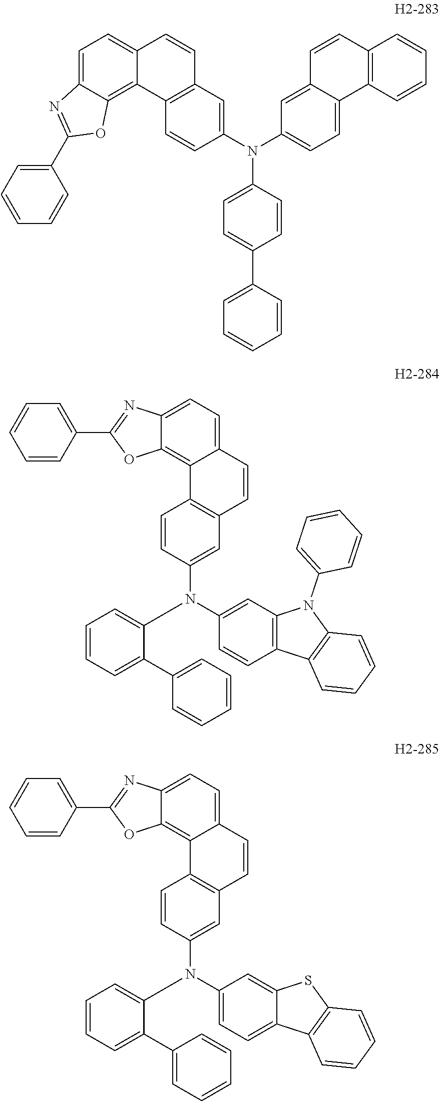 [SMILES: c1ccc(-c2ccc(N(c3ccc4c(ccc5ccccc54)c3)c3ccc4c(ccc5ccc6nc(-c7ccccc7)oc6c54)c3)cc2)cc1 ; c1ccc(-c2nc3ccc4ccc5cc(N(c6ccc7c8ccccc8n(-c8ccccc8)c7c6)c6ccccc6-c6ccccc6)ccc5c4c3o2)cc1 ; c1ccc(-c2nc3ccc4ccc5cc(N(c6ccc7c(c6)sc6ccccc67)c6ccccc6-c6ccccc6)ccc5c4c3o2)cc1]

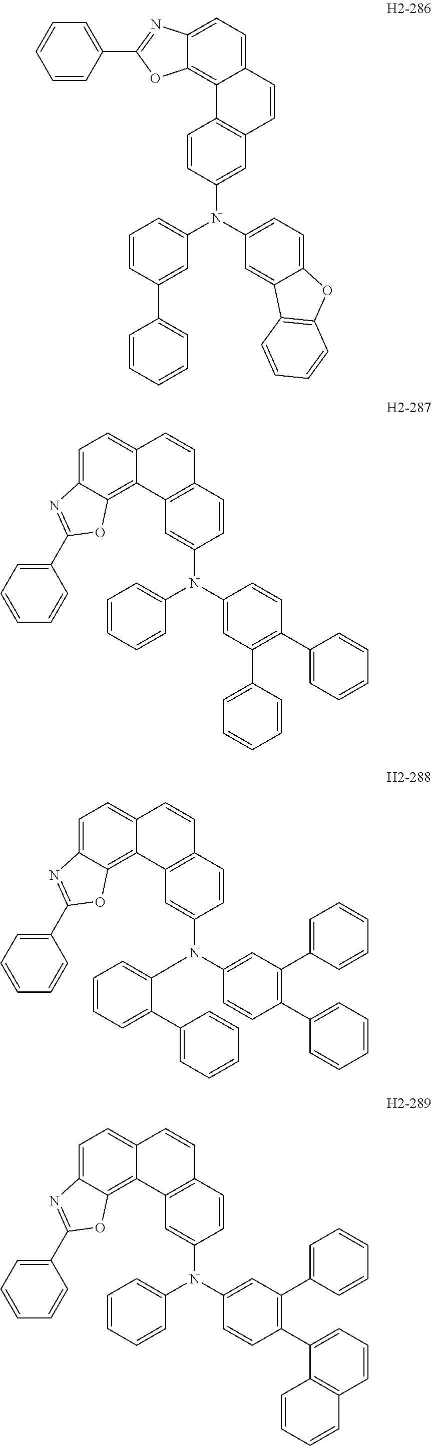 [SMILES: c1ccc(-c2cccc(N(c3ccc4c(ccc5ccc6nc(-c7ccccc7)oc6c54)c3)c3ccc4oc5ccccc5c4c3)c2)cc1 ; c1ccc(-c2nc3ccc4ccc5ccc(N(c6ccccc6)c6ccc(-c7ccccc7)c(-c7ccccc7)c6)cc5c4c3o2)cc1 ; c1ccc(-c2nc3ccc4ccc5ccc(N(c6ccc(-c7ccccc7)c(-c7ccccc7)c6)c6ccccc6-c6ccccc6)cc5c4c3o2)cc1 ; c1ccc(-c2nc3ccc4ccc5ccc(N(c6ccccc6)c6ccc(-c7cccc8ccccc78)c(-c7ccccc7)c6)cc5c4c3o2)cc1]

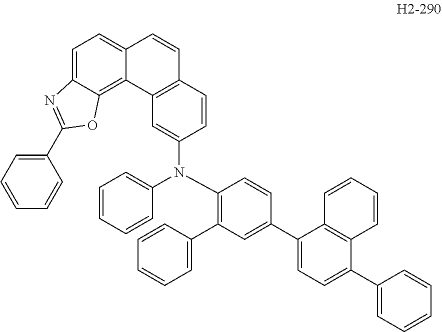 [SMILES: c1ccc(-c2nc3ccc4ccc5ccc(N(c6ccccc6)c6ccc(-c7ccc(-c8ccccc8)c8ccccc78)cc6-c6ccccc6)cc5c4c3o2)cc1]

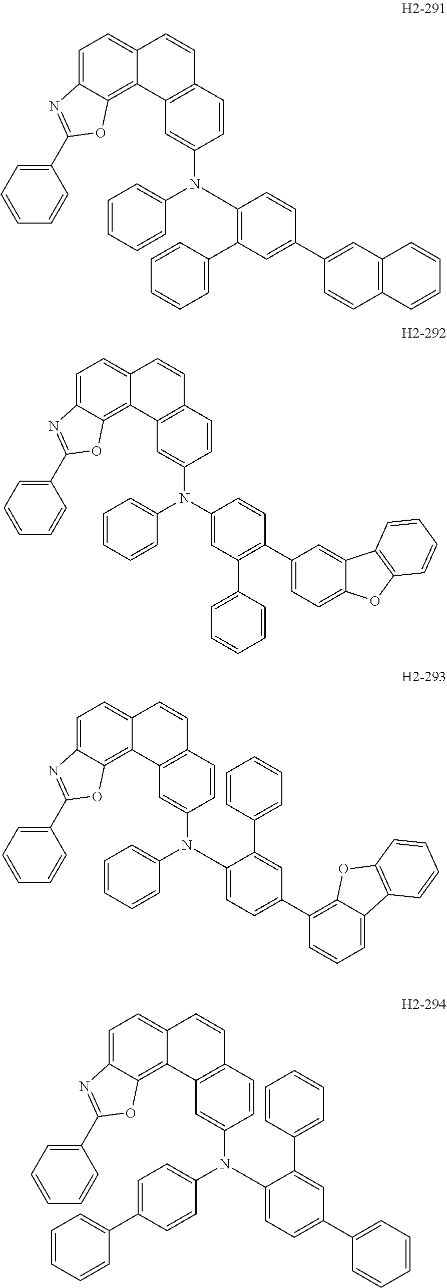 [SMILES: c1ccc(-c2nc3ccc4ccc5ccc(N(c6ccccc6)c6ccc(-c7ccc8ccccc8c7)cc6-c6ccccc6)cc5c4c3o2)cc1 ; c1ccc(-c2nc3ccc4ccc5ccc(N(c6ccccc6)c6ccc(-c7ccc8oc9ccccc9c8c7)c(-c7ccccc7)c6)cc5c4c3o2)cc1 ; c1ccc(-c2nc3ccc4ccc5ccc(N(c6ccccc6)c6ccc(-c7cccc8c7oc7ccccc78)cc6-c6ccccc6)cc5c4c3o2)cc1 ; c1ccc(-c2ccc(N(c3ccc4ccc5ccc6nc(-c7ccccc7)oc6c5c4c3)c3ccc(-c4ccccc4)cc3-c3ccccc3)cc2)cc1]

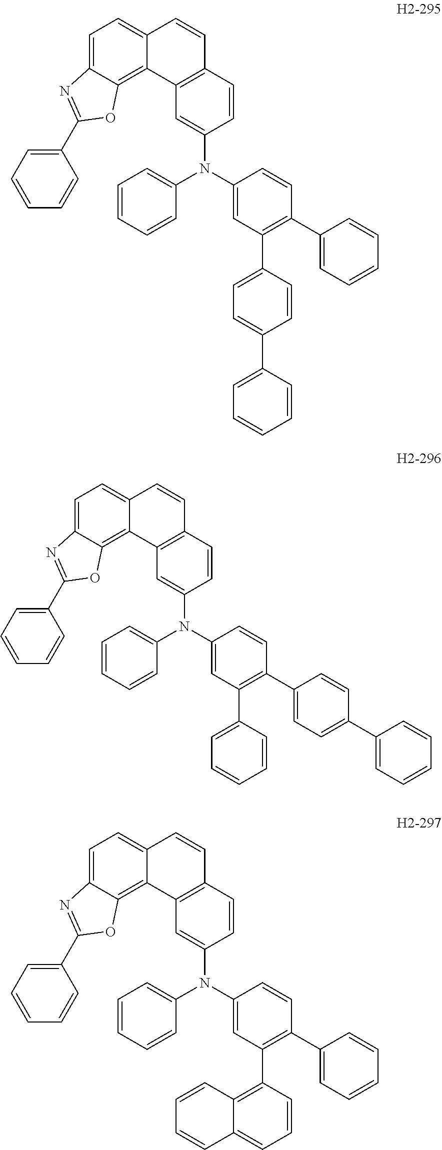 [SMILES: c1ccc(-c2ccc(-c3cc(N(c4ccccc4)c4ccc5ccc6ccc7nc(-c8ccccc8)oc7c6c5c4)ccc3-c3ccccc3)cc2)cc1 ; c1ccc(-c2ccc(-c3ccc(N(c4ccccc4)c4ccc5ccc6ccc7nc(-c8ccccc8)oc7c6c5c4)cc3-c3ccccc3)cc2)cc1 ; c1ccc(-c2nc3ccc4ccc5ccc(N(c6ccccc6)c6ccc(-c7ccccc7)c(-c7cccc8ccccc78)c6)cc5c4c3o2)cc1]

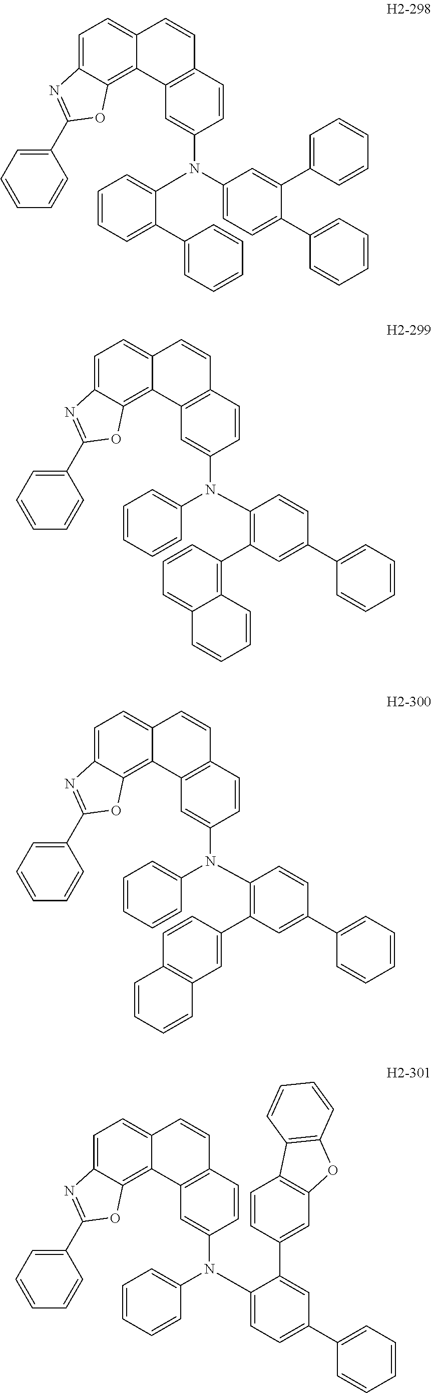 [SMILES: c1ccc(-c2nc3ccc4ccc5ccc(N(c6ccc(-c7ccccc7)c(-c7ccccc7)c6)c6ccccc6-c6ccccc6)cc5c4c3o2)cc1 ; c1ccc(-c2ccc(N(c3ccccc3)c3ccc4ccc5ccc6nc(-c7ccccc7)oc6c5c4c3)c(-c3cccc4ccccc34)c2)cc1 ; c1ccc(-c2ccc(N(c3ccccc3)c3ccc4ccc5ccc6nc(-c7ccccc7)oc6c5c4c3)c(-c3ccc4ccccc4c3)c2)cc1 ; c1ccc(-c2ccc(N(c3ccccc3)c3ccc4ccc5ccc6nc(-c7ccccc7)oc6c5c4c3)c(-c3ccc4c(c3)oc3ccccc34)c2)cc1]

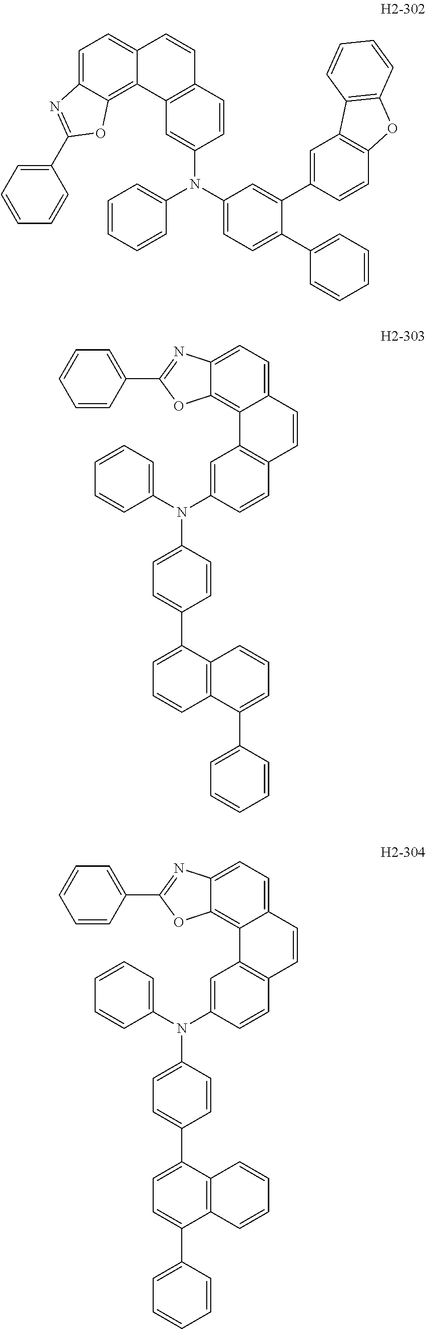 [SMILES: c1ccc(-c2nc3ccc4ccc5ccc(N(c6ccccc6)c6ccc(-c7ccccc7)c(-c7ccc8oc9ccccc9c8c7)c6)cc5c4c3o2)cc1 ; c1ccc(-c2nc3ccc4ccc5ccc(N(c6ccccc6)c6ccc(-c7cccc8c(-c9ccccc9)cccc78)cc6)cc5c4c3o2)cc1 ; c1ccc(-c2nc3ccc4ccc5ccc(N(c6ccccc6)c6ccc(-c7ccc(-c8ccccc8)c8ccccc78)cc6)cc5c4c3o2)cc1]

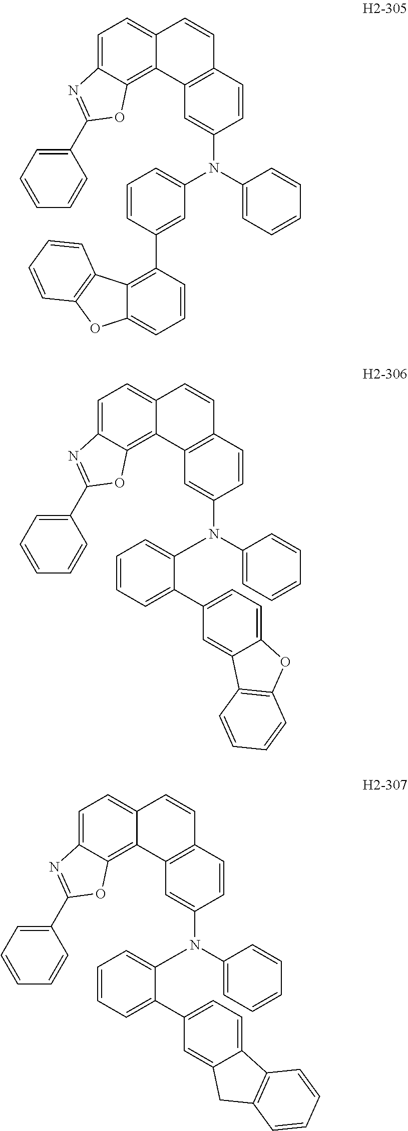 [SMILES: c1ccc(-c2nc3ccc4ccc5ccc(N(c6ccccc6)c6cccc(-c7cccc8oc9ccccc9c78)c6)cc5c4c3o2)cc1 ; c1ccc(-c2nc3ccc4ccc5ccc(N(c6ccccc6)c6ccccc6-c6ccc7oc8ccccc8c7c6)cc5c4c3o2)cc1 ; c1ccc(-c2nc3ccc4ccc5ccc(N(c6ccccc6)c6ccccc6-c6ccc7c(c6)Cc6ccccc6-7)cc5c4c3o2)cc1]

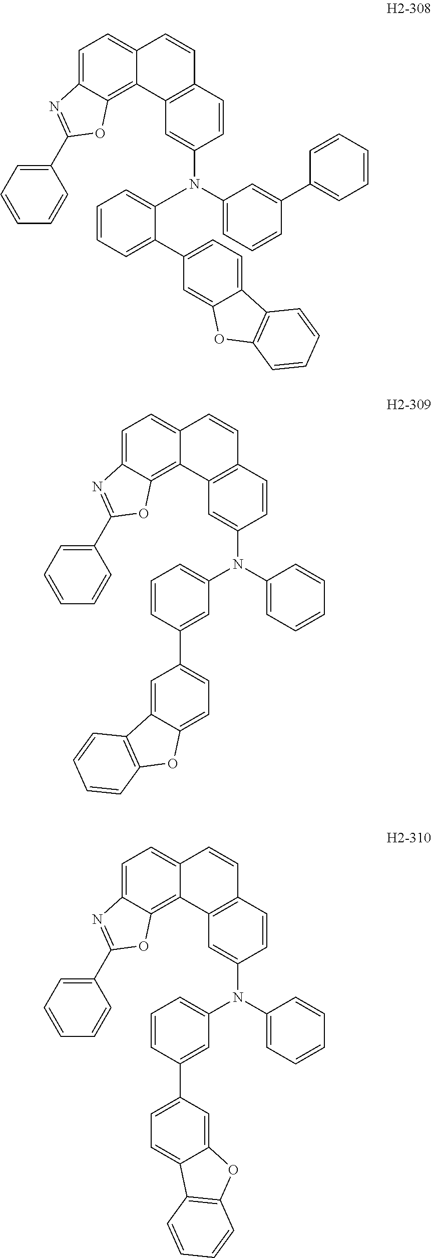 [SMILES: c1ccc(-c2cccc(N(c3ccc4ccc5ccc6nc(-c7ccccc7)oc6c5c4c3)c3ccccc3-c3ccc4c(c3)oc3ccccc34)c2)cc1 ; c1ccc(-c2nc3ccc4ccc5ccc(N(c6ccccc6)c6cccc(-c7ccc8oc9ccccc9c8c7)c6)cc5c4c3o2)cc1 ; c1ccc(-c2nc3ccc4ccc5ccc(N(c6ccccc6)c6cccc(-c7ccc8c(c7)oc7ccccc78)c6)cc5c4c3o2)cc1]

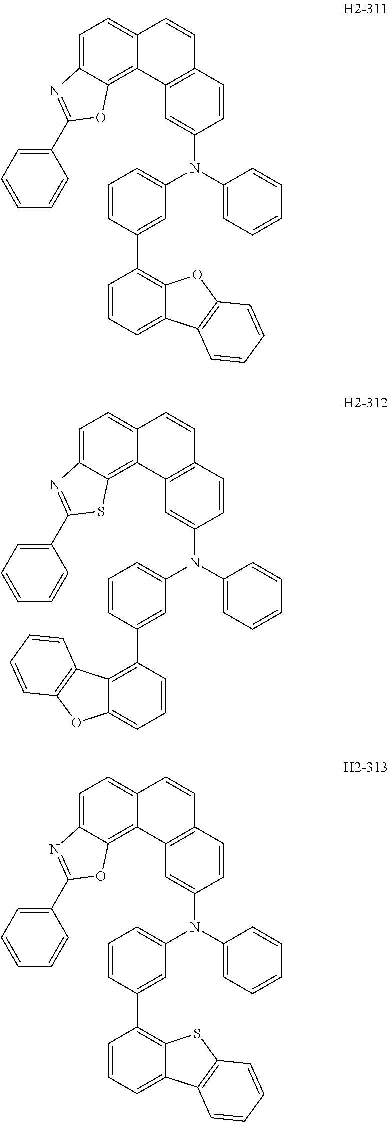 [SMILES: c1ccc(-c2nc3ccc4ccc5ccc(N(c6ccccc6)c6cccc(-c7cccc8c7oc7ccccc78)c6)cc5c4c3o2)cc1 ; c1ccc(-c2nc3ccc4ccc5ccc(N(c6ccccc6)c6cccc(-c7cccc8oc9ccccc9c78)c6)cc5c4c3s2)cc1 ; c1ccc(-c2nc3ccc4ccc5ccc(N(c6ccccc6)c6cccc(-c7cccc8c7sc7ccccc78)c6)cc5c4c3o2)cc1]

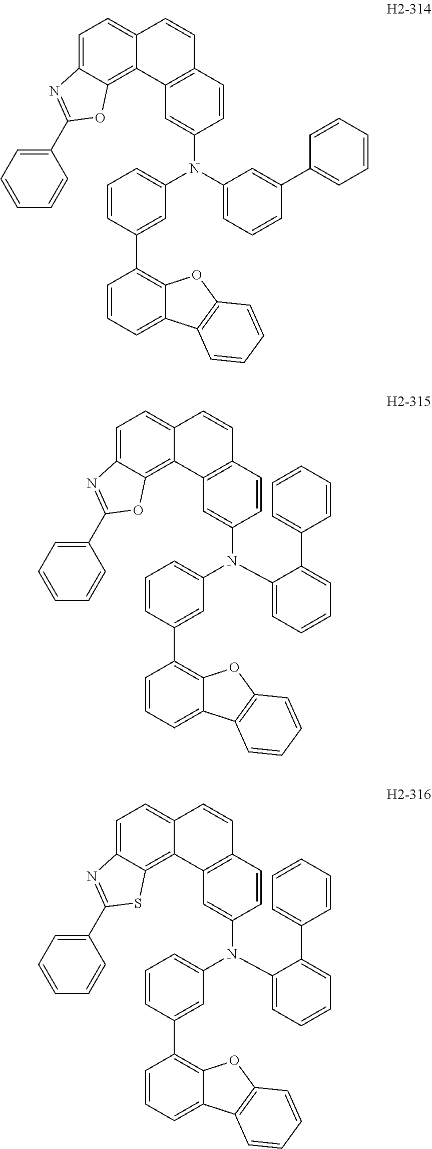 [SMILES: c1ccc(-c2cccc(N(c3cccc(-c4cccc5c4oc4ccccc45)c3)c3ccc4ccc5ccc6nc(-c7ccccc7)oc6c5c4c3)c2)cc1 ; c1ccc(-c2nc3ccc4ccc5ccc(N(c6cccc(-c7cccc8c7oc7ccccc78)c6)c6ccccc6-c6ccccc6)cc5c4c3o2)cc1 ; c1ccc(-c2nc3ccc4ccc5ccc(N(c6cccc(-c7cccc8c7oc7ccccc78)c6)c6ccccc6-c6ccccc6)cc5c4c3s2)cc1]

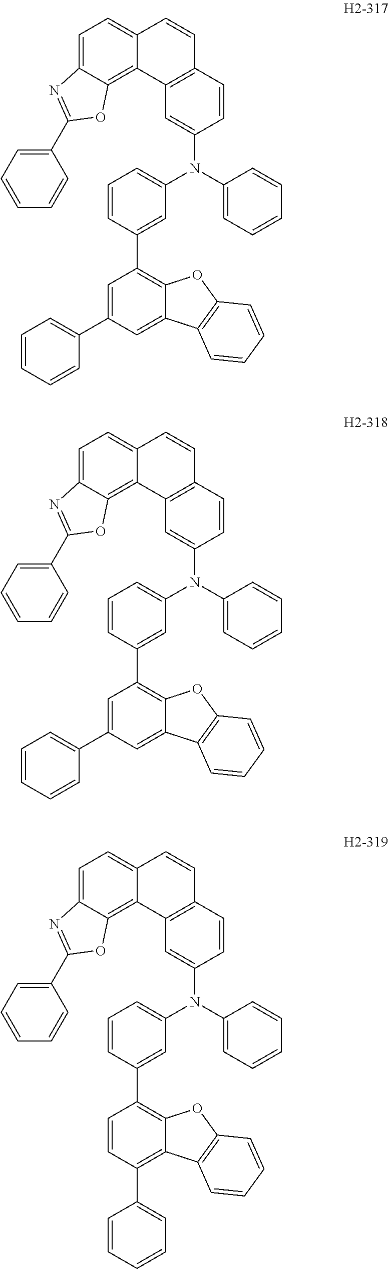 [SMILES: c1ccc(-c2cc(-c3cccc(N(c4ccccc4)c4ccc5ccc6ccc7nc(-c8ccccc8)oc7c6c5c4)c3)c3oc4ccccc4c3c2)cc1 ; c1ccc(-c2cc(-c3cccc(N(c4ccccc4)c4ccc5ccc6ccc7nc(-c8ccccc8)oc7c6c5c4)c3)c3oc4ccccc4c3c2)cc1 ; c1ccc(-c2nc3ccc4ccc5ccc(N(c6ccccc6)c6cccc(-c7ccc(-c8ccccc8)c8c7oc7ccccc78)c6)cc5c4c3o2)cc1]

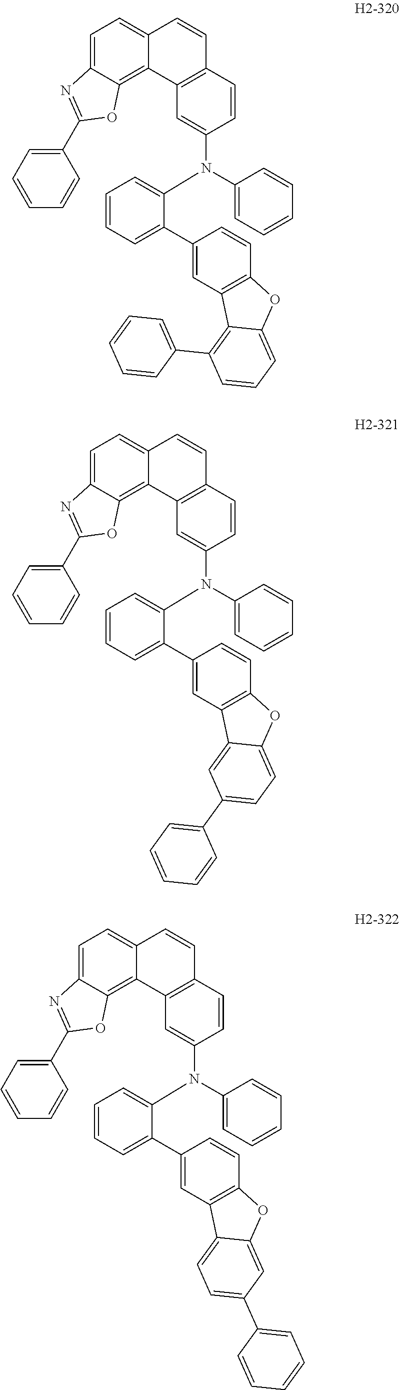 [SMILES: c1ccc(-c2nc3ccc4ccc5ccc(N(c6ccccc6)c6ccccc6-c6ccc7oc8cccc(-c9ccccc9)c8c7c6)cc5c4c3o2)cc1 ; c1ccc(-c2ccc3oc4ccc(-c5ccccc5N(c5ccccc5)c5ccc6ccc7ccc8nc(-c9ccccc9)oc8c7c6c5)cc4c3c2)cc1 ; c1ccc(-c2ccc3c(c2)oc2ccc(-c4ccccc4N(c4ccccc4)c4ccc5ccc6ccc7nc(-c8ccccc8)oc7c6c5c4)cc23)cc1]

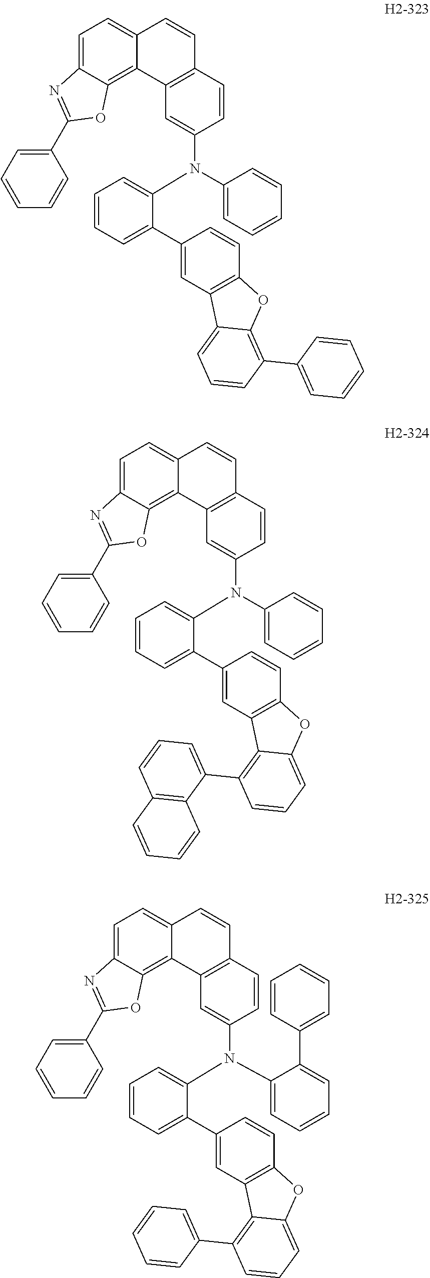 [SMILES: c1ccc(-c2nc3ccc4ccc5ccc(N(c6ccccc6)c6ccccc6-c6ccc7oc8c(-c9ccccc9)cccc8c7c6)cc5c4c3o2)cc1 ; c1ccc(-c2nc3ccc4ccc5ccc(N(c6ccccc6)c6ccccc6-c6ccc7oc8cccc(-c9cccc%10ccccc9%10)c8c7c6)cc5c4c3o2)cc1 ; c1ccc(-c2nc3ccc4ccc5ccc(N(c6ccccc6-c6ccccc6)c6ccccc6-c6ccc7oc8cccc(-c9ccccc9)c8c7c6)cc5c4c3o2)cc1]

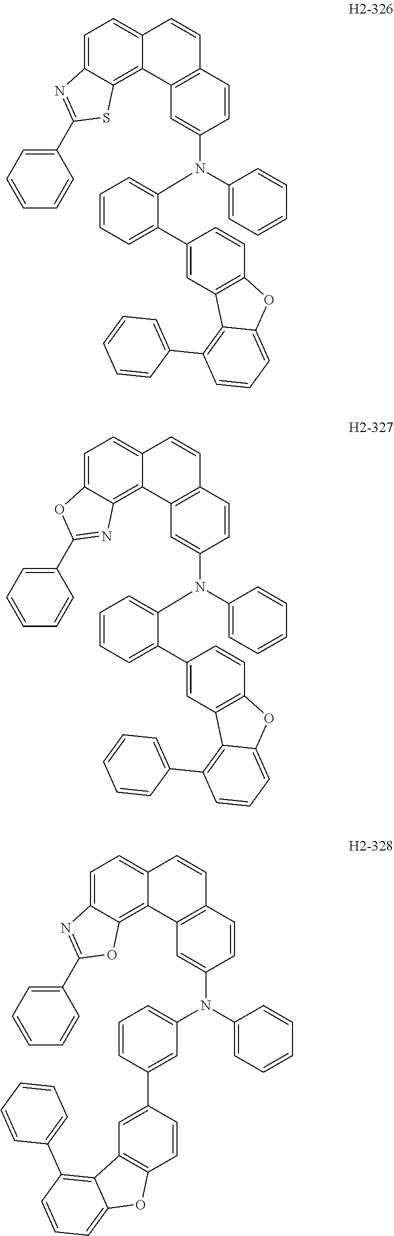 [SMILES: c1ccc(-c2nc3ccc4ccc5ccc(N(c6ccccc6)c6ccccc6-c6ccc7oc8cccc(-c9ccccc9)c8c7c6)cc5c4c3s2)cc1 ; c1ccc(-c2nc3c(ccc4ccc5ccc(N(c6ccccc6)c6ccccc6-c6ccc7oc8cccc(-c9ccccc9)c8c7c6)cc5c43)o2)cc1 ; c1ccc(-c2nc3ccc4ccc5ccc(N(c6ccccc6)c6cccc(-c7ccc8oc9cccc(-c%10ccccc%10)c9c8c7)c6)cc5c4c3o2)cc1]

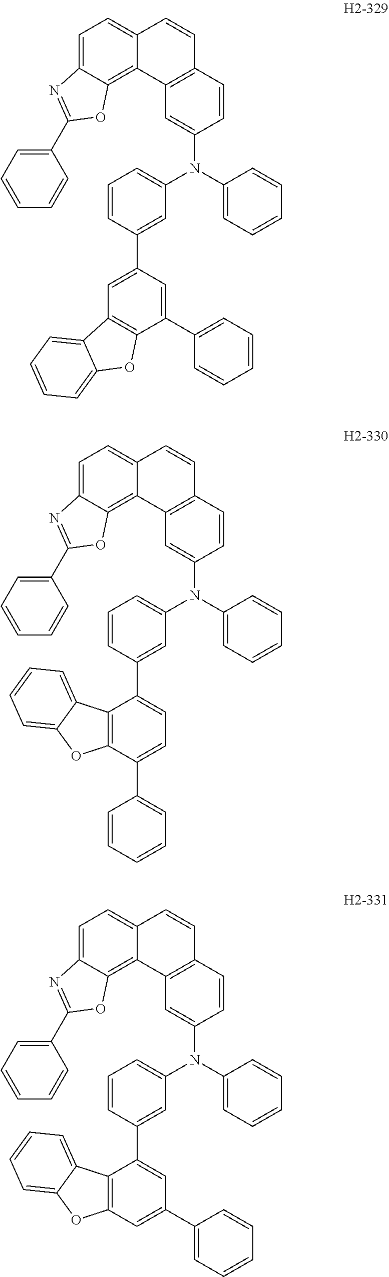 [SMILES: c1ccc(-c2nc3ccc4ccc5ccc(N(c6ccccc6)c6cccc(-c7cc(-c8ccccc8)c8oc9ccccc9c8c7)c6)cc5c4c3o2)cc1 ; c1ccc(-c2nc3ccc4ccc5ccc(N(c6ccccc6)c6cccc(-c7ccc(-c8ccccc8)c8oc9ccccc9c78)c6)cc5c4c3o2)cc1 ; c1ccc(-c2cc(-c3cccc(N(c4ccccc4)c4ccc5ccc6ccc7nc(-c8ccccc8)oc7c6c5c4)c3)c3c(c2)oc2ccccc23)cc1]

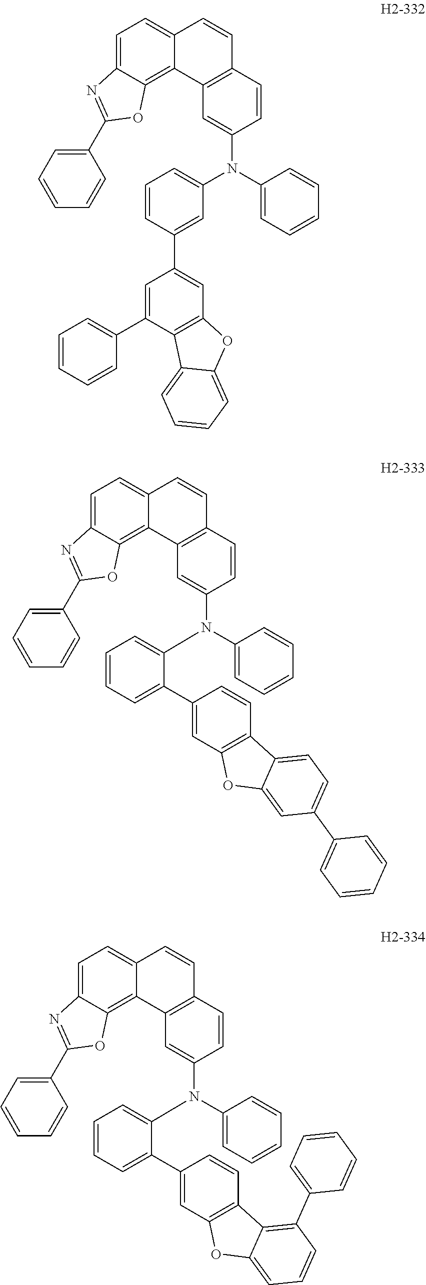 [SMILES: c1ccc(-c2nc3ccc4ccc5ccc(N(c6ccccc6)c6cccc(-c7cc(-c8ccccc8)c8c(c7)oc7ccccc78)c6)cc5c4c3o2)cc1 ; c1ccc(-c2ccc3c(c2)oc2cc(-c4ccccc4N(c4ccccc4)c4ccc5ccc6ccc7nc(-c8ccccc8)oc7c6c5c4)ccc23)cc1 ; c1ccc(-c2nc3ccc4ccc5ccc(N(c6ccccc6)c6ccccc6-c6ccc7c(c6)oc6cccc(-c8ccccc8)c67)cc5c4c3o2)cc1]

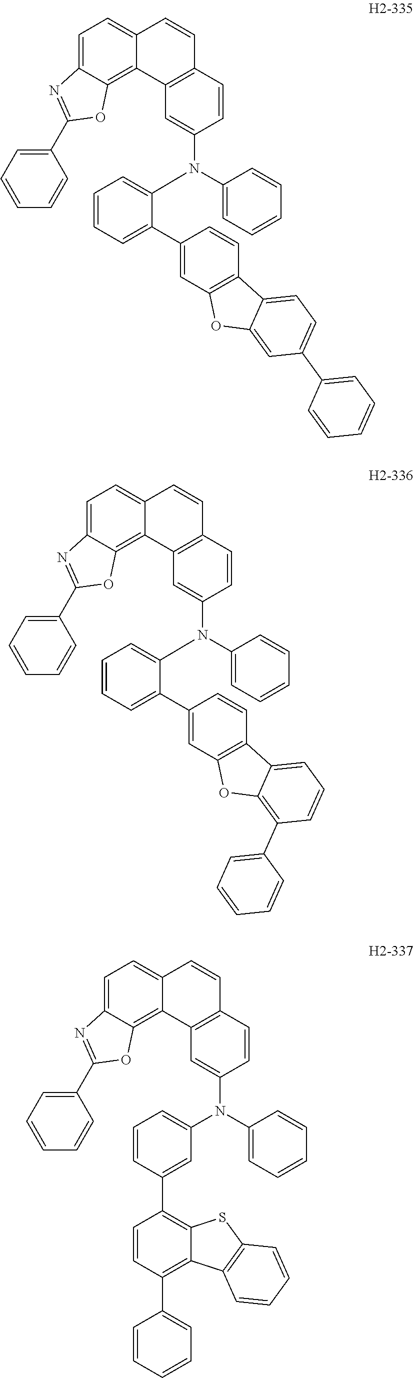 [SMILES: c1ccc(-c2ccc3c(c2)oc2cc(-c4ccccc4N(c4ccccc4)c4ccc5ccc6ccc7nc(-c8ccccc8)oc7c6c5c4)ccc23)cc1 ; c1ccc(-c2nc3ccc4ccc5ccc(N(c6ccccc6)c6ccccc6-c6ccc7c(c6)oc6c(-c8ccccc8)cccc67)cc5c4c3o2)cc1 ; c1ccc(-c2nc3ccc4ccc5ccc(N(c6ccccc6)c6cccc(-c7ccc(-c8ccccc8)c8c7sc7ccccc78)c6)cc5c4c3o2)cc1]

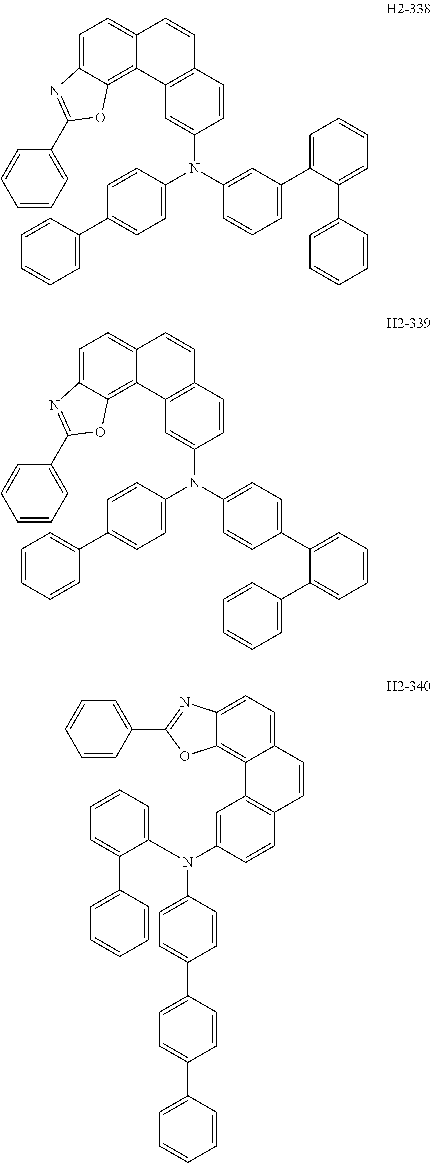 [SMILES: c1ccc(-c2ccc(N(c3cccc(-c4ccccc4-c4ccccc4)c3)c3ccc4ccc5ccc6nc(-c7ccccc7)oc6c5c4c3)cc2)cc1 ; c1ccc(-c2ccc(N(c3ccc(-c4ccccc4-c4ccccc4)cc3)c3ccc4ccc5ccc6nc(-c7ccccc7)oc6c5c4c3)cc2)cc1 ; c1ccc(-c2ccc(-c3ccc(N(c4ccc5ccc6ccc7nc(-c8ccccc8)oc7c6c5c4)c4ccccc4-c4ccccc4)cc3)cc2)cc1]

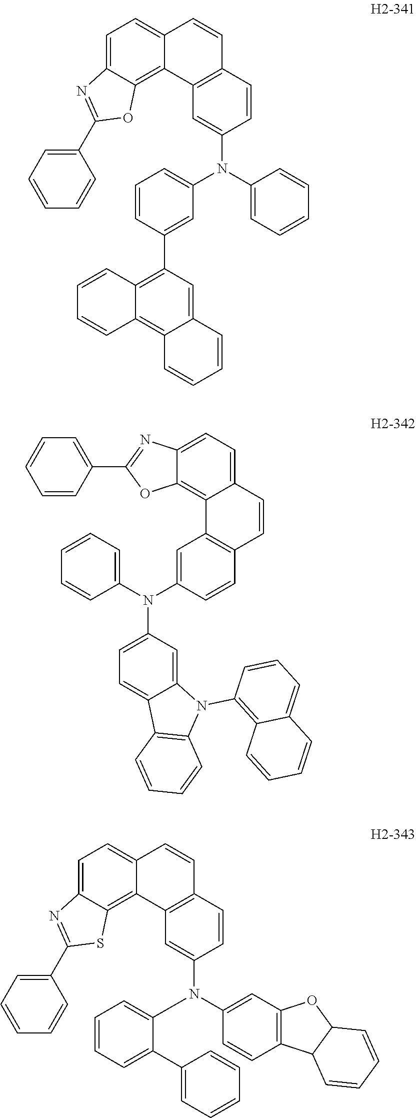 [SMILES: c1ccc(-c2nc3ccc4ccc5ccc(N(c6ccccc6)c6cccc(-c7cc8ccccc8c8ccccc78)c6)cc5c4c3o2)cc1 ; c1ccc(-c2nc3ccc4ccc5ccc(N(c6ccccc6)c6ccc7c8ccccc8n(-c8cccc9ccccc89)c7c6)cc5c4c3o2)cc1 ; C1=CC2Oc3cc(N(c4ccc5ccc6ccc7nc(-c8ccccc8)sc7c6c5c4)c4ccccc4-c4ccccc4)ccc3C2C=C1]

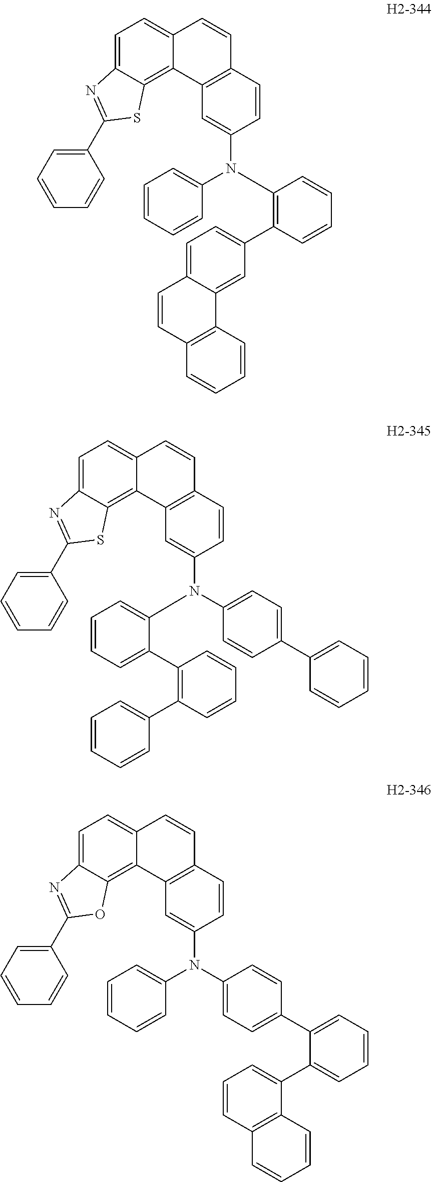 [SMILES: c1ccc(-c2nc3ccc4ccc5ccc(N(c6ccccc6)c6ccccc6-c6ccc7ccc8ccccc8c7c6)cc5c4c3s2)cc1 ; c1ccc(-c2ccc(N(c3ccc4ccc5ccc6nc(-c7ccccc7)sc6c5c4c3)c3ccccc3-c3ccccc3-c3ccccc3)cc2)cc1 ; c1ccc(-c2nc3ccc4ccc5ccc(N(c6ccccc6)c6ccc(-c7ccccc7-c7cccc8ccccc78)cc6)cc5c4c3o2)cc1]

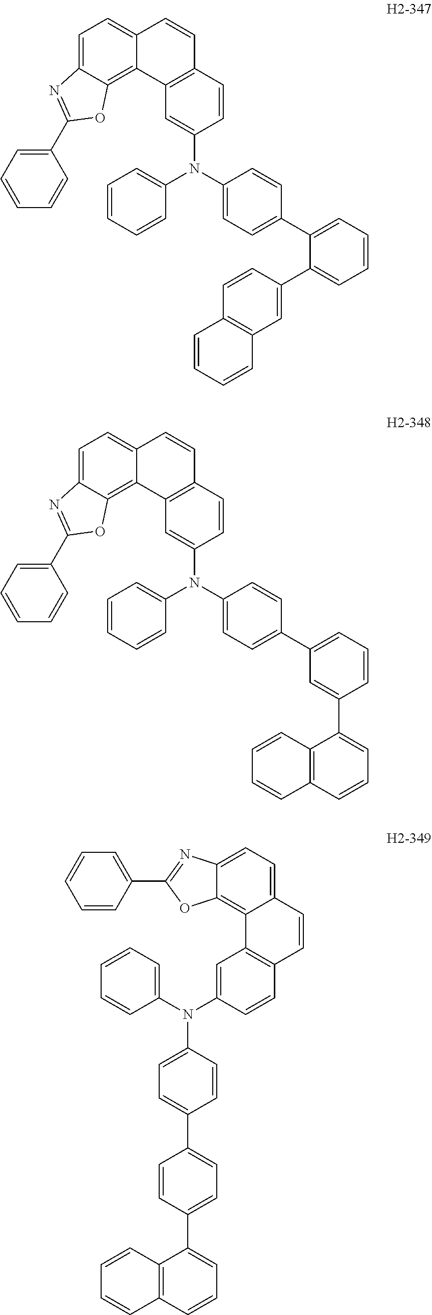 [SMILES: c1ccc(-c2nc3ccc4ccc5ccc(N(c6ccccc6)c6ccc(-c7ccccc7-c7ccc8ccccc8c7)cc6)cc5c4c3o2)cc1 ; c1ccc(-c2nc3ccc4ccc5ccc(N(c6ccccc6)c6ccc(-c7cccc(-c8cccc9ccccc89)c7)cc6)cc5c4c3o2)cc1 ; c1ccc(-c2nc3ccc4ccc5ccc(N(c6ccccc6)c6ccc(-c7ccc(-c8cccc9ccccc89)cc7)cc6)cc5c4c3o2)cc1]

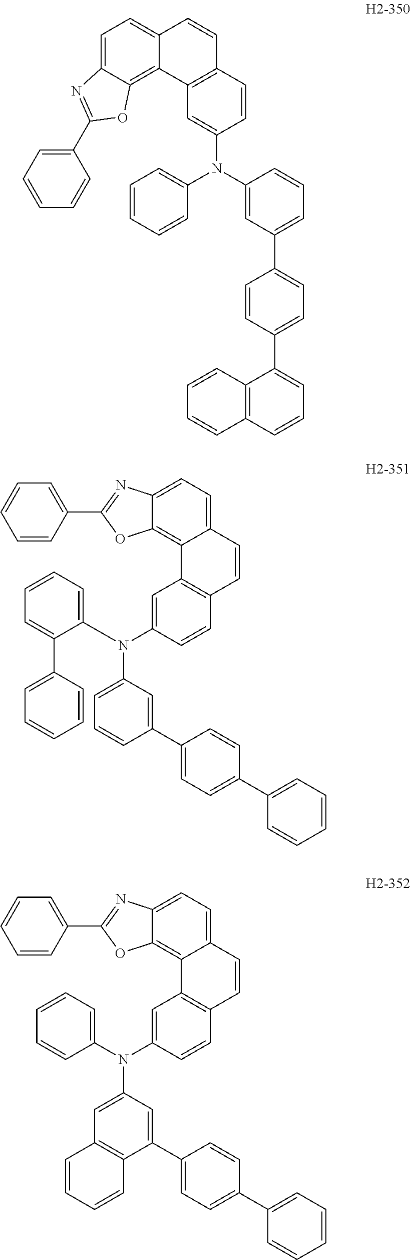 [SMILES: c1ccc(-c2nc3ccc4ccc5ccc(N(c6ccccc6)c6cccc(-c7ccc(-c8cccc9ccccc89)cc7)c6)cc5c4c3o2)cc1 ; c1ccc(-c2ccc(-c3cccc(N(c4ccc5ccc6ccc7nc(-c8ccccc8)oc7c6c5c4)c4ccccc4-c4ccccc4)c3)cc2)cc1 ; c1ccc(-c2ccc(-c3cc(N(c4ccccc4)c4ccc5ccc6ccc7nc(-c8ccccc8)oc7c6c5c4)cc4ccccc34)cc2)cc1]

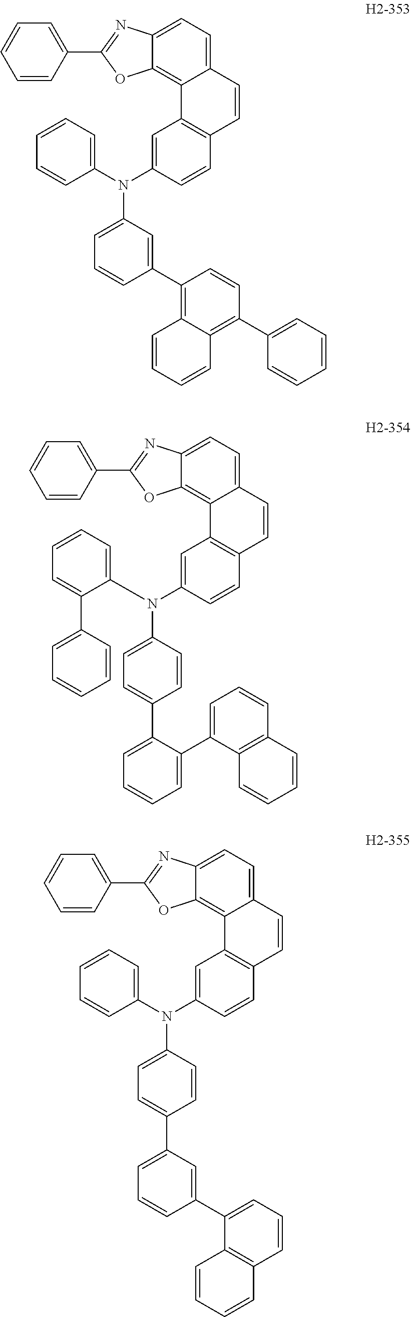 [SMILES: c1ccc(-c2nc3ccc4ccc5ccc(N(c6ccccc6)c6cccc(-c7ccc(-c8ccccc8)c8ccccc78)c6)cc5c4c3o2)cc1 ; c1ccc(-c2nc3ccc4ccc5ccc(N(c6ccc(-c7ccccc7-c7cccc8ccccc78)cc6)c6ccccc6-c6ccccc6)cc5c4c3o2)cc1 ; c1ccc(-c2nc3ccc4ccc5ccc(N(c6ccccc6)c6ccc(-c7cccc(-c8cccc9ccccc89)c7)cc6)cc5c4c3o2)cc1]

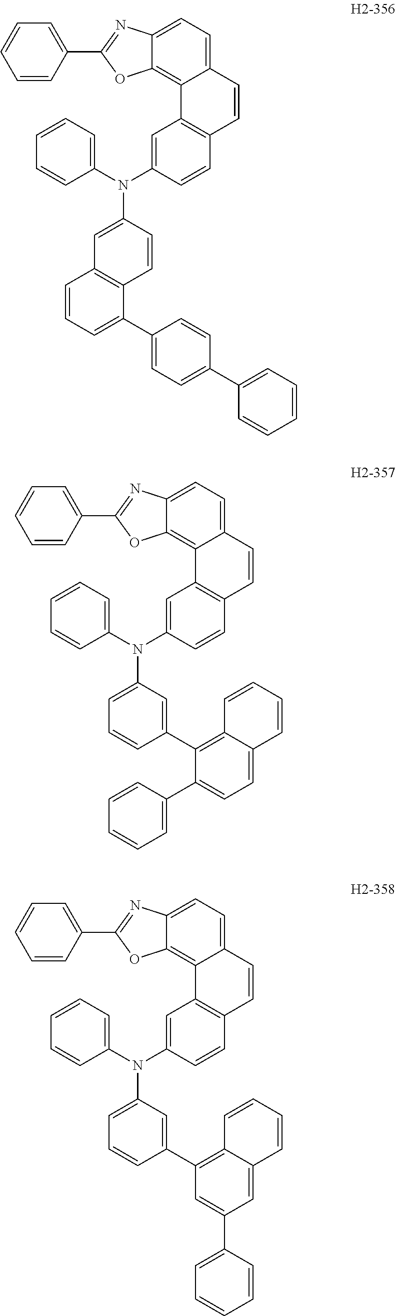 [SMILES: c1ccc(-c2ccc(-c3cccc4cc(N(c5ccccc5)c5ccc6ccc7ccc8nc(-c9ccccc9)oc8c7c6c5)ccc34)cc2)cc1 ; c1ccc(-c2nc3ccc4ccc5ccc(N(c6ccccc6)c6cccc(-c7c(-c8ccccc8)ccc8ccccc78)c6)cc5c4c3o2)cc1 ; c1ccc(-c2cc(-c3cccc(N(c4ccccc4)c4ccc5ccc6ccc7nc(-c8ccccc8)oc7c6c5c4)c3)c3ccccc3c2)cc1]

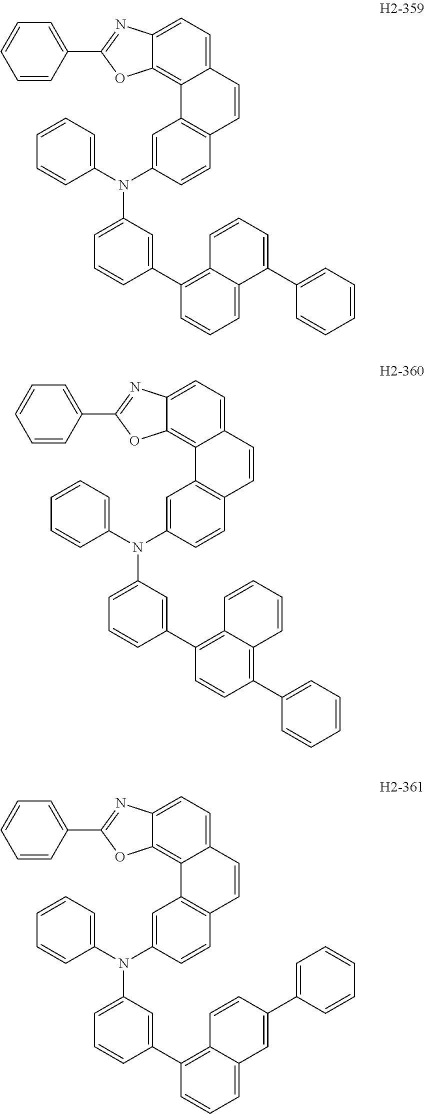 [SMILES: c1ccc(-c2nc3ccc4ccc5ccc(N(c6ccccc6)c6cccc(-c7cccc8c(-c9ccccc9)cccc78)c6)cc5c4c3o2)cc1 ; c1ccc(-c2nc3ccc4ccc5ccc(N(c6ccccc6)c6cccc(-c7ccc(-c8ccccc8)c8ccccc78)c6)cc5c4c3o2)cc1 ; c1ccc(-c2ccc3c(-c4cccc(N(c5ccccc5)c5ccc6ccc7ccc8nc(-c9ccccc9)oc8c7c6c5)c4)cccc3c2)cc1]

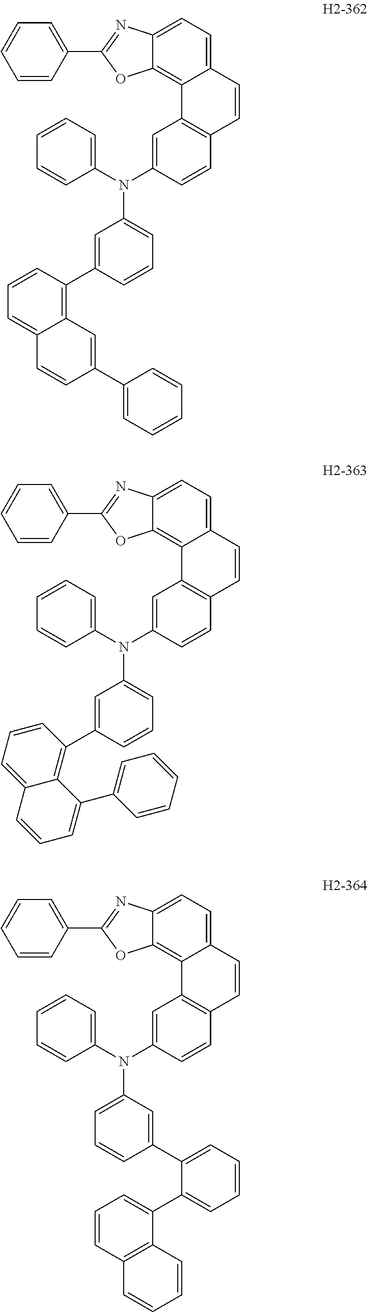 [SMILES: c1ccc(-c2ccc3cccc(-c4cccc(N(c5ccccc5)c5ccc6ccc7ccc8nc(-c9ccccc9)oc8c7c6c5)c4)c3c2)cc1 ; c1ccc(-c2nc3ccc4ccc5ccc(N(c6ccccc6)c6cccc(-c7cccc8cccc(-c9ccccc9)c78)c6)cc5c4c3o2)cc1 ; c1ccc(-c2nc3ccc4ccc5ccc(N(c6ccccc6)c6cccc(-c7ccccc7-c7cccc8ccccc78)c6)cc5c4c3o2)cc1]

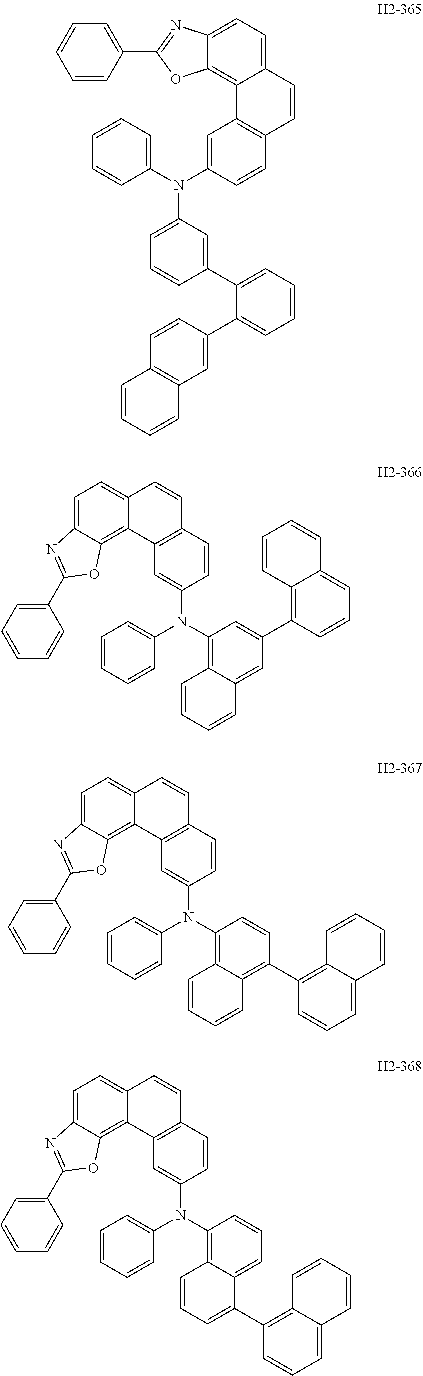 [SMILES: c1ccc(-c2nc3ccc4ccc5ccc(N(c6ccccc6)c6cccc(-c7ccccc7-c7ccc8ccccc8c7)c6)cc5c4c3o2)cc1 ; c1ccc(-c2nc3ccc4ccc5ccc(N(c6ccccc6)c6cc(-c7cccc8ccccc78)cc7ccccc67)cc5c4c3o2)cc1 ; c1ccc(-c2nc3ccc4ccc5ccc(N(c6ccccc6)c6ccc(-c7cccc8ccccc78)c7ccccc67)cc5c4c3o2)cc1 ; c1ccc(-c2nc3ccc4ccc5ccc(N(c6ccccc6)c6cccc7c(-c8cccc9ccccc89)cccc67)cc5c4c3o2)cc1]

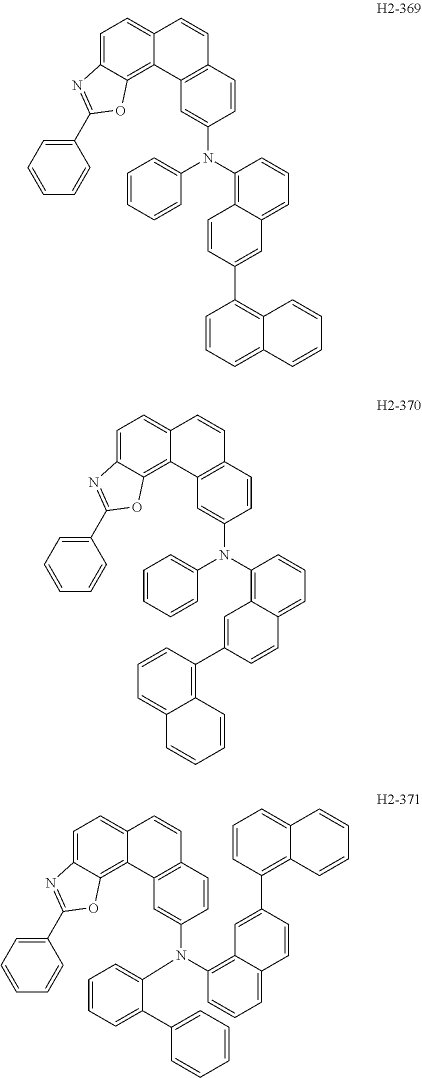 [SMILES: c1ccc(-c2nc3ccc4ccc5ccc(N(c6ccccc6)c6cccc7cc(-c8cccc9ccccc89)ccc67)cc5c4c3o2)cc1 ; c1ccc(-c2nc3ccc4ccc5ccc(N(c6ccccc6)c6cccc7ccc(-c8cccc9ccccc89)cc67)cc5c4c3o2)cc1 ; c1ccc(-c2nc3ccc4ccc5ccc(N(c6ccccc6-c6ccccc6)c6cccc7ccc(-c8cccc9ccccc89)cc67)cc5c4c3o2)cc1]

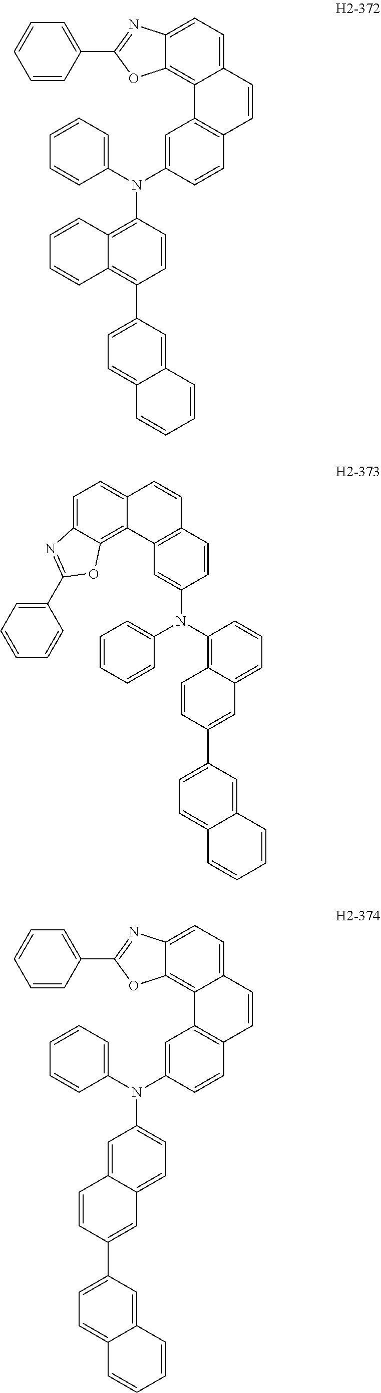 [SMILES: c1ccc(-c2nc3ccc4ccc5ccc(N(c6ccccc6)c6ccc(-c7ccc8ccccc8c7)c7ccccc67)cc5c4c3o2)cc1 ; c1ccc(-c2nc3ccc4ccc5ccc(N(c6ccccc6)c6cccc7cc(-c8ccc9ccccc9c8)ccc67)cc5c4c3o2)cc1 ; c1ccc(-c2nc3ccc4ccc5ccc(N(c6ccccc6)c6ccc7cc(-c8ccc9ccccc9c8)ccc7c6)cc5c4c3o2)cc1]

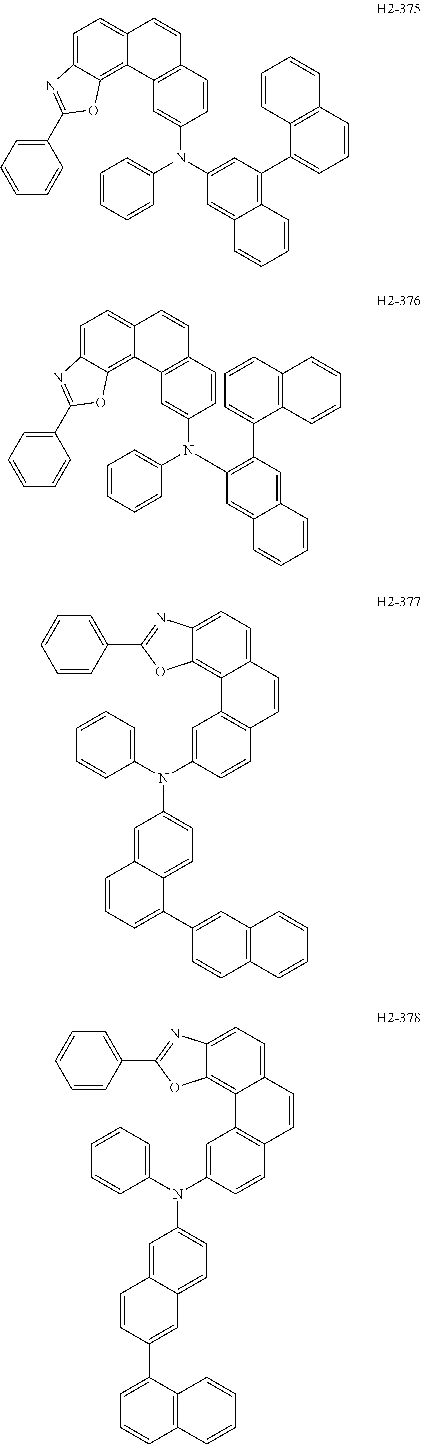 [SMILES: c1ccc(-c2nc3ccc4ccc5ccc(N(c6ccccc6)c6cc(-c7cccc8ccccc78)c7ccccc7c6)cc5c4c3o2)cc1 ; c1ccc(-c2nc3ccc4ccc5ccc(N(c6ccccc6)c6cc7ccccc7cc6-c6cccc7ccccc67)cc5c4c3o2)cc1 ; c1ccc(-c2nc3ccc4ccc5ccc(N(c6ccccc6)c6ccc7c(-c8ccc9ccccc9c8)cccc7c6)cc5c4c3o2)cc1 ; c1ccc(-c2nc3ccc4ccc5ccc(N(c6ccccc6)c6ccc7cc(-c8cccc9ccccc89)ccc7c6)cc5c4c3o2)cc1]

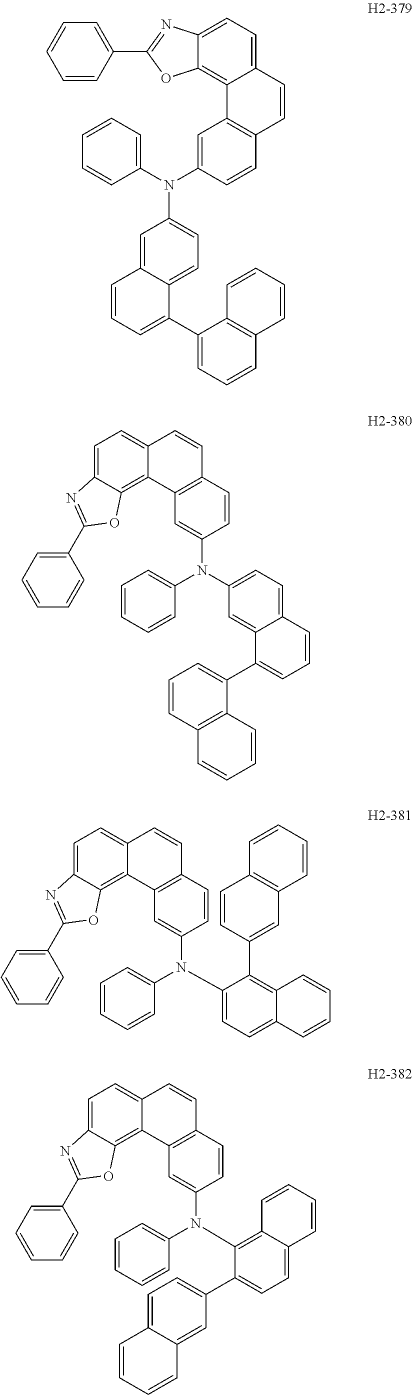 [SMILES: c1ccc(-c2nc3ccc4ccc5ccc(N(c6ccccc6)c6ccc7c(-c8cccc9ccccc89)cccc7c6)cc5c4c3o2)cc1 ; c1ccc(-c2nc3ccc4ccc5ccc(N(c6ccccc6)c6ccc7cccc(-c8cccc9ccccc89)c7c6)cc5c4c3o2)cc1 ; c1ccc(-c2nc3ccc4ccc5ccc(N(c6ccccc6)c6ccc7ccccc7c6-c6ccc7ccccc7c6)cc5c4c3o2)cc1 ; c1ccc(-c2nc3ccc4ccc5ccc(N(c6ccccc6)c6c(-c7ccc8ccccc8c7)ccc7ccccc67)cc5c4c3o2)cc1]

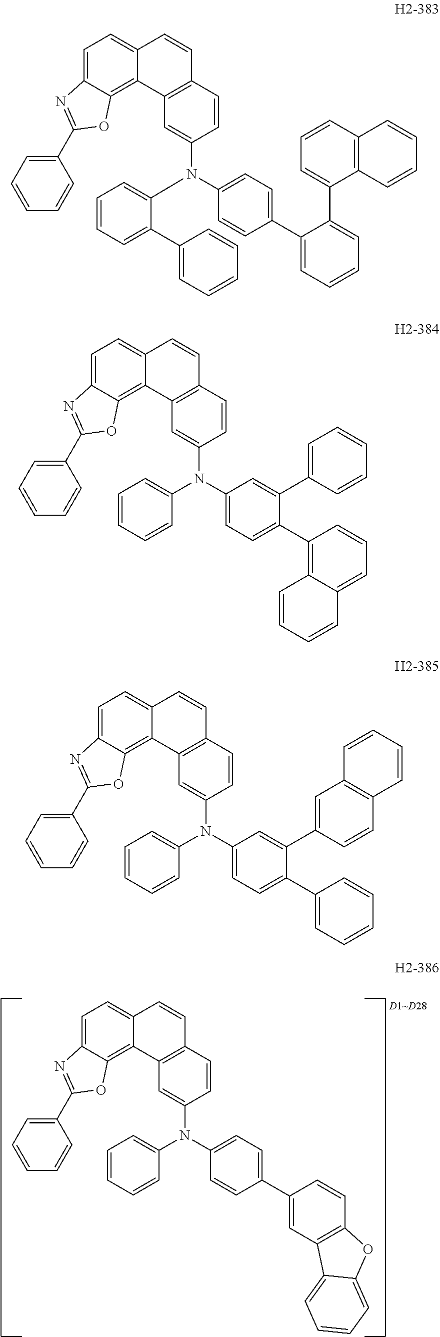

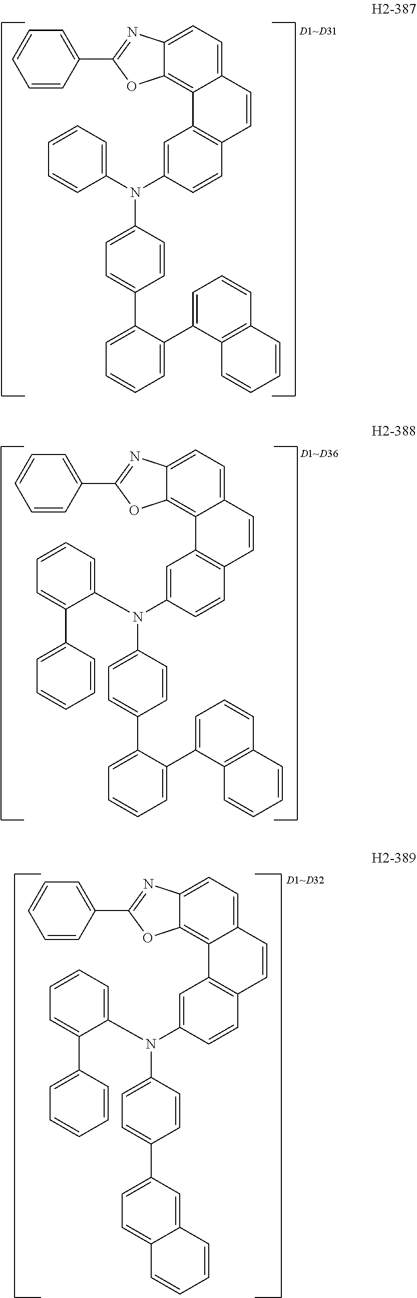

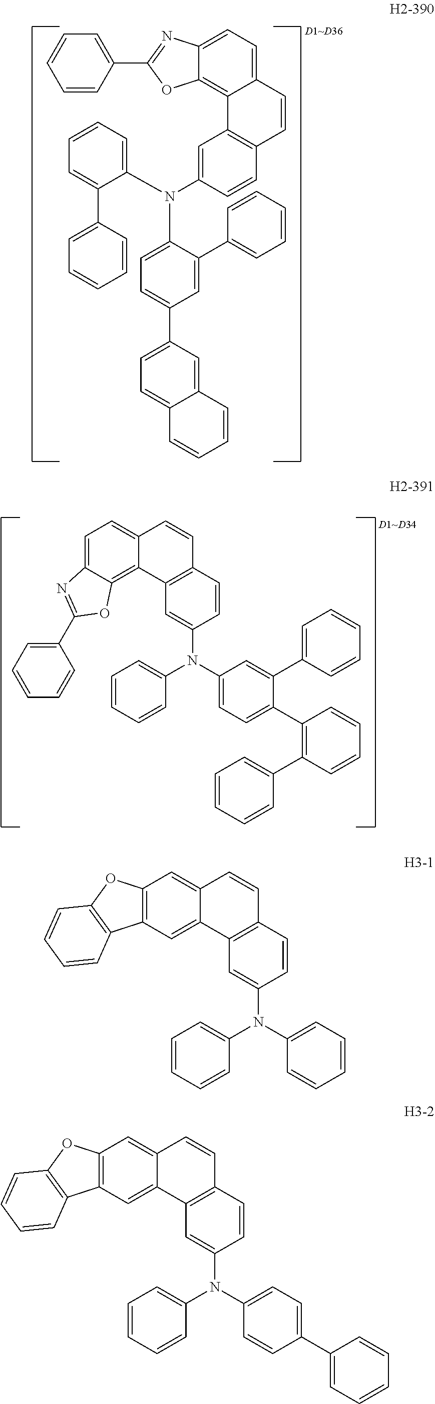

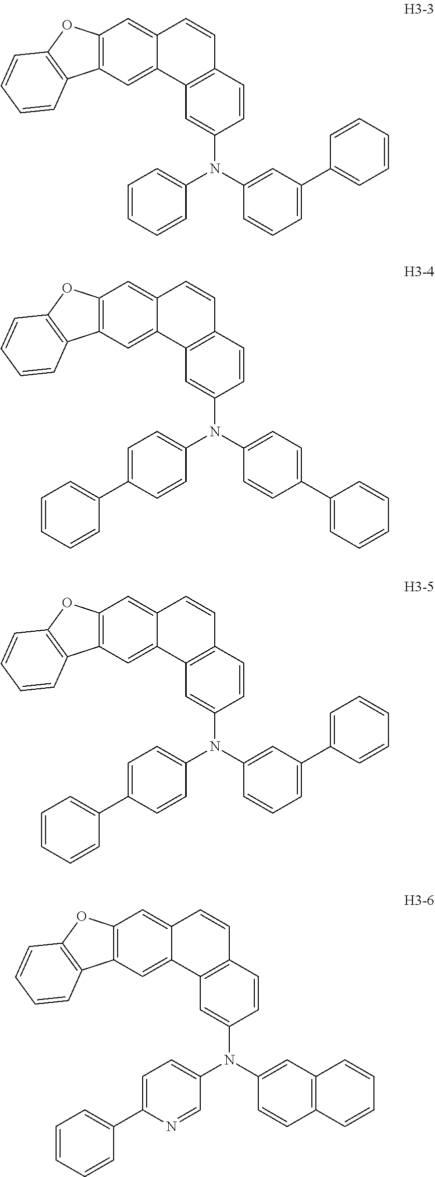 [SMILES: c1ccc(-c2cccc(N(c3ccccc3)c3ccc4ccc5cc6oc7ccccc7c6cc5c4c3)c2)cc1 ; c1ccc(-c2ccc(N(c3ccc(-c4ccccc4)cc3)c3ccc4ccc5cc6oc7ccccc7c6cc5c4c3)cc2)cc1 ; c1ccc(-c2ccc(N(c3cccc(-c4ccccc4)c3)c3ccc4ccc5cc6oc7ccccc7c6cc5c4c3)cc2)cc1 ; c1ccc(-c2ccc(N(c3ccc4ccccc4c3)c3ccc4ccc5cc6oc7ccccc7c6cc5c4c3)cn2)cc1]

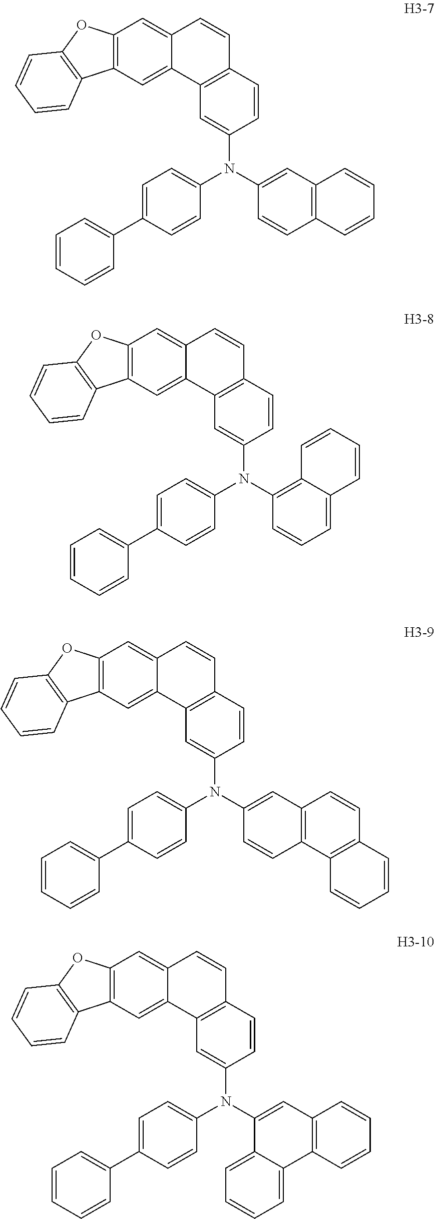 [SMILES: c1ccc(-c2ccc(N(c3ccc4ccccc4c3)c3ccc4ccc5cc6oc7ccccc7c6cc5c4c3)cc2)cc1 ; c1ccc(-c2ccc(N(c3ccc4ccc5cc6oc7ccccc7c6cc5c4c3)c3cccc4ccccc34)cc2)cc1 ; c1ccc(-c2ccc(N(c3ccc4c(ccc5ccccc54)c3)c3ccc4ccc5cc6oc7ccccc7c6cc5c4c3)cc2)cc1 ; c1ccc(-c2ccc(N(c3ccc4ccc5cc6oc7ccccc7c6cc5c4c3)c3cc4ccccc4c4ccccc34)cc2)cc1]

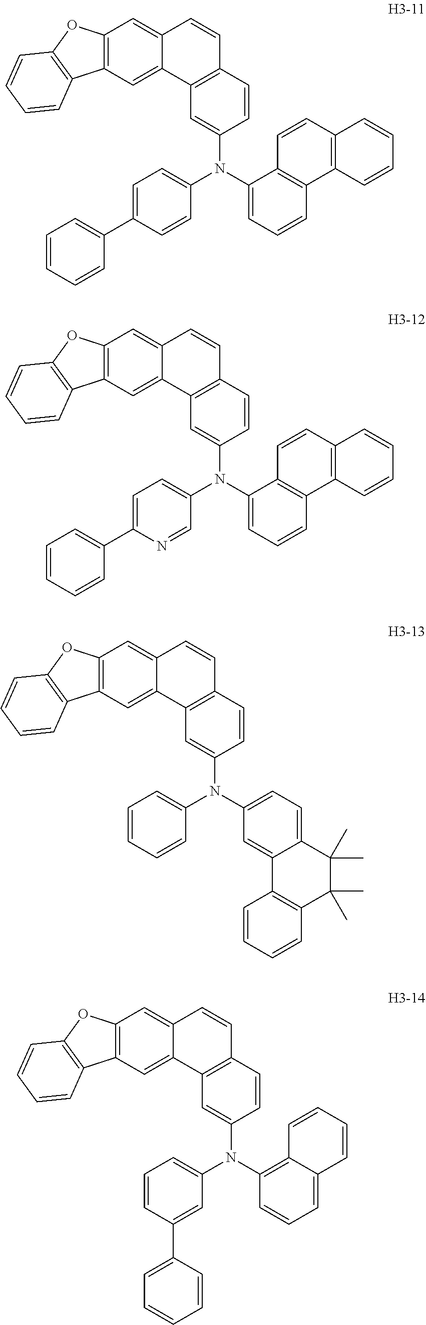 [SMILES: c1ccc(-c2ccc(N(c3ccc4ccc5cc6oc7ccccc7c6cc5c4c3)c3cccc4c3ccc3ccccc34)cc2)cc1 ; c1ccc(-c2ccc(N(c3ccc4ccc5cc6oc7ccccc7c6cc5c4c3)c3cccc4c3ccc3ccccc34)cn2)cc1 ; CC1(C)c2ccccc2-c2cc(N(c3ccccc3)c3ccc4ccc5cc6oc7ccccc7c6cc5c4c3)ccc2C1(C)C ; c1ccc(-c2cccc(N(c3ccc4ccc5cc6oc7ccccc7c6cc5c4c3)c3cccc4ccccc34)c2)cc1]

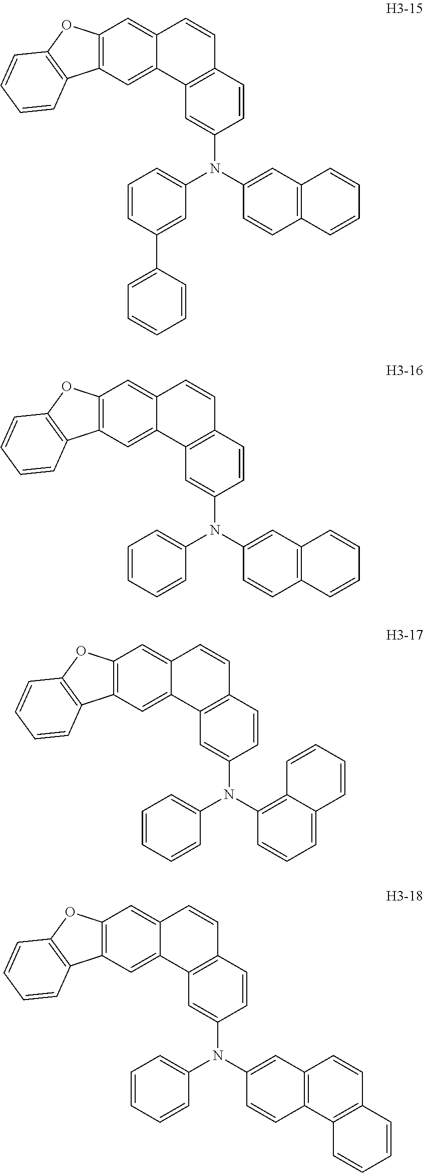 [SMILES: c1ccc(-c2cccc(N(c3ccc4ccccc4c3)c3ccc4ccc5cc6oc7ccccc7c6cc5c4c3)c2)cc1 ; c1ccc(N(c2ccc3ccccc3c2)c2ccc3ccc4cc5oc6ccccc6c5cc4c3c2)cc1 ; c1ccc(N(c2ccc3ccc4cc5oc6ccccc6c5cc4c3c2)c2cccc3ccccc23)cc1 ; c1ccc(N(c2ccc3c(ccc4ccccc43)c2)c2ccc3ccc4cc5oc6ccccc6c5cc4c3c2)cc1]

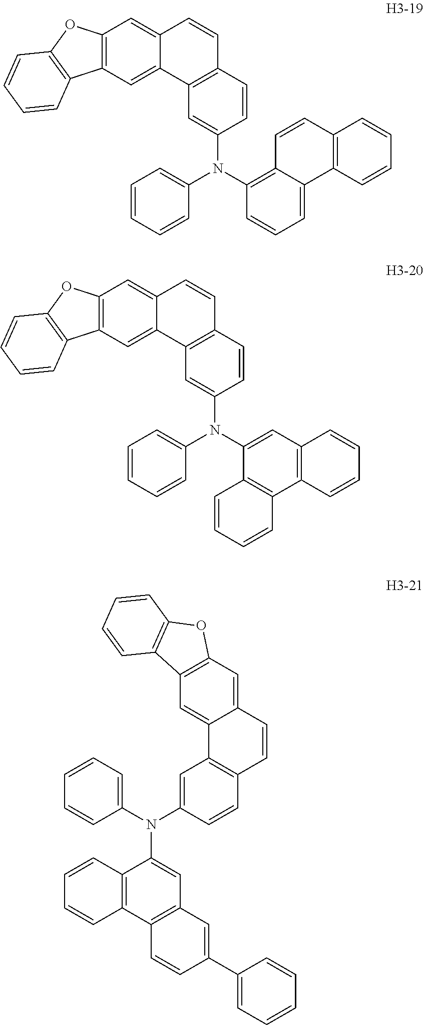 [SMILES: c1ccc(N(c2ccc3ccc4cc5oc6ccccc6c5cc4c3c2)c2cccc3c2ccc2ccccc23)cc1 ; c1ccc(N(c2ccc3ccc4cc5oc6ccccc6c5cc4c3c2)c2cc3ccccc3c3ccccc23)cc1 ; c1ccc(-c2ccc3c(c2)cc(N(c2ccccc2)c2ccc4ccc5cc6oc7ccccc7c6cc5c4c2)c2ccccc23)cc1]

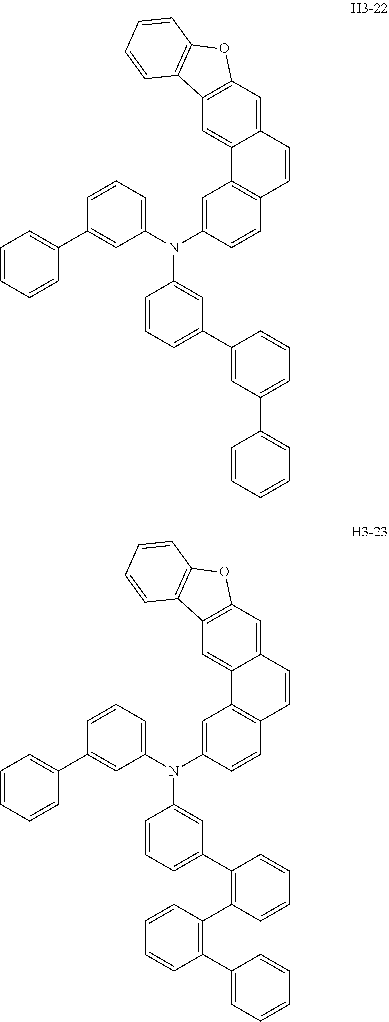 [SMILES: c1ccc(-c2cccc(-c3cccc(N(c4cccc(-c5ccccc5)c4)c4ccc5ccc6cc7oc8ccccc8c7cc6c5c4)c3)c2)cc1 ; c1ccc(-c2cccc(N(c3cccc(-c4ccccc4-c4ccccc4-c4ccccc4)c3)c3ccc4ccc5cc6oc7ccccc7c6cc5c4c3)c2)cc1]

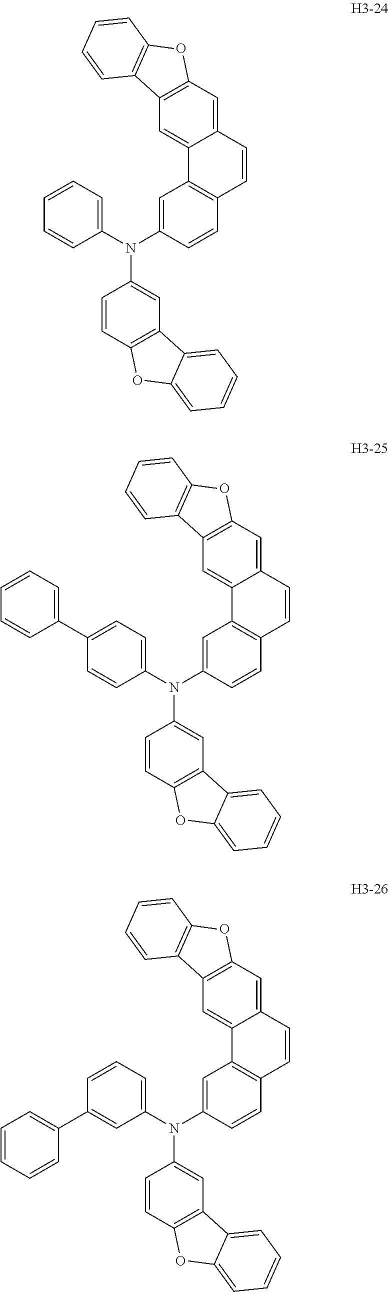 [SMILES: c1ccc(N(c2ccc3ccc4cc5oc6ccccc6c5cc4c3c2)c2ccc3oc4ccccc4c3c2)cc1 ; c1ccc(-c2ccc(N(c3ccc4ccc5cc6oc7ccccc7c6cc5c4c3)c3ccc4oc5ccccc5c4c3)cc2)cc1 ; c1ccc(-c2cccc(N(c3ccc4ccc5cc6oc7ccccc7c6cc5c4c3)c3ccc4oc5ccccc5c4c3)c2)cc1]

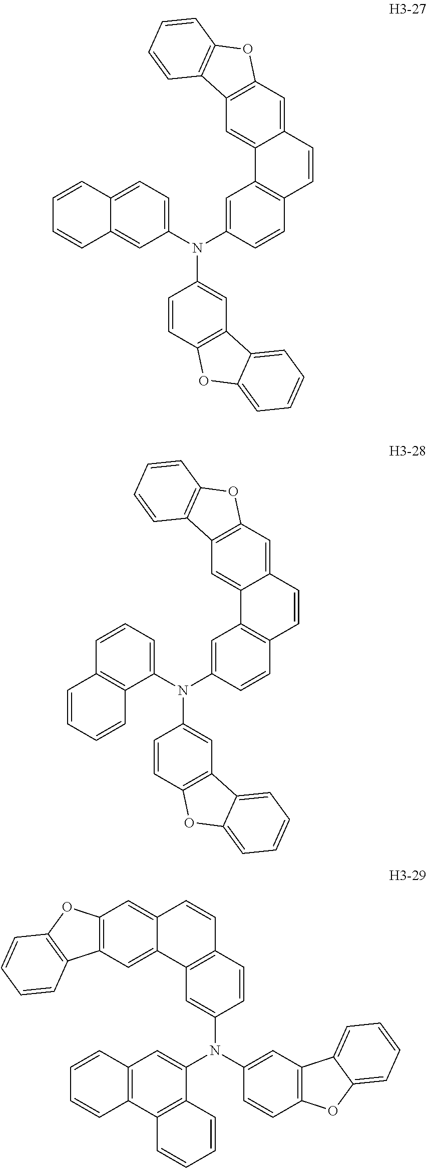 [SMILES: c1ccc2cc(N(c3ccc4ccc5cc6oc7ccccc7c6cc5c4c3)c3ccc4oc5ccccc5c4c3)ccc2c1 ; c1ccc2c(N(c3ccc4ccc5cc6oc7ccccc7c6cc5c4c3)c3ccc4oc5ccccc5c4c3)cccc2c1 ; c1ccc2c(c1)cc(N(c1ccc3ccc4cc5oc6ccccc6c5cc4c3c1)c1ccc3oc4ccccc4c3c1)c1ccccc12]

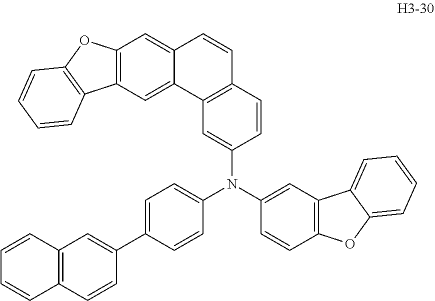 [SMILES: c1ccc2cc(-c3ccc(N(c4ccc5ccc6cc7oc8ccccc8c7cc6c5c4)c4ccc5oc6ccccc6c5c4)cc3)ccc2c1]

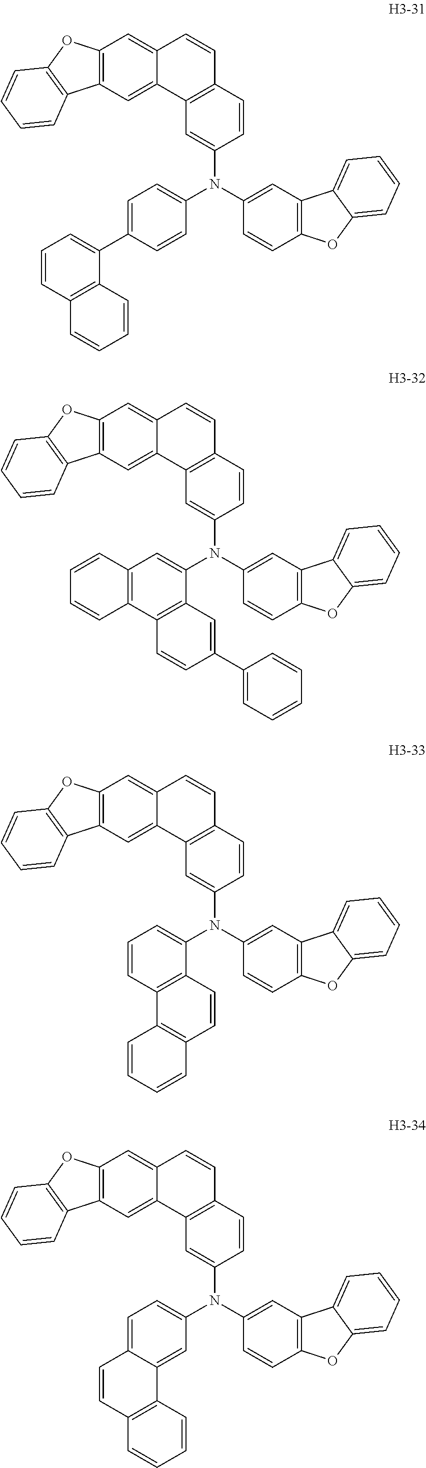 [SMILES: c1ccc2c(-c3ccc(N(c4ccc5ccc6cc7oc8ccccc8c7cc6c5c4)c4ccc5oc6ccccc6c5c4)cc3)cccc2c1 ; c1ccc(-c2ccc3c(c2)c(N(c2ccc4ccc5cc6oc7ccccc7c6cc5c4c2)c2ccc4oc5ccccc5c4c2)cc2ccccc23)cc1 ; c1ccc2c(c1)ccc1c(N(c3ccc4ccc5cc6oc7ccccc7c6cc5c4c3)c3ccc4oc5ccccc5c4c3)cccc12 ; c1ccc2c(c1)ccc1ccc(N(c3ccc4ccc5cc6oc7ccccc7c6cc5c4c3)c3ccc4oc5ccccc5c4c3)cc12]

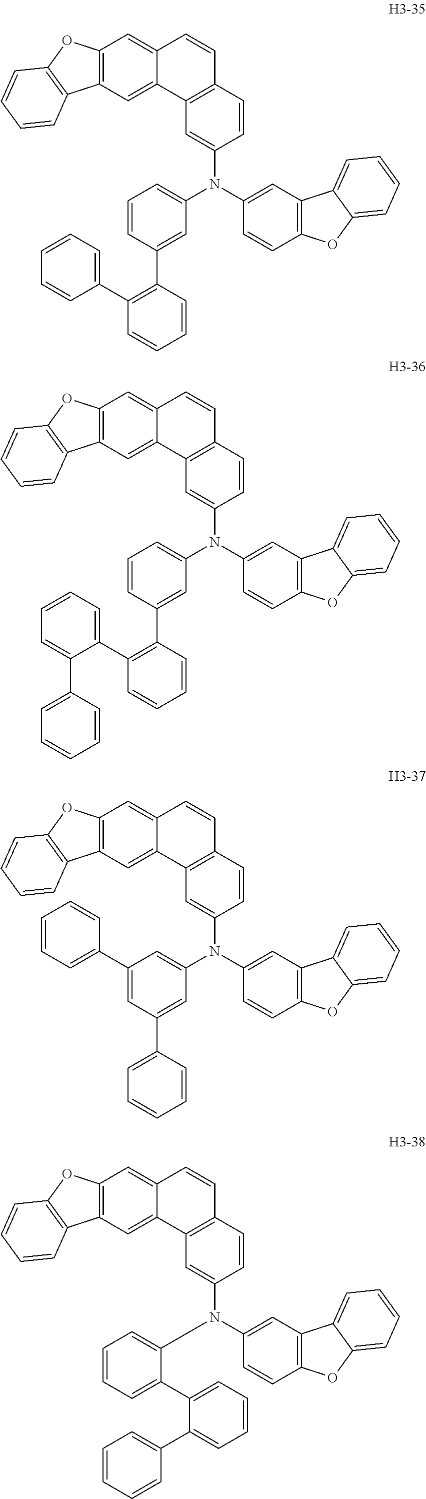 [SMILES: c1ccc(-c2ccccc2-c2cccc(N(c3ccc4ccc5cc6oc7ccccc7c6cc5c4c3)c3ccc4oc5ccccc5c4c3)c2)cc1 ; c1ccc(-c2ccccc2-c2ccccc2-c2cccc(N(c3ccc4ccc5cc6oc7ccccc7c6cc5c4c3)c3ccc4oc5ccccc5c4c3)c2)cc1 ; c1ccc(-c2cc(-c3ccccc3)cc(N(c3ccc4ccc5cc6oc7ccccc7c6cc5c4c3)c3ccc4oc5ccccc5c4c3)c2)cc1 ; c1ccc(-c2ccccc2-c2ccccc2N(c2ccc3ccc4cc5oc6ccccc6c5cc4c3c2)c2ccc3oc4ccccc4c3c2)cc1]

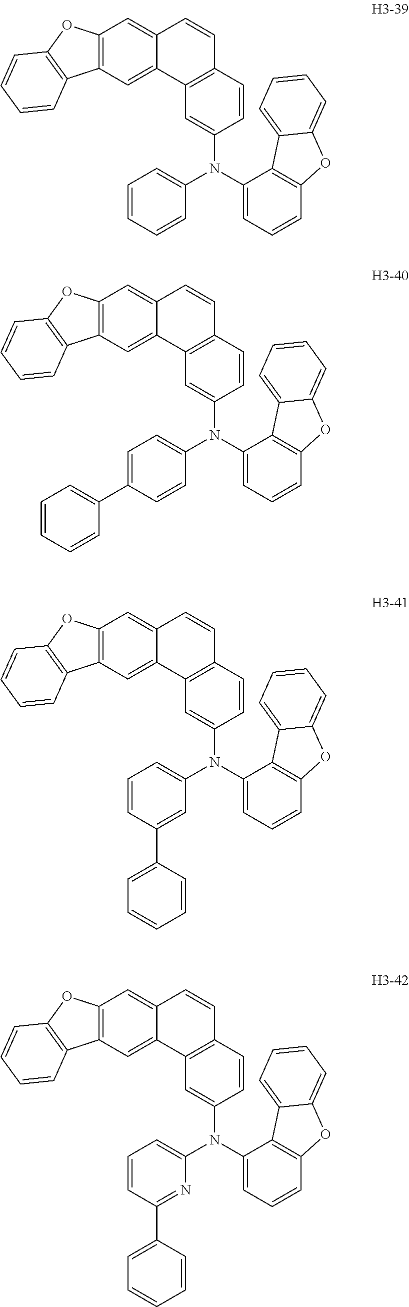 [SMILES: c1ccc(N(c2ccc3ccc4cc5oc6ccccc6c5cc4c3c2)c2cccc3oc4ccccc4c23)cc1 ; c1ccc(-c2ccc(N(c3ccc4ccc5cc6oc7ccccc7c6cc5c4c3)c3cccc4oc5ccccc5c34)cc2)cc1 ; c1ccc(-c2cccc(N(c3ccc4ccc5cc6oc7ccccc7c6cc5c4c3)c3cccc4oc5ccccc5c34)c2)cc1 ; c1ccc(-c2cccc(N(c3ccc4ccc5cc6oc7ccccc7c6cc5c4c3)c3cccc4oc5ccccc5c34)n2)cc1]

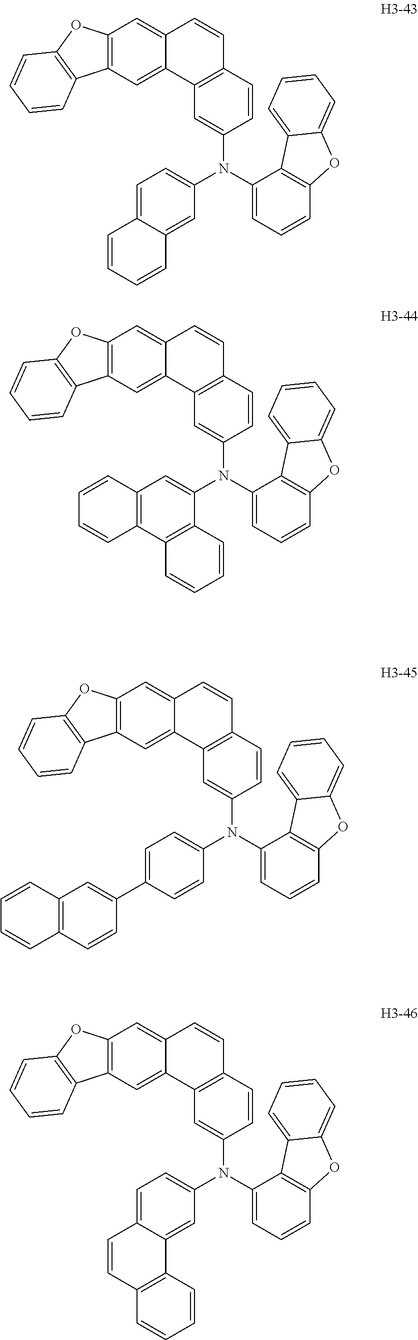

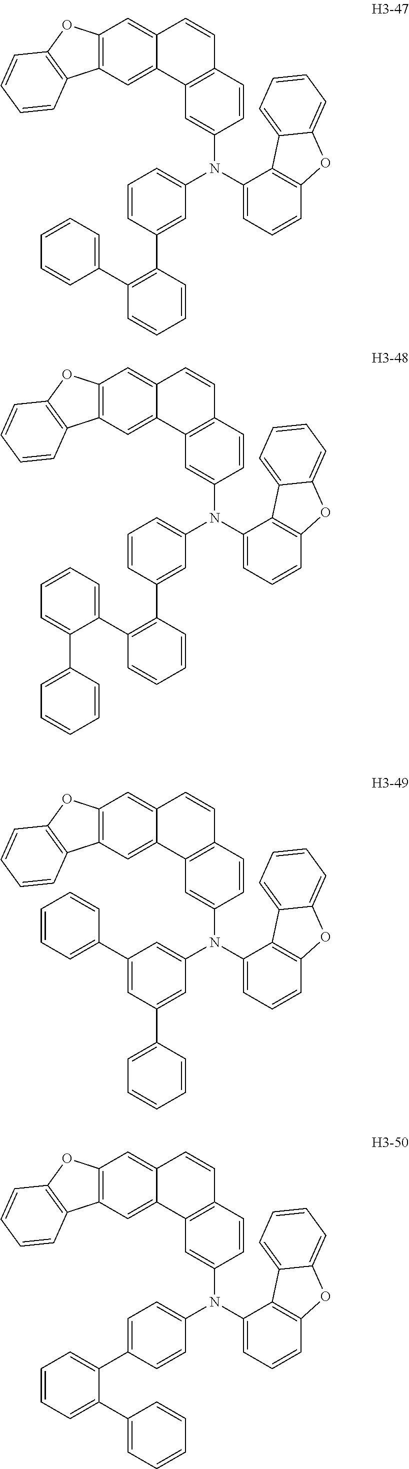

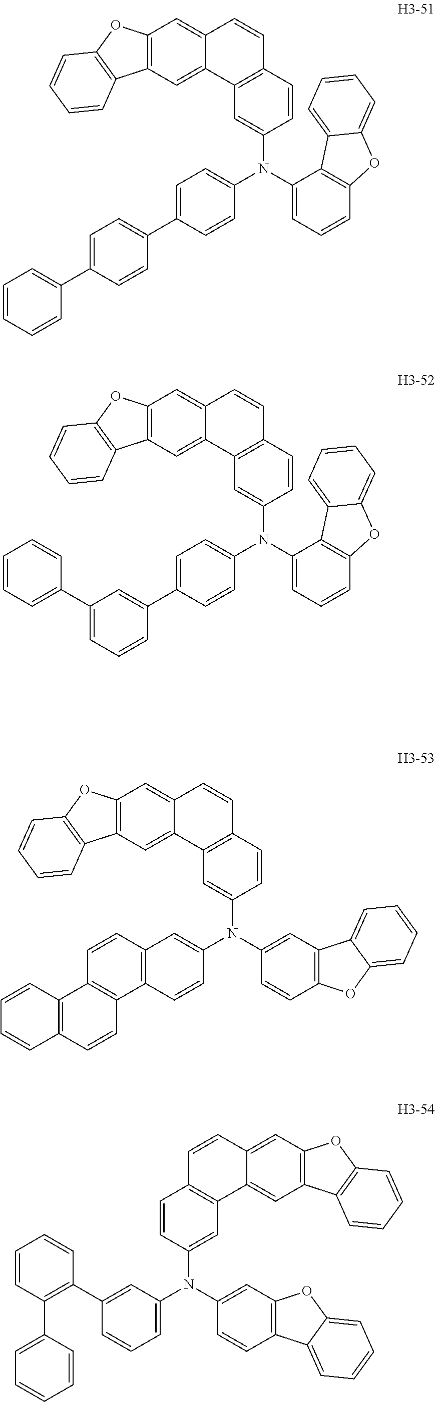 [SMILES: c1ccc(-c2ccc(-c3ccc(N(c4ccc5ccc6cc7oc8ccccc8c7cc6c5c4)c4cccc5oc6ccccc6c45)cc3)cc2)cc1 ; c1ccc(-c2cccc(-c3ccc(N(c4ccc5ccc6cc7oc8ccccc8c7cc6c5c4)c4cccc5oc6ccccc6c45)cc3)c2)cc1 ; c1ccc2c(c1)ccc1c3ccc(N(c4ccc5ccc6cc7oc8ccccc8c7cc6c5c4)c4ccc5oc6ccccc6c5c4)cc3ccc21 ; c1ccc(-c2ccccc2-c2cccc(N(c3ccc4c(c3)oc3ccccc34)c3ccc4ccc5cc6oc7ccccc7c6cc5c4c3)c2)cc1]

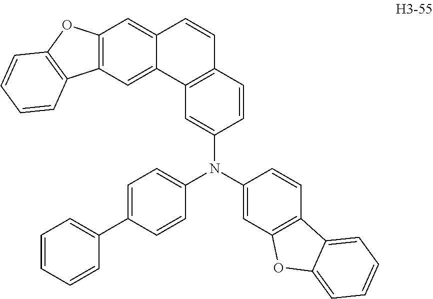 [SMILES: c1ccc(-c2ccc(N(c3ccc4c(c3)oc3ccccc34)c3ccc4ccc5cc6oc7ccccc7c6cc5c4c3)cc2)cc1]

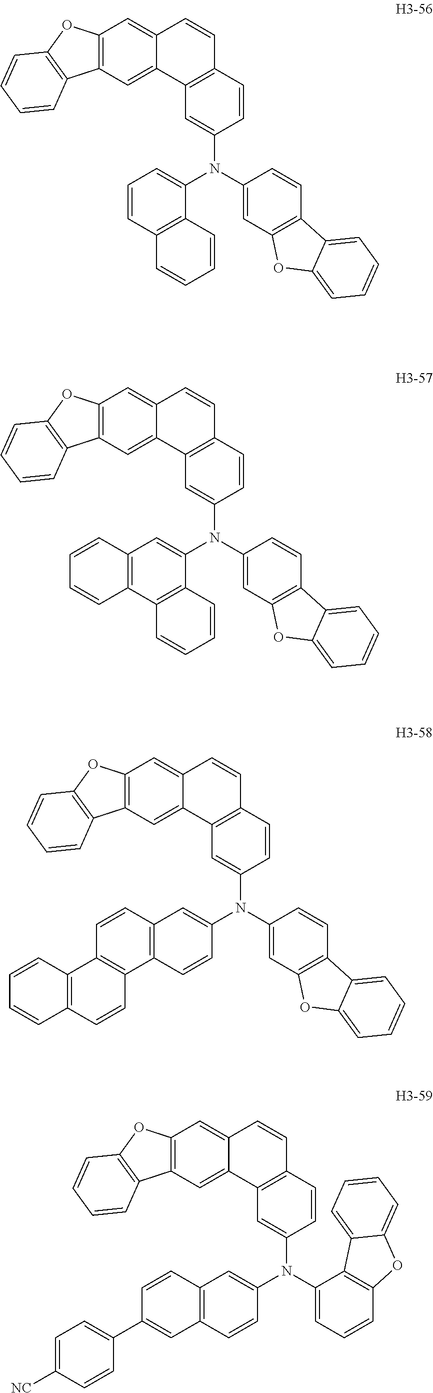 [SMILES: c1ccc2c(N(c3ccc4c(c3)oc3ccccc34)c3ccc4ccc5cc6oc7ccccc7c6cc5c4c3)cccc2c1 ; c1ccc2c(c1)cc(N(c1ccc3c(c1)oc1ccccc13)c1ccc3ccc4cc5oc6ccccc6c5cc4c3c1)c1ccccc12 ; c1ccc2c(c1)ccc1c3ccc(N(c4ccc5c(c4)oc4ccccc45)c4ccc5ccc6cc7oc8ccccc8c7cc6c5c4)cc3ccc21 ; N#Cc1ccc(-c2ccc3cc(N(c4ccc5ccc6cc7oc8ccccc8c7cc6c5c4)c4cccc5oc6ccccc6c45)ccc3c2)cc1]

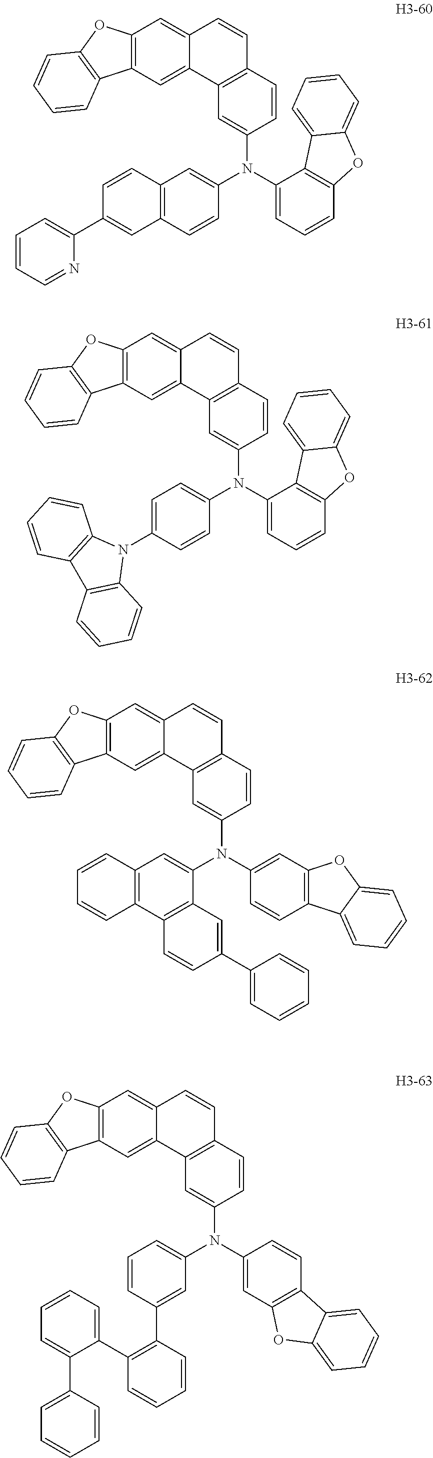 [SMILES: c1ccc(-c2ccc3cc(N(c4ccc5ccc6cc7oc8ccccc8c7cc6c5c4)c4cccc5oc6ccccc6c45)ccc3c2)nc1 ; c1ccc2c(c1)oc1cc3ccc4ccc(N(c5ccc(-n6c7ccccc7c7ccccc76)cc5)c5cccc6oc7ccccc7c56)cc4c3cc12 ; c1ccc(-c2ccc3c(c2)c(N(c2ccc4c(c2)oc2ccccc24)c2ccc4ccc5cc6oc7ccccc7c6cc5c4c2)cc2ccccc23)cc1 ; c1ccc(-c2ccccc2-c2ccccc2-c2cccc(N(c3ccc4c(c3)oc3ccccc34)c3ccc4ccc5cc6oc7ccccc7c6cc5c4c3)c2)cc1]

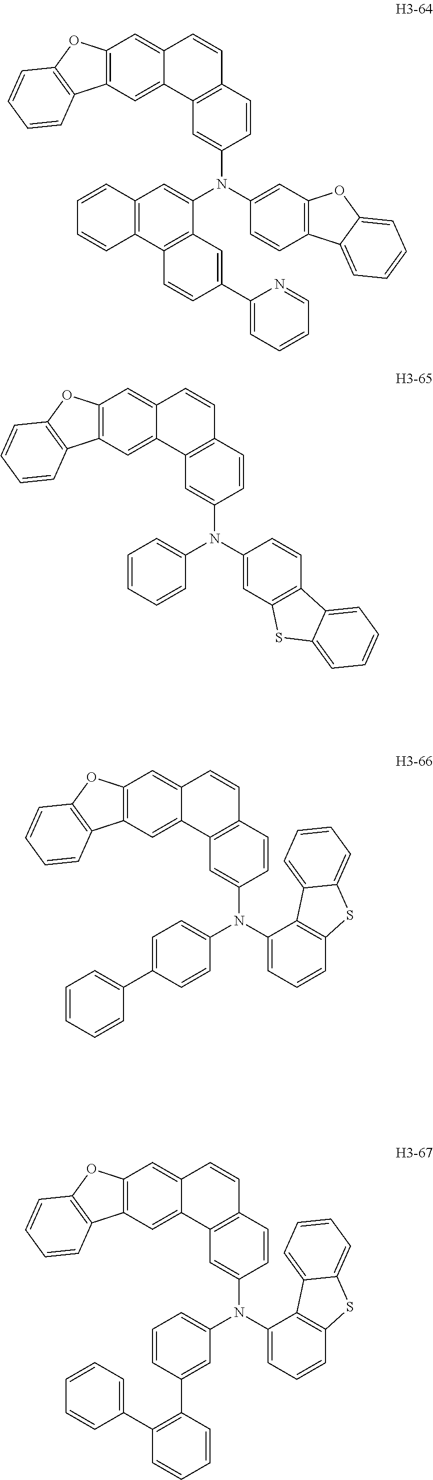 [SMILES: c1ccc(-c2ccc3c(c2)c(N(c2ccc4c(c2)oc2ccccc24)c2ccc4ccc5cc6oc7ccccc7c6cc5c4c2)cc2ccccc23)nc1 ; c1ccc(N(c2ccc3c(c2)sc2ccccc23)c2ccc3ccc4cc5oc6ccccc6c5cc4c3c2)cc1 ; c1ccc(-c2ccc(N(c3ccc4ccc5cc6oc7ccccc7c6cc5c4c3)c3cccc4sc5ccccc5c34)cc2)cc1 ; c1ccc(-c2ccccc2-c2cccc(N(c3ccc4ccc5cc6oc7ccccc7c6cc5c4c3)c3cccc4sc5ccccc5c34)c2)cc1]

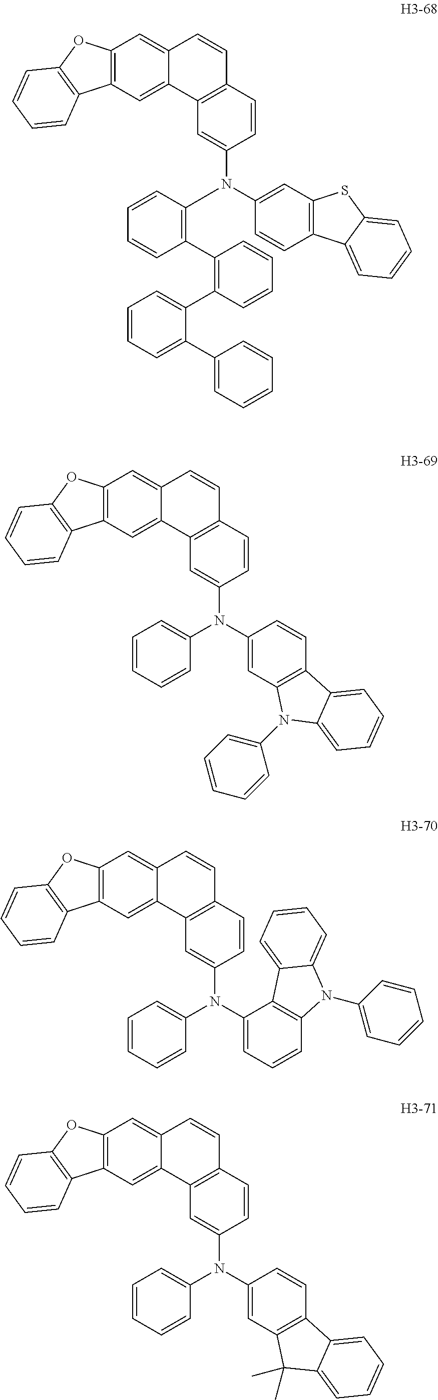 [SMILES: c1ccc(-c2ccccc2-c2ccccc2-c2ccccc2N(c2ccc3c(c2)sc2ccccc23)c2ccc3ccc4cc5oc6ccccc6c5cc4c3c2)cc1 ; c1ccc(N(c2ccc3ccc4cc5oc6ccccc6c5cc4c3c2)c2ccc3c4ccccc4n(-c4ccccc4)c3c2)cc1 ; c1ccc(N(c2ccc3ccc4cc5oc6ccccc6c5cc4c3c2)c2cccc3c2c2ccccc2n3-c2ccccc2)cc1 ; CC1(C)c2ccccc2-c2ccc(N(c3ccccc3)c3ccc4ccc5cc6oc7ccccc7c6cc5c4c3)cc21]

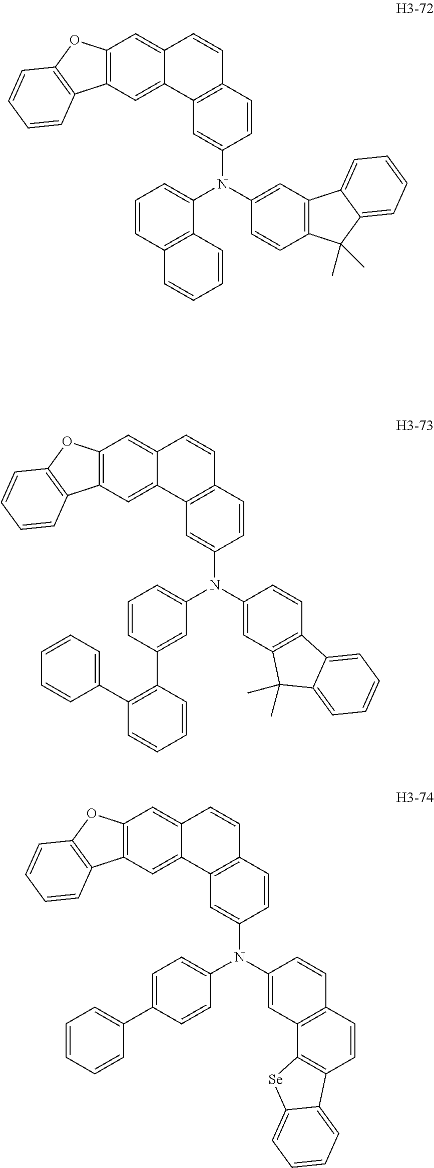 [SMILES: CC1(C)c2ccccc2-c2cc(N(c3ccc4ccc5cc6oc7ccccc7c6cc5c4c3)c3cccc4ccccc34)ccc21 ; CC1(C)c2ccccc2-c2ccc(N(c3cccc(-c4ccccc4-c4ccccc4)c3)c3ccc4ccc5cc6oc7ccccc7c6cc5c4c3)cc21 ; c1ccc(-c2ccc(N(c3ccc4ccc5cc6oc7ccccc7c6cc5c4c3)c3ccc4ccc5c6ccccc6[se]c5c4c3)cc2)cc1]

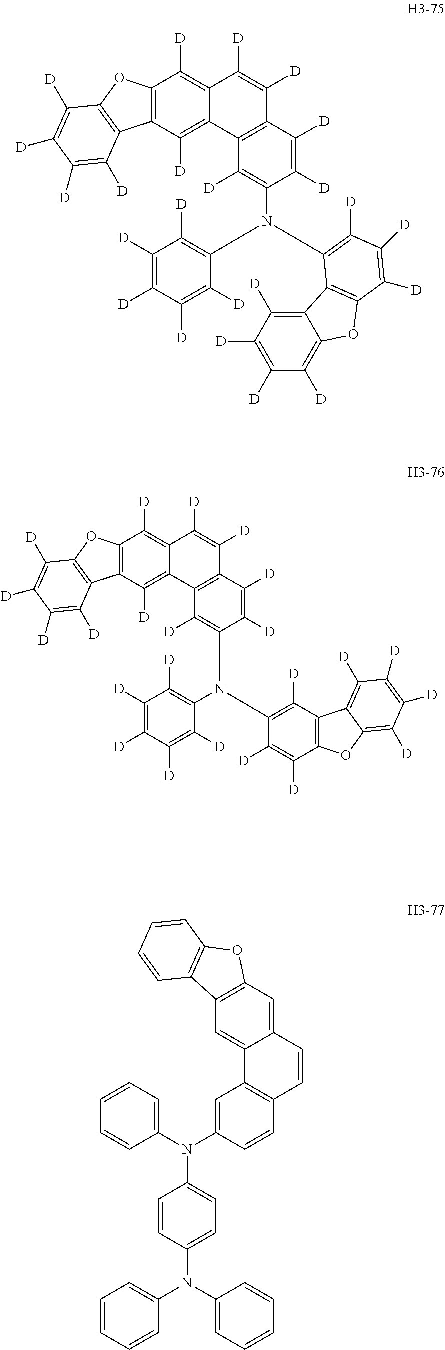 [SMILES: [2H]c1c([2H])c([2H])c(N(c2c([2H])c([2H])c3c([2H])c([2H])c4c([2H])c5oc6c([2H])c([2H])c([2H])c([2H])c6c5c([2H])c4c3c2[2H])c2c([2H])c([2H])c([2H])c3oc4c([2H])c([2H])c([2H])c([2H])c4c23)c([2H])c1[2H] ; [2H]c1c([2H])c([2H])c(N(c2c([2H])c([2H])c3oc4c([2H])c([2H])c([2H])c([2H])c4c3c2[2H])c2c([2H])c([2H])c3c([2H])c([2H])c4c([2H])c5oc6c([2H])c([2H])c([2H])c([2H])c6c5c([2H])c4c3c2[2H])c([2H])c1[2H] ; c1ccc(N(c2ccccc2)c2ccc(N(c3ccccc3)c3ccc4ccc5cc6oc7ccccc7c6cc5c4c3)cc2)cc1]

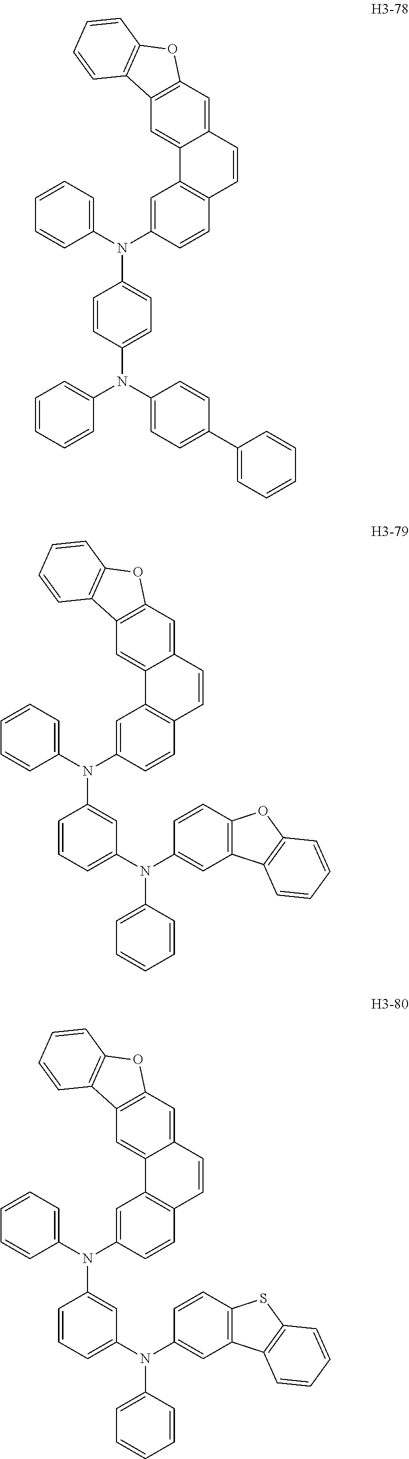 [SMILES: c1ccc(-c2ccc(N(c3ccccc3)c3ccc(N(c4ccccc4)c4ccc5ccc6cc7oc8ccccc8c7cc6c5c4)cc3)cc2)cc1 ; c1ccc(N(c2cccc(N(c3ccccc3)c3ccc4oc5ccccc5c4c3)c2)c2ccc3ccc4cc5oc6ccccc6c5cc4c3c2)cc1 ; c1ccc(N(c2cccc(N(c3ccccc3)c3ccc4sc5ccccc5c4c3)c2)c2ccc3ccc4cc5oc6ccccc6c5cc4c3c2)cc1]

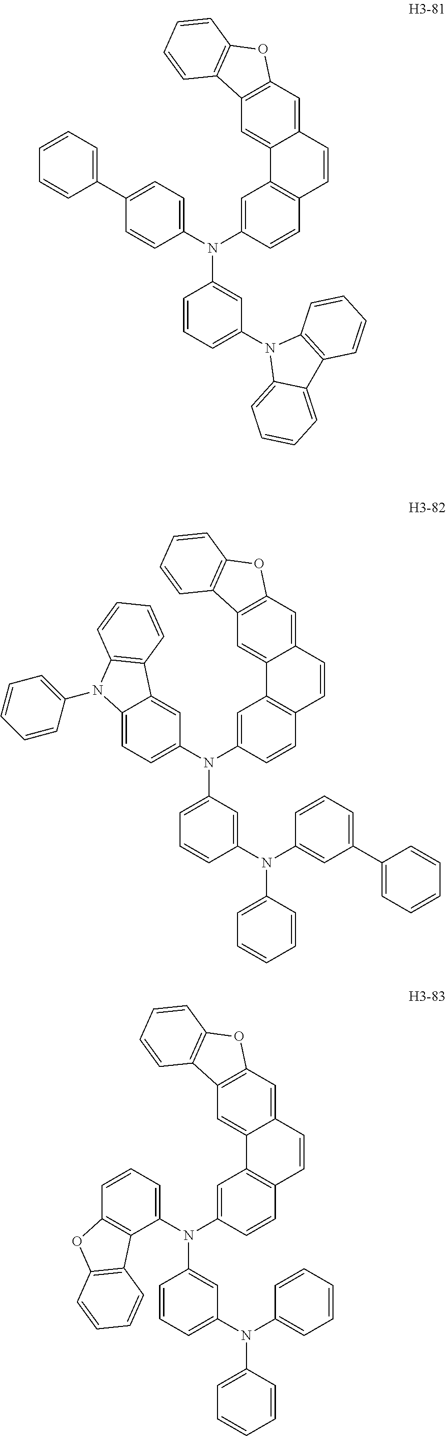 [SMILES: c1ccc(-c2ccc(N(c3cccc(-n4c5ccccc5c5ccccc54)c3)c3ccc4ccc5cc6oc7ccccc7c6cc5c4c3)cc2)cc1 ; c1ccc(-c2cccc(N(c3ccccc3)c3cccc(N(c4ccc5ccc6cc7oc8ccccc8c7cc6c5c4)c4ccc5c(c4)c4ccccc4n5-c4ccccc4)c3)c2)cc1 ; c1ccc(N(c2ccccc2)c2cccc(N(c3ccc4ccc5cc6oc7ccccc7c6cc5c4c3)c3cccc4oc5ccccc5c34)c2)cc1]

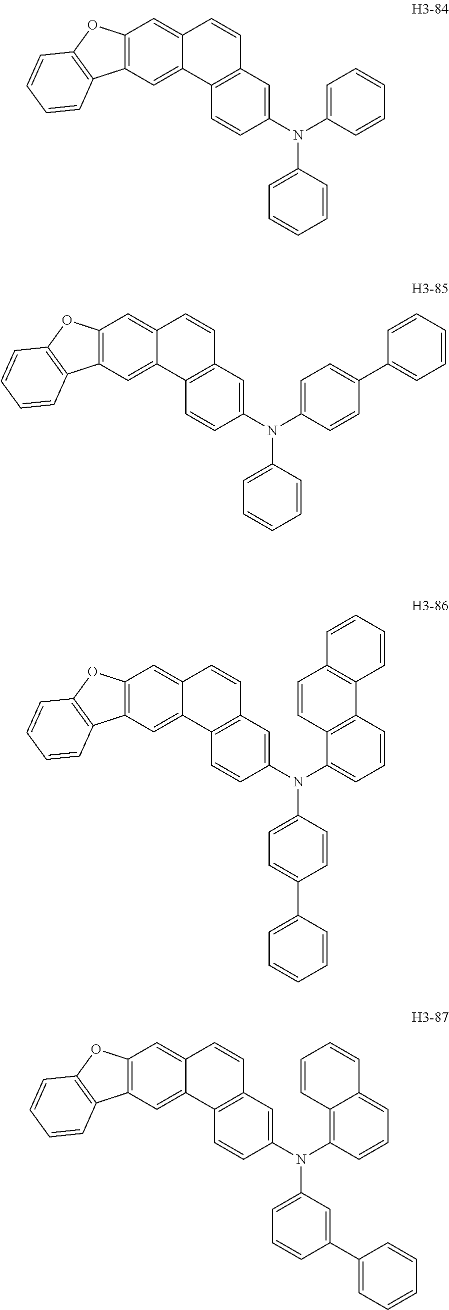 [SMILES: c1ccc(N(c2ccccc2)c2ccc3c(ccc4cc5oc6ccccc6c5cc43)c2)cc1 ; c1ccc(-c2ccc(N(c3ccccc3)c3ccc4c(ccc5cc6oc7ccccc7c6cc54)c3)cc2)cc1 ; c1ccc(-c2ccc(N(c3ccc4c(ccc5cc6oc7ccccc7c6cc54)c3)c3cccc4c3ccc3ccccc34)cc2)cc1 ; c1ccc(-c2cccc(N(c3ccc4c(ccc5cc6oc7ccccc7c6cc54)c3)c3cccc4ccccc34)c2)cc1]

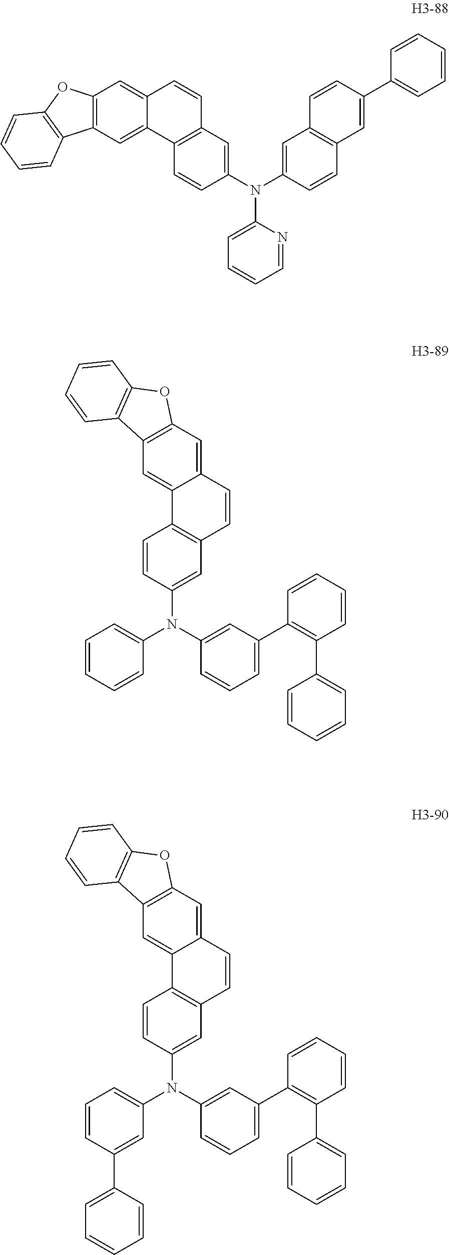 [SMILES: c1ccc(-c2ccc3cc(N(c4ccc5c(ccc6cc7oc8ccccc8c7cc65)c4)c4ccccn4)ccc3c2)cc1 ; c1ccc(-c2ccccc2-c2cccc(N(c3ccccc3)c3ccc4c(ccc5cc6oc7ccccc7c6cc54)c3)c2)cc1 ; c1ccc(-c2cccc(N(c3cccc(-c4ccccc4-c4ccccc4)c3)c3ccc4c(ccc5cc6oc7ccccc7c6cc54)c3)c2)cc1]

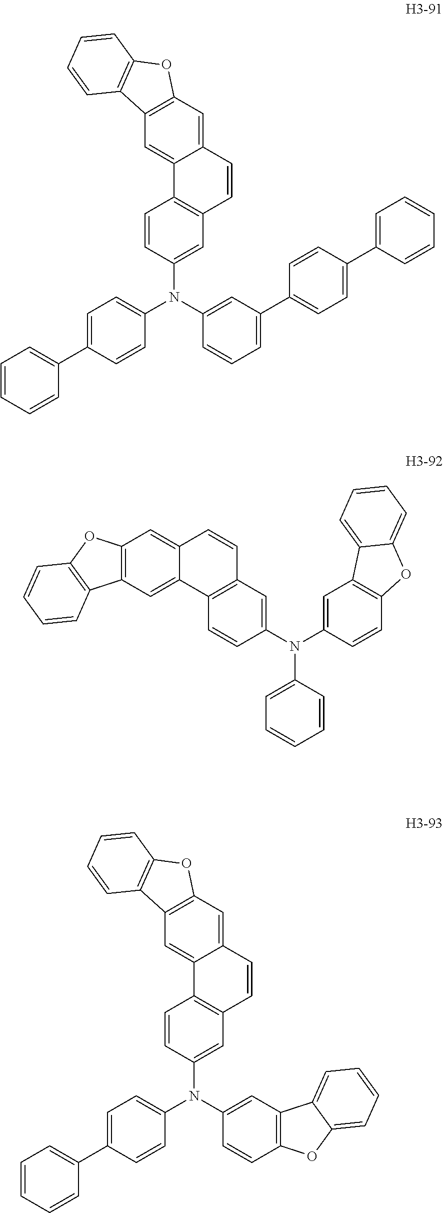 [SMILES: c1ccc(-c2ccc(-c3cccc(N(c4ccc(-c5ccccc5)cc4)c4ccc5c(ccc6cc7oc8ccccc8c7cc65)c4)c3)cc2)cc1 ; c1ccc(N(c2ccc3c(ccc4cc5oc6ccccc6c5cc43)c2)c2ccc3oc4ccccc4c3c2)cc1 ; c1ccc(-c2ccc(N(c3ccc4c(ccc5cc6oc7ccccc7c6cc54)c3)c3ccc4oc5ccccc5c4c3)cc2)cc1]

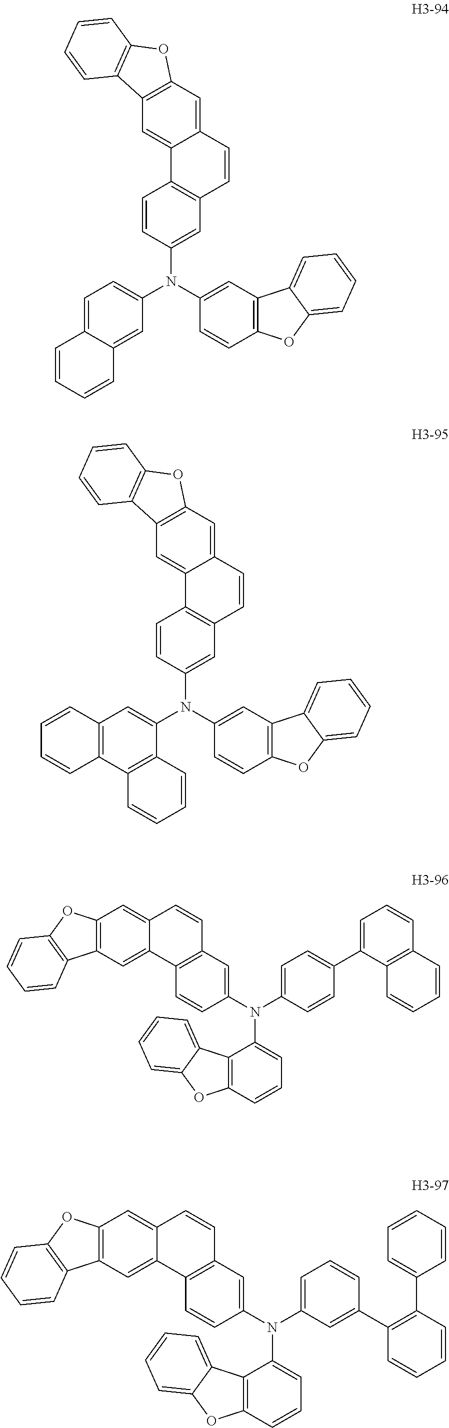 [SMILES: c1ccc2cc(N(c3ccc4c(ccc5cc6oc7ccccc7c6cc54)c3)c3ccc4oc5ccccc5c4c3)ccc2c1 ; c1ccc2c(c1)cc(N(c1ccc3c(ccc4cc5oc6ccccc6c5cc43)c1)c1ccc3oc4ccccc4c3c1)c1ccccc12 ; c1ccc2c(-c3ccc(N(c4ccc5c(ccc6cc7oc8ccccc8c7cc65)c4)c4cccc5oc6ccccc6c45)cc3)cccc2c1 ; c1ccc(-c2ccccc2-c2cccc(N(c3ccc4c(ccc5cc6oc7ccccc7c6cc54)c3)c3cccc4oc5ccccc5c34)c2)cc1]

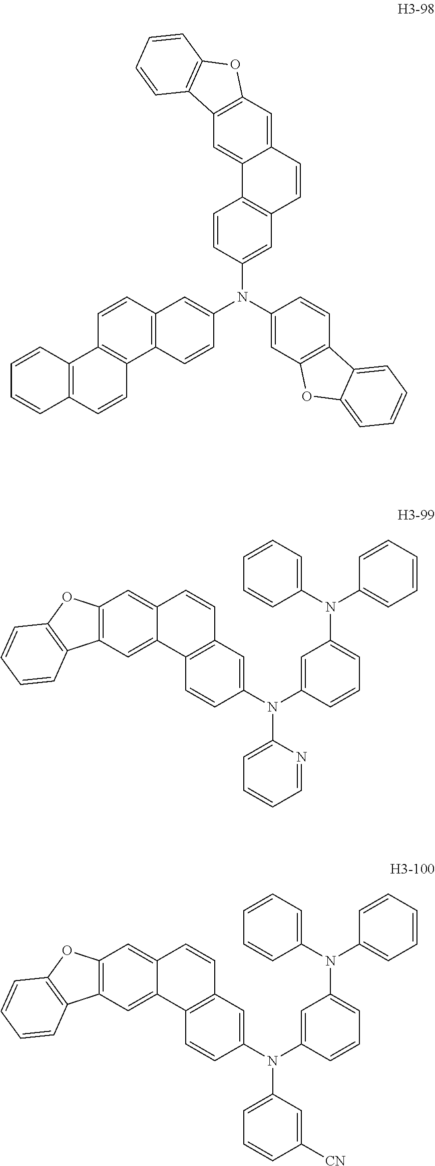 [SMILES: c1ccc2c(c1)ccc1c3ccc(N(c4ccc5c(ccc6cc7oc8ccccc8c7cc65)c4)c4ccc5c(c4)oc4ccccc45)cc3ccc21 ; c1ccc(N(c2ccccc2)c2cccc(N(c3ccc4c(ccc5cc6oc7ccccc7c6cc54)c3)c3ccccn3)c2)cc1 ; N#Cc1cccc(N(c2cccc(N(c3ccccc3)c3ccccc3)c2)c2ccc3c(ccc4cc5oc6ccccc6c5cc43)c2)c1]

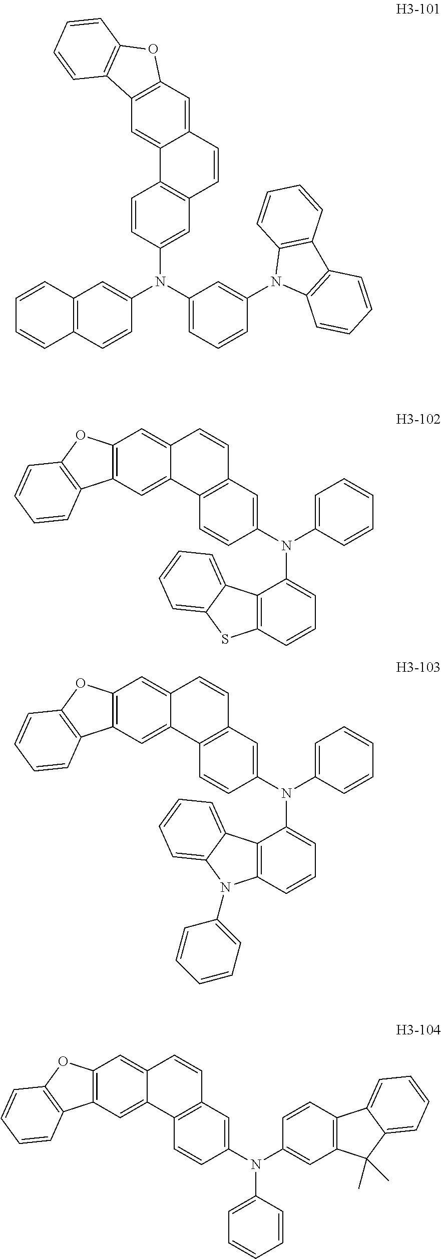

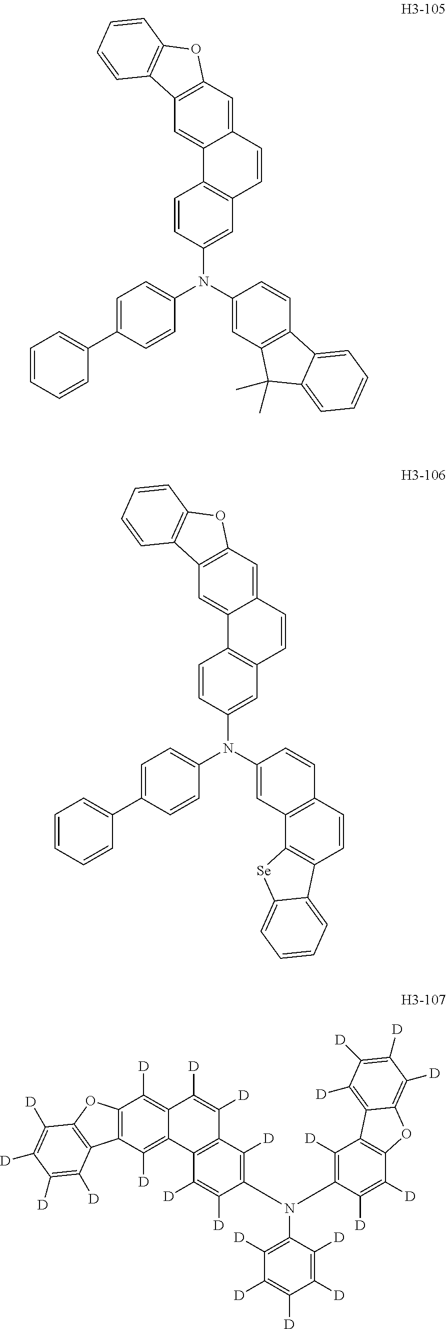 [SMILES: CC1(C)c2ccccc2-c2ccc(N(c3ccc(-c4ccccc4)cc3)c3ccc4c(ccc5cc6oc7ccccc7c6cc54)c3)cc21 ; c1ccc(-c2ccc(N(c3ccc4c(ccc5cc6oc7ccccc7c6cc54)c3)c3ccc4ccc5c6ccccc6[se]c5c4c3)cc2)cc1 ; [2H]c1c([2H])c([2H])c(N(c2c([2H])c([2H])c3c(c2[2H])c([2H])c([2H])c2c([2H])c4oc5c([2H])c([2H])c([2H])c([2H])c5c4c([2H])c23)c2c([2H])c([2H])c3oc4c([2H])c([2H])c([2H])c([2H])c4c3c2[2H])c([2H])c1[2H]]

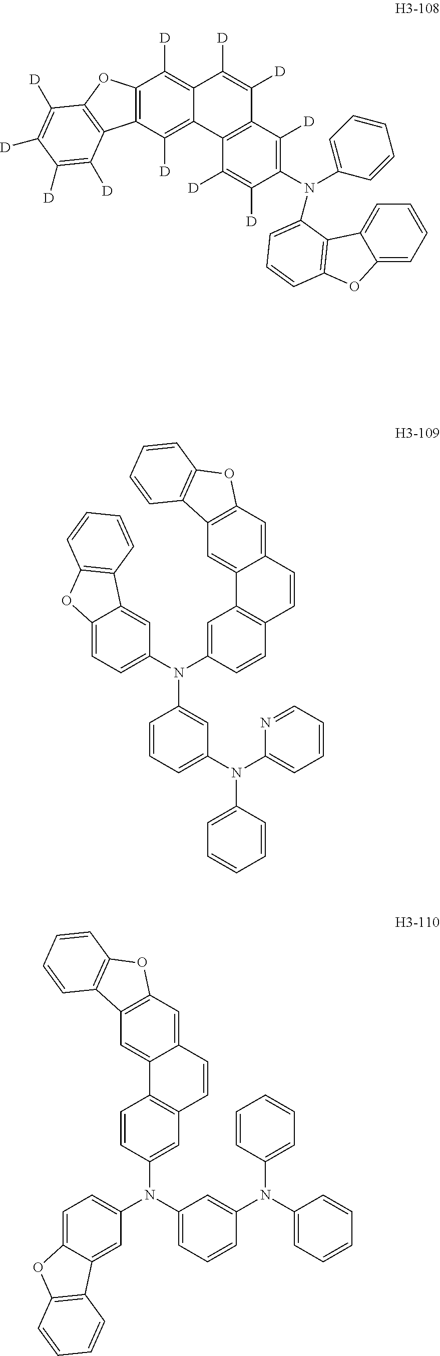 [SMILES: [2H]c1c([2H])c([2H])c2c(oc3c([2H])c4c([2H])c([2H])c5c([2H])c(N(c6ccccc6)c6cccc7oc8ccccc8c67)c([2H])c([2H])c5c4c([2H])c32)c1[2H] ; c1ccc(N(c2cccc(N(c3ccc4ccc5cc6oc7ccccc7c6cc5c4c3)c3ccc4oc5ccccc5c4c3)c2)c2ccccn2)cc1 ; c1ccc(N(c2ccccc2)c2cccc(N(c3ccc4c(ccc5cc6oc7ccccc7c6cc54)c3)c3ccc4oc5ccccc5c4c3)c2)cc1]

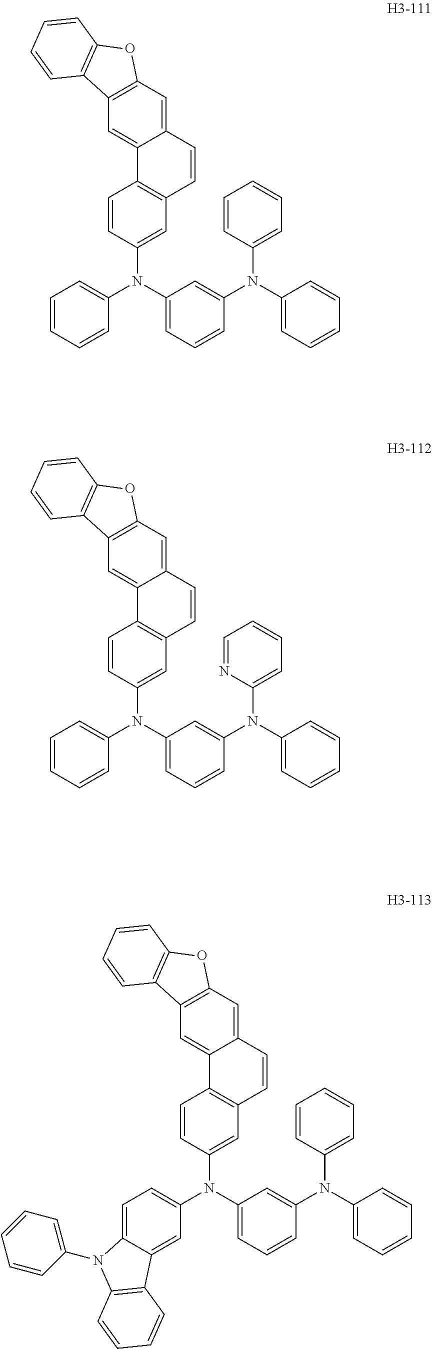 [SMILES: c1ccc(N(c2ccccc2)c2cccc(N(c3ccccc3)c3ccc4c(ccc5cc6oc7ccccc7c6cc54)c3)c2)cc1 ; c1ccc(N(c2cccc(N(c3ccccc3)c3ccccn3)c2)c2ccc3c(ccc4cc5oc6ccccc6c5cc43)c2)cc1 ; c1ccc(N(c2ccccc2)c2cccc(N(c3ccc4c(ccc5cc6oc7ccccc7c6cc54)c3)c3ccc4c(c3)c3ccccc3n4-c3ccccc3)c2)cc1]

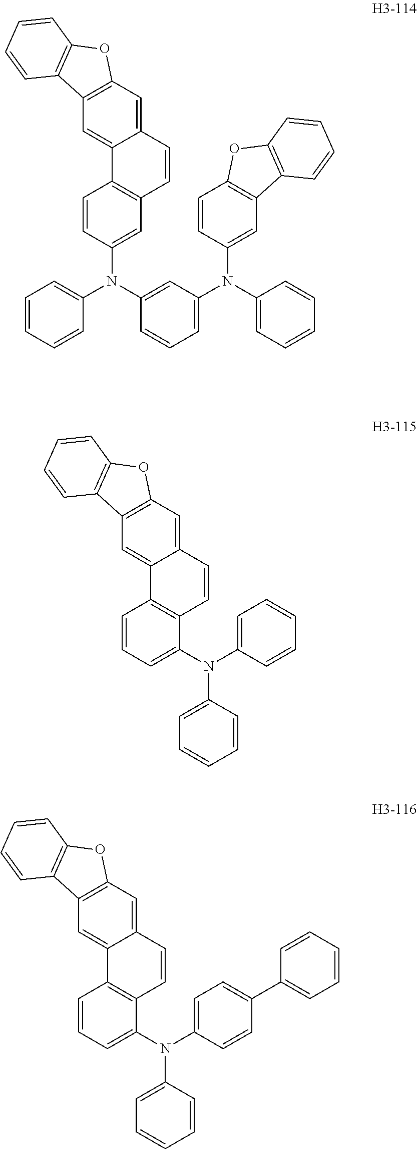 [SMILES: c1ccc(N(c2cccc(N(c3ccccc3)c3ccc4oc5ccccc5c4c3)c2)c2ccc3c(ccc4cc5oc6ccccc6c5cc43)c2)cc1 ; c1ccc(N(c2ccccc2)c2cccc3c2ccc2cc4oc5ccccc5c4cc23)cc1 ; c1ccc(-c2ccc(N(c3ccccc3)c3cccc4c3ccc3cc5oc6ccccc6c5cc34)cc2)cc1]

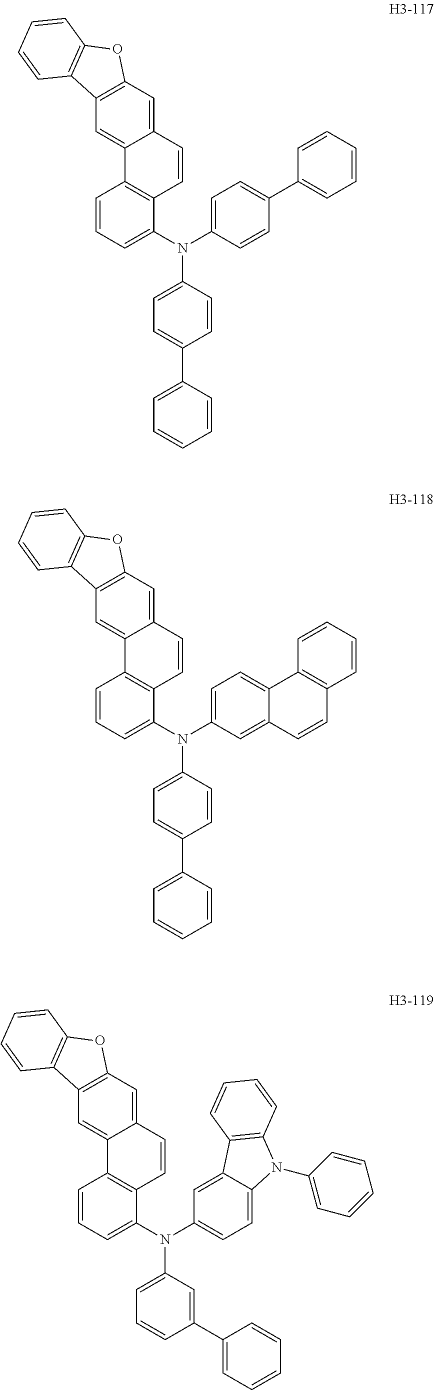 [SMILES: c1ccc(-c2ccc(N(c3ccc(-c4ccccc4)cc3)c3cccc4c3ccc3cc5oc6ccccc6c5cc34)cc2)cc1 ; c1ccc(-c2ccc(N(c3ccc4c(ccc5ccccc54)c3)c3cccc4c3ccc3cc5oc6ccccc6c5cc34)cc2)cc1 ; c1ccc(-c2cccc(N(c3ccc4c(c3)c3ccccc3n4-c3ccccc3)c3cccc4c3ccc3cc5oc6ccccc6c5cc34)c2)cc1]

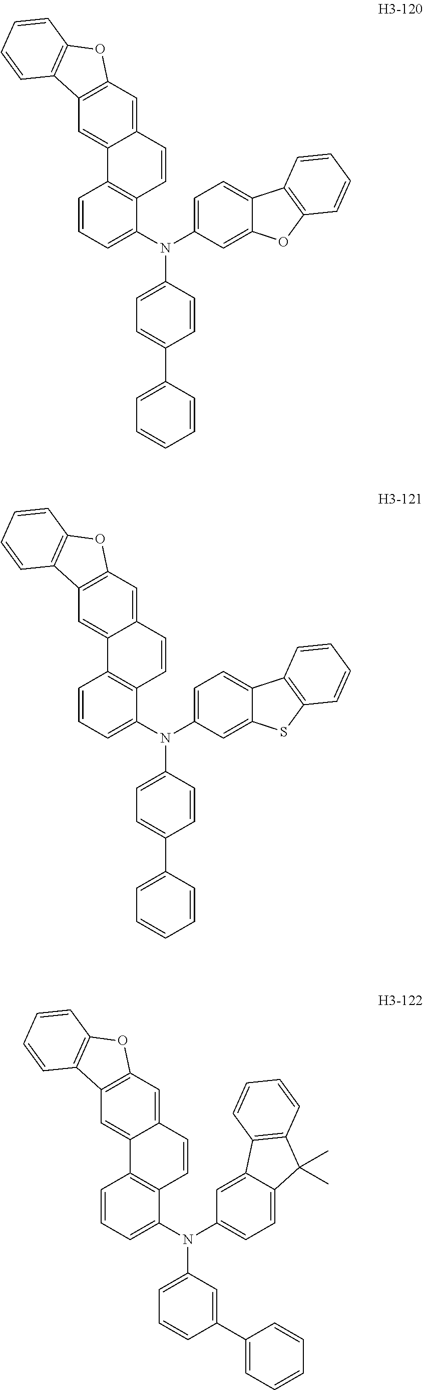 [SMILES: c1ccc(-c2ccc(N(c3ccc4c(c3)oc3ccccc34)c3cccc4c3ccc3cc5oc6ccccc6c5cc34)cc2)cc1 ; c1ccc(-c2ccc(N(c3ccc4c(c3)sc3ccccc34)c3cccc4c3ccc3cc5oc6ccccc6c5cc34)cc2)cc1 ; CC1(C)c2ccccc2-c2cc(N(c3cccc(-c4ccccc4)c3)c3cccc4c3ccc3cc5oc6ccccc6c5cc34)ccc21]

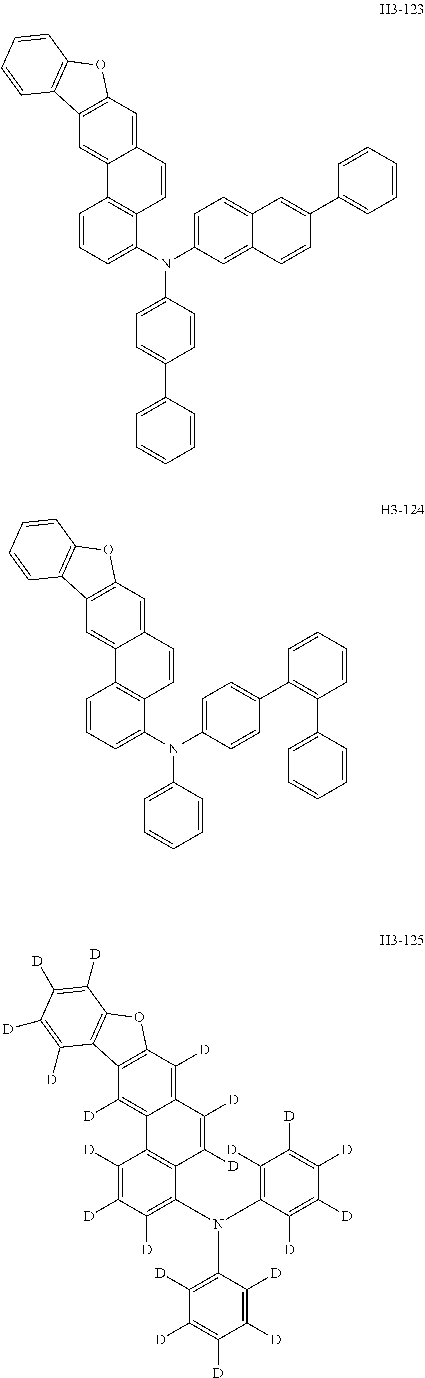 [SMILES: c1ccc(-c2ccc(N(c3ccc4cc(-c5ccccc5)ccc4c3)c3cccc4c3ccc3cc5oc6ccccc6c5cc34)cc2)cc1 ; c1ccc(-c2ccccc2-c2ccc(N(c3ccccc3)c3cccc4c3ccc3cc5oc6ccccc6c5cc34)cc2)cc1 ; [2H]c1c([2H])c([2H])c(N(c2c([2H])c([2H])c([2H])c([2H])c2[2H])c2c([2H])c([2H])c([2H])c3c2c([2H])c([2H])c2c([2H])c4oc5c([2H])c([2H])c([2H])c([2H])c5c4c([2H])c23)c([2H])c1[2H]]

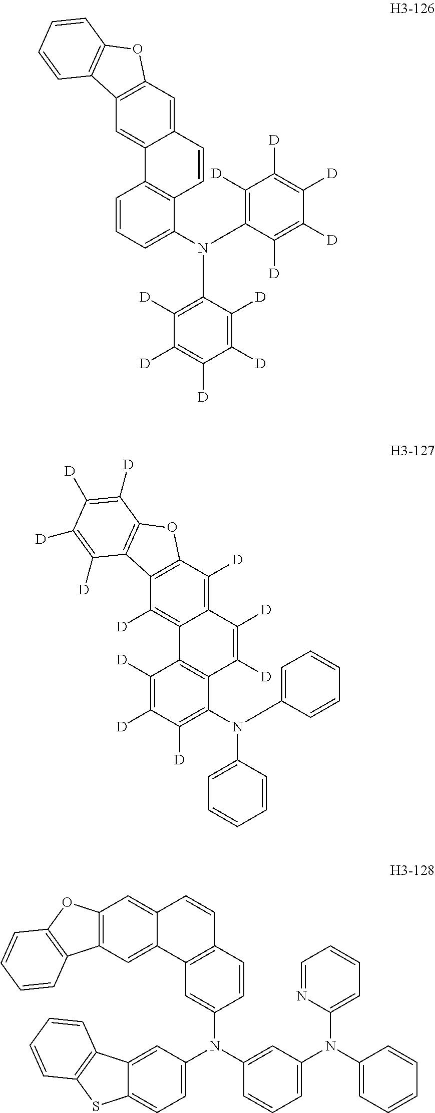 [SMILES: [2H]c1c([2H])c([2H])c(N(c2c([2H])c([2H])c([2H])c([2H])c2[2H])c2cccc3c2ccc2cc4oc5ccccc5c4cc23)c([2H])c1[2H] ; [2H]c1c([2H])c([2H])c2c(oc3c([2H])c4c([2H])c([2H])c5c(N(c6ccccc6)c6ccccc6)c([2H])c([2H])c([2H])c5c4c([2H])c32)c1[2H] ; c1ccc(N(c2cccc(N(c3ccc4ccc5cc6oc7ccccc7c6cc5c4c3)c3ccc4sc5ccccc5c4c3)c2)c2ccccn2)cc1]

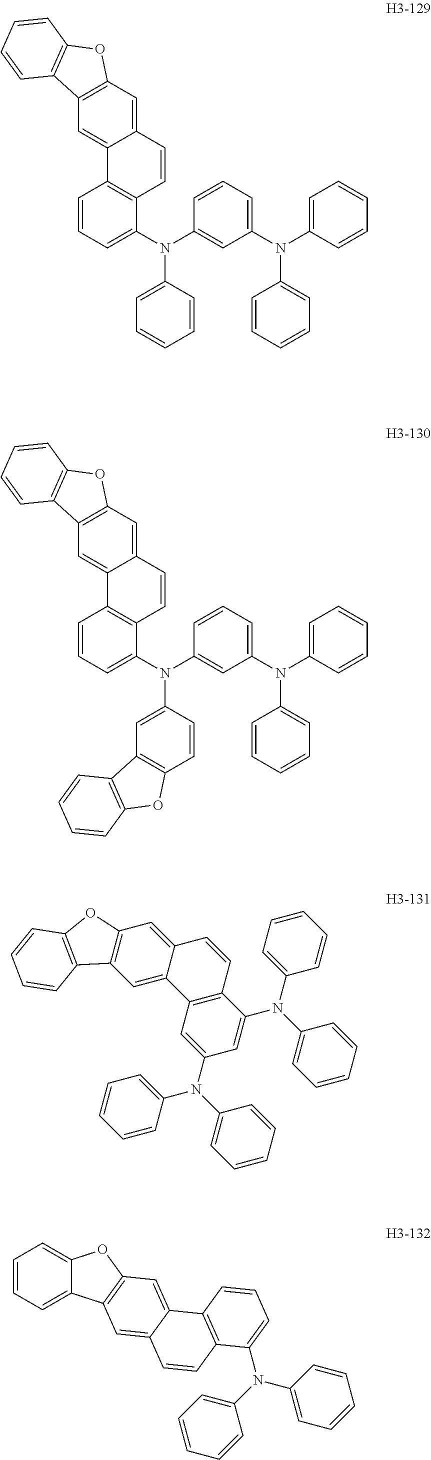 [SMILES: c1ccc(N(c2ccccc2)c2cccc(N(c3ccccc3)c3cccc4c3ccc3cc5oc6ccccc6c5cc34)c2)cc1 ; c1ccc(N(c2ccccc2)c2cccc(N(c3ccc4oc5ccccc5c4c3)c3cccc4c3ccc3cc5oc6ccccc6c5cc34)c2)cc1 ; c1ccc(N(c2ccccc2)c2cc(N(c3ccccc3)c3ccccc3)c3ccc4cc5oc6ccccc6c5cc4c3c2)cc1 ; c1ccc(N(c2ccccc2)c2cccc3c2ccc2cc4c(cc23)oc2ccccc24)cc1]

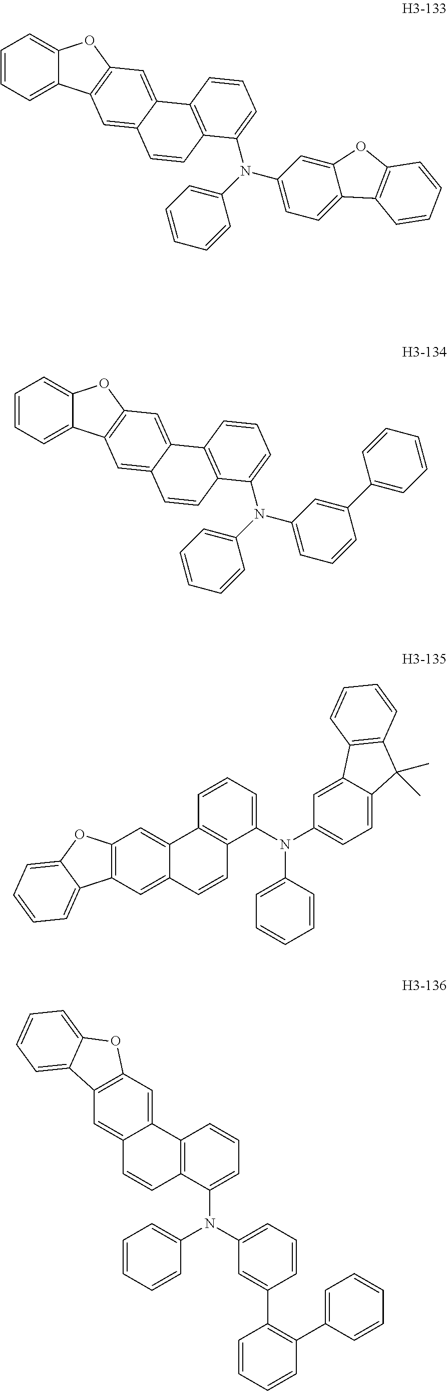 [SMILES: c1ccc(N(c2ccc3c(c2)oc2ccccc23)c2cccc3c2ccc2cc4c(cc23)oc2ccccc24)cc1 ; c1ccc(-c2cccc(N(c3ccccc3)c3cccc4c3ccc3cc5c(cc34)oc3ccccc35)c2)cc1 ; CC1(C)c2ccccc2-c2cc(N(c3ccccc3)c3cccc4c3ccc3cc5c(cc34)oc3ccccc35)ccc21 ; c1ccc(-c2ccccc2-c2cccc(N(c3ccccc3)c3cccc4c3ccc3cc5c(cc34)oc3ccccc35)c2)cc1]

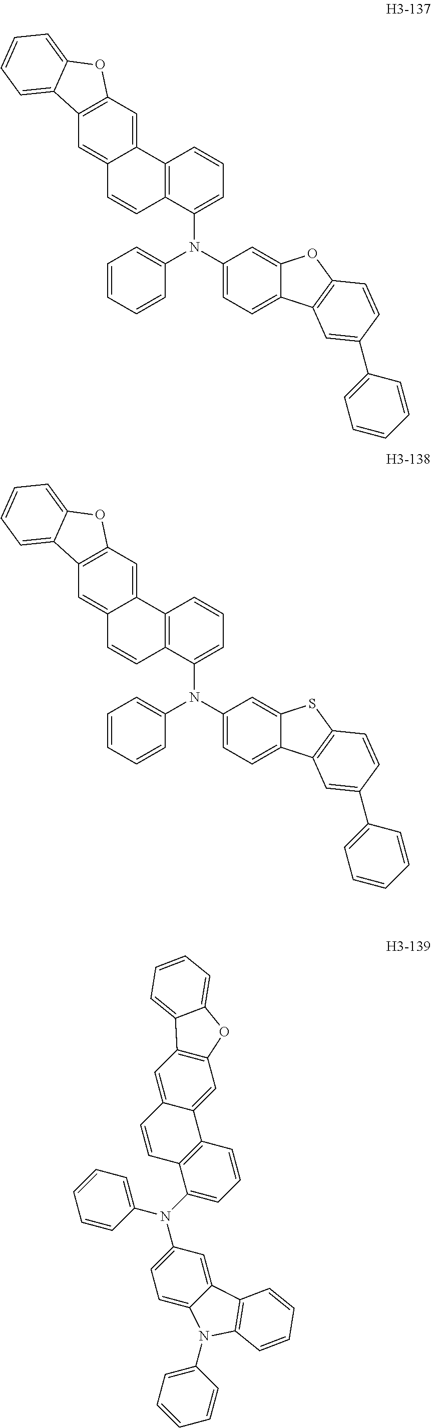 [SMILES: c1ccc(-c2ccc3oc4cc(N(c5ccccc5)c5cccc6c5ccc5cc7c(cc56)oc5ccccc57)ccc4c3c2)cc1 ; c1ccc(-c2ccc3sc4cc(N(c5ccccc5)c5cccc6c5ccc5cc7c(cc56)oc5ccccc57)ccc4c3c2)cc1 ; c1ccc(N(c2ccc3c(c2)c2ccccc2n3-c2ccccc2)c2cccc3c2ccc2cc4c(cc23)oc2ccccc24)cc1]

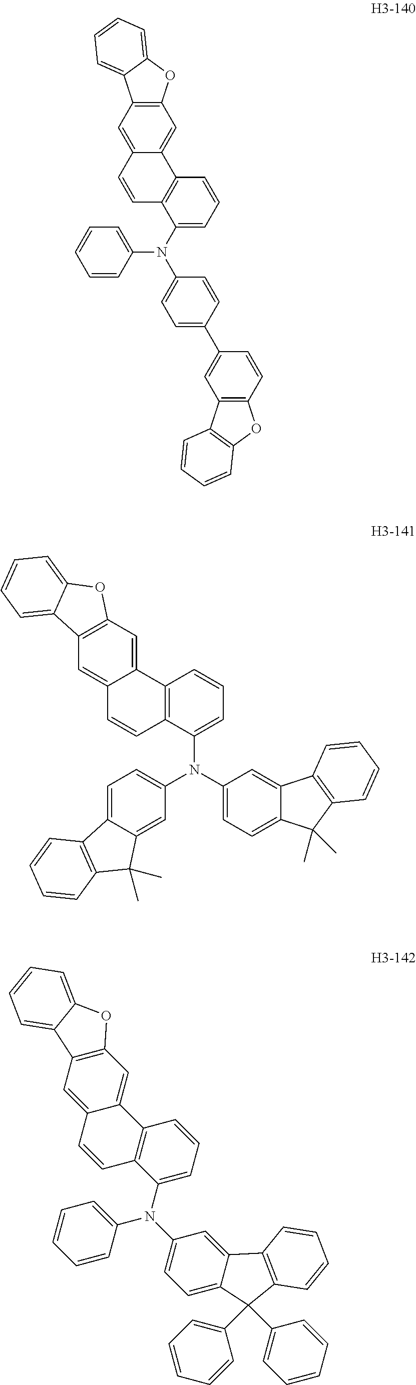 [SMILES: c1ccc(N(c2ccc(-c3ccc4oc5ccccc5c4c3)cc2)c2cccc3c2ccc2cc4c(cc23)oc2ccccc24)cc1 ; CC1(C)c2ccccc2-c2cc(N(c3ccc4c(c3)C(C)(C)c3ccccc3-4)c3cccc4c3ccc3cc5c(cc34)oc3ccccc35)ccc21 ; c1ccc(N(c2ccc3c(c2)-c2ccccc2C3(c2ccccc2)c2ccccc2)c2cccc3c2ccc2cc4c(cc23)oc2ccccc24)cc1]

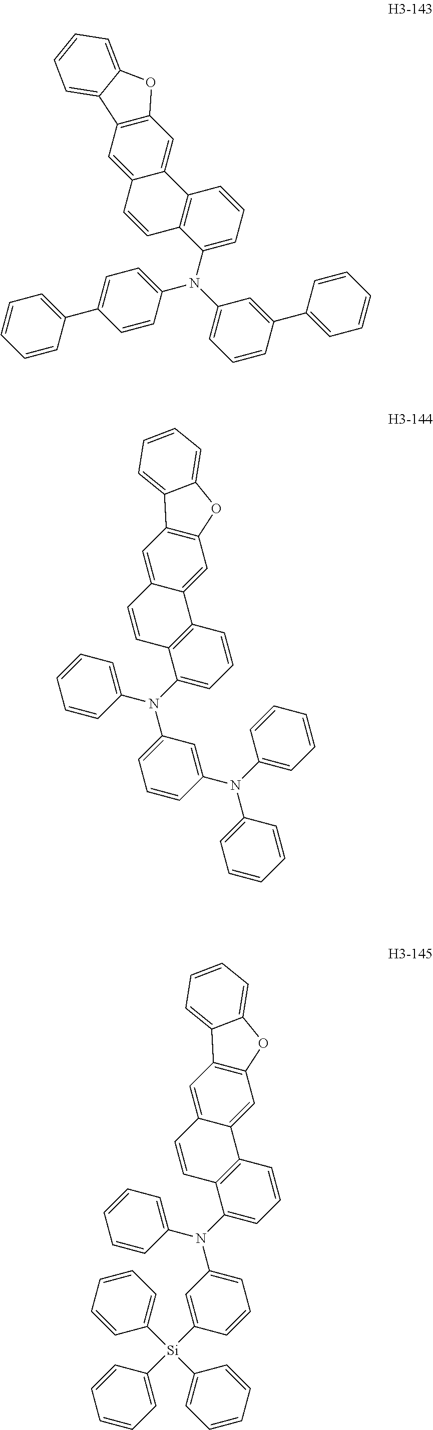 [SMILES: c1ccc(-c2ccc(N(c3cccc(-c4ccccc4)c3)c3cccc4c3ccc3cc5c(cc34)oc3ccccc35)cc2)cc1 ; c1ccc(N(c2ccccc2)c2cccc(N(c3ccccc3)c3cccc4c3ccc3cc5c(cc34)oc3ccccc35)c2)cc1 ; c1ccc(N(c2cccc([Si](c3ccccc3)(c3ccccc3)c3ccccc3)c2)c2cccc3c2ccc2cc4c(cc23)oc2ccccc24)cc1]

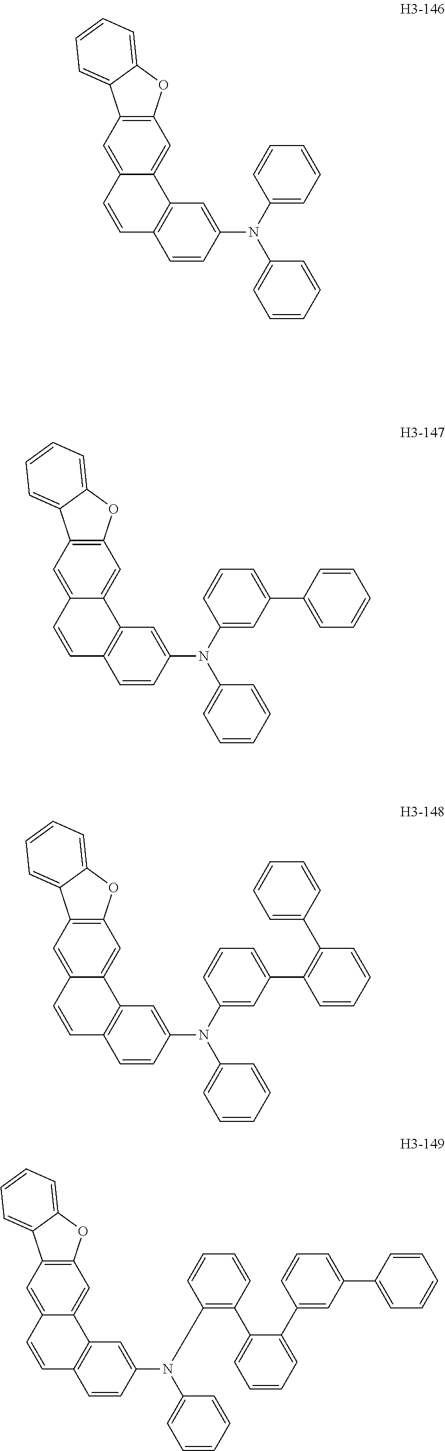 [SMILES: c1ccc(N(c2ccccc2)c2ccc3ccc4cc5c(cc4c3c2)oc2ccccc25)cc1 ; c1ccc(-c2cccc(N(c3ccccc3)c3ccc4ccc5cc6c(cc5c4c3)oc3ccccc36)c2)cc1 ; c1ccc(-c2ccccc2-c2cccc(N(c3ccccc3)c3ccc4ccc5cc6c(cc5c4c3)oc3ccccc36)c2)cc1 ; c1ccc(-c2cccc(-c3ccccc3-c3ccccc3N(c3ccccc3)c3ccc4ccc5cc6c(cc5c4c3)oc3ccccc36)c2)cc1]

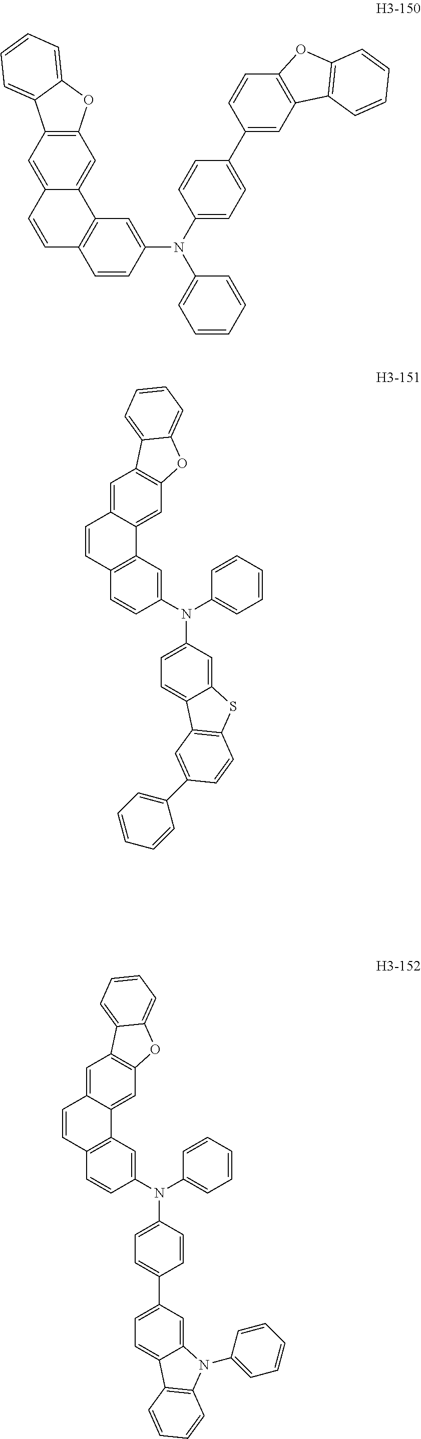 [SMILES: c1ccc(N(c2ccc(-c3ccc4oc5ccccc5c4c3)cc2)c2ccc3ccc4cc5c(cc4c3c2)oc2ccccc25)cc1 ; c1ccc(-c2ccc3sc4cc(N(c5ccccc5)c5ccc6ccc7cc8c(cc7c6c5)oc5ccccc58)ccc4c3c2)cc1 ; c1ccc(N(c2ccc(-c3ccc4c5ccccc5n(-c5ccccc5)c4c3)cc2)c2ccc3ccc4cc5c(cc4c3c2)oc2ccccc25)cc1]

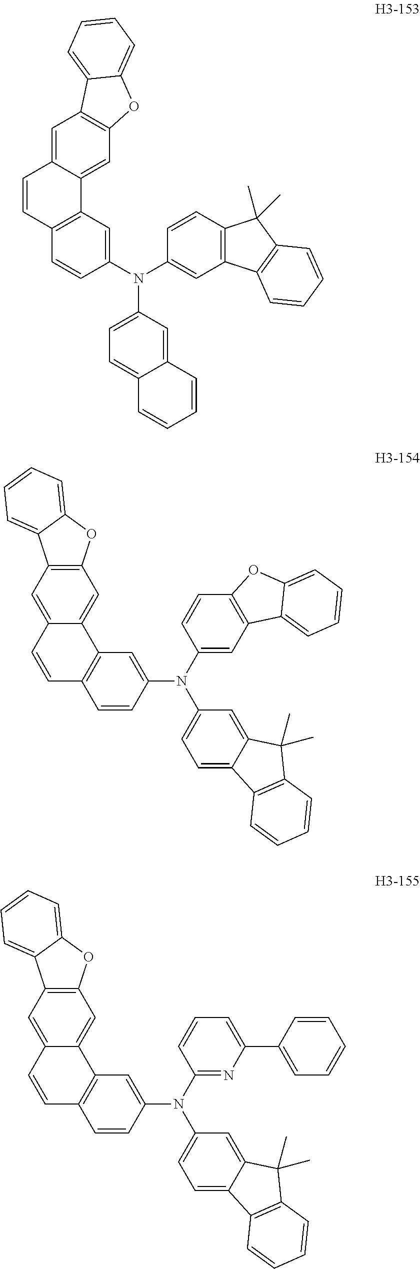 [SMILES: CC1(C)c2ccccc2-c2cc(N(c3ccc4ccccc4c3)c3ccc4ccc5cc6c(cc5c4c3)oc3ccccc36)ccc21 ; CC1(C)c2ccccc2-c2ccc(N(c3ccc4ccc5cc6c(cc5c4c3)oc3ccccc36)c3ccc4oc5ccccc5c4c3)cc21 ; CC1(C)c2ccccc2-c2ccc(N(c3ccc4ccc5cc6c(cc5c4c3)oc3ccccc36)c3cccc(-c4ccccc4)n3)cc21]

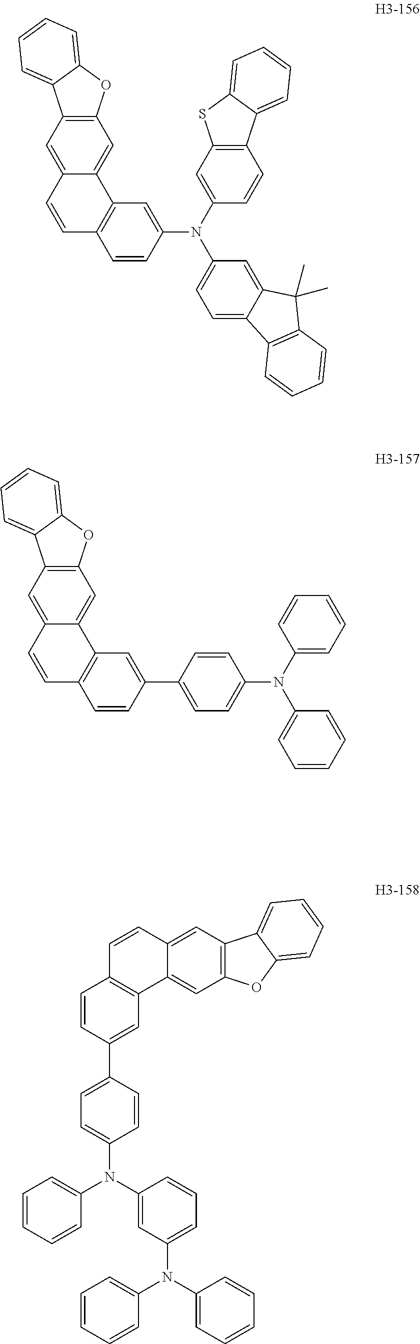 [SMILES: CC1(C)c2ccccc2-c2ccc(N(c3ccc4c(c3)sc3ccccc34)c3ccc4ccc5cc6c(cc5c4c3)oc3ccccc36)cc21 ; c1ccc(N(c2ccccc2)c2ccc(-c3ccc4ccc5cc6c(cc5c4c3)oc3ccccc36)cc2)cc1 ; c1ccc(N(c2ccccc2)c2cccc(N(c3ccccc3)c3ccc(-c4ccc5ccc6cc7c(cc6c5c4)oc4ccccc47)cc3)c2)cc1]

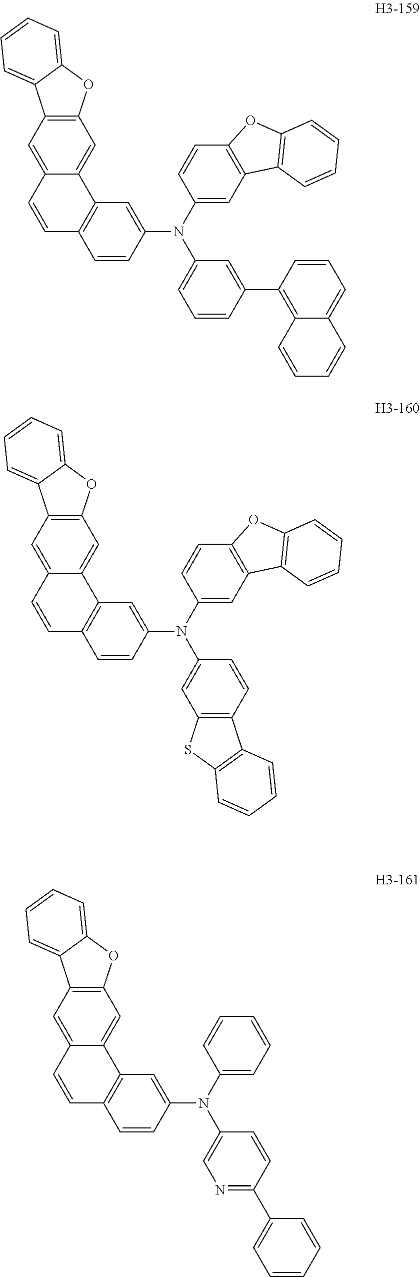 [SMILES: c1cc(-c2cccc3ccccc23)cc(N(c2ccc3ccc4cc5c(cc4c3c2)oc2ccccc25)c2ccc3oc4ccccc4c3c2)c1 ; c1ccc2c(c1)oc1ccc(N(c3ccc4c(c3)sc3ccccc34)c3ccc4ccc5cc6c(cc5c4c3)oc3ccccc36)cc12 ; c1ccc(-c2ccc(N(c3ccccc3)c3ccc4ccc5cc6c(cc5c4c3)oc3ccccc36)cn2)cc1]

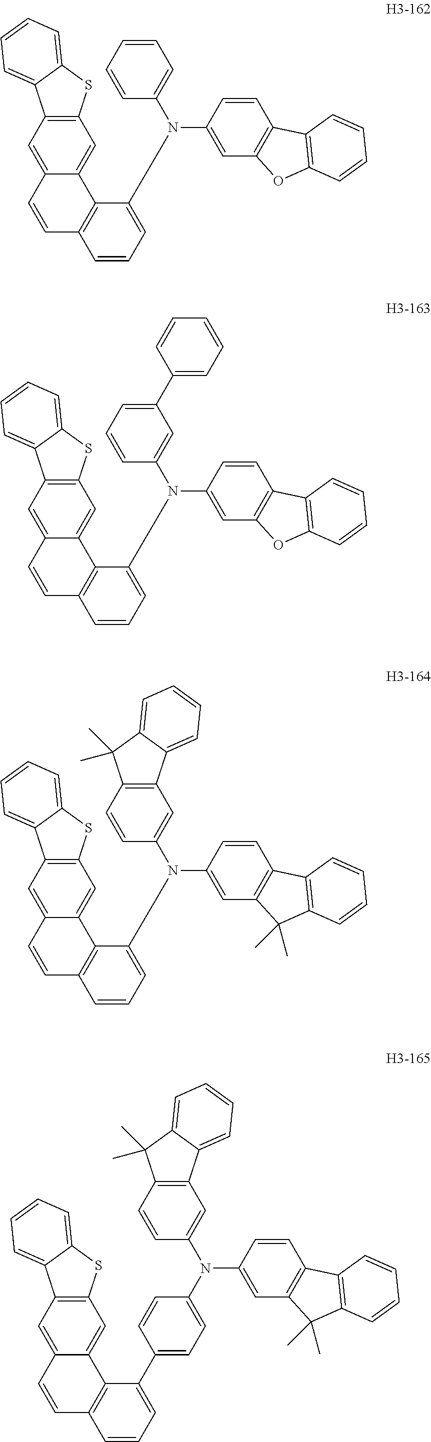 [SMILES: c1ccc(N(c2ccc3c(c2)oc2ccccc23)c2cccc3ccc4cc5c(cc4c23)sc2ccccc25)cc1 ; c1ccc(-c2cccc(N(c3ccc4c(c3)oc3ccccc34)c3cccc4ccc5cc6c(cc5c34)sc3ccccc36)c2)cc1 ; CC1(C)c2ccccc2-c2cc(N(c3ccc4c(c3)C(C)(C)c3ccccc3-4)c3cccc4ccc5cc6c(cc5c34)sc3ccccc36)ccc21 ; CC1(C)c2ccccc2-c2cc(N(c3ccc(-c4cccc5ccc6cc7c(cc6c45)sc4ccccc47)cc3)c3ccc4c(c3)C(C)(C)c3ccccc3-4)ccc21]

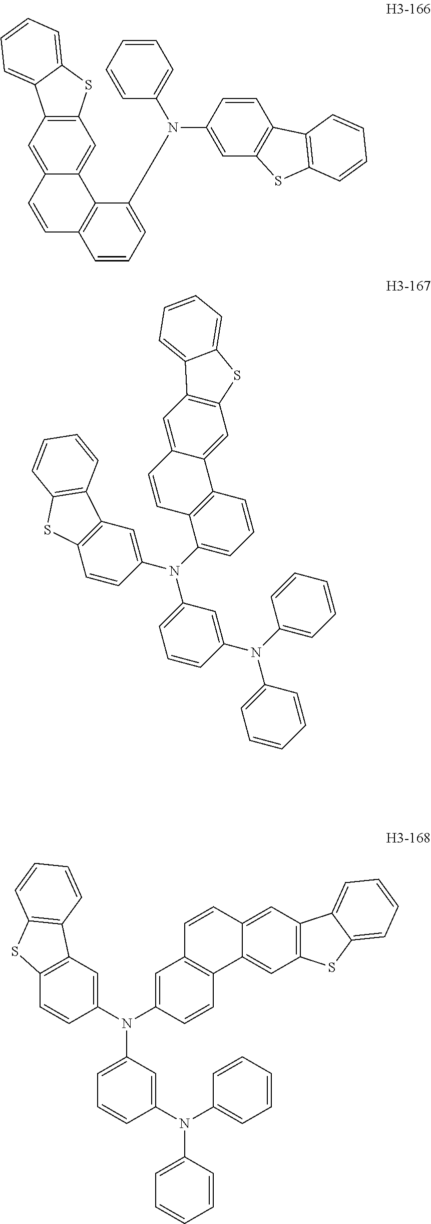 [SMILES: c1ccc(N(c2ccc3c(c2)sc2ccccc23)c2cccc3ccc4cc5c(cc4c23)sc2ccccc25)cc1 ; c1ccc(N(c2ccccc2)c2cccc(N(c3ccc4sc5ccccc5c4c3)c3cccc4c3ccc3cc5c(cc34)sc3ccccc35)c2)cc1 ; c1ccc(N(c2ccccc2)c2cccc(N(c3ccc4c(ccc5cc6c(cc54)sc4ccccc46)c3)c3ccc4sc5ccccc5c4c3)c2)cc1]

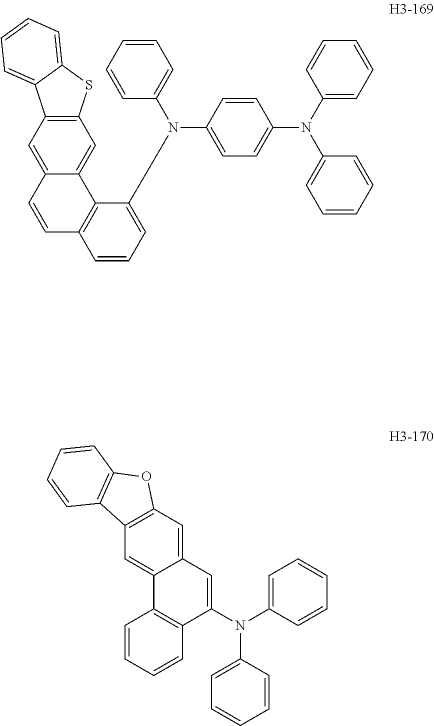 [SMILES: c1ccc(N(c2ccccc2)c2ccc(N(c3ccccc3)c3cccc4ccc5cc6c(cc5c34)sc3ccccc36)cc2)cc1 ; c1ccc(N(c2ccccc2)c2cc3cc4oc5ccccc5c4cc3c3ccccc23)cc1]

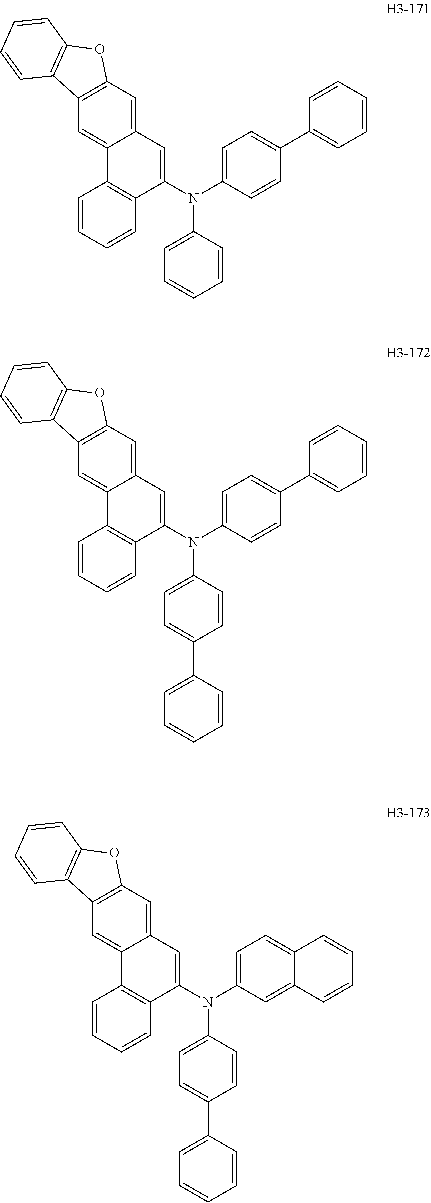 [SMILES: c1ccc(-c2ccc(N(c3ccccc3)c3cc4cc5oc6ccccc6c5cc4c4ccccc34)cc2)cc1 ; c1ccc(-c2ccc(N(c3ccc(-c4ccccc4)cc3)c3cc4cc5oc6ccccc6c5cc4c4ccccc34)cc2)cc1 ; c1ccc(-c2ccc(N(c3ccc4ccccc4c3)c3cc4cc5oc6ccccc6c5cc4c4ccccc34)cc2)cc1]

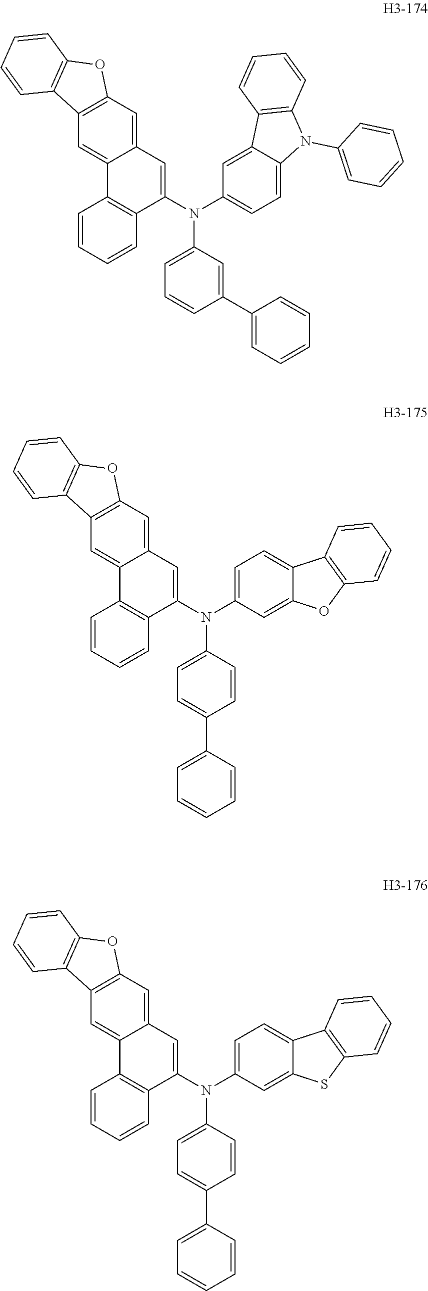 [SMILES: c1ccc(-c2cccc(N(c3ccc4c(c3)c3ccccc3n4-c3ccccc3)c3cc4cc5oc6ccccc6c5cc4c4ccccc34)c2)cc1 ; c1ccc(-c2ccc(N(c3ccc4c(c3)oc3ccccc34)c3cc4cc5oc6ccccc6c5cc4c4ccccc34)cc2)cc1 ; c1ccc(-c2ccc(N(c3ccc4c(c3)sc3ccccc34)c3cc4cc5oc6ccccc6c5cc4c4ccccc34)cc2)cc1]

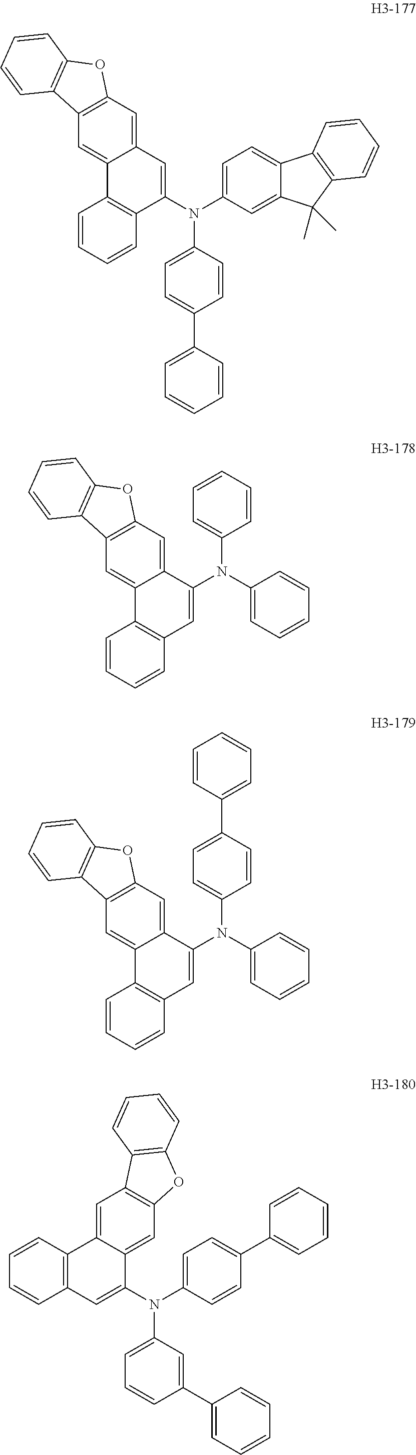 [SMILES: CC1(C)c2ccccc2-c2ccc(N(c3ccc(-c4ccccc4)cc3)c3cc4cc5oc6ccccc6c5cc4c4ccccc34)cc21 ; c1ccc(N(c2ccccc2)c2cc3ccccc3c3cc4c(cc23)oc2ccccc24)cc1 ; c1ccc(-c2ccc(N(c3ccccc3)c3cc4ccccc4c4cc5c(cc34)oc3ccccc35)cc2)cc1 ; c1ccc(-c2ccc(N(c3cccc(-c4ccccc4)c3)c3cc4ccccc4c4cc5c(cc34)oc3ccccc35)cc2)cc1]

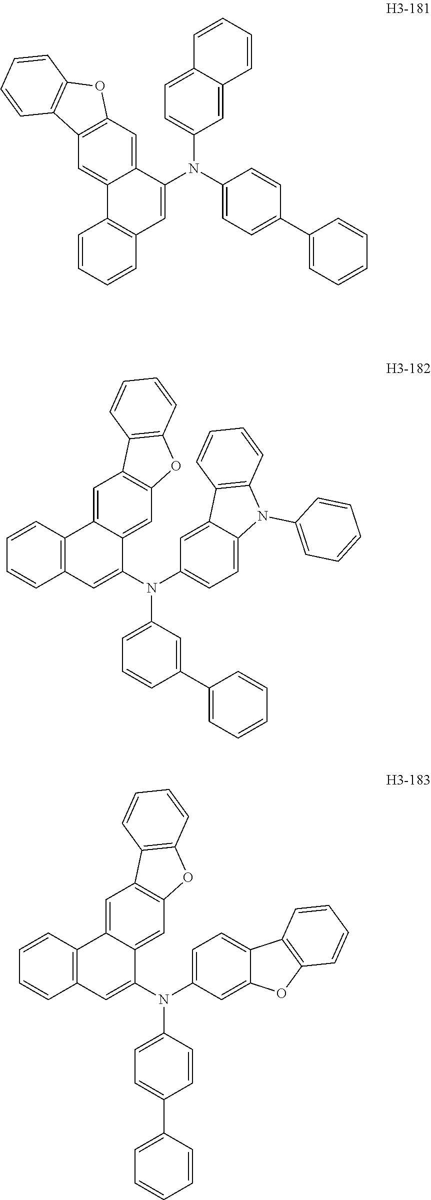 [SMILES: c1ccc(-c2ccc(N(c3ccc4ccccc4c3)c3cc4ccccc4c4cc5c(cc34)oc3ccccc35)cc2)cc1 ; c1ccc(-c2cccc(N(c3ccc4c(c3)c3ccccc3n4-c3ccccc3)c3cc4ccccc4c4cc5c(cc34)oc3ccccc35)c2)cc1 ; c1ccc(-c2ccc(N(c3ccc4c(c3)oc3ccccc34)c3cc4ccccc4c4cc5c(cc34)oc3ccccc35)cc2)cc1]

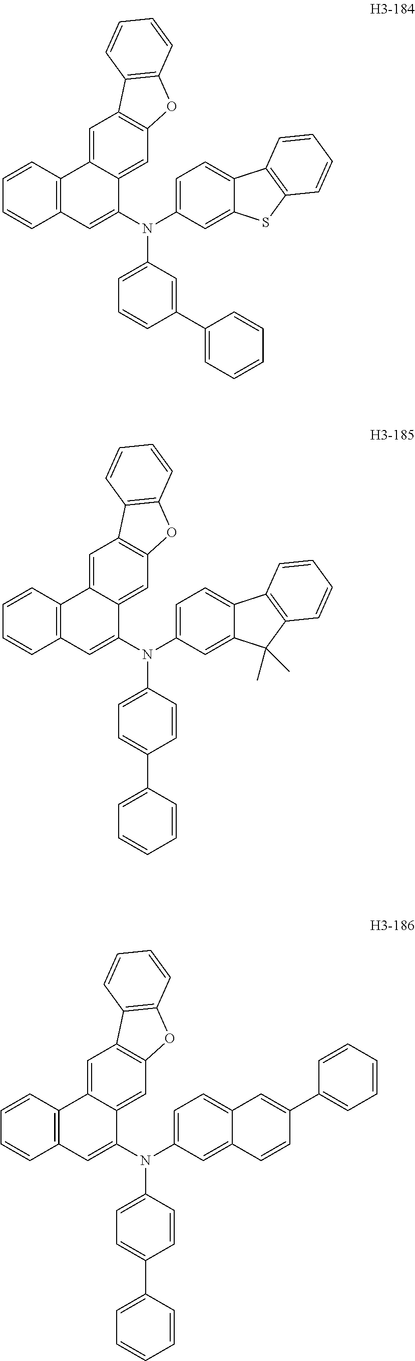 [SMILES: c1ccc(-c2cccc(N(c3ccc4c(c3)sc3ccccc34)c3cc4ccccc4c4cc5c(cc34)oc3ccccc35)c2)cc1 ; CC1(C)c2ccccc2-c2ccc(N(c3ccc(-c4ccccc4)cc3)c3cc4ccccc4c4cc5c(cc34)oc3ccccc35)cc21 ; c1ccc(-c2ccc(N(c3ccc4cc(-c5ccccc5)ccc4c3)c3cc4ccccc4c4cc5c(cc34)oc3ccccc35)cc2)cc1]

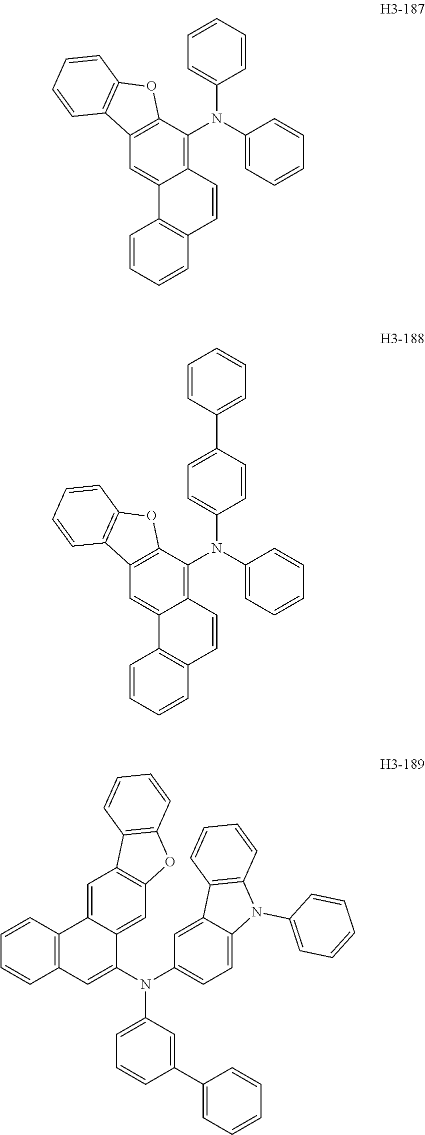 [SMILES: c1ccc(N(c2ccccc2)c2c3ccc4ccccc4c3cc3c2oc2ccccc23)cc1 ; c1ccc(-c2ccc(N(c3ccccc3)c3c4ccc5ccccc5c4cc4c3oc3ccccc34)cc2)cc1 ; c1ccc(-c2cccc(N(c3ccc4c(c3)c3ccccc3n4-c3ccccc3)c3cc4ccccc4c4cc5c(cc34)oc3ccccc35)c2)cc1]

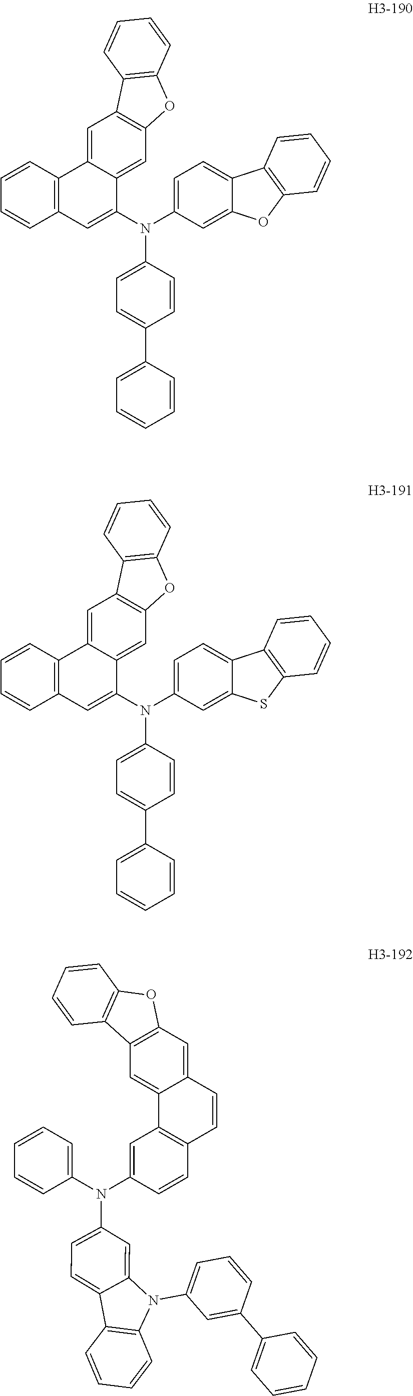 [SMILES: c1ccc(-c2ccc(N(c3ccc4c(c3)oc3ccccc34)c3cc4ccccc4c4cc5c(cc34)oc3ccccc35)cc2)cc1 ; c1ccc(-c2ccc(N(c3ccc4c(c3)sc3ccccc34)c3cc4ccccc4c4cc5c(cc34)oc3ccccc35)cc2)cc1 ; c1ccc(-c2cccc(-n3c4ccccc4c4ccc(N(c5ccccc5)c5ccc6ccc7cc8oc9ccccc9c8cc7c6c5)cc43)c2)cc1]

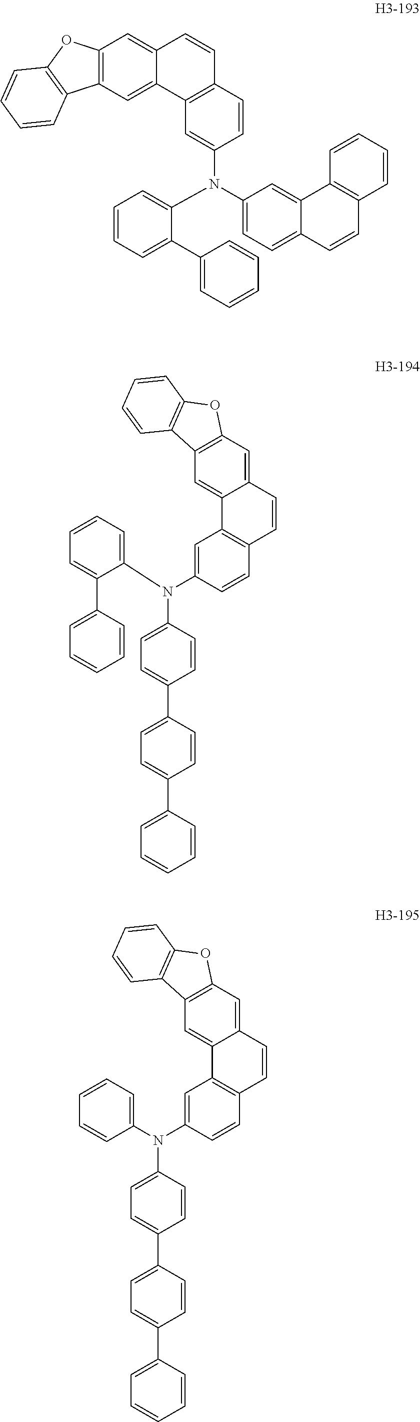 [SMILES: c1ccc(-c2ccccc2N(c2ccc3ccc4ccccc4c3c2)c2ccc3ccc4cc5oc6ccccc6c5cc4c3c2)cc1 ; c1ccc(-c2ccc(-c3ccc(N(c4ccc5ccc6cc7oc8ccccc8c7cc6c5c4)c4ccccc4-c4ccccc4)cc3)cc2)cc1 ; c1ccc(-c2ccc(-c3ccc(N(c4ccccc4)c4ccc5ccc6cc7oc8ccccc8c7cc6c5c4)cc3)cc2)cc1]

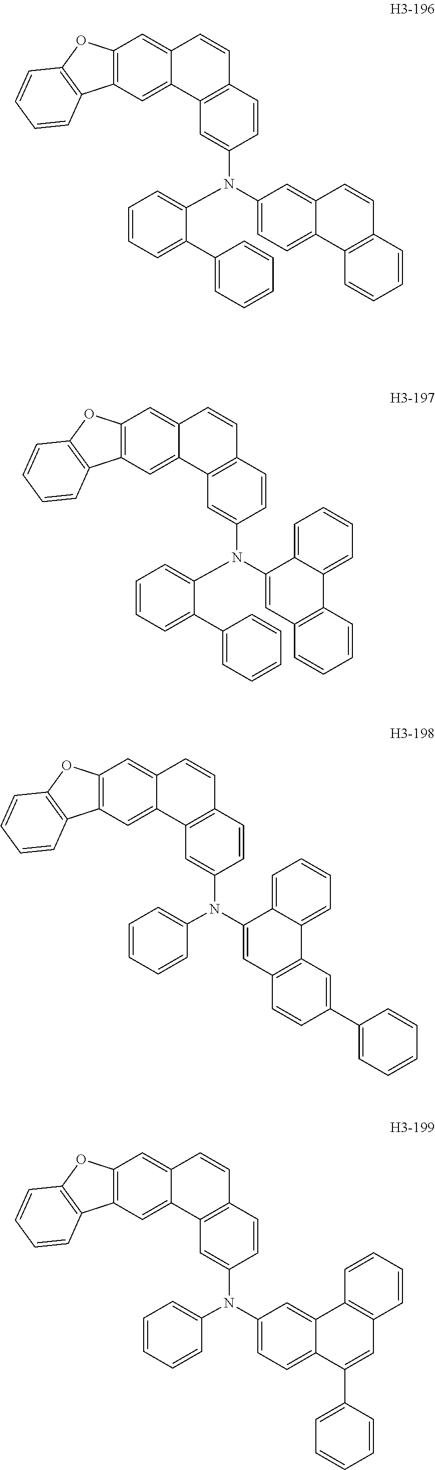 [SMILES: c1ccc(-c2ccccc2N(c2ccc3c(ccc4ccccc43)c2)c2ccc3ccc4cc5oc6ccccc6c5cc4c3c2)cc1 ; c1ccc(-c2ccccc2N(c2ccc3ccc4cc5oc6ccccc6c5cc4c3c2)c2cc3ccccc3c3ccccc23)cc1 ; c1ccc(-c2ccc3cc(N(c4ccccc4)c4ccc5ccc6cc7oc8ccccc8c7cc6c5c4)c4ccccc4c3c2)cc1 ; c1ccc(-c2cc3ccccc3c3cc(N(c4ccccc4)c4ccc5ccc6cc7oc8ccccc8c7cc6c5c4)ccc23)cc1]

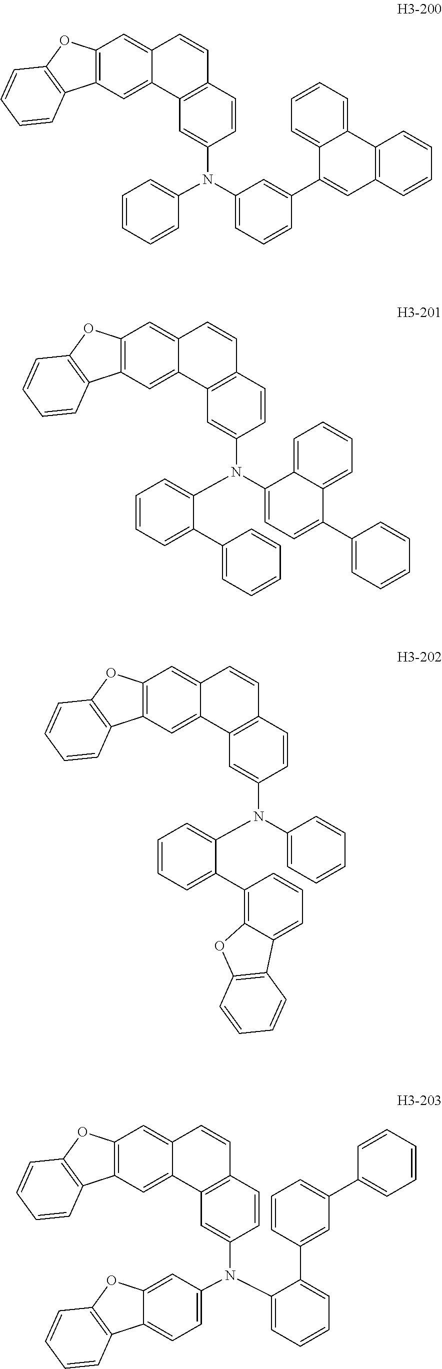 [SMILES: c1ccc(N(c2cccc(-c3cc4ccccc4c4ccccc34)c2)c2ccc3ccc4cc5oc6ccccc6c5cc4c3c2)cc1 ; c1ccc(-c2ccccc2N(c2ccc3ccc4cc5oc6ccccc6c5cc4c3c2)c2ccc(-c3ccccc3)c3ccccc23)cc1 ; c1ccc(N(c2ccc3ccc4cc5oc6ccccc6c5cc4c3c2)c2ccccc2-c2cccc3c2oc2ccccc23)cc1 ; c1ccc(-c2cccc(-c3ccccc3N(c3ccc4c(c3)oc3ccccc34)c3ccc4ccc5cc6oc7ccccc7c6cc5c4c3)c2)cc1]

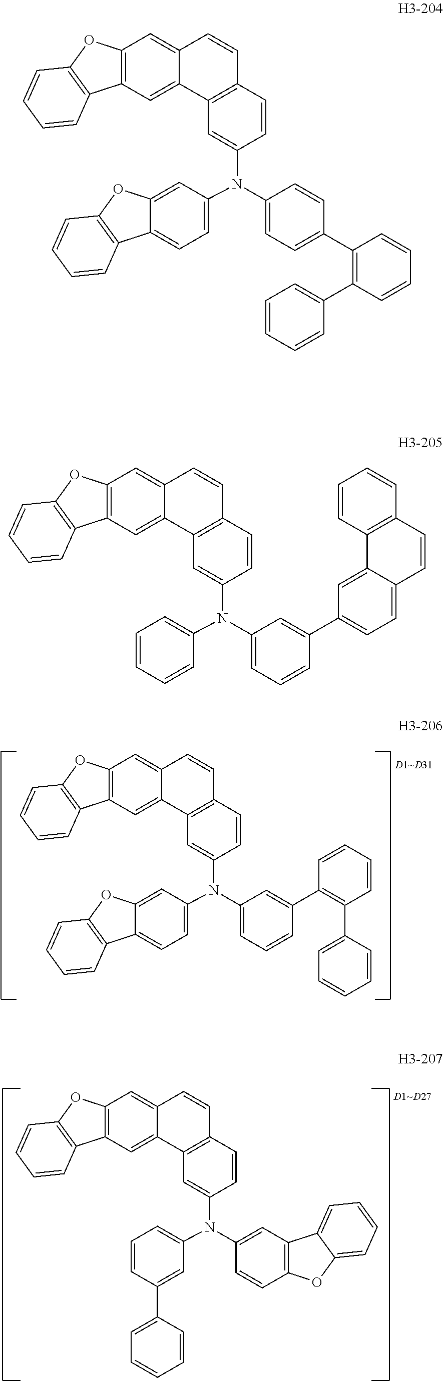

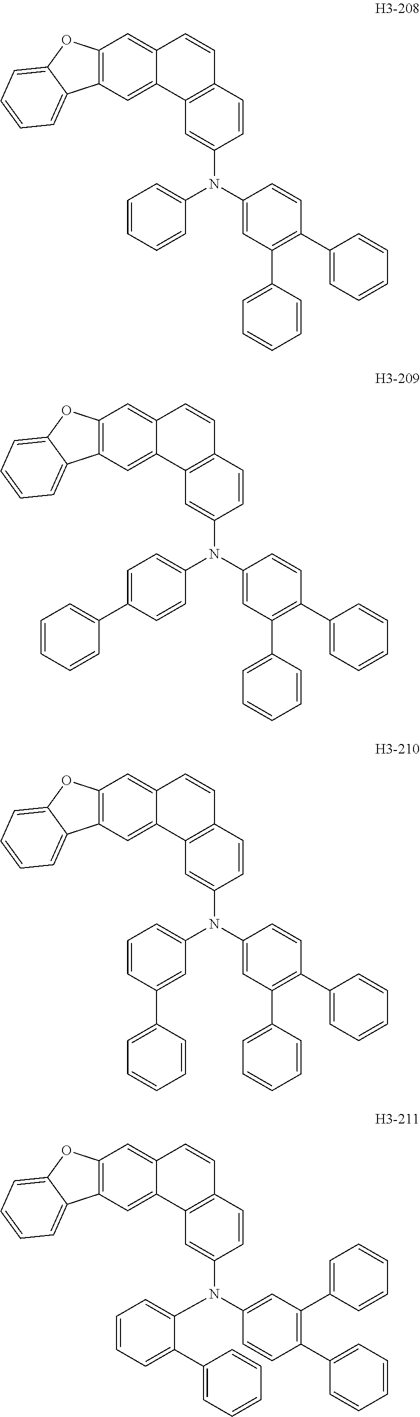 [SMILES: c1ccc(-c2ccc(N(c3ccccc3)c3ccc4ccc5cc6oc7ccccc7c6cc5c4c3)cc2-c2ccccc2)cc1 ; c1ccc(-c2ccc(N(c3ccc(-c4ccccc4)c(-c4ccccc4)c3)c3ccc4ccc5cc6oc7ccccc7c6cc5c4c3)cc2)cc1 ; c1ccc(-c2cccc(N(c3ccc(-c4ccccc4)c(-c4ccccc4)c3)c3ccc4ccc5cc6oc7ccccc7c6cc5c4c3)c2)cc1 ; c1ccc(-c2ccc(N(c3ccc4ccc5cc6oc7ccccc7c6cc5c4c3)c3ccccc3-c3ccccc3)cc2-c2ccccc2)cc1]

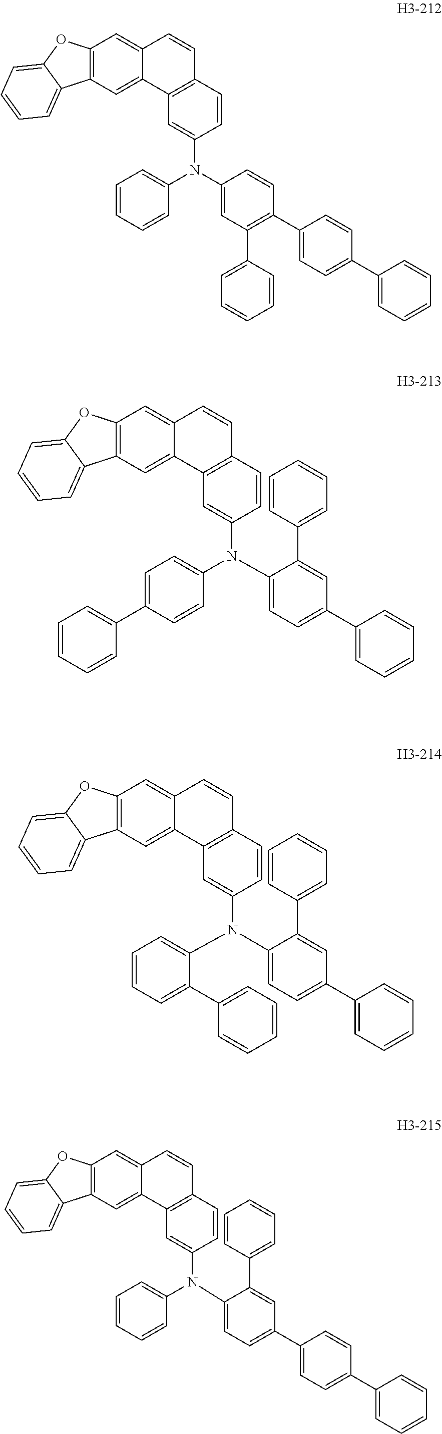 [SMILES: c1ccc(-c2ccc(-c3ccc(N(c4ccccc4)c4ccc5ccc6cc7oc8ccccc8c7cc6c5c4)cc3-c3ccccc3)cc2)cc1 ; c1ccc(-c2ccc(N(c3ccc4ccc5cc6oc7ccccc7c6cc5c4c3)c3ccc(-c4ccccc4)cc3-c3ccccc3)cc2)cc1 ; c1ccc(-c2ccc(N(c3ccc4ccc5cc6oc7ccccc7c6cc5c4c3)c3ccccc3-c3ccccc3)c(-c3ccccc3)c2)cc1 ; c1ccc(-c2ccc(-c3ccc(N(c4ccccc4)c4ccc5ccc6cc7oc8ccccc8c7cc6c5c4)c(-c4ccccc4)c3)cc2)cc1]

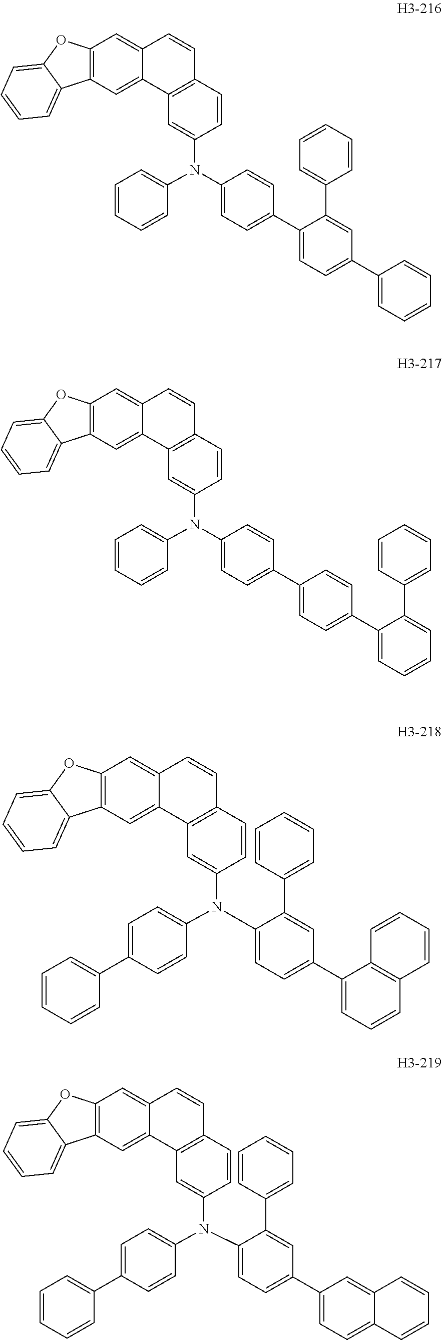 [SMILES: c1ccc(-c2ccc(-c3ccc(N(c4ccccc4)c4ccc5ccc6cc7oc8ccccc8c7cc6c5c4)cc3)c(-c3ccccc3)c2)cc1 ; c1ccc(-c2ccccc2-c2ccc(-c3ccc(N(c4ccccc4)c4ccc5ccc6cc7oc8ccccc8c7cc6c5c4)cc3)cc2)cc1 ; c1ccc(-c2ccc(N(c3ccc4ccc5cc6oc7ccccc7c6cc5c4c3)c3ccc(-c4cccc5ccccc45)cc3-c3ccccc3)cc2)cc1 ; c1ccc(-c2ccc(N(c3ccc4ccc5cc6oc7ccccc7c6cc5c4c3)c3ccc(-c4ccc5ccccc5c4)cc3-c3ccccc3)cc2)cc1]

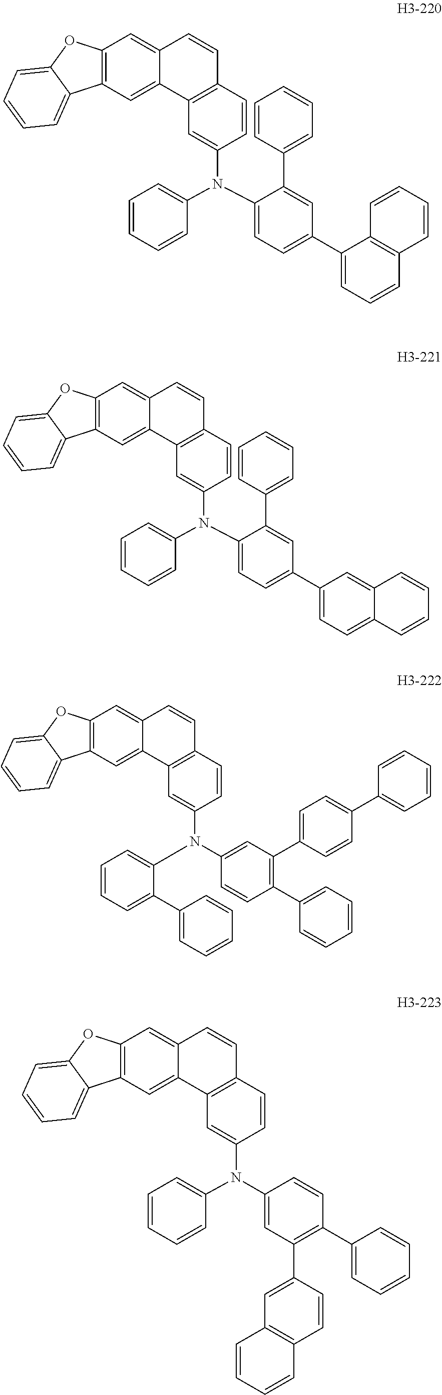 [SMILES: c1ccc(-c2cc(-c3cccc4ccccc34)ccc2N(c2ccccc2)c2ccc3ccc4cc5oc6ccccc6c5cc4c3c2)cc1 ; c1ccc(-c2cc(-c3ccc4ccccc4c3)ccc2N(c2ccccc2)c2ccc3ccc4cc5oc6ccccc6c5cc4c3c2)cc1 ; c1ccc(-c2ccc(-c3cc(N(c4ccc5ccc6cc7oc8ccccc8c7cc6c5c4)c4ccccc4-c4ccccc4)ccc3-c3ccccc3)cc2)cc1 ; c1ccc(-c2ccc(N(c3ccccc3)c3ccc4ccc5cc6oc7ccccc7c6cc5c4c3)cc2-c2ccc3ccccc3c2)cc1]

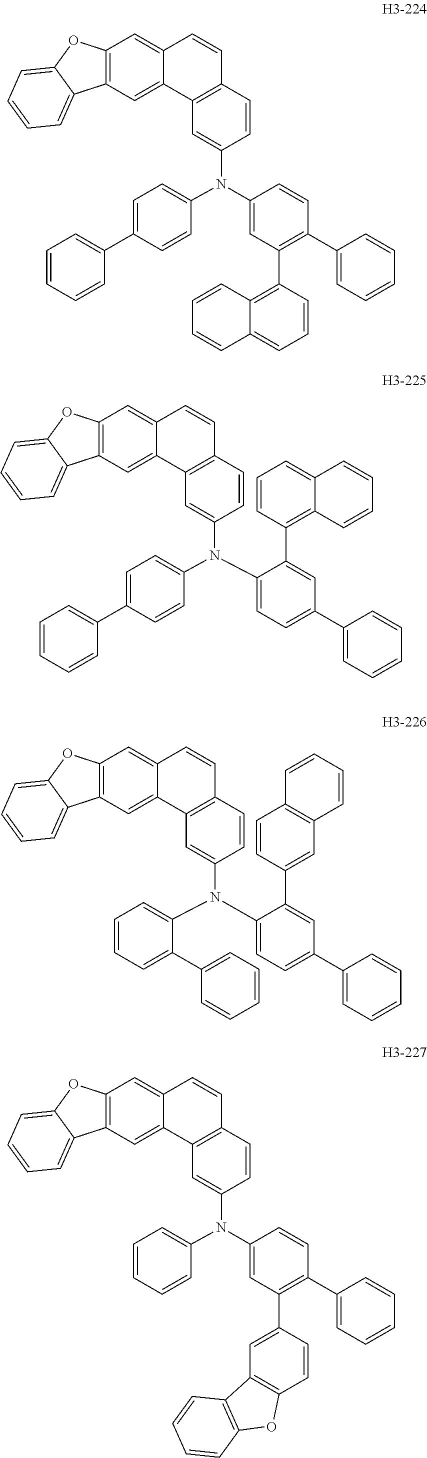 [SMILES: c1ccc(-c2ccc(N(c3ccc(-c4ccccc4)c(-c4cccc5ccccc45)c3)c3ccc4ccc5cc6oc7ccccc7c6cc5c4c3)cc2)cc1 ; c1ccc(-c2ccc(N(c3ccc4ccc5cc6oc7ccccc7c6cc5c4c3)c3ccc(-c4ccccc4)cc3-c3cccc4ccccc34)cc2)cc1 ; c1ccc(-c2ccc(N(c3ccc4ccc5cc6oc7ccccc7c6cc5c4c3)c3ccccc3-c3ccccc3)c(-c3ccc4ccccc4c3)c2)cc1 ; c1ccc(-c2ccc(N(c3ccccc3)c3ccc4ccc5cc6oc7ccccc7c6cc5c4c3)cc2-c2ccc3oc4ccccc4c3c2)cc1]

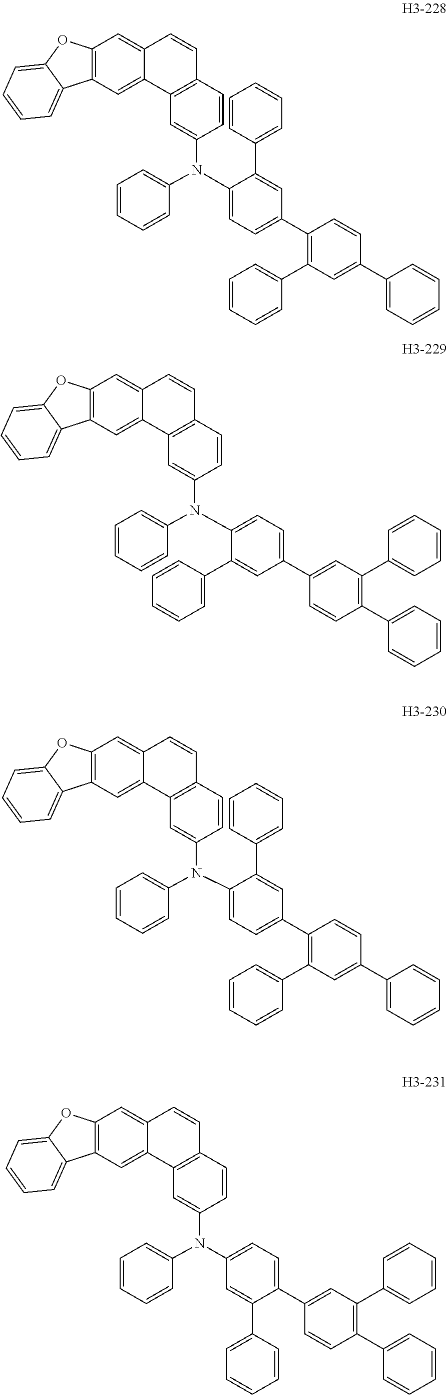 [SMILES: c1ccc(-c2ccc(-c3ccc(N(c4ccccc4)c4ccc5ccc6cc7oc8ccccc8c7cc6c5c4)c(-c4ccccc4)c3)c(-c3ccccc3)c2)cc1 ; c1ccc(-c2ccc(-c3ccc(N(c4ccccc4)c4ccc5ccc6cc7oc8ccccc8c7cc6c5c4)c(-c4ccccc4)c3)cc2-c2ccccc2)cc1 ; c1ccc(-c2ccc(-c3ccc(N(c4ccccc4)c4ccc5ccc6cc7oc8ccccc8c7cc6c5c4)c(-c4ccccc4)c3)c(-c3ccccc3)c2)cc1 ; c1ccc(-c2ccc(-c3ccc(N(c4ccccc4)c4ccc5ccc6cc7oc8ccccc8c7cc6c5c4)cc3-c3ccccc3)cc2-c2ccccc2)cc1]

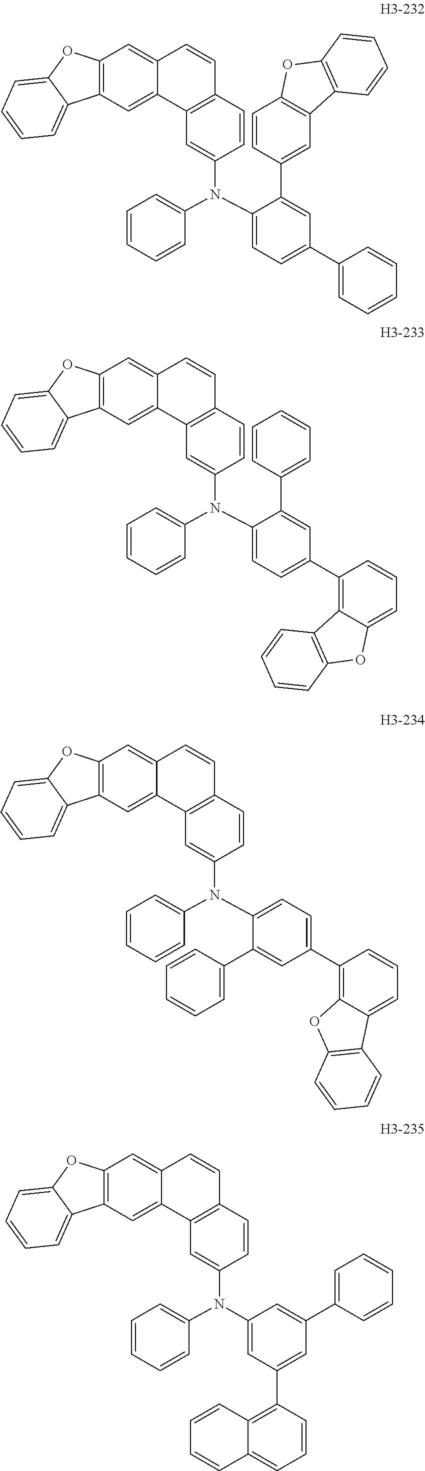 [SMILES: c1ccc(-c2ccc(N(c3ccccc3)c3ccc4ccc5cc6oc7ccccc7c6cc5c4c3)c(-c3ccc4oc5ccccc5c4c3)c2)cc1 ; c1ccc(-c2cc(-c3cccc4oc5ccccc5c34)ccc2N(c2ccccc2)c2ccc3ccc4cc5oc6ccccc6c5cc4c3c2)cc1 ; c1ccc(-c2cc(-c3cccc4c3oc3ccccc34)ccc2N(c2ccccc2)c2ccc3ccc4cc5oc6ccccc6c5cc4c3c2)cc1 ; c1ccc(-c2cc(-c3cccc4ccccc34)cc(N(c3ccccc3)c3ccc4ccc5cc6oc7ccccc7c6cc5c4c3)c2)cc1]

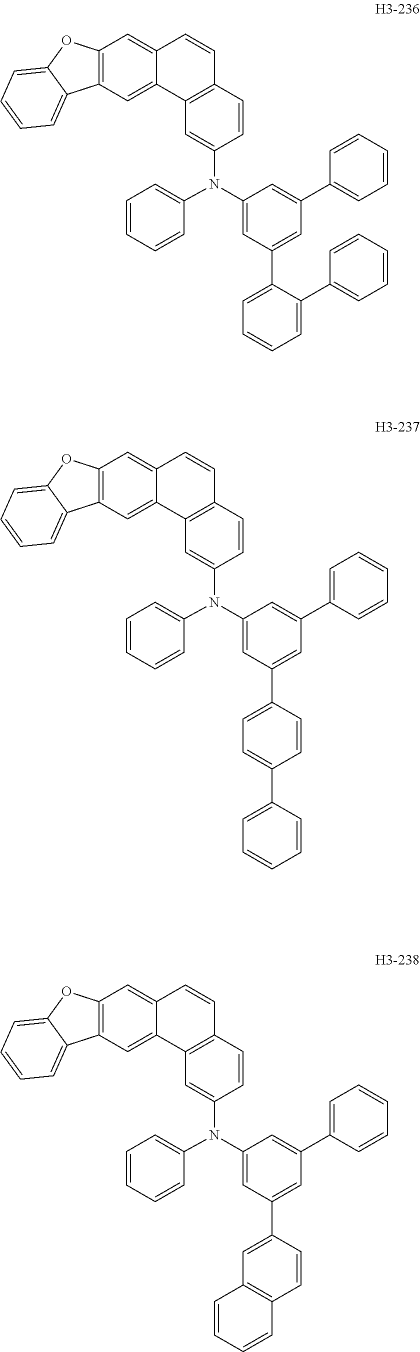 [SMILES: c1ccc(-c2cc(-c3ccccc3-c3ccccc3)cc(N(c3ccccc3)c3ccc4ccc5cc6oc7ccccc7c6cc5c4c3)c2)cc1 ; c1ccc(-c2ccc(-c3cc(-c4ccccc4)cc(N(c4ccccc4)c4ccc5ccc6cc7oc8ccccc8c7cc6c5c4)c3)cc2)cc1 ; c1ccc(-c2cc(-c3ccc4ccccc4c3)cc(N(c3ccccc3)c3ccc4ccc5cc6oc7ccccc7c6cc5c4c3)c2)cc1]

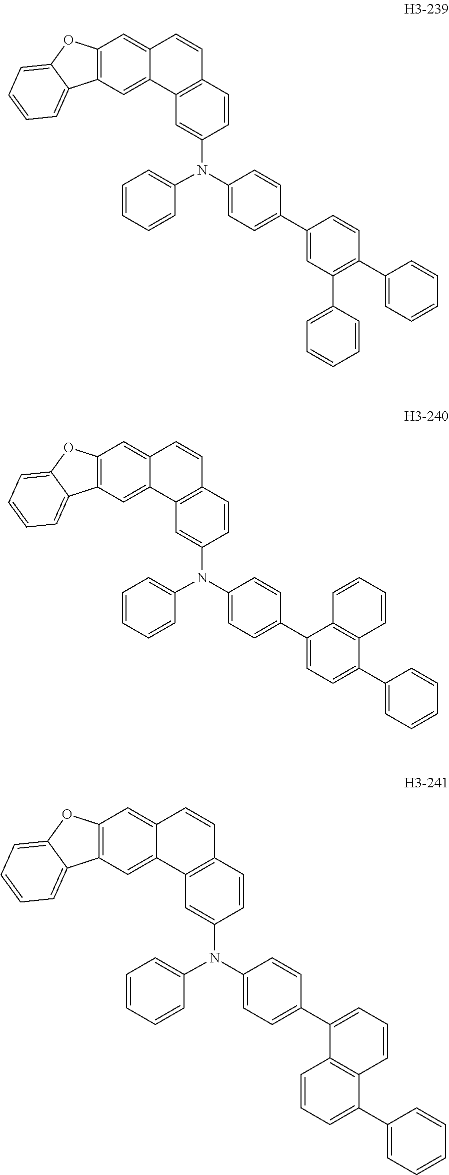 [SMILES: c1ccc(-c2ccc(-c3ccc(N(c4ccccc4)c4ccc5ccc6cc7oc8ccccc8c7cc6c5c4)cc3)cc2-c2ccccc2)cc1 ; c1ccc(-c2ccc(-c3ccc(N(c4ccccc4)c4ccc5ccc6cc7oc8ccccc8c7cc6c5c4)cc3)c3ccccc23)cc1 ; c1ccc(-c2cccc3c(-c4ccc(N(c5ccccc5)c5ccc6ccc7cc8oc9ccccc9c8cc7c6c5)cc4)cccc23)cc1]

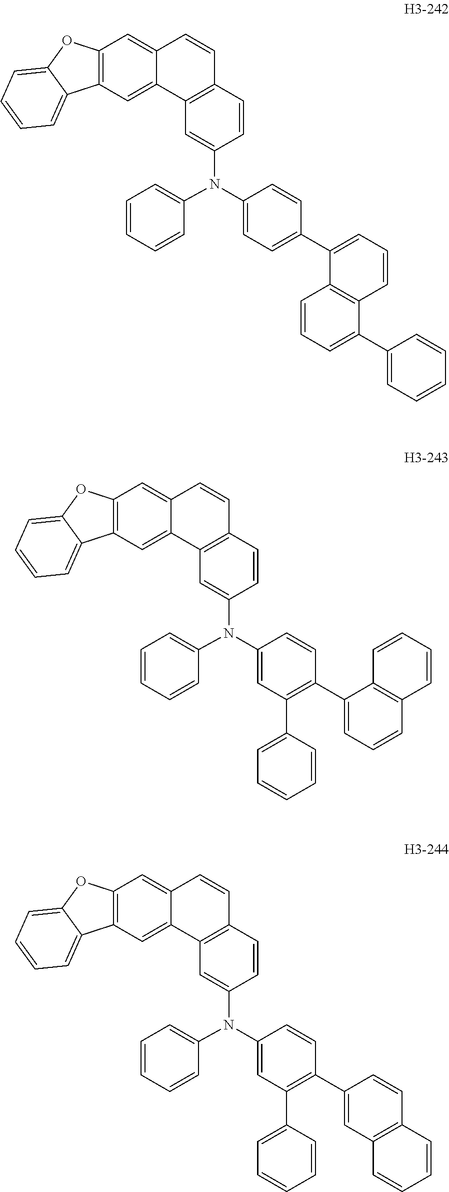 [SMILES: c1ccc(-c2cccc3c(-c4ccc(N(c5ccccc5)c5ccc6ccc7cc8oc9ccccc9c8cc7c6c5)cc4)cccc23)cc1 ; c1ccc(-c2cc(N(c3ccccc3)c3ccc4ccc5cc6oc7ccccc7c6cc5c4c3)ccc2-c2cccc3ccccc23)cc1 ; c1ccc(-c2cc(N(c3ccccc3)c3ccc4ccc5cc6oc7ccccc7c6cc5c4c3)ccc2-c2ccc3ccccc3c2)cc1]

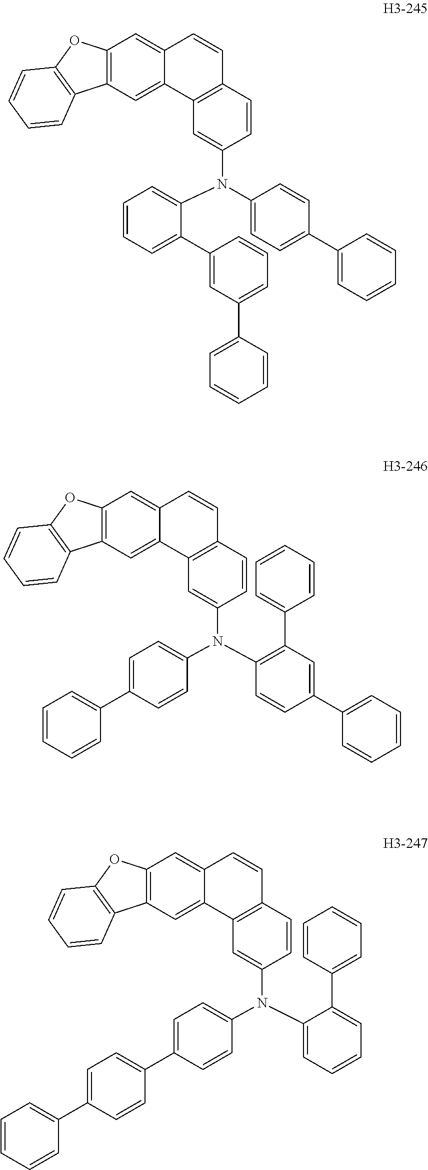 [SMILES: c1ccc(-c2ccc(N(c3ccc4ccc5cc6oc7ccccc7c6cc5c4c3)c3ccccc3-c3cccc(-c4ccccc4)c3)cc2)cc1 ; c1ccc(-c2ccc(N(c3ccc4ccc5cc6oc7ccccc7c6cc5c4c3)c3ccc(-c4ccccc4)cc3-c3ccccc3)cc2)cc1 ; c1ccc(-c2ccc(-c3ccc(N(c4ccc5ccc6cc7oc8ccccc8c7cc6c5c4)c4ccccc4-c4ccccc4)cc3)cc2)cc1]

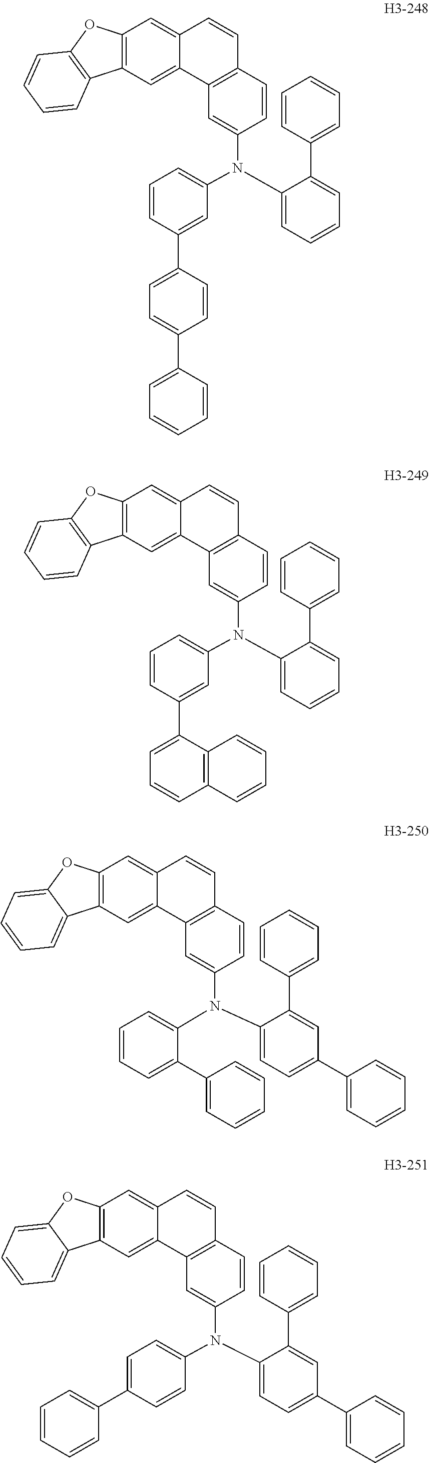 [SMILES: c1ccc(-c2ccc(-c3cccc(N(c4ccc5ccc6cc7oc8ccccc8c7cc6c5c4)c4ccccc4-c4ccccc4)c3)cc2)cc1 ; c1ccc(-c2ccccc2N(c2cccc(-c3cccc4ccccc34)c2)c2ccc3ccc4cc5oc6ccccc6c5cc4c3c2)cc1 ; c1ccc(-c2ccc(N(c3ccc4ccc5cc6oc7ccccc7c6cc5c4c3)c3ccccc3-c3ccccc3)c(-c3ccccc3)c2)cc1 ; c1ccc(-c2ccc(N(c3ccc4ccc5cc6oc7ccccc7c6cc5c4c3)c3ccc(-c4ccccc4)cc3-c3ccccc3)cc2)cc1]

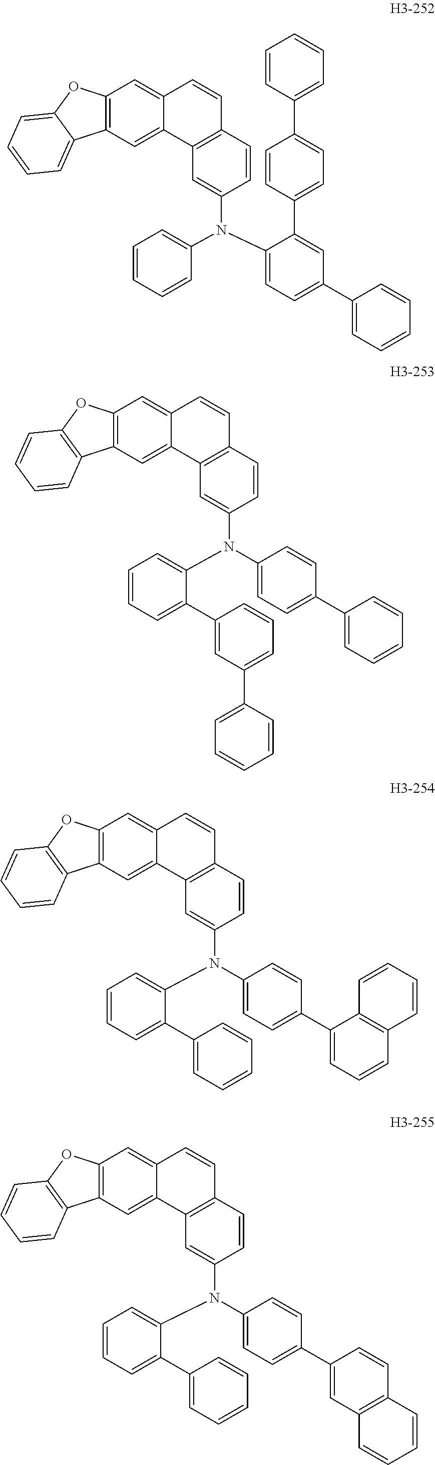 [SMILES: c1ccc(-c2ccc(-c3cc(-c4ccccc4)ccc3N(c3ccccc3)c3ccc4ccc5cc6oc7ccccc7c6cc5c4c3)cc2)cc1 ; c1ccc(-c2ccc(N(c3ccc4ccc5cc6oc7ccccc7c6cc5c4c3)c3ccccc3-c3cccc(-c4ccccc4)c3)cc2)cc1 ; c1ccc(-c2ccccc2N(c2ccc(-c3cccc4ccccc34)cc2)c2ccc3ccc4cc5oc6ccccc6c5cc4c3c2)cc1 ; c1ccc(-c2ccccc2N(c2ccc(-c3ccc4ccccc4c3)cc2)c2ccc3ccc4cc5oc6ccccc6c5cc4c3c2)cc1]

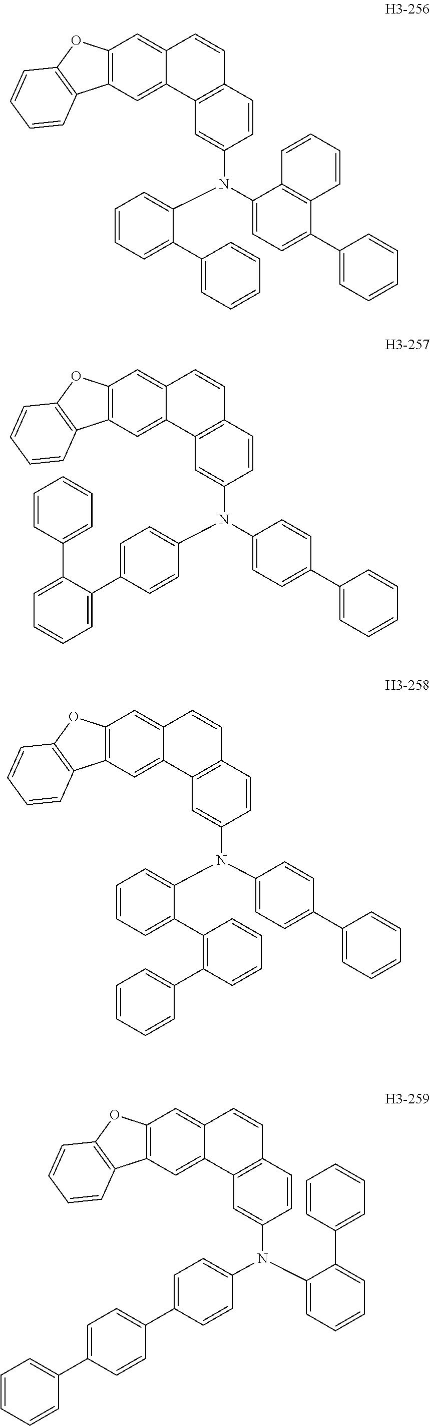 [SMILES: c1ccc(-c2ccccc2N(c2ccc3ccc4cc5oc6ccccc6c5cc4c3c2)c2ccc(-c3ccccc3)c3ccccc23)cc1 ; c1ccc(-c2ccc(N(c3ccc(-c4ccccc4-c4ccccc4)cc3)c3ccc4ccc5cc6oc7ccccc7c6cc5c4c3)cc2)cc1 ; c1ccc(-c2ccc(N(c3ccc4ccc5cc6oc7ccccc7c6cc5c4c3)c3ccccc3-c3ccccc3-c3ccccc3)cc2)cc1 ; c1ccc(-c2ccc(-c3ccc(N(c4ccc5ccc6cc7oc8ccccc8c7cc6c5c4)c4ccccc4-c4ccccc4)cc3)cc2)cc1]

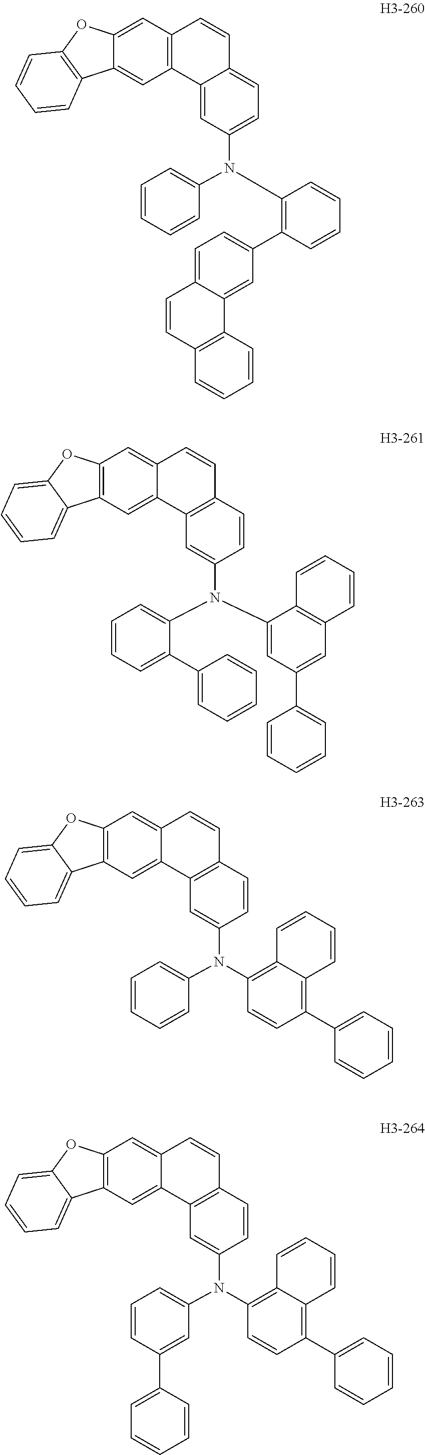 [SMILES: c1ccc(N(c2ccc3ccc4cc5oc6ccccc6c5cc4c3c2)c2ccccc2-c2ccc3ccc4ccccc4c3c2)cc1 ; c1ccc(-c2cc(N(c3ccc4ccc5cc6oc7ccccc7c6cc5c4c3)c3ccccc3-c3ccccc3)c3ccccc3c2)cc1 ; c1ccc(-c2ccc(N(c3ccccc3)c3ccc4ccc5cc6oc7ccccc7c6cc5c4c3)c3ccccc23)cc1 ; c1ccc(-c2cccc(N(c3ccc4ccc5cc6oc7ccccc7c6cc5c4c3)c3ccc(-c4ccccc4)c4ccccc34)c2)cc1]

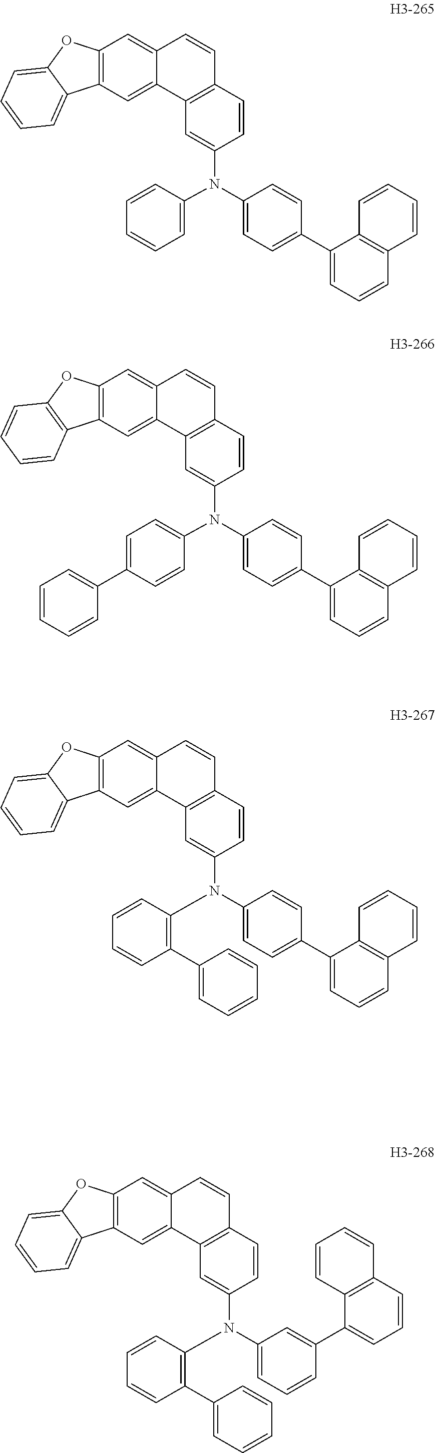 [SMILES: c1ccc(N(c2ccc(-c3cccc4ccccc34)cc2)c2ccc3ccc4cc5oc6ccccc6c5cc4c3c2)cc1 ; c1ccc(-c2ccc(N(c3ccc(-c4cccc5ccccc45)cc3)c3ccc4ccc5cc6oc7ccccc7c6cc5c4c3)cc2)cc1 ; c1ccc(-c2ccccc2N(c2ccc(-c3cccc4ccccc34)cc2)c2ccc3ccc4cc5oc6ccccc6c5cc4c3c2)cc1 ; c1ccc(-c2ccccc2N(c2cccc(-c3cccc4ccccc34)c2)c2ccc3ccc4cc5oc6ccccc6c5cc4c3c2)cc1]

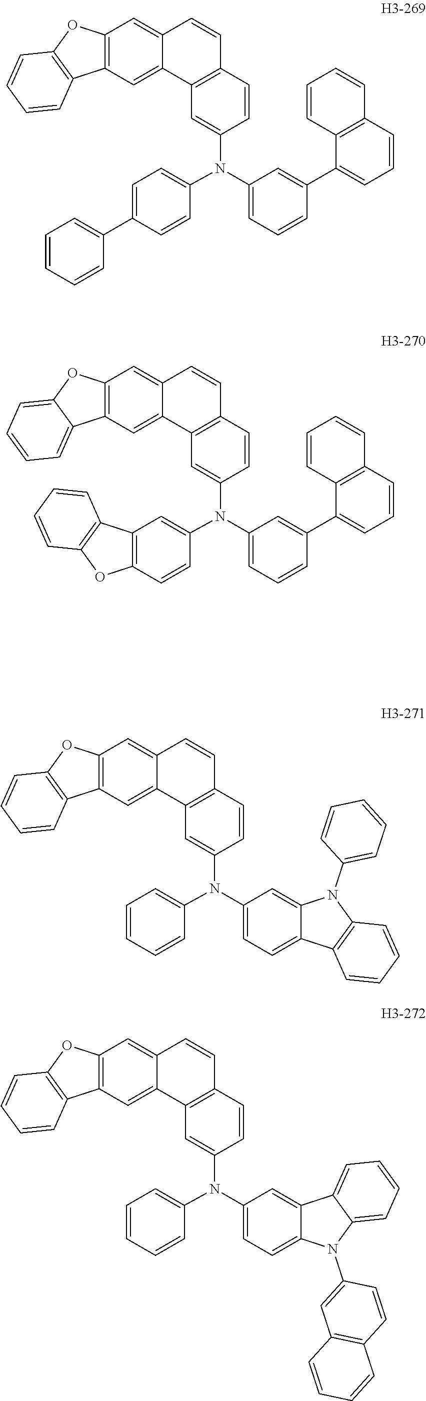 [SMILES: c1ccc(-c2ccc(N(c3cccc(-c4cccc5ccccc45)c3)c3ccc4ccc5cc6oc7ccccc7c6cc5c4c3)cc2)cc1 ; c1cc(-c2cccc3ccccc23)cc(N(c2ccc3ccc4cc5oc6ccccc6c5cc4c3c2)c2ccc3oc4ccccc4c3c2)c1 ; c1ccc(N(c2ccc3ccc4cc5oc6ccccc6c5cc4c3c2)c2ccc3c4ccccc4n(-c4ccccc4)c3c2)cc1 ; c1ccc(N(c2ccc3ccc4cc5oc6ccccc6c5cc4c3c2)c2ccc3c(c2)c2ccccc2n3-c2ccc3ccccc3c2)cc1]

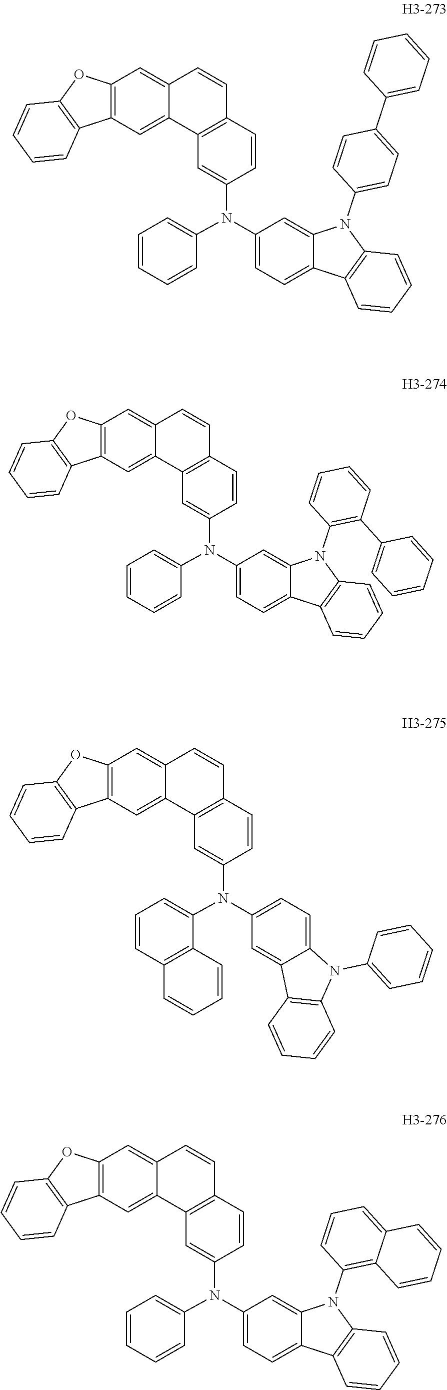 [SMILES: c1ccc(-c2ccc(-n3c4ccccc4c4ccc(N(c5ccccc5)c5ccc6ccc7cc8oc9ccccc9c8cc7c6c5)cc43)cc2)cc1 ; c1ccc(-c2ccccc2-n2c3ccccc3c3ccc(N(c4ccccc4)c4ccc5ccc6cc7oc8ccccc8c7cc6c5c4)cc32)cc1 ; c1ccc(-n2c3ccccc3c3cc(N(c4ccc5ccc6cc7oc8ccccc8c7cc6c5c4)c4cccc5ccccc45)ccc32)cc1 ; c1ccc(N(c2ccc3ccc4cc5oc6ccccc6c5cc4c3c2)c2ccc3c4ccccc4n(-c4cccc5ccccc45)c3c2)cc1]

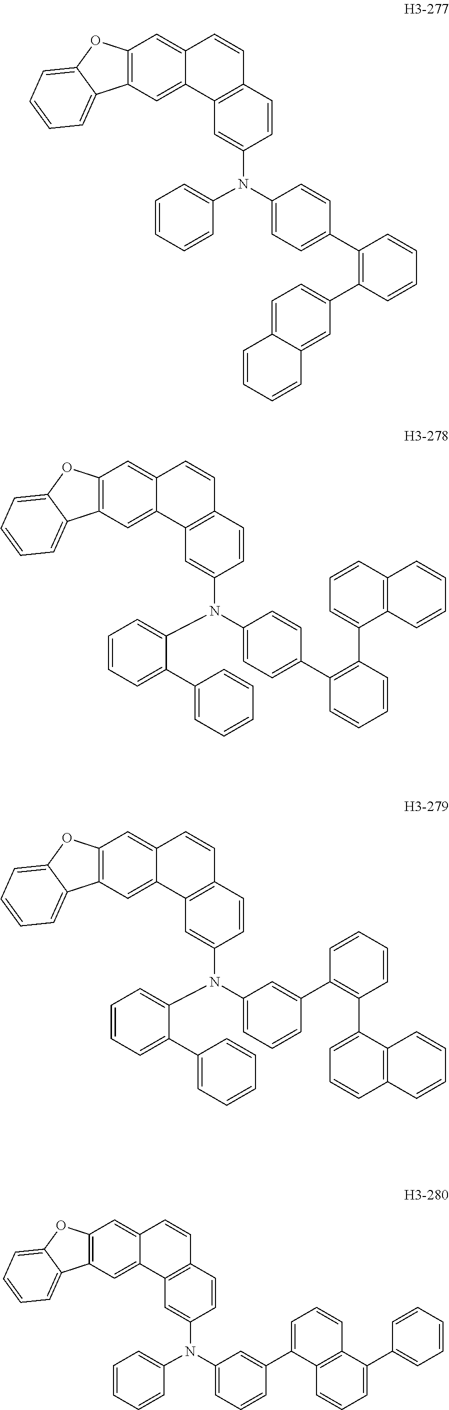 [SMILES: c1ccc(N(c2ccc(-c3ccccc3-c3ccc4ccccc4c3)cc2)c2ccc3ccc4cc5oc6ccccc6c5cc4c3c2)cc1 ; c1ccc(-c2ccccc2N(c2ccc(-c3ccccc3-c3cccc4ccccc34)cc2)c2ccc3ccc4cc5oc6ccccc6c5cc4c3c2)cc1 ; c1ccc(-c2ccccc2N(c2cccc(-c3ccccc3-c3cccc4ccccc34)c2)c2ccc3ccc4cc5oc6ccccc6c5cc4c3c2)cc1 ; c1ccc(-c2cccc3c(-c4cccc(N(c5ccccc5)c5ccc6ccc7cc8oc9ccccc9c8cc7c6c5)c4)cccc23)cc1]

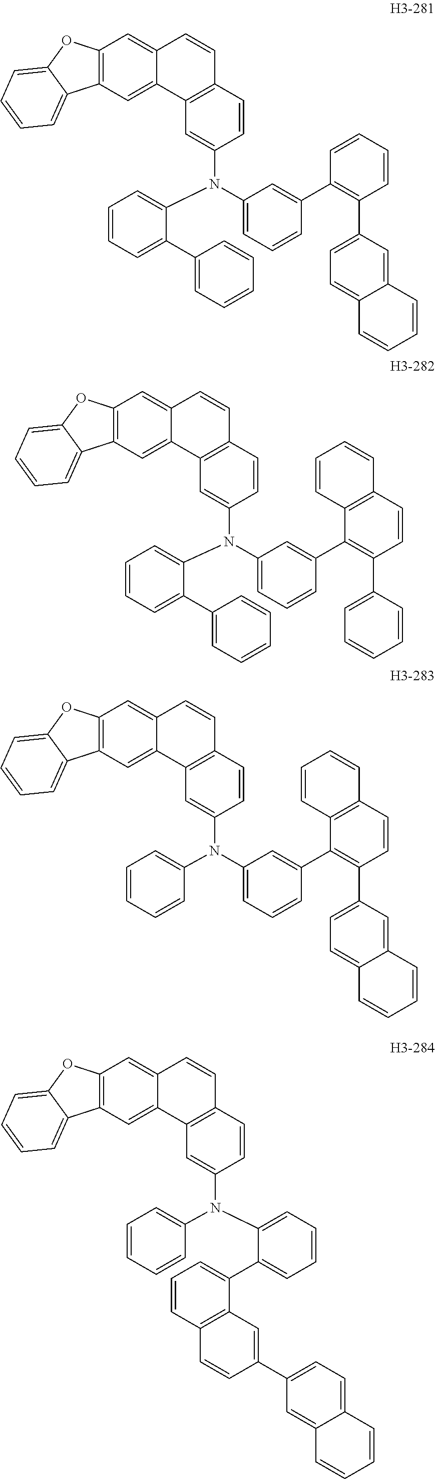 [SMILES: c1ccc(-c2ccccc2N(c2cccc(-c3ccccc3-c3ccc4ccccc4c3)c2)c2ccc3ccc4cc5oc6ccccc6c5cc4c3c2)cc1 ; c1ccc(-c2ccccc2N(c2cccc(-c3c(-c4ccccc4)ccc4ccccc34)c2)c2ccc3ccc4cc5oc6ccccc6c5cc4c3c2)cc1 ; c1ccc(N(c2cccc(-c3c(-c4ccc5ccccc5c4)ccc4ccccc34)c2)c2ccc3ccc4cc5oc6ccccc6c5cc4c3c2)cc1 ; c1ccc(N(c2ccc3ccc4cc5oc6ccccc6c5cc4c3c2)c2ccccc2-c2cccc3ccc(-c4ccc5ccccc5c4)cc23)cc1]

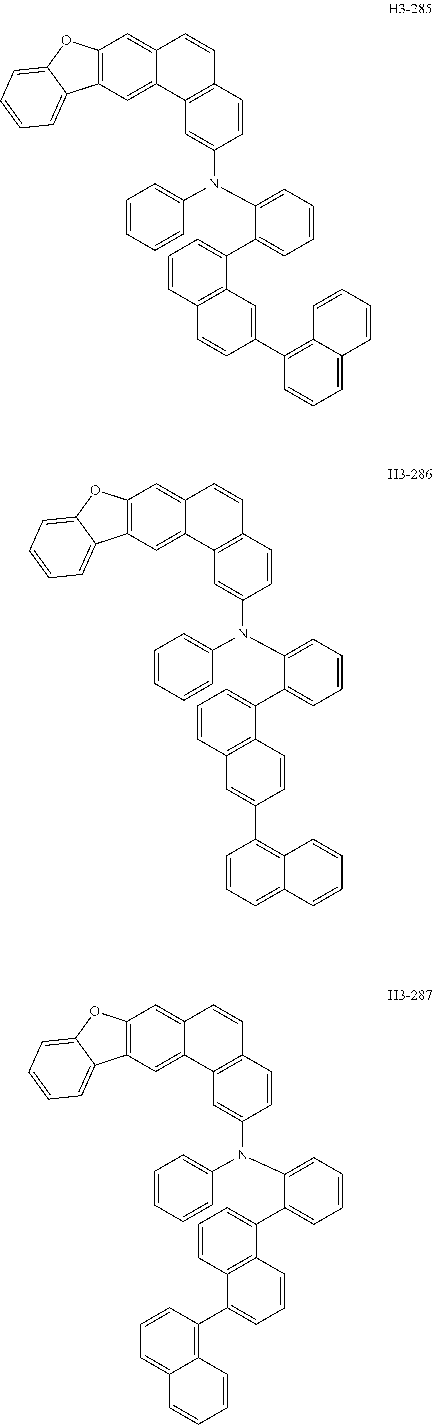 [SMILES: c1ccc(N(c2ccc3ccc4cc5oc6ccccc6c5cc4c3c2)c2ccccc2-c2cccc3ccc(-c4cccc5ccccc45)cc23)cc1 ; c1ccc(N(c2ccc3ccc4cc5oc6ccccc6c5cc4c3c2)c2ccccc2-c2cccc3cc(-c4cccc5ccccc45)ccc23)cc1 ; c1ccc(N(c2ccc3ccc4cc5oc6ccccc6c5cc4c3c2)c2ccccc2-c2cccc3c(-c4cccc5ccccc45)cccc23)cc1]

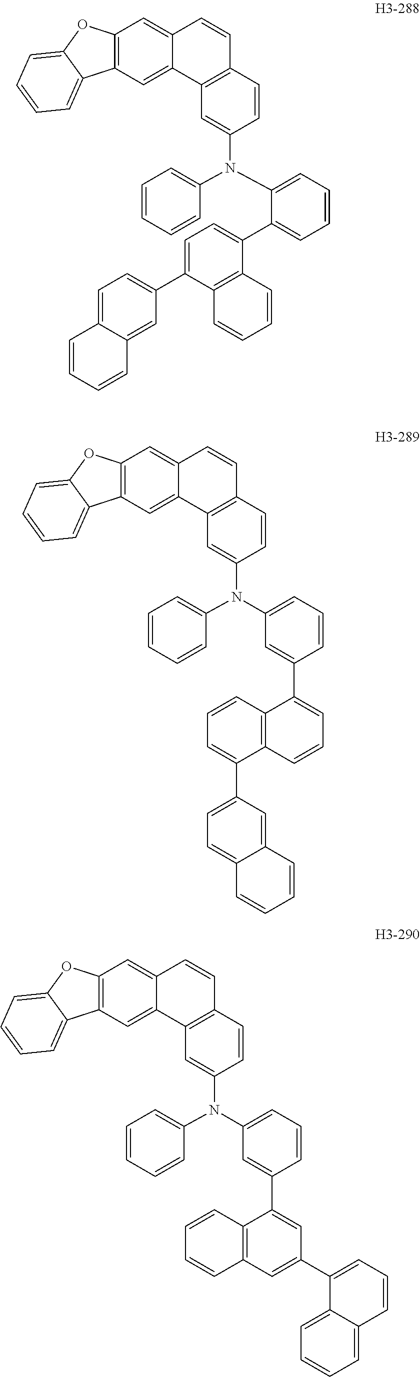 [SMILES: c1ccc(N(c2ccc3ccc4cc5oc6ccccc6c5cc4c3c2)c2ccccc2-c2ccc(-c3ccc4ccccc4c3)c3ccccc23)cc1 ; c1ccc(N(c2cccc(-c3cccc4c(-c5ccc6ccccc6c5)cccc34)c2)c2ccc3ccc4cc5oc6ccccc6c5cc4c3c2)cc1 ; c1ccc(N(c2cccc(-c3cc(-c4cccc5ccccc45)cc4ccccc34)c2)c2ccc3ccc4cc5oc6ccccc6c5cc4c3c2)cc1]

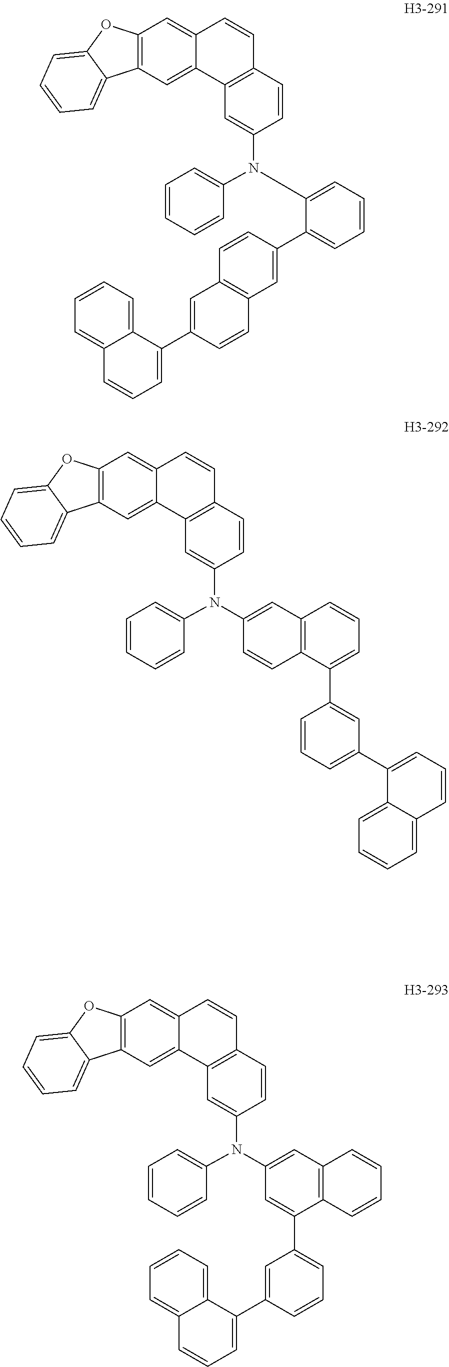 [SMILES: c1ccc(N(c2ccc3ccc4cc5oc6ccccc6c5cc4c3c2)c2ccccc2-c2ccc3cc(-c4cccc5ccccc45)ccc3c2)cc1 ; c1ccc(N(c2ccc3c(-c4cccc(-c5cccc6ccccc56)c4)cccc3c2)c2ccc3ccc4cc5oc6ccccc6c5cc4c3c2)cc1 ; c1ccc(N(c2cc(-c3cccc(-c4cccc5ccccc45)c3)c3ccccc3c2)c2ccc3ccc4cc5oc6ccccc6c5cc4c3c2)cc1]

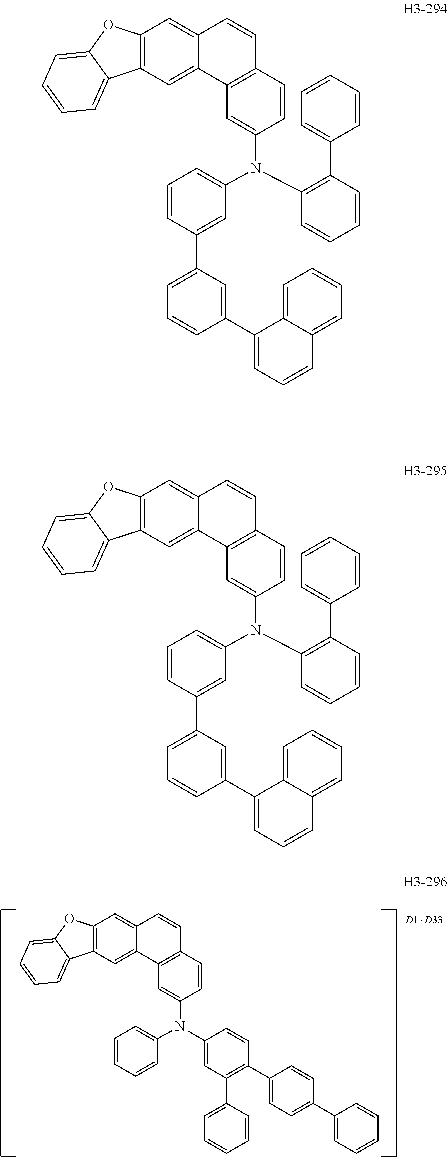

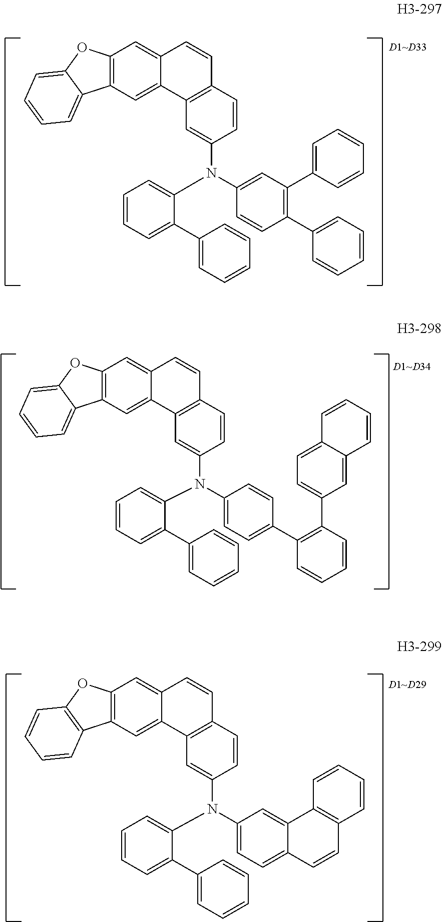

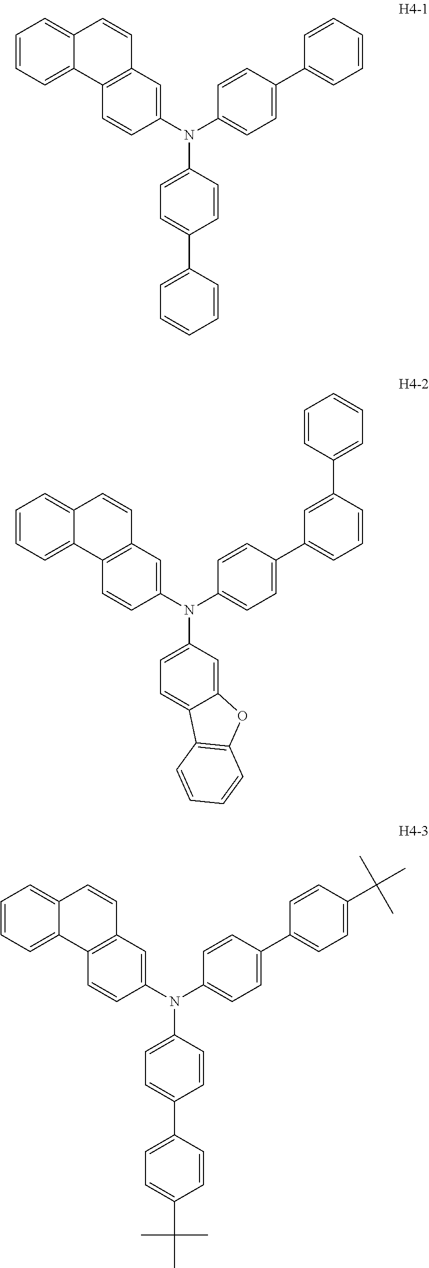 [SMILES: c1ccc(-c2ccc(N(c3ccc(-c4ccccc4)cc3)c3ccc4c(ccc5ccccc54)c3)cc2)cc1 ; c1ccc(-c2cccc(-c3ccc(N(c4ccc5c(ccc6ccccc65)c4)c4ccc5c(c4)oc4ccccc45)cc3)c2)cc1 ; CC(C)(C)c1ccc(-c2ccc(N(c3ccc(-c4ccc(C(C)(C)C)cc4)cc3)c3ccc4c(ccc5ccccc54)c3)cc2)cc1]

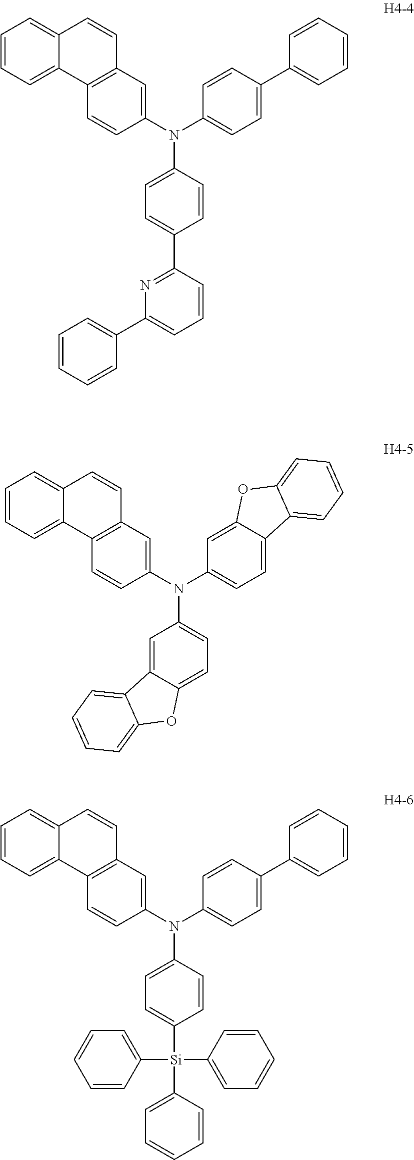 [SMILES: c1ccc(-c2ccc(N(c3ccc(-c4cccc(-c5ccccc5)n4)cc3)c3ccc4c(ccc5ccccc54)c3)cc2)cc1 ; c1ccc2c(c1)ccc1cc(N(c3ccc4c(c3)oc3ccccc34)c3ccc4oc5ccccc5c4c3)ccc12 ; c1ccc(-c2ccc(N(c3ccc([Si](c4ccccc4)(c4ccccc4)c4ccccc4)cc3)c3ccc4c(ccc5ccccc54)c3)cc2)cc1]

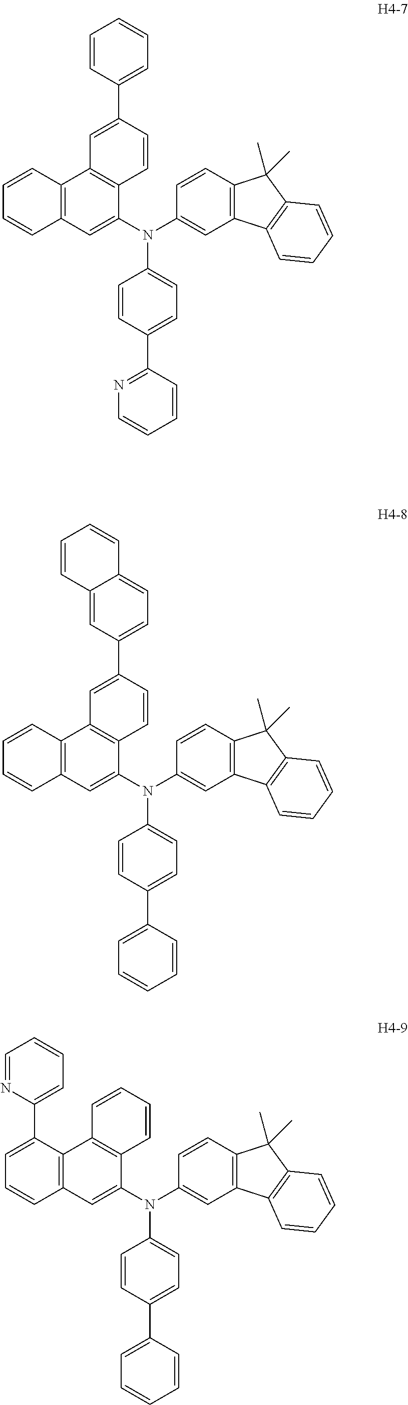 [SMILES: CC1(C)c2ccccc2-c2cc(N(c3ccc(-c4ccccn4)cc3)c3cc4ccccc4c4cc(-c5ccccc5)ccc34)ccc21 ; CC1(C)c2ccccc2-c2cc(N(c3ccc(-c4ccccc4)cc3)c3cc4ccccc4c4cc(-c5ccc6ccccc6c5)ccc34)ccc21 ; CC1(C)c2ccccc2-c2cc(N(c3ccc(-c4ccccc4)cc3)c3cc4cccc(-c5ccccn5)c4c4ccccc34)ccc21]

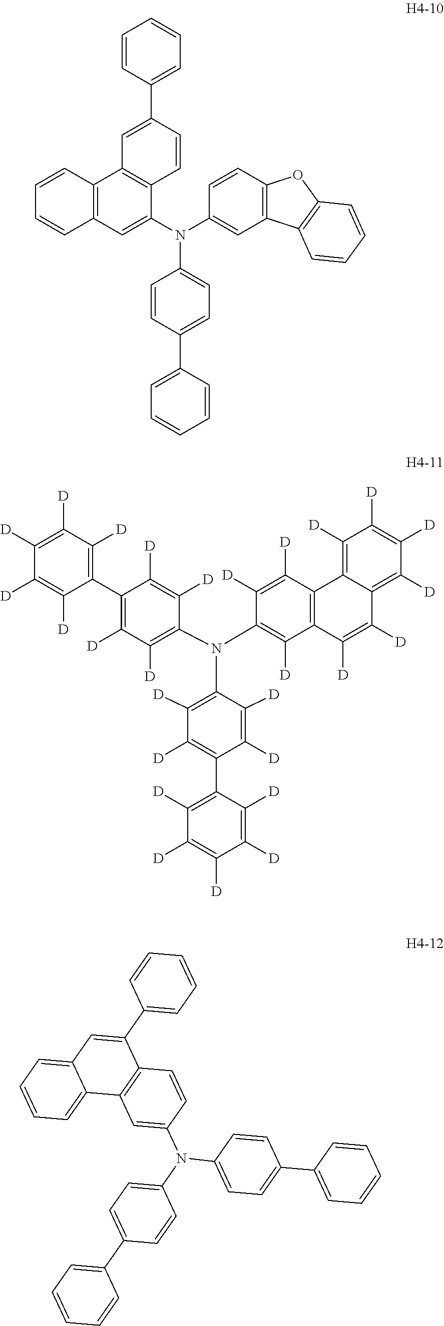 [SMILES: c1ccc(-c2ccc(N(c3ccc4oc5ccccc5c4c3)c3cc4ccccc4c4cc(-c5ccccc5)ccc34)cc2)cc1 ; [2H]c1c([2H])c([2H])c(-c2c([2H])c([2H])c(N(c3c([2H])c([2H])c(-c4c([2H])c([2H])c([2H])c([2H])c4[2H])c([2H])c3[2H])c3c([2H])c([2H])c4c(c3[2H])c([2H])c([2H])c3c([2H])c([2H])c([2H])c([2H])c34)c([2H])c2[2H])c([2H])c1[2H] ; c1ccc(-c2ccc(N(c3ccc(-c4ccccc4)cc3)c3ccc4c(-c5ccccc5)cc5ccccc5c4c3)cc2)cc1]

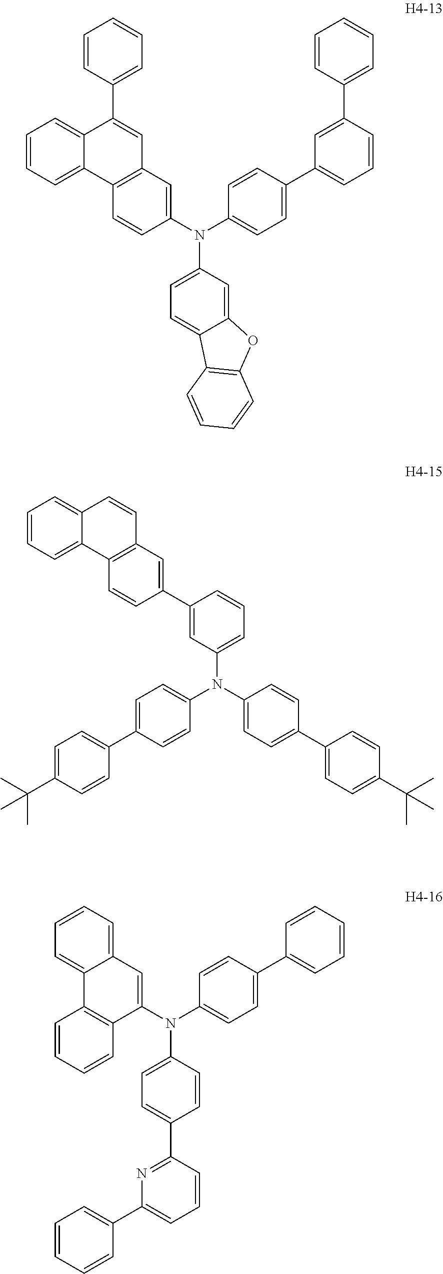 [SMILES: c1ccc(-c2cccc(-c3ccc(N(c4ccc5c(c4)cc(-c4ccccc4)c4ccccc45)c4ccc5c(c4)oc4ccccc45)cc3)c2)cc1 ; CC(C)(C)c1ccc(-c2ccc(N(c3ccc(-c4ccc(C(C)(C)C)cc4)cc3)c3cccc(-c4ccc5c(ccc6ccccc65)c4)c3)cc2)cc1 ; c1ccc(-c2ccc(N(c3ccc(-c4cccc(-c5ccccc5)n4)cc3)c3cc4ccccc4c4ccccc34)cc2)cc1]

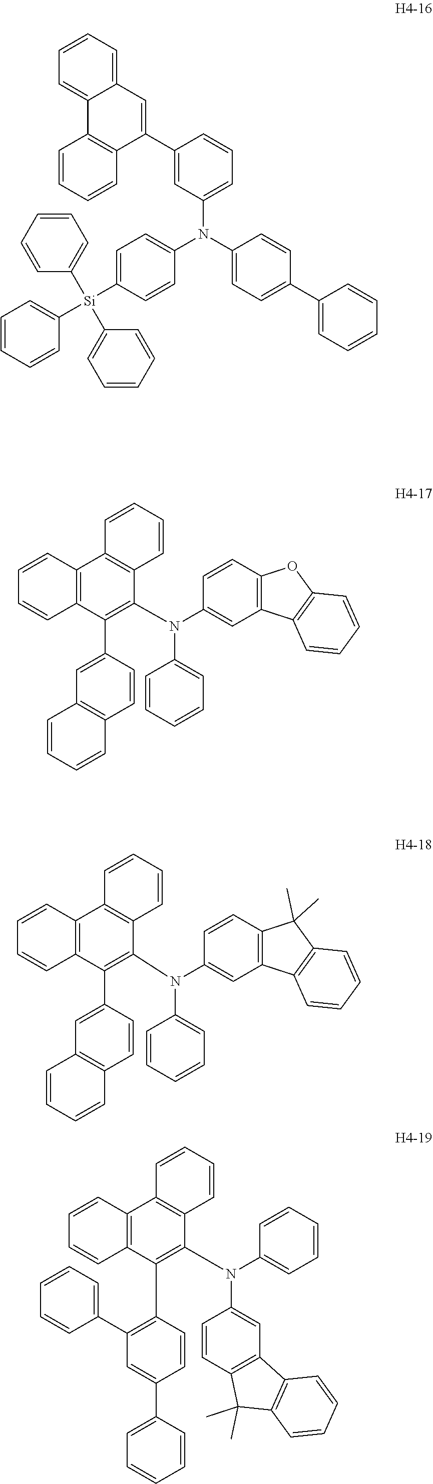 [SMILES: c1ccc(-c2ccc(N(c3ccc([Si](c4ccccc4)(c4ccccc4)c4ccccc4)cc3)c3cccc(-c4cc5ccccc5c5ccccc45)c3)cc2)cc1 ; c1ccc(N(c2ccc3oc4ccccc4c3c2)c2c(-c3ccc4ccccc4c3)c3ccccc3c3ccccc23)cc1 ; CC1(C)c2ccccc2-c2cc(N(c3ccccc3)c3c(-c4ccc5ccccc5c4)c4ccccc4c4ccccc34)ccc21 ; CC1(C)c2ccccc2-c2cc(N(c3ccccc3)c3c(-c4ccc(-c5ccccc5)cc4-c4ccccc4)c4ccccc4c4ccccc34)ccc21]

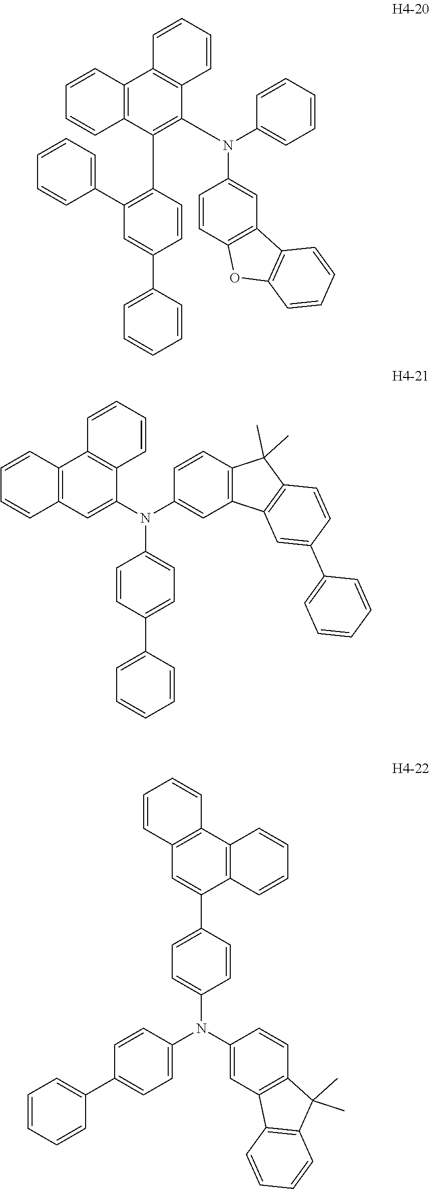 [SMILES: c1ccc(-c2ccc(-c3c(N(c4ccccc4)c4ccc5oc6ccccc6c5c4)c4ccccc4c4ccccc34)c(-c3ccccc3)c2)cc1 ; CC1(C)c2ccc(-c3ccccc3)cc2-c2cc(N(c3ccc(-c4ccccc4)cc3)c3cc4ccccc4c4ccccc34)ccc21 ; CC1(C)c2ccccc2-c2cc(N(c3ccc(-c4ccccc4)cc3)c3ccc(-c4cc5ccccc5c5ccccc45)cc3)ccc21]

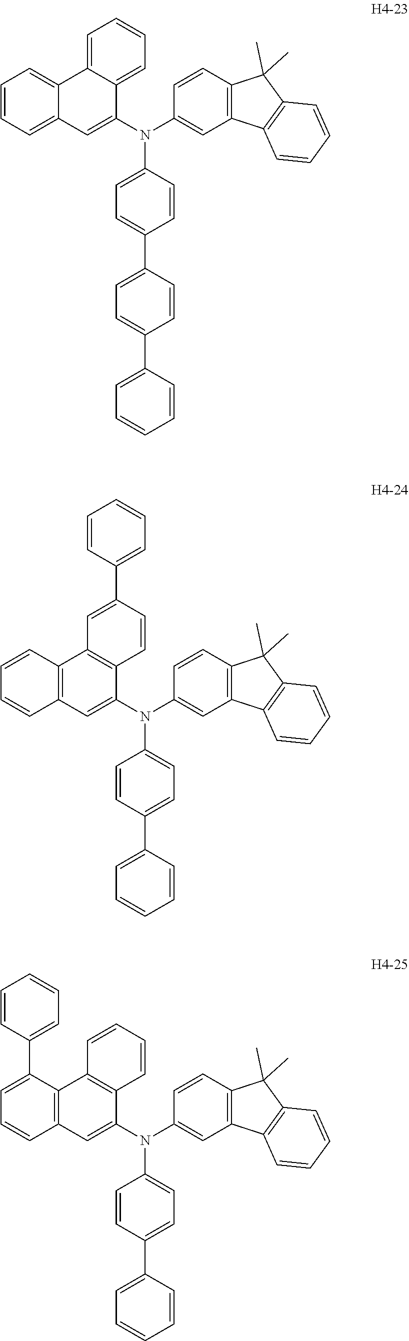 [SMILES: CC1(C)c2ccccc2-c2cc(N(c3ccc(-c4ccc(-c5ccccc5)cc4)cc3)c3cc4ccccc4c4ccccc34)ccc21 ; CC1(C)c2ccccc2-c2cc(N(c3ccc(-c4ccccc4)cc3)c3cc4ccccc4c4cc(-c5ccccc5)ccc34)ccc21 ; CC1(C)c2ccccc2-c2cc(N(c3ccc(-c4ccccc4)cc3)c3cc4cccc(-c5ccccc5)c4c4ccccc34)ccc21]

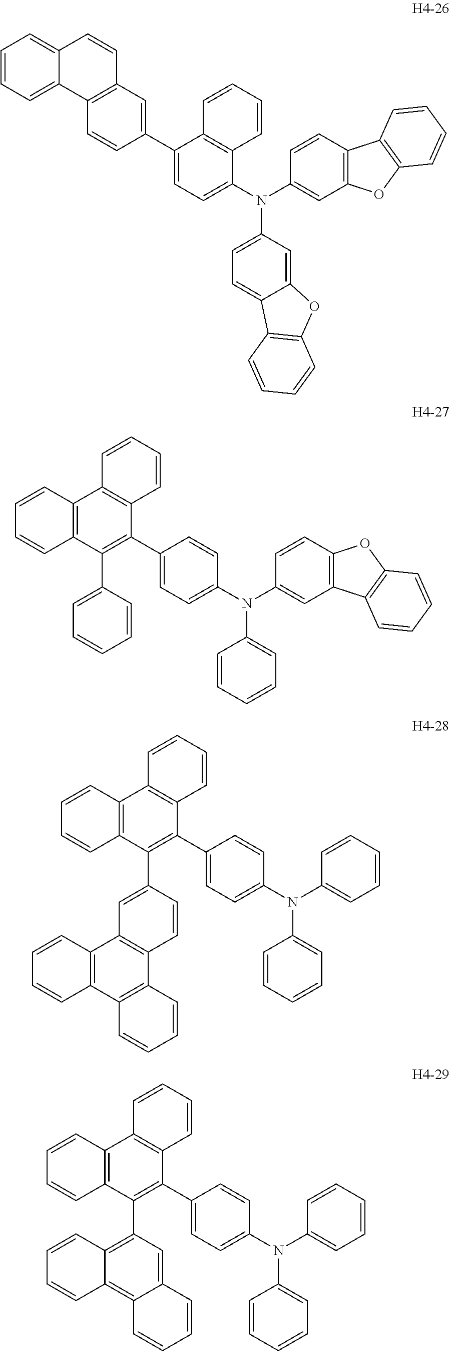

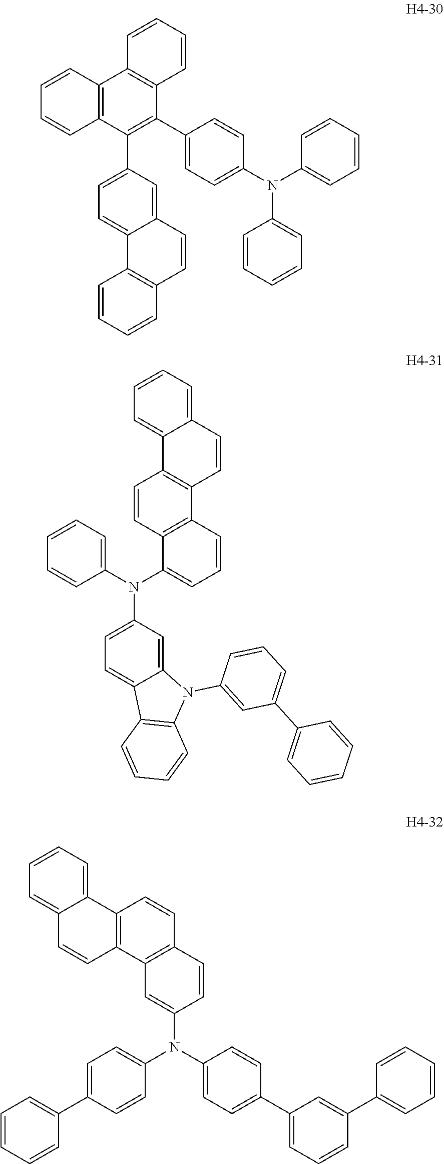 [SMILES: c1ccc(N(c2ccccc2)c2ccc(-c3c(-c4ccc5c(ccc6ccccc65)c4)c4ccccc4c4ccccc34)cc2)cc1 ; c1ccc(-c2cccc(-n3c4ccccc4c4ccc(N(c5ccccc5)c5cccc6c5ccc5c7ccccc7ccc65)cc43)c2)cc1 ; c1ccc(-c2ccc(N(c3ccc(-c4cccc(-c5ccccc5)c4)cc3)c3ccc4ccc5c6ccccc6ccc5c4c3)cc2)cc1]

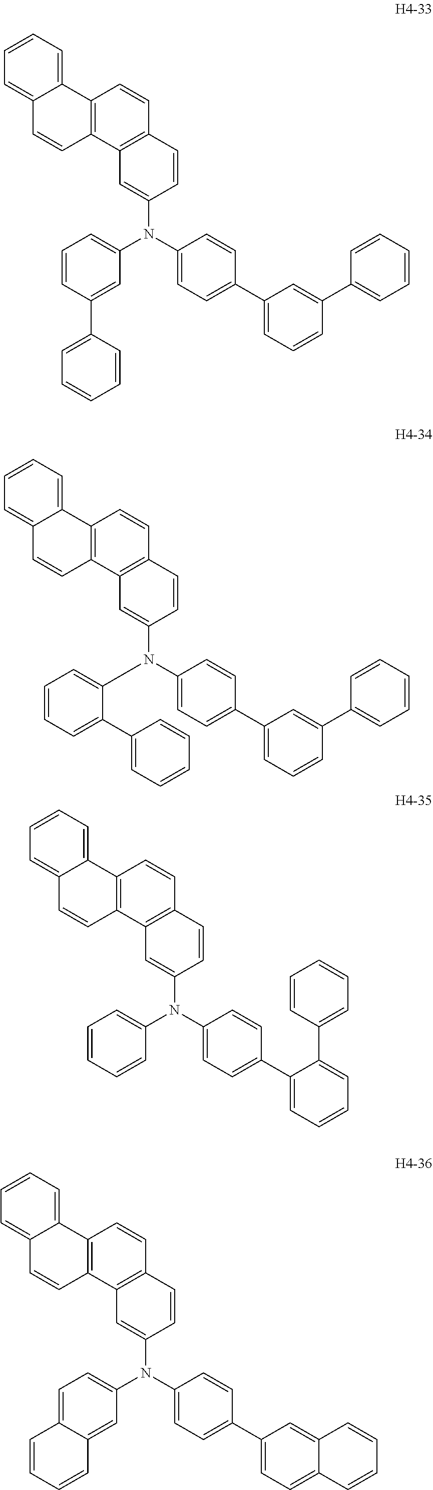 [SMILES: c1ccc(-c2cccc(-c3ccc(N(c4cccc(-c5ccccc5)c4)c4ccc5ccc6c7ccccc7ccc6c5c4)cc3)c2)cc1 ; c1ccc(-c2cccc(-c3ccc(N(c4ccc5ccc6c7ccccc7ccc6c5c4)c4ccccc4-c4ccccc4)cc3)c2)cc1 ; c1ccc(-c2ccccc2-c2ccc(N(c3ccccc3)c3ccc4ccc5c6ccccc6ccc5c4c3)cc2)cc1 ; c1ccc2cc(-c3ccc(N(c4ccc5ccccc5c4)c4ccc5ccc6c7ccccc7ccc6c5c4)cc3)ccc2c1]

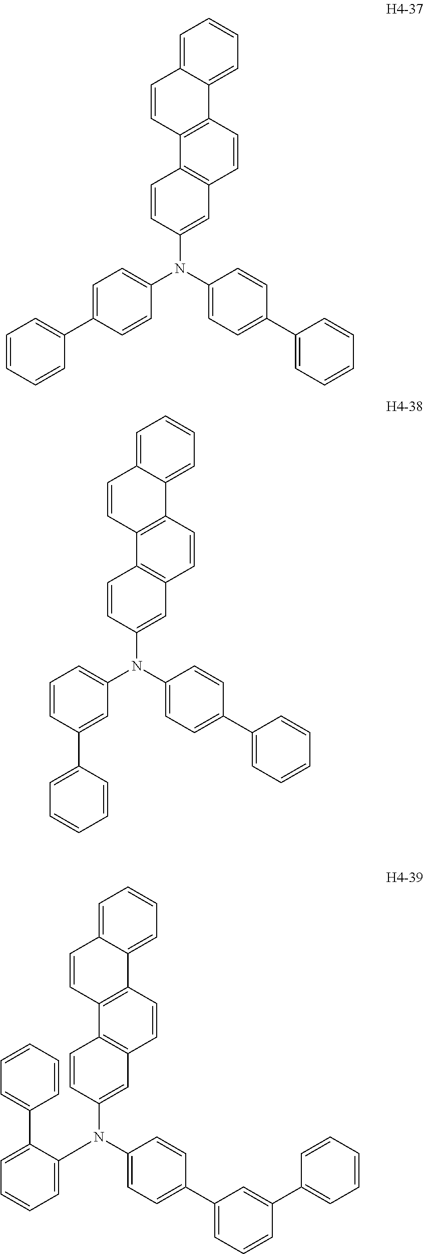 [SMILES: c1ccc(-c2ccc(N(c3ccc(-c4ccccc4)cc3)c3ccc4c(ccc5c6ccccc6ccc45)c3)cc2)cc1 ; c1ccc(-c2ccc(N(c3cccc(-c4ccccc4)c3)c3ccc4c(ccc5c6ccccc6ccc45)c3)cc2)cc1 ; c1ccc(-c2cccc(-c3ccc(N(c4ccc5c(ccc6c7ccccc7ccc56)c4)c4ccccc4-c4ccccc4)cc3)c2)cc1]

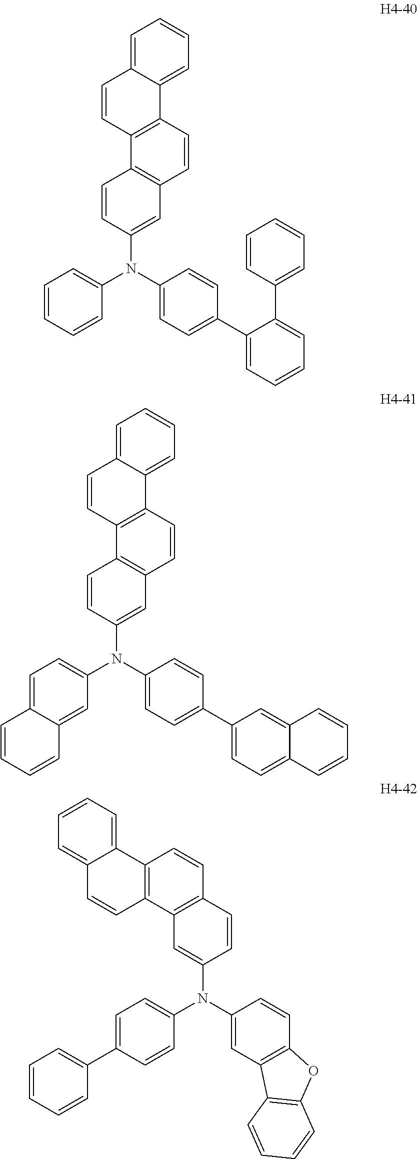 [SMILES: c1ccc(-c2ccccc2-c2ccc(N(c3ccccc3)c3ccc4c(ccc5c6ccccc6ccc45)c3)cc2)cc1 ; c1ccc2cc(-c3ccc(N(c4ccc5ccccc5c4)c4ccc5c(ccc6c7ccccc7ccc56)c4)cc3)ccc2c1 ; c1ccc(-c2ccc(N(c3ccc4ccc5c6ccccc6ccc5c4c3)c3ccc4oc5ccccc5c4c3)cc2)cc1]

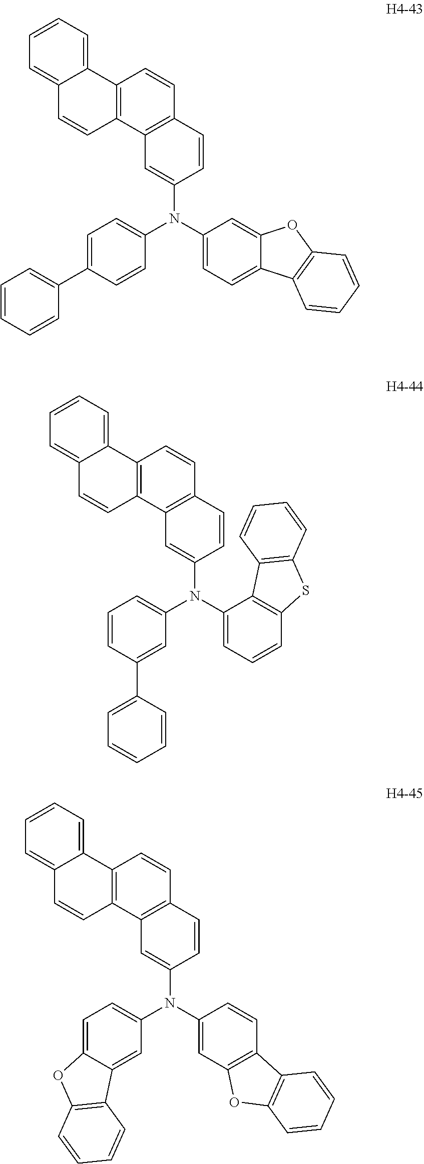 [SMILES: c1ccc(-c2ccc(N(c3ccc4c(c3)oc3ccccc34)c3ccc4ccc5c6ccccc6ccc5c4c3)cc2)cc1 ; c1ccc(-c2cccc(N(c3ccc4ccc5c6ccccc6ccc5c4c3)c3cccc4sc5ccccc5c34)c2)cc1 ; c1ccc2c(c1)ccc1c3cc(N(c4ccc5c(c4)oc4ccccc45)c4ccc5oc6ccccc6c5c4)ccc3ccc21]

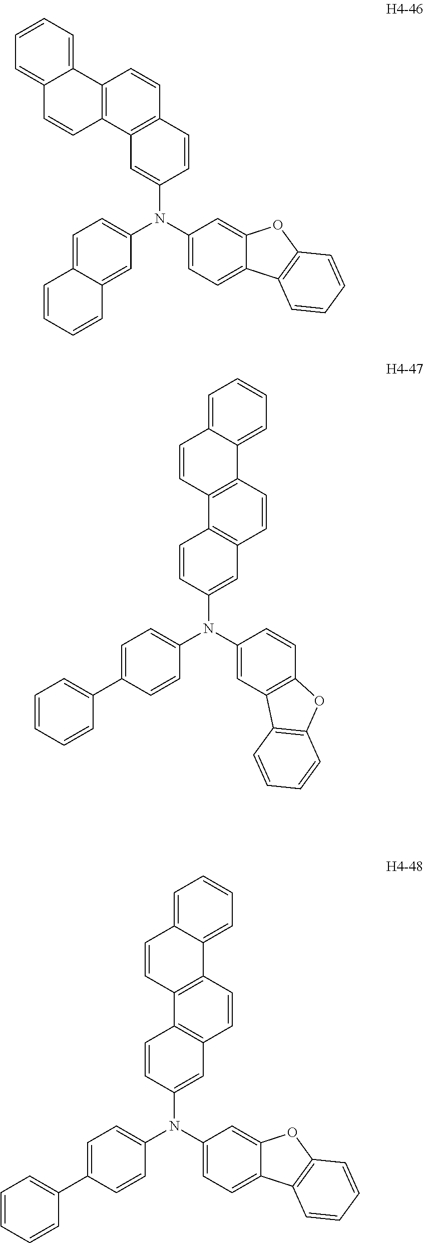 [SMILES: c1ccc2cc(N(c3ccc4c(c3)oc3ccccc34)c3ccc4ccc5c6ccccc6ccc5c4c3)ccc2c1 ; c1ccc(-c2ccc(N(c3ccc4c(ccc5c6ccccc6ccc45)c3)c3ccc4oc5ccccc5c4c3)cc2)cc1 ; c1ccc(-c2ccc(N(c3ccc4c(ccc5c6ccccc6ccc45)c3)c3ccc4c(c3)oc3ccccc34)cc2)cc1]

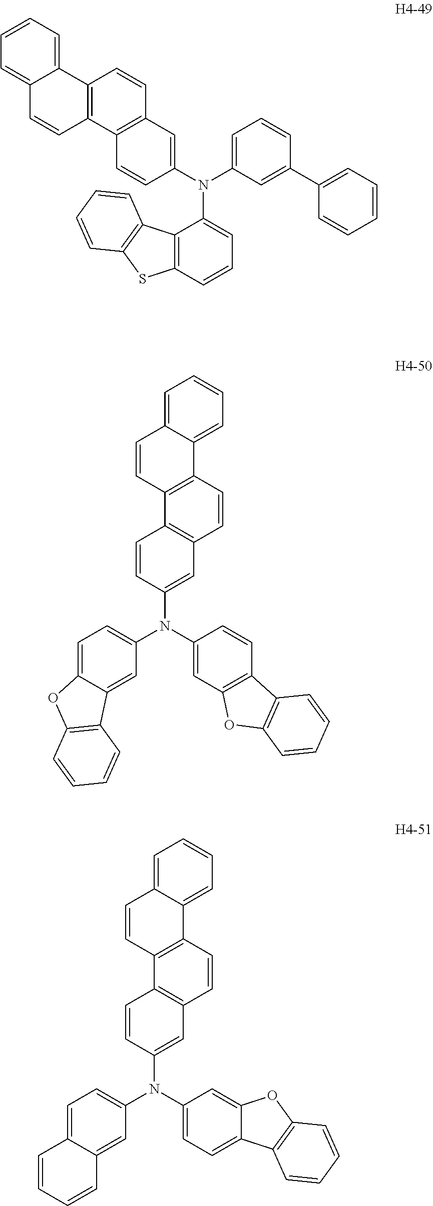 [SMILES: c1ccc(-c2cccc(N(c3ccc4c(ccc5c6ccccc6ccc45)c3)c3cccc4sc5ccccc5c34)c2)cc1 ; c1ccc2c(c1)ccc1c3ccc(N(c4ccc5c(c4)oc4ccccc45)c4ccc5oc6ccccc6c5c4)cc3ccc21 ; c1ccc2cc(N(c3ccc4c(ccc5c6ccccc6ccc45)c3)c3ccc4c(c3)oc3ccccc34)ccc2c1]

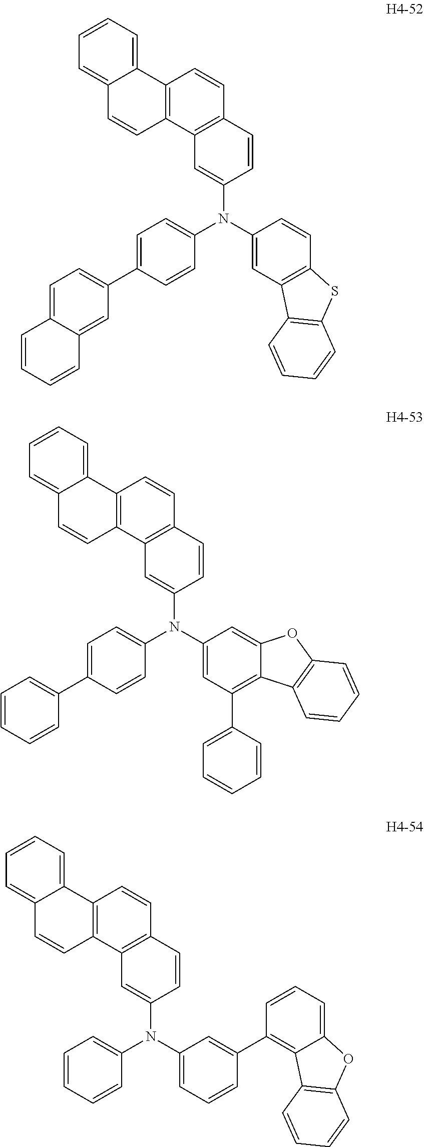

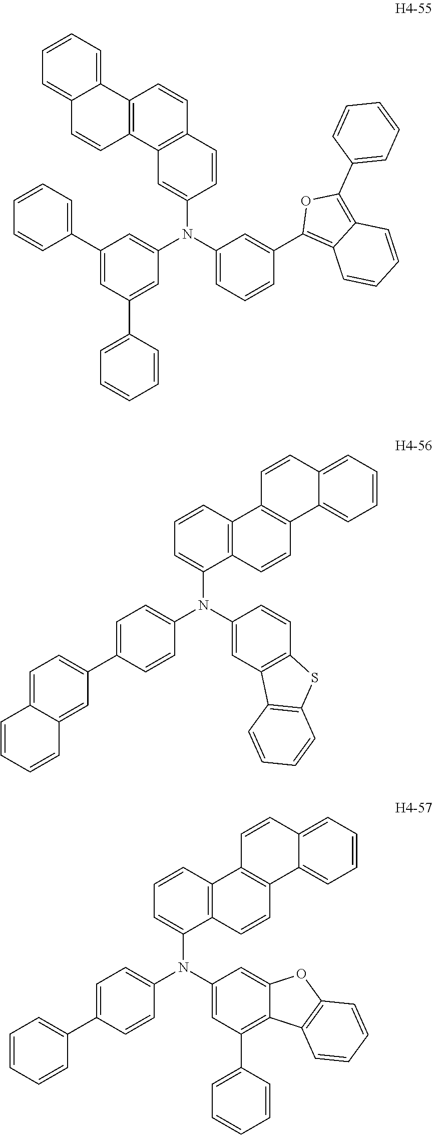 [SMILES: c1ccc(-c2cc(-c3ccccc3)cc(N(c3cccc(-c4oc(-c5ccccc5)c5ccccc45)c3)c3ccc4ccc5c6ccccc6ccc5c4c3)c2)cc1 ; c1ccc2cc(-c3ccc(N(c4ccc5sc6ccccc6c5c4)c4cccc5c4ccc4c6ccccc6ccc54)cc3)ccc2c1 ; c1ccc(-c2ccc(N(c3cc(-c4ccccc4)c4c(c3)oc3ccccc34)c3cccc4c3ccc3c5ccccc5ccc43)cc2)cc1]

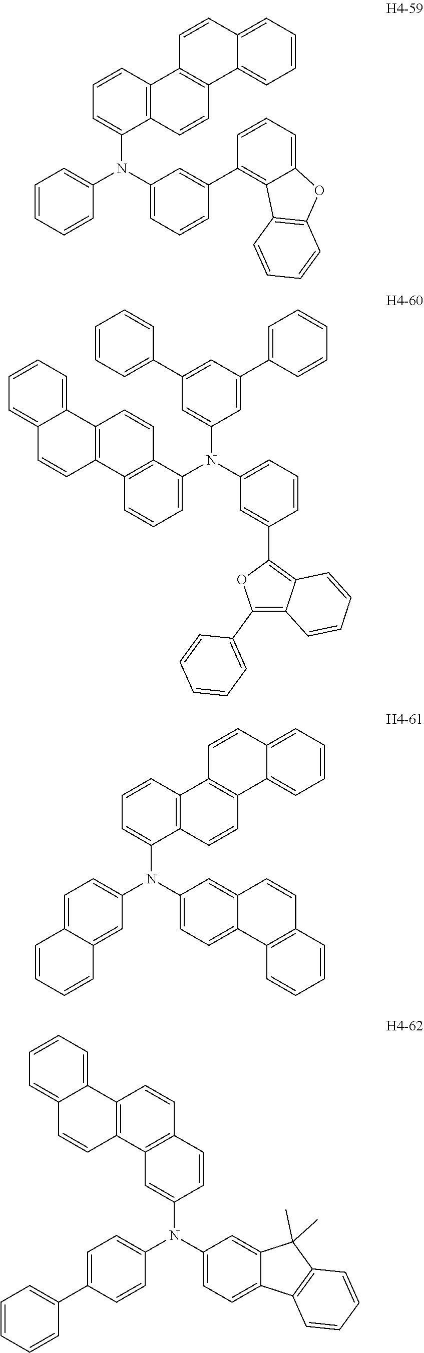

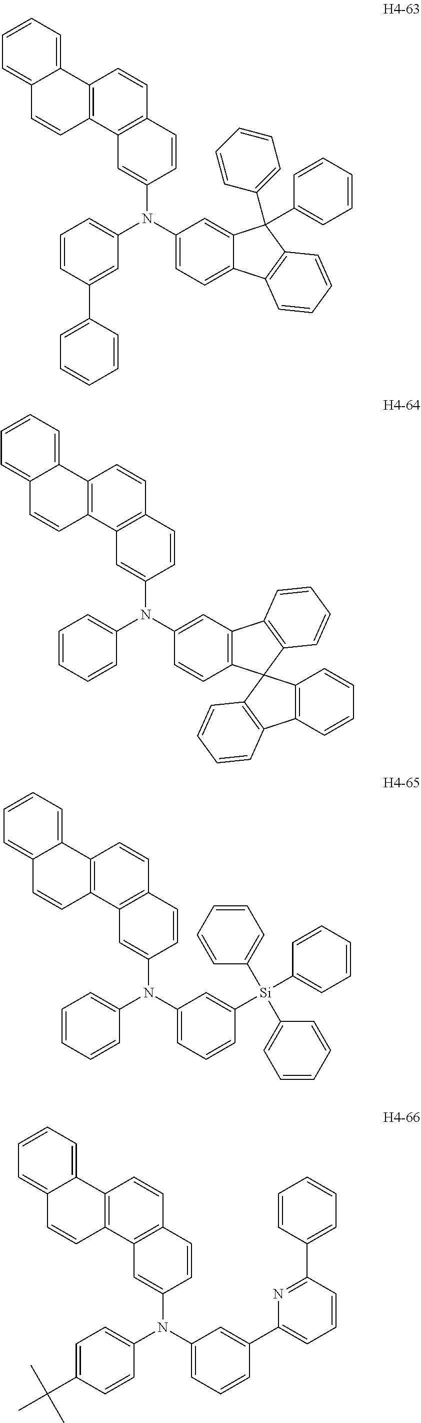 [SMILES: c1ccc(-c2cccc(N(c3ccc4c(c3)C(c3ccccc3)(c3ccccc3)c3ccccc3-4)c3ccc4ccc5c6ccccc6ccc5c4c3)c2)cc1 ; c1ccc(N(c2ccc3c(c2)-c2ccccc2C32c3ccccc3-c3ccccc32)c2ccc3ccc4c5ccccc5ccc4c3c2)cc1 ; c1ccc(N(c2cccc([Si](c3ccccc3)(c3ccccc3)c3ccccc3)c2)c2ccc3ccc4c5ccccc5ccc4c3c2)cc1 ; CC(C)(C)c1ccc(N(c2cccc(-c3cccc(-c4ccccc4)n3)c2)c2ccc3ccc4c5ccccc5ccc4c3c2)cc1]

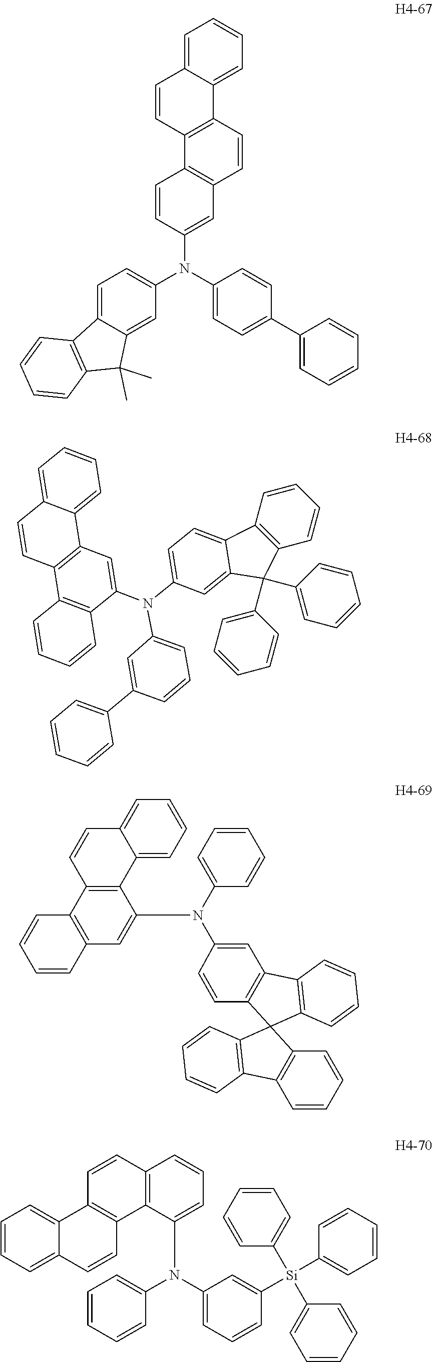 [SMILES: CC1(C)c2ccccc2-c2ccc(N(c3ccc(-c4ccccc4)cc3)c3ccc4c(ccc5c6ccccc6ccc45)c3)cc21 ; c1ccc(-c2cccc(N(c3ccc4c(c3)C(c3ccccc3)(c3ccccc3)c3ccccc3-4)c3cc4c5ccccc5ccc4c4ccccc34)c2)cc1 ; c1ccc(N(c2ccc3c(c2)-c2ccccc2C32c3ccccc3-c3ccccc32)c2cc3ccccc3c3ccc4ccccc4c23)cc1 ; c1ccc(N(c2cccc([Si](c3ccccc3)(c3ccccc3)c3ccccc3)c2)c2cccc3ccc4c5ccccc5ccc4c23)cc1]

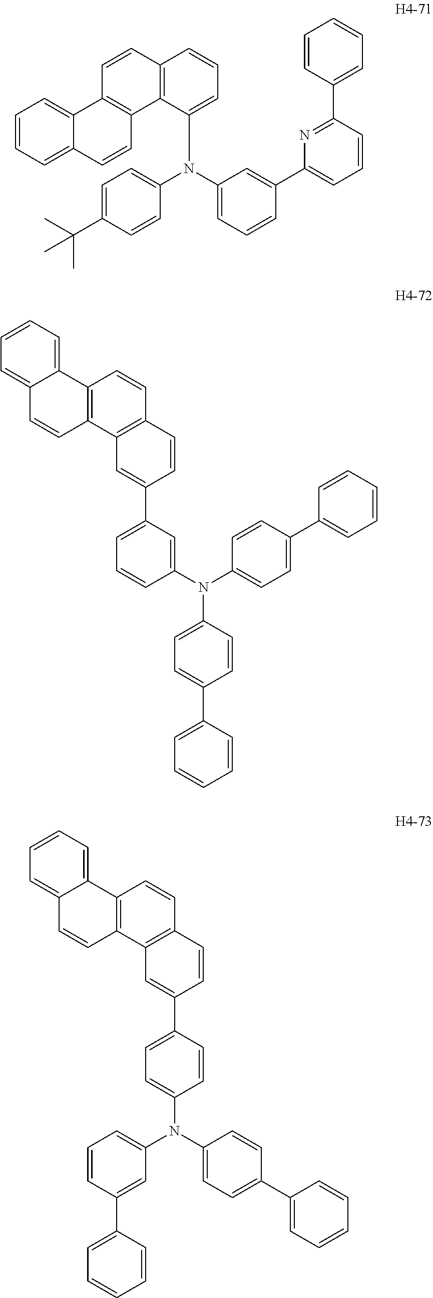 [SMILES: CC(C)(C)c1ccc(N(c2cccc(-c3cccc(-c4ccccc4)n3)c2)c2cccc3ccc4c5ccccc5ccc4c23)cc1 ; c1ccc(-c2ccc(N(c3ccc(-c4ccccc4)cc3)c3cccc(-c4ccc5ccc6c7ccccc7ccc6c5c4)c3)cc2)cc1 ; c1ccc(-c2ccc(N(c3ccc(-c4ccc5ccc6c7ccccc7ccc6c5c4)cc3)c3cccc(-c4ccccc4)c3)cc2)cc1]

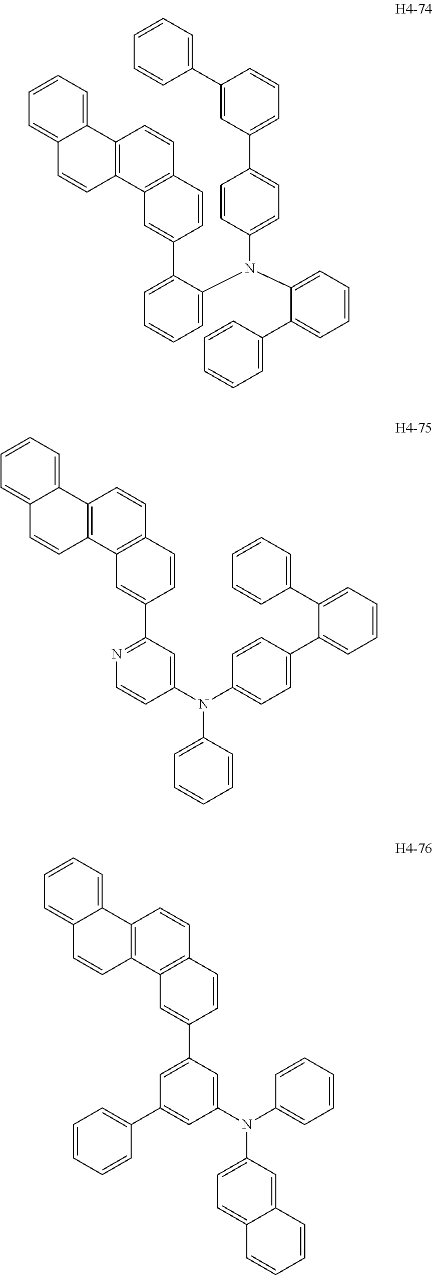 [SMILES: c1ccc(-c2cccc(-c3ccc(N(c4ccccc4-c4ccccc4)c4ccccc4-c4ccc5ccc6c7ccccc7ccc6c5c4)cc3)c2)cc1 ; c1ccc(-c2ccccc2-c2ccc(N(c3ccccc3)c3ccnc(-c4ccc5ccc6c7ccccc7ccc6c5c4)c3)cc2)cc1 ; c1ccc(-c2cc(-c3ccc4ccc5c6ccccc6ccc5c4c3)cc(N(c3ccccc3)c3ccc4ccccc4c3)c2)cc1]

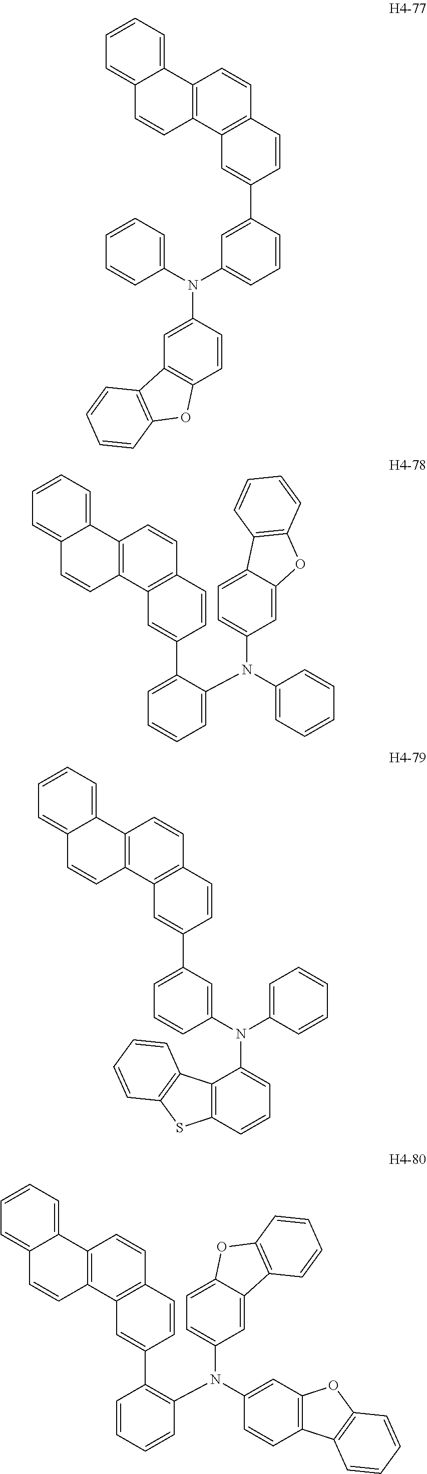 [SMILES: c1ccc(N(c2cccc(-c3ccc4ccc5c6ccccc6ccc5c4c3)c2)c2ccc3oc4ccccc4c3c2)cc1 ; c1ccc(N(c2ccc3c(c2)oc2ccccc23)c2ccccc2-c2ccc3ccc4c5ccccc5ccc4c3c2)cc1 ; c1ccc(N(c2cccc(-c3ccc4ccc5c6ccccc6ccc5c4c3)c2)c2cccc3sc4ccccc4c23)cc1 ; c1ccc(N(c2ccc3c(c2)oc2ccccc23)c2ccc3oc4ccccc4c3c2)c(-c2ccc3ccc4c5ccccc5ccc4c3c2)c1]

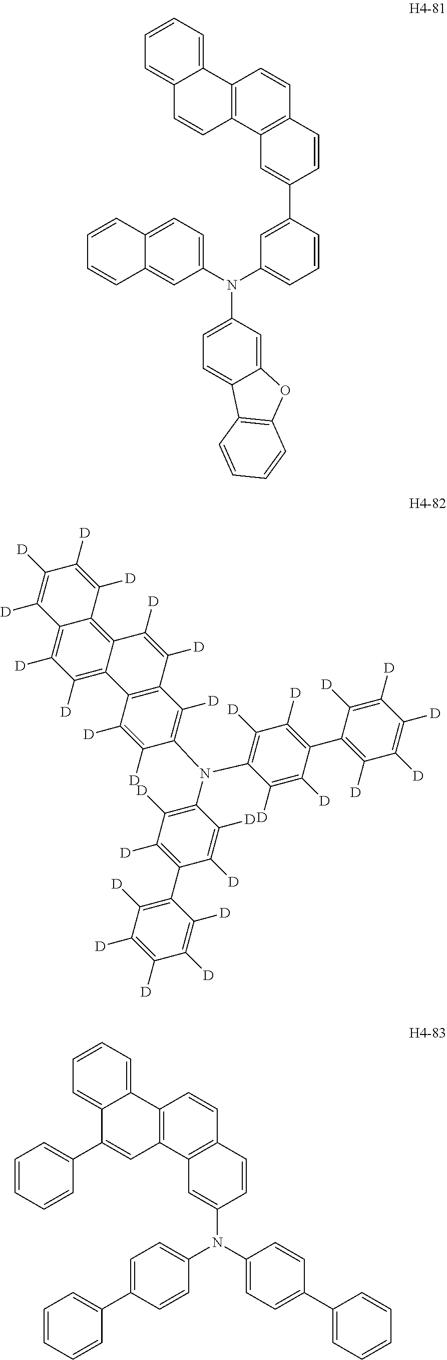 [SMILES: c1cc(-c2ccc3ccc4c5ccccc5ccc4c3c2)cc(N(c2ccc3ccccc3c2)c2ccc3c(c2)oc2ccccc23)c1 ; [2H]c1c([2H])c([2H])c(-c2c([2H])c([2H])c(N(c3c([2H])c([2H])c(-c4c([2H])c([2H])c([2H])c([2H])c4[2H])c([2H])c3[2H])c3c([2H])c([2H])c4c(c3[2H])c([2H])c([2H])c3c5c([2H])c([2H])c([2H])c([2H])c5c([2H])c([2H])c43)c([2H])c2[2H])c([2H])c1[2H] ; c1ccc(-c2ccc(N(c3ccc(-c4ccccc4)cc3)c3ccc4ccc5c6ccccc6c(-c6ccccc6)cc5c4c3)cc2)cc1]

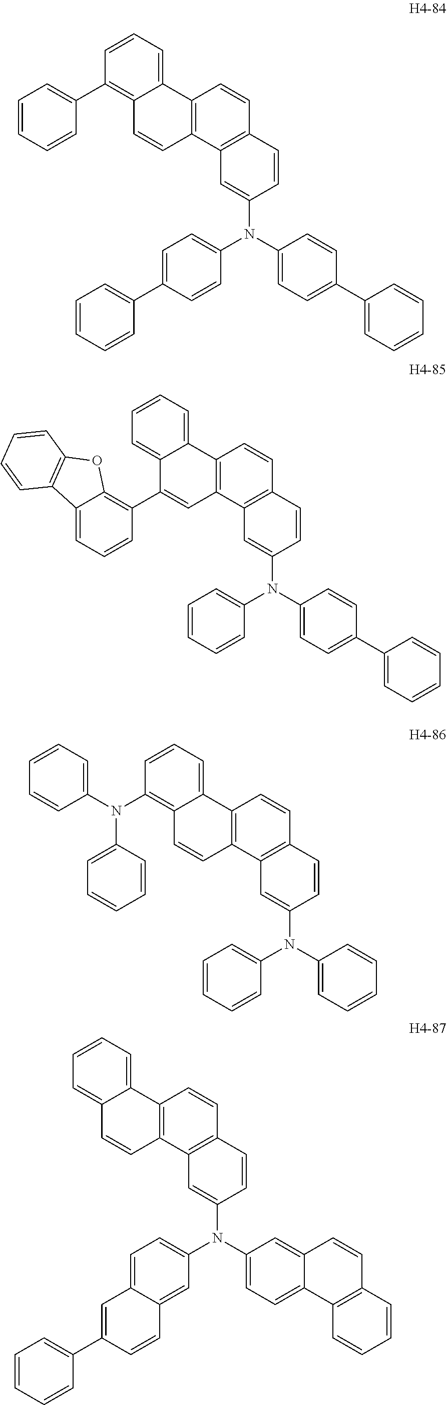 [SMILES: c1ccc(-c2ccc(N(c3ccc(-c4ccccc4)cc3)c3ccc4ccc5c6cccc(-c7ccccc7)c6ccc5c4c3)cc2)cc1 ; c1ccc(-c2ccc(N(c3ccccc3)c3ccc4ccc5c6ccccc6c(-c6cccc7c6oc6ccccc67)cc5c4c3)cc2)cc1 ; c1ccc(N(c2ccccc2)c2ccc3ccc4c5cccc(N(c6ccccc6)c6ccccc6)c5ccc4c3c2)cc1 ; c1ccc(-c2ccc3cc(N(c4ccc5c(ccc6ccccc65)c4)c4ccc5ccc6c7ccccc7ccc6c5c4)ccc3c2)cc1]

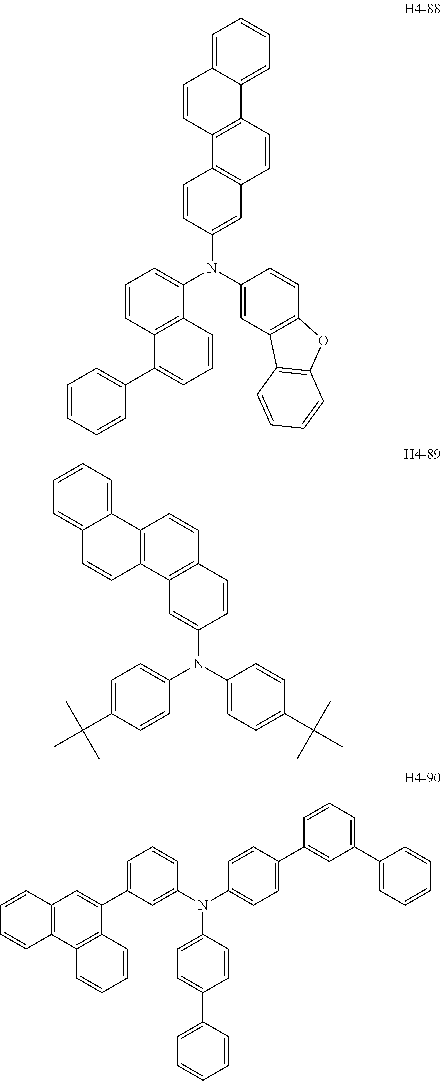

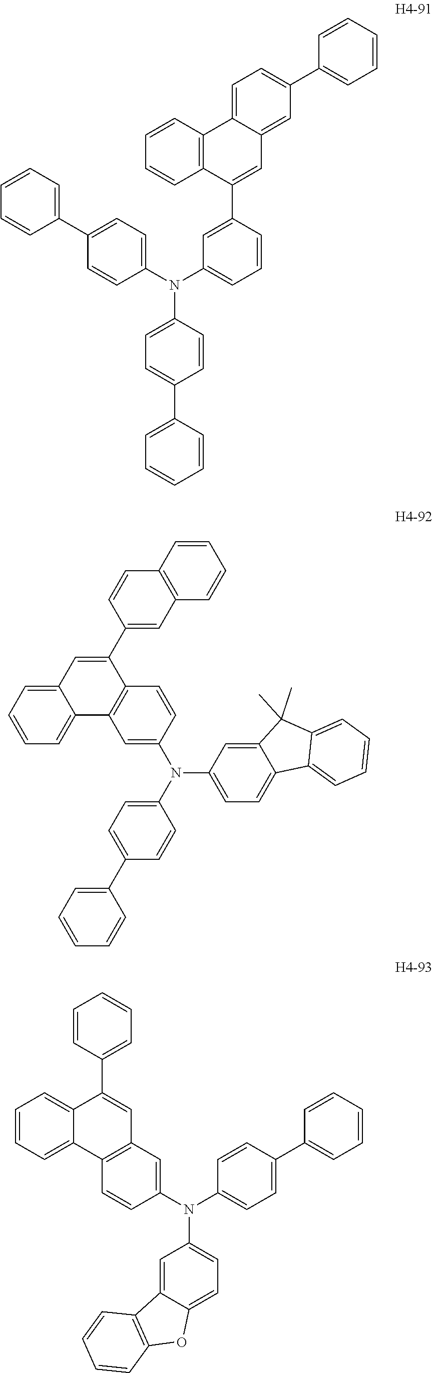 [SMILES: c1ccc(-c2ccc(N(c3ccc(-c4ccccc4)cc3)c3cccc(-c4cc5cc(-c6ccccc6)ccc5c5ccccc45)c3)cc2)cc1 ; CC1(C)c2ccccc2-c2ccc(N(c3ccc(-c4ccccc4)cc3)c3ccc4c(-c5ccc6ccccc6c5)cc5ccccc5c4c3)cc21 ; c1ccc(-c2ccc(N(c3ccc4c(c3)cc(-c3ccccc3)c3ccccc34)c3ccc4oc5ccccc5c4c3)cc2)cc1]

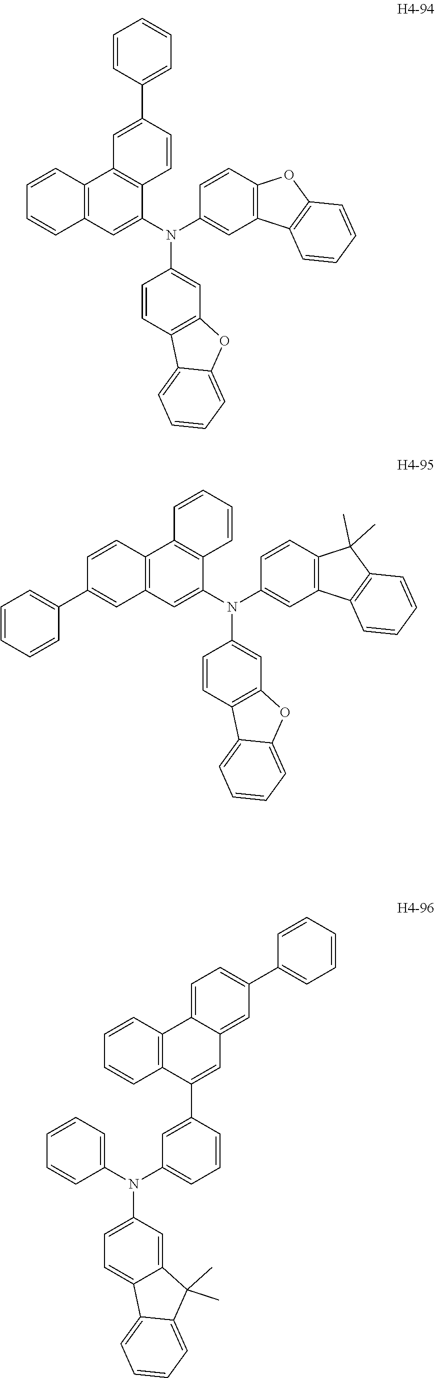 [SMILES: c1ccc(-c2ccc3c(N(c4ccc5c(c4)oc4ccccc45)c4ccc5oc6ccccc6c5c4)cc4ccccc4c3c2)cc1 ; CC1(C)c2ccccc2-c2cc(N(c3ccc4c(c3)oc3ccccc34)c3cc4cc(-c5ccccc5)ccc4c4ccccc34)ccc21 ; CC1(C)c2ccccc2-c2ccc(N(c3ccccc3)c3cccc(-c4cc5cc(-c6ccccc6)ccc5c5ccccc45)c3)cc21]

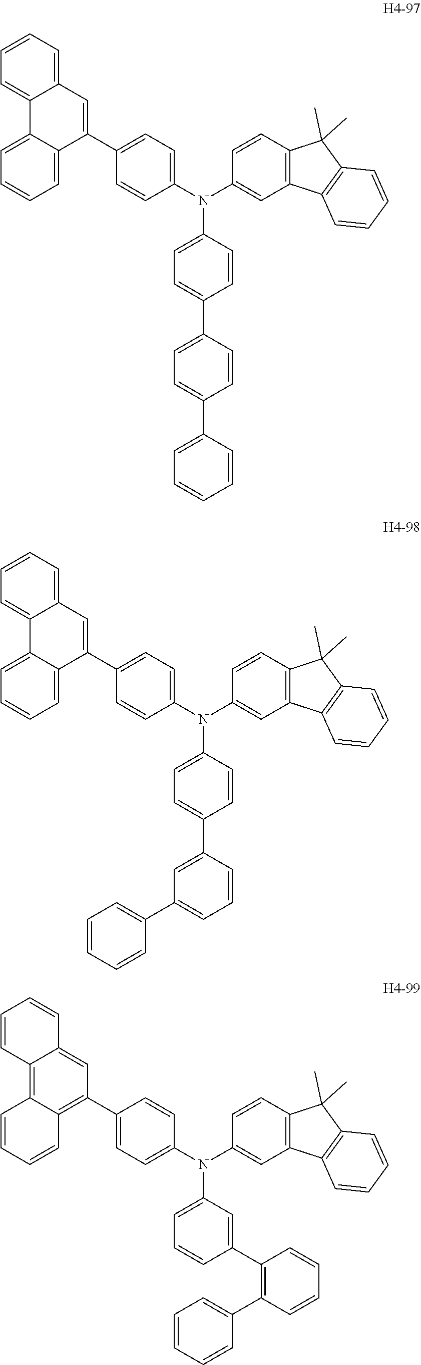 [SMILES: CC1(C)c2ccccc2-c2cc(N(c3ccc(-c4ccc(-c5ccccc5)cc4)cc3)c3ccc(-c4cc5ccccc5c5ccccc45)cc3)ccc21 ; CC1(C)c2ccccc2-c2cc(N(c3ccc(-c4cccc(-c5ccccc5)c4)cc3)c3ccc(-c4cc5ccccc5c5ccccc45)cc3)ccc21 ; CC1(C)c2ccccc2-c2cc(N(c3ccc(-c4cc5ccccc5c5ccccc45)cc3)c3cccc(-c4ccccc4-c4ccccc4)c3)ccc21]

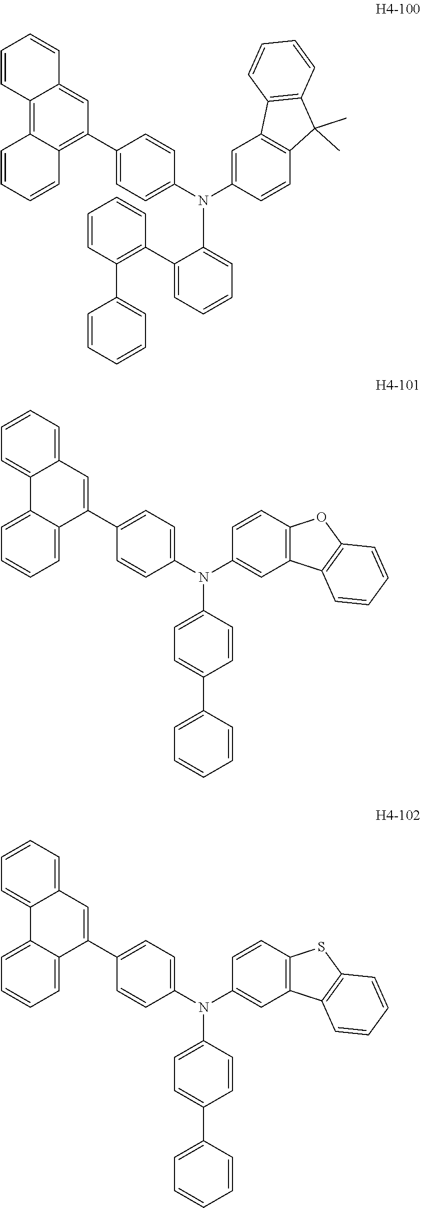 [SMILES: CC1(C)c2ccccc2-c2cc(N(c3ccc(-c4cc5ccccc5c5ccccc45)cc3)c3ccccc3-c3ccccc3-c3ccccc3)ccc21 ; c1ccc(-c2ccc(N(c3ccc(-c4cc5ccccc5c5ccccc45)cc3)c3ccc4oc5ccccc5c4c3)cc2)cc1 ; c1ccc(-c2ccc(N(c3ccc(-c4cc5ccccc5c5ccccc45)cc3)c3ccc4sc5ccccc5c4c3)cc2)cc1]

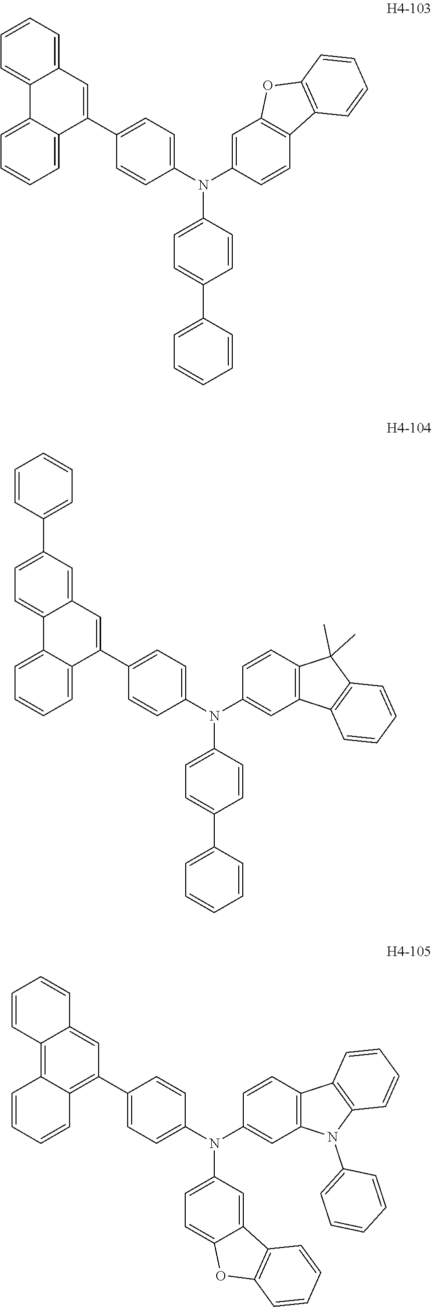 [SMILES: c1ccc(-c2ccc(N(c3ccc(-c4cc5ccccc5c5ccccc45)cc3)c3ccc4c(c3)oc3ccccc34)cc2)cc1 ; CC1(C)c2ccccc2-c2cc(N(c3ccc(-c4ccccc4)cc3)c3ccc(-c4cc5cc(-c6ccccc6)ccc5c5ccccc45)cc3)ccc21 ; c1ccc(-n2c3ccccc3c3ccc(N(c4ccc(-c5cc6ccccc6c6ccccc56)cc4)c4ccc5oc6ccccc6c5c4)cc32)cc1]

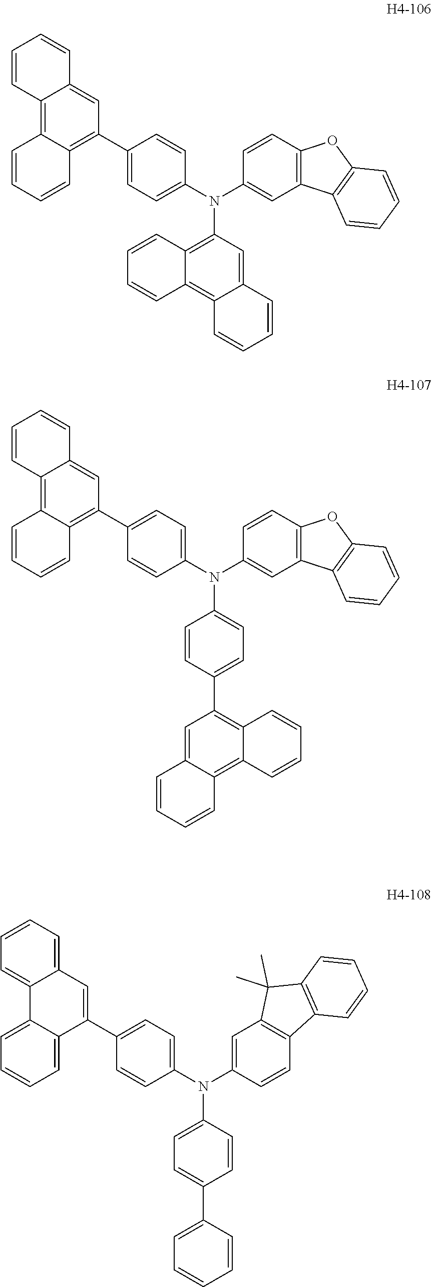 [SMILES: c1ccc2c(c1)cc(-c1ccc(N(c3ccc4oc5ccccc5c4c3)c3cc4ccccc4c4ccccc34)cc1)c1ccccc12 ; c1ccc2c(c1)cc(-c1ccc(N(c3ccc(-c4cc5ccccc5c5ccccc45)cc3)c3ccc4oc5ccccc5c4c3)cc1)c1ccccc12 ; CC1(C)c2ccccc2-c2ccc(N(c3ccc(-c4ccccc4)cc3)c3ccc(-c4cc5ccccc5c5ccccc45)cc3)cc21]

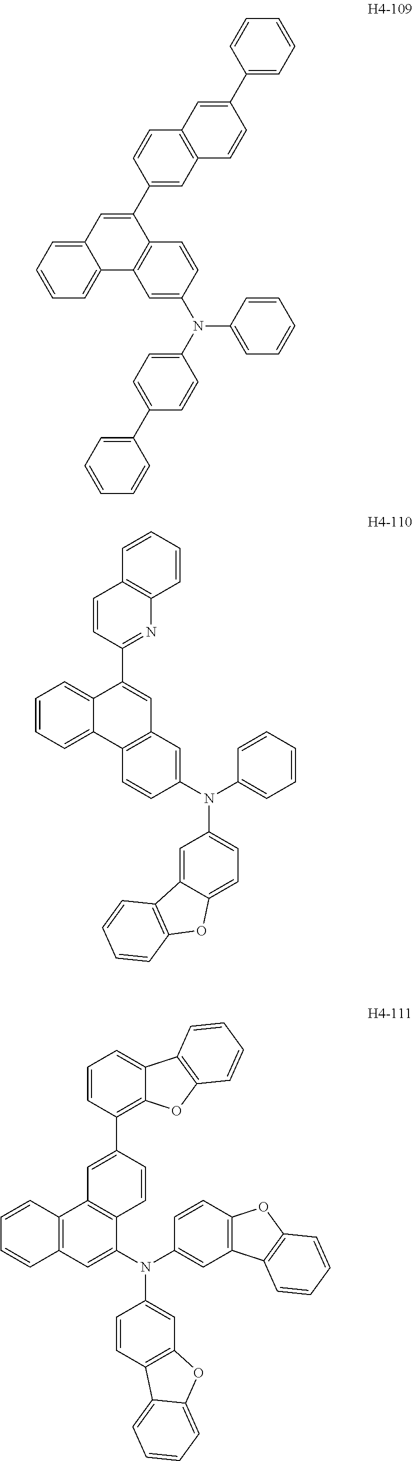

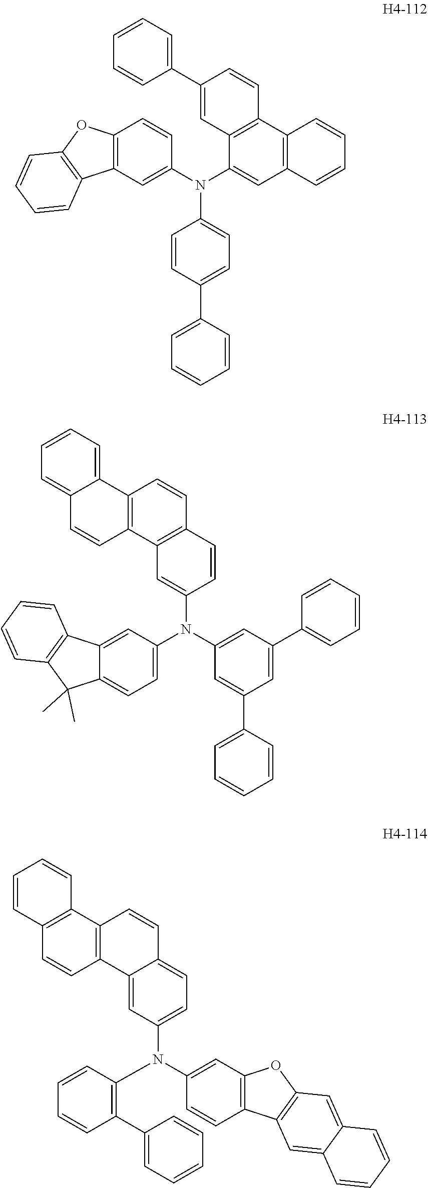 [SMILES: c1ccc(-c2ccc(N(c3ccc4oc5ccccc5c4c3)c3cc4ccccc4c4ccc(-c5ccccc5)cc34)cc2)cc1 ; CC1(C)c2ccccc2-c2cc(N(c3cc(-c4ccccc4)cc(-c4ccccc4)c3)c3ccc4ccc5c6ccccc6ccc5c4c3)ccc21 ; c1ccc(-c2ccccc2N(c2ccc3c(c2)oc2cc4ccccc4cc23)c2ccc3ccc4c5ccccc5ccc4c3c2)cc1]

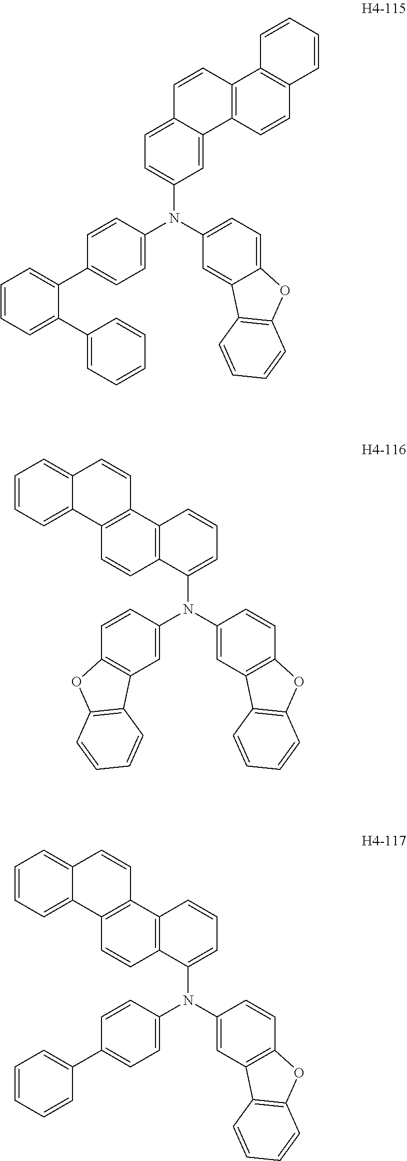 [SMILES: c1ccc(-c2ccccc2-c2ccc(N(c3ccc4ccc5c6ccccc6ccc5c4c3)c3ccc4oc5ccccc5c4c3)cc2)cc1 ; c1ccc2c(c1)ccc1c3cccc(N(c4ccc5oc6ccccc6c5c4)c4ccc5oc6ccccc6c5c4)c3ccc21 ; c1ccc(-c2ccc(N(c3ccc4oc5ccccc5c4c3)c3cccc4c3ccc3c5ccccc5ccc43)cc2)cc1]

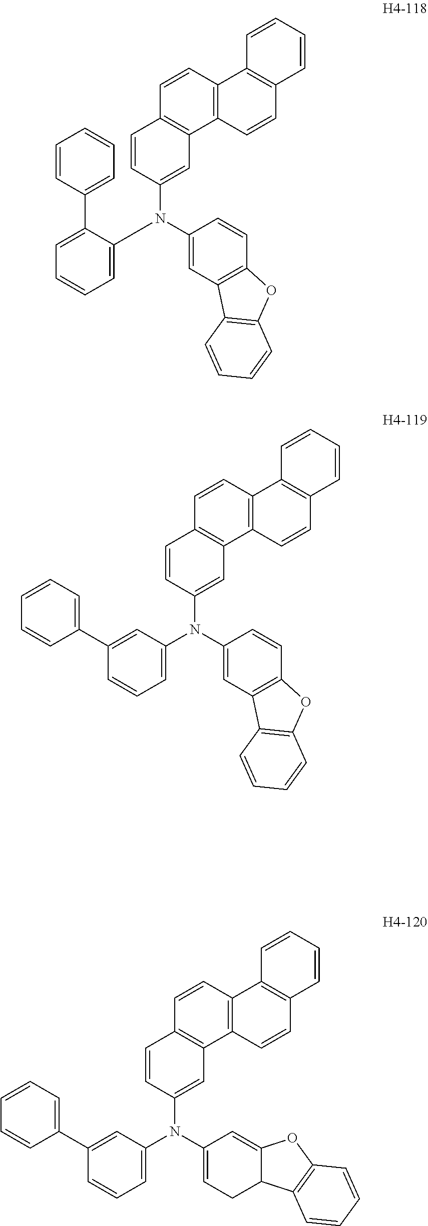 [SMILES: c1ccc(-c2ccccc2N(c2ccc3ccc4c5ccccc5ccc4c3c2)c2ccc3oc4ccccc4c3c2)cc1 ; c1ccc(-c2cccc(N(c3ccc4ccc5c6ccccc6ccc5c4c3)c3ccc4oc5ccccc5c4c3)c2)cc1 ; C1=C2Oc3ccccc3C2CC=C1N(c1cccc(-c2ccccc2)c1)c1ccc2ccc3c4ccccc4ccc3c2c1]

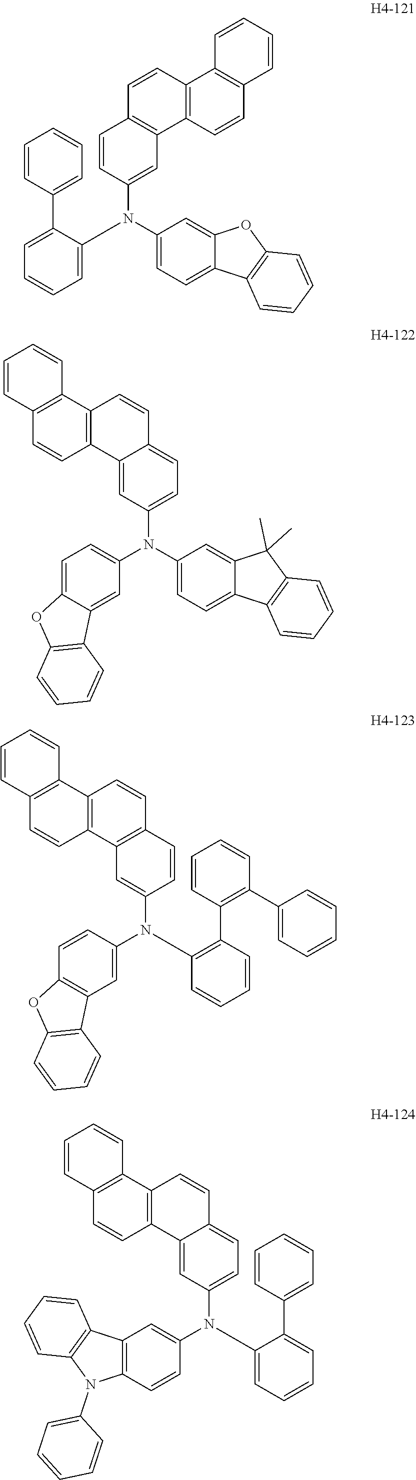

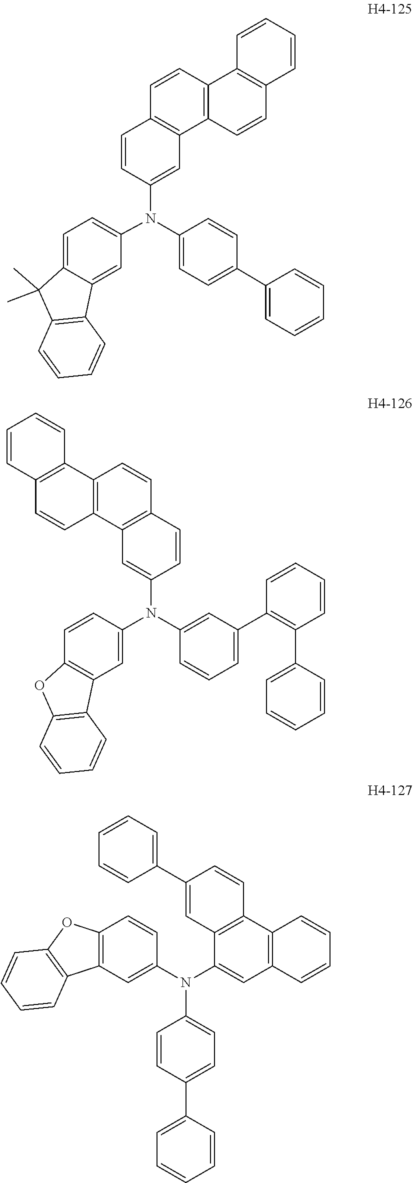

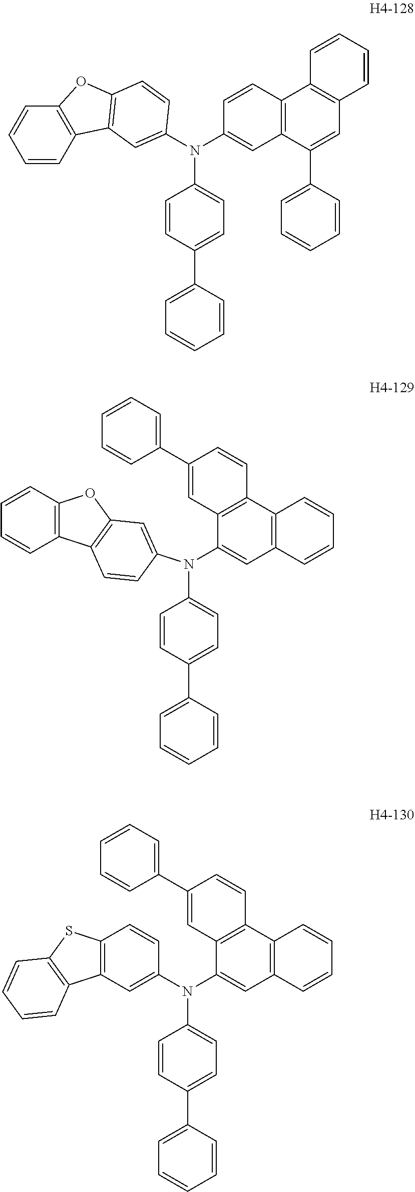 [SMILES: c1ccc(-c2ccc(N(c3ccc4c(c3)c(-c3ccccc3)cc3ccccc34)c3ccc4oc5ccccc5c4c3)cc2)cc1 ; c1ccc(-c2ccc(N(c3ccc4c(c3)oc3ccccc34)c3cc4ccccc4c4ccc(-c5ccccc5)cc34)cc2)cc1 ; c1ccc(-c2ccc(N(c3ccc4sc5ccccc5c4c3)c3cc4ccccc4c4ccc(-c5ccccc5)cc34)cc2)cc1]

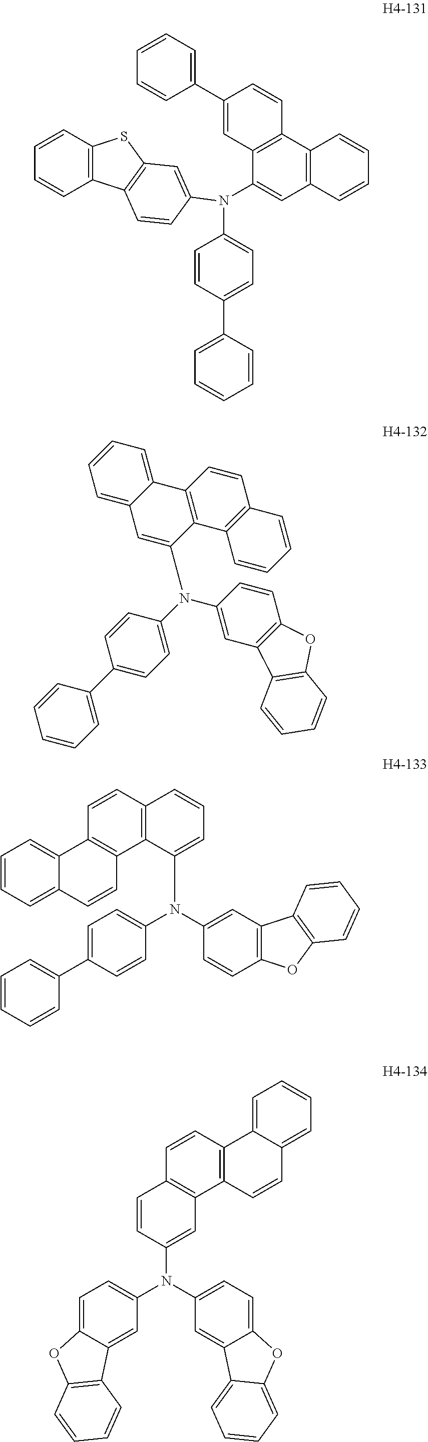 [SMILES: c1ccc(-c2ccc(N(c3ccc4c(c3)sc3ccccc34)c3cc4ccccc4c4ccc(-c5ccccc5)cc34)cc2)cc1 ; c1ccc(-c2ccc(N(c3ccc4oc5ccccc5c4c3)c3cc4ccccc4c4ccc5ccccc5c34)cc2)cc1 ; c1ccc(-c2ccc(N(c3ccc4oc5ccccc5c4c3)c3cccc4ccc5c6ccccc6ccc5c34)cc2)cc1 ; c1ccc2c(c1)ccc1c3cc(N(c4ccc5oc6ccccc6c5c4)c4ccc5oc6ccccc6c5c4)ccc3ccc21]

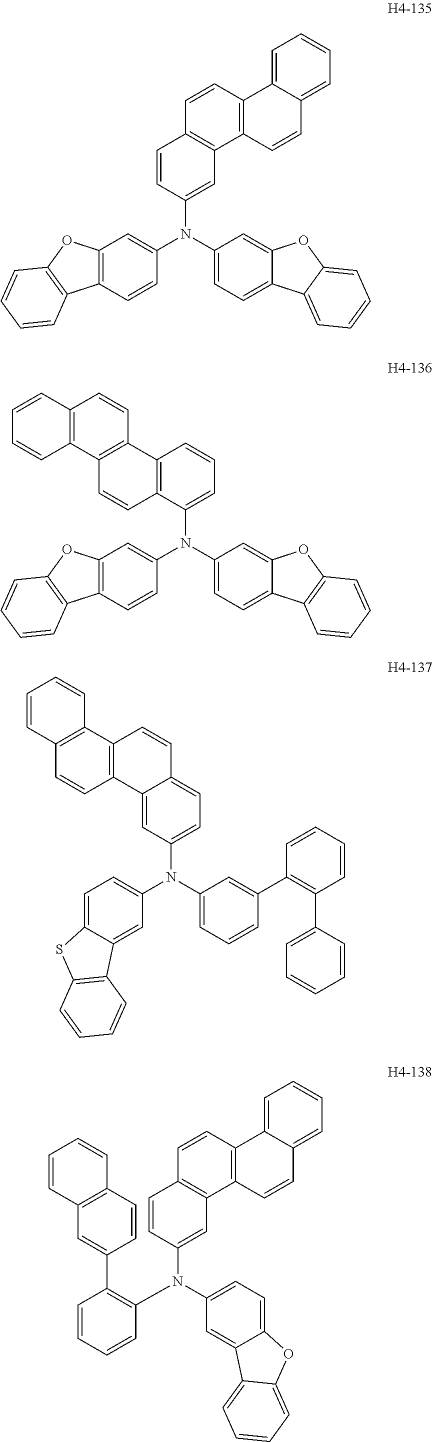

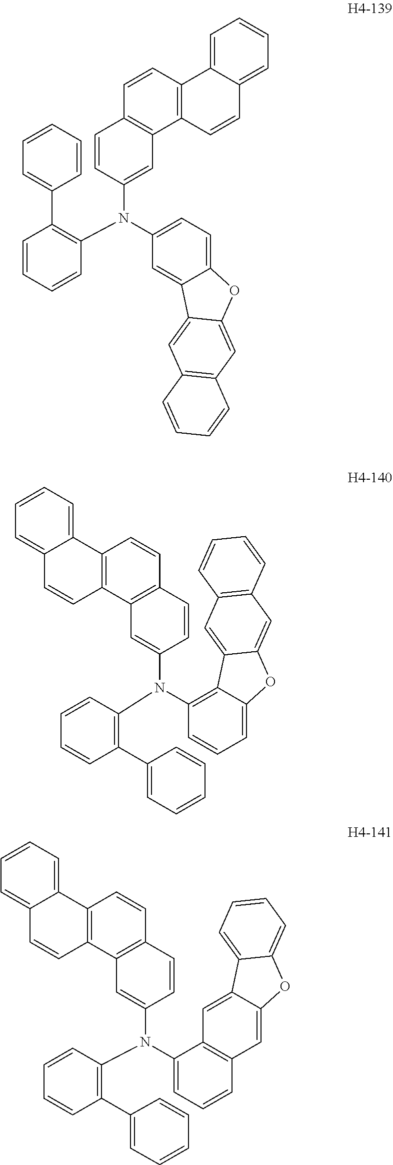 [SMILES: c1ccc(-c2ccccc2N(c2ccc3ccc4c5ccccc5ccc4c3c2)c2ccc3oc4cc5ccccc5cc4c3c2)cc1 ; c1ccc(-c2ccccc2N(c2ccc3ccc4c5ccccc5ccc4c3c2)c2cccc3oc4cc5ccccc5cc4c23)cc1 ; c1ccc(-c2ccccc2N(c2ccc3ccc4c5ccccc5ccc4c3c2)c2cccc3cc4oc5ccccc5c4cc23)cc1]

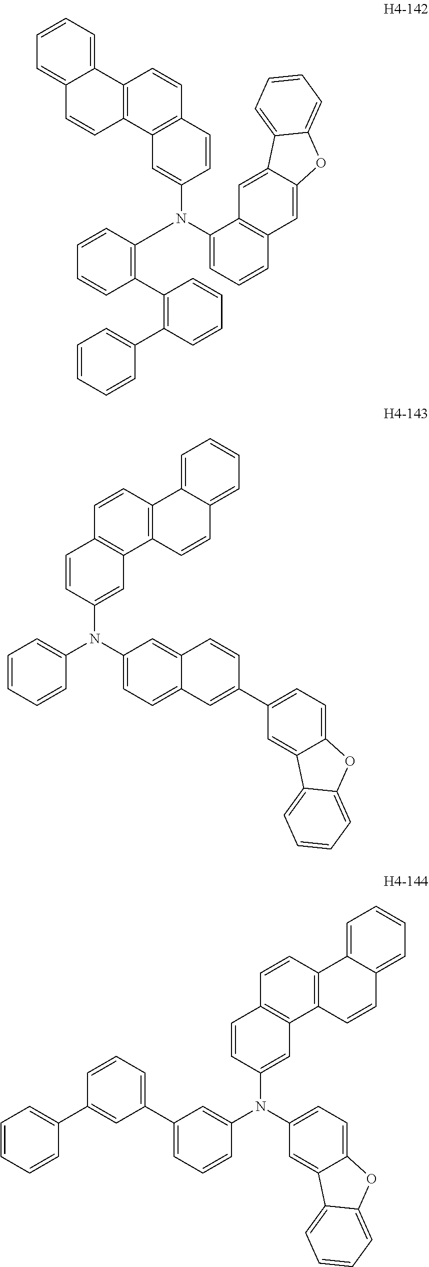 [SMILES: c1ccc(-c2ccccc2-c2ccccc2N(c2ccc3ccc4c5ccccc5ccc4c3c2)c2cccc3cc4oc5ccccc5c4cc23)cc1 ; c1ccc(N(c2ccc3cc(-c4ccc5oc6ccccc6c5c4)ccc3c2)c2ccc3ccc4c5ccccc5ccc4c3c2)cc1 ; c1ccc(-c2cccc(-c3cccc(N(c4ccc5ccc6c7ccccc7ccc6c5c4)c4ccc5oc6ccccc6c5c4)c3)c2)cc1]

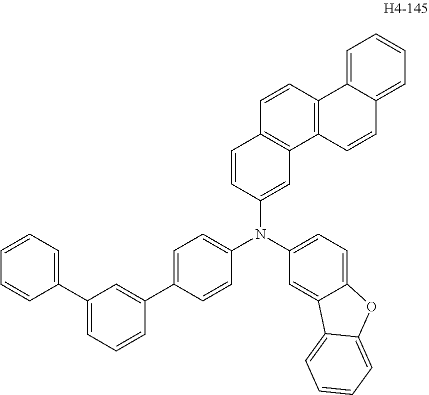 [SMILES: c1ccc(-c2cccc(-c3ccc(N(c4ccc5ccc6c7ccccc7ccc6c5c4)c4ccc5oc6ccccc6c5c4)cc3)c2)cc1]

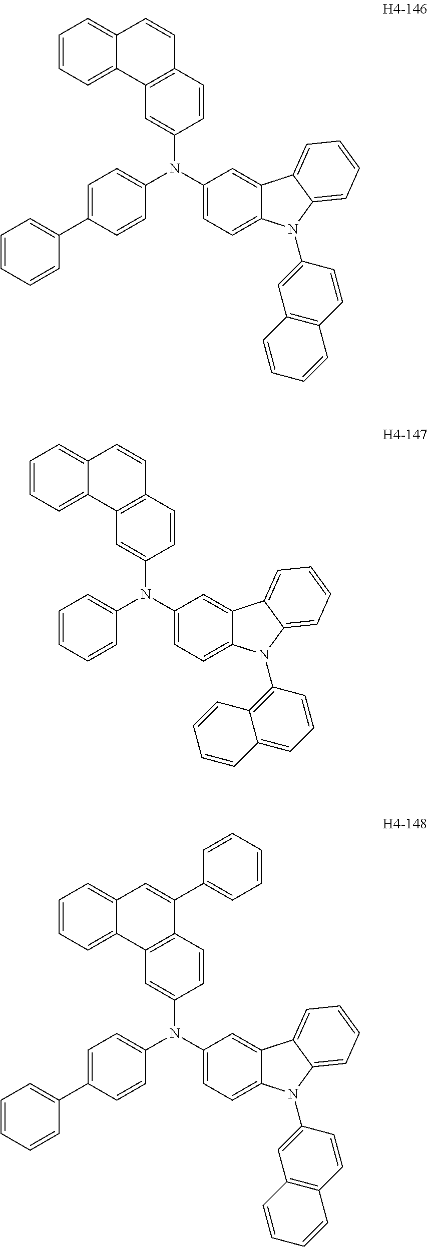 [SMILES: c1ccc(-c2ccc(N(c3ccc4ccc5ccccc5c4c3)c3ccc4c(c3)c3ccccc3n4-c3ccc4ccccc4c3)cc2)cc1 ; c1ccc(N(c2ccc3ccc4ccccc4c3c2)c2ccc3c(c2)c2ccccc2n3-c2cccc3ccccc23)cc1 ; c1ccc(-c2ccc(N(c3ccc4c(-c5ccccc5)cc5ccccc5c4c3)c3ccc4c(c3)c3ccccc3n4-c3ccc4ccccc4c3)cc2)cc1]

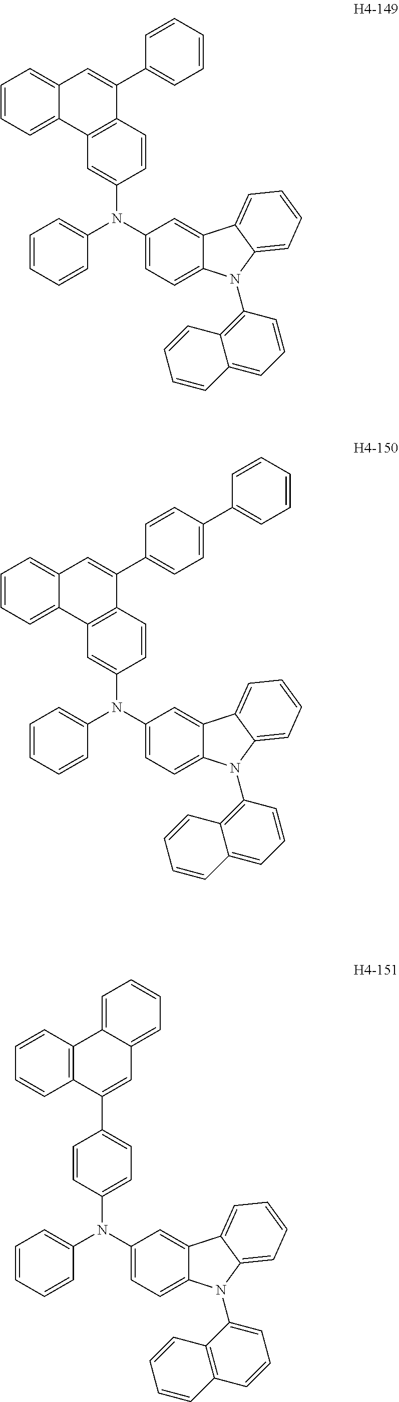 [SMILES: c1ccc(-c2cc3ccccc3c3cc(N(c4ccccc4)c4ccc5c(c4)c4ccccc4n5-c4cccc5ccccc45)ccc23)cc1 ; c1ccc(-c2ccc(-c3cc4ccccc4c4cc(N(c5ccccc5)c5ccc6c(c5)c5ccccc5n6-c5cccc6ccccc56)ccc34)cc2)cc1 ; c1ccc(N(c2ccc(-c3cc4ccccc4c4ccccc34)cc2)c2ccc3c(c2)c2ccccc2n3-c2cccc3ccccc23)cc1]

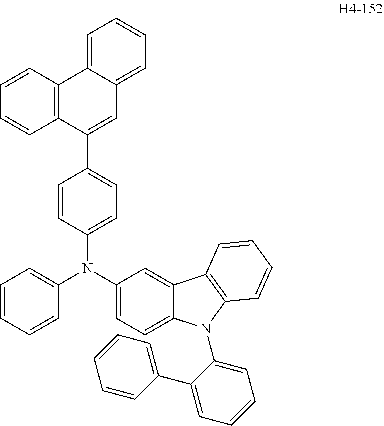 [SMILES: c1ccc(-c2ccccc2-n2c3ccccc3c3cc(N(c4ccccc4)c4ccc(-c5cc6ccccc6c6ccccc56)cc4)ccc32)cc1]

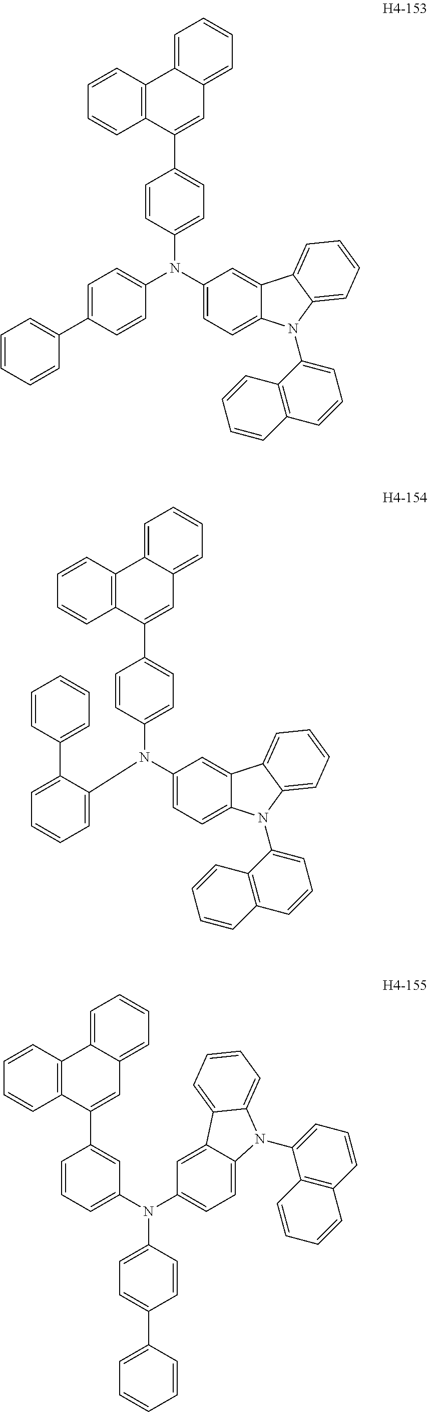 [SMILES: c1ccc(-c2ccc(N(c3ccc(-c4cc5ccccc5c5ccccc45)cc3)c3ccc4c(c3)c3ccccc3n4-c3cccc4ccccc34)cc2)cc1 ; c1ccc(-c2ccccc2N(c2ccc(-c3cc4ccccc4c4ccccc34)cc2)c2ccc3c(c2)c2ccccc2n3-c2cccc3ccccc23)cc1 ; c1ccc(-c2ccc(N(c3cccc(-c4cc5ccccc5c5ccccc45)c3)c3ccc4c(c3)c3ccccc3n4-c3cccc4ccccc34)cc2)cc1]

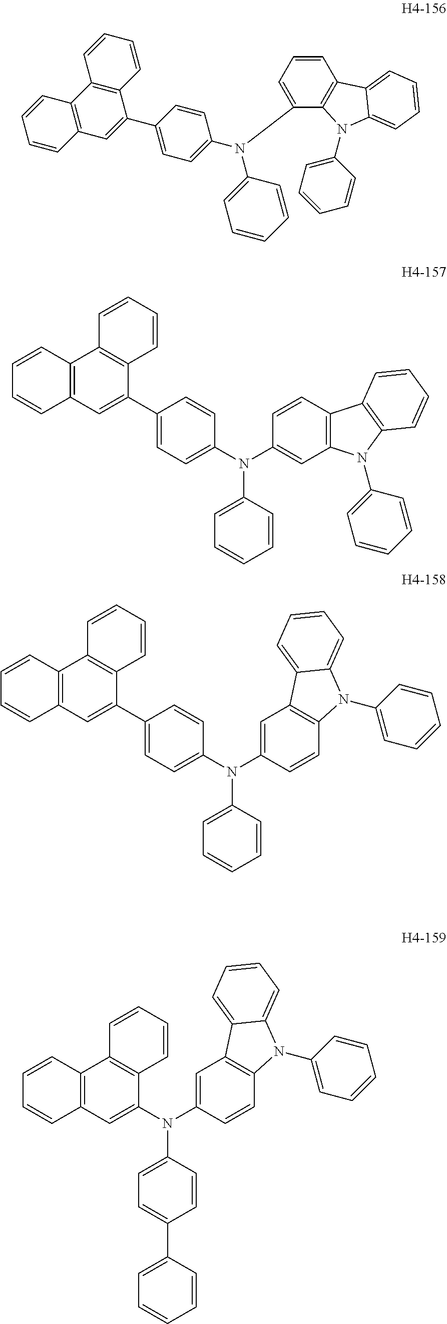 [SMILES: c1ccc(N(c2ccc(-c3cc4ccccc4c4ccccc34)cc2)c2cccc3c4ccccc4n(-c4ccccc4)c23)cc1 ; c1ccc(N(c2ccc(-c3cc4ccccc4c4ccccc34)cc2)c2ccc3c4ccccc4n(-c4ccccc4)c3c2)cc1 ; c1ccc(N(c2ccc(-c3cc4ccccc4c4ccccc34)cc2)c2ccc3c(c2)c2ccccc2n3-c2ccccc2)cc1 ; c1ccc(-c2ccc(N(c3ccc4c(c3)c3ccccc3n4-c3ccccc3)c3cc4ccccc4c4ccccc34)cc2)cc1]

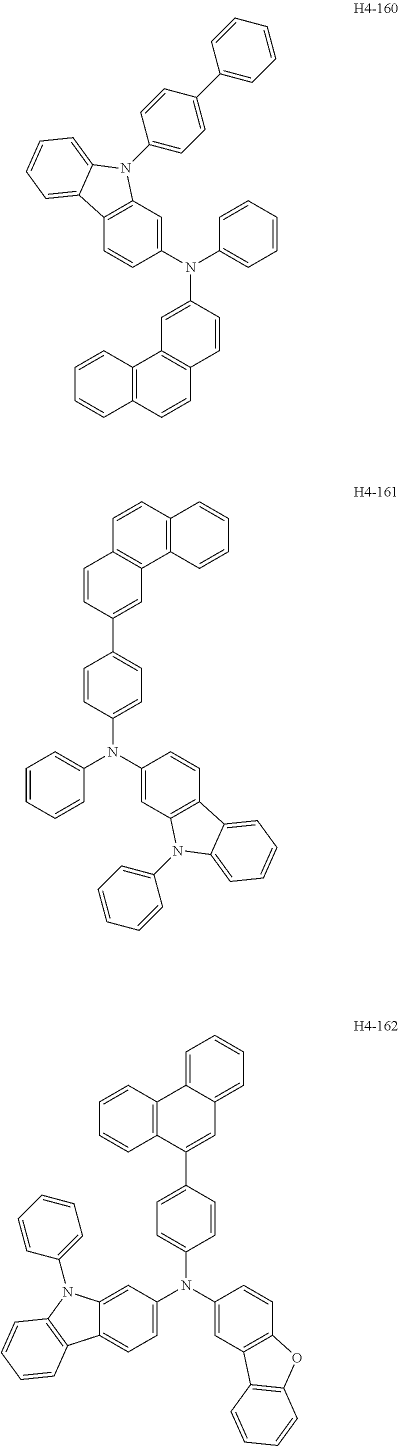 [SMILES: c1ccc(-c2ccc(-n3c4ccccc4c4ccc(N(c5ccccc5)c5ccc6ccc7ccccc7c6c5)cc43)cc2)cc1 ; c1ccc(N(c2ccc(-c3ccc4ccc5ccccc5c4c3)cc2)c2ccc3c4ccccc4n(-c4ccccc4)c3c2)cc1 ; c1ccc(-n2c3ccccc3c3ccc(N(c4ccc(-c5cc6ccccc6c6ccccc56)cc4)c4ccc5oc6ccccc6c5c4)cc32)cc1]

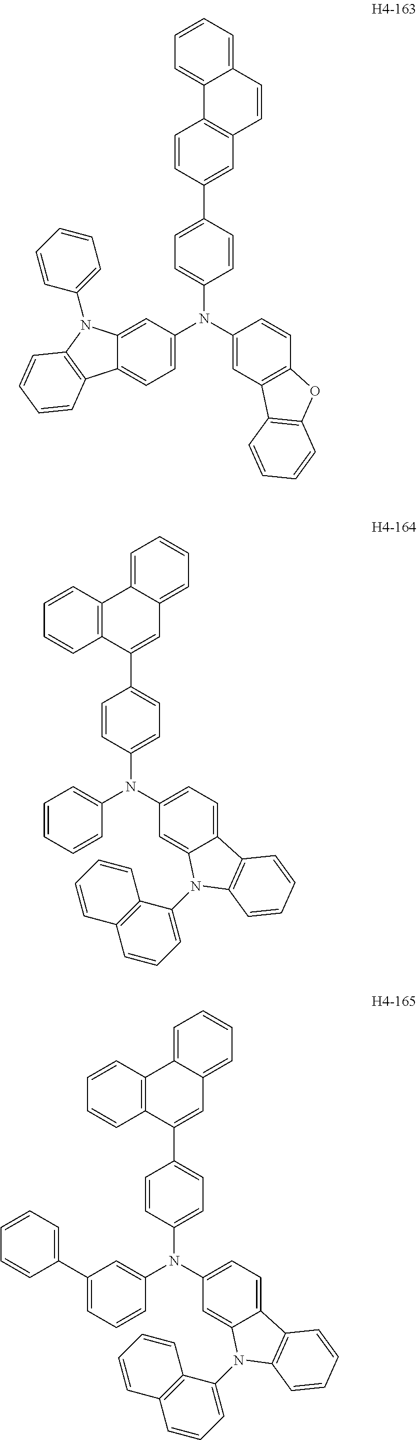 [SMILES: c1ccc(-n2c3ccccc3c3ccc(N(c4ccc(-c5ccc6c(ccc7ccccc76)c5)cc4)c4ccc5oc6ccccc6c5c4)cc32)cc1 ; c1ccc(N(c2ccc(-c3cc4ccccc4c4ccccc34)cc2)c2ccc3c4ccccc4n(-c4cccc5ccccc45)c3c2)cc1 ; c1ccc(-c2cccc(N(c3ccc(-c4cc5ccccc5c5ccccc45)cc3)c3ccc4c5ccccc5n(-c5cccc6ccccc56)c4c3)c2)cc1]

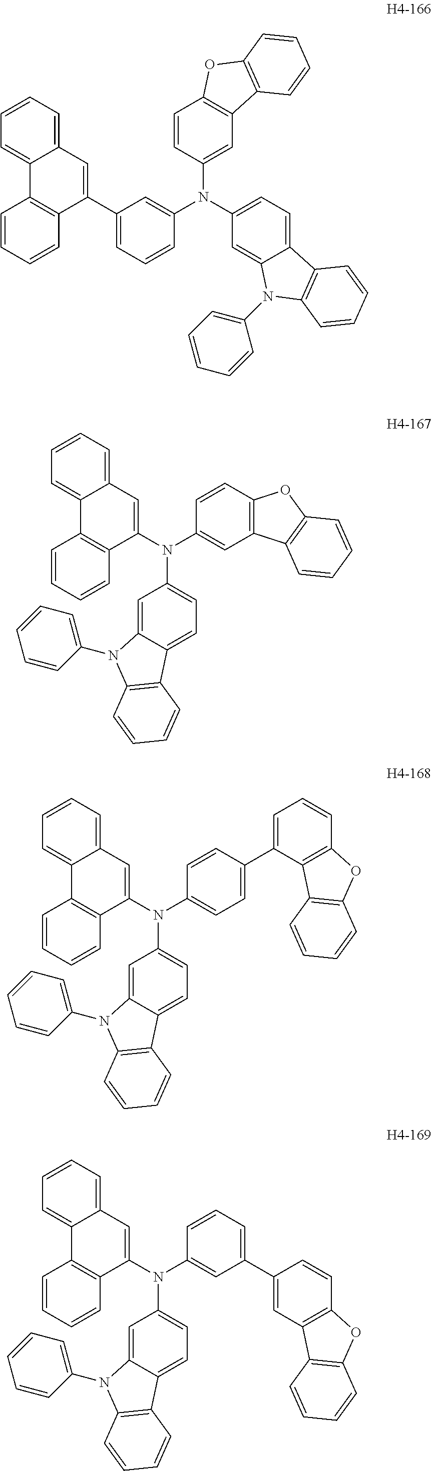 [SMILES: c1ccc(-n2c3ccccc3c3ccc(N(c4cccc(-c5cc6ccccc6c6ccccc56)c4)c4ccc5oc6ccccc6c5c4)cc32)cc1 ; c1ccc(-n2c3ccccc3c3ccc(N(c4ccc5oc6ccccc6c5c4)c4cc5ccccc5c5ccccc45)cc32)cc1 ; c1ccc(-n2c3ccccc3c3ccc(N(c4ccc(-c5cccc6oc7ccccc7c56)cc4)c4cc5ccccc5c5ccccc45)cc32)cc1 ; c1ccc(-n2c3ccccc3c3ccc(N(c4cccc(-c5ccc6oc7ccccc7c6c5)c4)c4cc5ccccc5c5ccccc45)cc32)cc1]

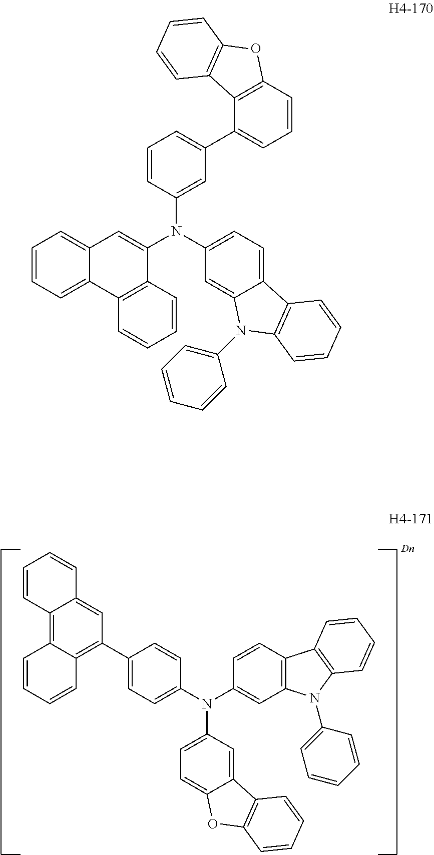

The compound represented by Formula 2 according to the present disclosure can also be prepared by way of a synthetic method known to those skilled in the art, and in particular, synthetic methods disclosed in many patent documents can be used. For example, the compound represented by Formula 2-1 according to the present disclosure can be prepared with reference to Korean Patent Application Laid-Open Nos. 2017-0022865 (published on Mar. 2, 2017) and 2018-0099487 (published on Sep. 5, 2018), but is not limited thereto. For example, the compound represented by Formula 2-2 can be prepared as shown in the following Reaction Scheme 2, but is not limited thereto. For example, the compound represented by Formula 2-3 can be prepared by way of a synthetic method known to those skilled in the art.

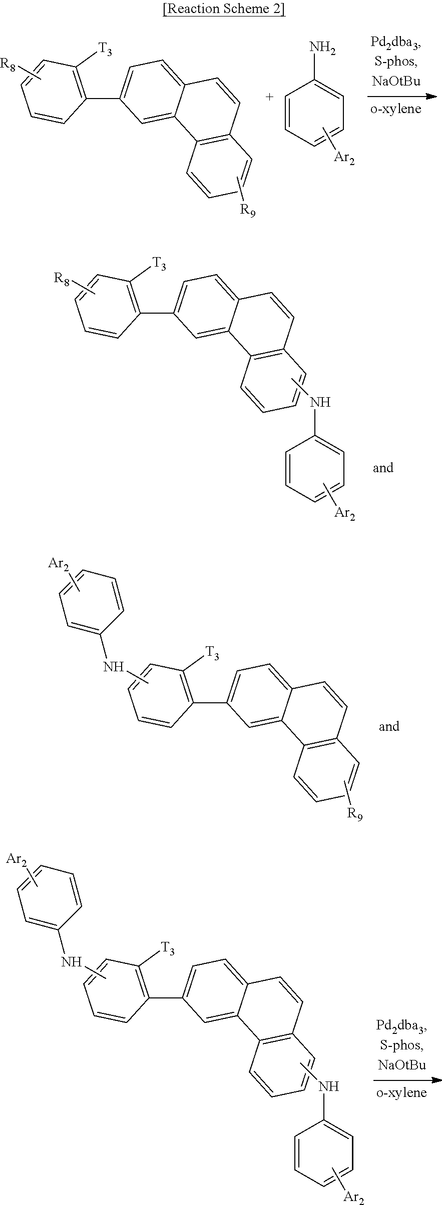

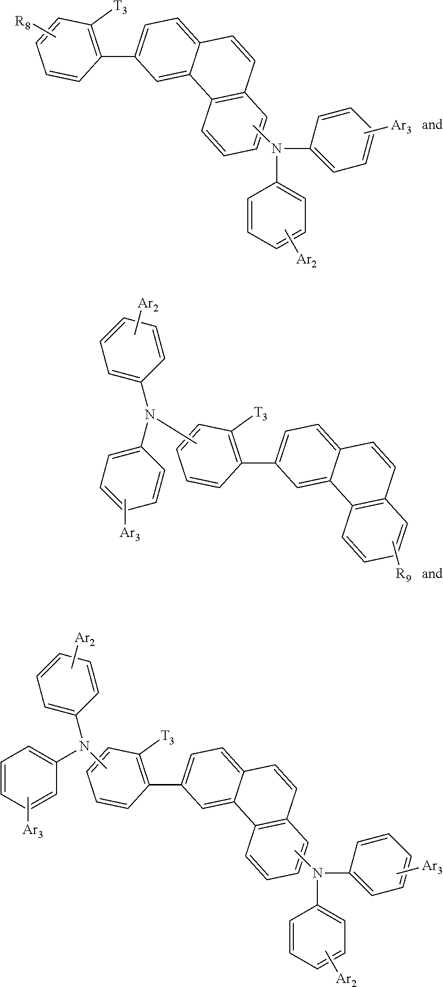

In Reaction Scheme 2, the definition of each substituent is as defined in Formula 2-2.

As described above, exemplary synthesis examples of the compound represented by Formula 2-2 according to the present disclosure are described, but these are based on the Suzuki cross-coupling reaction, Wittig reaction, Buchwald-Hartwig cross-coupling reaction, Miyaura borylation reaction, N-arylation reaction, H-mont-mediated etherification reaction, intramolecular acid-induced cyclization reaction, Pd(I1)-catalyzed oxidative cyclization reaction, Grignard reaction, Heck reaction, cyclic dehydration reaction, S_{N}¹ substitution reaction, S_{N2} substitution reaction, phosphine-mediated reductive cyclization reaction, etc. It will be understood by one skilled in the art that the above reaction proceeds even if other substituents defined in Formula 2-2 other than the substituents described in the specific synthesis examples are bonded.

The plurality of host materials according to one embodiment may further include a third host material comprising a compound represented by the following Formula 3.

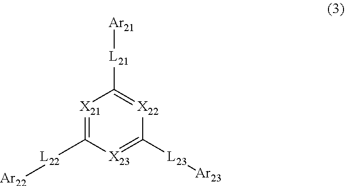

In Formula 3,
X₂₁ to X₂₃ each independently represent N or CR₂₀; provided that at least one of X₂₁ to X₂₃ is N;
R₂₀ represents hydrogen, deuterium, a halogen, a cyano, a substituted or unsubstituted (C₁-C₃₀)alkyl, a substituted or unsubstituted (C₆-C₃₀)aryl, a substituted or unsubstituted (3- to 30-membered)heteroaryl, a substituted or unsubstituted (C₃-C₃₀)cycloalkyl, a substituted or unsubstituted (C₁-C₃₀)alkoxy, a substituted or unsubstituted tri(C₁-C₃₀)alkylsilyl, a substituted or unsubstituted di(C₁-C₃₀)alkyl(C₆-C₃₀)arylsilyl, a substituted or unsubstituted (C₁-C₃₀)alkyldi(C₆-C₃₀)arylsilyl, a substituted or unsubstituted tri(C₆-C₃₀)arylsilyl, or a substituted or unsubstituted fused ring of (C₃-C₃₀) aliphatic ring and (C₆-C₃₀) aromatic ring;
L₂₁ to L₂₃ each independently represent a single bond, a substituted or unsubstituted (C₆-C₃₀)arylene, or a substituted or unsubstituted (3- to 30-membered)heteroarylene; and
Ar₂₁ to Ar₂₃ each independently represent a substituted or unsubstituted (C₁-C₃₀)alkyl, a substituted or unsubstituted (C₃-C₃₀)cycloalkyl, a substituted or unsubstituted (3- to 7-membered)heterocycloalkyl, a substituted or unsubstituted fused ring of (C₃-C₃₀) aliphatic ring and (C₆-C₃₀) aromatic ring, a substituted or unsubstituted (C₆-C₃₀)aryl, a substituted or unsubstituted (3- to 30-membered)heteroaryl, a substituted or unsubstituted tri(C₁-C₃₀)alkylsilyl, a substituted or unsubstituted di(C₁-C₃₀)alkyl(C₆-C₃₀)arylsilyl, a substituted or unsubstituted (C₁-C₃₀)alkyldi(C₆-C₃₀)arylsilyl, or a substituted or unsubstituted tri(C₆-C₃₀)arylsilyl.

In one embodiment, L₂₁ may be a single bond, or a substituted or unsubstituted (C₆-C₃₀)arylene, and preferably L₂₁ may be a single bond, or a substituted or unsubstituted (C₆-C₂₅)arylene. For example, L₂₁ may be a single bond, a substituted or unsubstituted phenylene, a substituted or unsubstituted phenylnaphthylene, a substituted or unsubstituted biphenylene, a substituted or unsubstituted naphthylene, or a substituted or unsubstituted naphthylphenylene.

In one embodiment, L₂₂ and L₂₃ each independently may be a single bond, or a substituted or unsubstituted (C₆-C₃₀)arylene, preferably L₂₂ and L₂₃ each independently may be a single bond, or a substituted or unsubstituted (C₆-C₂₅)arylene. For example, L₂₂ and L₂₃ each independently may be a single bond, a substituted or unsubstituted phenylene, a substituted or unsubstituted biphenylene, a substituted or unsubstituted naphthylene, or a substituted or unsubstituted phenanthrylene.

In one embodiment, Ar₂₁ may be a substituted or unsubstituted (3- to 30-membered)heteroaryl, and preferably Ar₂₁ may be a substituted or unsubstituted (3- to 25-membered)heteroaryl. For example, Ar₂₁ may be a substituted or unsubstituted dibenzofuranyl, or a substituted or unsubstituted dibenzothiophenyl.

In one embodiment, Ar₂₂ and Ar₂₃ each independently may be a substituted or unsubstituted (C₆-C₃₀)aryl, or a substituted or unsubstituted (5- to 30-membered)heteroaryl, and preferably Ar₂₂ and Ar₂₃ each independently may be a substituted or unsubstituted (C₆-C₂₅)aryl, or a substituted or unsubstituted (5- to 25-membered)heteroaryl. For example, Ar₂₂ and Ar₂₃ each independently may be a substituted or unsubstituted phenyl, phenyl unsubstituted or substituted with deuterium, a substituted or unsubstituted biphenyl, a substituted or unsubstituted o-terphenyl, a substituted or unsubstituted m-terphenyl, a substituted or unsubstituted p-terphenyl, a substituted or unsubstituted naphthyl, a substituted or unsubstituted naphthylphenyl, a substituted or unsubstituted phenanthryl, a substituted or unsubstituted anthracenyl, a substituted or unsubstituted chrysenyl, a substituted or unsubstituted triphenylenyl, a substituted or unsubstituted fluoranthenyl, a substituted or unsubstituted dimethylfluorenyl, a substituted or unsubstituted diphenylfluorenyl, a substituted or unsubstituted spirobifluorenyl, a substituted or unsubstituted carbazolyl, carbazolyl unsubstituted or substituted with phenyl, a substituted or unsubstituted dibenzofuranyl, dibenzofuranyl unsubstituted or substituted with deuterium, a substituted or unsubstituted dibenzothiophenyl, or a substituted or unsubstituted benzonaphthofuranyl.

According to one embodiment, the compound represented by Formula 3 may be represented by any one of the following Formulas 3-1 to 3-4.

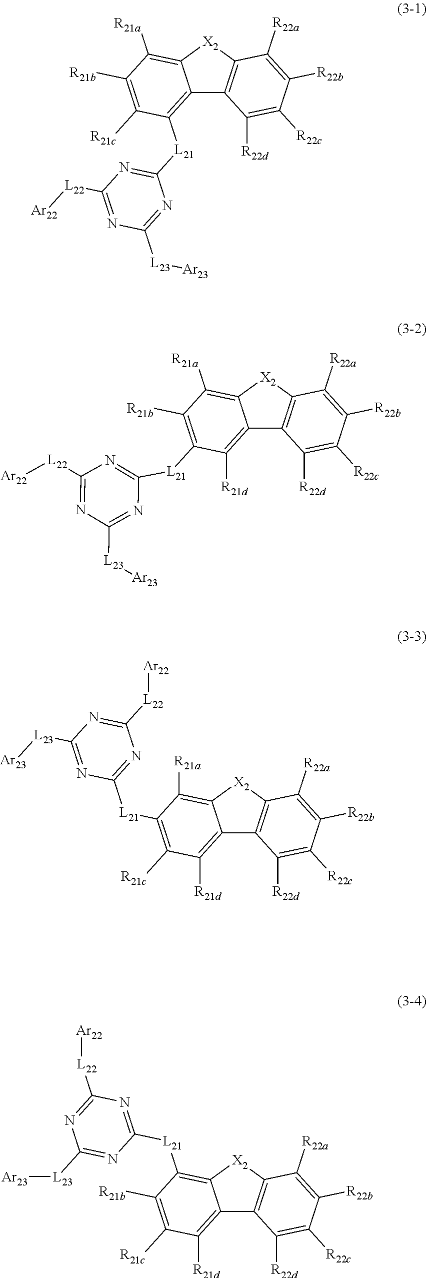

In Formulas 3-1 to 3-4,
X₂ represents O or S;
R₂₁ₐ to R_{21d} and R₂₂ₐ to R_{22d} each independently represent hydrogen, deuterium, a substituted or unsubstituted (C₆-C₃₀)aryl, or a substituted or unsubstituted (3- to 30-membered)heteroaryl; and
L₂₁ to L₂₃, Ar₂₂ and Ar₂₃ are as defined in Formula 3.

In one embodiment, R₂₁ₐ to R_{21d} and R₂₂ₐ to R_{22d} each independently may be hydrogen, deuterium, or a substituted or unsubstituted (C₆-C₃₀)aryl, preferably each independently may be hydrogen, deuterium, or a substituted or unsubstituted (C₆-C₂₅)aryl. For example, R₂₁ₐ to R_{21d} and R₂₂ₐ to R_{22d} each independently may be hydrogen, deuterium, a substituted or unsubstituted phenyl, a substituted or unsubstituted phenylnaphthyl, a substituted or unsubstituted biphenyl, a substituted or unsubstituted naphthyl, a substituted or unsubstituted naphthylphenyl, or a substituted or unsubstituted phenanthrenyl.

The compound represented by Formula 3 according to the present disclosure can be prepared by way of a synthetic method known to those skilled in the art, and in particular, synthetic methods disclosed in many patent documents can be used. For example, the compound represented by Formula 3 can be synthesized by referring to the methods disclosed in Korean Patent Application Laid-Open Nos. 2021-0124018 (published on Oct. 14, 2021) and 2021-0006283 (published on Jan. 18, 2021), but is not limited thereto.

According to one embodiment, the third host material may be more specifically illustrated by the following compounds, but is not limited thereto:

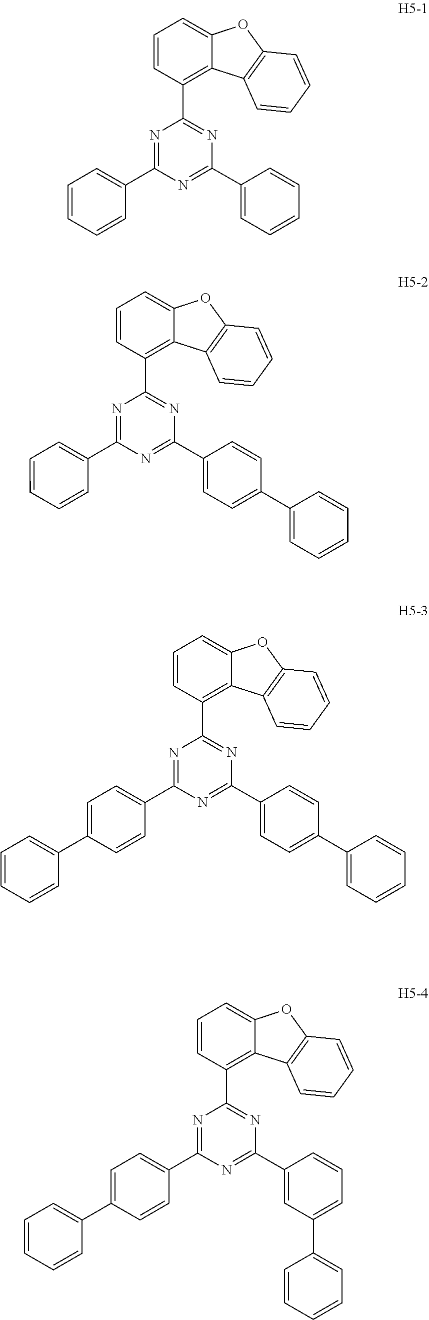 [SMILES: c1ccc(-c2nc(-c3ccccc3)nc(-c3cccc4oc5ccccc5c34)n2)cc1 ; c1ccc(-c2ccc(-c3nc(-c4ccccc4)nc(-c4cccc5oc6ccccc6c45)n3)cc2)cc1 ; c1ccc(-c2ccc(-c3nc(-c4ccc(-c5ccccc5)cc4)nc(-c4cccc5oc6ccccc6c45)n3)cc2)cc1 ; c1ccc(-c2ccc(-c3nc(-c4cccc(-c5ccccc5)c4)nc(-c4cccc5oc6ccccc6c45)n3)cc2)cc1]

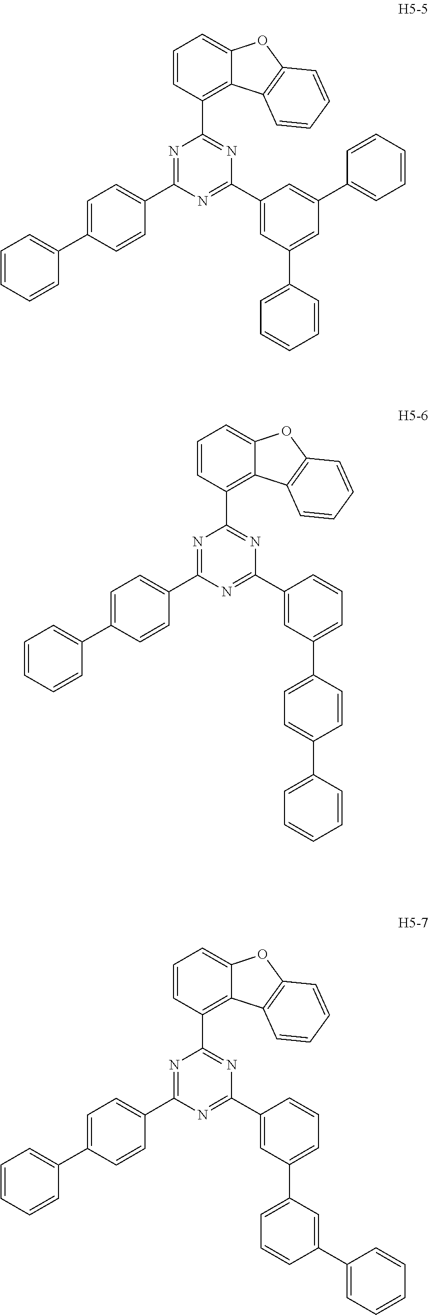 [SMILES: c1ccc(-c2ccc(-c3nc(-c4cc(-c5ccccc5)cc(-c5ccccc5)c4)nc(-c4cccc5oc6ccccc6c45)n3)cc2)cc1 ; c1ccc(-c2ccc(-c3cccc(-c4nc(-c5ccc(-c6ccccc6)cc5)nc(-c5cccc6oc7ccccc7c56)n4)c3)cc2)cc1 ; c1ccc(-c2ccc(-c3nc(-c4cccc(-c5cccc(-c6ccccc6)c5)c4)nc(-c4cccc5oc6ccccc6c45)n3)cc2)cc1]

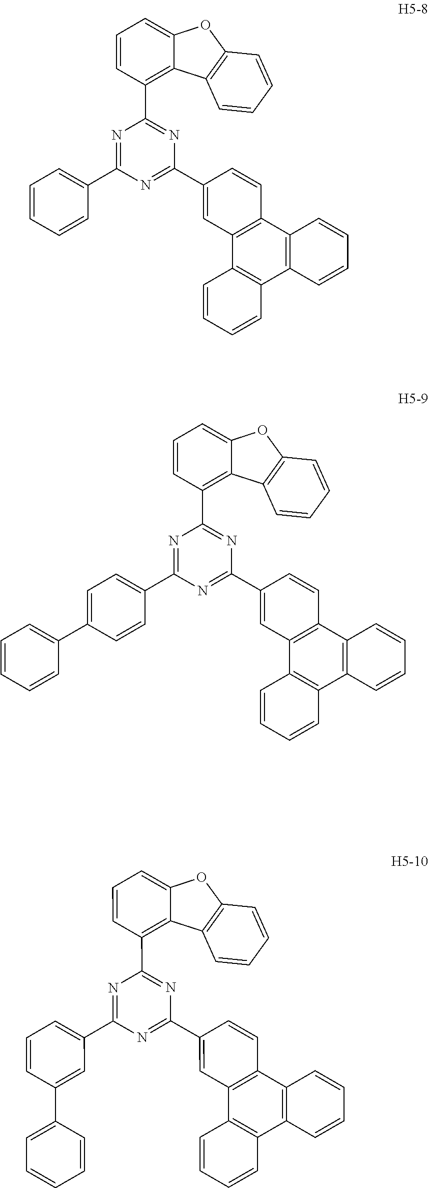 [SMILES: c1ccc(-c2nc(-c3ccc4c5ccccc5c5ccccc5c4c3)nc(-c3cccc4oc5ccccc5c34)n2)cc1 ; c1ccc(-c2ccc(-c3nc(-c4ccc5c6ccccc6c6ccccc6c5c4)nc(-c4cccc5oc6ccccc6c45)n3)cc2)cc1 ; c1ccc(-c2cccc(-c3nc(-c4ccc5c6ccccc6c6ccccc6c5c4)nc(-c4cccc5oc6ccccc6c45)n3)c2)cc1]

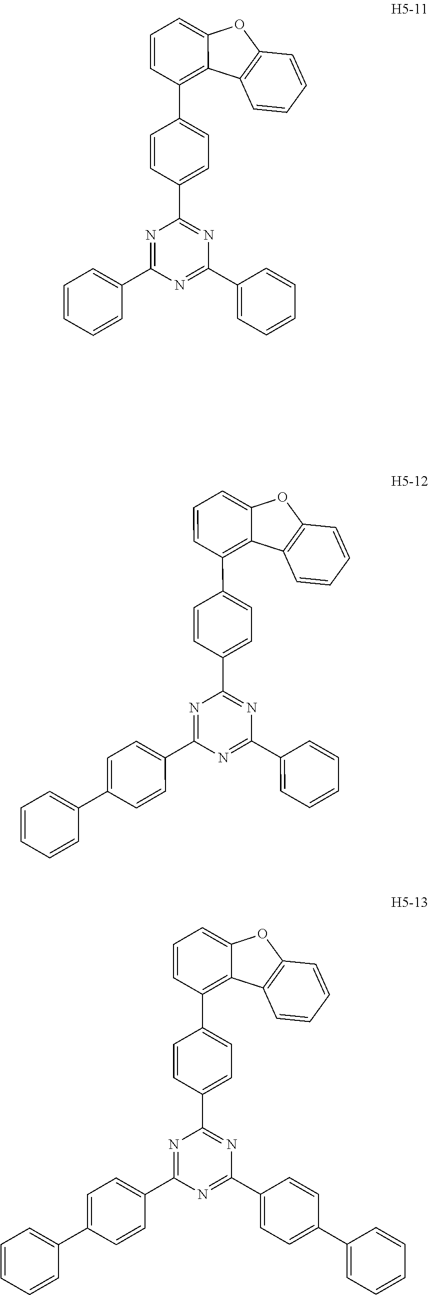 [SMILES: c1ccc(-c2nc(-c3ccccc3)nc(-c3ccc(-c4cccc5oc6ccccc6c45)cc3)n2)cc1 ; c1ccc(-c2ccc(-c3nc(-c4ccccc4)nc(-c4ccc(-c5cccc6oc7ccccc7c56)cc4)n3)cc2)cc1 ; c1ccc(-c2ccc(-c3nc(-c4ccc(-c5ccccc5)cc4)nc(-c4ccc(-c5cccc6oc7ccccc7c56)cc4)n3)cc2)cc1]

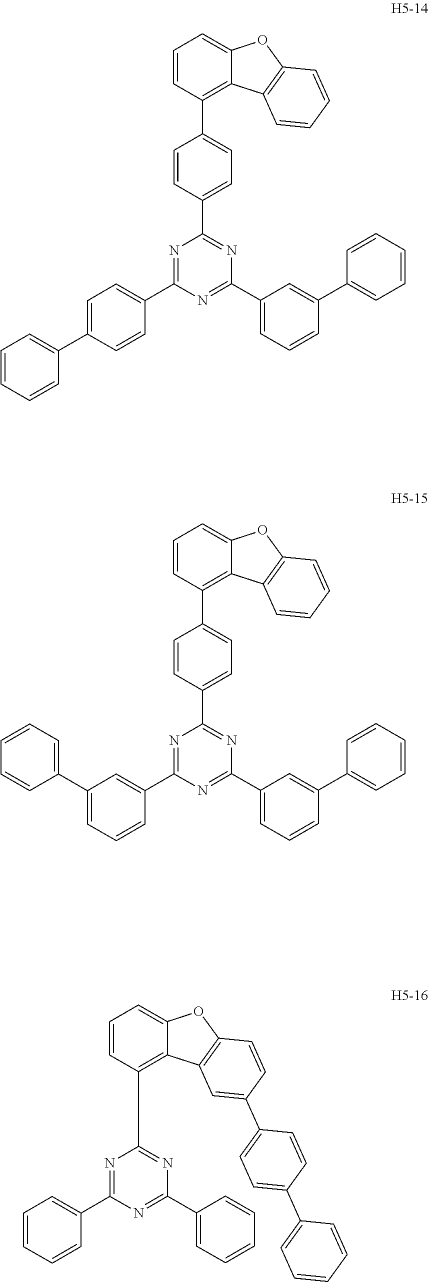 [SMILES: c1ccc(-c2ccc(-c3nc(-c4ccc(-c5cccc6oc7ccccc7c56)cc4)nc(-c4cccc(-c5ccccc5)c4)n3)cc2)cc1 ; c1ccc(-c2cccc(-c3nc(-c4ccc(-c5cccc6oc7ccccc7c56)cc4)nc(-c4cccc(-c5ccccc5)c4)n3)c2)cc1 ; c1ccc(-c2ccc(-c3ccc4oc5cccc(-c6nc(-c7ccccc7)nc(-c7ccccc7)n6)c5c4c3)cc2)cc1]

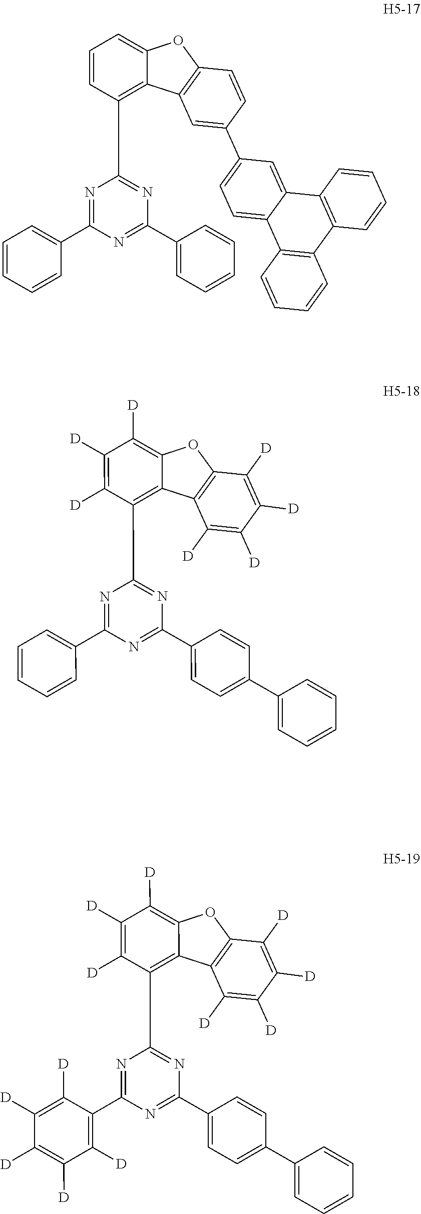 [SMILES: c1ccc(-c2nc(-c3ccccc3)nc(-c3cccc4oc5ccc(-c6ccc7c8ccccc8c8ccccc8c7c6)cc5c34)n2)cc1 ; [2H]c1c([2H])c([2H])c2c(oc3c([2H])c([2H])c([2H])c(-c4nc(-c5ccccc5)nc(-c5ccc(-c6ccccc6)cc5)n4)c32)c1[2H] ; [2H]c1c([2H])c([2H])c(-c2nc(-c3ccc(-c4ccccc4)cc3)nc(-c3c([2H])c([2H])c([2H])c4oc5c([2H])c([2H])c([2H])c([2H])c5c34)n2)c([2H])c1[2H]]

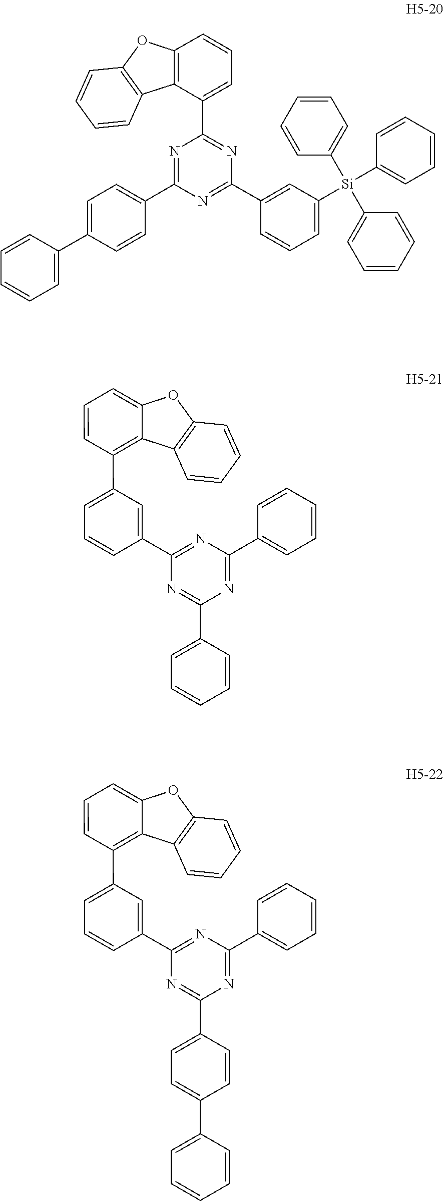 [SMILES: c1ccc(-c2ccc(-c3nc(-c4cccc([Si](c5ccccc5)(c5ccccc5)c5ccccc5)c4)nc(-c4cccc5oc6ccccc6c45)n3)cc2)cc1 ; c1ccc(-c2nc(-c3ccccc3)nc(-c3cccc(-c4cccc5oc6ccccc6c45)c3)n2)cc1 ; c1ccc(-c2ccc(-c3nc(-c4ccccc4)nc(-c4cccc(-c5cccc6oc7ccccc7c56)c4)n3)cc2)cc1]

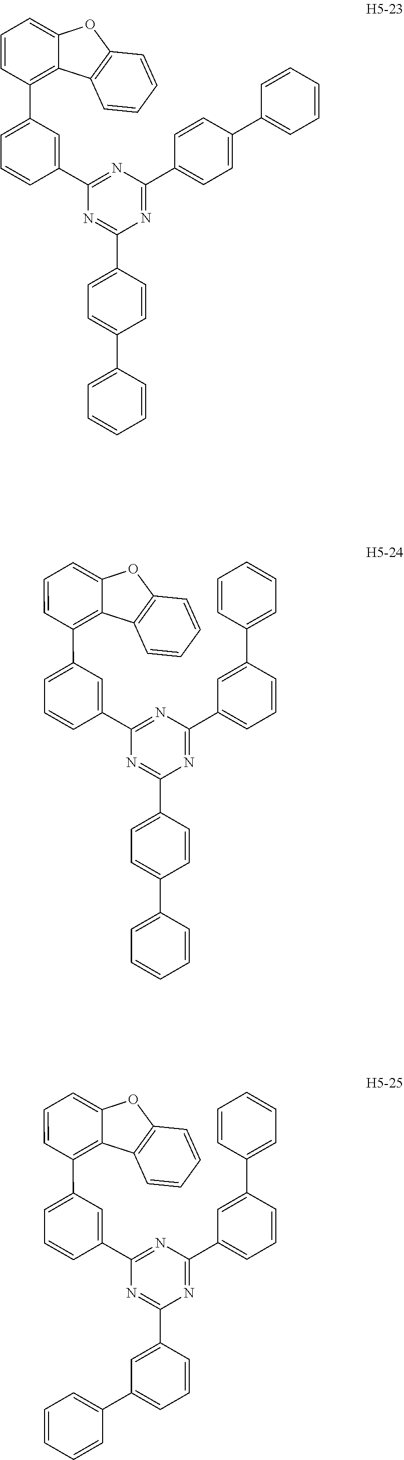 [SMILES: c1ccc(-c2ccc(-c3nc(-c4ccc(-c5ccccc5)cc4)nc(-c4cccc(-c5cccc6oc7ccccc7c56)c4)n3)cc2)cc1 ; c1ccc(-c2ccc(-c3nc(-c4cccc(-c5ccccc5)c4)nc(-c4cccc(-c5cccc6oc7ccccc7c56)c4)n3)cc2)cc1 ; c1ccc(-c2cccc(-c3nc(-c4cccc(-c5ccccc5)c4)nc(-c4cccc(-c5cccc6oc7ccccc7c56)c4)n3)c2)cc1]

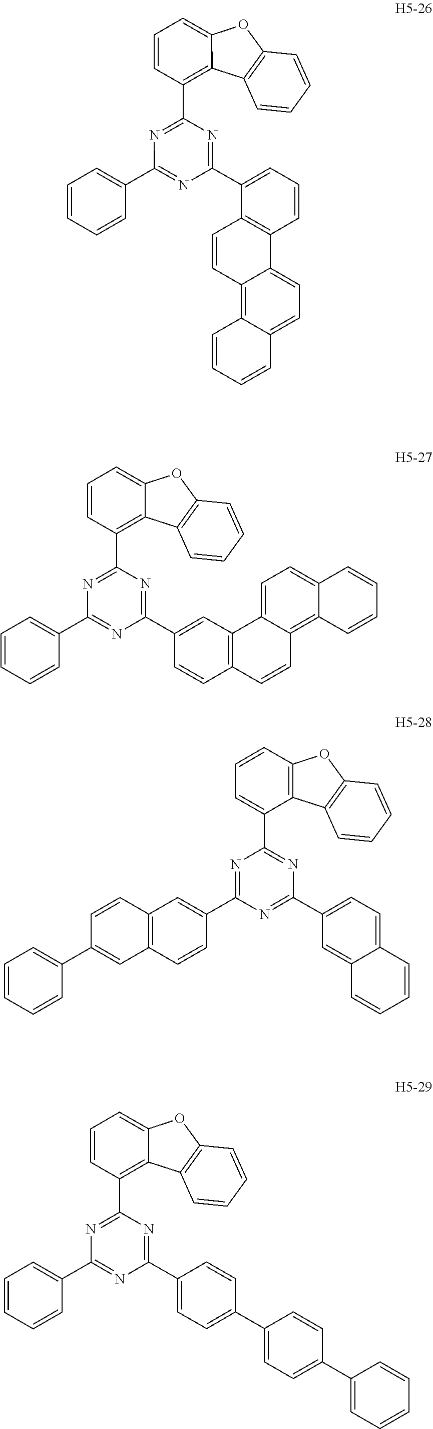 [SMILES: c1ccc(-c2nc(-c3cccc4c3ccc3c5ccccc5ccc43)nc(-c3cccc4oc5ccccc5c34)n2)cc1 ; c1ccc(-c2nc(-c3ccc4ccc5c6ccccc6ccc5c4c3)nc(-c3cccc4oc5ccccc5c34)n2)cc1 ; c1ccc(-c2ccc3cc(-c4nc(-c5ccc6ccccc6c5)nc(-c5cccc6oc7ccccc7c56)n4)ccc3c2)cc1 ; c1ccc(-c2ccc(-c3ccc(-c4nc(-c5ccccc5)nc(-c5cccc6oc7ccccc7c56)n4)cc3)cc2)cc1]

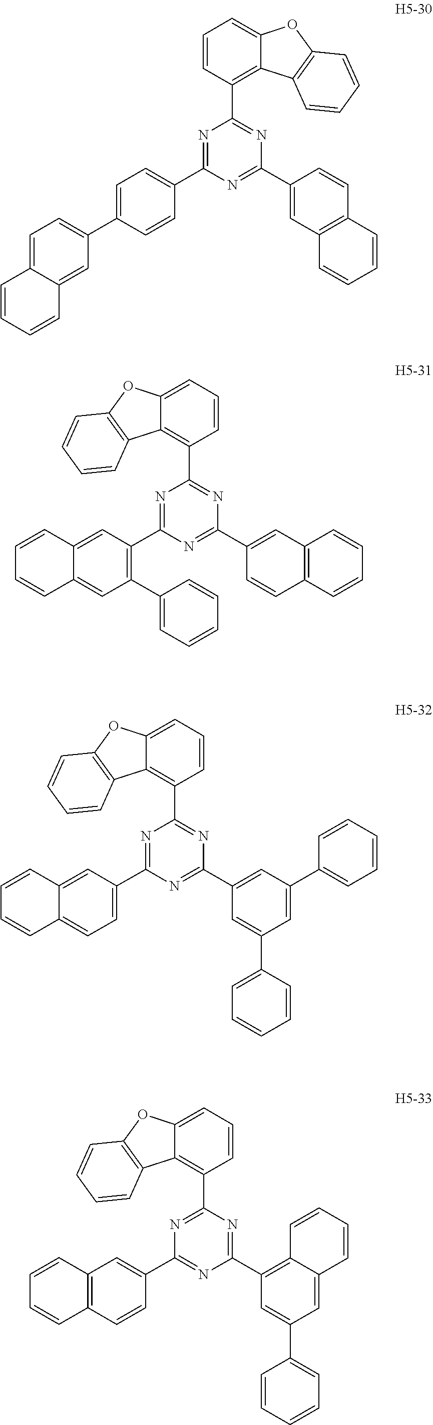 [SMILES: c1ccc2cc(-c3ccc(-c4nc(-c5ccc6ccccc6c5)nc(-c5cccc6oc7ccccc7c56)n4)cc3)ccc2c1 ; c1ccc(-c2cc3ccccc3cc2-c2nc(-c3ccc4ccccc4c3)nc(-c3cccc4oc5ccccc5c34)n2)cc1 ; c1ccc(-c2cc(-c3ccccc3)cc(-c3nc(-c4ccc5ccccc5c4)nc(-c4cccc5oc6ccccc6c45)n3)c2)cc1 ; c1ccc(-c2cc(-c3nc(-c4ccc5ccccc5c4)nc(-c4cccc5oc6ccccc6c45)n3)c3ccccc3c2)cc1]

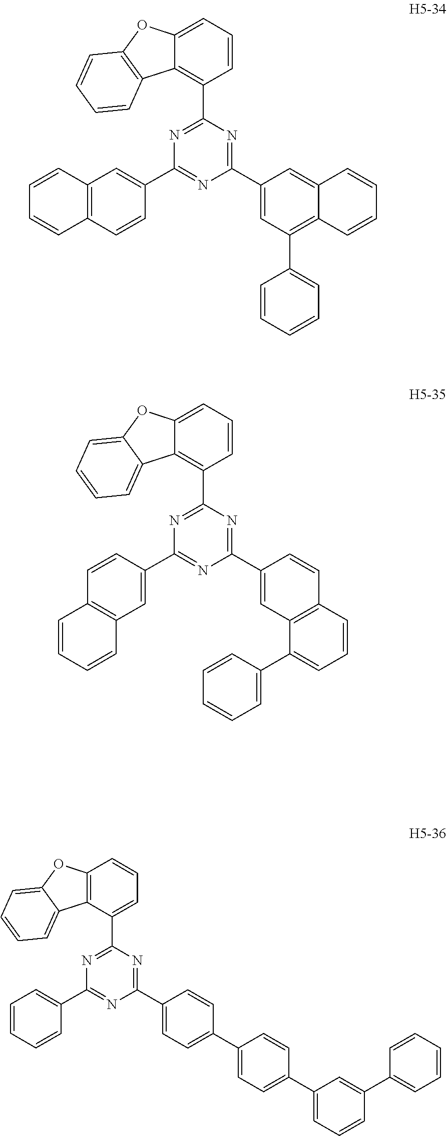 [SMILES: c1ccc(-c2cc(-c3nc(-c4ccc5ccccc5c4)nc(-c4cccc5oc6ccccc6c45)n3)cc3ccccc23)cc1 ; c1ccc(-c2cccc3ccc(-c4nc(-c5ccc6ccccc6c5)nc(-c5cccc6oc7ccccc7c56)n4)cc23)cc1 ; c1ccc(-c2cccc(-c3ccc(-c4ccc(-c5nc(-c6ccccc6)nc(-c6cccc7oc8ccccc8c67)n5)cc4)cc3)c2)cc1]

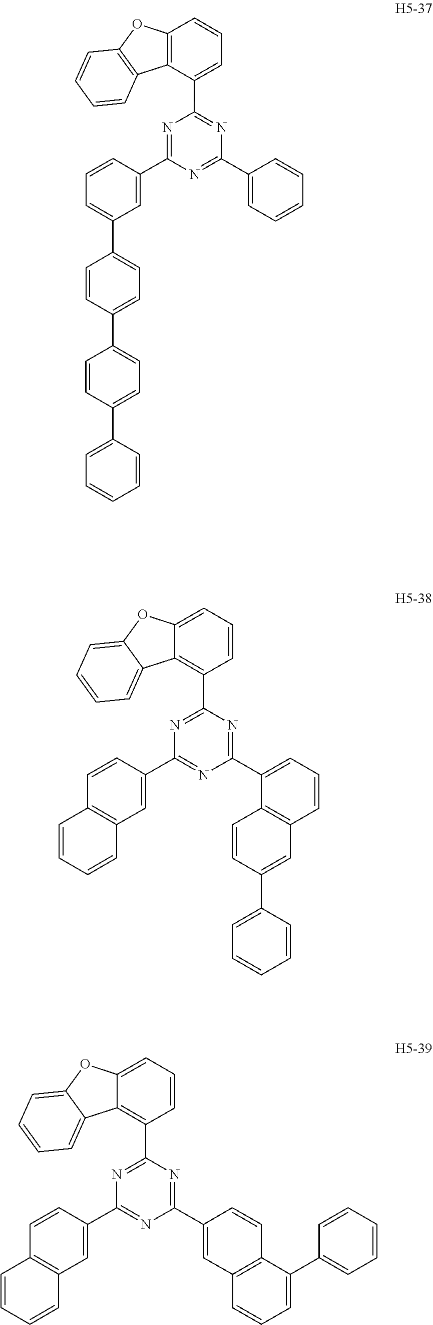 [SMILES: c1ccc(-c2ccc(-c3ccc(-c4cccc(-c5nc(-c6ccccc6)nc(-c6cccc7oc8ccccc8c67)n5)c4)cc3)cc2)cc1 ; c1ccc(-c2ccc3c(-c4nc(-c5ccc6ccccc6c5)nc(-c5cccc6oc7ccccc7c56)n4)cccc3c2)cc1 ; c1ccc(-c2cccc3cc(-c4nc(-c5ccc6ccccc6c5)nc(-c5cccc6oc7ccccc7c56)n4)ccc23)cc1]

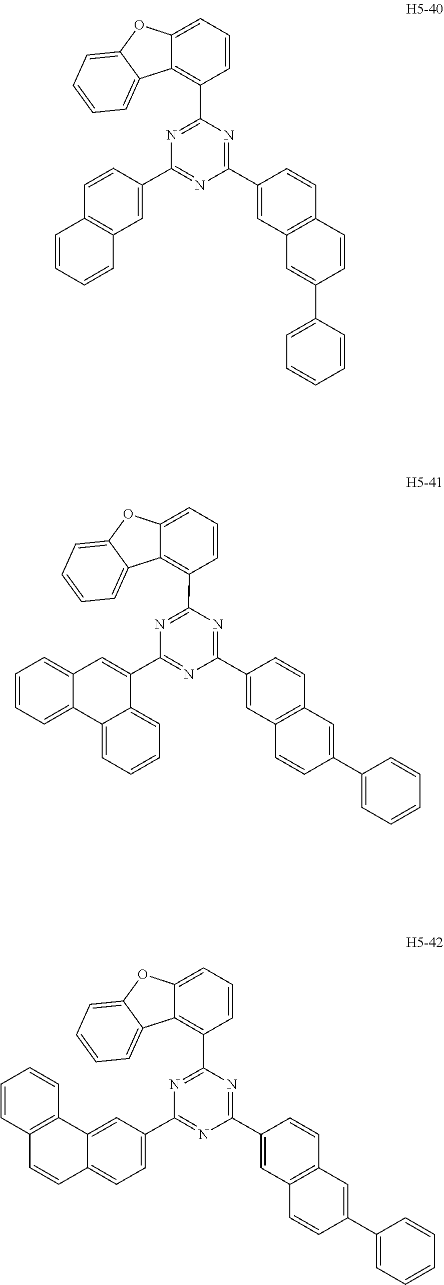 [SMILES: c1ccc(-c2ccc3ccc(-c4nc(-c5ccc6ccccc6c5)nc(-c5cccc6oc7ccccc7c56)n4)cc3c2)cc1 ; c1ccc(-c2ccc3cc(-c4nc(-c5cc6ccccc6c6ccccc56)nc(-c5cccc6oc7ccccc7c56)n4)ccc3c2)cc1 ; c1ccc(-c2ccc3cc(-c4nc(-c5ccc6ccc7ccccc7c6c5)nc(-c5cccc6oc7ccccc7c56)n4)ccc3c2)cc1]

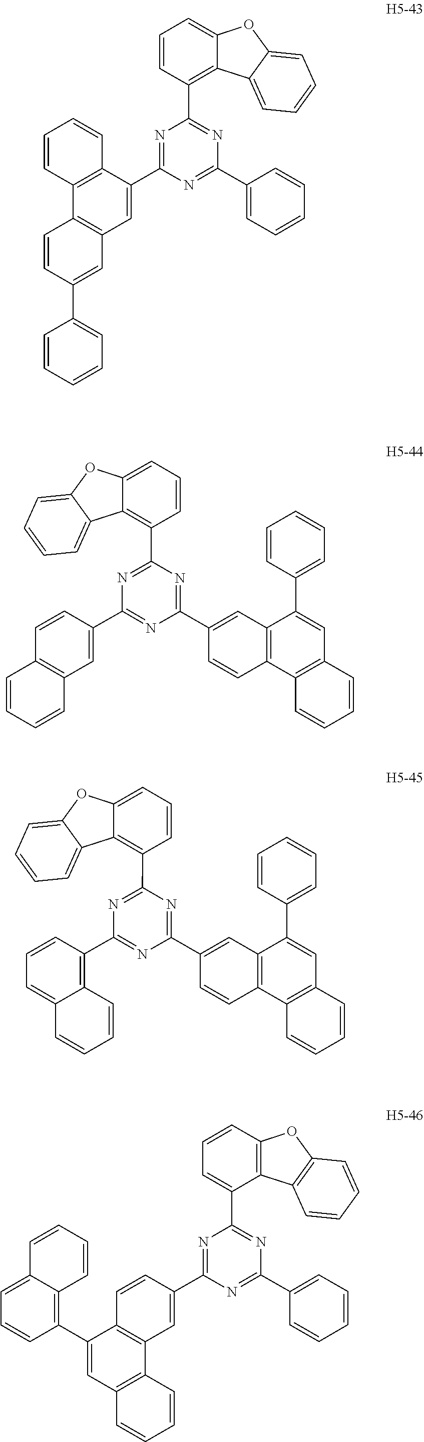 [SMILES: c1ccc(-c2ccc3c(c2)cc(-c2nc(-c4ccccc4)nc(-c4cccc5oc6ccccc6c45)n2)c2ccccc23)cc1 ; c1ccc(-c2cc3ccccc3c3ccc(-c4nc(-c5ccc6ccccc6c5)nc(-c5cccc6oc7ccccc7c56)n4)cc23)cc1 ; c1ccc(-c2cc3ccccc3c3ccc(-c4nc(-c5cccc6ccccc56)nc(-c5cccc6oc7ccccc7c56)n4)cc23)cc1 ; c1ccc(-c2nc(-c3ccc4c(-c5cccc6ccccc56)cc5ccccc5c4c3)nc(-c3cccc4oc5ccccc5c34)n2)cc1]

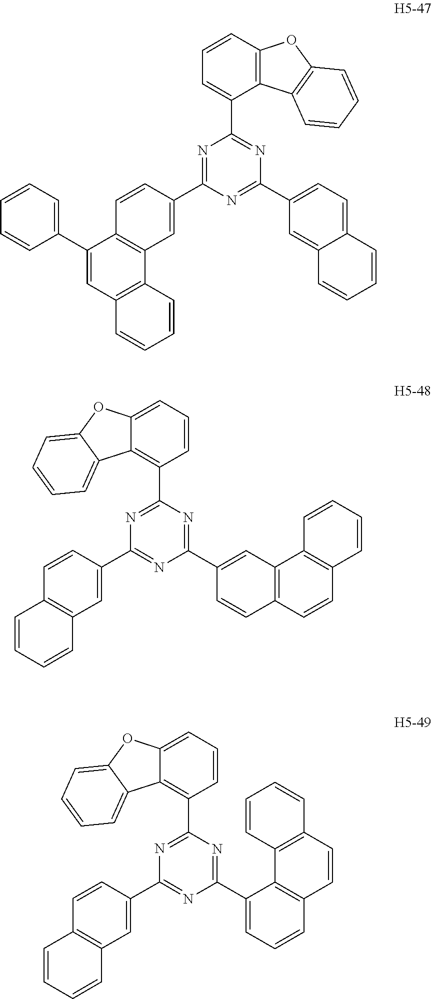 [SMILES: c1ccc(-c2cc3ccccc3c3cc(-c4nc(-c5ccc6ccccc6c5)nc(-c5cccc6oc7ccccc7c56)n4)ccc23)cc1 ; c1ccc2cc(-c3nc(-c4ccc5ccc6ccccc6c5c4)nc(-c4cccc5oc6ccccc6c45)n3)ccc2c1 ; c1ccc2cc(-c3nc(-c4cccc5ccc6ccccc6c45)nc(-c4cccc5oc6ccccc6c45)n3)ccc2c1]

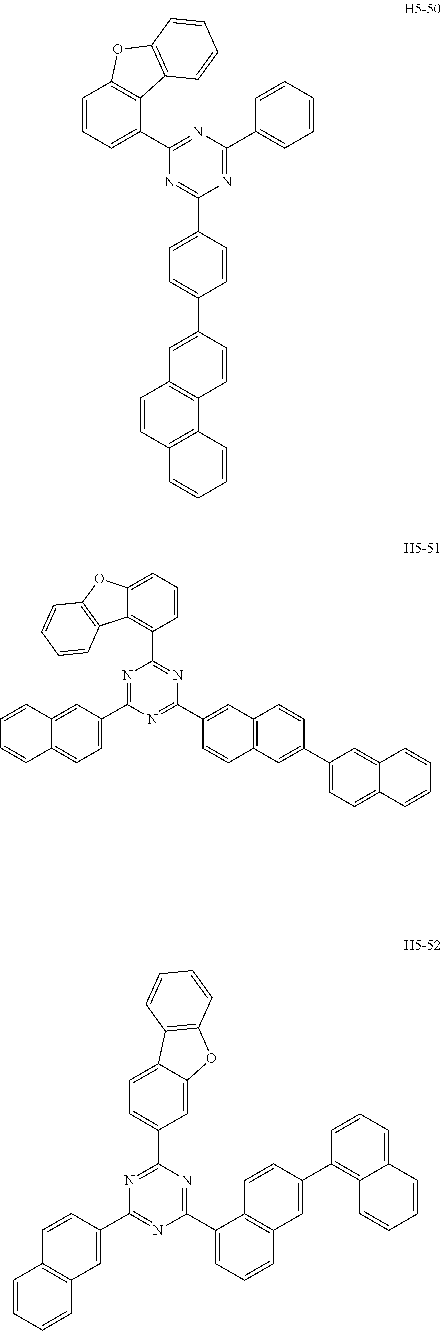 [SMILES: c1ccc(-c2nc(-c3ccc(-c4ccc5c(ccc6ccccc65)c4)cc3)nc(-c3cccc4oc5ccccc5c34)n2)cc1 ; c1ccc2cc(-c3ccc4cc(-c5nc(-c6ccc7ccccc7c6)nc(-c6cccc7oc8ccccc8c67)n5)ccc4c3)ccc2c1 ; c1ccc2cc(-c3nc(-c4ccc5c(c4)oc4ccccc45)nc(-c4cccc5cc(-c6cccc7ccccc67)ccc45)n3)ccc2c1]

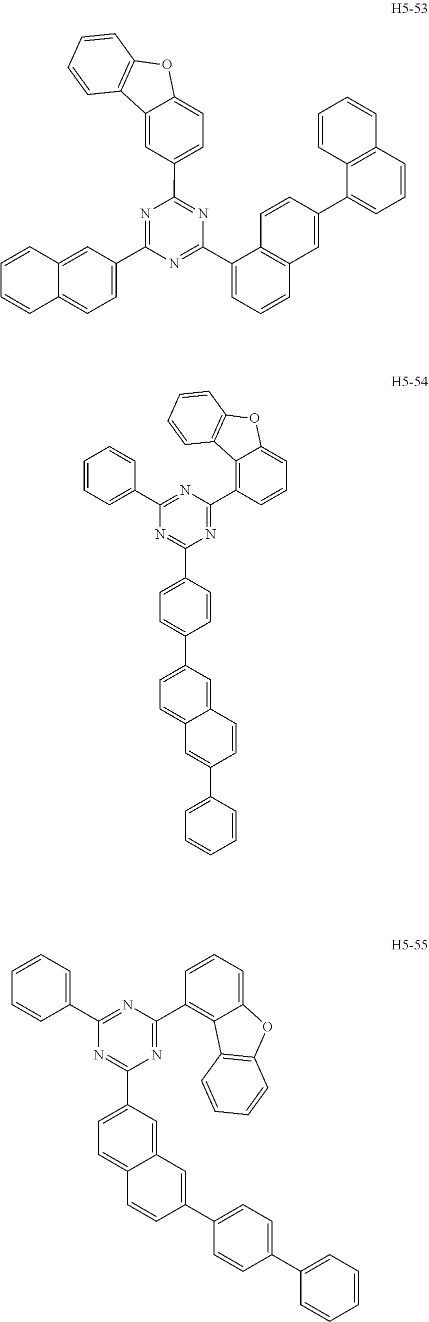 [SMILES: c1ccc2cc(-c3nc(-c4ccc5oc6ccccc6c5c4)nc(-c4cccc5cc(-c6cccc7ccccc67)ccc45)n3)ccc2c1 ; c1ccc(-c2ccc3cc(-c4ccc(-c5nc(-c6ccccc6)nc(-c6cccc7oc8ccccc8c67)n5)cc4)ccc3c2)cc1 ; c1ccc(-c2ccc(-c3ccc4ccc(-c5nc(-c6ccccc6)nc(-c6cccc7oc8ccccc8c67)n5)cc4c3)cc2)cc1]

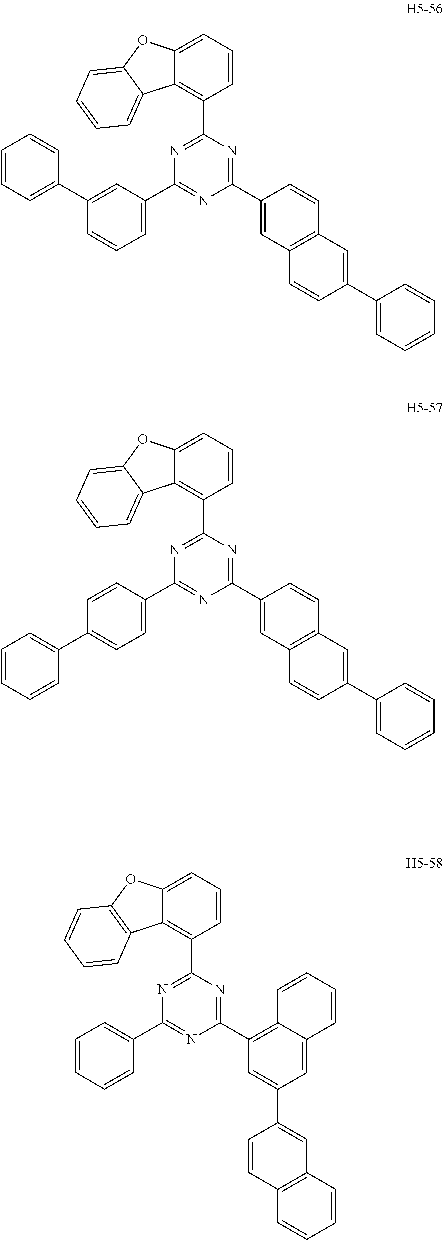 [SMILES: c1ccc(-c2cccc(-c3nc(-c4ccc5cc(-c6ccccc6)ccc5c4)nc(-c4cccc5oc6ccccc6c45)n3)c2)cc1 ; c1ccc(-c2ccc(-c3nc(-c4ccc5cc(-c6ccccc6)ccc5c4)nc(-c4cccc5oc6ccccc6c45)n3)cc2)cc1 ; c1ccc(-c2nc(-c3cc(-c4ccc5ccccc5c4)cc4ccccc34)nc(-c3cccc4oc5ccccc5c34)n2)cc1]

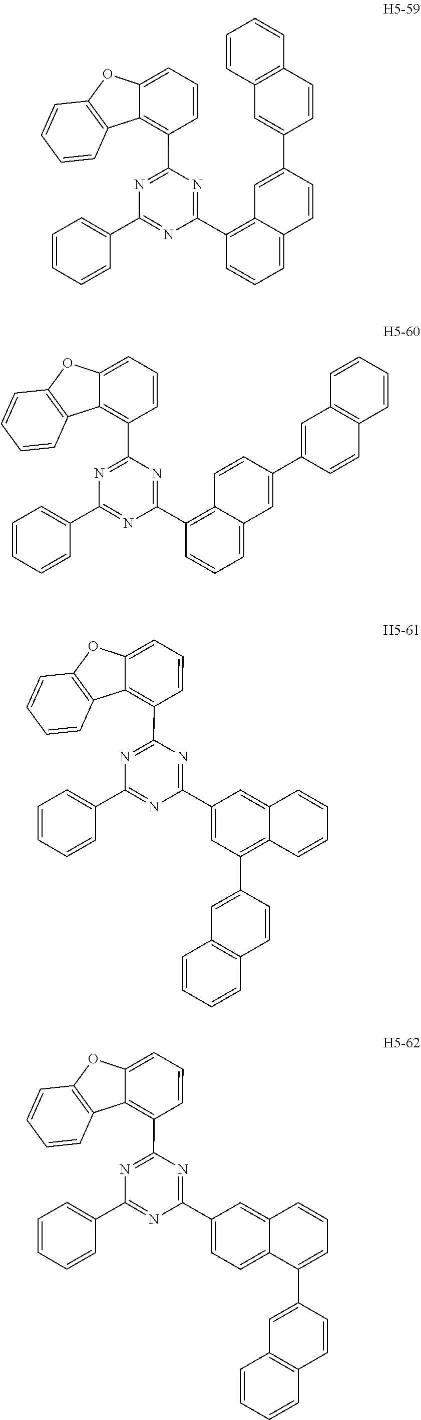 [SMILES: c1ccc(-c2nc(-c3cccc4ccc(-c5ccc6ccccc6c5)cc34)nc(-c3cccc4oc5ccccc5c34)n2)cc1 ; c1ccc(-c2nc(-c3cccc4cc(-c5ccc6ccccc6c5)ccc34)nc(-c3cccc4oc5ccccc5c34)n2)cc1 ; c1ccc(-c2nc(-c3cc(-c4ccc5ccccc5c4)c4ccccc4c3)nc(-c3cccc4oc5ccccc5c34)n2)cc1 ; c1ccc(-c2nc(-c3ccc4c(-c5ccc6ccccc6c5)cccc4c3)nc(-c3cccc4oc5ccccc5c34)n2)cc1]

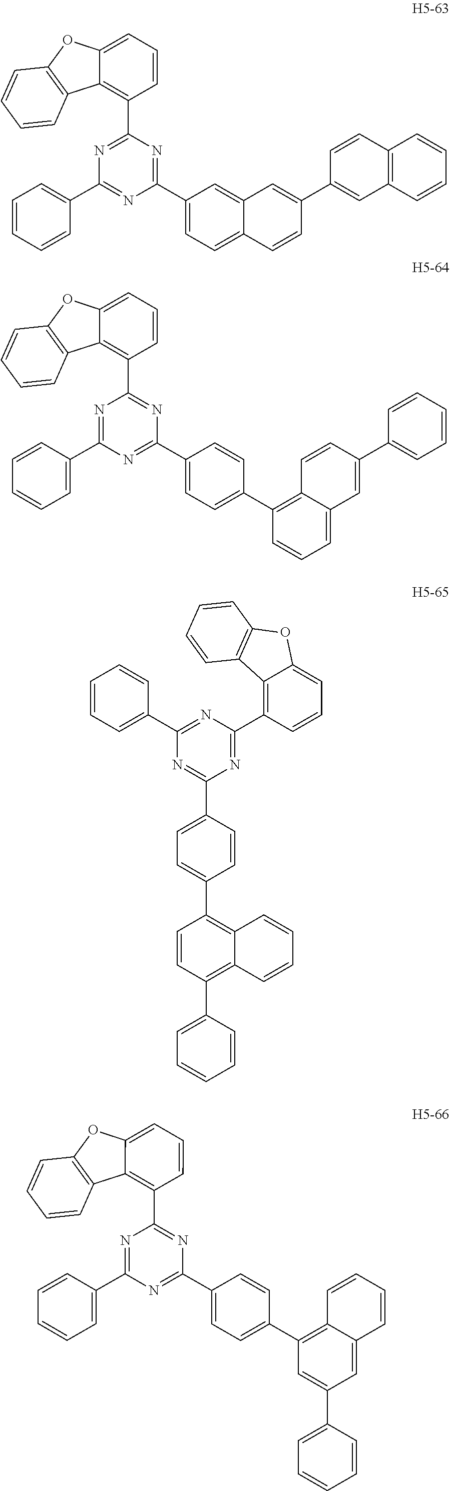 [SMILES: c1ccc(-c2nc(-c3ccc4ccc(-c5ccc6ccccc6c5)cc4c3)nc(-c3cccc4oc5ccccc5c34)n2)cc1 ; c1ccc(-c2ccc3c(-c4ccc(-c5nc(-c6ccccc6)nc(-c6cccc7oc8ccccc8c67)n5)cc4)cccc3c2)cc1 ; c1ccc(-c2nc(-c3ccc(-c4ccc(-c5ccccc5)c5ccccc45)cc3)nc(-c3cccc4oc5ccccc5c34)n2)cc1 ; c1ccc(-c2cc(-c3ccc(-c4nc(-c5ccccc5)nc(-c5cccc6oc7ccccc7c56)n4)cc3)c3ccccc3c2)cc1]

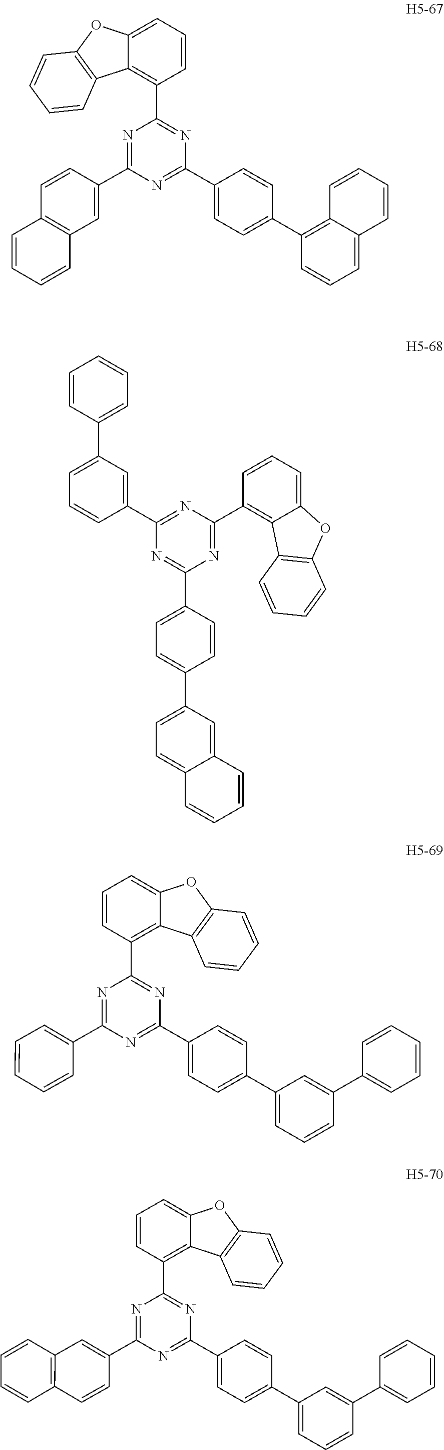 [SMILES: c1ccc2cc(-c3nc(-c4ccc(-c5cccc6ccccc56)cc4)nc(-c4cccc5oc6ccccc6c45)n3)ccc2c1 ; c1ccc(-c2cccc(-c3nc(-c4ccc(-c5ccc6ccccc6c5)cc4)nc(-c4cccc5oc6ccccc6c45)n3)c2)cc1 ; c1ccc(-c2cccc(-c3ccc(-c4nc(-c5ccccc5)nc(-c5cccc6oc7ccccc7c56)n4)cc3)c2)cc1 ; c1ccc(-c2cccc(-c3ccc(-c4nc(-c5ccc6ccccc6c5)nc(-c5cccc6oc7ccccc7c56)n4)cc3)c2)cc1]

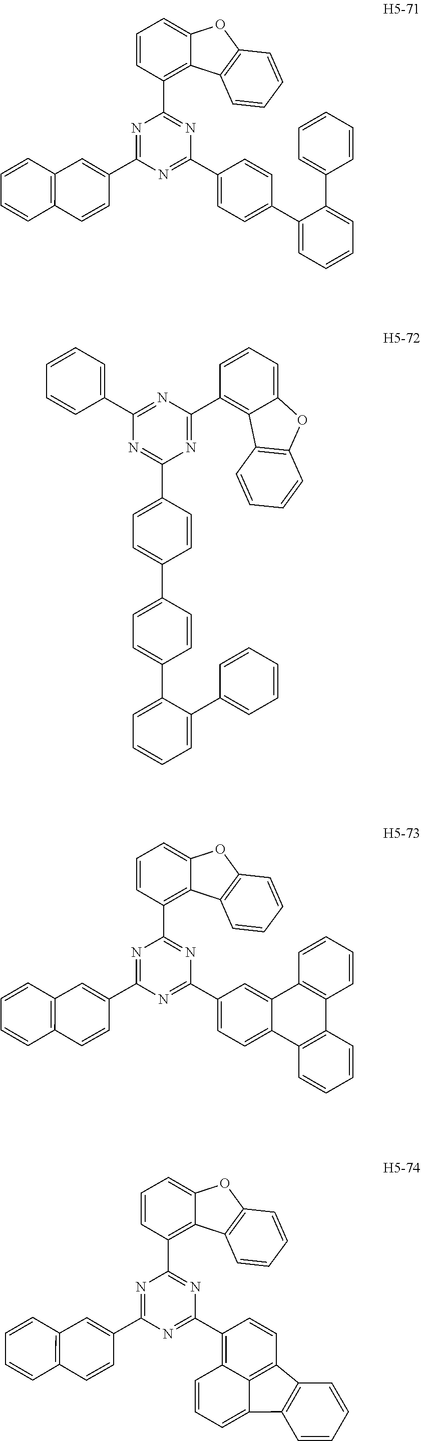 [SMILES: c1ccc(-c2ccccc2-c2ccc(-c3nc(-c4ccc5ccccc5c4)nc(-c4cccc5oc6ccccc6c45)n3)cc2)cc1 ; c1ccc(-c2nc(-c3ccc(-c4ccc(-c5ccccc5-c5ccccc5)cc4)cc3)nc(-c3cccc4oc5ccccc5c34)n2)cc1 ; c1ccc2cc(-c3nc(-c4ccc5c6ccccc6c6ccccc6c5c4)nc(-c4cccc5oc6ccccc6c45)n3)ccc2c1 ; c1ccc2c(c1)-c1cccc3c(-c4nc(-c5ccc6ccccc6c5)nc(-c5cccc6oc7ccccc7c56)n4)ccc-2c13]

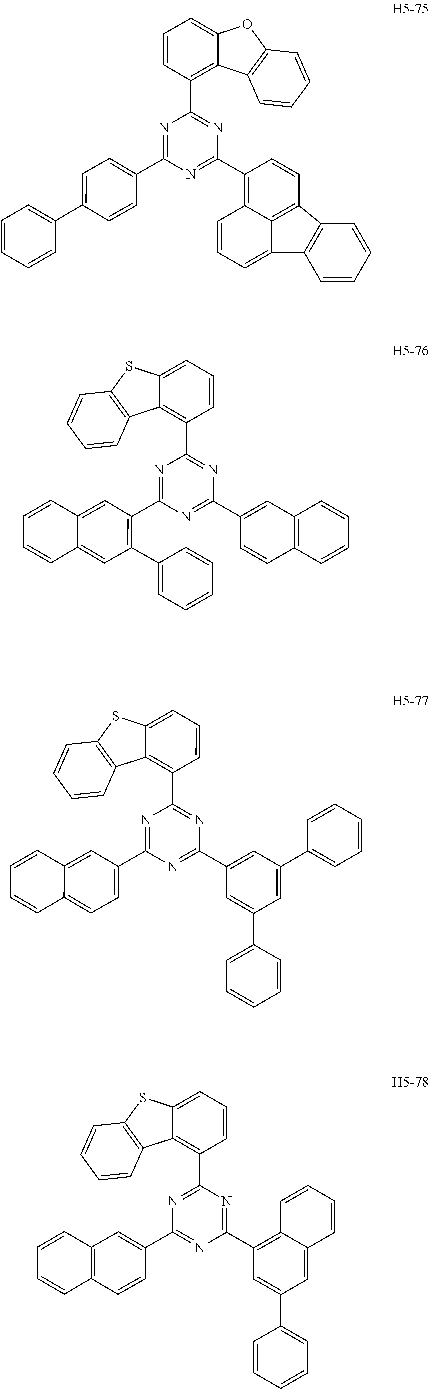 [SMILES: c1ccc(-c2ccc(-c3nc(-c4ccc5c6c(cccc46)-c4ccccc4-5)nc(-c4cccc5oc6ccccc6c45)n3)cc2)cc1 ; c1ccc(-c2cc3ccccc3cc2-c2nc(-c3ccc4ccccc4c3)nc(-c3cccc4sc5ccccc5c34)n2)cc1 ; c1ccc(-c2cc(-c3ccccc3)cc(-c3nc(-c4ccc5ccccc5c4)nc(-c4cccc5sc6ccccc6c45)n3)c2)cc1 ; c1ccc(-c2cc(-c3nc(-c4ccc5ccccc5c4)nc(-c4cccc5sc6ccccc6c45)n3)c3ccccc3c2)cc1]

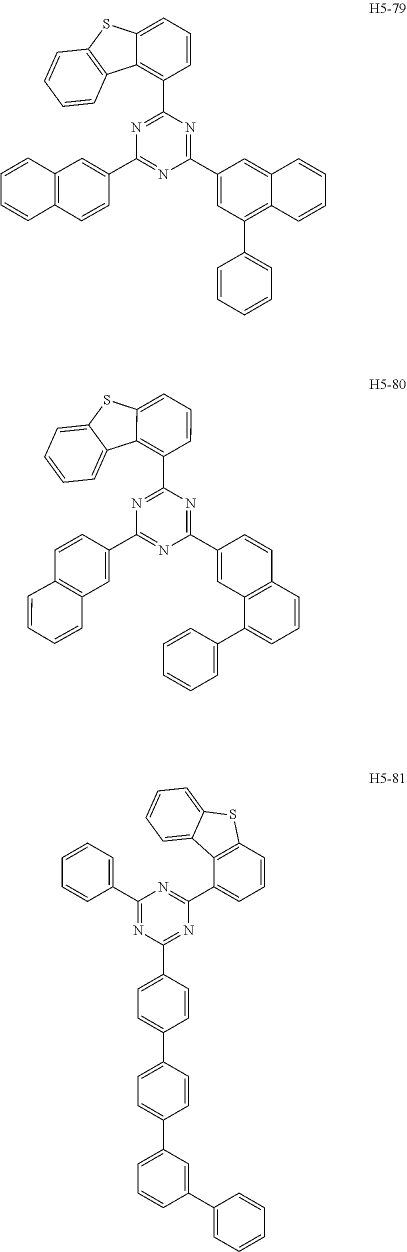 [SMILES: c1ccc(-c2cc(-c3nc(-c4ccc5ccccc5c4)nc(-c4cccc5sc6ccccc6c45)n3)cc3ccccc23)cc1 ; c1ccc(-c2cccc3ccc(-c4nc(-c5ccc6ccccc6c5)nc(-c5cccc6sc7ccccc7c56)n4)cc23)cc1 ; c1ccc(-c2cccc(-c3ccc(-c4ccc(-c5nc(-c6ccccc6)nc(-c6cccc7sc8ccccc8c67)n5)cc4)cc3)c2)cc1]

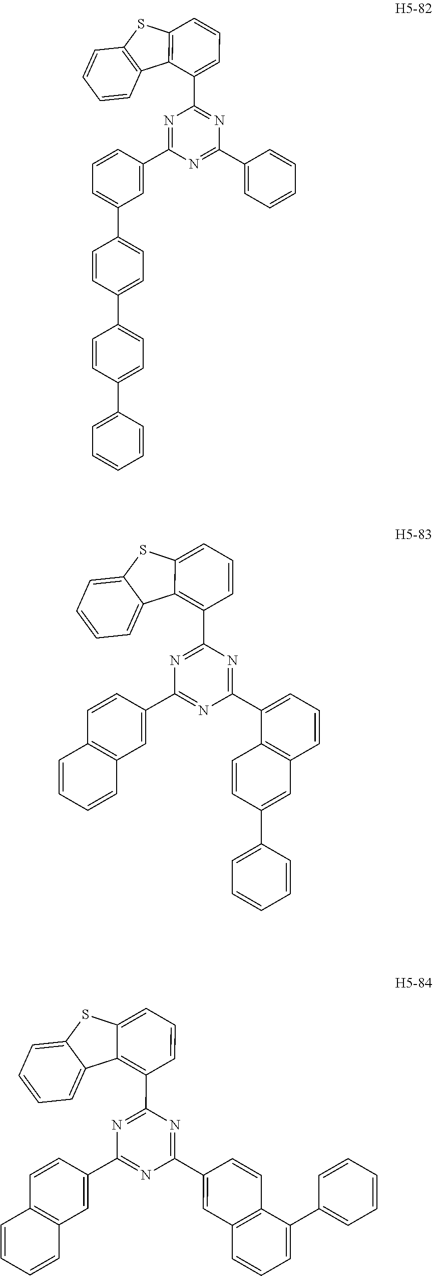 [SMILES: c1ccc(-c2ccc(-c3ccc(-c4cccc(-c5nc(-c6ccccc6)nc(-c6cccc7sc8ccccc8c67)n5)c4)cc3)cc2)cc1 ; c1ccc(-c2ccc3c(-c4nc(-c5ccc6ccccc6c5)nc(-c5cccc6sc7ccccc7c56)n4)cccc3c2)cc1 ; c1ccc(-c2cccc3cc(-c4nc(-c5ccc6ccccc6c5)nc(-c5cccc6sc7ccccc7c56)n4)ccc23)cc1]

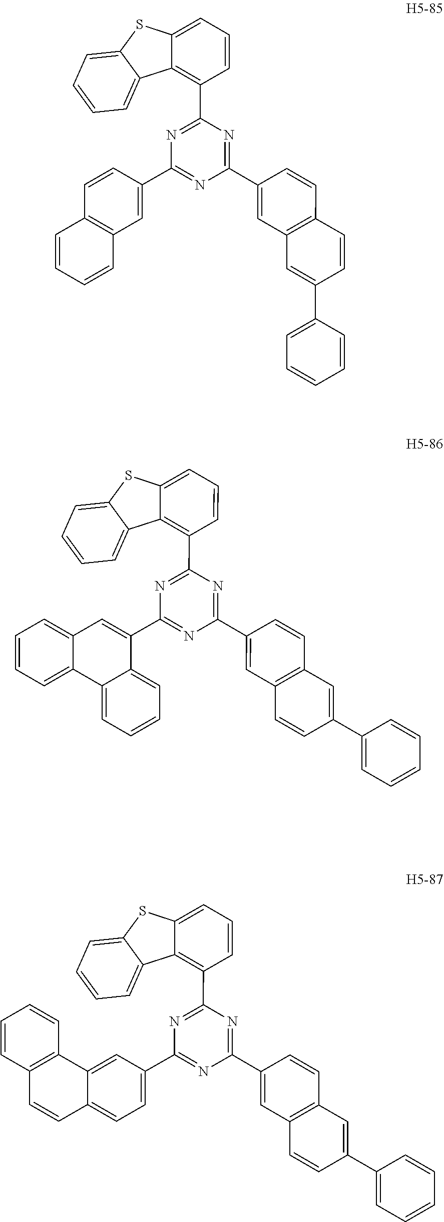 [SMILES: c1ccc(-c2ccc3ccc(-c4nc(-c5ccc6ccccc6c5)nc(-c5cccc6sc7ccccc7c56)n4)cc3c2)cc1 ; c1ccc(-c2ccc3cc(-c4nc(-c5cc6ccccc6c6ccccc56)nc(-c5cccc6sc7ccccc7c56)n4)ccc3c2)cc1 ; c1ccc(-c2ccc3cc(-c4nc(-c5ccc6ccc7ccccc7c6c5)nc(-c5cccc6sc7ccccc7c56)n4)ccc3c2)cc1]

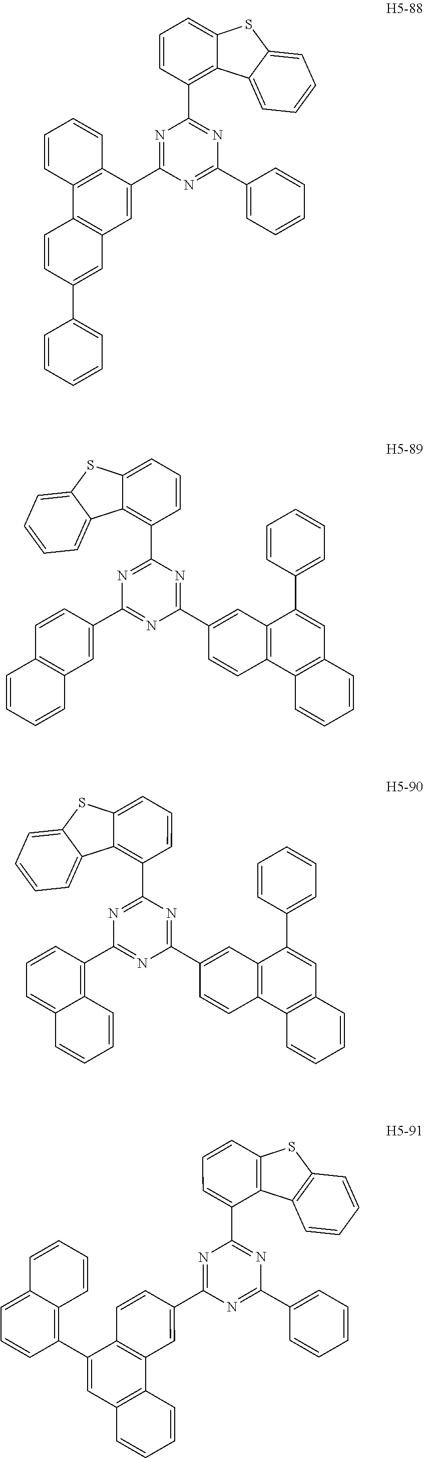 [SMILES: c1ccc(-c2ccc3c(c2)cc(-c2nc(-c4ccccc4)nc(-c4cccc5sc6ccccc6c45)n2)c2ccccc23)cc1 ; c1ccc(-c2cc3ccccc3c3ccc(-c4nc(-c5ccc6ccccc6c5)nc(-c5cccc6sc7ccccc7c56)n4)cc23)cc1 ; c1ccc(-c2cc3ccccc3c3ccc(-c4nc(-c5cccc6ccccc56)nc(-c5cccc6sc7ccccc7c56)n4)cc23)cc1 ; c1ccc(-c2nc(-c3ccc4c(-c5cccc6ccccc56)cc5ccccc5c4c3)nc(-c3cccc4sc5ccccc5c34)n2)cc1]

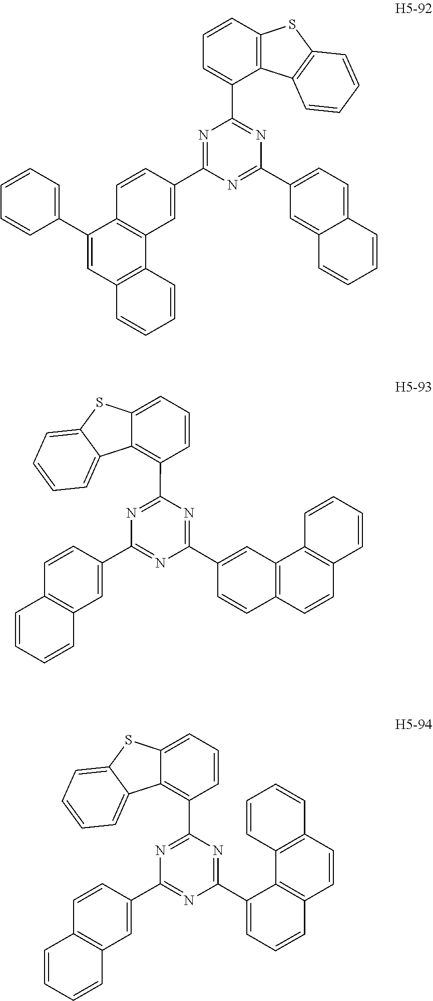 [SMILES: c1ccc(-c2cc3ccccc3c3cc(-c4nc(-c5ccc6ccccc6c5)nc(-c5cccc6sc7ccccc7c56)n4)ccc23)cc1 ; c1ccc2cc(-c3nc(-c4ccc5ccc6ccccc6c5c4)nc(-c4cccc5sc6ccccc6c45)n3)ccc2c1 ; c1ccc2cc(-c3nc(-c4cccc5ccc6ccccc6c45)nc(-c4cccc5sc6ccccc6c45)n3)ccc2c1]

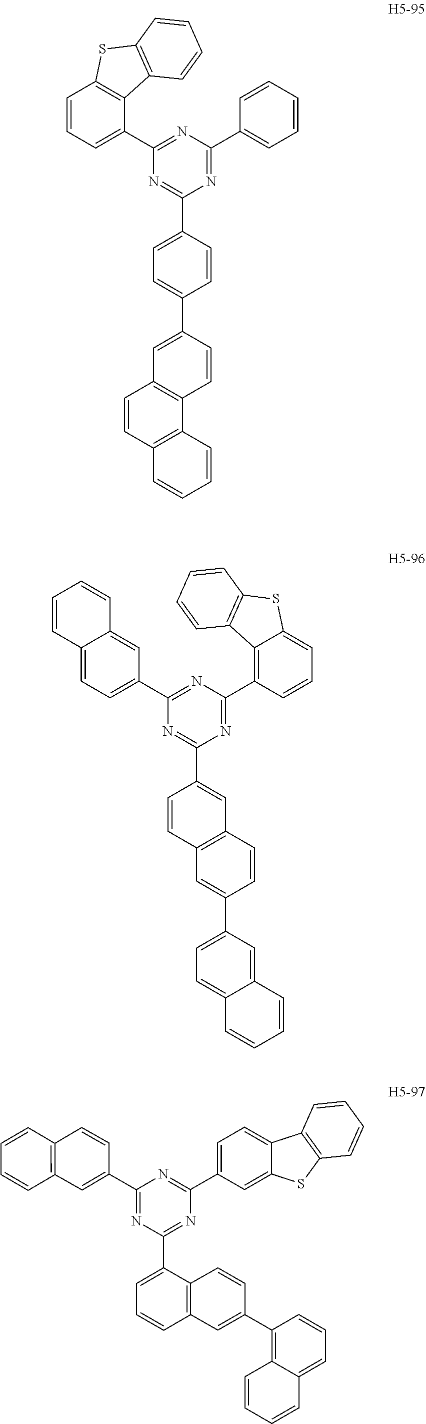 [SMILES: c1ccc(-c2nc(-c3ccc(-c4ccc5c(ccc6ccccc65)c4)cc3)nc(-c3cccc4sc5ccccc5c34)n2)cc1 ; c1ccc2cc(-c3ccc4cc(-c5nc(-c6ccc7ccccc7c6)nc(-c6cccc7sc8ccccc8c67)n5)ccc4c3)ccc2c1 ; c1ccc2cc(-c3nc(-c4ccc5c(c4)sc4ccccc45)nc(-c4cccc5cc(-c6cccc7ccccc67)ccc45)n3)ccc2c1]

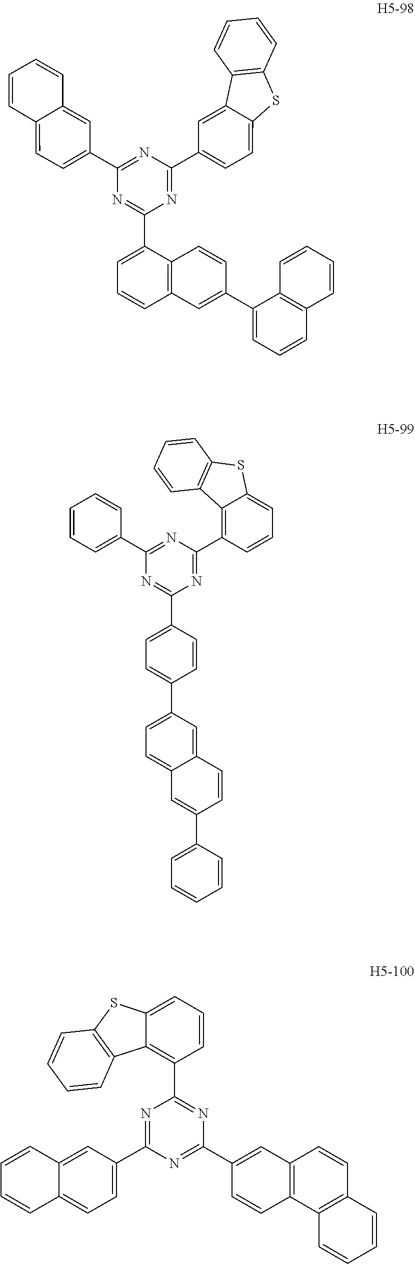 [SMILES: c1ccc2cc(-c3nc(-c4ccc5sc6ccccc6c5c4)nc(-c4cccc5cc(-c6cccc7ccccc67)ccc45)n3)ccc2c1 ; c1ccc(-c2ccc3cc(-c4ccc(-c5nc(-c6ccccc6)nc(-c6cccc7sc8ccccc8c67)n5)cc4)ccc3c2)cc1 ; c1ccc2cc(-c3nc(-c4ccc5c(ccc6ccccc65)c4)nc(-c4cccc5sc6ccccc6c45)n3)ccc2c1]

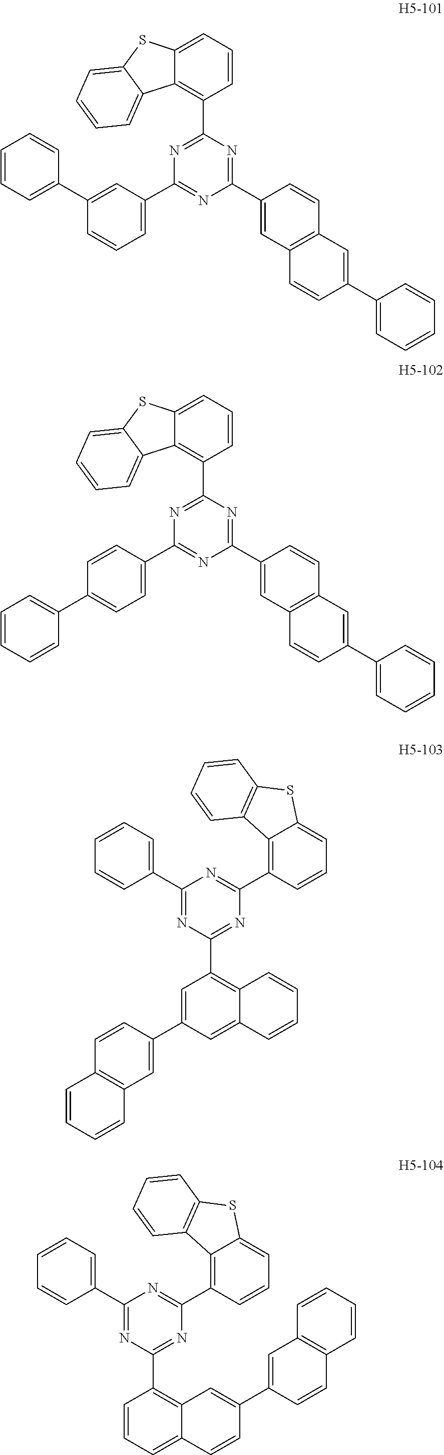 [SMILES: c1ccc(-c2cccc(-c3nc(-c4ccc5cc(-c6ccccc6)ccc5c4)nc(-c4cccc5sc6ccccc6c45)n3)c2)cc1 ; c1ccc(-c2ccc(-c3nc(-c4ccc5cc(-c6ccccc6)ccc5c4)nc(-c4cccc5sc6ccccc6c45)n3)cc2)cc1 ; c1ccc(-c2nc(-c3cc(-c4ccc5ccccc5c4)cc4ccccc34)nc(-c3cccc4sc5ccccc5c34)n2)cc1 ; c1ccc(-c2nc(-c3cccc4ccc(-c5ccc6ccccc6c5)cc34)nc(-c3cccc4sc5ccccc5c34)n2)cc1]

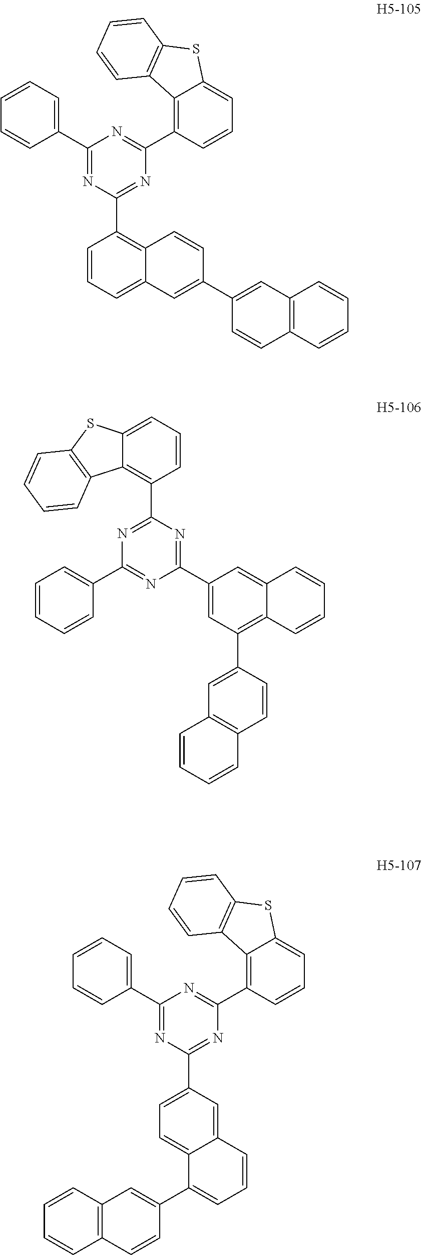 [SMILES: c1ccc(-c2nc(-c3cccc4cc(-c5ccc6ccccc6c5)ccc34)nc(-c3cccc4sc5ccccc5c34)n2)cc1 ; c1ccc(-c2nc(-c3cc(-c4ccc5ccccc5c4)c4ccccc4c3)nc(-c3cccc4sc5ccccc5c34)n2)cc1 ; c1ccc(-c2nc(-c3ccc4c(-c5ccc6ccccc6c5)cccc4c3)nc(-c3cccc4sc5ccccc5c34)n2)cc1]

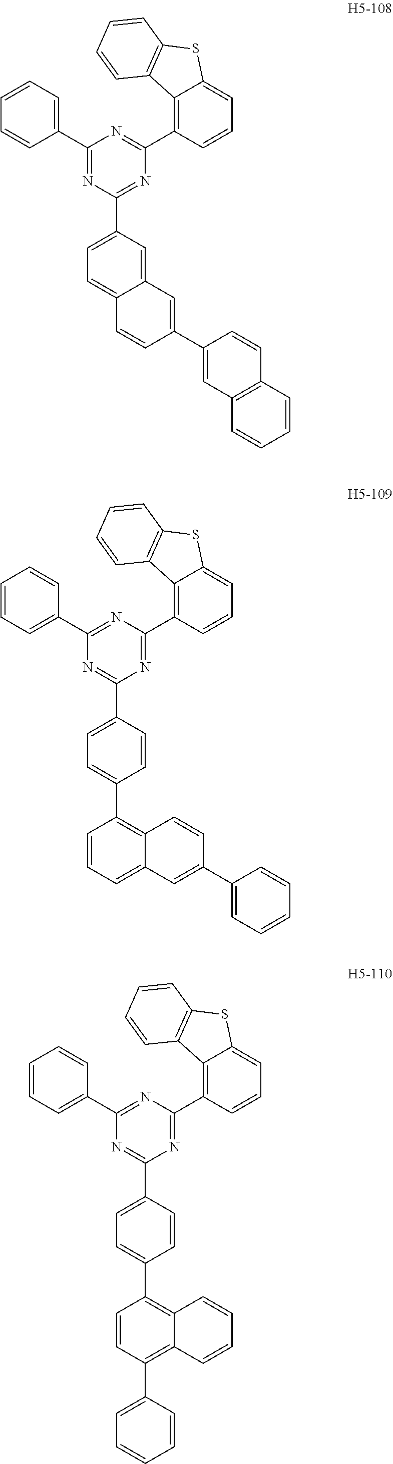 [SMILES: c1ccc(-c2nc(-c3ccc4ccc(-c5ccc6ccccc6c5)cc4c3)nc(-c3cccc4sc5ccccc5c34)n2)cc1 ; c1ccc(-c2ccc3c(-c4ccc(-c5nc(-c6ccccc6)nc(-c6cccc7sc8ccccc8c67)n5)cc4)cccc3c2)cc1 ; c1ccc(-c2nc(-c3ccc(-c4ccc(-c5ccccc5)c5ccccc45)cc3)nc(-c3cccc4sc5ccccc5c34)n2)cc1]

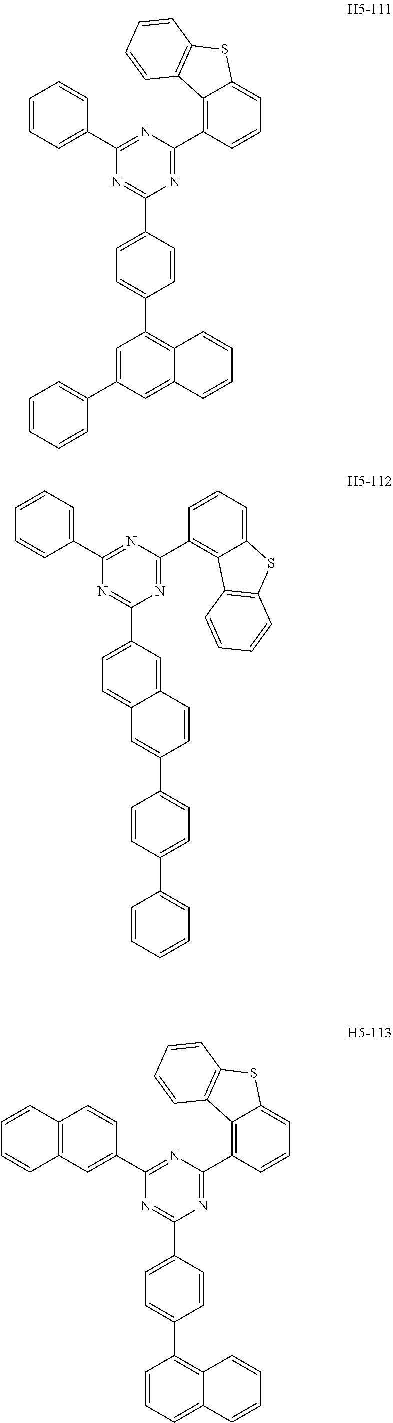

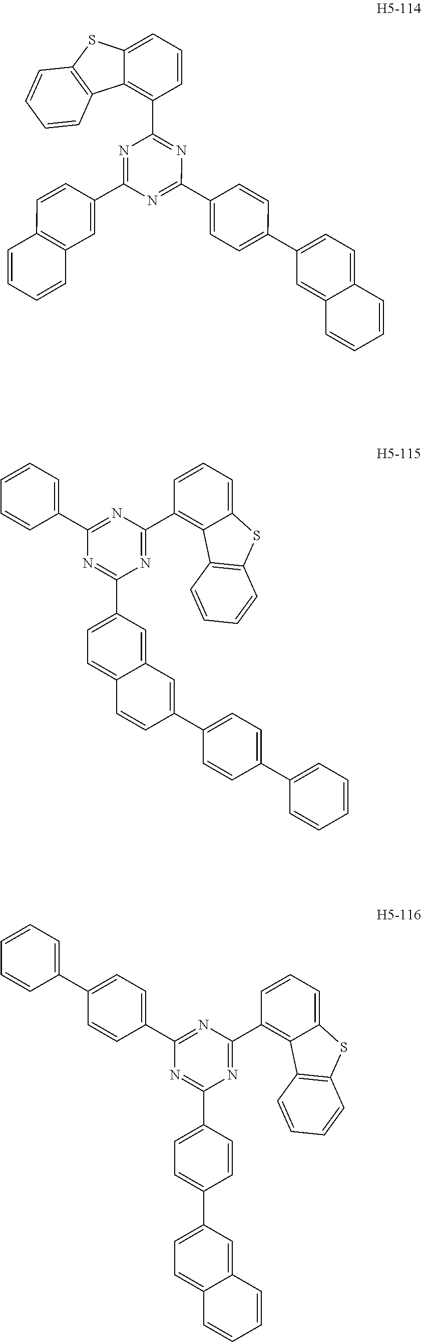 [SMILES: c1ccc2cc(-c3ccc(-c4nc(-c5ccc6ccccc6c5)nc(-c5cccc6sc7ccccc7c56)n4)cc3)ccc2c1 ; c1ccc(-c2ccc(-c3ccc4ccc(-c5nc(-c6ccccc6)nc(-c6cccc7sc8ccccc8c67)n5)cc4c3)cc2)cc1 ; c1ccc(-c2ccc(-c3nc(-c4ccc(-c5ccc6ccccc6c5)cc4)nc(-c4cccc5sc6ccccc6c45)n3)cc2)cc1]

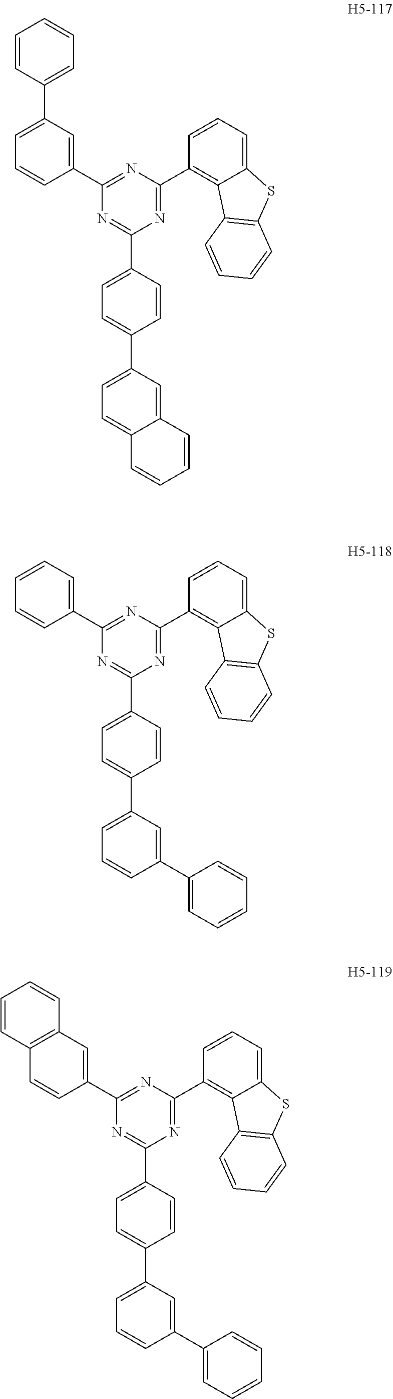 [SMILES: c1ccc(-c2cccc(-c3nc(-c4ccc(-c5ccc6ccccc6c5)cc4)nc(-c4cccc5sc6ccccc6c45)n3)c2)cc1 ; c1ccc(-c2cccc(-c3ccc(-c4nc(-c5ccccc5)nc(-c5cccc6sc7ccccc7c56)n4)cc3)c2)cc1 ; c1ccc(-c2cccc(-c3ccc(-c4nc(-c5ccc6ccccc6c5)nc(-c5cccc6sc7ccccc7c56)n4)cc3)c2)cc1]

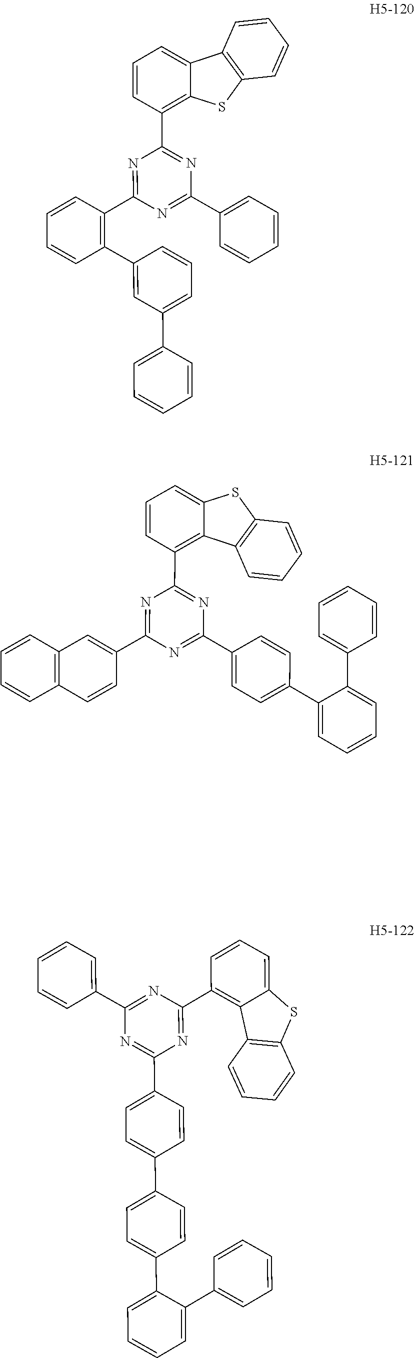 [SMILES: c1ccc(-c2cccc(-c3ccccc3-c3nc(-c4ccccc4)nc(-c4cccc5c4sc4ccccc45)n3)c2)cc1 ; c1ccc(-c2ccccc2-c2ccc(-c3nc(-c4ccc5ccccc5c4)nc(-c4cccc5sc6ccccc6c45)n3)cc2)cc1 ; c1ccc(-c2nc(-c3ccc(-c4ccc(-c5ccccc5-c5ccccc5)cc4)cc3)nc(-c3cccc4sc5ccccc5c34)n2)cc1]

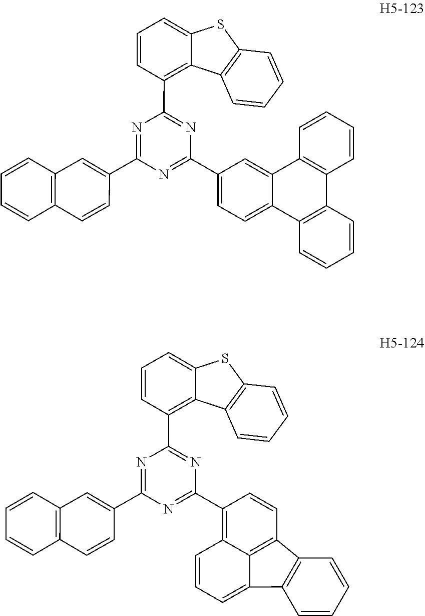 [SMILES: c1ccc2cc(-c3nc(-c4ccc5c6ccccc6c6ccccc6c5c4)nc(-c4cccc5sc6ccccc6c45)n3)ccc2c1 ; c1ccc2c(c1)-c1cccc3c(-c4nc(-c5ccc6ccccc6c5)nc(-c5cccc6sc7ccccc7c56)n4)ccc-2c13]

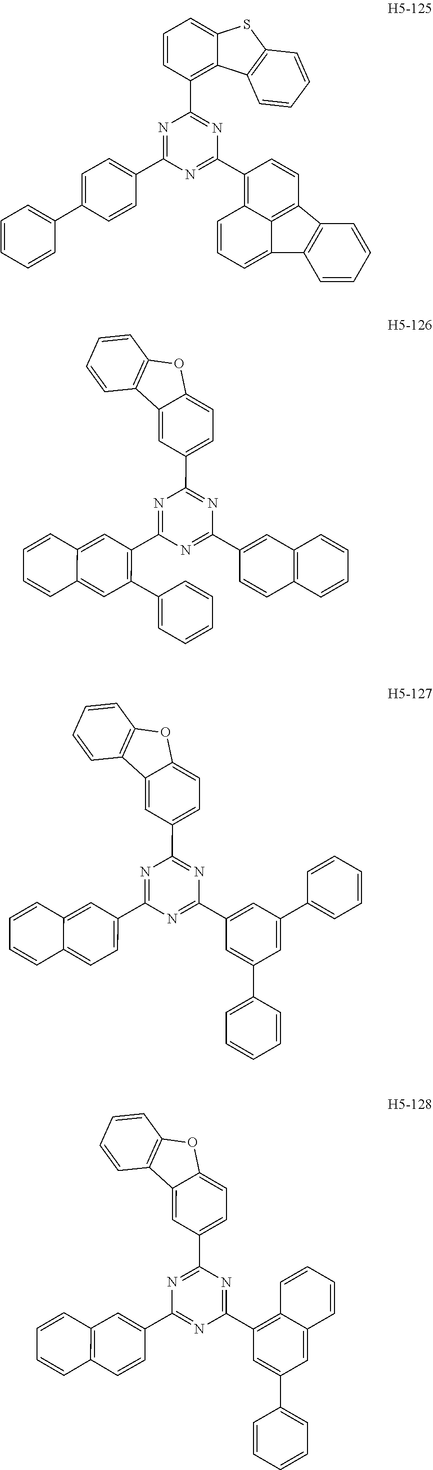 [SMILES: c1ccc(-c2ccc(-c3nc(-c4ccc5c6c(cccc46)-c4ccccc4-5)nc(-c4cccc5sc6ccccc6c45)n3)cc2)cc1 ; c1ccc(-c2cc3ccccc3cc2-c2nc(-c3ccc4ccccc4c3)nc(-c3ccc4oc5ccccc5c4c3)n2)cc1 ; c1ccc(-c2cc(-c3ccccc3)cc(-c3nc(-c4ccc5ccccc5c4)nc(-c4ccc5oc6ccccc6c5c4)n3)c2)cc1 ; c1ccc(-c2cc(-c3nc(-c4ccc5ccccc5c4)nc(-c4ccc5oc6ccccc6c5c4)n3)c3ccccc3c2)cc1]

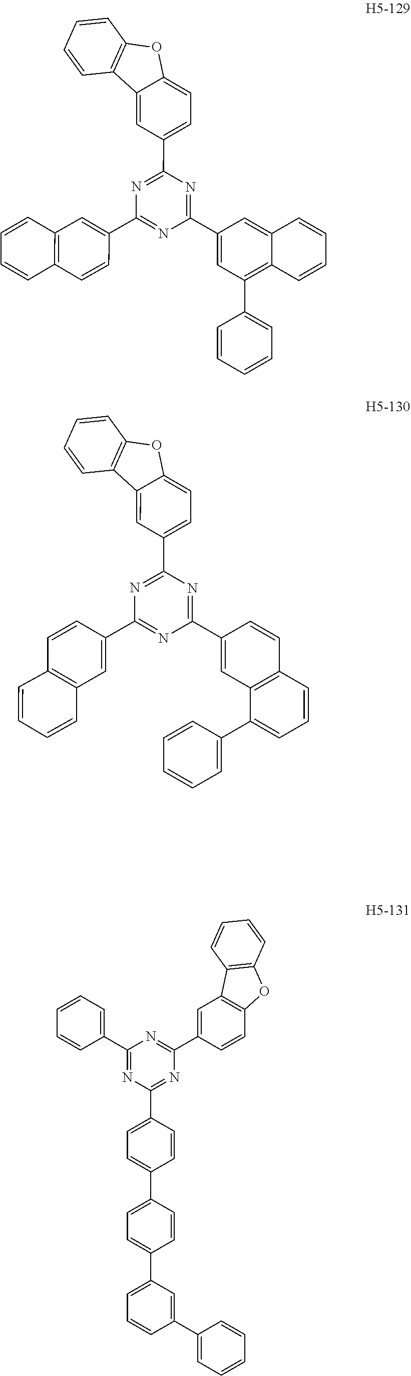 [SMILES: c1ccc(-c2cc(-c3nc(-c4ccc5ccccc5c4)nc(-c4ccc5oc6ccccc6c5c4)n3)cc3ccccc23)cc1 ; c1ccc(-c2cccc3ccc(-c4nc(-c5ccc6ccccc6c5)nc(-c5ccc6oc7ccccc7c6c5)n4)cc23)cc1 ; c1ccc(-c2cccc(-c3ccc(-c4ccc(-c5nc(-c6ccccc6)nc(-c6ccc7oc8ccccc8c7c6)n5)cc4)cc3)c2)cc1]

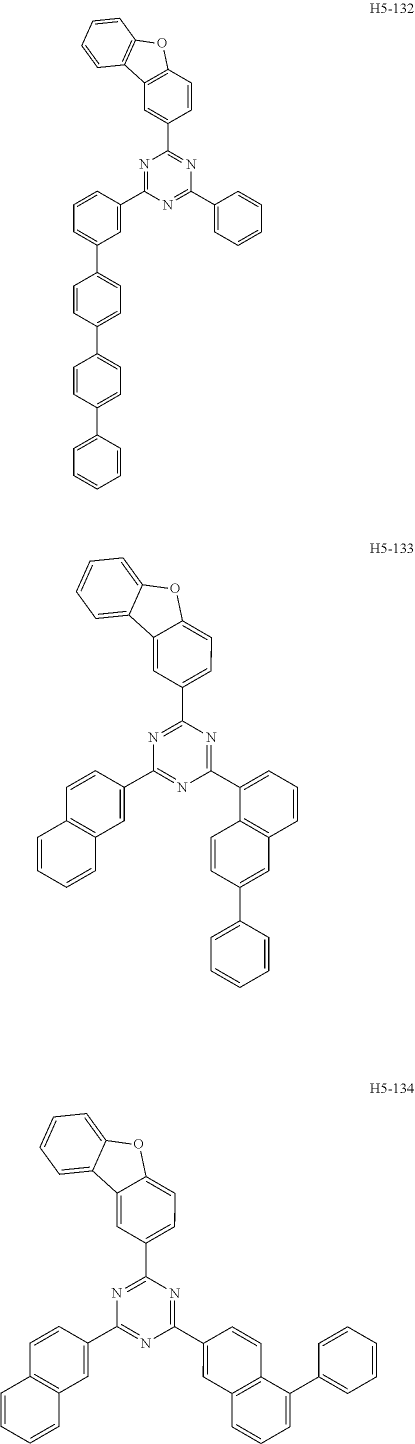 [SMILES: c1ccc(-c2ccc(-c3ccc(-c4cccc(-c5nc(-c6ccccc6)nc(-c6ccc7oc8ccccc8c7c6)n5)c4)cc3)cc2)cc1 ; c1ccc(-c2ccc3c(-c4nc(-c5ccc6ccccc6c5)nc(-c5ccc6oc7ccccc7c6c5)n4)cccc3c2)cc1 ; c1ccc(-c2cccc3cc(-c4nc(-c5ccc6ccccc6c5)nc(-c5ccc6oc7ccccc7c6c5)n4)ccc23)cc1]

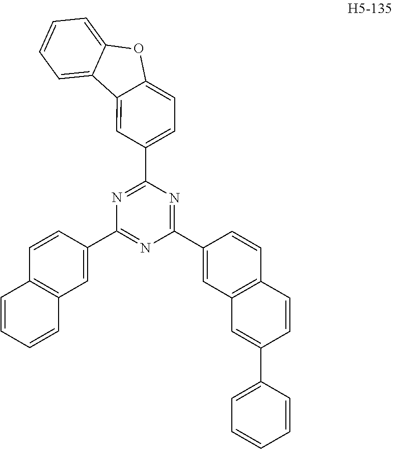 [SMILES: c1ccc(-c2ccc3ccc(-c4nc(-c5ccc6ccccc6c5)nc(-c5ccc6oc7ccccc7c6c5)n4)cc3c2)cc1]

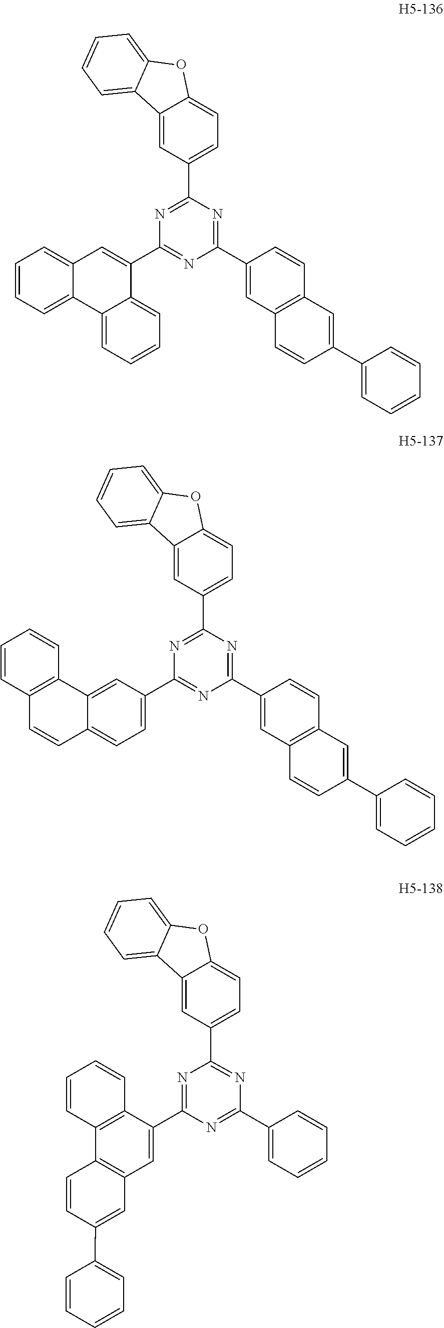

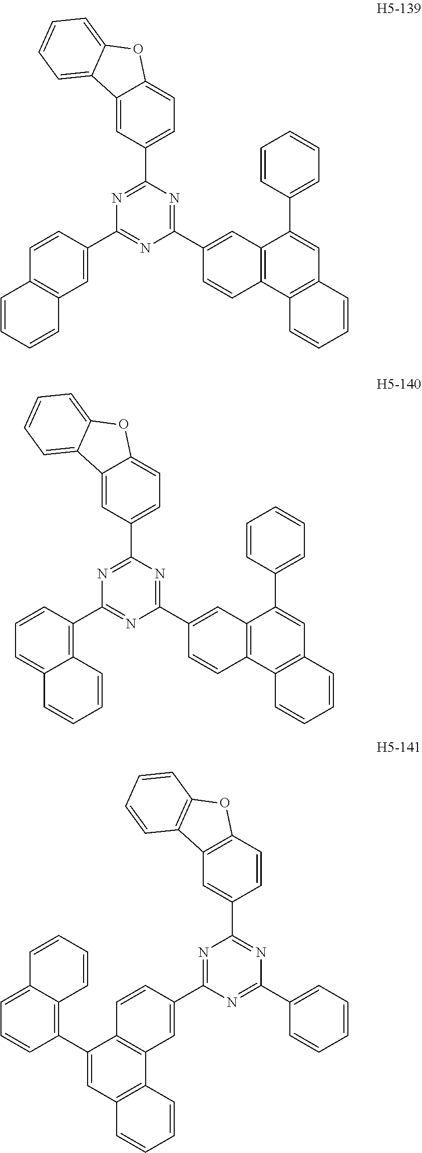 [SMILES: c1ccc(-c2cc3ccccc3c3ccc(-c4nc(-c5ccc6ccccc6c5)nc(-c5ccc6oc7ccccc7c6c5)n4)cc23)cc1 ; c1ccc(-c2cc3ccccc3c3ccc(-c4nc(-c5ccc6oc7ccccc7c6c5)nc(-c5cccc6ccccc56)n4)cc23)cc1 ; c1ccc(-c2nc(-c3ccc4oc5ccccc5c4c3)nc(-c3ccc4c(-c5cccc6ccccc56)cc5ccccc5c4c3)n2)cc1]

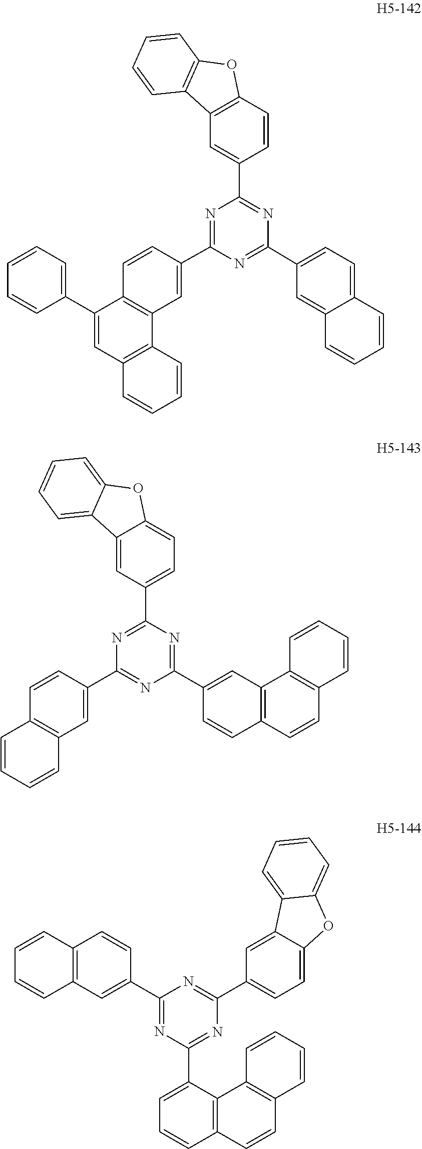 [SMILES: c1ccc(-c2cc3ccccc3c3cc(-c4nc(-c5ccc6ccccc6c5)nc(-c5ccc6oc7ccccc7c6c5)n4)ccc23)cc1 ; c1ccc2cc(-c3nc(-c4ccc5ccc6ccccc6c5c4)nc(-c4ccc5oc6ccccc6c5c4)n3)ccc2c1 ; c1ccc2cc(-c3nc(-c4ccc5oc6ccccc6c5c4)nc(-c4cccc5ccc6ccccc6c45)n3)ccc2c1]

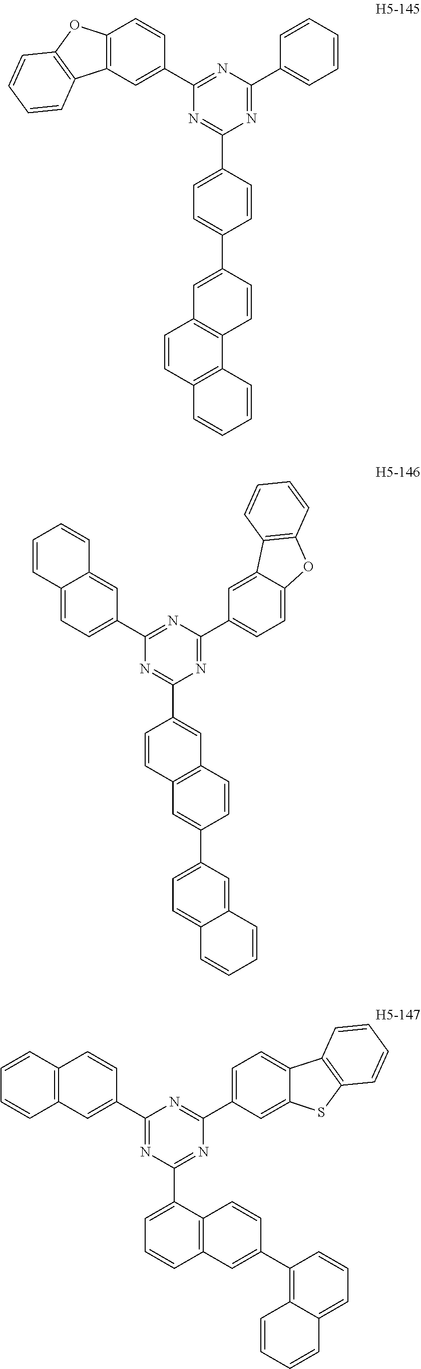 [SMILES: c1ccc(-c2nc(-c3ccc(-c4ccc5c(ccc6ccccc65)c4)cc3)nc(-c3ccc4oc5ccccc5c4c3)n2)cc1 ; c1ccc2cc(-c3ccc4cc(-c5nc(-c6ccc7ccccc7c6)nc(-c6ccc7oc8ccccc8c7c6)n5)ccc4c3)ccc2c1 ; c1ccc2cc(-c3nc(-c4ccc5c(c4)sc4ccccc45)nc(-c4cccc5cc(-c6cccc7ccccc67)ccc45)n3)ccc2c1]

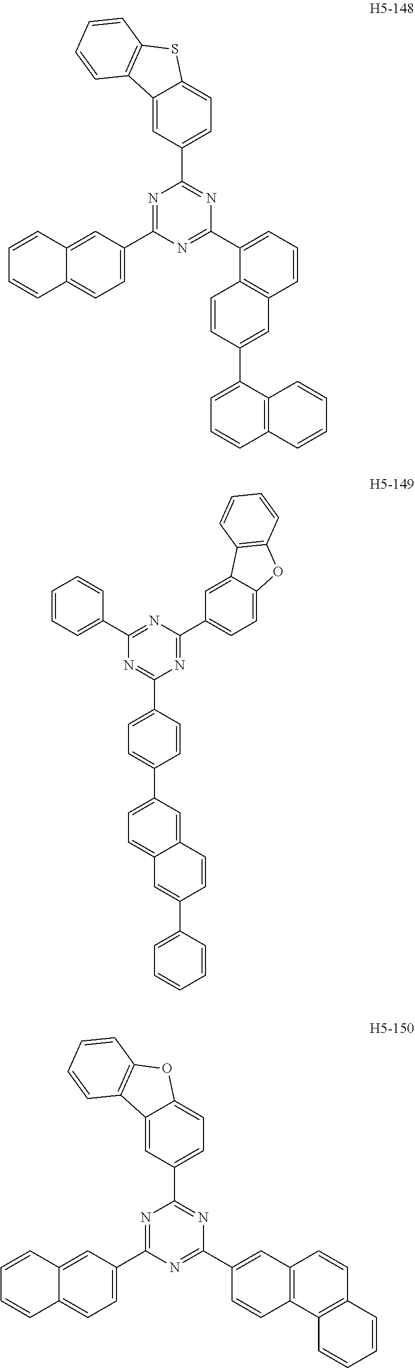 [SMILES: c1ccc2cc(-c3nc(-c4ccc5sc6ccccc6c5c4)nc(-c4cccc5cc(-c6cccc7ccccc67)ccc45)n3)ccc2c1 ; c1ccc(-c2ccc3cc(-c4ccc(-c5nc(-c6ccccc6)nc(-c6ccc7oc8ccccc8c7c6)n5)cc4)ccc3c2)cc1 ; c1ccc2cc(-c3nc(-c4ccc5c(ccc6ccccc65)c4)nc(-c4ccc5oc6ccccc6c5c4)n3)ccc2c1]

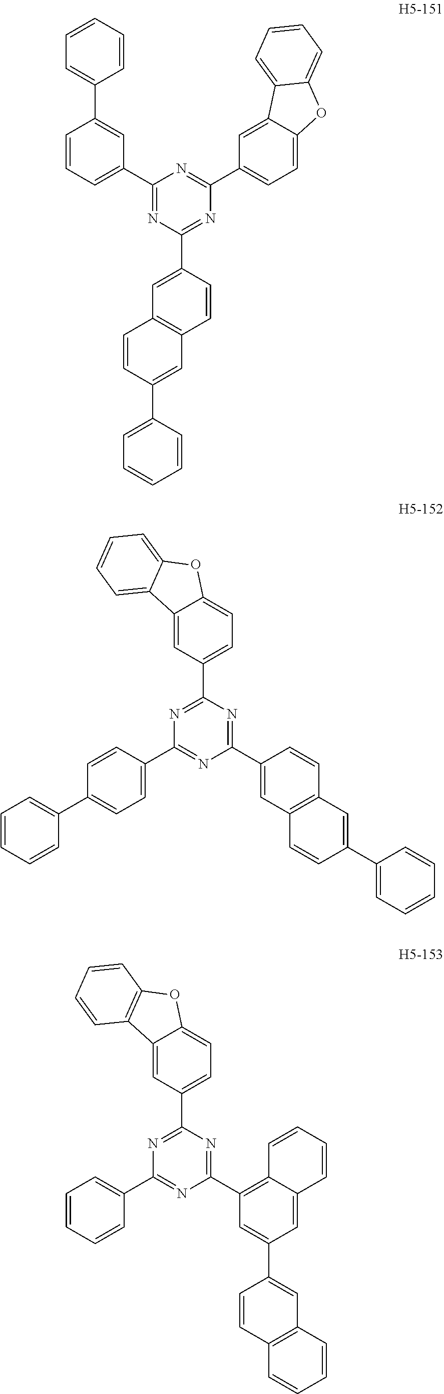 [SMILES: c1ccc(-c2cccc(-c3nc(-c4ccc5cc(-c6ccccc6)ccc5c4)nc(-c4ccc5oc6ccccc6c5c4)n3)c2)cc1 ; c1ccc(-c2ccc(-c3nc(-c4ccc5cc(-c6ccccc6)ccc5c4)nc(-c4ccc5oc6ccccc6c5c4)n3)cc2)cc1 ; c1ccc(-c2nc(-c3ccc4oc5ccccc5c4c3)nc(-c3cc(-c4ccc5ccccc5c4)cc4ccccc34)n2)cc1]

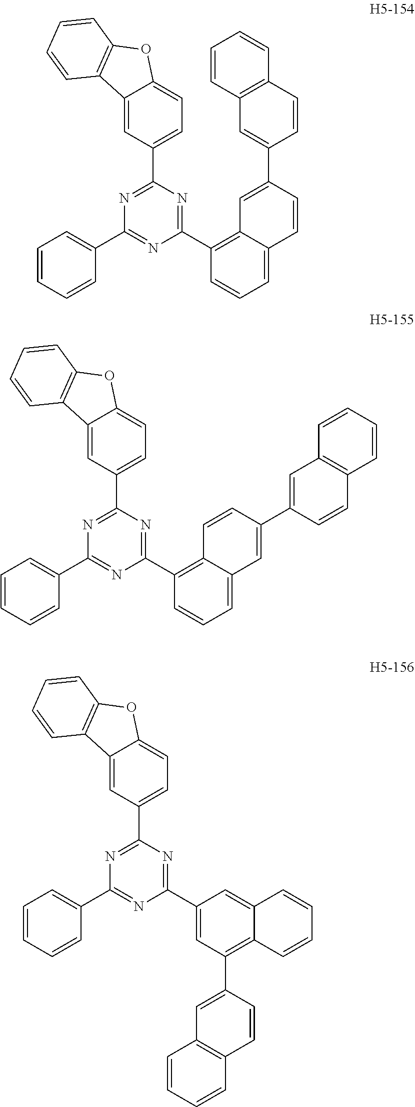 [SMILES: c1ccc(-c2nc(-c3ccc4oc5ccccc5c4c3)nc(-c3cccc4ccc(-c5ccc6ccccc6c5)cc34)n2)cc1 ; c1ccc(-c2nc(-c3ccc4oc5ccccc5c4c3)nc(-c3cccc4cc(-c5ccc6ccccc6c5)ccc34)n2)cc1 ; c1ccc(-c2nc(-c3cc(-c4ccc5ccccc5c4)c4ccccc4c3)nc(-c3ccc4oc5ccccc5c4c3)n2)cc1]

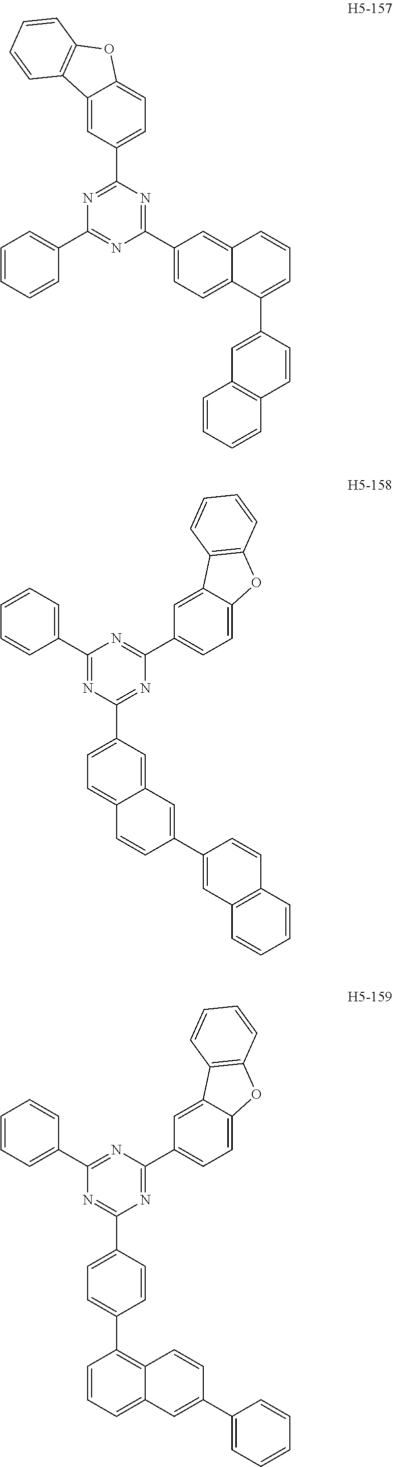 [SMILES: c1ccc(-c2nc(-c3ccc4c(-c5ccc6ccccc6c5)cccc4c3)nc(-c3ccc4oc5ccccc5c4c3)n2)cc1 ; c1ccc(-c2nc(-c3ccc4ccc(-c5ccc6ccccc6c5)cc4c3)nc(-c3ccc4oc5ccccc5c4c3)n2)cc1 ; c1ccc(-c2ccc3c(-c4ccc(-c5nc(-c6ccccc6)nc(-c6ccc7oc8ccccc8c7c6)n5)cc4)cccc3c2)cc1]

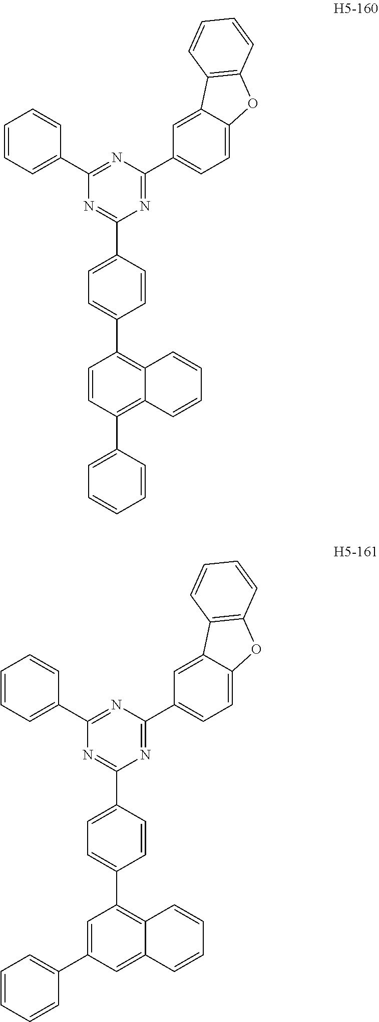 [SMILES: c1ccc(-c2nc(-c3ccc(-c4ccc(-c5ccccc5)c5ccccc45)cc3)nc(-c3ccc4oc5ccccc5c4c3)n2)cc1 ; c1ccc(-c2cc(-c3ccc(-c4nc(-c5ccccc5)nc(-c5ccc6oc7ccccc7c6c5)n4)cc3)c3ccccc3c2)cc1]

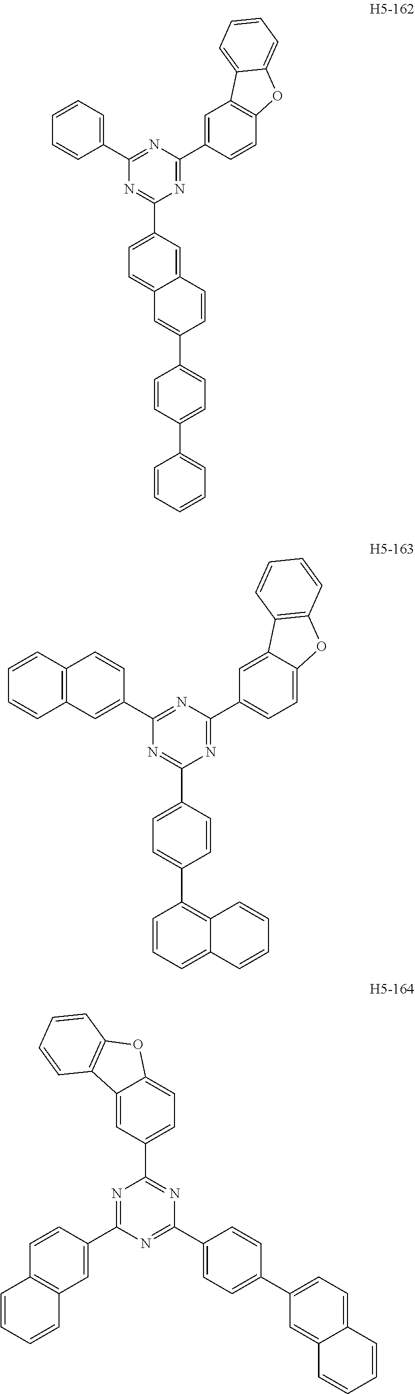 [SMILES: c1ccc(-c2ccc(-c3ccc4cc(-c5nc(-c6ccccc6)nc(-c6ccc7oc8ccccc8c7c6)n5)ccc4c3)cc2)cc1 ; c1ccc2cc(-c3nc(-c4ccc(-c5cccc6ccccc56)cc4)nc(-c4ccc5oc6ccccc6c5c4)n3)ccc2c1 ; c1ccc2cc(-c3ccc(-c4nc(-c5ccc6ccccc6c5)nc(-c5ccc6oc7ccccc7c6c5)n4)cc3)ccc2c1]

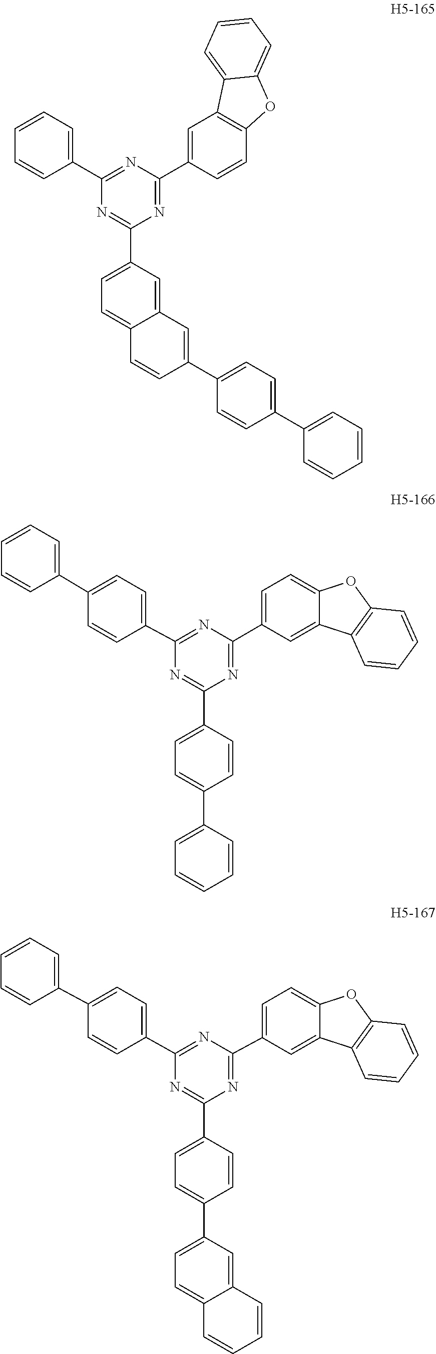 [SMILES: c1ccc(-c2ccc(-c3ccc4ccc(-c5nc(-c6ccccc6)nc(-c6ccc7oc8ccccc8c7c6)n5)cc4c3)cc2)cc1 ; c1ccc(-c2ccc(-c3nc(-c4ccc(-c5ccccc5)cc4)nc(-c4ccc5oc6ccccc6c5c4)n3)cc2)cc1 ; c1ccc(-c2ccc(-c3nc(-c4ccc(-c5ccc6ccccc6c5)cc4)nc(-c4ccc5oc6ccccc6c5c4)n3)cc2)cc1]

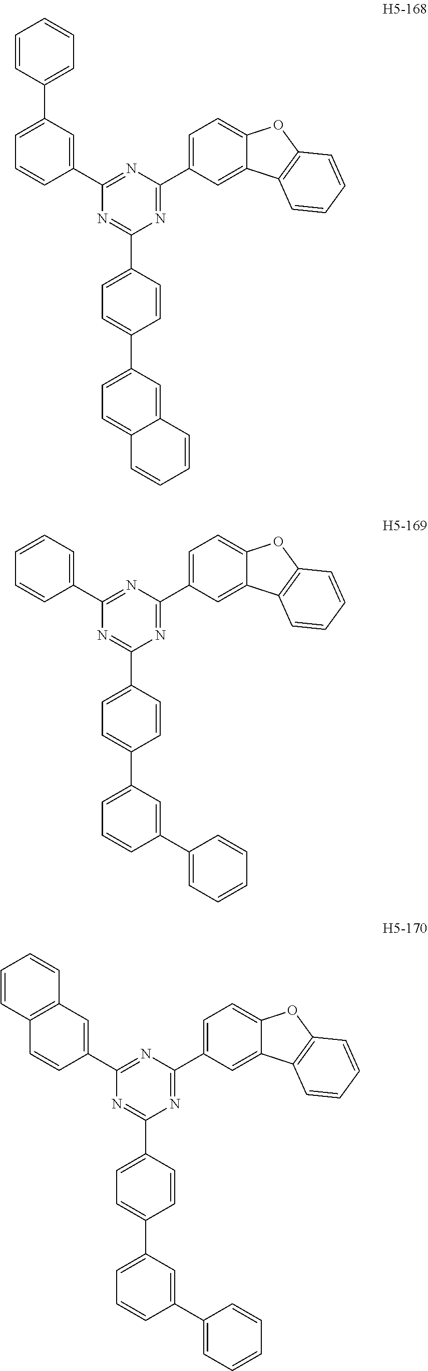 [SMILES: c1ccc(-c2cccc(-c3nc(-c4ccc(-c5ccc6ccccc6c5)cc4)nc(-c4ccc5oc6ccccc6c5c4)n3)c2)cc1 ; c1ccc(-c2cccc(-c3ccc(-c4nc(-c5ccccc5)nc(-c5ccc6oc7ccccc7c6c5)n4)cc3)c2)cc1 ; c1ccc(-c2cccc(-c3ccc(-c4nc(-c5ccc6ccccc6c5)nc(-c5ccc6oc7ccccc7c6c5)n4)cc3)c2)cc1]

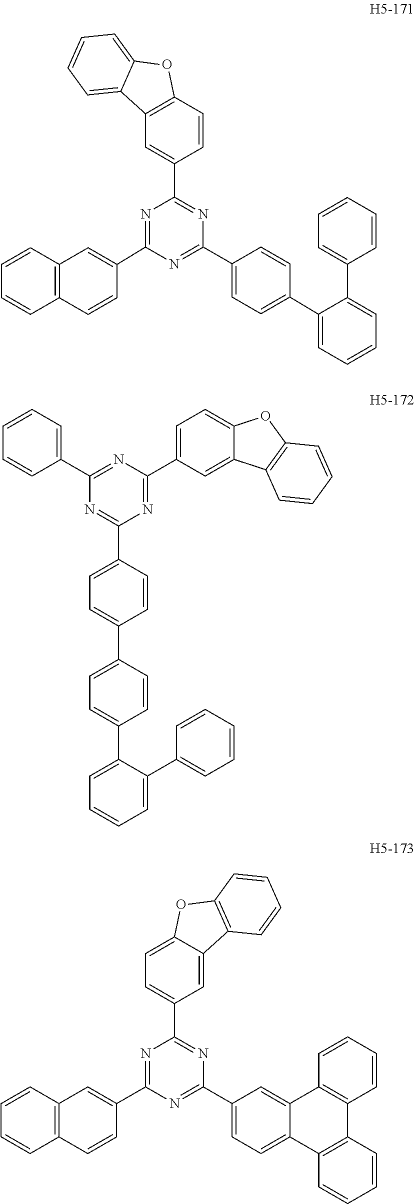 [SMILES: c1ccc(-c2ccccc2-c2ccc(-c3nc(-c4ccc5ccccc5c4)nc(-c4ccc5oc6ccccc6c5c4)n3)cc2)cc1 ; c1ccc(-c2nc(-c3ccc(-c4ccc(-c5ccccc5-c5ccccc5)cc4)cc3)nc(-c3ccc4oc5ccccc5c4c3)n2)cc1 ; c1ccc2cc(-c3nc(-c4ccc5oc6ccccc6c5c4)nc(-c4ccc5c6ccccc6c6ccccc6c5c4)n3)ccc2c1]

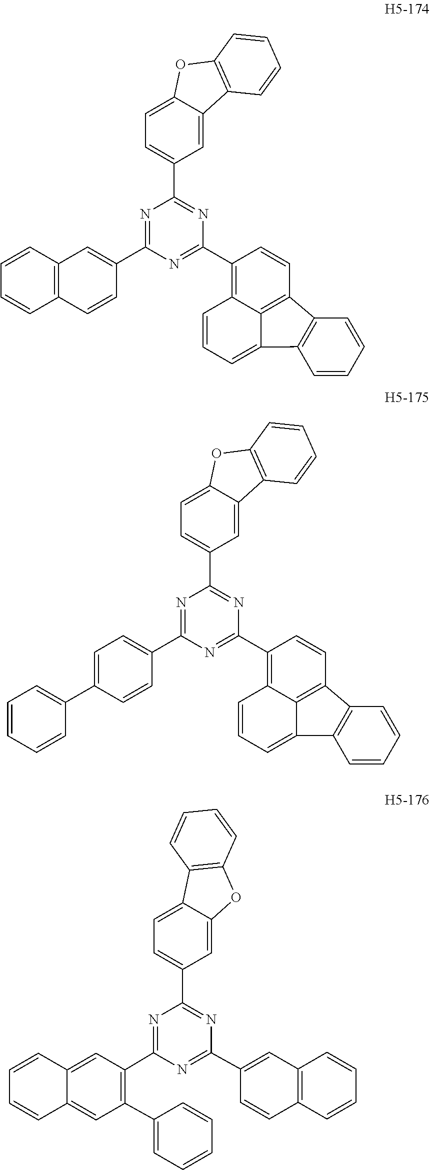 [SMILES: c1ccc2c(c1)-c1cccc3c(-c4nc(-c5ccc6ccccc6c5)nc(-c5ccc6oc7ccccc7c6c5)n4)ccc-2c13 ; c1ccc(-c2ccc(-c3nc(-c4ccc5oc6ccccc6c5c4)nc(-c4ccc5c6c(cccc46)-c4ccccc4-5)n3)cc2)cc1 ; c1ccc(-c2cc3ccccc3cc2-c2nc(-c3ccc4ccccc4c3)nc(-c3ccc4c(c3)oc3ccccc34)n2)cc1]

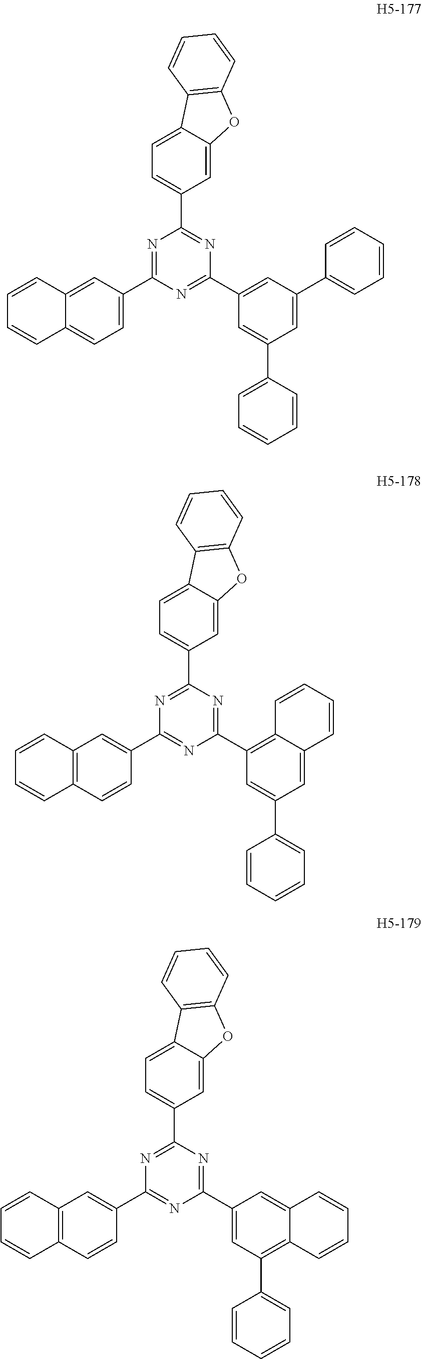 [SMILES: c1ccc(-c2cc(-c3ccccc3)cc(-c3nc(-c4ccc5ccccc5c4)nc(-c4ccc5c(c4)oc4ccccc45)n3)c2)cc1 ; c1ccc(-c2cc(-c3nc(-c4ccc5ccccc5c4)nc(-c4ccc5c(c4)oc4ccccc45)n3)c3ccccc3c2)cc1 ; c1ccc(-c2cc(-c3nc(-c4ccc5ccccc5c4)nc(-c4ccc5c(c4)oc4ccccc45)n3)cc3ccccc23)cc1]

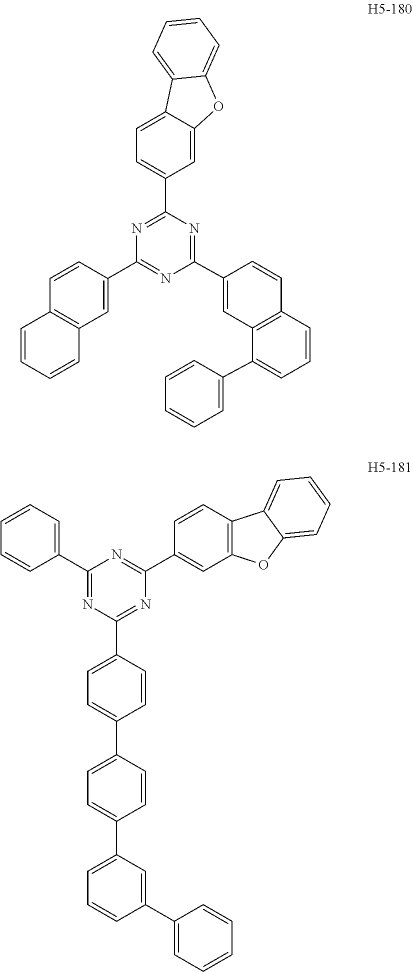 [SMILES: c1ccc(-c2cccc3ccc(-c4nc(-c5ccc6ccccc6c5)nc(-c5ccc6c(c5)oc5ccccc56)n4)cc23)cc1 ; c1ccc(-c2cccc(-c3ccc(-c4ccc(-c5nc(-c6ccccc6)nc(-c6ccc7c(c6)oc6ccccc67)n5)cc4)cc3)c2)cc1]

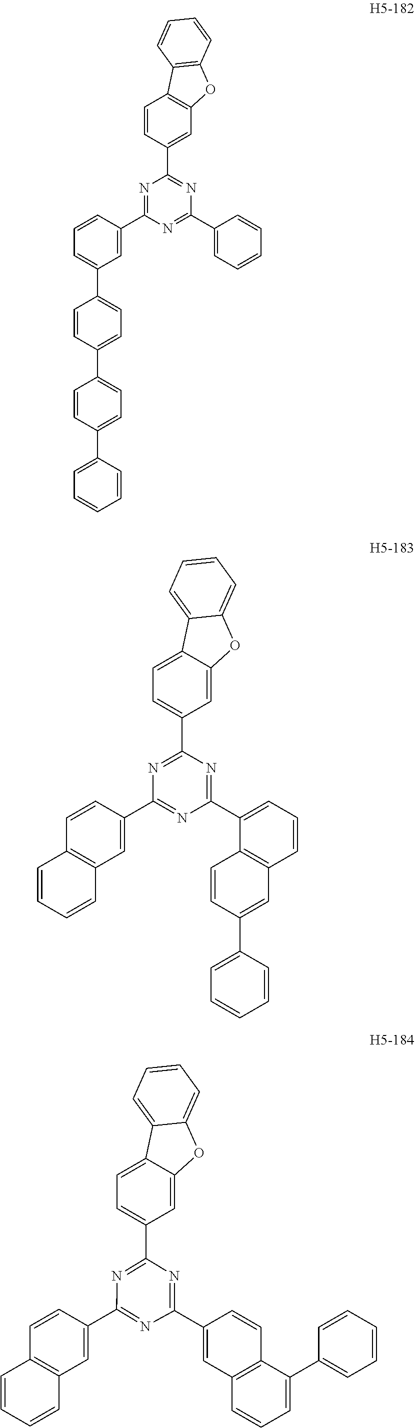

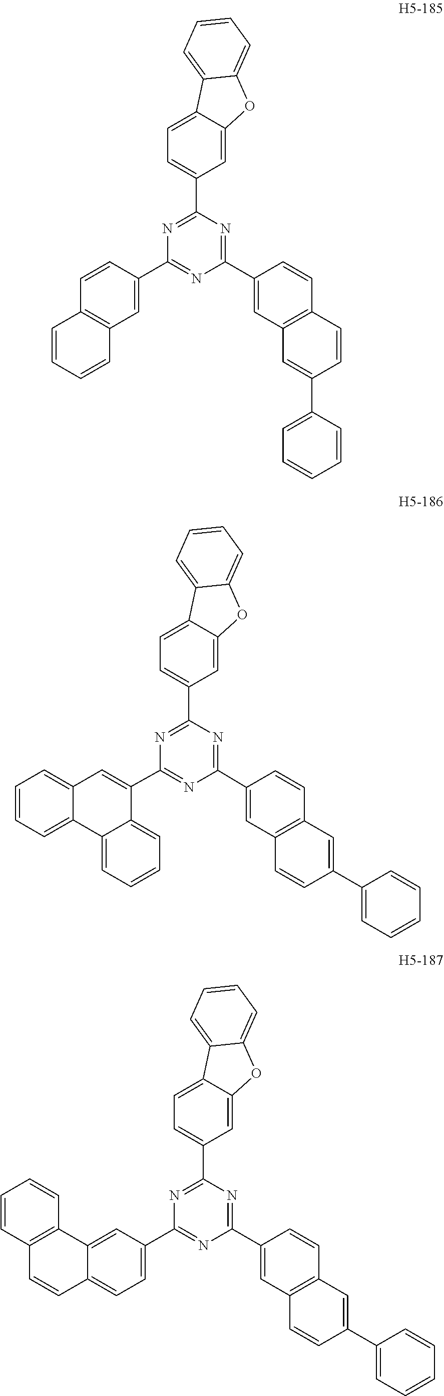 [SMILES: c1ccc(-c2ccc3ccc(-c4nc(-c5ccc6ccccc6c5)nc(-c5ccc6c(c5)oc5ccccc56)n4)cc3c2)cc1 ; c1ccc(-c2ccc3cc(-c4nc(-c5ccc6c(c5)oc5ccccc56)nc(-c5cc6ccccc6c6ccccc56)n4)ccc3c2)cc1 ; c1ccc(-c2ccc3cc(-c4nc(-c5ccc6c(c5)oc5ccccc56)nc(-c5ccc6ccc7ccccc7c6c5)n4)ccc3c2)cc1]

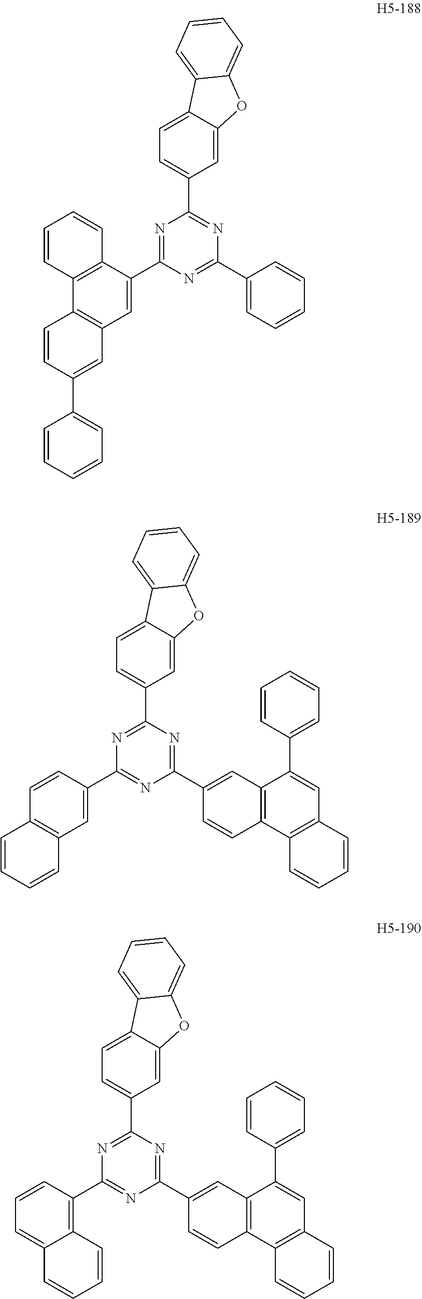 [SMILES: c1ccc(-c2ccc3c(c2)cc(-c2nc(-c4ccccc4)nc(-c4ccc5c(c4)oc4ccccc45)n2)c2ccccc23)cc1 ; c1ccc(-c2cc3ccccc3c3ccc(-c4nc(-c5ccc6ccccc6c5)nc(-c5ccc6c(c5)oc5ccccc56)n4)cc23)cc1 ; c1ccc(-c2cc3ccccc3c3ccc(-c4nc(-c5ccc6c(c5)oc5ccccc56)nc(-c5cccc6ccccc56)n4)cc23)cc1]

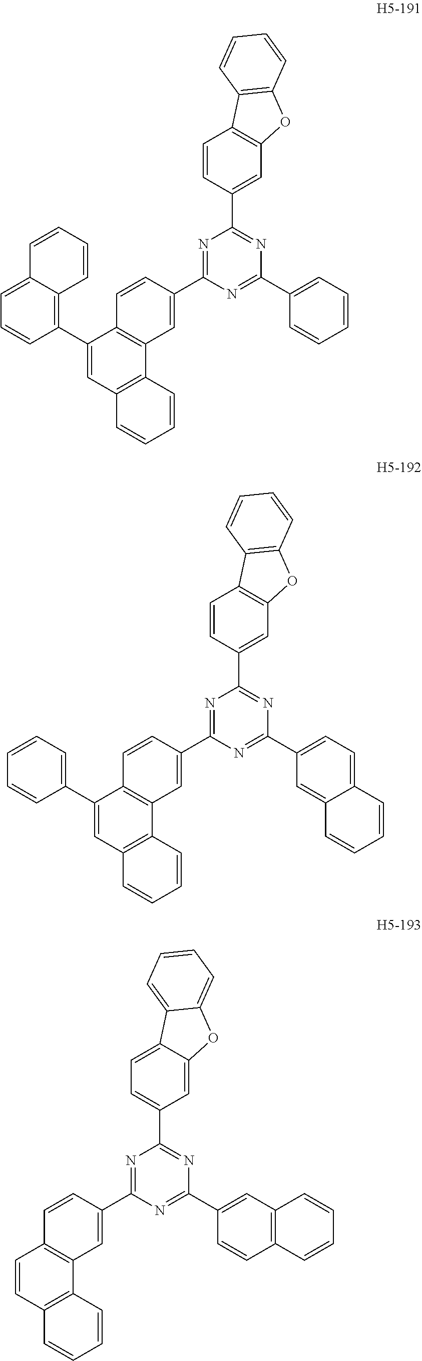 [SMILES: c1ccc(-c2nc(-c3ccc4c(c3)oc3ccccc34)nc(-c3ccc4c(-c5cccc6ccccc56)cc5ccccc5c4c3)n2)cc1 ; c1ccc(-c2cc3ccccc3c3cc(-c4nc(-c5ccc6ccccc6c5)nc(-c5ccc6c(c5)oc5ccccc56)n4)ccc23)cc1 ; c1ccc2cc(-c3nc(-c4ccc5c(c4)oc4ccccc45)nc(-c4ccc5ccc6ccccc6c5c4)n3)ccc2c1]

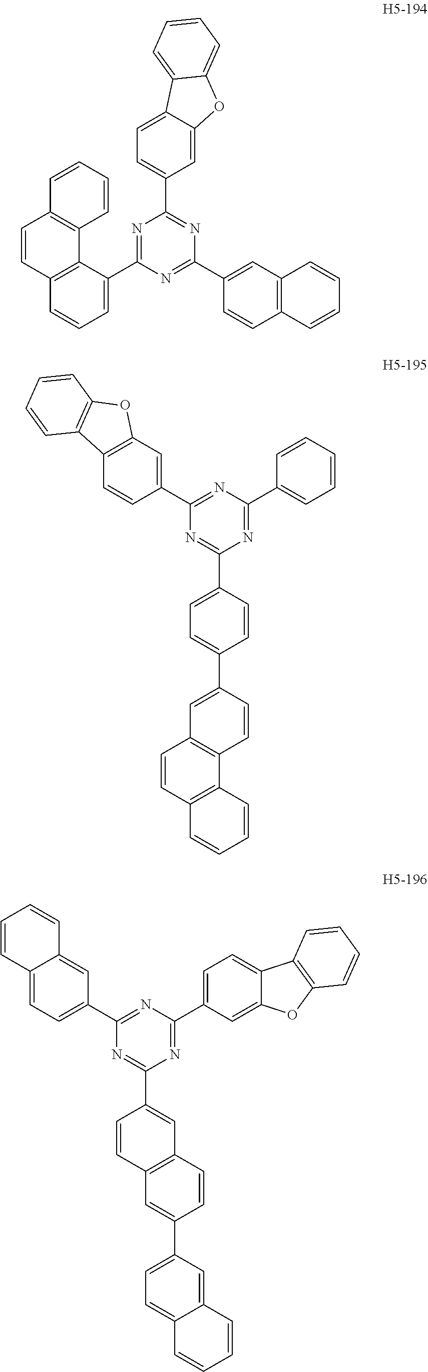

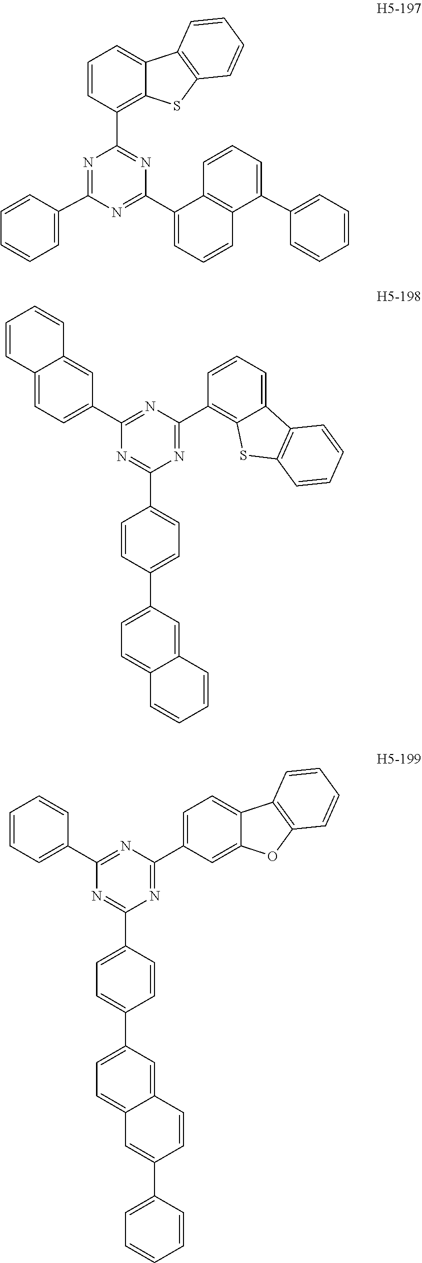

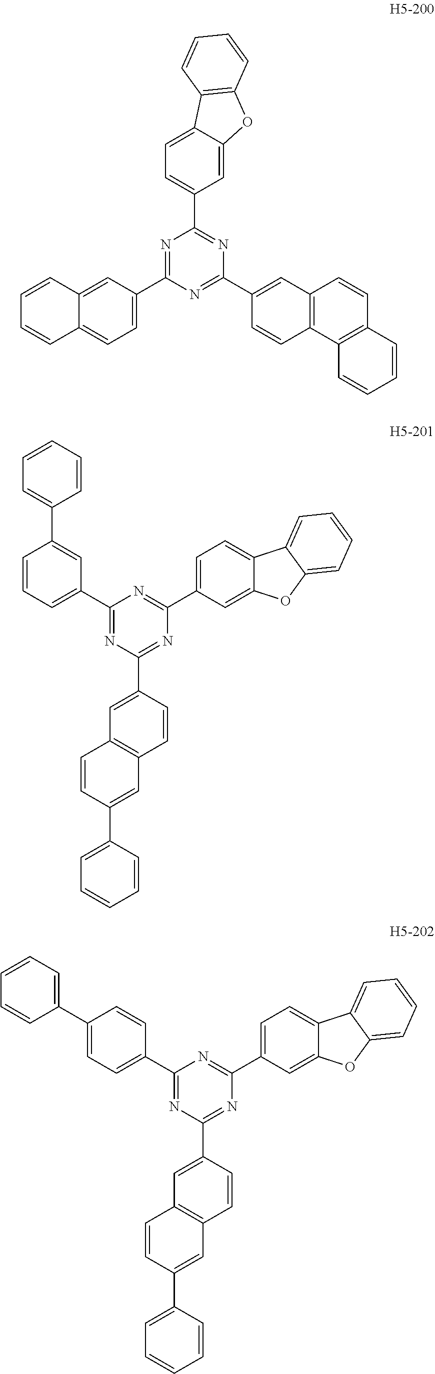 [SMILES: c1ccc2cc(-c3nc(-c4ccc5c(ccc6ccccc65)c4)nc(-c4ccc5c(c4)oc4ccccc45)n3)ccc2c1 ; c1ccc(-c2cccc(-c3nc(-c4ccc5cc(-c6ccccc6)ccc5c4)nc(-c4ccc5c(c4)oc4ccccc45)n3)c2)cc1 ; c1ccc(-c2ccc(-c3nc(-c4ccc5cc(-c6ccccc6)ccc5c4)nc(-c4ccc5c(c4)oc4ccccc45)n3)cc2)cc1]

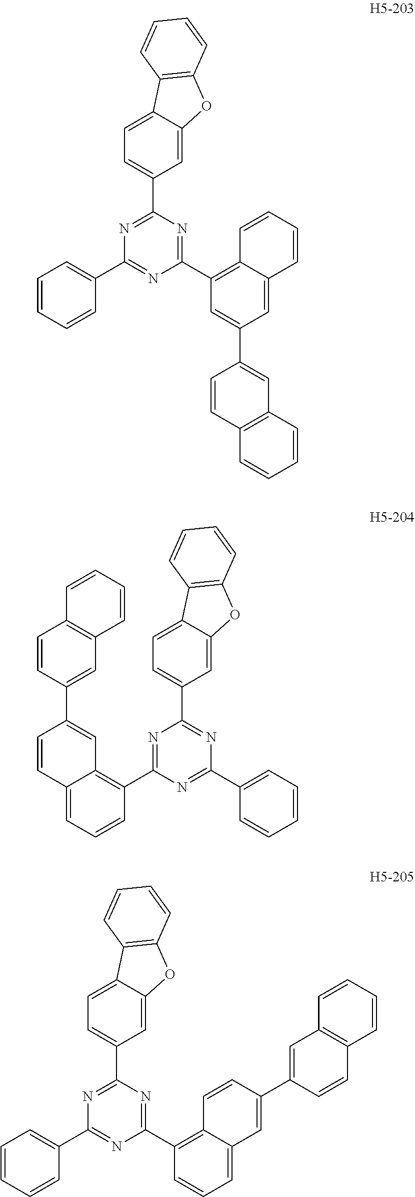 [SMILES: c1ccc(-c2nc(-c3ccc4c(c3)oc3ccccc34)nc(-c3cc(-c4ccc5ccccc5c4)cc4ccccc34)n2)cc1 ; c1ccc(-c2nc(-c3ccc4c(c3)oc3ccccc34)nc(-c3cccc4ccc(-c5ccc6ccccc6c5)cc34)n2)cc1 ; c1ccc(-c2nc(-c3ccc4c(c3)oc3ccccc34)nc(-c3cccc4cc(-c5ccc6ccccc6c5)ccc34)n2)cc1]

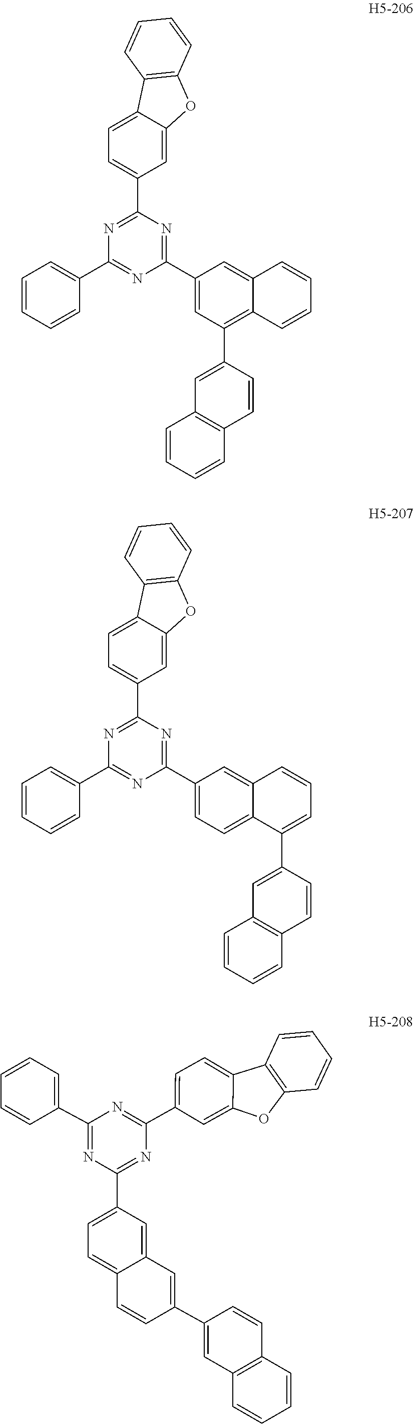 [SMILES: c1ccc(-c2nc(-c3cc(-c4ccc5ccccc5c4)c4ccccc4c3)nc(-c3ccc4c(c3)oc3ccccc34)n2)cc1 ; c1ccc(-c2nc(-c3ccc4c(-c5ccc6ccccc6c5)cccc4c3)nc(-c3ccc4c(c3)oc3ccccc34)n2)cc1 ; c1ccc(-c2nc(-c3ccc4ccc(-c5ccc6ccccc6c5)cc4c3)nc(-c3ccc4c(c3)oc3ccccc34)n2)cc1]

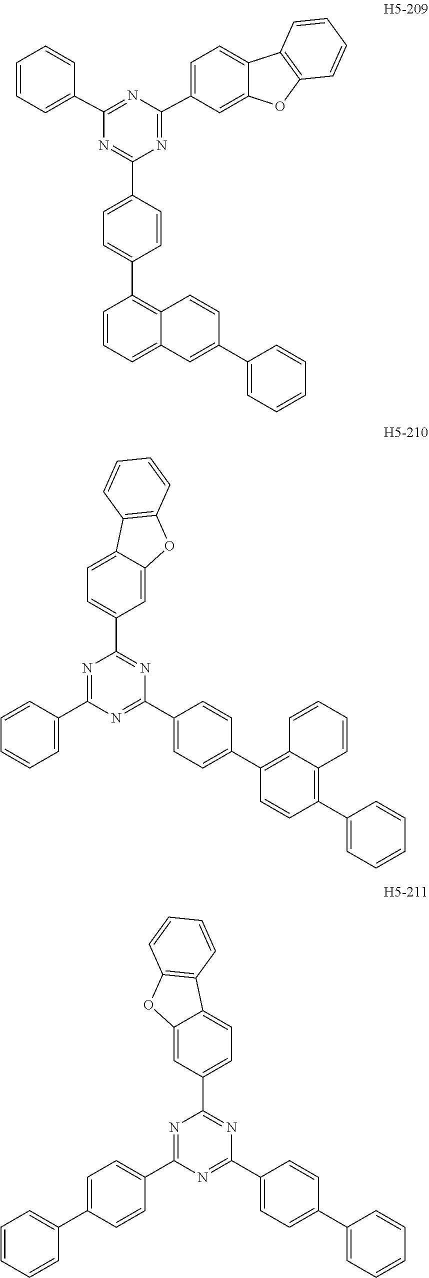 [SMILES: c1ccc(-c2ccc3c(-c4ccc(-c5nc(-c6ccccc6)nc(-c6ccc7c(c6)oc6ccccc67)n5)cc4)cccc3c2)cc1 ; c1ccc(-c2nc(-c3ccc(-c4ccc(-c5ccccc5)c5ccccc45)cc3)nc(-c3ccc4c(c3)oc3ccccc34)n2)cc1 ; c1ccc(-c2ccc(-c3nc(-c4ccc(-c5ccccc5)cc4)nc(-c4ccc5c(c4)oc4ccccc45)n3)cc2)cc1]

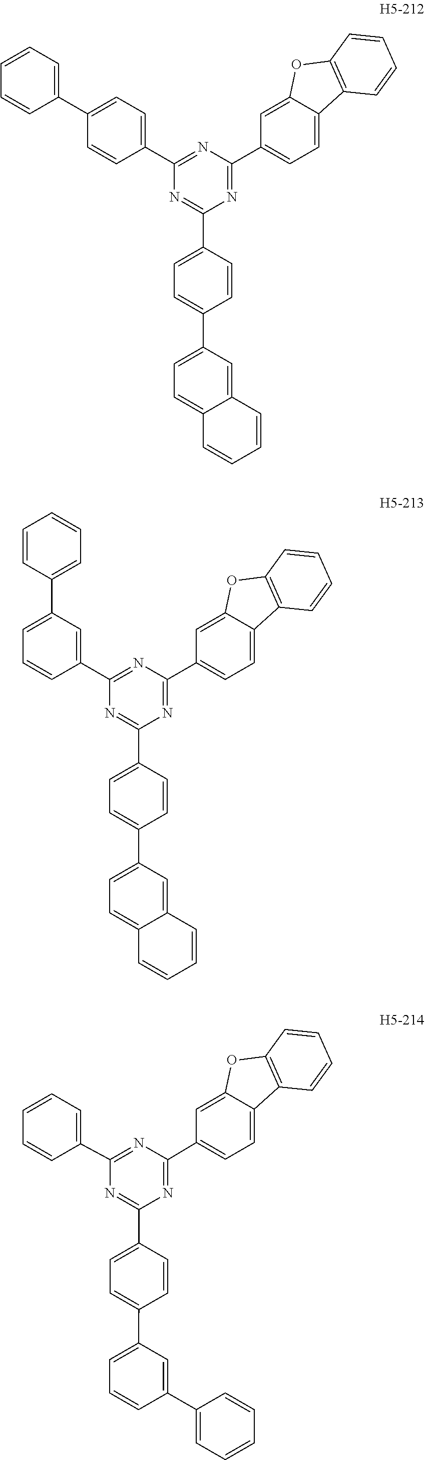 [SMILES: c1ccc(-c2ccc(-c3nc(-c4ccc(-c5ccc6ccccc6c5)cc4)nc(-c4ccc5c(c4)oc4ccccc45)n3)cc2)cc1 ; c1ccc(-c2cccc(-c3nc(-c4ccc(-c5ccc6ccccc6c5)cc4)nc(-c4ccc5c(c4)oc4ccccc45)n3)c2)cc1 ; c1ccc(-c2cccc(-c3ccc(-c4nc(-c5ccccc5)nc(-c5ccc6c(c5)oc5ccccc56)n4)cc3)c2)cc1]

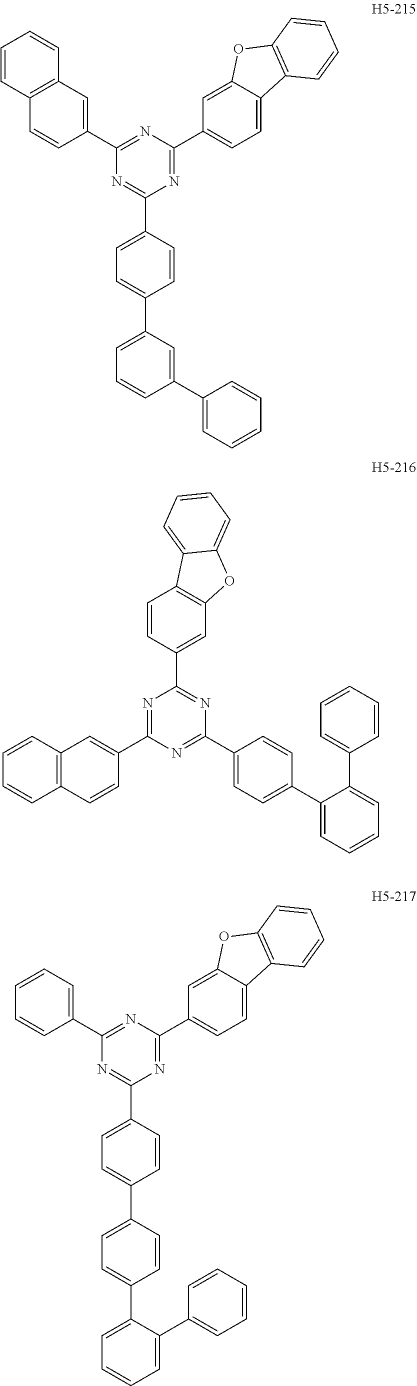 [SMILES: c1ccc(-c2cccc(-c3ccc(-c4nc(-c5ccc6ccccc6c5)nc(-c5ccc6c(c5)oc5ccccc56)n4)cc3)c2)cc1 ; c1ccc(-c2ccccc2-c2ccc(-c3nc(-c4ccc5ccccc5c4)nc(-c4ccc5c(c4)oc4ccccc45)n3)cc2)cc1 ; c1ccc(-c2nc(-c3ccc(-c4ccc(-c5ccccc5-c5ccccc5)cc4)cc3)nc(-c3ccc4c(c3)oc3ccccc34)n2)cc1]

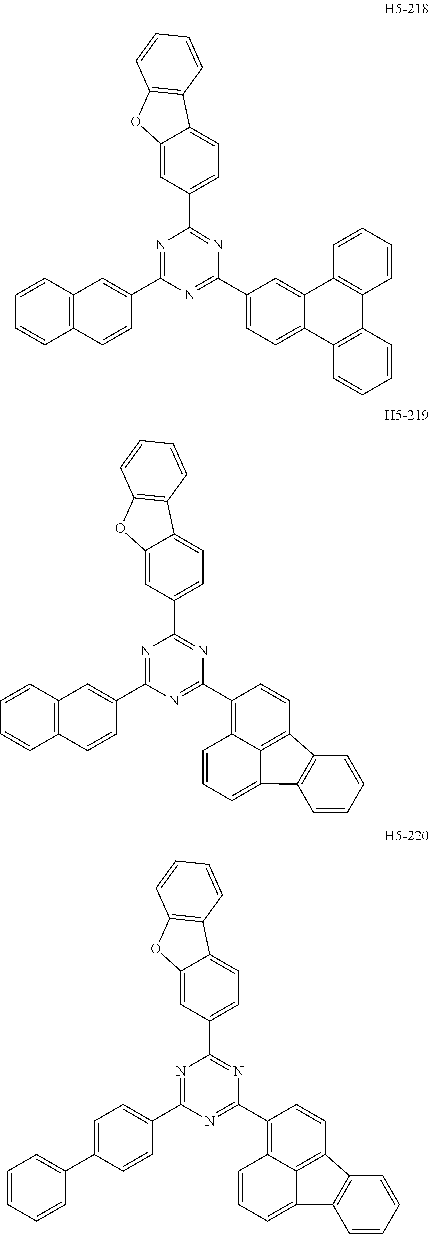 [SMILES: c1ccc2cc(-c3nc(-c4ccc5c(c4)oc4ccccc45)nc(-c4ccc5c6ccccc6c6ccccc6c5c4)n3)ccc2c1 ; c1ccc2c(c1)-c1cccc3c(-c4nc(-c5ccc6ccccc6c5)nc(-c5ccc6c(c5)oc5ccccc56)n4)ccc-2c13 ; c1ccc(-c2ccc(-c3nc(-c4ccc5c(c4)oc4ccccc45)nc(-c4ccc5c6c(cccc46)-c4ccccc4-5)n3)cc2)cc1]

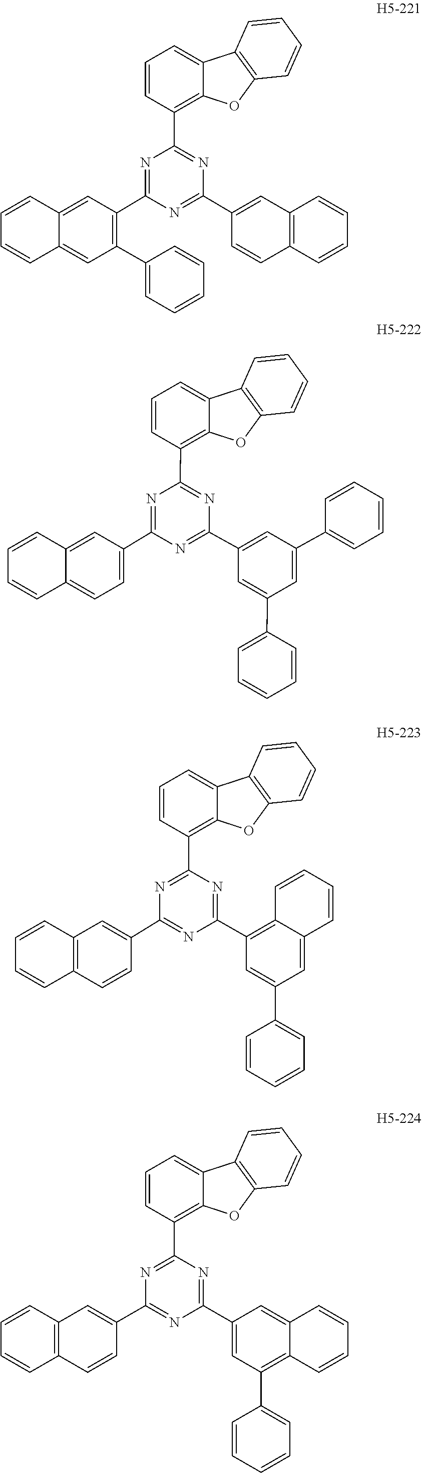

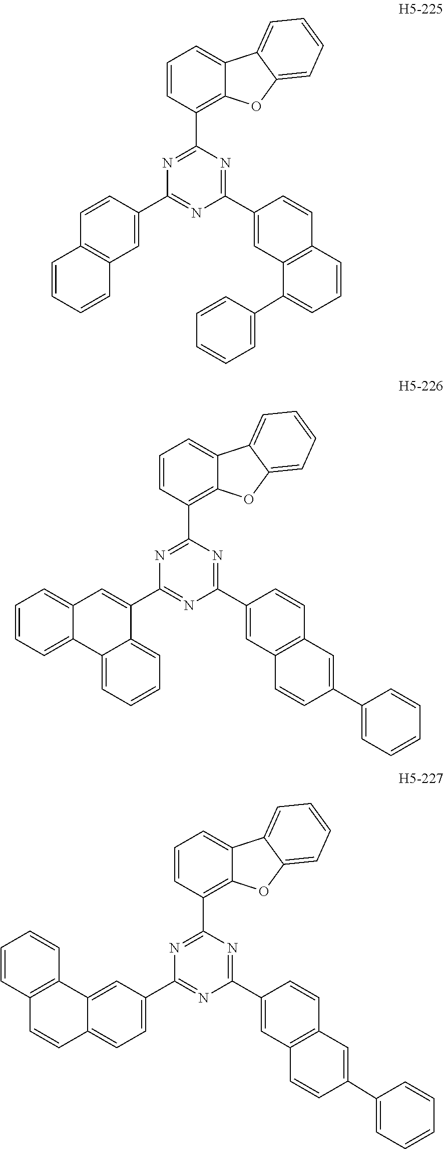 [SMILES: c1ccc(-c2cccc3ccc(-c4nc(-c5ccc6ccccc6c5)nc(-c5cccc6c5oc5ccccc56)n4)cc23)cc1 ; c1ccc(-c2ccc3cc(-c4nc(-c5cc6ccccc6c6ccccc56)nc(-c5cccc6c5oc5ccccc56)n4)ccc3c2)cc1 ; c1ccc(-c2ccc3cc(-c4nc(-c5ccc6ccc7ccccc7c6c5)nc(-c5cccc6c5oc5ccccc56)n4)ccc3c2)cc1]

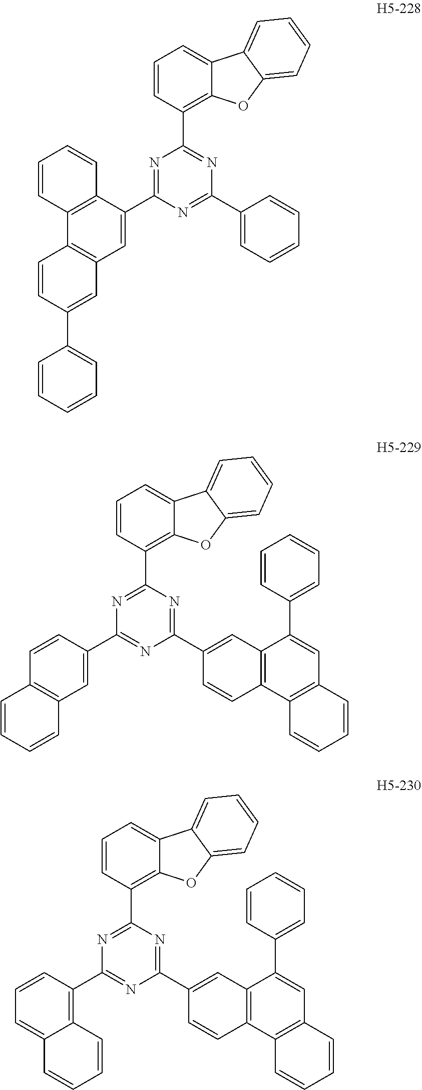 [SMILES: c1ccc(-c2ccc3c(c2)cc(-c2nc(-c4ccccc4)nc(-c4cccc5c4oc4ccccc45)n2)c2ccccc23)cc1 ; c1ccc(-c2cc3ccccc3c3ccc(-c4nc(-c5ccc6ccccc6c5)nc(-c5cccc6c5oc5ccccc56)n4)cc23)cc1 ; c1ccc(-c2cc3ccccc3c3ccc(-c4nc(-c5cccc6ccccc56)nc(-c5cccc6c5oc5ccccc56)n4)cc23)cc1]

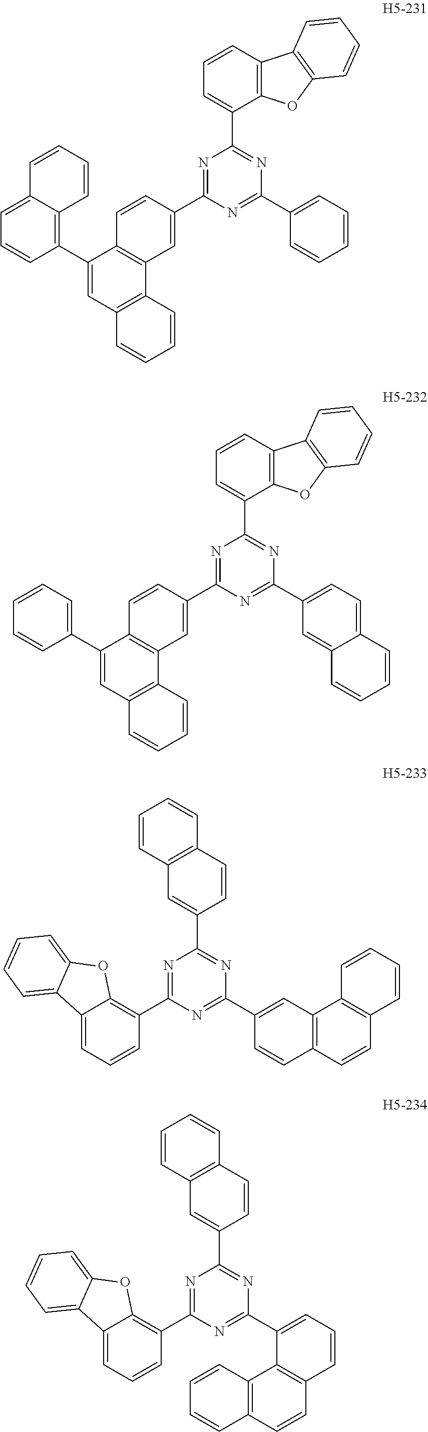 [SMILES: c1ccc(-c2nc(-c3ccc4c(-c5cccc6ccccc56)cc5ccccc5c4c3)nc(-c3cccc4c3oc3ccccc34)n2)cc1 ; c1ccc(-c2cc3ccccc3c3cc(-c4nc(-c5ccc6ccccc6c5)nc(-c5cccc6c5oc5ccccc56)n4)ccc23)cc1 ; c1ccc2cc(-c3nc(-c4ccc5ccc6ccccc6c5c4)nc(-c4cccc5c4oc4ccccc45)n3)ccc2c1 ; c1ccc2cc(-c3nc(-c4cccc5c4oc4ccccc45)nc(-c4cccc5ccc6ccccc6c45)n3)ccc2c1]

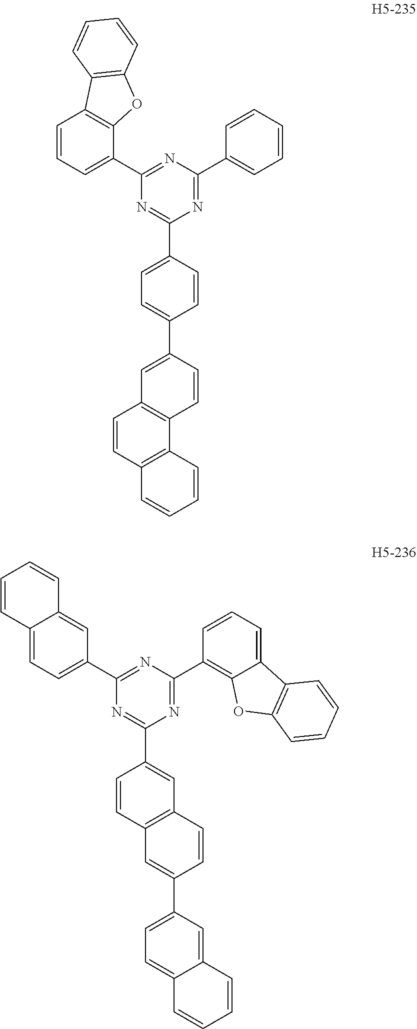 [SMILES: c1ccc(-c2nc(-c3ccc(-c4ccc5c(ccc6ccccc65)c4)cc3)nc(-c3cccc4c3oc3ccccc34)n2)cc1 ; c1ccc2cc(-c3ccc4cc(-c5nc(-c6ccc7ccccc7c6)nc(-c6cccc7c6oc6ccccc67)n5)ccc4c3)ccc2c1]

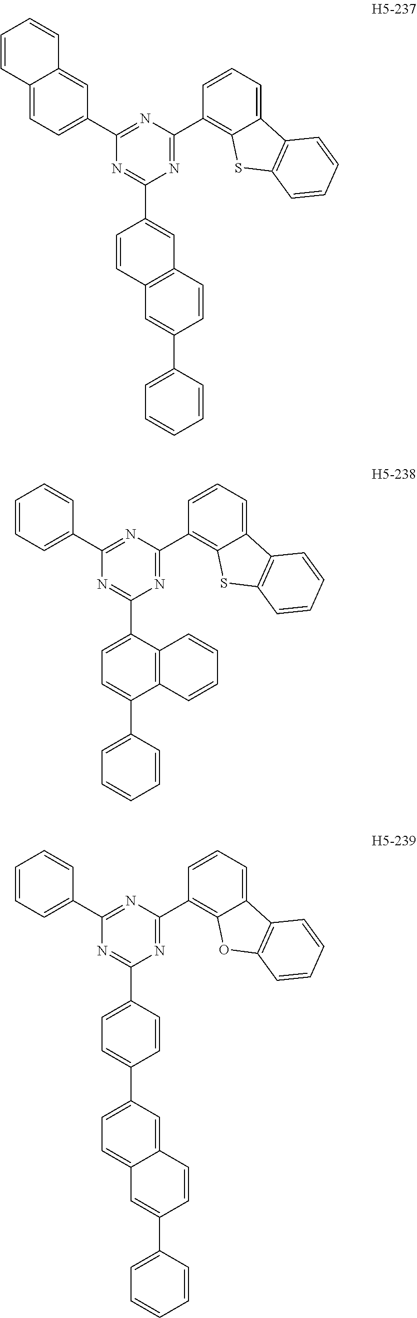 [SMILES: c1ccc(-c2ccc3cc(-c4nc(-c5ccc6ccccc6c5)nc(-c5cccc6c5sc5ccccc56)n4)ccc3c2)cc1 ; c1ccc(-c2nc(-c3ccc(-c4ccccc4)c4ccccc34)nc(-c3cccc4c3sc3ccccc34)n2)cc1 ; c1ccc(-c2ccc3cc(-c4ccc(-c5nc(-c6ccccc6)nc(-c6cccc7c6oc6ccccc67)n5)cc4)ccc3c2)cc1]

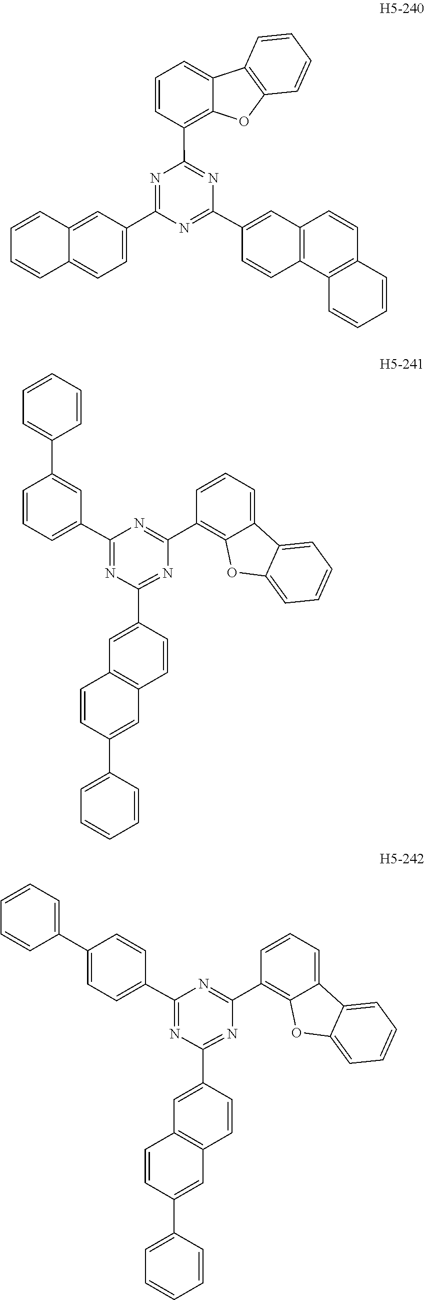 [SMILES: c1ccc2cc(-c3nc(-c4ccc5c(ccc6ccccc65)c4)nc(-c4cccc5c4oc4ccccc45)n3)ccc2c1 ; c1ccc(-c2cccc(-c3nc(-c4ccc5cc(-c6ccccc6)ccc5c4)nc(-c4cccc5c4oc4ccccc45)n3)c2)cc1 ; c1ccc(-c2ccc(-c3nc(-c4ccc5cc(-c6ccccc6)ccc5c4)nc(-c4cccc5c4oc4ccccc45)n3)cc2)cc1]

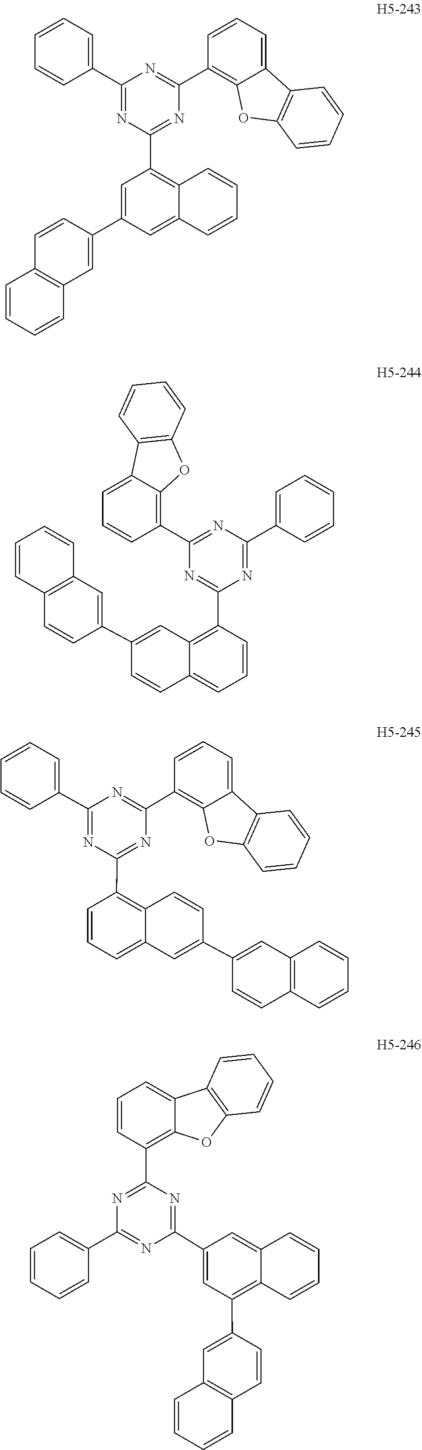 [SMILES: c1ccc(-c2nc(-c3cc(-c4ccc5ccccc5c4)cc4ccccc34)nc(-c3cccc4c3oc3ccccc34)n2)cc1 ; c1ccc(-c2nc(-c3cccc4ccc(-c5ccc6ccccc6c5)cc34)nc(-c3cccc4c3oc3ccccc34)n2)cc1 ; c1ccc(-c2nc(-c3cccc4cc(-c5ccc6ccccc6c5)ccc34)nc(-c3cccc4c3oc3ccccc34)n2)cc1 ; c1ccc(-c2nc(-c3cc(-c4ccc5ccccc5c4)c4ccccc4c3)nc(-c3cccc4c3oc3ccccc34)n2)cc1]

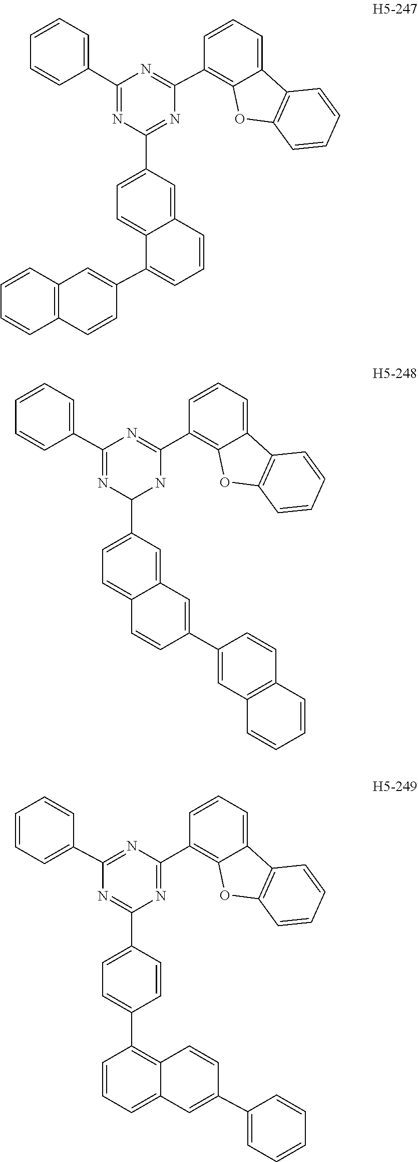

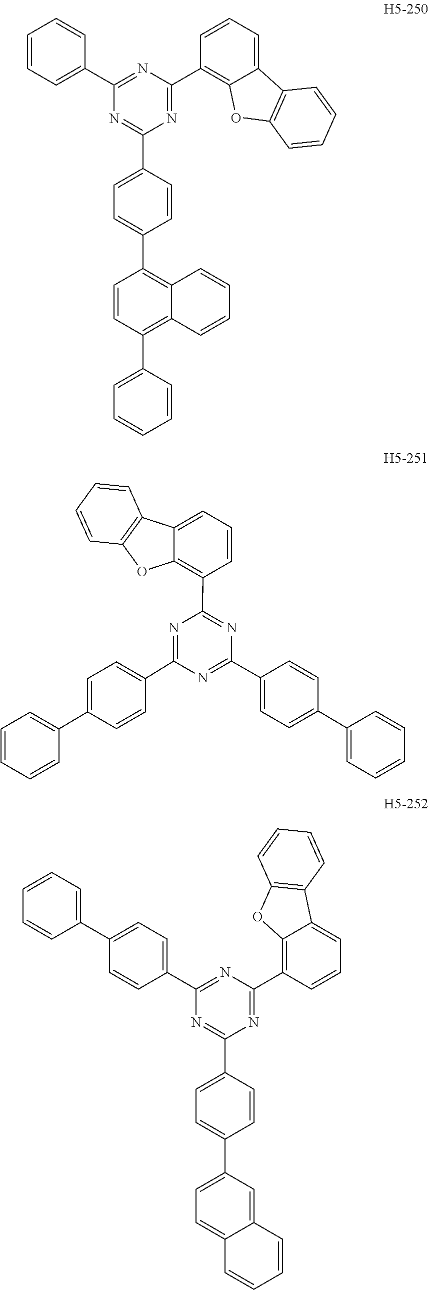

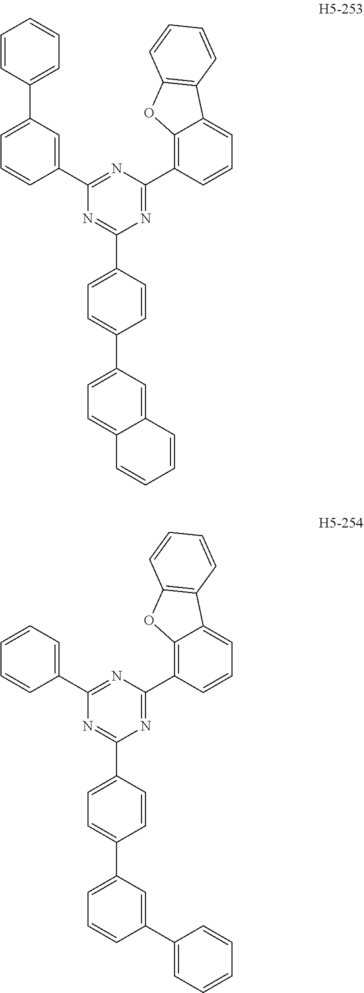 [SMILES: c1ccc(-c2cccc(-c3nc(-c4ccc(-c5ccc6ccccc6c5)cc4)nc(-c4cccc5c4oc4ccccc45)n3)c2)cc1 ; c1ccc(-c2cccc(-c3ccc(-c4nc(-c5ccccc5)nc(-c5cccc6c5oc5ccccc56)n4)cc3)c2)cc1]

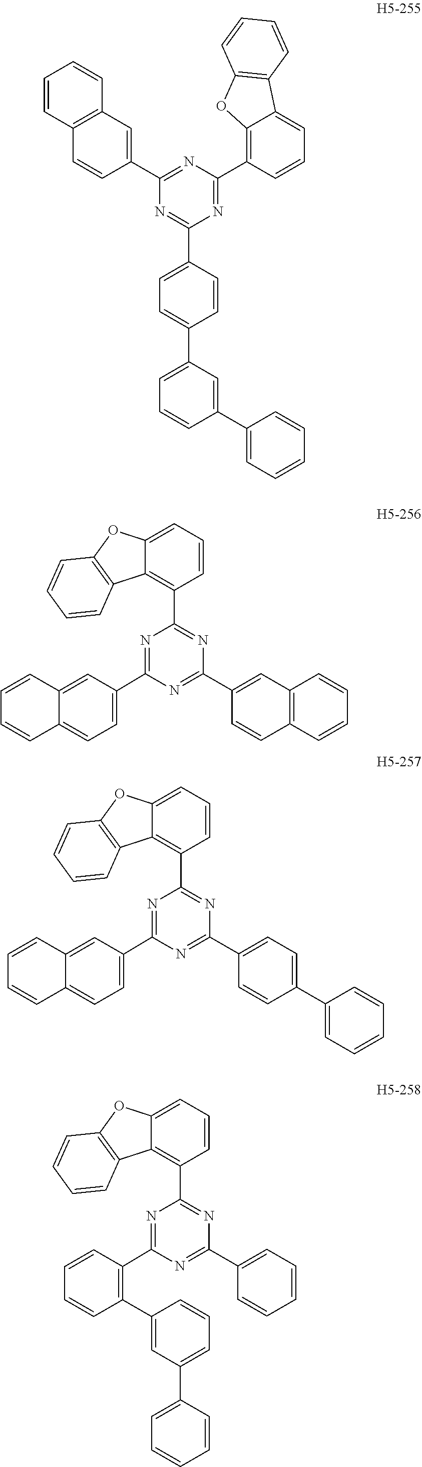 [SMILES: c1ccc(-c2cccc(-c3ccc(-c4nc(-c5ccc6ccccc6c5)nc(-c5cccc6c5oc5ccccc56)n4)cc3)c2)cc1 ; c1ccc2cc(-c3nc(-c4ccc5ccccc5c4)nc(-c4cccc5oc6ccccc6c45)n3)ccc2c1 ; c1ccc(-c2ccc(-c3nc(-c4ccc5ccccc5c4)nc(-c4cccc5oc6ccccc6c45)n3)cc2)cc1 ; c1ccc(-c2cccc(-c3ccccc3-c3nc(-c4ccccc4)nc(-c4cccc5oc6ccccc6c45)n3)c2)cc1]

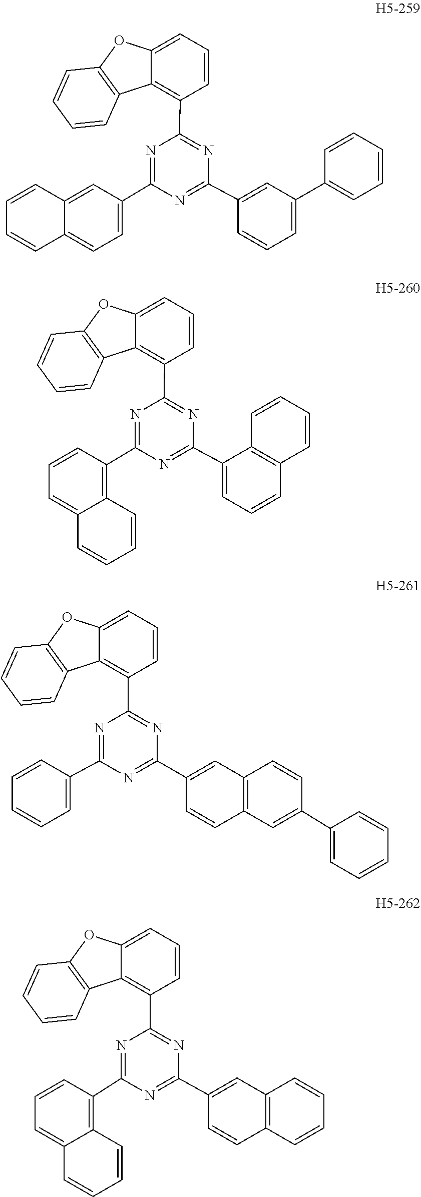 [SMILES: c1ccc(-c2cccc(-c3nc(-c4ccc5ccccc5c4)nc(-c4cccc5oc6ccccc6c45)n3)c2)cc1 ; c1ccc2c(-c3nc(-c4cccc5ccccc45)nc(-c4cccc5oc6ccccc6c45)n3)cccc2c1 ; c1ccc(-c2ccc3cc(-c4nc(-c5ccccc5)nc(-c5cccc6oc7ccccc7c56)n4)ccc3c2)cc1 ; c1ccc2cc(-c3nc(-c4cccc5ccccc45)nc(-c4cccc5oc6ccccc6c45)n3)ccc2c1]

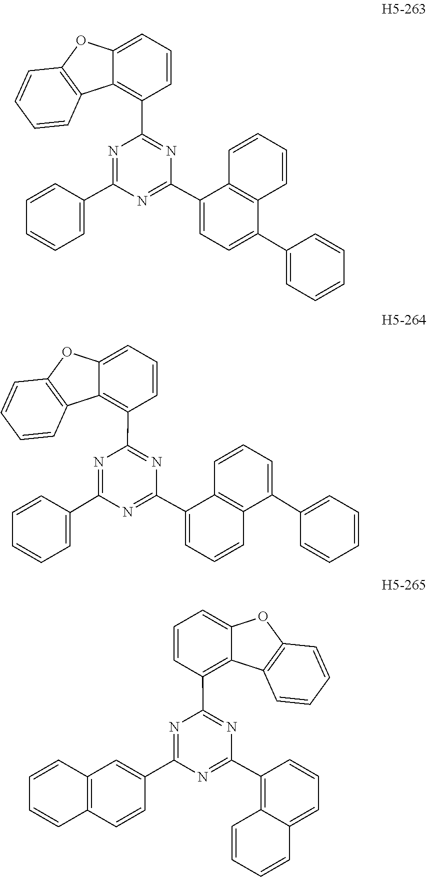 [SMILES: c1ccc(-c2nc(-c3ccc(-c4ccccc4)c4ccccc34)nc(-c3cccc4oc5ccccc5c34)n2)cc1 ; c1ccc(-c2nc(-c3cccc4c(-c5ccccc5)cccc34)nc(-c3cccc4oc5ccccc5c34)n2)cc1 ; c1ccc2cc(-c3nc(-c4cccc5ccccc45)nc(-c4cccc5oc6ccccc6c45)n3)ccc2c1]

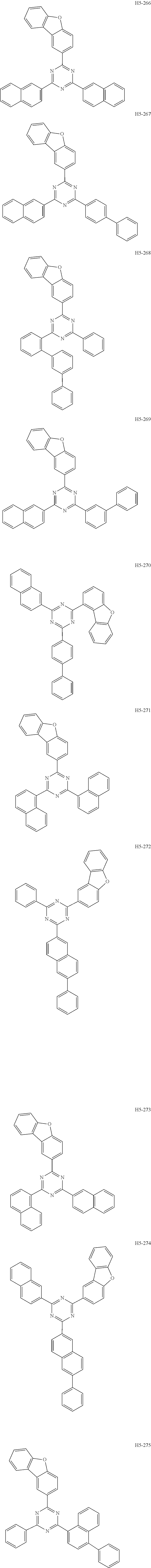

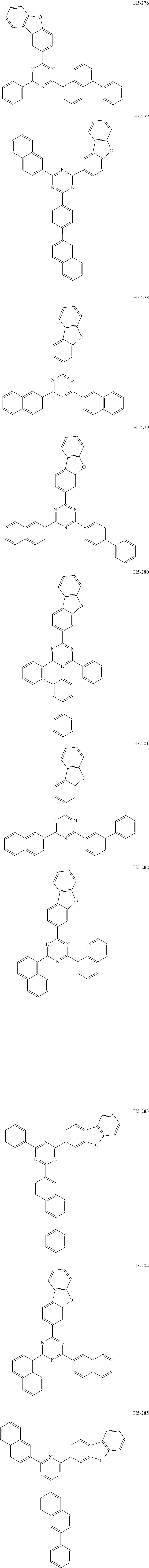

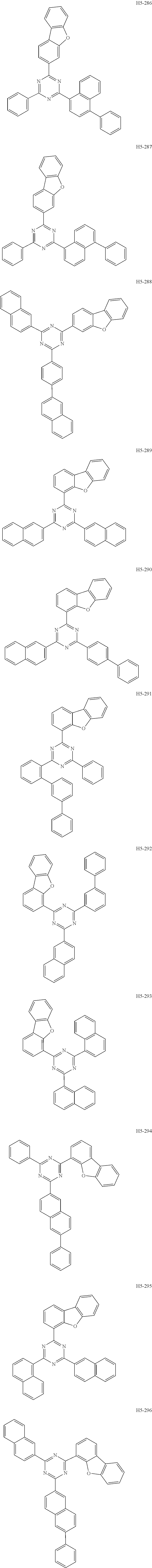

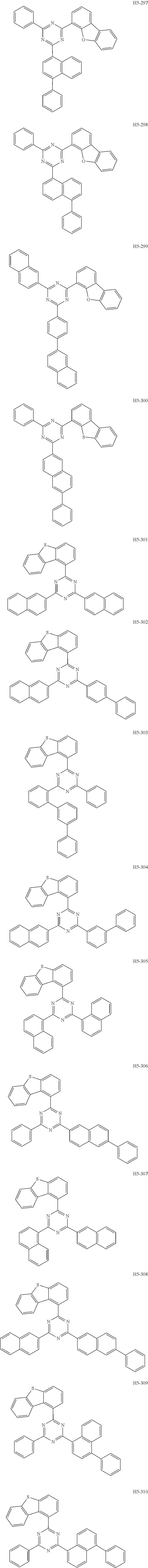

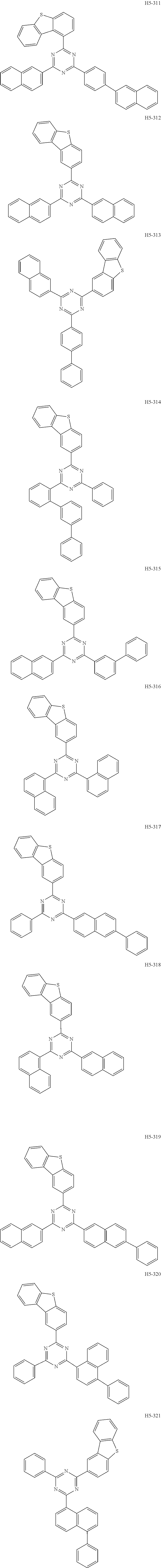

[chemical structure]

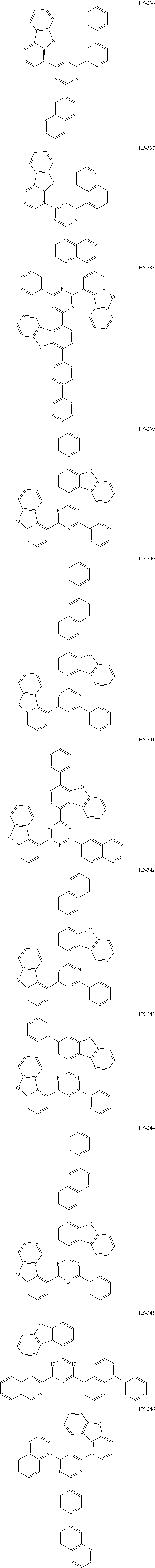

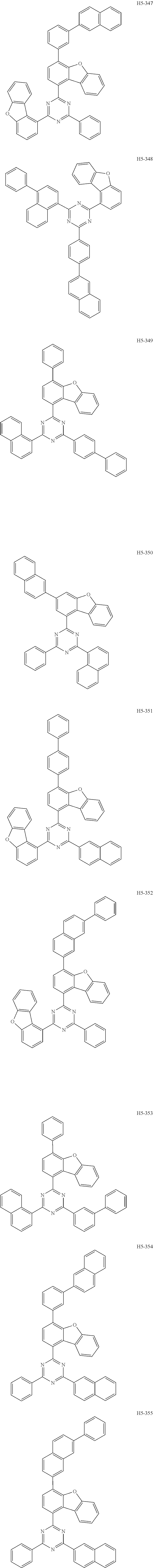

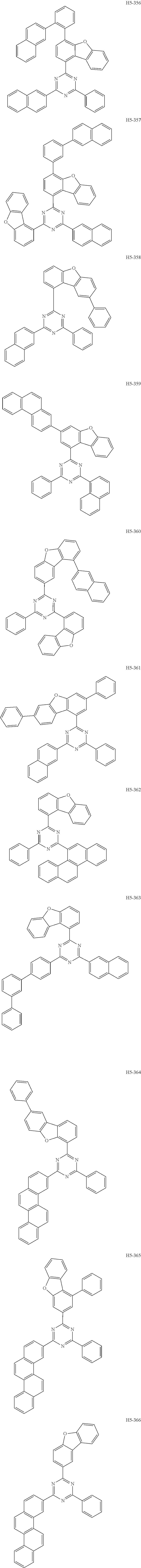

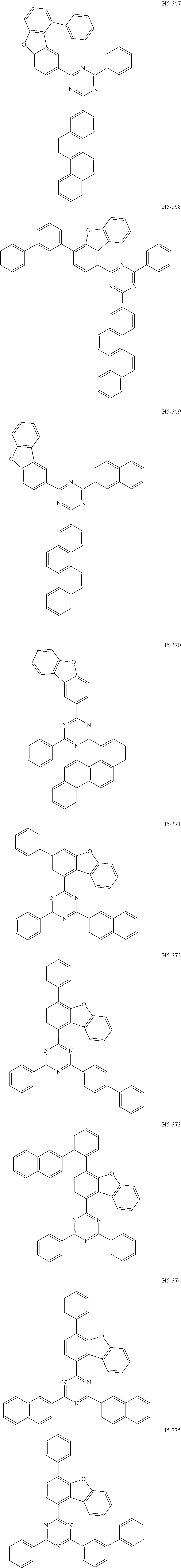

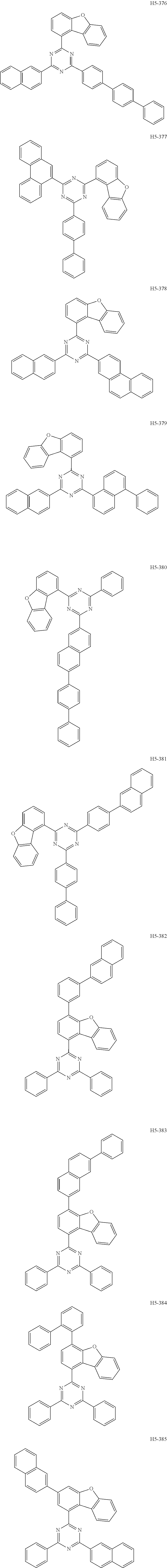

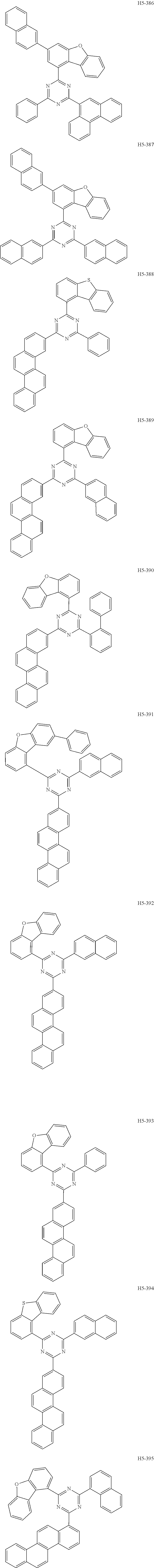

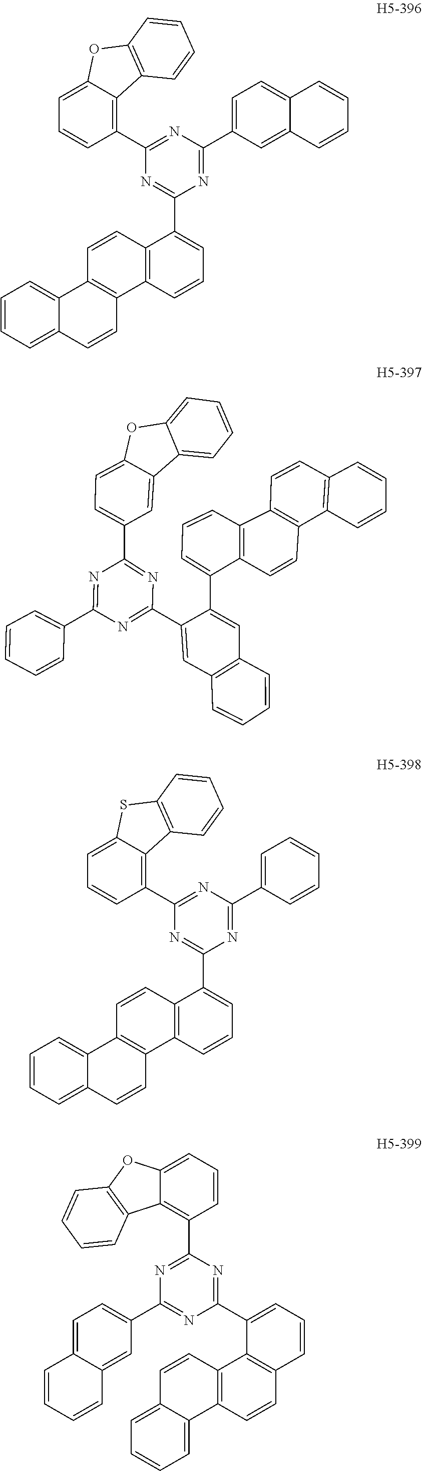 [SMILES: c1ccc2cc(-c3nc(-c4cccc5c4ccc4c6ccccc6ccc54)nc(-c4cccc5oc6ccccc6c45)n3)ccc2c1 ; c1ccc(-c2nc(-c3ccc4oc5ccccc5c4c3)nc(-c3cc4ccccc4cc3-c3cccc4c3ccc3c5ccccc5ccc43)n2)cc1 ; c1ccc(-c2nc(-c3cccc4c3ccc3c5ccccc5ccc43)nc(-c3cccc4sc5ccccc5c34)n2)cc1 ; c1ccc2cc(-c3nc(-c4cccc5ccc6c7ccccc7ccc6c45)nc(-c4cccc5oc6ccccc6c45)n3)ccc2c1]

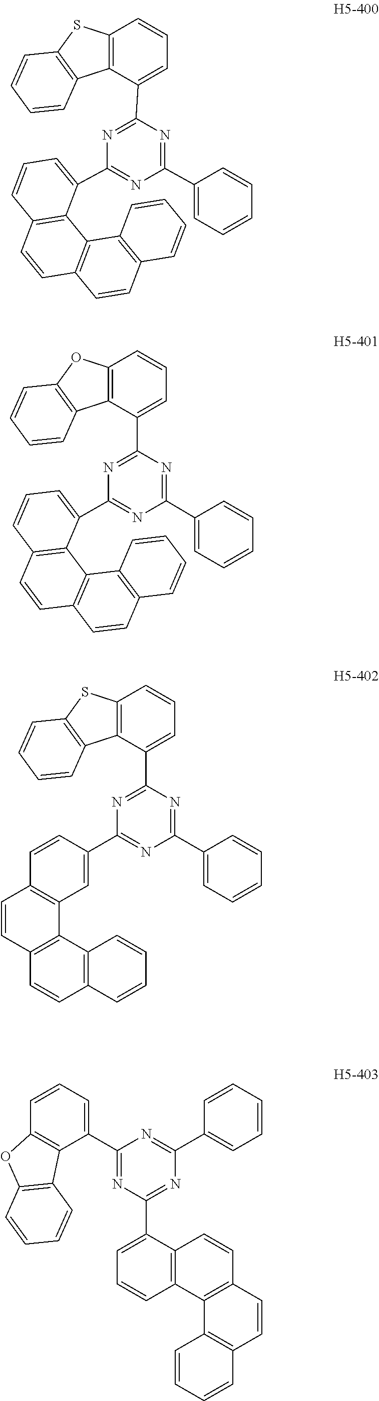 [SMILES: c1ccc(-c2nc(-c3cccc4sc5ccccc5c34)nc(-c3cccc4ccc5ccc6ccccc6c5c34)n2)cc1 ; c1ccc(-c2nc(-c3cccc4oc5ccccc5c34)nc(-c3cccc4ccc5ccc6ccccc6c5c34)n2)cc1 ; c1ccc(-c2nc(-c3ccc4ccc5ccc6ccccc6c5c4c3)nc(-c3cccc4sc5ccccc5c34)n2)cc1 ; c1ccc(-c2nc(-c3cccc4c3ccc3ccc5ccccc5c34)nc(-c3cccc4oc5ccccc5c34)n2)cc1]

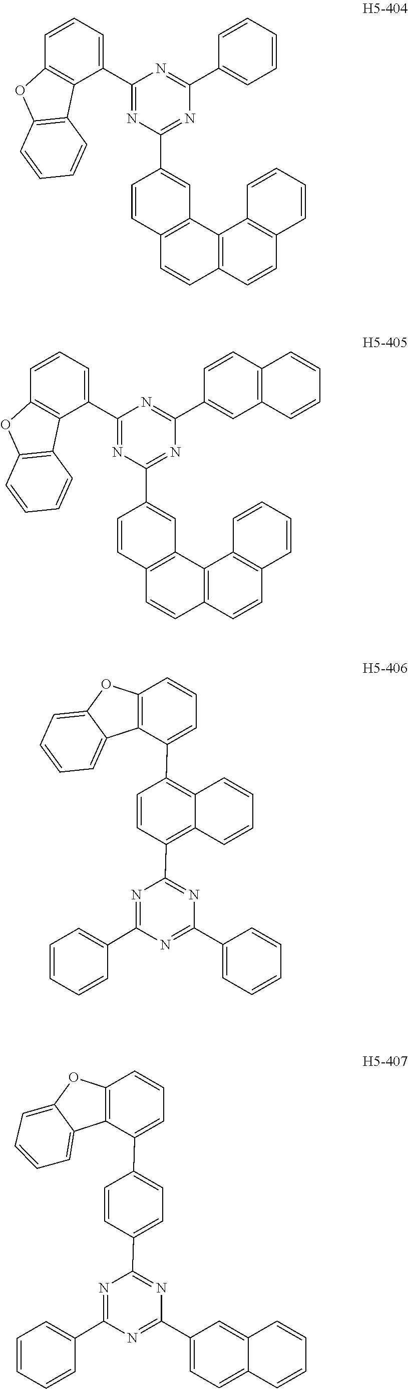 [SMILES: c1ccc(-c2nc(-c3ccc4ccc5ccc6ccccc6c5c4c3)nc(-c3cccc4oc5ccccc5c34)n2)cc1 ; c1ccc2cc(-c3nc(-c4ccc5ccc6ccc7ccccc7c6c5c4)nc(-c4cccc5oc6ccccc6c45)n3)ccc2c1 ; c1ccc(-c2nc(-c3ccccc3)nc(-c3ccc(-c4cccc5oc6ccccc6c45)c4ccccc34)n2)cc1 ; c1ccc(-c2nc(-c3ccc(-c4cccc5oc6ccccc6c45)cc3)nc(-c3ccc4ccccc4c3)n2)cc1]

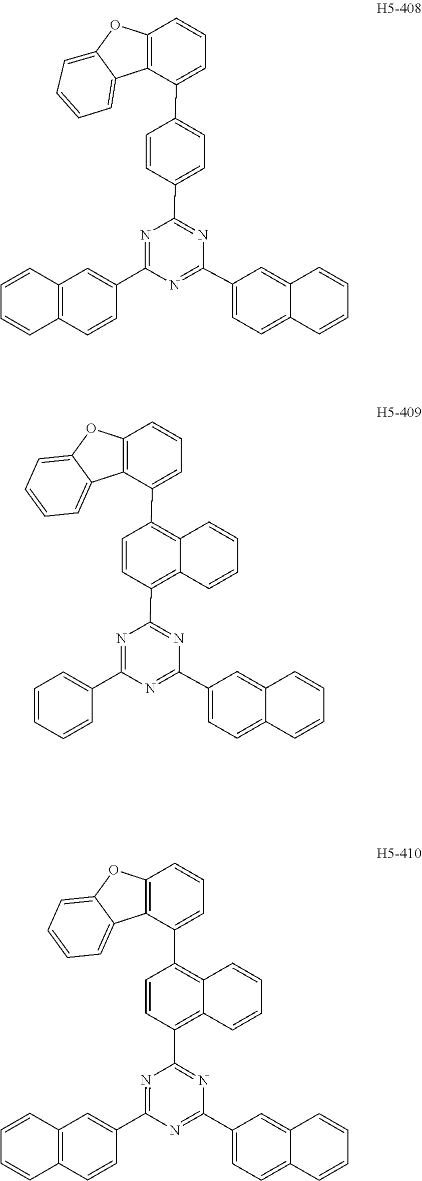

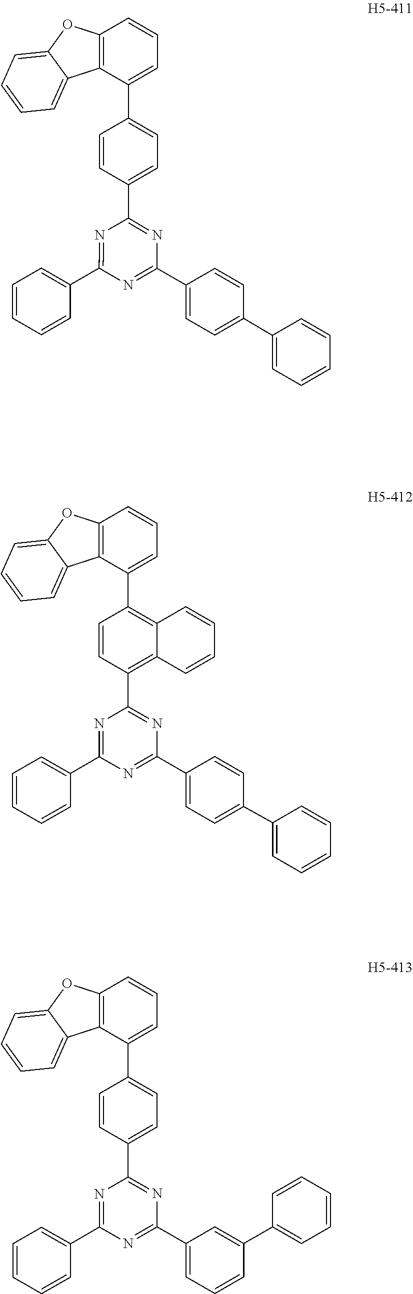

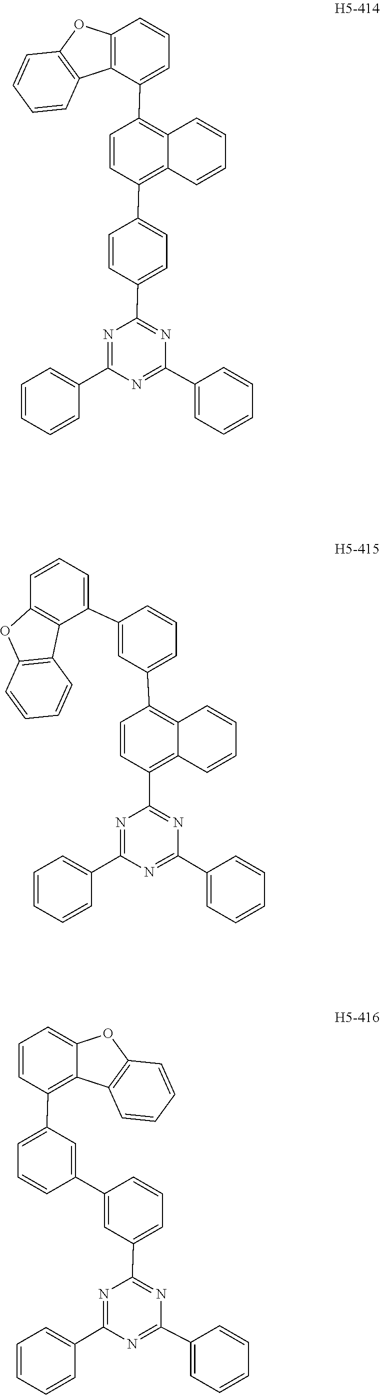

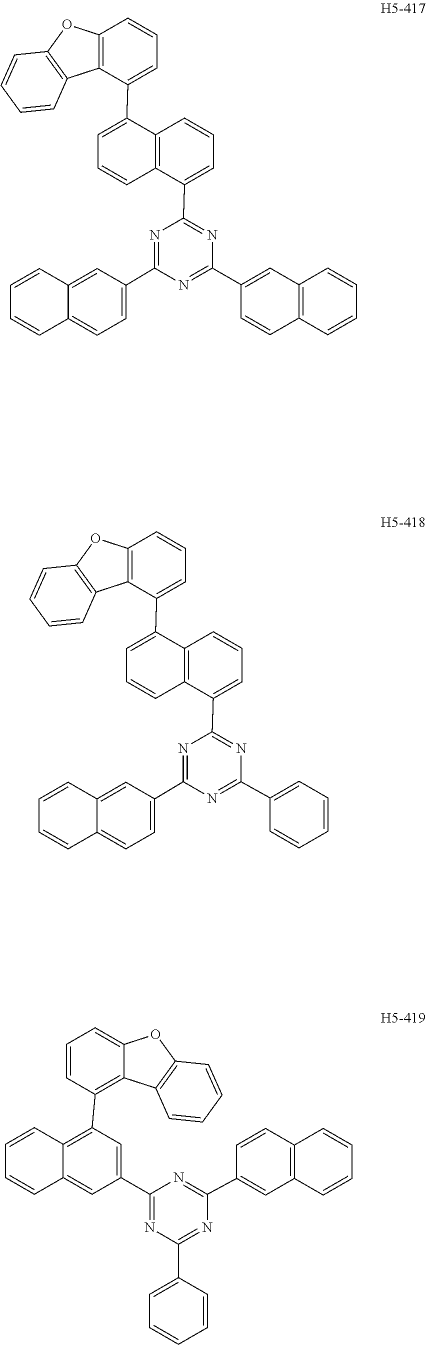

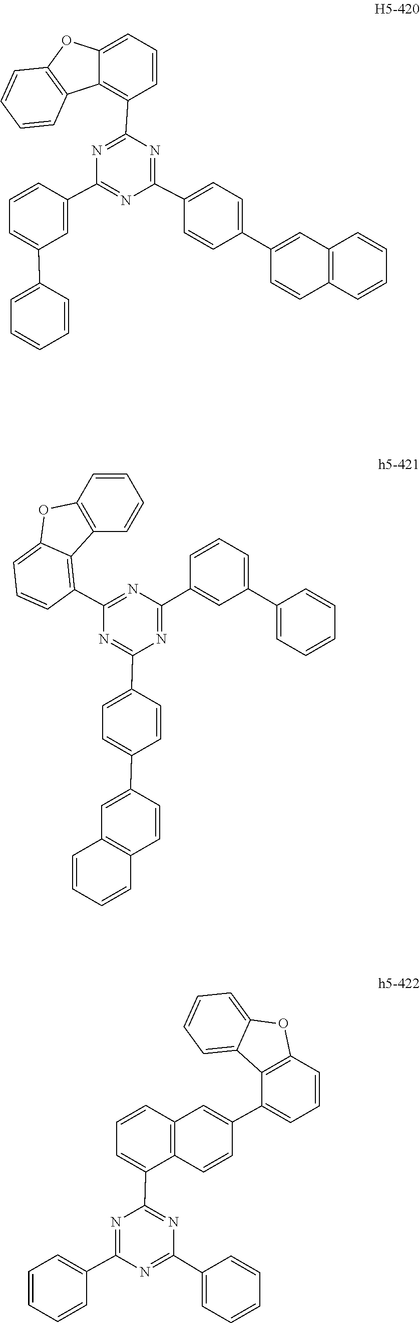 [SMILES: c1ccc(-c2cccc(-c3nc(-c4ccc(-c5ccc6ccccc6c5)cc4)nc(-c4cccc5oc6ccccc6c45)n3)c2)cc1 ; c1ccc(-c2cccc(-c3nc(-c4ccc(-c5ccc6ccccc6c5)cc4)nc(-c4cccc5oc6ccccc6c45)n3)c2)cc1 ; c1ccc(-c2nc(-c3ccccc3)nc(-c3cccc4cc(-c5cccc6oc7ccccc7c56)ccc34)n2)cc1]

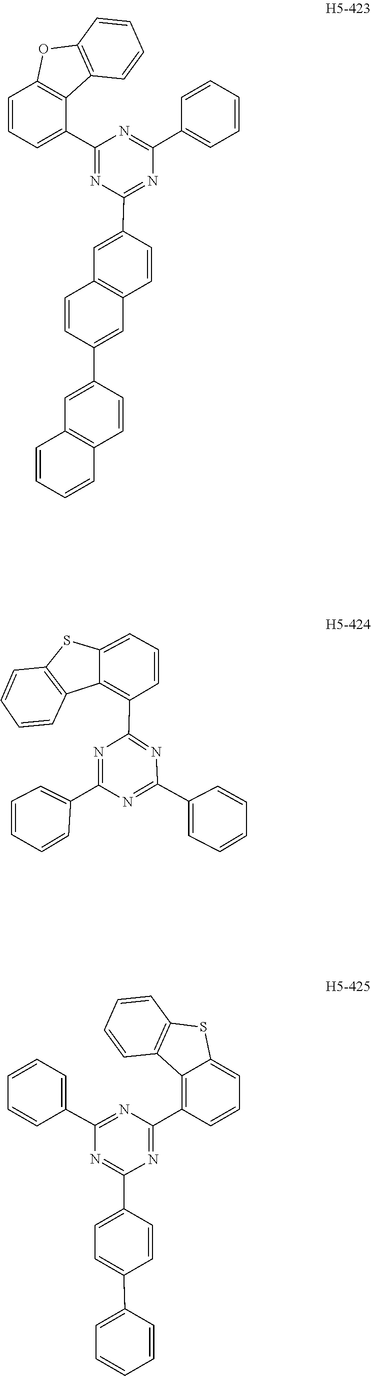

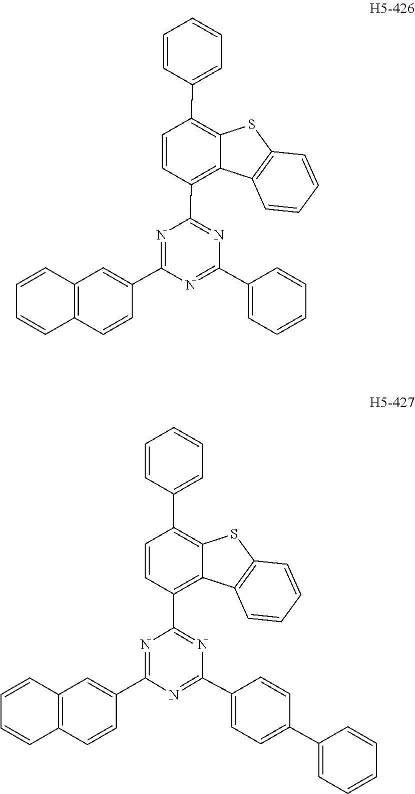

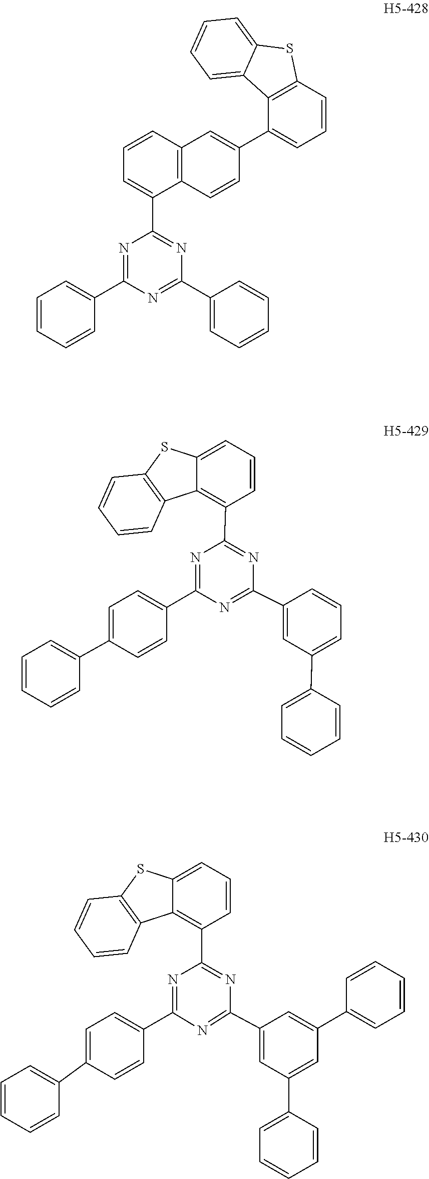

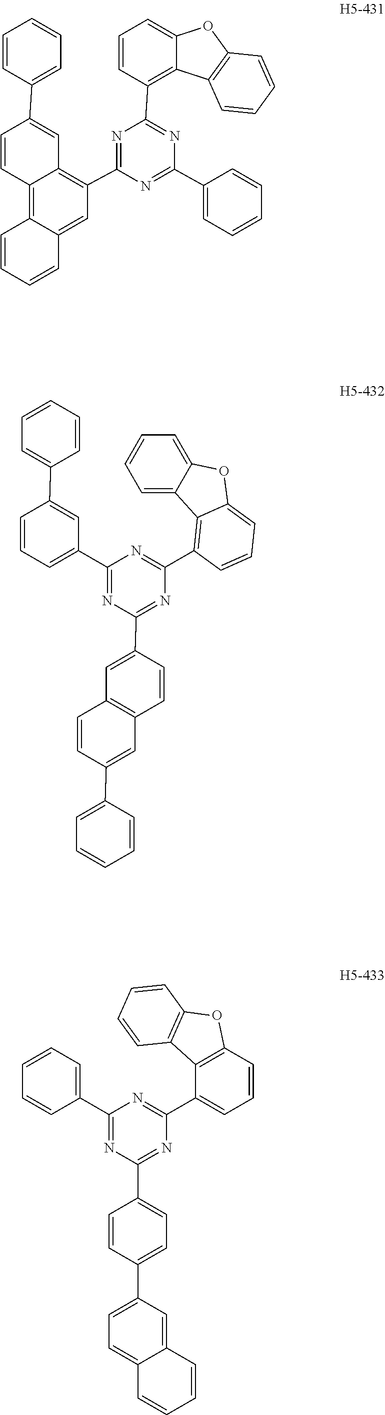 [SMILES: c1ccc(-c2ccc3c(c2)c(-c2nc(-c4ccccc4)nc(-c4cccc5oc6ccccc6c45)n2)cc2ccccc23)cc1 ; c1ccc(-c2cccc(-c3nc(-c4ccc5cc(-c6ccccc6)ccc5c4)nc(-c4cccc5oc6ccccc6c45)n3)c2)cc1 ; c1ccc(-c2nc(-c3ccc(-c4ccc5ccccc5c4)cc3)nc(-c3cccc4oc5ccccc5c34)n2)cc1]

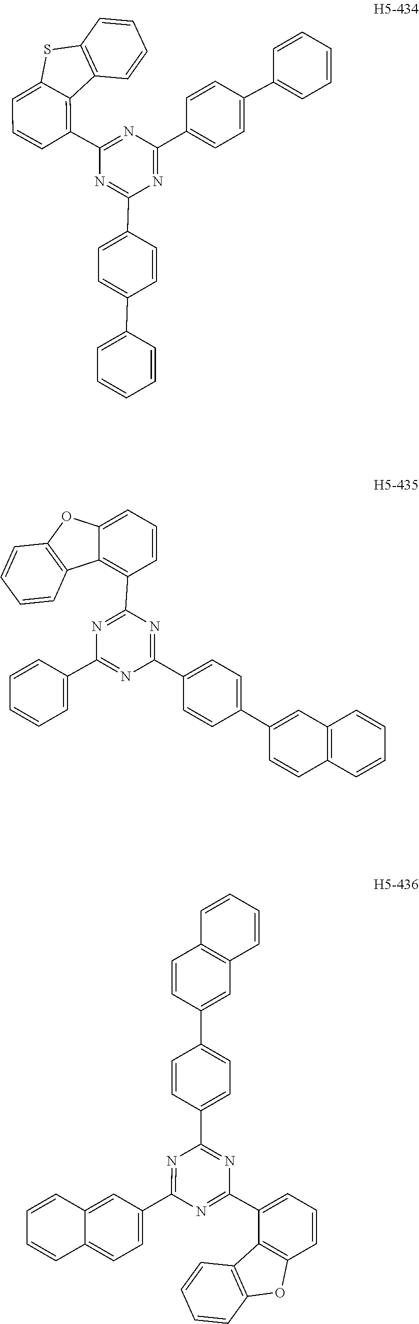

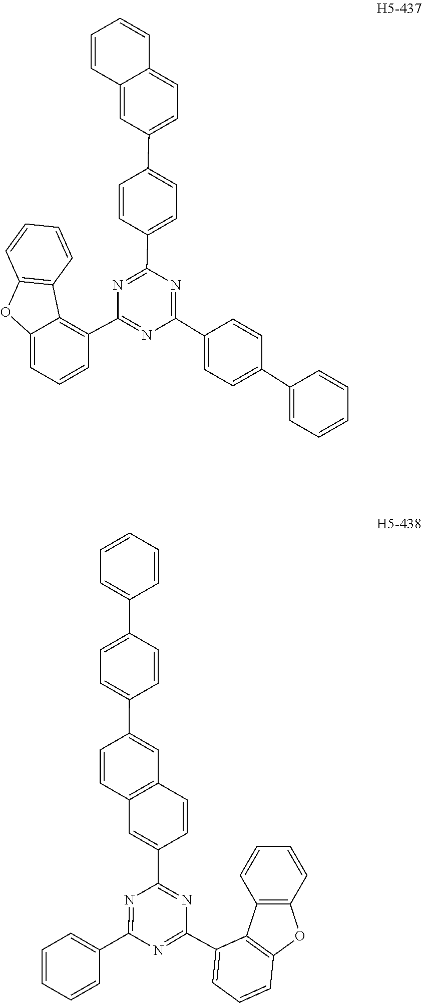

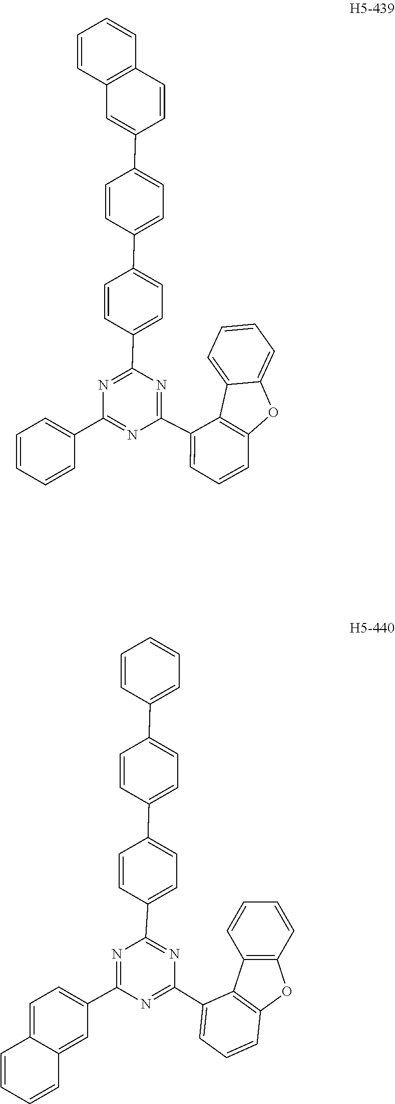 [SMILES: c1ccc(-c2nc(-c3ccc(-c4ccc(-c5ccc6ccccc6c5)cc4)cc3)nc(-c3cccc4oc5ccccc5c34)n2)cc1 ; c1ccc(-c2ccc(-c3ccc(-c4nc(-c5ccc6ccccc6c5)nc(-c5cccc6oc7ccccc7c56)n4)cc3)cc2)cc1]

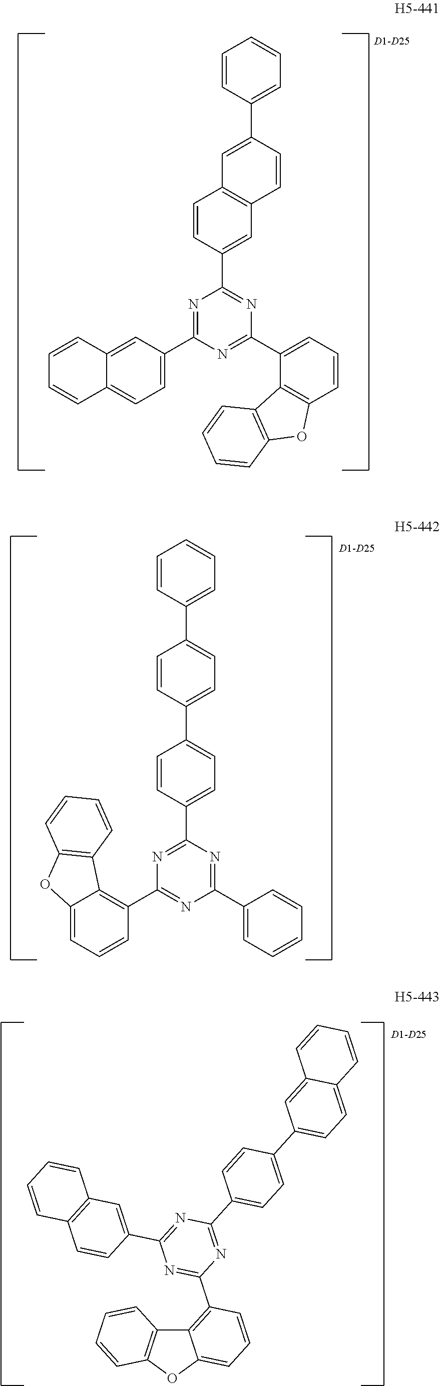

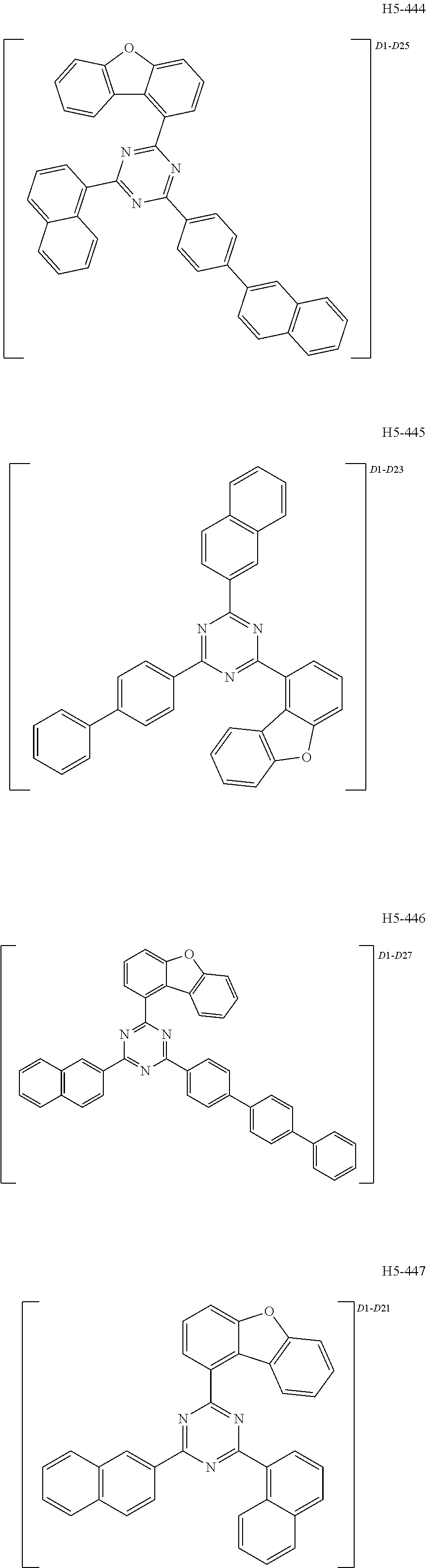

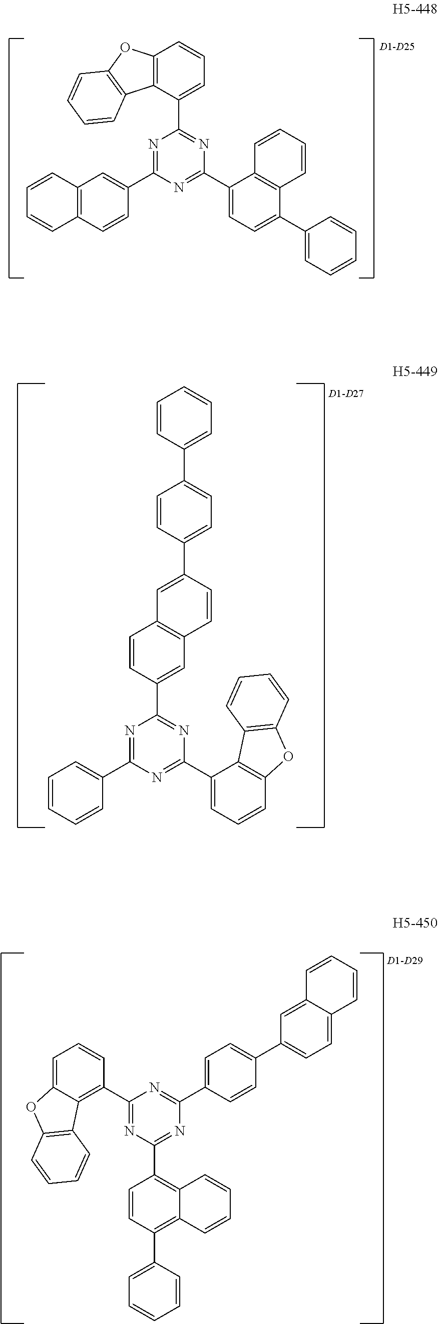

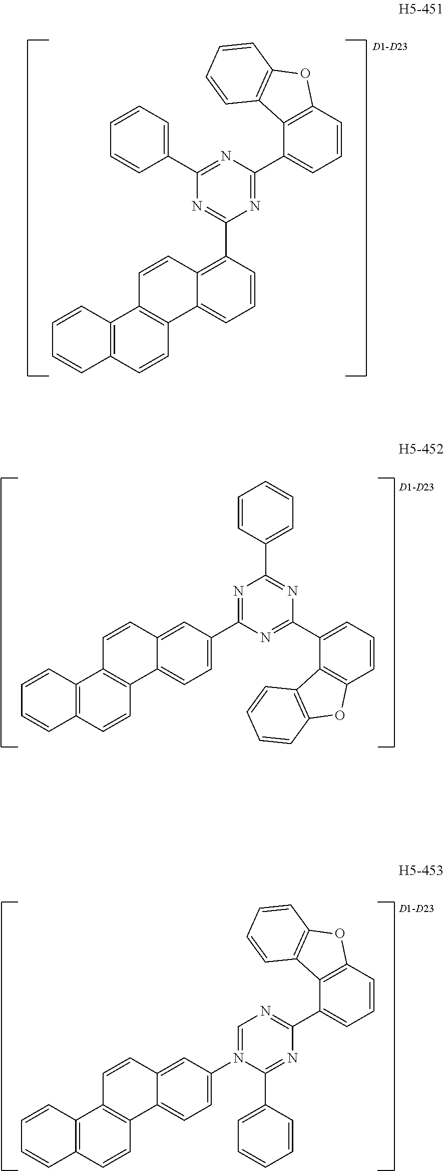

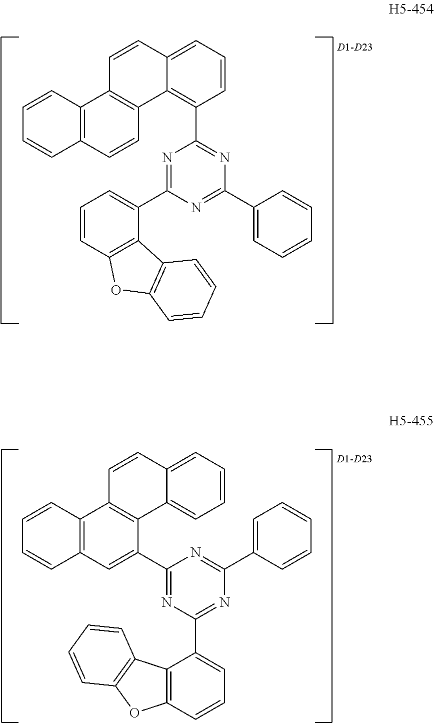

Hereinafter, an organic electroluminescent compound according to one embodiment will be described.

The organic electroluminescent compound according to one embodiment can be represented by any one of the following Formulas 3-1-1 to 3-4-1.

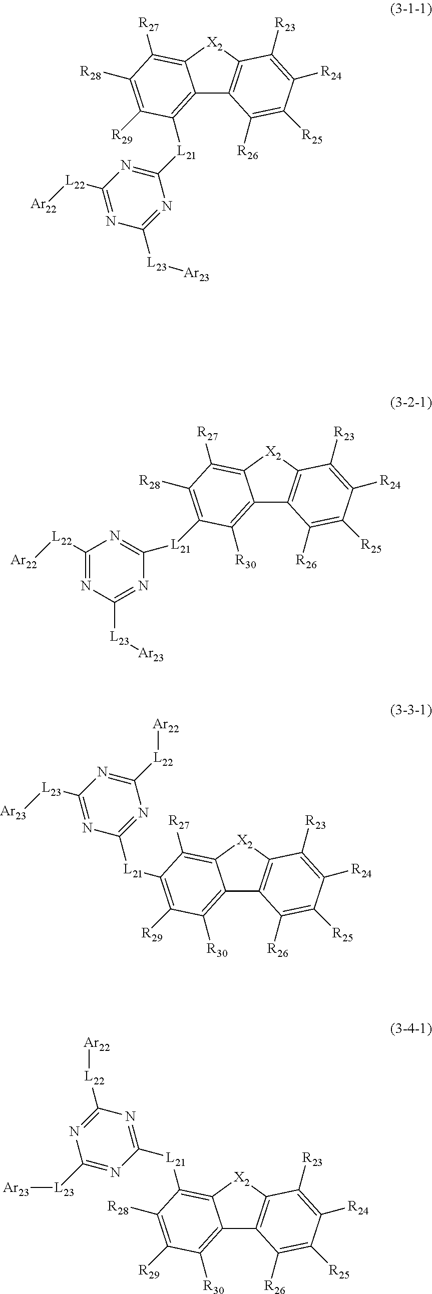

In Formulas 3-1-1 to 3-4-1,
X₂ represents O or S;
L₂₁ and L₂₂ each independently represent a single bond, a substituted or unsubstituted (C₆-C₃₀)arylene, or a substituted or unsubstituted (3- to 30-membered)heteroarylene;
L₂₃ represents phenylene unsubstituted or substituted with deuterium;
Ar₂₂ represents a substituted or unsubstituted (C₆-C₃₀)aryl, or a substituted or unsubstituted (3- to 30-membered)heteroaryl;
Ar₂₃ represents naphthyl substituted with phenyl, which may be further substituted with deuterium; and
R₂₃ to R₃₀ each independently represent hydrogen, deuterium, a halogen, a cyano, a substituted or unsubstituted (C₁-C₃₀)alkyl, a substituted or unsubstituted (C₃-C₃₀)cycloalkyl, a substituted or unsubstituted (C₆-C₃₀)aryl, a substituted or unsubstituted (3- to 30-membered)heteroaryl, a substituted or unsubstituted (C₁-C₃₀)alkoxy, a substituted or unsubstituted tri(C₁-C₃₀)alkylsilyl, a substituted or unsubstituted di(C₁-C₃₀)alkyl(C₆-C₃₀)arylsilyl, a substituted or unsubstituted (C₁-C₃₀)alkyldi(C₆-C₃₀)arylsilyl, a substituted or unsubstituted tri(C₆-C₃₀)arylsilyl, a substituted or unsubstituted mono- or di(C₁-C₃₀)alkylamino, a substituted or unsubstituted mono- or di(C₂-C₃₀)alkenylamino, a substituted or unsubstituted (C₁-C₃₀)alkyl(C₂-C₃₀)alkenylamino, a substituted or unsubstituted mono- or di(C₆-C₃₀)arylamino, a substituted or unsubstituted (C₁-C₃₀)alkyl(C₆-C₃₀)arylamino, a substituted or unsubstituted mono- or di(3- to 30-membered)heteroarylamino, a substituted or unsubstituted (C₁-C₃₀)alkyl(3- to 30-membered)heteroarylamino, a substituted or unsubstituted (C₂-C₃₀)alkenyl(C₆-C₃₀)arylamino, a substituted or unsubstituted (C₂-C₃₀)alkenyl(3- to 30-membered)heteroarylamino, a substituted or unsubstituted (C₆-C₃₀)aryl(3- to 30-membered)heteroarylamino, or a substituted or unsubstituted fused ring of (C₃-C₃₀) aliphatic ring and (C₆-C₃₀) aromatic ring;
provided that in each of Formulas 3-1-1 to 3-4-1, at least one of R₂₃ to R₃₀ is a substituted or unsubstituted (C₆-C₃₀)aryl, or a substituted or unsubstituted (3- to 30-membered)heteroaryl.

In one embodiment, X may be O.

In one embodiment, L₂₁ and L₂₂ each independently may be a single bond.

In one embodiment, Ar₂₂ may be a substituted or unsubstituted (C₆-C₂₅)aryl, preferably (C₆-C₁₈)aryl unsubstituted or substituted with deuterium. For example, Ar₂₂ may be phenyl unsubstituted or substituted with deuterium.

In one embodiment, R₂₃ to R₃₀ each independently may be hydrogen, deuterium, or a substituted or unsubstituted (C₆-C₂₅)aryl, preferably hydrogen, deuterium, or (C₆-C₁₈)aryl unsubstituted or substituted with deuterium. For example, R₂₃ to R₃₀ each independently may be hydrogen, deuterium, or phenyl unsubstituted or substituted with deuterium.

In one embodiment, in each of Formulas 3-1-1 to 3-4-1, at least one of R₂₃ to R₃₀ may be a substituted or unsubstituted (C₆-C₃₀)aryl, preferably (C₆-C₂₅)aryl unsubstituted or substituted with deuterium. For example, in each of Formulas 3-1-1 to 3-4-1, at least one of R₂₃ to R₃₀ may be phenyl unsubstituted or substituted with deuterium.

In one embodiment, Ar₂₃ may be represented by the following Formula A.

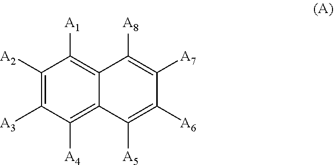

In Formula A, any one of A₁ to A₈ is linked to L₂₃; A₁ to A₈, which is not linked to L₂₃, each independently represent hydrogen, deuterium, or a substituted or unsubstituted phenyl; provide that at least one of A₁ to A₈ is a substituted or unsubstituted phenyl. The above phenyl can be substituted with deuterium.

The compound represented by any one of Formulas 3-1-1 to 3-4-1 according to the present disclosure can be prepared as shown in the following Reaction Scheme 3, but is not limited thereto.

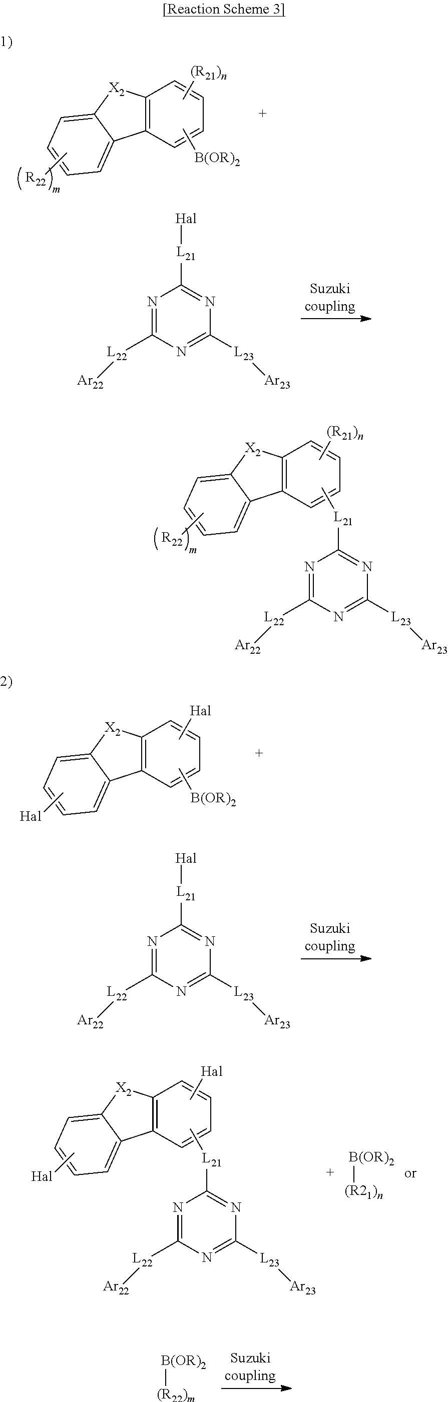

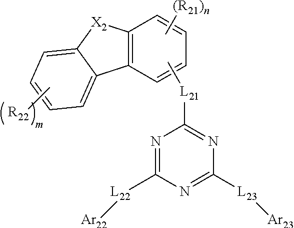

In Reaction Scheme 3, X₂, L₂₁ to L₂₃, Ar₂₂, and Ar₂₃ are as defined in Formulas 3-1-1 to 3-4-1, R₂₁ is as defined for R₂₇ to R₃₀ in Formulas 3-1-1 to 3-4-1, R₂₂ is as defined for R₂₃ to R₂₆ in Formulas 3-1-1 to 3-4-1, Hal means halogen, n is an integer of 1 to 3, m is an integer of 1 to 4, and when each of n and m is an integer of 2 or more, each of R₂₁ and each of R₂₂ may be the same as or different from each other.

According to one embodiment, the compound represented by any one of Formulas 3-1-1 to 3-4-1 may be more specifically illustrated by the following compounds, but is not limited thereto:

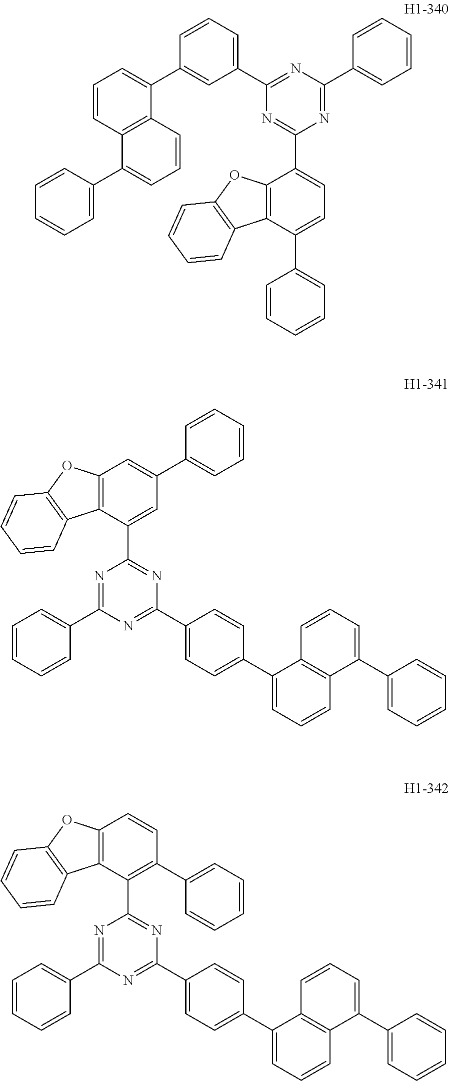 [SMILES: c1ccc(-c2nc(-c3cccc(-c4cccc5c(-c6ccccc6)cccc45)c3)nc(-c3ccc(-c4ccccc4)c4c3oc3ccccc34)n2)cc1 ; c1ccc(-c2cc(-c3nc(-c4ccccc4)nc(-c4ccc(-c5cccc6c(-c7ccccc7)cccc56)cc4)n3)c3c(c2)oc2ccccc23)cc1 ; c1ccc(-c2nc(-c3ccc(-c4cccc5c(-c6ccccc6)cccc45)cc3)nc(-c3c(-c4ccccc4)ccc4oc5ccccc5c34)n2)cc1]

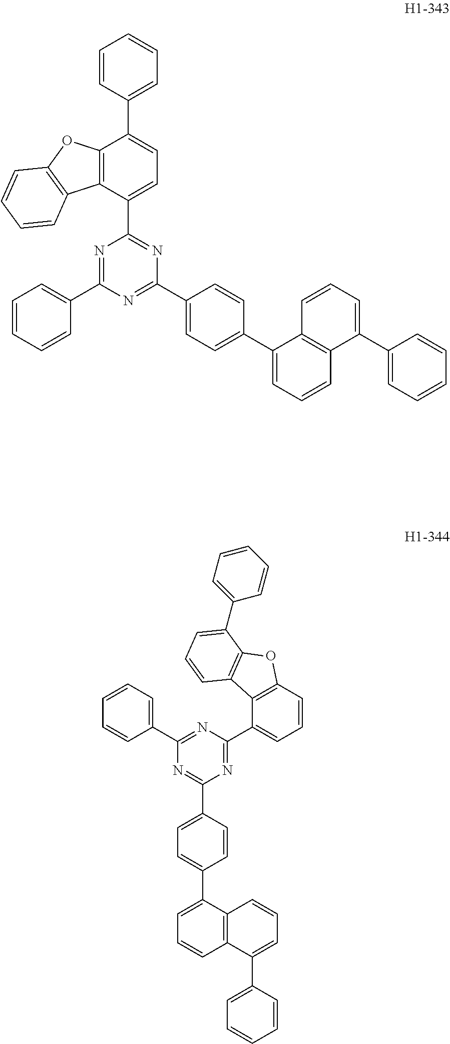 [SMILES: c1ccc(-c2nc(-c3ccc(-c4cccc5c(-c6ccccc6)cccc45)cc3)nc(-c3ccc(-c4ccccc4)c4oc5ccccc5c34)n2)cc1 ; c1ccc(-c2nc(-c3ccc(-c4cccc5c(-c6ccccc6)cccc45)cc3)nc(-c3cccc4oc5c(-c6ccccc6)cccc5c34)n2)cc1]

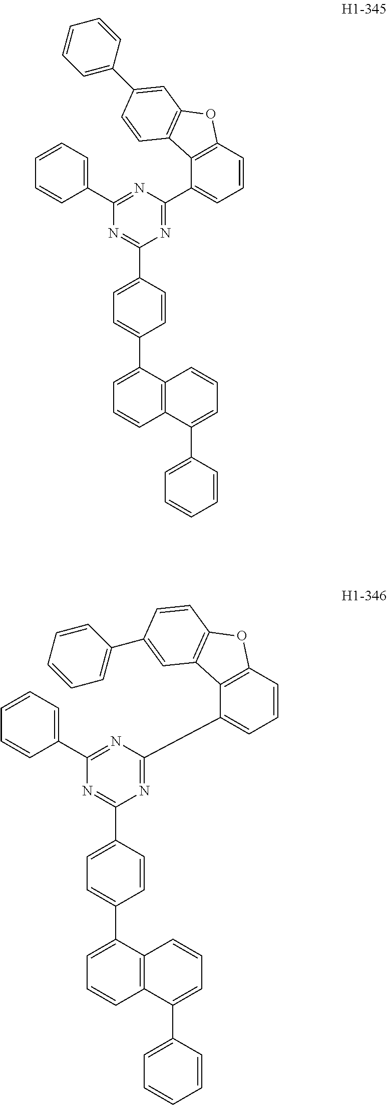 [SMILES: c1ccc(-c2ccc3c(c2)oc2cccc(-c4nc(-c5ccccc5)nc(-c5ccc(-c6cccc7c(-c8ccccc8)cccc67)cc5)n4)c23)cc1 ; c1ccc(-c2ccc3oc4cccc(-c5nc(-c6ccccc6)nc(-c6ccc(-c7cccc8c(-c9ccccc9)cccc78)cc6)n5)c4c3c2)cc1]

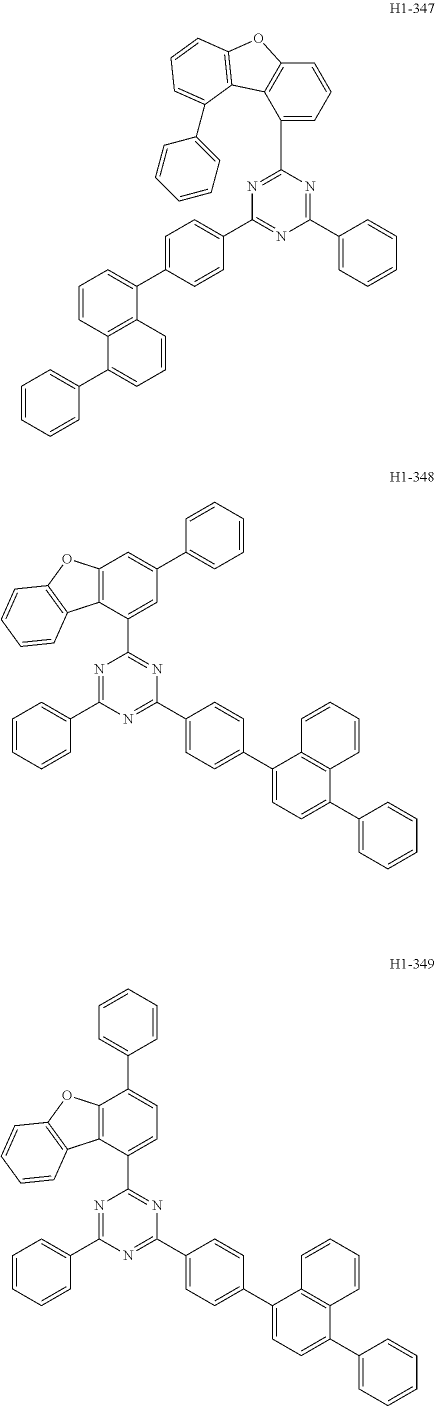 [SMILES: c1ccc(-c2nc(-c3ccc(-c4cccc5c(-c6ccccc6)cccc45)cc3)nc(-c3cccc4oc5cccc(-c6ccccc6)c5c34)n2)cc1 ; c1ccc(-c2cc(-c3nc(-c4ccccc4)nc(-c4ccc(-c5ccc(-c6ccccc6)c6ccccc56)cc4)n3)c3c(c2)oc2ccccc23)cc1 ; c1ccc(-c2nc(-c3ccc(-c4ccc(-c5ccccc5)c5ccccc45)cc3)nc(-c3ccc(-c4ccccc4)c4oc5ccccc5c34)n2)cc1]

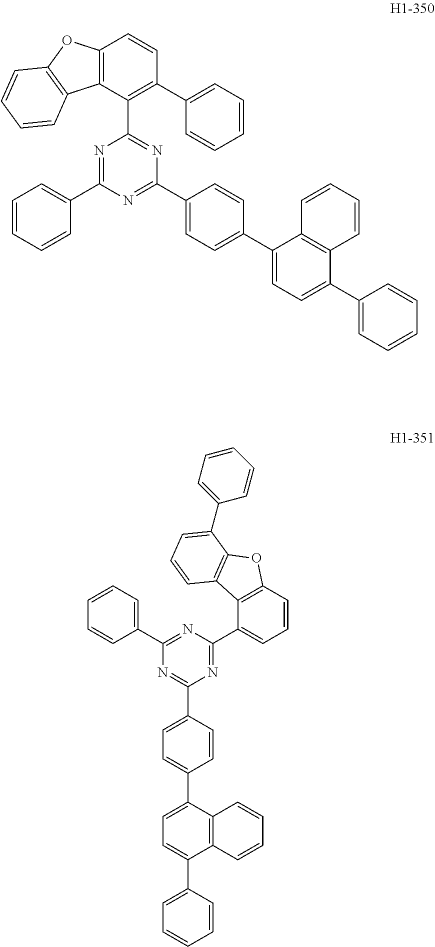 [SMILES: c1ccc(-c2nc(-c3ccc(-c4ccc(-c5ccccc5)c5ccccc45)cc3)nc(-c3c(-c4ccccc4)ccc4oc5ccccc5c34)n2)cc1 ; c1ccc(-c2nc(-c3ccc(-c4ccc(-c5ccccc5)c5ccccc45)cc3)nc(-c3cccc4oc5c(-c6ccccc6)cccc5c34)n2)cc1]

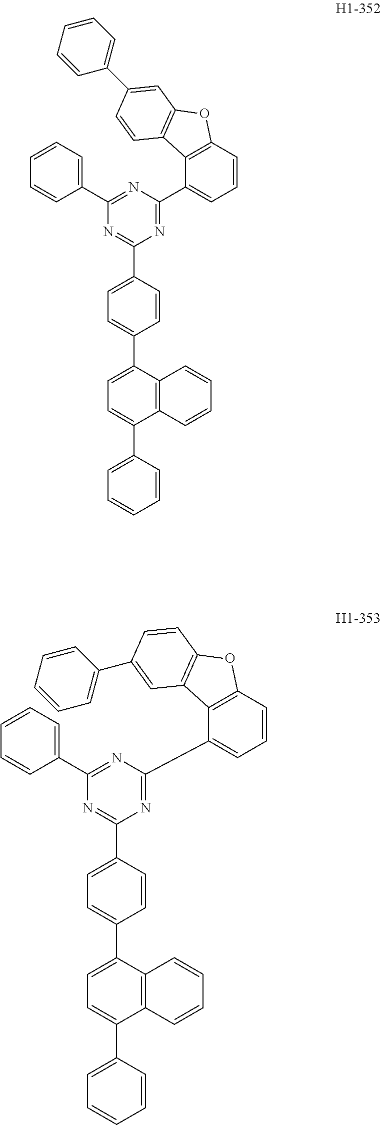 [SMILES: c1ccc(-c2ccc3c(c2)oc2cccc(-c4nc(-c5ccccc5)nc(-c5ccc(-c6ccc(-c7ccccc7)c7ccccc67)cc5)n4)c23)cc1 ; c1ccc(-c2ccc3oc4cccc(-c5nc(-c6ccccc6)nc(-c6ccc(-c7ccc(-c8ccccc8)c8ccccc78)cc6)n5)c4c3c2)cc1]

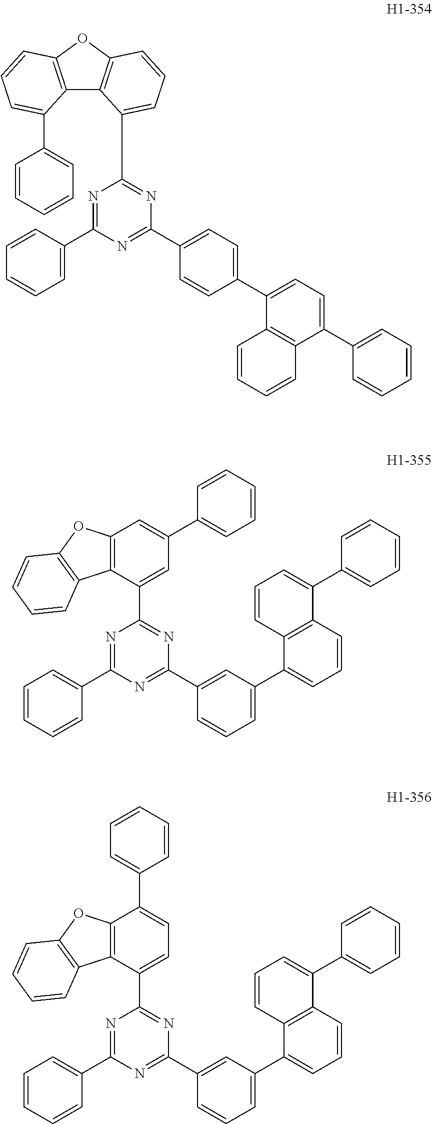 [SMILES: c1ccc(-c2nc(-c3ccc(-c4ccc(-c5ccccc5)c5ccccc45)cc3)nc(-c3cccc4oc5cccc(-c6ccccc6)c5c34)n2)cc1 ; c1ccc(-c2cc(-c3nc(-c4ccccc4)nc(-c4cccc(-c5cccc6c(-c7ccccc7)cccc56)c4)n3)c3c(c2)oc2ccccc23)cc1 ; c1ccc(-c2nc(-c3cccc(-c4cccc5c(-c6ccccc6)cccc45)c3)nc(-c3ccc(-c4ccccc4)c4oc5ccccc5c34)n2)cc1]

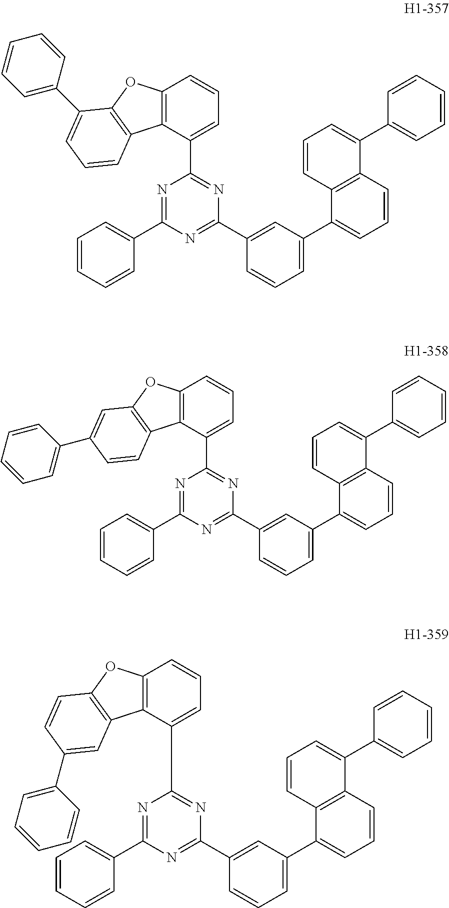 [SMILES: c1ccc(-c2nc(-c3cccc(-c4cccc5c(-c6ccccc6)cccc45)c3)nc(-c3cccc4oc5c(-c6ccccc6)cccc5c34)n2)cc1 ; c1ccc(-c2ccc3c(c2)oc2cccc(-c4nc(-c5ccccc5)nc(-c5cccc(-c6cccc7c(-c8ccccc8)cccc67)c5)n4)c23)cc1 ; c1ccc(-c2ccc3oc4cccc(-c5nc(-c6ccccc6)nc(-c6cccc(-c7cccc8c(-c9ccccc9)cccc78)c6)n5)c4c3c2)cc1]

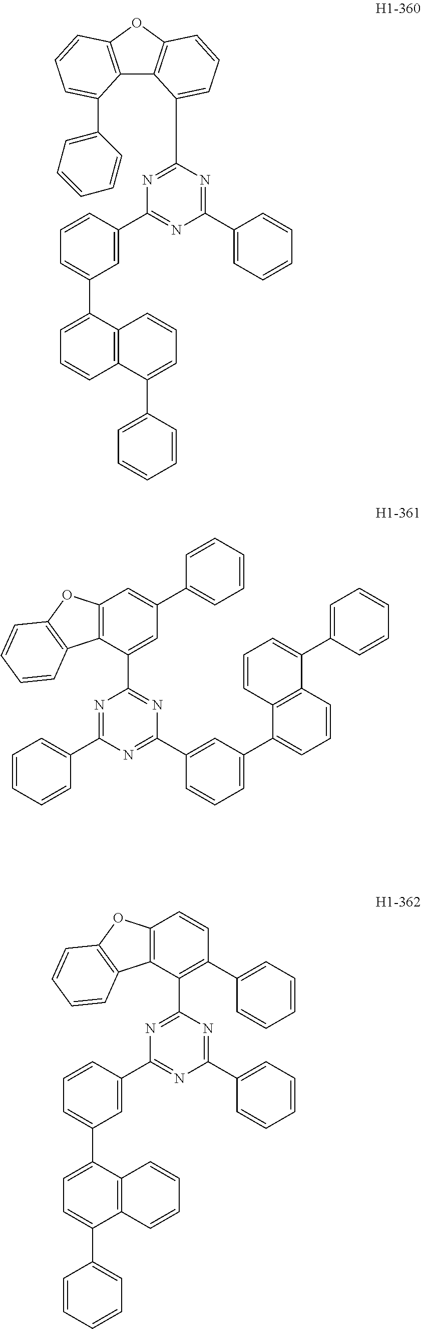 [SMILES: c1ccc(-c2nc(-c3cccc(-c4cccc5c(-c6ccccc6)cccc45)c3)nc(-c3cccc4oc5cccc(-c6ccccc6)c5c34)n2)cc1 ; c1ccc(-c2cc(-c3nc(-c4ccccc4)nc(-c4cccc(-c5cccc6c(-c7ccccc7)cccc56)c4)n3)c3c(c2)oc2ccccc23)cc1 ; c1ccc(-c2nc(-c3cccc(-c4ccc(-c5ccccc5)c5ccccc45)c3)nc(-c3c(-c4ccccc4)ccc4oc5ccccc5c34)n2)cc1]

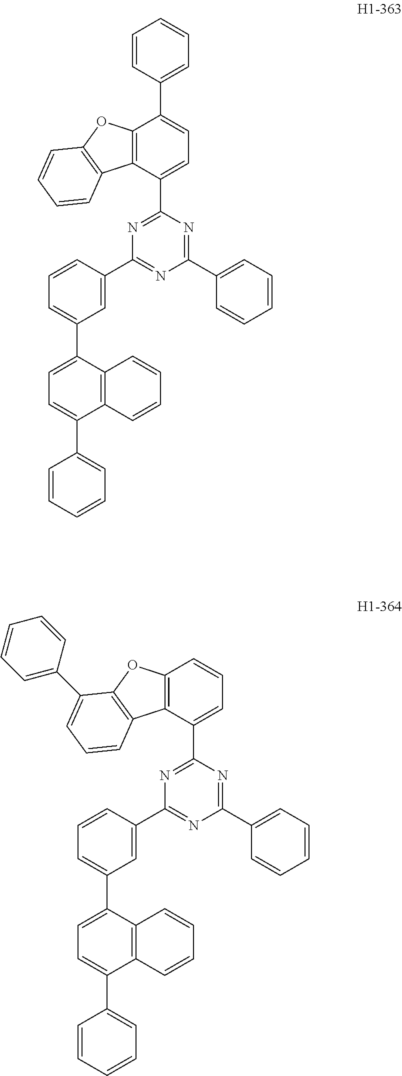 [SMILES: c1ccc(-c2nc(-c3cccc(-c4ccc(-c5ccccc5)c5ccccc45)c3)nc(-c3ccc(-c4ccccc4)c4oc5ccccc5c34)n2)cc1 ; c1ccc(-c2nc(-c3cccc(-c4ccc(-c5ccccc5)c5ccccc45)c3)nc(-c3cccc4oc5c(-c6ccccc6)cccc5c34)n2)cc1]

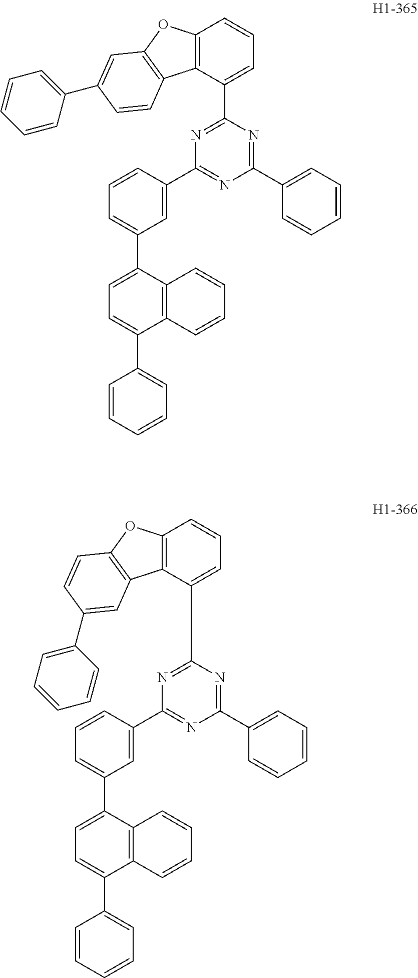 [SMILES: c1ccc(-c2ccc3c(c2)oc2cccc(-c4nc(-c5ccccc5)nc(-c5cccc(-c6ccc(-c7ccccc7)c7ccccc67)c5)n4)c23)cc1 ; c1ccc(-c2ccc3oc4cccc(-c5nc(-c6ccccc6)nc(-c6cccc(-c7ccc(-c8ccccc8)c8ccccc78)c6)n5)c4c3c2)cc1]

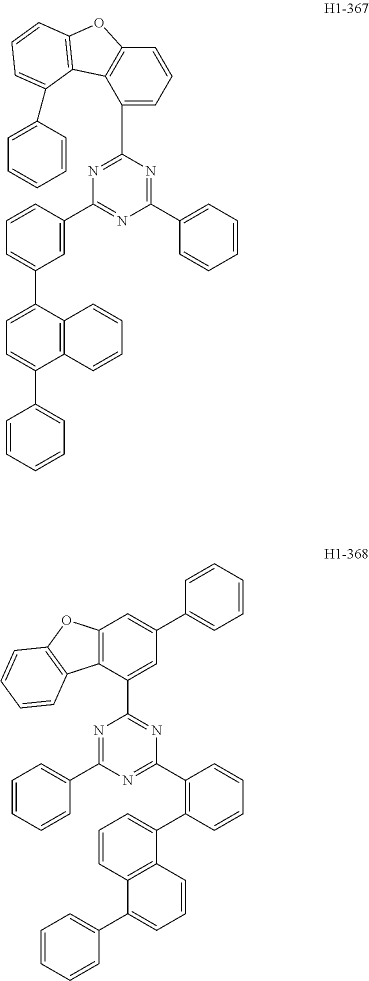 [SMILES: c1ccc(-c2nc(-c3cccc(-c4ccc(-c5ccccc5)c5ccccc45)c3)nc(-c3cccc4oc5cccc(-c6ccccc6)c5c34)n2)cc1 ; c1ccc(-c2cc(-c3nc(-c4ccccc4)nc(-c4ccccc4-c4cccc5c(-c6ccccc6)cccc45)n3)c3c(c2)oc2ccccc23)cc1]

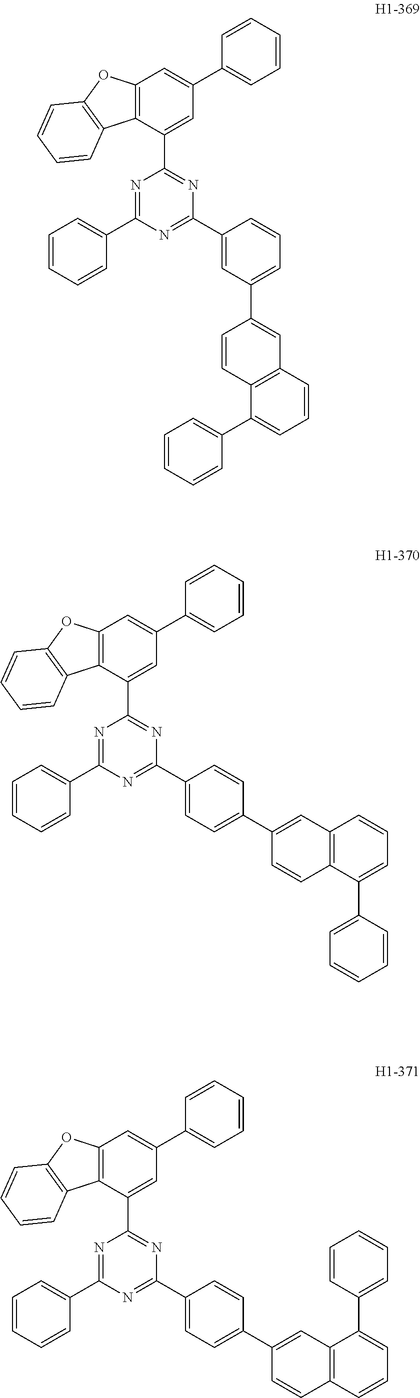 [SMILES: c1ccc(-c2cc(-c3nc(-c4ccccc4)nc(-c4cccc(-c5ccc6c(-c7ccccc7)cccc6c5)c4)n3)c3c(c2)oc2ccccc23)cc1 ; c1ccc(-c2cc(-c3nc(-c4ccccc4)nc(-c4ccc(-c5ccc6c(-c7ccccc7)cccc6c5)cc4)n3)c3c(c2)oc2ccccc23)cc1 ; c1ccc(-c2cc(-c3nc(-c4ccccc4)nc(-c4ccc(-c5ccc6cccc(-c7ccccc7)c6c5)cc4)n3)c3c(c2)oc2ccccc23)cc1]

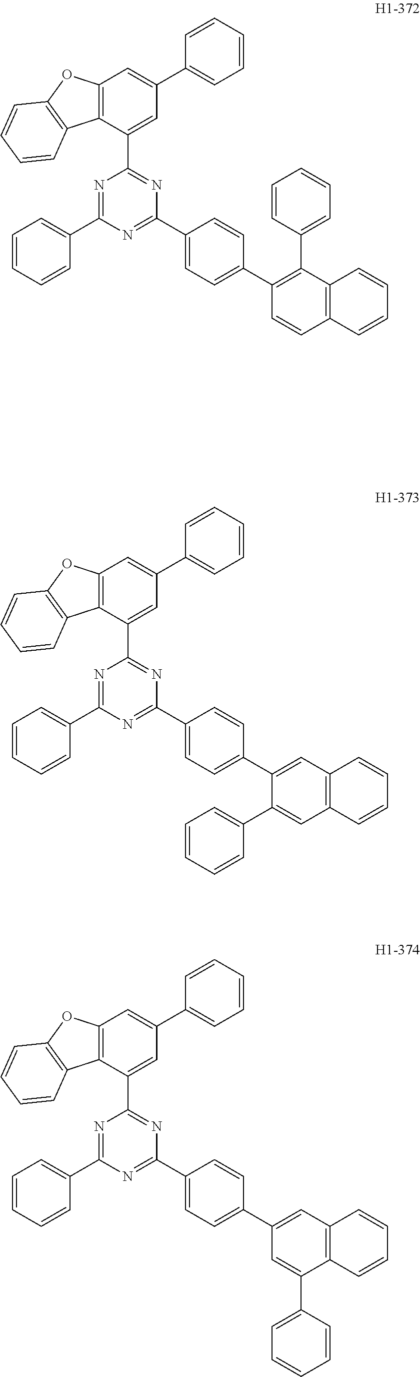 [SMILES: c1ccc(-c2cc(-c3nc(-c4ccccc4)nc(-c4ccc(-c5ccc6ccccc6c5-c5ccccc5)cc4)n3)c3c(c2)oc2ccccc23)cc1 ; c1ccc(-c2cc(-c3nc(-c4ccccc4)nc(-c4ccc(-c5cc6ccccc6cc5-c5ccccc5)cc4)n3)c3c(c2)oc2ccccc23)cc1 ; c1ccc(-c2cc(-c3nc(-c4ccccc4)nc(-c4ccc(-c5cc(-c6ccccc6)c6ccccc6c5)cc4)n3)c3c(c2)oc2ccccc23)cc1]

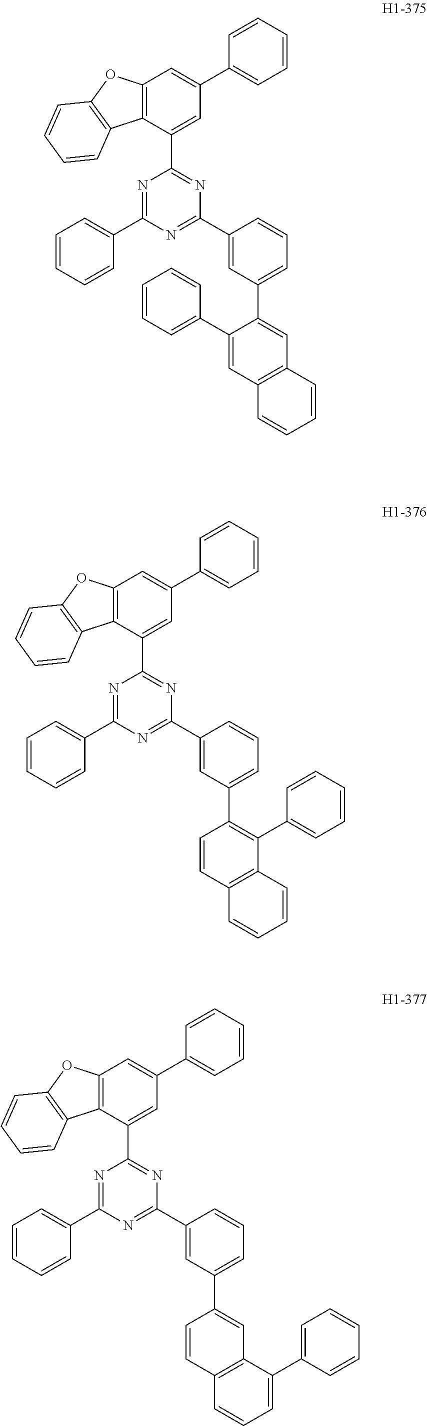 [SMILES: c1ccc(-c2cc(-c3nc(-c4ccccc4)nc(-c4cccc(-c5cc6ccccc6cc5-c5ccccc5)c4)n3)c3c(c2)oc2ccccc23)cc1 ; c1ccc(-c2cc(-c3nc(-c4ccccc4)nc(-c4cccc(-c5ccc6ccccc6c5-c5ccccc5)c4)n3)c3c(c2)oc2ccccc23)cc1 ; c1ccc(-c2cc(-c3nc(-c4ccccc4)nc(-c4cccc(-c5ccc6cccc(-c7ccccc7)c6c5)c4)n3)c3c(c2)oc2ccccc23)cc1]

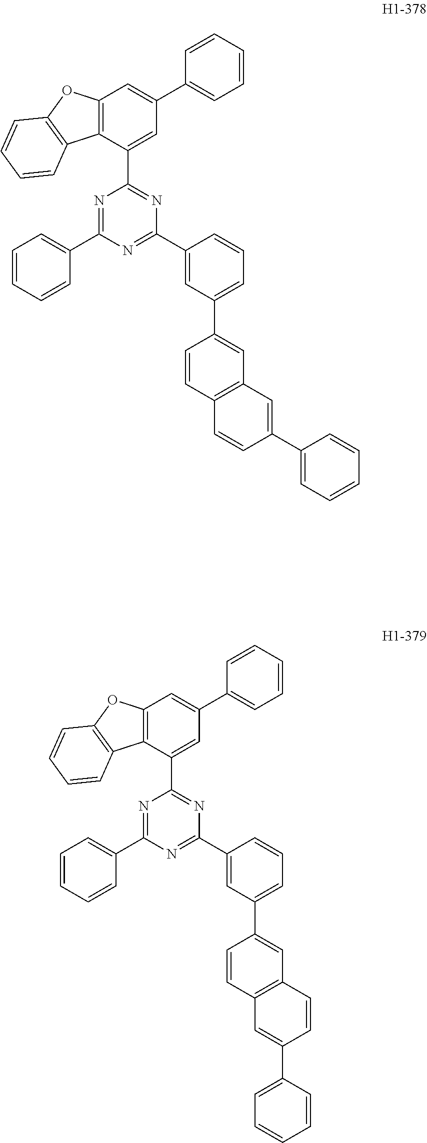 [SMILES: c1ccc(-c2ccc3ccc(-c4cccc(-c5nc(-c6ccccc6)nc(-c6cc(-c7ccccc7)cc7oc8ccccc8c67)n5)c4)cc3c2)cc1 ; c1ccc(-c2ccc3cc(-c4cccc(-c5nc(-c6ccccc6)nc(-c6cc(-c7ccccc7)cc7oc8ccccc8c67)n5)c4)ccc3c2)cc1]

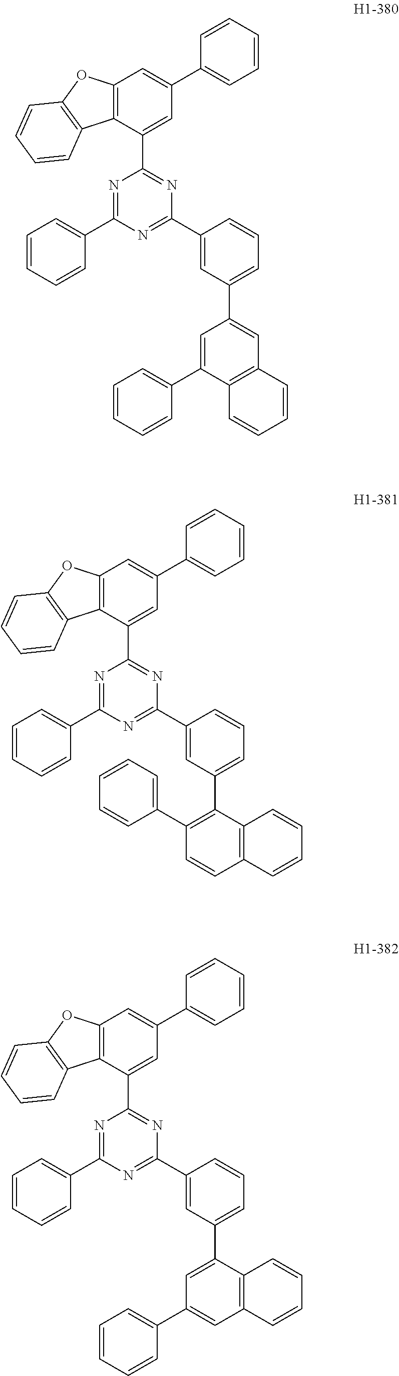 [SMILES: c1ccc(-c2cc(-c3nc(-c4ccccc4)nc(-c4cccc(-c5cc(-c6ccccc6)c6ccccc6c5)c4)n3)c3c(c2)oc2ccccc23)cc1 ; c1ccc(-c2cc(-c3nc(-c4ccccc4)nc(-c4cccc(-c5c(-c6ccccc6)ccc6ccccc56)c4)n3)c3c(c2)oc2ccccc23)cc1 ; c1ccc(-c2cc(-c3cccc(-c4nc(-c5ccccc5)nc(-c5cc(-c6ccccc6)cc6oc7ccccc7c56)n4)c3)c3ccccc3c2)cc1]

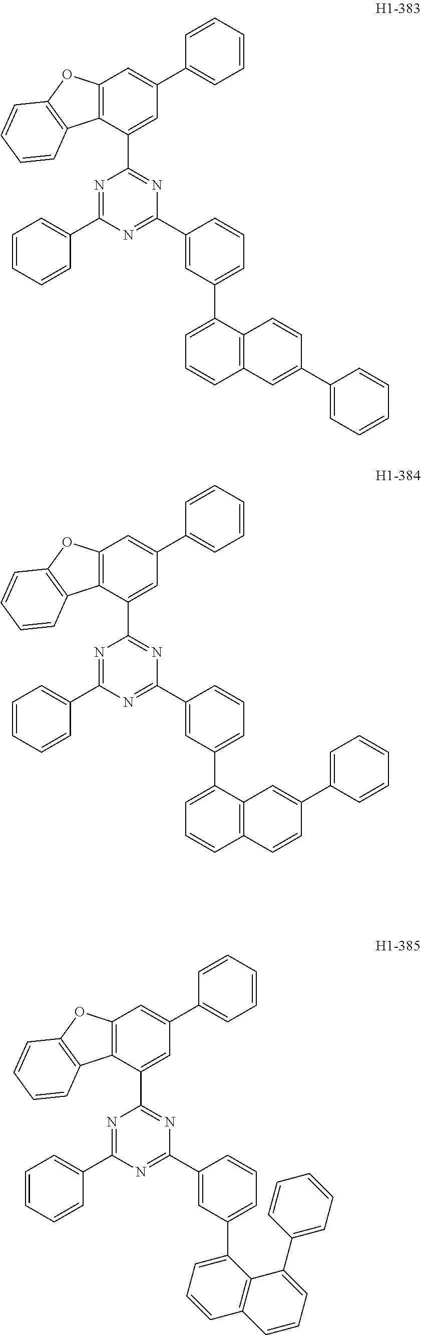 [SMILES: c1ccc(-c2ccc3c(-c4cccc(-c5nc(-c6ccccc6)nc(-c6cc(-c7ccccc7)cc7oc8ccccc8c67)n5)c4)cccc3c2)cc1 ; c1ccc(-c2cc(-c3nc(-c4ccccc4)nc(-c4cccc(-c5cccc6ccc(-c7ccccc7)cc56)c4)n3)c3c(c2)oc2ccccc23)cc1 ; c1ccc(-c2cc(-c3nc(-c4ccccc4)nc(-c4cccc(-c5cccc6cccc(-c7ccccc7)c56)c4)n3)c3c(c2)oc2ccccc23)cc1]

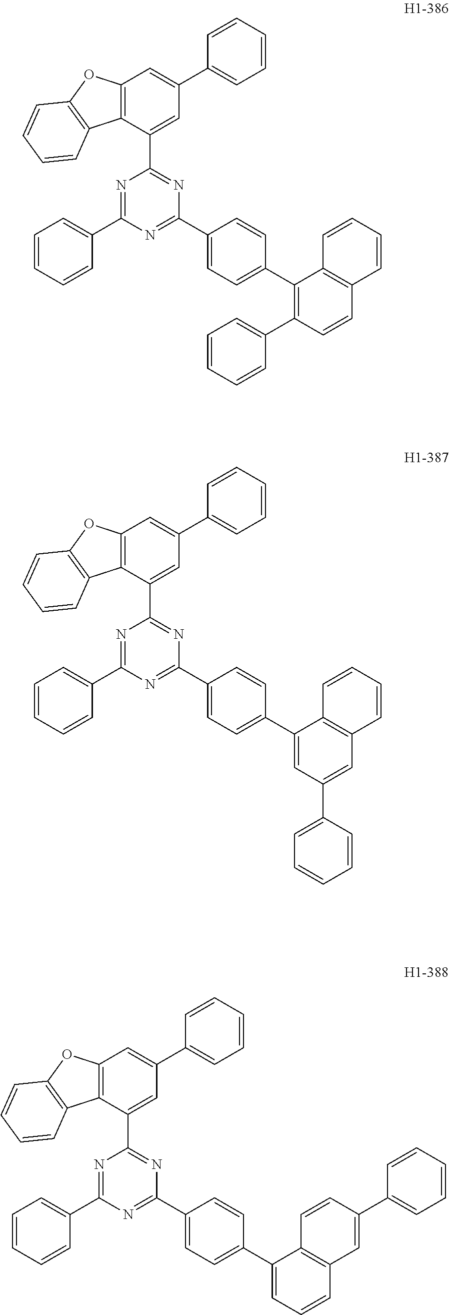 [SMILES: c1ccc(-c2cc(-c3nc(-c4ccccc4)nc(-c4ccc(-c5c(-c6ccccc6)ccc6ccccc56)cc4)n3)c3c(c2)oc2ccccc23)cc1 ; c1ccc(-c2cc(-c3ccc(-c4nc(-c5ccccc5)nc(-c5cc(-c6ccccc6)cc6oc7ccccc7c56)n4)cc3)c3ccccc3c2)cc1 ; c1ccc(-c2ccc3c(-c4ccc(-c5nc(-c6ccccc6)nc(-c6cc(-c7ccccc7)cc7oc8ccccc8c67)n5)cc4)cccc3c2)cc1]

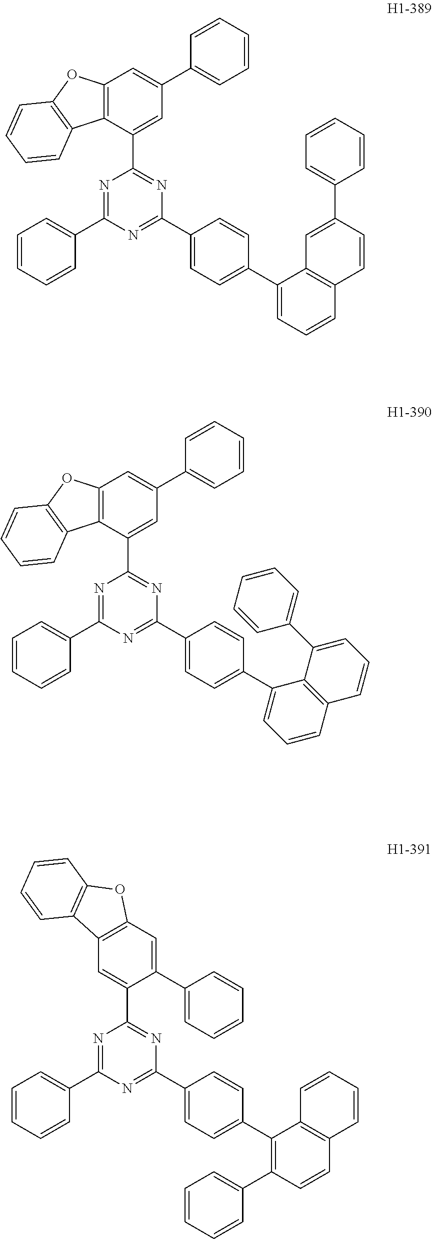 [SMILES: c1ccc(-c2cc(-c3nc(-c4ccccc4)nc(-c4ccc(-c5cccc6ccc(-c7ccccc7)cc56)cc4)n3)c3c(c2)oc2ccccc23)cc1 ; c1ccc(-c2cc(-c3nc(-c4ccccc4)nc(-c4ccc(-c5cccc6cccc(-c7ccccc7)c56)cc4)n3)c3c(c2)oc2ccccc23)cc1 ; c1ccc(-c2nc(-c3ccc(-c4c(-c5ccccc5)ccc5ccccc45)cc3)nc(-c3cc4c(cc3-c3ccccc3)oc3ccccc34)n2)cc1]

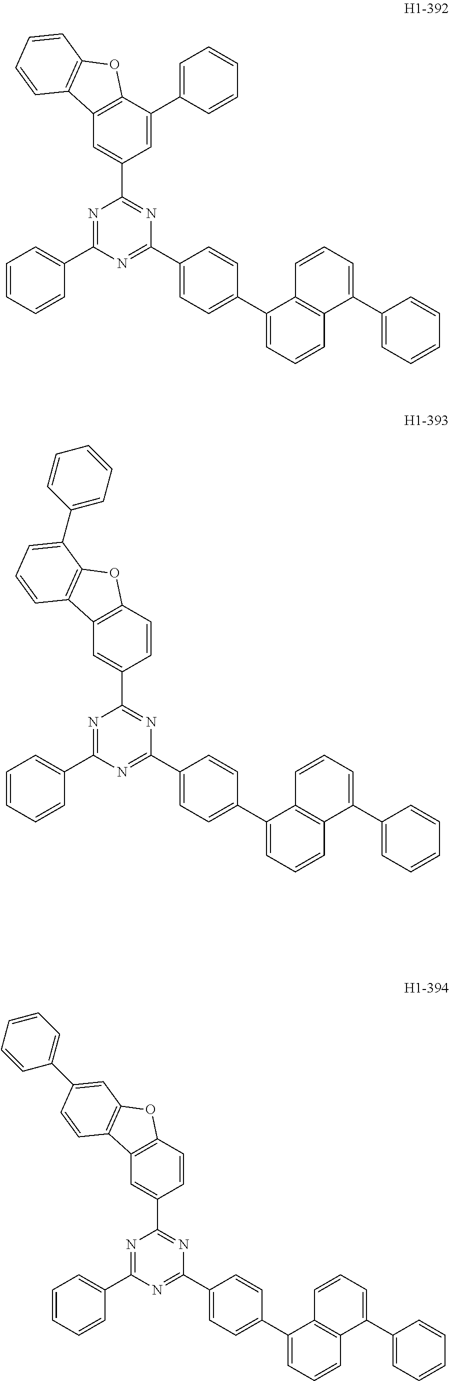 [SMILES: c1ccc(-c2nc(-c3ccc(-c4cccc5c(-c6ccccc6)cccc45)cc3)nc(-c3cc(-c4ccccc4)c4oc5ccccc5c4c3)n2)cc1 ; c1ccc(-c2nc(-c3ccc(-c4cccc5c(-c6ccccc6)cccc45)cc3)nc(-c3ccc4oc5c(-c6ccccc6)cccc5c4c3)n2)cc1 ; c1ccc(-c2ccc3c(c2)oc2ccc(-c4nc(-c5ccccc5)nc(-c5ccc(-c6cccc7c(-c8ccccc8)cccc67)cc5)n4)cc23)cc1]

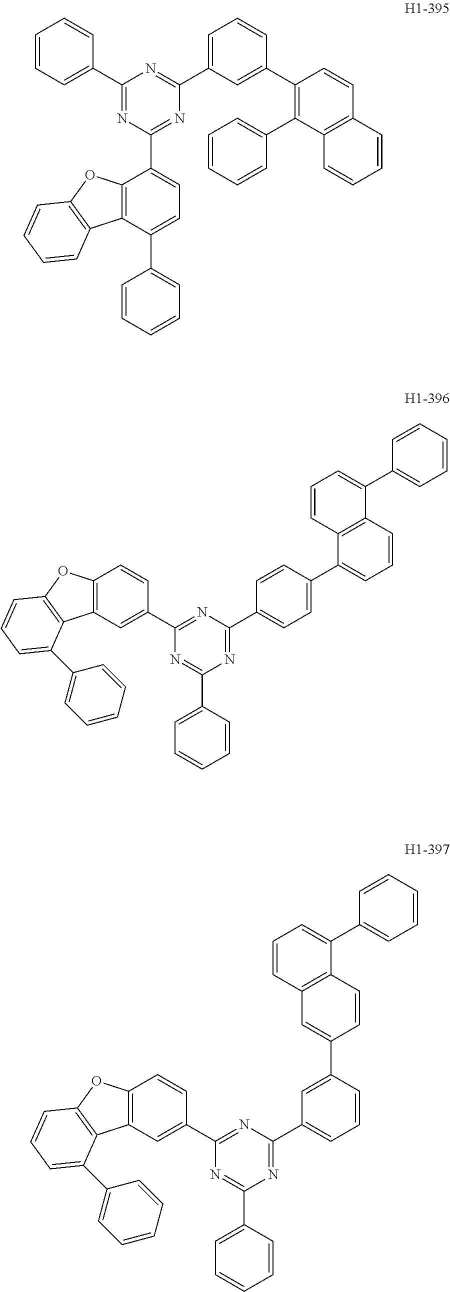 [SMILES: c1ccc(-c2nc(-c3cccc(-c4ccc5ccccc5c4-c4ccccc4)c3)nc(-c3ccc(-c4ccccc4)c4c3oc3ccccc34)n2)cc1 ; c1ccc(-c2nc(-c3ccc(-c4cccc5c(-c6ccccc6)cccc45)cc3)nc(-c3ccc4oc5cccc(-c6ccccc6)c5c4c3)n2)cc1 ; c1ccc(-c2nc(-c3cccc(-c4ccc5c(-c6ccccc6)cccc5c4)c3)nc(-c3ccc4oc5cccc(-c6ccccc6)c5c4c3)n2)cc1]

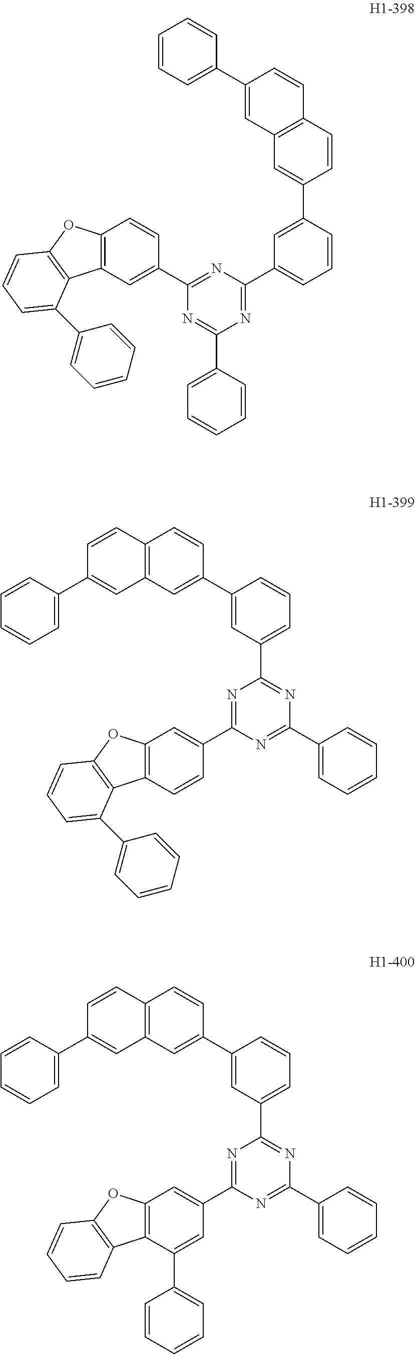 [SMILES: c1ccc(-c2ccc3ccc(-c4cccc(-c5nc(-c6ccccc6)nc(-c6ccc7oc8cccc(-c9ccccc9)c8c7c6)n5)c4)cc3c2)cc1 ; c1ccc(-c2ccc3ccc(-c4cccc(-c5nc(-c6ccccc6)nc(-c6ccc7c(c6)oc6cccc(-c8ccccc8)c67)n5)c4)cc3c2)cc1 ; c1ccc(-c2ccc3ccc(-c4cccc(-c5nc(-c6ccccc6)nc(-c6cc(-c7ccccc7)c7c(c6)oc6ccccc67)n5)c4)cc3c2)cc1]

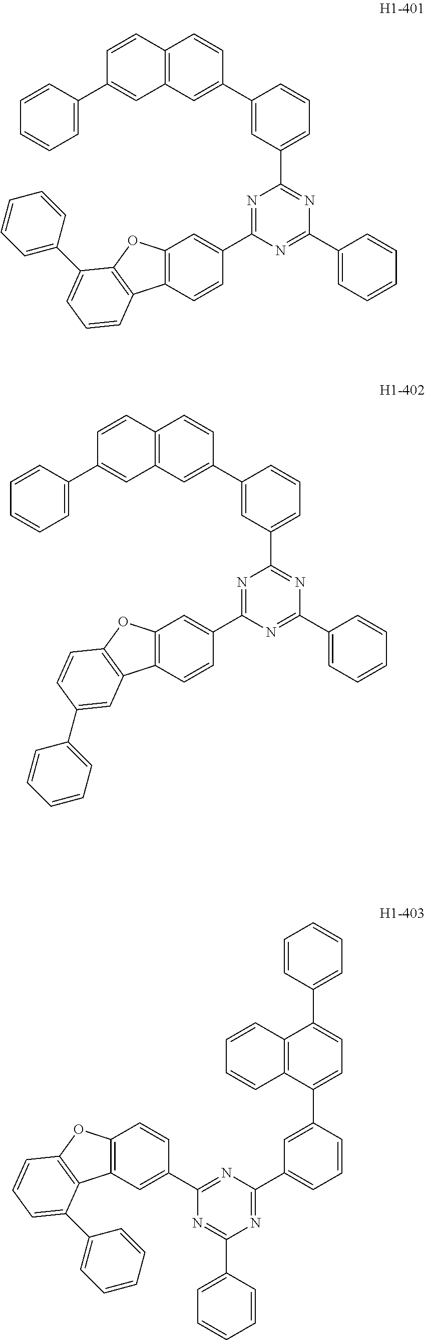 [SMILES: c1ccc(-c2ccc3ccc(-c4cccc(-c5nc(-c6ccccc6)nc(-c6ccc7c(c6)oc6c(-c8ccccc8)cccc67)n5)c4)cc3c2)cc1 ; c1ccc(-c2ccc3ccc(-c4cccc(-c5nc(-c6ccccc6)nc(-c6ccc7c(c6)oc6ccc(-c8ccccc8)cc67)n5)c4)cc3c2)cc1 ; c1ccc(-c2nc(-c3cccc(-c4ccc(-c5ccccc5)c5ccccc45)c3)nc(-c3ccc4oc5cccc(-c6ccccc6)c5c4c3)n2)cc1]

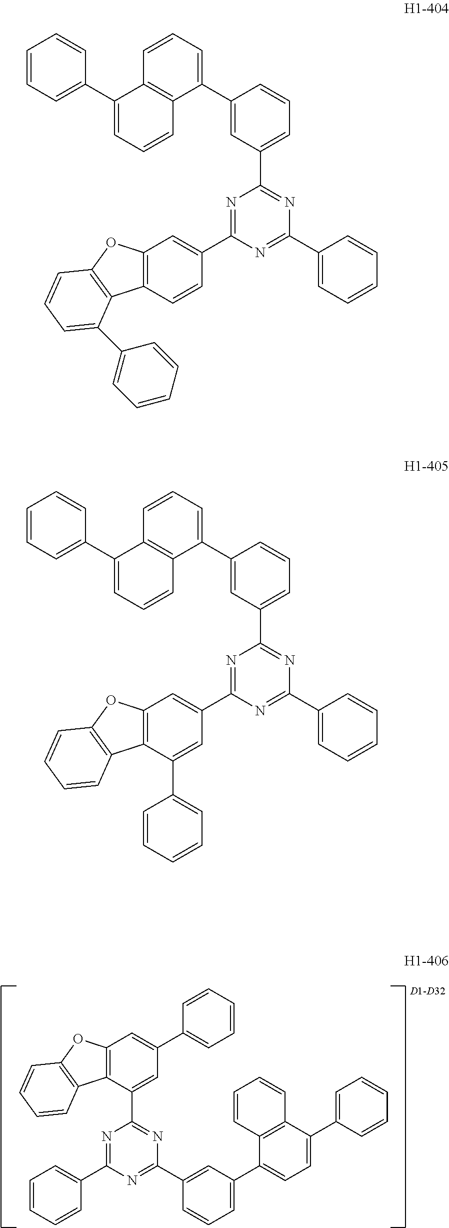

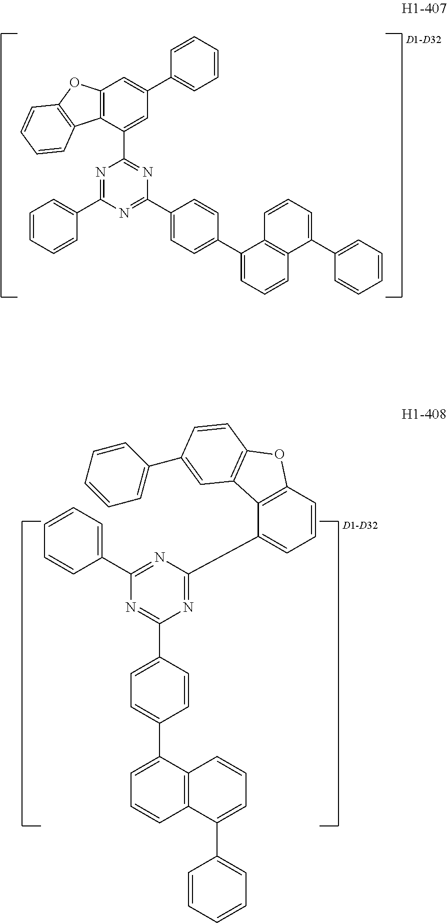

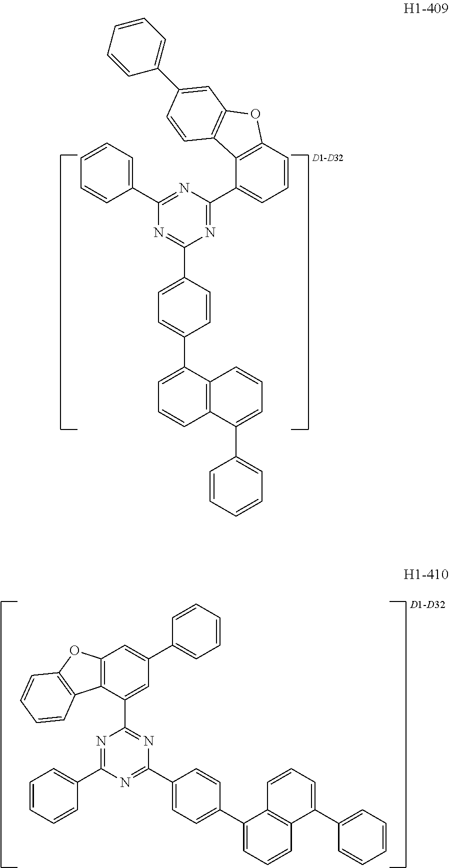

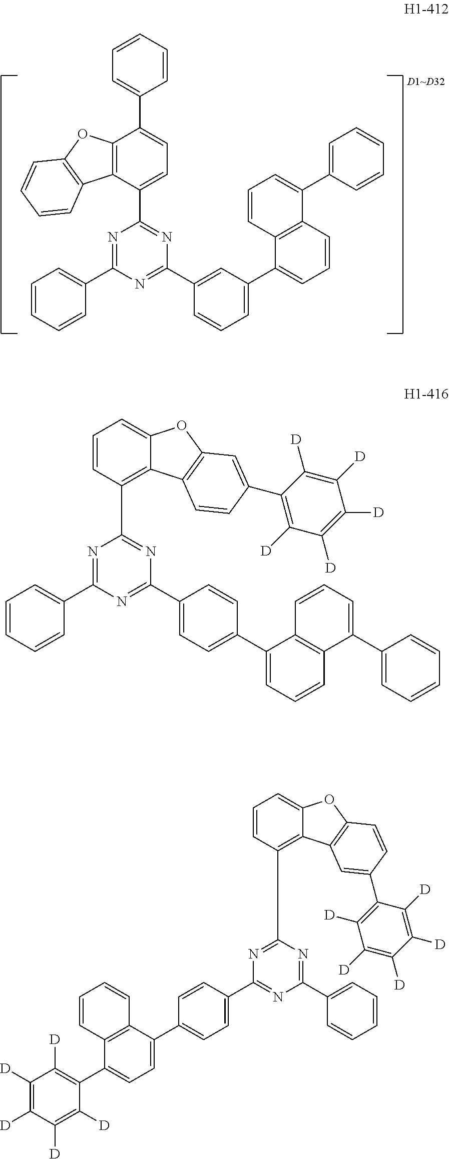

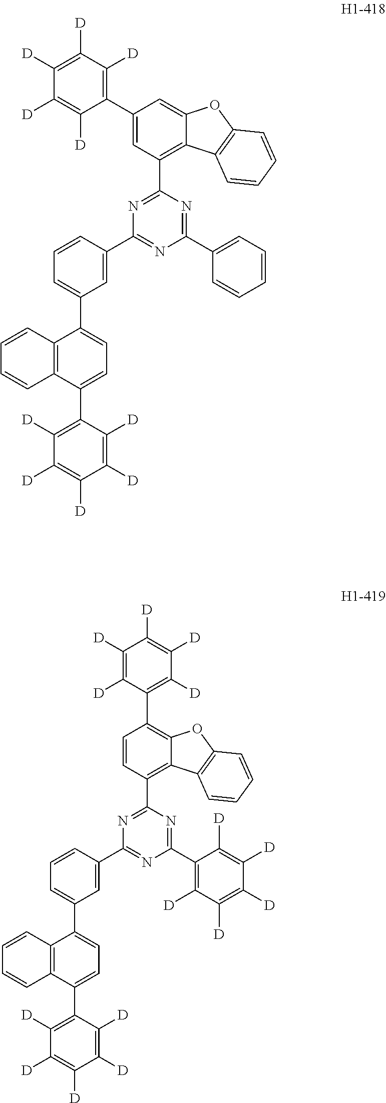 [SMILES: [2H]c1c([2H])c([2H])c(-c2cc(-c3nc(-c4ccccc4)nc(-c4cccc(-c5ccc(-c6c([2H])c([2H])c([2H])c([2H])c6[2H])c6ccccc56)c4)n3)c3c(c2)oc2ccccc23)c([2H])c1[2H] ; [2H]c1c([2H])c([2H])c(-c2nc(-c3cccc(-c4ccc(-c5c([2H])c([2H])c([2H])c([2H])c5[2H])c5ccccc45)c3)nc(-c3ccc(-c4c([2H])c([2H])c([2H])c([2H])c4[2H])c4oc5ccccc5c34)n2)c([2H])c1[2H]]

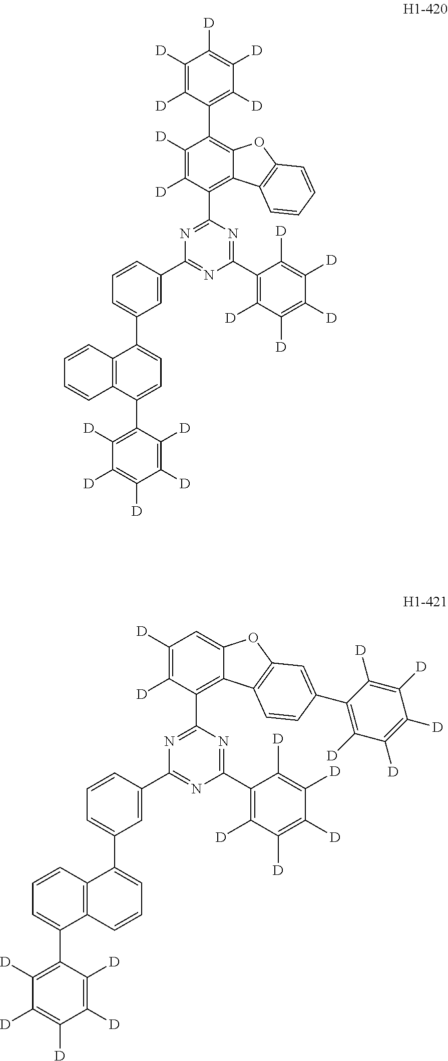 [SMILES: [2H]c1c([2H])c([2H])c(-c2nc(-c3cccc(-c4ccc(-c5c([2H])c([2H])c([2H])c([2H])c5[2H])c5ccccc45)c3)nc(-c3c([2H])c([2H])c(-c4c([2H])c([2H])c([2H])c([2H])c4[2H])c4oc5ccccc5c34)n2)c([2H])c1[2H] ; [2H]c1cc2oc3cc(-c4c([2H])c([2H])c([2H])c([2H])c4[2H])ccc3c2c(-c2nc(-c3cccc(-c4cccc5c(-c6c([2H])c([2H])c([2H])c([2H])c6[2H])cccc45)c3)nc(-c3c([2H])c([2H])c([2H])c([2H])c3[2H])n2)c1[2H]]

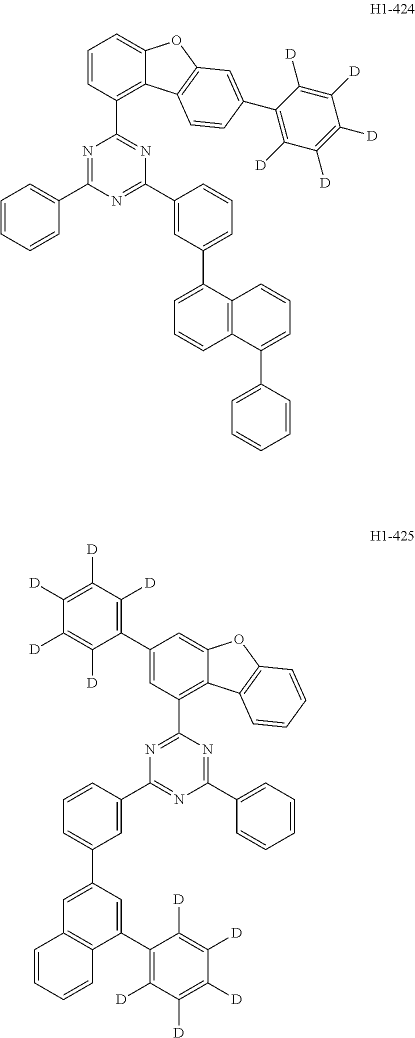 [SMILES: [2H]c1c([2H])c([2H])c(-c2ccc3c(c2)oc2cccc(-c4nc(-c5ccccc5)nc(-c5cccc(-c6cccc7c(-c8ccccc8)cccc67)c5)n4)c23)c([2H])c1[2H] ; [2H]c1c([2H])c([2H])c(-c2cc(-c3nc(-c4ccccc4)nc(-c4cccc(-c5cc(-c6c([2H])c([2H])c([2H])c([2H])c6[2H])c6ccccc6c5)c4)n3)c3c(c2)oc2ccccc23)c([2H])c1[2H]]

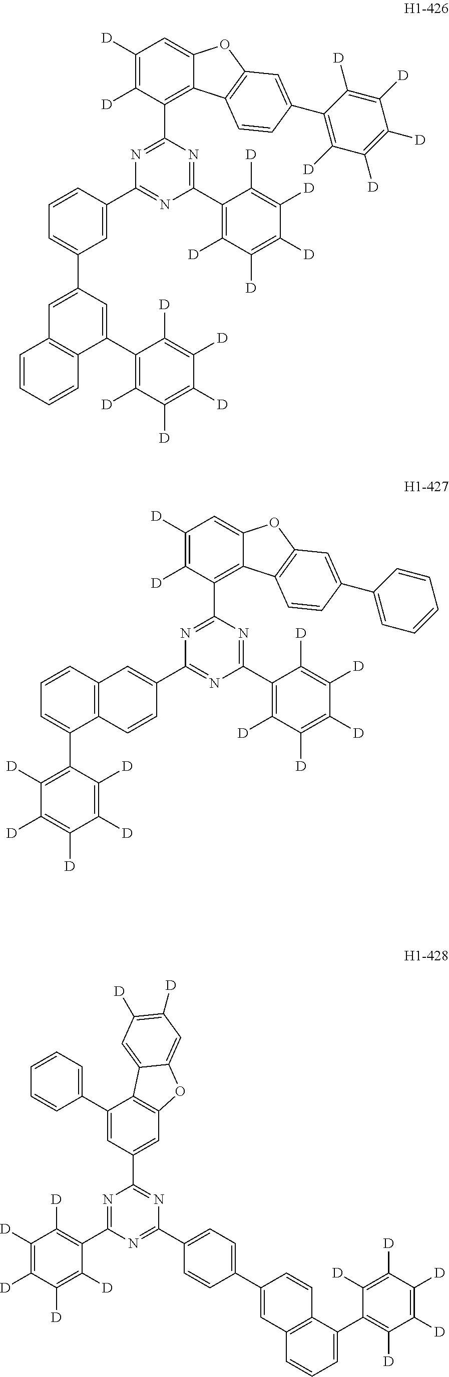 [SMILES: [2H]c1cc2oc3cc(-c4c([2H])c([2H])c([2H])c([2H])c4[2H])ccc3c2c(-c2nc(-c3cccc(-c4cc(-c5c([2H])c([2H])c([2H])c([2H])c5[2H])c5ccccc5c4)c3)nc(-c3c([2H])c([2H])c([2H])c([2H])c3[2H])n2)c1[2H] ; [2H]c1cc2oc3cc(-c4ccccc4)ccc3c2c(-c2nc(-c3ccc4c(-c5c([2H])c([2H])c([2H])c([2H])c5[2H])cccc4c3)nc(-c3c([2H])c([2H])c([2H])c([2H])c3[2H])n2)c1[2H] ; [2H]c1cc2oc3cc(-c4nc(-c5ccc(-c6ccc7c(-c8c([2H])c([2H])c([2H])c([2H])c8[2H])cccc7c6)cc5)nc(-c5c([2H])c([2H])c([2H])c([2H])c5[2H])n4)cc(-c4ccccc4)c3c2cc1[2H]]

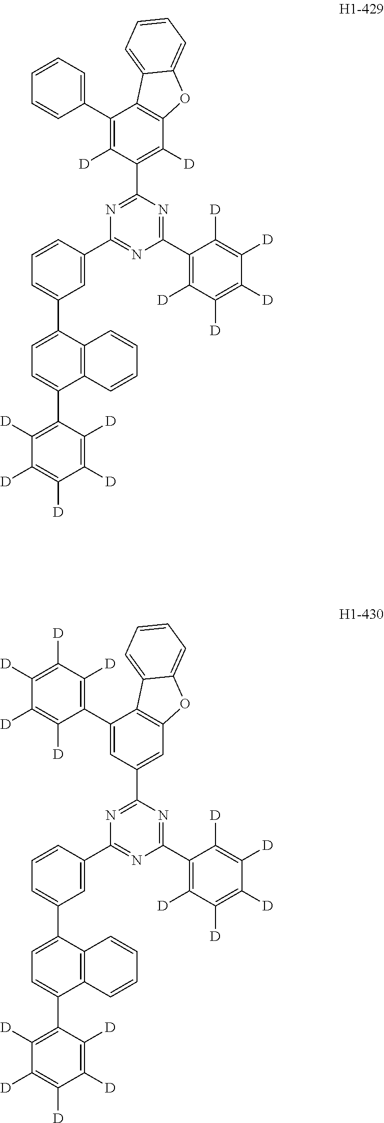 [SMILES: [2H]c1c([2H])c([2H])c(-c2nc(-c3cccc(-c4ccc(-c5c([2H])c([2H])c([2H])c([2H])c5[2H])c5ccccc45)c3)nc(-c3c([2H])c(-c4ccccc4)c4c(oc5ccccc54)c3[2H])n2)c([2H])c1[2H] ; [2H]c1c([2H])c([2H])c(-c2nc(-c3cccc(-c4ccc(-c5c([2H])c([2H])c([2H])c([2H])c5[2H])c5ccccc45)c3)nc(-c3cc(-c4c([2H])c([2H])c([2H])c([2H])c4[2H])c4c(c3)oc3ccccc34)n2)c([2H])c1[2H]]

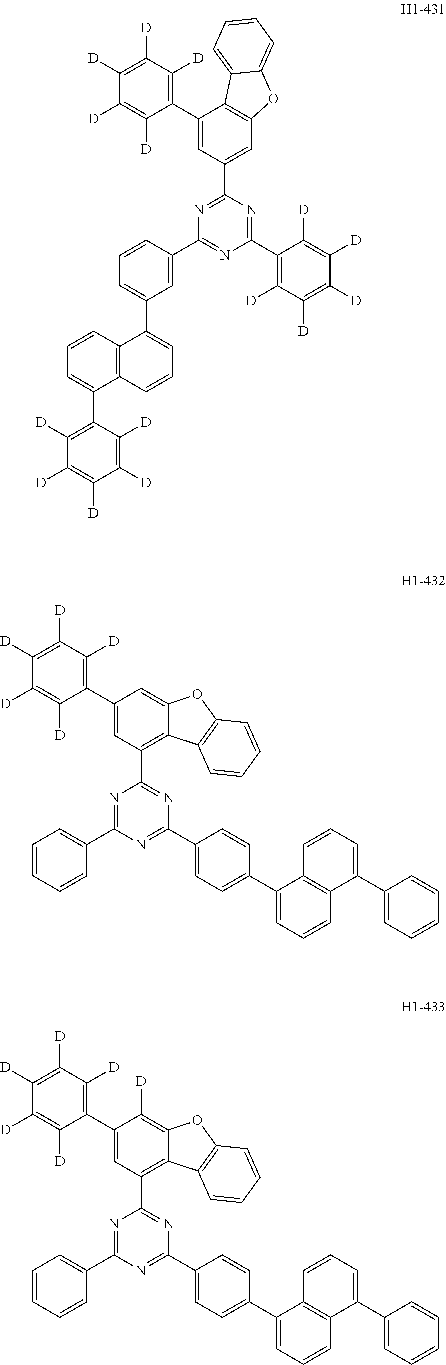 [SMILES: [2H]c1c([2H])c([2H])c(-c2nc(-c3cccc(-c4cccc5c(-c6c([2H])c([2H])c([2H])c([2H])c6[2H])cccc45)c3)nc(-c3cc(-c4c([2H])c([2H])c([2H])c([2H])c4[2H])c4c(c3)oc3ccccc34)n2)c([2H])c1[2H] ; [2H]c1c([2H])c([2H])c(-c2cc(-c3nc(-c4ccccc4)nc(-c4ccc(-c5cccc6c(-c7ccccc7)cccc56)cc4)n3)c3c(c2)oc2ccccc23)c([2H])c1[2H] ; [2H]c1c([2H])c([2H])c(-c2cc(-c3nc(-c4ccccc4)nc(-c4ccc(-c5cccc6c(-c7ccccc7)cccc56)cc4)n3)c3c(oc4ccccc43)c2[2H])c([2H])c1[2H]]

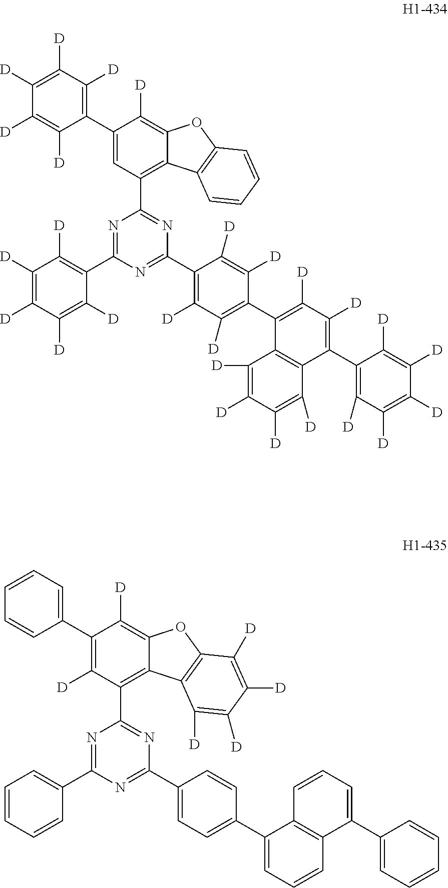 [SMILES: [2H]c1c([2H])c([2H])c(-c2nc(-c3c([2H])c([2H])c(-c4c([2H])c([2H])c(-c5c([2H])c([2H])c([2H])c([2H])c5[2H])c5c([2H])c([2H])c([2H])c([2H])c45)c([2H])c3[2H])nc(-c3cc(-c4c([2H])c([2H])c([2H])c([2H])c4[2H])c([2H])c4oc5ccccc5c34)n2)c([2H])c1[2H] ; [2H]c1c([2H])c([2H])c2c(oc3c([2H])c(-c4ccccc4)c([2H])c(-c4nc(-c5ccccc5)nc(-c5ccc(-c6cccc7c(-c8ccccc8)cccc67)cc5)n4)c32)c1[2H]]

Hereinafter, an organic electroluminescent device will be described to which the aforementioned plurality of host materials is applied.

An organic electroluminescent device according to one embodiment has a first electrode; a second electrode; and at least one organic layer interposed between the first electrode and the second electrode. According to one embodiment, the first host material including the compound represented by Formula 1 and the second host material including the compound represented by Formula 2 may be included in the same organic layer, or may be included in different organic layers, respectively.

The organic layer includes at least one light-emitting layer, and the at least one light-emitting layer may include a plurality of host materials comprising at least one first host material including the compound represented by Formula 1 and at least one second host material including the compound represented by Formula 2. According to one embodiment, the light-emitting layer may include at least one compound among Compounds H1-1 to H1-437, which are first host materials represented by Formula 1, and at least one compound among Compounds H2-1 to H2-391, H3-1 to H3-299, and H4-1 to H4-171, which are second host materials represented by Formula 2.

One of the first electrode and the second electrode may be an anode and the other may be a cathode. Wherein, the first electrode and the second electrode may each be formed as a transmissive conductive material, a transflective conductive material, or a reflective conductive material. The organic layer may further comprise at least one layer selected from a hole injection layer, a hole transport layer, a hole auxiliary layer, a light-emitting auxiliary layer, an electron transport layer, an electron injection layer, an interlayer, a hole-blocking layer, an electron-blocking layer, and an electron buffer layer, in addition to the light-emitting layer.

The organic layer may further comprise an amine-based compound and/or an azine-based compound other than the light-emitting material according to the present disclosure. Specifically, the hole injection layer, the hole transport layer, the hole auxiliary layer, the light-emitting layer, the light-emitting auxiliary layer, or the electron-blocking layer may contain the amine-based compound, e.g., an arylamine-based compound and a styrylarylamine-based compound, etc., as a hole injection material, a hole transport material, a hole auxiliary material, a light-emitting material, a light-emitting auxiliary material, or an electron-blocking material. Also, the electron transport layer, the electron injection layer, the electron buffer layer, or the hole-blocking layer may contain the azine-based compound as an electron transport material, an electron injection material, an electron buffer material, or a hole-blocking material. Also, the organic layer may further comprise at least one metal selected from the group consisting of metals of Group 1, metals of Group 2, transition metals of the 4^{th} period, transition metals of the 5^{th} period, lanthanides, and organic metals of the d-transition elements of the Periodic Table, or at least one complex compound comprising such a metal.

The plurality of host materials according to one embodiment may be used as light-emitting materials for a white organic light-emitting device. The white organic light-emitting device has been suggested to have various structures such as a parallel side-by-side arrangement method, a stacking arrangement method, or CCM (color conversion material) method, etc. according to the arrangement of R (red), G (green), YG (yellowish green), or B (blue) light-emitting units. In addition, the plurality of host materials according to one embodiment may also be applied to the organic electroluminescent device comprising a QD (quantum dot).

A hole injection layer, a hole transport layer, an electron-blocking layer, or a combination thereof can be used between the anode and the light-emitting layer. The hole injection layer may be multi-layers in order to lower the hole injection barrier (or hole injection voltage) from the anode to the hole transport layer or the electron-blocking layer, wherein each of the multi-layers may use two compounds simultaneously. Also, the hole injection layer may be doped with a p-dopant. Also, the electron-blocking layer may be placed between the hole transport layer (or hole injection layer) and the light-emitting layer, and can confine the excitons within the light-emitting layer by blocking the overflow of electrons from the light-emitting layer to prevent light-emitting leakage. The hole transport layer or the electron-blocking layer may be multi-layers, wherein each layer may use a plurality of compounds.

An electron buffer layer, a hole-blocking layer, an electron transport layer, an electron injection layer, or a combination thereof can be used between the light-emitting layer and the cathode. The electron buffer layer may be multi-layers in order to control the injection of the electron and improve the interfacial properties between the light-emitting layer and the electron injection layer, wherein each of the multi-layers may use two compounds simultaneously. The hole-blocking layer or the electron transport layer may also be multi-layers, wherein each layer may use a plurality of compounds. Also, the electron injection layer may be doped with an n-dopant.

The light-emitting auxiliary layer may be placed between the anode and the light-emitting layer, or between the cathode and the light-emitting layer. When the light-emitting auxiliary layer is placed between the anode and the light-emitting layer, it can be used for promoting the hole injection and/or the hole transport, or for preventing the overflow of electrons. When the light-emitting auxiliary layer is placed between the cathode and the light-emitting layer, it can be used for promoting the electron injection and/or the electron transport, or for preventing the overflow of holes. In addition, the hole auxiliary layer may be placed between the hole transport layer (or hole injection layer) and the light-emitting layer, and may be effective to promote or block the hole transport rate (or the hole injection rate), thereby enabling the charge balance to be controlled. When an organic electroluminescent device includes two or more hole transport layers, the hole transport layer, which is further included, may be used as the hole auxiliary layer or the electron-blocking layer. The light-emitting auxiliary layer, the hole auxiliary layer, or the electron-blocking layer may have an effect of improving the efficiency and/or the lifespan of the organic electroluminescent device.

In the organic electroluminescent device of the present disclosure, preferably, at least one layer (hereinafter, “a surface layer”) selected from a chalcogenide layer, a halogenated metal layer, and a metal oxide layer may be placed on an inner surface(s) of one or both of a pair of electrodes. Specifically, a chalcogenide (including oxides) layer of silicon and aluminum is preferably placed on an anode surface of an electroluminescent medium layer, and a halogenated metal layer or a metal oxide layer is preferably placed on a cathode surface of an electroluminescent medium layer. The operation stability for the organic electroluminescent device may be obtained by the surface layer. Preferably, the chalcogenide includes SiOₓ (1≤X≤2), AlOₓ (1≤X≤1.5), SiON, SiAlON, etc.; the halogenated metal includes LiF, MgF₂, CaF₂, a rare earth metal fluoride, etc.; and the metal oxide includes Cs₂O, Li₂O, MgO, SrO, BaO, CaO, etc.

The organic electroluminescent device according to one embodiment of the present disclosure may be an organic electroluminescent device having a tandem structure. In the case of a tandem organic electroluminescent device according to one embodiment, a single light-emitting unit (light-emitting unit) may be formed in a structure in which two or more units are connected by a charge generation layer. The organic electroluminescent device may include a plurality of two or more light-emitting units, for example, a plurality of three or more light-emitting units, having first and second electrodes opposed to each other on a substrate and a light-emitting layer that is stacked between the first and second electrodes and emits light in a specific wavelength range, wherein each of the light-emitting units may include a hole transport band, a light-emitting layer, and an electron transport band, and the hole transport band may include a hole injection layer and a hole transport layer, and the electron transport zone may include an electron transport layer and an electron injection layer. According to one embodiment, three or more light-emitting layers may be included in the light-emitting unit. A plurality of light-emitting units may emit the same color or different colors. Additionally, one light-emitting unit may include one or more light-emitting layers, and the plurality of light-emitting layers may be light-emitting layers of the same or different colors. This may include one or more charge generation layers located between each light-emitting unit. The charge generation layer refers to the layer in which holes and electrons are generated when voltage is applied. When there are three or more light-emitting units, a charge generation layer may be located between each light-emitting unit. At this time, the plurality of charge generation layers may be the same as or different from each other. By disposing the charge generation layer between light-emitting units, current efficiency is increased in each light-emitting unit, and charges can be smoothly distributed. Specifically, the charge generation layer is provided between two adjacent stacks and can serve to drive a tandem organic electroluminescent device using only a pair of anode and cathode without a separate internal electrode located between the stacks.

An organic electroluminescent device according to one embodiment may have two or more organic layers, and may further include one or more charge generation layers, wherein the charge generation layers may be located between each of the organic layers, and when two or more organic layers are included, each of the charge generation layers may be the same or different. Since the charge generation layers are located between the organic layers, the organic electroluminescent device may be driven by only a pair of an anode and a cathode without a separate internal electrode located between the organic layers.

The charge generation layer may be composed of an N-type charge generation layer and a P-type charge generation layer. The N-type charge generation layer may be doped with an alkali metal, an alkaline earth metal, or a compound of an alkali metal and an alkaline earth metal. The alkali metal may include one selected from the group consisting of Li, Na, K, Rb, Cs, Fr, Yb, and combinations thereof, and the alkaline earth metal may include one selected from the group consisting of Be, Mg, Ca, Sr, Ba, Ra, and combinations thereof.

In addition, in the organic electroluminescent device of the present disclosure, a mixed region of an electron transport compound and a reductive dopant, or a mixed region of a hole transport compound and an oxidative dopant may be placed on at least one surface of a pair of electrodes. In this case, the electron transport compound is reduced to an anion, and thus it becomes easier to inject and transport electrons from the mixed region to an electroluminescent medium. Furthermore, the hole transport compound is oxidized to a cation, and thus it becomes easier to inject and transport holes from the mixed region to the electroluminescent medium. Preferably, the oxidative dopant includes various Lewis acids and acceptor compounds, and the reductive dopant includes alkali metals, alkali metal compounds, alkaline earth metals, rare earth metals, and mixtures thereof. Also, a reductive dopant layer may be employed as a charge generation layer to prepare an organic electroluminescent device which has two or more light-emitting layers and emits white light.

The light-emitting layer of an organic electroluminescent device according to one embodiment may be a single layer in which light is emitted, or may be a plurality of layers in which two or more layers are stacked. The light-emitting layer may further include one or more dopants, and the doping concentration of the dopant compound with respect to the host compound of the light-emitting layer may be less than 20 wt %, preferably less than 10 wt %.

The dopant comprised in the organic electroluminescent device of the present disclosure may be at least one phosphorescent or fluorescent dopant, preferably a phosphorescent dopant. The phosphorescent dopant material applied to the organic electroluminescent device of the present disclosure is not particularly limited, but preferably may be a metallated complex compound(s) of a metal atom(s) selected from iridium (Ir), osmium (Os), copper (Cu), and platinum (Pt), more preferably an ortho-metallated complex compound(s) of a metal atom(s) selected from iridium (Ir), osmium (Os), copper (Cu), and platinum (Pt), and even more preferably ortho-metallated iridium complex compound(s).

The dopant comprised in the organic electroluminescent device of the present disclosure may use the compound represented by the following Formula 101, but is not limited thereto.

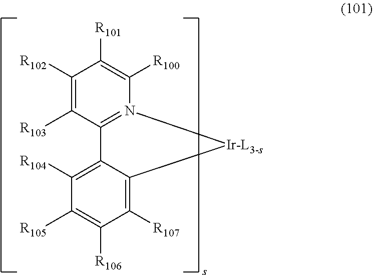

In Formula 101,
L is any one selected from the following Structures 1 to 3;

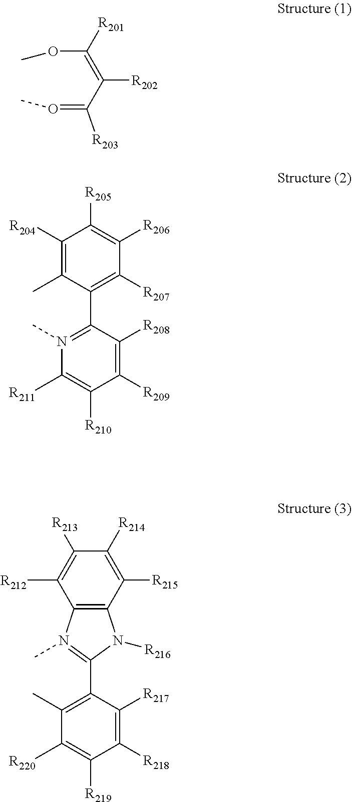

R₁₀₀ to R₁₀₃ each independently represent hydrogen, deuterium, a halogen, (C₁-C₃₀)alkyl unsubstituted or substituted with deuterium and/or halogen, a substituted or unsubstituted (C₃-C₃₀)cycloalkyl, a substituted or unsubstituted (C₆-C₃₀)aryl, cyano, a substituted or unsubstituted (3- to 30-membered)heteroaryl, or a substituted or unsubstituted (C₁-C₃₀)alkoxy; or may be linked to the adjacent substituents to form a ring(s), for example, to form a ring(s) with a pyridine, e.g., a substituted or unsubstituted quinoline, a substituted or unsubstituted benzofuropyridine, a substituted or unsubstituted benzothienopyridine, a substituted or unsubstituted indenopyridine, a substituted or unsubstituted benzofuroquinoline, a substituted or unsubstituted benzothienoquinoline, or a substituted or unsubstituted indenoquinoline;
R₁₀₄ to R₁₀₇ each independently represent hydrogen, deuterium, a halogen, (C₁-C₃₀)alkyl unsubstituted or substituted with deuterium and/or halogen, a substituted or unsubstituted (C₃-C₃₀)cycloalkyl, a substituted or unsubstituted (C₆-C₃₀)aryl, a substituted or unsubstituted (3- to 30-membered)heteroaryl, cyano, or a substituted or unsubstituted (C₁-C₃₀)alkoxy; or may be linked to the adjacent substituents to form a substituted or unsubstituted ring(s), for example, to form a substituted or unsubstituted ring(s) with a benzene, e.g., a substituted or unsubstituted naphthalene, a substituted or unsubstituted fluorene, a substituted or unsubstituted dibenzothiophene, a substituted or unsubstituted dibenzofuran, a substituted or unsubstituted indenopyridine, a substituted or unsubstituted benzofuropyridine, or a substituted or unsubstituted benzothienopyridine;
R₂₀₁ to R₂₂₀ each independently represent hydrogen, deuterium, a halogen, (C₁-C₃₀)alkyl unsubstituted or substituted with deuterium and/or halogen, a substituted or unsubstituted (C₃-C₃₀)cycloalkyl, or a substituted or unsubstituted (C₆-C₃₀)aryl; or may be linked to the adjacent substituents to form a substituted or unsubstituted ring(s); and
s represents an integer of 1 to 3.

Specifically, the specific examples of the dopant compound include the following, but are not limited thereto.

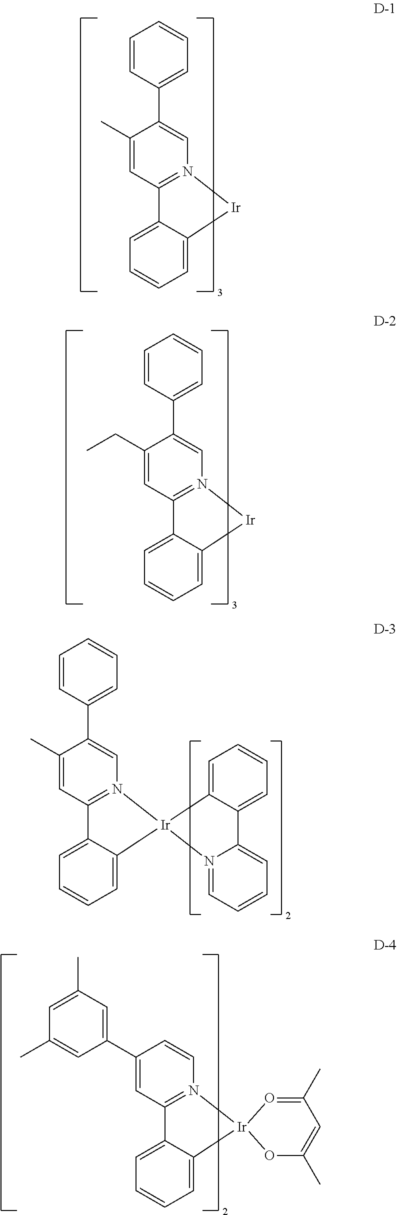

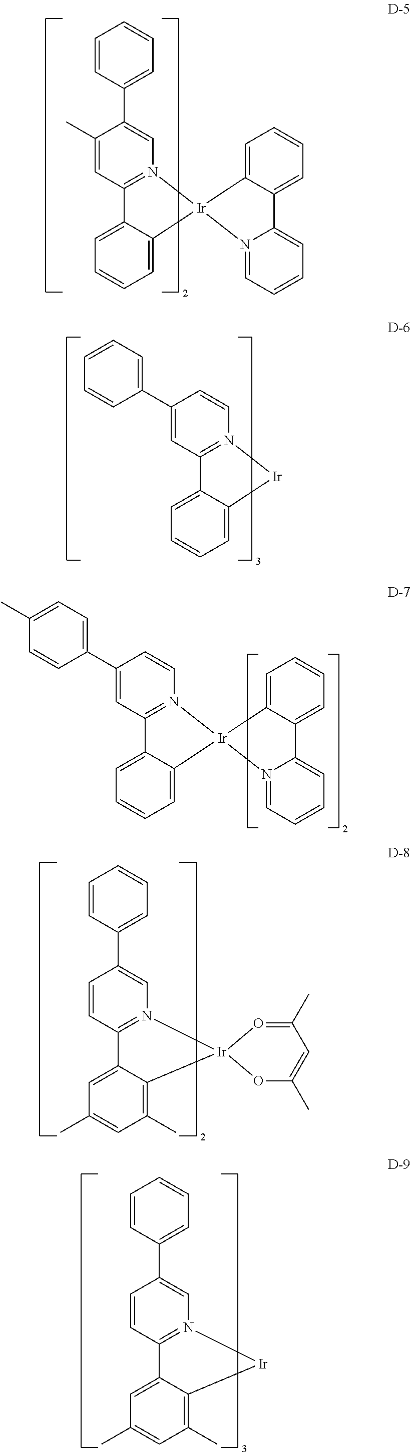

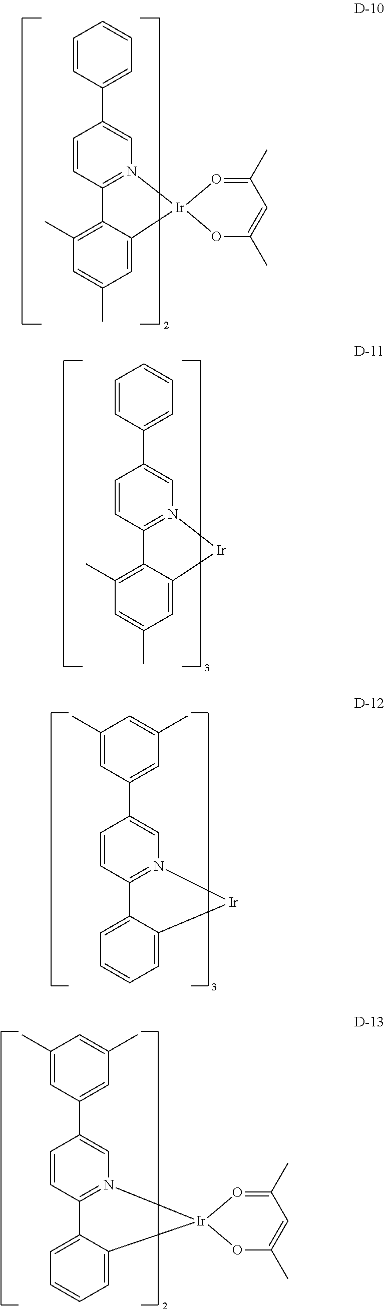

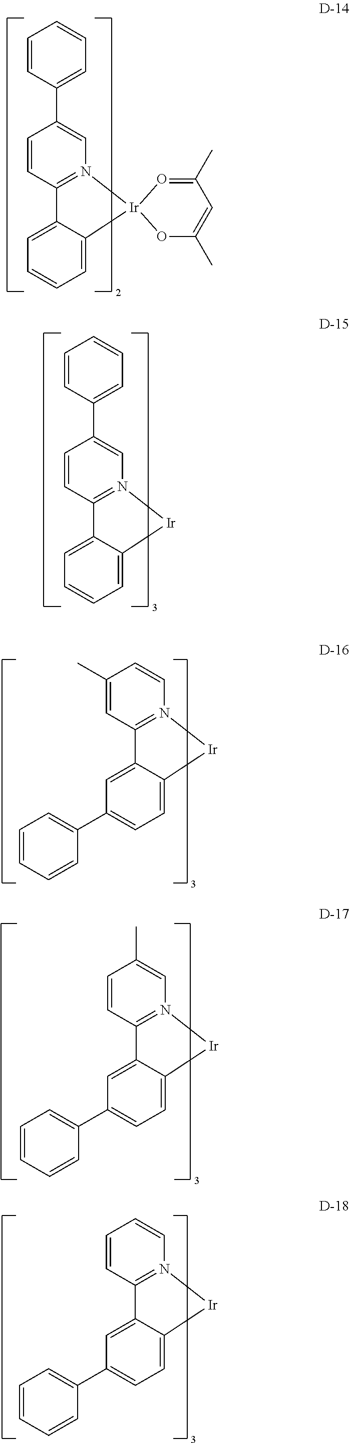

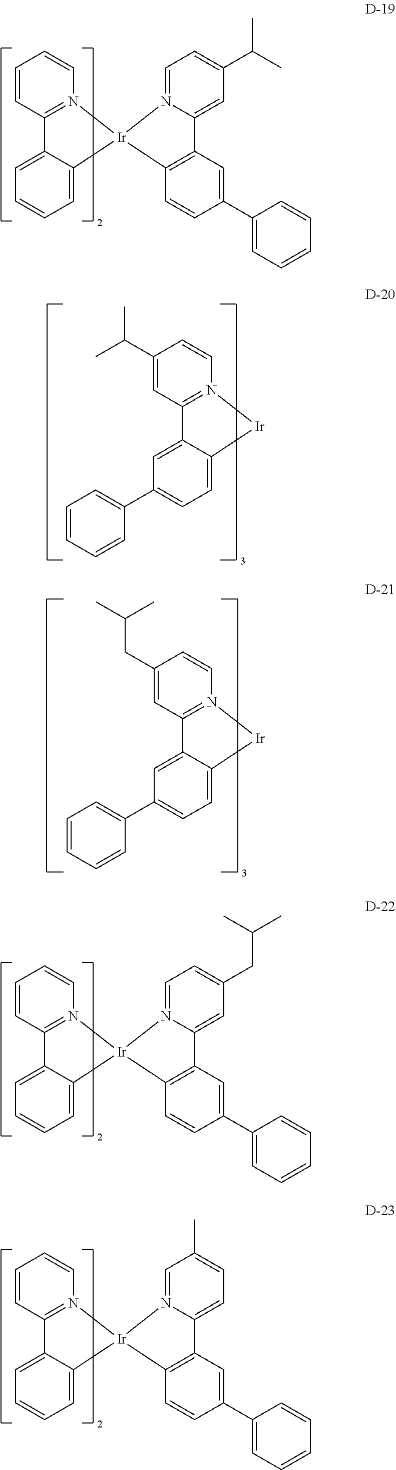

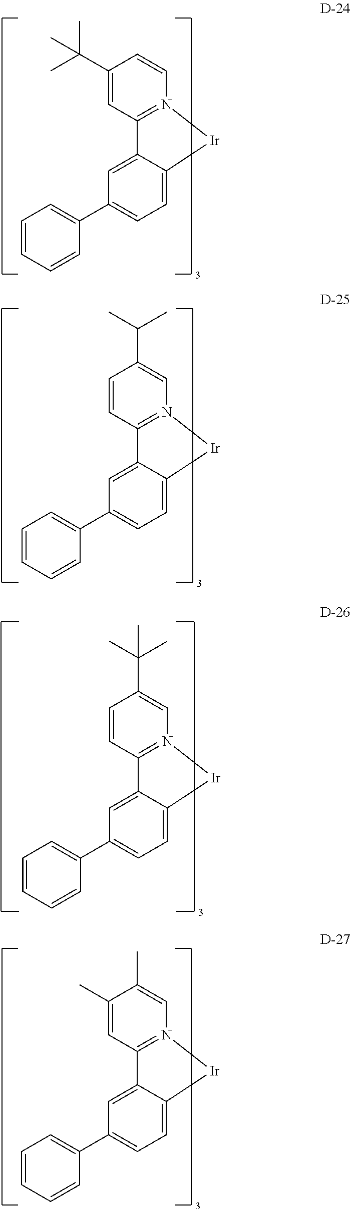

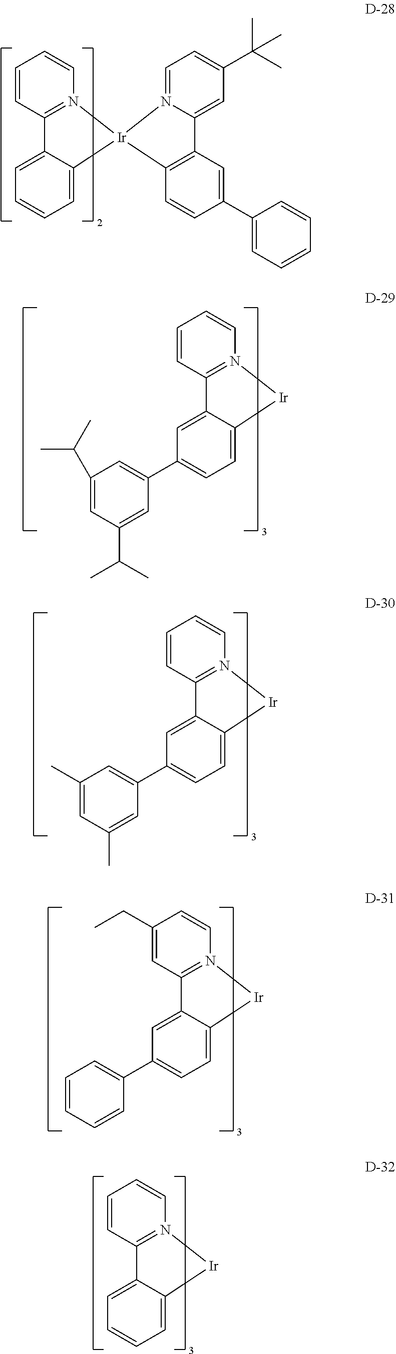

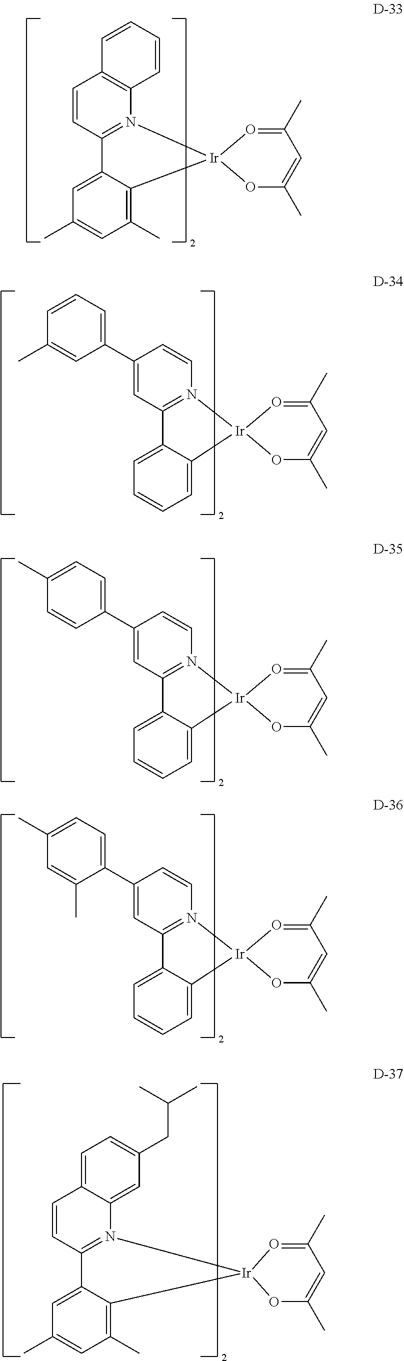

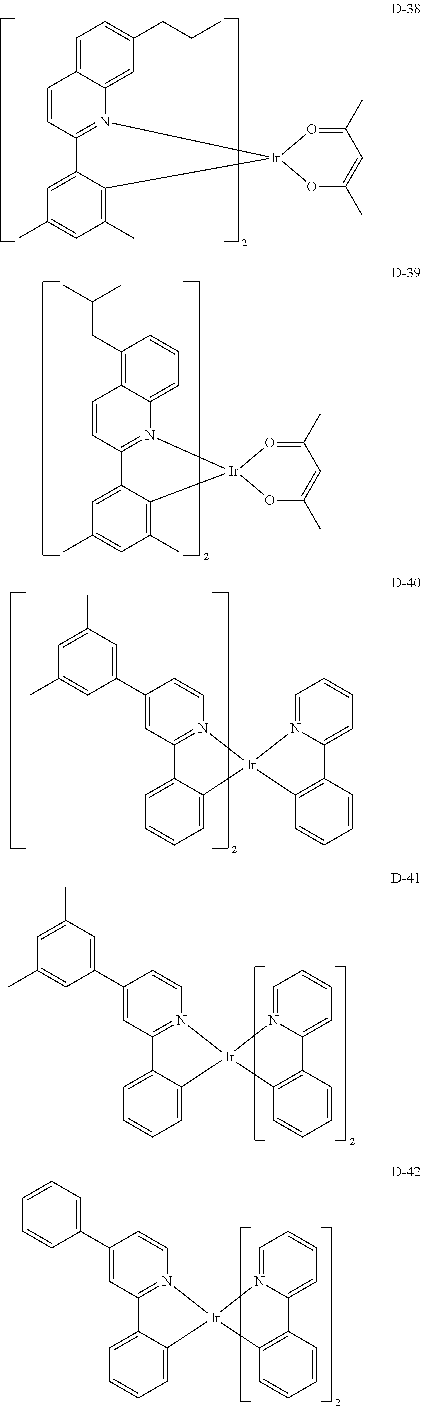

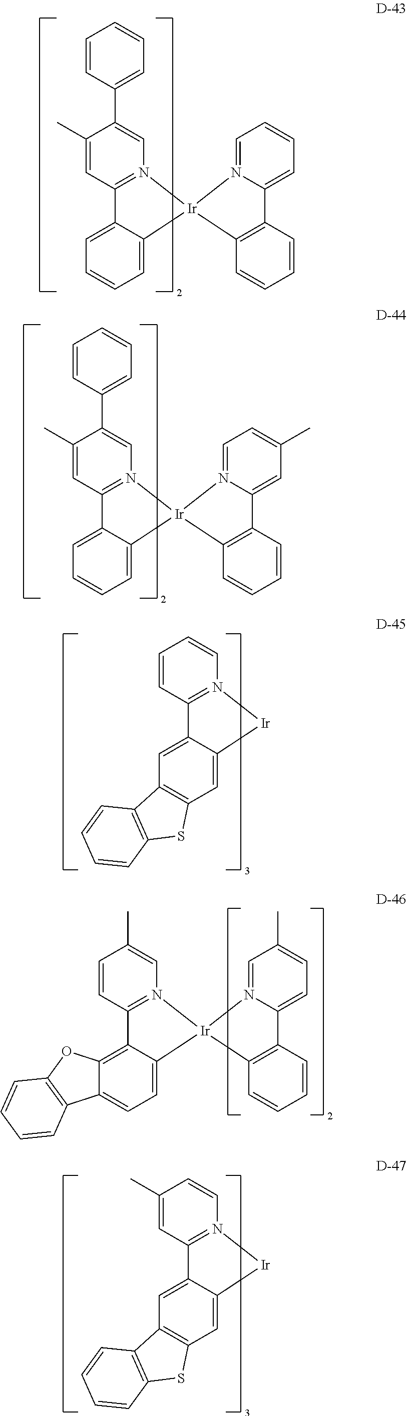

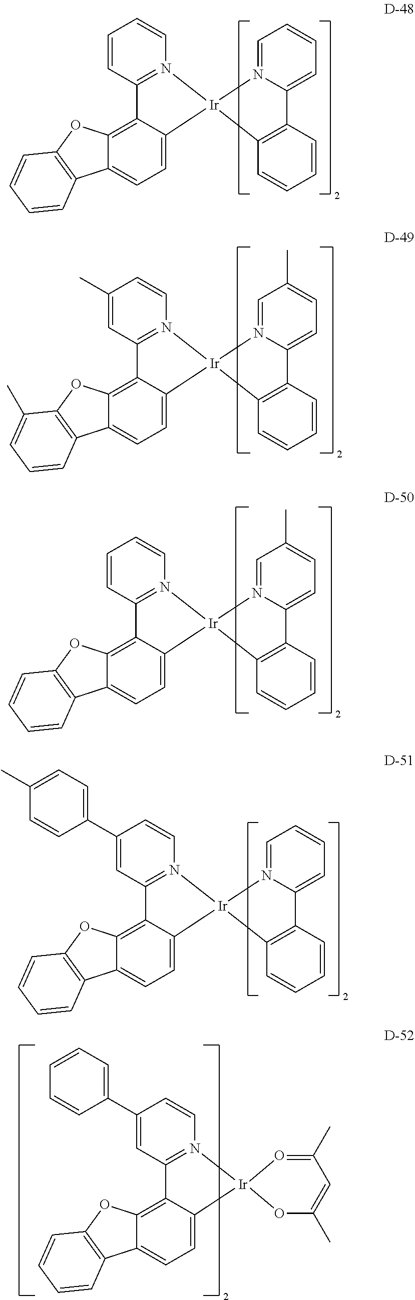

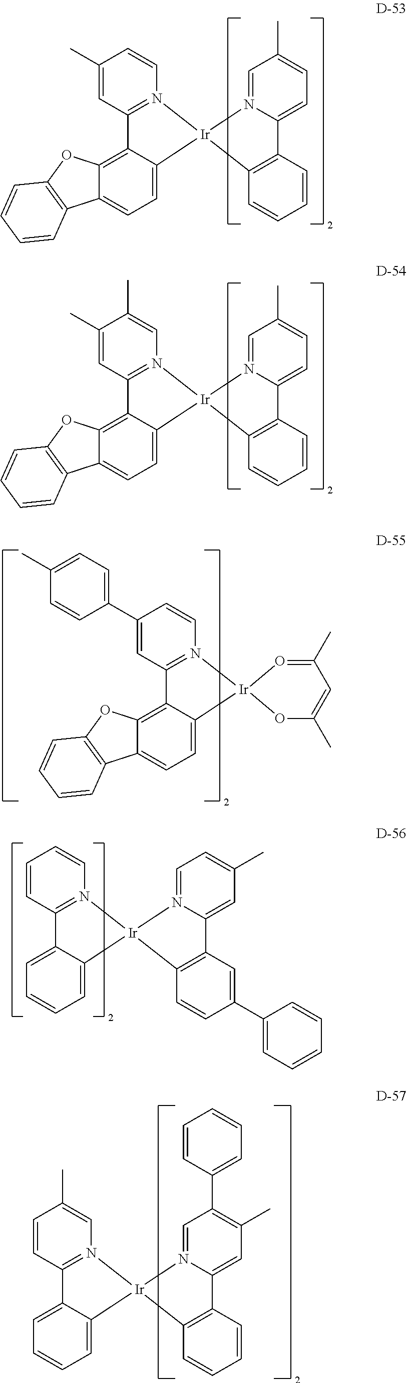

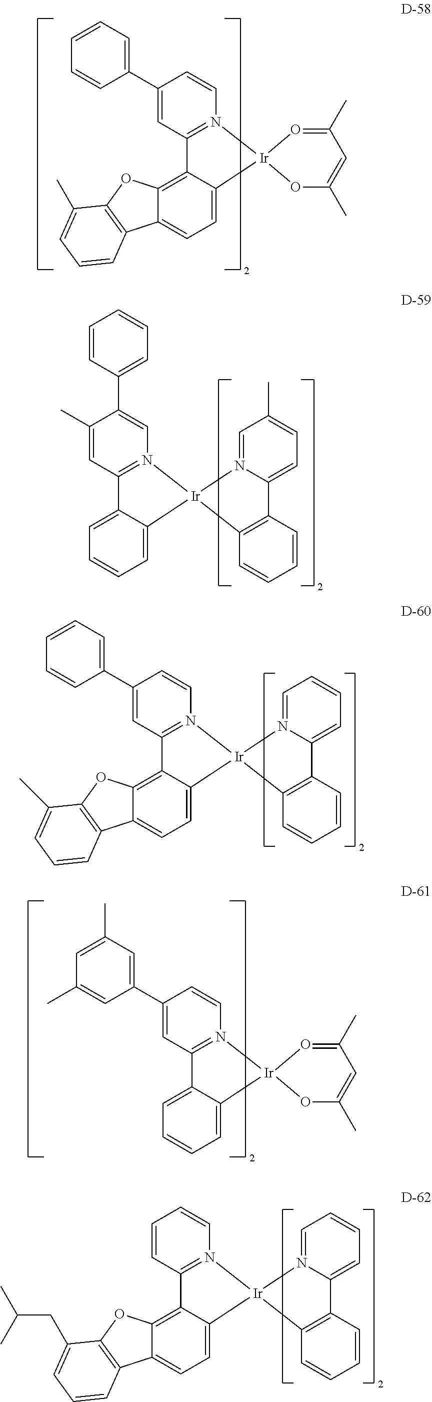

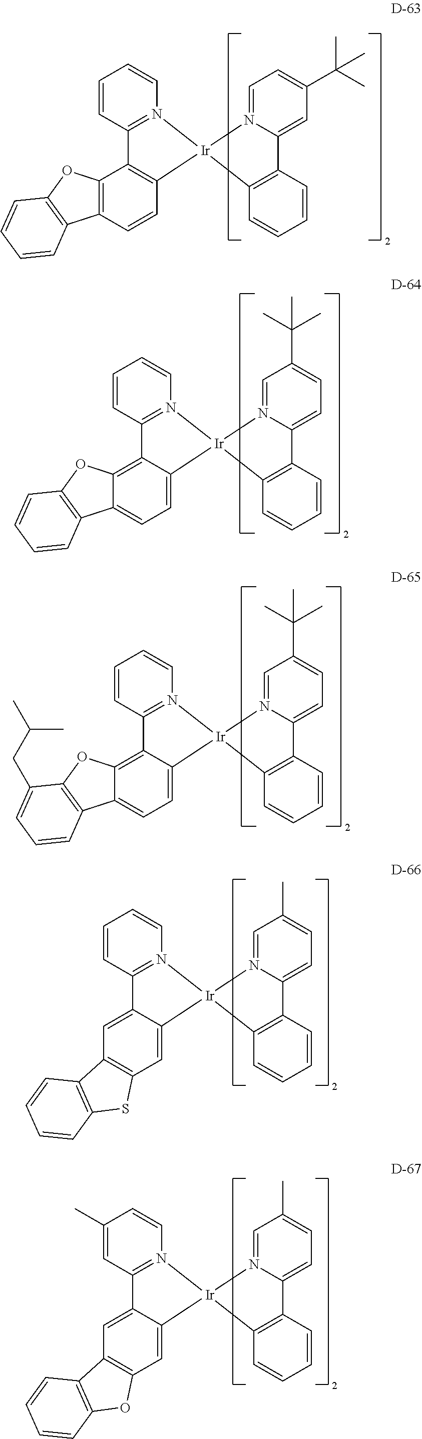

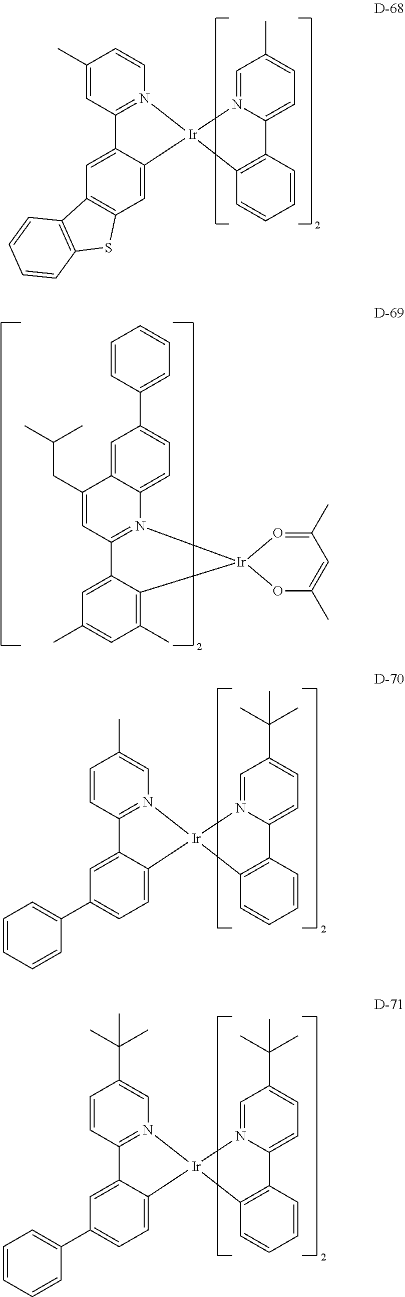

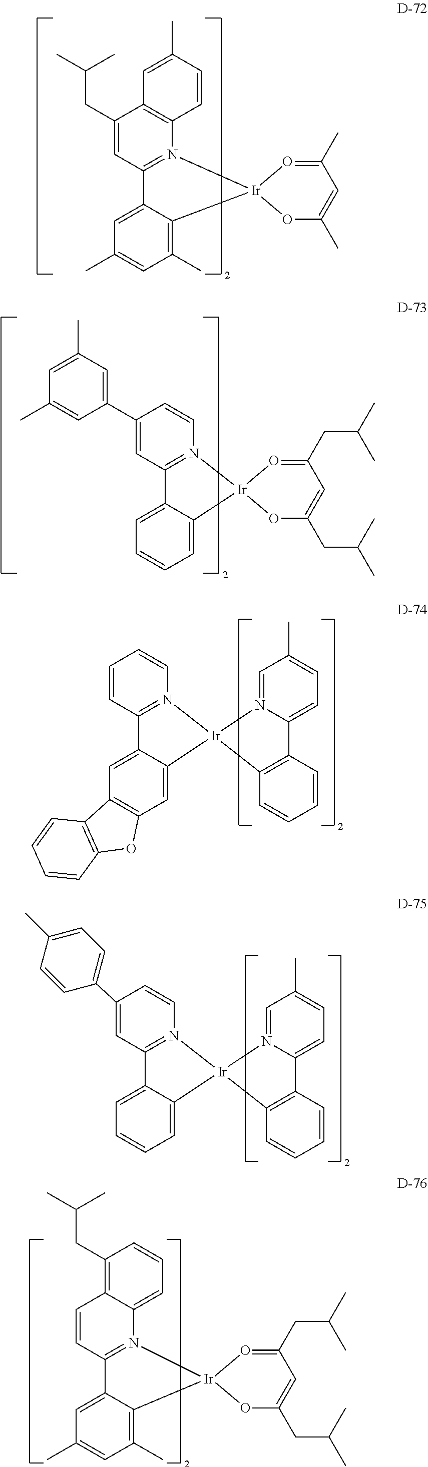

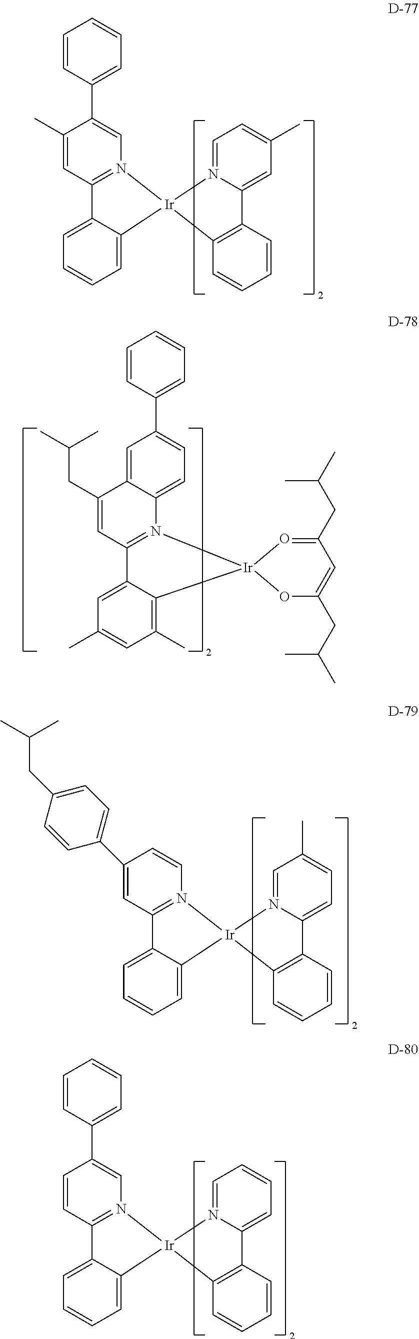

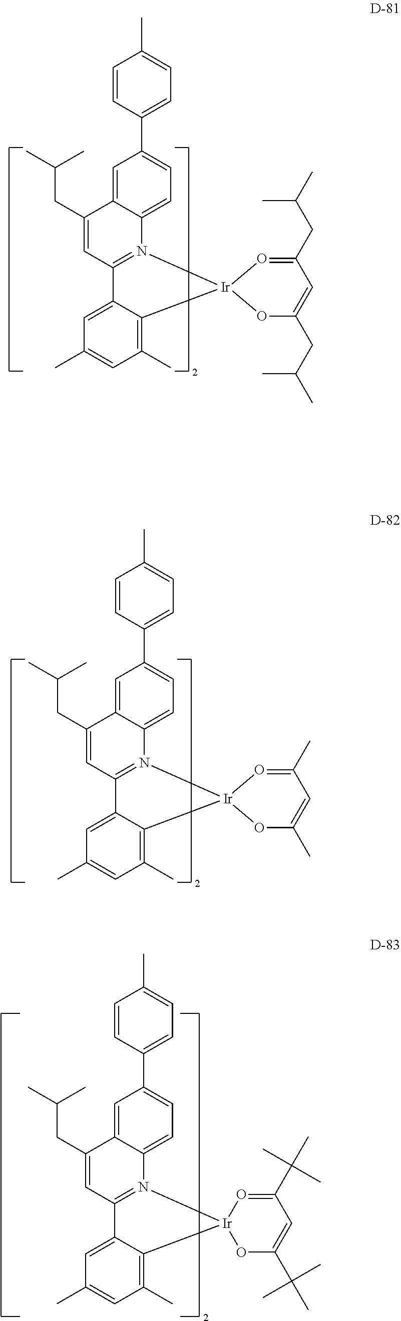

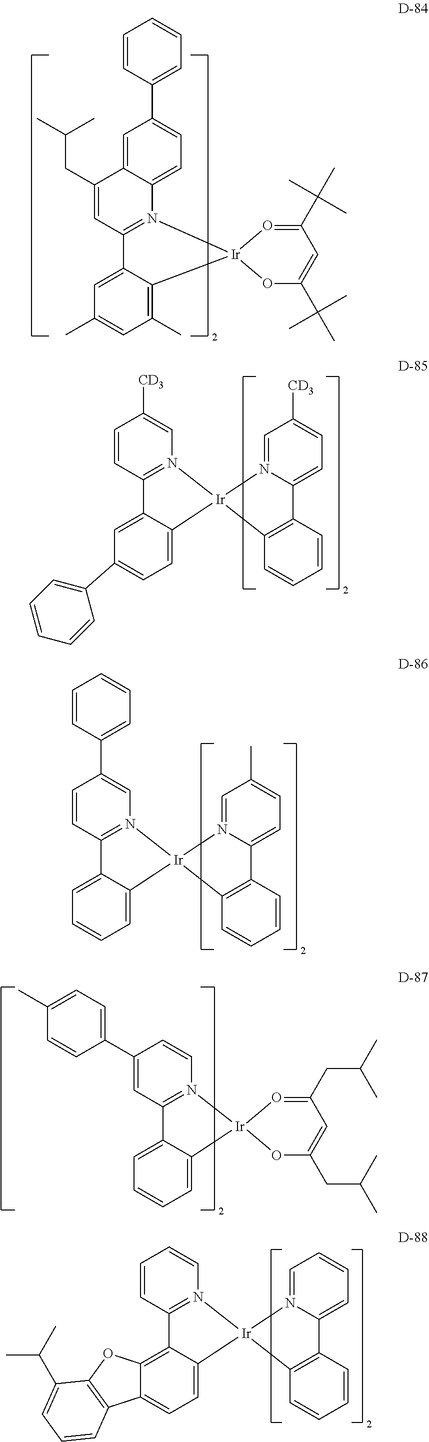

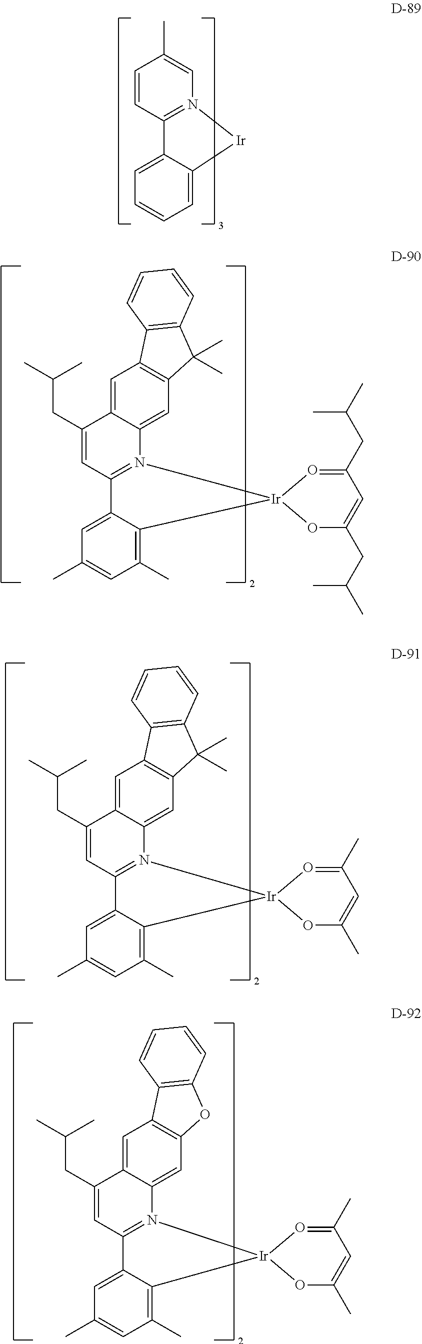

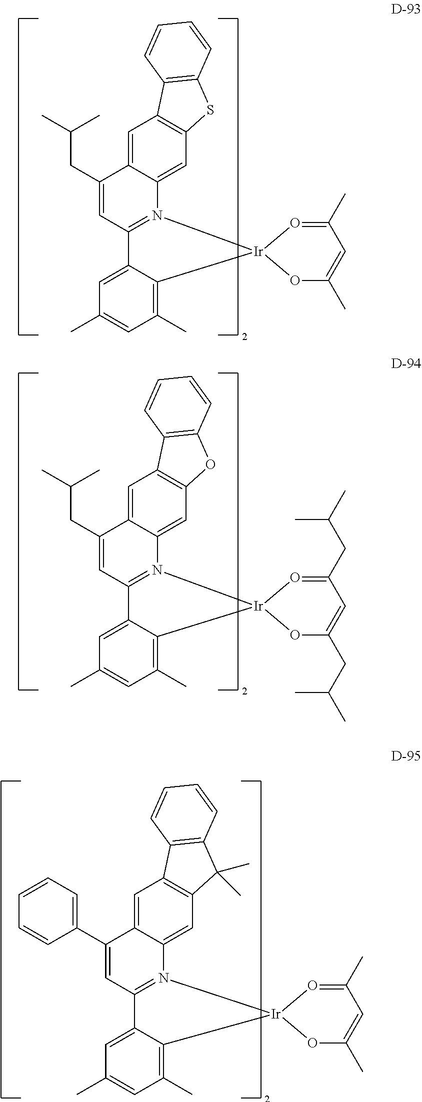

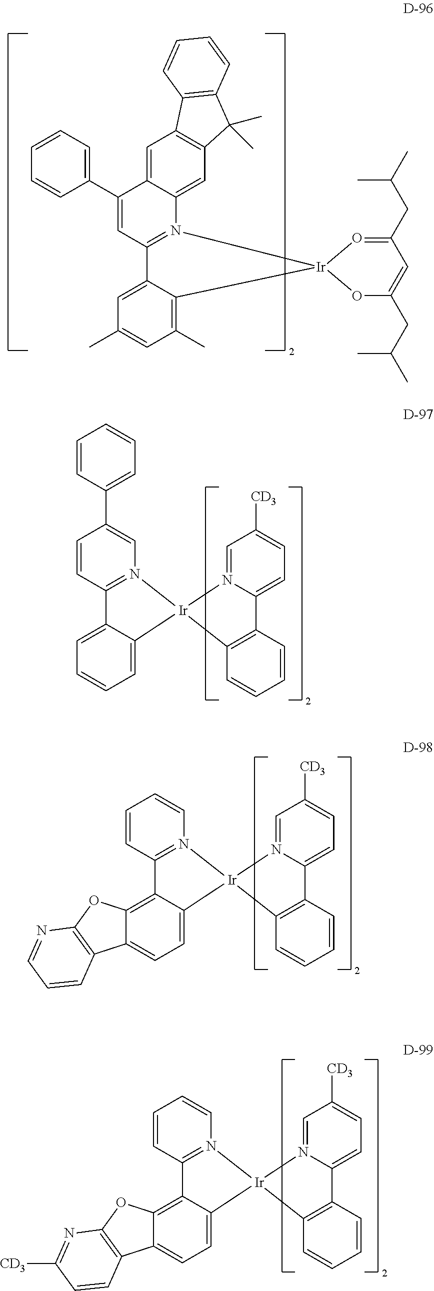

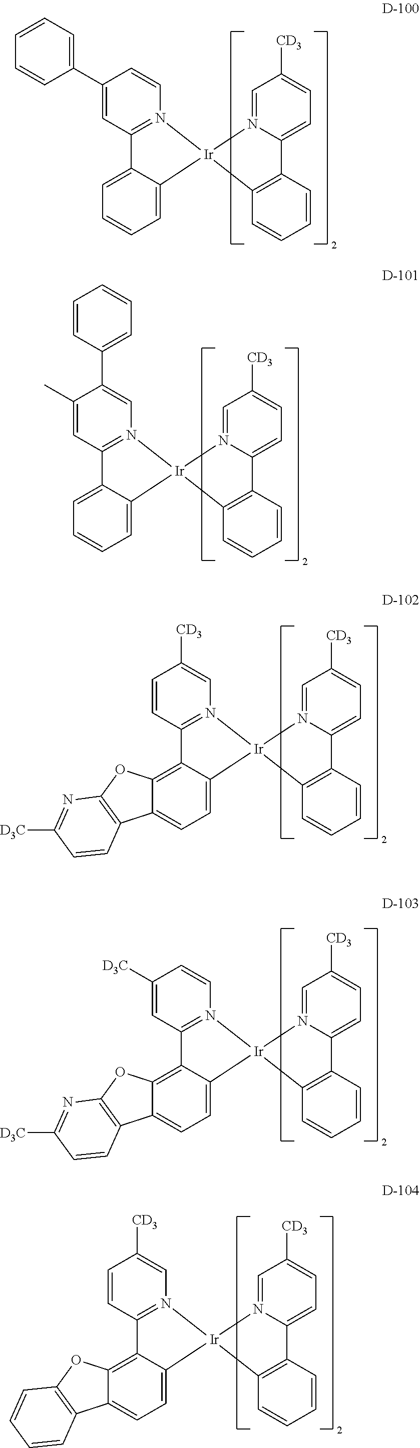

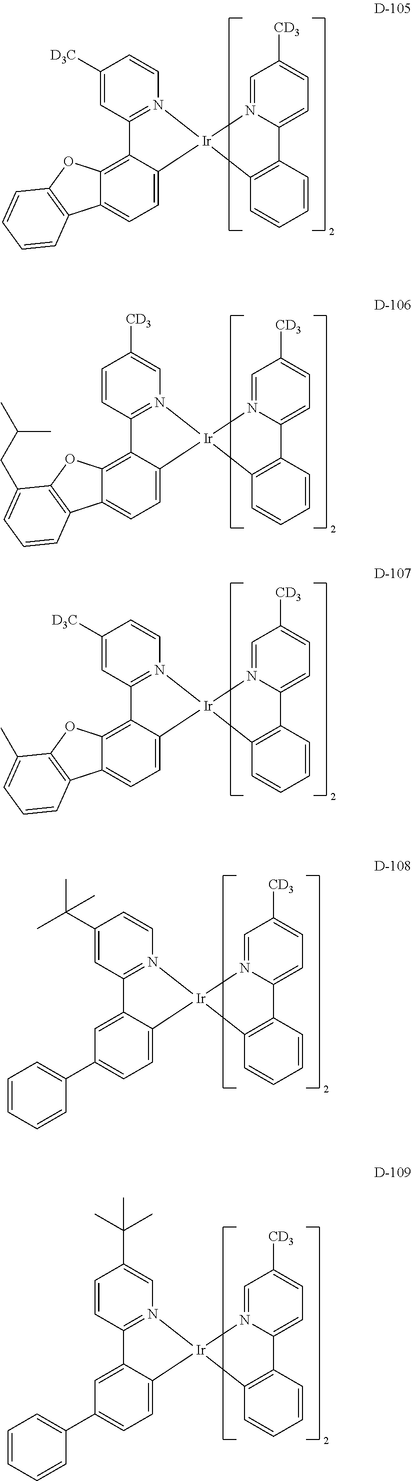

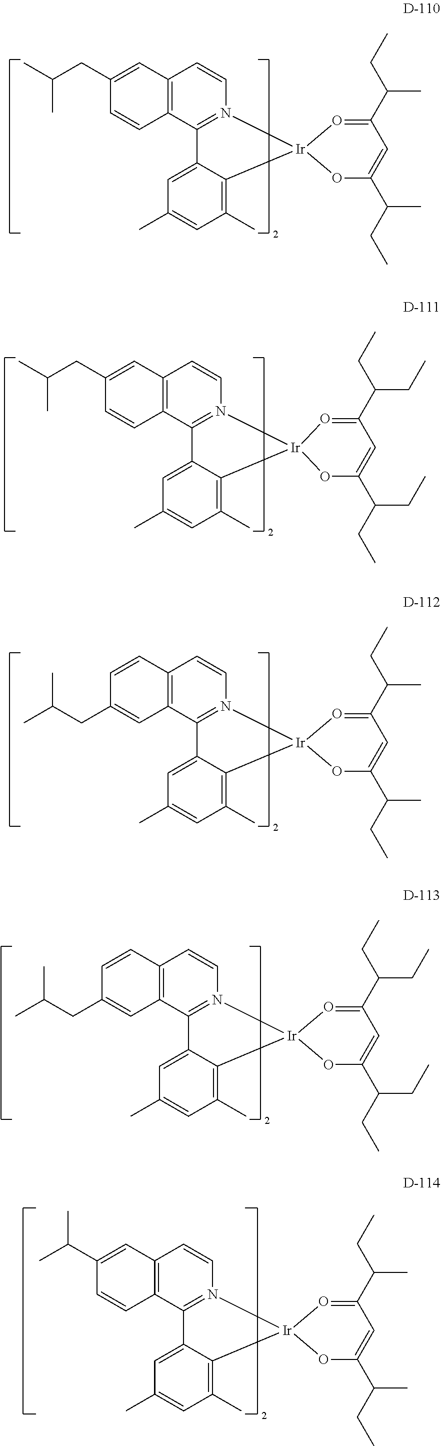

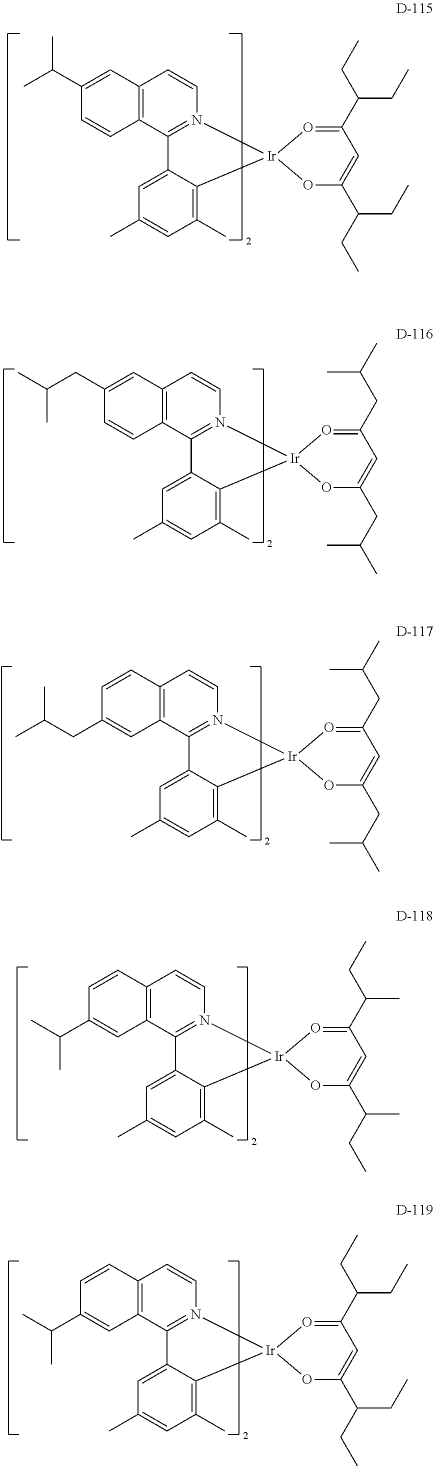

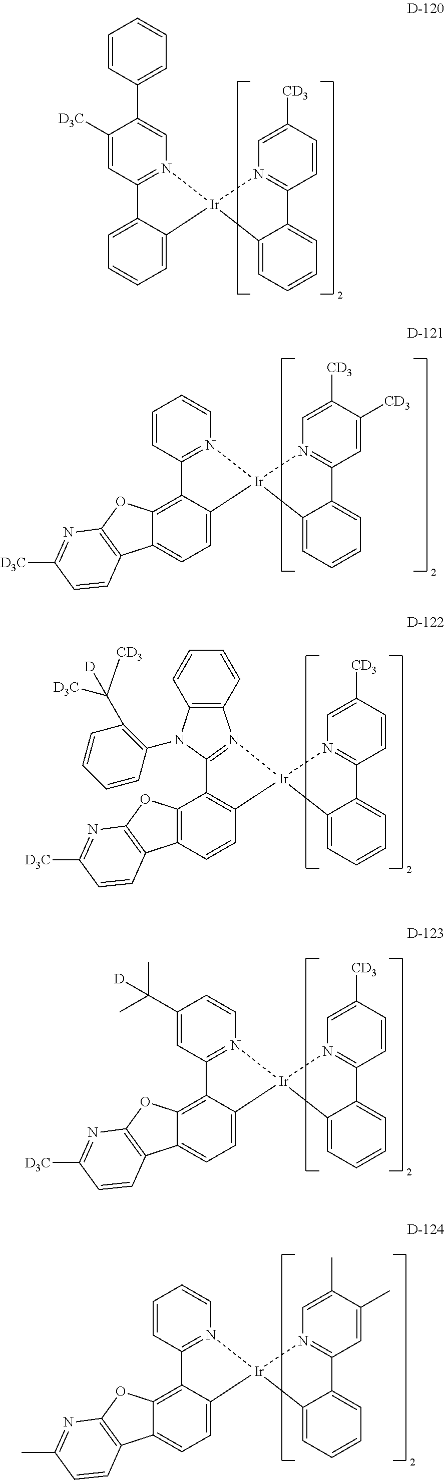

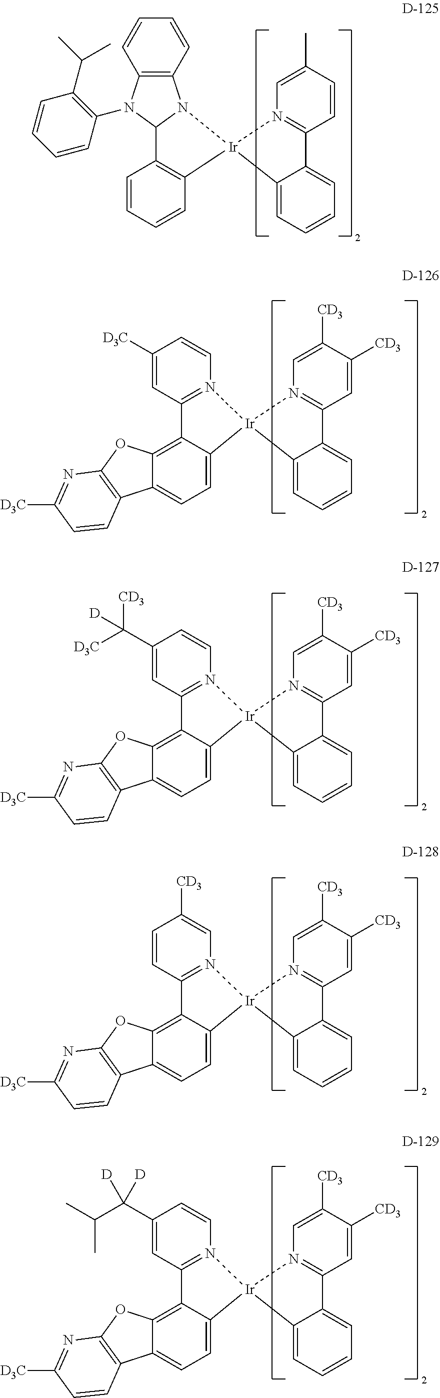

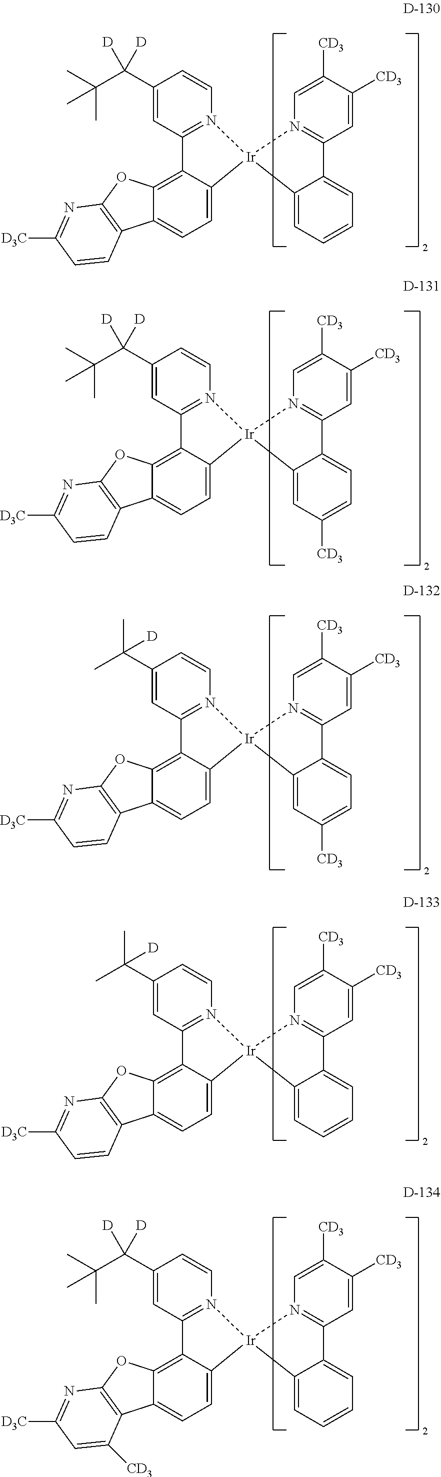

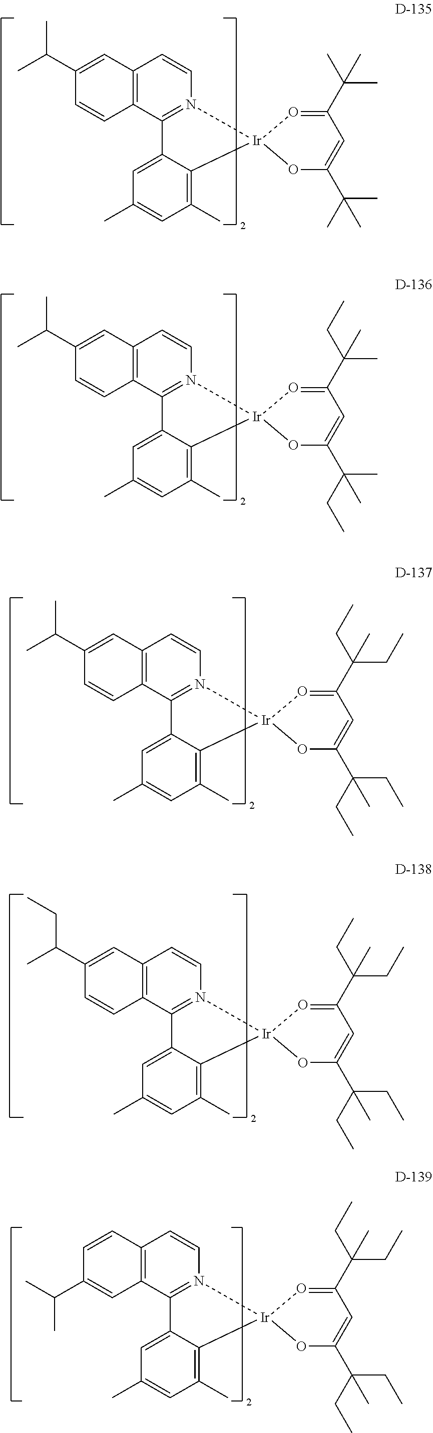

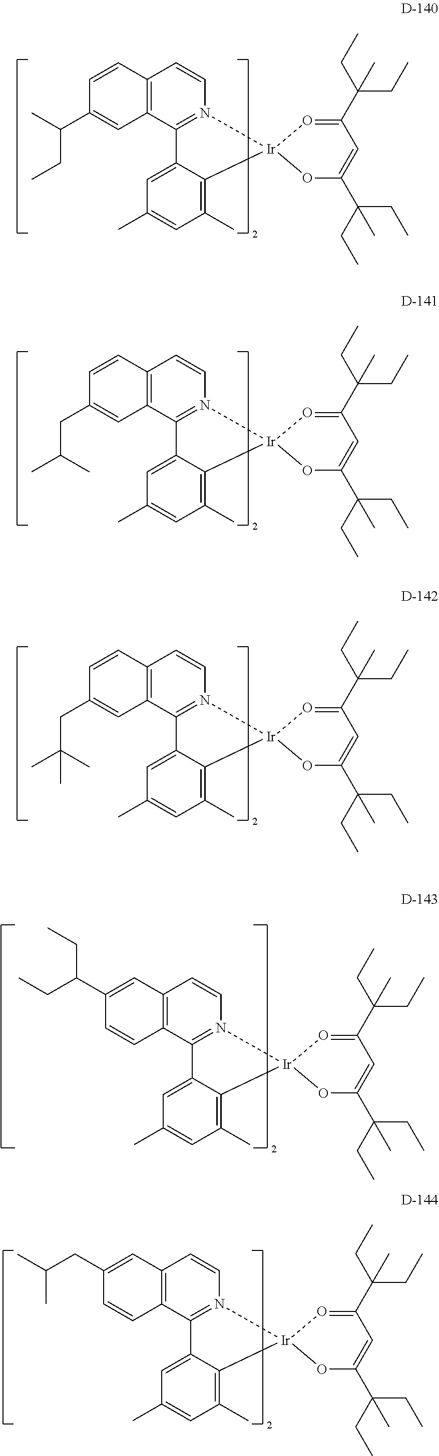

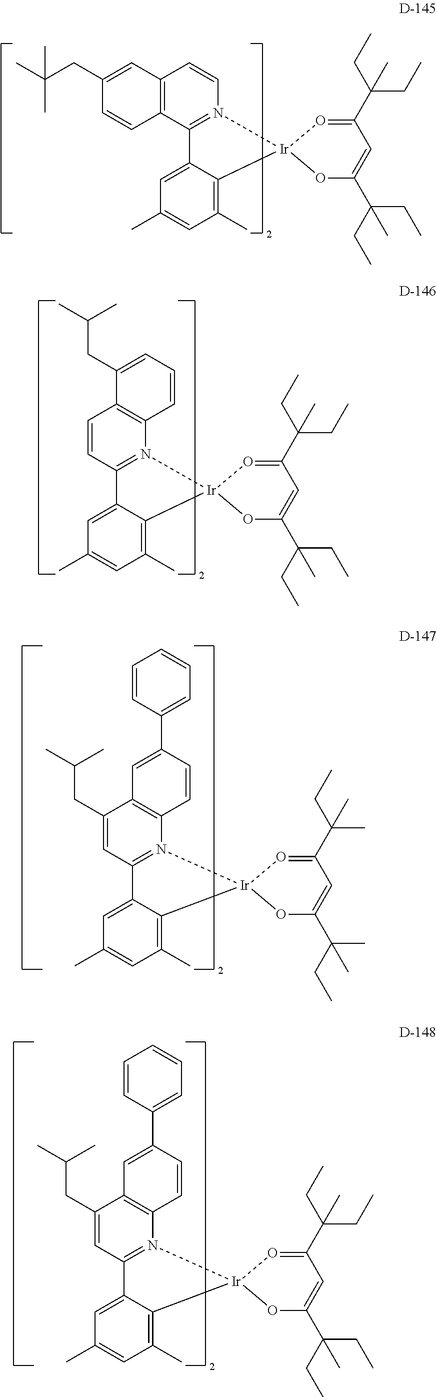

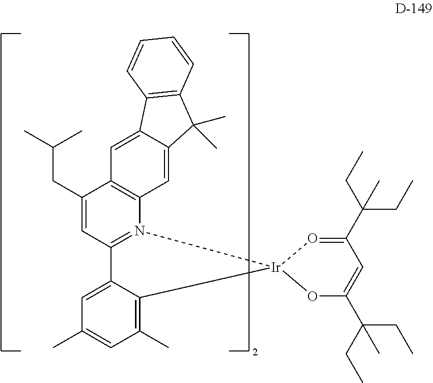

In order to form each layer of the organic electroluminescent device of the present disclosure, dry film-forming methods such as vacuum evaporation, sputtering, plasma, ion plating methods, etc. or wet film-forming methods such as ink jet printing, nozzle printing, slot coating, spin coating, dip coating, flow coating methods, etc. can be used. When using a wet film-forming method, a thin film may be formed by dissolving or diffusing materials forming each layer into any suitable solvent such as ethanol, chloroform, tetrahydrofuran, dioxane, etc. The solvent may be any solvent where the materials forming each layer can be dissolved or diffused, and where there are no problems in film-formation capability.

According to one embodiment, when the first host material and the second host material are present in the same layer or different layers in the organic electroluminescent device, the two host materials may be individually deposited. For example, the second host material may be deposited after depositing the first host material.

According to one embodiment, when each layer of the organic electroluminescent device is formed, the film may be formed by the above-described method, and the film may be formed in a co-deposition process, a mixed deposition process, and/or a process using the co-deposition process and the mixed deposition process together. For example, the co-deposition may be a method of depositing two or more isomeric materials by putting the isomeric materials into each individual vaporization source, for example, a crucible source, and simultaneously applying current to two cells to evaporate the materials. In addition, for example, the mixed deposition may be a method of mixing two or more isomeric materials in one evaporation source, for example, a crucible source before deposition, and then evaporating the materials by applying current to one cell. In addition, for example, a process using co-deposition and mixed deposition may be a process of mixing the first host material and the second host material in one evaporation source, for example, a crucible source, putting another material in another evaporation source, for example, a crucible source, and then applying current to two cells at the same time to evaporate and deposit each material. When the film is formed by using the mixed deposition and/or the process using the co-deposition and the mixed deposition together, the number of evaporation sources used may be reduced.

According to one embodiment, the present disclosure may provide a compound obtained by depositing an organic layer in a manufacturing process of an organic electroluminescent device, and then recovering and purifying a material of the organic layer attached to deposition equipment. The recovered compound may be subjected to a purification and/or recrystallization process, and the purity of the purified and/or recrystallized compound obtained therefrom may be 99.9% or higher.

According to one embodiment, the present disclosure provides a method for recovering a plurality of host materials, comprising: depositing a plurality of host materials including at least one first host material including the compound represented by Formula 1 and at least one second host material including the compound represented by Formula 2; recovering the plurality of host materials attached to a deposition equipment; and purifying and/or recrystallizing the recovered plurality of host materials to obtain the plurality of host materials with a purity of 99.9% or higher.

According to one embodiment, the present disclosure can provide a display device such as a display device for smartphones, tablets, notebooks, PCs, TVs, or vehicles, or a lighting device such as an outdoor or indoor lighting device by using a plurality of host materials including the compound represented by Formula 1 and the compound represented by Formula 2.

Hereinafter, the preparation method of the compound according to the present disclosure and physical properties thereof will be explained with reference to the synthesis methods of the representative compounds or intermediate compounds of the present disclosure.

### [Example 1] Preparation of Compound H1-72

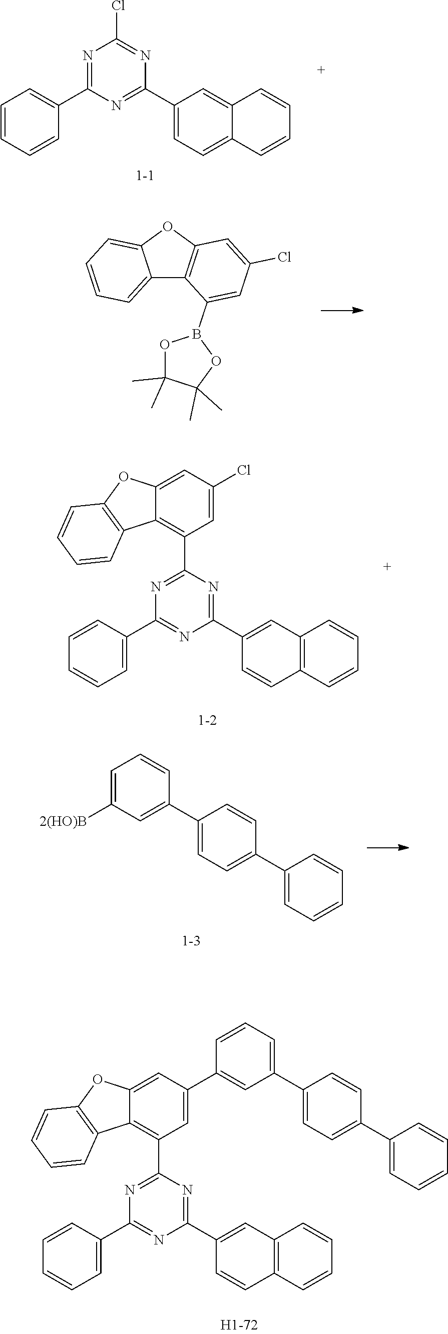

### 1. Synthesis of Compound 1-2

Compound 1-1 (15 g, 47 mmol), 2-(3-chlorodibenzo[b,d]furan-1-yl)-4,4,5,5-tetramethyl-1,3,2-dioxaborolane (17 g, 51 mmol), tetrakistriphenylphosphine (2.7 g, 2.3 mmol), and calcium carbonate (13 g, 94 mmol) were added to a flask, and 141 mL of toluene, 47 mL of ethanol, and 47 mL of distilled water were added thereto, and these were then stirred under reflux at 120° C. for 5 hours. After completion of the reaction, the mixture was cooled to room temperature, the organic layer was separated, the remaining moisture was removed with magnesium sulfate, and the solvent was distilled under reduced pressure. Thereafter, the residue was separated using column chromatography to obtain Compound 1-2 (14 g, yield: 61%).

### 2. Synthesis of Compound H1-72

Compound 1-2 (7 g, 14.4 mmol), compound 1-3 (4.36 g, 15.9 mmol), tris(dibenzylideneacetone)dipalladium(0) (1.32 g, 1.4 mmol), dicyclohexyl[2′,6′-dimethoxy[1,1′-biphenyl]-2-yl]phosphane (“SPhos”) (1.2 g, 2.8 mmol), potassium triphosphate (7.7 g, 3.6 mmol), and 100 mL of xylene were added to a flask and dissolved, and these were then stirred under reflux at 160° C. for 3 hours. After completion of the reaction, the mixture was cooled to room temperature, the organic layer was separated, and the remaining moisture was removed with magnesium sulfate. The residue was then separated using column chromatography to obtain Compound H1-72 (2.3 g, yield: 23%).

| | MW | M.P. |
|---|---|---|
| H1-72 | 677.79 | 244.8° C. |

### [Example 2] Preparation of Compound H1-85

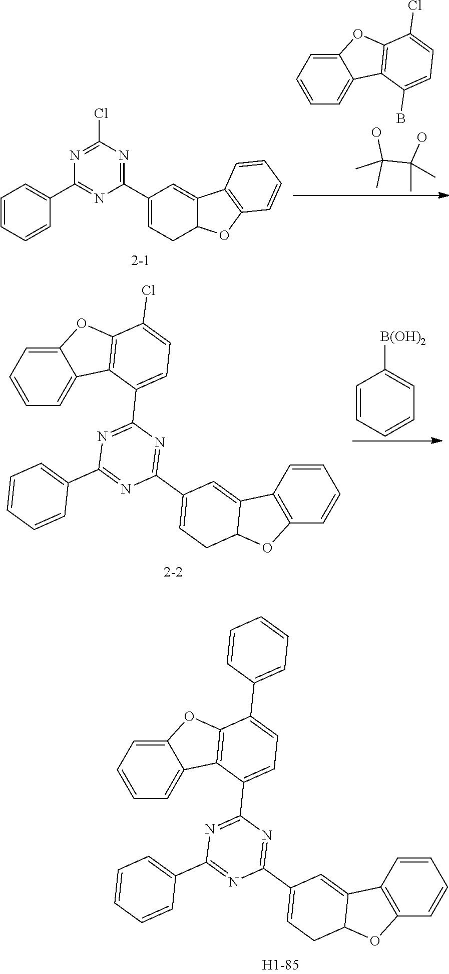

### 1. Synthesis of Compound 2-2

Compound 2-1 (7.65 g, 23.28 mmol), 2-(4-chlorodibenzo[b,d]furan-1-yl)-4,4,5,5-tetramethyl-1,3,2-dioxaborolane (9.99 g, 27.94 mmol), tetrakis(triphenylphosphine)palladium(0) (1.35 g, 1.16 mmol), and potassium carbonate (9.65 g, 69.84 mmol) were added to a flask, and 116 mL of toluene, 26 mL of ethanol, and 26 mL of distilled water were added thereto and dissolved, and these were then stirred under reflux at 130° C. for 3 hours. After completion of the reaction, the mixture was cooled to room temperature, and the formed solid was filtered and then separated using column chromatography to obtain Compound 2-2 (10 g, yield: 82%).

### 2. Synthesis of Compound H1-85

Compound 2-2 (10 g, 19.09 mmol), phenylboronic acid (3.49 g, 28.63 mmol), tris(dibenzylideneacetone)dipalladium(0) (1.75 g, 1.91 mmol), SPhos (1.57 g, 3.82 mmol), cesium carbonate (18.66 g, 57.26 mmol), and 95 mL of xylene were added to a flask and dissolved, and these were then stirred under reflux at 160° C. for 1 hour. After completion of the reaction, the mixture was cooled to room temperature, the organic layer was extracted, and the remaining moisture was removed with magnesium sulfate. Thereafter, the residue was separated using column chromatography to obtain Compound H1-85 (2.3 g, yield: 21.3%).

| | MW | M.P. |
|---|---|---|
| H1-85 | 565.63 | 259.1° C. |

### [Example 3] Preparation of Compound H1-97

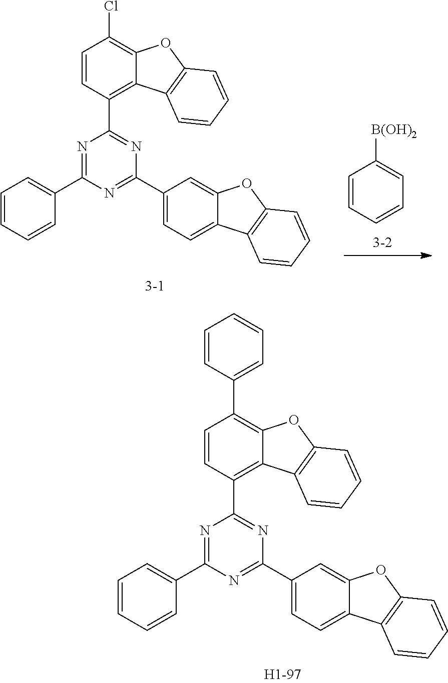 [SMILES: Clc1ccc(-c2nc(-c3ccccc3)nc(-c3ccc4c(c3)oc3ccccc34)n2)c2c1oc1ccccc12 ; OB(O)c1ccccc1 ; c1ccc(-c2nc(-c3ccc4c(c3)oc3ccccc34)nc(-c3ccc(-c4ccccc4)c4oc5ccccc5c34)n2)cc1]

Compound 3-1 (5 g, 9.543 mmol), Compound 3-2 (1.8 g, 14.31 mmol), Pd(OAc)₂ (0.21 g, 0.954 mmol), SPhos (0.78 g, 1.908 mmol), cesium carbonate (9.3 g, 28.63 mmol), 60 mL of o-xylene, 15 mL of 1,4-dioxane, and 15 mL of distilled water were added to a flask and dissolved, and these were then stirred under reflux at 160° C. for 3 hours. After completion of the reaction, the mixture was cooled to room temperature, methanol was added thereto, and the solid was filtered, dissolved in chlorobenzene, and then filtered through silica to obtain Compound H1-97 (2.4 g, yield: 44%).

| | MW | M.P. |
|---|---|---|
| H1-97 | 565.2 | 280.6° C. |

### [Example 4] Preparation of Compound H1-86

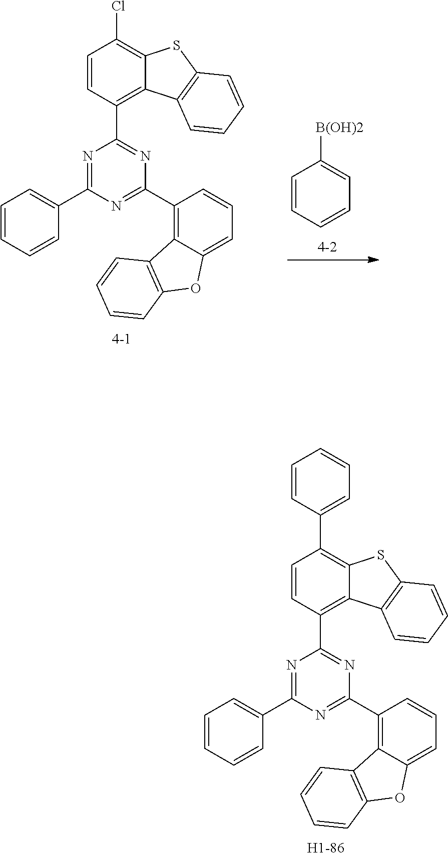 [SMILES: Clc1ccc(-c2nc(-c3ccccc3)nc(-c3cccc4oc5ccccc5c34)n2)c2c1sc1ccccc12 ; OB(O)c1ccccc1 ; c1ccc(-c2nc(-c3cccc4oc5ccccc5c34)nc(-c3ccc(-c4ccccc4)c4sc5ccccc5c34)n2)cc1]

Compound 4-1 (5.8 g, 10.74 mmol), Compound 4-2 (2 g, 16.11 mmol), Pd(OAc)₂ (0.24 g, 1.074 mmol), SPhos (0.88 g, 2.148 mmol), cesium carbonate (10.5 g, 32.22 mmol), 60 mL of o-xylene, 15 mL of 1,4-dioxane, and 15 mL of distilled water were added to a flask and dissolved, and these were then stirred at 160° C. for 3 hours. After completion of the reaction, the mixture was cooled to room temperature, methanol was added thereto, and the solid was filtered, dissolved in chlorobenzene, and then filtered through silica to obtain Compound H1-86 (1.9 g, yield: 30%).

| | MW | M.P. |
|---|---|---|
| H1-86 | 581.6 | 194.8° C. |

### [Example 5] Preparation of Compound H1-191

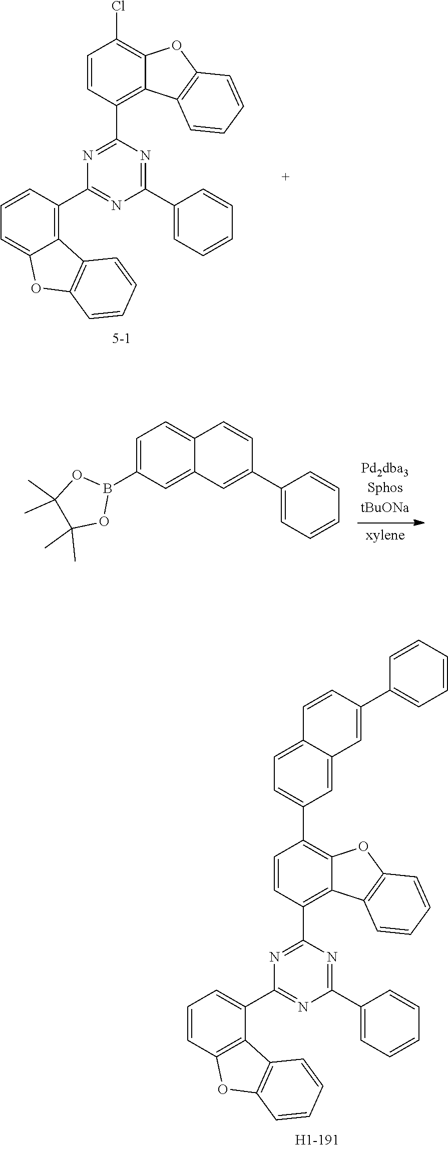 [SMILES: Clc1ccc(-c2nc(-c3ccccc3)nc(-c3cccc4oc5ccccc5c34)n2)c2c1oc1ccccc12 ; CC1(C)OB(c2ccc3ccc(-c4ccccc4)cc3c2)OC1(C)C ; c1ccc(-c2ccc3ccc(-c4ccc(-c5nc(-c6ccccc6)nc(-c6cccc7oc8ccccc8c67)n5)c5c4oc4ccccc45)cc3c2)cc1]

Compound 5-1 (3.8 g, 7.2 mmol), 4,4,5,5-tetramethyl-2-(7-phenylnaphthalene-2-yl)1,3,2-dioxaborolane (3.6 g, 11 mmol), Pd₂(dba)₃ (412 mg, 0.65 mmol), SPhos (0.53 g, 1.29 mmol), and NaOtBu (2.1 g, 22.5 mmol) were dissolved in 36 mL of o-xylene, and then stirred under reflux at 160° C. for 40 minutes. After completion of the reaction, the mixture was cooled to room temperature and filtered through Celite to obtain a solid. The solid was then separated using column chromatography to obtain Compound H1-191 (2.5 g, yield: 50.2%).

| | MW | M.P. |
|---|---|---|
| H1-191 | 691.77 | 280.3° C. |

### [Example 6] Preparation of Compound H1-201

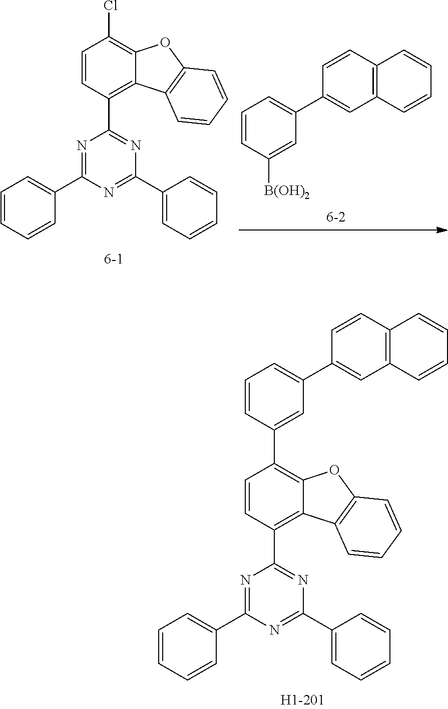

Compound 6-1 (5 g, 11.52 mmol), Compound 6-2 (2.9 g, 11.52 mmol), Pd(OAc)₂ (0.25 g, 1.152 mmol), SPhos (0.94 g, 2.305 mmol), cesium carbonate (11.2 g, 34.57 mmol), 80 mL of o-xylene, 20 mL of 1,4-dioxane, and 20 mL of distilled water were added to a flask and dissolved, and these were then stirred under reflux at 160° C. for 1 hour. After completion of the reaction, the mixture was cooled to room temperature, methanol was added thereto, and the solid was filtered, dissolved in o-xylene, and then filtered through silica to obtain Compound H1-201 (2.5 g, yield: 36%).

| | MW | M.P. |
|---|---|---|
| H1-201 | 601.6 | 264.6° C. |

### [Example 7] Preparation of Compound H1-202

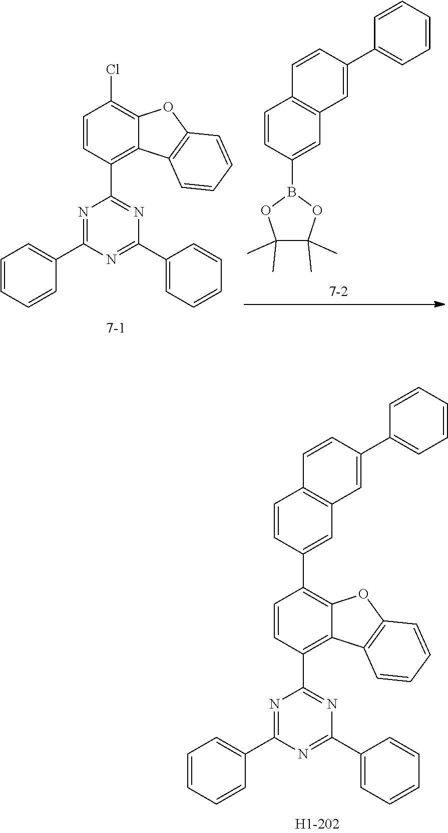

Compound 7-1 (9.4 g, 21.80 mmol), Compound 7-2 (7.2 g, 21.80 mmol), Pd(OAc)₂ (0.5 g, 2.180 mmol), SPhos (1.8 g, 4.360 mmol), cesium carbonate (21.3 g, 65.41 mmol), 140 mL of o-xylene, 35 mL of 1,4-dioxane, and 35 mL of distilled water were added to a flask and dissolved, and these were then stirred under reflux at 160° C. for 1 hour. After completion of the reaction, the mixture was cooled to room temperature, methanol was added thereto, and the solid was filtered, dissolved in o-xylene, and then filtered through silica to obtain Compound H1-202 (3.3 g, yield: 25%).

| | MW | M.P. |
|---|---|---|
| H1-202 | 601.6 | 265.5° C. |

### [Example 8] Preparation of Compound H1-43

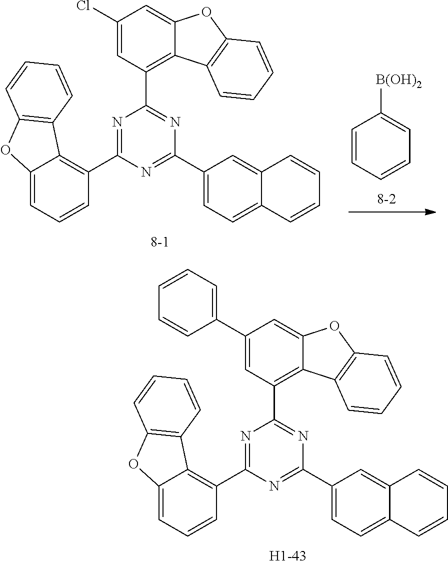

Compound 8-1 (8.3 g, 14.45 mmol), Compound 8-2 (4.6 g, 36.14 mmol), Pd(OAc)₂ (0.32 g, 1.445 mmol), SPhos (1.2 g, 2.891 mmol), cesium carbonate (14.1 g, 43.37 mmol), 80 mL of o-xylene, 20 mL of 1,4-dioxane, and 20 mL of distilled water were added to a flask and dissolved, and these were then stirred under reflux at 160° C. for 3 hours. After completion of the reaction, the mixture was cooled to room temperature, methanol was added thereto, and the solid was filtered, dissolved in chlorobenzene, and then filtered through silica to obtain Compound H1-43 (2.5 g, yield: 28%).

| | MW | M.P. |
|---|---|---|
| H1-43 | 615.6 | 211.4° C. |

### [Example 9] Preparation of Compound H1-194

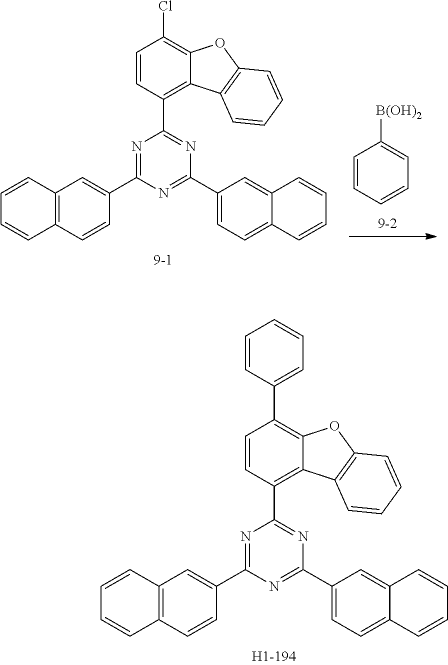

Compound 9-1 (5.2 g, 9.737 mmol), Compound 9-2 (3.7 g, 29.21 mmol), Pd(OAc)₂ (0.2 g, 0.973 mmol), SPhos (0.8 g, 1.947 mmol), cesium carbonate (9.5 g, 29.21 mmol), 60 mL of o-xylene, 15 mL of 1,4-dioxane, and 15 mL of distilled water were added to a flask and dissolved, and these were then stirred under reflux at 150° C. for 24 hours. After completion of the reaction, the mixture was cooled to room temperature, methanol was added thereto, and the solid was filtered, dissolved in o-xylene, and then filtered through silica to obtain Compound H1-194 (3.5 g, yield: 62%).

| | MW | M.P. |
|---|---|---|
| H1-194 | 575.6 | 271.1° C. |

### [Example 10] Preparation of Compound H1-225

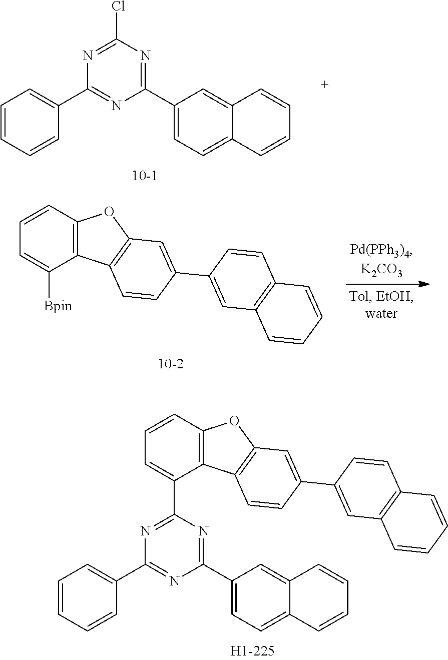

Compound 10-1 (5.1 g, 16.1 mmol), Compound 10-2 (7.5 g, 17.7 mmol), tetrakis(triphenylphosphine)palladium (1.0 g, 0.8 mmol), potassium carbonate (4.4 g, 32.1 mmol), 80 mL of toluene, 20 mL of ethanol, and 20 mL of distilled water were added to a flask and then stirred under reflux at 120° C. for 3 hours. After completion of the reaction, the mixture was cooled to room temperature, methanol was added thereto, and the solid was filtered. Thereafter, the resulting solid was purified using column chromatography to obtain Compound H1-225 (6.6 g, yield: 66%).

| | MW | M.P. |
|---|---|---|
| H1-225 | 575.7 | 229° C. |

### [Example 11] Preparation of Compound H1-73

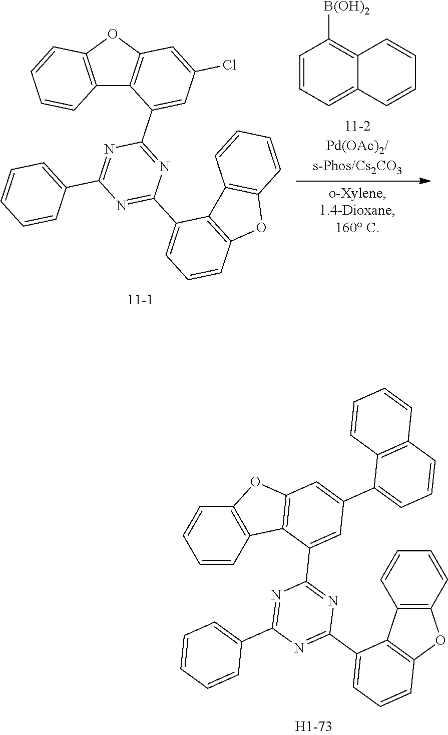 [SMILES: Clc1cc(-c2nc(-c3ccccc3)nc(-c3cccc4oc5ccccc5c34)n2)c2c(c1)oc1ccccc12 ; OB(O)c1cccc2ccccc12 ; c1ccc(-c2nc(-c3cccc4oc5ccccc5c34)nc(-c3cc(-c4cccc5ccccc45)cc4oc5ccccc5c34)n2)cc1]

Compound 11-1 (15 g, 28.63 mmol), Compound 11-2 (7.4 g, 42.94 mmol), palladium(II) acetate (0.64 g, 2.86 mmol), 2-dicyclohexylphosphino-2′,6′-dimethoxybiphenyl (2.4 g, 5.73 mmol), cesium carbonate (27.98 g, 85.88 mmol), 140 mL of o-xylene, and 36 mL of 1,4-dioxane were added to a flask, and 36 mL of distilled water was added thereto, and these were then stirred under reflux at 160° C. for 3 hours. After completion of the reaction, the mixture was cooled to room temperature, the organic layer was extracted with ethyl acetate, the extracted organic layer was dried with magnesium sulfate, and the solvent was removed using a rotary evaporator. Afterwards, the residue was purified using column chromatography to obtain Compound H1-73 (4.8 g, yield: 27%).

| MW | M.P. |
|---|---|
| H1-73 | 615.69 |

### [Example 12] Preparation of Compound H1-77

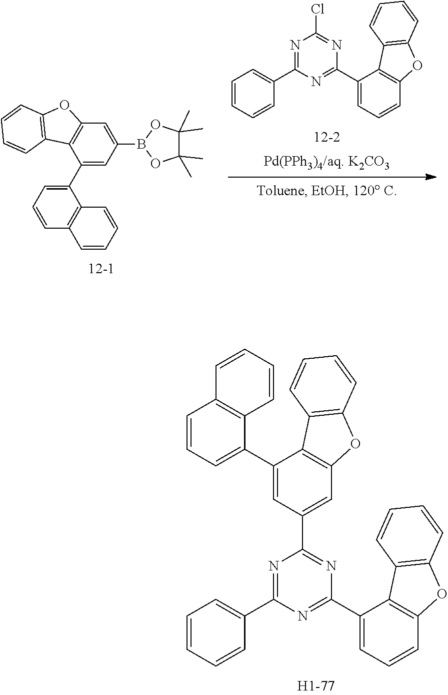 [SMILES: CC1(C)OB(c2cc(-c3cccc4ccccc34)c3c(c2)oc2ccccc23)OC1(C)C ; Clc1nc(-c2ccccc2)nc(-c2cccc3oc4ccccc4c23)n1 ; c1ccc(-c2nc(-c3cc(-c4cccc5ccccc45)c4c(c3)oc3ccccc34)nc(-c3cccc4oc5ccccc5c34)n2)cc1]

Compound 12-1 (10 g, 23.79 mmol), Compound 12-2 (8.5 g, 23.79 mmol), tetrakis(triphenylphosphine)palladium (0.8 g, 0.71 mmol), potassium carbonate (8.2 g, 59.48 mmol), 120 mL of toluene, 30 mL of ethanol, and 30 mL of distilled water were added to a flask and then stirred under reflux at 120° C. for 2 hours. After completion of the reaction, the mixture was cooled to room temperature, the organic layer was extracted with ethyl acetate, the extracted organic layer was dried with magnesium sulfate, and the solvent was removed using a rotary evaporator. Afterwards, the residue was purified using column chromatography to obtain Compound H1-77 (1.8 g, yield: 12%).

| | MW | M.P |
|---|---|---|
| H1-77 | 615.69 | 231° C. |

### [Example 13] Preparation of Compound H1-226

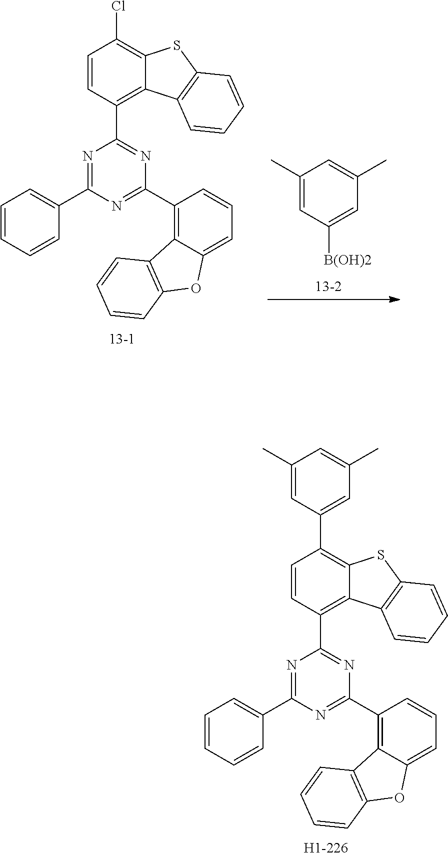

Compound 13-1 (5.4 g, 10.00 mmol), Compound 13-2 (2.2 g, 15.00 mmol), Pd(OAc)₂ (0.22 g, 1.000 mmol), SPhos (0.82 g, 2.000 mmol), cesium carbonate (9.7 g, 30.00 mmol), 60 mL of o-xylene, 15 mL of 1,4-dioxane, and 15 mL of distilled water were added to a flask and dissolved, and these were then stirred under reflux at 160° C. for 4 hours. After completion of the reaction, the reaction mixture was cooled to room temperature, the organic layer was extracted with ethyl acetate, the extracted organic layer was dried with magnesium sulfate, and the solvent was removed using a rotary evaporator. Afterwards, the residue was purified using column chromatography to obtain Compound H1-226 (4.5 g, yield: 73%).

| | MW | M.P. |
|---|---|---|
| H1-226 | 609.7 | 145.2° C. |

### [Example 14] Preparation of Compound H1-230

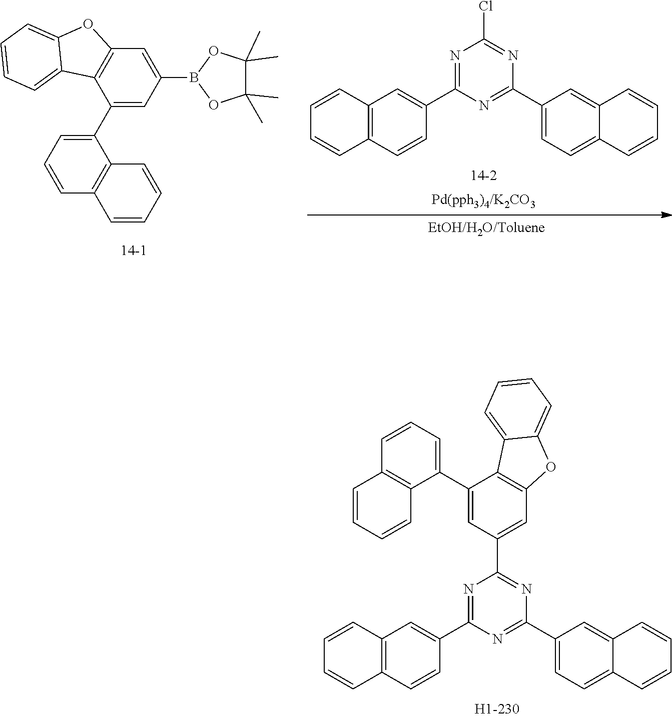

Compound 14-1 (15.7 g, 37.35 mmol), compound 14-2 (11 g, 29.88 mmol), Pd(pph₃)₄ (2.1 g, 1.867 mmol), K₂CO₃ (15.4 g, 112.0 mmol), 55 mL of EtOH, 55 mL of distilled water, and 220 mL of toluene were added to a flask and dissolved, and these were then stirred under reflux at 130° C. for 2 hours. After completion of the reaction, the mixture was cooled to room temperature, methanol was added thereto, and the solid was filtered, dissolved in chlorobenzene, and then filtered through silica to obtain Compound H1-230 (4 g, yield: 17%).

| | MW | M.P. |
|---|---|---|
| H1-230 | 625.7 | 248.5° C. |

### [Example 15] Preparation of Compound H1-233

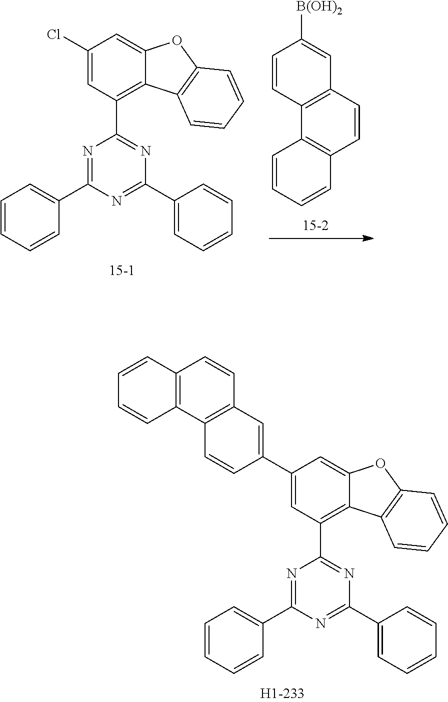

Compound 15-1 (5 g, 11.52 mmol), Compound 15-2 (4.5 g, 14.98 mmol), Pd(OAc)₂ (0.25 g, 1.152 mmol), SPhos (0.94 g, 2.305 mmol), cesium carbonate (11.2 g, 34.57 mmol), 80 mL of o-xylene, 20 mL of 1,4-dioxane, and 20 mL of distilled water were added to a flask and dissolved, and these were then stirred under reflux at 160° C. for 2 hours. After completion of the reaction, the mixture was cooled to room temperature, methanol was added thereto, and the solid was filtered, dissolved in o-xylene, and then filtered through silica to obtain Compound H1-233 (2.5 g, yield: 37%).

| | MW | M.P. |
|---|---|---|
| H1-233 | 575.6 | 287.9° C. |

### [Example 16] Preparation of Compound H1-237

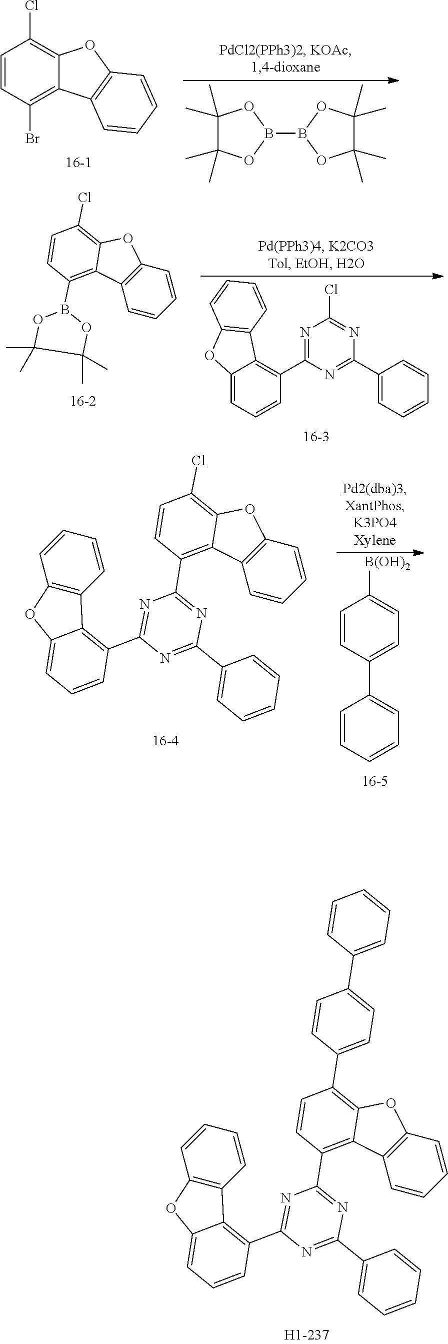

### 1. Synthesis of Compound 16-2

266 mL of 1,4-dioxane was added to Compound 16-1 (15 g, 53.28 mmol), bis(pinacolato)diboron (20.02 g, 79.92 mmol), PdCl₂(PPh₃)₂ (3.73 g, 5.3 mmol), and potassium acetate (13.07 g, 133.2 mmol) in a flask, and these were then stirred under reflux at 150° C. for 4 hours. After completion of the reaction, the mixture was cooled to room temperature and filtered through Celite. After distillation under reduced pressure, the residue was separated by column chromatography to obtain Compound 16-2 (8.3 g, yield: 47.4%).

### 2. Synthesis of Compound 16-4

90 mL of toluene, 23 mL of ethanol, and 23 mL of distilled water were added to Compound 16-2 (6.8 g, 20.6 mmol), Compound 16-3 (8.5 g, 23.7 mmol), Pd(PPh₃)₄ (1.2 g, 1.03 mmol), and potassium carbonate (7.1 g, 51.7 mmol) in a flask, and these were then stirred under reflux at 130° C. for 3.5 hours. After completion of the reaction, the mixture was cooled to room temperature, and the solid was filtered with methanol. The solid was then dissolved in chloroform and separated by column chromatography to obtain Compound 16-4 (9.1 g, yield: 83.9%).

### 3. Synthesis of Compound H1-237

40 mL of xylene was added to Compound 16-4 (3.8 g, 7.2 mmol), Compound 16-5 (1.7 g, 8.7 mmol), Pd₂(dba)₃ (0.3 g, 0.36 mmol), Xantphos (0.3 g, 0.7 mmol), and K₃PO₄ (4 g, 18.1 mmol) in a flask, and these were then stirred under reflux at 160° C. for 3.5 hours. After completion of the reaction, the mixture was cooled to room temperature, and the solid was filtered with methanol. The solid was then dissolved in chloroform and separated by column chromatography to obtain Compound H1-237 (3.0 g, yield: 65.2%).

| | MW | M.P. |
|---|---|---|
| H1-237 | 641.2 | 277.1° C. |

### [Example 17] Preparation of Compound H1-239

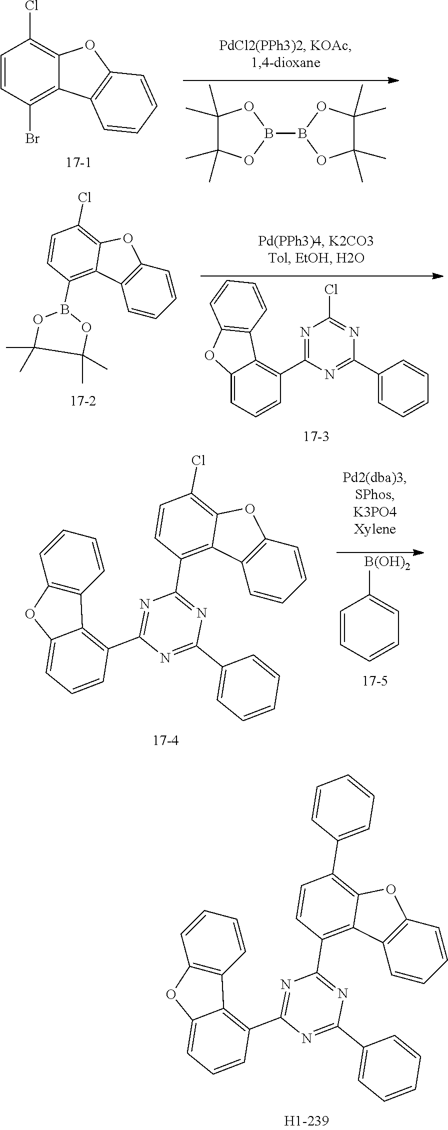

### 1. Synthesis of Compound 17-2

266 mL of 1,4-dioxane was added to Compound 17-1 (15 g, 53.28 mmol), bis(pinacolato)diboron (20.02 g, 79.92 mmol), PdCl₂(PPh₃)₂ (3.73 g, 5.3 mmol), and potassium acetate (13.07 g, 133.2 mmol) in a flask, and these were then stirred under reflux at 150° C. for 4 hours. After completion of the reaction, the mixture was cooled to room temperature and filtered through Celite. After distillation under reduced pressure, the residue was separated by column chromatography to obtain Compound 17-2 (8.3 g, yield: 47.4%).

### 2. Synthesis of Compound 17-4

90 mL of toluene, 23 mL of ethanol, and 23 mL of distilled water were added to Compound 17-2 (6.8 g, 20.6 mmol), Compound 17-3 (8.5 g, 23.7 mmol), Pd(PPh₃)₄ (1.2 g, 1.03 mmol), and potassium carbonate (7.1 g, 51.7 mmol) in a flask, and these were then stirred under reflux at 130° C. for 3.5 hours. After completion of the reaction, the mixture was cooled to room temperature, and the solid was filtered with methanol. The solid was then dissolved in chloroform and separated by column chromatography to obtain Compound 17-4 (9.1 g, yield: 83.9%).

### 3. Synthesis of Compound H1-239

40 mL of xylene was added to Compound 17-4 (4 g, 7.6 mmol), Compound 17-5 (1.02 g, 8.3 mmol), Pd₂(dba)₃ (0.3 g, 0.38 mmol), SPhos (0.3 g, 0.76 mmol), and K₃PO₄ (4.1 g, 19.0 mmol) in a flask, and these were then stirred under reflux at 160° C. for 3 hours. After completion of the reaction, the mixture was cooled to room temperature, distilled water was added thereto, and the organic layer was extracted with ethyl acetate. Afterwards, the solvent was removed by distillation under reduced pressure, and the solid was filtered, dried, and then dissolved in chloroform and separated by column chromatography to obtain Compound H1-239 (2.3 g, yield: 53.4%).

| | MW | M.P. |
|---|---|---|
| H1-239 | 565.1 | 258.3° C. |

### [Example 18] Preparation of Compound H1-198

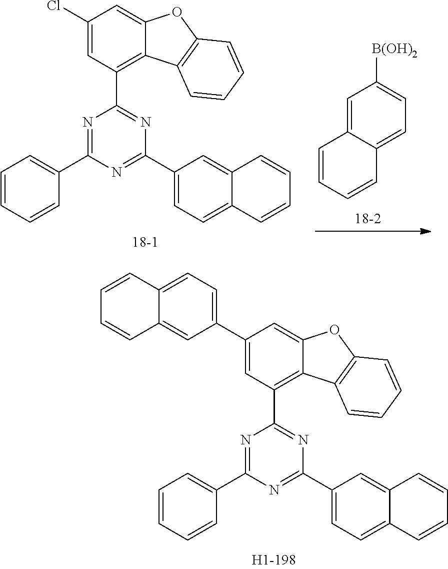

Compound 18-1 (7.5 g, 15.49 mmol), Compound 18-2 (3.5 g, 20.14 mmol), Pd(OAc)₂ (0.34 g, 1.549 mmol), SPhos (1.27 g, 3.099 mmol), cesium carbonate (15.1 g, 46.49 mmol), 80 mL of o-xylene, 20 mL of 1,4-dioxane, and 20 mL of distilled water were added to a flask and dissolved, and these were then stirred under reflux at 160° C. for 2 hours. After completion of the reaction, the mixture was cooled to room temperature, methanol was added thereto, and the solid was filtered, dissolved in o-xylene, and then filtered through silica to obtain Compound H1-198 (4.2 g, yield: 47%).

| | MW | M.P. |
|---|---|---|
| H1-198 | 575.6 | 259.7° C. |

### [Example 19] Preparation of Compound H1-69

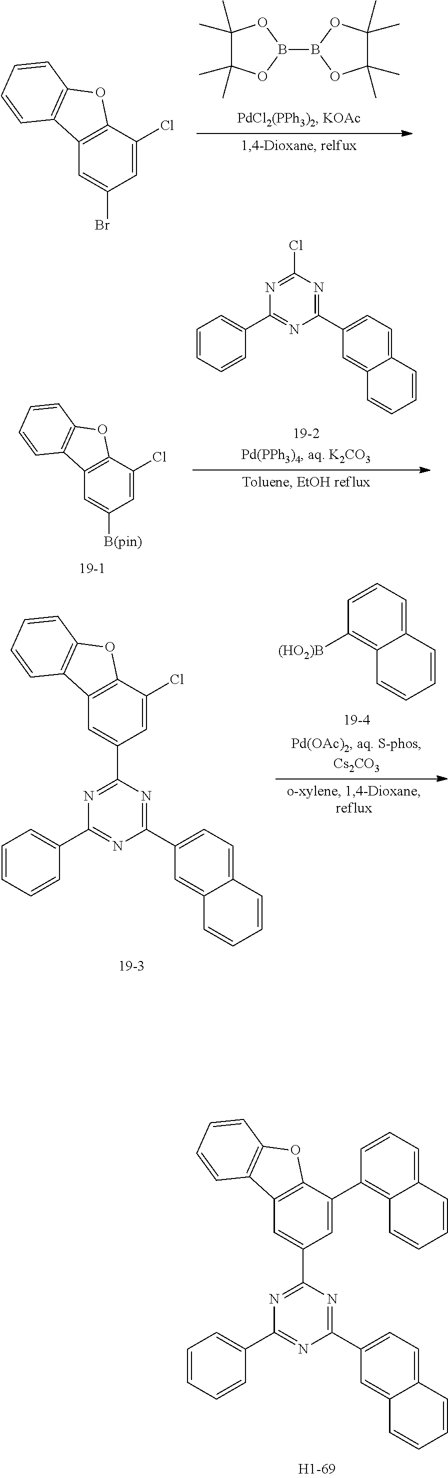

### 1. Synthesis of Compound 19-1

2-Bromo-4-chlorodibenzofuran (20.0 g, 71.05 mmol), bis(pinacolato)diboron (21.6 g, 85.26 mmol), PdCl₂(pph₃)₂ (5.0 g, 7.09 mmol), and KOAc (21.0 g, 213.2 mmol) were dissolved in a flask in 360 mL of 1,4-dioxane, and these were then stirred under reflux at 150° C. for 4 hours. After completion of the reaction, the mixture was cooled to room temperature, and the layers (EA/H₂O) were separated and filtered through Celite and then silica to obtain a solid. The solid was then filtered to obtain Compound 19-1 (19.8 g, yield: 85%).

### 2. Synthesis of Compound 19-3

120 mL of toluene, 30 mL of EtOH, and 30 mL of distilled water were added to Compound 19-1 (9.4 g, 28.6 mmol), Compound 19-2 (7.6 g, 23.84 mmol), Pd(pph₃)₄ (0.83 g, 0.72 mmol), and potassium carbonate (8.2 g, 59.6 mmol) in a flask, and these were then stirred under reflux at 130° C. for 4 hours. After completion of the reaction, the mixture was cooled to room temperature, and H₂O was added to the reaction product in which a solid has been produced. The mixture was then stirred for 30 minutes, filtered, and separated using column chromatography to obtain Compound 19-3 (10.4 g, yield: 72%).

### 3. Synthesis of Compound H1-69

Compound 19-3 (10.4 g, 21.5 mmol), Compound 19-4 (5.5 g, 32.25 mmol), Pd(OAc)₂ (0.97 g, 4.3 mmol), SPhos (3.5 g, 8.6 mmol), and cesium carbonate (28.0 g, 86.0 mmol) were dissolved in a flask in 215 mL of o-xylene, 27 mL of 1,4-dioxane, and 27 mL of distilled water, and these were then stirred under reflux at 160° C. for 4 hours. After completion of the reaction, the mixture was cooled to room temperature, and the layers (EA/H₂O) were separated and filtered through Celite and then silica to obtain a solid. The solid was then filtered to obtain Compound H1-69 (5.0 g, yield: 40.0%).

| | MW | M.P. |
|---|---|---|
| H1-69 | 575.7 | 278° C. |

### [Example 20] Preparation of Compound H1-58

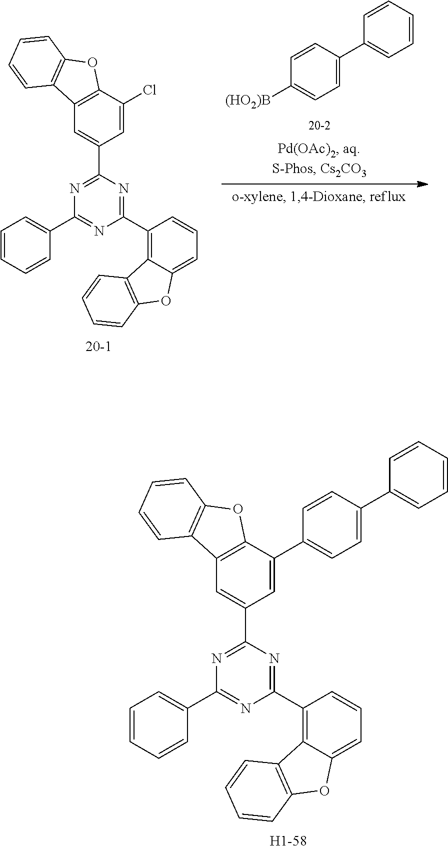

Compound 20-1 (5.4 g, 10.31 mmol), Compound 20-2 (3.0 g, 15.46 mmol), Pd(OAc)₂ (0.23 g, 1.03 mmol), SPhos (0.85 g, 2.06 mmol), and cesium carbonate (10.1 g, 30.93 mmol) in a flask were dissolved in 51 mL of o-xylene, 13 mL of 1,4-dioxane, and 13 mL of distilled water, and then stirred under reflux at 160° C. for 4 hours. After completion of the reaction, the mixture was cooled to room temperature, and the layers (EA/H₂O) were separated and filtered through Celite and then silica to obtain a solid. The solid was then filtered to obtain Compound H1-58 (2.0 g, yield: 30.0%)

| MW | M.P. |
|---|---|
| H1-58 | 641.7 |

### [Example 21] Preparation of Compound H1-240

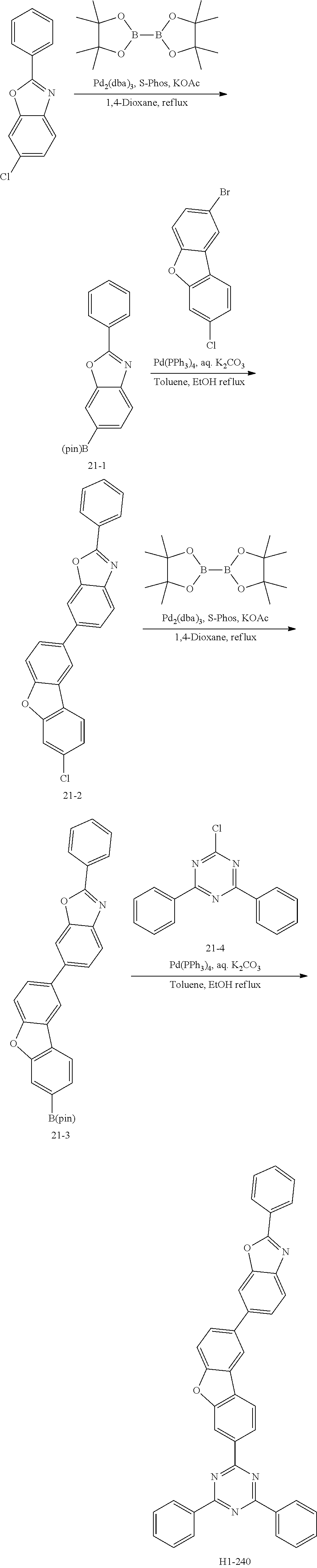

### 1. Synthesis of Compound 21-1

6-Chloro-2-phenylbenzoxazole (10.0 g, 43.54 mmol), bis(pinacolato)diboron (14.37 g, 56.61 mmol), Pd₂(dba)₃ (2.0 g, 2.18 mmol), SPhos (1.8 g, 4.35 mmol), and KOAc (12.8 g, 130.62 mmol) in a flask were dissolved in 220 mL of 1,4-dioxane, and these were then stirred under reflux at 150° C. for 3 hours. After completion of the reaction, the mixture was cooled to room temperature, and the layers (EA/H₂O) were separated and filtered through Celite and then silica to obtain a solid. The solid was then filtered to obtain Compound 21-1 (17.2 g, yield: 16.0%).

### 2. Synthesis of Compound 21-2

Compound 21-1 (17.2 g, 53.28 mmol), 2-bromo-7-chlorodibenzofuran (18 g, 63.93 mmol), Pd(pph₃)₄ (1.85 g, 1.6 mmol), and potassium carbonate (18.4 g, 133.2 mmol) in a flask were dissolved in 266 mL of toluene, 66 mL of EtOH, and 66 mL of distilled water, and these were then stirred under reflux at 130° C. for 4 hours. After completion of the reaction, the mixture was cooled to room temperature, H₂O was added to the reaction product in which a solid has been produced, and this was stirred for 30 minutes, filtered, and then separated using column chromatography to obtain Compound 21-2 (11.8 g, yield: 56.0%).

### 3. Synthesis of Compound 21-3

Compound 21-2 (11.8 g, 29.81 mmol), bis(pinacolato)diboron (9.8 g, 38.75 mmol), Pd₂(dba)₃ (1.36 g, 1.49 mmol), SPhos (1.2 g, 2.98 mmol), and KOAc (8.7 g, 89.43 mmol) in a flask were dissolved in 150 mL of 1,4-dioxane, and these were then stirred under reflux at 150° C. for 3 hours. After completion of the reaction, the mixture was cooled to room temperature, the layers (EA/H₂O) were separated and filtered through Celite and then silica to obtain a solid. The solid was then filtered to obtain Compound 21-3 (9.0 g, yield: 61.9%).

### 4. Synthesis of Compound H1-240

Compound 21-3 (9.0 g, 18.47 mmol), Compound 21-4 (5.9 g, 22.16 mmol), Pd(pph₃)₄ (0.6 g, 0.554 mmol), and potassium carbonate (6.4 g, 46.18 mmol) in a flask were dissolved in 100 mL of toluene, 25 mL of EtOH, and 25 mL of distilled water, and these were then stirred under reflux for 4 hours. After completion of the reaction, the mixture was cooled to room temperature, H₂O was added to the reaction product in which a solid has been produced, and this was stirred under reflux at 130° C. for 30 minutes. After completion of the reaction, the mixture was cooled to room temperature, filtered, and then separated by column chromatography to obtain Compound H1-240 (1.7 g, yield: 16.0%).

| | MW | M.P. |
|---|---|---|
| H1-240 | 592.64 | 340° C. |

### [Example 22] Preparation of Compound H1-262

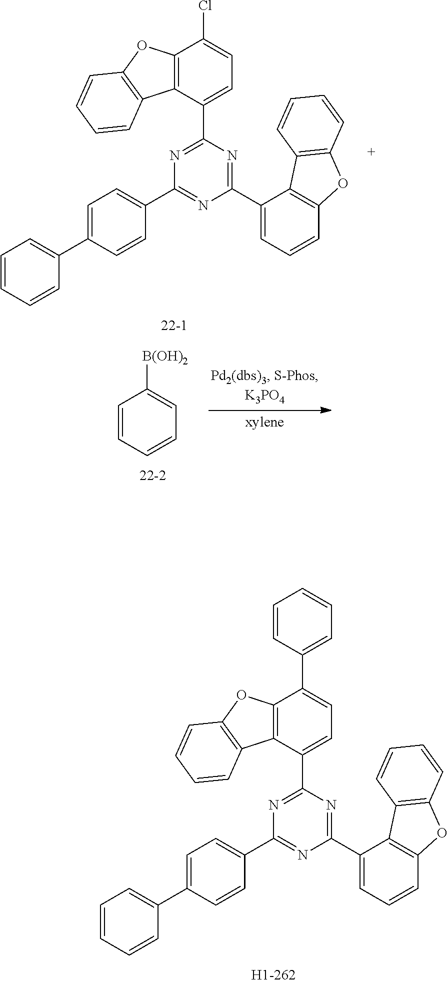 [SMILES: Clc1ccc(-c2nc(-c3ccc(-c4ccccc4)cc3)nc(-c3cccc4oc5ccccc5c34)n2)c2c1oc1ccccc12 ; OB(O)c1ccccc1 ; c1ccc(-c2ccc(-c3nc(-c4cccc5oc6ccccc6c45)nc(-c4ccc(-c5ccccc5)c5oc6ccccc6c45)n3)cc2)cc1]

Compound 22-1 (14 g, 23.3 mmol), Compound 22-2 (3.5 g, 27.96 mmol), Pd₂(dba)₃ (1 g, 1.165 mmol), SPhos (765 mg, 1.86 mmol), and 120 mL of xylene were added to a flask and dissolved, and these were then stirred under reflux at 160° C. for 1 hour. After completion of the reaction, the mixture was cooled to room temperature and then washed with distilled water, the organic layer was extracted with ethyl acetate, the extracted organic layer was dried with magnesium sulfate, and the solvent was removed. Afterwards, the residue was purified by column chromatography to obtain Compound H1-262 (3 g, yield: 20%).

| | MW | M.P. |
|---|---|---|
| H1-262 | 641.73 | 242.2° C. |

### [Example 23] Preparation of Compound H1-63

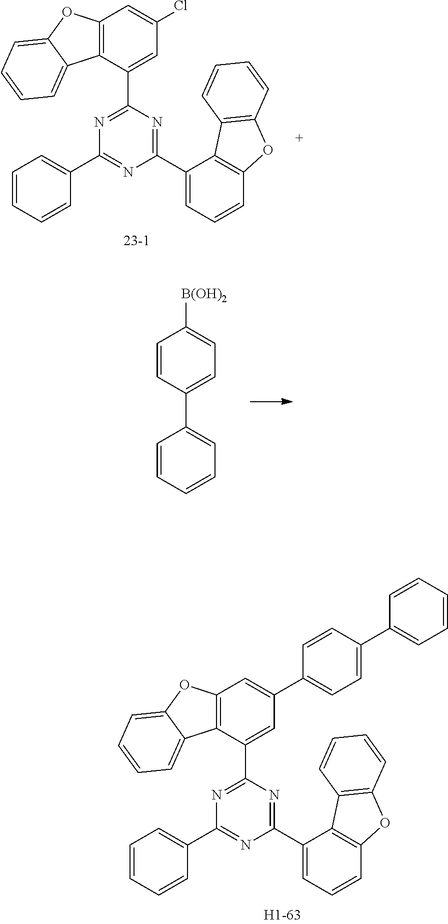 [SMILES: Clc1cc(-c2nc(-c3ccccc3)nc(-c3cccc4oc5ccccc5c34)n2)c2c(c1)oc1ccccc12 ; OB(O)c1ccc(-c2ccccc2)cc1 ; c1ccc(-c2ccc(-c3cc(-c4nc(-c5ccccc5)nc(-c5cccc6oc7ccccc7c56)n4)c4c(c3)oc3ccccc34)cc2)cc1]

61 mL of xylene, 16 mL of 1,4-dioxane, and 16 mL of distilled water were added to Compound 23-1 (6.43 g, 12.2 mmol), [1,1′-biphenyl]-4-yl boronic acid (3.65 g, 18.4 mmol), Pd₂(dba)₃ (1.13 g, 1.2 mmol), SPhos (1.01 g, 2.4 mmol), and cesium carbonate (12 g, 36.8 mmol) in a flask and dissolved, and these were then stirred under reflux at 170° C. for 2 hours. After completion of the reaction, the mixture was cooled to room temperature, and the resulting solid was filtered, washed with methanol, and dried. Afterwards, the solid was purified by column chromatography to obtain Compound H1-63 (2.23 g, yield: 28.2%).

| | MW | M.P. |
|---|---|---|
| H1-63 | 641.73 | 219.6° C. |

### [Example 24] Preparation of Compound H1-341

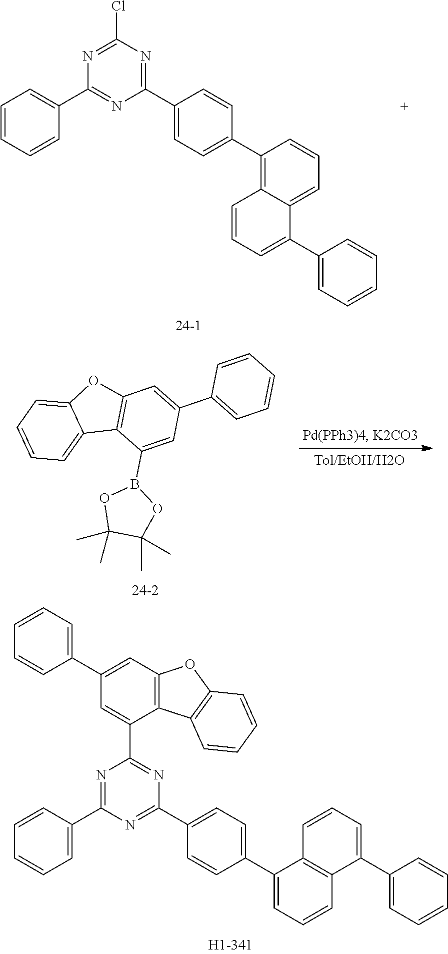 [SMILES: Clc1nc(-c2ccccc2)nc(-c2ccc(-c3cccc4c(-c5ccccc5)cccc34)cc2)n1 ; CC1(C)OB(c2cc(-c3ccccc3)cc3oc4ccccc4c23)OC1(C)C ; c1ccc(-c2cc(-c3nc(-c4ccccc4)nc(-c4ccc(-c5cccc6c(-c7ccccc7)cccc56)cc4)n3)c3c(c2)oc2ccccc23)cc1]

Compound 24-1 (8 g, 17 mmol), Compound 24-2 (6.9 g, 18.7 mmol), Pd(PPh₃)₄ (982 mg, 0.85 mmol), and K₂CO₃ (3.5 g, 22.5 mmol) in a flask were dissolved in 85 mL of toluene, 42.5 mL of EtOH, and 42.5 mL of distilled water, and these were then stirred under reflux at 130° C. for 1.5 hours. After completion of the reaction, the mixture was cooled to room temperature, H₂O was added to the reaction product in which a solid has been produced, and this was stirred for 30 minutes, filtered, and then recrystallized to obtain Compound H1-341 (3.7 g, yield: 32%).

| MW | M.P. |
|---|---|
| H1-341 | 677.81 |

Hereinafter, the preparation method of an organic electroluminescent device comprising the plurality of host materials according to the present disclosure and the device properties thereof will be explained.

### [Device Examples 1 to 13] Producing OLEDs Co-Deposited with the First Host Compound and Second Host Compound According to the Present Disclosure

OLEDs according to the present disclosure were produced. First, a transparent electrode indium tin oxide (ITO) thin film (10 Ω/sq) on a glass substrate for an OLED (GEOMATEC CO., LTD., Japan) was subjected to ultrasonic washing with acetone and isopropyl alcohol, sequentially, and thereafter was stored in isopropyl alcohol and then used. Thereafter, the ITO substrate was mounted on a substrate holder of a vacuum vapor deposition apparatus. Compound HI-1 was then introduced into a cell of the vacuum vapor deposition apparatus, and Compound HT-1 was introduced into another cell. The two materials were evaporated at different rates, and Compound HI-1 was deposited in a doping amount of 3 wt % based on the total amount of Compounds HI-1 and HT-1 to form a first hole injection layer having a thickness of 10 nm. Next, Compound HT-1 was deposited on the first hole injection layer to form a first hole transport layer having a thickness of 80 nm. Compound HT-2 was then introduced into another cell of the vacuum vapor deposition apparatus and was evaporated by applying an electric current to the cell, thereby forming a second hole transport layer having a thickness of 60 nm on the first hole transport layer.

After forming the hole injection layer and the hole transport layers, a light-emitting layer was formed thereon as follows: each of the first host compound and the second host compound described in the following Table 1 were introduced into two cells of the vacuum vapor deposition apparatus as hosts, respectively, and Compound D-39 was introduced into another cell as a dopant. The two host materials were evaporated at a rate of 1:1, and the dopant material was evaporated at a different rate, simultaneously, and the dopant was deposited in a doping amount of 3 wt % based on the total amount of the hosts and dopant to form a light-emitting layer having a thickness of 40 nm on the second hole transport layer. Next, Compounds ET-1 and EI-1 as electron transport materials were deposited at a weight ratio of 50:50 to form an electron transport layer having a thickness of 35 nm on the light-emitting layer. After depositing Compound EI-1 as an electron injection layer having a thickness of 2 nm on the electron transport layer, an Al cathode having a thickness of 80 nm was deposited on the electron injection layer by another vacuum vapor deposition apparatus. Thus, OLEDs were produced. All of the materials used for producing the OLEDs were purified by vacuum sublimation at 10⁻⁶ Torr.

### [Comparative Examples 1 to 3] Producing OLEDs Comprising the Conventional Compound as a Host

OLEDs were manufactured in the same manner as in Device Example 1, except that the compounds of the following Table 1 were used as the first host material of the light-emitting layer, respectively.

The driving voltage, the current efficiency, the luminous color at a luminance of 1,000 nit, and the time taken for luminance to decrease from 100% to 95% at a luminance of 10,000 nit (lifespan: T₉₅) of the OLEDs produced in Device Examples 1 to 8 and Comparative Examples 1 to 3 were measured, and the results thereof are shown in Table 1 below.

TABLE 1

| | | | Driving | Current | | Lifespan |
|---|---|---|---|---|---|---|
| | First | Second | Voltage | Efficiency | Luminous | T₉₅ |
| | Host | Host | (V) | (cd/A) | Color | (hr) |
| Device | H1-73 | H2-144 | 2.9 | 34.3 | Red | 246 |
| Example 1 | | | | | | |
| Device | H1-77 | H2-144 | 2.9 | 32.5 | Red | 174 |
| Example 2 | | | | | | |
| Device | H1-73 | H3-54 | 2.9 | 35.7 | Red | 165 |
| Example 3 | | | | | | |
| Device | H1-77 | H3-54 | 2.9 | 34.5 | Red | 232 |
| Example 4 | | | | | | |
| Device | H1-44 | H3-54 | 2.8 | 32.8 | Red | 210 |
| Example 5 | | | | | | |
| Device | H1-63 | H3-54 | 2.9 | 34.8 | Red | 304 |
| Example 6 | | | | | | |
| Device | H1-73 | H4-105 | 2.8 | 34.8 | Red | 107 |
| Example 7 | | | | | | |
| Device | H1-77 | H4-105 | 2.8 | 34.6 | Red | 239 |
| Example 8 | | | | | | |
| Device | H1-262 | H2-144 | 2.9 | 34.0 | Red | 264 |
| Example 9 | | | | | | |
| Device | H1-115 | H2-144 | 2.8 | 32.9 | Red | 356 |
| Example 10 | | | | | | |
| Device | H1-126 | H2-144 | 2.8 | 32.9 | Red | 272 |
| Example 11 | | | | | | |
| Device | H1-330 | H2-144 | 3.0 | 34.2 | Red | 266 |
| Example 12 | | | | | | |
| Device | H1-333 | H2-144 | 2.9 | 33.1 | Red | 328 |
| Example 13 | | | | | | |
| Comparative | C-1 | H2-144 | 3.1 | 33.0 | Red | 48 |
| Example 1 | | | | | | |
| Comparative | C-1 | H3-54 | 3.0 | 31.1 | Red | 34 |
| Example 2 | | | | | | |
| Comparative | C-1 | H4-105 | 2.9 | 30.3 | Red | 20 |
| Example 3 | | | | | | |

From Table 1 above, it can be confirmed that an organic electroluminescent device including a specific combination of compounds according to the present disclosure as a host material has, for example, higher luminescence efficiency and/or longer lifespan compared to an organic electroluminescent device using a conventional host material.

### [Device Examples 14 to 31] Producing OLEDs Comprising a Plurality of Host Materials According to the Present Disclosure

OLEDs were manufactured in the same manner as Device Example 1, except that Compound HT-3 was used for a first hole injection layer, Compound HT-4 was used to form a second hole transport layer with a thickness of 55 nm, and Compound HT-5 was deposited thereon to form a third hole transport layer with a thickness of 5 nm, the thickness of the electron transport layer was reduced to 30 nm, the first host, the second host, and the third host described in the following Table 2 were deposited at a ratio of 2.5:5:2.5 as host materials for the light-emitting layer, Compound Ir-D was used as a dopant, and Compound BF-1 was deposited between the light-emitting layer and the electron transport layer to form an electron buffer layer with a thickness of 5 nm, and Compound ET-2 was used in the electron transport layer.

The driving voltage, the current efficiency, the luminous color at a luminance of 1,000 nit, and the time taken for luminance to decrease from 100% to 95% at a luminance of 15,000 nit (lifetime: T₉₅) of the OLEDs produced in Device Examples 14 to 31 were measured, and the results thereof are shown in Table 2 below.

TABLE 2

| | | | | Driving | Current | | Lifetime |
|---|---|---|---|---|---|---|---|
| | First | Second | Third | Voltage | Efficiency | Luminous | (T₉₅, |
| | Host | Host | Host | (V) | (cd/A) | Color | hr) |
| Device | H1-44 | H2-173 | H5-28 | 2.9 | 31.0 | Red | 160 |
| Example 14 | | | | | | | |
| Device | H1-44 | H2-144 | H5-28 | 2.9 | 31.1 | Red | 120 |
| Example 15 | | | | | | | |
| Device | H1-44 | H3-193 | H5-28 | 2.9 | 30.3 | Red | 179 |
| Example 16 | | | | | | | |
| Device | H1-63 | H2-173 | H5-28 | 2.9 | 31.4 | Red | 176 |
| Example 17 | | | | | | | |
| Device | H1-63 | H2-144 | H5-28 | 3.0 | 31.1 | Red | 123 |
| Example 18 | | | | | | | |
| Device | H1-63 | H3-193 | H5-28 | 2.9 | 30.2 | Red | 165 |
| Example 19 | | | | | | | |
| Device | H1-73 | H2-173 | H5-28 | 2.9 | 31.5 | Red | 197 |
| Example 20 | | | | | | | |
| Device | H1-73 | H2-144 | H5-28 | 3.0 | 30.9 | Red | 136 |
| Example 21 | | | | | | | |
| Device | H1-73 | H3-193 | H5-28 | 2.9 | 29.9 | Red | 176 |
| Example 22 | | | | | | | |
| Device | H1-44 | H2-173 | H5-29 | 2.9 | 31.6 | Red | 243 |
| Example 23 | | | | | | | |
| Device | H1-44 | H2-144 | H5-29 | 3.0 | 31.2 | Red | 170 |
| Example 24 | | | | | | | |
| Device | H1-44 | H3-193 | H5-29 | 2.9 | 30.6 | Red | 294 |
| Example 25 | | | | | | | |
| Device | H1-63 | H2-173 | H5-29 | 3.0 | 31.4 | Red | 291 |
| Example 26 | | | | | | | |
| Device | H1-63 | H2-144 | H5-29 | 3.0 | 31.3 | Red | 192 |
| Example 27 | | | | | | | |
| Device | H1-63 | H3-193 | H5-29 | 3.0 | 30.9 | Red | 322 |
| Example 28 | | | | | | | |
| Device | H1-73 | H2-173 | H5-29 | 3.0 | 31.1 | Red | 280 |
| Example 29 | | | | | | | |
| Device | H1-73 | H2-144 | H5-29 | 3.0 | 31.1 | Red | 200 |
| Example 30 | | | | | | | |
| Device | H1-73 | H3-193 | H5-29 | 3.0 | 30.0 | Red | 298 |
| Example 31 | | | | | | | |

The compounds used in the Device Examples and the Comparative Examples are specifically shown in Table 3 below.

TABLE 3

| | | |
|---|---|---|
| Hole Injection Layer/Hole Transport Layer | 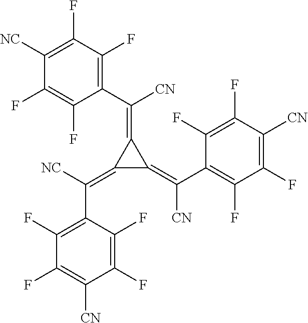 | HI-1 |
| | 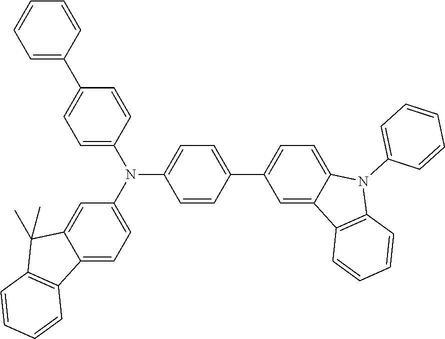 | HT-1 |
| | 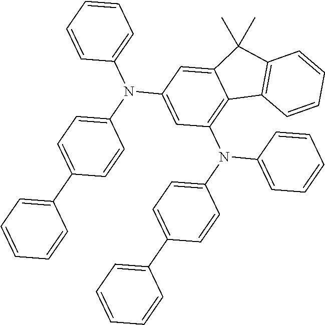 | HT-2 |
| | 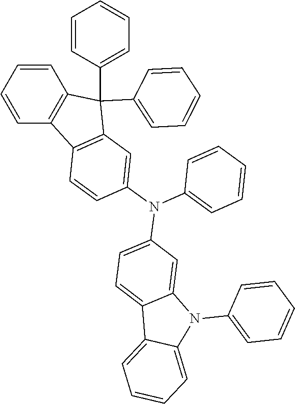 | HT-3 |
| | 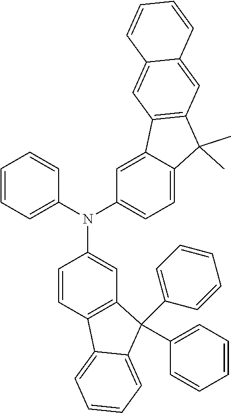 | HT-4 |
| | 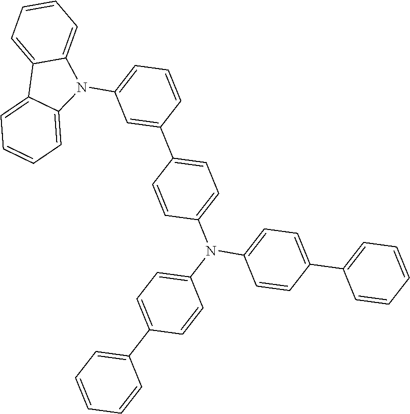 | HT-5 |
| Light- Emitting Layer | 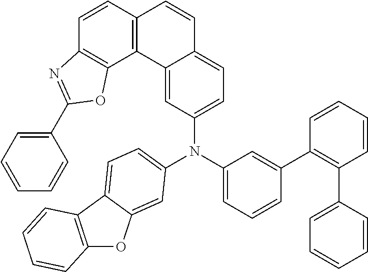 | H2-144 |
| | 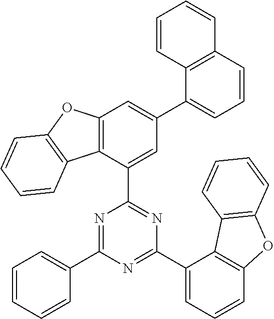 | H1-73 |
| | 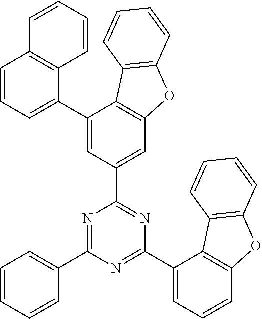 | H1-77 |
| | 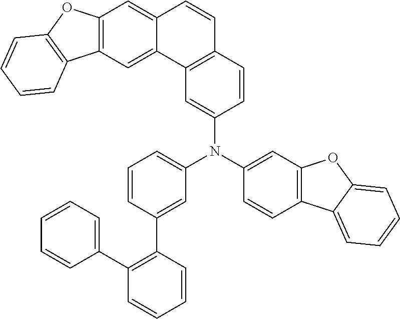 | H3-54 |
| | 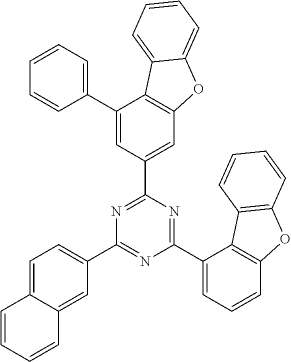 | H1-44 |
| | 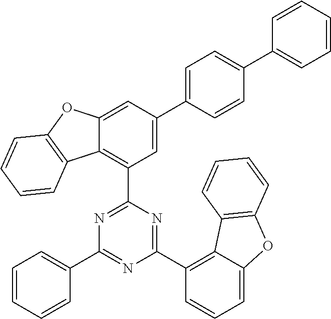 | H1-63 |
| | 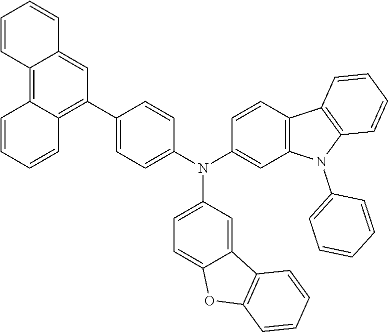 | H4-105 |
| | 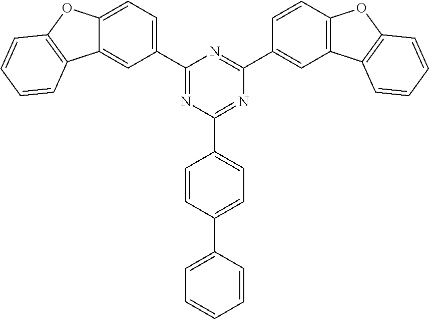 | C-1 |
| | 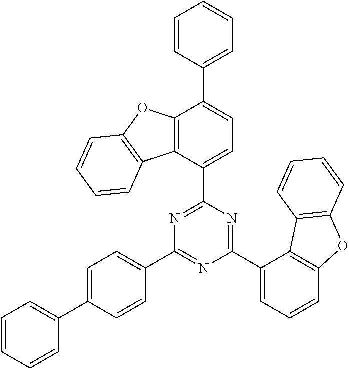 | H1-262 |
| | 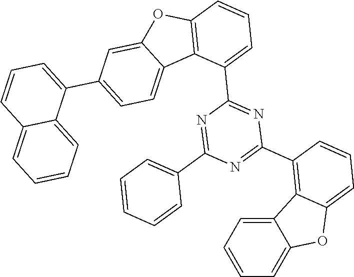 | H1-115 |
| | 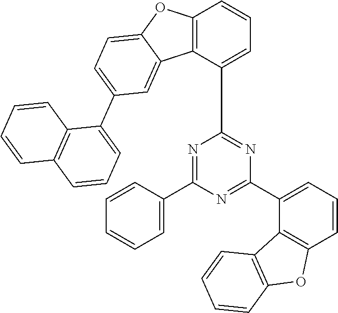 | H1-126 |
| | 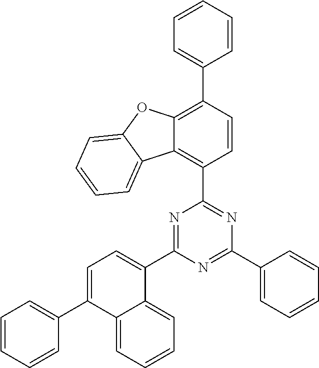 | H1-330 |
| | 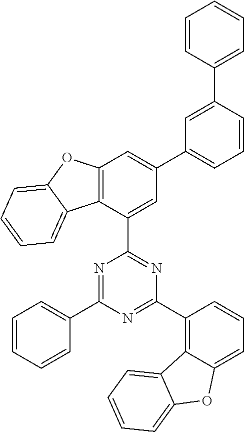 | H1-333 |
| | 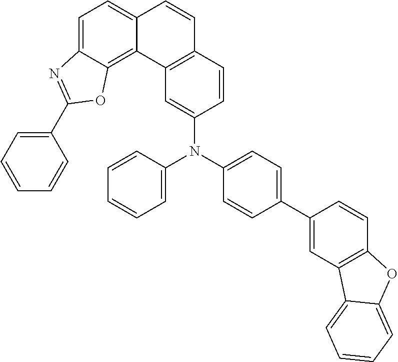 | H2-173 |
| | 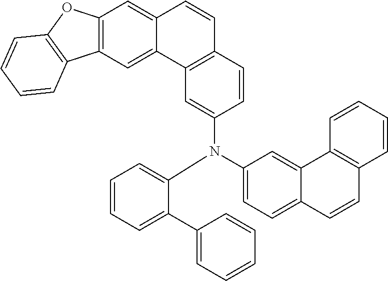 | H3-193 |
| | 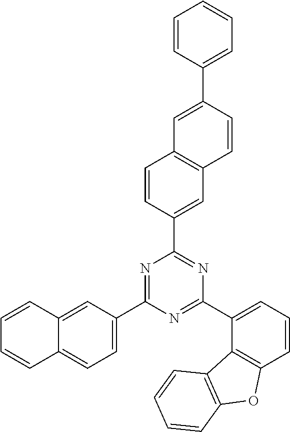 | H5-28 |
| | 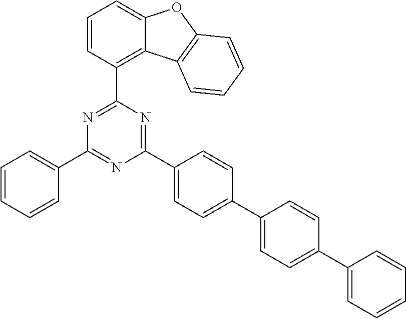 | H5-29 |
| | 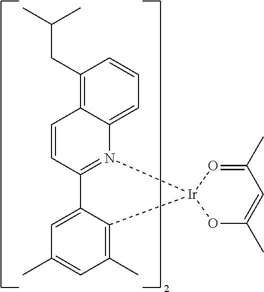 | D-39 |
| | 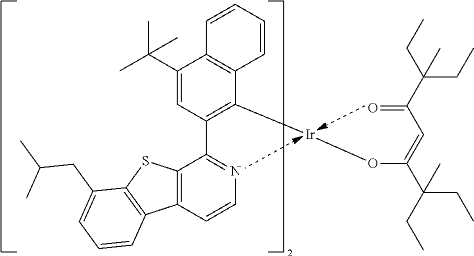 | Ir-D |
| Electron Transport Layer/ Electron Injection Layer | 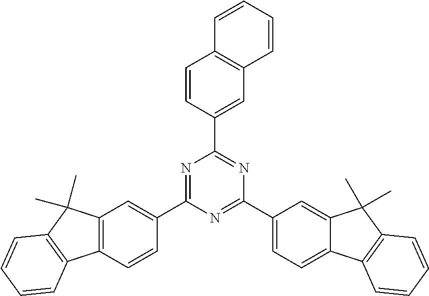 | ET-1 |
| | 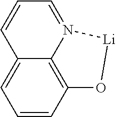 | EI-1 |
| | 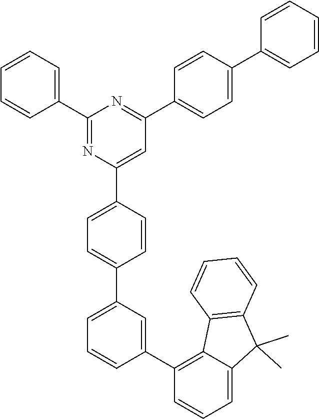 | BF-1 |
| | 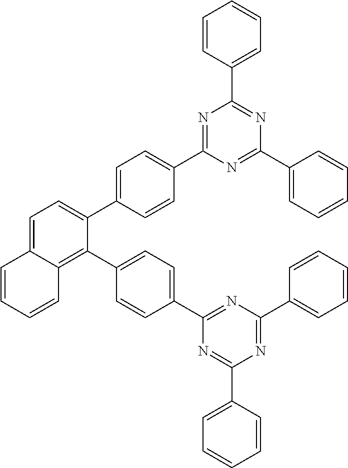 | ET-2 |

1. A plurality of host materials comprising a first host material and a second host compound, wherein the first host material comprises a compound represented by the following Formula 1, and the second host material comprises a compound represented by the following Formula 2: 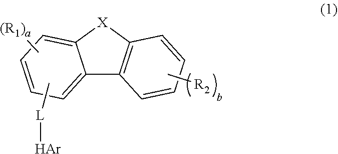
in Formula 1,
X represents O, S, or Se;
HAr represents a substituted or unsubstituted (3- to 30-membered)heteroaryl containing one or more nitrogen atoms;
L represents a single bond, a substituted or unsubstituted (C₆-C₃₀)arylene, or a substituted or unsubstituted (3- to 30-membered)heteroarylene;
R₁ and R₂ each independently represent hydrogen, deuterium, a halogen, a cyano, a substituted or unsubstituted (C₁-C₃₀)alkyl, a substituted or unsubstituted (C₆-C₃₀)aryl, a substituted or unsubstituted (3- to 30-membered)heteroaryl, a substituted or unsubstituted (C₃-C₃₀)cycloalkyl, a substituted or unsubstituted (C₁-C₃₀)alkoxy, a substituted or unsubstituted tri(C₁-C₃₀)alkylsilyl, a substituted or unsubstituted di(C₁-C₃₀)alkyl(C₆-C₃₀)arylsilyl, a substituted or unsubstituted (C₁-C₃₀)alkyldi(C₆-C₃₀)arylsilyl, or a substituted or unsubstituted tri(C₆-C₃₀)arylsilyl; or may be linked to the adjacent substituents to form a ring(s);
provided that at least one of R₁ and R₂ is a substituted or unsubstituted (C₆-C₃₀)aryl, or a substituted or unsubstituted (3- to 30-membered)heteroaryl; and
a represents an integer of 1 to 3, b represents an integer of 1 to 4, and when a and b are an integer of 2 or more, each of R₁ and each of R₂ may be the same as or different from each other; 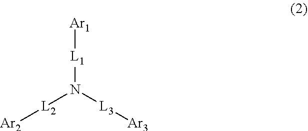
in Formula 2,
L₁ to L₃ each independently represent a single bond, a substituted or unsubstituted (C₆-C₃₀)arylene, or a substituted or unsubstituted (3- to 30-membered)heteroarylene;
Ar₁ represents a substituted or unsubstituted (C₆-C₃₀)aryl, or a substituted or unsubstituted (3- to 30-membered)heteroaryl; and
Ar₂ and Ar₃ each independently represent hydrogen, deuterium, a halogen, a cyano, a substituted or unsubstituted (C₁-C₃₀)alkyl, a substituted or unsubstituted (C₆-C₃₀)aryl, a substituted or unsubstituted (3- to 30-membered)heteroaryl, a substituted or unsubstituted (C₃-C₃₀)cycloalkyl, a substituted or unsubstituted (C₁-C₃₀)alkoxy, a substituted or unsubstituted tri(C₁-C₃₀)alkylsilyl, a substituted or unsubstituted di(C₁-C₃₀)alkyl(C₆-C₃₀)arylsilyl, a substituted or unsubstituted (C₁-C₃₀)alkyldi(C₆-C₃₀)arylsilyl, a substituted or unsubstituted tri(C₆-C₃₀)arylsilyl, a substituted or unsubstituted mono- or di(C₁-C₃₀)alkylamino, a substituted or unsubstituted mono- or di(C₂-C₃₀)alkenylamino, a substituted or unsubstituted (C₁-C₃₀)alkyl(C₂-C₃₀)alkenylamino, a substituted or unsubstituted mono- or di(C₆-C₃₀)arylamino, a substituted or unsubstituted (C₁-C₃₀)alkyl(C₆-C₃₀)arylamino, a substituted or unsubstituted mono- or di(3- to 30-membered)heteroarylamino, a substituted or unsubstituted (C₁-C₃₀)alkyl(3- to 30-membered)heteroarylamino, a substituted or unsubstituted (C₂-C₃₀)alkenyl(C₆-C₃₀)arylamino, a substituted or unsubstituted (C₂-C₃₀)alkenyl(3- to 30-membered)heteroarylamino, a substituted or unsubstituted (C₆-C₃₀)aryl(3- to 30-membered)heteroarylamino, or a substituted or unsubstituted fused ring of (C₃-C₃₀) aliphatic ring and (C₆-C₃₀) aromatic ring; or may be linked to the adjacent substituents to form a ring(s).

2. The plurality of host materials according to claim 1, wherein the substituted alkyl, the substituted aryl(ene), the substituted heteroaryl(ene), the substituted cycloalkyl, the substituted alkoxy, the substituted trialkylsilyl, the substituted dialkylarylsilyl, the substituted alkyldiarylsilyl, the substituted triarylsilyl, the substituted mono- or di-alkylamino, the substituted mono- or di-alkenylamino, the substituted alkylalkenylamino, the substituted mono- or di-arylamino, the substituted alkylarylamino, the substituted mono- or di-heteroarylamino, the substituted alkylheteroarylamino, the substituted alkenylarylamino, the substituted alkenylheteroarylamino, the substituted arylheteroarylamino, and the substituted fused ring of aliphatic ring and aromatic ring each independently are substituted with at least one selected from the group consisting of deuterium, a halogen, a cyano, carboxyl, nitro, hydroxyl, (C₁-C₃₀)alkyl, halo(C₁-C₃₀)alkyl, (C₂-C₃₀)alkenyl, (C₂-C₃₀)alkynyl, (C₁-C₃₀)alkoxy, (C₁-C₃₀)alkylthio, (C₃-C₃₀)cycloalkyl, (C₃-C₃₀)cycloalkenyl, (3- to 7-membered)heterocycloalkyl, (C₆-C₃₀)aryloxy, (C₆-C₃₀)arylthio, (5- to 30-membered)heteroaryl unsubstituted or substituted with (C₆-C₃₀)aryl, (C₆-C₃₀)aryl unsubstituted or substituted with (5- to 30-membered)heteroaryl, tri(C₁-C₃₀)alkylsilyl, tri(C₆-C₃₀)arylsilyl, di(C₁-C₃₀)alkyl(C₆-C₃₀)arylsilyl, (C₁-C₃₀)alkyldi(C₆-C₃₀)arylsilyl, a fused ring of (C₃-C₃₀) aliphatic ring and (C₆-C₃₀) aromatic ring, amino, mono- or di(C₁-C₃₀)alkylamino, mono- or di(C₆-C₃₀)arylamino unsubstituted or substituted with (C₁-C₃₀)alkyl, (C₁-C₃₀)alkyl(C₆-C₃₀)arylamino, mono- or di(3- to 30-membered)heteroarylamino, (C₁-C₃₀)alkyl(3- to 30-membered)heteroarylamino, (C₆-C₃₀)aryl(3- to 30-membered)heteroarylamino, (C₁-C₃₀)alkylcarbonyl, (C₁-C₃₀)alkoxycarbonyl, (C₆-C₃₀)arylcarbonyl, (C₆-C₃₀)arylphosphinyl, di(C₆-C₃₀)arylboronyl, di(C₁-C₃₀)alkylboronyl, (C₁-C₃₀)alkyl(C₆-C₃₀)arylboronyl, (C₆-C₃₀)ar(C₁-C₃₀)alkyl, and (C₁-C₃₀)alkyl(C₆-C₃₀)aryl.

3. The plurality of host materials according to claim 1, wherein Formula 1 is represented by any one of the following Formulas 1-1 to 1-4: 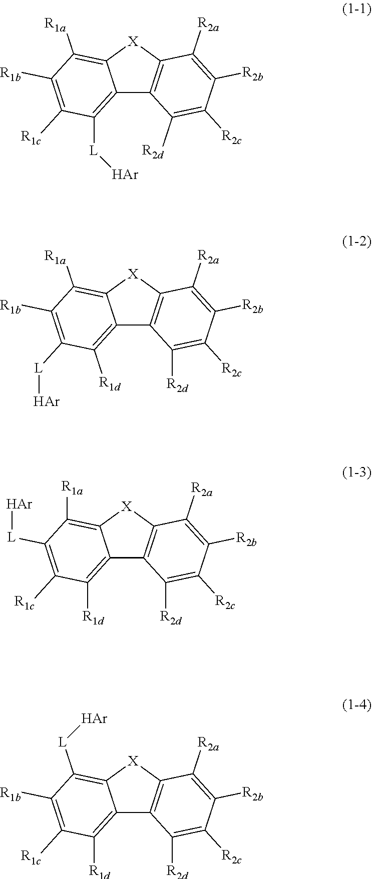
in Formulas 1-1 to 1-4,
R₁ₐ to R_{1d} and R₂ₐ to R_{2d} each independently represent hydrogen, deuterium, a halogen, a cyano, a substituted or unsubstituted (C₁-C₃₀)alkyl, a substituted or unsubstituted (C₆-C₃₀)aryl, a substituted or unsubstituted (3- to 30-membered)heteroaryl, a substituted or unsubstituted (C₃-C₃₀)cycloalkyl, a substituted or unsubstituted (C₁-C₃₀)alkoxy, a substituted or unsubstituted tri(C₁-C₃₀)alkylsilyl, a substituted or unsubstituted di(C₁-C₃₀)alkyl(C₆-C₃₀)arylsilyl, a substituted or unsubstituted (C₁-C₃₀)alkyldi(C₆-C₃₀)arylsilyl, or a substituted or unsubstituted tri(C₆-C₃₀)arylsilyl; or may be linked to the adjacent substituents to form a ring(s);
provided that in each of Formulas 1-1 to 1-4, at least one of R₁ₐ to R_{1d} and R₂ₐ to R_{2d} is a substituted or unsubstituted (C₆-C₃₀)aryl or a substituted or unsubstituted (3- to 30-membered)heteroaryl; and
X, L, and HAr are as defined in claim 1.

4. The plurality of host materials according to claim 1, wherein the compound represented by Formula 2 is represented by any one of the following Formulas 2-1 to 2-3: 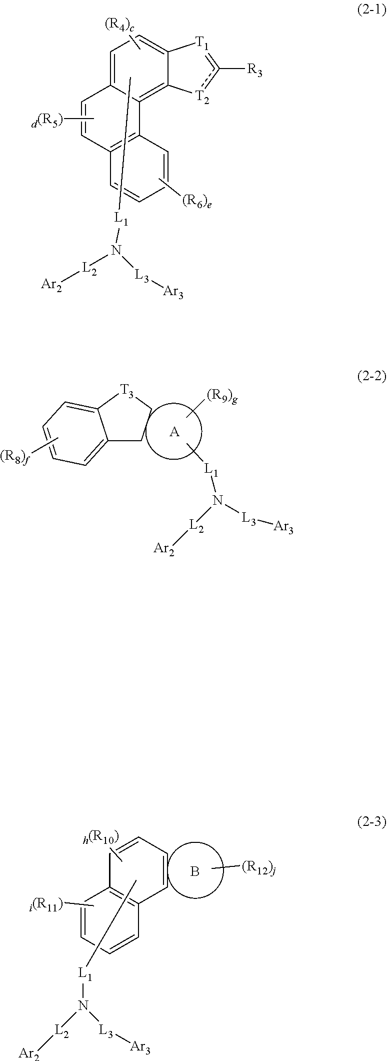
in Formulas 2-1 to 2-3,
T₁ and T₂ each independently represent —N═, —NR₇—, —O—, or —S—, provided that any one of T₁ and T₂ is —N═, and the other of T₁ and T₂ is —NR₇—, —O—, or —S—;
T₃ represents O or S;
ring A represents a substituted or unsubstituted phenanthrene;
ring B represents a substituted or unsubstituted benzene, or a substituted or unsubstituted naphthalene;
R₃ represents a substituted or unsubstituted (C₆-C₃₀)aryl, or a substituted or unsubstituted (3- to 30-membered)heteroaryl;
R₄ to R₁₂ each independently represent hydrogen, deuterium, a halogen, a cyano, a substituted or unsubstituted (C₁-C₃₀)alkyl, a substituted or unsubstituted (C₆-C₃₀)aryl, a substituted or unsubstituted (3- to 30-membered)heteroaryl, a substituted or unsubstituted (C₃-C₃₀)cycloalkyl, a substituted or unsubstituted (C₁-C₃₀)alkoxy, a substituted or unsubstituted tri(C₁-C₃₀)alkylsilyl, a substituted or unsubstituted di(C₁-C₃₀)alkyl(C₆-C₃₀)arylsilyl, a substituted or unsubstituted (C₁-C₃₀)alkyldi(C₆-C₃₀)arylsilyl, a substituted or unsubstituted tri(C₆-C₃₀)arylsilyl, a substituted or unsubstituted fused ring of (C₃-C₃₀) aliphatic ring and (C₆-C₃₀) aromatic ring, a substituted or unsubstituted mono- or di(C₁-C₃₀)alkylamino, a substituted or unsubstituted mono- or di(C₂-C₃₀)alkenylamino, a substituted or unsubstituted (C₁-C₃₀)alkyl(C₂-C₃₀)alkenylamino, a substituted or unsubstituted (C₁-C₃₀)alkyl(C₆-C₃₀)arylamino, a substituted or unsubstituted (C₁-C₃₀)alkyl(3- to 30-membered)heteroarylamino, a substituted or unsubstituted (C₂-C₃₀)alkenyl(C₆-C₃₀)arylamino, a substituted or unsubstituted (C₂-C₃₀)alkenyl(3- to 30-membered)heteroarylamino, a substituted or unsubstituted mono- or di(C₆-C₃₀)arylamino, a substituted or unsubstituted mono- or di(3- to 30-membered)heteroarylamino, or a substituted or unsubstituted (C₆-C₃₀)aryl(3- to 30-membered)heteroarylamino; or may be linked to the adjacent substituents to form a ring(s);
c, d, and h each independently represent an integer of 1 or 2, e, f, and i each independently represent an integer of 1 to 4, g represents an integer of 1 to 7, and j represents an integer of 1 to 6;
when c to j are an integer of 2 or more, each of R₄ to each of R₆ and each of R₈ to each of R₁₂ may be the same as or different from each other; and
L₁ to L₃, Ar₂, and Ar₃ are as defined in claim 1.

5. The plurality of host materials according to claim 4, wherein when L₃ in Formula 2-1 is a substituted or unsubstituted phenylene, Formula 2-1 is represented by the following Formula 2-1-a: 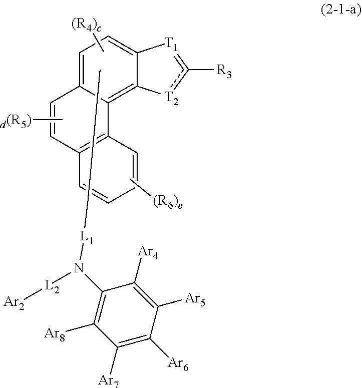
in Formula 2-1-a,
Ar₄ to Ar₈ each are as defined for Ar₃ in claim 1; provided that Ar₆ is not a substituted or unsubstituted heteroaryl; and
T₁, T₂, R₃ to R₆, and c to e each are as defined in claim 4, and L₁, L₂, and Ar₂ each are as defined in claim 1.

6. The plurality of host materials according to claim 4, wherein the compound represented by Formula 2-2 is represented by any one of the following Formulas 2-2a to 2-2c: 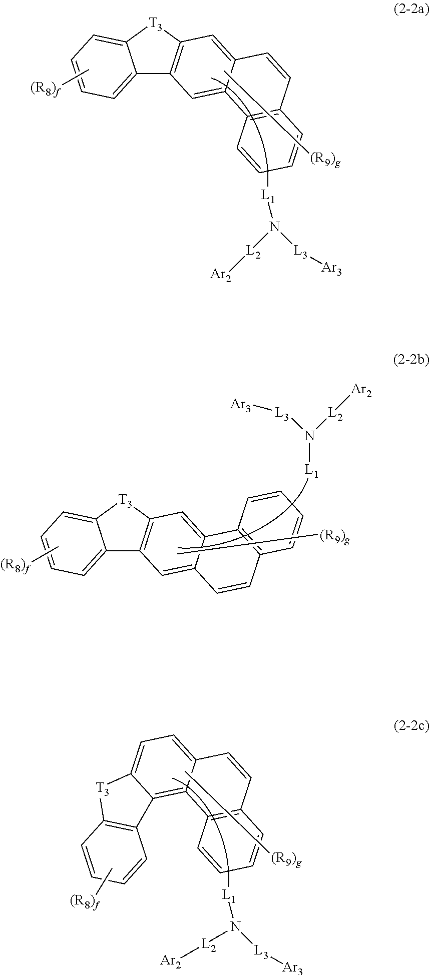
in Formulas 2-2a to 2-2c,
T₃, R₈, R₉, L₁ to L₃, Ar₂, Ar₃, f, and g are as defined in claim 4.

7. The plurality of host materials according to claim 4, wherein the compound represented by Formula 2-3 is represented by any one of the following Formulas 2-3a to 2-3d: 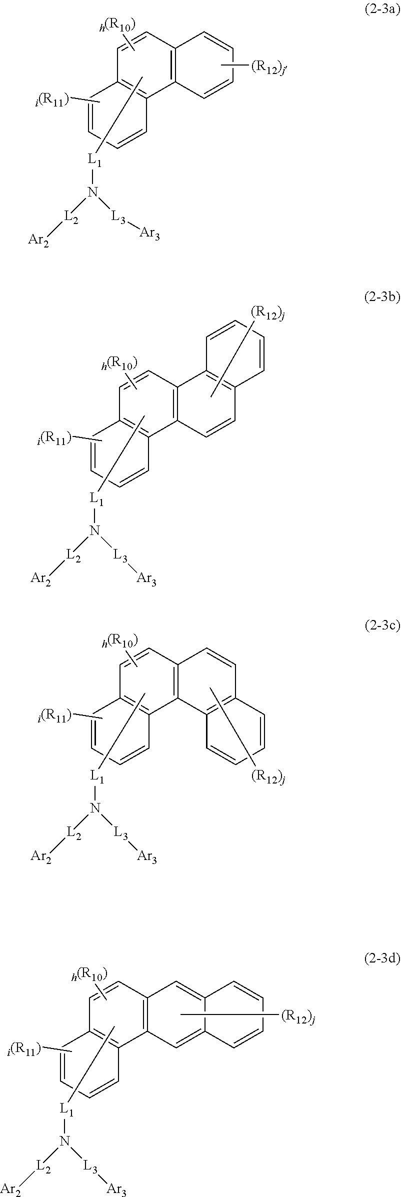
in Formulas 2-3a to 2-3d,
j′ is an integer of 1 to 4;
when j′ is an integer of 2 or more, each of R₁₂ may be the same as or different from each other; and
R₁₀ to R₁₂, L₁ to L₃, Ar₂, Ar₃, and h to j are as defined in claim 4.

8. The plurality of host materials according to claim 1, which further comprises a third host material comprising a compound represented by the following Formula 3: 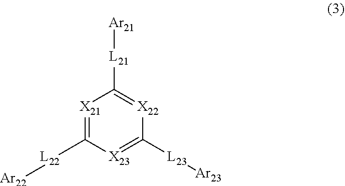
in Formula 3,
X₂₁ to X₂₃ each independently represent N or CR₂₀; provided that at least one of X₂₁ to X₂₃ is N;
R₂₀ represents hydrogen, deuterium, a halogen, a cyano, a substituted or unsubstituted (C₁-C₃₀)alkyl, a substituted or unsubstituted (C₆-C₃₀)aryl, a substituted or unsubstituted (3- to 30-membered)heteroaryl, a substituted or unsubstituted (C₃-C₃₀)cycloalkyl, a substituted or unsubstituted (C₁-C₃₀)alkoxy, a substituted or unsubstituted tri(C₁-C₃₀)alkylsilyl, a substituted or unsubstituted di(C₁-C₃₀)alkyl(C₆-C₃₀)arylsilyl, a substituted or unsubstituted (C₁-C₃₀)alkyldi(C₆-C₃₀)arylsilyl, a substituted or unsubstituted tri(C₆-C₃₀)arylsilyl, or a substituted or unsubstituted fused ring of (C₃-C₃₀) aliphatic ring and (C₆-C₃₀) aromatic ring;
L₂₁ to L₂₃ each independently represent a single bond, a substituted or unsubstituted (C₆-C₃₀)arylene, or a substituted or unsubstituted (3- to 30-membered)heteroarylene; and
Ar₂₁ to Ar₂₃ each independently represent a substituted or unsubstituted (C₁-C₃₀)alkyl, a substituted or unsubstituted (C₃-C₃₀)cycloalkyl, a substituted or unsubstituted (3- to 7-membered)heterocycloalkyl, a substituted or unsubstituted fused ring of (C₃-C₃₀) aliphatic ring and (C₆-C₃₀) aromatic ring, a substituted or unsubstituted (C₆-C₃₀)aryl, a substituted or unsubstituted (3- to 30-membered)heteroaryl, a substituted or unsubstituted tri(C₁-C₃₀)alkylsilyl, a substituted or unsubstituted di(C₁-C₃₀)alkyl(C₆-C₃₀)arylsilyl, a substituted or unsubstituted (C₁-C₃₀)alkyldi(C₆-C₃₀)arylsilyl, or a substituted or unsubstituted tri(C₆-C₃₀)arylsilyl.

9. The plurality of host materials according to claim 8, wherein Formula 3 is represented by any one of the following Formulas 3-1 to 3-4: 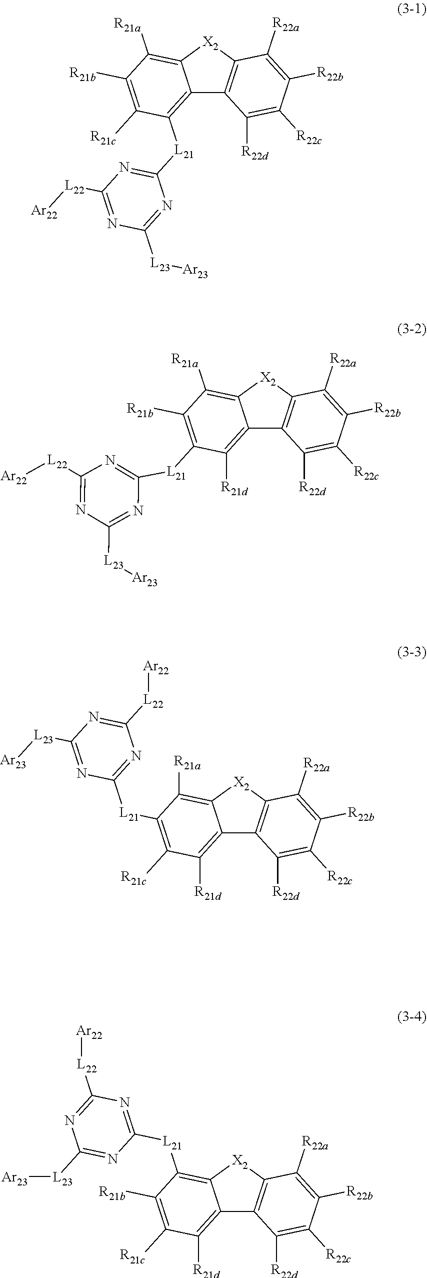
In Formulas 3-1 to 3-4,
X₂ represents O or S;
R₂₁ₐ to R_{21d} and R₂₂ₐ to R_{22d} each independently represent hydrogen, deuterium, a substituted or unsubstituted (C₆-C₃₀)aryl, or a substituted or unsubstituted (3- to 30-membered)heteroaryl; and
L₂₁ to L₂₃, Ar₂₂, and Ar₂₃ are as defined in claim 1.

10. The plurality of host materials according to claim 1, wherein the compound represented by Formula 1 is selected from the following compounds:

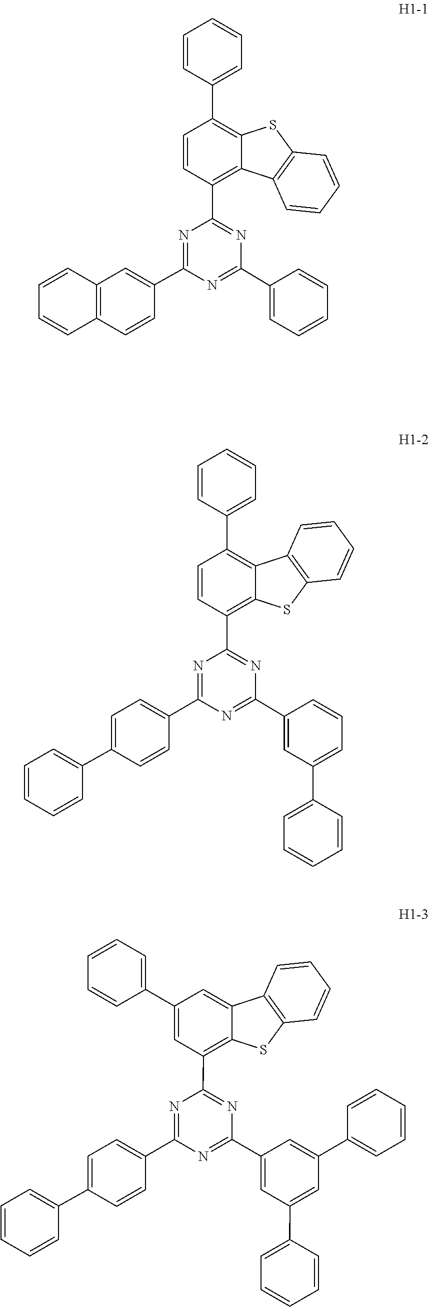 [SMILES: c1ccc(-c2nc(-c3ccc4ccccc4c3)nc(-c3ccc(-c4ccccc4)c4sc5ccccc5c34)n2)cc1 ; c1ccc(-c2ccc(-c3nc(-c4cccc(-c5ccccc5)c4)nc(-c4ccc(-c5ccccc5)c5c4sc4ccccc45)n3)cc2)cc1 ; c1ccc(-c2ccc(-c3nc(-c4cc(-c5ccccc5)cc(-c5ccccc5)c4)nc(-c4cc(-c5ccccc5)cc5c4sc4ccccc45)n3)cc2)cc1]

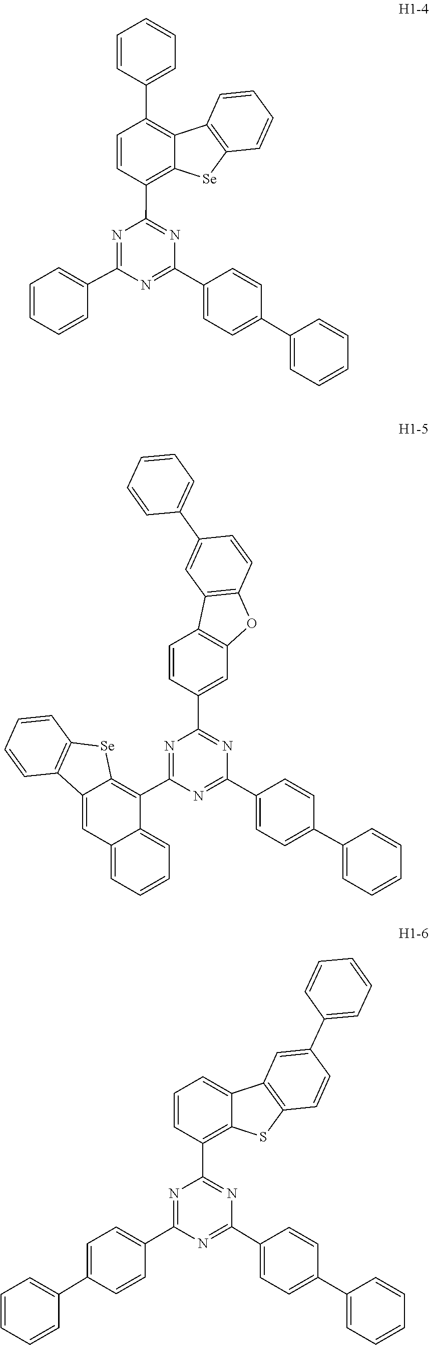 [SMILES: c1ccc(-c2ccc(-c3nc(-c4ccccc4)nc(-c4ccc(-c5ccccc5)c5c4[se]c4ccccc45)n3)cc2)cc1 ; c1ccc(-c2ccc(-c3nc(-c4ccc5c(c4)oc4ccc(-c6ccccc6)cc45)nc(-c4c5ccccc5cc5c4[se]c4ccccc45)n3)cc2)cc1 ; c1ccc(-c2ccc(-c3nc(-c4ccc(-c5ccccc5)cc4)nc(-c4cccc5c4sc4ccc(-c6ccccc6)cc45)n3)cc2)cc1]

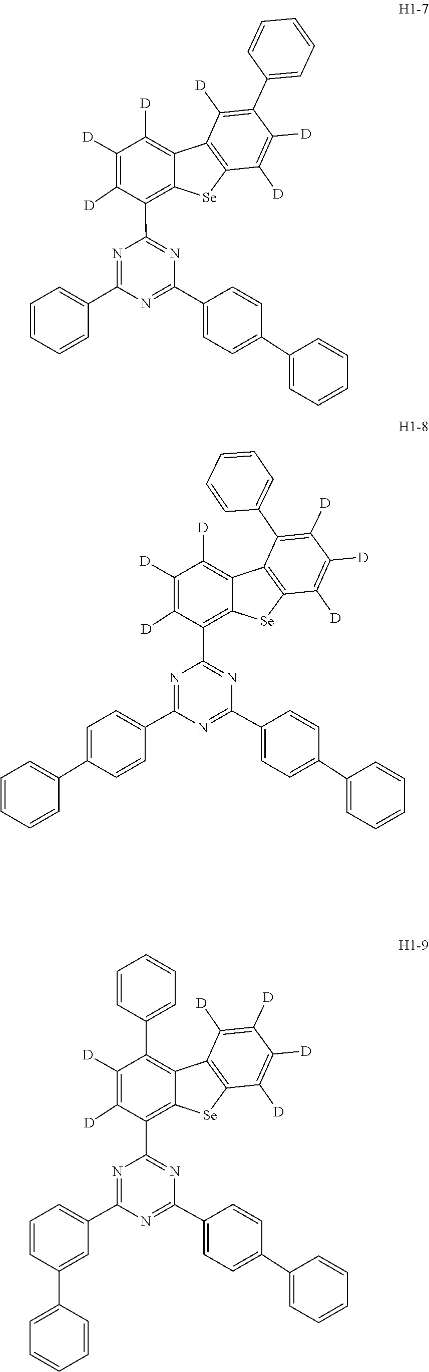 [SMILES: [2H]c1c(-c2ccccc2)c([2H])c2c([se]c3c(-c4nc(-c5ccccc5)nc(-c5ccc(-c6ccccc6)cc5)n4)c([2H])c([2H])c([2H])c32)c1[2H] ; [2H]c1c([2H])c(-c2ccccc2)c2c([se]c3c(-c4nc(-c5ccc(-c6ccccc6)cc5)nc(-c5ccc(-c6ccccc6)cc5)n4)c([2H])c([2H])c([2H])c32)c1[2H] ; [2H]c1c([2H])c([2H])c2c([se]c3c(-c4nc(-c5ccc(-c6ccccc6)cc5)nc(-c5cccc(-c6ccccc6)c5)n4)c([2H])c([2H])c(-c4ccccc4)c32)c1[2H]]

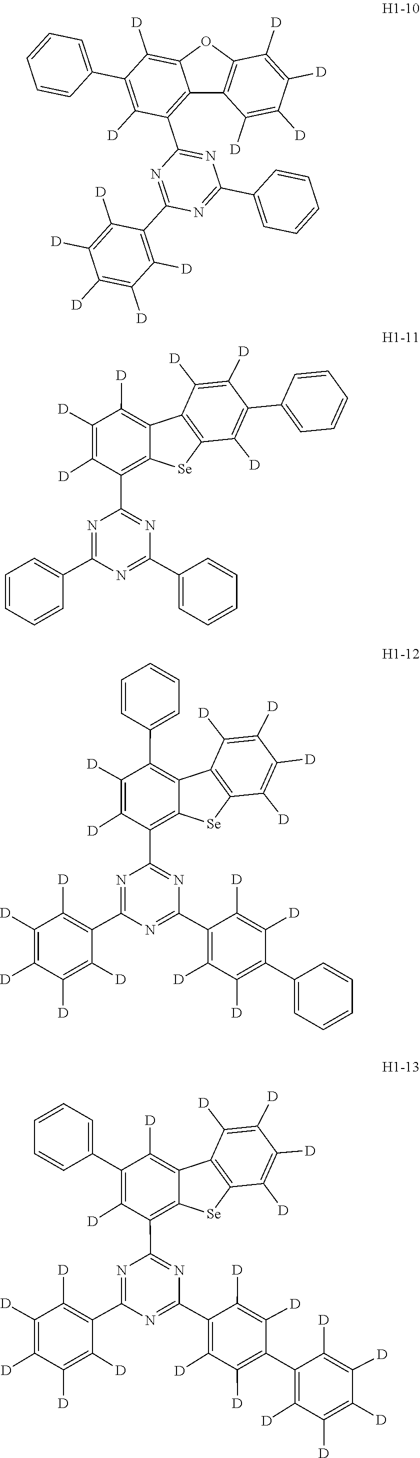

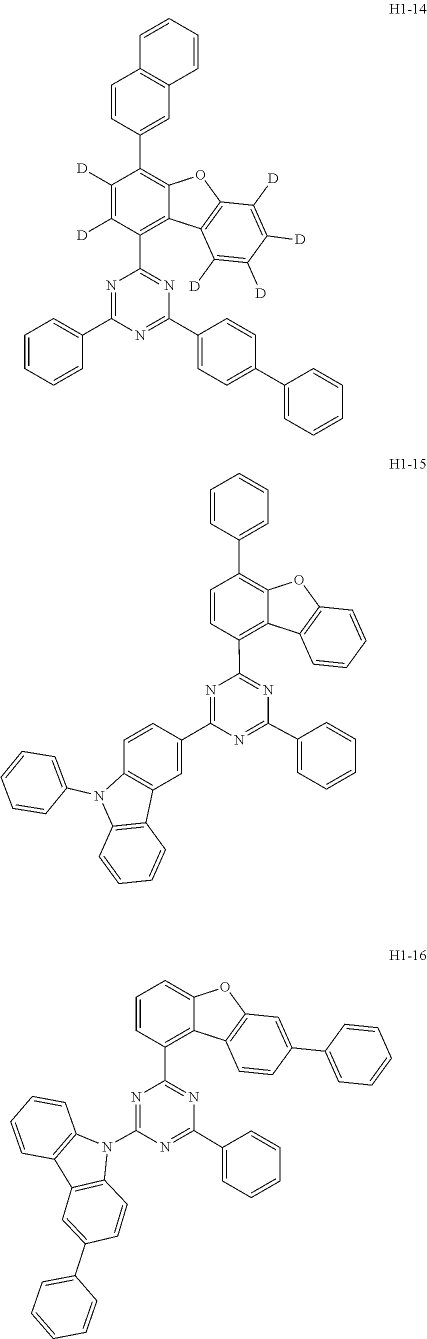 [SMILES: [2H]c1c([2H])c([2H])c2c(oc3c(-c4ccc5ccccc5c4)c([2H])c([2H])c(-c4nc(-c5ccccc5)nc(-c5ccc(-c6ccccc6)cc5)n4)c32)c1[2H] ; c1ccc(-c2nc(-c3ccc4c(c3)c3ccccc3n4-c3ccccc3)nc(-c3ccc(-c4ccccc4)c4oc5ccccc5c34)n2)cc1 ; c1ccc(-c2ccc3c(c2)oc2cccc(-c4nc(-c5ccccc5)nc(-n5c6ccccc6c6cc(-c7ccccc7)ccc65)n4)c23)cc1]

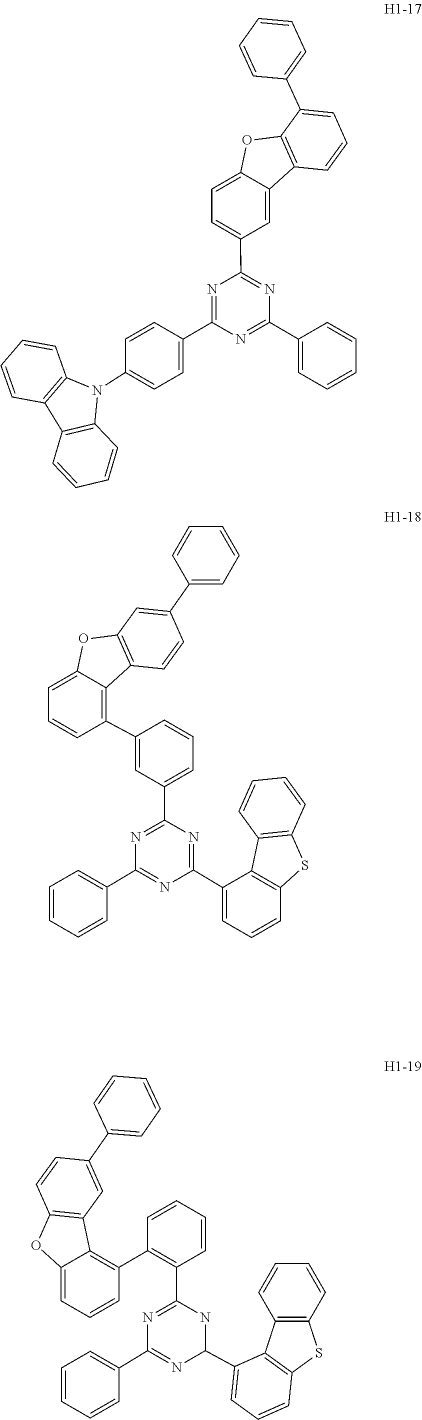

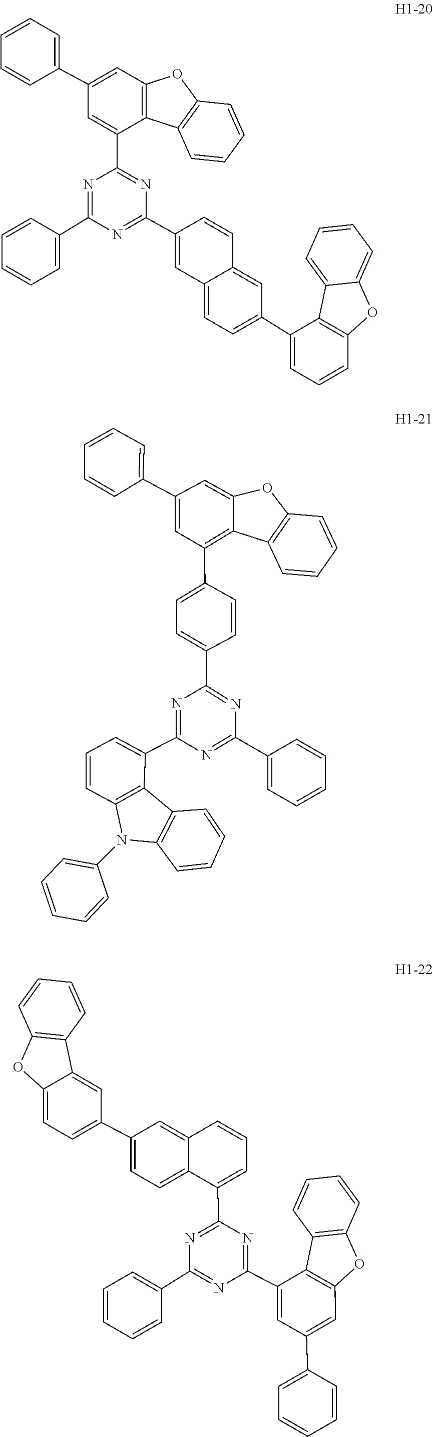 [SMILES: c1ccc(-c2cc(-c3nc(-c4ccccc4)nc(-c4ccc5cc(-c6cccc7oc8ccccc8c67)ccc5c4)n3)c3c(c2)oc2ccccc23)cc1 ; c1ccc(-c2cc(-c3ccc(-c4nc(-c5ccccc5)nc(-c5cccc6c5c5ccccc5n6-c5ccccc5)n4)cc3)c3c(c2)oc2ccccc23)cc1 ; c1ccc(-c2cc(-c3nc(-c4ccccc4)nc(-c4cccc5cc(-c6ccc7oc8ccccc8c7c6)ccc45)n3)c3c(c2)oc2ccccc23)cc1]

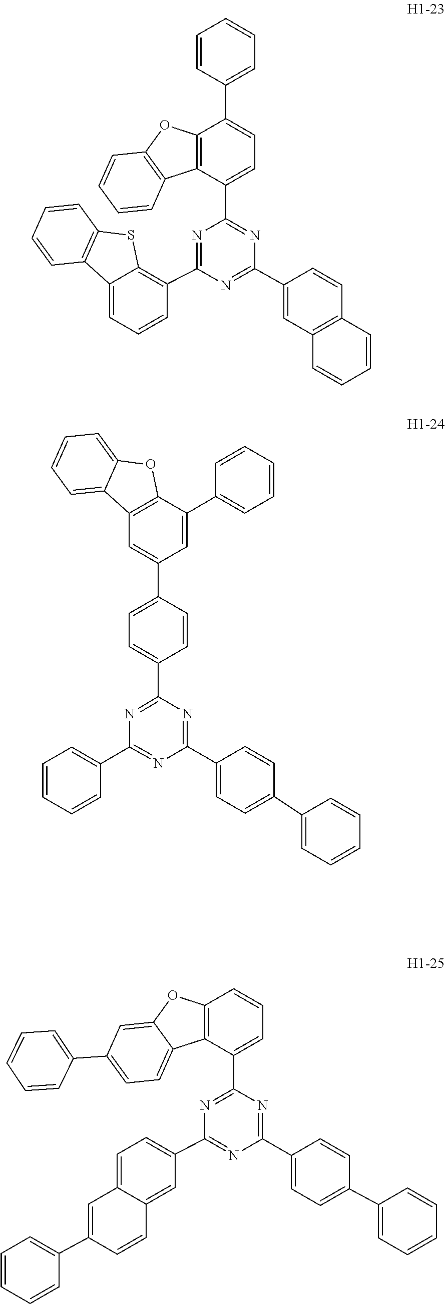

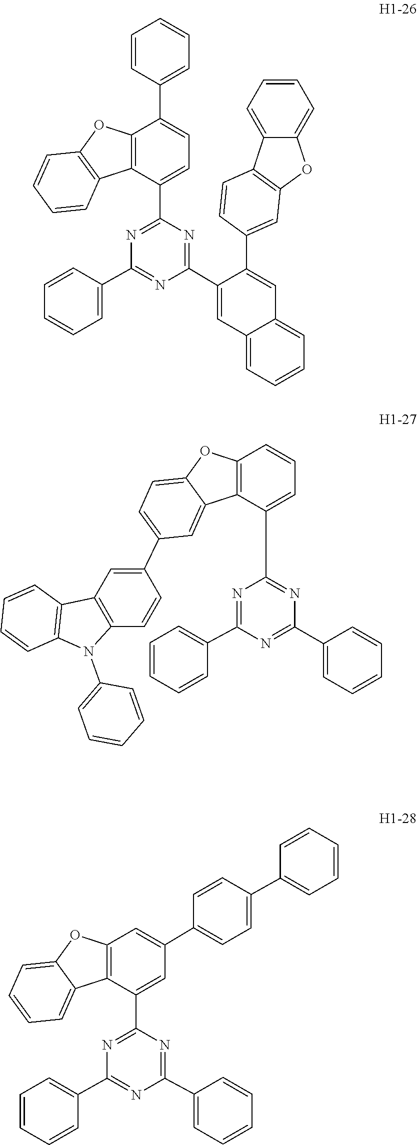 [SMILES: c1ccc(-c2nc(-c3cc4ccccc4cc3-c3ccc4c(c3)oc3ccccc34)nc(-c3ccc(-c4ccccc4)c4oc5ccccc5c34)n2)cc1 ; c1ccc(-c2nc(-c3ccccc3)nc(-c3cccc4oc5ccc(-c6ccc7c(c6)c6ccccc6n7-c6ccccc6)cc5c34)n2)cc1 ; c1ccc(-c2ccc(-c3cc(-c4nc(-c5ccccc5)nc(-c5ccccc5)n4)c4c(c3)oc3ccccc34)cc2)cc1]

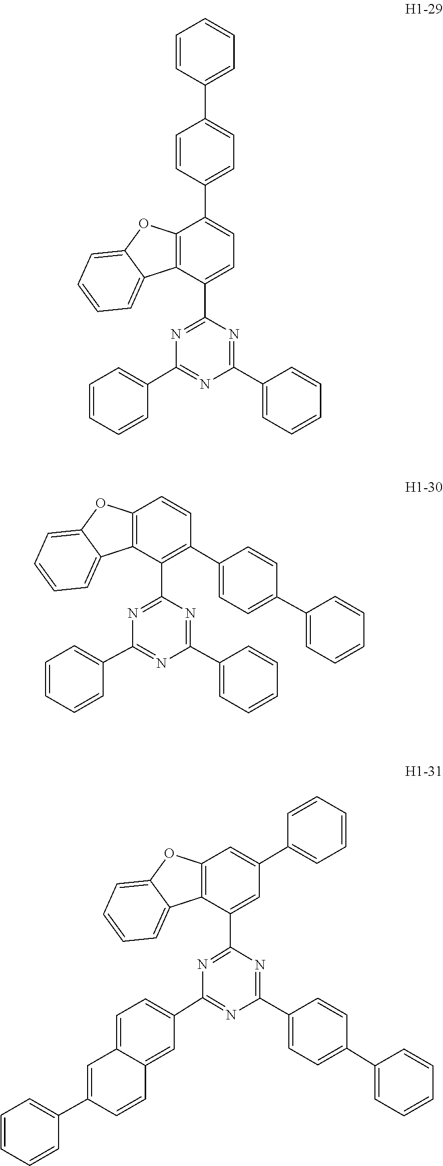 [SMILES: c1ccc(-c2ccc(-c3ccc(-c4nc(-c5ccccc5)nc(-c5ccccc5)n4)c4c3oc3ccccc34)cc2)cc1 ; c1ccc(-c2ccc(-c3ccc4oc5ccccc5c4c3-c3nc(-c4ccccc4)nc(-c4ccccc4)n3)cc2)cc1 ; c1ccc(-c2ccc(-c3nc(-c4ccc5cc(-c6ccccc6)ccc5c4)nc(-c4cc(-c5ccccc5)cc5oc6ccccc6c45)n3)cc2)cc1]

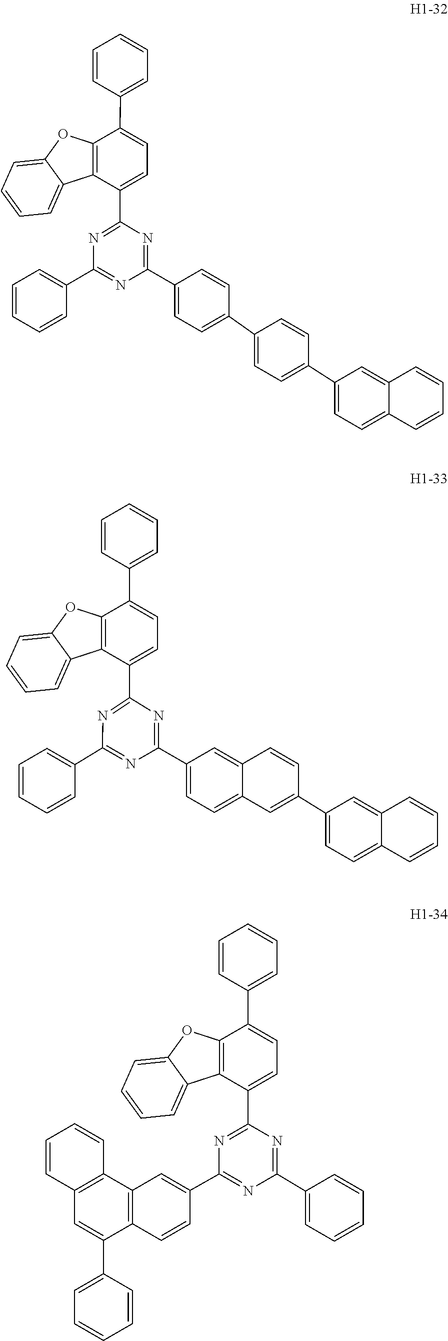

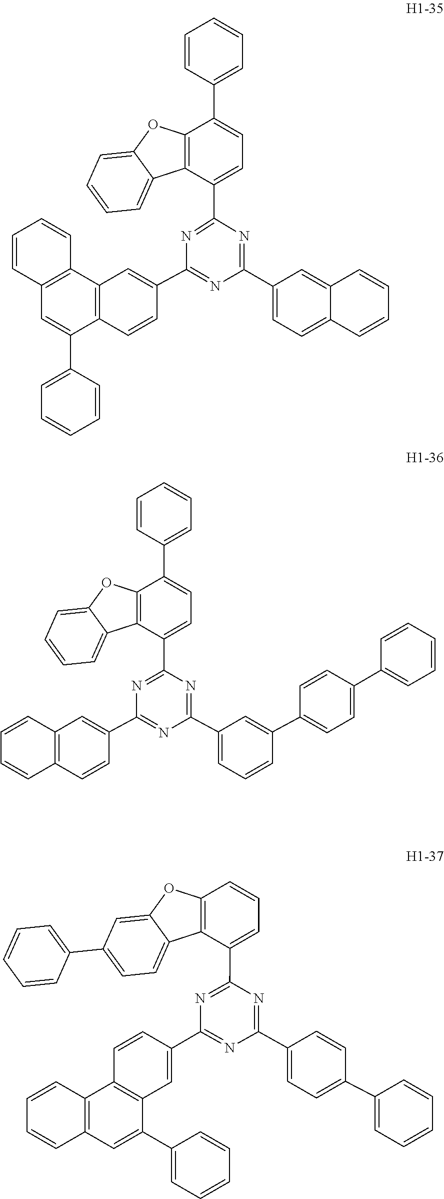

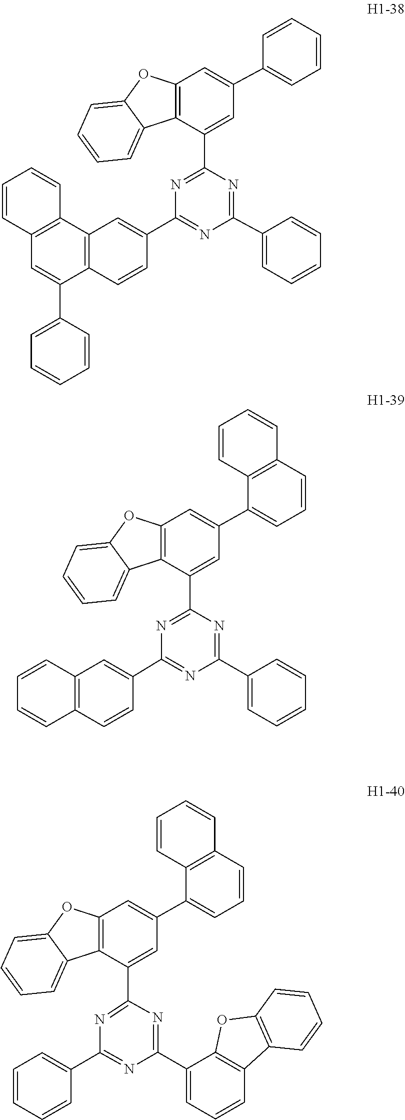

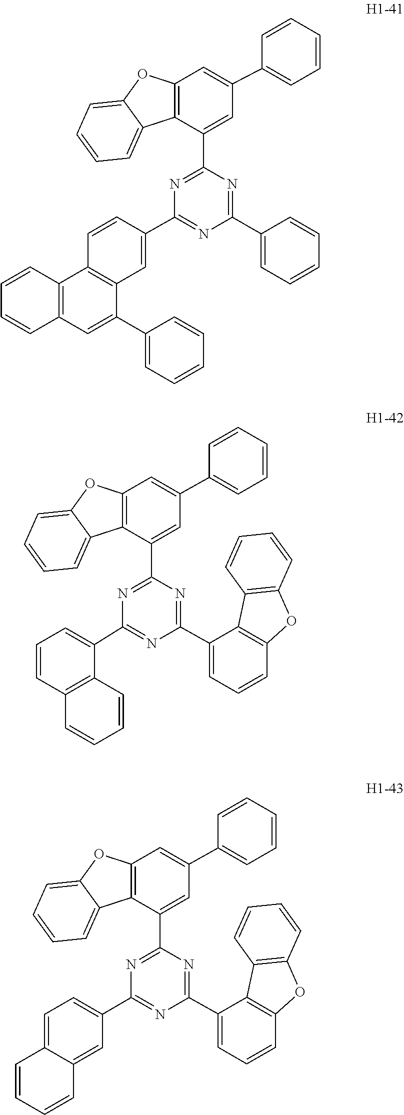 [SMILES: c1ccc(-c2cc(-c3nc(-c4ccccc4)nc(-c4ccc5c(c4)c(-c4ccccc4)cc4ccccc45)n3)c3c(c2)oc2ccccc23)cc1 ; c1ccc(-c2cc(-c3nc(-c4cccc5ccccc45)nc(-c4cccc5oc6ccccc6c45)n3)c3c(c2)oc2ccccc23)cc1 ; c1ccc(-c2cc(-c3nc(-c4ccc5ccccc5c4)nc(-c4cccc5oc6ccccc6c45)n3)c3c(c2)oc2ccccc23)cc1]

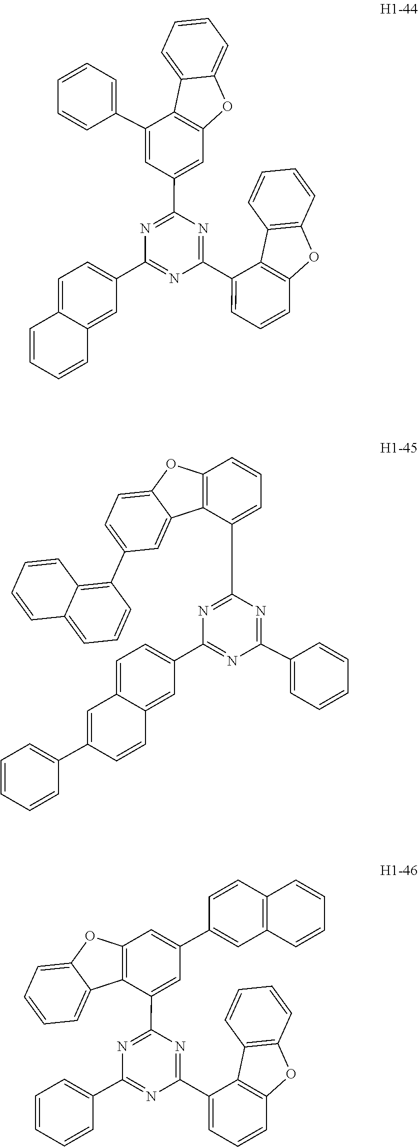 [SMILES: c1ccc(-c2cc(-c3nc(-c4ccc5ccccc5c4)nc(-c4cccc5oc6ccccc6c45)n3)cc3oc4ccccc4c23)cc1 ; c1ccc(-c2ccc3cc(-c4nc(-c5ccccc5)nc(-c5cccc6oc7ccc(-c8cccc9ccccc89)cc7c56)n4)ccc3c2)cc1 ; c1ccc(-c2nc(-c3cccc4oc5ccccc5c34)nc(-c3cc(-c4ccc5ccccc5c4)cc4oc5ccccc5c34)n2)cc1]

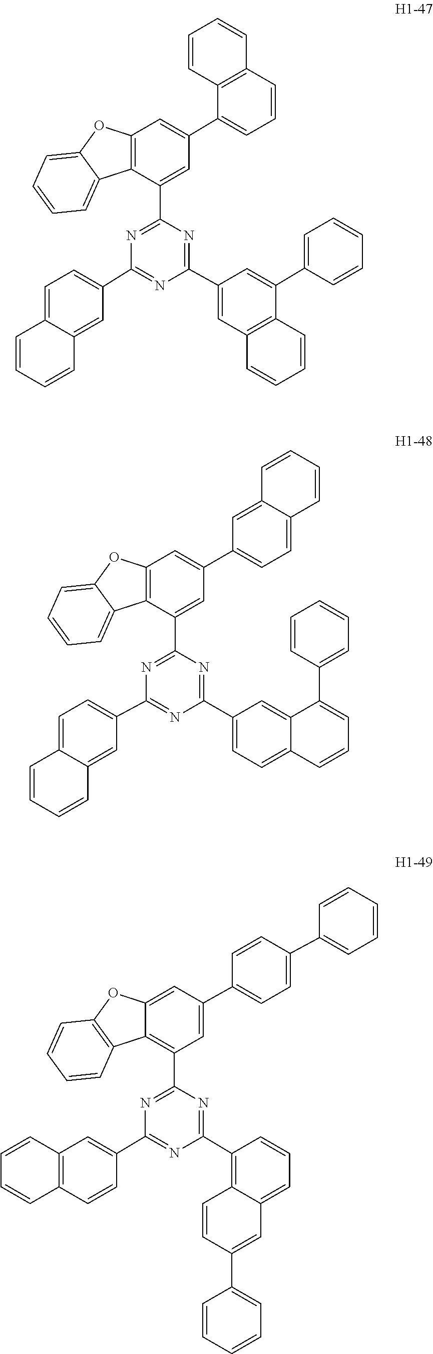 [SMILES: c1ccc(-c2cc(-c3nc(-c4ccc5ccccc5c4)nc(-c4cc(-c5cccc6ccccc56)cc5oc6ccccc6c45)n3)cc3ccccc23)cc1 ; c1ccc(-c2cccc3ccc(-c4nc(-c5ccc6ccccc6c5)nc(-c5cc(-c6ccc7ccccc7c6)cc6oc7ccccc7c56)n4)cc23)cc1 ; c1ccc(-c2ccc(-c3cc(-c4nc(-c5ccc6ccccc6c5)nc(-c5cccc6cc(-c7ccccc7)ccc56)n4)c4c(c3)oc3ccccc34)cc2)cc1]

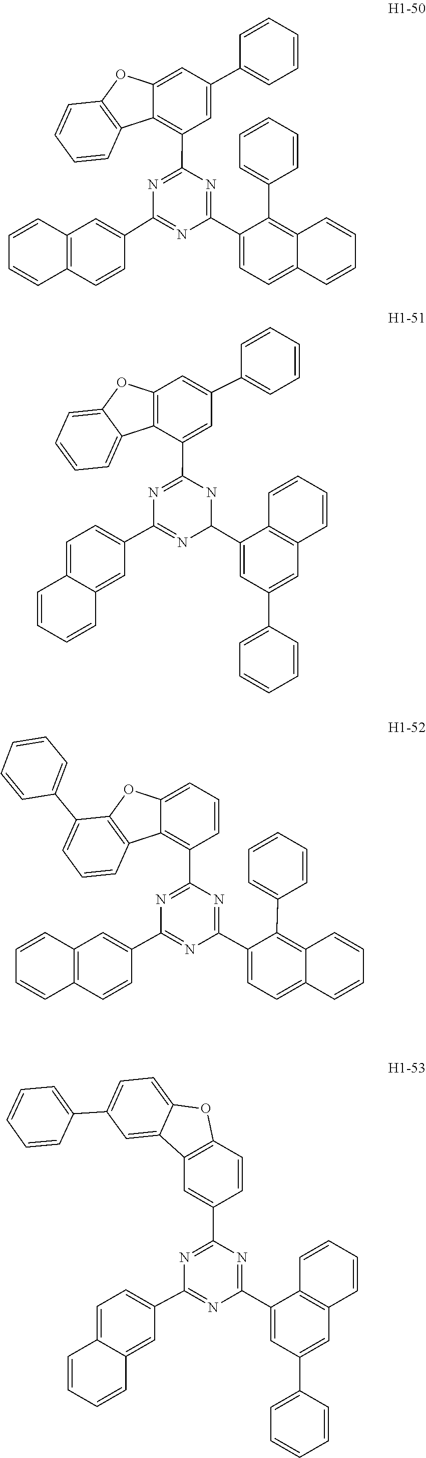

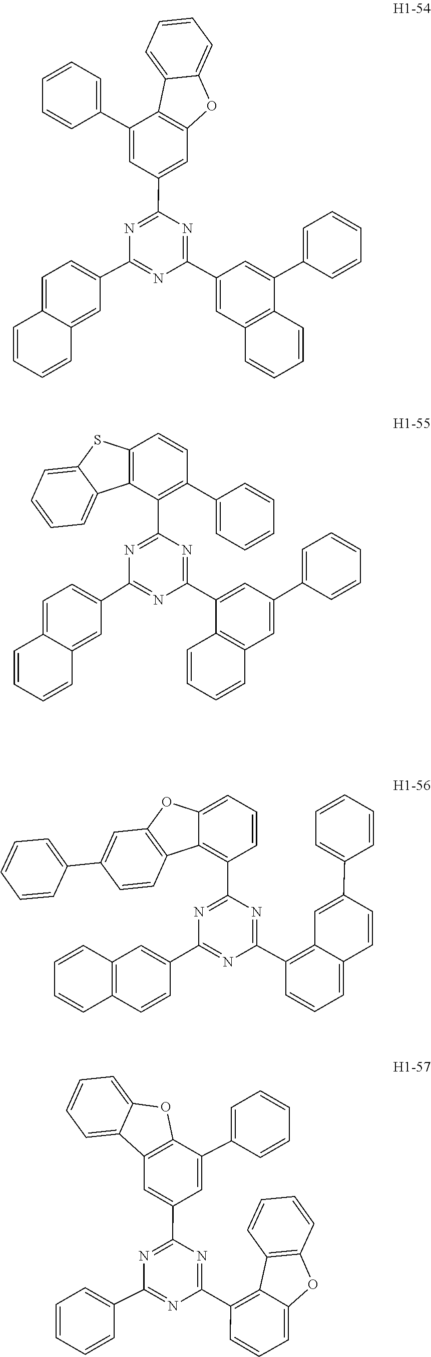 [SMILES: c1ccc(-c2cc(-c3nc(-c4ccc5ccccc5c4)nc(-c4cc(-c5ccccc5)c5c(c4)oc4ccccc45)n3)cc3ccccc23)cc1 ; c1ccc(-c2cc(-c3nc(-c4ccc5ccccc5c4)nc(-c4c(-c5ccccc5)ccc5sc6ccccc6c45)n3)c3ccccc3c2)cc1 ; c1ccc(-c2ccc3c(c2)oc2cccc(-c4nc(-c5ccc6ccccc6c5)nc(-c5cccc6ccc(-c7ccccc7)cc56)n4)c23)cc1 ; c1ccc(-c2nc(-c3cc(-c4ccccc4)c4oc5ccccc5c4c3)nc(-c3cccc4oc5ccccc5c34)n2)cc1]

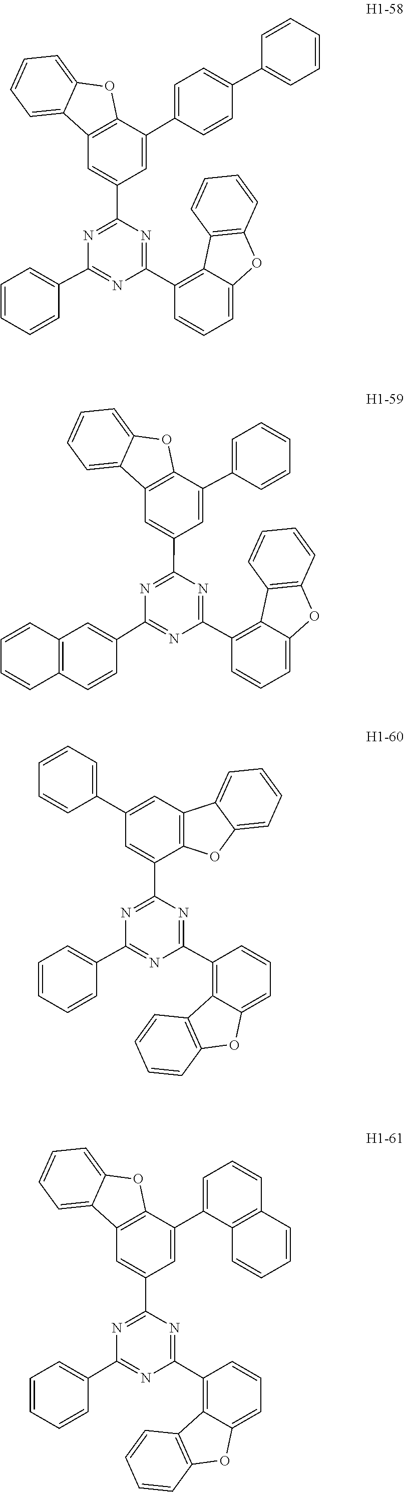 [SMILES: c1ccc(-c2ccc(-c3cc(-c4nc(-c5ccccc5)nc(-c5cccc6oc7ccccc7c56)n4)cc4c3oc3ccccc34)cc2)cc1 ; c1ccc(-c2cc(-c3nc(-c4ccc5ccccc5c4)nc(-c4cccc5oc6ccccc6c45)n3)cc3c2oc2ccccc23)cc1 ; c1ccc(-c2cc(-c3nc(-c4ccccc4)nc(-c4cccc5oc6ccccc6c45)n3)c3oc4ccccc4c3c2)cc1 ; c1ccc(-c2nc(-c3cc(-c4cccc5ccccc45)c4oc5ccccc5c4c3)nc(-c3cccc4oc5ccccc5c34)n2)cc1]

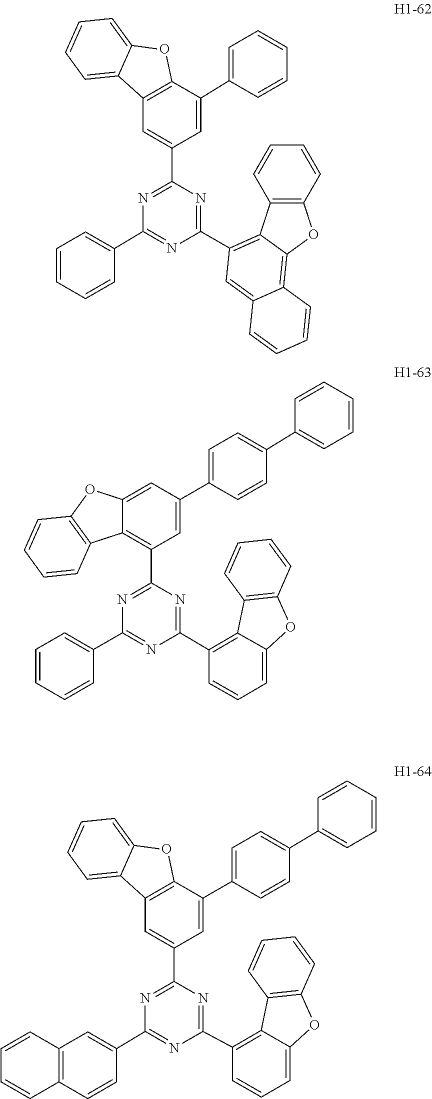

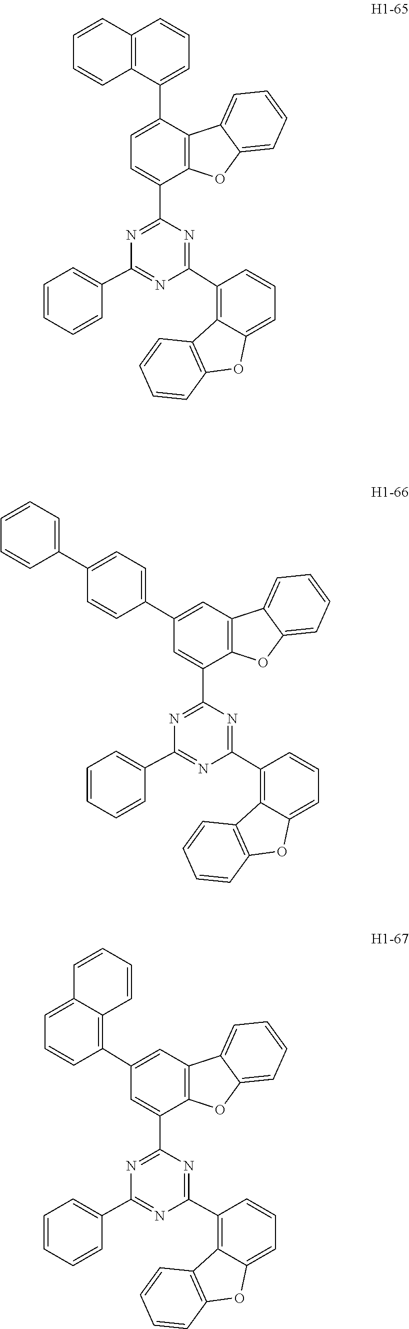 [SMILES: c1ccc(-c2nc(-c3ccc(-c4cccc5ccccc45)c4c3oc3ccccc34)nc(-c3cccc4oc5ccccc5c34)n2)cc1 ; c1ccc(-c2ccc(-c3cc(-c4nc(-c5ccccc5)nc(-c5cccc6oc7ccccc7c56)n4)c4oc5ccccc5c4c3)cc2)cc1 ; c1ccc(-c2nc(-c3cc(-c4cccc5ccccc45)cc4c3oc3ccccc34)nc(-c3cccc4oc5ccccc5c34)n2)cc1]

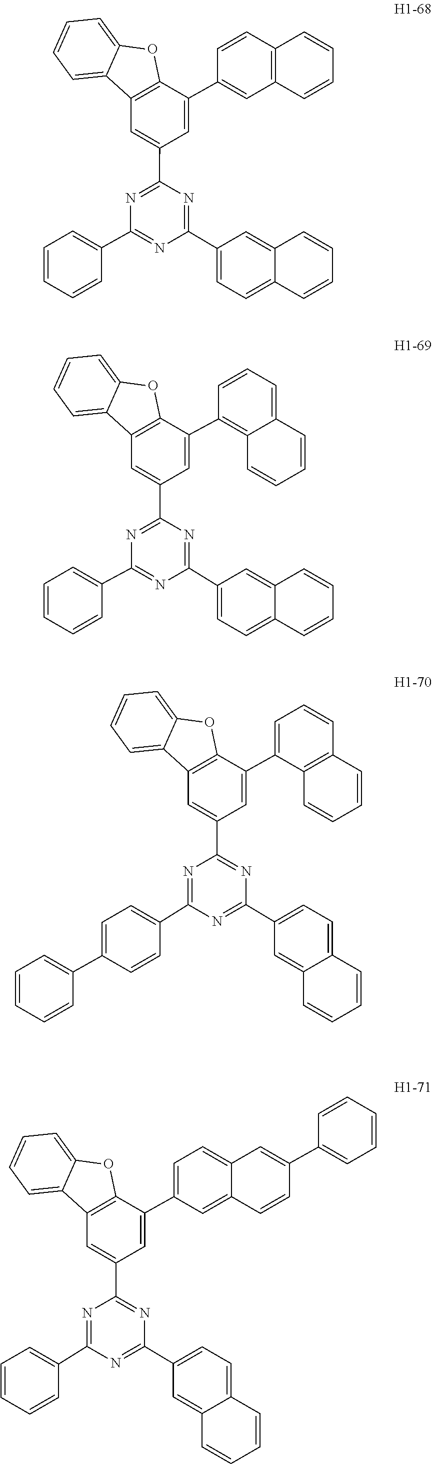 [SMILES: c1ccc(-c2nc(-c3ccc4ccccc4c3)nc(-c3cc(-c4ccc5ccccc5c4)c4oc5ccccc5c4c3)n2)cc1 ; c1ccc(-c2nc(-c3ccc4ccccc4c3)nc(-c3cc(-c4cccc5ccccc45)c4oc5ccccc5c4c3)n2)cc1 ; c1ccc(-c2ccc(-c3nc(-c4ccc5ccccc5c4)nc(-c4cc(-c5cccc6ccccc56)c5oc6ccccc6c5c4)n3)cc2)cc1 ; c1ccc(-c2ccc3cc(-c4cc(-c5nc(-c6ccccc6)nc(-c6ccc7ccccc7c6)n5)cc5c4oc4ccccc45)ccc3c2)cc1]

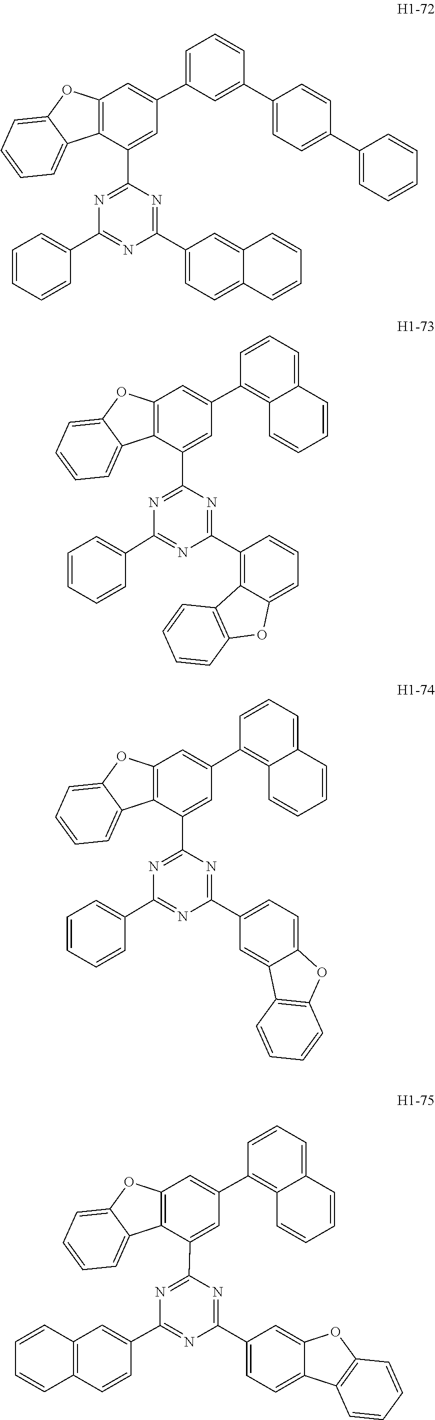

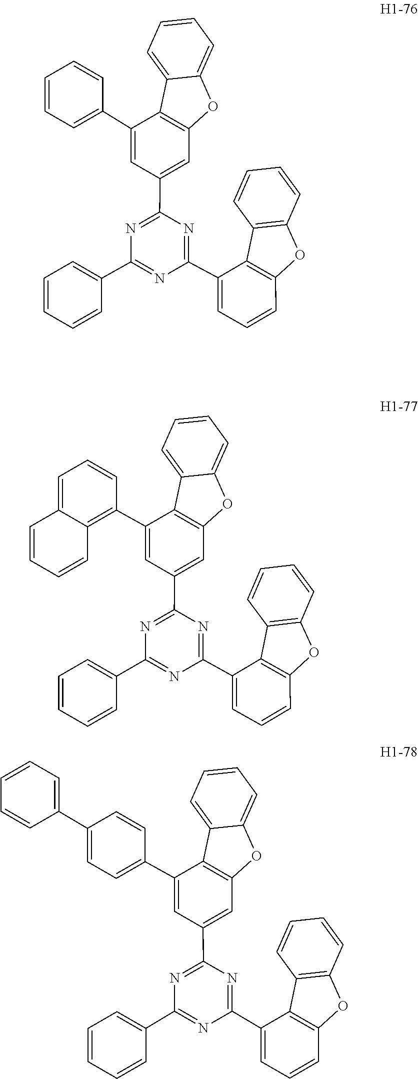 [SMILES: c1ccc(-c2nc(-c3cc(-c4ccccc4)c4c(c3)oc3ccccc34)nc(-c3cccc4oc5ccccc5c34)n2)cc1 ; c1ccc(-c2nc(-c3cc(-c4cccc5ccccc45)c4c(c3)oc3ccccc34)nc(-c3cccc4oc5ccccc5c34)n2)cc1 ; c1ccc(-c2ccc(-c3cc(-c4nc(-c5ccccc5)nc(-c5cccc6oc7ccccc7c56)n4)cc4oc5ccccc5c34)cc2)cc1]

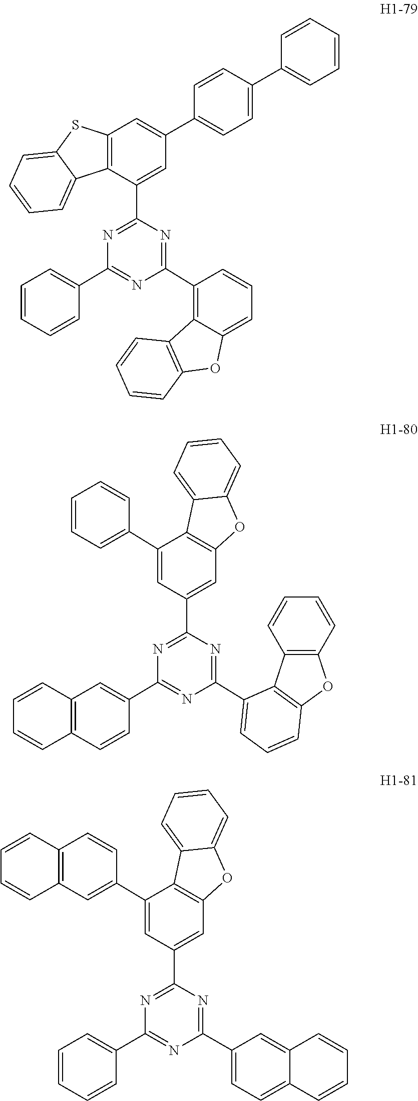 [SMILES: c1ccc(-c2ccc(-c3cc(-c4nc(-c5ccccc5)nc(-c5cccc6oc7ccccc7c56)n4)c4c(c3)sc3ccccc34)cc2)cc1 ; c1ccc(-c2cc(-c3nc(-c4ccc5ccccc5c4)nc(-c4cccc5oc6ccccc6c45)n3)cc3oc4ccccc4c23)cc1 ; c1ccc(-c2nc(-c3ccc4ccccc4c3)nc(-c3cc(-c4ccc5ccccc5c4)c4c(c3)oc3ccccc34)n2)cc1]

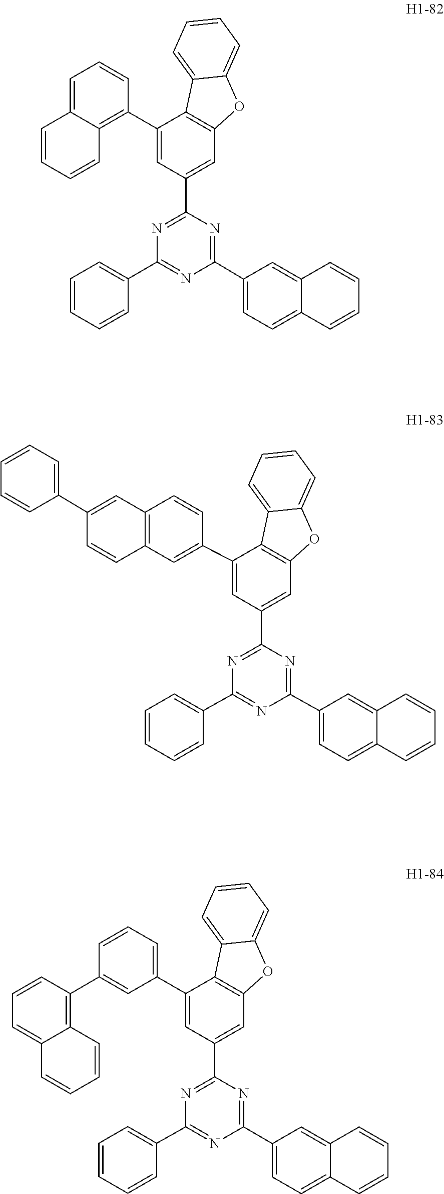 [SMILES: c1ccc(-c2nc(-c3ccc4ccccc4c3)nc(-c3cc(-c4cccc5ccccc45)c4c(c3)oc3ccccc34)n2)cc1 ; c1ccc(-c2ccc3cc(-c4cc(-c5nc(-c6ccccc6)nc(-c6ccc7ccccc7c6)n5)cc5oc6ccccc6c45)ccc3c2)cc1 ; c1ccc(-c2nc(-c3ccc4ccccc4c3)nc(-c3cc(-c4cccc(-c5cccc6ccccc56)c4)c4c(c3)oc3ccccc34)n2)cc1]

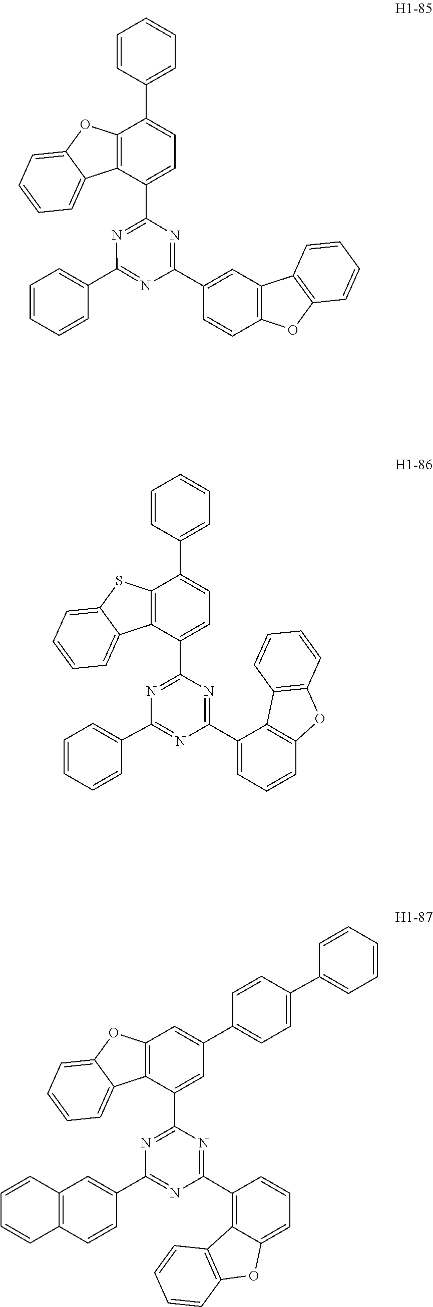

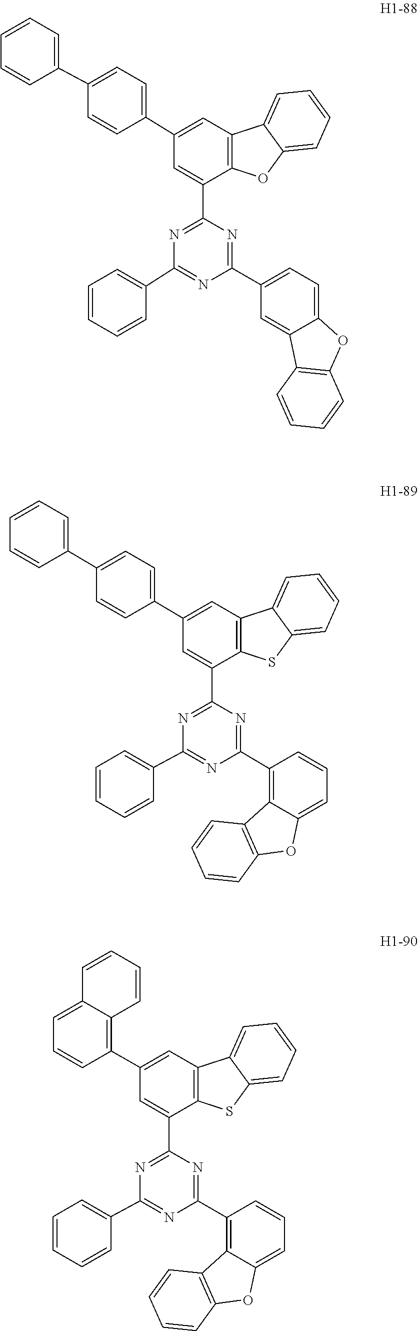 [SMILES: c1ccc(-c2ccc(-c3cc(-c4nc(-c5ccccc5)nc(-c5ccc6oc7ccccc7c6c5)n4)c4oc5ccccc5c4c3)cc2)cc1 ; c1ccc(-c2ccc(-c3cc(-c4nc(-c5ccccc5)nc(-c5cccc6oc7ccccc7c56)n4)c4sc5ccccc5c4c3)cc2)cc1 ; c1ccc(-c2nc(-c3cc(-c4cccc5ccccc45)cc4c3sc3ccccc34)nc(-c3cccc4oc5ccccc5c34)n2)cc1]

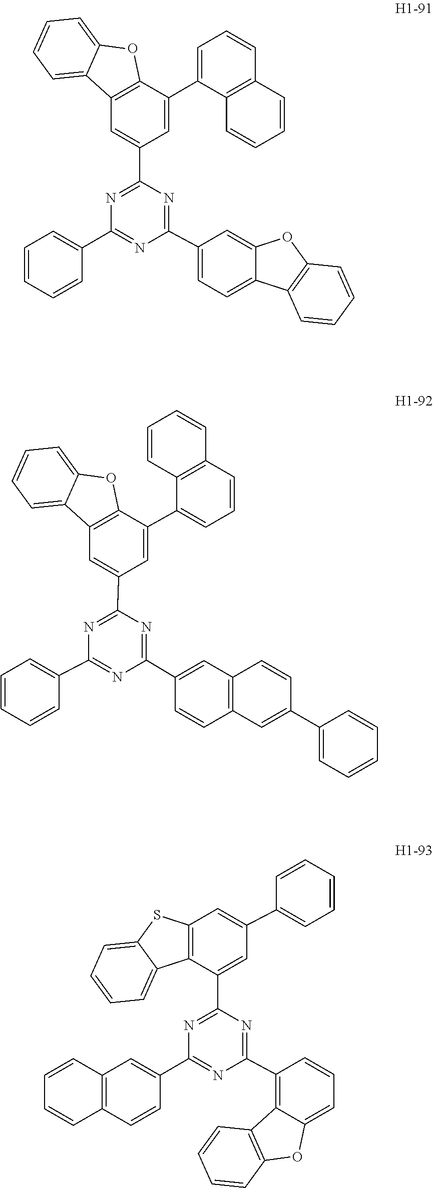 [SMILES: c1ccc(-c2nc(-c3ccc4c(c3)oc3ccccc34)nc(-c3cc(-c4cccc5ccccc45)c4oc5ccccc5c4c3)n2)cc1 ; c1ccc(-c2ccc3cc(-c4nc(-c5ccccc5)nc(-c5cc(-c6cccc7ccccc67)c6oc7ccccc7c6c5)n4)ccc3c2)cc1 ; c1ccc(-c2cc(-c3nc(-c4ccc5ccccc5c4)nc(-c4cccc5oc6ccccc6c45)n3)c3c(c2)sc2ccccc23)cc1]

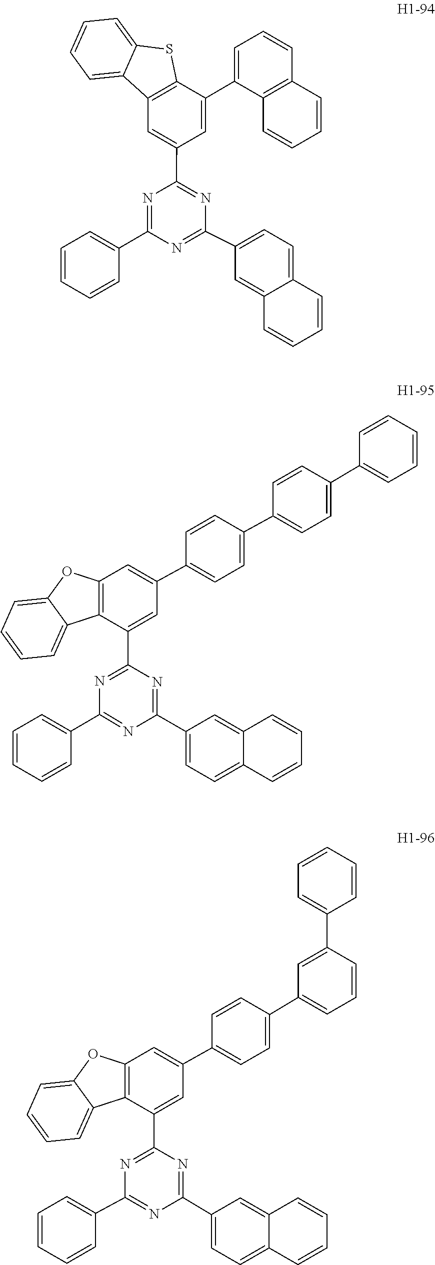

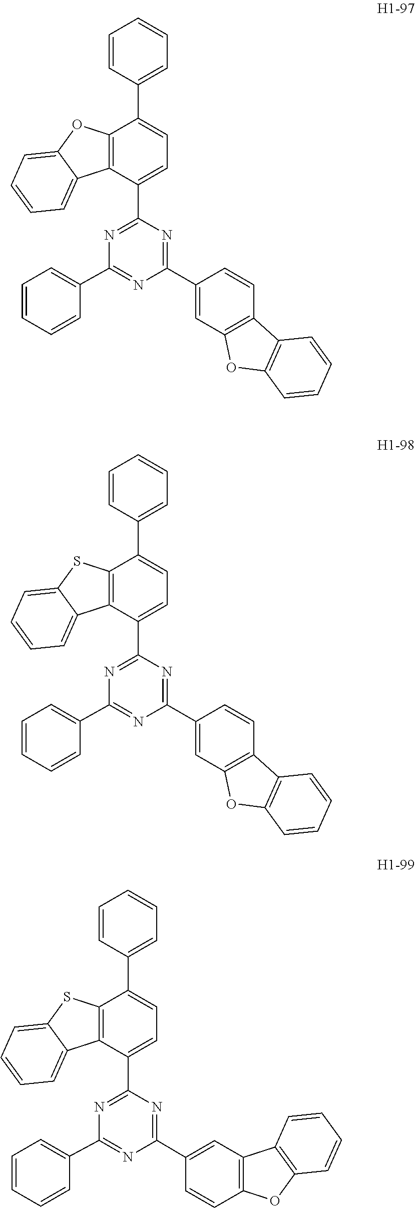 [SMILES: c1ccc(-c2nc(-c3ccc4c(c3)oc3ccccc34)nc(-c3ccc(-c4ccccc4)c4oc5ccccc5c34)n2)cc1 ; c1ccc(-c2nc(-c3ccc4c(c3)oc3ccccc34)nc(-c3ccc(-c4ccccc4)c4sc5ccccc5c34)n2)cc1 ; c1ccc(-c2nc(-c3ccc4oc5ccccc5c4c3)nc(-c3ccc(-c4ccccc4)c4sc5ccccc5c34)n2)cc1]

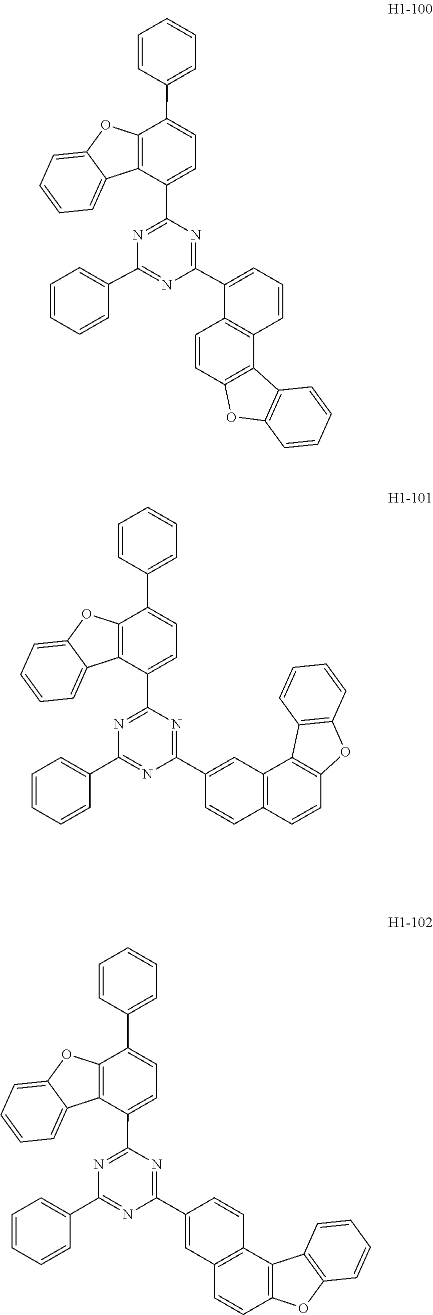 [SMILES: c1ccc(-c2nc(-c3cccc4c3ccc3oc5ccccc5c34)nc(-c3ccc(-c4ccccc4)c4oc5ccccc5c34)n2)cc1 ; c1ccc(-c2nc(-c3ccc4ccc5oc6ccccc6c5c4c3)nc(-c3ccc(-c4ccccc4)c4oc5ccccc5c34)n2)cc1 ; c1ccc(-c2nc(-c3ccc4c(ccc5oc6ccccc6c54)c3)nc(-c3ccc(-c4ccccc4)c4oc5ccccc5c34)n2)cc1]

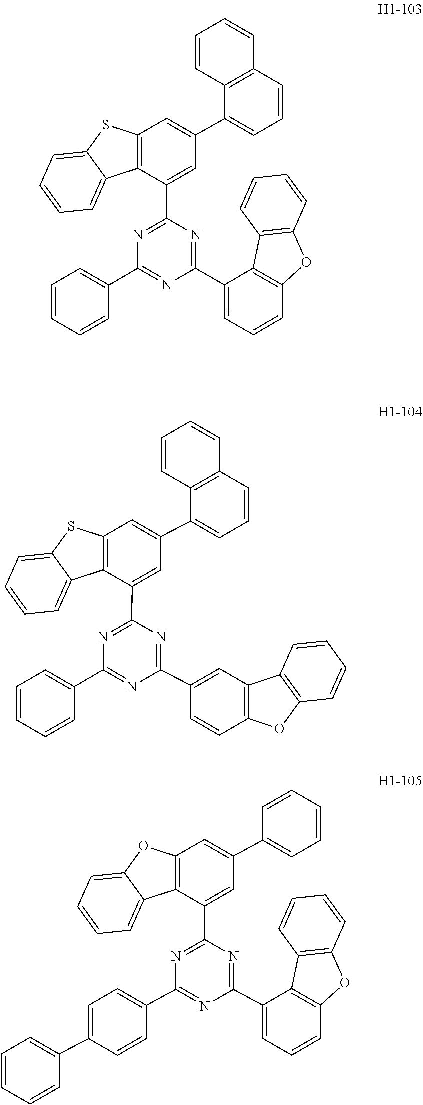 [SMILES: c1ccc(-c2nc(-c3cccc4oc5ccccc5c34)nc(-c3cc(-c4cccc5ccccc45)cc4sc5ccccc5c34)n2)cc1 ; c1ccc(-c2nc(-c3ccc4oc5ccccc5c4c3)nc(-c3cc(-c4cccc5ccccc45)cc4sc5ccccc5c34)n2)cc1 ; c1ccc(-c2ccc(-c3nc(-c4cccc5oc6ccccc6c45)nc(-c4cc(-c5ccccc5)cc5oc6ccccc6c45)n3)cc2)cc1]

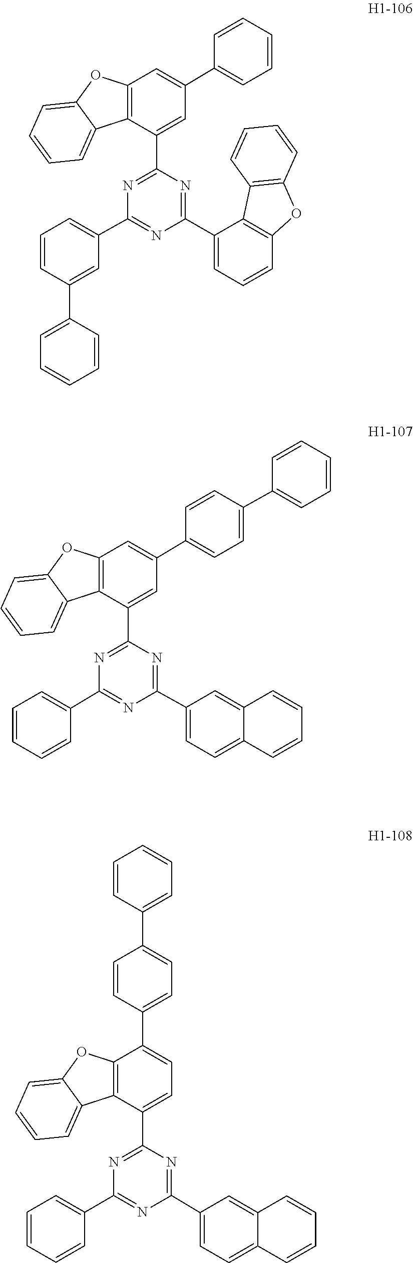 [SMILES: c1ccc(-c2cccc(-c3nc(-c4cccc5oc6ccccc6c45)nc(-c4cc(-c5ccccc5)cc5oc6ccccc6c45)n3)c2)cc1 ; c1ccc(-c2ccc(-c3cc(-c4nc(-c5ccccc5)nc(-c5ccc6ccccc6c5)n4)c4c(c3)oc3ccccc34)cc2)cc1 ; c1ccc(-c2ccc(-c3ccc(-c4nc(-c5ccccc5)nc(-c5ccc6ccccc6c5)n4)c4c3oc3ccccc34)cc2)cc1]

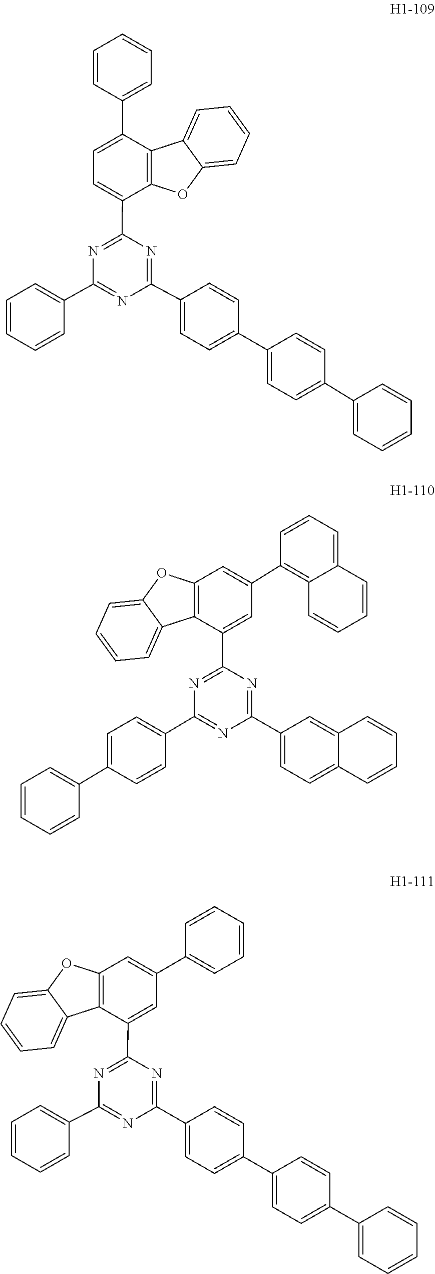 [SMILES: c1ccc(-c2ccc(-c3ccc(-c4nc(-c5ccccc5)nc(-c5ccc(-c6ccccc6)c6c5oc5ccccc56)n4)cc3)cc2)cc1 ; c1ccc(-c2ccc(-c3nc(-c4ccc5ccccc5c4)nc(-c4cc(-c5cccc6ccccc56)cc5oc6ccccc6c45)n3)cc2)cc1 ; c1ccc(-c2ccc(-c3ccc(-c4nc(-c5ccccc5)nc(-c5cc(-c6ccccc6)cc6oc7ccccc7c56)n4)cc3)cc2)cc1]

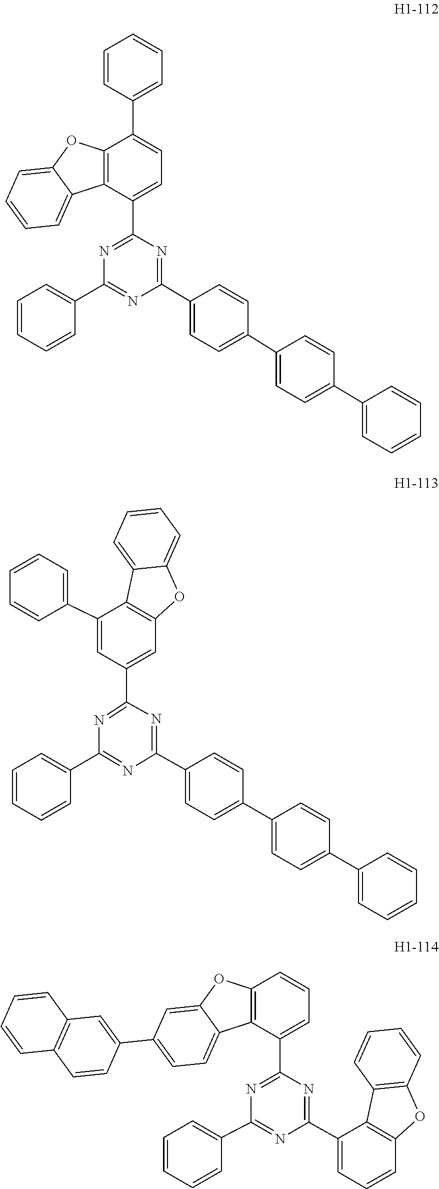

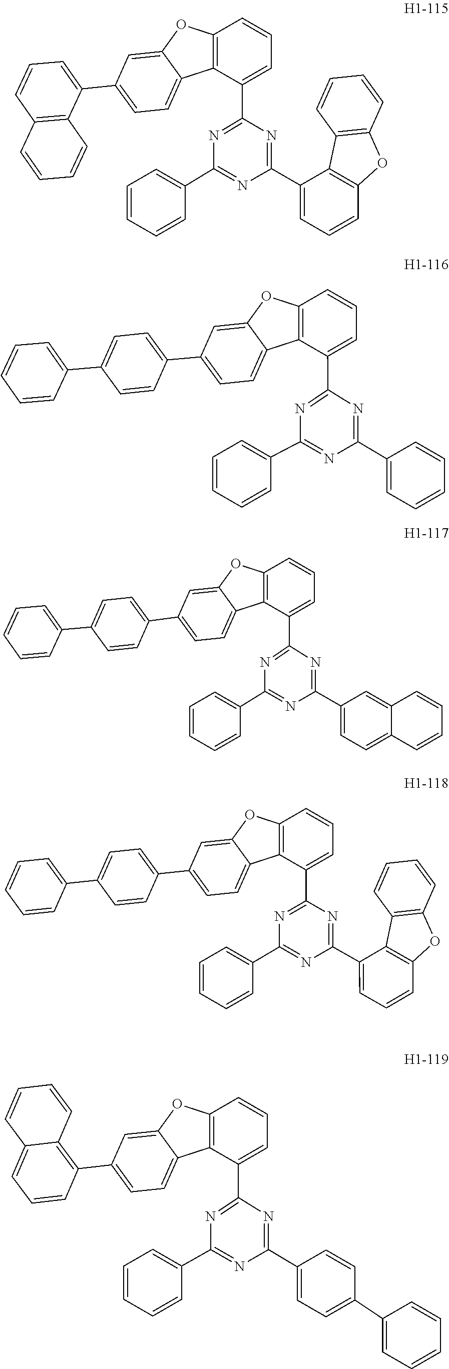

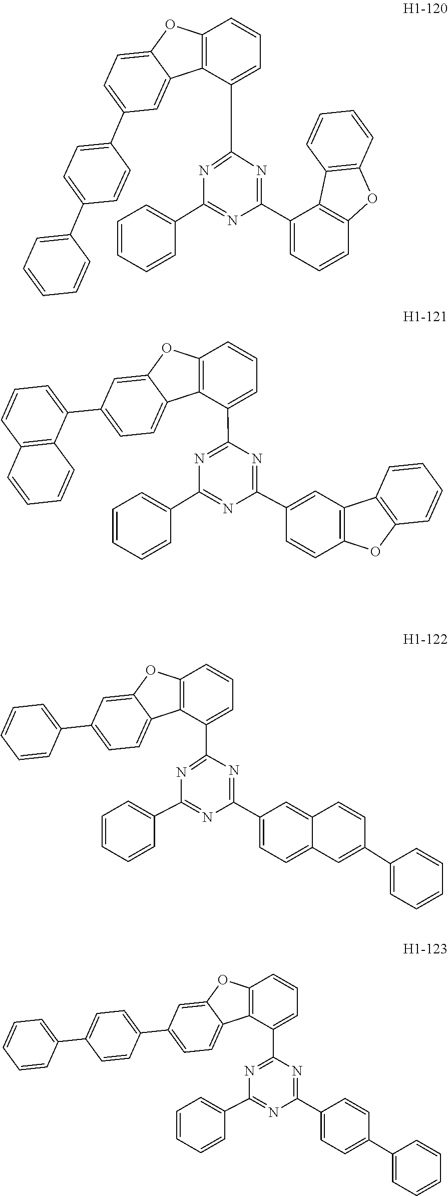

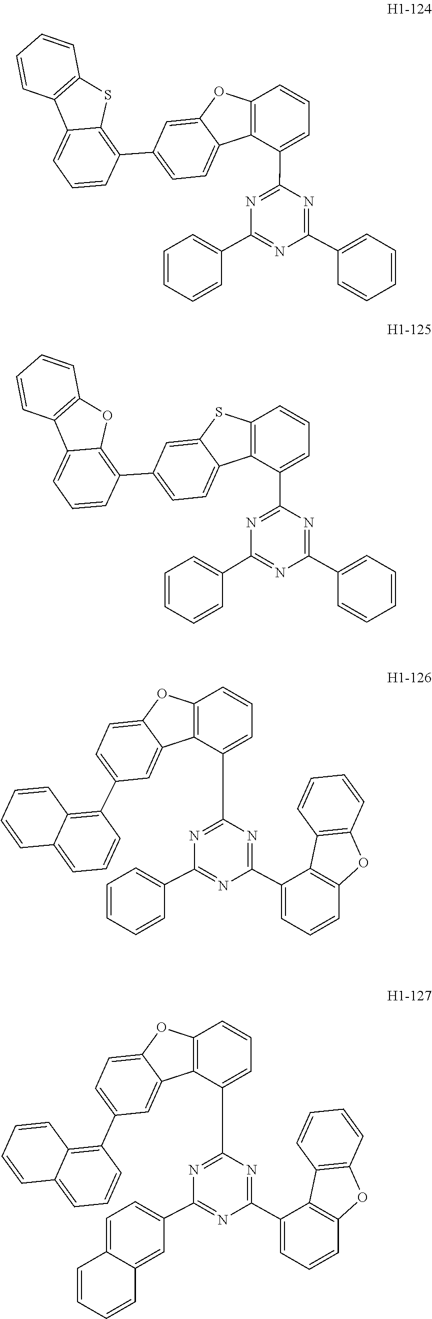 [SMILES: c1ccc(-c2nc(-c3ccccc3)nc(-c3cccc4oc5cc(-c6cccc7c6sc6ccccc67)ccc5c34)n2)cc1 ; c1ccc(-c2nc(-c3ccccc3)nc(-c3cccc4sc5cc(-c6cccc7c6oc6ccccc67)ccc5c34)n2)cc1 ; c1ccc(-c2nc(-c3cccc4oc5ccccc5c34)nc(-c3cccc4oc5ccc(-c6cccc7ccccc67)cc5c34)n2)cc1 ; c1ccc2cc(-c3nc(-c4cccc5oc6ccccc6c45)nc(-c4cccc5oc6ccc(-c7cccc8ccccc78)cc6c45)n3)ccc2c1]

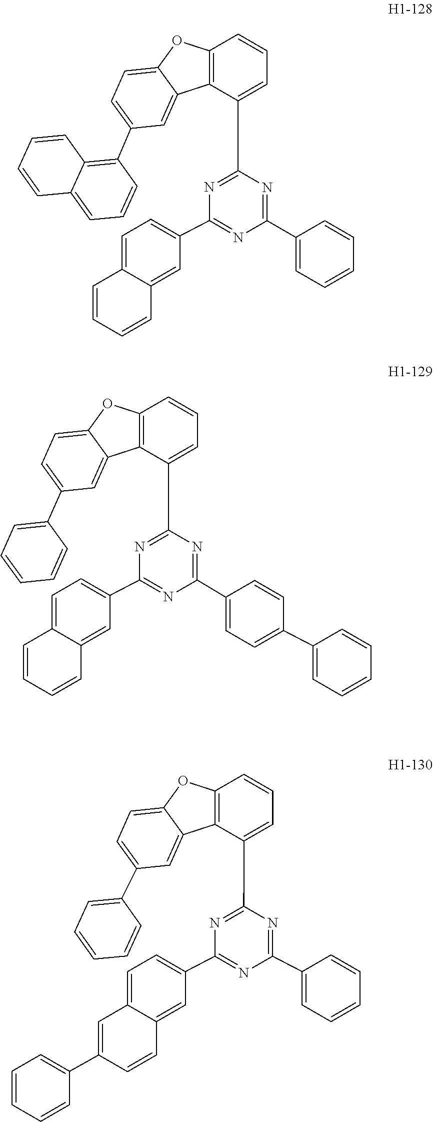 [SMILES: c1ccc(-c2nc(-c3ccc4ccccc4c3)nc(-c3cccc4oc5ccc(-c6cccc7ccccc67)cc5c34)n2)cc1 ; c1ccc(-c2ccc(-c3nc(-c4ccc5ccccc5c4)nc(-c4cccc5oc6ccc(-c7ccccc7)cc6c45)n3)cc2)cc1 ; c1ccc(-c2ccc3cc(-c4nc(-c5ccccc5)nc(-c5cccc6oc7ccc(-c8ccccc8)cc7c56)n4)ccc3c2)cc1]

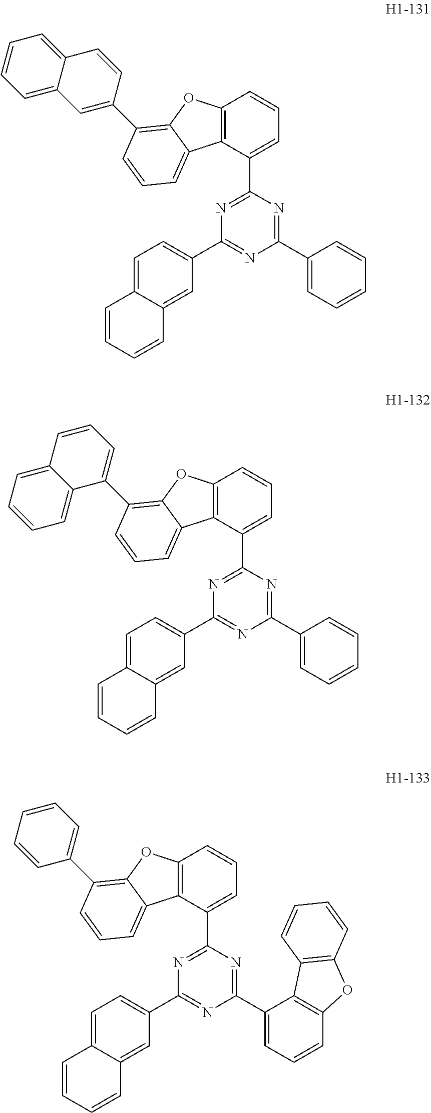 [SMILES: c1ccc(-c2nc(-c3ccc4ccccc4c3)nc(-c3cccc4oc5c(-c6ccc7ccccc7c6)cccc5c34)n2)cc1 ; c1ccc(-c2nc(-c3ccc4ccccc4c3)nc(-c3cccc4oc5c(-c6cccc7ccccc67)cccc5c34)n2)cc1 ; c1ccc(-c2cccc3c2oc2cccc(-c4nc(-c5ccc6ccccc6c5)nc(-c5cccc6oc7ccccc7c56)n4)c23)cc1]

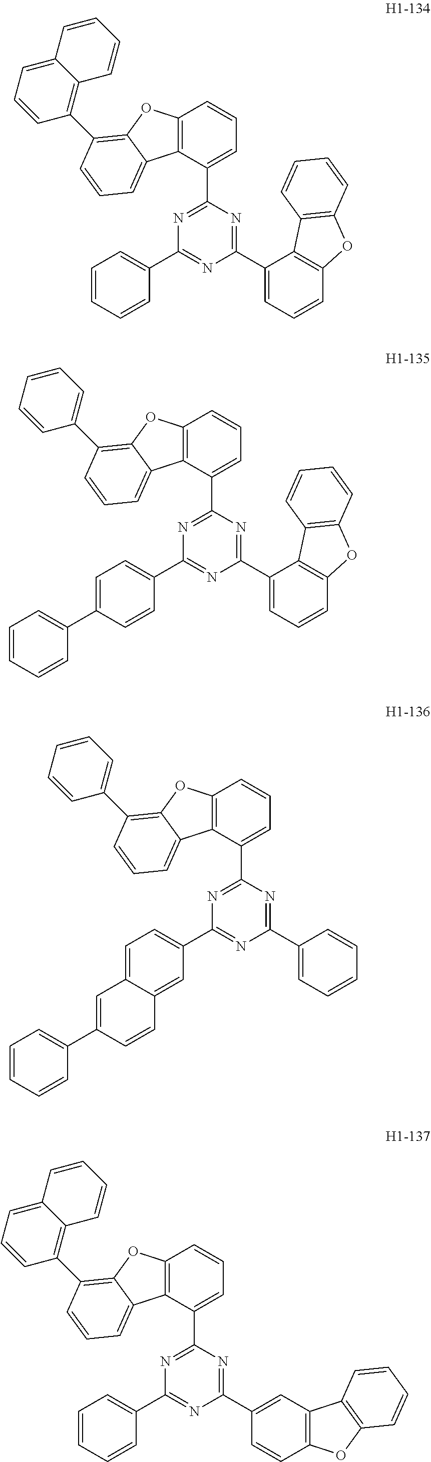 [SMILES: c1ccc(-c2nc(-c3cccc4oc5ccccc5c34)nc(-c3cccc4oc5c(-c6cccc7ccccc67)cccc5c34)n2)cc1 ; c1ccc(-c2ccc(-c3nc(-c4cccc5oc6ccccc6c45)nc(-c4cccc5oc6c(-c7ccccc7)cccc6c45)n3)cc2)cc1 ; c1ccc(-c2ccc3cc(-c4nc(-c5ccccc5)nc(-c5cccc6oc7c(-c8ccccc8)cccc7c56)n4)ccc3c2)cc1 ; c1ccc(-c2nc(-c3ccc4oc5ccccc5c4c3)nc(-c3cccc4oc5c(-c6cccc7ccccc67)cccc5c34)n2)cc1]

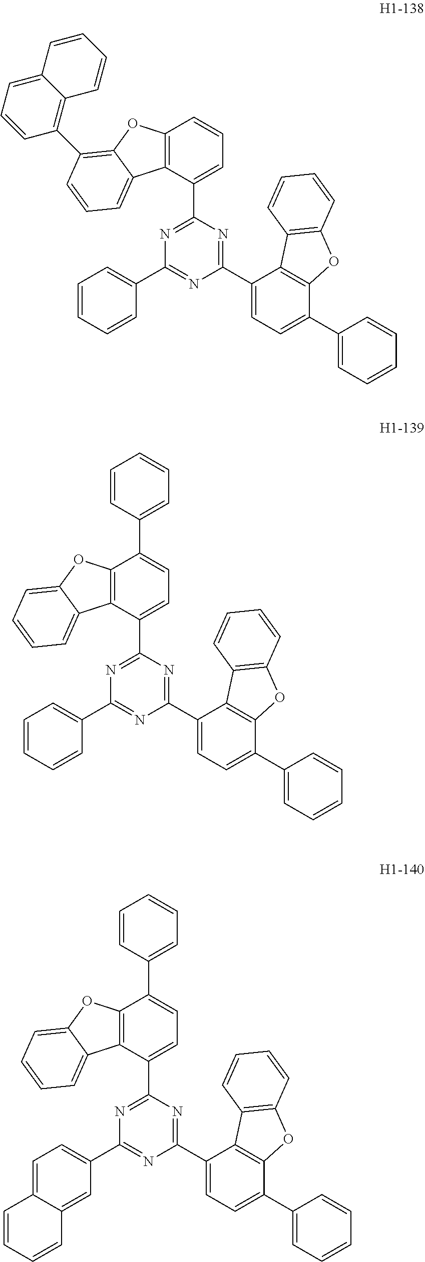 [SMILES: c1ccc(-c2nc(-c3cccc4oc5c(-c6cccc7ccccc67)cccc5c34)nc(-c3ccc(-c4ccccc4)c4oc5ccccc5c34)n2)cc1 ; c1ccc(-c2nc(-c3ccc(-c4ccccc4)c4oc5ccccc5c34)nc(-c3ccc(-c4ccccc4)c4oc5ccccc5c34)n2)cc1 ; c1ccc(-c2ccc(-c3nc(-c4ccc5ccccc5c4)nc(-c4ccc(-c5ccccc5)c5oc6ccccc6c45)n3)c3c2oc2ccccc23)cc1]

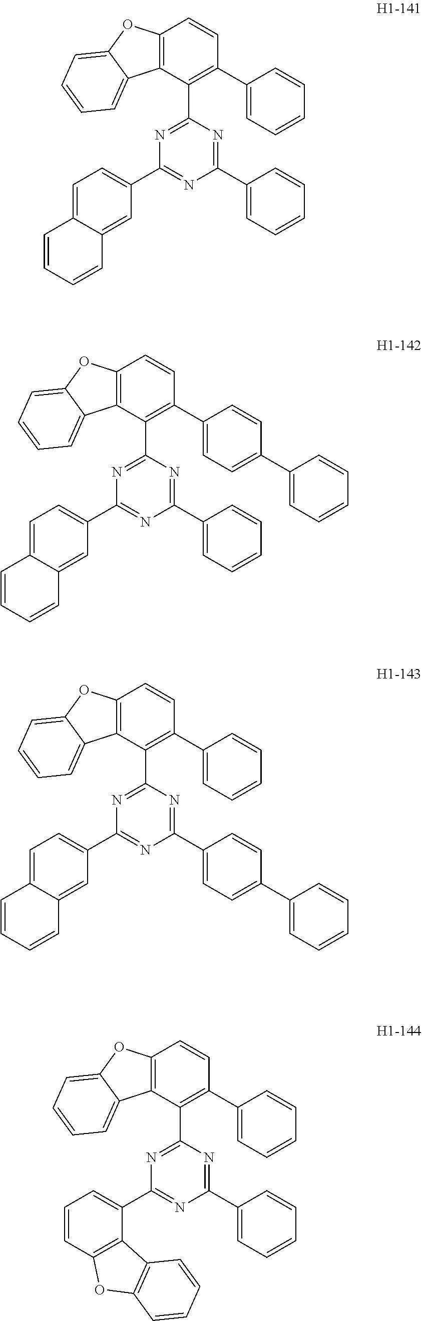 [SMILES: c1ccc(-c2nc(-c3ccc4ccccc4c3)nc(-c3c(-c4ccccc4)ccc4oc5ccccc5c34)n2)cc1 ; c1ccc(-c2ccc(-c3ccc4oc5ccccc5c4c3-c3nc(-c4ccccc4)nc(-c4ccc5ccccc5c4)n3)cc2)cc1 ; c1ccc(-c2ccc(-c3nc(-c4ccc5ccccc5c4)nc(-c4c(-c5ccccc5)ccc5oc6ccccc6c45)n3)cc2)cc1 ; c1ccc(-c2nc(-c3cccc4oc5ccccc5c34)nc(-c3c(-c4ccccc4)ccc4oc5ccccc5c34)n2)cc1]

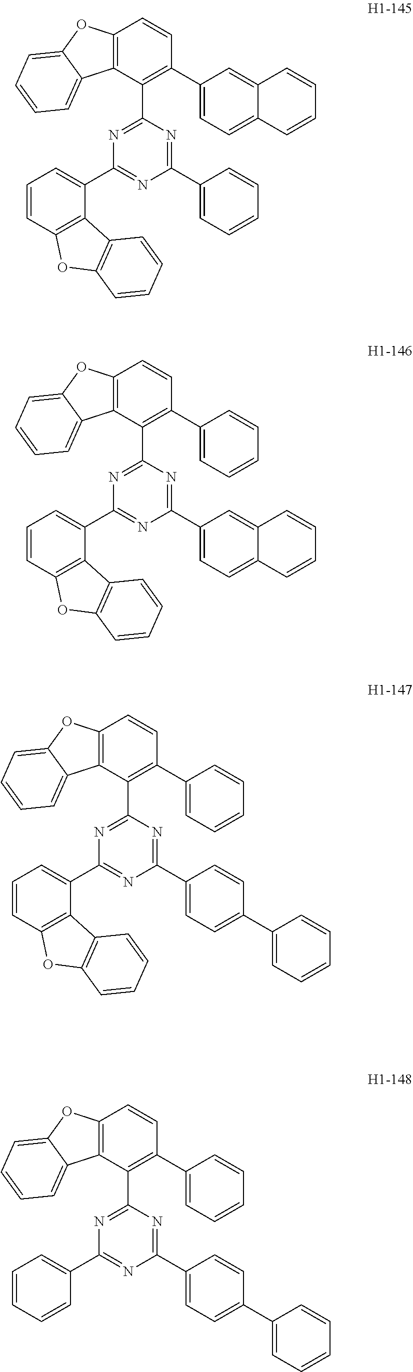 [SMILES: c1ccc(-c2nc(-c3cccc4oc5ccccc5c34)nc(-c3c(-c4ccc5ccccc5c4)ccc4oc5ccccc5c34)n2)cc1 ; c1ccc(-c2ccc3oc4ccccc4c3c2-c2nc(-c3ccc4ccccc4c3)nc(-c3cccc4oc5ccccc5c34)n2)cc1 ; c1ccc(-c2ccc(-c3nc(-c4cccc5oc6ccccc6c45)nc(-c4c(-c5ccccc5)ccc5oc6ccccc6c45)n3)cc2)cc1 ; c1ccc(-c2ccc(-c3nc(-c4ccccc4)nc(-c4c(-c5ccccc5)ccc5oc6ccccc6c45)n3)cc2)cc1]

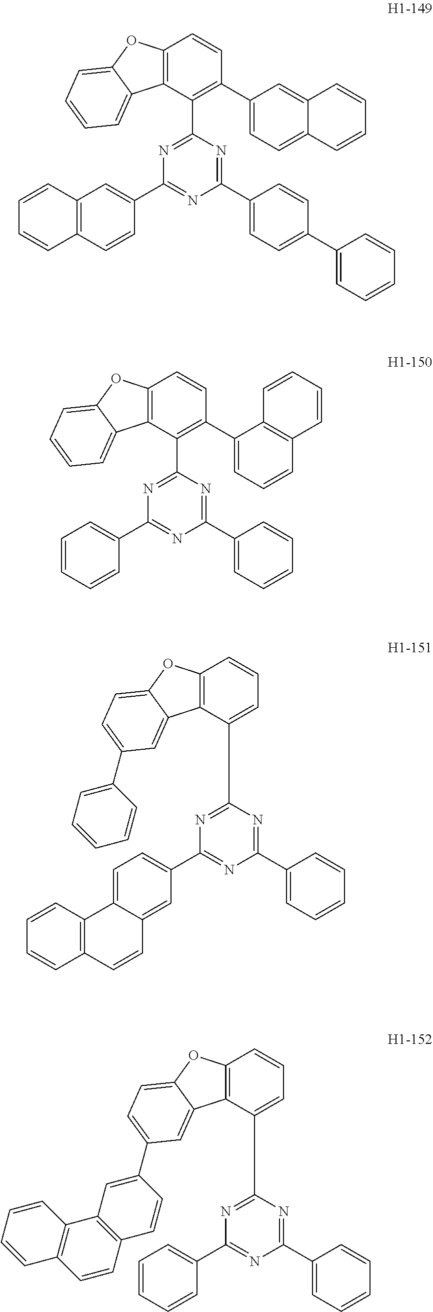 [SMILES: c1ccc(-c2ccc(-c3nc(-c4ccc5ccccc5c4)nc(-c4c(-c5ccc6ccccc6c5)ccc5oc6ccccc6c45)n3)cc2)cc1 ; c1ccc(-c2nc(-c3ccccc3)nc(-c3c(-c4cccc5ccccc45)ccc4oc5ccccc5c34)n2)cc1 ; c1ccc(-c2ccc3oc4cccc(-c5nc(-c6ccccc6)nc(-c6ccc7c(ccc8ccccc87)c6)n5)c4c3c2)cc1 ; c1ccc(-c2nc(-c3ccccc3)nc(-c3cccc4oc5ccc(-c6ccc7ccc8ccccc8c7c6)cc5c34)n2)cc1]

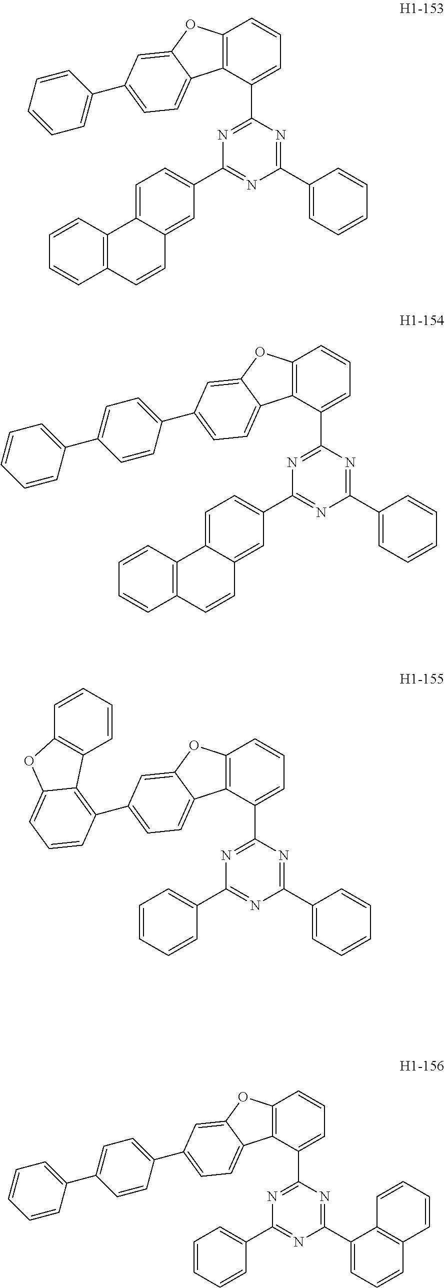 [SMILES: c1ccc(-c2ccc3c(c2)oc2cccc(-c4nc(-c5ccccc5)nc(-c5ccc6c(ccc7ccccc76)c5)n4)c23)cc1 ; c1ccc(-c2ccc(-c3ccc4c(c3)oc3cccc(-c5nc(-c6ccccc6)nc(-c6ccc7c(ccc8ccccc87)c6)n5)c34)cc2)cc1 ; c1ccc(-c2nc(-c3ccccc3)nc(-c3cccc4oc5cc(-c6cccc7oc8ccccc8c67)ccc5c34)n2)cc1 ; c1ccc(-c2ccc(-c3ccc4c(c3)oc3cccc(-c5nc(-c6ccccc6)nc(-c6cccc7ccccc67)n5)c34)cc2)cc1]

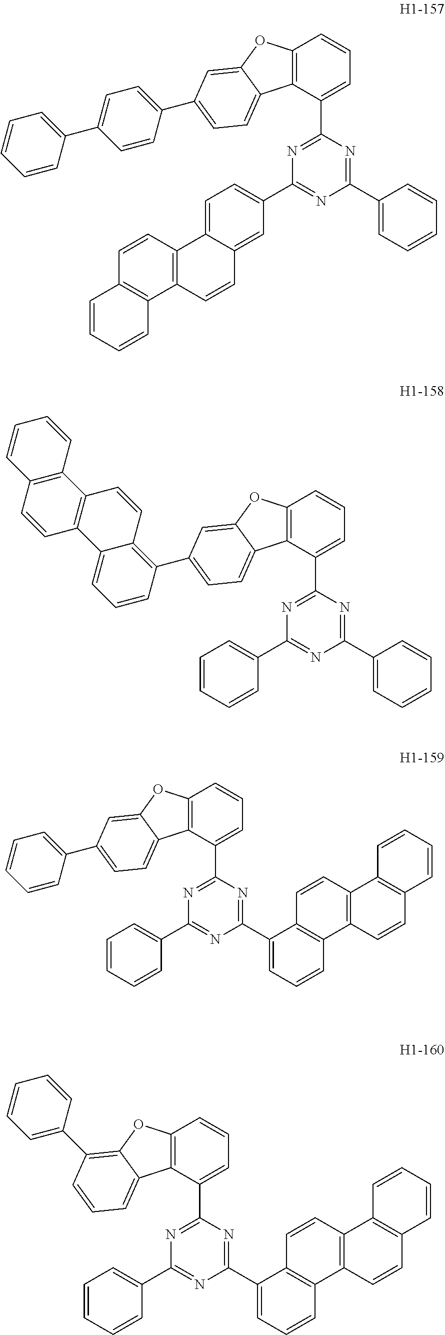 [SMILES: c1ccc(-c2ccc(-c3ccc4c(c3)oc3cccc(-c5nc(-c6ccccc6)nc(-c6ccc7c(ccc8c9ccccc9ccc78)c6)n5)c34)cc2)cc1 ; c1ccc(-c2nc(-c3ccccc3)nc(-c3cccc4oc5cc(-c6cccc7c6ccc6c8ccccc8ccc76)ccc5c34)n2)cc1 ; c1ccc(-c2ccc3c(c2)oc2cccc(-c4nc(-c5ccccc5)nc(-c5cccc6c5ccc5c7ccccc7ccc65)n4)c23)cc1 ; c1ccc(-c2nc(-c3cccc4c3ccc3c5ccccc5ccc43)nc(-c3cccc4oc5c(-c6ccccc6)cccc5c34)n2)cc1]

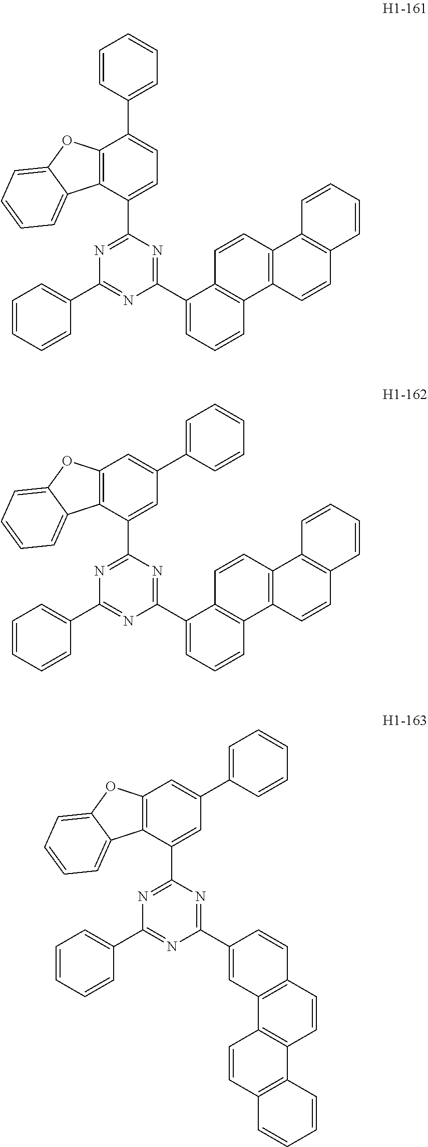 [SMILES: c1ccc(-c2nc(-c3cccc4c3ccc3c5ccccc5ccc43)nc(-c3ccc(-c4ccccc4)c4oc5ccccc5c34)n2)cc1 ; c1ccc(-c2cc(-c3nc(-c4ccccc4)nc(-c4cccc5c4ccc4c6ccccc6ccc54)n3)c3c(c2)oc2ccccc23)cc1 ; c1ccc(-c2cc(-c3nc(-c4ccccc4)nc(-c4ccc5ccc6c7ccccc7ccc6c5c4)n3)c3c(c2)oc2ccccc23)cc1]

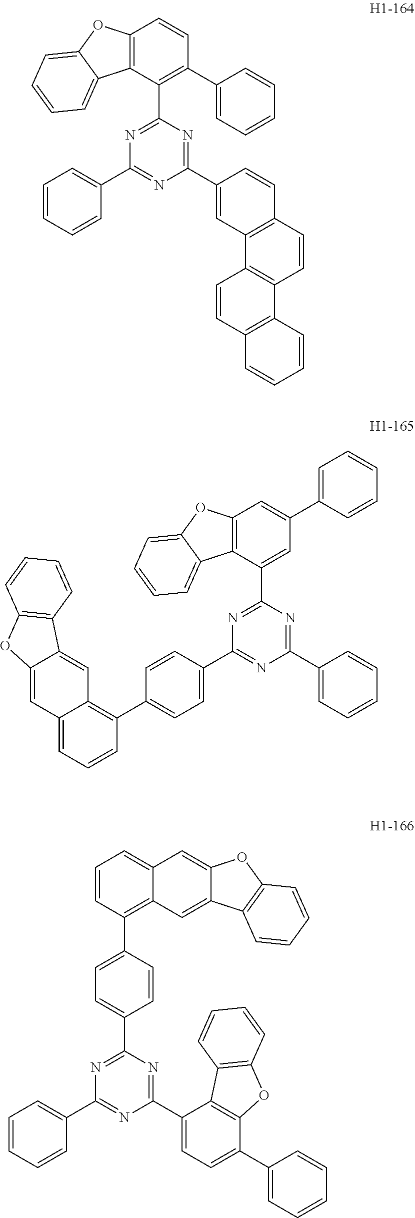 [SMILES: c1ccc(-c2nc(-c3ccc4ccc5c6ccccc6ccc5c4c3)nc(-c3c(-c4ccccc4)ccc4oc5ccccc5c34)n2)cc1 ; c1ccc(-c2cc(-c3nc(-c4ccccc4)nc(-c4ccc(-c5cccc6cc7oc8ccccc8c7cc56)cc4)n3)c3c(c2)oc2ccccc23)cc1 ; c1ccc(-c2nc(-c3ccc(-c4cccc5cc6oc7ccccc7c6cc45)cc3)nc(-c3ccc(-c4ccccc4)c4oc5ccccc5c34)n2)cc1]

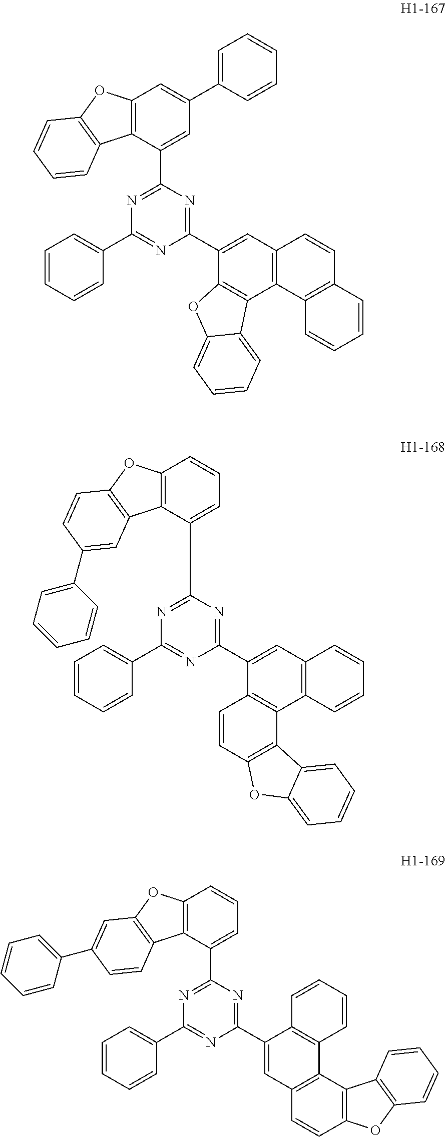 [SMILES: c1ccc(-c2cc(-c3nc(-c4ccccc4)nc(-c4cc5ccc6ccccc6c5c5c4oc4ccccc45)n3)c3c(c2)oc2ccccc23)cc1 ; c1ccc(-c2ccc3oc4cccc(-c5nc(-c6ccccc6)nc(-c6cc7ccccc7c7c6ccc6oc8ccccc8c67)n5)c4c3c2)cc1 ; c1ccc(-c2ccc3c(c2)oc2cccc(-c4nc(-c5ccccc5)nc(-c5cc6ccc7oc8ccccc8c7c6c6ccccc56)n4)c23)cc1]

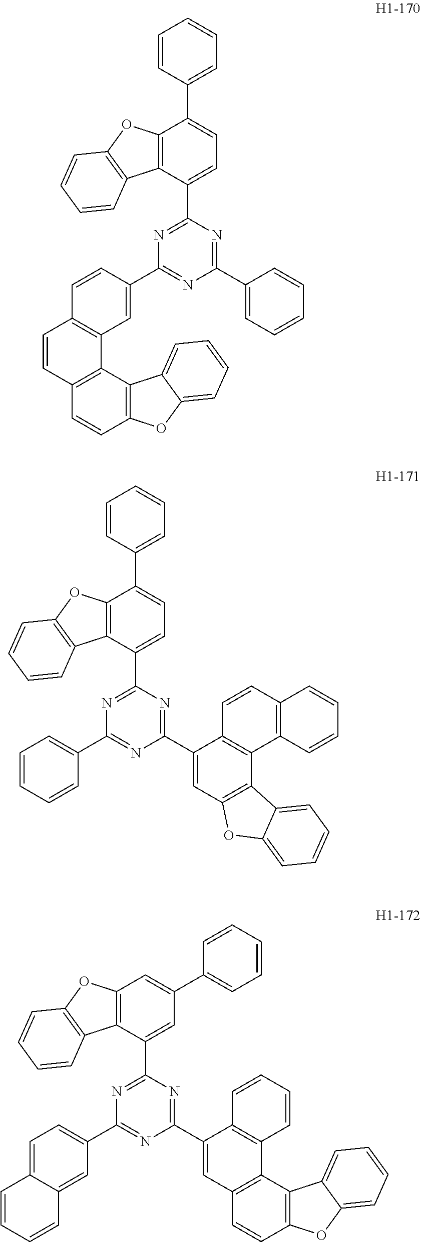 [SMILES: c1ccc(-c2nc(-c3ccc4ccc5ccc6oc7ccccc7c6c5c4c3)nc(-c3ccc(-c4ccccc4)c4oc5ccccc5c34)n2)cc1 ; c1ccc(-c2nc(-c3cc4oc5ccccc5c4c4c3ccc3ccccc34)nc(-c3ccc(-c4ccccc4)c4oc5ccccc5c34)n2)cc1 ; c1ccc(-c2cc(-c3nc(-c4ccc5ccccc5c4)nc(-c4cc5ccc6oc7ccccc7c6c5c5ccccc45)n3)c3c(c2)oc2ccccc23)cc1]

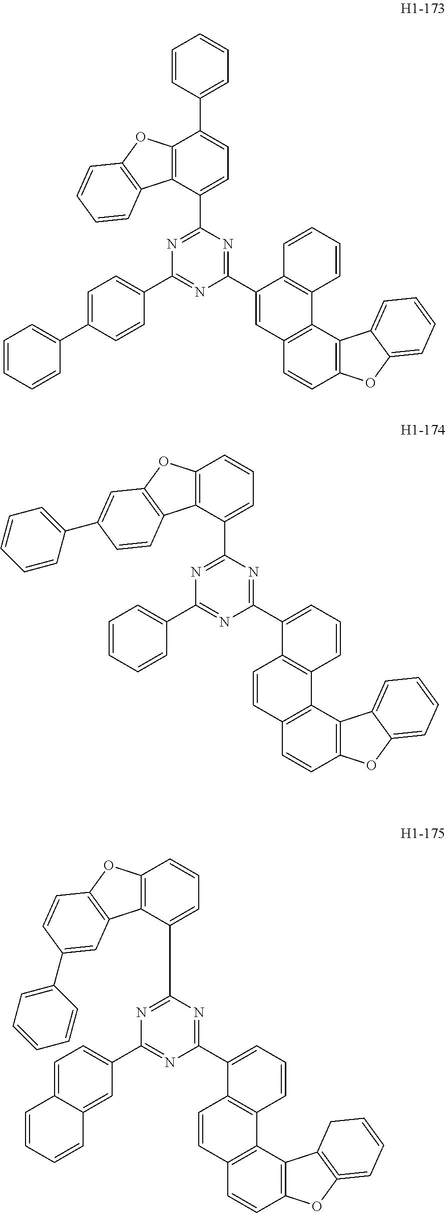

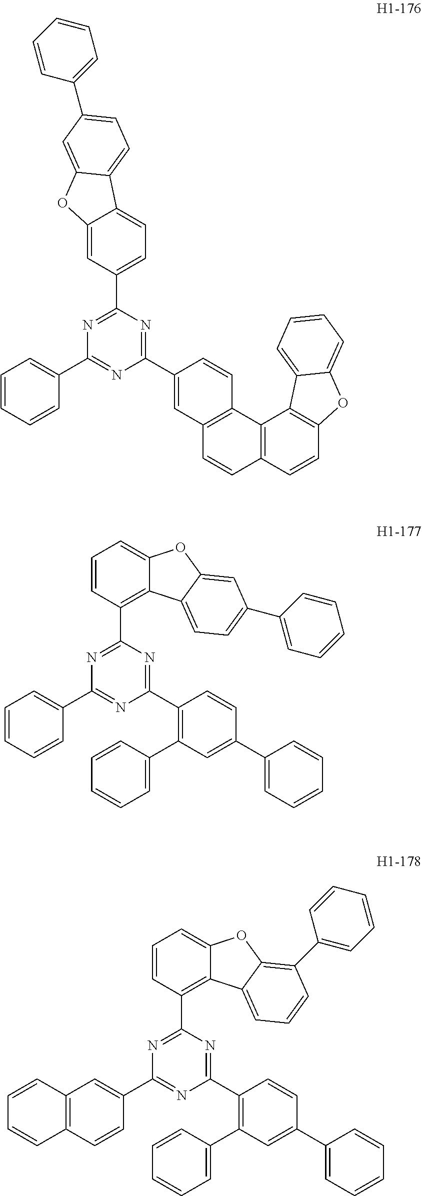 [SMILES: c1ccc(-c2ccc3c(c2)oc2cc(-c4nc(-c5ccccc5)nc(-c5ccc6c(ccc7ccc8oc9ccccc9c8c76)c5)n4)ccc23)cc1 ; c1ccc(-c2ccc(-c3nc(-c4ccccc4)nc(-c4cccc5oc6cc(-c7ccccc7)ccc6c45)n3)c(-c3ccccc3)c2)cc1 ; c1ccc(-c2ccc(-c3nc(-c4ccc5ccccc5c4)nc(-c4cccc5oc6c(-c7ccccc7)cccc6c45)n3)c(-c3ccccc3)c2)cc1]

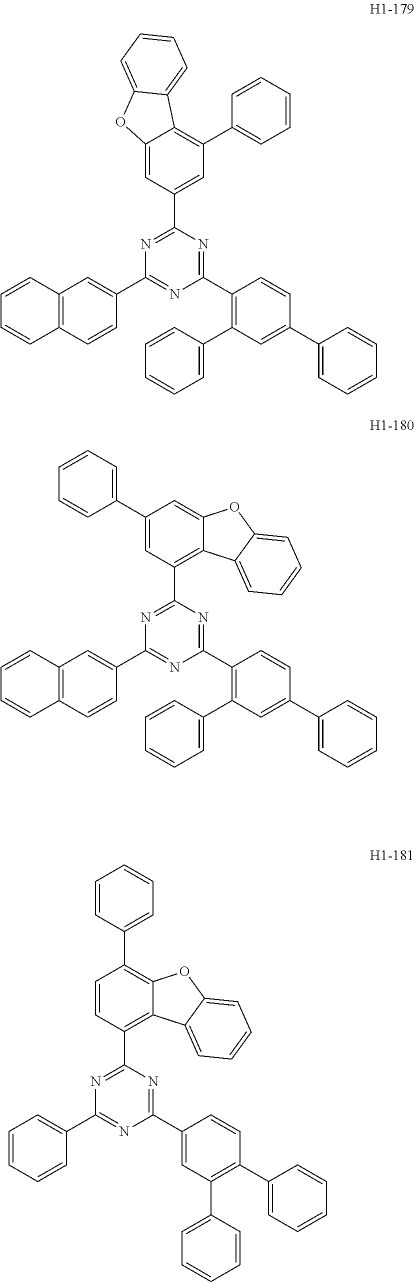 [SMILES: c1ccc(-c2ccc(-c3nc(-c4ccc5ccccc5c4)nc(-c4cc(-c5ccccc5)c5c(c4)oc4ccccc45)n3)c(-c3ccccc3)c2)cc1 ; c1ccc(-c2ccc(-c3nc(-c4ccc5ccccc5c4)nc(-c4cc(-c5ccccc5)cc5oc6ccccc6c45)n3)c(-c3ccccc3)c2)cc1 ; c1ccc(-c2nc(-c3ccc(-c4ccccc4)c(-c4ccccc4)c3)nc(-c3ccc(-c4ccccc4)c4oc5ccccc5c34)n2)cc1]

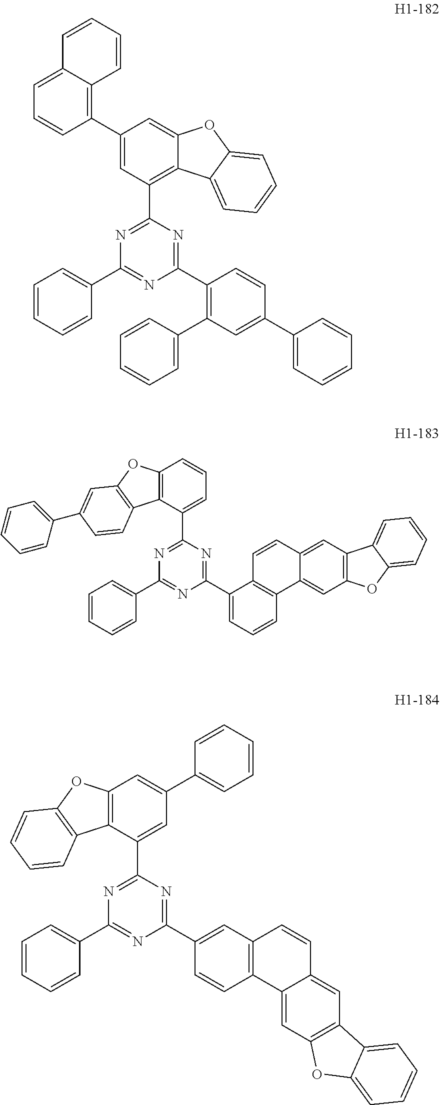 [SMILES: c1ccc(-c2ccc(-c3nc(-c4ccccc4)nc(-c4cc(-c5cccc6ccccc56)cc5oc6ccccc6c45)n3)c(-c3ccccc3)c2)cc1 ; c1ccc(-c2ccc3c(c2)oc2cccc(-c4nc(-c5ccccc5)nc(-c5cccc6c5ccc5cc7c(cc56)oc5ccccc57)n4)c23)cc1 ; c1ccc(-c2cc(-c3nc(-c4ccccc4)nc(-c4ccc5c(ccc6cc7c(cc65)oc5ccccc57)c4)n3)c3c(c2)oc2ccccc23)cc1]

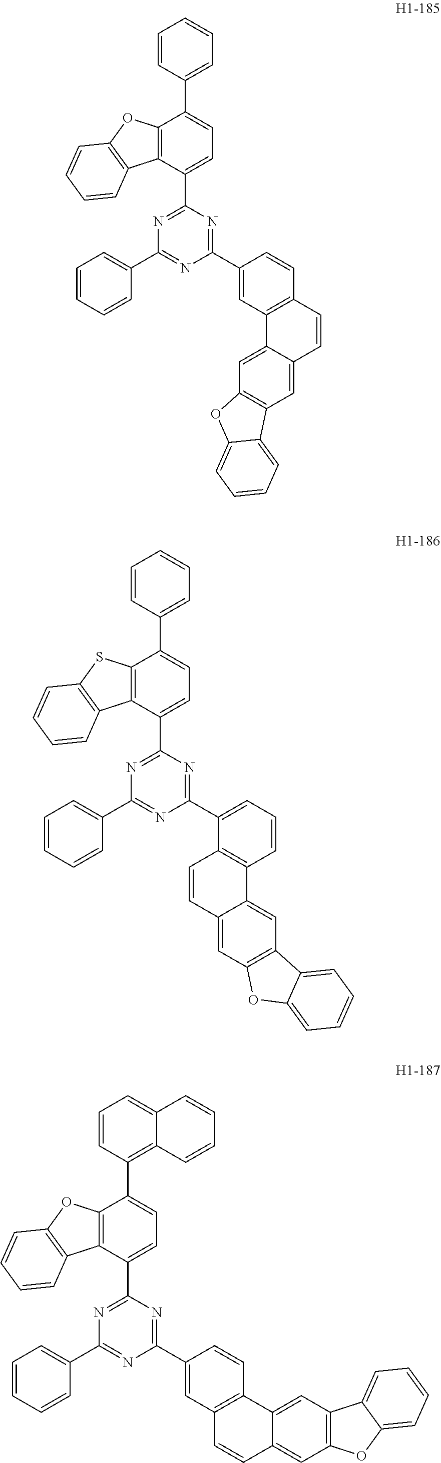 [SMILES: c1ccc(-c2nc(-c3ccc4ccc5cc6c(cc5c4c3)oc3ccccc36)nc(-c3ccc(-c4ccccc4)c4oc5ccccc5c34)n2)cc1 ; c1ccc(-c2nc(-c3cccc4c3ccc3cc5oc6ccccc6c5cc34)nc(-c3ccc(-c4ccccc4)c4sc5ccccc5c34)n2)cc1 ; c1ccc(-c2nc(-c3ccc4c(ccc5cc6oc7ccccc7c6cc54)c3)nc(-c3ccc(-c4cccc5ccccc45)c4oc5ccccc5c34)n2)cc1]

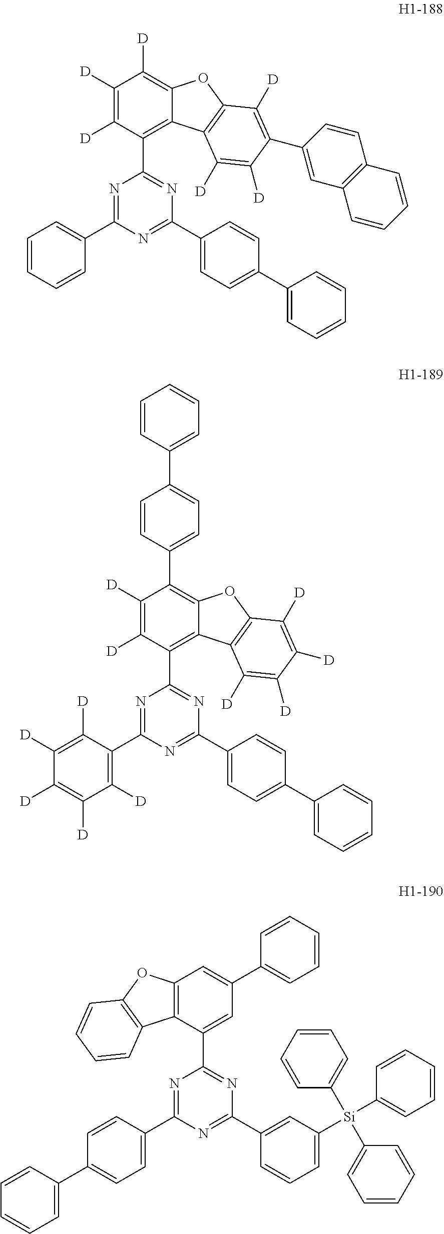 [SMILES: [2H]c1c([2H])c(-c2nc(-c3ccccc3)nc(-c3ccc(-c4ccccc4)cc3)n2)c2c(oc3c([2H])c(-c4ccc5ccccc5c4)c([2H])c([2H])c32)c1[2H] ; [2H]c1c([2H])c([2H])c(-c2nc(-c3ccc(-c4ccccc4)cc3)nc(-c3c([2H])c([2H])c(-c4ccc(-c5ccccc5)cc4)c4oc5c([2H])c([2H])c([2H])c([2H])c5c34)n2)c([2H])c1[2H] ; c1ccc(-c2ccc(-c3nc(-c4cccc([Si](c5ccccc5)(c5ccccc5)c5ccccc5)c4)nc(-c4cc(-c5ccccc5)cc5oc6ccccc6c45)n3)cc2)cc1]

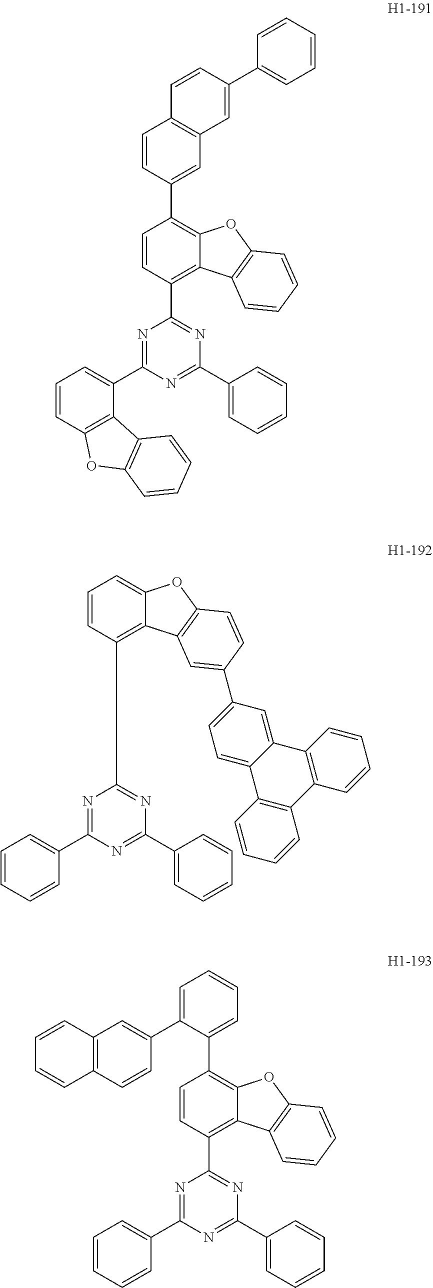 [SMILES: c1ccc(-c2ccc3ccc(-c4ccc(-c5nc(-c6ccccc6)nc(-c6cccc7oc8ccccc8c67)n5)c5c4oc4ccccc45)cc3c2)cc1 ; c1ccc(-c2nc(-c3ccccc3)nc(-c3cccc4oc5ccc(-c6ccc7c8ccccc8c8ccccc8c7c6)cc5c34)n2)cc1 ; c1ccc(-c2nc(-c3ccccc3)nc(-c3ccc(-c4ccccc4-c4ccc5ccccc5c4)c4oc5ccccc5c34)n2)cc1]

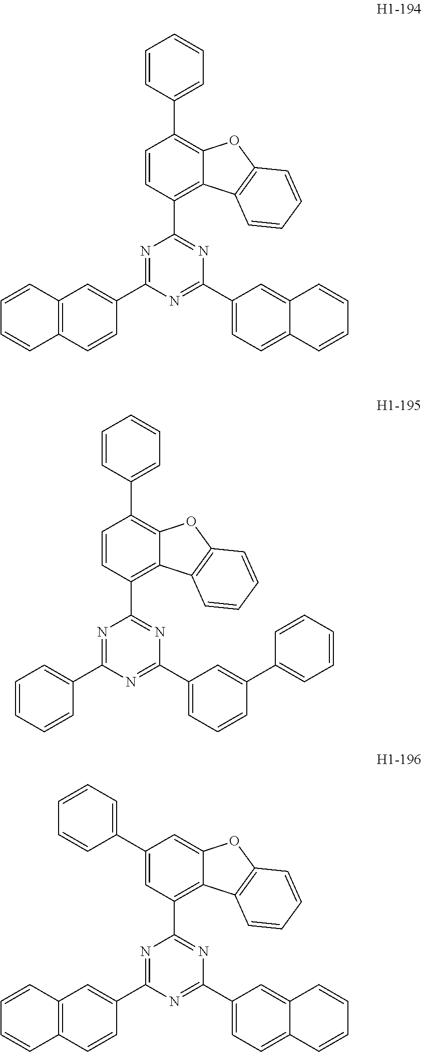 [SMILES: c1ccc(-c2ccc(-c3nc(-c4ccc5ccccc5c4)nc(-c4ccc5ccccc5c4)n3)c3c2oc2ccccc23)cc1 ; c1ccc(-c2cccc(-c3nc(-c4ccccc4)nc(-c4ccc(-c5ccccc5)c5oc6ccccc6c45)n3)c2)cc1 ; c1ccc(-c2cc(-c3nc(-c4ccc5ccccc5c4)nc(-c4ccc5ccccc5c4)n3)c3c(c2)oc2ccccc23)cc1]

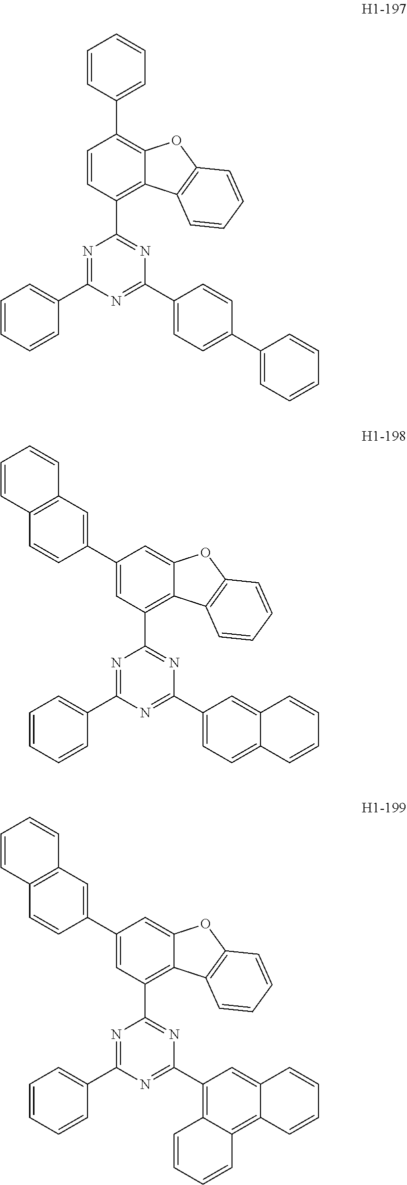 [SMILES: c1ccc(-c2ccc(-c3nc(-c4ccccc4)nc(-c4ccc(-c5ccccc5)c5oc6ccccc6c45)n3)cc2)cc1 ; c1ccc(-c2nc(-c3ccc4ccccc4c3)nc(-c3cc(-c4ccc5ccccc5c4)cc4oc5ccccc5c34)n2)cc1 ; c1ccc(-c2nc(-c3cc4ccccc4c4ccccc34)nc(-c3cc(-c4ccc5ccccc5c4)cc4oc5ccccc5c34)n2)cc1]

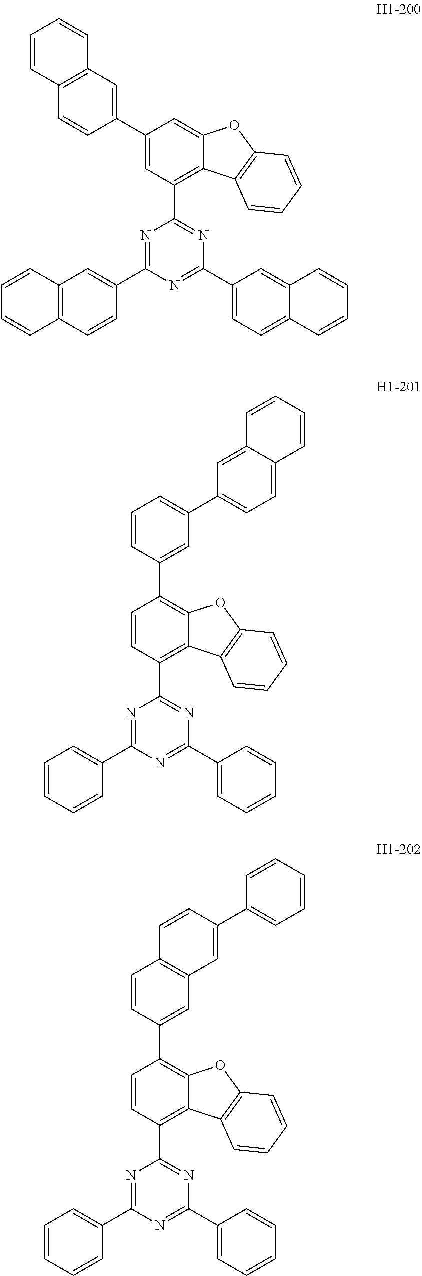 [SMILES: c1ccc2cc(-c3cc(-c4nc(-c5ccc6ccccc6c5)nc(-c5ccc6ccccc6c5)n4)c4c(c3)oc3ccccc34)ccc2c1 ; c1ccc(-c2nc(-c3ccccc3)nc(-c3ccc(-c4cccc(-c5ccc6ccccc6c5)c4)c4oc5ccccc5c34)n2)cc1 ; c1ccc(-c2ccc3ccc(-c4ccc(-c5nc(-c6ccccc6)nc(-c6ccccc6)n5)c5c4oc4ccccc45)cc3c2)cc1]

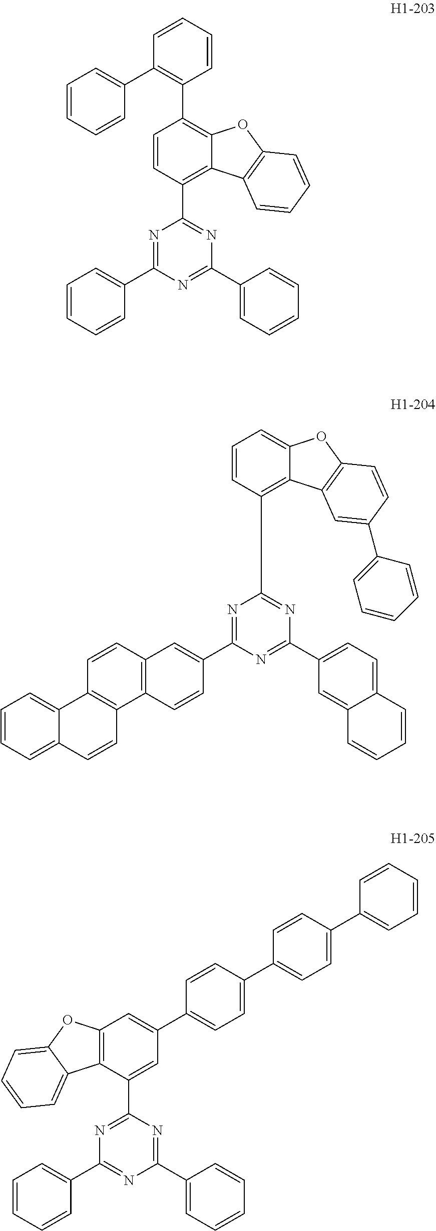 [SMILES: c1ccc(-c2nc(-c3ccccc3)nc(-c3ccc(-c4ccccc4-c4ccccc4)c4oc5ccccc5c34)n2)cc1 ; c1ccc(-c2ccc3oc4cccc(-c5nc(-c6ccc7ccccc7c6)nc(-c6ccc7c(ccc8c9ccccc9ccc78)c6)n5)c4c3c2)cc1 ; c1ccc(-c2ccc(-c3ccc(-c4cc(-c5nc(-c6ccccc6)nc(-c6ccccc6)n5)c5c(c4)oc4ccccc45)cc3)cc2)cc1]

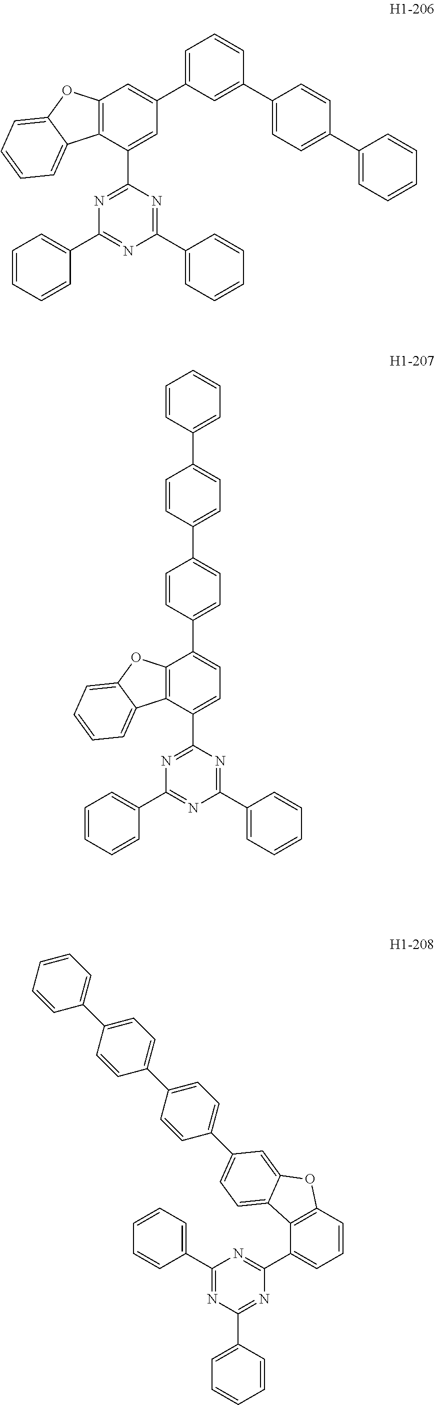 [SMILES: c1ccc(-c2ccc(-c3cccc(-c4cc(-c5nc(-c6ccccc6)nc(-c6ccccc6)n5)c5c(c4)oc4ccccc45)c3)cc2)cc1 ; c1ccc(-c2ccc(-c3ccc(-c4ccc(-c5nc(-c6ccccc6)nc(-c6ccccc6)n5)c5c4oc4ccccc45)cc3)cc2)cc1 ; c1ccc(-c2ccc(-c3ccc(-c4ccc5c(c4)oc4cccc(-c6nc(-c7ccccc7)nc(-c7ccccc7)n6)c45)cc3)cc2)cc1]

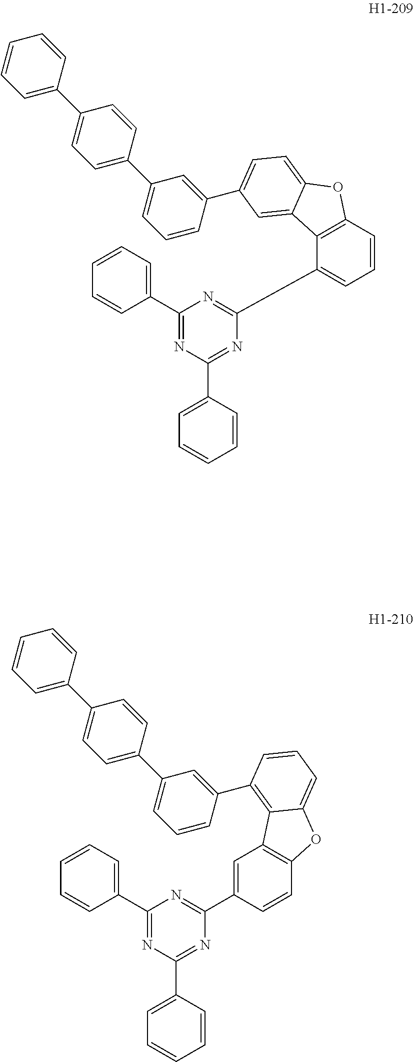 [SMILES: c1ccc(-c2ccc(-c3cccc(-c4ccc5oc6cccc(-c7nc(-c8ccccc8)nc(-c8ccccc8)n7)c6c5c4)c3)cc2)cc1 ; c1ccc(-c2ccc(-c3cccc(-c4cccc5oc6ccc(-c7nc(-c8ccccc8)nc(-c8ccccc8)n7)cc6c45)c3)cc2)cc1]

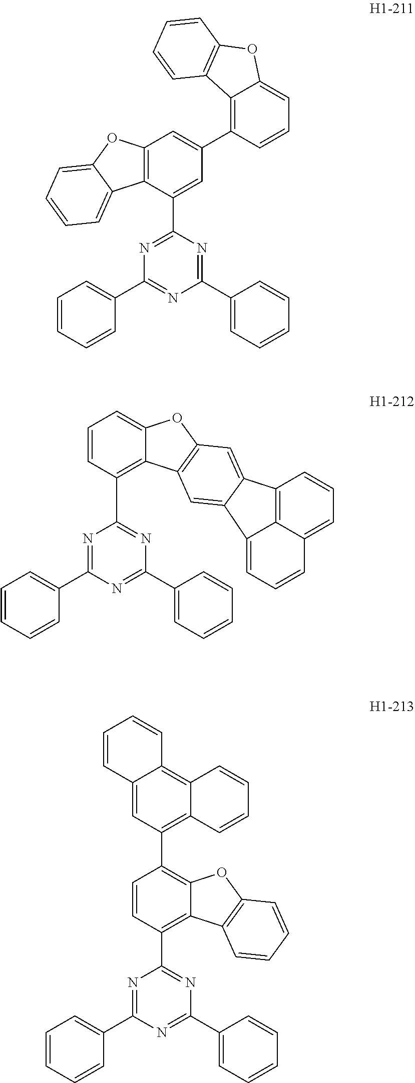 [SMILES: c1ccc(-c2nc(-c3ccccc3)nc(-c3cc(-c4cccc5oc6ccccc6c45)cc4oc5ccccc5c34)n2)cc1 ; c1ccc(-c2nc(-c3ccccc3)nc(-c3cccc4oc5cc6c(cc5c34)-c3cccc4cccc-6c34)n2)cc1 ; c1ccc(-c2nc(-c3ccccc3)nc(-c3ccc(-c4cc5ccccc5c5ccccc45)c4oc5ccccc5c34)n2)cc1]

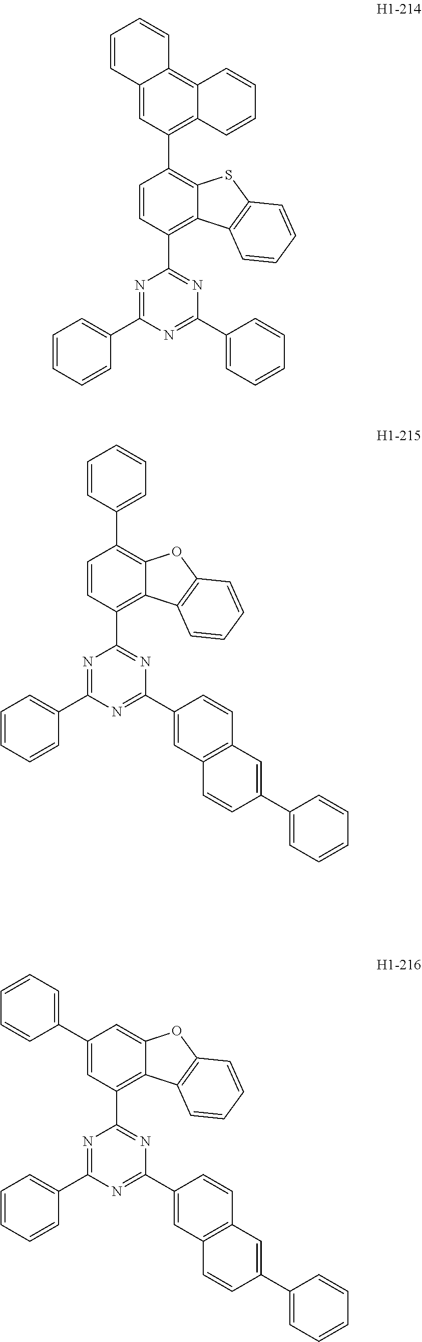 [SMILES: c1ccc(-c2nc(-c3ccccc3)nc(-c3ccc(-c4cc5ccccc5c5ccccc45)c4sc5ccccc5c34)n2)cc1 ; c1ccc(-c2ccc3cc(-c4nc(-c5ccccc5)nc(-c5ccc(-c6ccccc6)c6oc7ccccc7c56)n4)ccc3c2)cc1 ; c1ccc(-c2ccc3cc(-c4nc(-c5ccccc5)nc(-c5cc(-c6ccccc6)cc6oc7ccccc7c56)n4)ccc3c2)cc1]

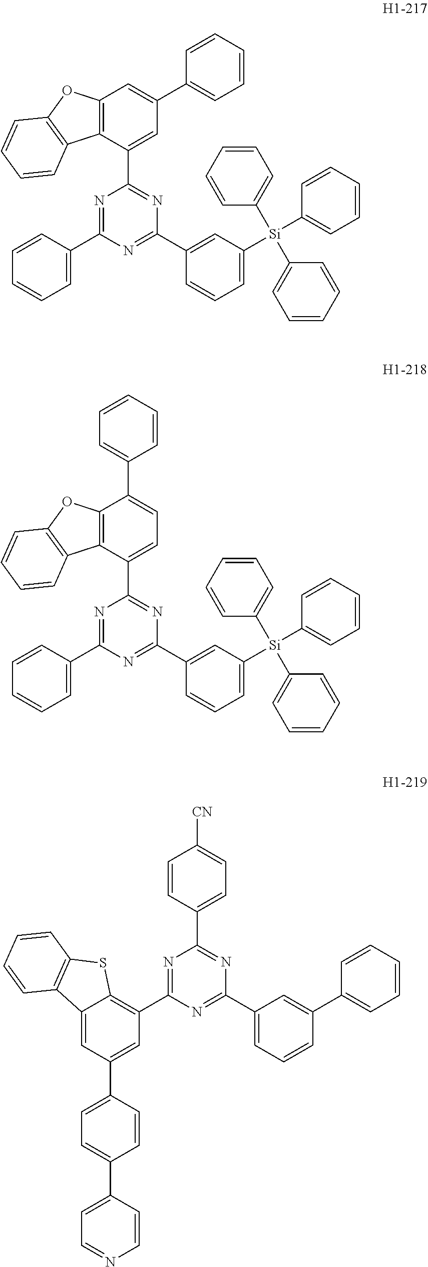 [SMILES: c1ccc(-c2cc(-c3nc(-c4ccccc4)nc(-c4cccc([Si](c5ccccc5)(c5ccccc5)c5ccccc5)c4)n3)c3c(c2)oc2ccccc23)cc1 ; c1ccc(-c2nc(-c3cccc([Si](c4ccccc4)(c4ccccc4)c4ccccc4)c3)nc(-c3ccc(-c4ccccc4)c4oc5ccccc5c34)n2)cc1 ; N#Cc1ccc(-c2nc(-c3cccc(-c4ccccc4)c3)nc(-c3cc(-c4ccc(-c5ccncc5)cc4)cc4c3sc3ccccc34)n2)cc1]

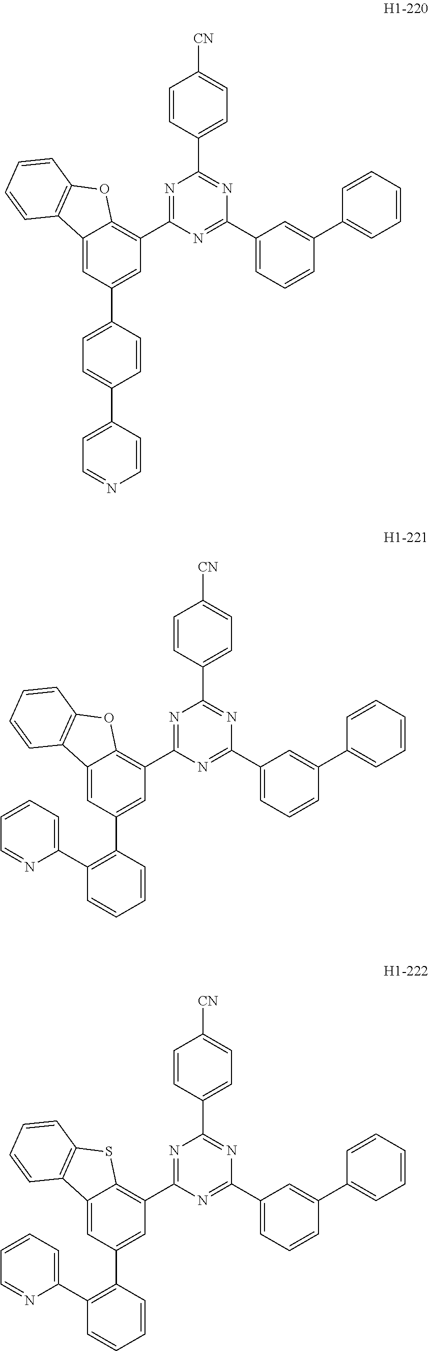 [SMILES: N#Cc1ccc(-c2nc(-c3cccc(-c4ccccc4)c3)nc(-c3cc(-c4ccc(-c5ccncc5)cc4)cc4c3oc3ccccc34)n2)cc1 ; N#Cc1ccc(-c2nc(-c3cccc(-c4ccccc4)c3)nc(-c3cc(-c4ccccc4-c4ccccn4)cc4c3oc3ccccc34)n2)cc1 ; N#Cc1ccc(-c2nc(-c3cccc(-c4ccccc4)c3)nc(-c3cc(-c4ccccc4-c4ccccn4)cc4c3sc3ccccc34)n2)cc1]

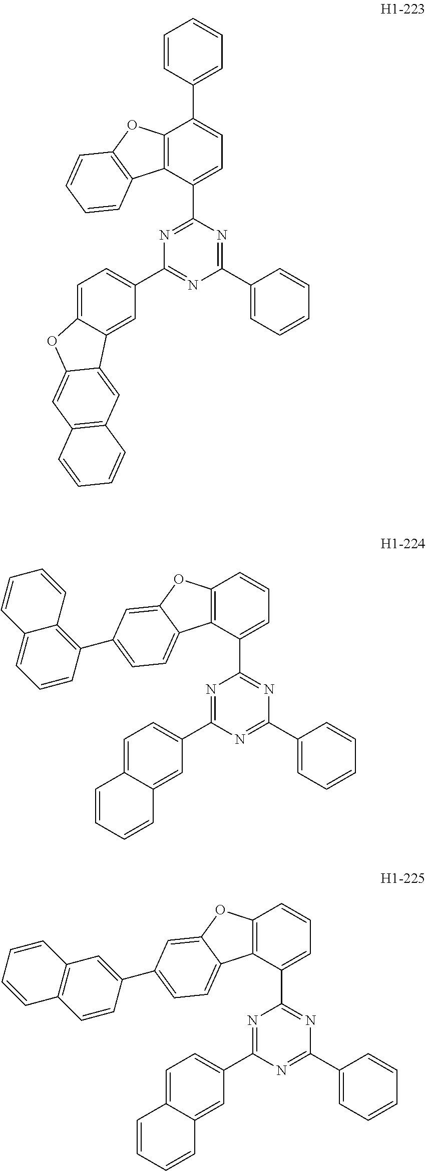 [SMILES: c1ccc(-c2nc(-c3ccc4oc5cc6ccccc6cc5c4c3)nc(-c3ccc(-c4ccccc4)c4oc5ccccc5c34)n2)cc1 ; c1ccc(-c2nc(-c3ccc4ccccc4c3)nc(-c3cccc4oc5cc(-c6cccc7ccccc67)ccc5c34)n2)cc1 ; c1ccc(-c2nc(-c3ccc4ccccc4c3)nc(-c3cccc4oc5cc(-c6ccc7ccccc7c6)ccc5c34)n2)cc1]

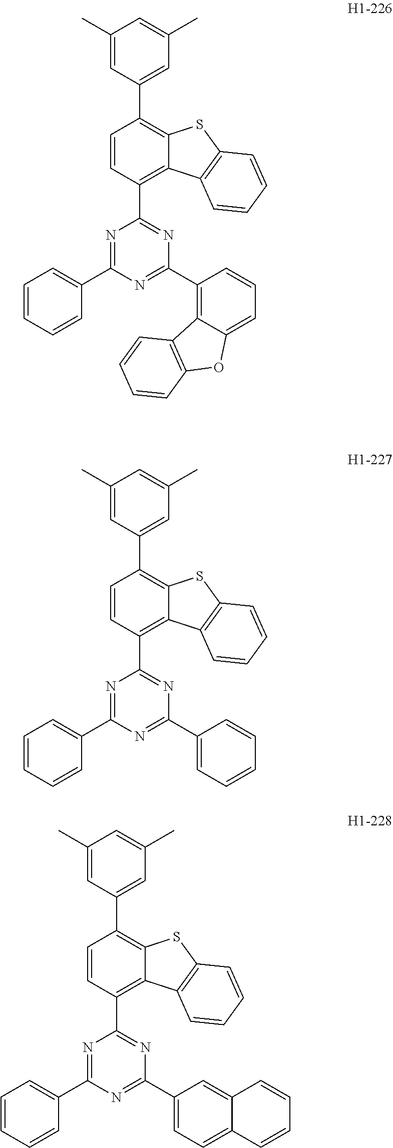

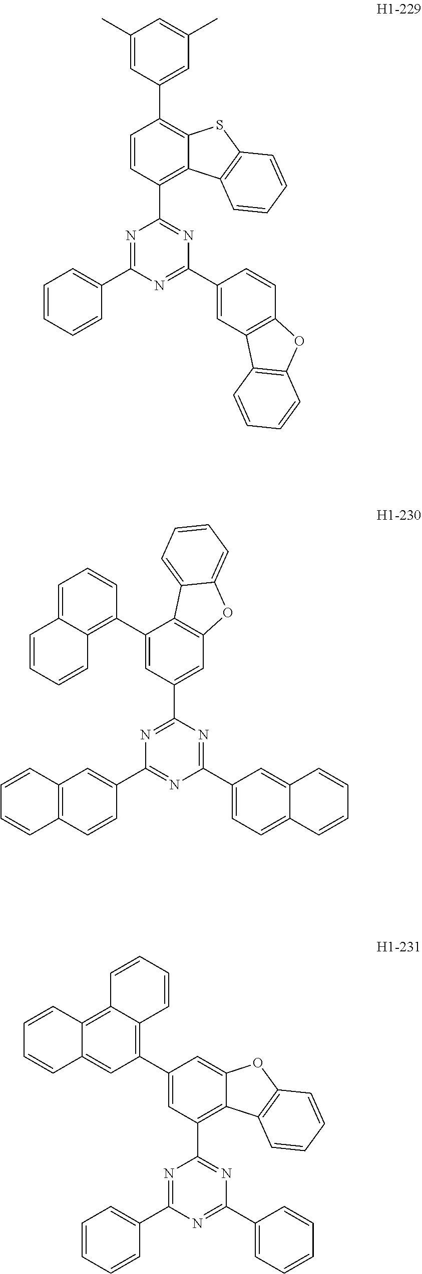 [SMILES: Cc1cc(C)cc(-c2ccc(-c3nc(-c4ccccc4)nc(-c4ccc5oc6ccccc6c5c4)n3)c3c2sc2ccccc23)c1 ; c1ccc2cc(-c3nc(-c4ccc5ccccc5c4)nc(-c4cc(-c5cccc6ccccc56)c5c(c4)oc4ccccc45)n3)ccc2c1 ; c1ccc(-c2nc(-c3ccccc3)nc(-c3cc(-c4cc5ccccc5c5ccccc45)cc4oc5ccccc5c34)n2)cc1]

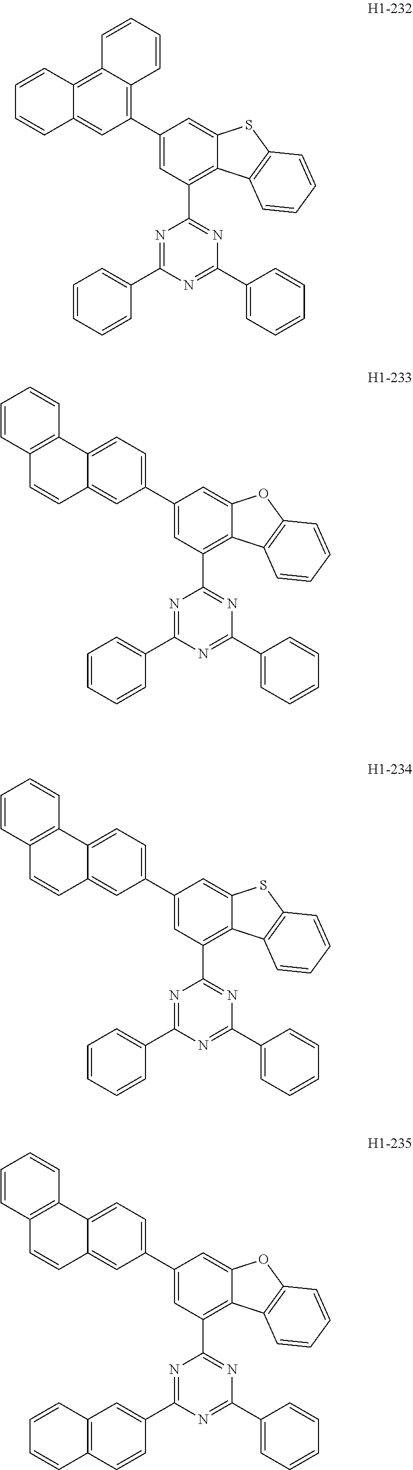 [SMILES: c1ccc(-c2nc(-c3ccccc3)nc(-c3cc(-c4cc5ccccc5c5ccccc45)cc4sc5ccccc5c34)n2)cc1 ; c1ccc(-c2nc(-c3ccccc3)nc(-c3cc(-c4ccc5c(ccc6ccccc65)c4)cc4oc5ccccc5c34)n2)cc1 ; c1ccc(-c2nc(-c3ccccc3)nc(-c3cc(-c4ccc5c(ccc6ccccc65)c4)cc4sc5ccccc5c34)n2)cc1 ; c1ccc(-c2nc(-c3ccc4ccccc4c3)nc(-c3cc(-c4ccc5c(ccc6ccccc65)c4)cc4oc5ccccc5c34)n2)cc1]

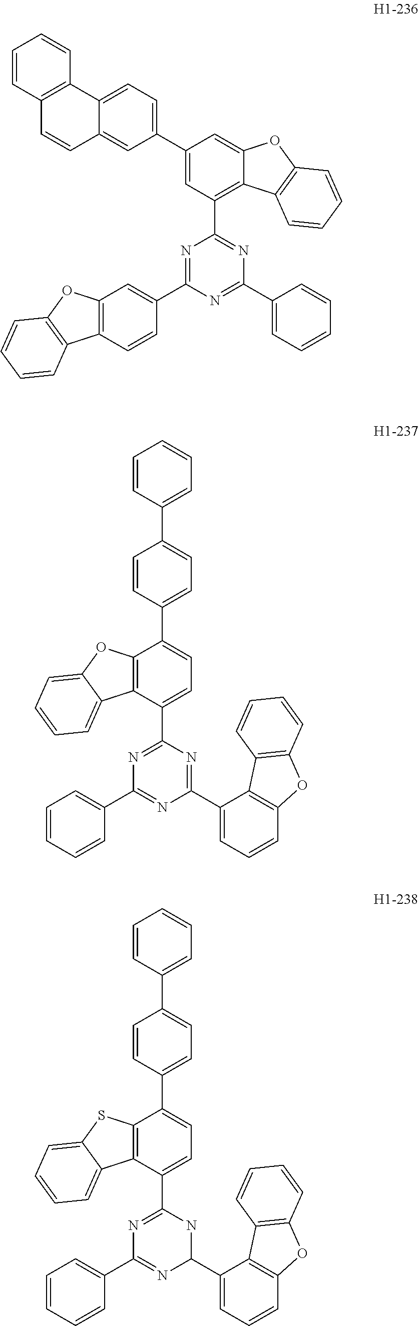 [SMILES: c1ccc(-c2nc(-c3ccc4c(c3)oc3ccccc34)nc(-c3cc(-c4ccc5c(ccc6ccccc65)c4)cc4oc5ccccc5c34)n2)cc1 ; c1ccc(-c2ccc(-c3ccc(-c4nc(-c5ccccc5)nc(-c5cccc6oc7ccccc7c56)n4)c4c3oc3ccccc34)cc2)cc1 ; c1ccc(C2=NC(c3cccc4oc5ccccc5c34)NC(c3ccc(-c4ccc(-c5ccccc5)cc4)c4sc5ccccc5c34)=N2)cc1]

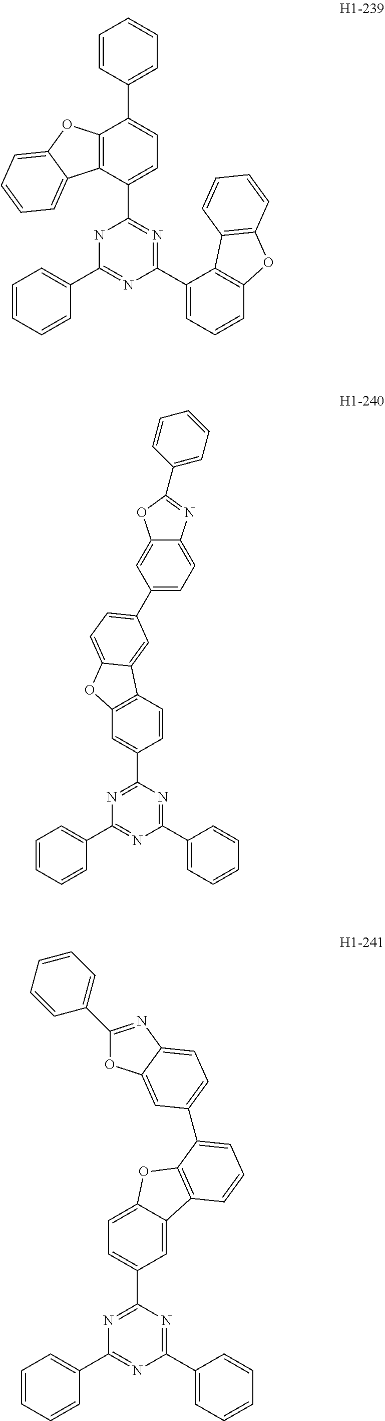

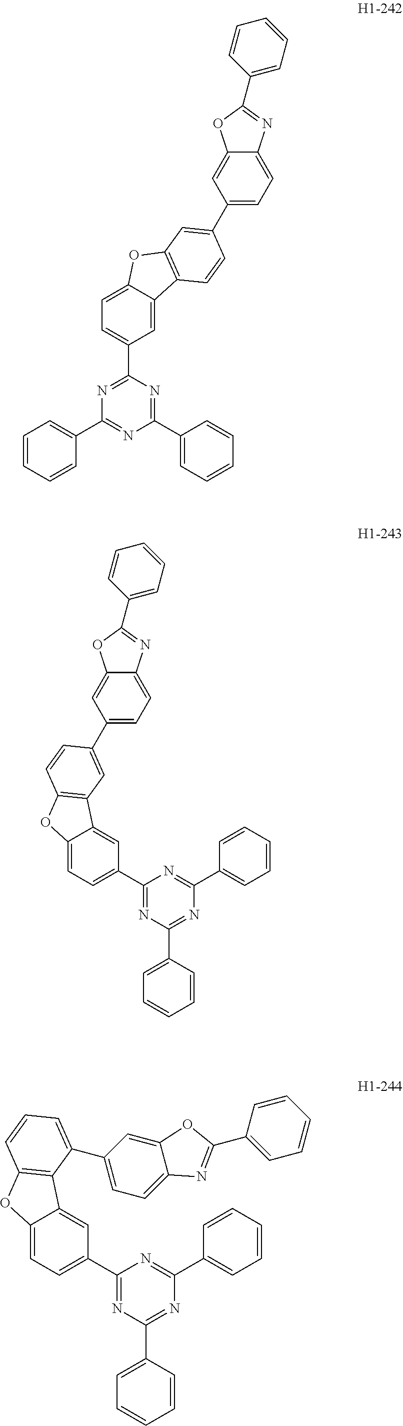 [SMILES: c1ccc(-c2nc(-c3ccccc3)nc(-c3ccc4oc5cc(-c6ccc7nc(-c8ccccc8)oc7c6)ccc5c4c3)n2)cc1 ; c1ccc(-c2nc(-c3ccccc3)nc(-c3ccc4oc5ccc(-c6ccc7nc(-c8ccccc8)oc7c6)cc5c4c3)n2)cc1 ; c1ccc(-c2nc(-c3ccccc3)nc(-c3ccc4oc5cccc(-c6ccc7nc(-c8ccccc8)oc7c6)c5c4c3)n2)cc1]

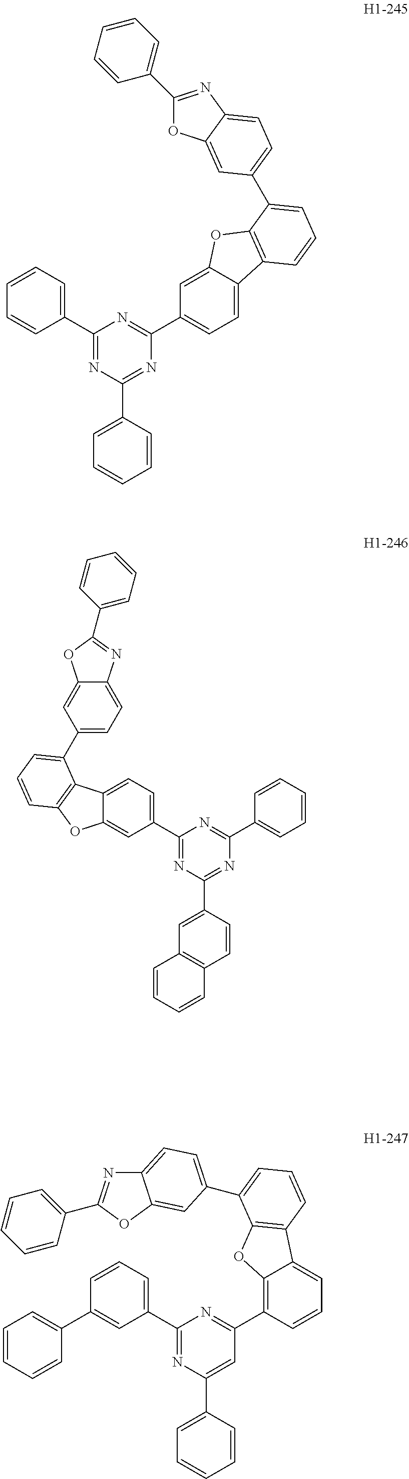 [SMILES: c1ccc(-c2nc(-c3ccccc3)nc(-c3ccc4c(c3)oc3c(-c5ccc6nc(-c7ccccc7)oc6c5)cccc34)n2)cc1 ; c1ccc(-c2nc(-c3ccc4ccccc4c3)nc(-c3ccc4c(c3)oc3cccc(-c5ccc6nc(-c7ccccc7)oc6c5)c34)n2)cc1 ; c1ccc(-c2cccc(-c3nc(-c4ccccc4)cc(-c4cccc5c4oc4c(-c6ccc7nc(-c8ccccc8)oc7c6)cccc45)n3)c2)cc1]

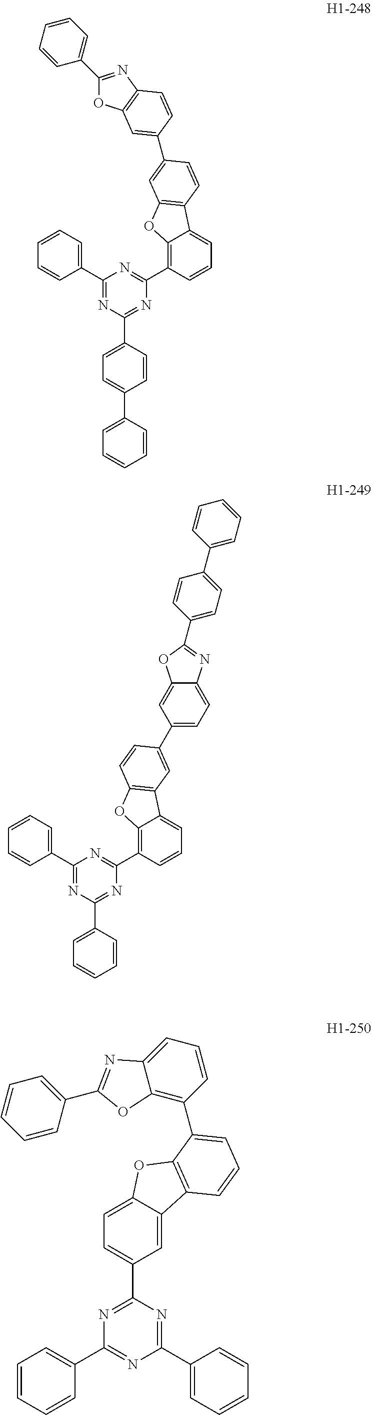 [SMILES: c1ccc(-c2ccc(-c3nc(-c4ccccc4)nc(-c4cccc5c4oc4cc(-c6ccc7nc(-c8ccccc8)oc7c6)ccc45)n3)cc2)cc1 ; c1ccc(-c2ccc(-c3nc4ccc(-c5ccc6oc7c(-c8nc(-c9ccccc9)nc(-c9ccccc9)n8)cccc7c6c5)cc4o3)cc2)cc1 ; c1ccc(-c2nc(-c3ccccc3)nc(-c3ccc4oc5c(-c6cccc7nc(-c8ccccc8)oc67)cccc5c4c3)n2)cc1]

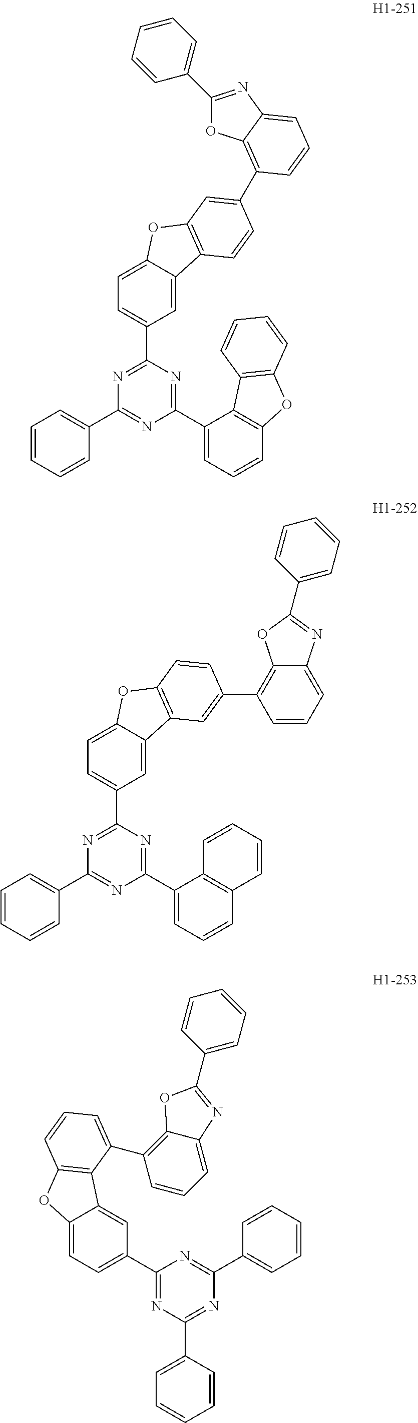 [SMILES: c1ccc(-c2nc(-c3ccc4oc5cc(-c6cccc7nc(-c8ccccc8)oc67)ccc5c4c3)nc(-c3cccc4oc5ccccc5c34)n2)cc1 ; c1ccc(-c2nc(-c3ccc4oc5ccc(-c6cccc7nc(-c8ccccc8)oc67)cc5c4c3)nc(-c3cccc4ccccc34)n2)cc1 ; c1ccc(-c2nc(-c3ccccc3)nc(-c3ccc4oc5cccc(-c6cccc7nc(-c8ccccc8)oc67)c5c4c3)n2)cc1]

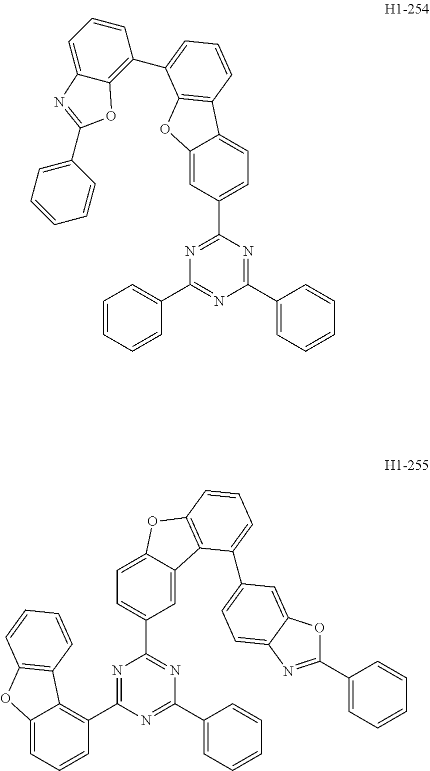 [SMILES: c1ccc(-c2nc(-c3ccccc3)nc(-c3ccc4c(c3)oc3c(-c5cccc6nc(-c7ccccc7)oc56)cccc34)n2)cc1 ; c1ccc(-c2nc(-c3ccc4oc5cccc(-c6ccc7nc(-c8ccccc8)oc7c6)c5c4c3)nc(-c3cccc4oc5ccccc5c34)n2)cc1]

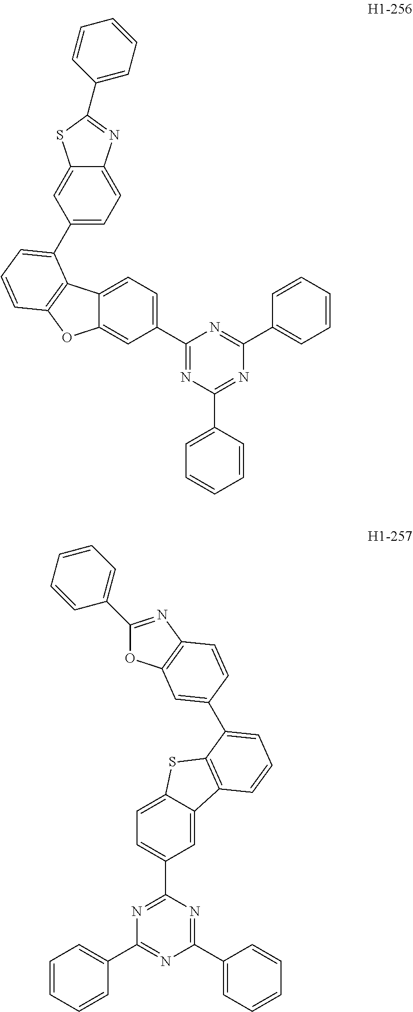 [SMILES: c1ccc(-c2nc(-c3ccccc3)nc(-c3ccc4c(c3)oc3cccc(-c5ccc6nc(-c7ccccc7)sc6c5)c34)n2)cc1 ; c1ccc(-c2nc(-c3ccccc3)nc(-c3ccc4sc5c(-c6ccc7nc(-c8ccccc8)oc7c6)cccc5c4c3)n2)cc1]

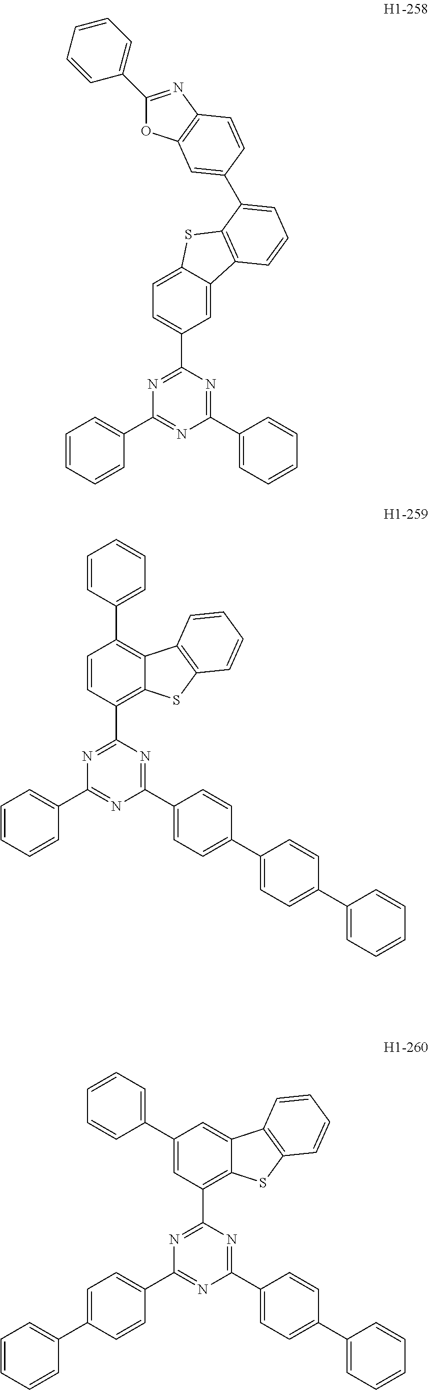 [SMILES: c1ccc(-c2nc(-c3ccccc3)nc(-c3ccc4sc5c(-c6ccc7nc(-c8ccccc8)oc7c6)cccc5c4c3)n2)cc1 ; c1ccc(-c2ccc(-c3ccc(-c4nc(-c5ccccc5)nc(-c5ccc(-c6ccccc6)c6c5sc5ccccc56)n4)cc3)cc2)cc1 ; c1ccc(-c2ccc(-c3nc(-c4ccc(-c5ccccc5)cc4)nc(-c4cc(-c5ccccc5)cc5c4sc4ccccc45)n3)cc2)cc1]

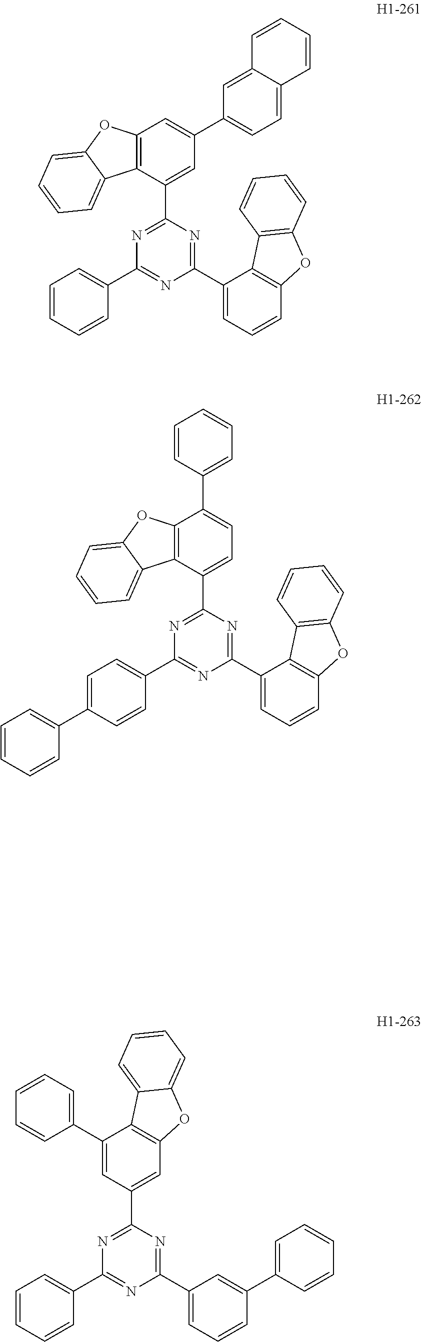 [SMILES: c1ccc(-c2nc(-c3cccc4oc5ccccc5c34)nc(-c3cc(-c4ccc5ccccc5c4)cc4oc5ccccc5c34)n2)cc1 ; c1ccc(-c2ccc(-c3nc(-c4cccc5oc6ccccc6c45)nc(-c4ccc(-c5ccccc5)c5oc6ccccc6c45)n3)cc2)cc1 ; c1ccc(-c2cccc(-c3nc(-c4ccccc4)nc(-c4cc(-c5ccccc5)c5c(c4)oc4ccccc45)n3)c2)cc1]

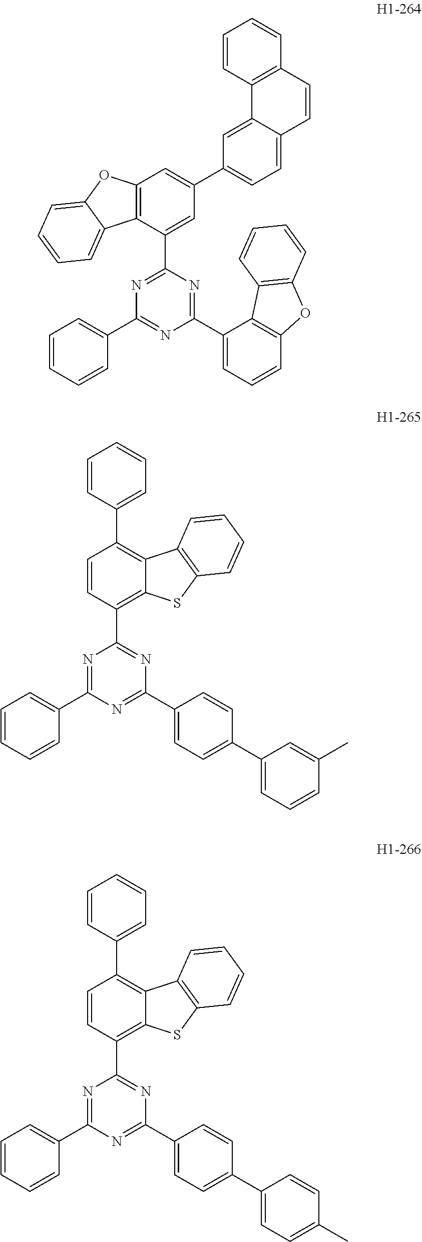 [SMILES: c1ccc(-c2nc(-c3cccc4oc5ccccc5c34)nc(-c3cc(-c4ccc5ccc6ccccc6c5c4)cc4oc5ccccc5c34)n2)cc1 ; Cc1cccc(-c2ccc(-c3nc(-c4ccccc4)nc(-c4ccc(-c5ccccc5)c5c4sc4ccccc45)n3)cc2)c1 ; Cc1ccc(-c2ccc(-c3nc(-c4ccccc4)nc(-c4ccc(-c5ccccc5)c5c4sc4ccccc45)n3)cc2)cc1]

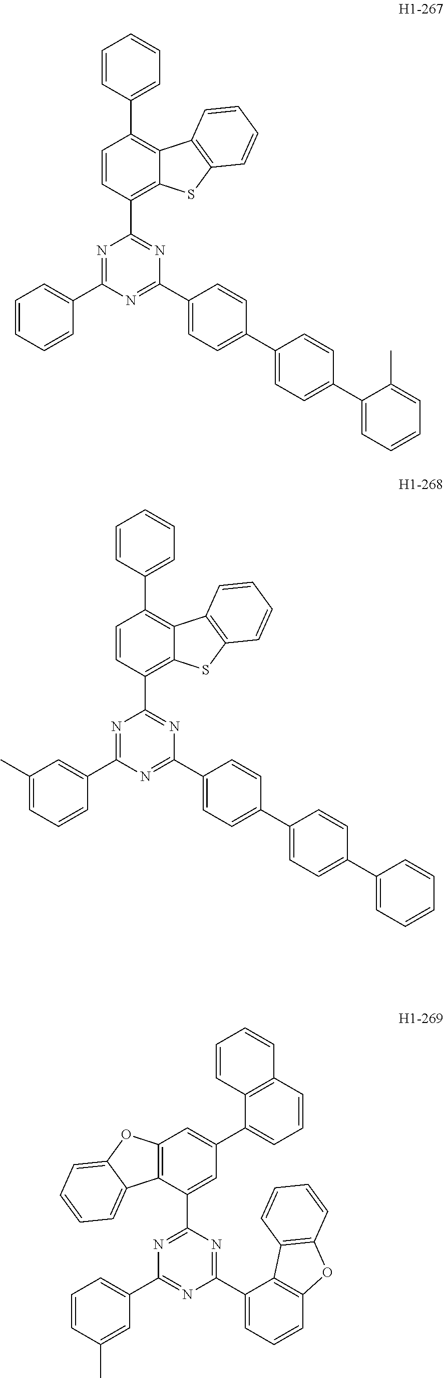 [SMILES: Cc1ccccc1-c1ccc(-c2ccc(-c3nc(-c4ccccc4)nc(-c4ccc(-c5ccccc5)c5c4sc4ccccc45)n3)cc2)cc1 ; Cc1cccc(-c2nc(-c3ccc(-c4ccc(-c5ccccc5)cc4)cc3)nc(-c3ccc(-c4ccccc4)c4c3sc3ccccc34)n2)c1 ; Cc1cccc(-c2nc(-c3cccc4oc5ccccc5c34)nc(-c3cc(-c4cccc5ccccc45)cc4oc5ccccc5c34)n2)c1]

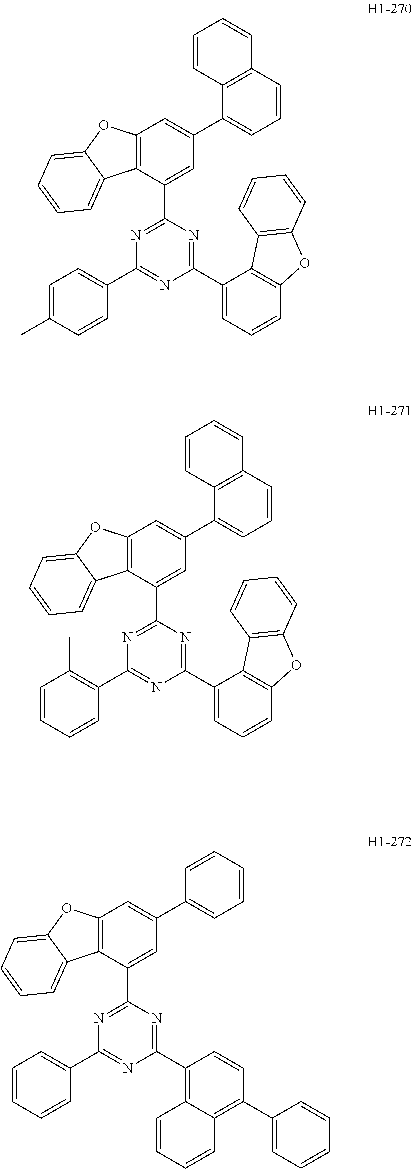 [SMILES: Cc1ccc(-c2nc(-c3cccc4oc5ccccc5c34)nc(-c3cc(-c4cccc5ccccc45)cc4oc5ccccc5c34)n2)cc1 ; Cc1ccccc1-c1nc(-c2cccc3oc4ccccc4c23)nc(-c2cc(-c3cccc4ccccc34)cc3oc4ccccc4c23)n1 ; c1ccc(-c2cc(-c3nc(-c4ccccc4)nc(-c4ccc(-c5ccccc5)c5ccccc45)n3)c3c(c2)oc2ccccc23)cc1]

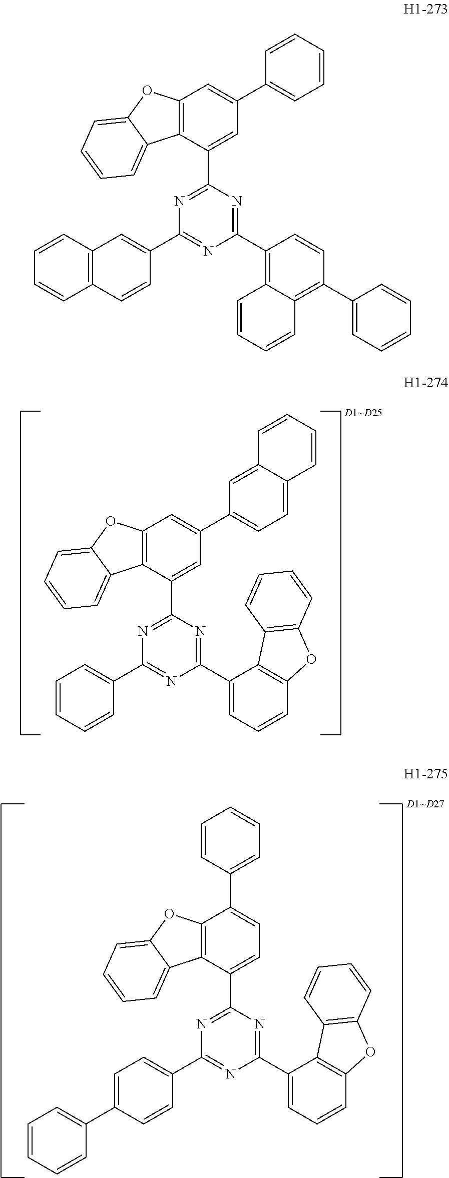

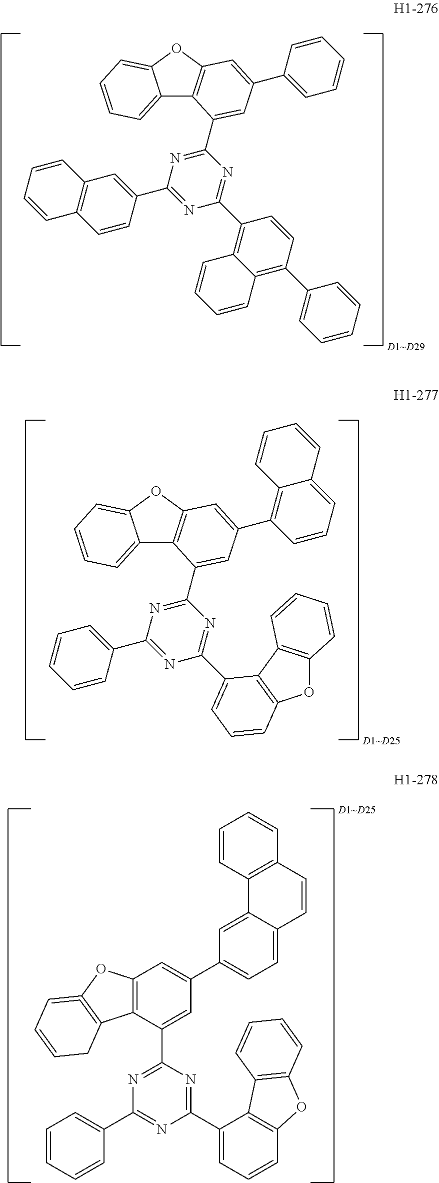

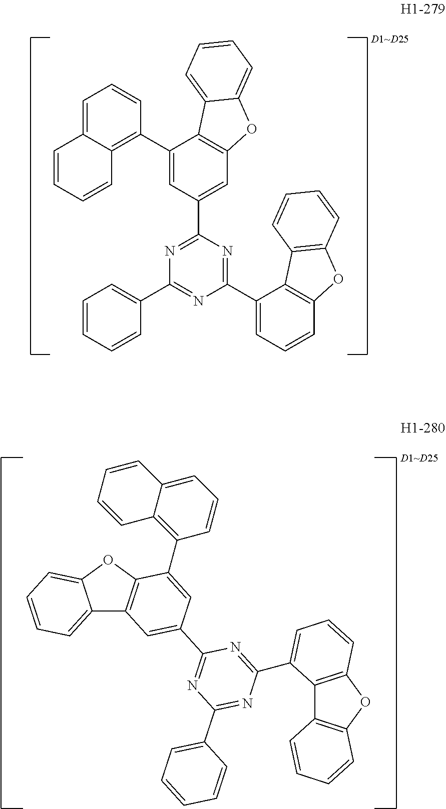

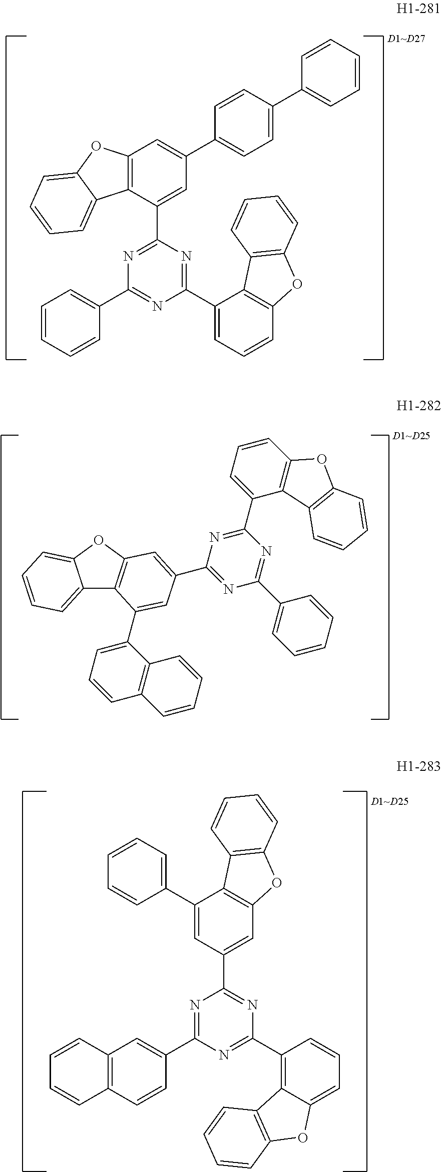

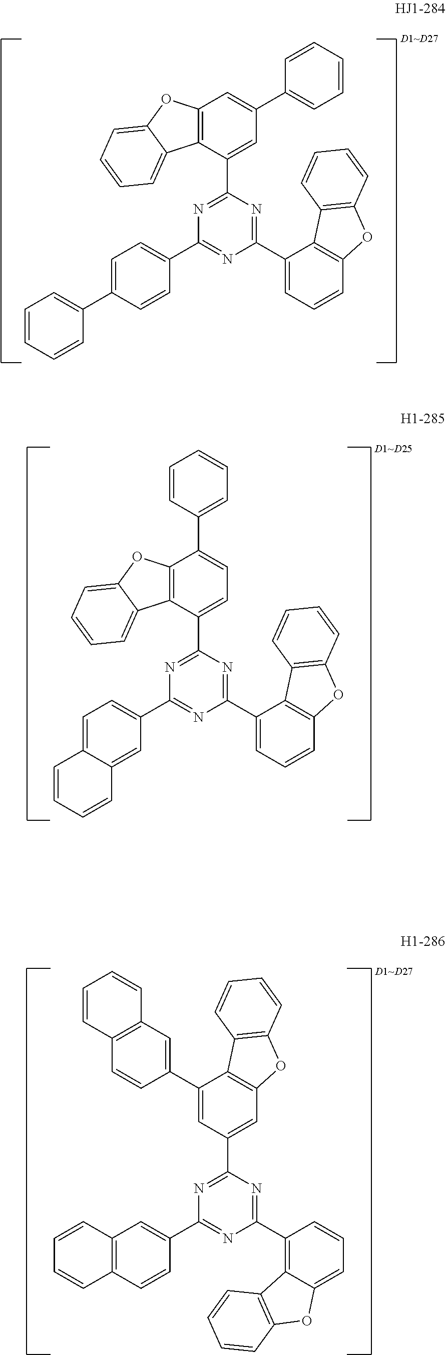

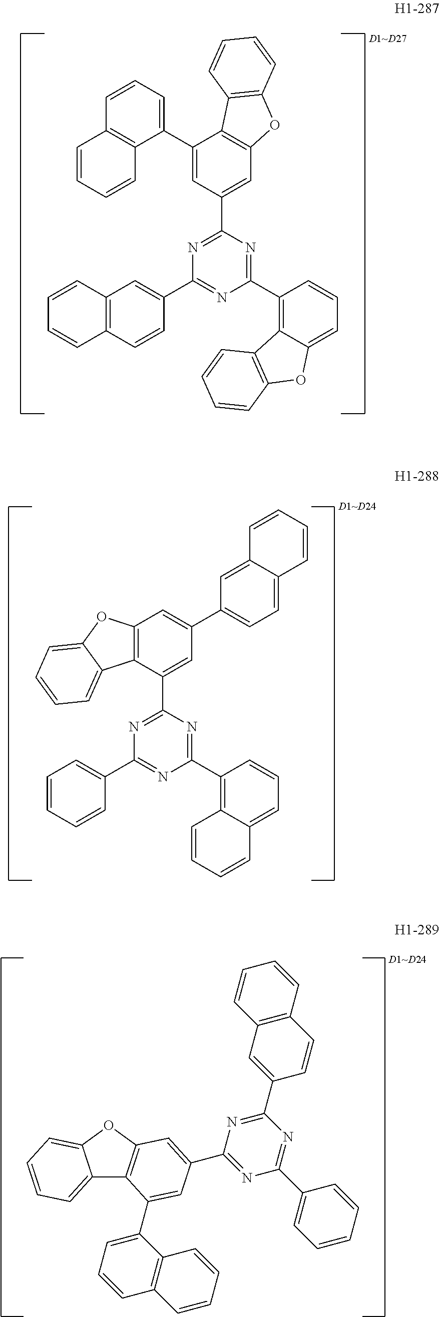

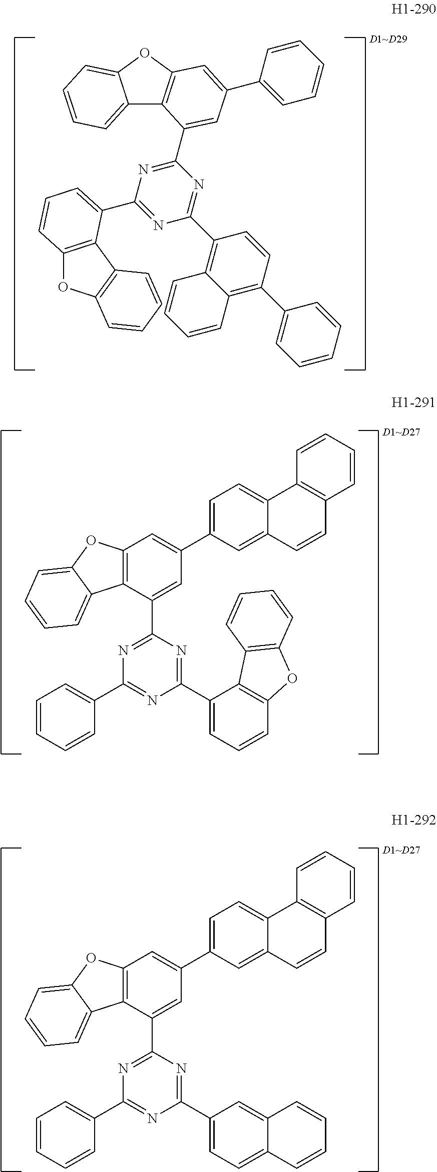

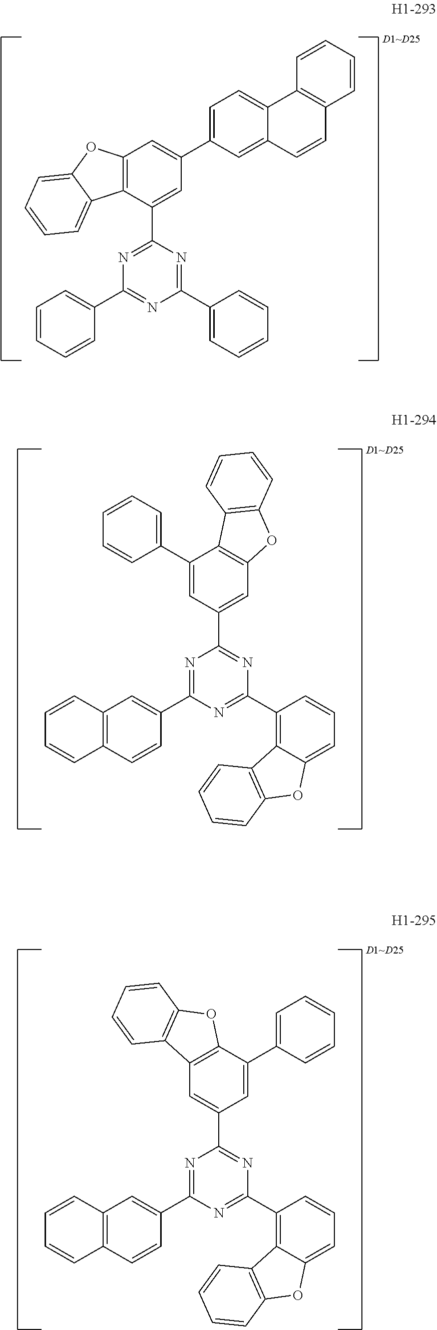

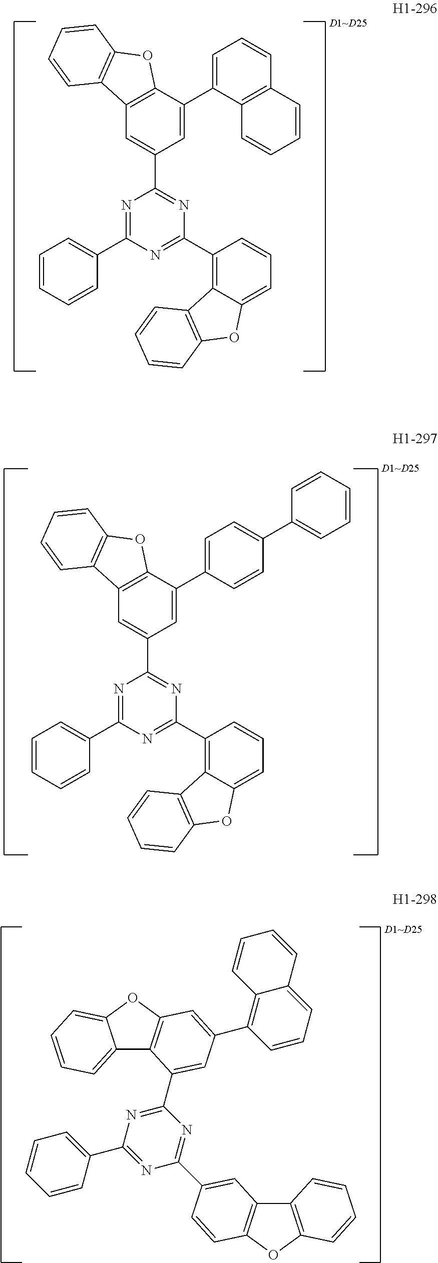

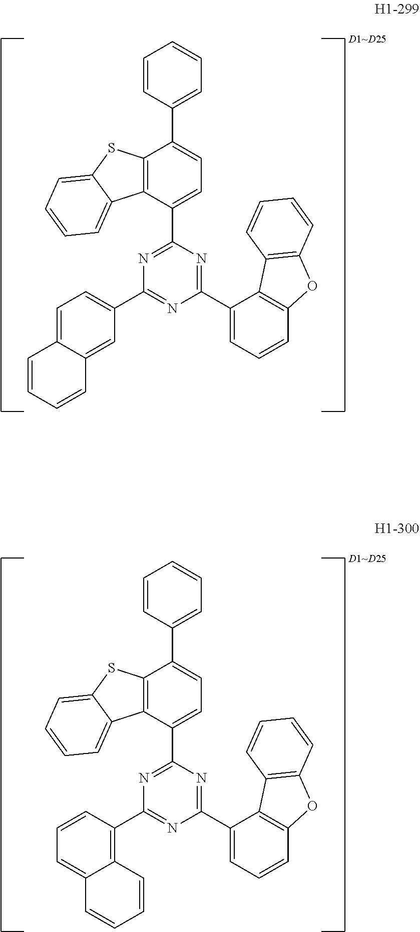

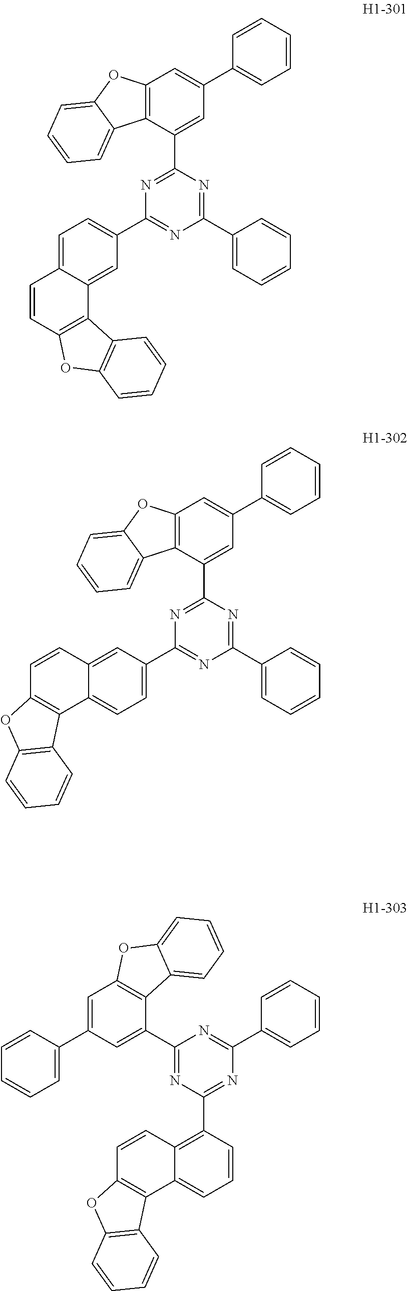 [SMILES: c1ccc(-c2cc(-c3nc(-c4ccccc4)nc(-c4ccc5ccc6oc7ccccc7c6c5c4)n3)c3c(c2)oc2ccccc23)cc1 ; c1ccc(-c2cc(-c3nc(-c4ccccc4)nc(-c4ccc5c(ccc6oc7ccccc7c65)c4)n3)c3c(c2)oc2ccccc23)cc1 ; c1ccc(-c2cc(-c3nc(-c4ccccc4)nc(-c4cccc5c4ccc4oc6ccccc6c45)n3)c3c(c2)oc2ccccc23)cc1]

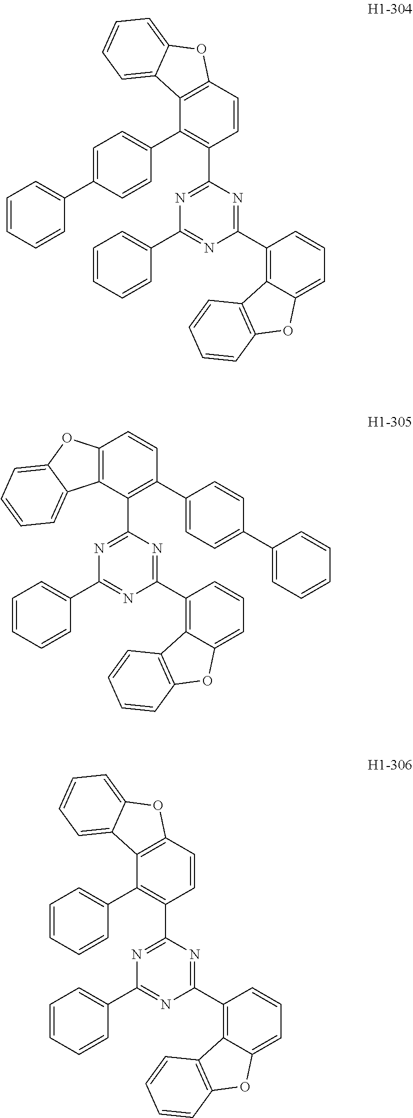 [SMILES: c1ccc(-c2ccc(-c3c(-c4nc(-c5ccccc5)nc(-c5cccc6oc7ccccc7c56)n4)ccc4oc5ccccc5c34)cc2)cc1 ; c1ccc(-c2ccc(-c3ccc4oc5ccccc5c4c3-c3nc(-c4ccccc4)nc(-c4cccc5oc6ccccc6c45)n3)cc2)cc1 ; c1ccc(-c2nc(-c3ccc4oc5ccccc5c4c3-c3ccccc3)nc(-c3cccc4oc5ccccc5c34)n2)cc1]

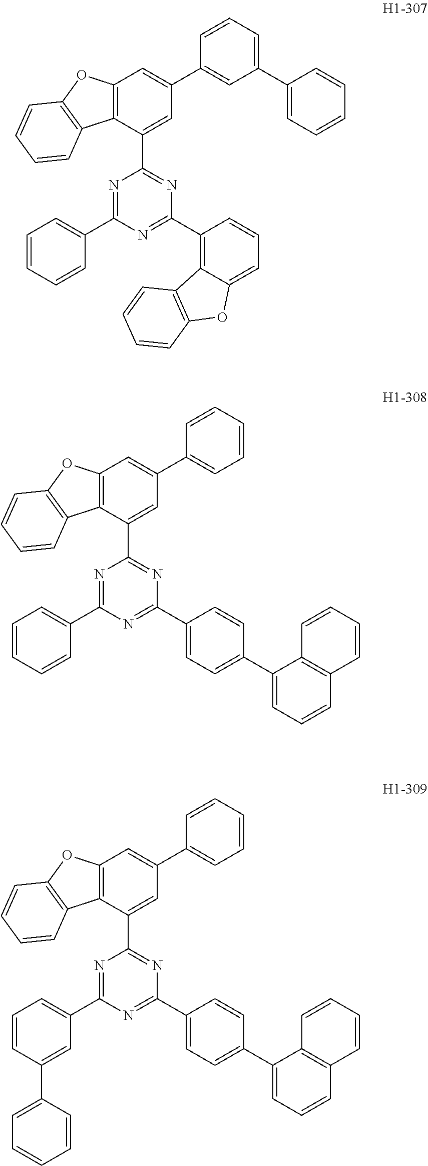 [SMILES: c1ccc(-c2cccc(-c3cc(-c4nc(-c5ccccc5)nc(-c5cccc6oc7ccccc7c56)n4)c4c(c3)oc3ccccc34)c2)cc1 ; c1ccc(-c2cc(-c3nc(-c4ccccc4)nc(-c4ccc(-c5cccc6ccccc56)cc4)n3)c3c(c2)oc2ccccc23)cc1 ; c1ccc(-c2cccc(-c3nc(-c4ccc(-c5cccc6ccccc56)cc4)nc(-c4cc(-c5ccccc5)cc5oc6ccccc6c45)n3)c2)cc1]

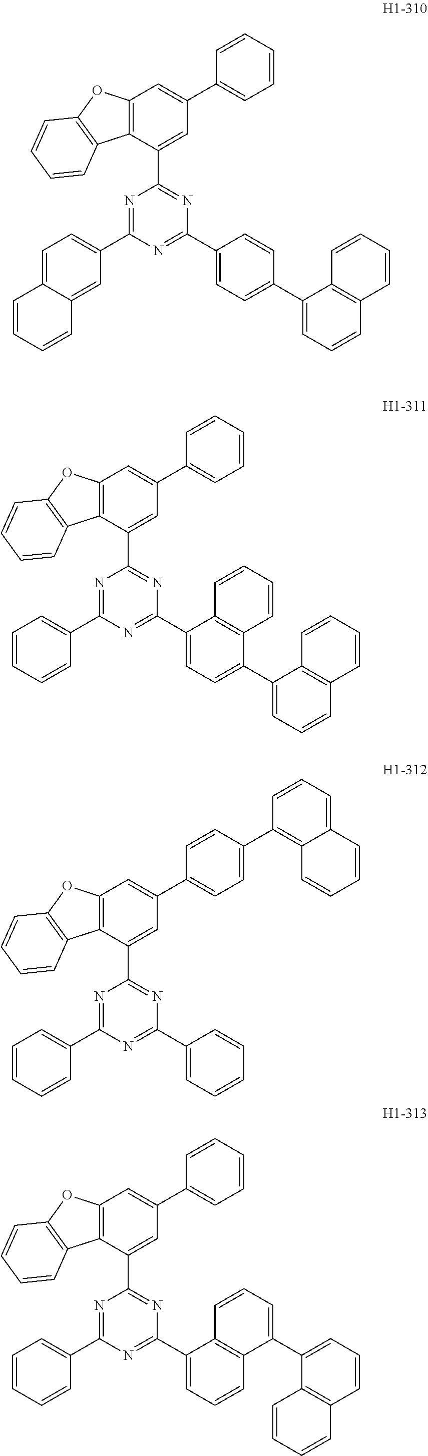

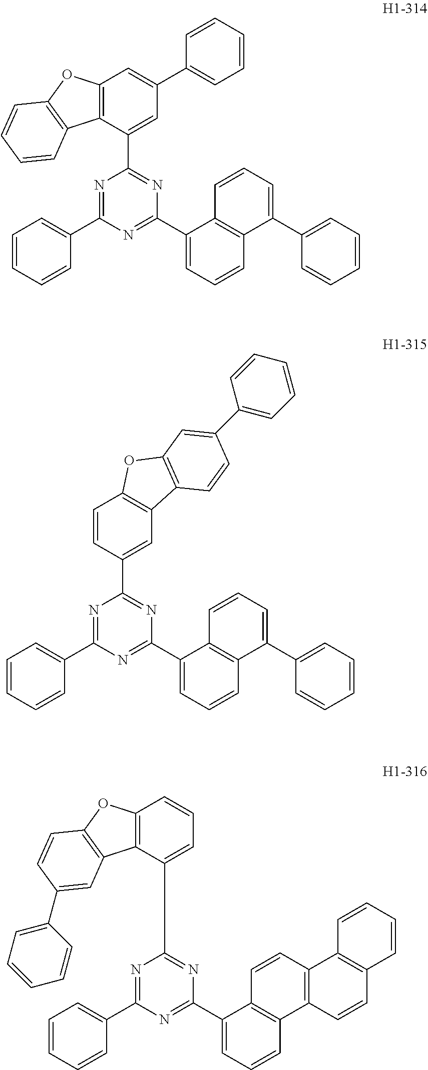 [SMILES: c1ccc(-c2cc(-c3nc(-c4ccccc4)nc(-c4cccc5c(-c6ccccc6)cccc45)n3)c3c(c2)oc2ccccc23)cc1 ; c1ccc(-c2ccc3c(c2)oc2ccc(-c4nc(-c5ccccc5)nc(-c5cccc6c(-c7ccccc7)cccc56)n4)cc23)cc1 ; c1ccc(-c2ccc3oc4cccc(-c5nc(-c6ccccc6)nc(-c6cccc7c6ccc6c8ccccc8ccc76)n5)c4c3c2)cc1]

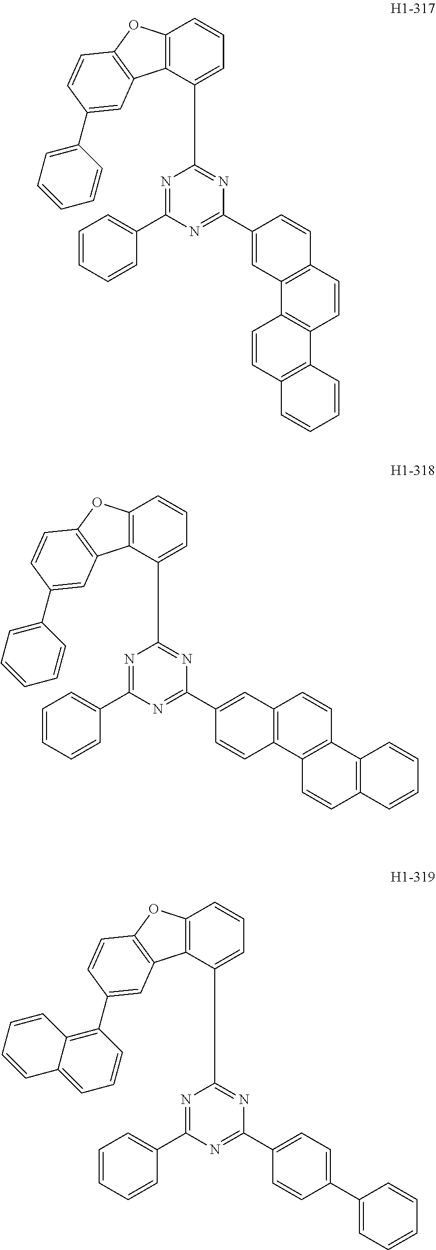 [SMILES: c1ccc(-c2ccc3oc4cccc(-c5nc(-c6ccccc6)nc(-c6ccc7ccc8c9ccccc9ccc8c7c6)n5)c4c3c2)cc1 ; c1ccc(-c2ccc3oc4cccc(-c5nc(-c6ccccc6)nc(-c6ccc7c(ccc8c9ccccc9ccc78)c6)n5)c4c3c2)cc1 ; c1ccc(-c2ccc(-c3nc(-c4ccccc4)nc(-c4cccc5oc6ccc(-c7cccc8ccccc78)cc6c45)n3)cc2)cc1]

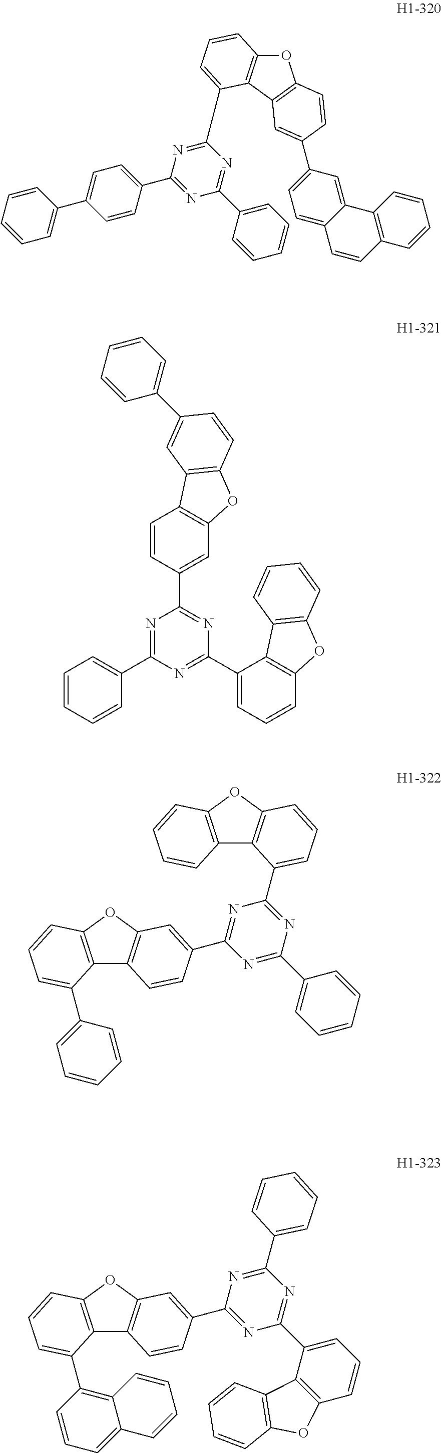 [SMILES: c1ccc(-c2ccc(-c3nc(-c4ccccc4)nc(-c4cccc5oc6ccc(-c7ccc8ccc9ccccc9c8c7)cc6c45)n3)cc2)cc1 ; c1ccc(-c2ccc3oc4cc(-c5nc(-c6ccccc6)nc(-c6cccc7oc8ccccc8c67)n5)ccc4c3c2)cc1 ; c1ccc(-c2nc(-c3ccc4c(c3)oc3cccc(-c5ccccc5)c34)nc(-c3cccc4oc5ccccc5c34)n2)cc1 ; c1ccc(-c2nc(-c3ccc4c(c3)oc3cccc(-c5cccc6ccccc56)c34)nc(-c3cccc4oc5ccccc5c34)n2)cc1]

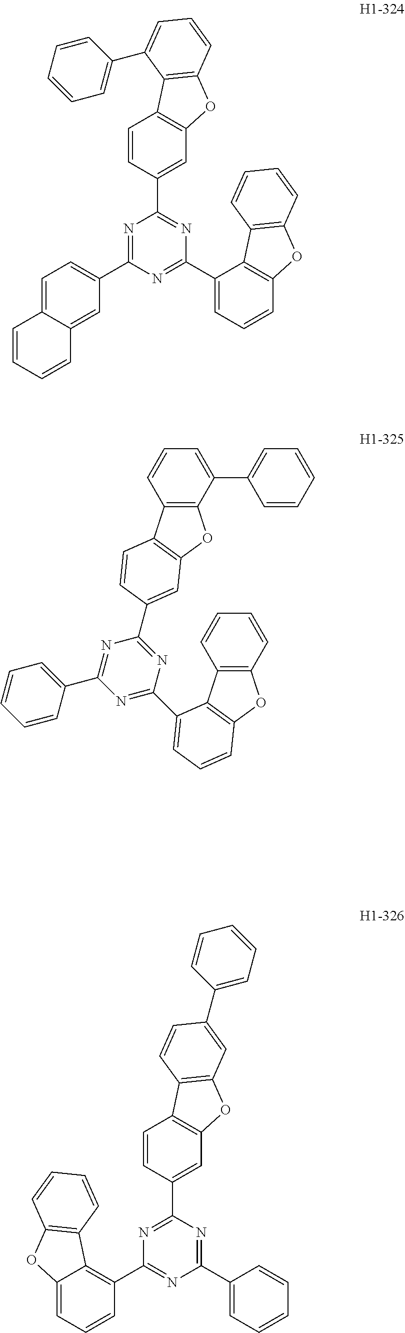 [SMILES: c1ccc(-c2cccc3oc4cc(-c5nc(-c6ccc7ccccc7c6)nc(-c6cccc7oc8ccccc8c67)n5)ccc4c23)cc1 ; c1ccc(-c2nc(-c3ccc4c(c3)oc3c(-c5ccccc5)cccc34)nc(-c3cccc4oc5ccccc5c34)n2)cc1 ; c1ccc(-c2ccc3c(c2)oc2cc(-c4nc(-c5ccccc5)nc(-c5cccc6oc7ccccc7c56)n4)ccc23)cc1]

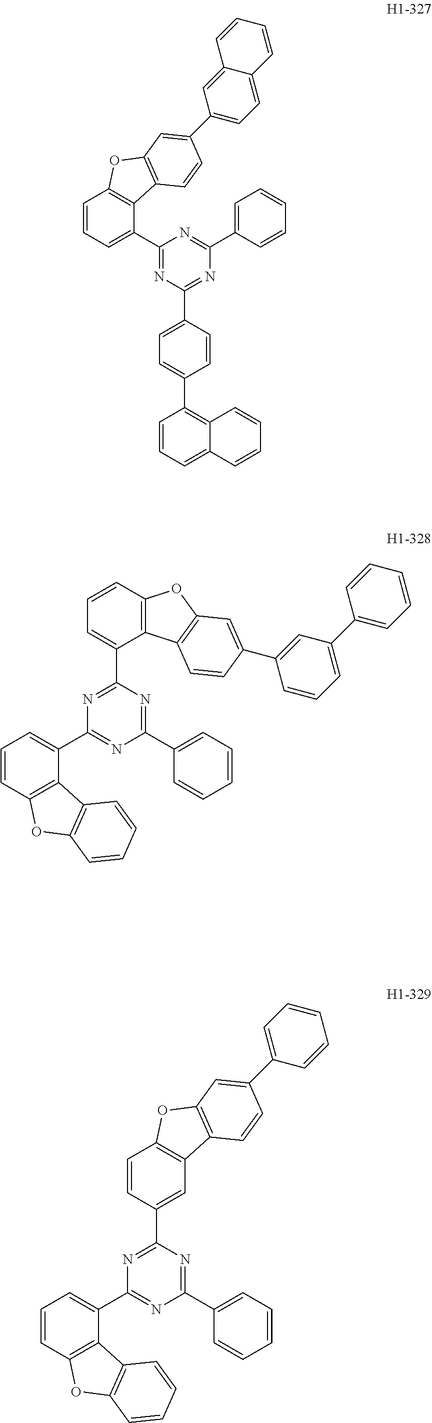 [SMILES: c1ccc(-c2nc(-c3ccc(-c4cccc5ccccc45)cc3)nc(-c3cccc4oc5cc(-c6ccc7ccccc7c6)ccc5c34)n2)cc1 ; c1ccc(-c2cccc(-c3ccc4c(c3)oc3cccc(-c5nc(-c6ccccc6)nc(-c6cccc7oc8ccccc8c67)n5)c34)c2)cc1 ; c1ccc(-c2ccc3c(c2)oc2ccc(-c4nc(-c5ccccc5)nc(-c5cccc6oc7ccccc7c56)n4)cc23)cc1]

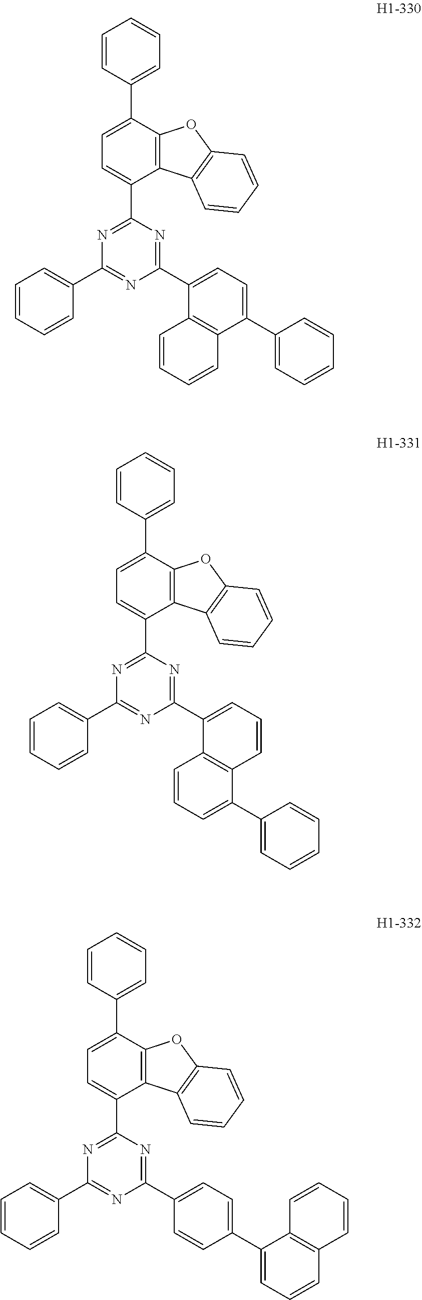 [SMILES: c1ccc(-c2nc(-c3ccc(-c4ccccc4)c4ccccc34)nc(-c3ccc(-c4ccccc4)c4oc5ccccc5c34)n2)cc1 ; c1ccc(-c2nc(-c3cccc4c(-c5ccccc5)cccc34)nc(-c3ccc(-c4ccccc4)c4oc5ccccc5c34)n2)cc1 ; c1ccc(-c2nc(-c3ccc(-c4cccc5ccccc45)cc3)nc(-c3ccc(-c4ccccc4)c4oc5ccccc5c34)n2)cc1]

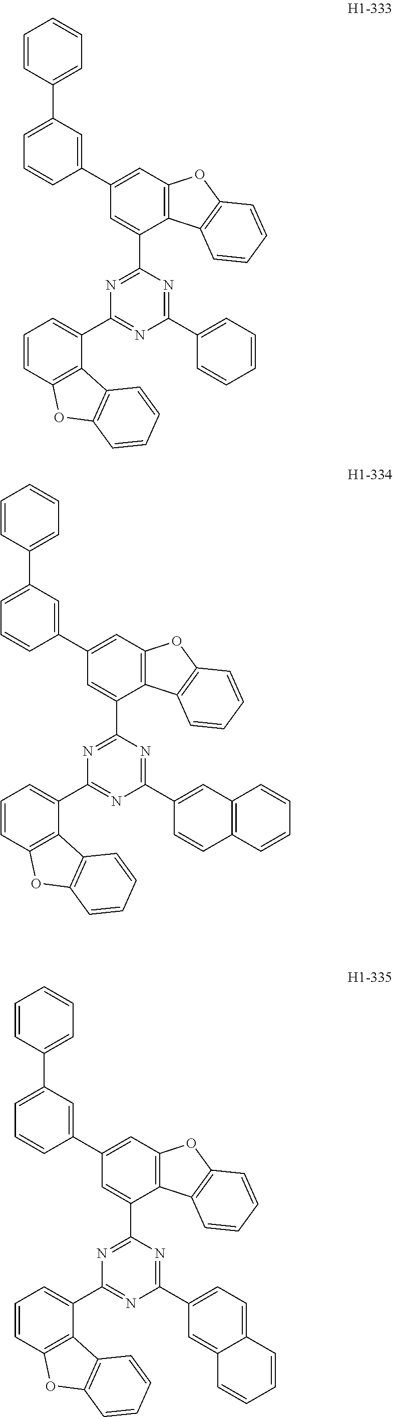 [SMILES: c1ccc(-c2cccc(-c3cc(-c4nc(-c5ccccc5)nc(-c5cccc6oc7ccccc7c56)n4)c4c(c3)oc3ccccc34)c2)cc1 ; c1ccc(-c2cccc(-c3cc(-c4nc(-c5ccc6ccccc6c5)nc(-c5cccc6oc7ccccc7c56)n4)c4c(c3)oc3ccccc34)c2)cc1 ; c1ccc(-c2cccc(-c3cc(-c4nc(-c5ccc6ccccc6c5)nc(-c5cccc6oc7ccccc7c56)n4)c4c(c3)oc3ccccc34)c2)cc1]

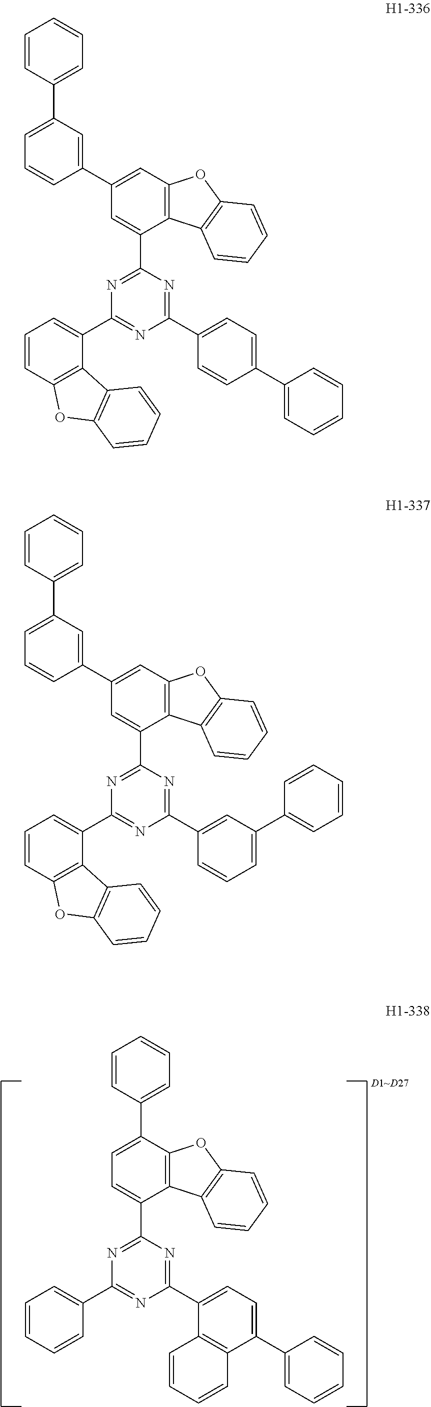

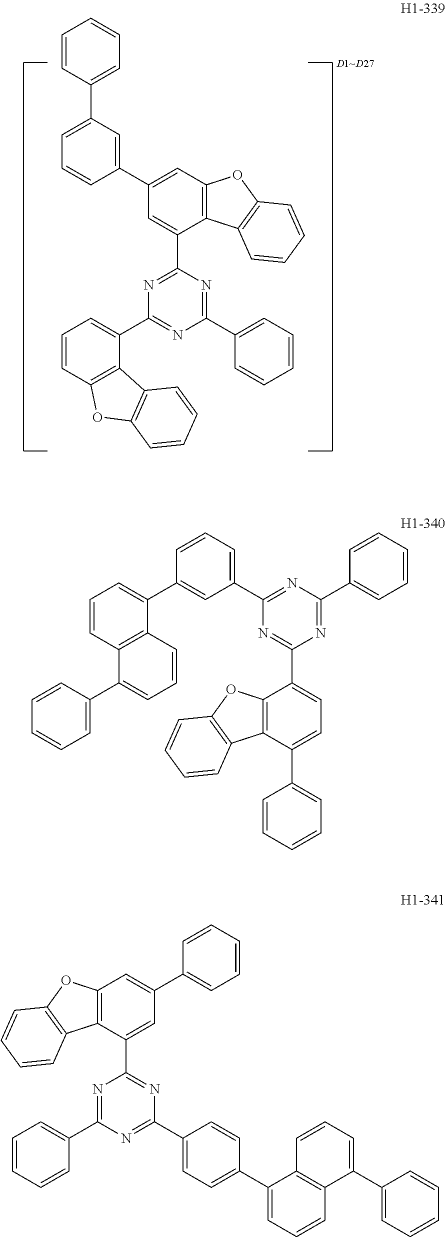

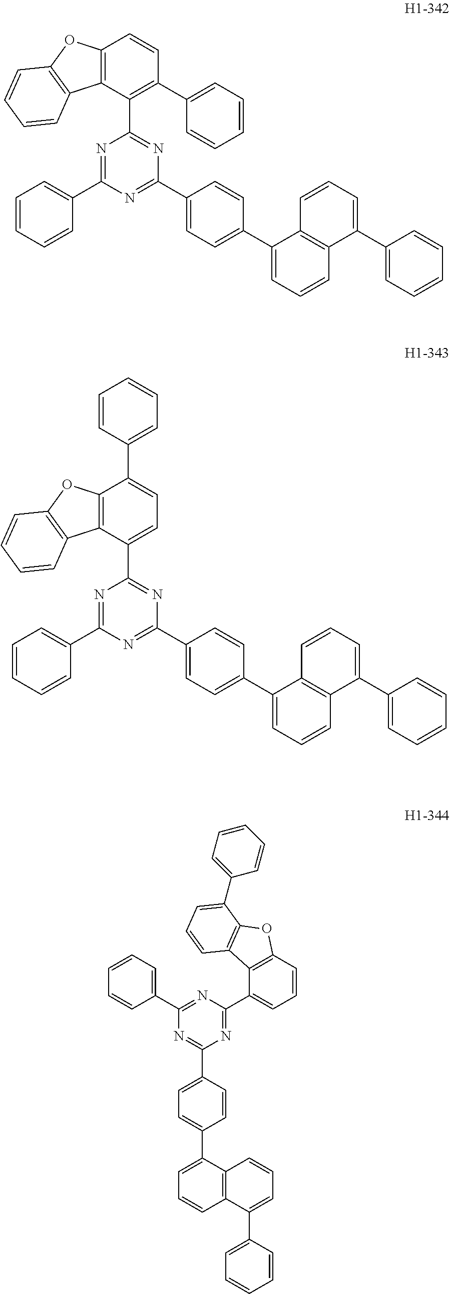 [SMILES: c1ccc(-c2nc(-c3ccc(-c4cccc5c(-c6ccccc6)cccc45)cc3)nc(-c3c(-c4ccccc4)ccc4oc5ccccc5c34)n2)cc1 ; c1ccc(-c2nc(-c3ccc(-c4cccc5c(-c6ccccc6)cccc45)cc3)nc(-c3ccc(-c4ccccc4)c4oc5ccccc5c34)n2)cc1 ; c1ccc(-c2nc(-c3ccc(-c4cccc5c(-c6ccccc6)cccc45)cc3)nc(-c3cccc4oc5c(-c6ccccc6)cccc5c34)n2)cc1]

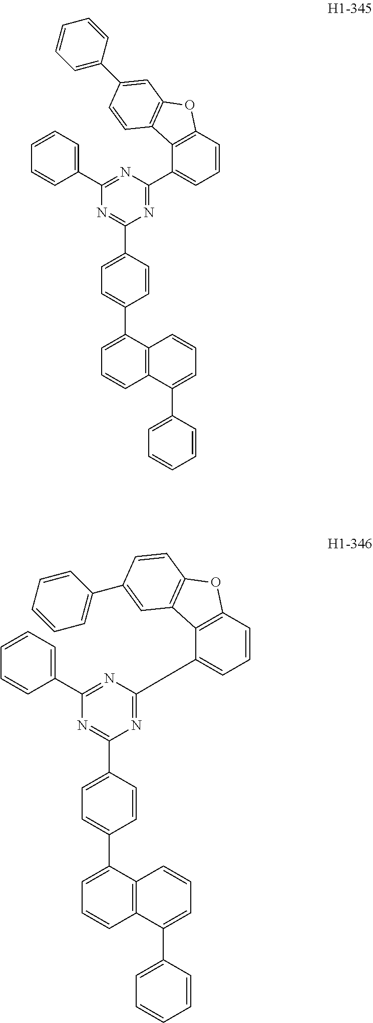 [SMILES: c1ccc(-c2ccc3c(c2)oc2cccc(-c4nc(-c5ccccc5)nc(-c5ccc(-c6cccc7c(-c8ccccc8)cccc67)cc5)n4)c23)cc1 ; c1ccc(-c2ccc3oc4cccc(-c5nc(-c6ccccc6)nc(-c6ccc(-c7cccc8c(-c9ccccc9)cccc78)cc6)n5)c4c3c2)cc1]

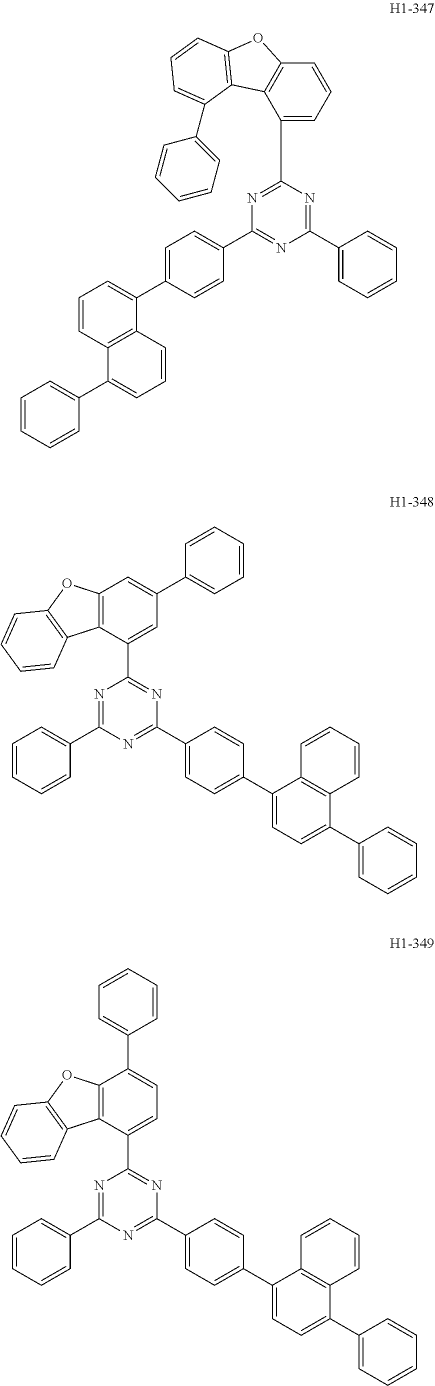 [SMILES: c1ccc(-c2nc(-c3ccc(-c4cccc5c(-c6ccccc6)cccc45)cc3)nc(-c3cccc4oc5cccc(-c6ccccc6)c5c34)n2)cc1 ; c1ccc(-c2cc(-c3nc(-c4ccccc4)nc(-c4ccc(-c5ccc(-c6ccccc6)c6ccccc56)cc4)n3)c3c(c2)oc2ccccc23)cc1 ; c1ccc(-c2nc(-c3ccc(-c4ccc(-c5ccccc5)c5ccccc45)cc3)nc(-c3ccc(-c4ccccc4)c4oc5ccccc5c34)n2)cc1]

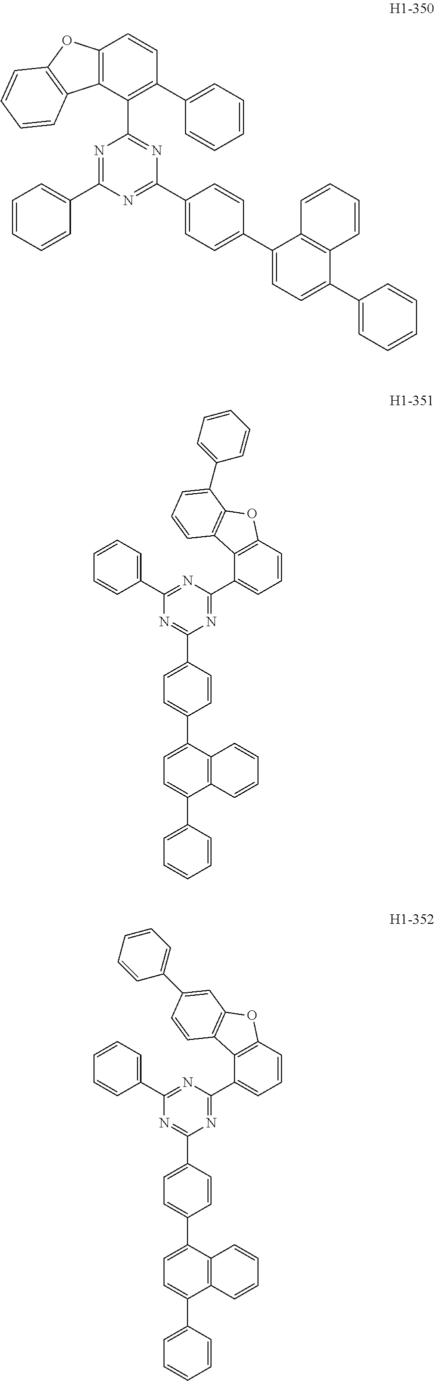 [SMILES: c1ccc(-c2nc(-c3ccc(-c4ccc(-c5ccccc5)c5ccccc45)cc3)nc(-c3c(-c4ccccc4)ccc4oc5ccccc5c34)n2)cc1 ; c1ccc(-c2nc(-c3ccc(-c4ccc(-c5ccccc5)c5ccccc45)cc3)nc(-c3cccc4oc5c(-c6ccccc6)cccc5c34)n2)cc1 ; c1ccc(-c2ccc3c(c2)oc2cccc(-c4nc(-c5ccccc5)nc(-c5ccc(-c6ccc(-c7ccccc7)c7ccccc67)cc5)n4)c23)cc1]

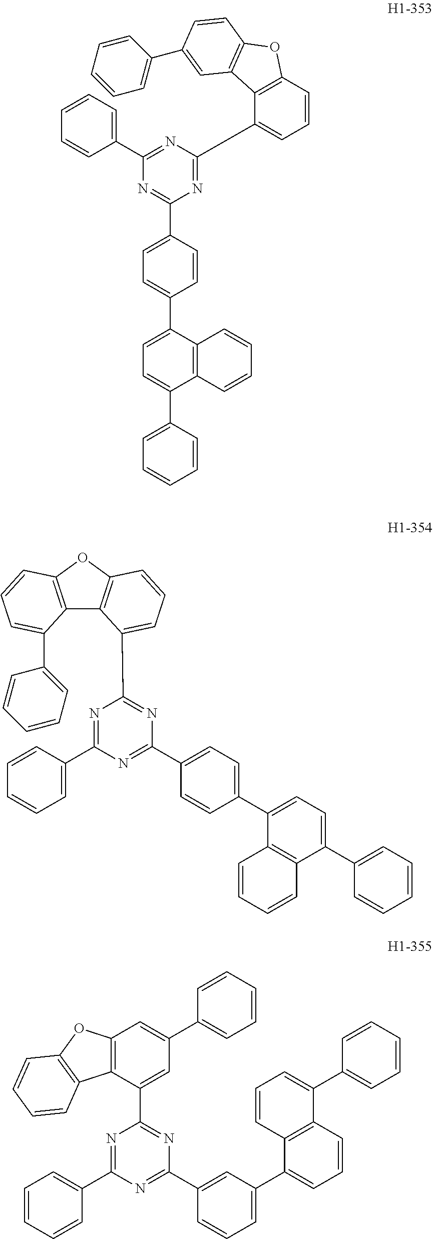 [SMILES: c1ccc(-c2ccc3oc4cccc(-c5nc(-c6ccccc6)nc(-c6ccc(-c7ccc(-c8ccccc8)c8ccccc78)cc6)n5)c4c3c2)cc1 ; c1ccc(-c2nc(-c3ccc(-c4ccc(-c5ccccc5)c5ccccc45)cc3)nc(-c3cccc4oc5cccc(-c6ccccc6)c5c34)n2)cc1 ; c1ccc(-c2cc(-c3nc(-c4ccccc4)nc(-c4cccc(-c5cccc6c(-c7ccccc7)cccc56)c4)n3)c3c(c2)oc2ccccc23)cc1]

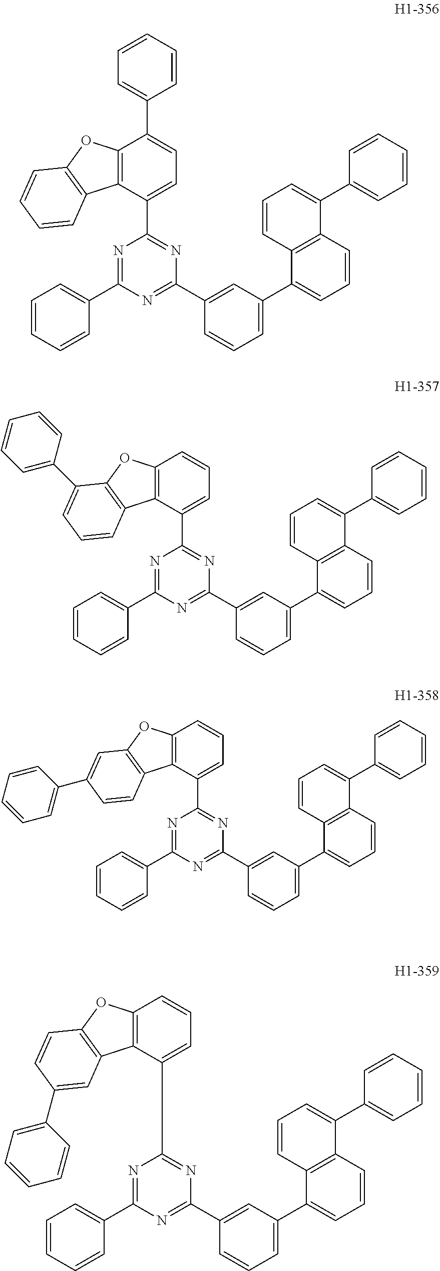 [SMILES: c1ccc(-c2nc(-c3cccc(-c4cccc5c(-c6ccccc6)cccc45)c3)nc(-c3ccc(-c4ccccc4)c4oc5ccccc5c34)n2)cc1 ; c1ccc(-c2nc(-c3cccc(-c4cccc5c(-c6ccccc6)cccc45)c3)nc(-c3cccc4oc5c(-c6ccccc6)cccc5c34)n2)cc1 ; c1ccc(-c2ccc3c(c2)oc2cccc(-c4nc(-c5ccccc5)nc(-c5cccc(-c6cccc7c(-c8ccccc8)cccc67)c5)n4)c23)cc1 ; c1ccc(-c2ccc3oc4cccc(-c5nc(-c6ccccc6)nc(-c6cccc(-c7cccc8c(-c9ccccc9)cccc78)c6)n5)c4c3c2)cc1]

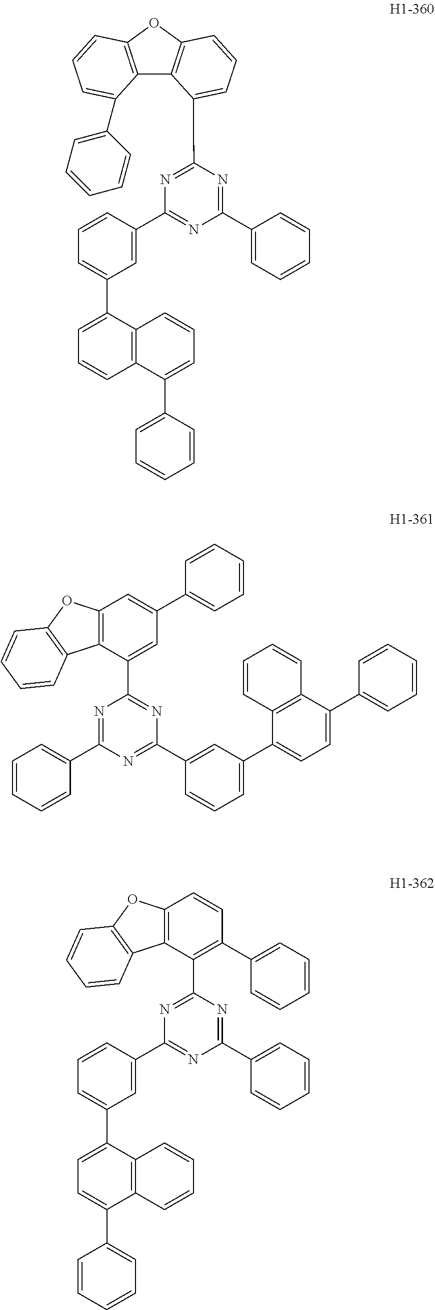 [SMILES: c1ccc(-c2nc(-c3cccc(-c4cccc5c(-c6ccccc6)cccc45)c3)nc(-c3cccc4oc5cccc(-c6ccccc6)c5c34)n2)cc1 ; c1ccc(-c2cc(-c3nc(-c4ccccc4)nc(-c4cccc(-c5ccc(-c6ccccc6)c6ccccc56)c4)n3)c3c(c2)oc2ccccc23)cc1 ; c1ccc(-c2nc(-c3cccc(-c4ccc(-c5ccccc5)c5ccccc45)c3)nc(-c3c(-c4ccccc4)ccc4oc5ccccc5c34)n2)cc1]

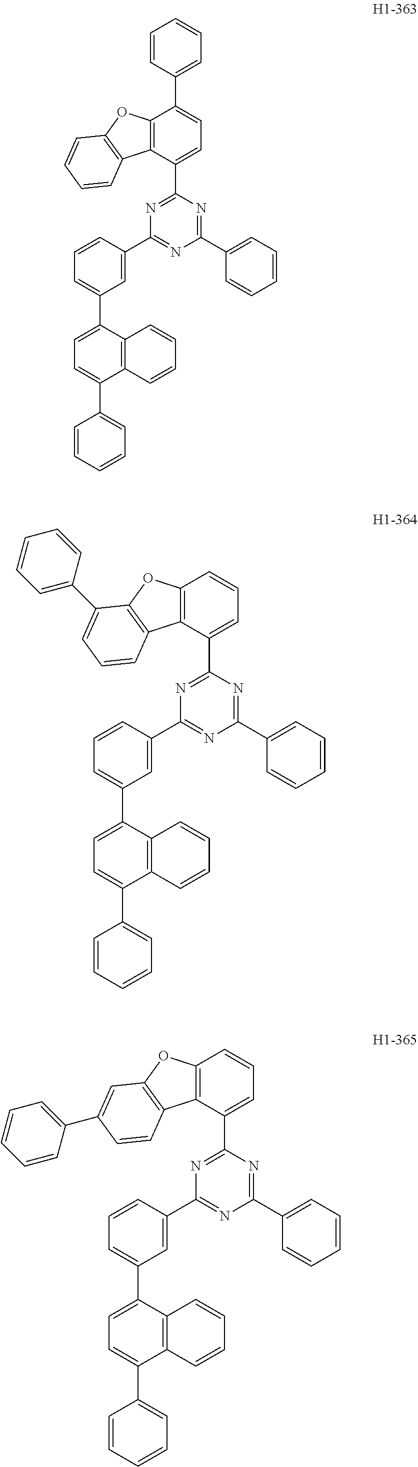 [SMILES: c1ccc(-c2nc(-c3cccc(-c4ccc(-c5ccccc5)c5ccccc45)c3)nc(-c3ccc(-c4ccccc4)c4oc5ccccc5c34)n2)cc1 ; c1ccc(-c2nc(-c3cccc(-c4ccc(-c5ccccc5)c5ccccc45)c3)nc(-c3cccc4oc5c(-c6ccccc6)cccc5c34)n2)cc1 ; c1ccc(-c2ccc3c(c2)oc2cccc(-c4nc(-c5ccccc5)nc(-c5cccc(-c6ccc(-c7ccccc7)c7ccccc67)c5)n4)c23)cc1]

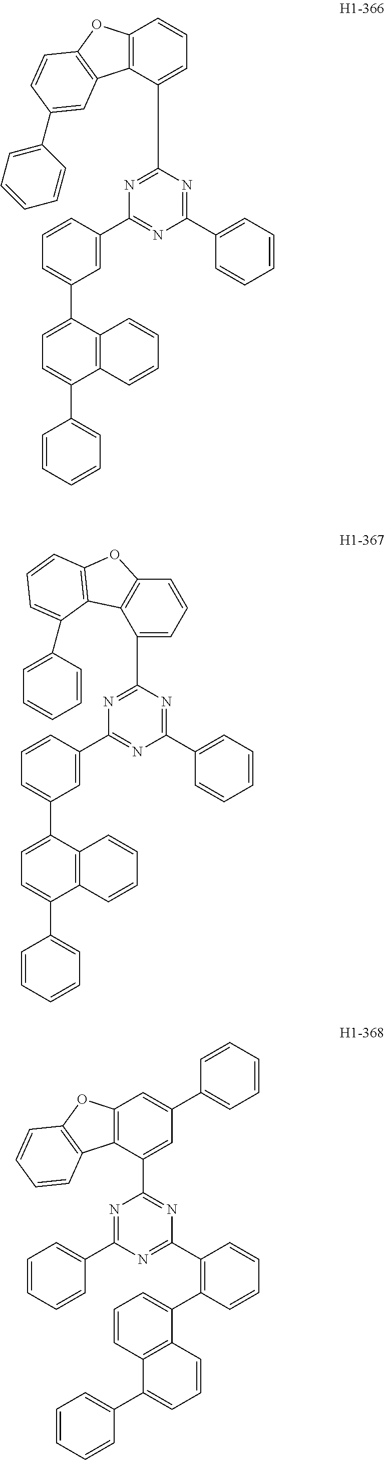 [SMILES: c1ccc(-c2ccc3oc4cccc(-c5nc(-c6ccccc6)nc(-c6cccc(-c7ccc(-c8ccccc8)c8ccccc78)c6)n5)c4c3c2)cc1 ; c1ccc(-c2nc(-c3cccc(-c4ccc(-c5ccccc5)c5ccccc45)c3)nc(-c3cccc4oc5cccc(-c6ccccc6)c5c34)n2)cc1 ; c1ccc(-c2cc(-c3nc(-c4ccccc4)nc(-c4ccccc4-c4cccc5c(-c6ccccc6)cccc45)n3)c3c(c2)oc2ccccc23)cc1]

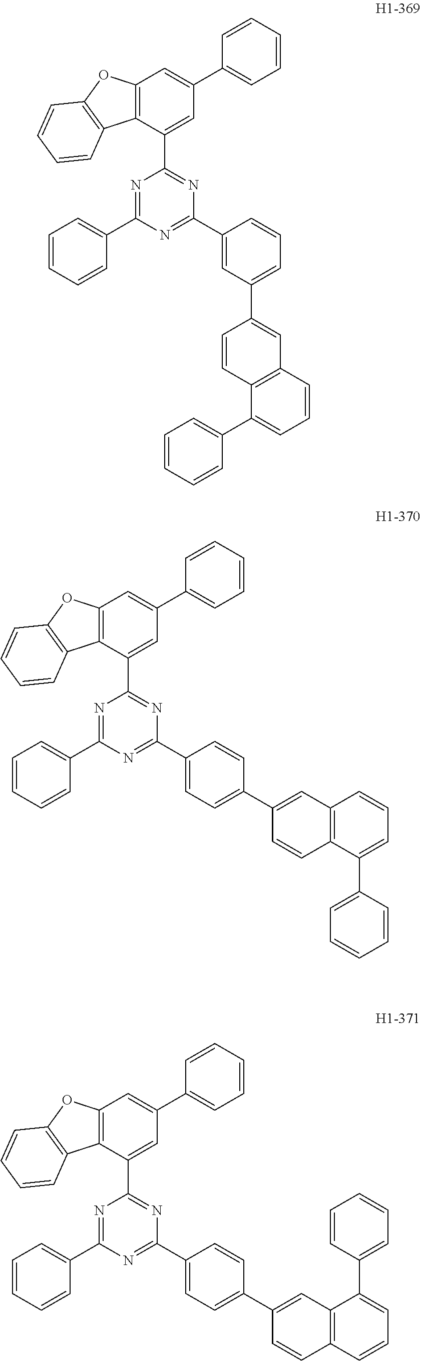 [SMILES: c1ccc(-c2cc(-c3nc(-c4ccccc4)nc(-c4cccc(-c5ccc6c(-c7ccccc7)cccc6c5)c4)n3)c3c(c2)oc2ccccc23)cc1 ; c1ccc(-c2cc(-c3nc(-c4ccccc4)nc(-c4ccc(-c5ccc6c(-c7ccccc7)cccc6c5)cc4)n3)c3c(c2)oc2ccccc23)cc1 ; c1ccc(-c2cc(-c3nc(-c4ccccc4)nc(-c4ccc(-c5ccc6cccc(-c7ccccc7)c6c5)cc4)n3)c3c(c2)oc2ccccc23)cc1]

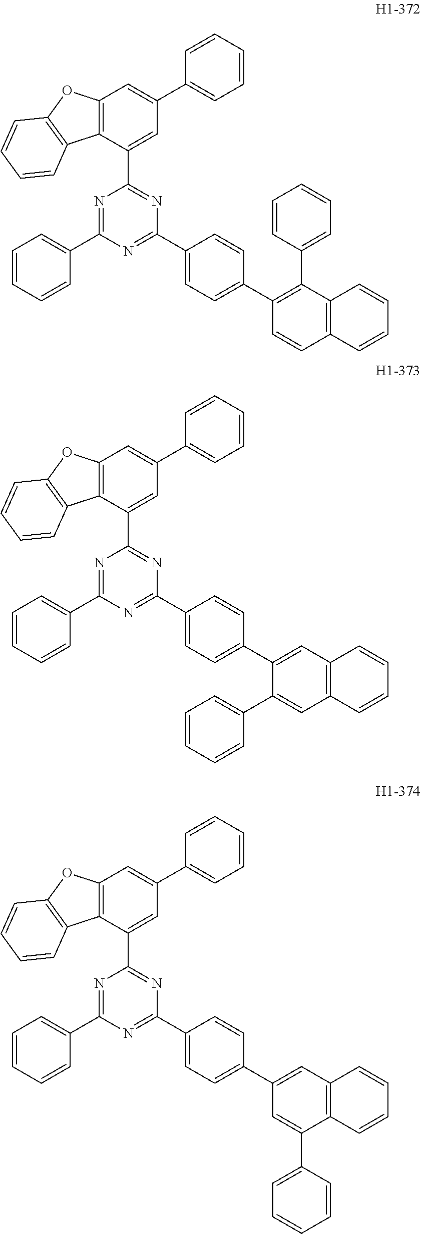 [SMILES: c1ccc(-c2cc(-c3nc(-c4ccccc4)nc(-c4ccc(-c5ccc6ccccc6c5-c5ccccc5)cc4)n3)c3c(c2)oc2ccccc23)cc1 ; c1ccc(-c2cc(-c3nc(-c4ccccc4)nc(-c4ccc(-c5cc6ccccc6cc5-c5ccccc5)cc4)n3)c3c(c2)oc2ccccc23)cc1 ; c1ccc(-c2cc(-c3nc(-c4ccccc4)nc(-c4ccc(-c5cc(-c6ccccc6)c6ccccc6c5)cc4)n3)c3c(c2)oc2ccccc23)cc1]

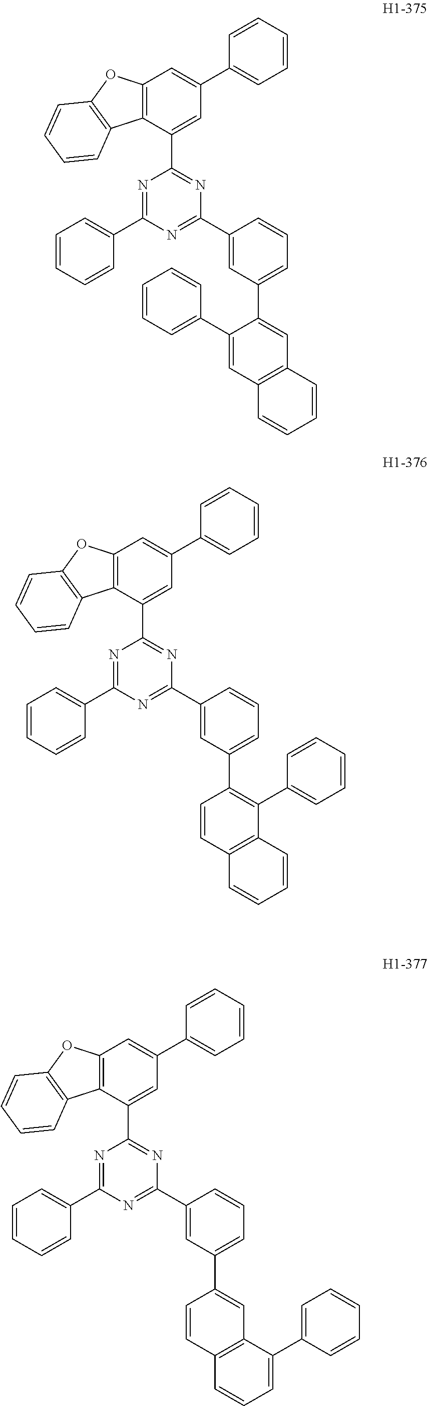 [SMILES: c1ccc(-c2cc(-c3nc(-c4ccccc4)nc(-c4cccc(-c5cc6ccccc6cc5-c5ccccc5)c4)n3)c3c(c2)oc2ccccc23)cc1 ; c1ccc(-c2cc(-c3nc(-c4ccccc4)nc(-c4cccc(-c5ccc6ccccc6c5-c5ccccc5)c4)n3)c3c(c2)oc2ccccc23)cc1 ; c1ccc(-c2cc(-c3nc(-c4ccccc4)nc(-c4cccc(-c5ccc6cccc(-c7ccccc7)c6c5)c4)n3)c3c(c2)oc2ccccc23)cc1]

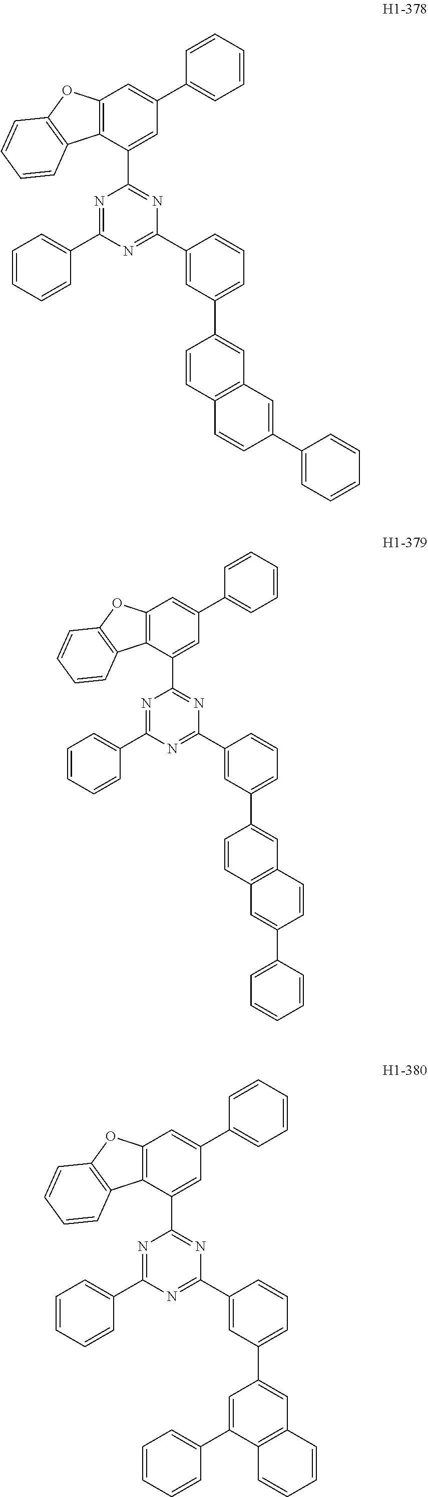 [SMILES: c1ccc(-c2ccc3ccc(-c4cccc(-c5nc(-c6ccccc6)nc(-c6cc(-c7ccccc7)cc7oc8ccccc8c67)n5)c4)cc3c2)cc1 ; c1ccc(-c2ccc3cc(-c4cccc(-c5nc(-c6ccccc6)nc(-c6cc(-c7ccccc7)cc7oc8ccccc8c67)n5)c4)ccc3c2)cc1 ; c1ccc(-c2cc(-c3nc(-c4ccccc4)nc(-c4cccc(-c5cc(-c6ccccc6)c6ccccc6c5)c4)n3)c3c(c2)oc2ccccc23)cc1]

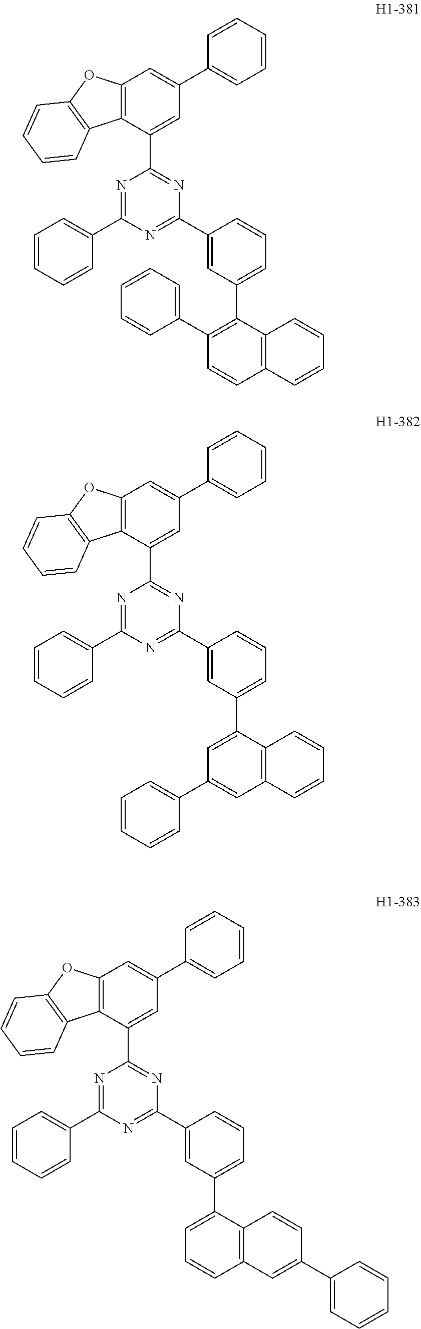

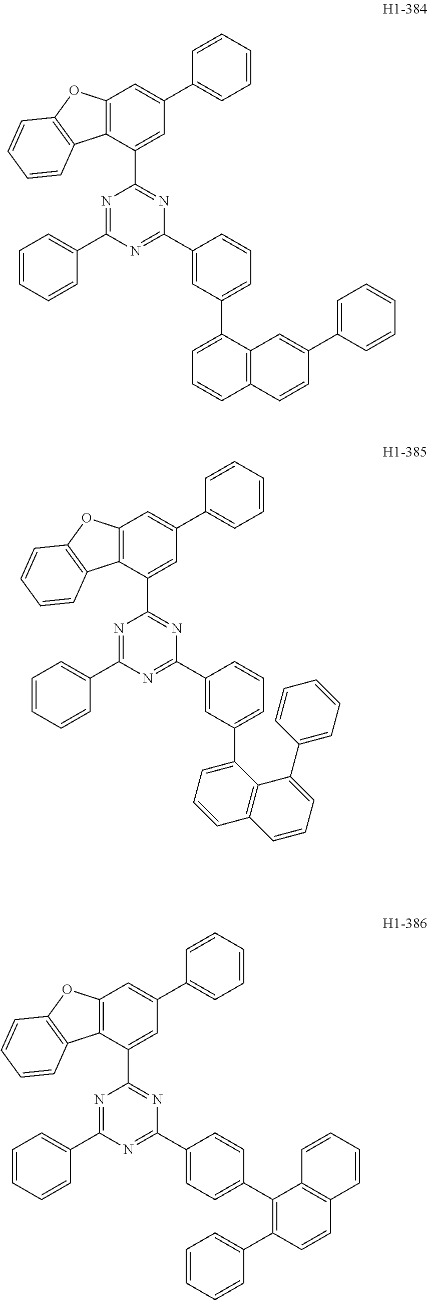 [SMILES: c1ccc(-c2cc(-c3nc(-c4ccccc4)nc(-c4cccc(-c5cccc6ccc(-c7ccccc7)cc56)c4)n3)c3c(c2)oc2ccccc23)cc1 ; c1ccc(-c2cc(-c3nc(-c4ccccc4)nc(-c4cccc(-c5cccc6cccc(-c7ccccc7)c56)c4)n3)c3c(c2)oc2ccccc23)cc1 ; c1ccc(-c2cc(-c3nc(-c4ccccc4)nc(-c4ccc(-c5c(-c6ccccc6)ccc6ccccc56)cc4)n3)c3c(c2)oc2ccccc23)cc1]

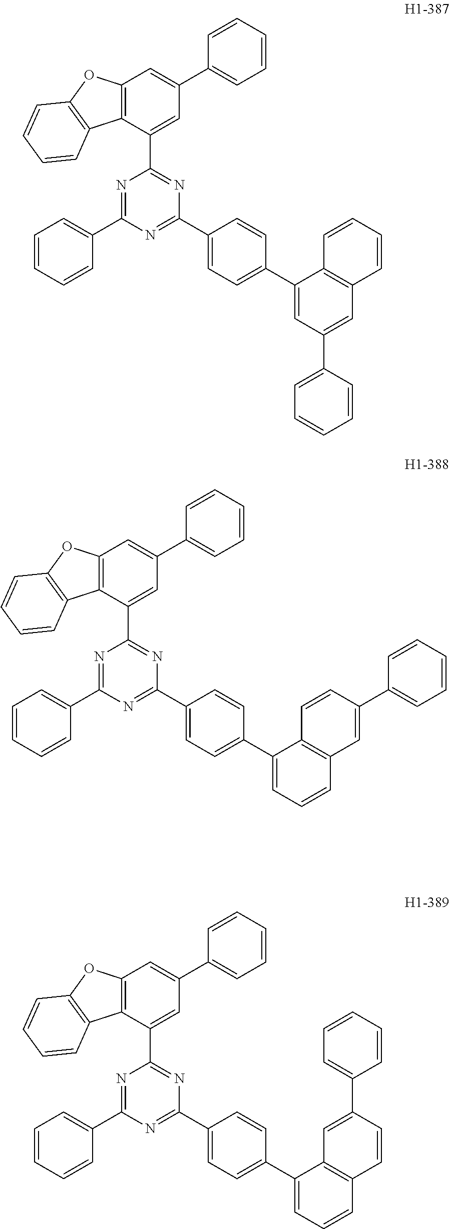 [SMILES: c1ccc(-c2cc(-c3ccc(-c4nc(-c5ccccc5)nc(-c5cc(-c6ccccc6)cc6oc7ccccc7c56)n4)cc3)c3ccccc3c2)cc1 ; c1ccc(-c2ccc3c(-c4ccc(-c5nc(-c6ccccc6)nc(-c6cc(-c7ccccc7)cc7oc8ccccc8c67)n5)cc4)cccc3c2)cc1 ; c1ccc(-c2cc(-c3nc(-c4ccccc4)nc(-c4ccc(-c5cccc6ccc(-c7ccccc7)cc56)cc4)n3)c3c(c2)oc2ccccc23)cc1]

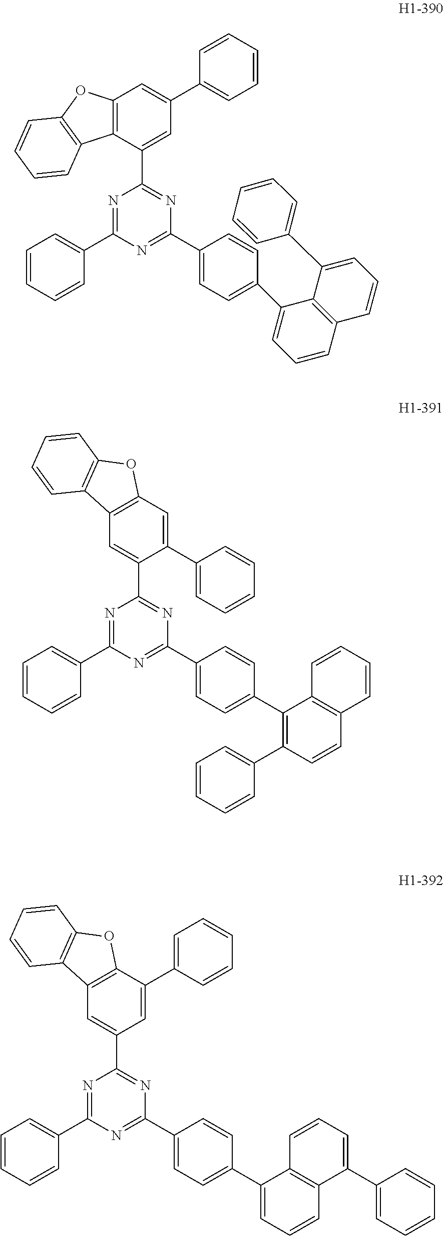 [SMILES: c1ccc(-c2cc(-c3nc(-c4ccccc4)nc(-c4ccc(-c5cccc6cccc(-c7ccccc7)c56)cc4)n3)c3c(c2)oc2ccccc23)cc1 ; c1ccc(-c2nc(-c3ccc(-c4c(-c5ccccc5)ccc5ccccc45)cc3)nc(-c3cc4c(cc3-c3ccccc3)oc3ccccc34)n2)cc1 ; c1ccc(-c2nc(-c3ccc(-c4cccc5c(-c6ccccc6)cccc45)cc3)nc(-c3cc(-c4ccccc4)c4oc5ccccc5c4c3)n2)cc1]

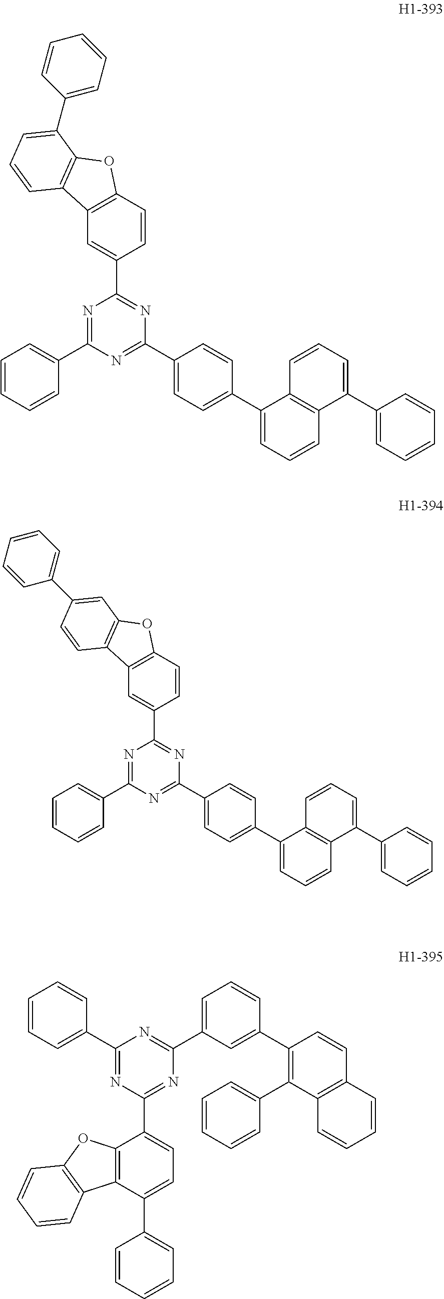 [SMILES: c1ccc(-c2nc(-c3ccc(-c4cccc5c(-c6ccccc6)cccc45)cc3)nc(-c3ccc4oc5c(-c6ccccc6)cccc5c4c3)n2)cc1 ; c1ccc(-c2ccc3c(c2)oc2ccc(-c4nc(-c5ccccc5)nc(-c5ccc(-c6cccc7c(-c8ccccc8)cccc67)cc5)n4)cc23)cc1 ; c1ccc(-c2nc(-c3cccc(-c4ccc5ccccc5c4-c4ccccc4)c3)nc(-c3ccc(-c4ccccc4)c4c3oc3ccccc34)n2)cc1]

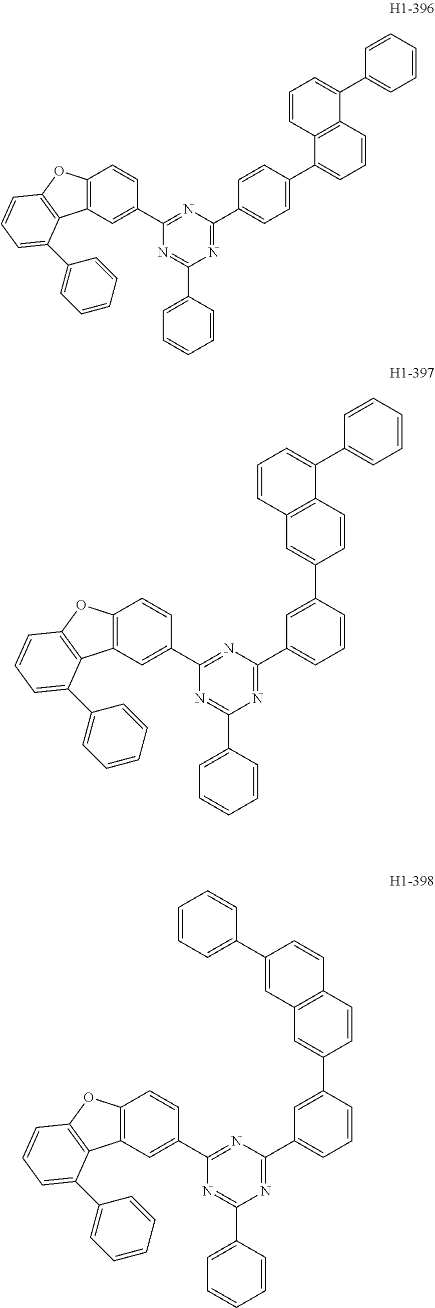 [SMILES: c1ccc(-c2nc(-c3ccc(-c4cccc5c(-c6ccccc6)cccc45)cc3)nc(-c3ccc4oc5cccc(-c6ccccc6)c5c4c3)n2)cc1 ; c1ccc(-c2nc(-c3cccc(-c4ccc5c(-c6ccccc6)cccc5c4)c3)nc(-c3ccc4oc5cccc(-c6ccccc6)c5c4c3)n2)cc1 ; c1ccc(-c2ccc3ccc(-c4cccc(-c5nc(-c6ccccc6)nc(-c6ccc7oc8cccc(-c9ccccc9)c8c7c6)n5)c4)cc3c2)cc1]

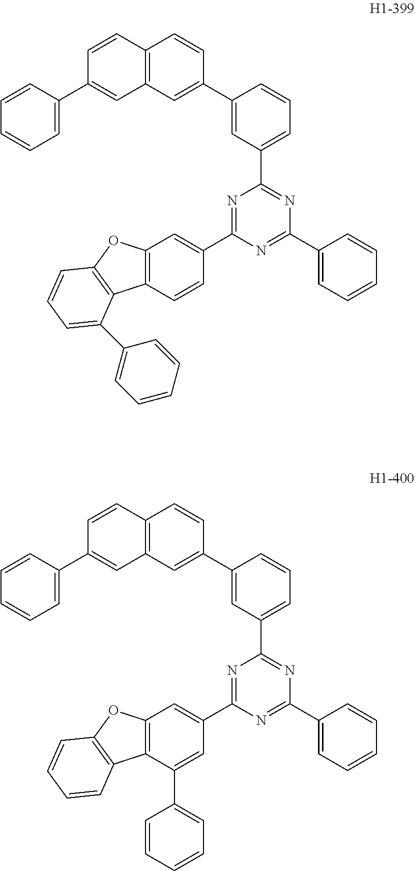 [SMILES: c1ccc(-c2ccc3ccc(-c4cccc(-c5nc(-c6ccccc6)nc(-c6ccc7c(c6)oc6cccc(-c8ccccc8)c67)n5)c4)cc3c2)cc1 ; c1ccc(-c2ccc3ccc(-c4cccc(-c5nc(-c6ccccc6)nc(-c6cc(-c7ccccc7)c7c(c6)oc6ccccc67)n5)c4)cc3c2)cc1]

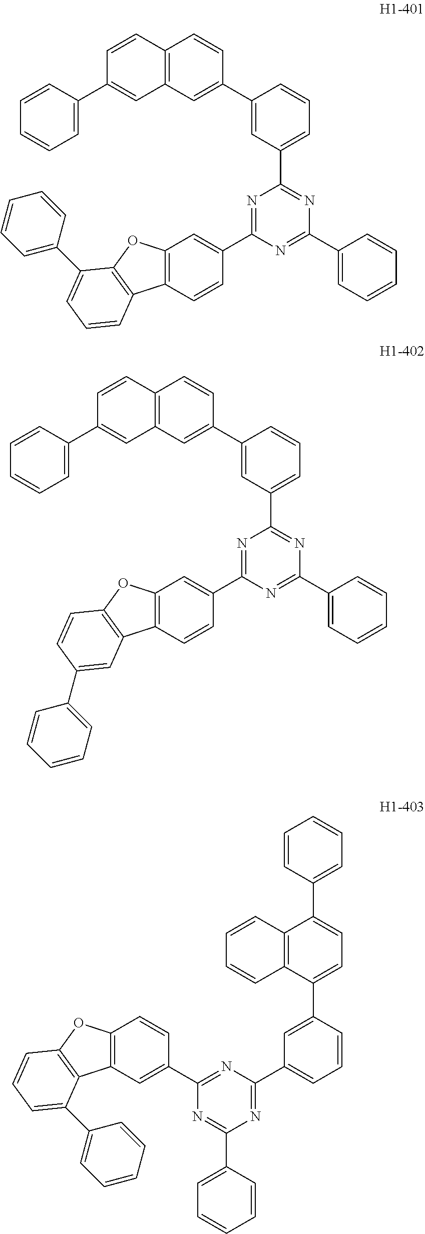 [SMILES: c1ccc(-c2ccc3ccc(-c4cccc(-c5nc(-c6ccccc6)nc(-c6ccc7c(c6)oc6c(-c8ccccc8)cccc67)n5)c4)cc3c2)cc1 ; c1ccc(-c2ccc3ccc(-c4cccc(-c5nc(-c6ccccc6)nc(-c6ccc7c(c6)oc6ccc(-c8ccccc8)cc67)n5)c4)cc3c2)cc1 ; c1ccc(-c2nc(-c3cccc(-c4ccc(-c5ccccc5)c5ccccc45)c3)nc(-c3ccc4oc5cccc(-c6ccccc6)c5c4c3)n2)cc1]

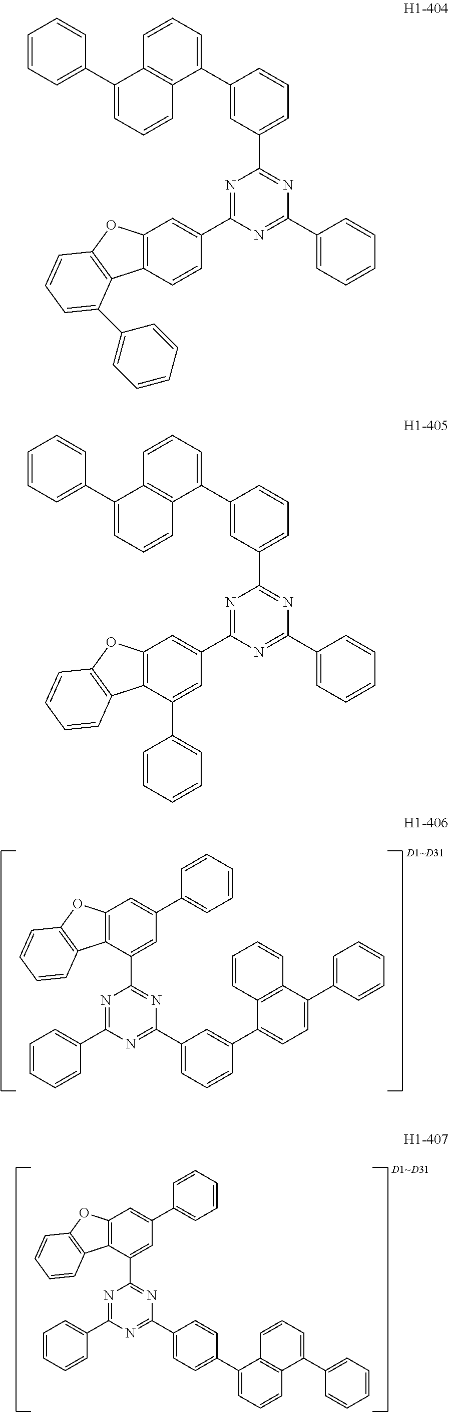

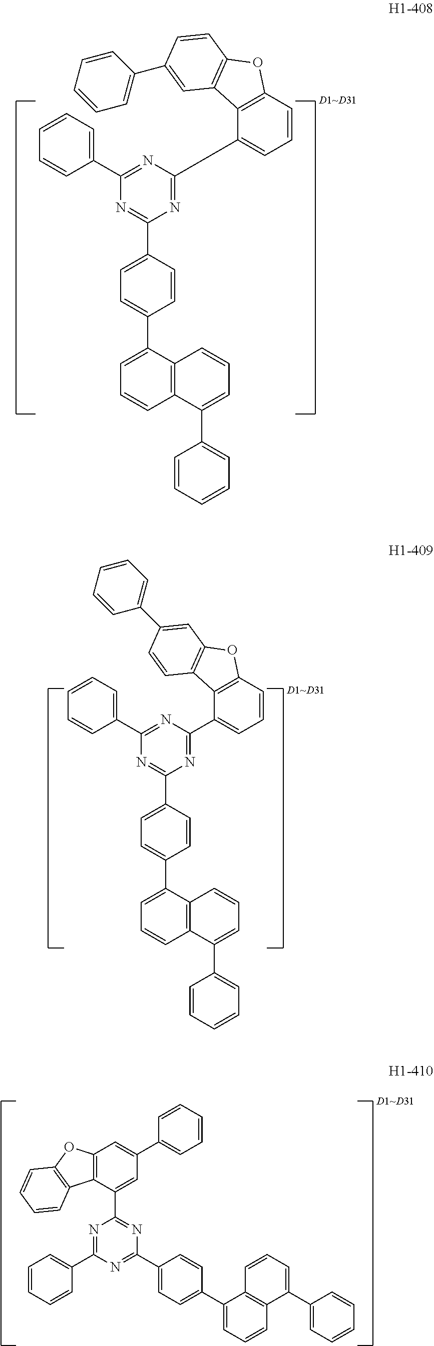

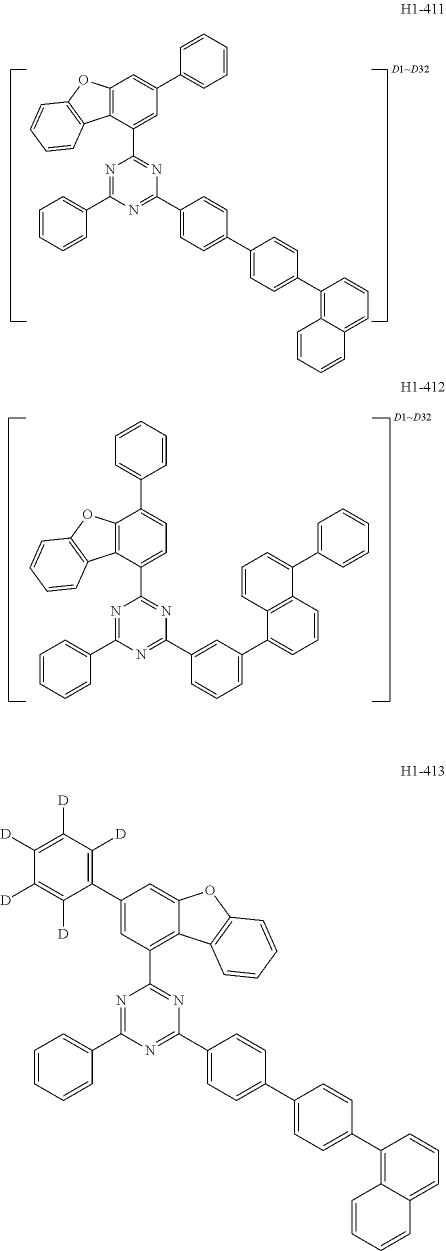

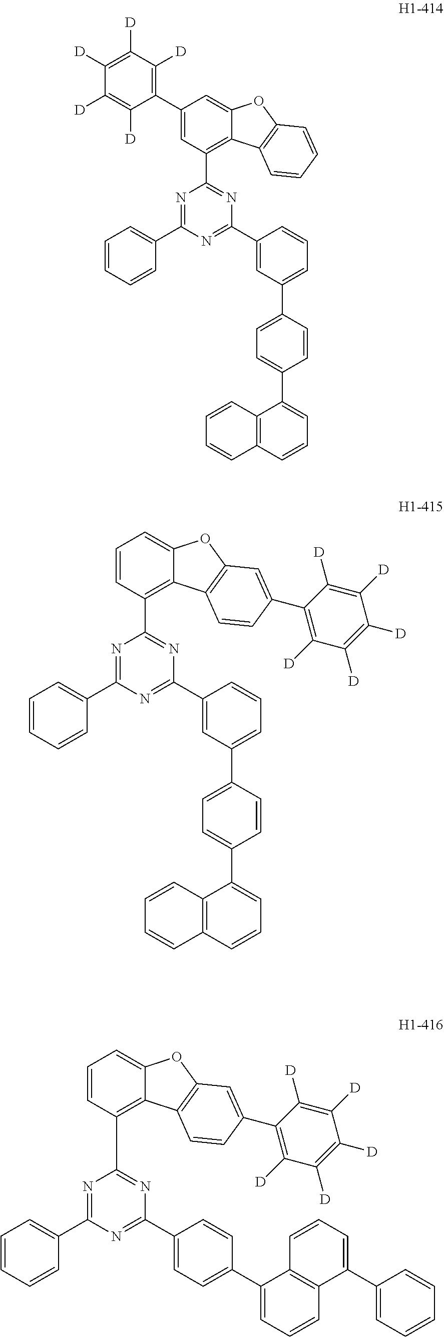 [SMILES: [2H]c1c([2H])c([2H])c(-c2cc(-c3nc(-c4ccccc4)nc(-c4cccc(-c5ccc(-c6cccc7ccccc67)cc5)c4)n3)c3c(c2)oc2ccccc23)c([2H])c1[2H] ; [2H]c1c([2H])c([2H])c(-c2ccc3c(c2)oc2cccc(-c4nc(-c5ccccc5)nc(-c5cccc(-c6ccc(-c7cccc8ccccc78)cc6)c5)n4)c23)c([2H])c1[2H] ; [2H]c1c([2H])c([2H])c(-c2ccc3c(c2)oc2cccc(-c4nc(-c5ccccc5)nc(-c5ccc(-c6cccc7c(-c8ccccc8)cccc67)cc5)n4)c23)c([2H])c1[2H]]

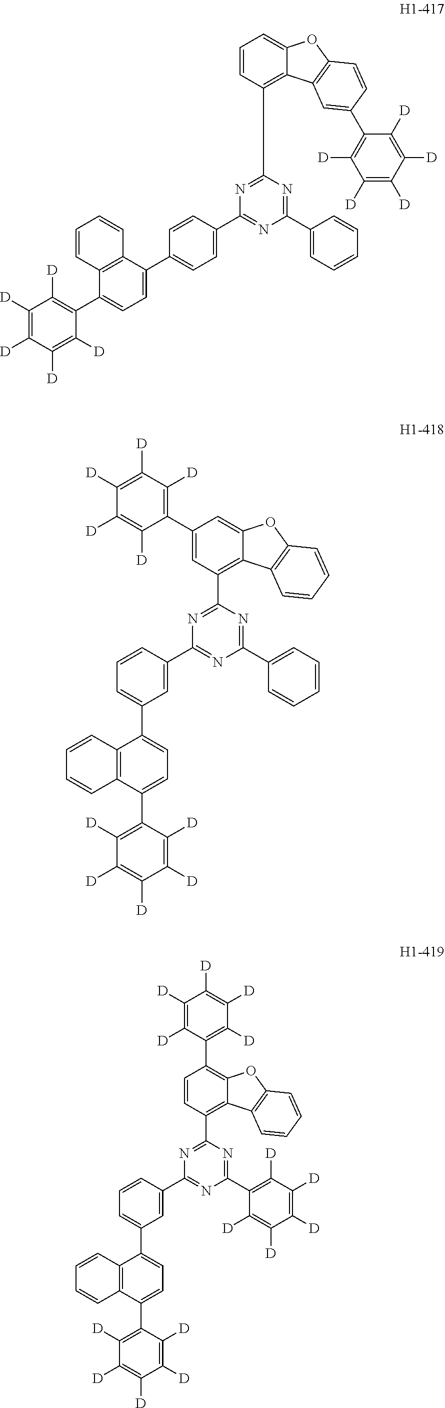 [SMILES: [2H]c1c([2H])c([2H])c(-c2ccc3oc4cccc(-c5nc(-c6ccccc6)nc(-c6ccc(-c7ccc(-c8c([2H])c([2H])c([2H])c([2H])c8[2H])c8ccccc78)cc6)n5)c4c3c2)c([2H])c1[2H] ; [2H]c1c([2H])c([2H])c(-c2cc(-c3nc(-c4ccccc4)nc(-c4cccc(-c5ccc(-c6c([2H])c([2H])c([2H])c([2H])c6[2H])c6ccccc56)c4)n3)c3c(c2)oc2ccccc23)c([2H])c1[2H] ; [2H]c1c([2H])c([2H])c(-c2nc(-c3cccc(-c4ccc(-c5c([2H])c([2H])c([2H])c([2H])c5[2H])c5ccccc45)c3)nc(-c3ccc(-c4c([2H])c([2H])c([2H])c([2H])c4[2H])c4oc5ccccc5c34)n2)c([2H])c1[2H]]

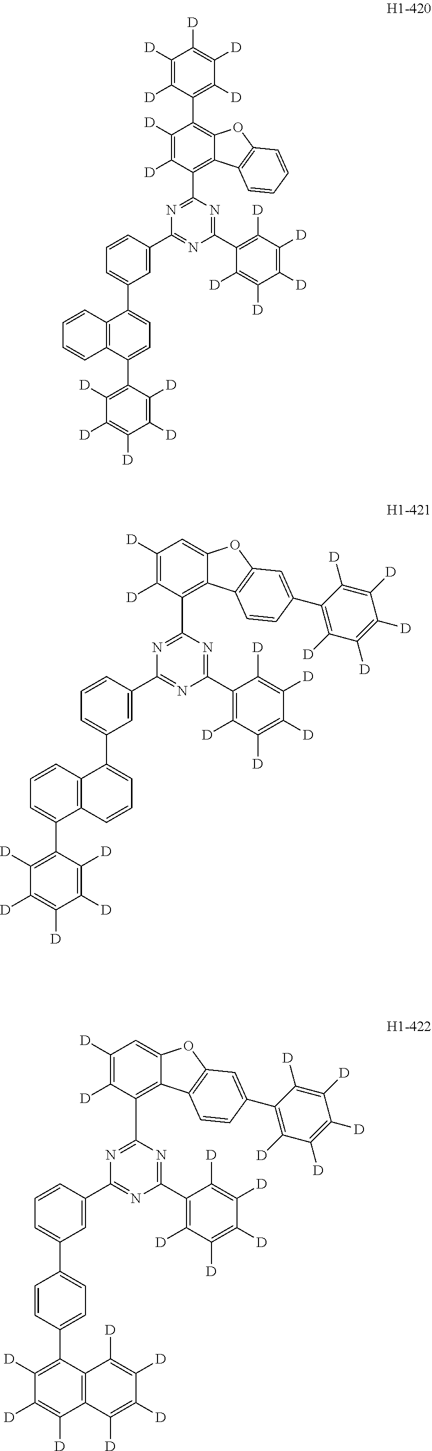

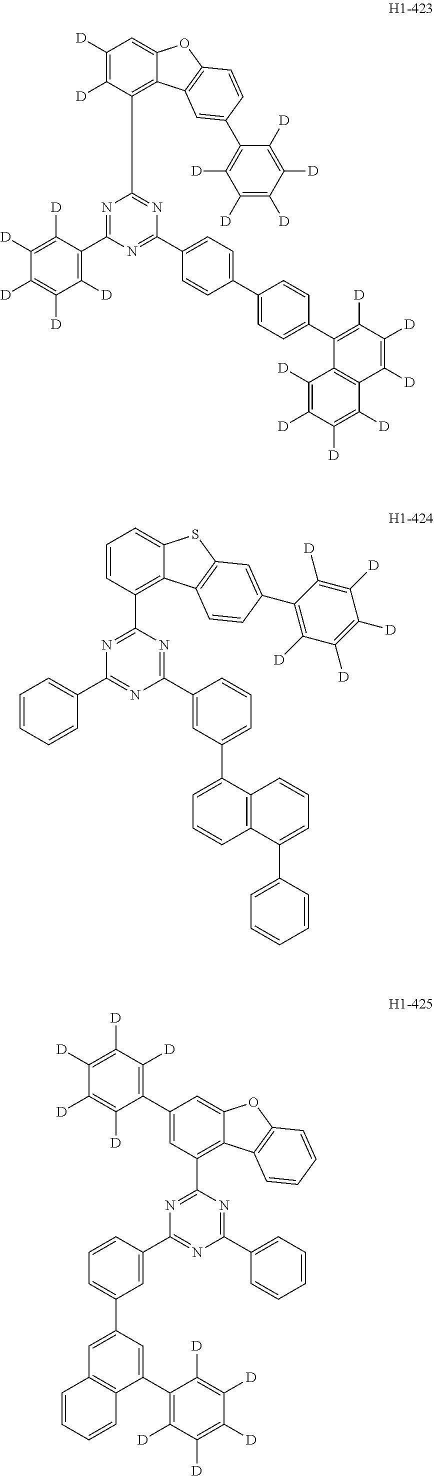 [SMILES: [2H]c1cc2oc3ccc(-c4c([2H])c([2H])c([2H])c([2H])c4[2H])cc3c2c(-c2nc(-c3ccc(-c4ccc(-c5c([2H])c([2H])c([2H])c6c([2H])c([2H])c([2H])c([2H])c56)cc4)cc3)nc(-c3c([2H])c([2H])c([2H])c([2H])c3[2H])n2)c1[2H] ; [2H]c1c([2H])c([2H])c(-c2ccc3c(c2)sc2cccc(-c4nc(-c5ccccc5)nc(-c5cccc(-c6cccc7c(-c8ccccc8)cccc67)c5)n4)c23)c([2H])c1[2H] ; [2H]c1c([2H])c([2H])c(-c2cc(-c3nc(-c4ccccc4)nc(-c4cccc(-c5cc(-c6c([2H])c([2H])c([2H])c([2H])c6[2H])c6ccccc6c5)c4)n3)c3c(c2)oc2ccccc23)c([2H])c1[2H]]

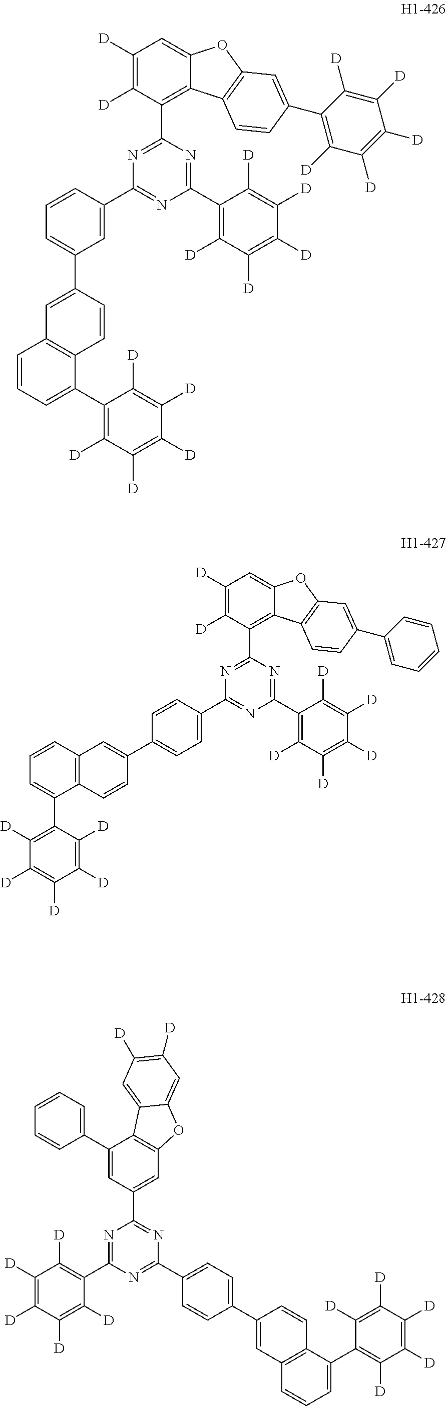 [SMILES: [2H]c1cc2oc3cc(-c4c([2H])c([2H])c([2H])c([2H])c4[2H])ccc3c2c(-c2nc(-c3cccc(-c4ccc5c(-c6c([2H])c([2H])c([2H])c([2H])c6[2H])cccc5c4)c3)nc(-c3c([2H])c([2H])c([2H])c([2H])c3[2H])n2)c1[2H] ; [2H]c1cc2oc3cc(-c4ccccc4)ccc3c2c(-c2nc(-c3ccc(-c4ccc5c(-c6c([2H])c([2H])c([2H])c([2H])c6[2H])cccc5c4)cc3)nc(-c3c([2H])c([2H])c([2H])c([2H])c3[2H])n2)c1[2H] ; [2H]c1cc2oc3cc(-c4nc(-c5ccc(-c6ccc7c(-c8c([2H])c([2H])c([2H])c([2H])c8[2H])cccc7c6)cc5)nc(-c5c([2H])c([2H])c([2H])c([2H])c5[2H])n4)cc(-c4ccccc4)c3c2cc1[2H]]

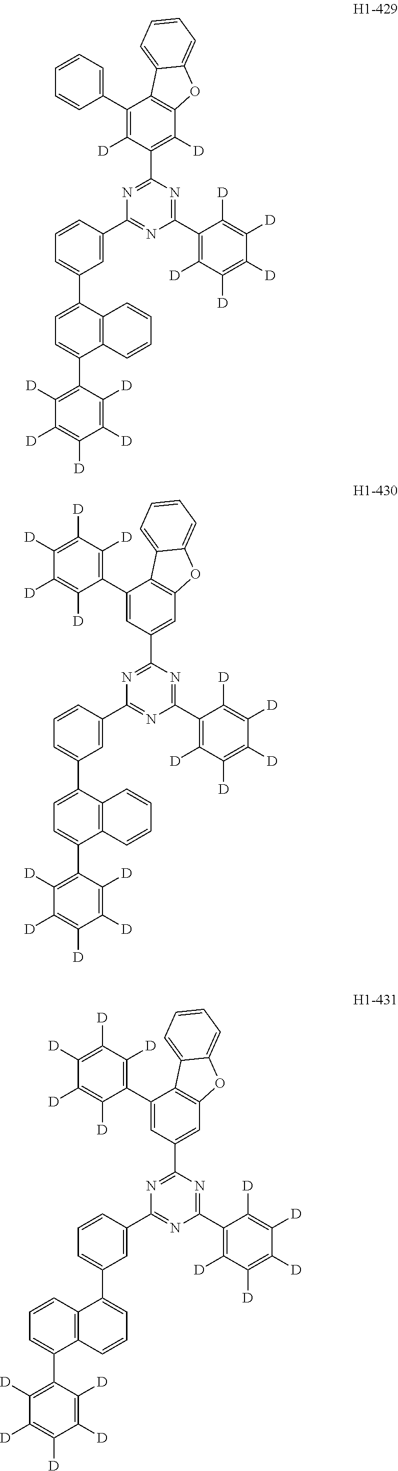

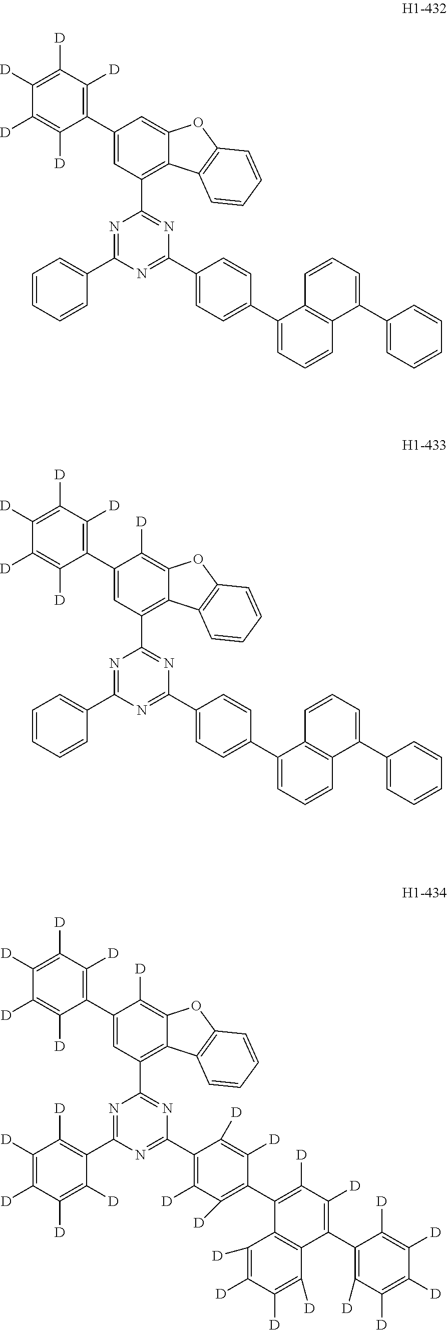 [SMILES: [2H]c1c([2H])c([2H])c(-c2cc(-c3nc(-c4ccccc4)nc(-c4ccc(-c5cccc6c(-c7ccccc7)cccc56)cc4)n3)c3c(c2)oc2ccccc23)c([2H])c1[2H] ; [2H]c1c([2H])c([2H])c(-c2cc(-c3nc(-c4ccccc4)nc(-c4ccc(-c5cccc6c(-c7ccccc7)cccc56)cc4)n3)c3c(oc4ccccc43)c2[2H])c([2H])c1[2H] ; [2H]c1c([2H])c([2H])c(-c2nc(-c3c([2H])c([2H])c(-c4c([2H])c([2H])c(-c5c([2H])c([2H])c([2H])c([2H])c5[2H])c5c([2H])c([2H])c([2H])c([2H])c45)c([2H])c3[2H])nc(-c3cc(-c4c([2H])c([2H])c([2H])c([2H])c4[2H])c([2H])c4oc5ccccc5c34)n2)c([2H])c1[2H]]

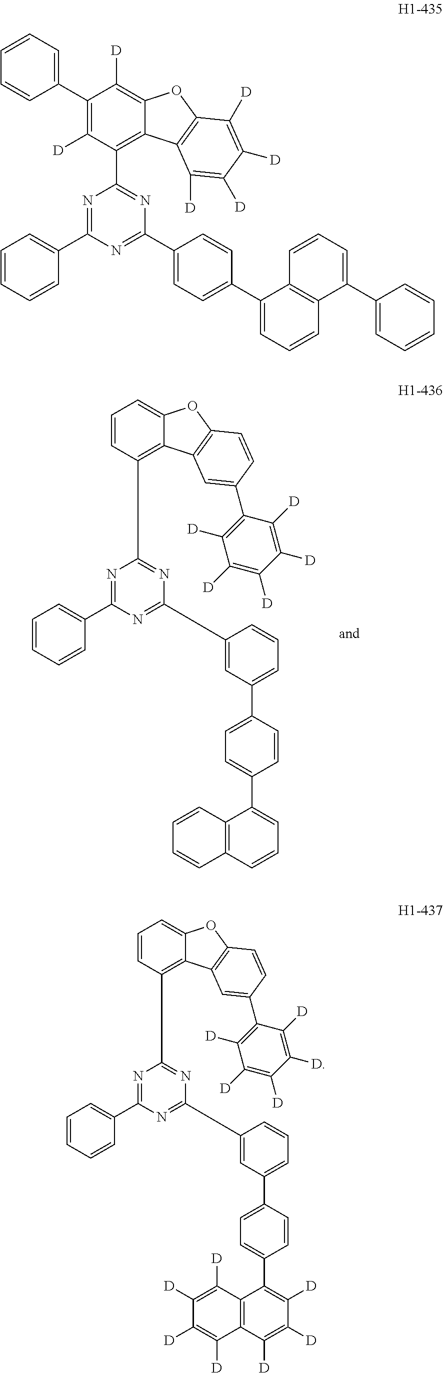

11. The plurality of host materials according to claim 1, wherein the compound represented by Formula 2 is selected from the following compounds:

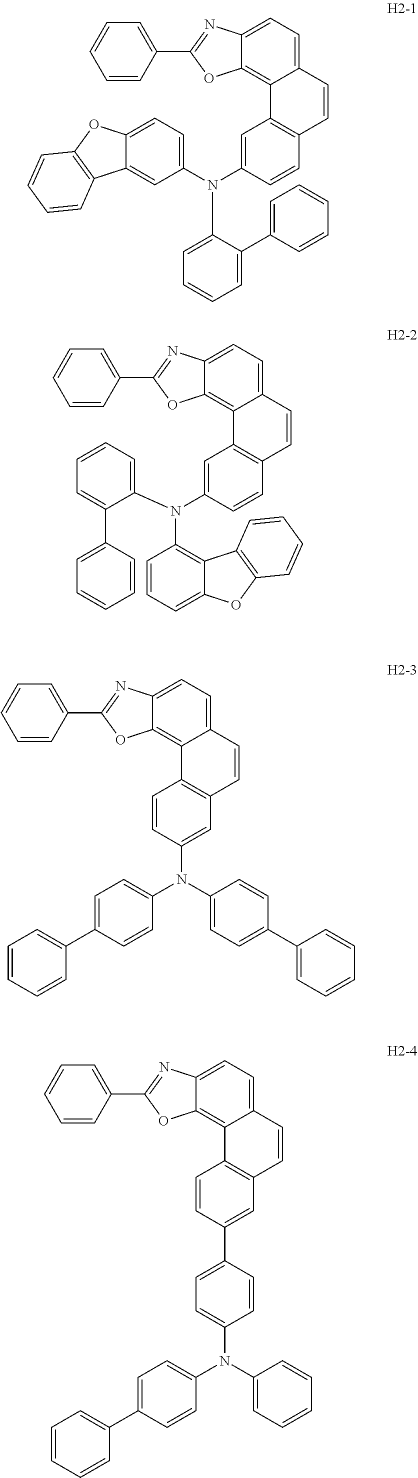 [SMILES: c1ccc(-c2nc3ccc4ccc5ccc(N(c6ccc7oc8ccccc8c7c6)c6ccccc6-c6ccccc6)cc5c4c3o2)cc1 ; c1ccc(-c2nc3ccc4ccc5ccc(N(c6ccccc6-c6ccccc6)c6cccc7oc8ccccc8c67)cc5c4c3o2)cc1 ; c1ccc(-c2ccc(N(c3ccc(-c4ccccc4)cc3)c3ccc4c(ccc5ccc6nc(-c7ccccc7)oc6c54)c3)cc2)cc1 ; c1ccc(-c2ccc(N(c3ccccc3)c3ccc(-c4ccc5c(ccc6ccc7nc(-c8ccccc8)oc7c65)c4)cc3)cc2)cc1]

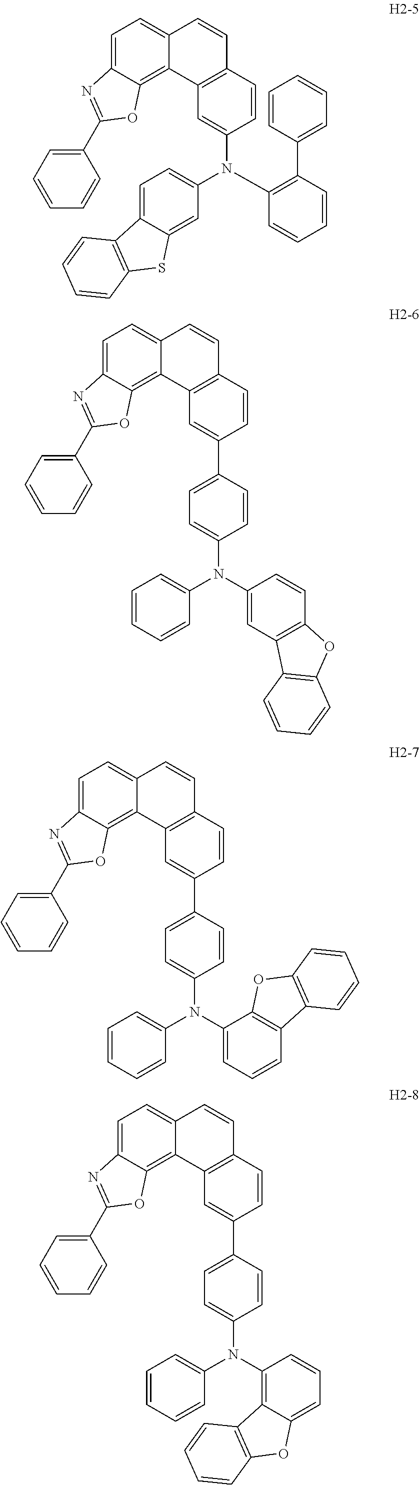

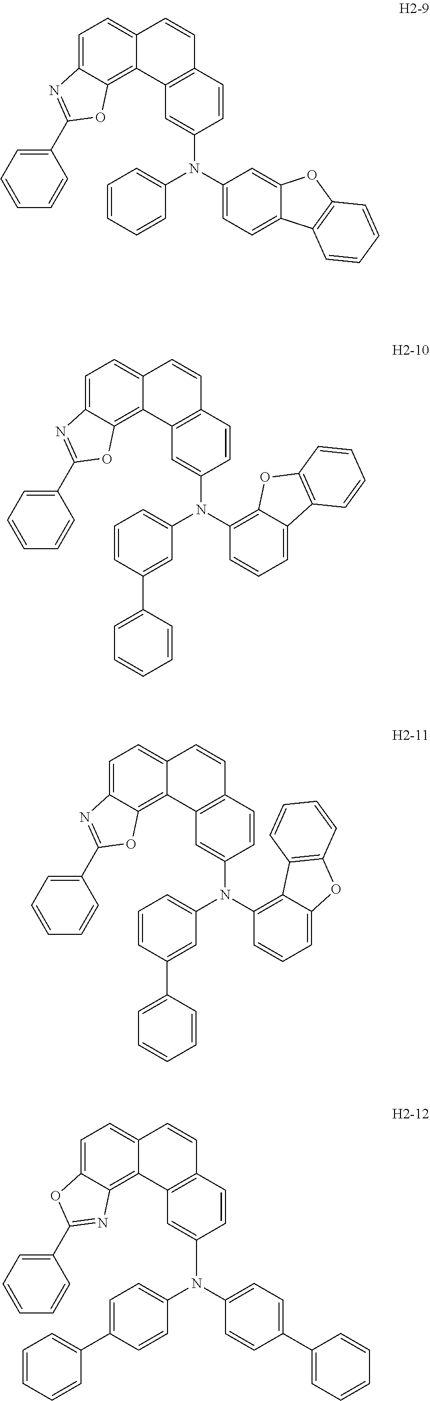 [SMILES: c1ccc(-c2nc3ccc4ccc5ccc(N(c6ccccc6)c6ccc7c(c6)oc6ccccc67)cc5c4c3o2)cc1 ; c1ccc(-c2cccc(N(c3ccc4ccc5ccc6nc(-c7ccccc7)oc6c5c4c3)c3cccc4c3oc3ccccc34)c2)cc1 ; c1ccc(-c2cccc(N(c3ccc4ccc5ccc6nc(-c7ccccc7)oc6c5c4c3)c3cccc4oc5ccccc5c34)c2)cc1 ; c1ccc(-c2ccc(N(c3ccc(-c4ccccc4)cc3)c3ccc4ccc5ccc6oc(-c7ccccc7)nc6c5c4c3)cc2)cc1]

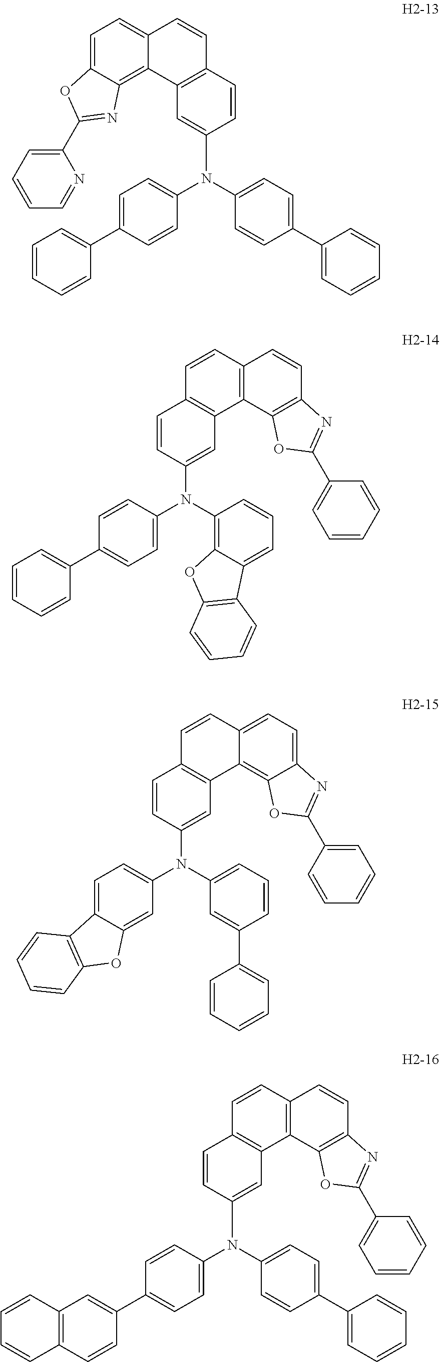

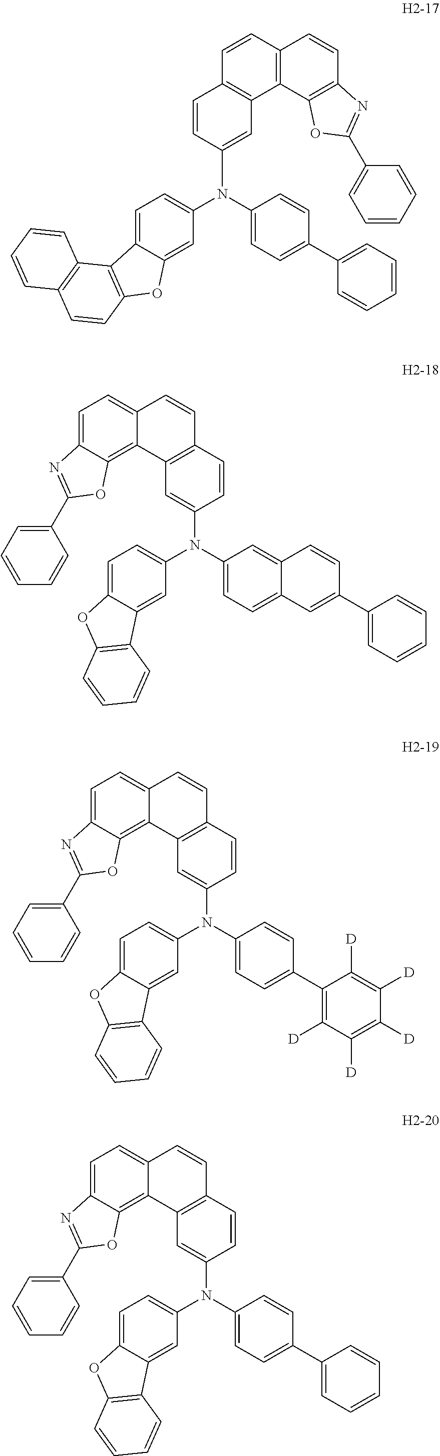

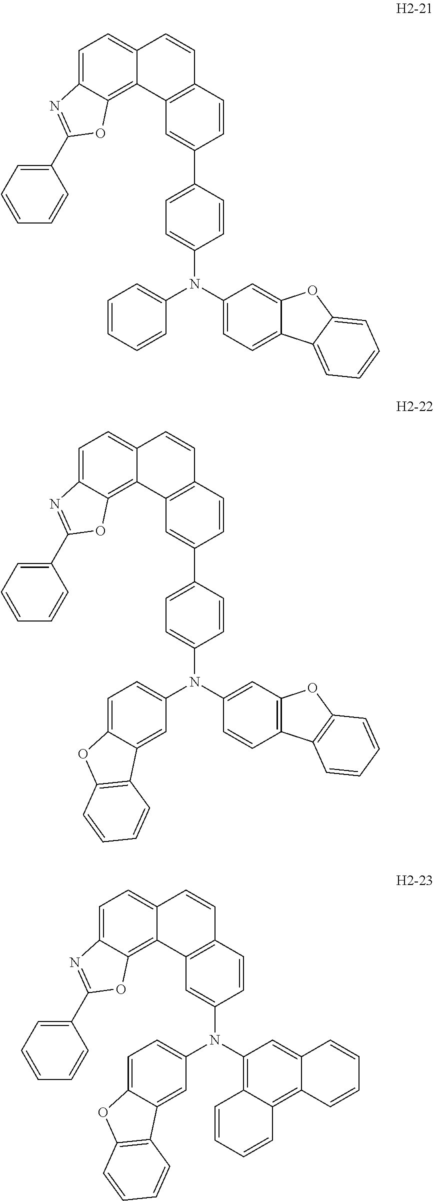 [SMILES: c1ccc(-c2nc3ccc4ccc5ccc(-c6ccc(N(c7ccccc7)c7ccc8c(c7)oc7ccccc78)cc6)cc5c4c3o2)cc1 ; c1ccc(-c2nc3ccc4ccc5ccc(-c6ccc(N(c7ccc8c(c7)oc7ccccc78)c7ccc8oc9ccccc9c8c7)cc6)cc5c4c3o2)cc1 ; c1ccc(-c2nc3ccc4ccc5ccc(N(c6ccc7oc8ccccc8c7c6)c6cc7ccccc7c7ccccc67)cc5c4c3o2)cc1]

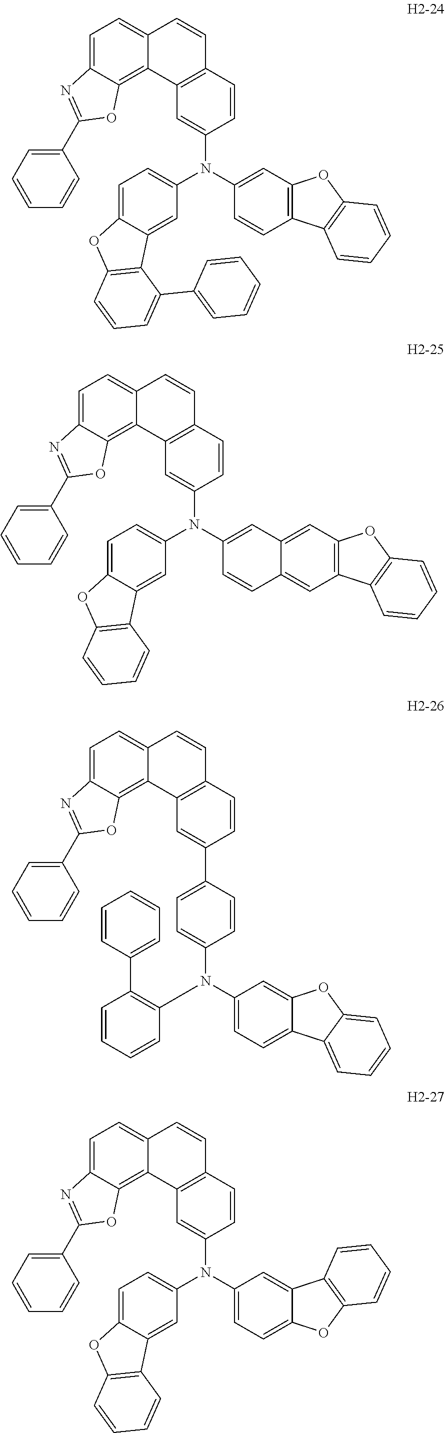

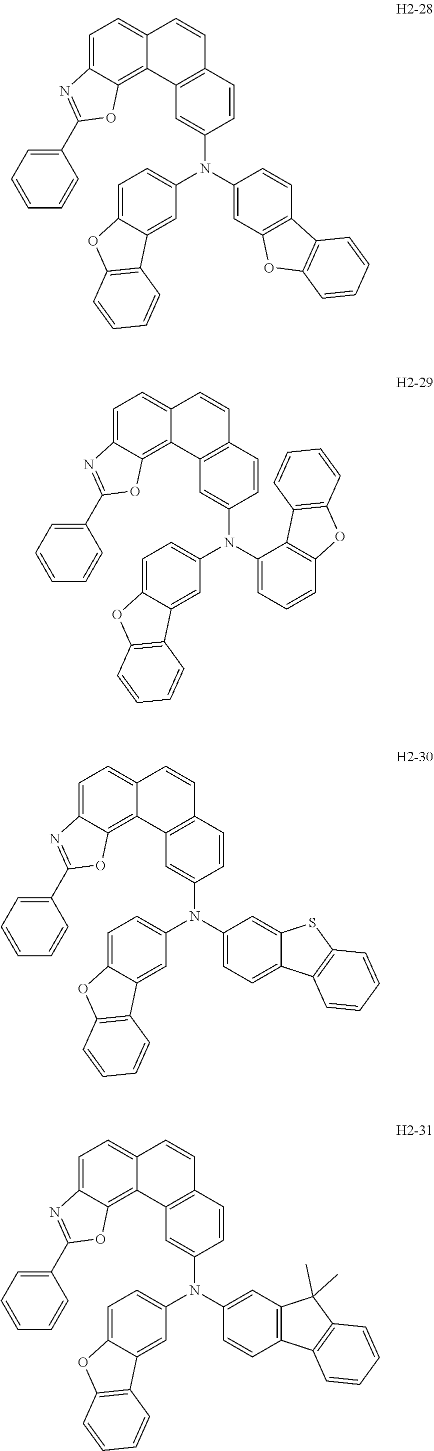 [SMILES: c1ccc(-c2nc3ccc4ccc5ccc(N(c6ccc7c(c6)oc6ccccc67)c6ccc7oc8ccccc8c7c6)cc5c4c3o2)cc1 ; c1ccc(-c2nc3ccc4ccc5ccc(N(c6ccc7oc8ccccc8c7c6)c6cccc7oc8ccccc8c67)cc5c4c3o2)cc1 ; c1ccc(-c2nc3ccc4ccc5ccc(N(c6ccc7c(c6)sc6ccccc67)c6ccc7oc8ccccc8c7c6)cc5c4c3o2)cc1 ; CC1(C)c2ccccc2-c2ccc(N(c3ccc4oc5ccccc5c4c3)c3ccc4ccc5ccc6nc(-c7ccccc7)oc6c5c4c3)cc21]

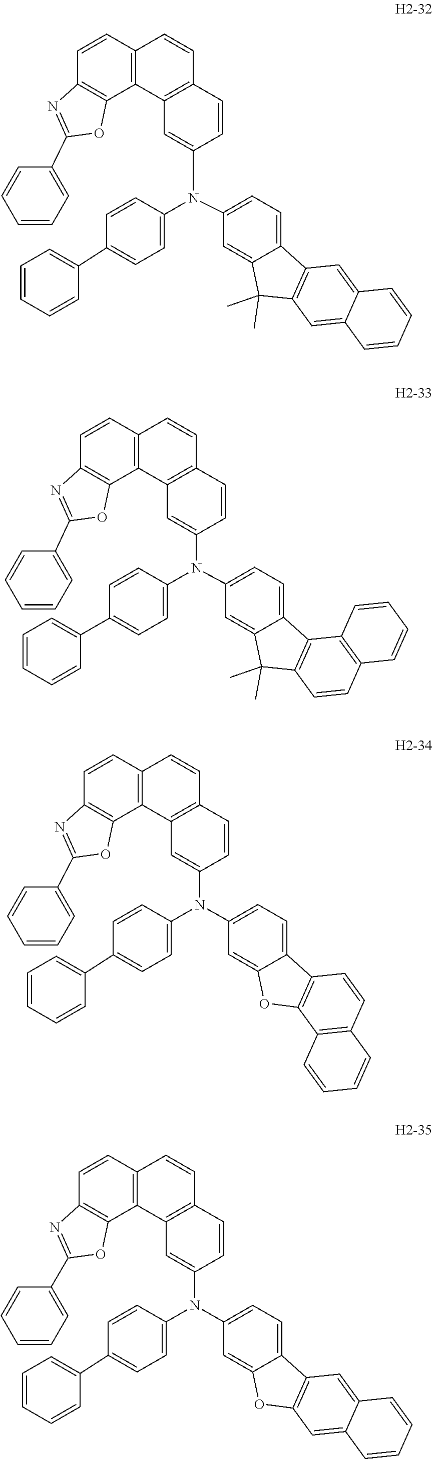 [SMILES: CC1(C)c2cc(N(c3ccc(-c4ccccc4)cc3)c3ccc4ccc5ccc6nc(-c7ccccc7)oc6c5c4c3)ccc2-c2cc3ccccc3cc21 ; CC1(C)c2cc(N(c3ccc(-c4ccccc4)cc3)c3ccc4ccc5ccc6nc(-c7ccccc7)oc6c5c4c3)ccc2-c2c1ccc1ccccc21 ; c1ccc(-c2ccc(N(c3ccc4c(c3)oc3c5ccccc5ccc43)c3ccc4ccc5ccc6nc(-c7ccccc7)oc6c5c4c3)cc2)cc1 ; c1ccc(-c2ccc(N(c3ccc4c(c3)oc3cc5ccccc5cc34)c3ccc4ccc5ccc6nc(-c7ccccc7)oc6c5c4c3)cc2)cc1]

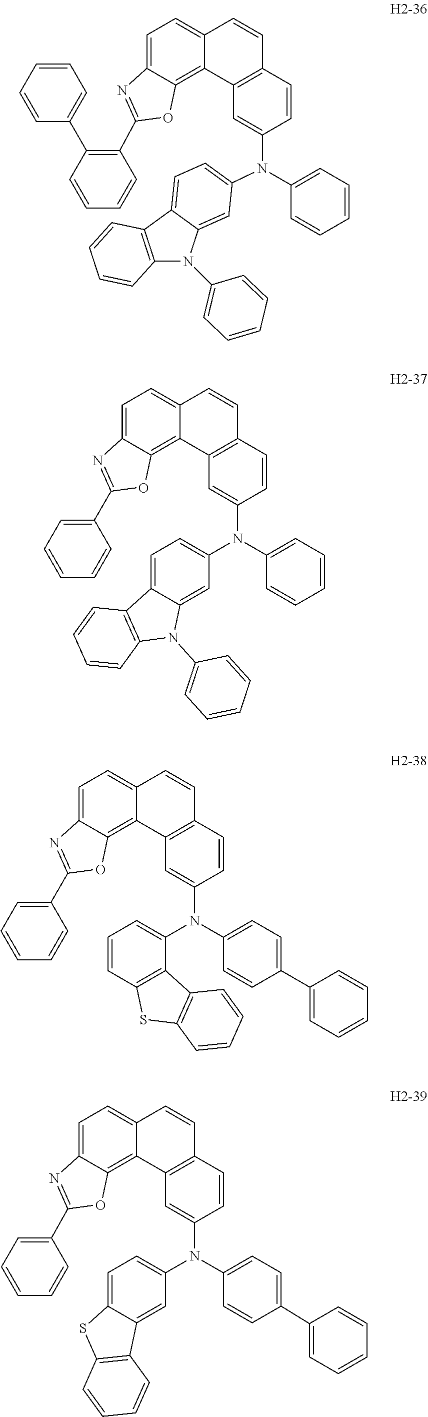 [SMILES: c1ccc(-c2ccccc2-c2nc3ccc4ccc5ccc(N(c6ccccc6)c6ccc7c8ccccc8n(-c8ccccc8)c7c6)cc5c4c3o2)cc1 ; c1ccc(-c2nc3ccc4ccc5ccc(N(c6ccccc6)c6ccc7c8ccccc8n(-c8ccccc8)c7c6)cc5c4c3o2)cc1 ; c1ccc(-c2ccc(N(c3ccc4ccc5ccc6nc(-c7ccccc7)oc6c5c4c3)c3cccc4sc5ccccc5c34)cc2)cc1 ; c1ccc(-c2ccc(N(c3ccc4sc5ccccc5c4c3)c3ccc4ccc5ccc6nc(-c7ccccc7)oc6c5c4c3)cc2)cc1]

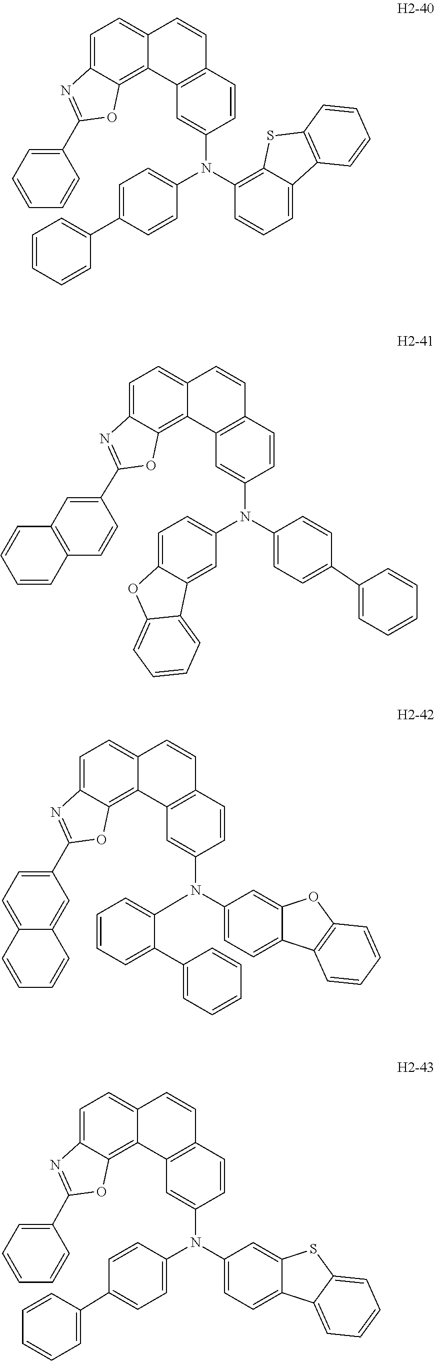 [SMILES: c1ccc(-c2ccc(N(c3ccc4ccc5ccc6nc(-c7ccccc7)oc6c5c4c3)c3cccc4c3sc3ccccc34)cc2)cc1 ; c1ccc(-c2ccc(N(c3ccc4oc5ccccc5c4c3)c3ccc4ccc5ccc6nc(-c7ccc8ccccc8c7)oc6c5c4c3)cc2)cc1 ; c1ccc(-c2ccccc2N(c2ccc3c(c2)oc2ccccc23)c2ccc3ccc4ccc5nc(-c6ccc7ccccc7c6)oc5c4c3c2)cc1 ; c1ccc(-c2ccc(N(c3ccc4c(c3)sc3ccccc34)c3ccc4ccc5ccc6nc(-c7ccccc7)oc6c5c4c3)cc2)cc1]

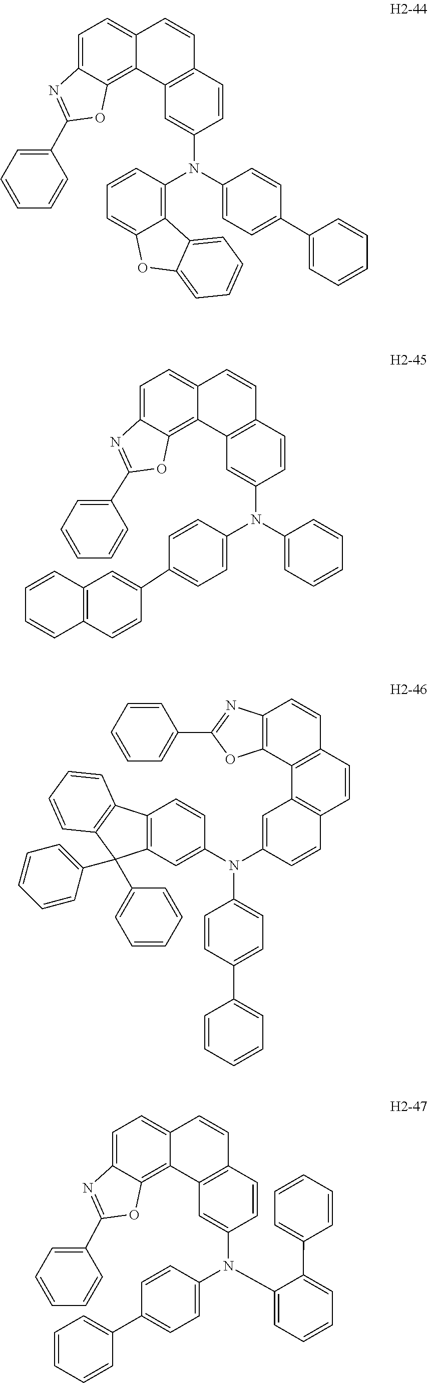 [SMILES: c1ccc(-c2ccc(N(c3ccc4ccc5ccc6nc(-c7ccccc7)oc6c5c4c3)c3cccc4oc5ccccc5c34)cc2)cc1 ; c1ccc(-c2nc3ccc4ccc5ccc(N(c6ccccc6)c6ccc(-c7ccc8ccccc8c7)cc6)cc5c4c3o2)cc1 ; c1ccc(-c2ccc(N(c3ccc4c(c3)C(c3ccccc3)(c3ccccc3)c3ccccc3-4)c3ccc4ccc5ccc6nc(-c7ccccc7)oc6c5c4c3)cc2)cc1 ; c1ccc(-c2ccc(N(c3ccc4ccc5ccc6nc(-c7ccccc7)oc6c5c4c3)c3ccccc3-c3ccccc3)cc2)cc1]

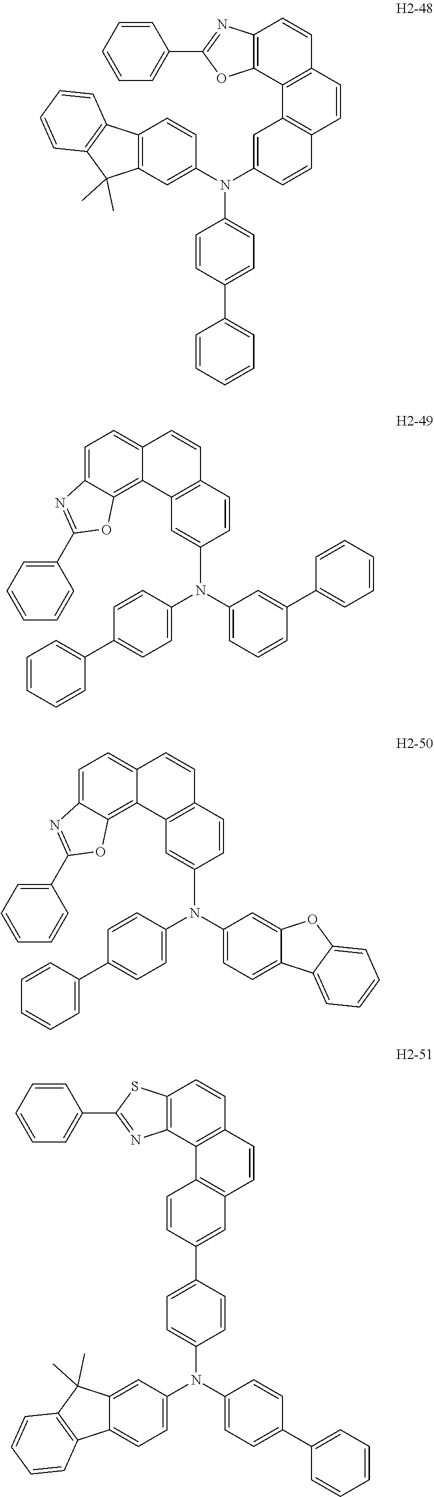 [SMILES: CC1(C)c2ccccc2-c2ccc(N(c3ccc(-c4ccccc4)cc3)c3ccc4ccc5ccc6nc(-c7ccccc7)oc6c5c4c3)cc21 ; c1ccc(-c2ccc(N(c3cccc(-c4ccccc4)c3)c3ccc4ccc5ccc6nc(-c7ccccc7)oc6c5c4c3)cc2)cc1 ; c1ccc(-c2ccc(N(c3ccc4c(c3)oc3ccccc34)c3ccc4ccc5ccc6nc(-c7ccccc7)oc6c5c4c3)cc2)cc1 ; CC1(C)c2ccccc2-c2ccc(N(c3ccc(-c4ccccc4)cc3)c3ccc(-c4ccc5c(ccc6ccc7sc(-c8ccccc8)nc7c65)c4)cc3)cc21]

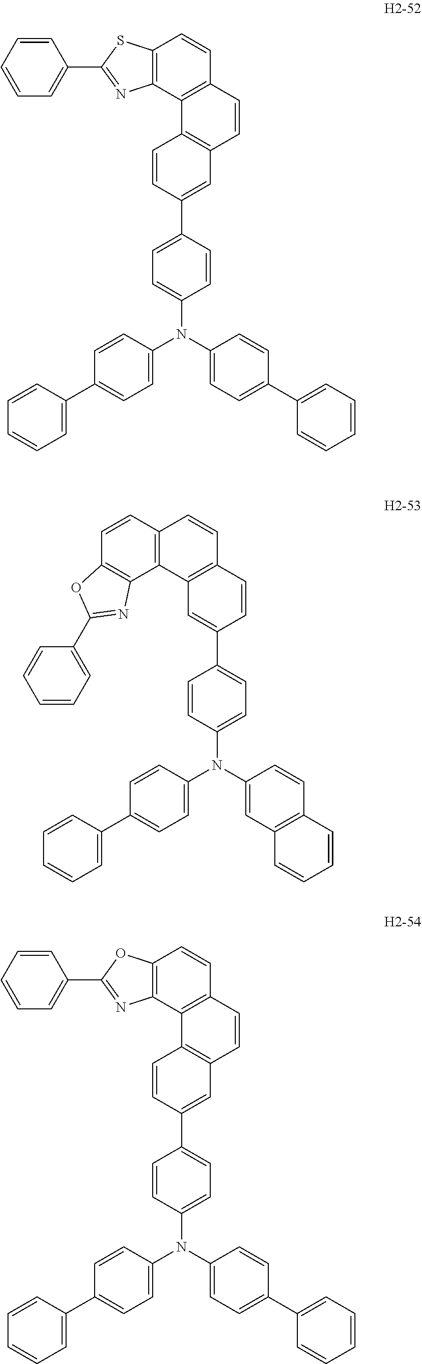 [SMILES: c1ccc(-c2ccc(N(c3ccc(-c4ccccc4)cc3)c3ccc(-c4ccc5c(ccc6ccc7sc(-c8ccccc8)nc7c65)c4)cc3)cc2)cc1 ; c1ccc(-c2ccc(N(c3ccc(-c4ccc5ccc6ccc7oc(-c8ccccc8)nc7c6c5c4)cc3)c3ccc4ccccc4c3)cc2)cc1 ; c1ccc(-c2ccc(N(c3ccc(-c4ccccc4)cc3)c3ccc(-c4ccc5c(ccc6ccc7oc(-c8ccccc8)nc7c65)c4)cc3)cc2)cc1]

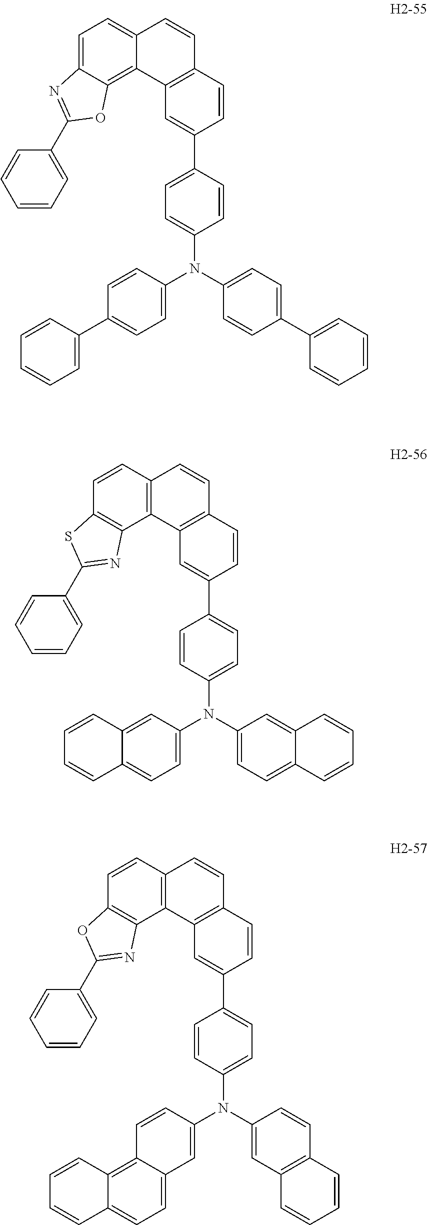 [SMILES: c1ccc(-c2ccc(N(c3ccc(-c4ccccc4)cc3)c3ccc(-c4ccc5ccc6ccc7nc(-c8ccccc8)oc7c6c5c4)cc3)cc2)cc1 ; c1ccc(-c2nc3c(ccc4ccc5ccc(-c6ccc(N(c7ccc8ccccc8c7)c7ccc8ccccc8c7)cc6)cc5c43)s2)cc1 ; c1ccc(-c2nc3c(ccc4ccc5ccc(-c6ccc(N(c7ccc8ccccc8c7)c7ccc8c(ccc9ccccc98)c7)cc6)cc5c43)o2)cc1]

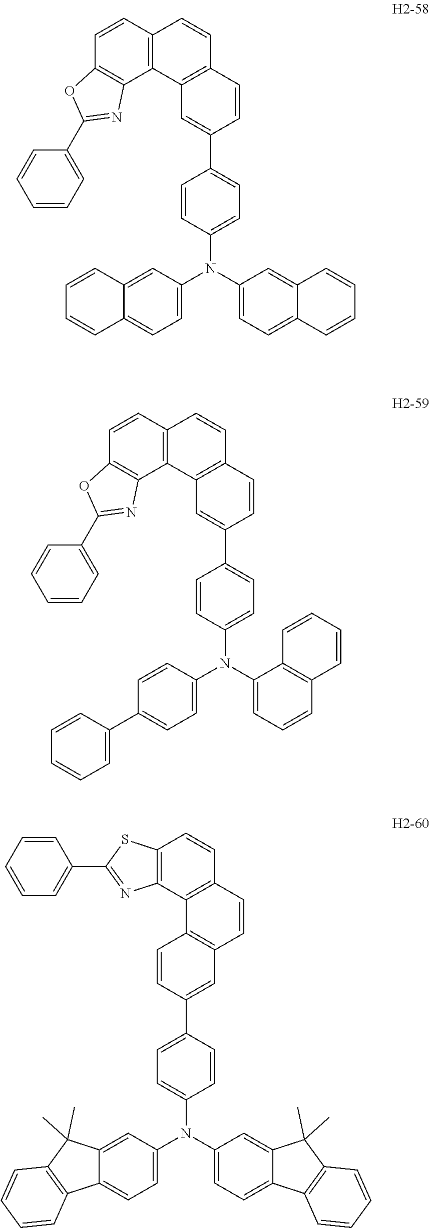 [SMILES: c1ccc(-c2nc3c(ccc4ccc5ccc(-c6ccc(N(c7ccc8ccccc8c7)c7ccc8ccccc8c7)cc6)cc5c43)o2)cc1 ; c1ccc(-c2ccc(N(c3ccc(-c4ccc5ccc6ccc7oc(-c8ccccc8)nc7c6c5c4)cc3)c3cccc4ccccc34)cc2)cc1 ; CC1(C)c2ccccc2-c2ccc(N(c3ccc(-c4ccc5c(ccc6ccc7sc(-c8ccccc8)nc7c65)c4)cc3)c3ccc4c(c3)C(C)(C)c3ccccc3-4)cc21]

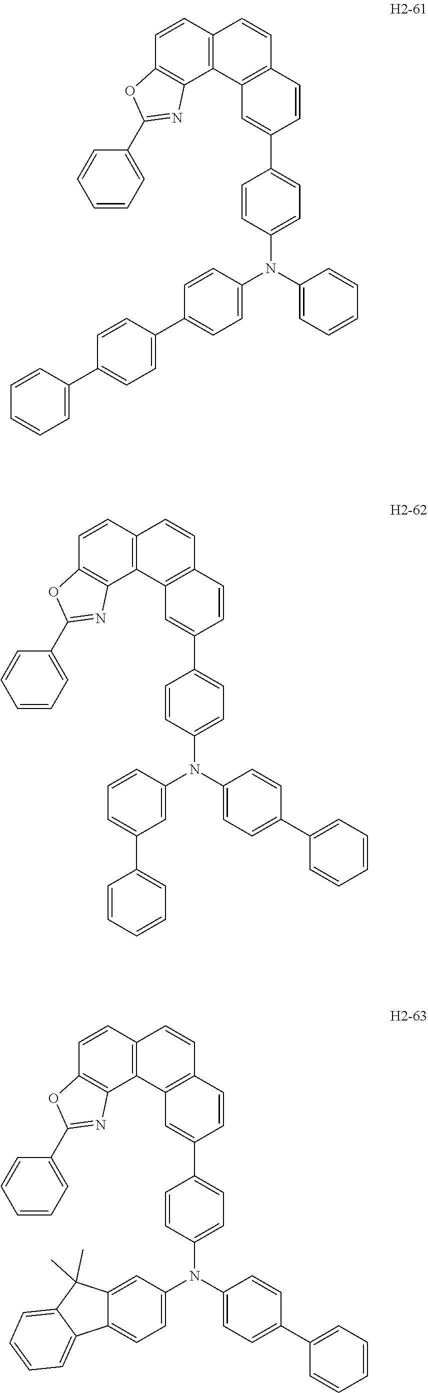 [SMILES: c1ccc(-c2ccc(-c3ccc(N(c4ccccc4)c4ccc(-c5ccc6ccc7ccc8oc(-c9ccccc9)nc8c7c6c5)cc4)cc3)cc2)cc1 ; c1ccc(-c2ccc(N(c3ccc(-c4ccc5ccc6ccc7oc(-c8ccccc8)nc7c6c5c4)cc3)c3cccc(-c4ccccc4)c3)cc2)cc1 ; CC1(C)c2ccccc2-c2ccc(N(c3ccc(-c4ccccc4)cc3)c3ccc(-c4ccc5ccc6ccc7oc(-c8ccccc8)nc7c6c5c4)cc3)cc21]

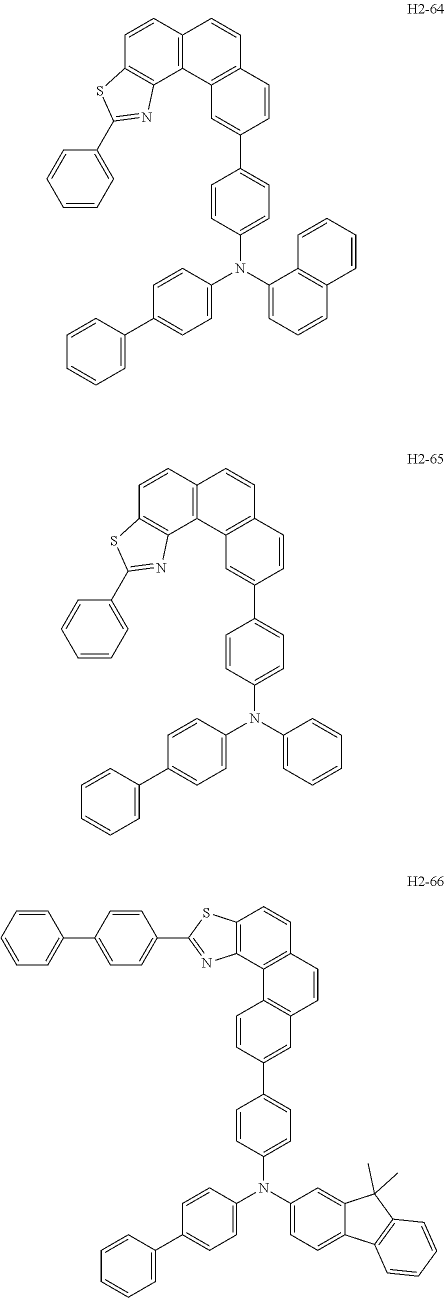 [SMILES: c1ccc(-c2ccc(N(c3ccc(-c4ccc5ccc6ccc7sc(-c8ccccc8)nc7c6c5c4)cc3)c3cccc4ccccc34)cc2)cc1 ; c1ccc(-c2ccc(N(c3ccccc3)c3ccc(-c4ccc5ccc6ccc7sc(-c8ccccc8)nc7c6c5c4)cc3)cc2)cc1 ; CC1(C)c2ccccc2-c2ccc(N(c3ccc(-c4ccccc4)cc3)c3ccc(-c4ccc5c(ccc6ccc7sc(-c8ccc(-c9ccccc9)cc8)nc7c65)c4)cc3)cc21]

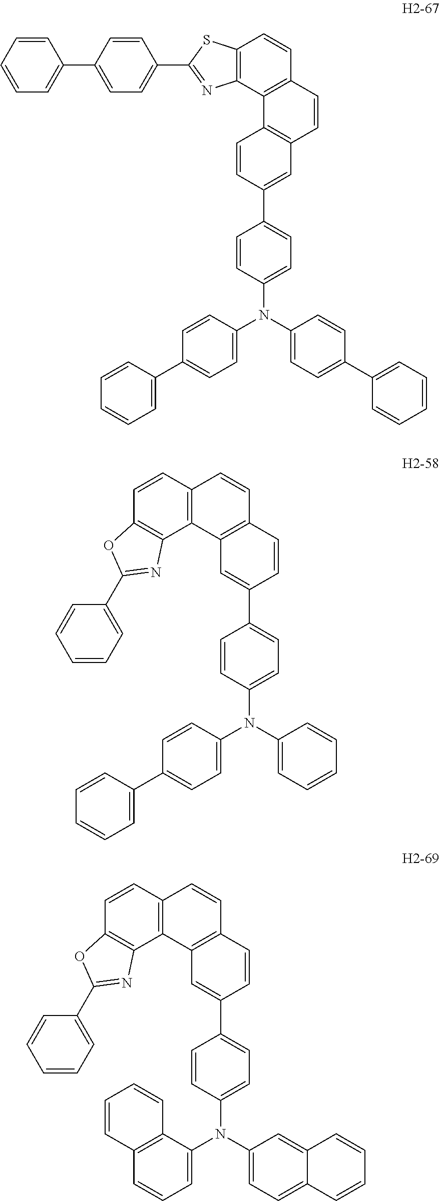 [SMILES: c1ccc(-c2ccc(-c3nc4c(ccc5ccc6cc(-c7ccc(N(c8ccc(-c9ccccc9)cc8)c8ccc(-c9ccccc9)cc8)cc7)ccc6c54)s3)cc2)cc1 ; c1ccc(-c2ccc(N(c3ccccc3)c3ccc(-c4ccc5ccc6ccc7oc(-c8ccccc8)nc7c6c5c4)cc3)cc2)cc1 ; c1ccc(-c2nc3c(ccc4ccc5ccc(-c6ccc(N(c7ccc8ccccc8c7)c7cccc8ccccc78)cc6)cc5c43)o2)cc1]

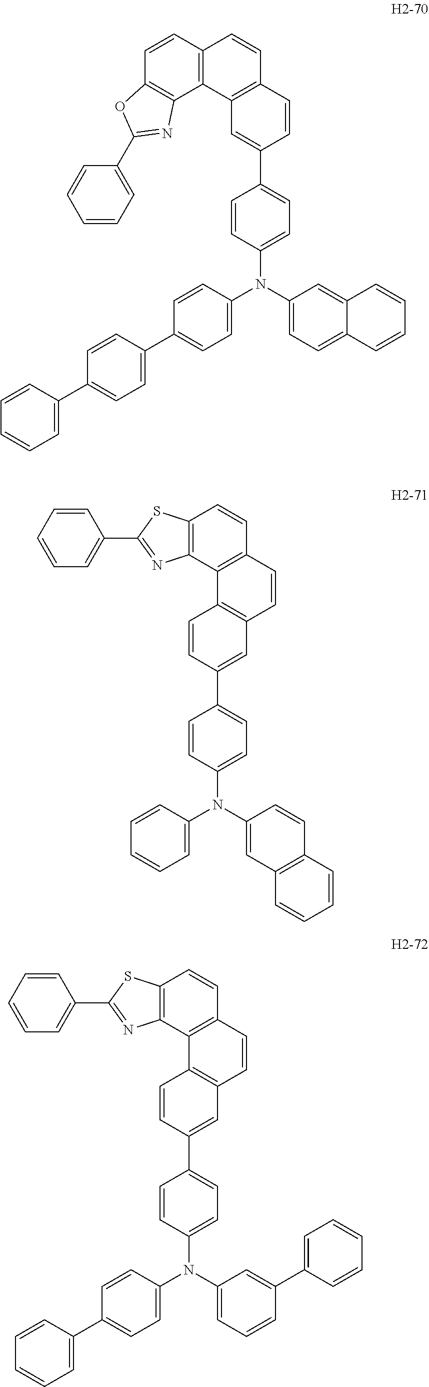 [SMILES: c1ccc(-c2ccc(-c3ccc(N(c4ccc(-c5ccc6ccc7ccc8oc(-c9ccccc9)nc8c7c6c5)cc4)c4ccc5ccccc5c4)cc3)cc2)cc1 ; c1ccc(-c2nc3c(ccc4ccc5cc(-c6ccc(N(c7ccccc7)c7ccc8ccccc8c7)cc6)ccc5c43)s2)cc1 ; c1ccc(-c2ccc(N(c3ccc(-c4ccc5c(ccc6ccc7sc(-c8ccccc8)nc7c65)c4)cc3)c3cccc(-c4ccccc4)c3)cc2)cc1]

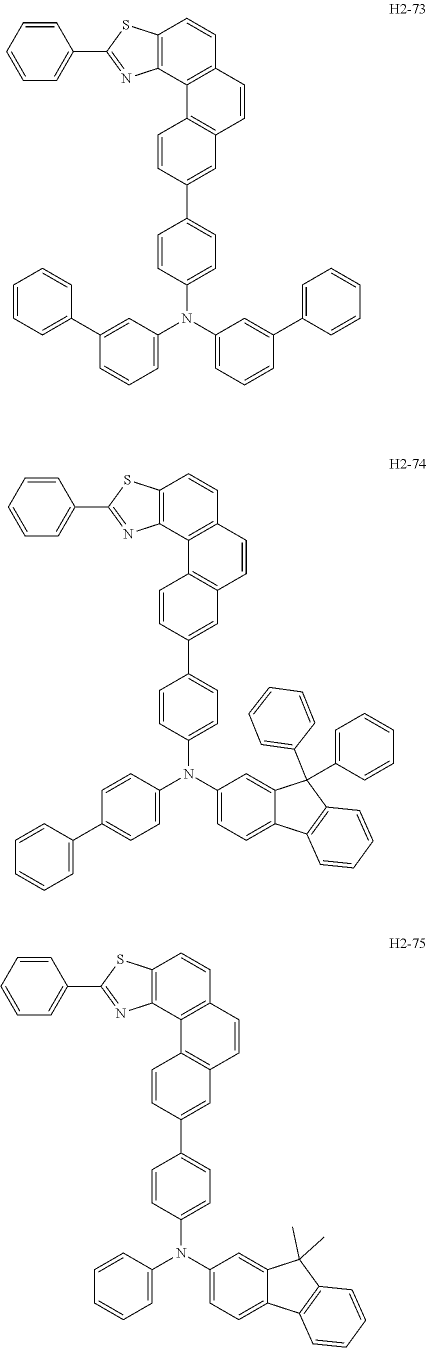 [SMILES: c1ccc(-c2cccc(N(c3ccc(-c4ccc5c(ccc6ccc7sc(-c8ccccc8)nc7c65)c4)cc3)c3cccc(-c4ccccc4)c3)c2)cc1 ; c1ccc(-c2ccc(N(c3ccc(-c4ccc5c(ccc6ccc7sc(-c8ccccc8)nc7c65)c4)cc3)c3ccc4c(c3)C(c3ccccc3)(c3ccccc3)c3ccccc3-4)cc2)cc1 ; CC1(C)c2ccccc2-c2ccc(N(c3ccccc3)c3ccc(-c4ccc5c(ccc6ccc7sc(-c8ccccc8)nc7c65)c4)cc3)cc21]

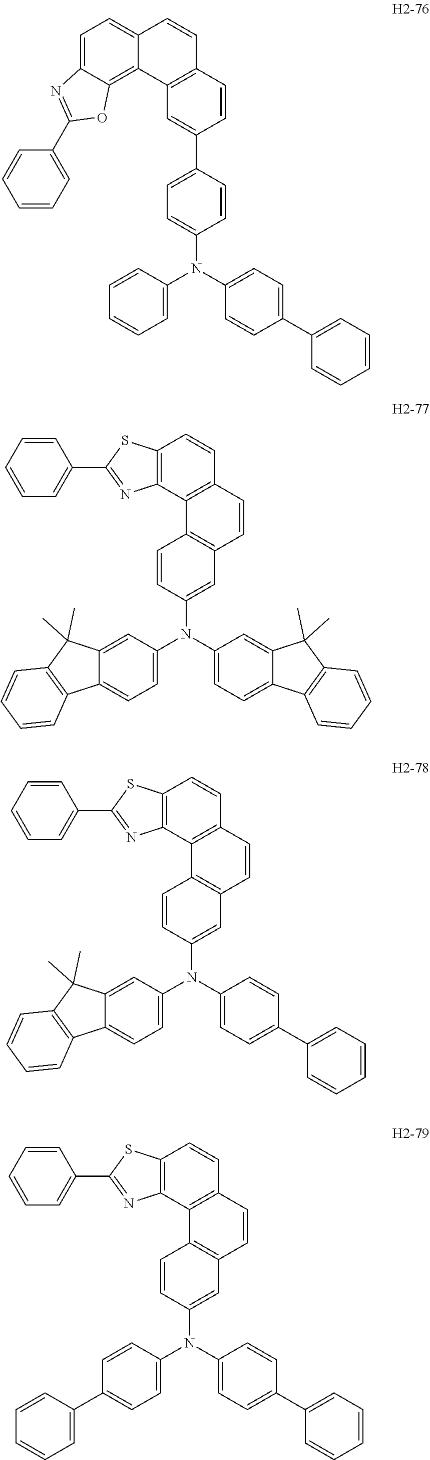 [SMILES: c1ccc(-c2ccc(N(c3ccccc3)c3ccc(-c4ccc5ccc6ccc7nc(-c8ccccc8)oc7c6c5c4)cc3)cc2)cc1 ; CC1(C)c2ccccc2-c2ccc(N(c3ccc4c(c3)C(C)(C)c3ccccc3-4)c3ccc4c(ccc5ccc6sc(-c7ccccc7)nc6c54)c3)cc21 ; CC1(C)c2ccccc2-c2ccc(N(c3ccc(-c4ccccc4)cc3)c3ccc4c(ccc5ccc6sc(-c7ccccc7)nc6c54)c3)cc21 ; c1ccc(-c2ccc(N(c3ccc(-c4ccccc4)cc3)c3ccc4c(ccc5ccc6sc(-c7ccccc7)nc6c54)c3)cc2)cc1]

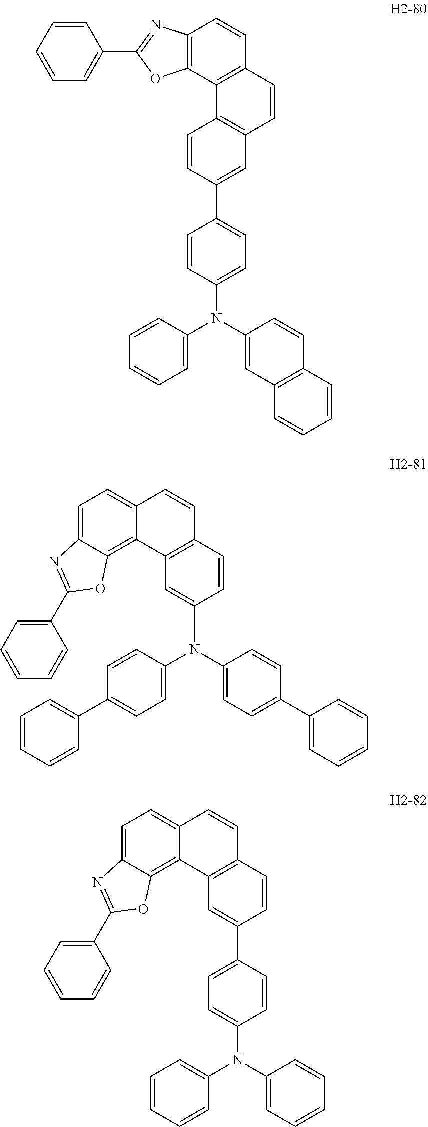 [SMILES: c1ccc(-c2nc3ccc4ccc5cc(-c6ccc(N(c7ccccc7)c7ccc8ccccc8c7)cc6)ccc5c4c3o2)cc1 ; c1ccc(-c2ccc(N(c3ccc(-c4ccccc4)cc3)c3ccc4ccc5ccc6nc(-c7ccccc7)oc6c5c4c3)cc2)cc1 ; c1ccc(-c2nc3ccc4ccc5ccc(-c6ccc(N(c7ccccc7)c7ccccc7)cc6)cc5c4c3o2)cc1]

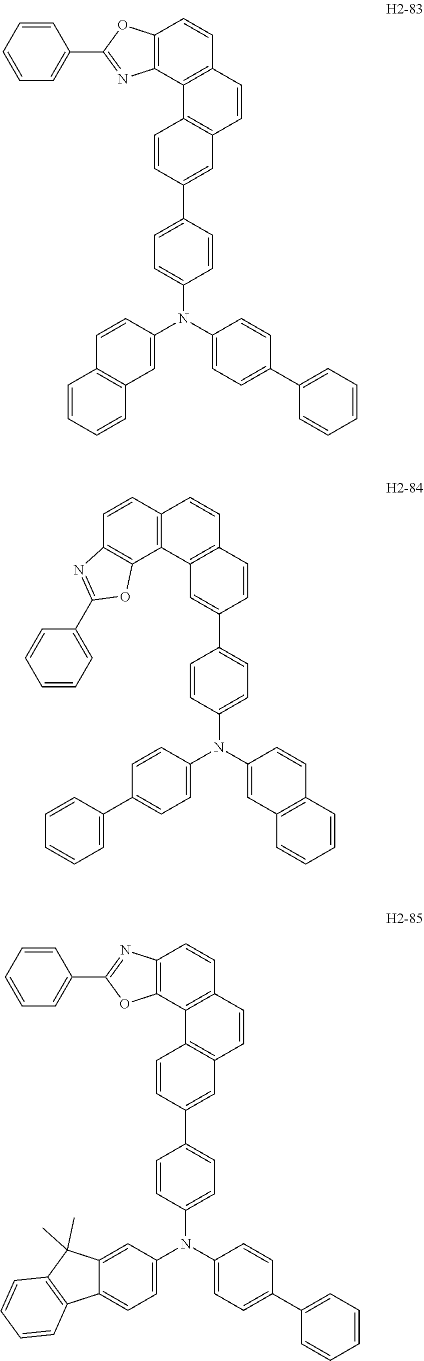 [SMILES: c1ccc(-c2ccc(N(c3ccc(-c4ccc5c(ccc6ccc7oc(-c8ccccc8)nc7c65)c4)cc3)c3ccc4ccccc4c3)cc2)cc1 ; c1ccc(-c2ccc(N(c3ccc(-c4ccc5ccc6ccc7nc(-c8ccccc8)oc7c6c5c4)cc3)c3ccc4ccccc4c3)cc2)cc1 ; CC1(C)c2ccccc2-c2ccc(N(c3ccc(-c4ccccc4)cc3)c3ccc(-c4ccc5c(ccc6ccc7nc(-c8ccccc8)oc7c65)c4)cc3)cc21]

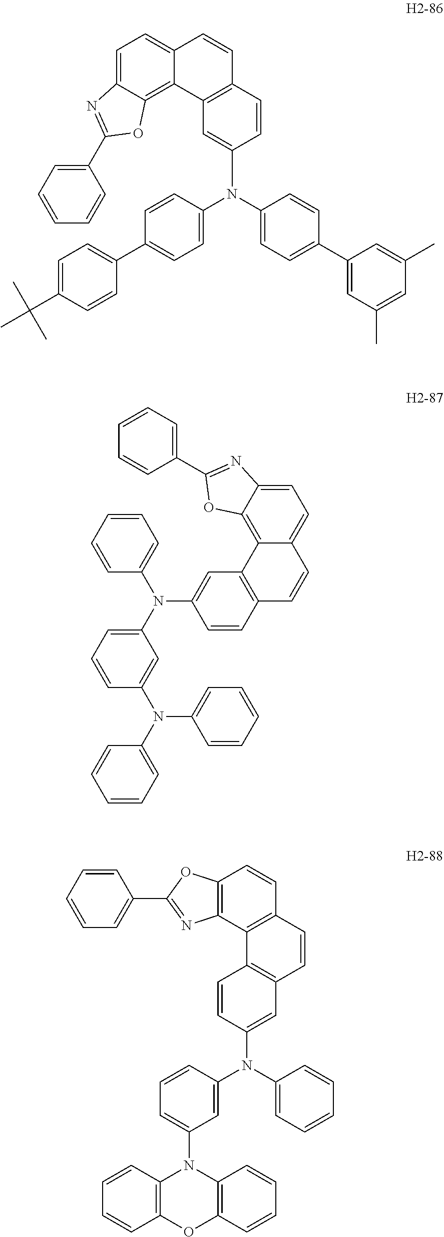 [SMILES: Cc1cc(C)cc(-c2ccc(N(c3ccc(-c4ccc(C(C)(C)C)cc4)cc3)c3ccc4ccc5ccc6nc(-c7ccccc7)oc6c5c4c3)cc2)c1 ; c1ccc(-c2nc3ccc4ccc5ccc(N(c6ccccc6)c6cccc(N(c7ccccc7)c7ccccc7)c6)cc5c4c3o2)cc1 ; c1ccc(-c2nc3c(ccc4ccc5cc(N(c6ccccc6)c6cccc(N7c8ccccc8Oc8ccccc87)c6)ccc5c43)o2)cc1]

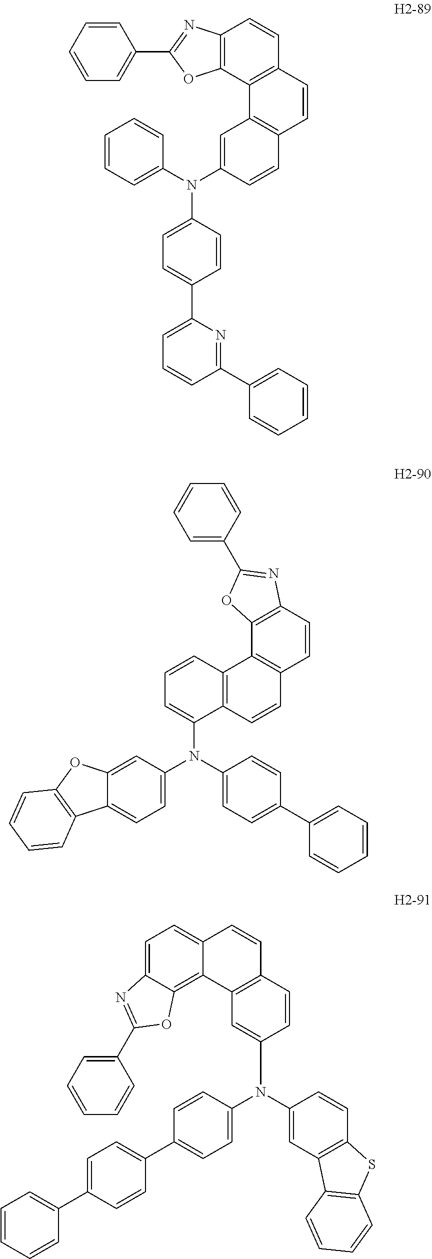 [SMILES: c1ccc(-c2cccc(-c3ccc(N(c4ccccc4)c4ccc5ccc6ccc7nc(-c8ccccc8)oc7c6c5c4)cc3)n2)cc1 ; c1ccc(-c2ccc(N(c3ccc4c(c3)oc3ccccc34)c3cccc4c3ccc3ccc5nc(-c6ccccc6)oc5c34)cc2)cc1 ; c1ccc(-c2ccc(-c3ccc(N(c4ccc5sc6ccccc6c5c4)c4ccc5ccc6ccc7nc(-c8ccccc8)oc7c6c5c4)cc3)cc2)cc1]

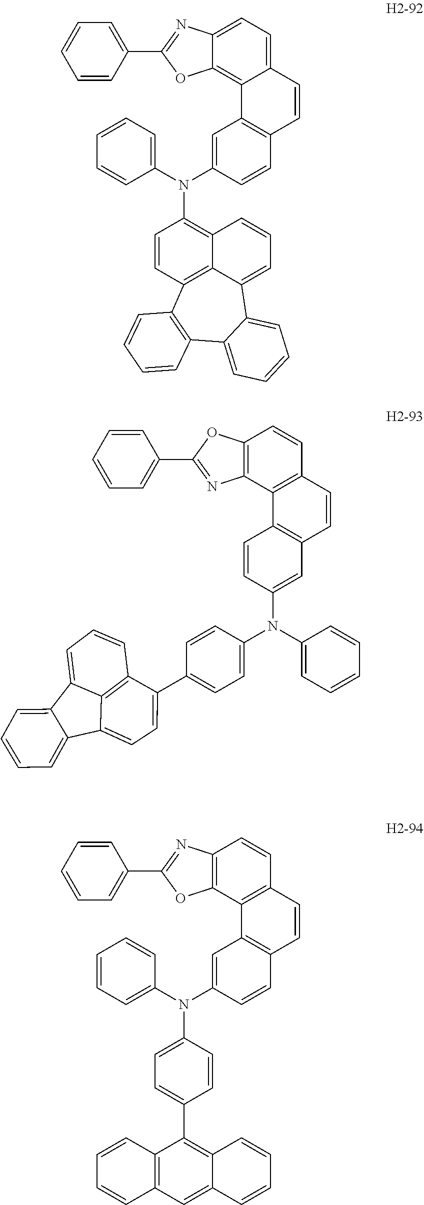

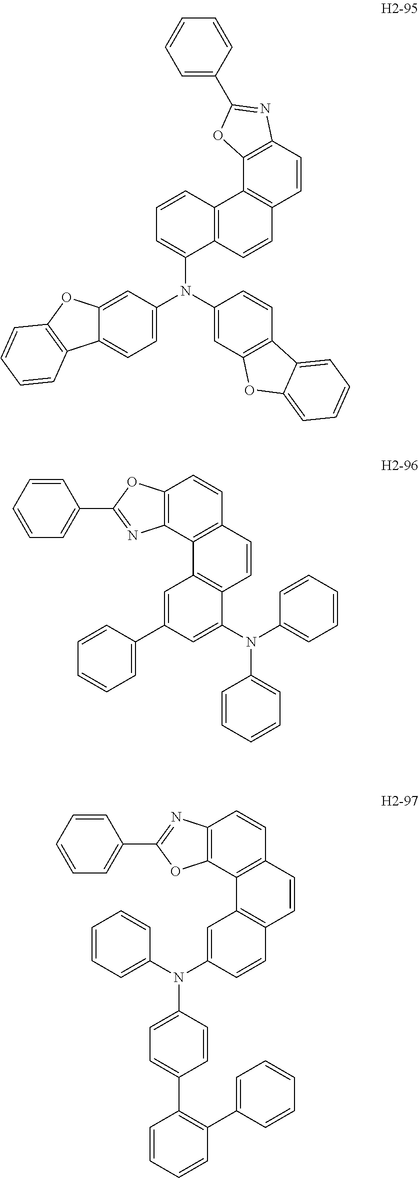 [SMILES: c1ccc(-c2nc3ccc4ccc5c(N(c6ccc7c(c6)oc6ccccc67)c6ccc7c(c6)oc6ccccc67)cccc5c4c3o2)cc1 ; c1ccc(-c2cc(N(c3ccccc3)c3ccccc3)c3ccc4ccc5oc(-c6ccccc6)nc5c4c3c2)cc1 ; c1ccc(-c2nc3ccc4ccc5ccc(N(c6ccccc6)c6ccc(-c7ccccc7-c7ccccc7)cc6)cc5c4c3o2)cc1]

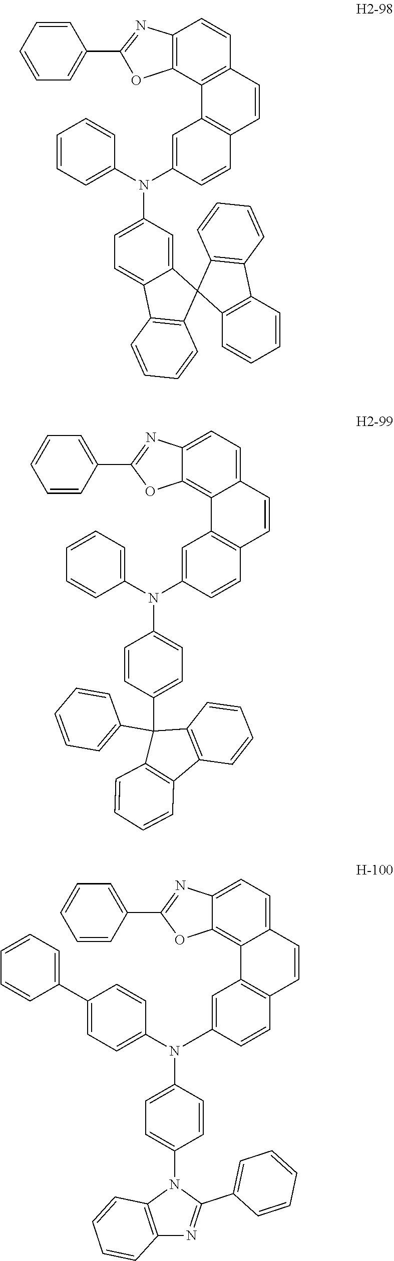

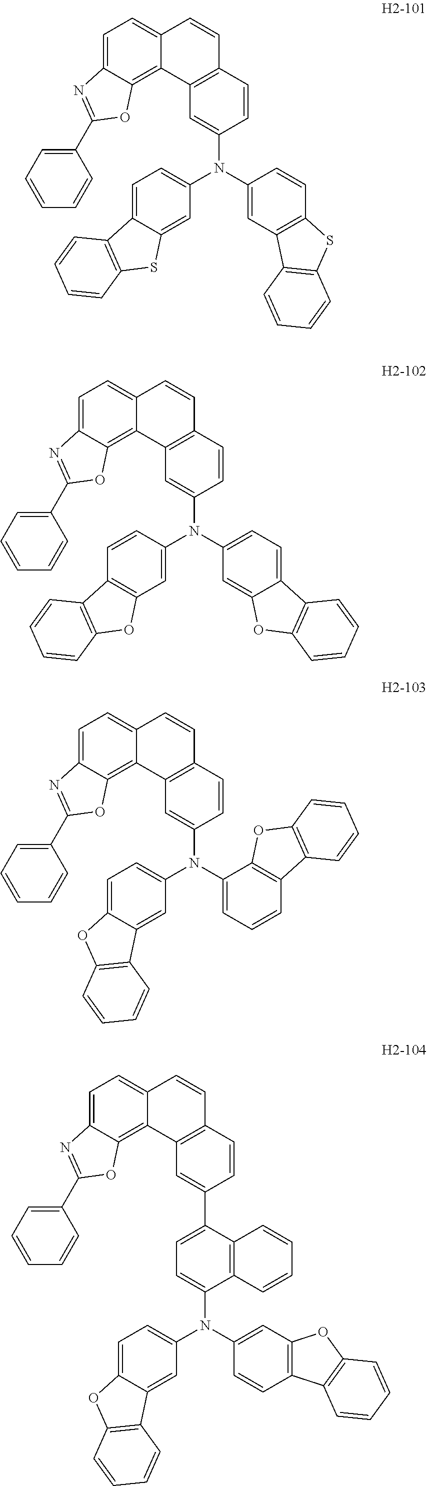 [SMILES: c1ccc(-c2nc3ccc4ccc5ccc(N(c6ccc7c(c6)sc6ccccc67)c6ccc7sc8ccccc8c7c6)cc5c4c3o2)cc1 ; c1ccc(-c2nc3ccc4ccc5ccc(N(c6ccc7c(c6)oc6ccccc67)c6ccc7c(c6)oc6ccccc67)cc5c4c3o2)cc1 ; c1ccc(-c2nc3ccc4ccc5ccc(N(c6ccc7oc8ccccc8c7c6)c6cccc7c6oc6ccccc67)cc5c4c3o2)cc1 ; c1ccc(-c2nc3ccc4ccc5ccc(-c6ccc(N(c7ccc8c(c7)oc7ccccc78)c7ccc8oc9ccccc9c8c7)c7ccccc67)cc5c4c3o2)cc1]

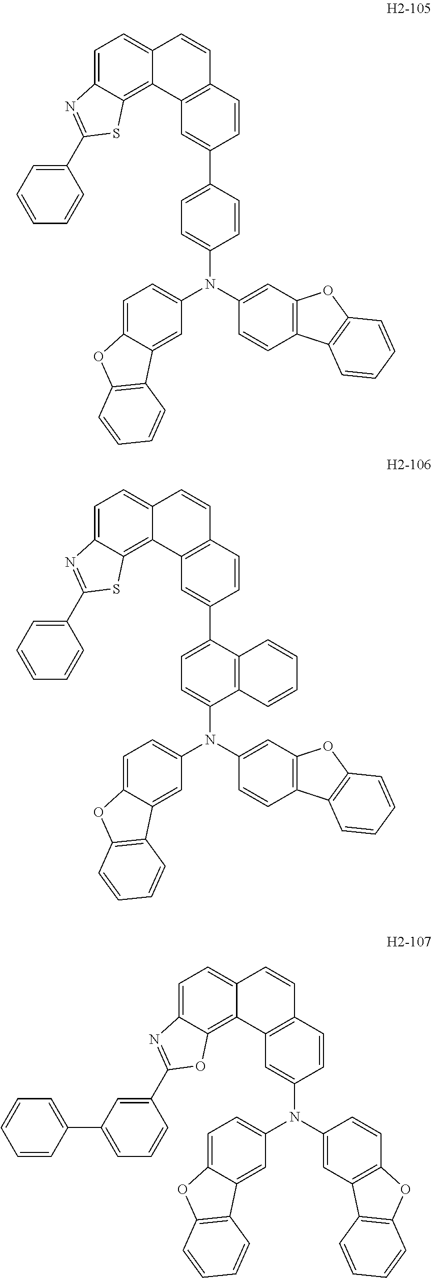

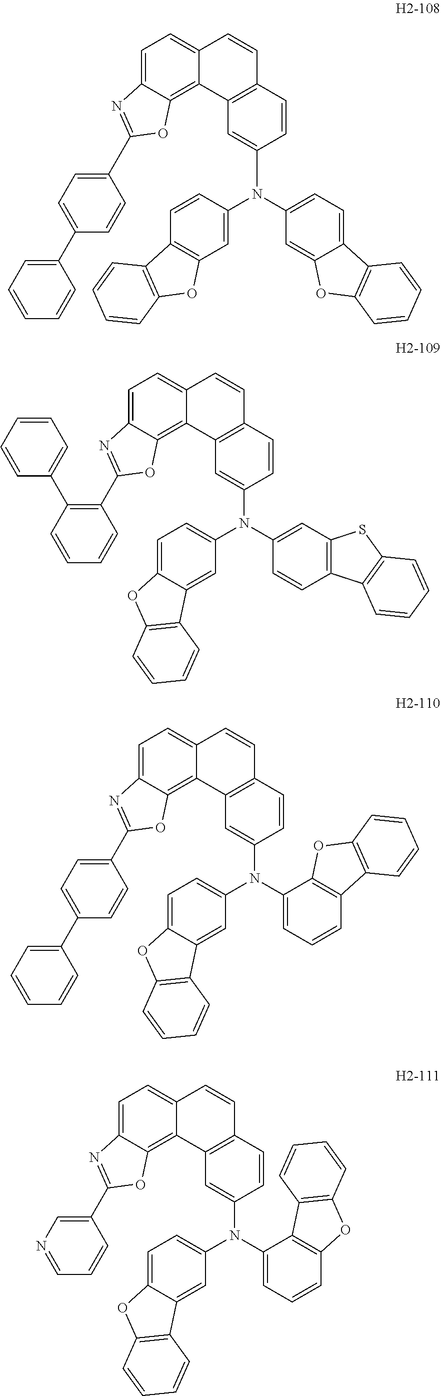 [SMILES: c1ccc(-c2ccc(-c3nc4ccc5ccc6ccc(N(c7ccc8c(c7)oc7ccccc78)c7ccc8c(c7)oc7ccccc78)cc6c5c4o3)cc2)cc1 ; c1ccc(-c2ccccc2-c2nc3ccc4ccc5ccc(N(c6ccc7c(c6)sc6ccccc67)c6ccc7oc8ccccc8c7c6)cc5c4c3o2)cc1 ; c1ccc(-c2ccc(-c3nc4ccc5ccc6ccc(N(c7ccc8oc9ccccc9c8c7)c7cccc8c7oc7ccccc78)cc6c5c4o3)cc2)cc1 ; c1cncc(-c2nc3ccc4ccc5ccc(N(c6ccc7oc8ccccc8c7c6)c6cccc7oc8ccccc8c67)cc5c4c3o2)c1]

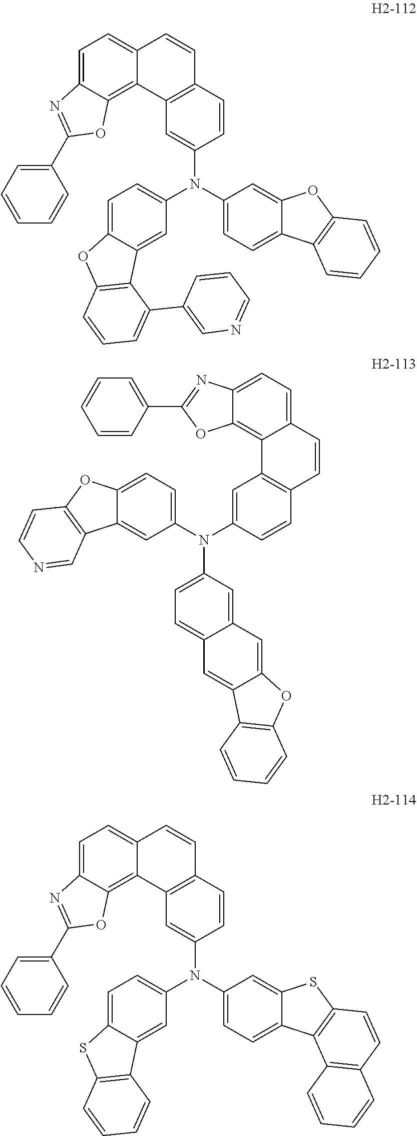

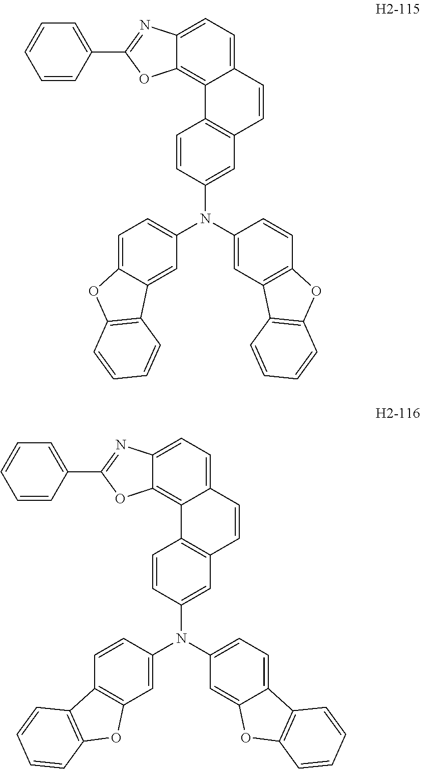 [SMILES: c1ccc(-c2nc3ccc4ccc5cc(N(c6ccc7oc8ccccc8c7c6)c6ccc7oc8ccccc8c7c6)ccc5c4c3o2)cc1 ; c1ccc(-c2nc3ccc4ccc5cc(N(c6ccc7c(c6)oc6ccccc67)c6ccc7c(c6)oc6ccccc67)ccc5c4c3o2)cc1]

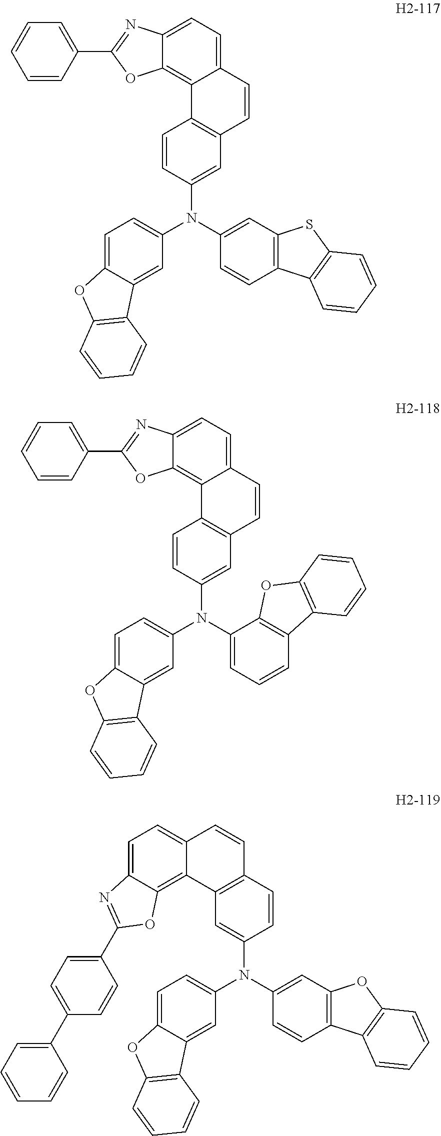 [SMILES: c1ccc(-c2nc3ccc4ccc5cc(N(c6ccc7c(c6)sc6ccccc67)c6ccc7oc8ccccc8c7c6)ccc5c4c3o2)cc1 ; c1ccc(-c2nc3ccc4ccc5cc(N(c6ccc7oc8ccccc8c7c6)c6cccc7c6oc6ccccc67)ccc5c4c3o2)cc1 ; c1ccc(-c2ccc(-c3nc4ccc5ccc6ccc(N(c7ccc8c(c7)oc7ccccc78)c7ccc8oc9ccccc9c8c7)cc6c5c4o3)cc2)cc1]

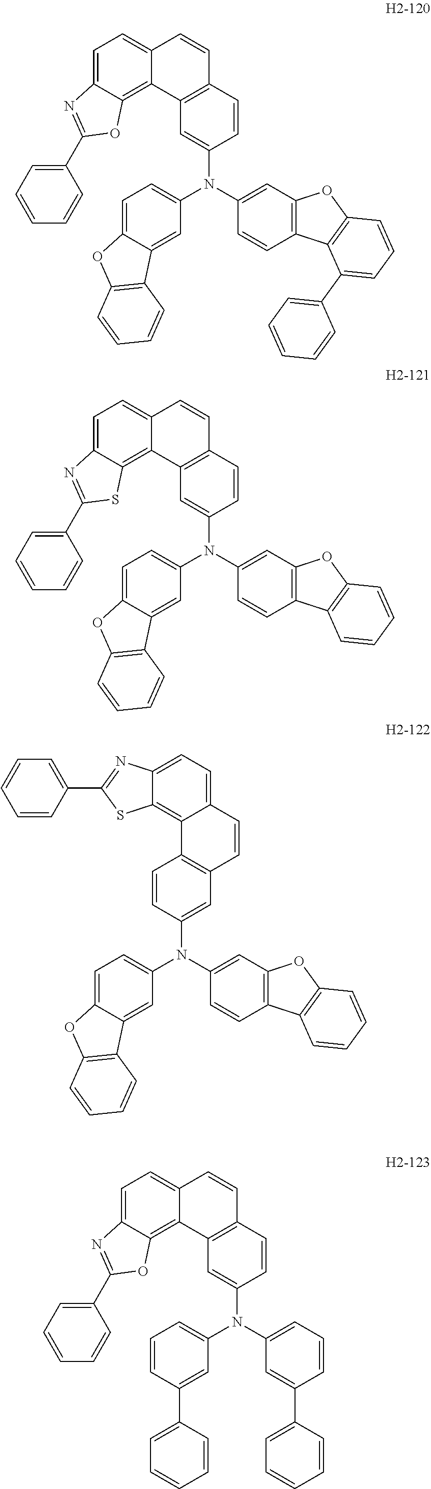 [SMILES: c1ccc(-c2nc3ccc4ccc5ccc(N(c6ccc7c(c6)oc6cccc(-c8ccccc8)c67)c6ccc7oc8ccccc8c7c6)cc5c4c3o2)cc1 ; c1ccc(-c2nc3ccc4ccc5ccc(N(c6ccc7c(c6)oc6ccccc67)c6ccc7oc8ccccc8c7c6)cc5c4c3s2)cc1 ; c1ccc(-c2nc3ccc4ccc5cc(N(c6ccc7c(c6)oc6ccccc67)c6ccc7oc8ccccc8c7c6)ccc5c4c3s2)cc1 ; c1ccc(-c2cccc(N(c3cccc(-c4ccccc4)c3)c3ccc4ccc5ccc6nc(-c7ccccc7)oc6c5c4c3)c2)cc1]

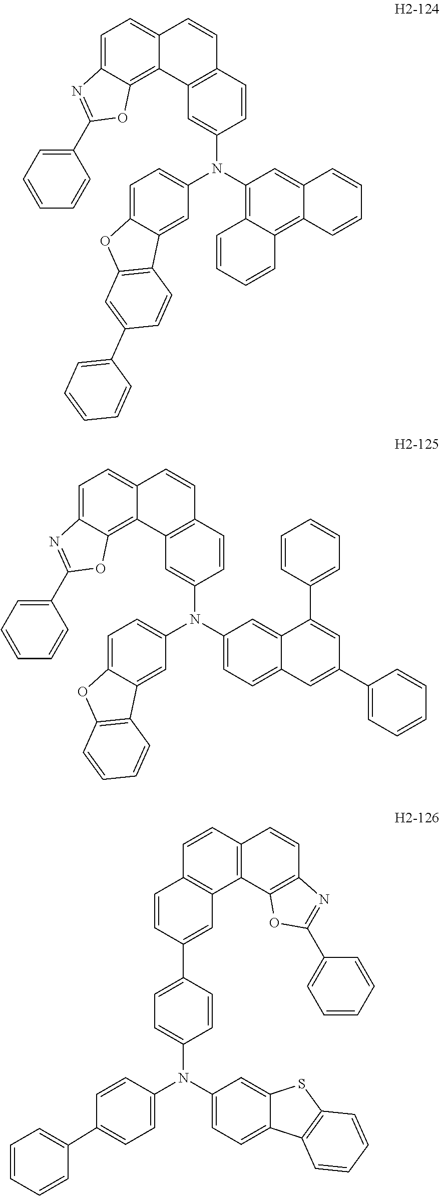 [SMILES: c1ccc(-c2ccc3c(c2)oc2ccc(N(c4ccc5ccc6ccc7nc(-c8ccccc8)oc7c6c5c4)c4cc5ccccc5c5ccccc45)cc23)cc1 ; c1ccc(-c2cc(-c3ccccc3)c3cc(N(c4ccc5oc6ccccc6c5c4)c4ccc5ccc6ccc7nc(-c8ccccc8)oc7c6c5c4)ccc3c2)cc1 ; Cc1c(/N=C(\O)c2ccccc2)ccc2ccc3ccc(-c4ccc(N(c5ccc(-c6ccccc6)cc5)c5ccc6c(c5)sc5ccccc56)cc4)cc3c12]

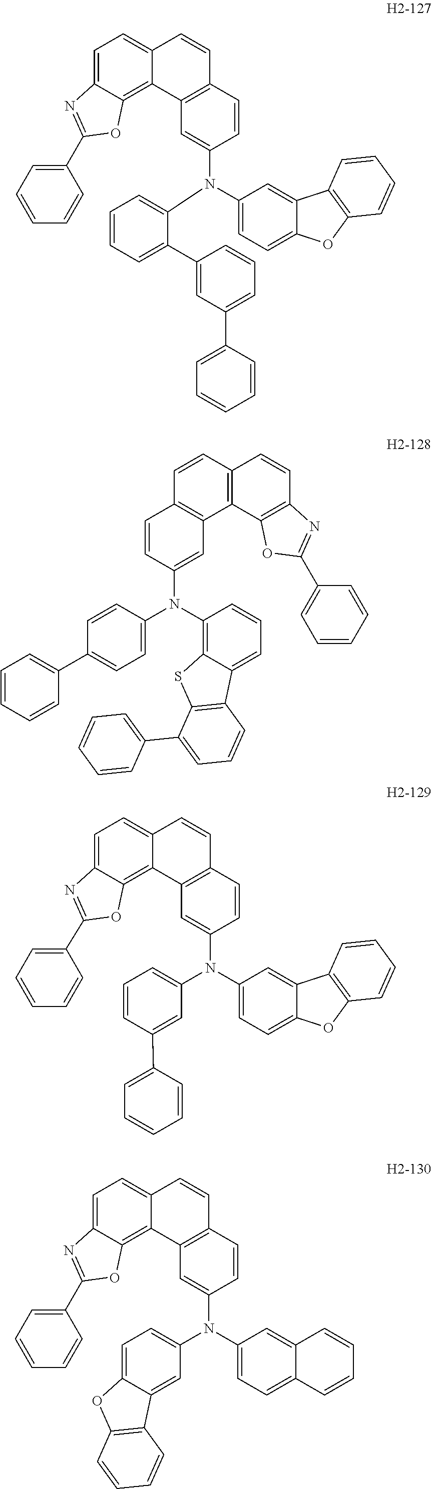 [SMILES: c1ccc(-c2cccc(-c3ccccc3N(c3ccc4oc5ccccc5c4c3)c3ccc4ccc5ccc6nc(-c7ccccc7)oc6c5c4c3)c2)cc1 ; c1ccc(-c2ccc(N(c3ccc4ccc5ccc6nc(-c7ccccc7)oc6c5c4c3)c3cccc4c3sc3c(-c5ccccc5)cccc34)cc2)cc1 ; c1ccc(-c2cccc(N(c3ccc4oc5ccccc5c4c3)c3ccc4ccc5ccc6nc(-c7ccccc7)oc6c5c4c3)c2)cc1 ; c1ccc(-c2nc3ccc4ccc5ccc(N(c6ccc7ccccc7c6)c6ccc7oc8ccccc8c7c6)cc5c4c3o2)cc1]

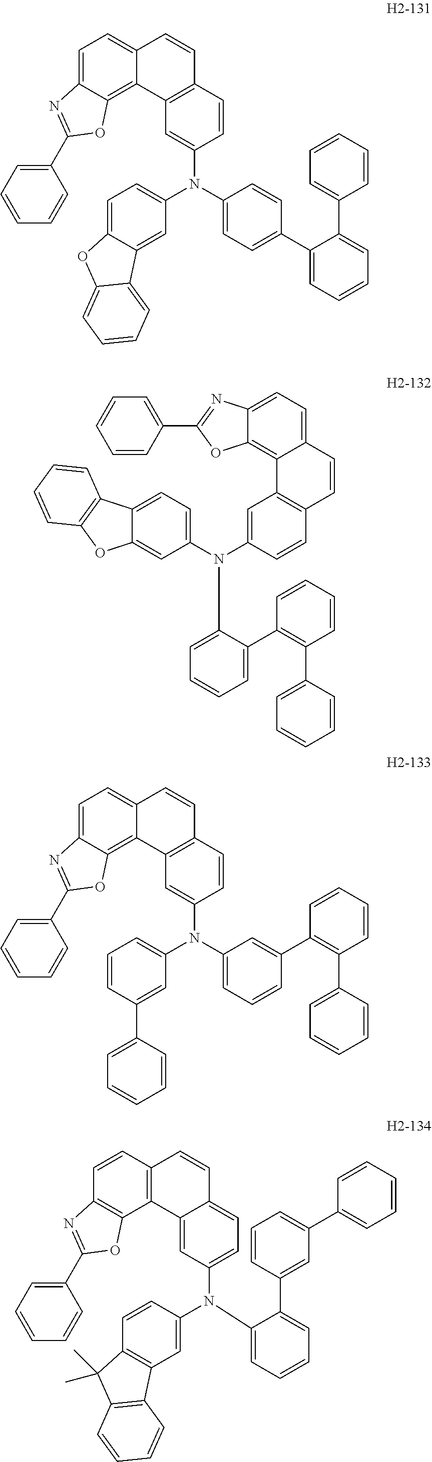 [SMILES: c1ccc(-c2nc3ccc4ccc5ccc(N(c6ccc(-c7ccccc7-c7ccccc7)cc6)c6ccc7oc8ccccc8c7c6)cc5c4c3o2)cc1 ; c1ccc(-c2nc3ccc4ccc5ccc(N(c6ccc7c(c6)oc6ccccc67)c6ccccc6-c6ccccc6-c6ccccc6)cc5c4c3o2)cc1 ; c1ccc(-c2cccc(N(c3cccc(-c4ccccc4-c4ccccc4)c3)c3ccc4ccc5ccc6nc(-c7ccccc7)oc6c5c4c3)c2)cc1 ; CC1(C)c2ccccc2-c2cc(N(c3ccc4ccc5ccc6nc(-c7ccccc7)oc6c5c4c3)c3ccccc3-c3cccc(-c4ccccc4)c3)ccc21]

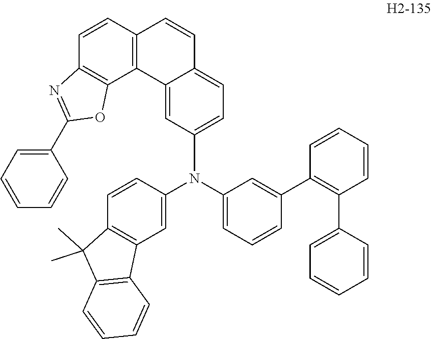 [SMILES: CC1(C)c2ccccc2-c2cc(N(c3cccc(-c4ccccc4-c4ccccc4)c3)c3ccc4ccc5ccc6nc(-c7ccccc7)oc6c5c4c3)ccc21]

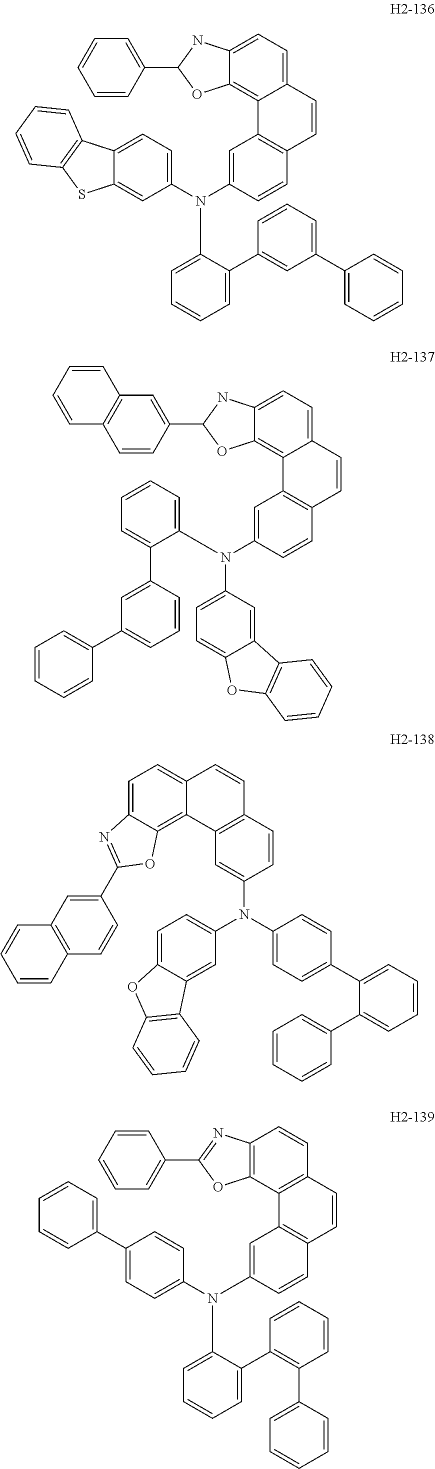

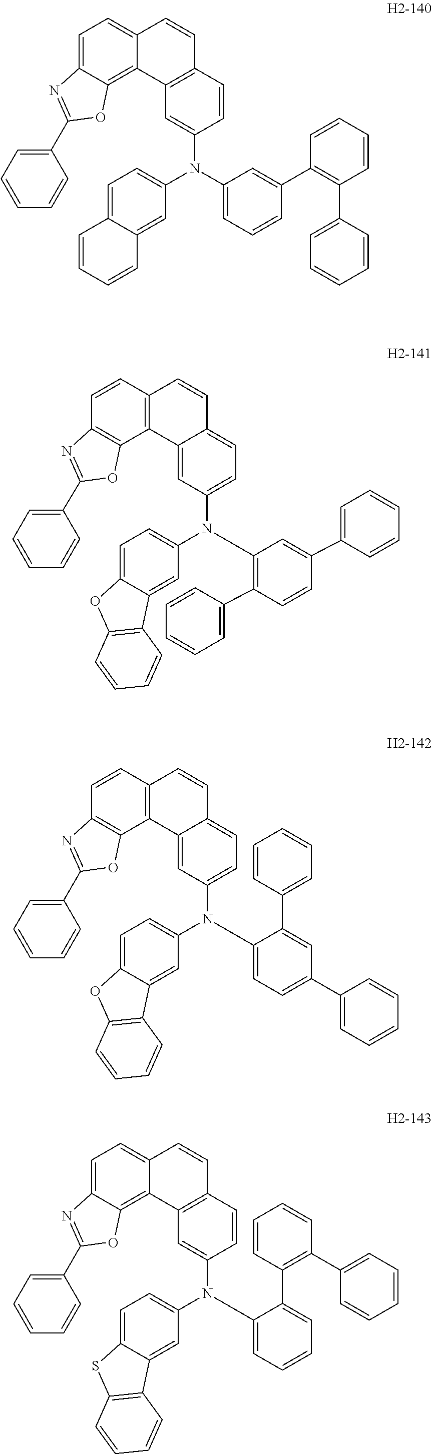 [SMILES: c1ccc(-c2nc3ccc4ccc5ccc(N(c6cccc(-c7ccccc7-c7ccccc7)c6)c6ccc7ccccc7c6)cc5c4c3o2)cc1 ; c1ccc(-c2ccc(-c3ccccc3)c(N(c3ccc4oc5ccccc5c4c3)c3ccc4ccc5ccc6nc(-c7ccccc7)oc6c5c4c3)c2)cc1 ; c1ccc(-c2ccc(N(c3ccc4oc5ccccc5c4c3)c3ccc4ccc5ccc6nc(-c7ccccc7)oc6c5c4c3)c(-c3ccccc3)c2)cc1 ; c1ccc(-c2nc3ccc4ccc5ccc(N(c6ccc7sc8ccccc8c7c6)c6ccccc6-c6ccccc6-c6ccccc6)cc5c4c3o2)cc1]

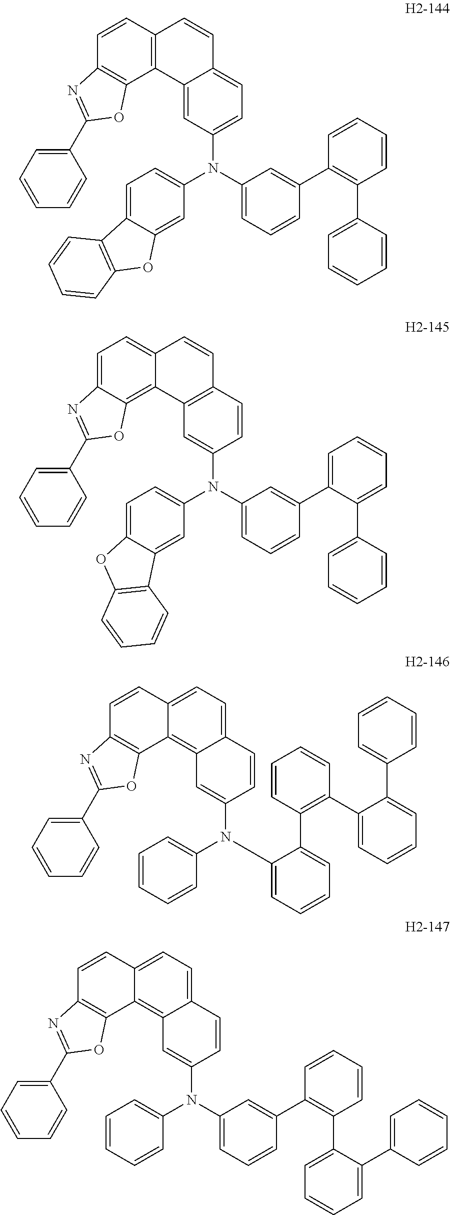

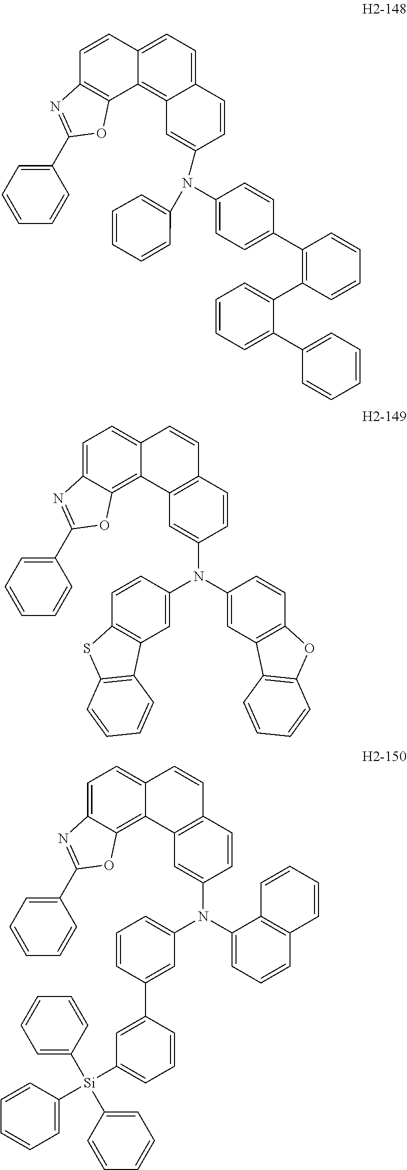

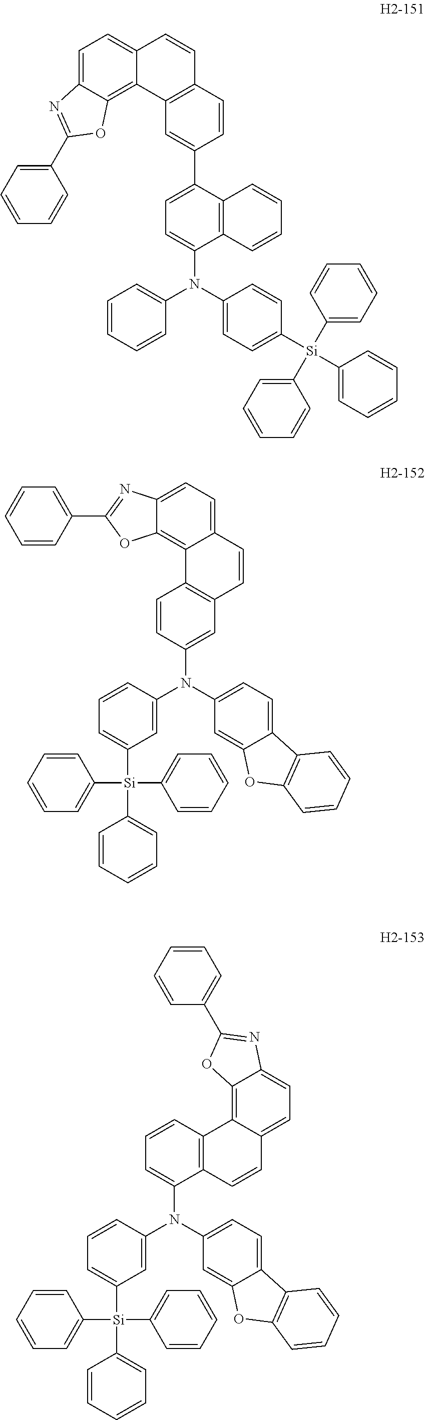 [SMILES: c1ccc(-c2nc3ccc4ccc5ccc(-c6ccc(N(c7ccccc7)c7ccc([Si](c8ccccc8)(c8ccccc8)c8ccccc8)cc7)c7ccccc67)cc5c4c3o2)cc1 ; c1ccc(-c2nc3ccc4ccc5cc(N(c6cccc([Si](c7ccccc7)(c7ccccc7)c7ccccc7)c6)c6ccc7c(c6)oc6ccccc67)ccc5c4c3o2)cc1 ; c1ccc(-c2nc3ccc4ccc5c(N(c6cccc([Si](c7ccccc7)(c7ccccc7)c7ccccc7)c6)c6ccc7c(c6)oc6ccccc67)cccc5c4c3o2)cc1]

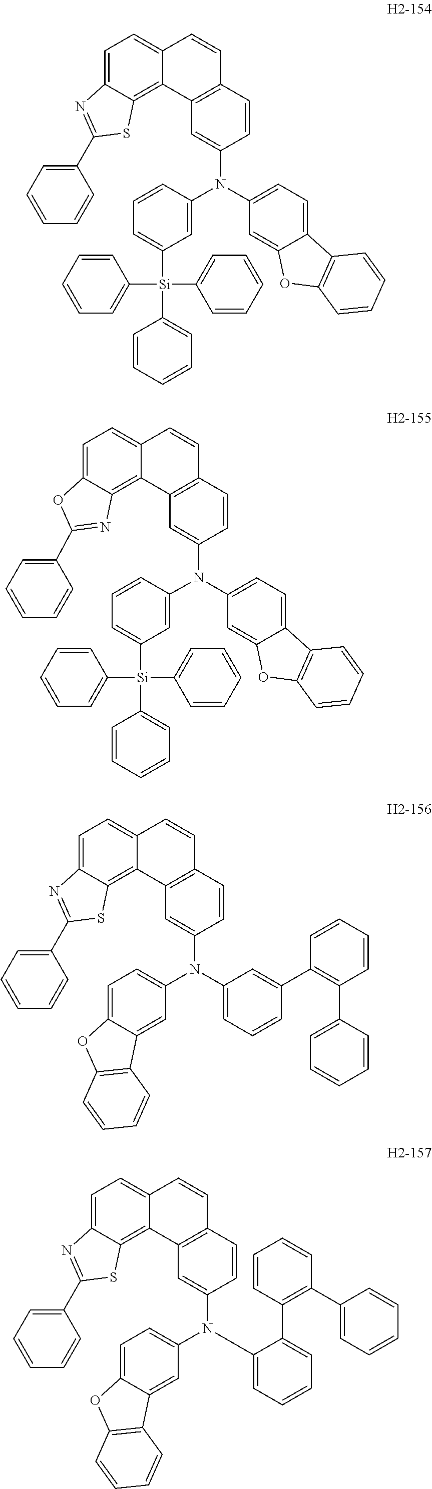

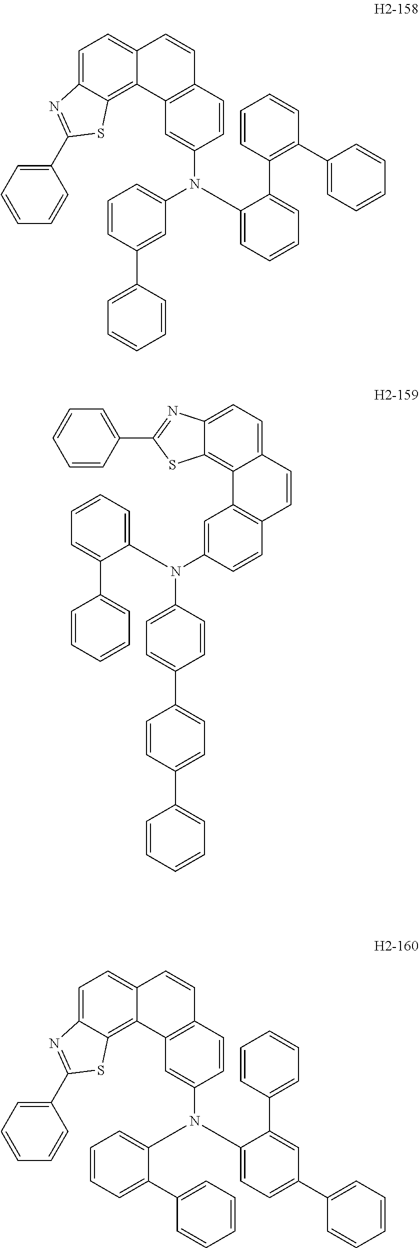 [SMILES: c1ccc(-c2cccc(N(c3ccc4ccc5ccc6nc(-c7ccccc7)sc6c5c4c3)c3ccccc3-c3ccccc3-c3ccccc3)c2)cc1 ; c1ccc(-c2ccc(-c3ccc(N(c4ccc5ccc6ccc7nc(-c8ccccc8)sc7c6c5c4)c4ccccc4-c4ccccc4)cc3)cc2)cc1 ; c1ccc(-c2ccc(N(c3ccc4ccc5ccc6nc(-c7ccccc7)sc6c5c4c3)c3ccccc3-c3ccccc3)c(-c3ccccc3)c2)cc1]

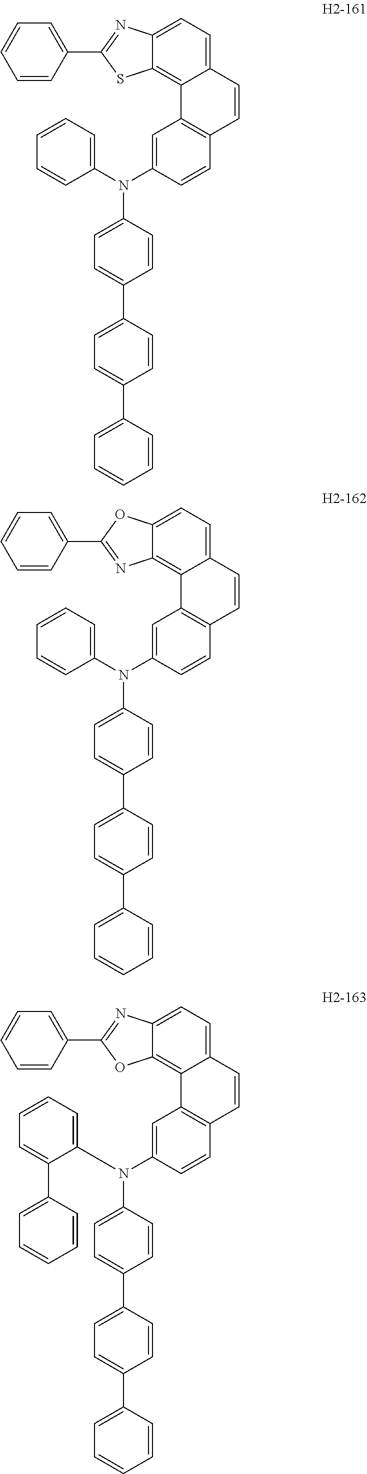

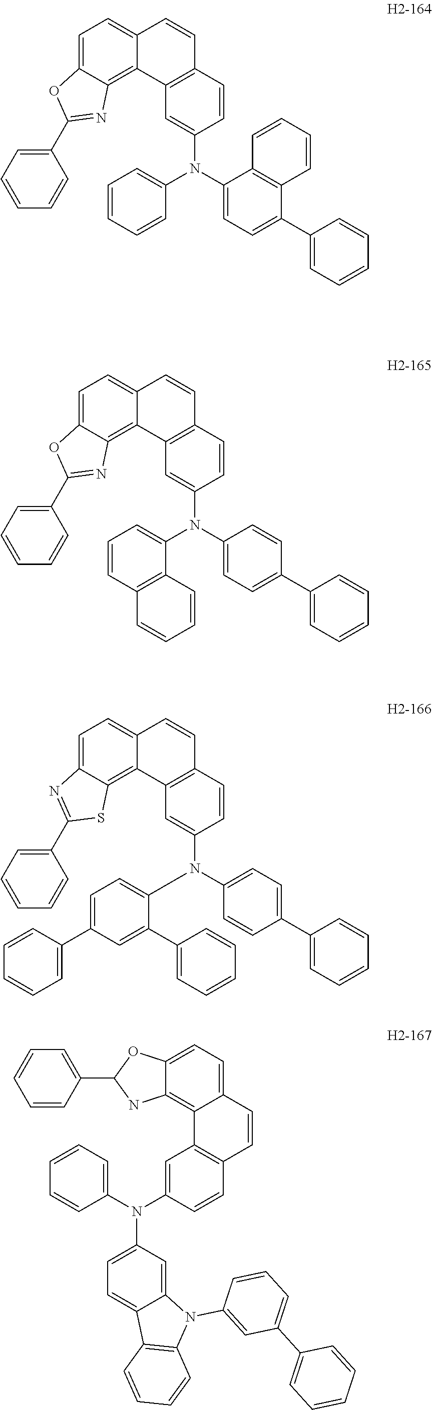

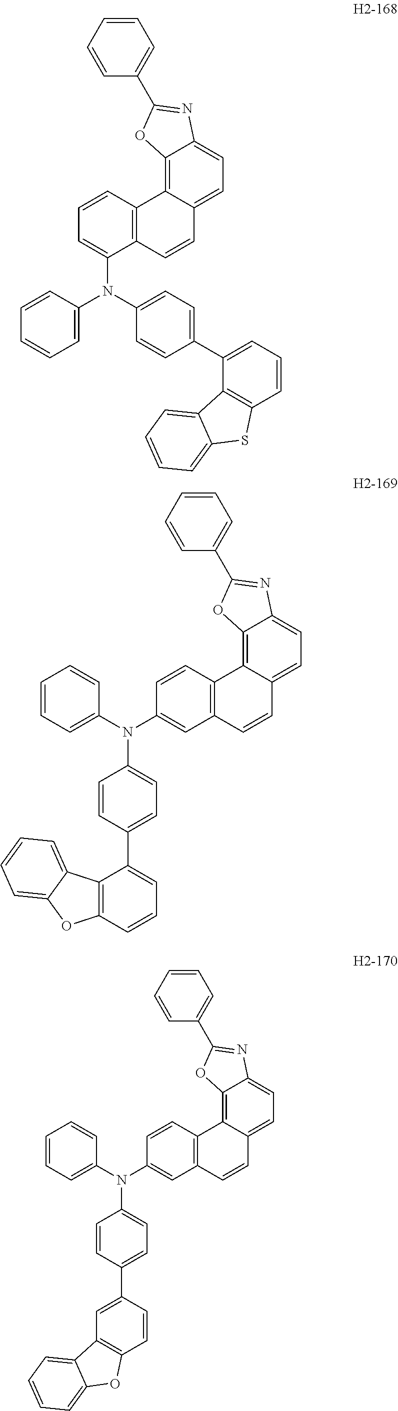

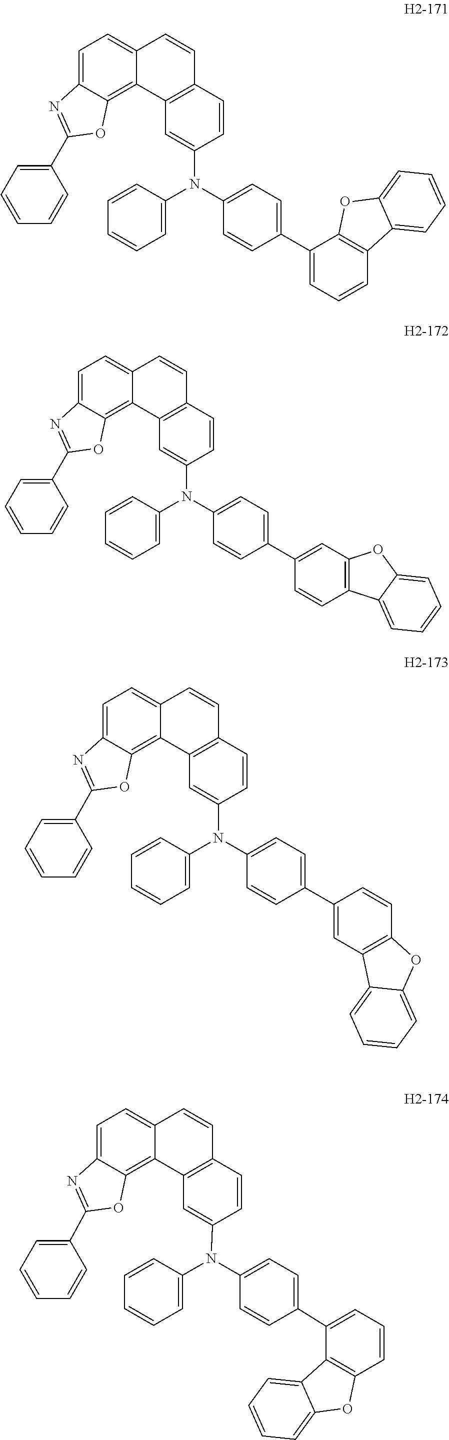

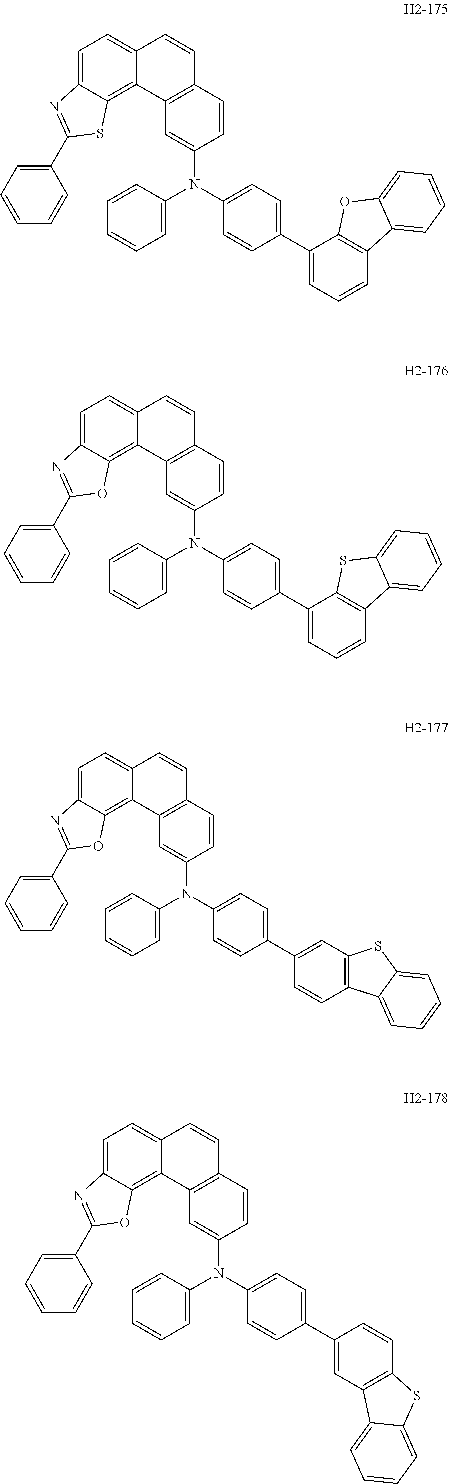 [SMILES: c1ccc(-c2nc3ccc4ccc5ccc(N(c6ccccc6)c6ccc(-c7cccc8c7oc7ccccc78)cc6)cc5c4c3s2)cc1 ; c1ccc(-c2nc3ccc4ccc5ccc(N(c6ccccc6)c6ccc(-c7cccc8c7sc7ccccc78)cc6)cc5c4c3o2)cc1 ; c1ccc(-c2nc3ccc4ccc5ccc(N(c6ccccc6)c6ccc(-c7ccc8c(c7)sc7ccccc78)cc6)cc5c4c3o2)cc1 ; c1ccc(-c2nc3ccc4ccc5ccc(N(c6ccccc6)c6ccc(-c7ccc8sc9ccccc9c8c7)cc6)cc5c4c3o2)cc1]

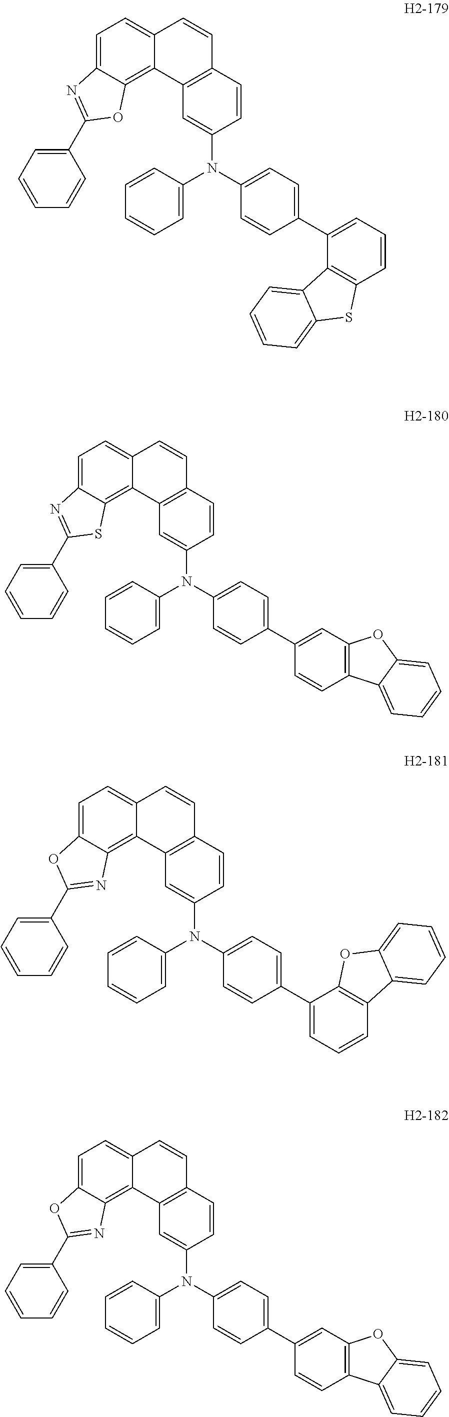 [SMILES: c1ccc(-c2nc3ccc4ccc5ccc(N(c6ccccc6)c6ccc(-c7cccc8sc9ccccc9c78)cc6)cc5c4c3o2)cc1 ; c1ccc(-c2nc3ccc4ccc5ccc(N(c6ccccc6)c6ccc(-c7ccc8c(c7)oc7ccccc78)cc6)cc5c4c3s2)cc1 ; c1ccc(-c2nc3c(ccc4ccc5ccc(N(c6ccccc6)c6ccc(-c7cccc8c7oc7ccccc78)cc6)cc5c43)o2)cc1 ; c1ccc(-c2nc3c(ccc4ccc5ccc(N(c6ccccc6)c6ccc(-c7ccc8c(c7)oc7ccccc78)cc6)cc5c43)o2)cc1]

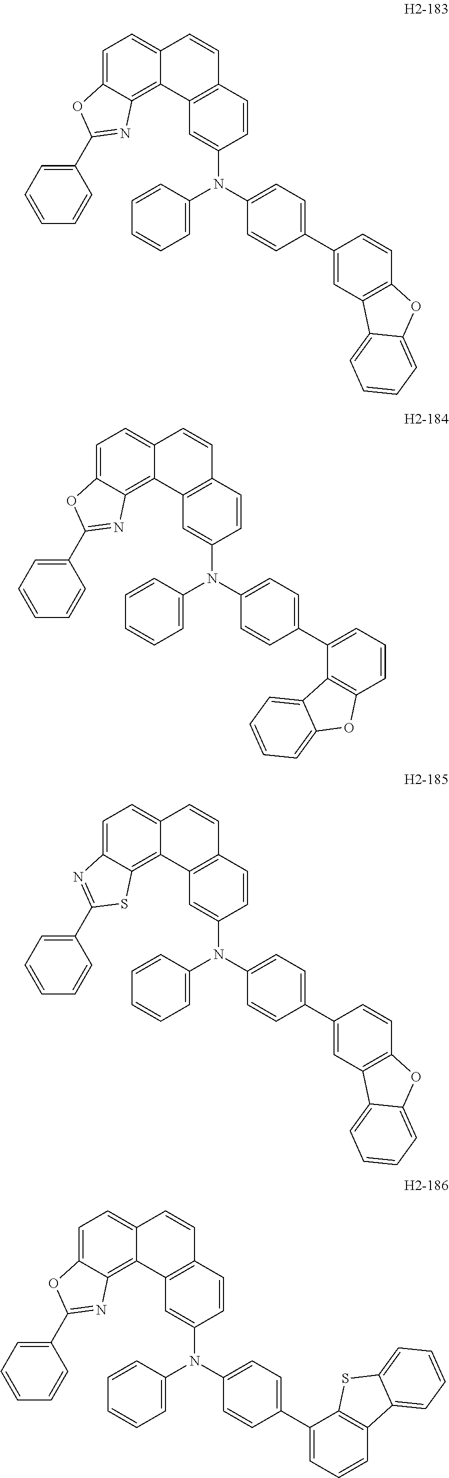 [SMILES: c1ccc(-c2nc3c(ccc4ccc5ccc(N(c6ccccc6)c6ccc(-c7ccc8oc9ccccc9c8c7)cc6)cc5c43)o2)cc1 ; c1ccc(-c2nc3c(ccc4ccc5ccc(N(c6ccccc6)c6ccc(-c7cccc8oc9ccccc9c78)cc6)cc5c43)o2)cc1 ; c1ccc(-c2nc3ccc4ccc5ccc(N(c6ccccc6)c6ccc(-c7ccc8oc9ccccc9c8c7)cc6)cc5c4c3s2)cc1 ; c1ccc(-c2nc3c(ccc4ccc5ccc(N(c6ccccc6)c6ccc(-c7cccc8c7sc7ccccc78)cc6)cc5c43)o2)cc1]

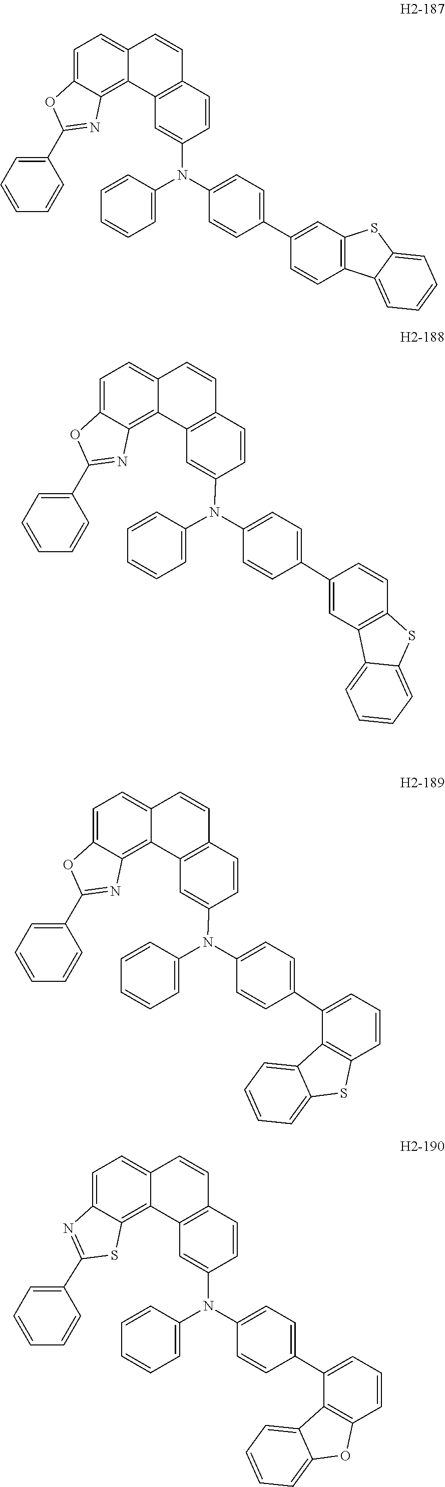 [SMILES: c1ccc(-c2nc3c(ccc4ccc5ccc(N(c6ccccc6)c6ccc(-c7ccc8c(c7)sc7ccccc78)cc6)cc5c43)o2)cc1 ; c1ccc(-c2nc3c(ccc4ccc5ccc(N(c6ccccc6)c6ccc(-c7ccc8sc9ccccc9c8c7)cc6)cc5c43)o2)cc1 ; c1ccc(-c2nc3c(ccc4ccc5ccc(N(c6ccccc6)c6ccc(-c7cccc8sc9ccccc9c78)cc6)cc5c43)o2)cc1 ; c1ccc(-c2nc3ccc4ccc5ccc(N(c6ccccc6)c6ccc(-c7cccc8oc9ccccc9c78)cc6)cc5c4c3s2)cc1]

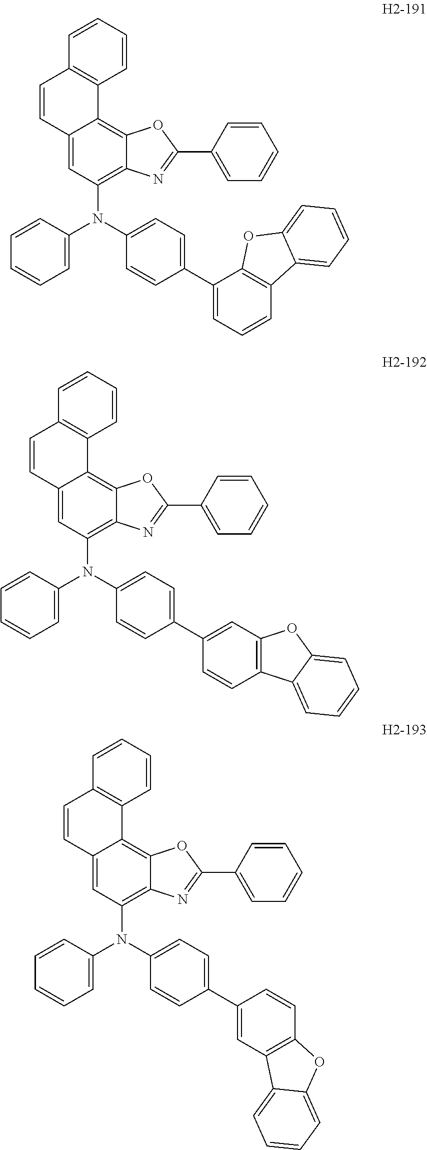 [SMILES: c1ccc(-c2nc3c(N(c4ccccc4)c4ccc(-c5cccc6c5oc5ccccc56)cc4)cc4ccc5ccccc5c4c3o2)cc1 ; c1ccc(-c2nc3c(N(c4ccccc4)c4ccc(-c5ccc6c(c5)oc5ccccc56)cc4)cc4ccc5ccccc5c4c3o2)cc1 ; c1ccc(-c2nc3c(N(c4ccccc4)c4ccc(-c5ccc6oc7ccccc7c6c5)cc4)cc4ccc5ccccc5c4c3o2)cc1]

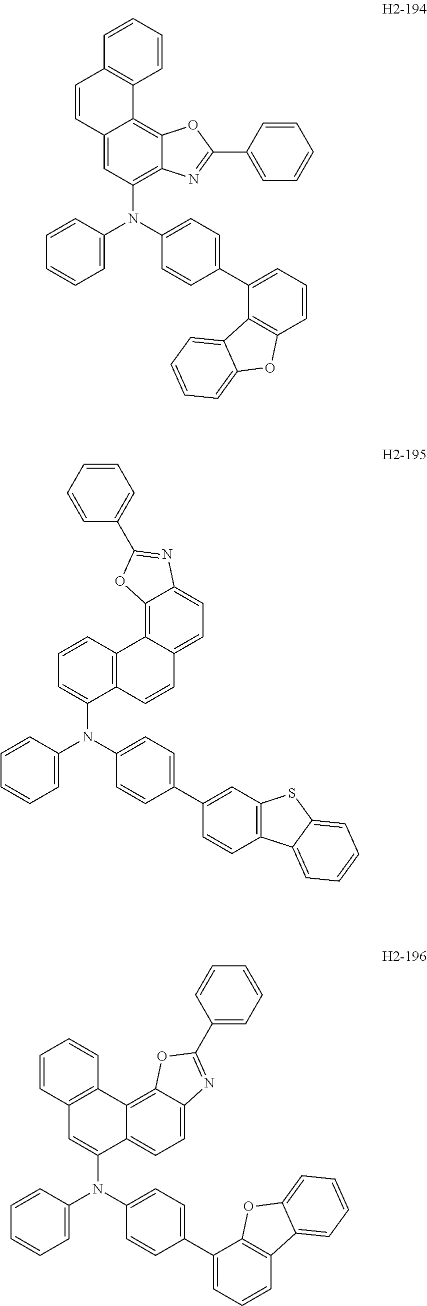 [SMILES: c1ccc(-c2nc3c(N(c4ccccc4)c4ccc(-c5cccc6oc7ccccc7c56)cc4)cc4ccc5ccccc5c4c3o2)cc1 ; c1ccc(-c2nc3ccc4ccc5c(N(c6ccccc6)c6ccc(-c7ccc8c(c7)sc7ccccc78)cc6)cccc5c4c3o2)cc1 ; c1ccc(-c2nc3ccc4c(N(c5ccccc5)c5ccc(-c6cccc7c6oc6ccccc67)cc5)cc5ccccc5c4c3o2)cc1]

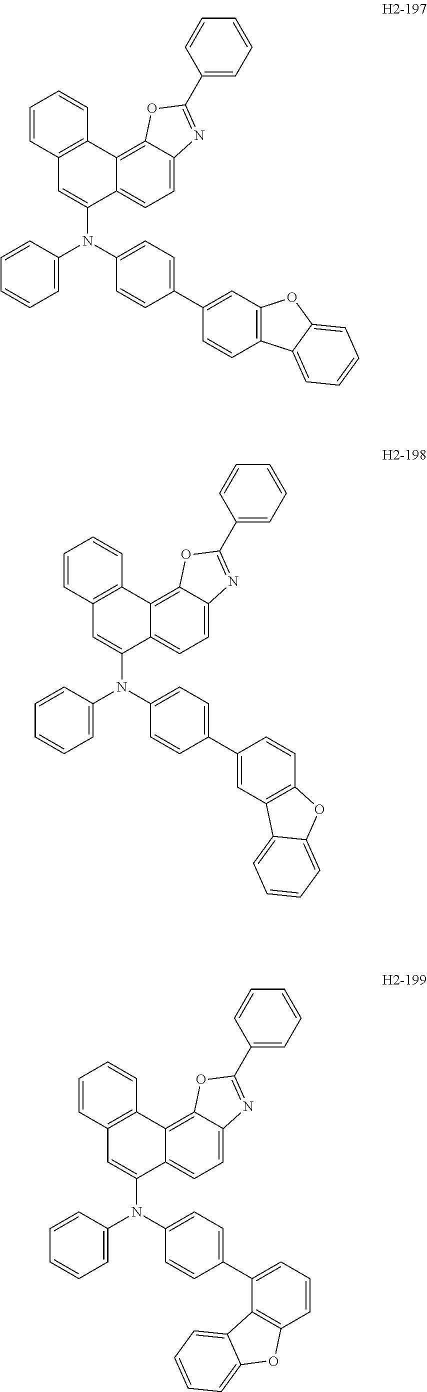 [SMILES: c1ccc(-c2nc3ccc4c(N(c5ccccc5)c5ccc(-c6ccc7c(c6)oc6ccccc67)cc5)cc5ccccc5c4c3o2)cc1 ; c1ccc(-c2nc3ccc4c(N(c5ccccc5)c5ccc(-c6ccc7oc8ccccc8c7c6)cc5)cc5ccccc5c4c3o2)cc1 ; c1ccc(-c2nc3ccc4c(N(c5ccccc5)c5ccc(-c6cccc7oc8ccccc8c67)cc5)cc5ccccc5c4c3o2)cc1]

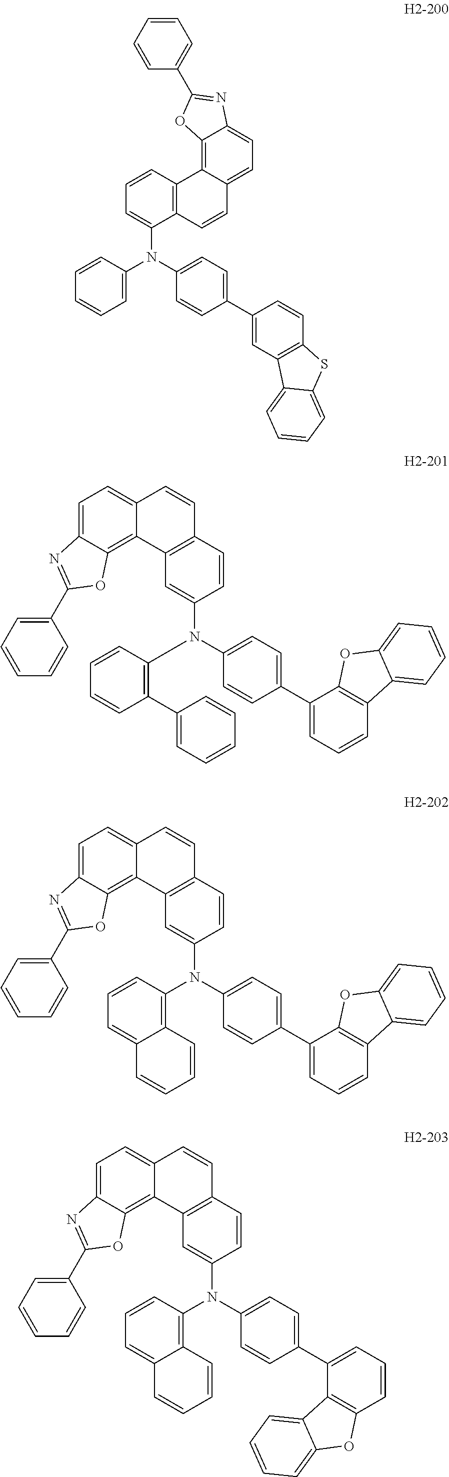 [SMILES: c1ccc(-c2nc3ccc4ccc5c(N(c6ccccc6)c6ccc(-c7ccc8sc9ccccc9c8c7)cc6)cccc5c4c3o2)cc1 ; c1ccc(-c2nc3ccc4ccc5ccc(N(c6ccc(-c7cccc8c7oc7ccccc78)cc6)c6ccccc6-c6ccccc6)cc5c4c3o2)cc1 ; c1ccc(-c2nc3ccc4ccc5ccc(N(c6ccc(-c7cccc8c7oc7ccccc78)cc6)c6cccc7ccccc67)cc5c4c3o2)cc1 ; c1ccc(-c2nc3ccc4ccc5ccc(N(c6ccc(-c7cccc8oc9ccccc9c78)cc6)c6cccc7ccccc67)cc5c4c3o2)cc1]

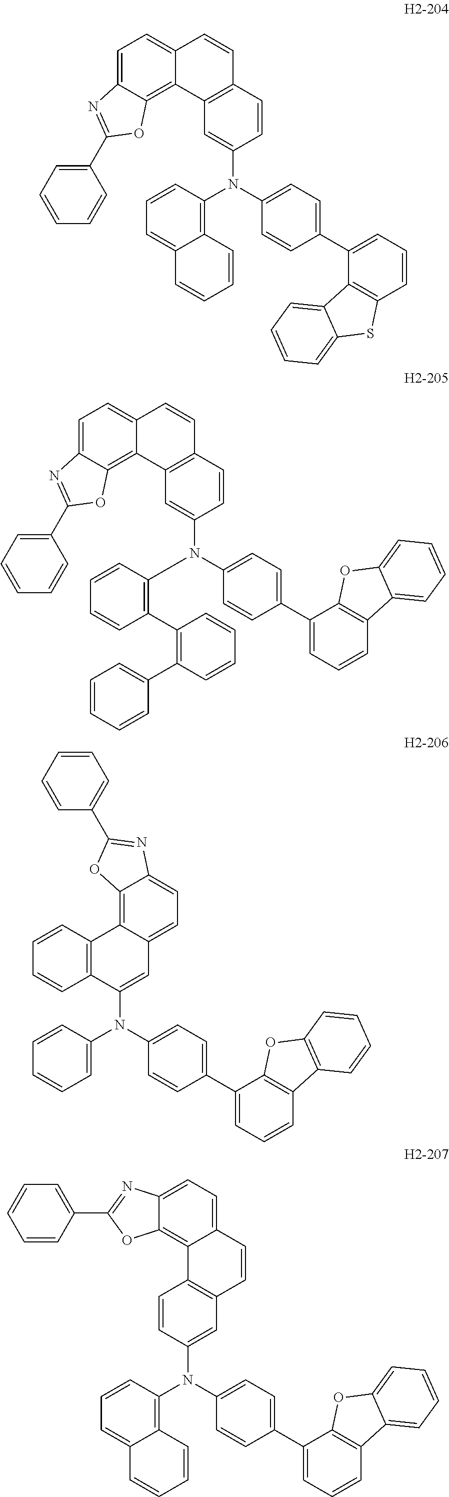

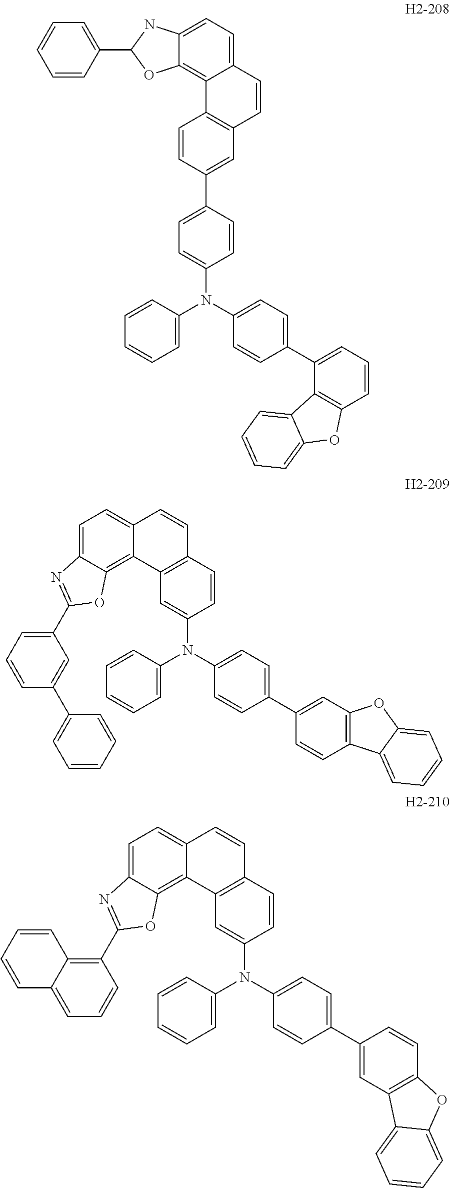

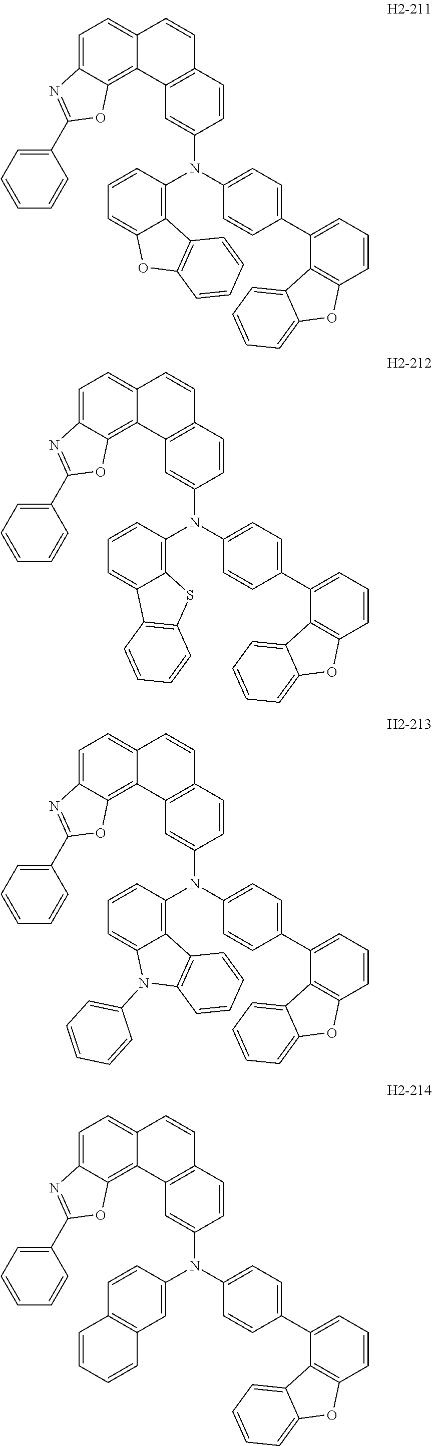 [SMILES: c1ccc(-c2nc3ccc4ccc5ccc(N(c6ccc(-c7cccc8oc9ccccc9c78)cc6)c6cccc7oc8ccccc8c67)cc5c4c3o2)cc1 ; c1ccc(-c2nc3ccc4ccc5ccc(N(c6ccc(-c7cccc8oc9ccccc9c78)cc6)c6cccc7c6sc6ccccc67)cc5c4c3o2)cc1 ; c1ccc(-c2nc3ccc4ccc5ccc(N(c6ccc(-c7cccc8oc9ccccc9c78)cc6)c6cccc7c6c6ccccc6n7-c6ccccc6)cc5c4c3o2)cc1 ; c1ccc(-c2nc3ccc4ccc5ccc(N(c6ccc(-c7cccc8oc9ccccc9c78)cc6)c6ccc7ccccc7c6)cc5c4c3o2)cc1]

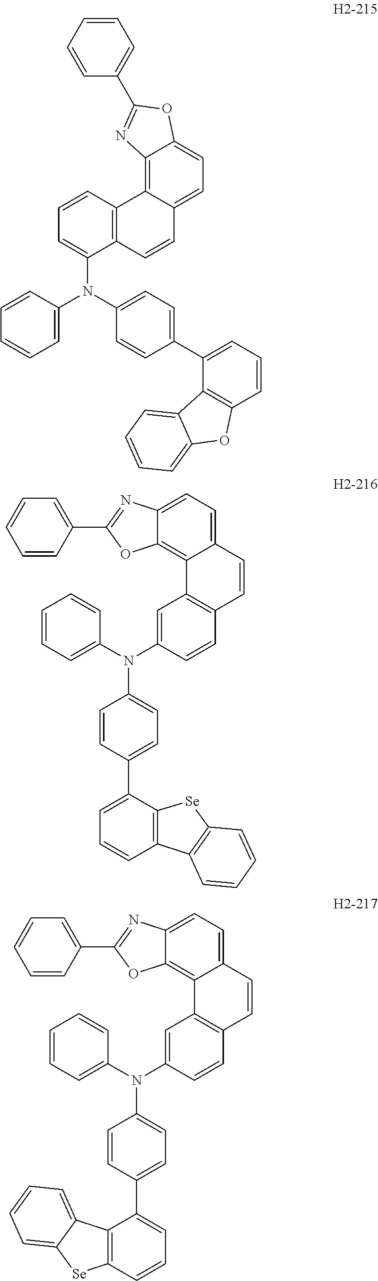

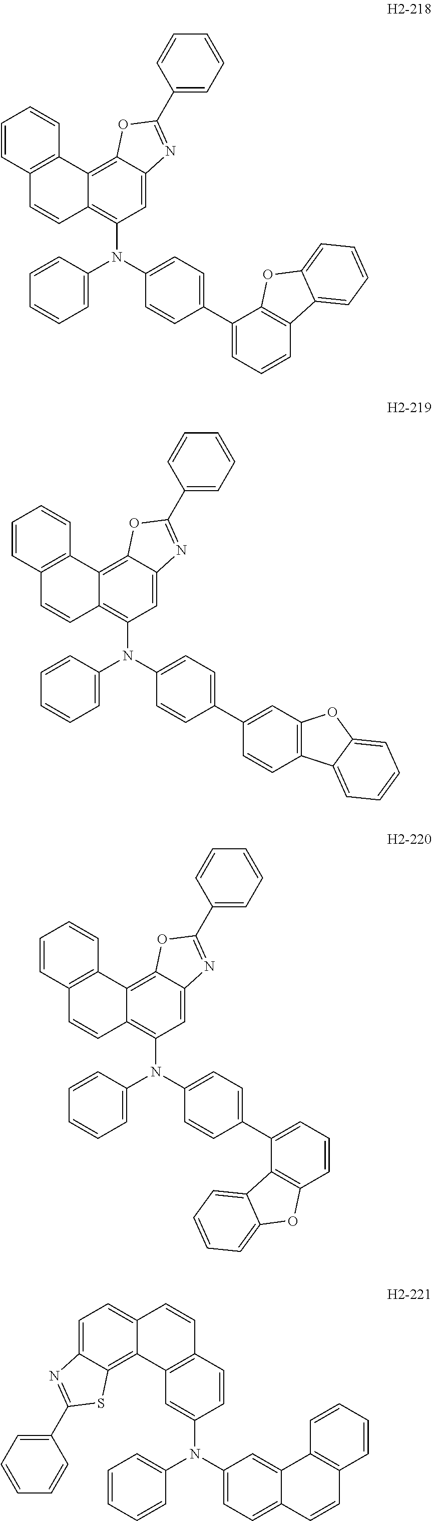 [SMILES: c1ccc(-c2nc3cc(N(c4ccccc4)c4ccc(-c5cccc6c5oc5ccccc56)cc4)c4ccc5ccccc5c4c3o2)cc1 ; c1ccc(-c2nc3cc(N(c4ccccc4)c4ccc(-c5ccc6c(c5)oc5ccccc56)cc4)c4ccc5ccccc5c4c3o2)cc1 ; c1ccc(-c2nc3cc(N(c4ccccc4)c4ccc(-c5cccc6oc7ccccc7c56)cc4)c4ccc5ccccc5c4c3o2)cc1 ; c1ccc(-c2nc3ccc4ccc5ccc(N(c6ccccc6)c6ccc7ccc8ccccc8c7c6)cc5c4c3s2)cc1]

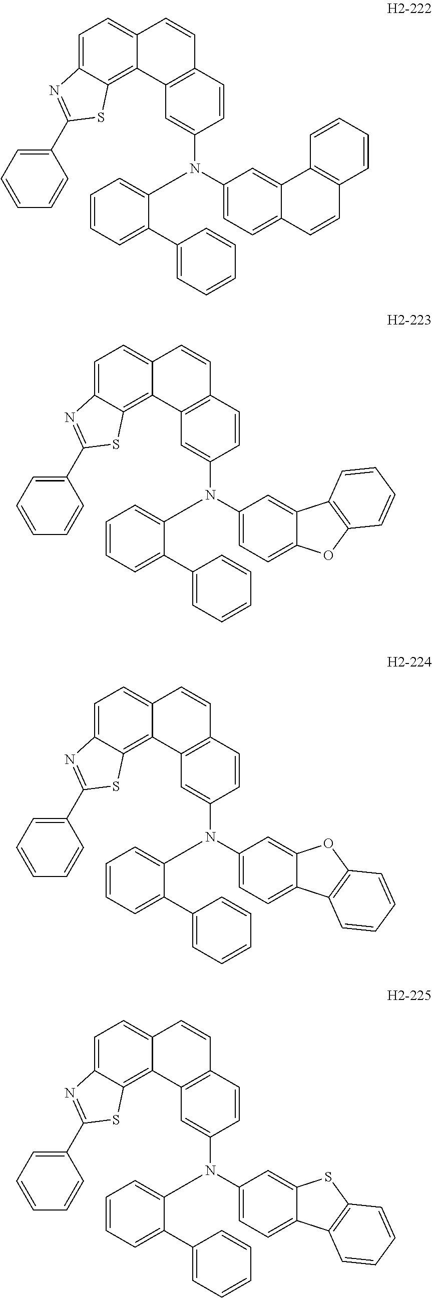

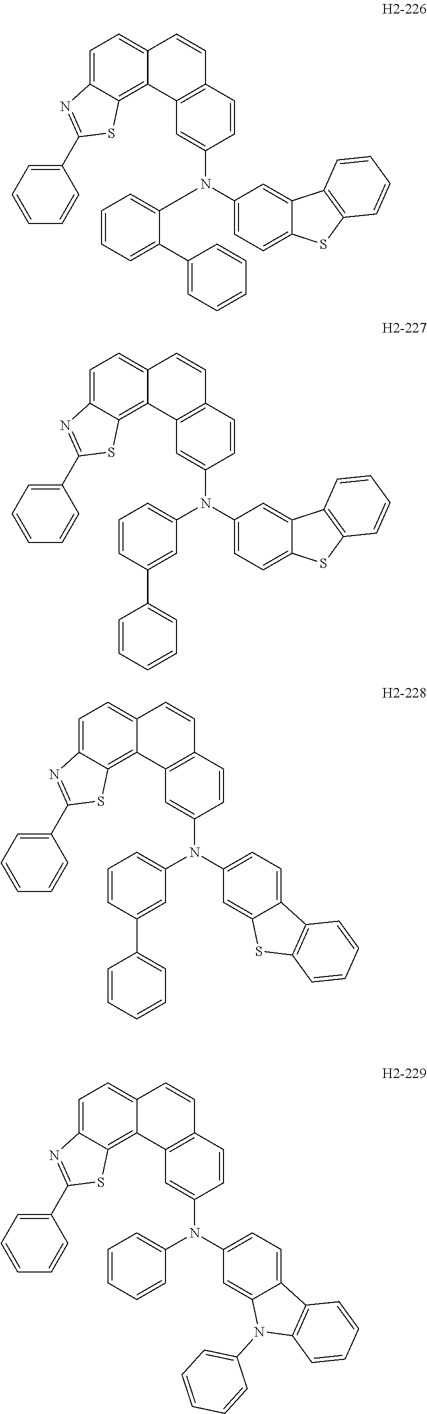

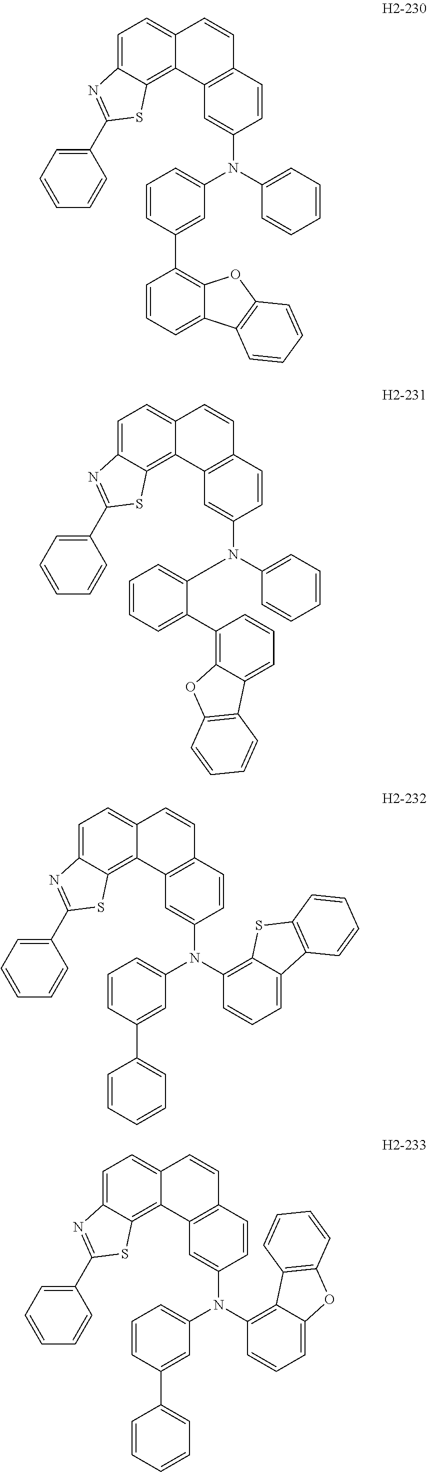

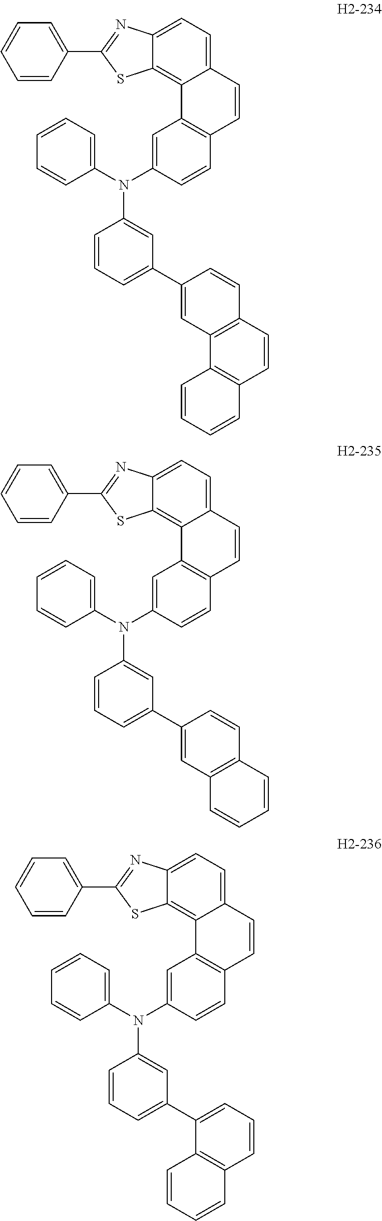

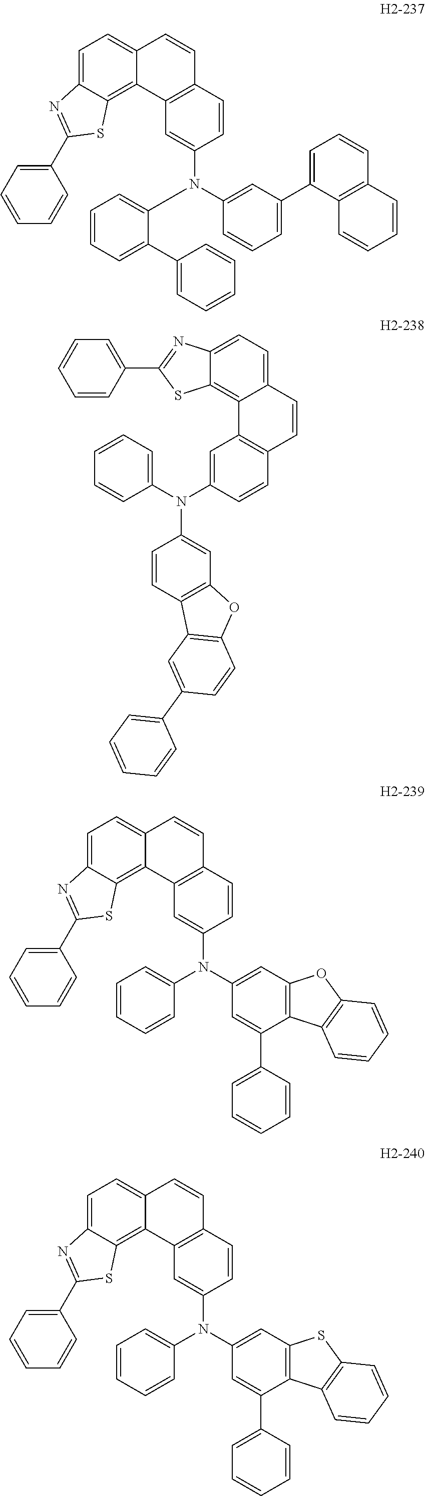

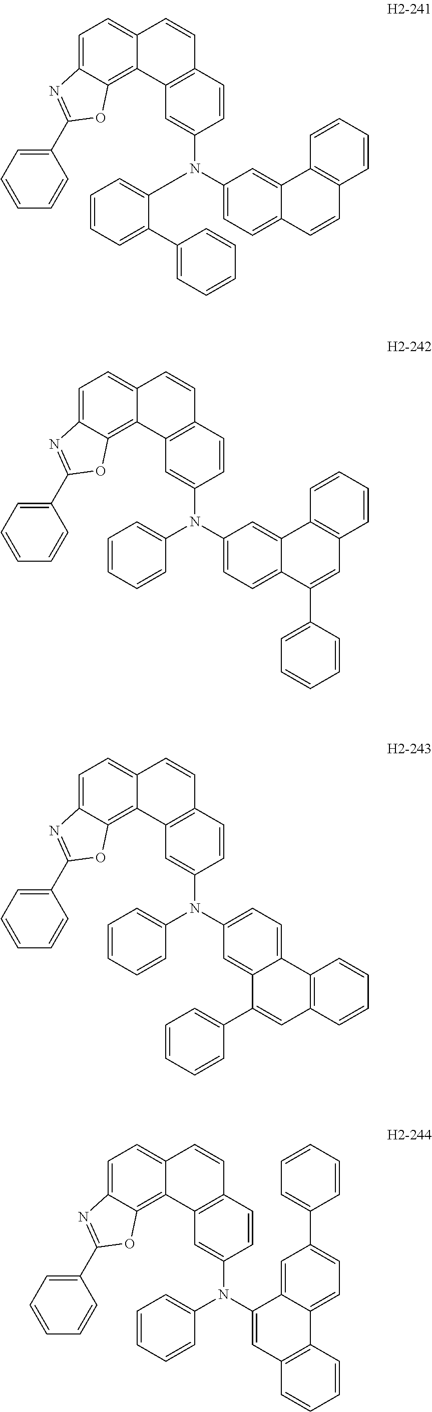 [SMILES: c1ccc(-c2nc3ccc4ccc5ccc(N(c6ccc7ccc8ccccc8c7c6)c6ccccc6-c6ccccc6)cc5c4c3o2)cc1 ; c1ccc(-c2nc3ccc4ccc5ccc(N(c6ccccc6)c6ccc7c(-c8ccccc8)cc8ccccc8c7c6)cc5c4c3o2)cc1 ; c1ccc(-c2nc3ccc4ccc5ccc(N(c6ccccc6)c6ccc7c(c6)c(-c6ccccc6)cc6ccccc67)cc5c4c3o2)cc1 ; c1ccc(-c2ccc3c(c2)c(N(c2ccccc2)c2ccc4ccc5ccc6nc(-c7ccccc7)oc6c5c4c2)cc2ccccc23)cc1]

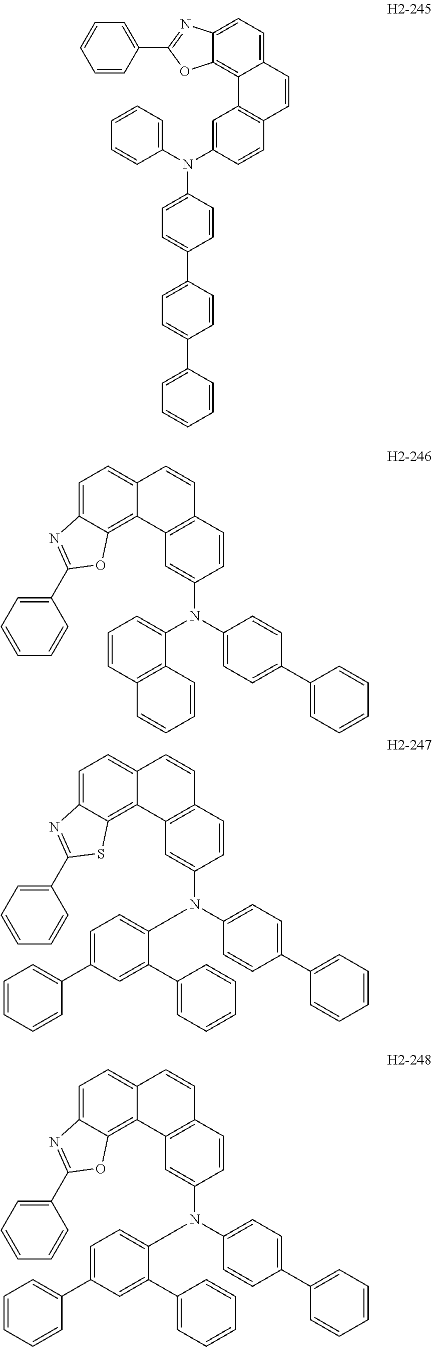

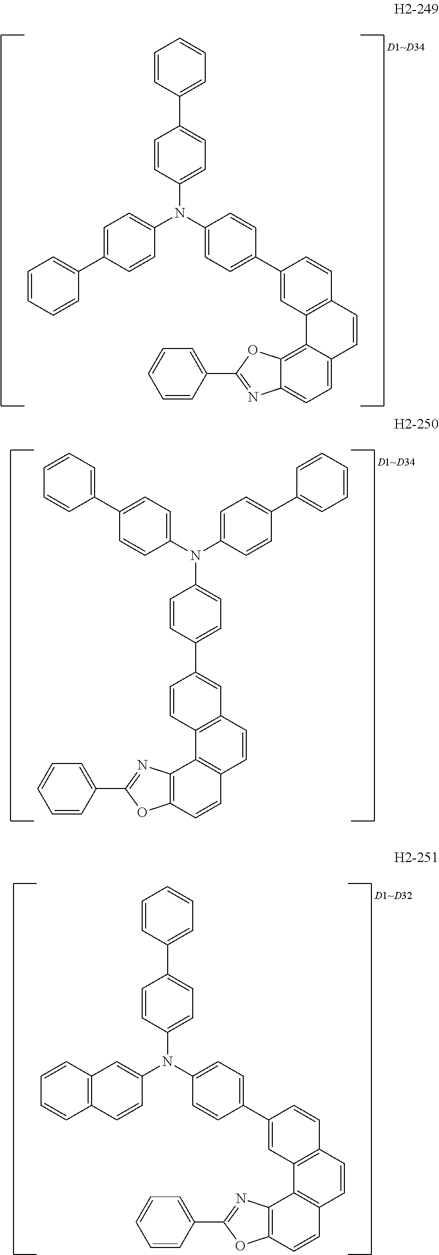

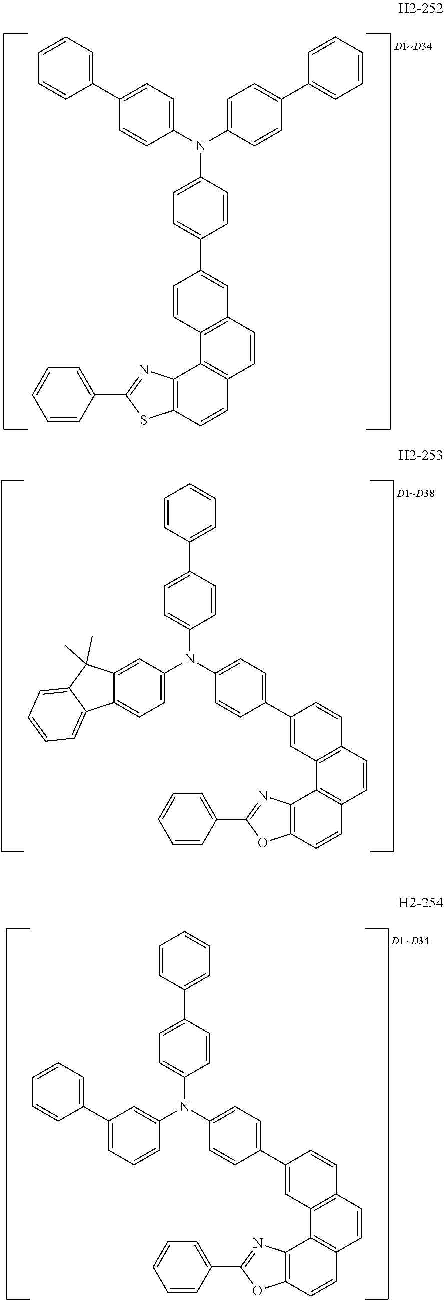

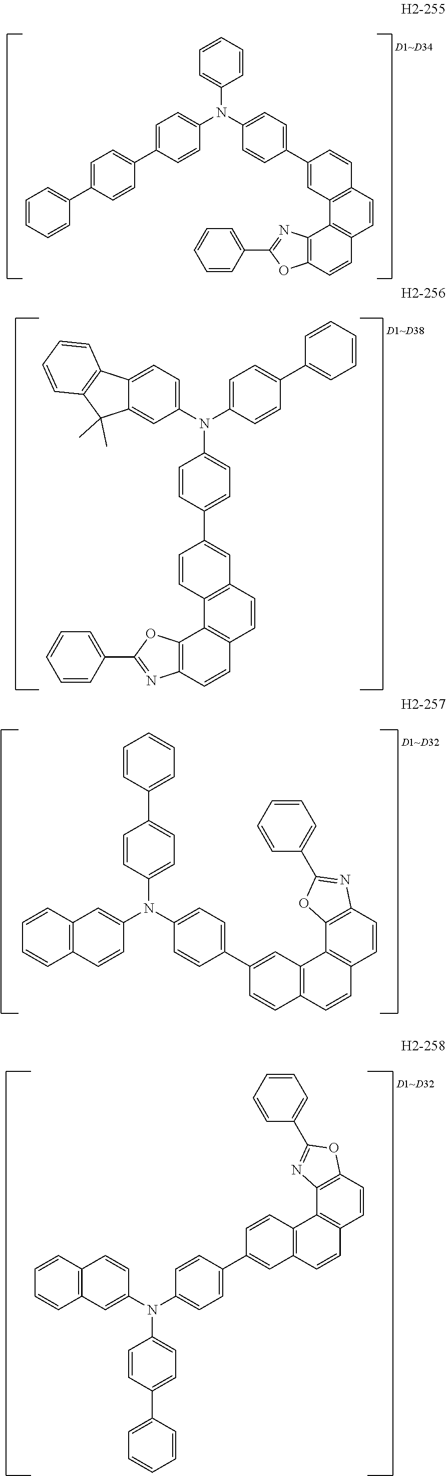

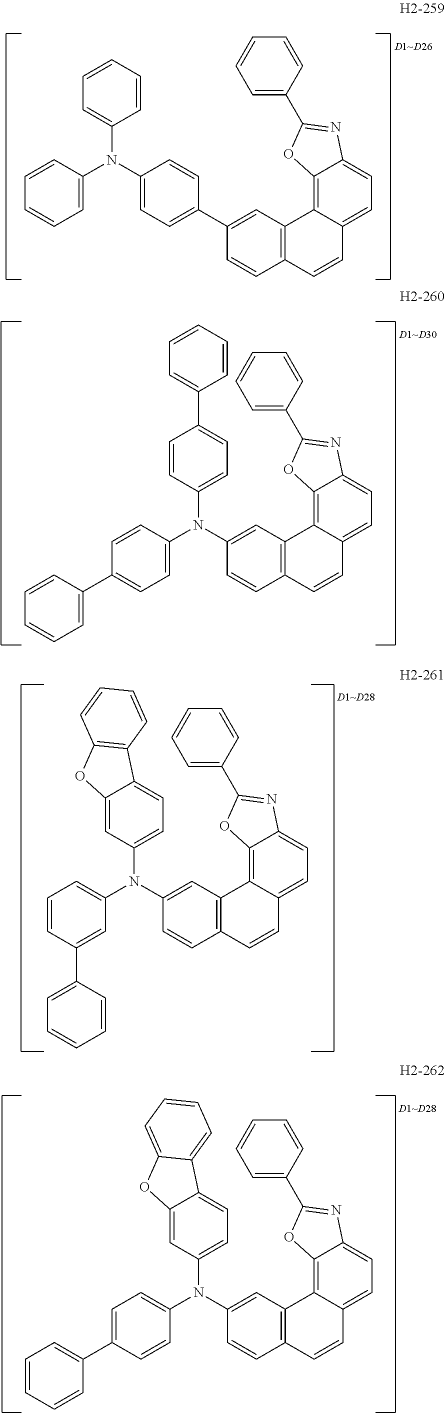

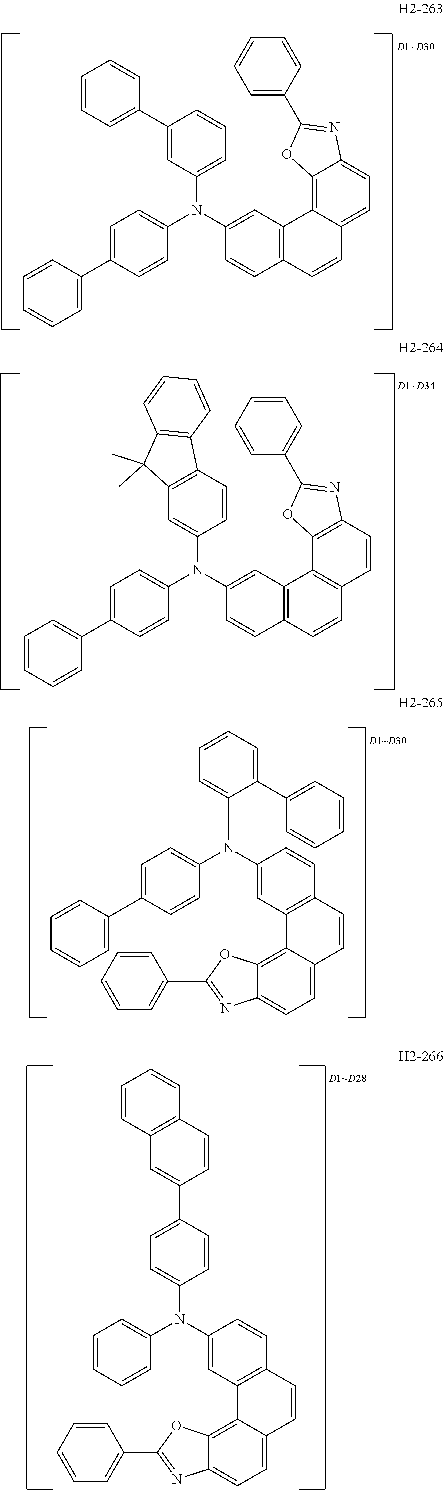

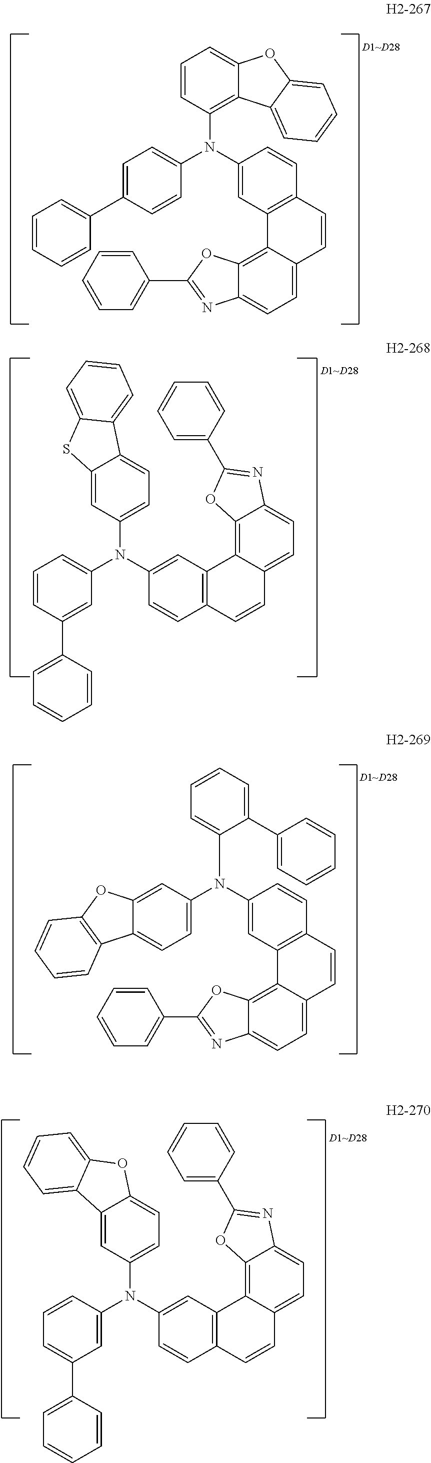

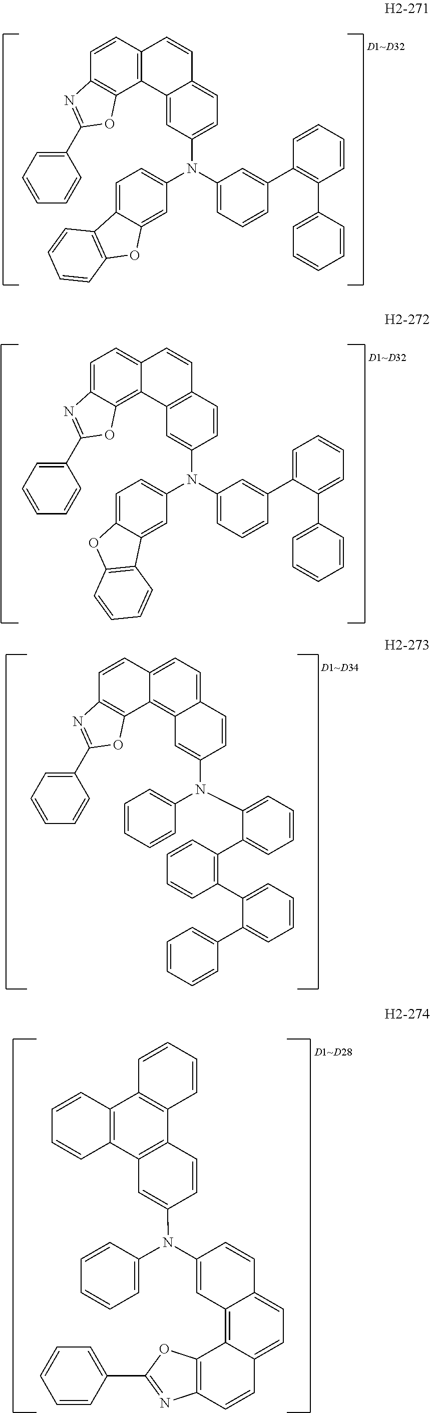

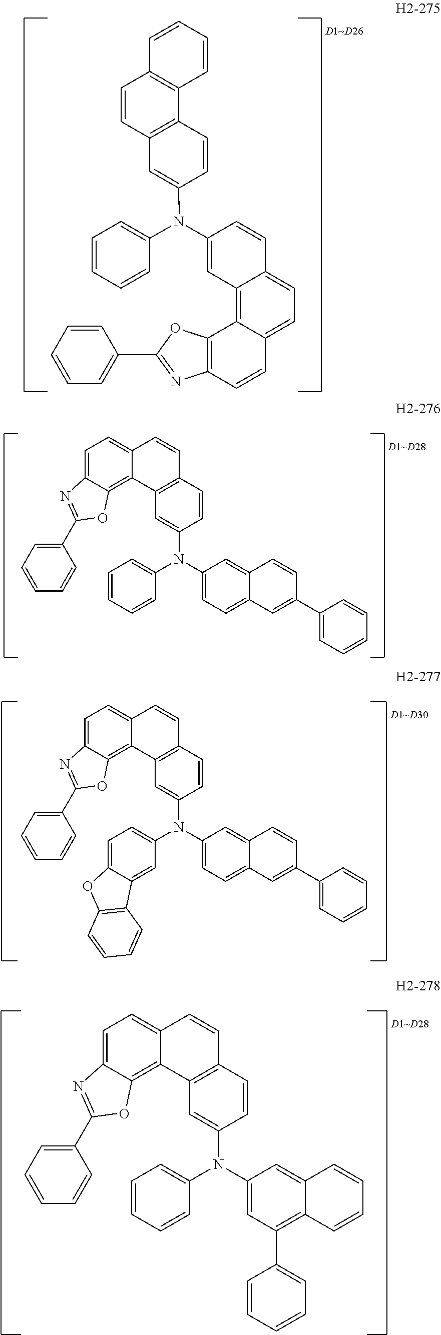

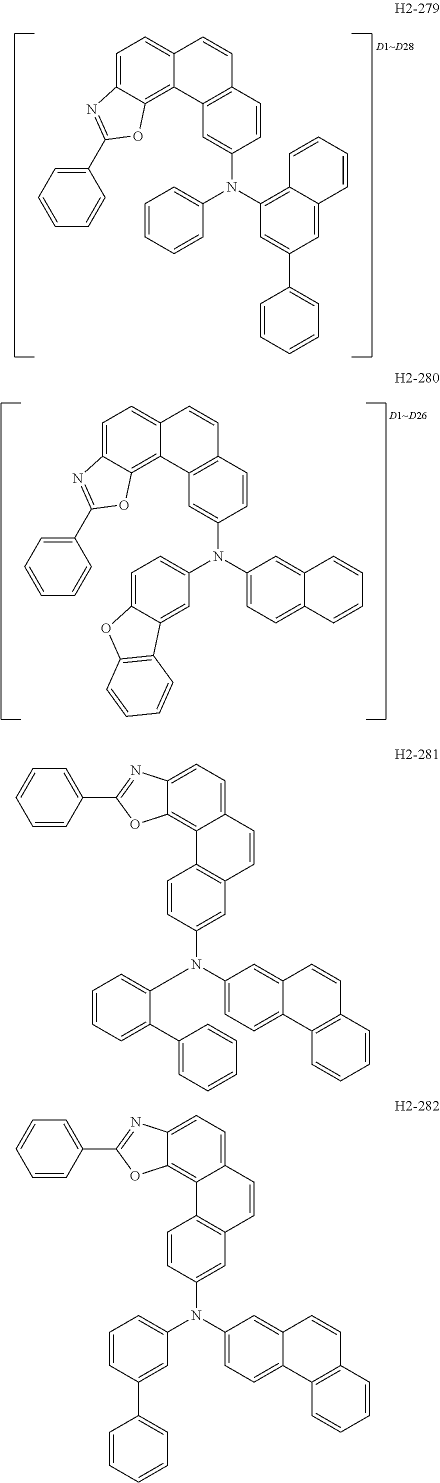

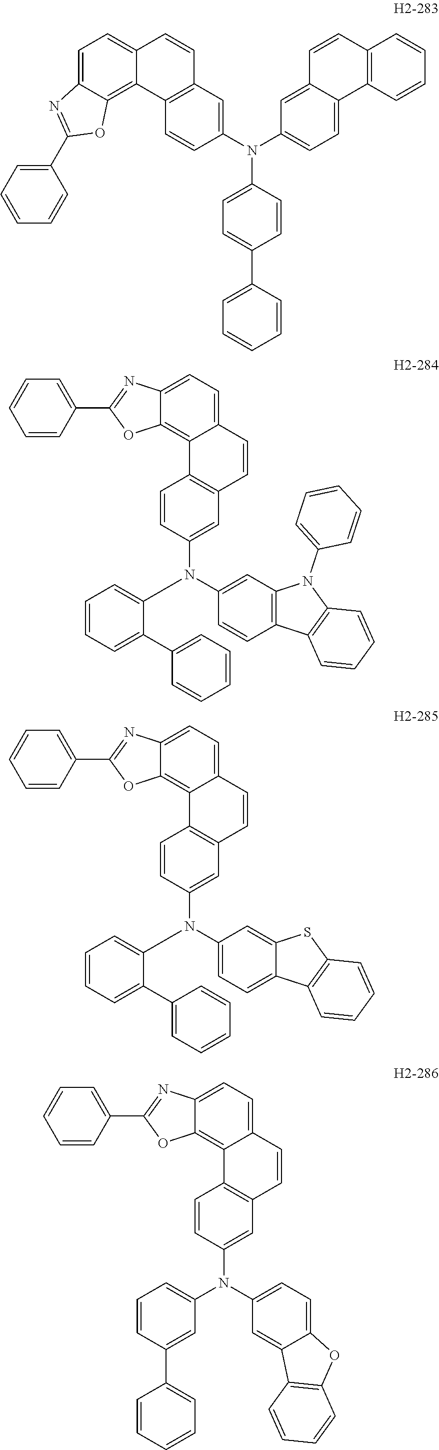

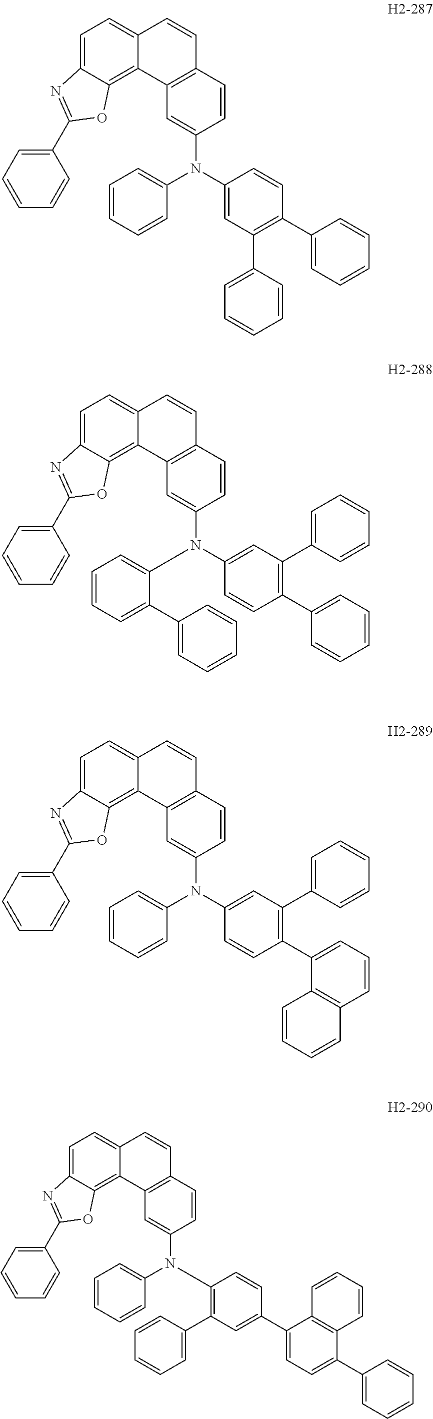

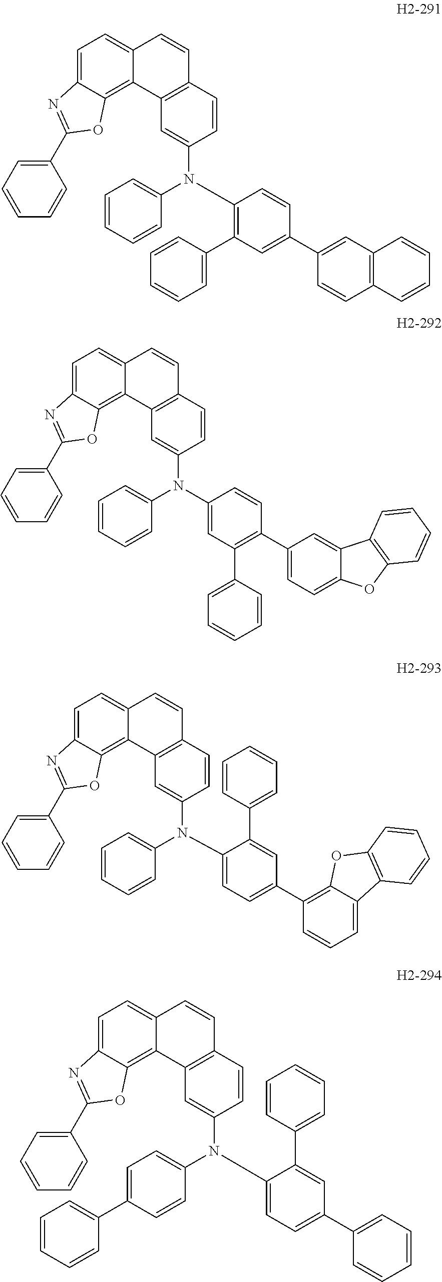 [SMILES: c1ccc(-c2nc3ccc4ccc5ccc(N(c6ccccc6)c6ccc(-c7ccc8ccccc8c7)cc6-c6ccccc6)cc5c4c3o2)cc1 ; c1ccc(-c2nc3ccc4ccc5ccc(N(c6ccccc6)c6ccc(-c7ccc8oc9ccccc9c8c7)c(-c7ccccc7)c6)cc5c4c3o2)cc1 ; c1ccc(-c2nc3ccc4ccc5ccc(N(c6ccccc6)c6ccc(-c7cccc8c7oc7ccccc78)cc6-c6ccccc6)cc5c4c3o2)cc1 ; c1ccc(-c2ccc(N(c3ccc4ccc5ccc6nc(-c7ccccc7)oc6c5c4c3)c3ccc(-c4ccccc4)cc3-c3ccccc3)cc2)cc1]

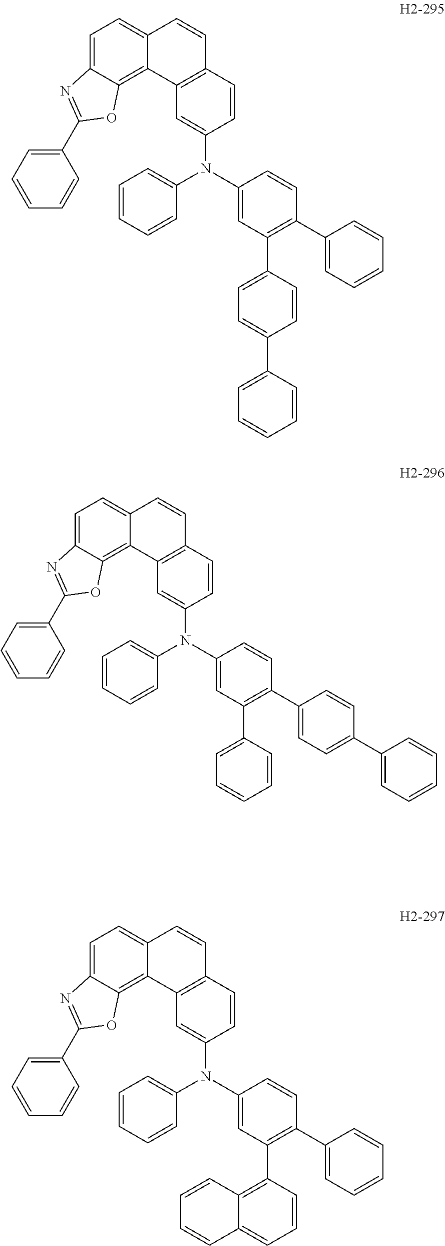 [SMILES: c1ccc(-c2ccc(-c3cc(N(c4ccccc4)c4ccc5ccc6ccc7nc(-c8ccccc8)oc7c6c5c4)ccc3-c3ccccc3)cc2)cc1 ; c1ccc(-c2ccc(-c3ccc(N(c4ccccc4)c4ccc5ccc6ccc7nc(-c8ccccc8)oc7c6c5c4)cc3-c3ccccc3)cc2)cc1 ; c1ccc(-c2nc3ccc4ccc5ccc(N(c6ccccc6)c6ccc(-c7ccccc7)c(-c7cccc8ccccc78)c6)cc5c4c3o2)cc1]

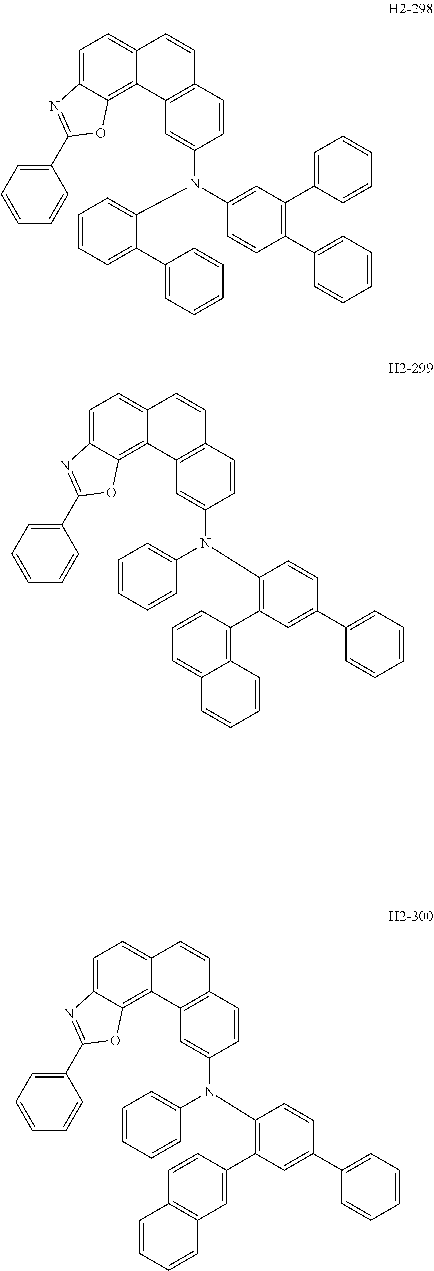 [SMILES: c1ccc(-c2nc3ccc4ccc5ccc(N(c6ccc(-c7ccccc7)c(-c7ccccc7)c6)c6ccccc6-c6ccccc6)cc5c4c3o2)cc1 ; c1ccc(-c2ccc(N(c3ccccc3)c3ccc4ccc5ccc6nc(-c7ccccc7)oc6c5c4c3)c(-c3cccc4ccccc34)c2)cc1 ; c1ccc(-c2ccc(N(c3ccccc3)c3ccc4ccc5ccc6nc(-c7ccccc7)oc6c5c4c3)c(-c3ccc4ccccc4c3)c2)cc1]

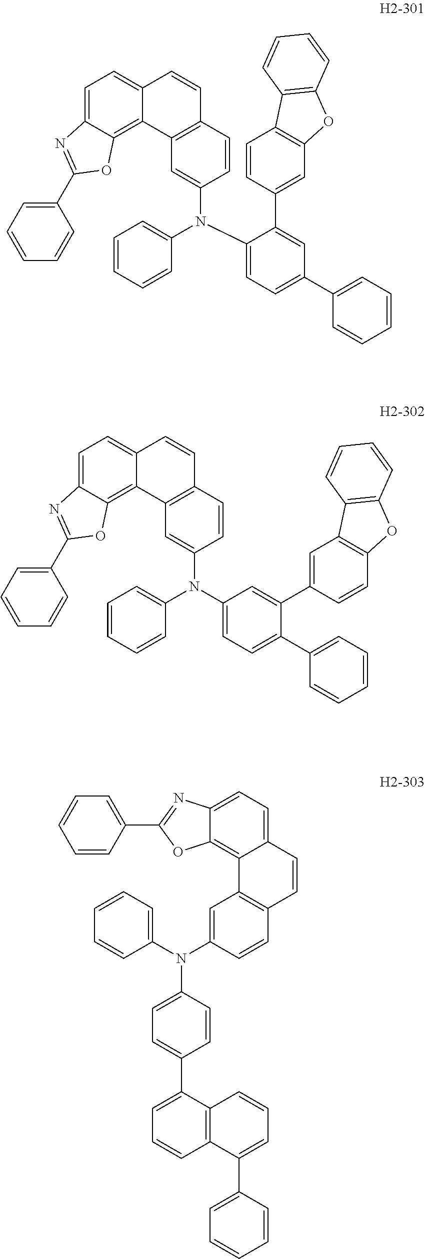 [SMILES: c1ccc(-c2ccc(N(c3ccccc3)c3ccc4ccc5ccc6nc(-c7ccccc7)oc6c5c4c3)c(-c3ccc4c(c3)oc3ccccc34)c2)cc1 ; c1ccc(-c2nc3ccc4ccc5ccc(N(c6ccccc6)c6ccc(-c7ccccc7)c(-c7ccc8oc9ccccc9c8c7)c6)cc5c4c3o2)cc1 ; c1ccc(-c2nc3ccc4ccc5ccc(N(c6ccccc6)c6ccc(-c7cccc8c(-c9ccccc9)cccc78)cc6)cc5c4c3o2)cc1]

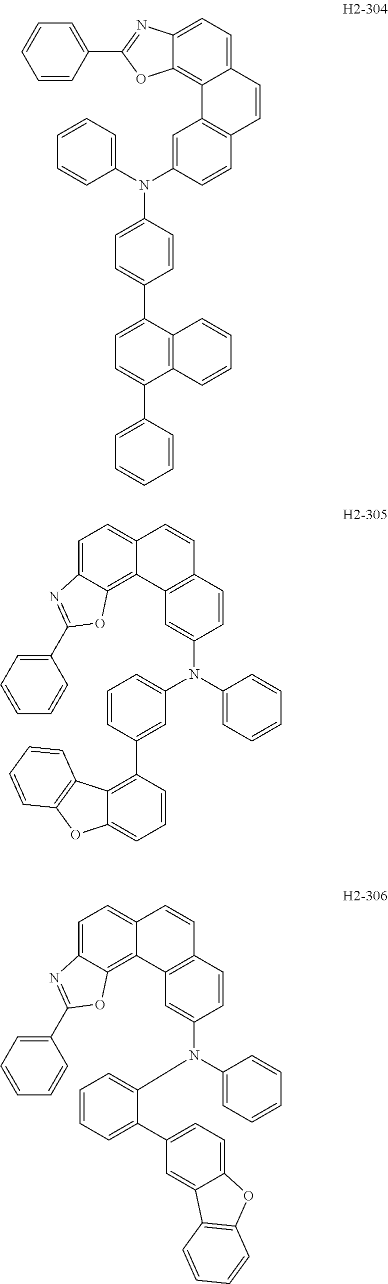

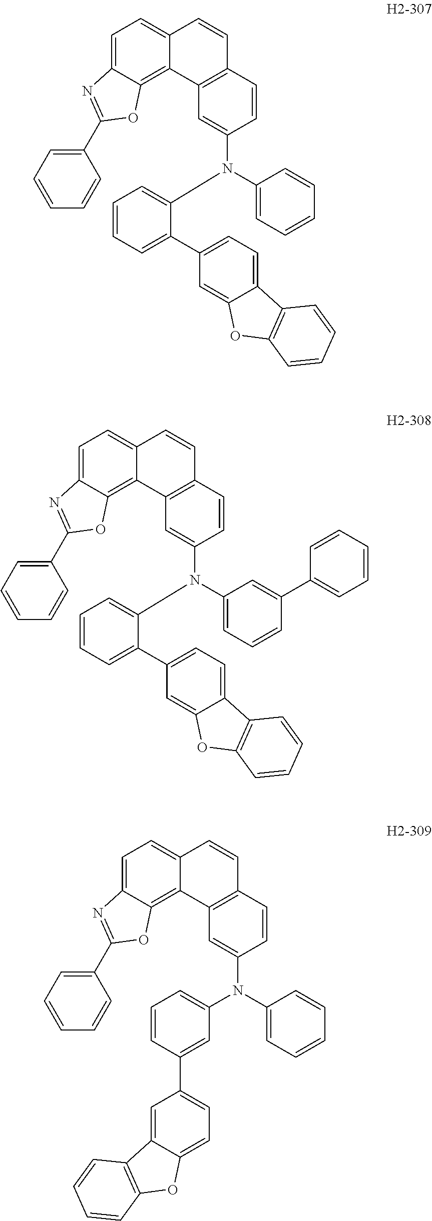 [SMILES: c1ccc(-c2nc3ccc4ccc5ccc(N(c6ccccc6)c6ccccc6-c6ccc7c(c6)oc6ccccc67)cc5c4c3o2)cc1 ; c1ccc(-c2cccc(N(c3ccc4ccc5ccc6nc(-c7ccccc7)oc6c5c4c3)c3ccccc3-c3ccc4c(c3)oc3ccccc34)c2)cc1 ; c1ccc(-c2nc3ccc4ccc5ccc(N(c6ccccc6)c6cccc(-c7ccc8oc9ccccc9c8c7)c6)cc5c4c3o2)cc1]

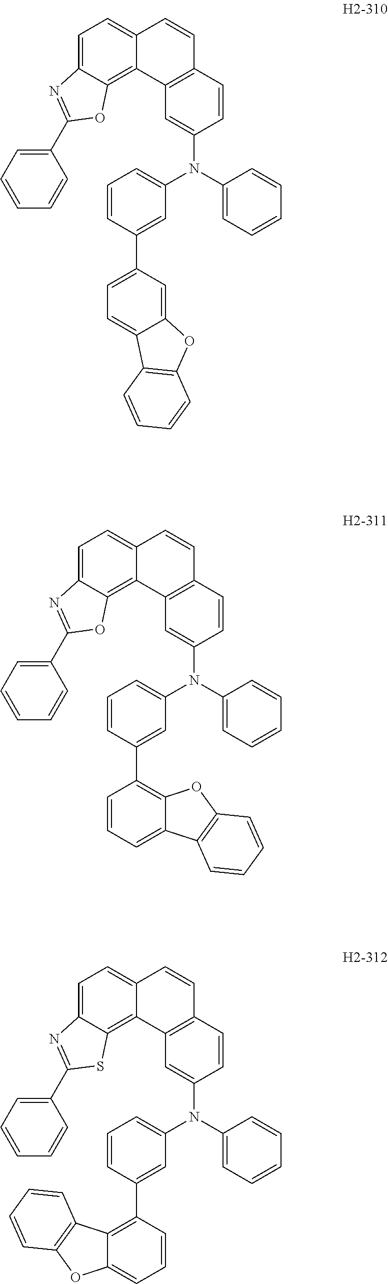 [SMILES: c1ccc(-c2nc3ccc4ccc5ccc(N(c6ccccc6)c6cccc(-c7ccc8c(c7)oc7ccccc78)c6)cc5c4c3o2)cc1 ; c1ccc(-c2nc3ccc4ccc5ccc(N(c6ccccc6)c6cccc(-c7cccc8c7oc7ccccc78)c6)cc5c4c3o2)cc1 ; c1ccc(-c2nc3ccc4ccc5ccc(N(c6ccccc6)c6cccc(-c7cccc8oc9ccccc9c78)c6)cc5c4c3s2)cc1]

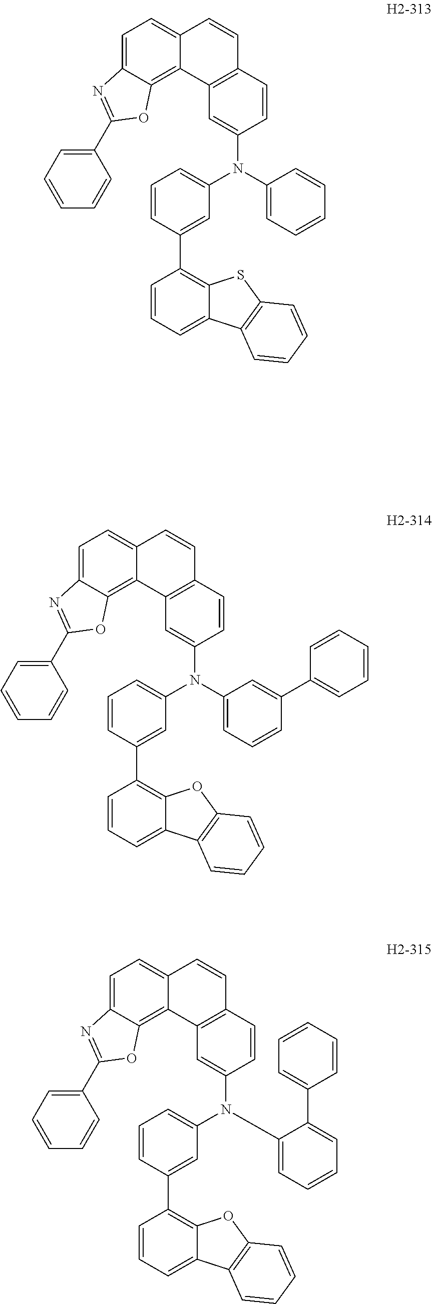 [SMILES: c1ccc(-c2nc3ccc4ccc5ccc(N(c6ccccc6)c6cccc(-c7cccc8c7sc7ccccc78)c6)cc5c4c3o2)cc1 ; c1ccc(-c2cccc(N(c3cccc(-c4cccc5c4oc4ccccc45)c3)c3ccc4ccc5ccc6nc(-c7ccccc7)oc6c5c4c3)c2)cc1 ; c1ccc(-c2nc3ccc4ccc5ccc(N(c6cccc(-c7cccc8c7oc7ccccc78)c6)c6ccccc6-c6ccccc6)cc5c4c3o2)cc1]

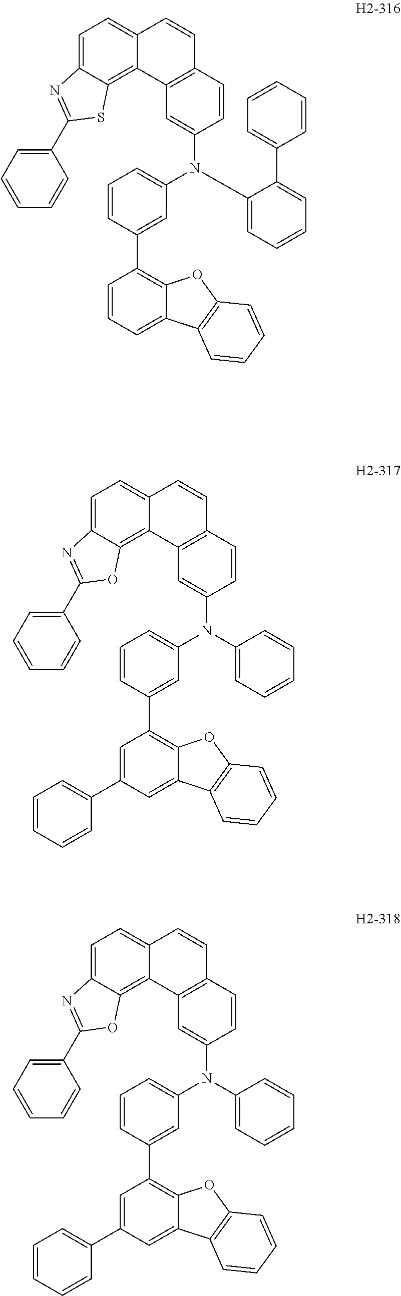 [SMILES: c1ccc(-c2nc3ccc4ccc5ccc(N(c6cccc(-c7cccc8c7oc7ccccc78)c6)c6ccccc6-c6ccccc6)cc5c4c3s2)cc1 ; c1ccc(-c2cc(-c3cccc(N(c4ccccc4)c4ccc5ccc6ccc7nc(-c8ccccc8)oc7c6c5c4)c3)c3oc4ccccc4c3c2)cc1 ; c1ccc(-c2cc(-c3cccc(N(c4ccccc4)c4ccc5ccc6ccc7nc(-c8ccccc8)oc7c6c5c4)c3)c3oc4ccccc4c3c2)cc1]

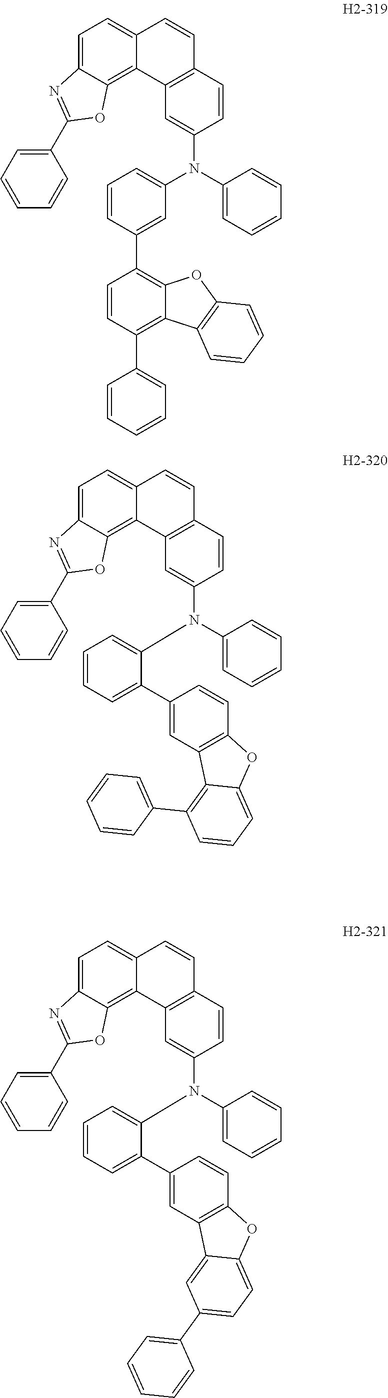

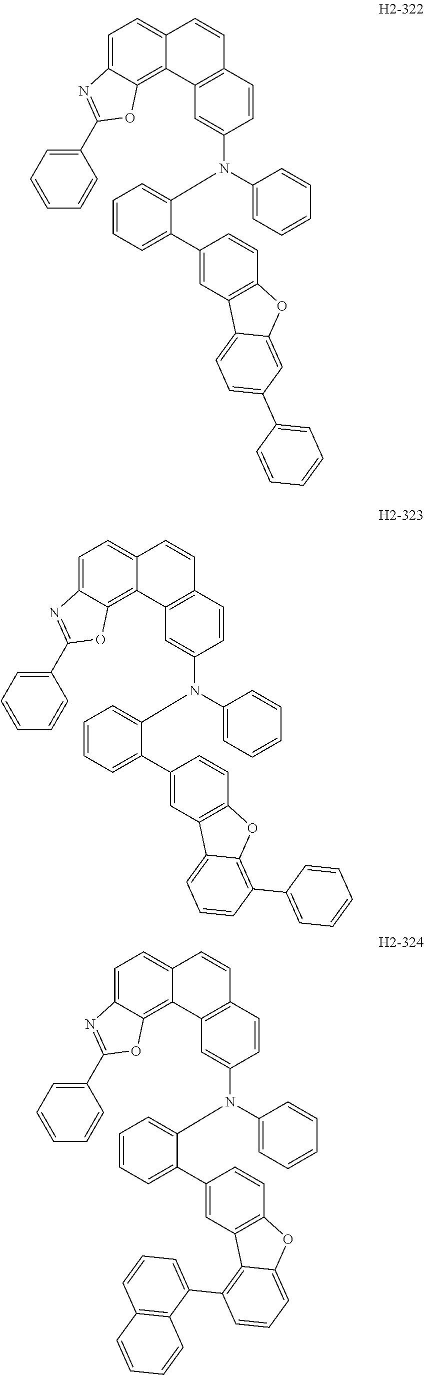

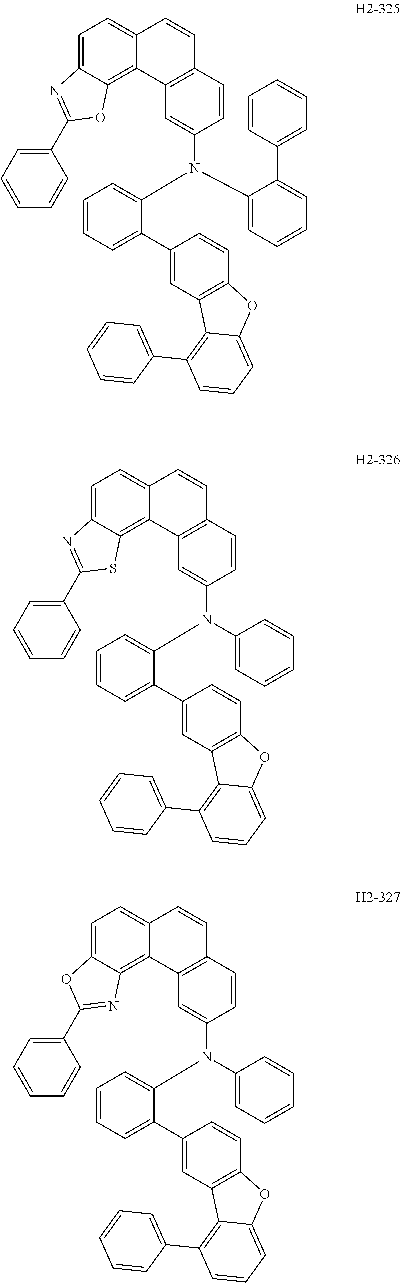 [SMILES: c1ccc(-c2nc3ccc4ccc5ccc(N(c6ccccc6-c6ccccc6)c6ccccc6-c6ccc7oc8cccc(-c9ccccc9)c8c7c6)cc5c4c3o2)cc1 ; c1ccc(-c2nc3ccc4ccc5ccc(N(c6ccccc6)c6ccccc6-c6ccc7oc8cccc(-c9ccccc9)c8c7c6)cc5c4c3s2)cc1 ; c1ccc(-c2nc3c(ccc4ccc5ccc(N(c6ccccc6)c6ccccc6-c6ccc7oc8cccc(-c9ccccc9)c8c7c6)cc5c43)o2)cc1]

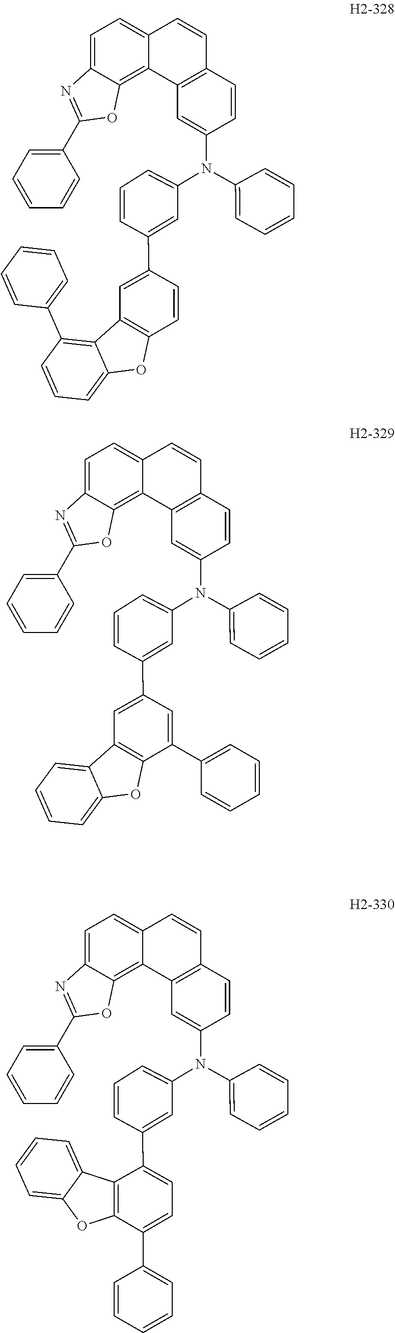 [SMILES: c1ccc(-c2nc3ccc4ccc5ccc(N(c6ccccc6)c6cccc(-c7ccc8oc9cccc(-c%10ccccc%10)c9c8c7)c6)cc5c4c3o2)cc1 ; c1ccc(-c2nc3ccc4ccc5ccc(N(c6ccccc6)c6cccc(-c7cc(-c8ccccc8)c8oc9ccccc9c8c7)c6)cc5c4c3o2)cc1 ; c1ccc(-c2nc3ccc4ccc5ccc(N(c6ccccc6)c6cccc(-c7ccc(-c8ccccc8)c8oc9ccccc9c78)c6)cc5c4c3o2)cc1]

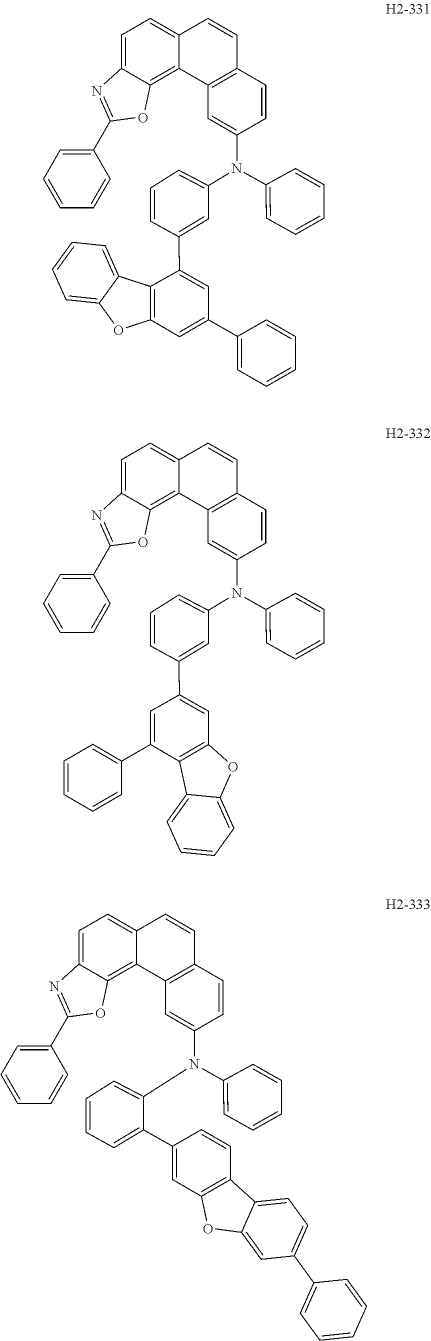 [SMILES: c1ccc(-c2cc(-c3cccc(N(c4ccccc4)c4ccc5ccc6ccc7nc(-c8ccccc8)oc7c6c5c4)c3)c3c(c2)oc2ccccc23)cc1 ; c1ccc(-c2nc3ccc4ccc5ccc(N(c6ccccc6)c6cccc(-c7cc(-c8ccccc8)c8c(c7)oc7ccccc78)c6)cc5c4c3o2)cc1 ; c1ccc(-c2ccc3c(c2)oc2cc(-c4ccccc4N(c4ccccc4)c4ccc5ccc6ccc7nc(-c8ccccc8)oc7c6c5c4)ccc23)cc1]

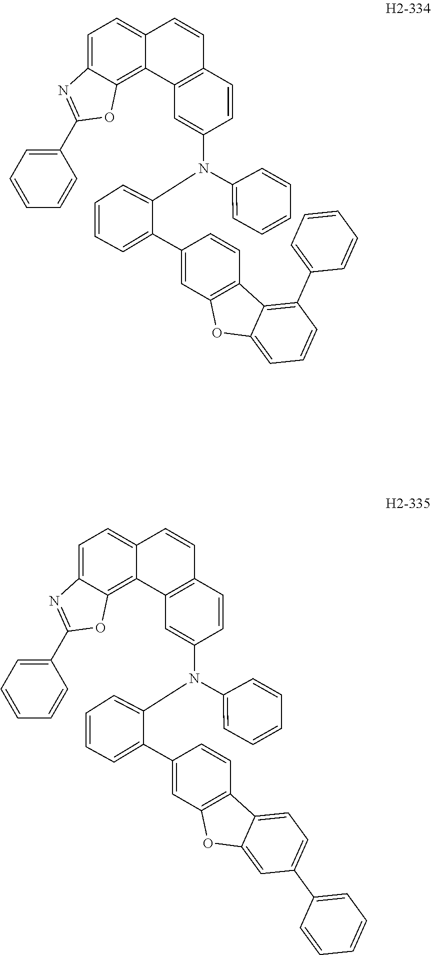 [SMILES: c1ccc(-c2nc3ccc4ccc5ccc(N(c6ccccc6)c6ccccc6-c6ccc7c(c6)oc6cccc(-c8ccccc8)c67)cc5c4c3o2)cc1 ; c1ccc(-c2ccc3c(c2)oc2cc(-c4ccccc4N(c4ccccc4)c4ccc5ccc6ccc7nc(-c8ccccc8)oc7c6c5c4)ccc23)cc1]

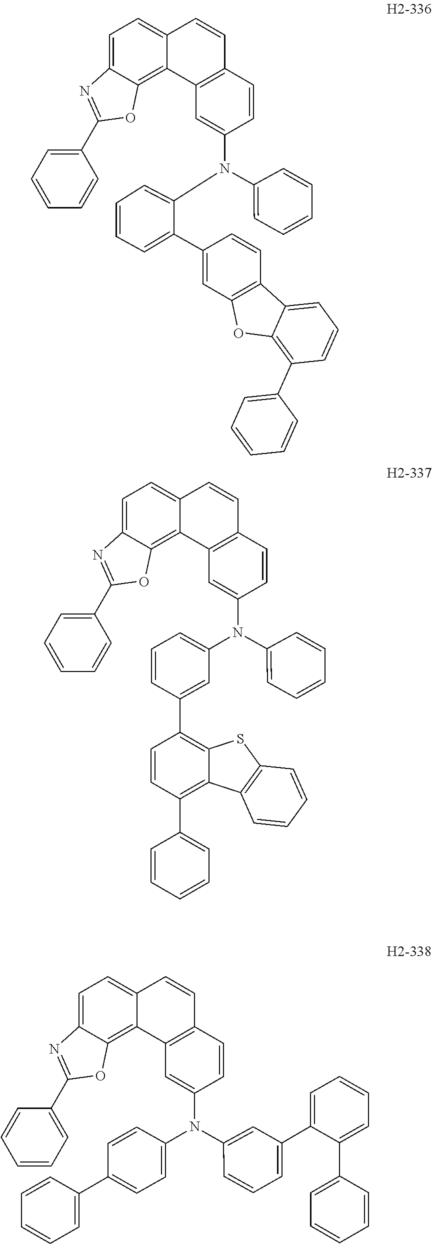

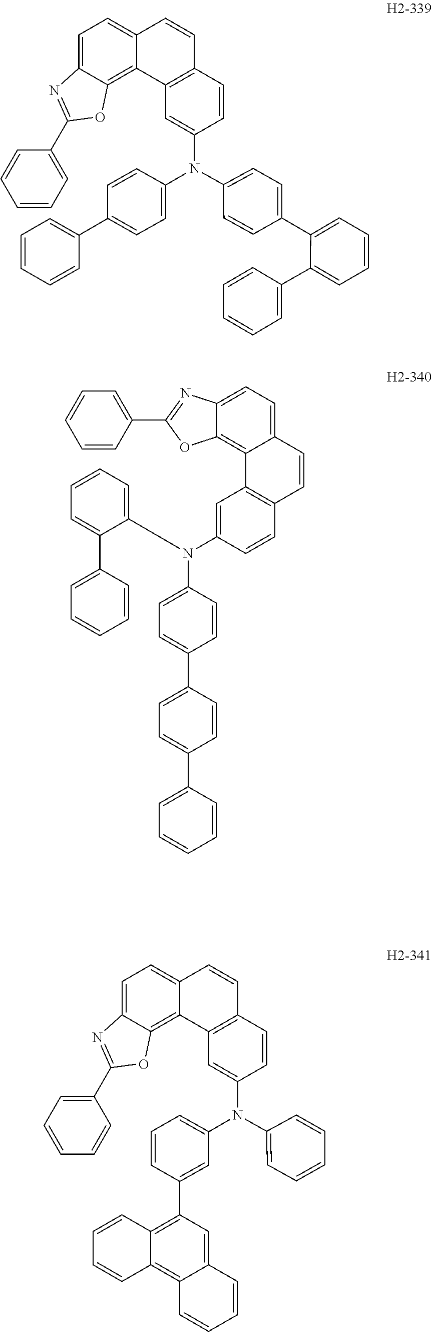 [SMILES: c1ccc(-c2ccc(N(c3ccc(-c4ccccc4-c4ccccc4)cc3)c3ccc4ccc5ccc6nc(-c7ccccc7)oc6c5c4c3)cc2)cc1 ; c1ccc(-c2ccc(-c3ccc(N(c4ccc5ccc6ccc7nc(-c8ccccc8)oc7c6c5c4)c4ccccc4-c4ccccc4)cc3)cc2)cc1 ; c1ccc(-c2nc3ccc4ccc5ccc(N(c6ccccc6)c6cccc(-c7cc8ccccc8c8ccccc78)c6)cc5c4c3o2)cc1]

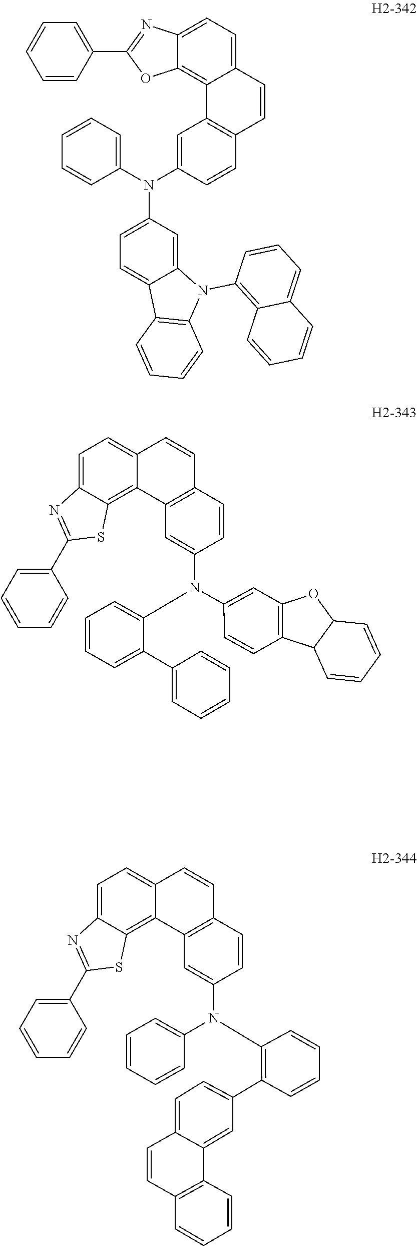 [SMILES: c1ccc(-c2nc3ccc4ccc5ccc(N(c6ccccc6)c6ccc7c8ccccc8n(-c8cccc9ccccc89)c7c6)cc5c4c3o2)cc1 ; C1=CC2Oc3cc(N(c4ccc5ccc6ccc7nc(-c8ccccc8)sc7c6c5c4)c4ccccc4-c4ccccc4)ccc3C2C=C1 ; c1ccc(-c2nc3ccc4ccc5ccc(N(c6ccccc6)c6ccccc6-c6ccc7ccc8ccccc8c7c6)cc5c4c3s2)cc1]

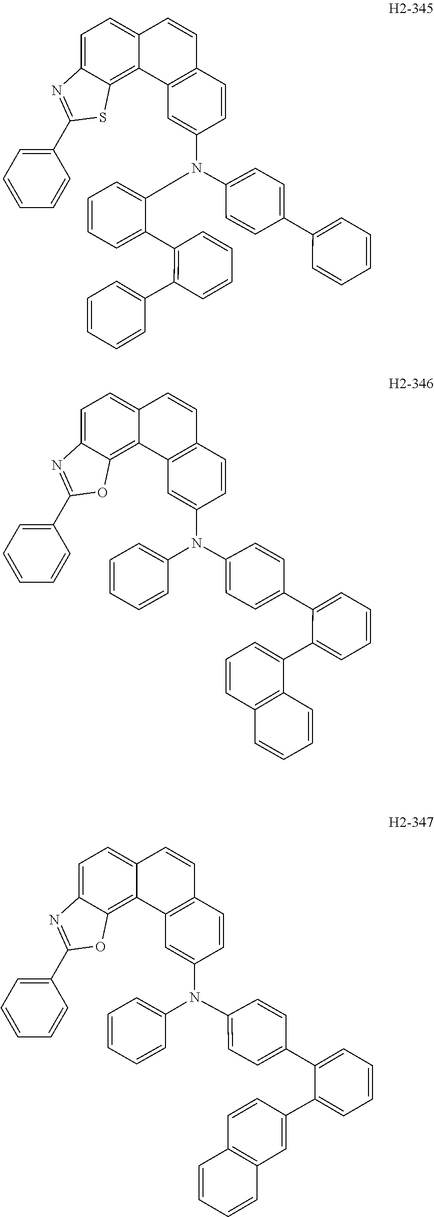

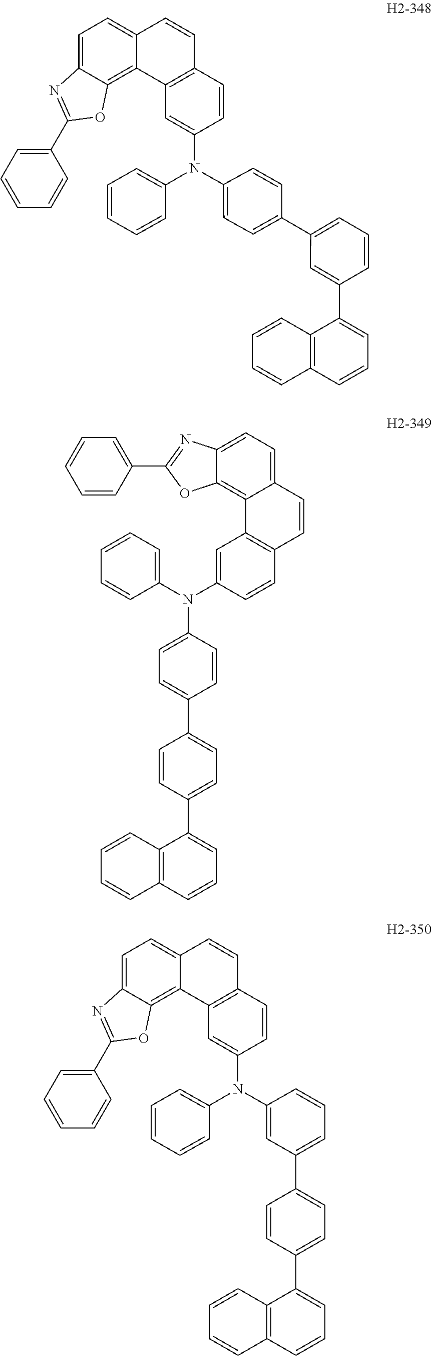

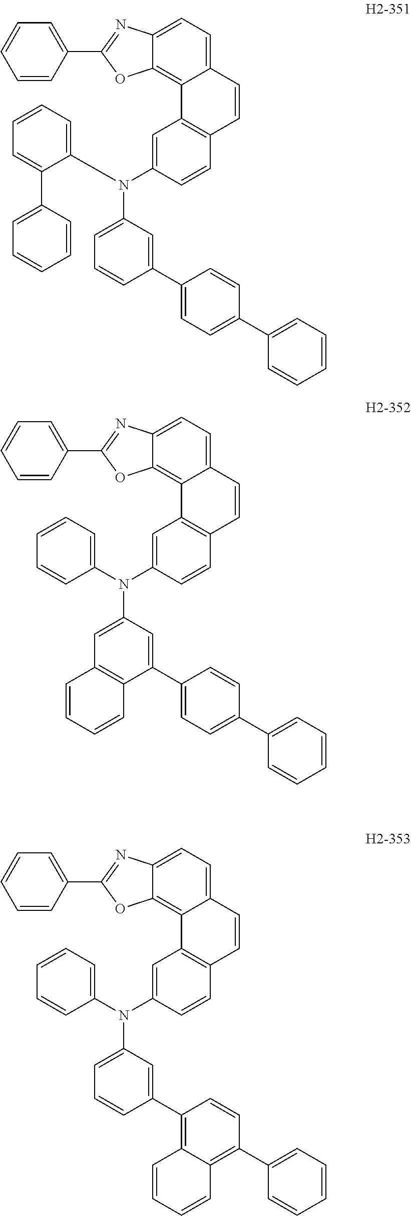 [SMILES: c1ccc(-c2ccc(-c3cccc(N(c4ccc5ccc6ccc7nc(-c8ccccc8)oc7c6c5c4)c4ccccc4-c4ccccc4)c3)cc2)cc1 ; c1ccc(-c2ccc(-c3cc(N(c4ccccc4)c4ccc5ccc6ccc7nc(-c8ccccc8)oc7c6c5c4)cc4ccccc34)cc2)cc1 ; c1ccc(-c2nc3ccc4ccc5ccc(N(c6ccccc6)c6cccc(-c7ccc(-c8ccccc8)c8ccccc78)c6)cc5c4c3o2)cc1]

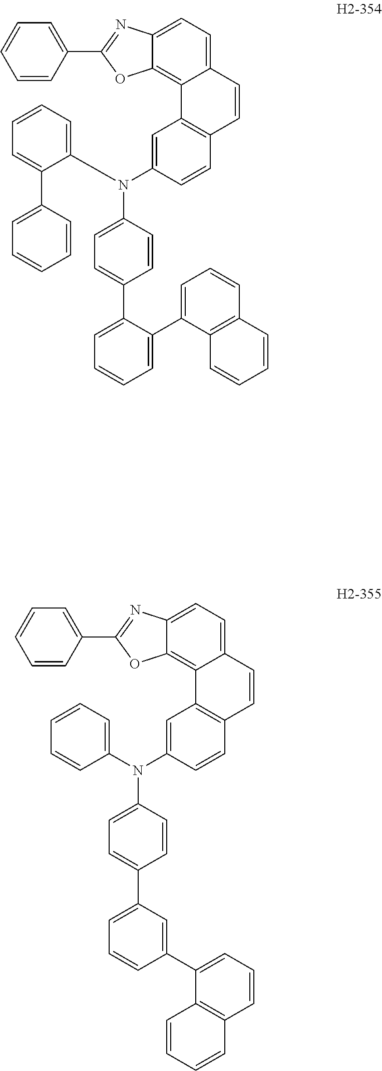 [SMILES: c1ccc(-c2nc3ccc4ccc5ccc(N(c6ccc(-c7ccccc7-c7cccc8ccccc78)cc6)c6ccccc6-c6ccccc6)cc5c4c3o2)cc1 ; c1ccc(-c2nc3ccc4ccc5ccc(N(c6ccccc6)c6ccc(-c7cccc(-c8cccc9ccccc89)c7)cc6)cc5c4c3o2)cc1]

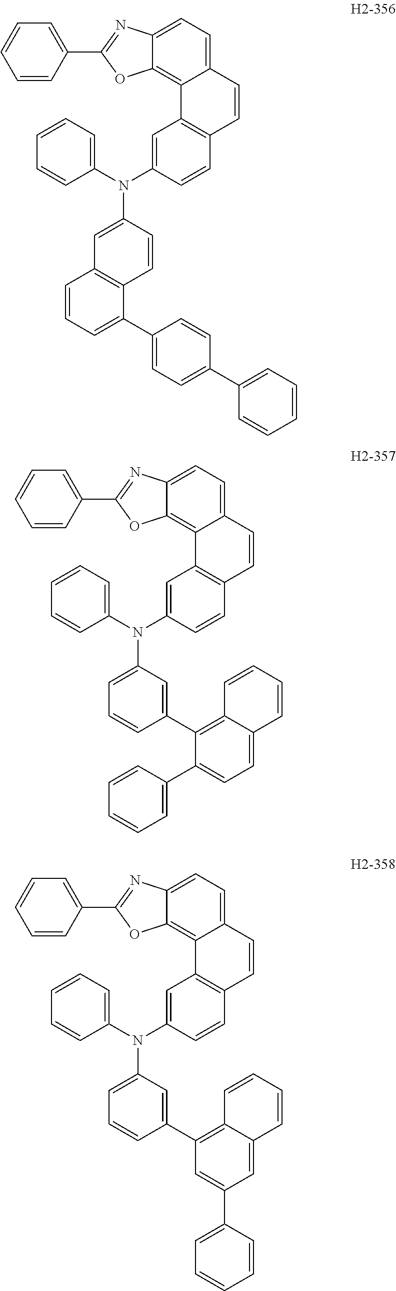 [SMILES: c1ccc(-c2ccc(-c3cccc4cc(N(c5ccccc5)c5ccc6ccc7ccc8nc(-c9ccccc9)oc8c7c6c5)ccc34)cc2)cc1 ; c1ccc(-c2nc3ccc4ccc5ccc(N(c6ccccc6)c6cccc(-c7c(-c8ccccc8)ccc8ccccc78)c6)cc5c4c3o2)cc1 ; c1ccc(-c2cc(-c3cccc(N(c4ccccc4)c4ccc5ccc6ccc7nc(-c8ccccc8)oc7c6c5c4)c3)c3ccccc3c2)cc1]

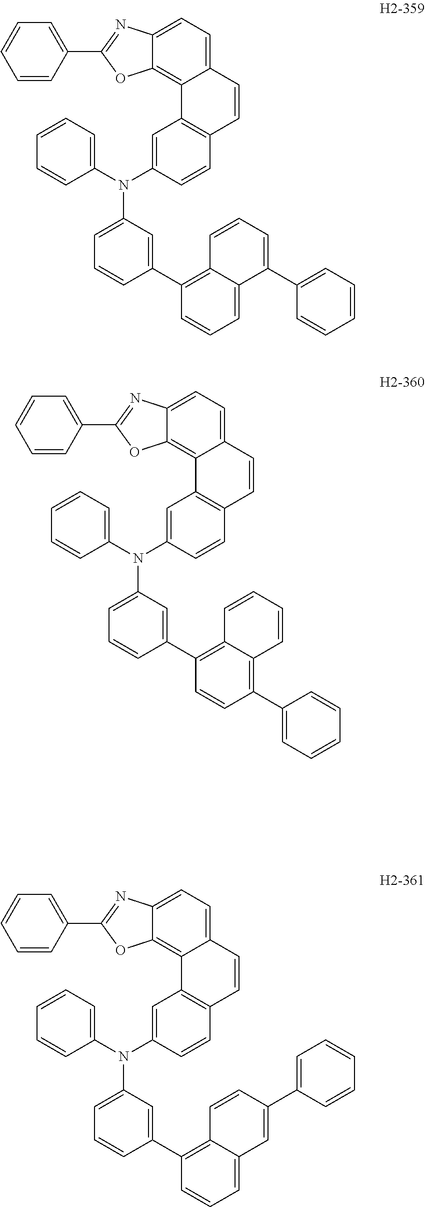 [SMILES: c1ccc(-c2nc3ccc4ccc5ccc(N(c6ccccc6)c6cccc(-c7cccc8c(-c9ccccc9)cccc78)c6)cc5c4c3o2)cc1 ; c1ccc(-c2nc3ccc4ccc5ccc(N(c6ccccc6)c6cccc(-c7ccc(-c8ccccc8)c8ccccc78)c6)cc5c4c3o2)cc1 ; c1ccc(-c2ccc3c(-c4cccc(N(c5ccccc5)c5ccc6ccc7ccc8nc(-c9ccccc9)oc8c7c6c5)c4)cccc3c2)cc1]

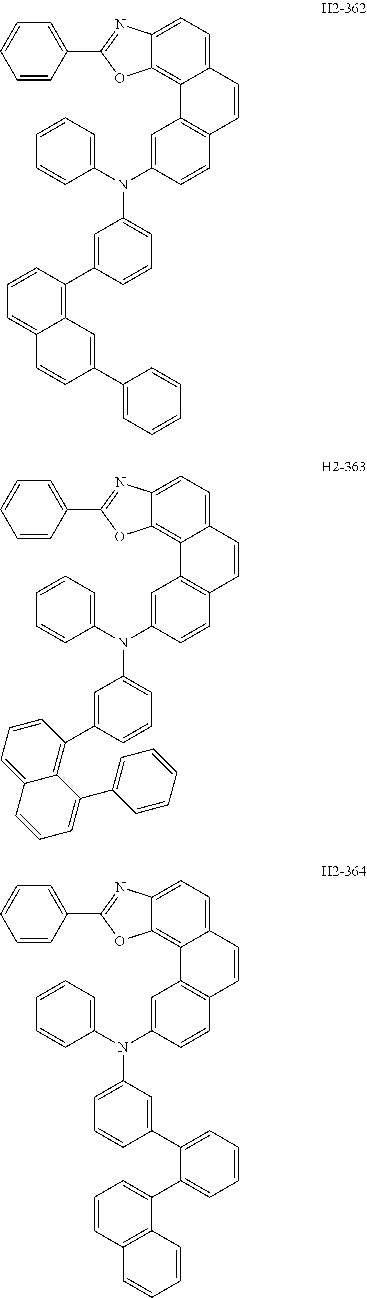 [SMILES: c1ccc(-c2ccc3cccc(-c4cccc(N(c5ccccc5)c5ccc6ccc7ccc8nc(-c9ccccc9)oc8c7c6c5)c4)c3c2)cc1 ; c1ccc(-c2nc3ccc4ccc5ccc(N(c6ccccc6)c6cccc(-c7cccc8cccc(-c9ccccc9)c78)c6)cc5c4c3o2)cc1 ; c1ccc(-c2nc3ccc4ccc5ccc(N(c6ccccc6)c6cccc(-c7ccccc7-c7cccc8ccccc78)c6)cc5c4c3o2)cc1]

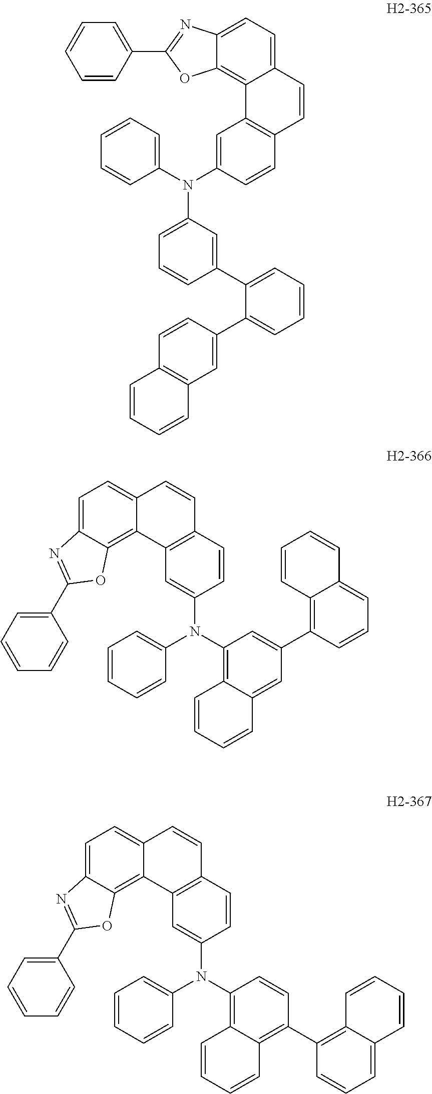 [SMILES: c1ccc(-c2nc3ccc4ccc5ccc(N(c6ccccc6)c6cccc(-c7ccccc7-c7ccc8ccccc8c7)c6)cc5c4c3o2)cc1 ; c1ccc(-c2nc3ccc4ccc5ccc(N(c6ccccc6)c6cc(-c7cccc8ccccc78)cc7ccccc67)cc5c4c3o2)cc1 ; c1ccc(-c2nc3ccc4ccc5ccc(N(c6ccccc6)c6ccc(-c7cccc8ccccc78)c7ccccc67)cc5c4c3o2)cc1]

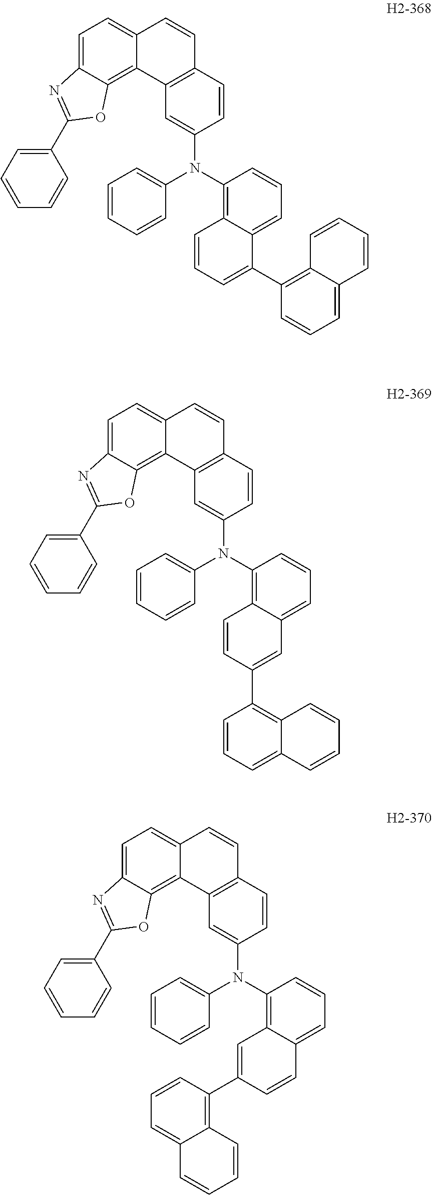 [SMILES: c1ccc(-c2nc3ccc4ccc5ccc(N(c6ccccc6)c6cccc7c(-c8cccc9ccccc89)cccc67)cc5c4c3o2)cc1 ; c1ccc(-c2nc3ccc4ccc5ccc(N(c6ccccc6)c6cccc7cc(-c8cccc9ccccc89)ccc67)cc5c4c3o2)cc1 ; c1ccc(-c2nc3ccc4ccc5ccc(N(c6ccccc6)c6cccc7ccc(-c8cccc9ccccc89)cc67)cc5c4c3o2)cc1]

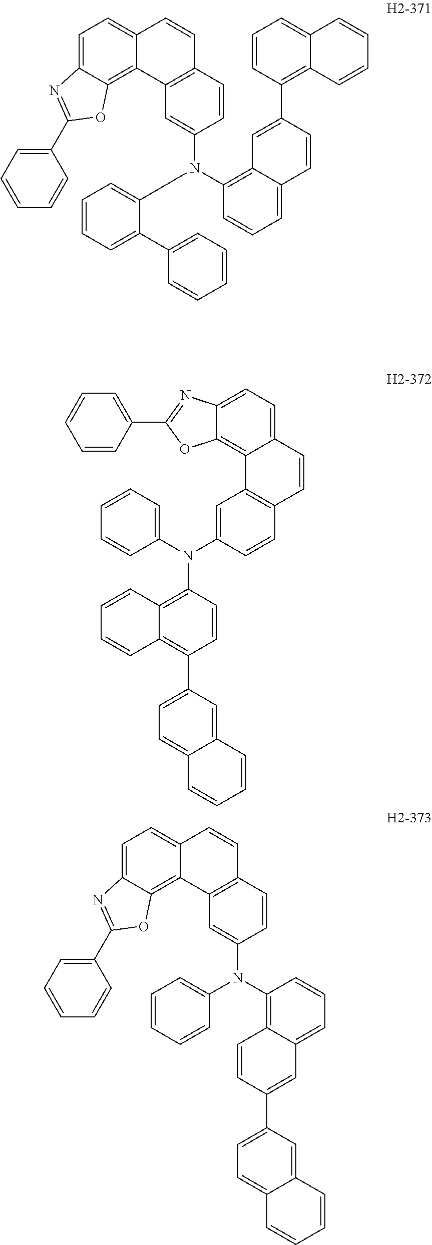

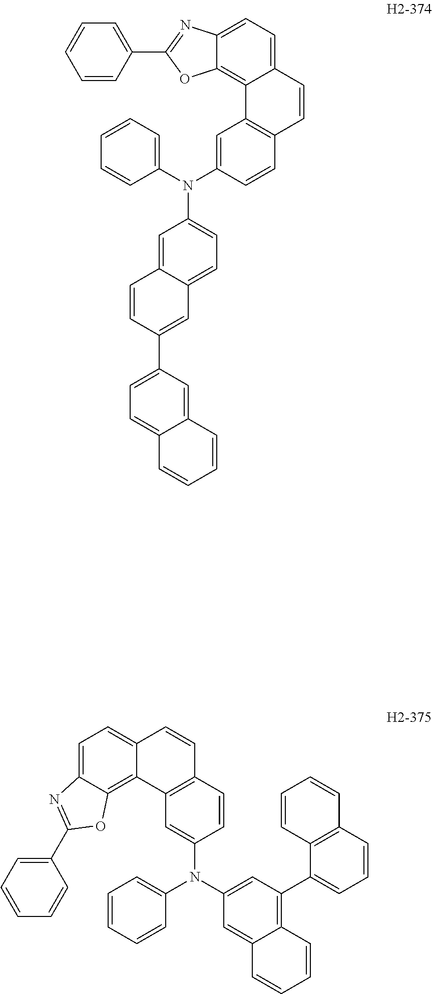 [SMILES: c1ccc(-c2nc3ccc4ccc5ccc(N(c6ccccc6)c6ccc7cc(-c8ccc9ccccc9c8)ccc7c6)cc5c4c3o2)cc1 ; c1ccc(-c2nc3ccc4ccc5ccc(N(c6ccccc6)c6cc(-c7cccc8ccccc78)c7ccccc7c6)cc5c4c3o2)cc1]

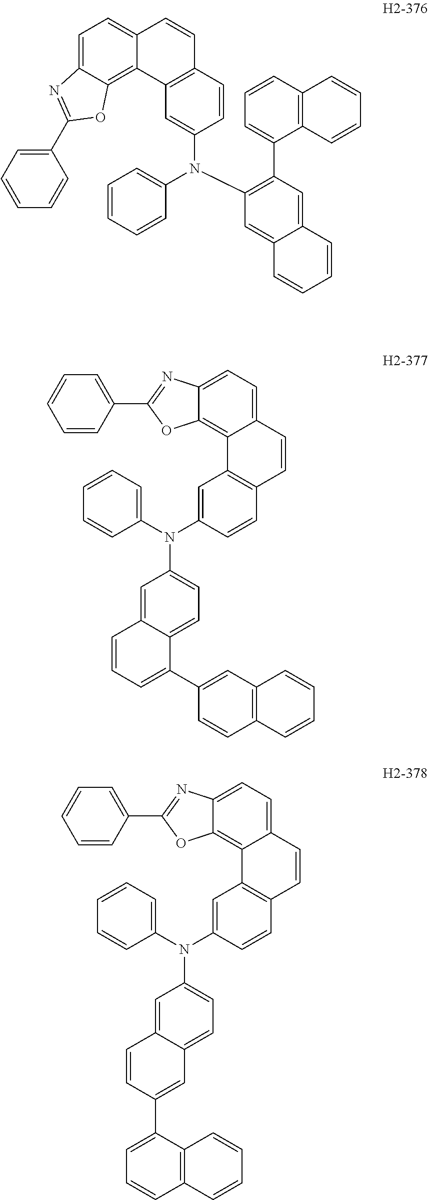 [SMILES: c1ccc(-c2nc3ccc4ccc5ccc(N(c6ccccc6)c6cc7ccccc7cc6-c6cccc7ccccc67)cc5c4c3o2)cc1 ; c1ccc(-c2nc3ccc4ccc5ccc(N(c6ccccc6)c6ccc7c(-c8ccc9ccccc9c8)cccc7c6)cc5c4c3o2)cc1 ; c1ccc(-c2nc3ccc4ccc5ccc(N(c6ccccc6)c6ccc7cc(-c8cccc9ccccc89)ccc7c6)cc5c4c3o2)cc1]

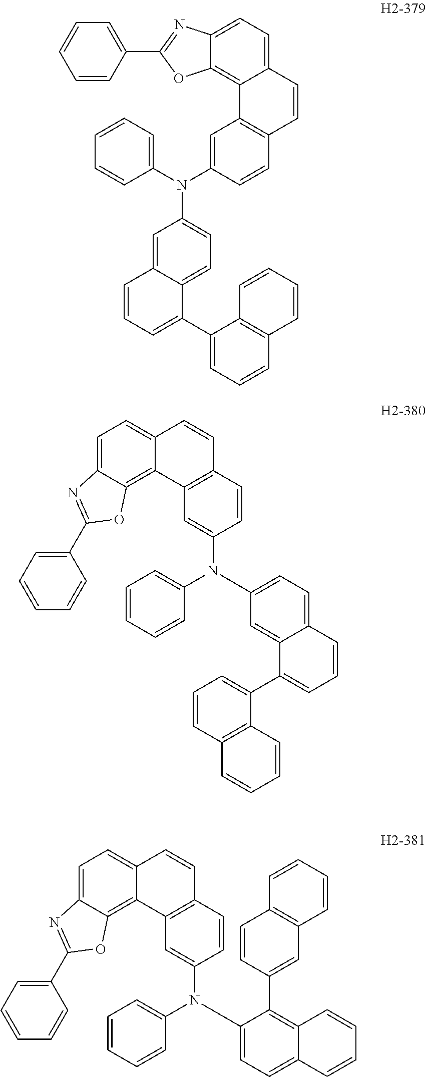 [SMILES: c1ccc(-c2nc3ccc4ccc5ccc(N(c6ccccc6)c6ccc7c(-c8cccc9ccccc89)cccc7c6)cc5c4c3o2)cc1 ; c1ccc(-c2nc3ccc4ccc5ccc(N(c6ccccc6)c6ccc7cccc(-c8cccc9ccccc89)c7c6)cc5c4c3o2)cc1 ; c1ccc(-c2nc3ccc4ccc5ccc(N(c6ccccc6)c6ccc7ccccc7c6-c6ccc7ccccc7c6)cc5c4c3o2)cc1]

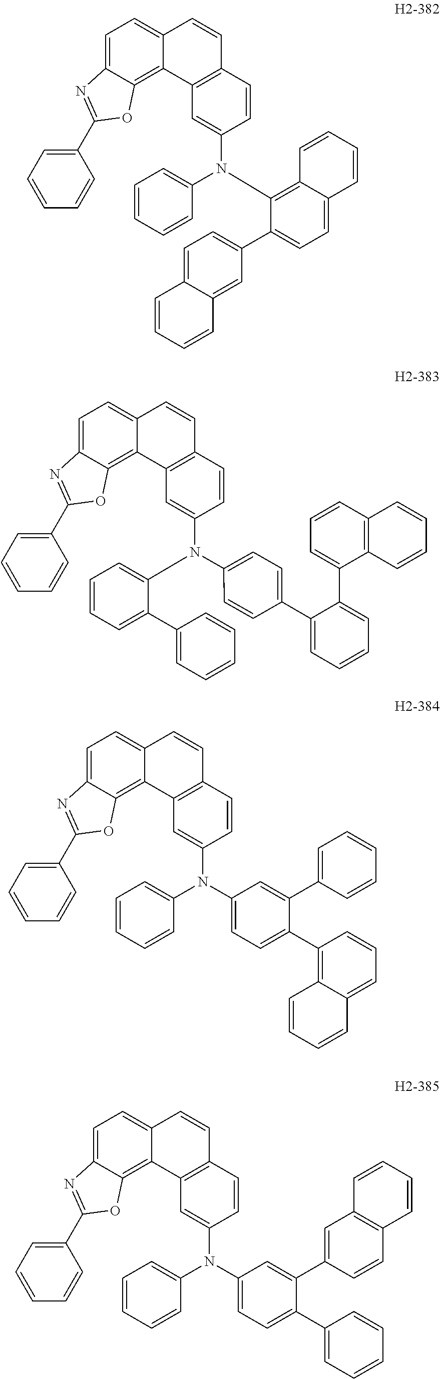 [SMILES: c1ccc(-c2nc3ccc4ccc5ccc(N(c6ccccc6)c6c(-c7ccc8ccccc8c7)ccc7ccccc67)cc5c4c3o2)cc1 ; c1ccc(-c2nc3ccc4ccc5ccc(N(c6ccc(-c7ccccc7-c7cccc8ccccc78)cc6)c6ccccc6-c6ccccc6)cc5c4c3o2)cc1 ; c1ccc(-c2nc3ccc4ccc5ccc(N(c6ccccc6)c6ccc(-c7cccc8ccccc78)c(-c7ccccc7)c6)cc5c4c3o2)cc1 ; c1ccc(-c2nc3ccc4ccc5ccc(N(c6ccccc6)c6ccc(-c7ccccc7)c(-c7ccc8ccccc8c7)c6)cc5c4c3o2)cc1]

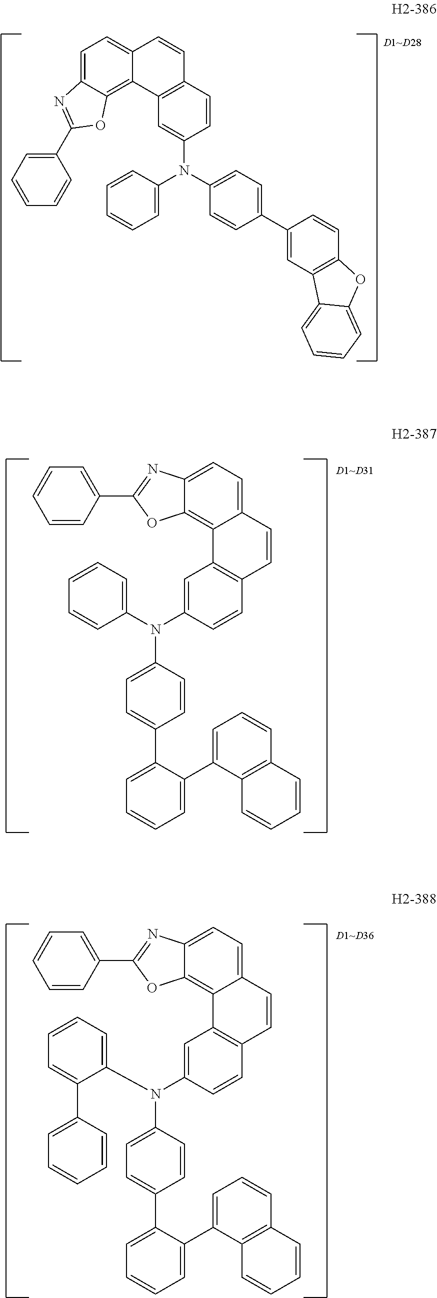

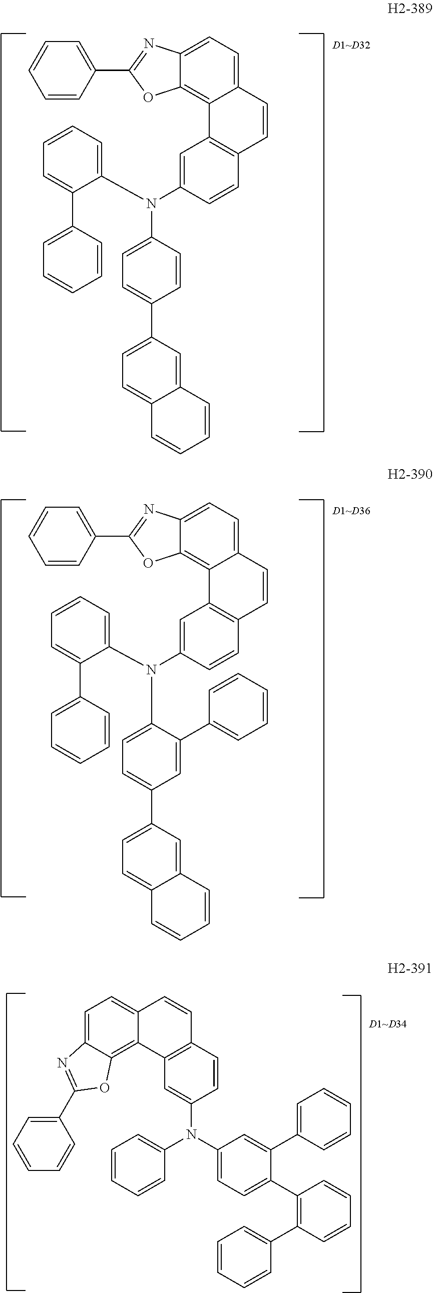

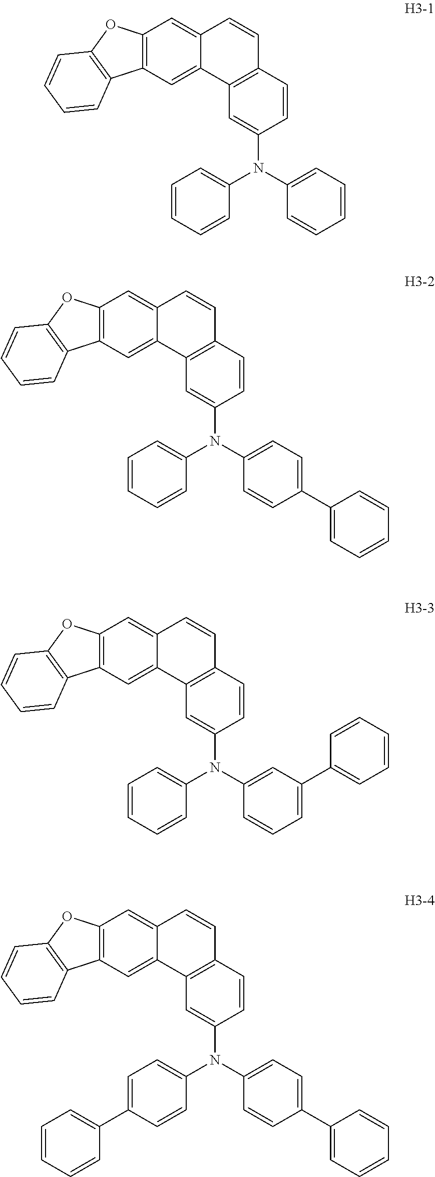 [SMILES: c1ccc(N(c2ccccc2)c2ccc3ccc4cc5oc6ccccc6c5cc4c3c2)cc1 ; c1ccc(-c2ccc(N(c3ccccc3)c3ccc4ccc5cc6oc7ccccc7c6cc5c4c3)cc2)cc1 ; c1ccc(-c2cccc(N(c3ccccc3)c3ccc4ccc5cc6oc7ccccc7c6cc5c4c3)c2)cc1 ; c1ccc(-c2ccc(N(c3ccc(-c4ccccc4)cc3)c3ccc4ccc5cc6oc7ccccc7c6cc5c4c3)cc2)cc1]

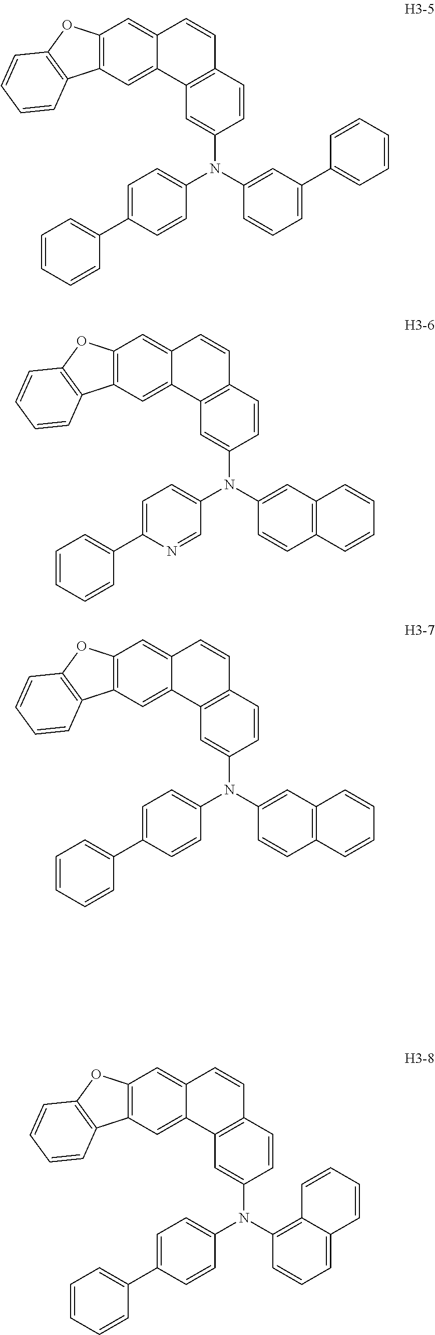

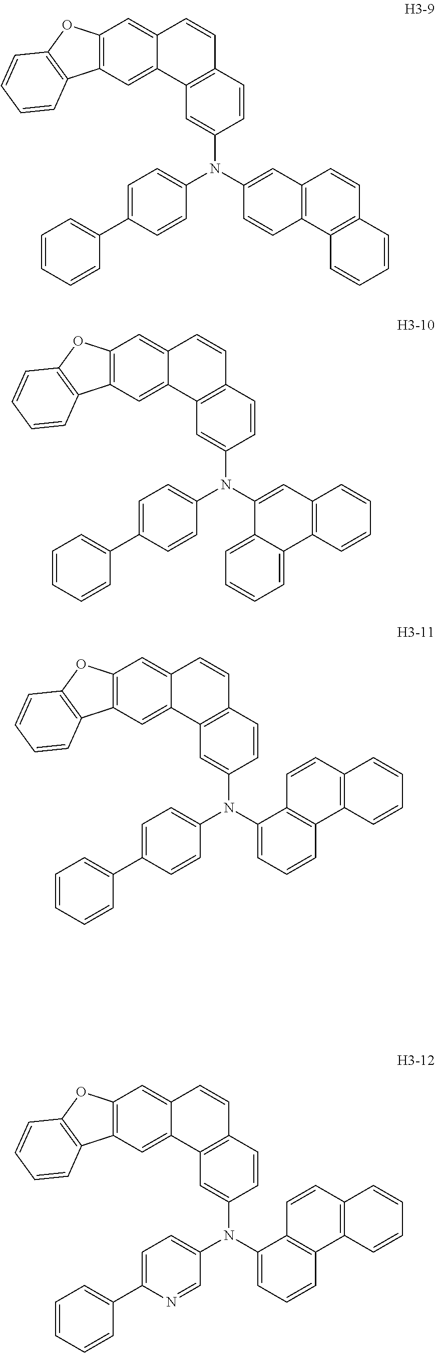 [SMILES: c1ccc(-c2ccc(N(c3ccc4c(ccc5ccccc54)c3)c3ccc4ccc5cc6oc7ccccc7c6cc5c4c3)cc2)cc1 ; c1ccc(-c2ccc(N(c3ccc4ccc5cc6oc7ccccc7c6cc5c4c3)c3cc4ccccc4c4ccccc34)cc2)cc1 ; c1ccc(-c2ccc(N(c3ccc4ccc5cc6oc7ccccc7c6cc5c4c3)c3cccc4c3ccc3ccccc34)cc2)cc1 ; c1ccc(-c2ccc(N(c3ccc4ccc5cc6oc7ccccc7c6cc5c4c3)c3cccc4c3ccc3ccccc34)cn2)cc1]

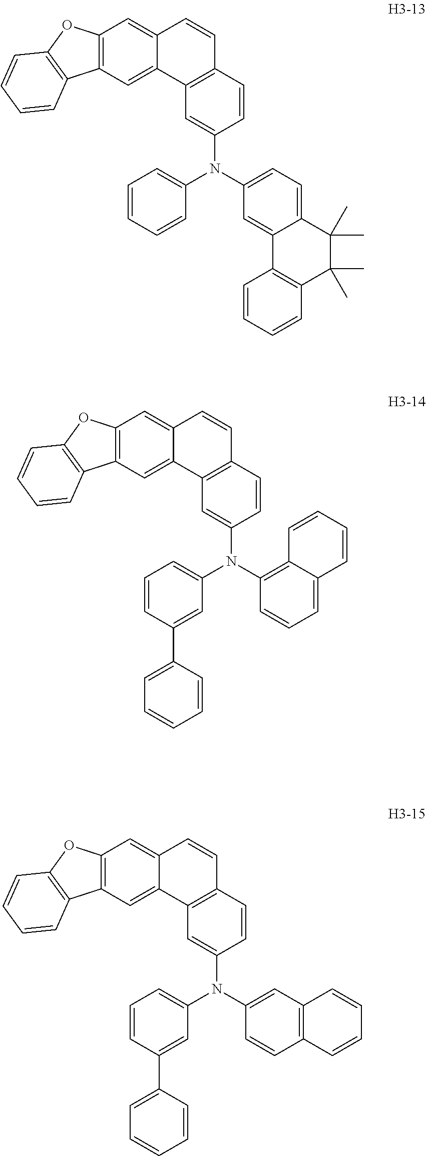 [SMILES: CC1(C)c2ccccc2-c2cc(N(c3ccccc3)c3ccc4ccc5cc6oc7ccccc7c6cc5c4c3)ccc2C1(C)C ; c1ccc(-c2cccc(N(c3ccc4ccc5cc6oc7ccccc7c6cc5c4c3)c3cccc4ccccc34)c2)cc1 ; c1ccc(-c2cccc(N(c3ccc4ccccc4c3)c3ccc4ccc5cc6oc7ccccc7c6cc5c4c3)c2)cc1]

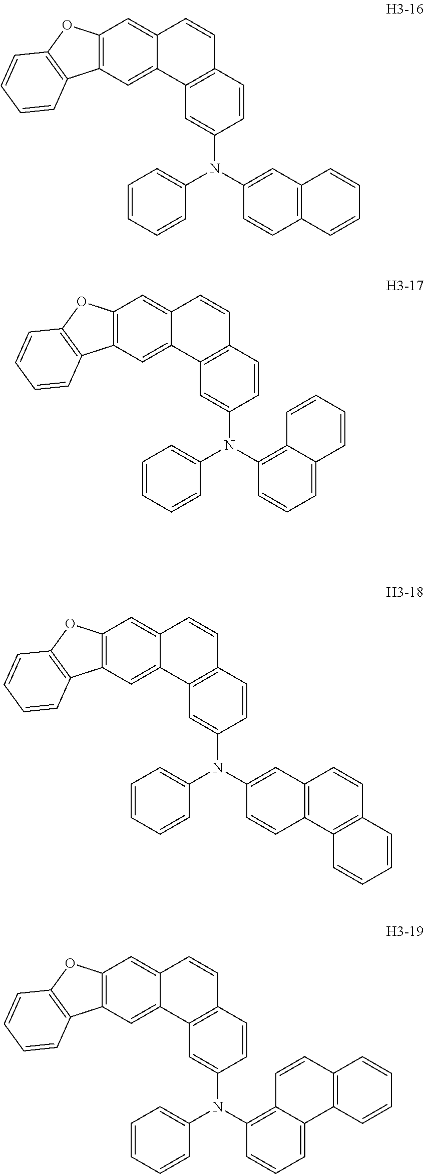 [SMILES: c1ccc(N(c2ccc3ccccc3c2)c2ccc3ccc4cc5oc6ccccc6c5cc4c3c2)cc1 ; c1ccc(N(c2ccc3ccc4cc5oc6ccccc6c5cc4c3c2)c2cccc3ccccc23)cc1 ; c1ccc(N(c2ccc3c(ccc4ccccc43)c2)c2ccc3ccc4cc5oc6ccccc6c5cc4c3c2)cc1 ; c1ccc(N(c2ccc3ccc4cc5oc6ccccc6c5cc4c3c2)c2cccc3c2ccc2ccccc23)cc1]

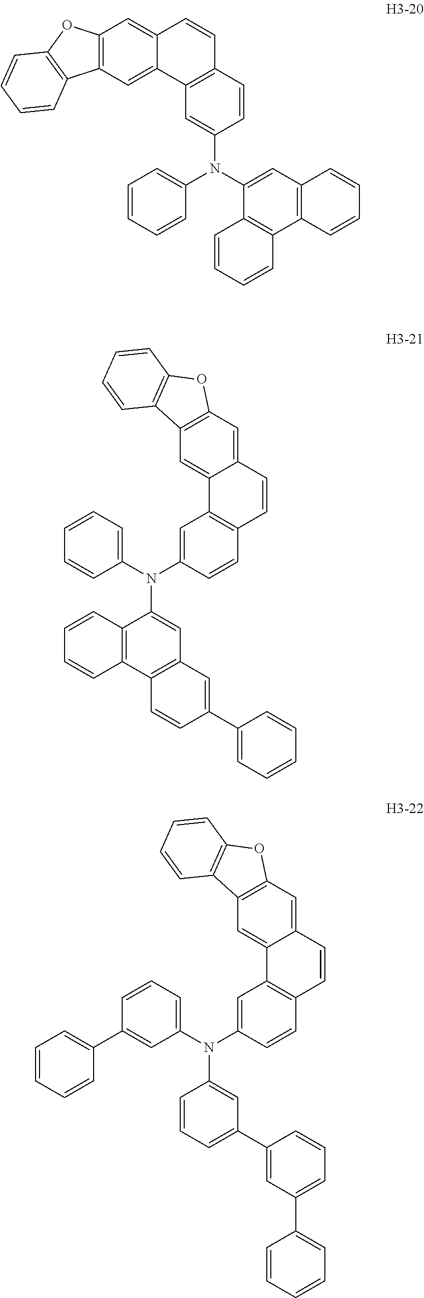 [SMILES: c1ccc(N(c2ccc3ccc4cc5oc6ccccc6c5cc4c3c2)c2cc3ccccc3c3ccccc23)cc1 ; c1ccc(-c2ccc3c(c2)cc(N(c2ccccc2)c2ccc4ccc5cc6oc7ccccc7c6cc5c4c2)c2ccccc23)cc1 ; c1ccc(-c2cccc(-c3cccc(N(c4cccc(-c5ccccc5)c4)c4ccc5ccc6cc7oc8ccccc8c7cc6c5c4)c3)c2)cc1]

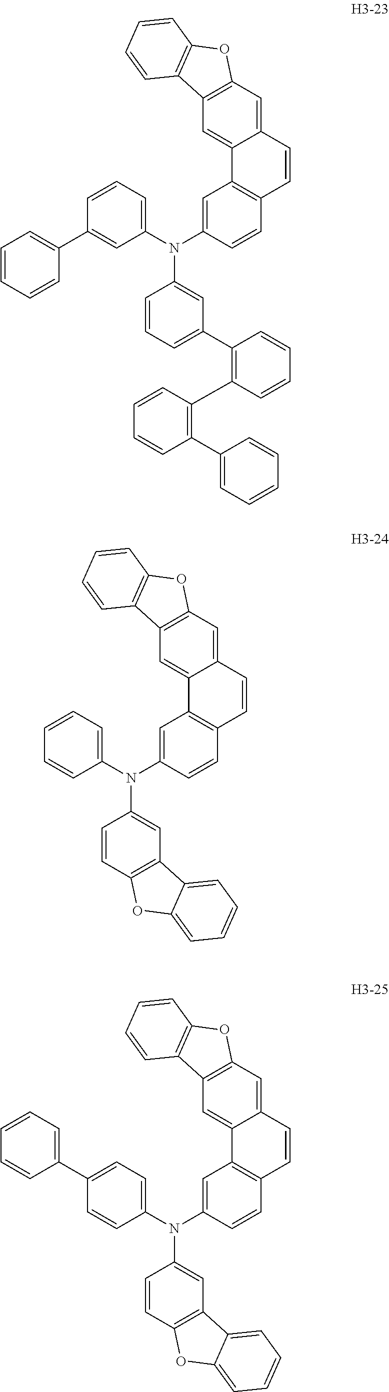 [SMILES: c1ccc(-c2cccc(N(c3cccc(-c4ccccc4-c4ccccc4-c4ccccc4)c3)c3ccc4ccc5cc6oc7ccccc7c6cc5c4c3)c2)cc1 ; c1ccc(N(c2ccc3ccc4cc5oc6ccccc6c5cc4c3c2)c2ccc3oc4ccccc4c3c2)cc1 ; c1ccc(-c2ccc(N(c3ccc4ccc5cc6oc7ccccc7c6cc5c4c3)c3ccc4oc5ccccc5c4c3)cc2)cc1]

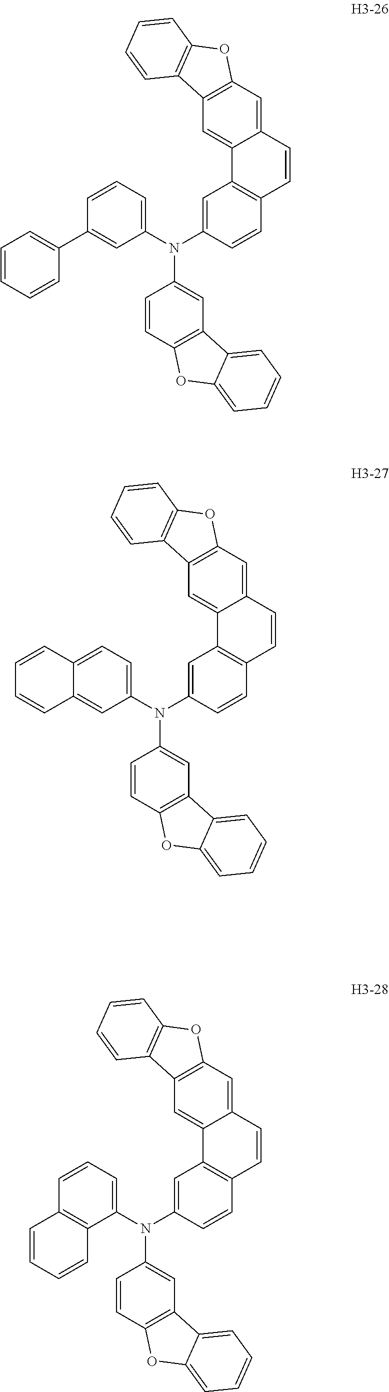 [SMILES: c1ccc(-c2cccc(N(c3ccc4ccc5cc6oc7ccccc7c6cc5c4c3)c3ccc4oc5ccccc5c4c3)c2)cc1 ; c1ccc2cc(N(c3ccc4ccc5cc6oc7ccccc7c6cc5c4c3)c3ccc4oc5ccccc5c4c3)ccc2c1 ; c1ccc2c(N(c3ccc4ccc5cc6oc7ccccc7c6cc5c4c3)c3ccc4oc5ccccc5c4c3)cccc2c1]

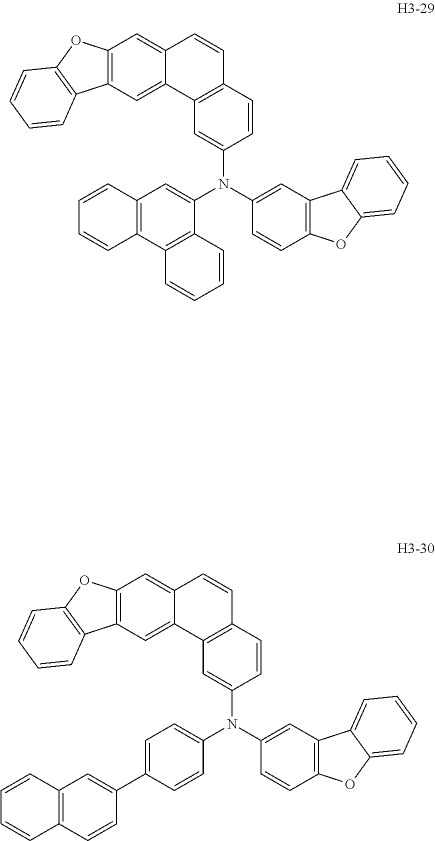 [SMILES: c1ccc2c(c1)cc(N(c1ccc3ccc4cc5oc6ccccc6c5cc4c3c1)c1ccc3oc4ccccc4c3c1)c1ccccc12 ; c1ccc2cc(-c3ccc(N(c4ccc5ccc6cc7oc8ccccc8c7cc6c5c4)c4ccc5oc6ccccc6c5c4)cc3)ccc2c1]

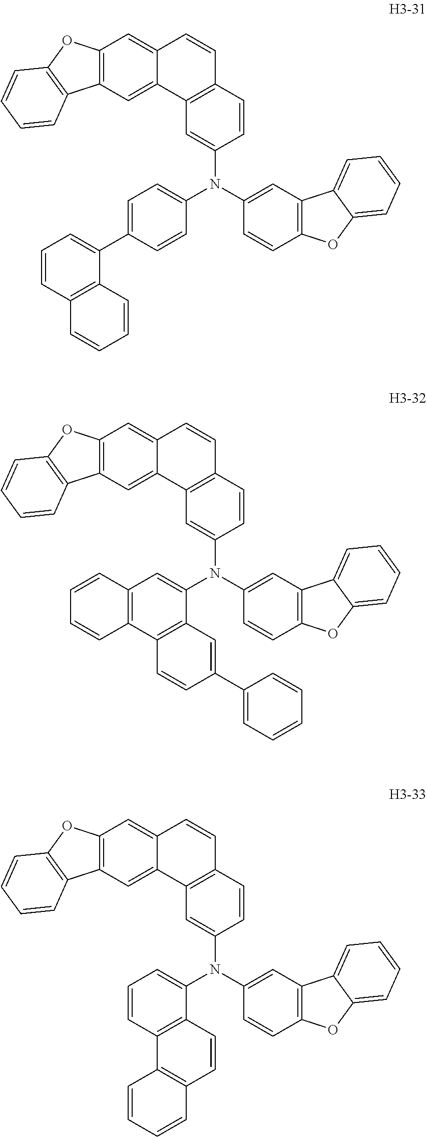 [SMILES: c1ccc2c(-c3ccc(N(c4ccc5ccc6cc7oc8ccccc8c7cc6c5c4)c4ccc5oc6ccccc6c5c4)cc3)cccc2c1 ; c1ccc(-c2ccc3c(c2)c(N(c2ccc4ccc5cc6oc7ccccc7c6cc5c4c2)c2ccc4oc5ccccc5c4c2)cc2ccccc23)cc1 ; c1ccc2c(c1)ccc1c(N(c3ccc4ccc5cc6oc7ccccc7c6cc5c4c3)c3ccc4oc5ccccc5c4c3)cccc12]

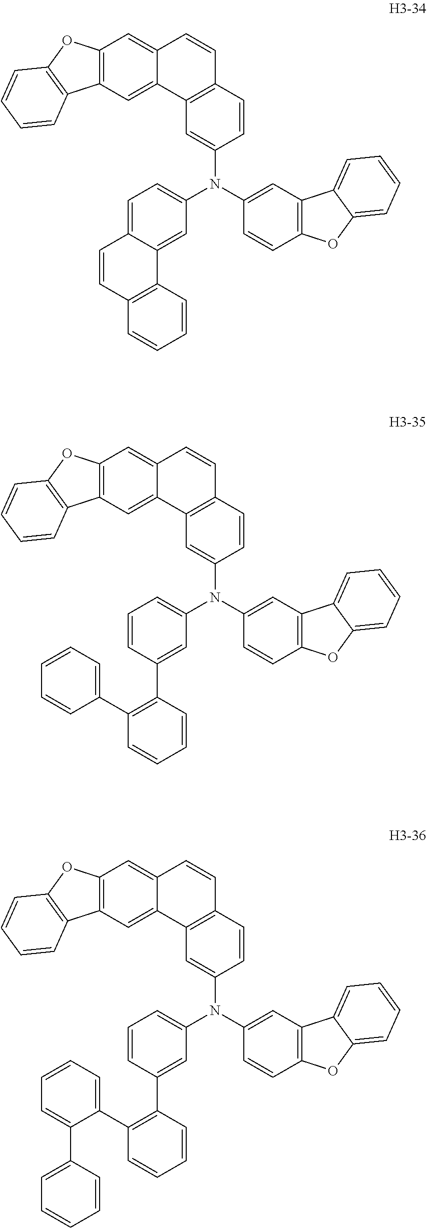 [SMILES: c1ccc2c(c1)ccc1ccc(N(c3ccc4ccc5cc6oc7ccccc7c6cc5c4c3)c3ccc4oc5ccccc5c4c3)cc12 ; c1ccc(-c2ccccc2-c2cccc(N(c3ccc4ccc5cc6oc7ccccc7c6cc5c4c3)c3ccc4oc5ccccc5c4c3)c2)cc1 ; c1ccc(-c2ccccc2-c2ccccc2-c2cccc(N(c3ccc4ccc5cc6oc7ccccc7c6cc5c4c3)c3ccc4oc5ccccc5c4c3)c2)cc1]

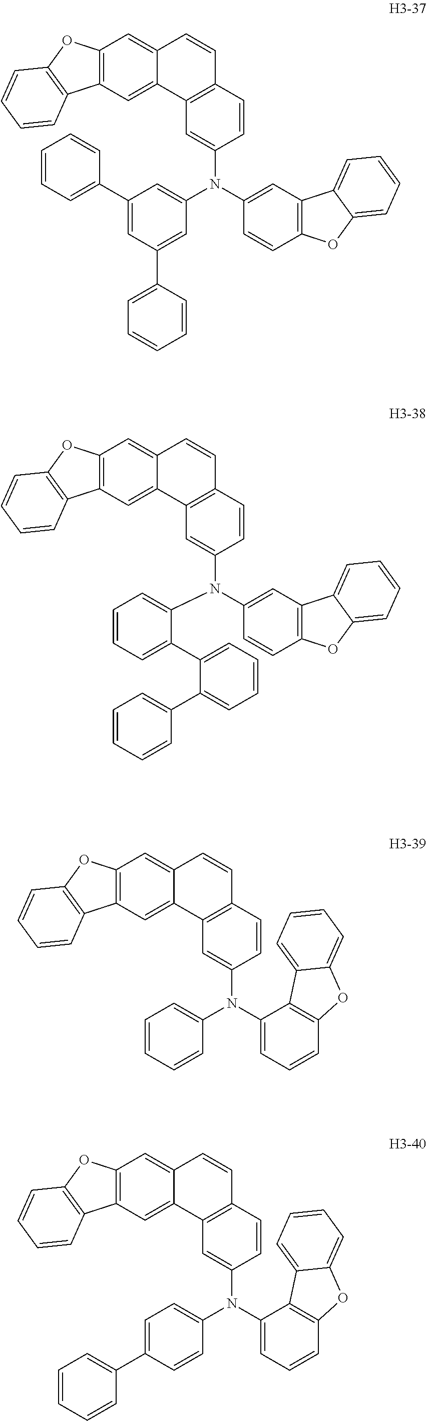 [SMILES: c1ccc(-c2cc(-c3ccccc3)cc(N(c3ccc4ccc5cc6oc7ccccc7c6cc5c4c3)c3ccc4oc5ccccc5c4c3)c2)cc1 ; c1ccc(-c2ccccc2-c2ccccc2N(c2ccc3ccc4cc5oc6ccccc6c5cc4c3c2)c2ccc3oc4ccccc4c3c2)cc1 ; c1ccc(N(c2ccc3ccc4cc5oc6ccccc6c5cc4c3c2)c2cccc3oc4ccccc4c23)cc1 ; c1ccc(-c2ccc(N(c3ccc4ccc5cc6oc7ccccc7c6cc5c4c3)c3cccc4oc5ccccc5c34)cc2)cc1]

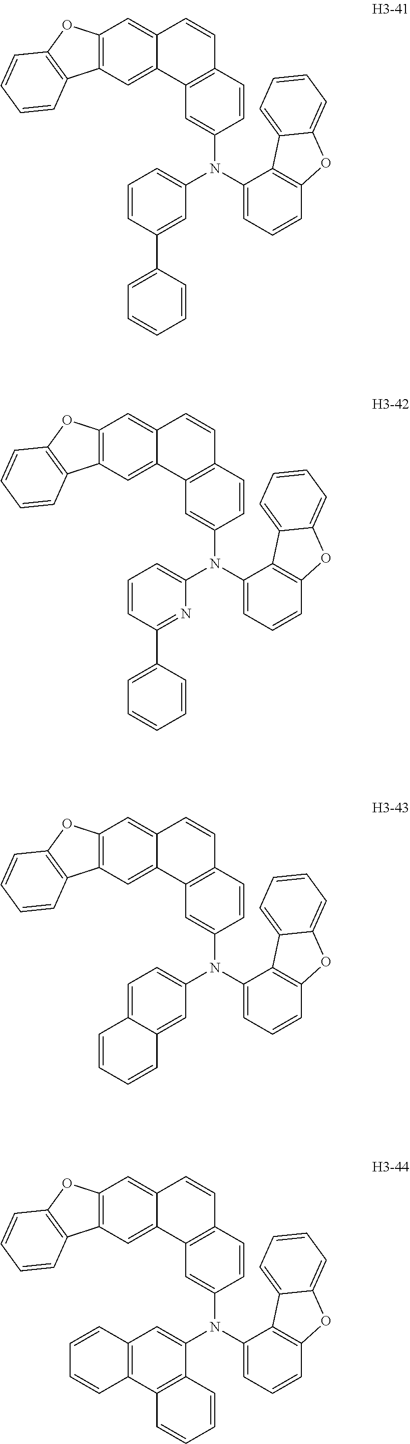 [SMILES: c1ccc(-c2cccc(N(c3ccc4ccc5cc6oc7ccccc7c6cc5c4c3)c3cccc4oc5ccccc5c34)c2)cc1 ; c1ccc(-c2cccc(N(c3ccc4ccc5cc6oc7ccccc7c6cc5c4c3)c3cccc4oc5ccccc5c34)n2)cc1 ; c1ccc2cc(N(c3ccc4ccc5cc6oc7ccccc7c6cc5c4c3)c3cccc4oc5ccccc5c34)ccc2c1 ; c1ccc2c(c1)cc(N(c1ccc3ccc4cc5oc6ccccc6c5cc4c3c1)c1cccc3oc4ccccc4c13)c1ccccc12]

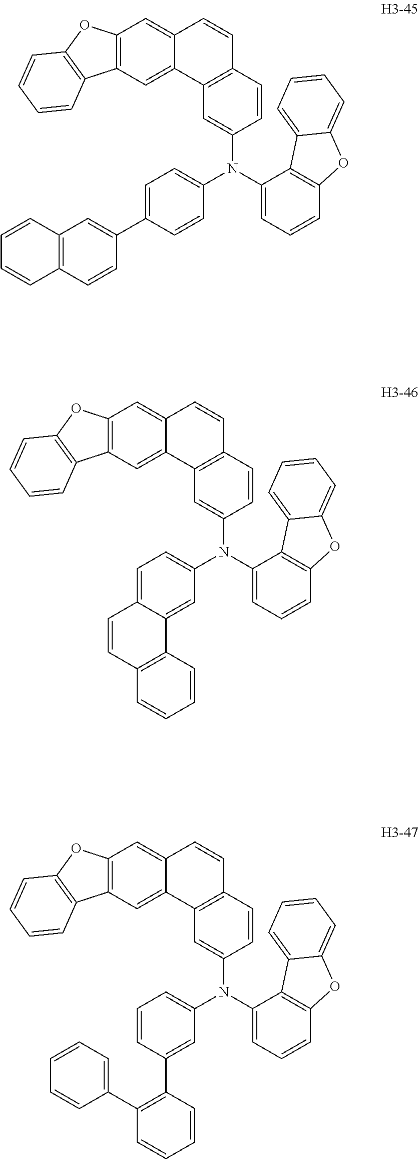 [SMILES: c1ccc2cc(-c3ccc(N(c4ccc5ccc6cc7oc8ccccc8c7cc6c5c4)c4cccc5oc6ccccc6c45)cc3)ccc2c1 ; c1ccc2c(c1)ccc1ccc(N(c3ccc4ccc5cc6oc7ccccc7c6cc5c4c3)c3cccc4oc5ccccc5c34)cc12 ; c1ccc(-c2ccccc2-c2cccc(N(c3ccc4ccc5cc6oc7ccccc7c6cc5c4c3)c3cccc4oc5ccccc5c34)c2)cc1]

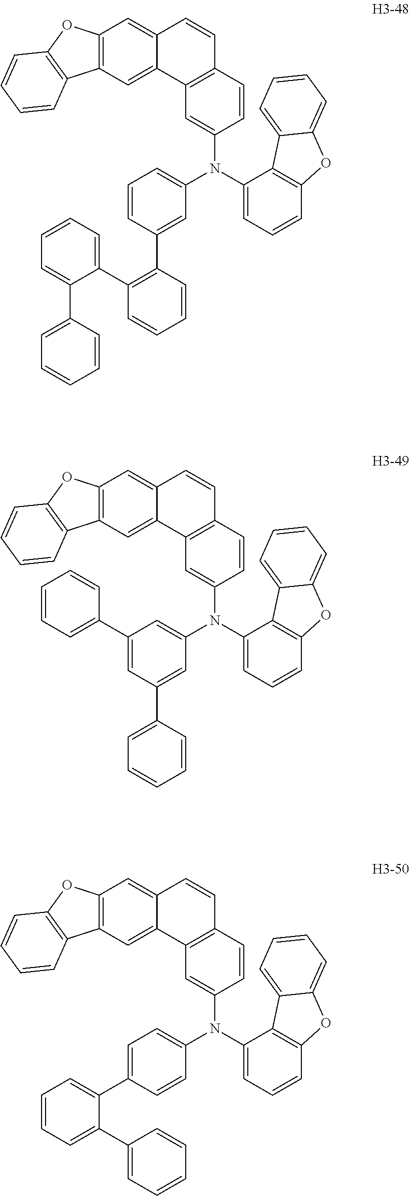 [SMILES: c1ccc(-c2ccccc2-c2ccccc2-c2cccc(N(c3ccc4ccc5cc6oc7ccccc7c6cc5c4c3)c3cccc4oc5ccccc5c34)c2)cc1 ; c1ccc(-c2cc(-c3ccccc3)cc(N(c3ccc4ccc5cc6oc7ccccc7c6cc5c4c3)c3cccc4oc5ccccc5c34)c2)cc1 ; c1ccc(-c2ccccc2-c2ccc(N(c3ccc4ccc5cc6oc7ccccc7c6cc5c4c3)c3cccc4oc5ccccc5c34)cc2)cc1]

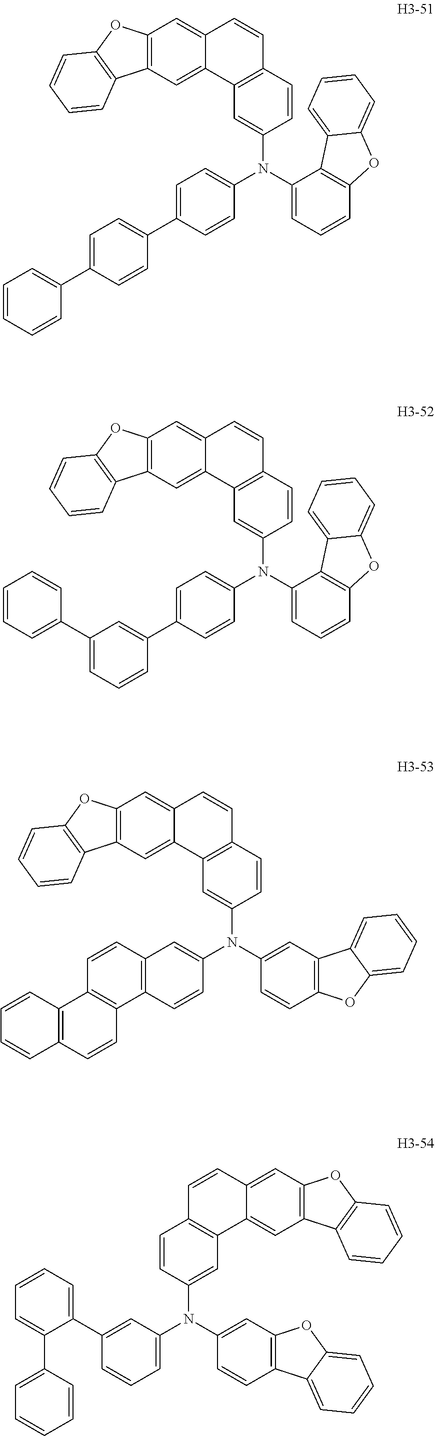 [SMILES: c1ccc(-c2ccc(-c3ccc(N(c4ccc5ccc6cc7oc8ccccc8c7cc6c5c4)c4cccc5oc6ccccc6c45)cc3)cc2)cc1 ; c1ccc(-c2cccc(-c3ccc(N(c4ccc5ccc6cc7oc8ccccc8c7cc6c5c4)c4cccc5oc6ccccc6c45)cc3)c2)cc1 ; c1ccc2c(c1)ccc1c3ccc(N(c4ccc5ccc6cc7oc8ccccc8c7cc6c5c4)c4ccc5oc6ccccc6c5c4)cc3ccc21 ; c1ccc(-c2ccccc2-c2cccc(N(c3ccc4c(c3)oc3ccccc34)c3ccc4ccc5cc6oc7ccccc7c6cc5c4c3)c2)cc1]

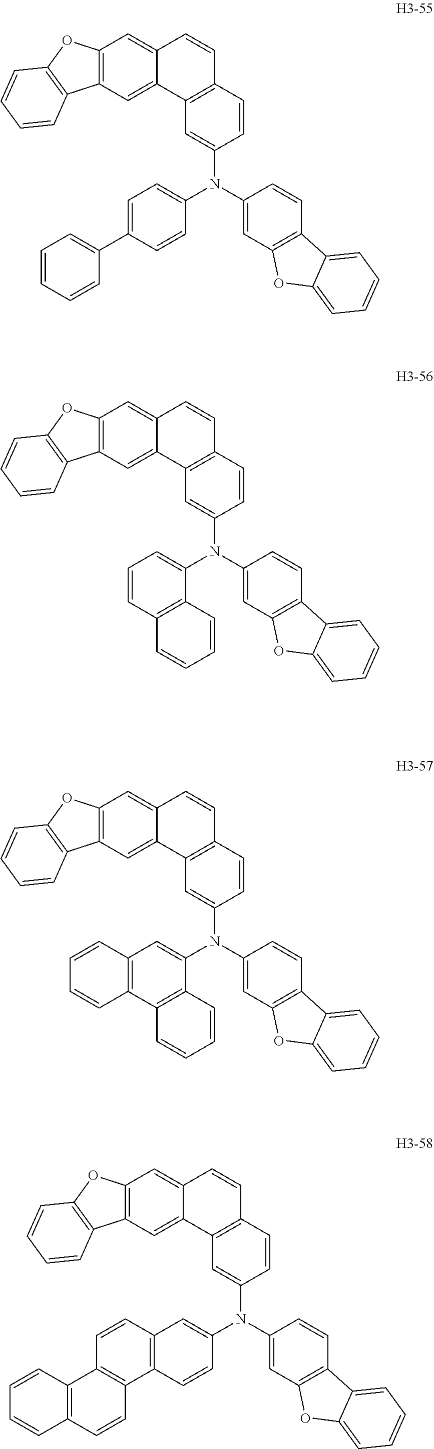 [SMILES: c1ccc(-c2ccc(N(c3ccc4c(c3)oc3ccccc34)c3ccc4ccc5cc6oc7ccccc7c6cc5c4c3)cc2)cc1 ; c1ccc2c(N(c3ccc4c(c3)oc3ccccc34)c3ccc4ccc5cc6oc7ccccc7c6cc5c4c3)cccc2c1 ; c1ccc2c(c1)cc(N(c1ccc3c(c1)oc1ccccc13)c1ccc3ccc4cc5oc6ccccc6c5cc4c3c1)c1ccccc12 ; c1ccc2c(c1)ccc1c3ccc(N(c4ccc5c(c4)oc4ccccc45)c4ccc5ccc6cc7oc8ccccc8c7cc6c5c4)cc3ccc21]

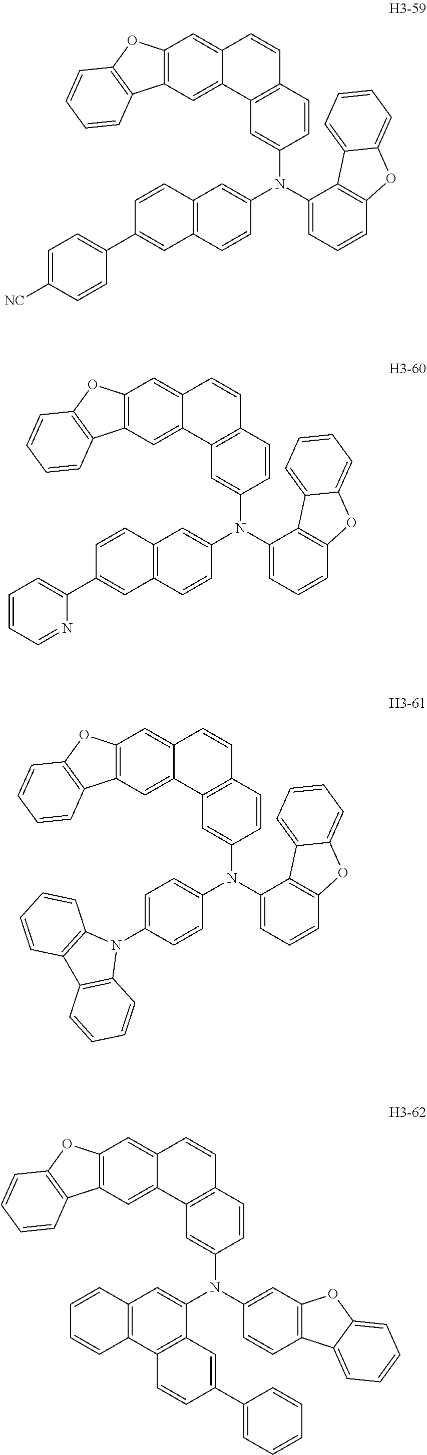 [SMILES: N#Cc1ccc(-c2ccc3cc(N(c4ccc5ccc6cc7oc8ccccc8c7cc6c5c4)c4cccc5oc6ccccc6c45)ccc3c2)cc1 ; c1ccc(-c2ccc3cc(N(c4ccc5ccc6cc7oc8ccccc8c7cc6c5c4)c4cccc5oc6ccccc6c45)ccc3c2)nc1 ; c1ccc2c(c1)oc1cc3ccc4ccc(N(c5ccc(-n6c7ccccc7c7ccccc76)cc5)c5cccc6oc7ccccc7c56)cc4c3cc12 ; c1ccc(-c2ccc3c(c2)c(N(c2ccc4c(c2)oc2ccccc24)c2ccc4ccc5cc6oc7ccccc7c6cc5c4c2)cc2ccccc23)cc1]

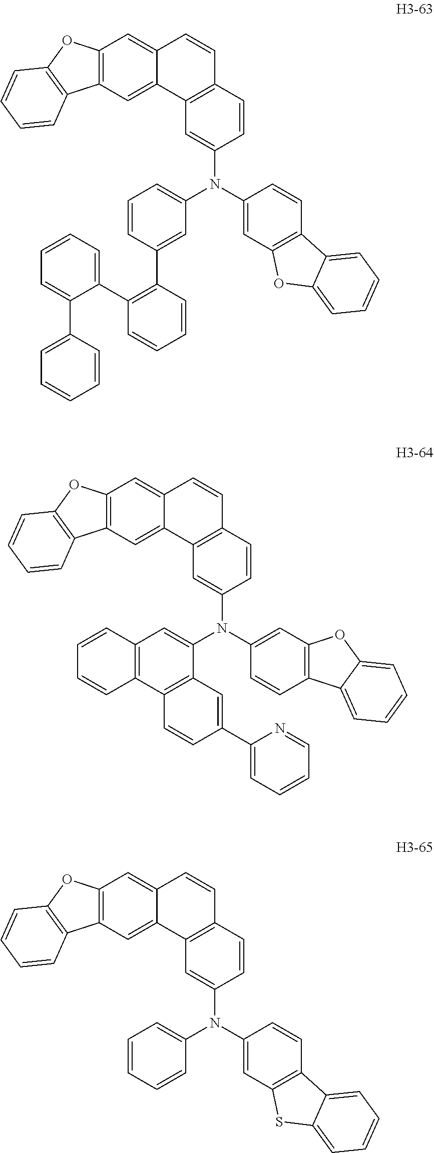 [SMILES: c1ccc(-c2ccccc2-c2ccccc2-c2cccc(N(c3ccc4c(c3)oc3ccccc34)c3ccc4ccc5cc6oc7ccccc7c6cc5c4c3)c2)cc1 ; c1ccc(-c2ccc3c(c2)c(N(c2ccc4c(c2)oc2ccccc24)c2ccc4ccc5cc6oc7ccccc7c6cc5c4c2)cc2ccccc23)nc1 ; c1ccc(N(c2ccc3c(c2)sc2ccccc23)c2ccc3ccc4cc5oc6ccccc6c5cc4c3c2)cc1]

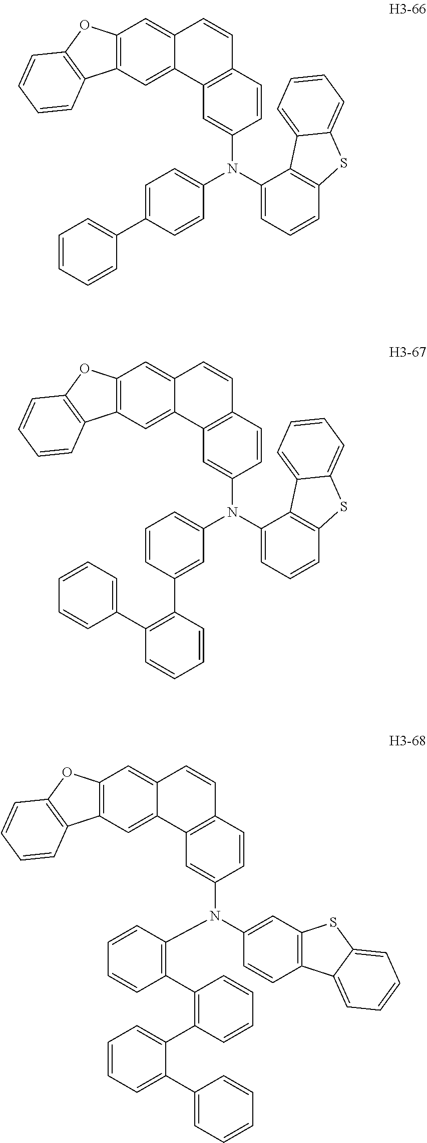 [SMILES: c1ccc(-c2ccc(N(c3ccc4ccc5cc6oc7ccccc7c6cc5c4c3)c3cccc4sc5ccccc5c34)cc2)cc1 ; c1ccc(-c2ccccc2-c2cccc(N(c3ccc4ccc5cc6oc7ccccc7c6cc5c4c3)c3cccc4sc5ccccc5c34)c2)cc1 ; c1ccc(-c2ccccc2-c2ccccc2-c2ccccc2N(c2ccc3c(c2)sc2ccccc23)c2ccc3ccc4cc5oc6ccccc6c5cc4c3c2)cc1]

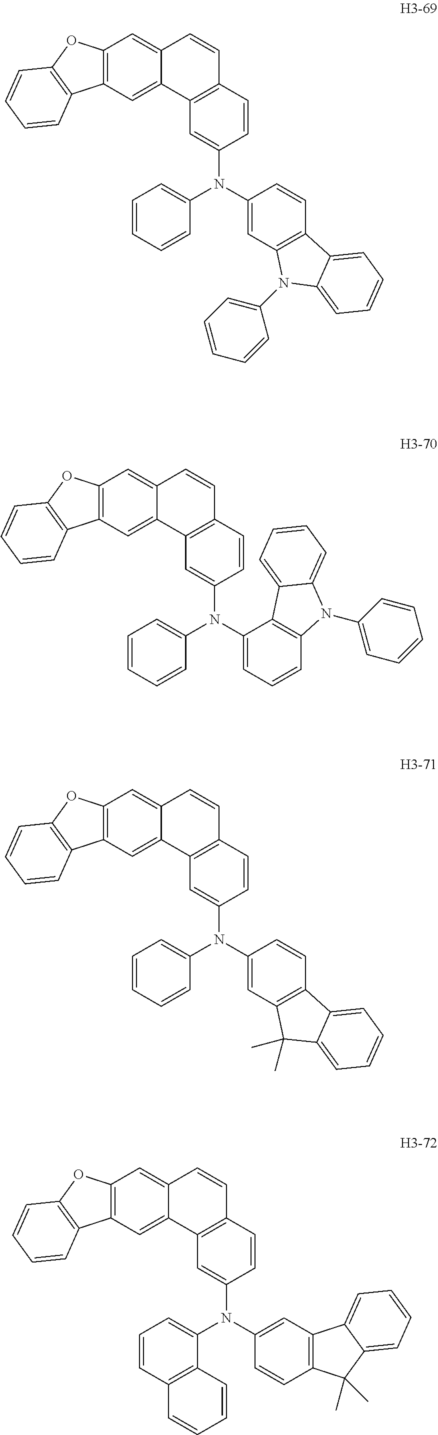 [SMILES: c1ccc(N(c2ccc3ccc4cc5oc6ccccc6c5cc4c3c2)c2ccc3c4ccccc4n(-c4ccccc4)c3c2)cc1 ; c1ccc(N(c2ccc3ccc4cc5oc6ccccc6c5cc4c3c2)c2cccc3c2c2ccccc2n3-c2ccccc2)cc1 ; CC1(C)c2ccccc2-c2ccc(N(c3ccccc3)c3ccc4ccc5cc6oc7ccccc7c6cc5c4c3)cc21 ; CC1(C)c2ccccc2-c2cc(N(c3ccc4ccc5cc6oc7ccccc7c6cc5c4c3)c3cccc4ccccc34)ccc21]

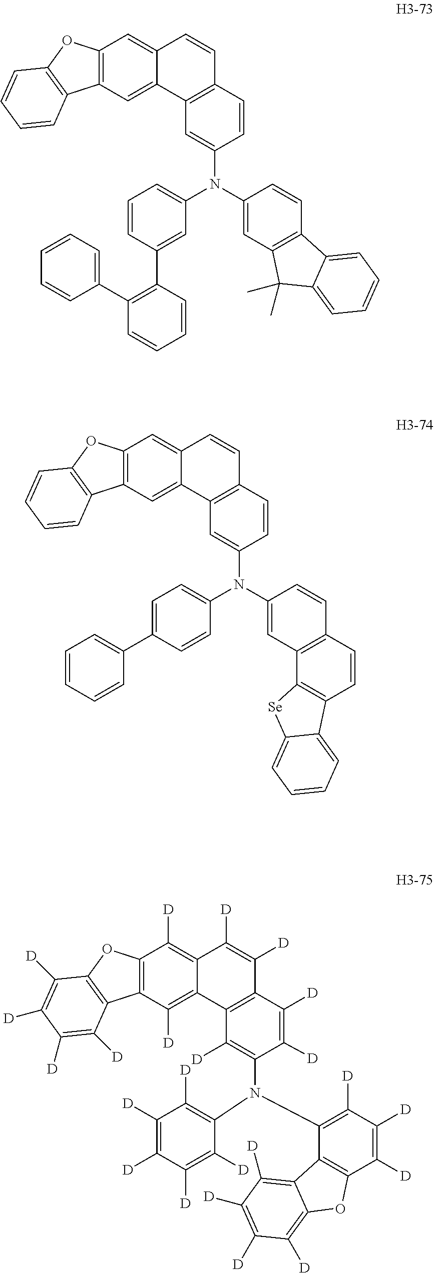 [SMILES: CC1(C)c2ccccc2-c2ccc(N(c3cccc(-c4ccccc4-c4ccccc4)c3)c3ccc4ccc5cc6oc7ccccc7c6cc5c4c3)cc21 ; c1ccc(-c2ccc(N(c3ccc4ccc5cc6oc7ccccc7c6cc5c4c3)c3ccc4ccc5c6ccccc6[se]c5c4c3)cc2)cc1 ; [2H]c1c([2H])c([2H])c(N(c2c([2H])c([2H])c3c([2H])c([2H])c4c([2H])c5oc6c([2H])c([2H])c([2H])c([2H])c6c5c([2H])c4c3c2[2H])c2c([2H])c([2H])c([2H])c3oc4c([2H])c([2H])c([2H])c([2H])c4c23)c([2H])c1[2H]]

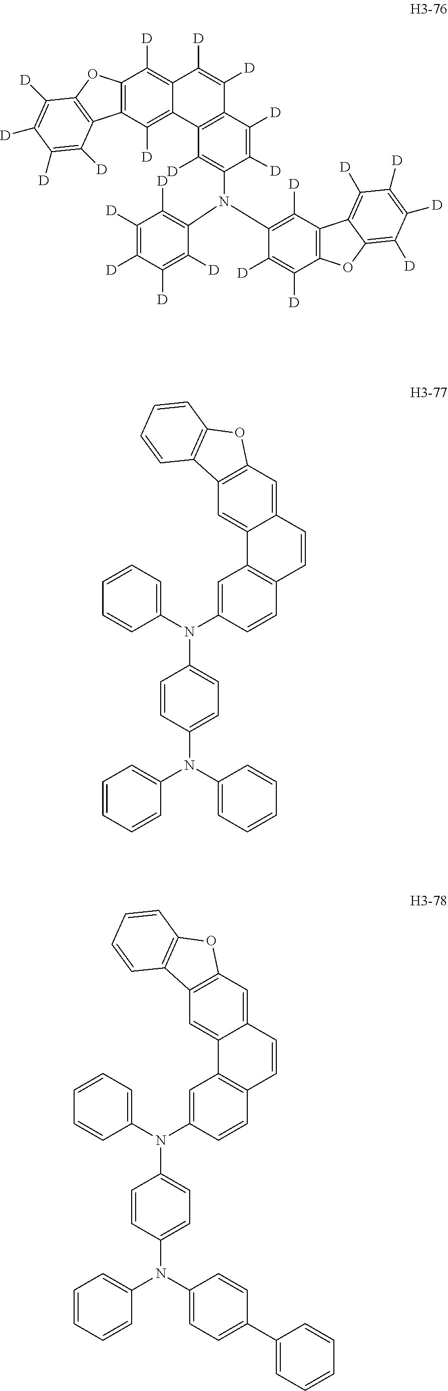 [SMILES: [2H]c1c([2H])c([2H])c(N(c2c([2H])c([2H])c3oc4c([2H])c([2H])c([2H])c([2H])c4c3c2[2H])c2c([2H])c([2H])c3c([2H])c([2H])c4c([2H])c5oc6c([2H])c([2H])c([2H])c([2H])c6c5c([2H])c4c3c2[2H])c([2H])c1[2H] ; c1ccc(N(c2ccccc2)c2ccc(N(c3ccccc3)c3ccc4ccc5cc6oc7ccccc7c6cc5c4c3)cc2)cc1 ; c1ccc(-c2ccc(N(c3ccccc3)c3ccc(N(c4ccccc4)c4ccc5ccc6cc7oc8ccccc8c7cc6c5c4)cc3)cc2)cc1]

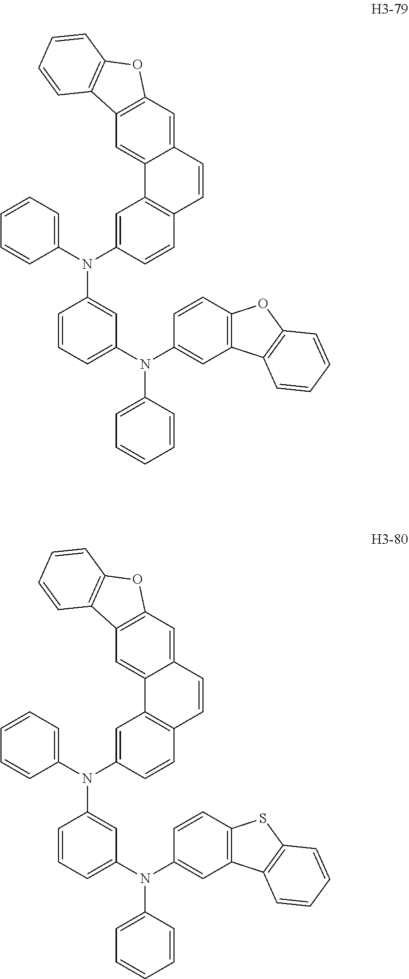 [SMILES: c1ccc(N(c2cccc(N(c3ccccc3)c3ccc4oc5ccccc5c4c3)c2)c2ccc3ccc4cc5oc6ccccc6c5cc4c3c2)cc1 ; c1ccc(N(c2cccc(N(c3ccccc3)c3ccc4sc5ccccc5c4c3)c2)c2ccc3ccc4cc5oc6ccccc6c5cc4c3c2)cc1]

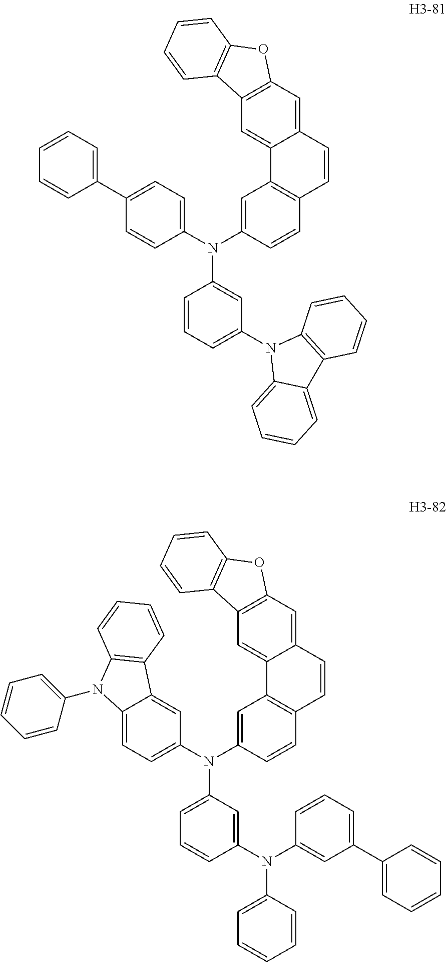 [SMILES: c1ccc(-c2ccc(N(c3cccc(-n4c5ccccc5c5ccccc54)c3)c3ccc4ccc5cc6oc7ccccc7c6cc5c4c3)cc2)cc1 ; c1ccc(-c2cccc(N(c3ccccc3)c3cccc(N(c4ccc5ccc6cc7oc8ccccc8c7cc6c5c4)c4ccc5c(c4)c4ccccc4n5-c4ccccc4)c3)c2)cc1]

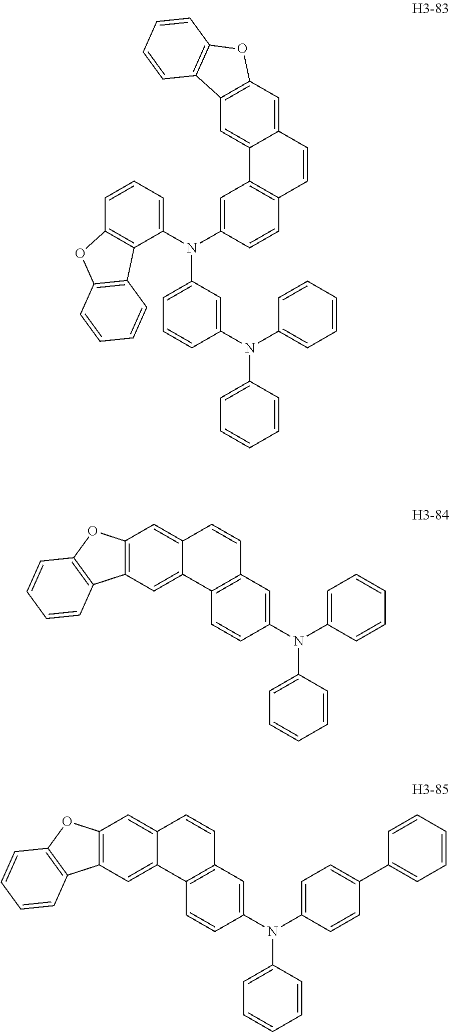 [SMILES: c1ccc(N(c2ccccc2)c2cccc(N(c3ccc4ccc5cc6oc7ccccc7c6cc5c4c3)c3cccc4oc5ccccc5c34)c2)cc1 ; c1ccc(N(c2ccccc2)c2ccc3c(ccc4cc5oc6ccccc6c5cc43)c2)cc1 ; c1ccc(-c2ccc(N(c3ccccc3)c3ccc4c(ccc5cc6oc7ccccc7c6cc54)c3)cc2)cc1]

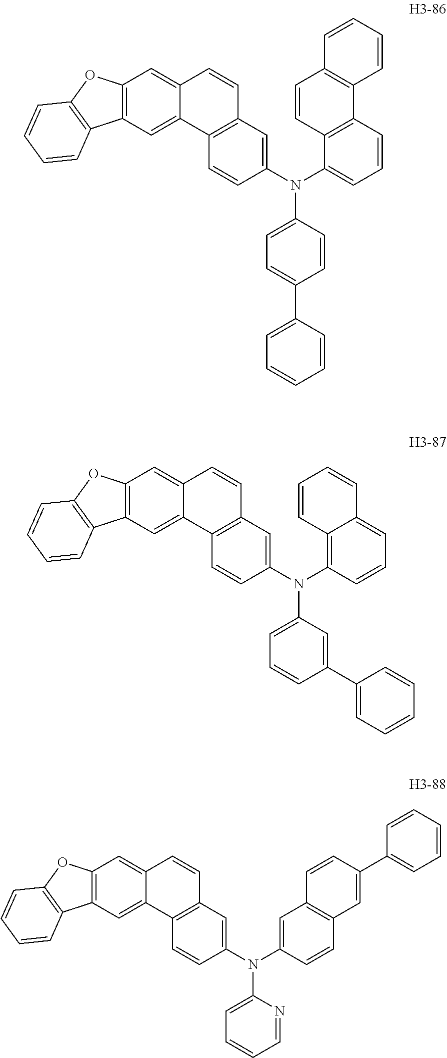 [SMILES: c1ccc(-c2ccc(N(c3ccc4c(ccc5cc6oc7ccccc7c6cc54)c3)c3cccc4c3ccc3ccccc34)cc2)cc1 ; c1ccc(-c2cccc(N(c3ccc4c(ccc5cc6oc7ccccc7c6cc54)c3)c3cccc4ccccc34)c2)cc1 ; c1ccc(-c2ccc3cc(N(c4ccc5c(ccc6cc7oc8ccccc8c7cc65)c4)c4ccccn4)ccc3c2)cc1]

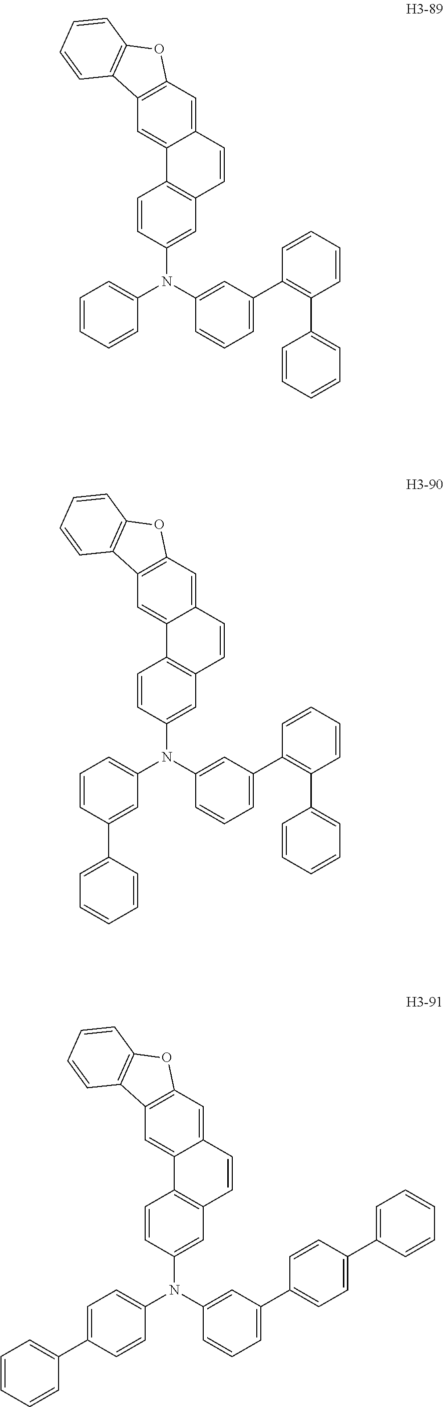 [SMILES: c1ccc(-c2ccccc2-c2cccc(N(c3ccccc3)c3ccc4c(ccc5cc6oc7ccccc7c6cc54)c3)c2)cc1 ; c1ccc(-c2cccc(N(c3cccc(-c4ccccc4-c4ccccc4)c3)c3ccc4c(ccc5cc6oc7ccccc7c6cc54)c3)c2)cc1 ; c1ccc(-c2ccc(-c3cccc(N(c4ccc(-c5ccccc5)cc4)c4ccc5c(ccc6cc7oc8ccccc8c7cc65)c4)c3)cc2)cc1]

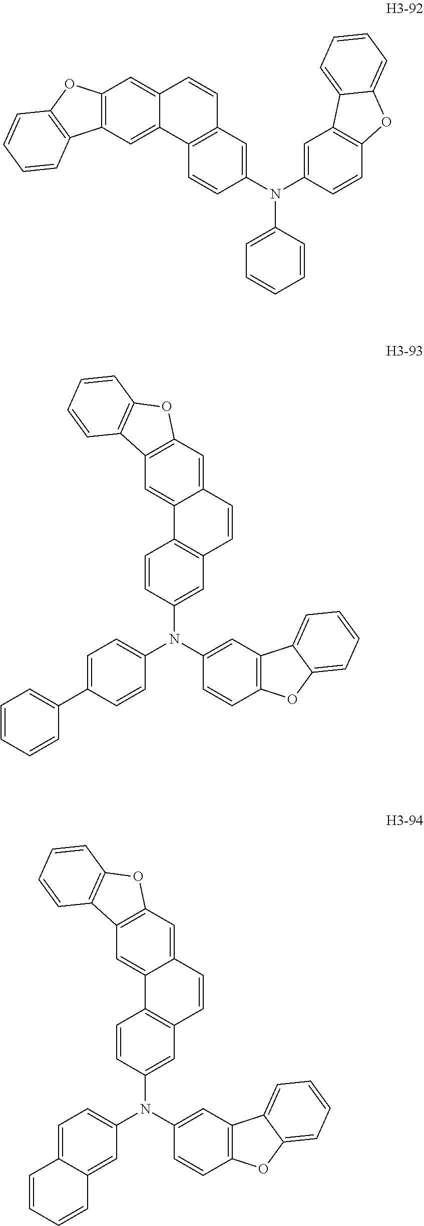 [SMILES: c1ccc(N(c2ccc3c(ccc4cc5oc6ccccc6c5cc43)c2)c2ccc3oc4ccccc4c3c2)cc1 ; c1ccc(-c2ccc(N(c3ccc4c(ccc5cc6oc7ccccc7c6cc54)c3)c3ccc4oc5ccccc5c4c3)cc2)cc1 ; c1ccc2cc(N(c3ccc4c(ccc5cc6oc7ccccc7c6cc54)c3)c3ccc4oc5ccccc5c4c3)ccc2c1]

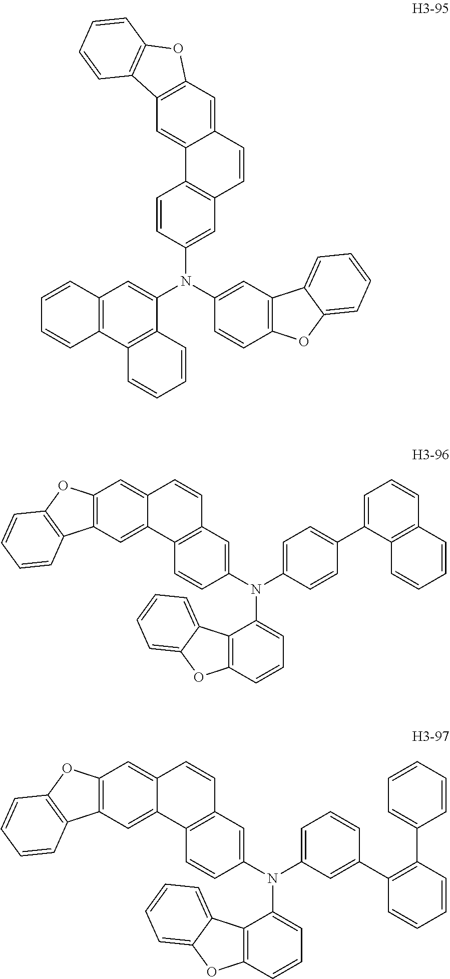 [SMILES: c1ccc2c(c1)cc(N(c1ccc3c(ccc4cc5oc6ccccc6c5cc43)c1)c1ccc3oc4ccccc4c3c1)c1ccccc12 ; c1ccc2c(-c3ccc(N(c4ccc5c(ccc6cc7oc8ccccc8c7cc65)c4)c4cccc5oc6ccccc6c45)cc3)cccc2c1 ; c1ccc(-c2ccccc2-c2cccc(N(c3ccc4c(ccc5cc6oc7ccccc7c6cc54)c3)c3cccc4oc5ccccc5c34)c2)cc1]

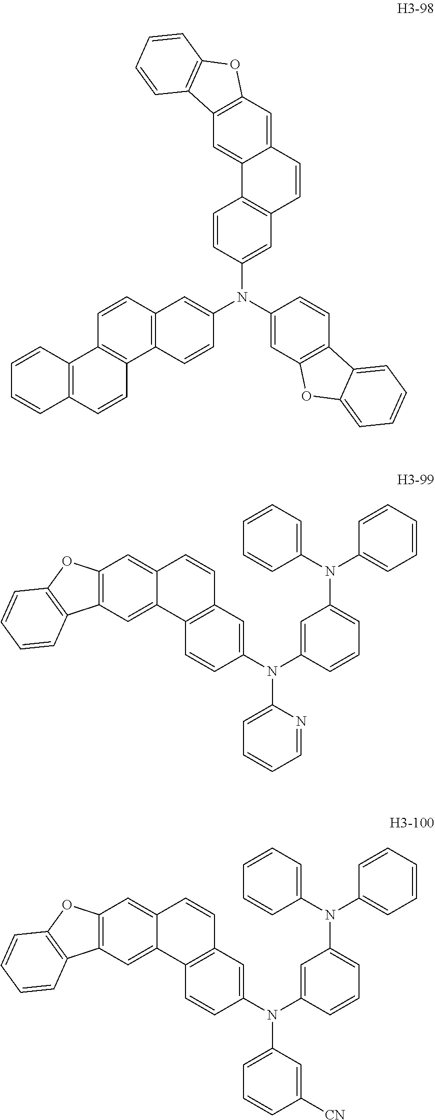 [SMILES: c1ccc2c(c1)ccc1c3ccc(N(c4ccc5c(ccc6cc7oc8ccccc8c7cc65)c4)c4ccc5c(c4)oc4ccccc45)cc3ccc21 ; c1ccc(N(c2ccccc2)c2cccc(N(c3ccc4c(ccc5cc6oc7ccccc7c6cc54)c3)c3ccccn3)c2)cc1 ; N#Cc1cccc(N(c2cccc(N(c3ccccc3)c3ccccc3)c2)c2ccc3c(ccc4cc5oc6ccccc6c5cc43)c2)c1]

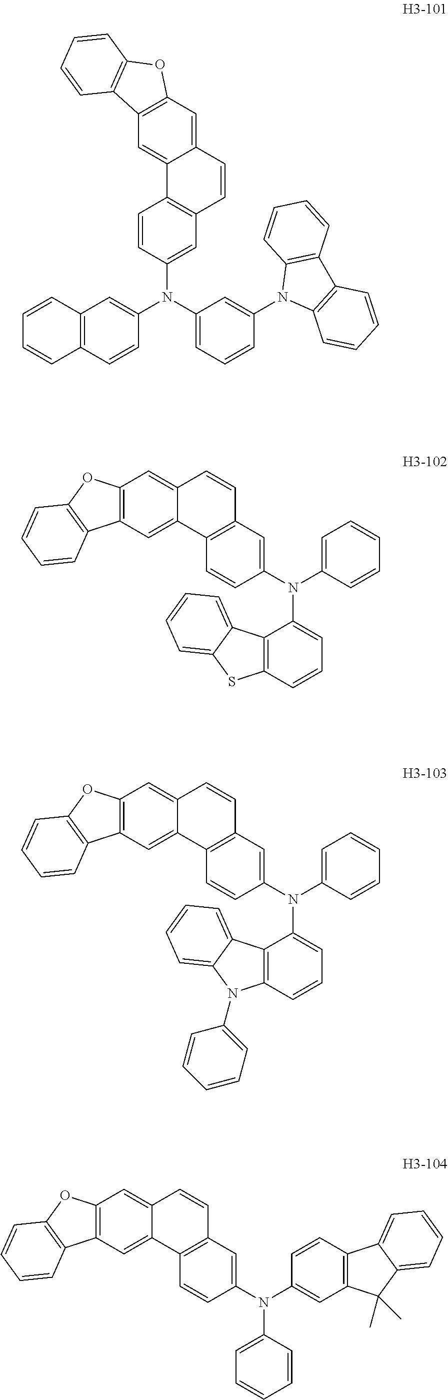 [SMILES: c1cc(N(c2ccc3ccccc3c2)c2ccc3c(ccc4cc5oc6ccccc6c5cc43)c2)cc(-n2c3ccccc3c3ccccc32)c1 ; c1ccc(N(c2ccc3c(ccc4cc5oc6ccccc6c5cc43)c2)c2cccc3sc4ccccc4c23)cc1 ; c1ccc(N(c2ccc3c(ccc4cc5oc6ccccc6c5cc43)c2)c2cccc3c2c2ccccc2n3-c2ccccc2)cc1 ; CC1(C)c2ccccc2-c2ccc(N(c3ccccc3)c3ccc4c(ccc5cc6oc7ccccc7c6cc54)c3)cc21]

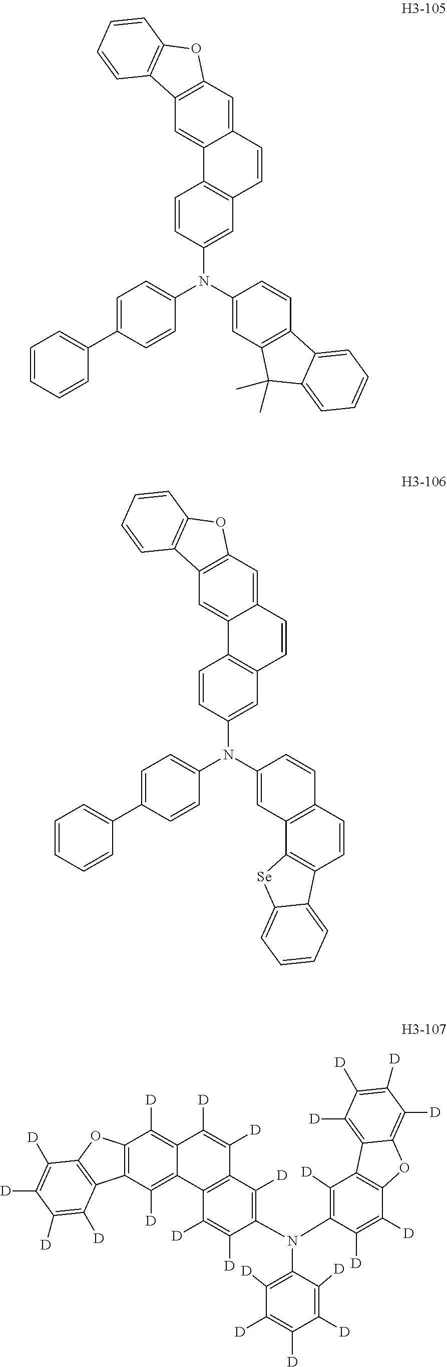 [SMILES: CC1(C)c2ccccc2-c2ccc(N(c3ccc(-c4ccccc4)cc3)c3ccc4c(ccc5cc6oc7ccccc7c6cc54)c3)cc21 ; c1ccc(-c2ccc(N(c3ccc4c(ccc5cc6oc7ccccc7c6cc54)c3)c3ccc4ccc5c6ccccc6[se]c5c4c3)cc2)cc1 ; [2H]c1c([2H])c([2H])c(N(c2c([2H])c([2H])c3c(c2[2H])c([2H])c([2H])c2c([2H])c4oc5c([2H])c([2H])c([2H])c([2H])c5c4c([2H])c23)c2c([2H])c([2H])c3oc4c([2H])c([2H])c([2H])c([2H])c4c3c2[2H])c([2H])c1[2H]]

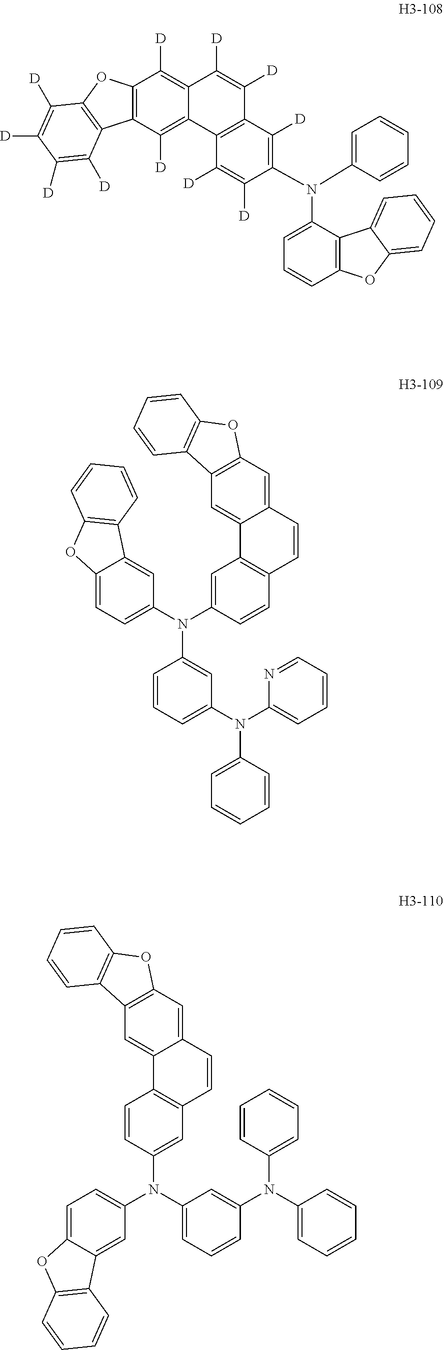 [SMILES: [2H]c1c([2H])c([2H])c2c(oc3c([2H])c4c([2H])c([2H])c5c([2H])c(N(c6ccccc6)c6cccc7oc8ccccc8c67)c([2H])c([2H])c5c4c([2H])c32)c1[2H] ; c1ccc(N(c2cccc(N(c3ccc4ccc5cc6oc7ccccc7c6cc5c4c3)c3ccc4oc5ccccc5c4c3)c2)c2ccccn2)cc1 ; c1ccc(N(c2ccccc2)c2cccc(N(c3ccc4c(ccc5cc6oc7ccccc7c6cc54)c3)c3ccc4oc5ccccc5c4c3)c2)cc1]

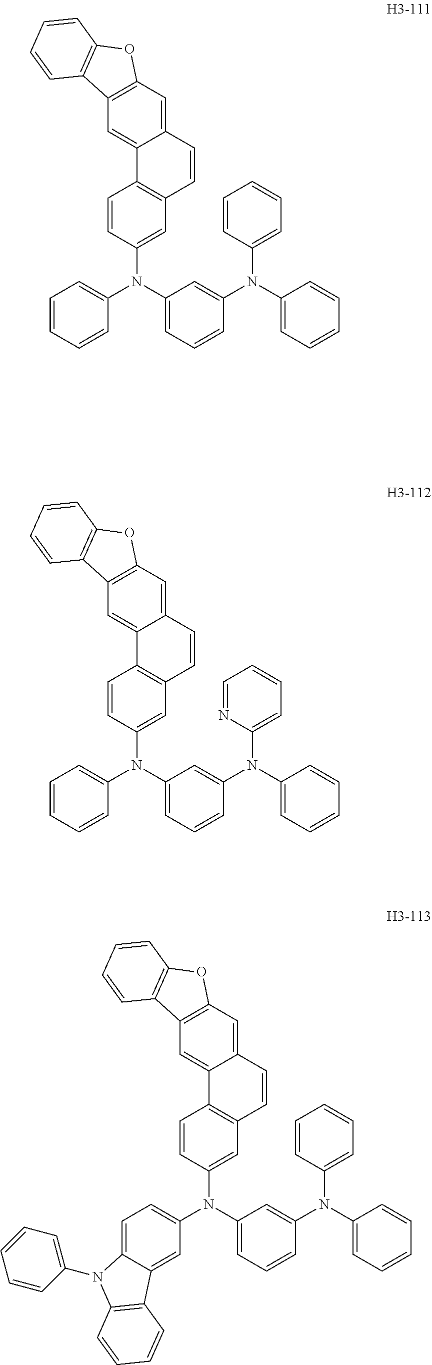 [SMILES: c1ccc(N(c2ccccc2)c2cccc(N(c3ccccc3)c3ccc4c(ccc5cc6oc7ccccc7c6cc54)c3)c2)cc1 ; c1ccc(N(c2cccc(N(c3ccccc3)c3ccccn3)c2)c2ccc3c(ccc4cc5oc6ccccc6c5cc43)c2)cc1 ; c1ccc(N(c2ccccc2)c2cccc(N(c3ccc4c(ccc5cc6oc7ccccc7c6cc54)c3)c3ccc4c(c3)c3ccccc3n4-c3ccccc3)c2)cc1]

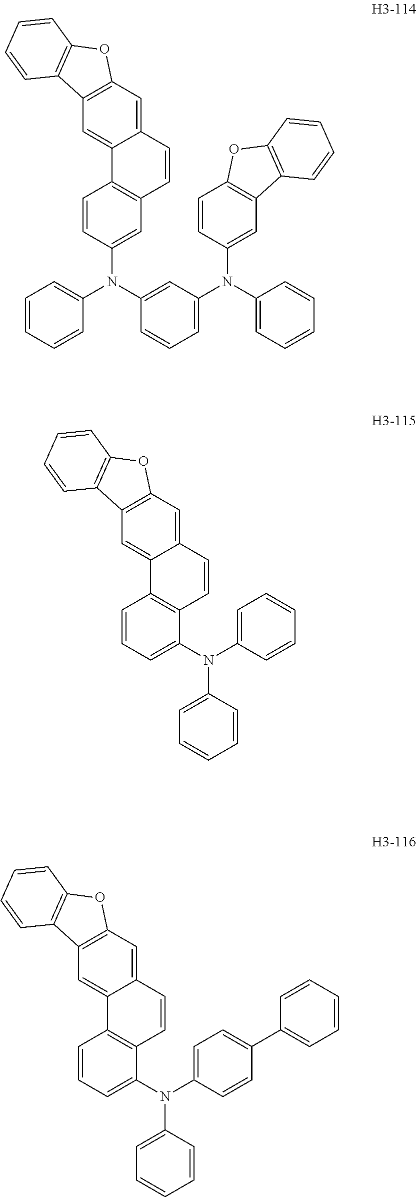 [SMILES: c1ccc(N(c2cccc(N(c3ccccc3)c3ccc4oc5ccccc5c4c3)c2)c2ccc3c(ccc4cc5oc6ccccc6c5cc43)c2)cc1 ; c1ccc(N(c2ccccc2)c2cccc3c2ccc2cc4oc5ccccc5c4cc23)cc1 ; c1ccc(-c2ccc(N(c3ccccc3)c3cccc4c3ccc3cc5oc6ccccc6c5cc34)cc2)cc1]

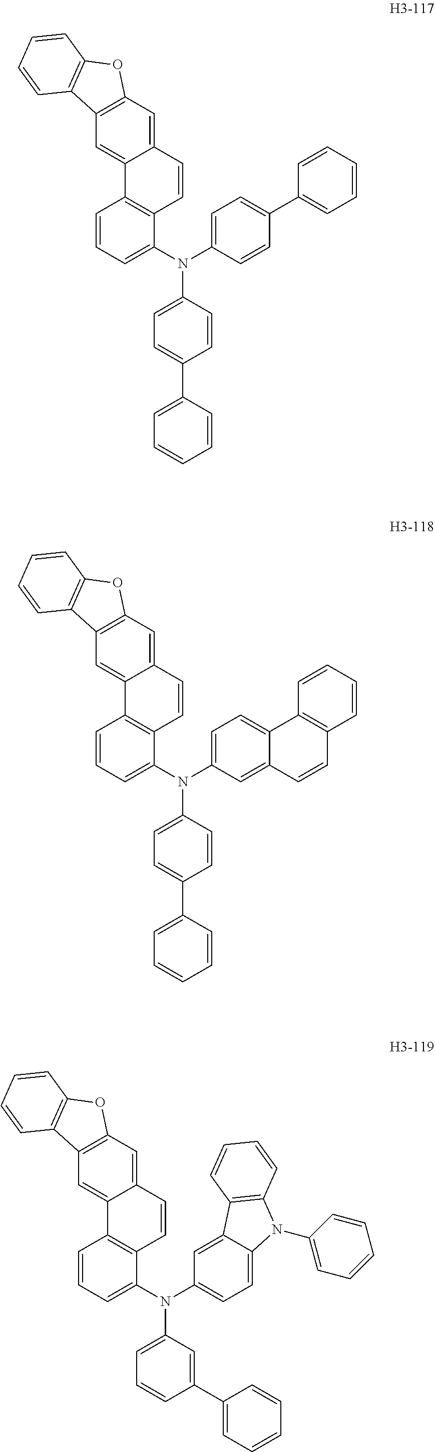 [SMILES: c1ccc(-c2ccc(N(c3ccc(-c4ccccc4)cc3)c3cccc4c3ccc3cc5oc6ccccc6c5cc34)cc2)cc1 ; c1ccc(-c2ccc(N(c3ccc4c(ccc5ccccc54)c3)c3cccc4c3ccc3cc5oc6ccccc6c5cc34)cc2)cc1 ; c1ccc(-c2cccc(N(c3ccc4c(c3)c3ccccc3n4-c3ccccc3)c3cccc4c3ccc3cc5oc6ccccc6c5cc34)c2)cc1]

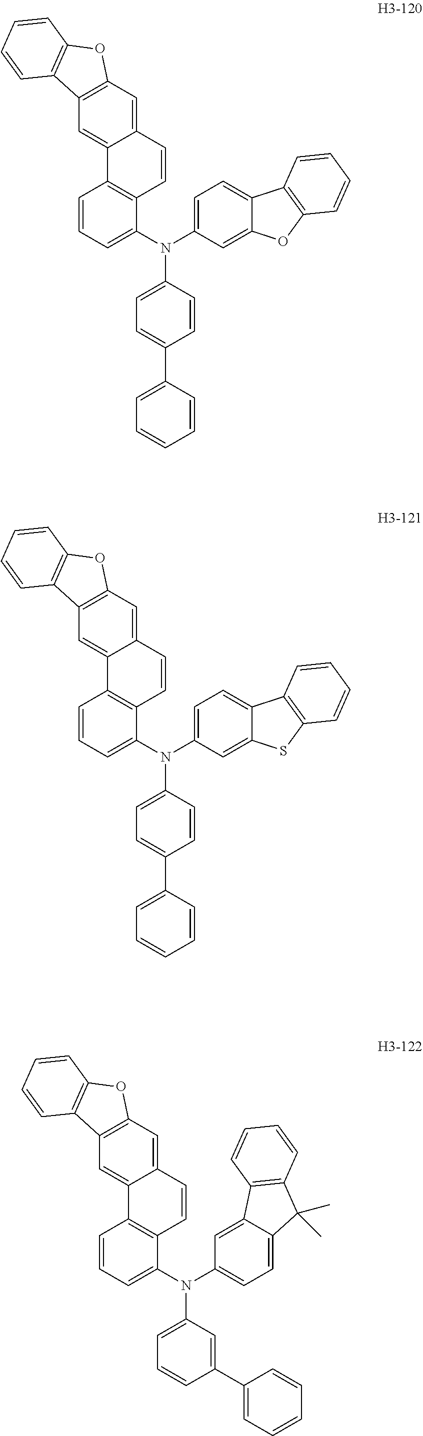 [SMILES: c1ccc(-c2ccc(N(c3ccc4c(c3)oc3ccccc34)c3cccc4c3ccc3cc5oc6ccccc6c5cc34)cc2)cc1 ; c1ccc(-c2ccc(N(c3ccc4c(c3)sc3ccccc34)c3cccc4c3ccc3cc5oc6ccccc6c5cc34)cc2)cc1 ; CC1(C)c2ccccc2-c2cc(N(c3cccc(-c4ccccc4)c3)c3cccc4c3ccc3cc5oc6ccccc6c5cc34)ccc21]

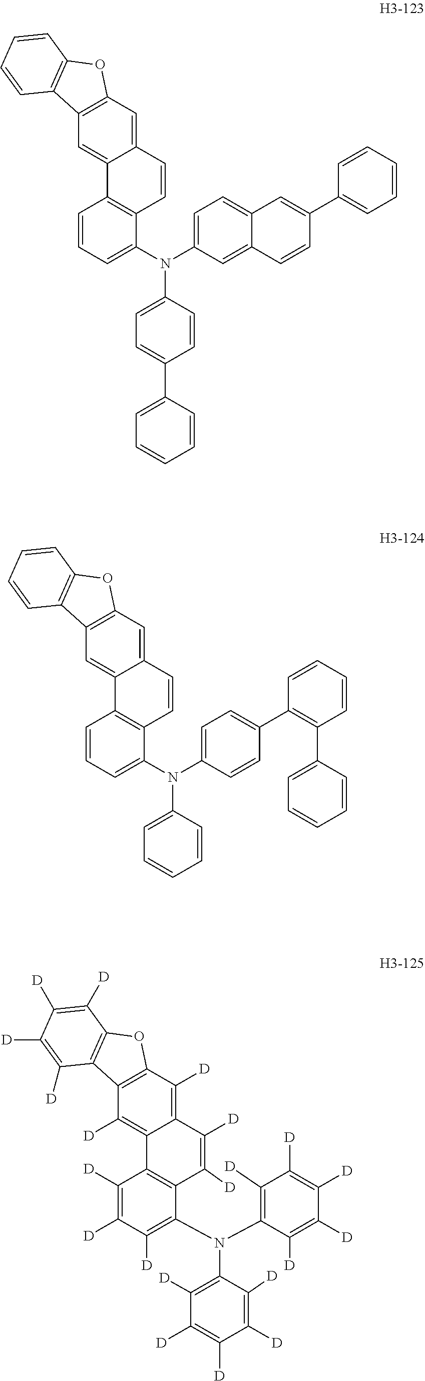 [SMILES: c1ccc(-c2ccc(N(c3ccc4cc(-c5ccccc5)ccc4c3)c3cccc4c3ccc3cc5oc6ccccc6c5cc34)cc2)cc1 ; c1ccc(-c2ccccc2-c2ccc(N(c3ccccc3)c3cccc4c3ccc3cc5oc6ccccc6c5cc34)cc2)cc1 ; [2H]c1c([2H])c([2H])c(N(c2c([2H])c([2H])c([2H])c([2H])c2[2H])c2c([2H])c([2H])c([2H])c3c2c([2H])c([2H])c2c([2H])c4oc5c([2H])c([2H])c([2H])c([2H])c5c4c([2H])c23)c([2H])c1[2H]]

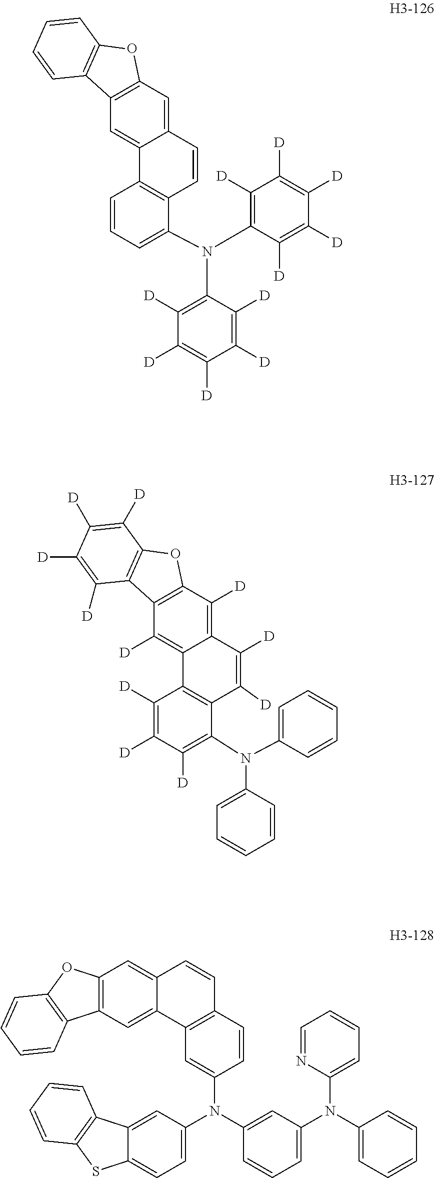 [SMILES: [2H]c1c([2H])c([2H])c(N(c2c([2H])c([2H])c([2H])c([2H])c2[2H])c2cccc3c2ccc2cc4oc5ccccc5c4cc23)c([2H])c1[2H] ; [2H]c1c([2H])c([2H])c2c(oc3c([2H])c4c([2H])c([2H])c5c(N(c6ccccc6)c6ccccc6)c([2H])c([2H])c([2H])c5c4c([2H])c32)c1[2H] ; c1ccc(N(c2cccc(N(c3ccc4ccc5cc6oc7ccccc7c6cc5c4c3)c3ccc4sc5ccccc5c4c3)c2)c2ccccn2)cc1]

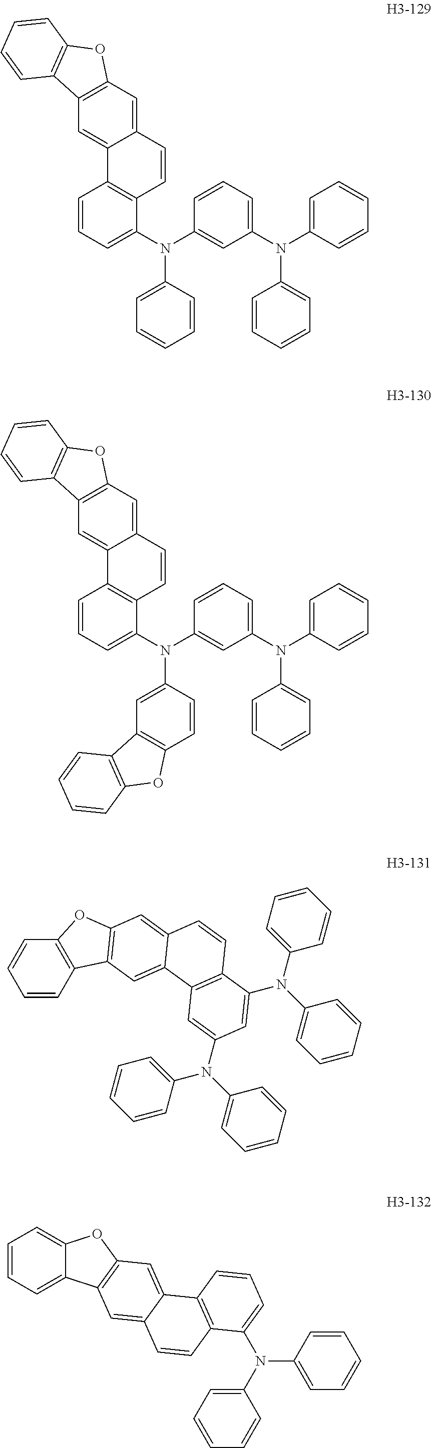 [SMILES: c1ccc(N(c2ccccc2)c2cccc(N(c3ccccc3)c3cccc4c3ccc3cc5oc6ccccc6c5cc34)c2)cc1 ; c1ccc(N(c2ccccc2)c2cccc(N(c3ccc4oc5ccccc5c4c3)c3cccc4c3ccc3cc5oc6ccccc6c5cc34)c2)cc1 ; c1ccc(N(c2ccccc2)c2cc(N(c3ccccc3)c3ccccc3)c3ccc4cc5oc6ccccc6c5cc4c3c2)cc1 ; c1ccc(N(c2ccccc2)c2cccc3c2ccc2cc4c(cc23)oc2ccccc24)cc1]

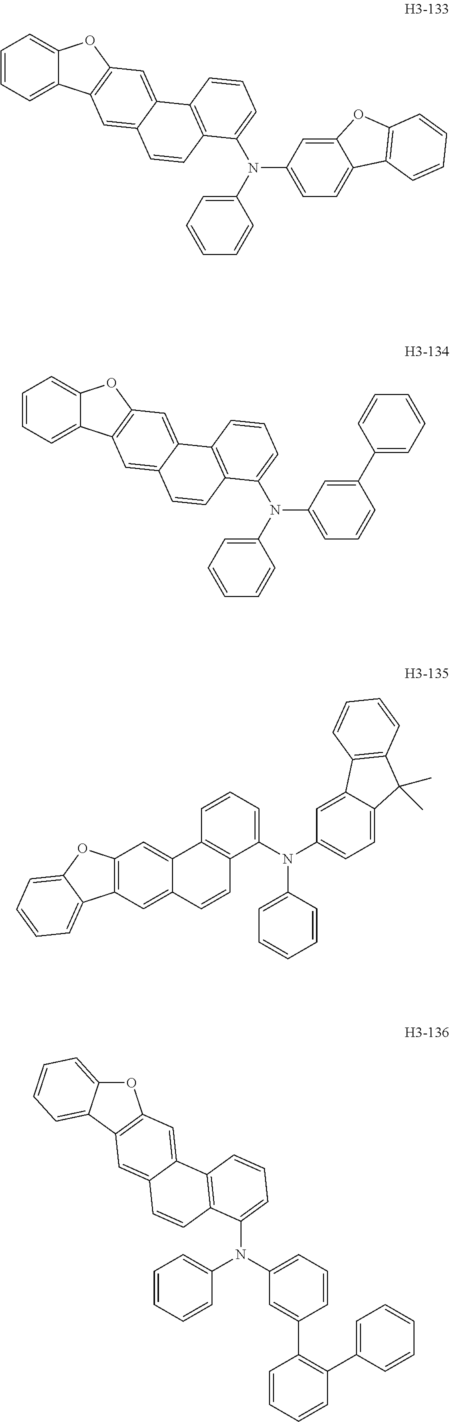 [SMILES: c1ccc(N(c2ccc3c(c2)oc2ccccc23)c2cccc3c2ccc2cc4c(cc23)oc2ccccc24)cc1 ; c1ccc(-c2cccc(N(c3ccccc3)c3cccc4c3ccc3cc5c(cc34)oc3ccccc35)c2)cc1 ; CC1(C)c2ccccc2-c2cc(N(c3ccccc3)c3cccc4c3ccc3cc5c(cc34)oc3ccccc35)ccc21 ; c1ccc(-c2ccccc2-c2cccc(N(c3ccccc3)c3cccc4c3ccc3cc5c(cc34)oc3ccccc35)c2)cc1]

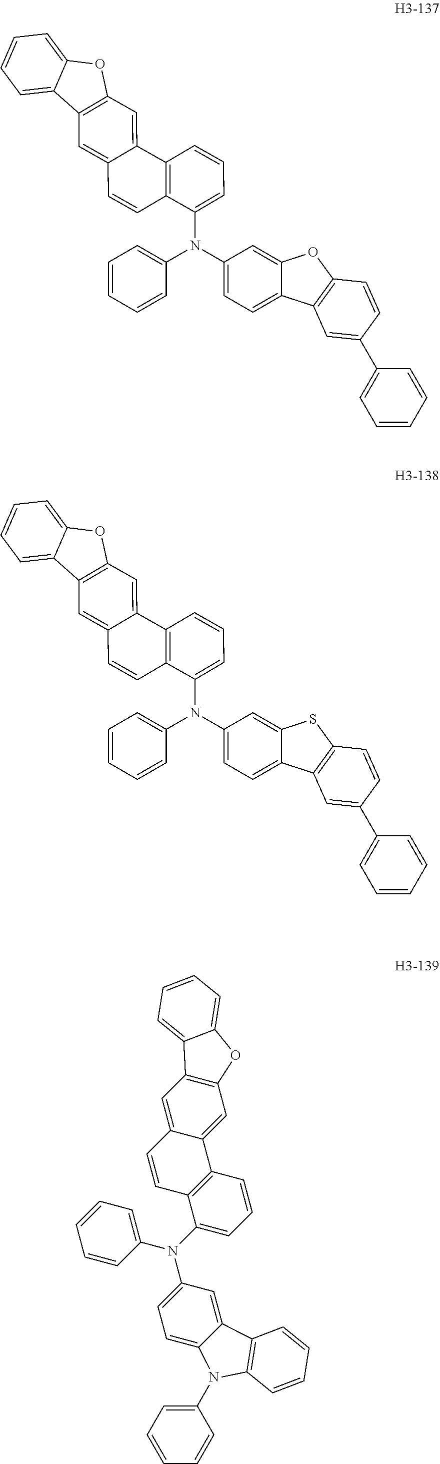 [SMILES: c1ccc(-c2ccc3oc4cc(N(c5ccccc5)c5cccc6c5ccc5cc7c(cc56)oc5ccccc57)ccc4c3c2)cc1 ; c1ccc(-c2ccc3sc4cc(N(c5ccccc5)c5cccc6c5ccc5cc7c(cc56)oc5ccccc57)ccc4c3c2)cc1 ; c1ccc(N(c2ccc3c(c2)c2ccccc2n3-c2ccccc2)c2cccc3c2ccc2cc4c(cc23)oc2ccccc24)cc1]

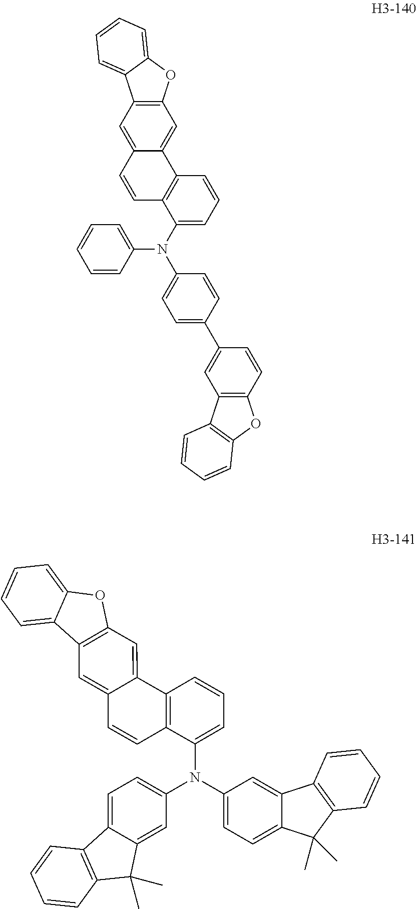 [SMILES: c1ccc(N(c2ccc(-c3ccc4oc5ccccc5c4c3)cc2)c2cccc3c2ccc2cc4c(cc23)oc2ccccc24)cc1 ; CC1(C)c2ccccc2-c2cc(N(c3ccc4c(c3)C(C)(C)c3ccccc3-4)c3cccc4c3ccc3cc5c(cc34)oc3ccccc35)ccc21]

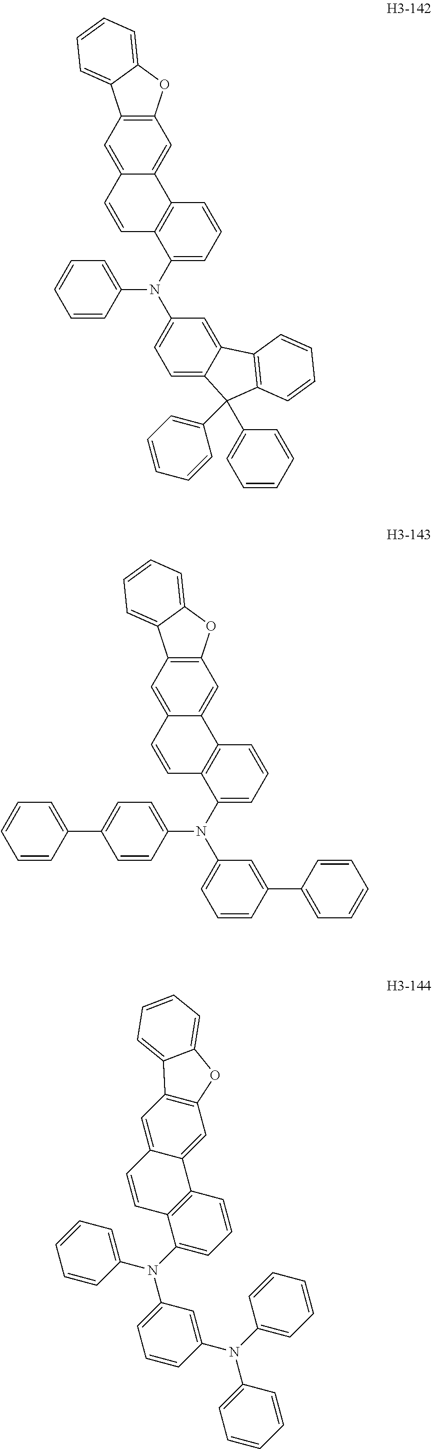 [SMILES: c1ccc(N(c2ccc3c(c2)-c2ccccc2C3(c2ccccc2)c2ccccc2)c2cccc3c2ccc2cc4c(cc23)oc2ccccc24)cc1 ; c1ccc(-c2ccc(N(c3cccc(-c4ccccc4)c3)c3cccc4c3ccc3cc5c(cc34)oc3ccccc35)cc2)cc1 ; c1ccc(N(c2ccccc2)c2cccc(N(c3ccccc3)c3cccc4c3ccc3cc5c(cc34)oc3ccccc35)c2)cc1]

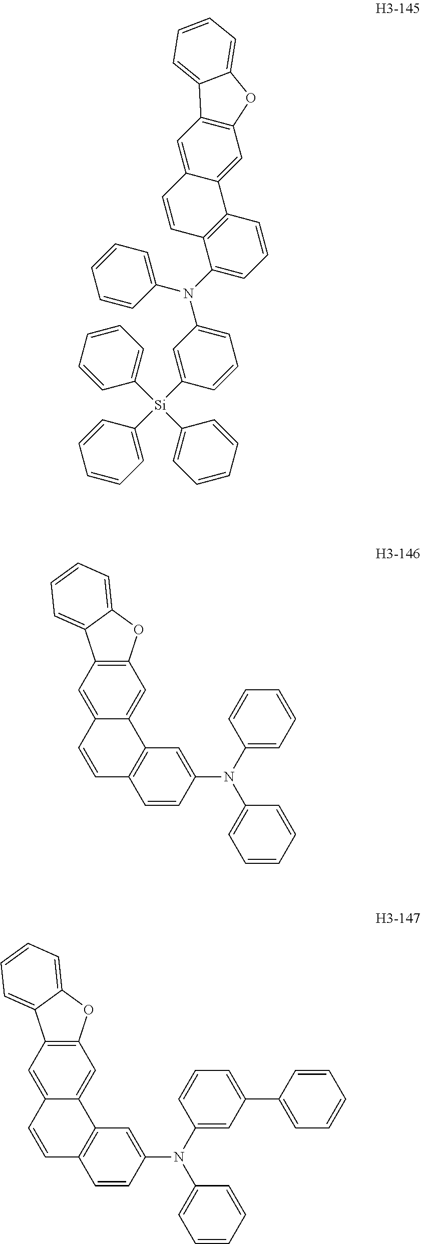 [SMILES: c1ccc(N(c2cccc([Si](c3ccccc3)(c3ccccc3)c3ccccc3)c2)c2cccc3c2ccc2cc4c(cc23)oc2ccccc24)cc1 ; c1ccc(N(c2ccccc2)c2ccc3ccc4cc5c(cc4c3c2)oc2ccccc25)cc1 ; c1ccc(-c2cccc(N(c3ccccc3)c3ccc4ccc5cc6c(cc5c4c3)oc3ccccc36)c2)cc1]

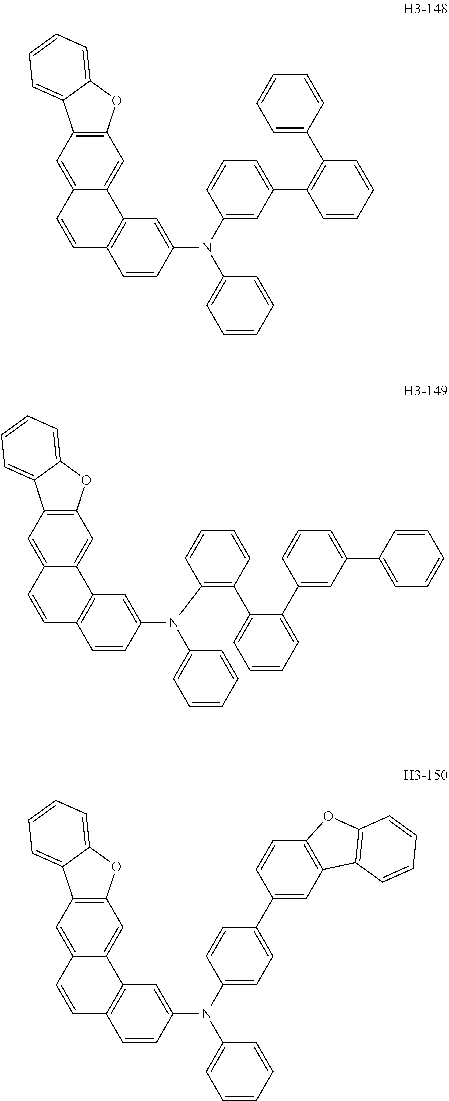 [SMILES: c1ccc(-c2ccccc2-c2cccc(N(c3ccccc3)c3ccc4ccc5cc6c(cc5c4c3)oc3ccccc36)c2)cc1 ; c1ccc(-c2cccc(-c3ccccc3-c3ccccc3N(c3ccccc3)c3ccc4ccc5cc6c(cc5c4c3)oc3ccccc36)c2)cc1 ; c1ccc(N(c2ccc(-c3ccc4oc5ccccc5c4c3)cc2)c2ccc3ccc4cc5c(cc4c3c2)oc2ccccc25)cc1]

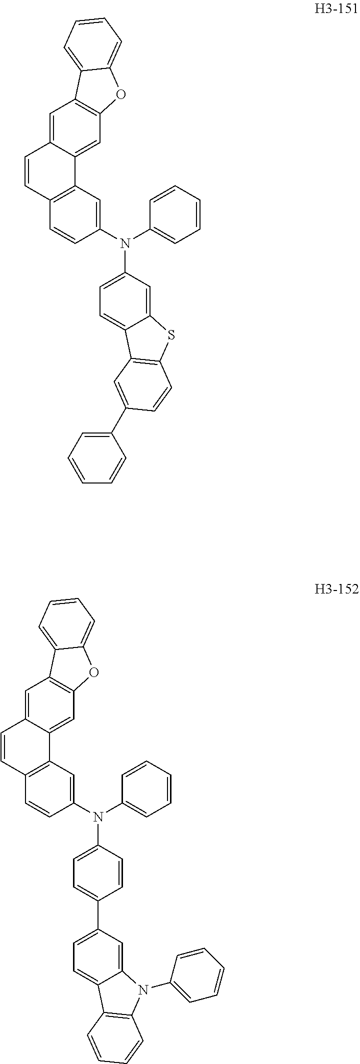 [SMILES: c1ccc(-c2ccc3sc4cc(N(c5ccccc5)c5ccc6ccc7cc8c(cc7c6c5)oc5ccccc58)ccc4c3c2)cc1 ; c1ccc(N(c2ccc(-c3ccc4c5ccccc5n(-c5ccccc5)c4c3)cc2)c2ccc3ccc4cc5c(cc4c3c2)oc2ccccc25)cc1]

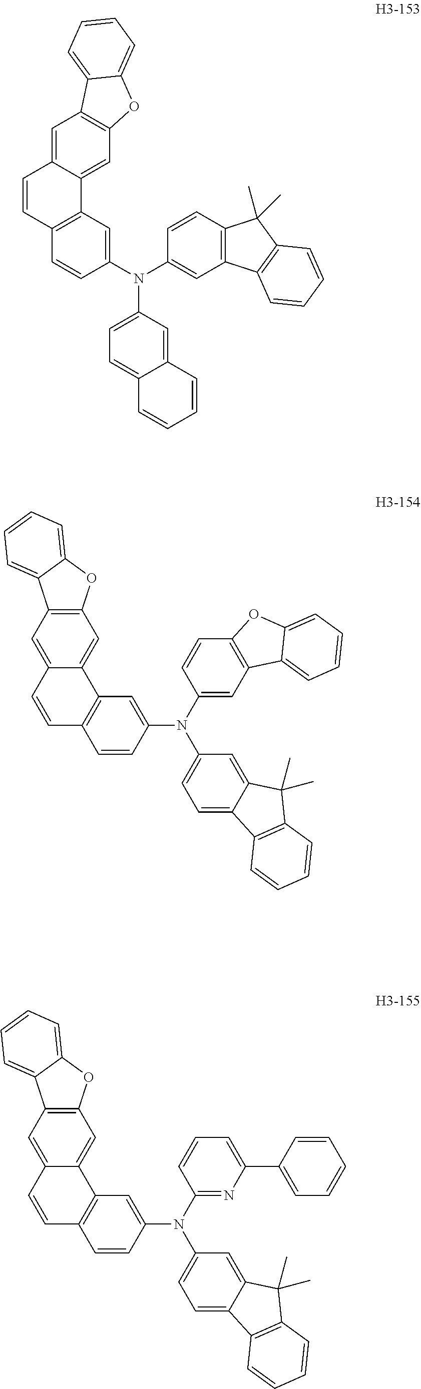 [SMILES: CC1(C)c2ccccc2-c2cc(N(c3ccc4ccccc4c3)c3ccc4ccc5cc6c(cc5c4c3)oc3ccccc36)ccc21 ; CC1(C)c2ccccc2-c2ccc(N(c3ccc4ccc5cc6c(cc5c4c3)oc3ccccc36)c3ccc4oc5ccccc5c4c3)cc21 ; CC1(C)c2ccccc2-c2ccc(N(c3ccc4ccc5cc6c(cc5c4c3)oc3ccccc36)c3cccc(-c4ccccc4)n3)cc21]

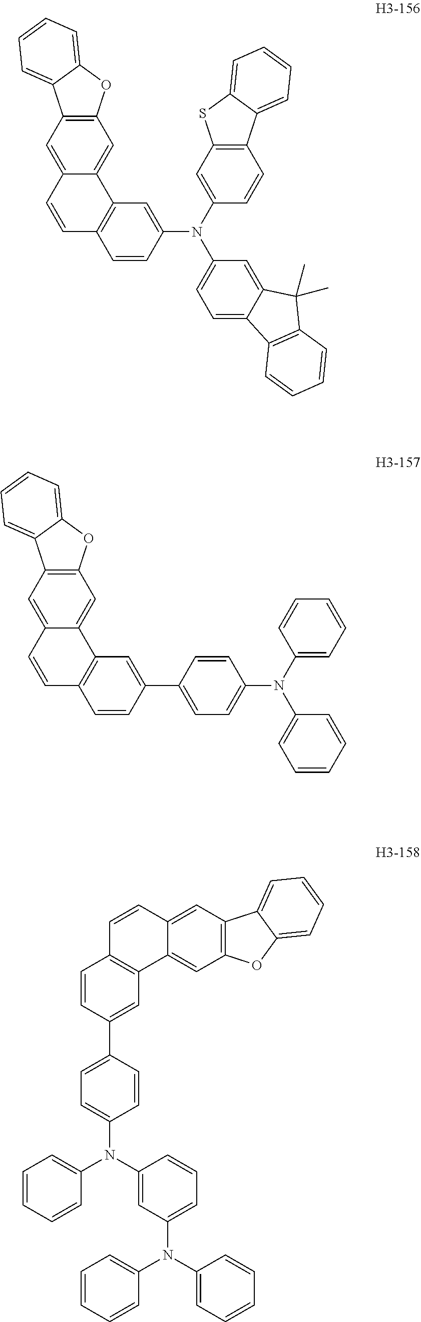 [SMILES: CC1(C)c2ccccc2-c2ccc(N(c3ccc4c(c3)sc3ccccc34)c3ccc4ccc5cc6c(cc5c4c3)oc3ccccc36)cc21 ; c1ccc(N(c2ccccc2)c2ccc(-c3ccc4ccc5cc6c(cc5c4c3)oc3ccccc36)cc2)cc1 ; c1ccc(N(c2ccccc2)c2cccc(N(c3ccccc3)c3ccc(-c4ccc5ccc6cc7c(cc6c5c4)oc4ccccc47)cc3)c2)cc1]

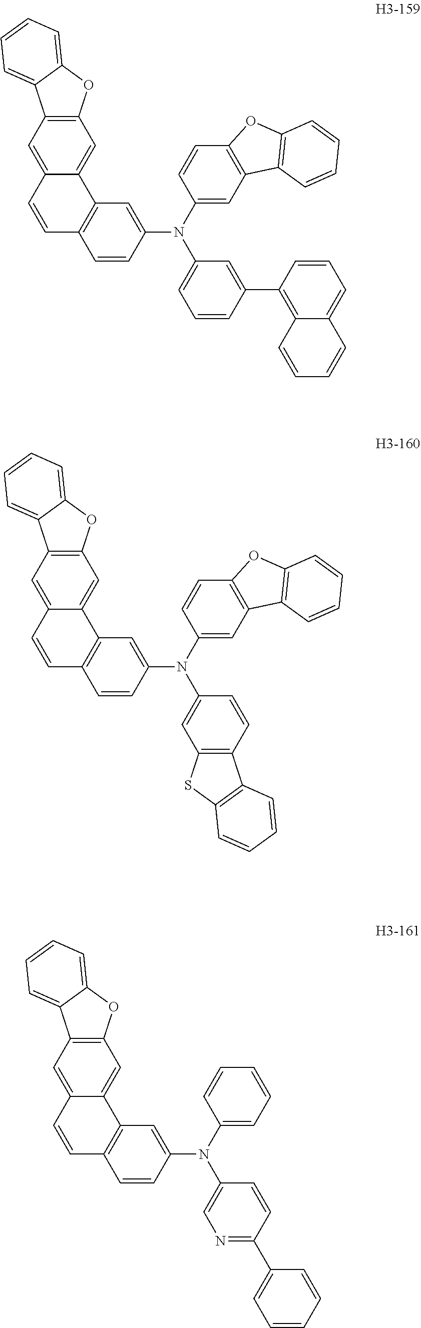 [SMILES: c1cc(-c2cccc3ccccc23)cc(N(c2ccc3ccc4cc5c(cc4c3c2)oc2ccccc25)c2ccc3oc4ccccc4c3c2)c1 ; c1ccc2c(c1)oc1ccc(N(c3ccc4c(c3)sc3ccccc34)c3ccc4ccc5cc6c(cc5c4c3)oc3ccccc36)cc12 ; c1ccc(-c2ccc(N(c3ccccc3)c3ccc4ccc5cc6c(cc5c4c3)oc3ccccc36)cn2)cc1]

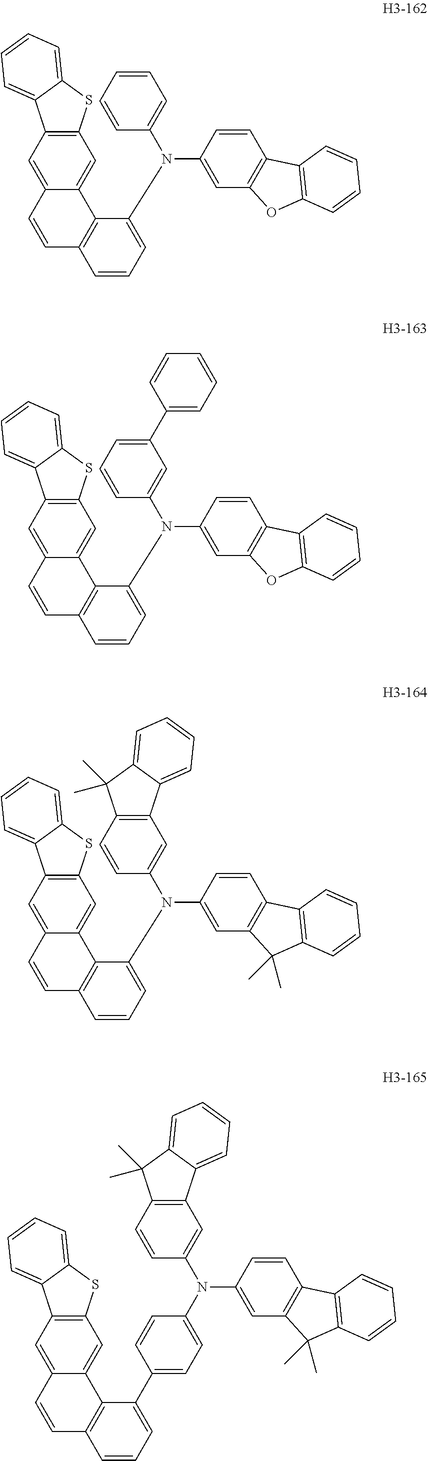 [SMILES: c1ccc(N(c2ccc3c(c2)oc2ccccc23)c2cccc3ccc4cc5c(cc4c23)sc2ccccc25)cc1 ; c1ccc(-c2cccc(N(c3ccc4c(c3)oc3ccccc34)c3cccc4ccc5cc6c(cc5c34)sc3ccccc36)c2)cc1 ; CC1(C)c2ccccc2-c2cc(N(c3ccc4c(c3)C(C)(C)c3ccccc3-4)c3cccc4ccc5cc6c(cc5c34)sc3ccccc36)ccc21 ; CC1(C)c2ccccc2-c2cc(N(c3ccc(-c4cccc5ccc6cc7c(cc6c45)sc4ccccc47)cc3)c3ccc4c(c3)C(C)(C)c3ccccc3-4)ccc21]

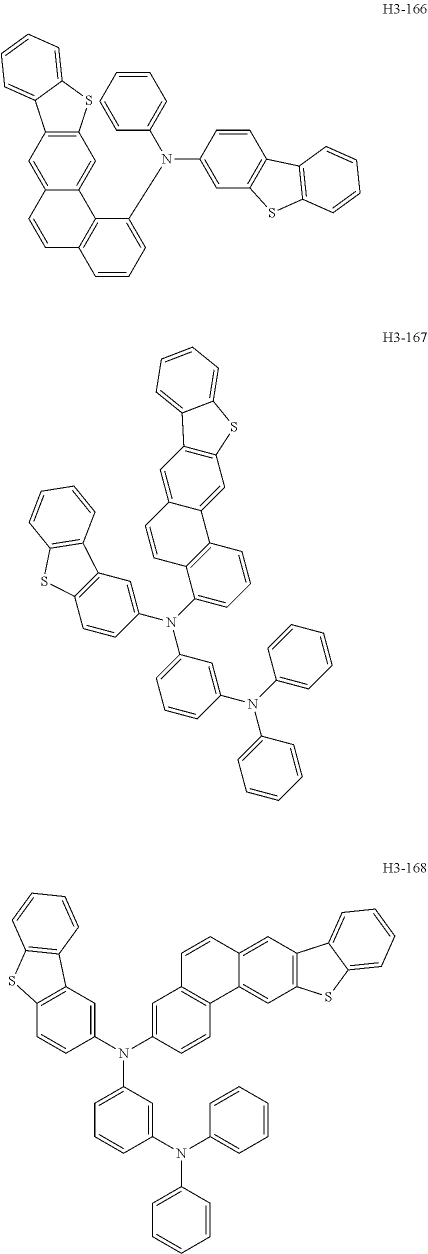 [SMILES: c1ccc(N(c2ccc3c(c2)sc2ccccc23)c2cccc3ccc4cc5c(cc4c23)sc2ccccc25)cc1 ; c1ccc(N(c2ccccc2)c2cccc(N(c3ccc4sc5ccccc5c4c3)c3cccc4c3ccc3cc5c(cc34)sc3ccccc35)c2)cc1 ; c1ccc(N(c2ccccc2)c2cccc(N(c3ccc4c(ccc5cc6c(cc54)sc4ccccc46)c3)c3ccc4sc5ccccc5c4c3)c2)cc1]

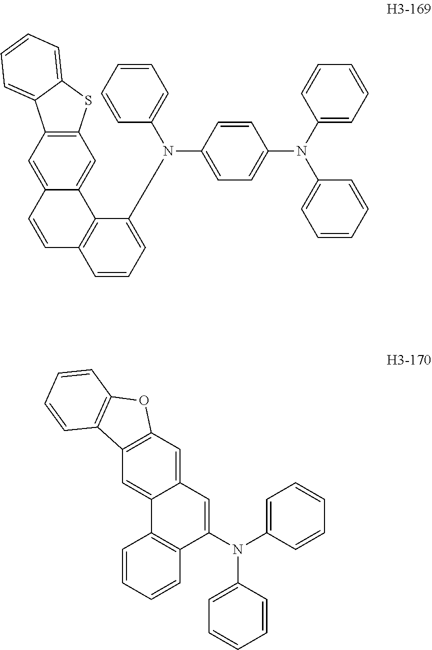 [SMILES: c1ccc(N(c2ccccc2)c2ccc(N(c3ccccc3)c3cccc4ccc5cc6c(cc5c34)sc3ccccc36)cc2)cc1 ; c1ccc(N(c2ccccc2)c2cc3cc4oc5ccccc5c4cc3c3ccccc23)cc1]

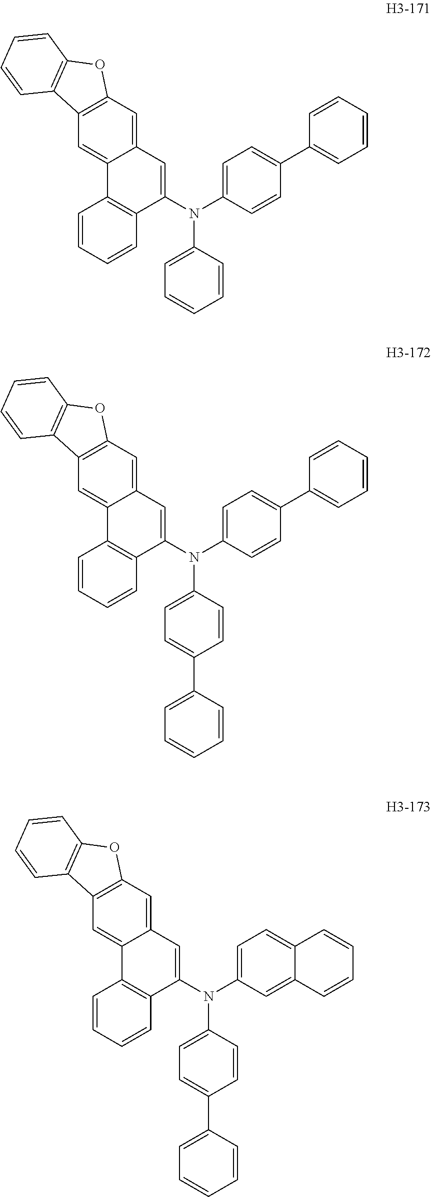 [SMILES: c1ccc(-c2ccc(N(c3ccccc3)c3cc4cc5oc6ccccc6c5cc4c4ccccc34)cc2)cc1 ; c1ccc(-c2ccc(N(c3ccc(-c4ccccc4)cc3)c3cc4cc5oc6ccccc6c5cc4c4ccccc34)cc2)cc1 ; c1ccc(-c2ccc(N(c3ccc4ccccc4c3)c3cc4cc5oc6ccccc6c5cc4c4ccccc34)cc2)cc1]

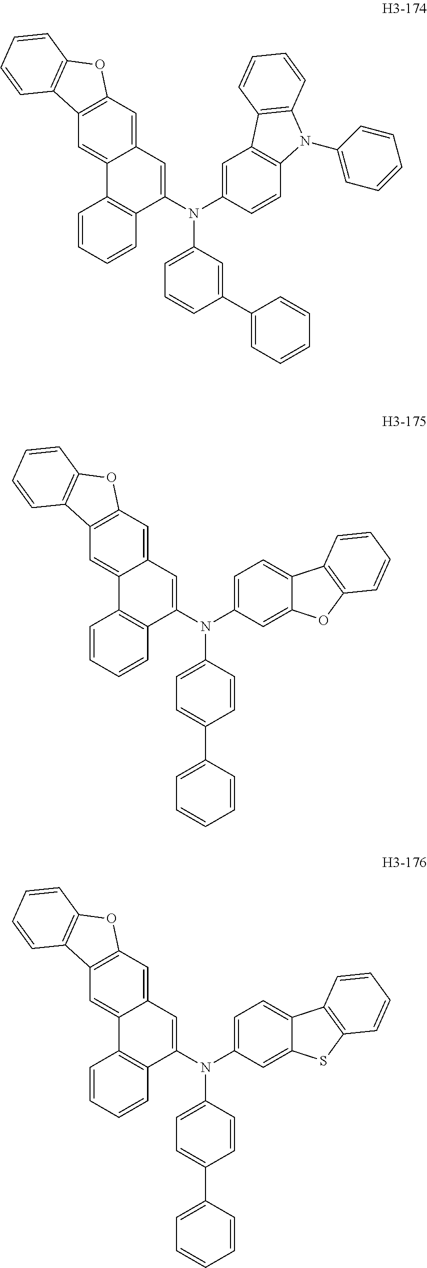 [SMILES: c1ccc(-c2cccc(N(c3ccc4c(c3)c3ccccc3n4-c3ccccc3)c3cc4cc5oc6ccccc6c5cc4c4ccccc34)c2)cc1 ; c1ccc(-c2ccc(N(c3ccc4c(c3)oc3ccccc34)c3cc4cc5oc6ccccc6c5cc4c4ccccc34)cc2)cc1 ; c1ccc(-c2ccc(N(c3ccc4c(c3)sc3ccccc34)c3cc4cc5oc6ccccc6c5cc4c4ccccc34)cc2)cc1]

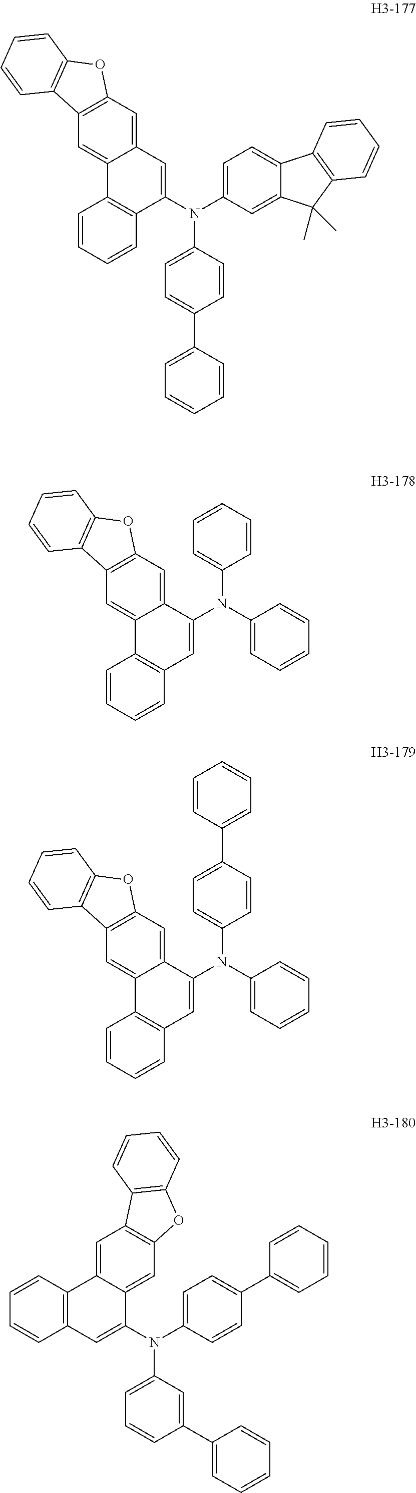 [SMILES: CC1(C)c2ccccc2-c2ccc(N(c3ccc(-c4ccccc4)cc3)c3cc4cc5oc6ccccc6c5cc4c4ccccc34)cc21 ; c1ccc(N(c2ccccc2)c2cc3ccccc3c3cc4c(cc23)oc2ccccc24)cc1 ; c1ccc(-c2ccc(N(c3ccccc3)c3cc4ccccc4c4cc5c(cc34)oc3ccccc35)cc2)cc1 ; c1ccc(-c2ccc(N(c3cccc(-c4ccccc4)c3)c3cc4ccccc4c4cc5c(cc34)oc3ccccc35)cc2)cc1]

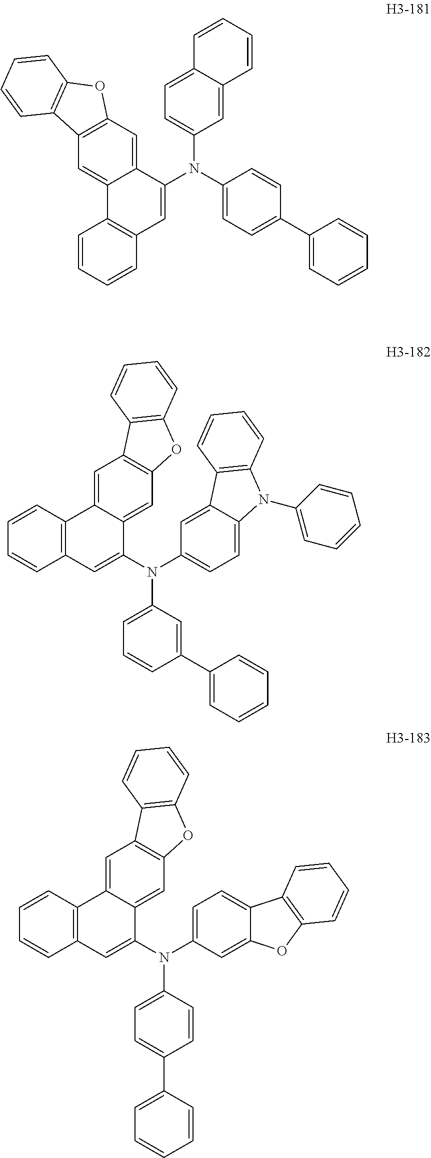 [SMILES: c1ccc(-c2ccc(N(c3ccc4ccccc4c3)c3cc4ccccc4c4cc5c(cc34)oc3ccccc35)cc2)cc1 ; c1ccc(-c2cccc(N(c3ccc4c(c3)c3ccccc3n4-c3ccccc3)c3cc4ccccc4c4cc5c(cc34)oc3ccccc35)c2)cc1 ; c1ccc(-c2ccc(N(c3ccc4c(c3)oc3ccccc34)c3cc4ccccc4c4cc5c(cc34)oc3ccccc35)cc2)cc1]

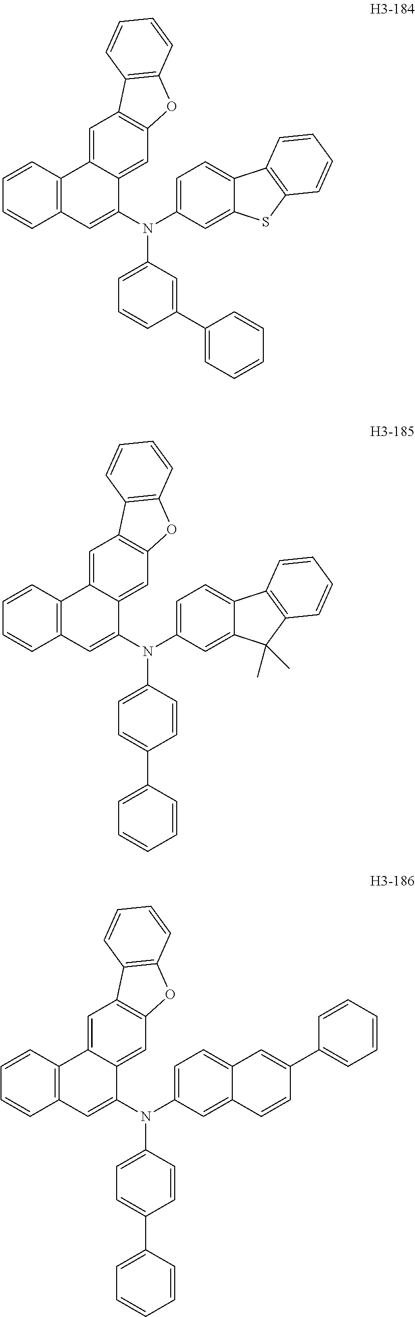 [SMILES: c1ccc(-c2cccc(N(c3ccc4c(c3)sc3ccccc34)c3cc4ccccc4c4cc5c(cc34)oc3ccccc35)c2)cc1 ; CC1(C)c2ccccc2-c2ccc(N(c3ccc(-c4ccccc4)cc3)c3cc4ccccc4c4cc5c(cc34)oc3ccccc35)cc21 ; c1ccc(-c2ccc(N(c3ccc4cc(-c5ccccc5)ccc4c3)c3cc4ccccc4c4cc5c(cc34)oc3ccccc35)cc2)cc1]

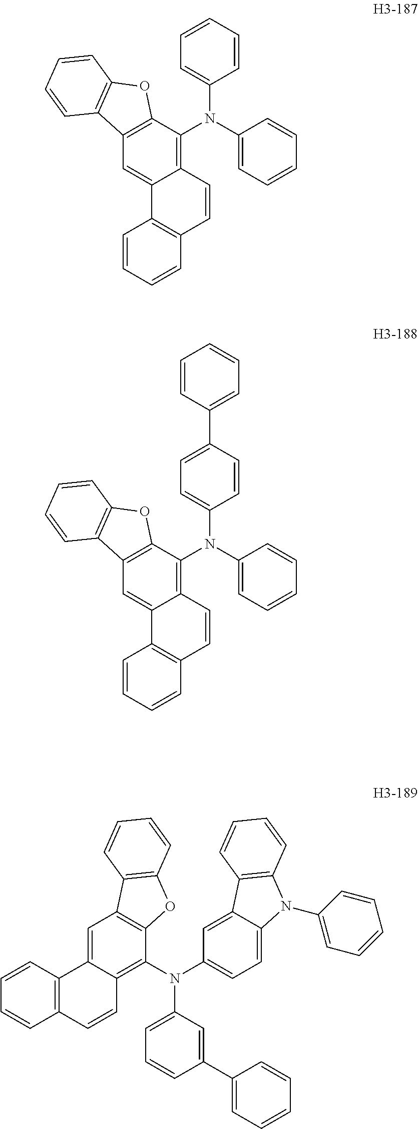 [SMILES: c1ccc(N(c2ccccc2)c2c3ccc4ccccc4c3cc3c2oc2ccccc23)cc1 ; c1ccc(-c2ccc(N(c3ccccc3)c3c4ccc5ccccc5c4cc4c3oc3ccccc34)cc2)cc1 ; c1ccc(-c2cccc(N(c3ccc4c(c3)c3ccccc3n4-c3ccccc3)c3c4ccc5ccccc5c4cc4c3oc3ccccc34)c2)cc1]

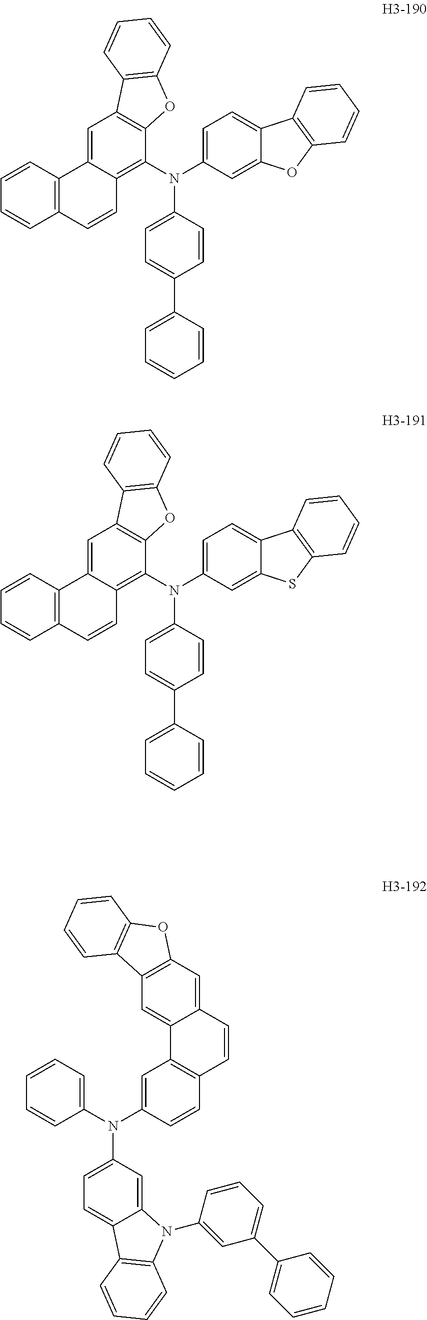 [SMILES: c1ccc(-c2ccc(N(c3ccc4c(c3)oc3ccccc34)c3c4ccc5ccccc5c4cc4c3oc3ccccc34)cc2)cc1 ; c1ccc(-c2ccc(N(c3ccc4c(c3)sc3ccccc34)c3c4ccc5ccccc5c4cc4c3oc3ccccc34)cc2)cc1 ; c1ccc(-c2cccc(-n3c4ccccc4c4ccc(N(c5ccccc5)c5ccc6ccc7cc8oc9ccccc9c8cc7c6c5)cc43)c2)cc1]

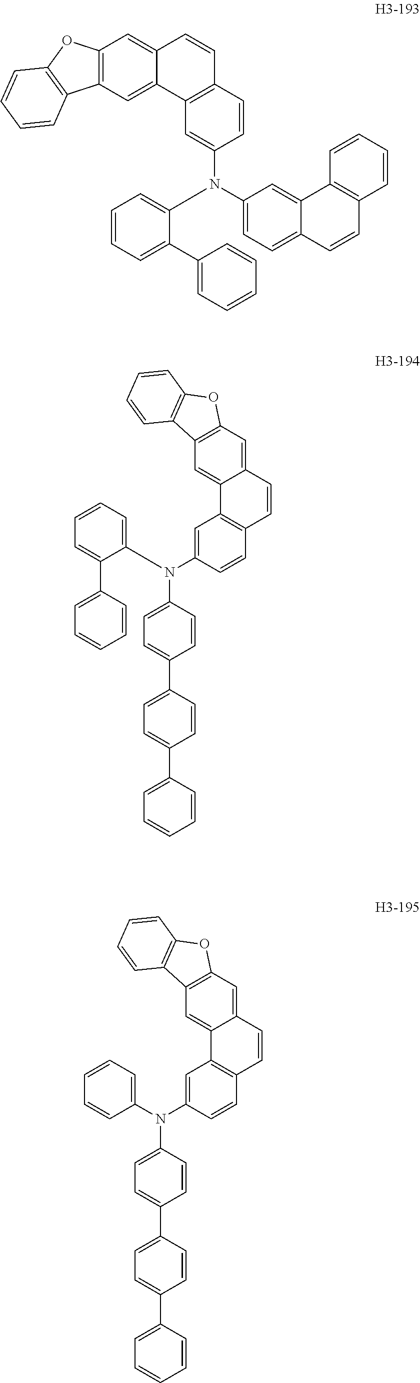 [SMILES: c1ccc(-c2ccccc2N(c2ccc3ccc4ccccc4c3c2)c2ccc3ccc4cc5oc6ccccc6c5cc4c3c2)cc1 ; c1ccc(-c2ccc(-c3ccc(N(c4ccc5ccc6cc7oc8ccccc8c7cc6c5c4)c4ccccc4-c4ccccc4)cc3)cc2)cc1 ; c1ccc(-c2ccc(-c3ccc(N(c4ccccc4)c4ccc5ccc6cc7oc8ccccc8c7cc6c5c4)cc3)cc2)cc1]

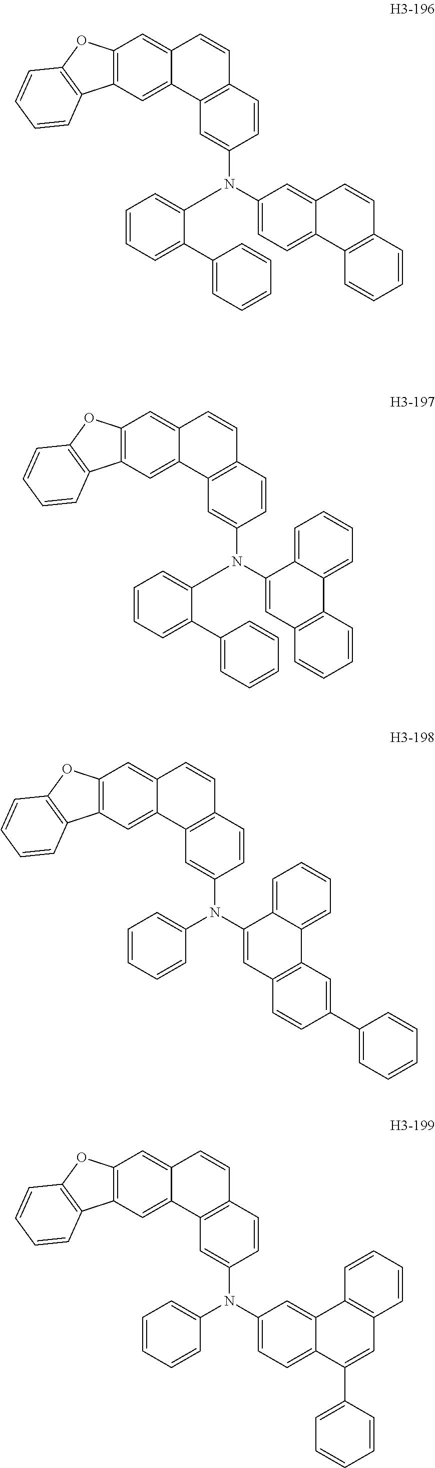 [SMILES: c1ccc(-c2ccccc2N(c2ccc3c(ccc4ccccc43)c2)c2ccc3ccc4cc5oc6ccccc6c5cc4c3c2)cc1 ; c1ccc(-c2ccccc2N(c2ccc3ccc4cc5oc6ccccc6c5cc4c3c2)c2cc3ccccc3c3ccccc23)cc1 ; c1ccc(-c2ccc3cc(N(c4ccccc4)c4ccc5ccc6cc7oc8ccccc8c7cc6c5c4)c4ccccc4c3c2)cc1 ; c1ccc(-c2cc3ccccc3c3cc(N(c4ccccc4)c4ccc5ccc6cc7oc8ccccc8c7cc6c5c4)ccc23)cc1]

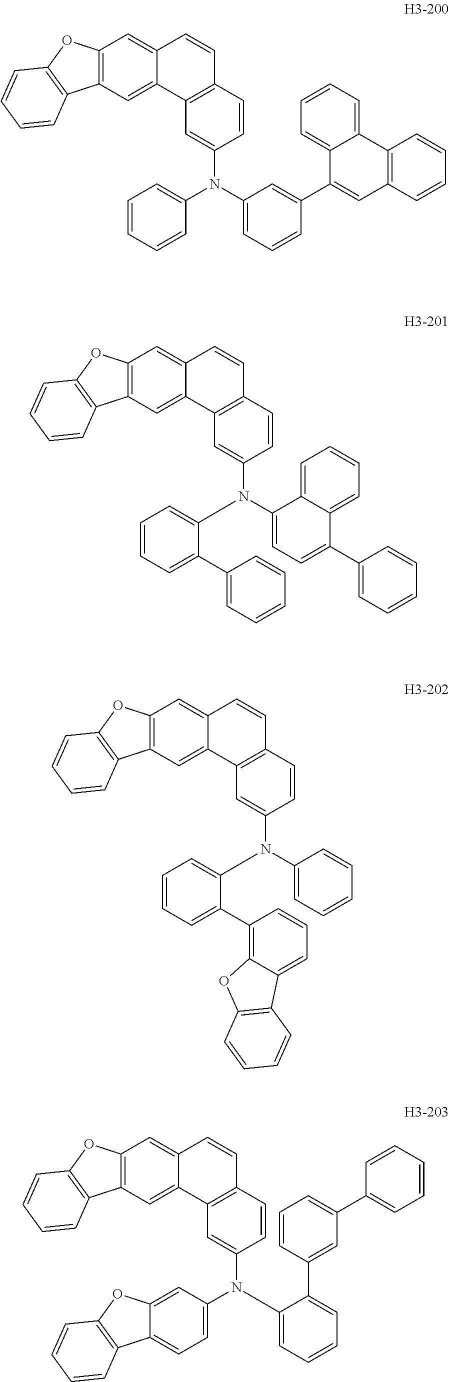 [SMILES: c1ccc(N(c2cccc(-c3cc4ccccc4c4ccccc34)c2)c2ccc3ccc4cc5oc6ccccc6c5cc4c3c2)cc1 ; c1ccc(-c2ccccc2N(c2ccc3ccc4cc5oc6ccccc6c5cc4c3c2)c2ccc(-c3ccccc3)c3ccccc23)cc1 ; c1ccc(N(c2ccc3ccc4cc5oc6ccccc6c5cc4c3c2)c2ccccc2-c2cccc3c2oc2ccccc23)cc1 ; c1ccc(-c2cccc(-c3ccccc3N(c3ccc4c(c3)oc3ccccc34)c3ccc4ccc5cc6oc7ccccc7c6cc5c4c3)c2)cc1]

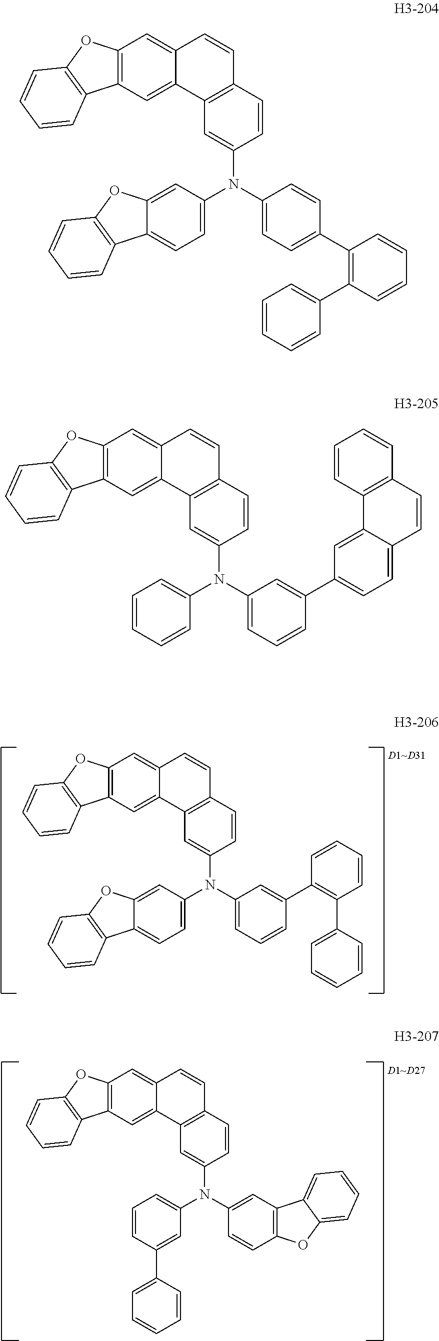

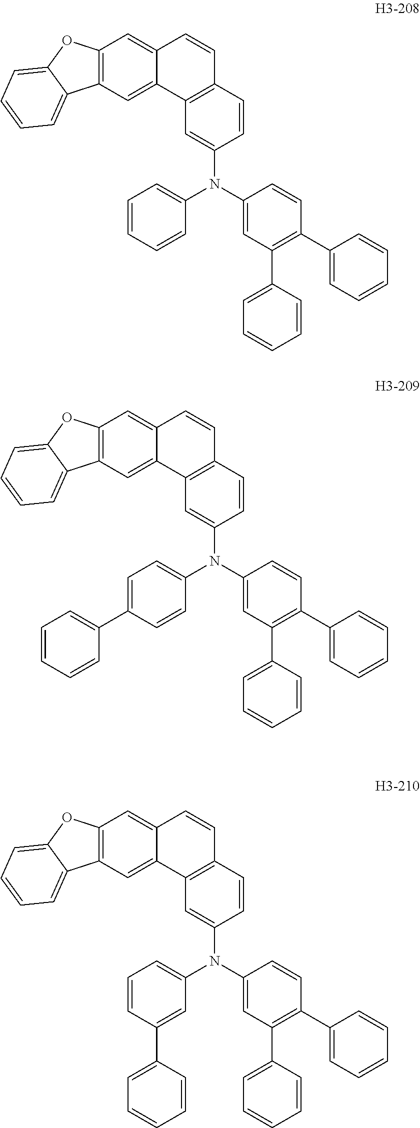 [SMILES: c1ccc(-c2ccc(N(c3ccccc3)c3ccc4ccc5cc6oc7ccccc7c6cc5c4c3)cc2-c2ccccc2)cc1 ; c1ccc(-c2ccc(N(c3ccc(-c4ccccc4)c(-c4ccccc4)c3)c3ccc4ccc5cc6oc7ccccc7c6cc5c4c3)cc2)cc1 ; c1ccc(-c2cccc(N(c3ccc(-c4ccccc4)c(-c4ccccc4)c3)c3ccc4ccc5cc6oc7ccccc7c6cc5c4c3)c2)cc1]

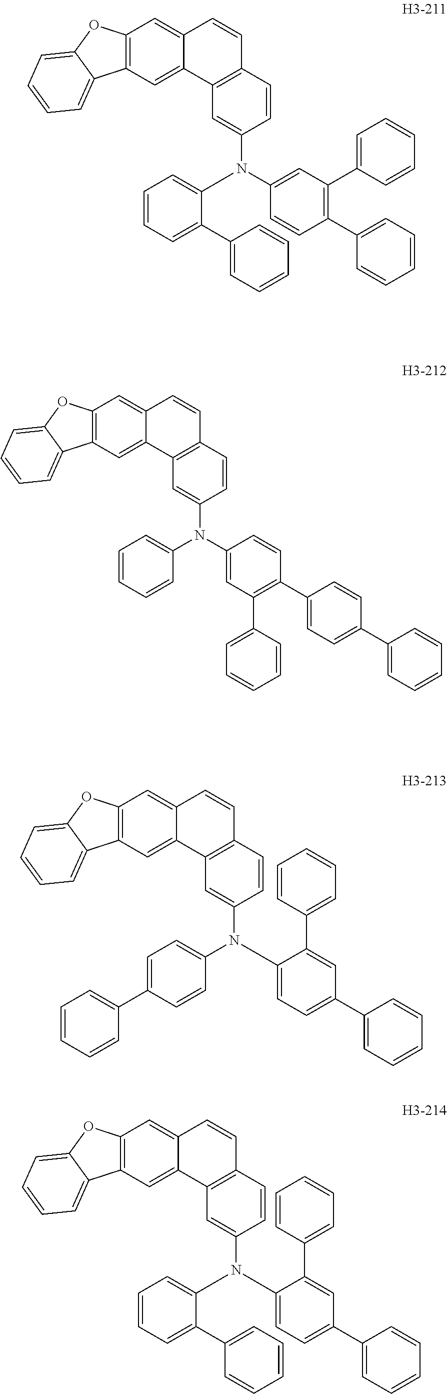 [SMILES: c1ccc(-c2ccc(N(c3ccc4ccc5cc6oc7ccccc7c6cc5c4c3)c3ccccc3-c3ccccc3)cc2-c2ccccc2)cc1 ; c1ccc(-c2ccc(-c3ccc(N(c4ccccc4)c4ccc5ccc6cc7oc8ccccc8c7cc6c5c4)cc3-c3ccccc3)cc2)cc1 ; c1ccc(-c2ccc(N(c3ccc4ccc5cc6oc7ccccc7c6cc5c4c3)c3ccc(-c4ccccc4)cc3-c3ccccc3)cc2)cc1 ; c1ccc(-c2ccc(N(c3ccc4ccc5cc6oc7ccccc7c6cc5c4c3)c3ccccc3-c3ccccc3)c(-c3ccccc3)c2)cc1]

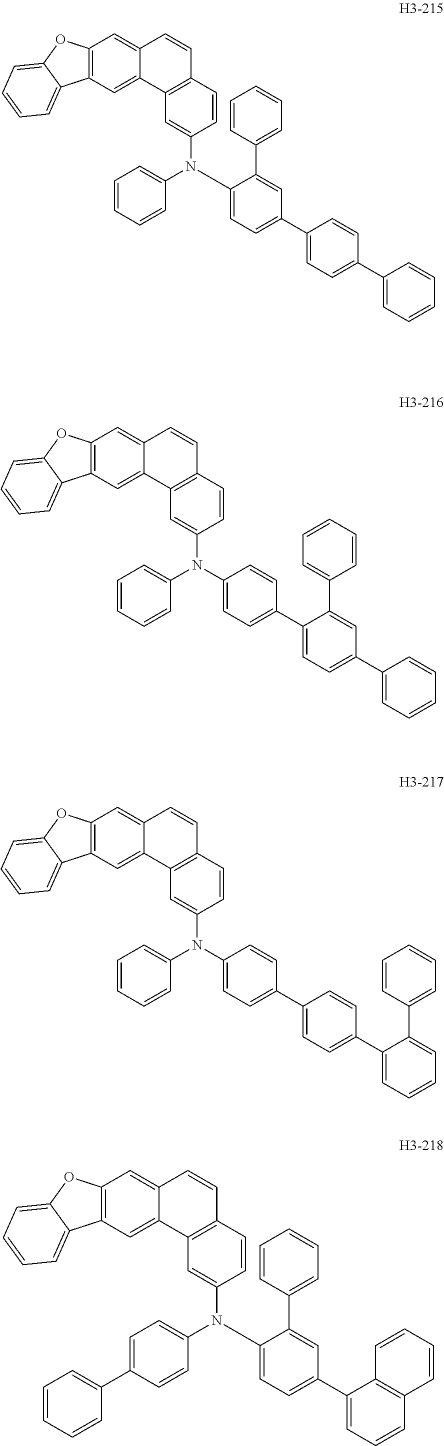 [SMILES: c1ccc(-c2ccc(-c3ccc(N(c4ccccc4)c4ccc5ccc6cc7oc8ccccc8c7cc6c5c4)c(-c4ccccc4)c3)cc2)cc1 ; c1ccc(-c2ccc(-c3ccc(N(c4ccccc4)c4ccc5ccc6cc7oc8ccccc8c7cc6c5c4)cc3)c(-c3ccccc3)c2)cc1 ; c1ccc(-c2ccccc2-c2ccc(-c3ccc(N(c4ccccc4)c4ccc5ccc6cc7oc8ccccc8c7cc6c5c4)cc3)cc2)cc1 ; c1ccc(-c2ccc(N(c3ccc4ccc5cc6oc7ccccc7c6cc5c4c3)c3ccc(-c4cccc5ccccc45)cc3-c3ccccc3)cc2)cc1]

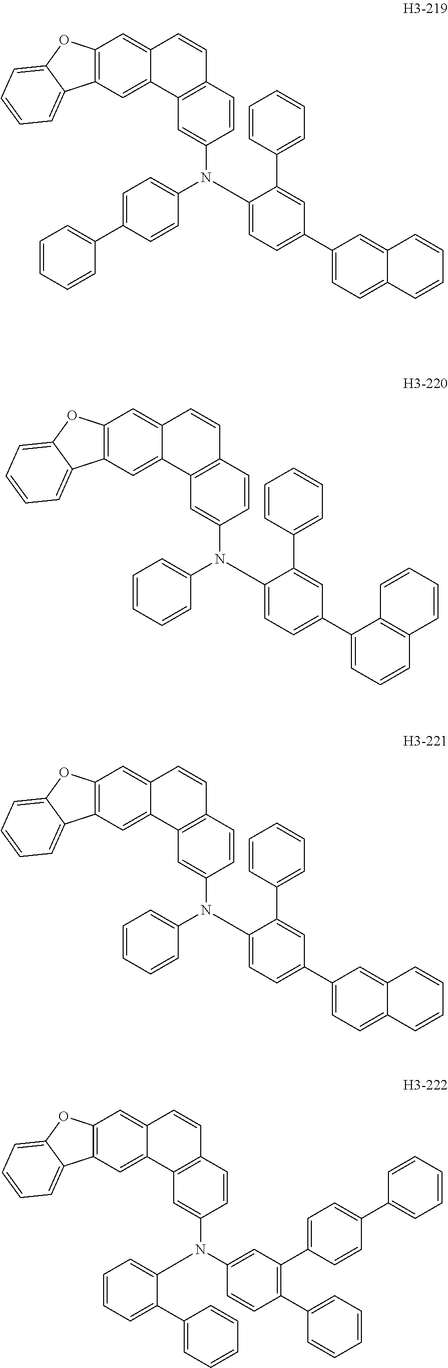 [SMILES: c1ccc(-c2ccc(N(c3ccc4ccc5cc6oc7ccccc7c6cc5c4c3)c3ccc(-c4ccc5ccccc5c4)cc3-c3ccccc3)cc2)cc1 ; c1ccc(-c2cc(-c3cccc4ccccc34)ccc2N(c2ccccc2)c2ccc3ccc4cc5oc6ccccc6c5cc4c3c2)cc1 ; c1ccc(-c2cc(-c3ccc4ccccc4c3)ccc2N(c2ccccc2)c2ccc3ccc4cc5oc6ccccc6c5cc4c3c2)cc1 ; c1ccc(-c2ccc(-c3cc(N(c4ccc5ccc6cc7oc8ccccc8c7cc6c5c4)c4ccccc4-c4ccccc4)ccc3-c3ccccc3)cc2)cc1]

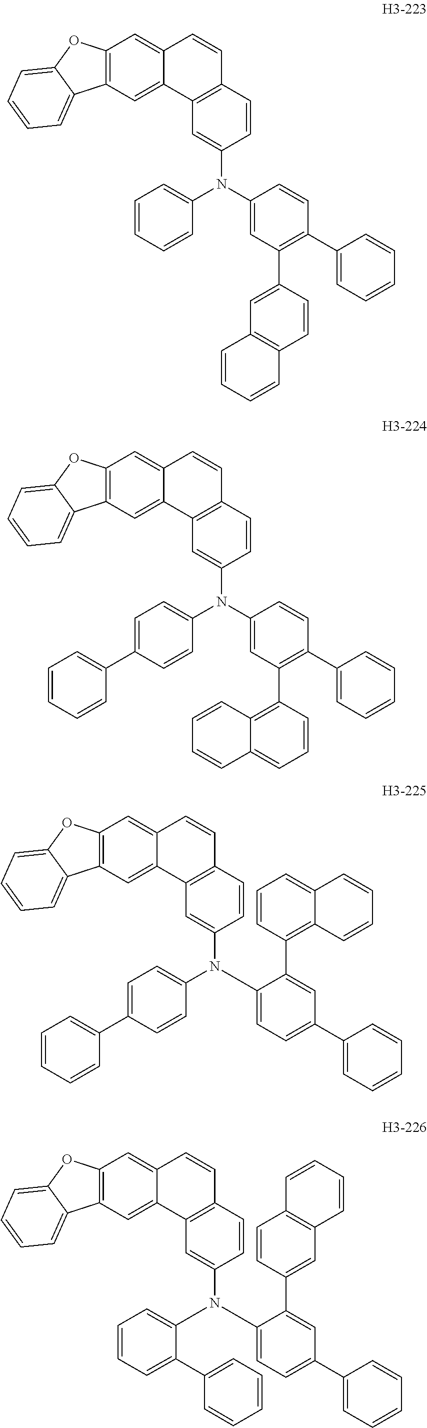 [SMILES: c1ccc(-c2ccc(N(c3ccccc3)c3ccc4ccc5cc6oc7ccccc7c6cc5c4c3)cc2-c2ccc3ccccc3c2)cc1 ; c1ccc(-c2ccc(N(c3ccc(-c4ccccc4)c(-c4cccc5ccccc45)c3)c3ccc4ccc5cc6oc7ccccc7c6cc5c4c3)cc2)cc1 ; c1ccc(-c2ccc(N(c3ccc4ccc5cc6oc7ccccc7c6cc5c4c3)c3ccc(-c4ccccc4)cc3-c3cccc4ccccc34)cc2)cc1 ; c1ccc(-c2ccc(N(c3ccc4ccc5cc6oc7ccccc7c6cc5c4c3)c3ccccc3-c3ccccc3)c(-c3ccc4ccccc4c3)c2)cc1]

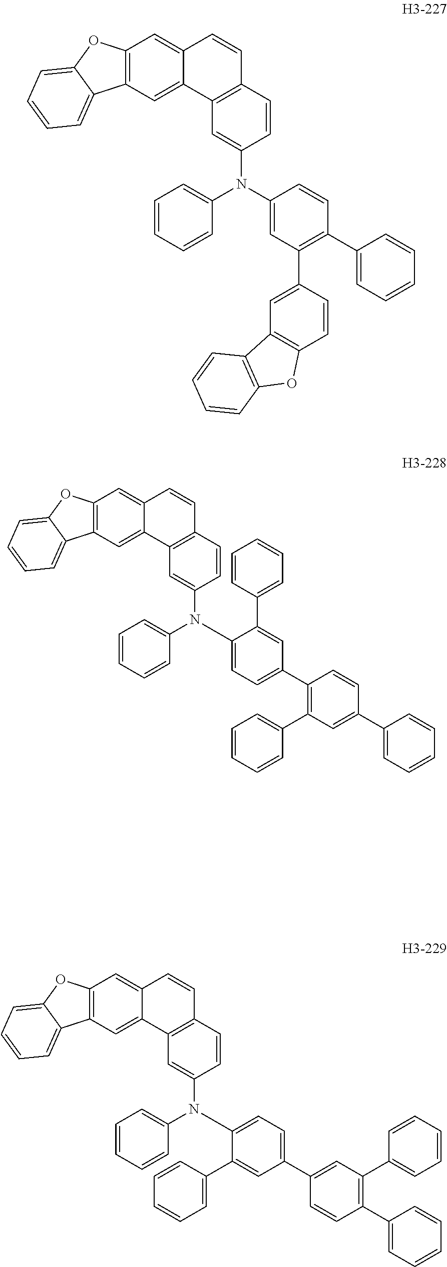 [SMILES: c1ccc(-c2ccc(N(c3ccccc3)c3ccc4ccc5cc6oc7ccccc7c6cc5c4c3)cc2-c2ccc3oc4ccccc4c3c2)cc1 ; c1ccc(-c2ccc(-c3ccc(N(c4ccccc4)c4ccc5ccc6cc7oc8ccccc8c7cc6c5c4)c(-c4ccccc4)c3)c(-c3ccccc3)c2)cc1 ; c1ccc(-c2ccc(-c3ccc(N(c4ccccc4)c4ccc5ccc6cc7oc8ccccc8c7cc6c5c4)c(-c4ccccc4)c3)cc2-c2ccccc2)cc1]

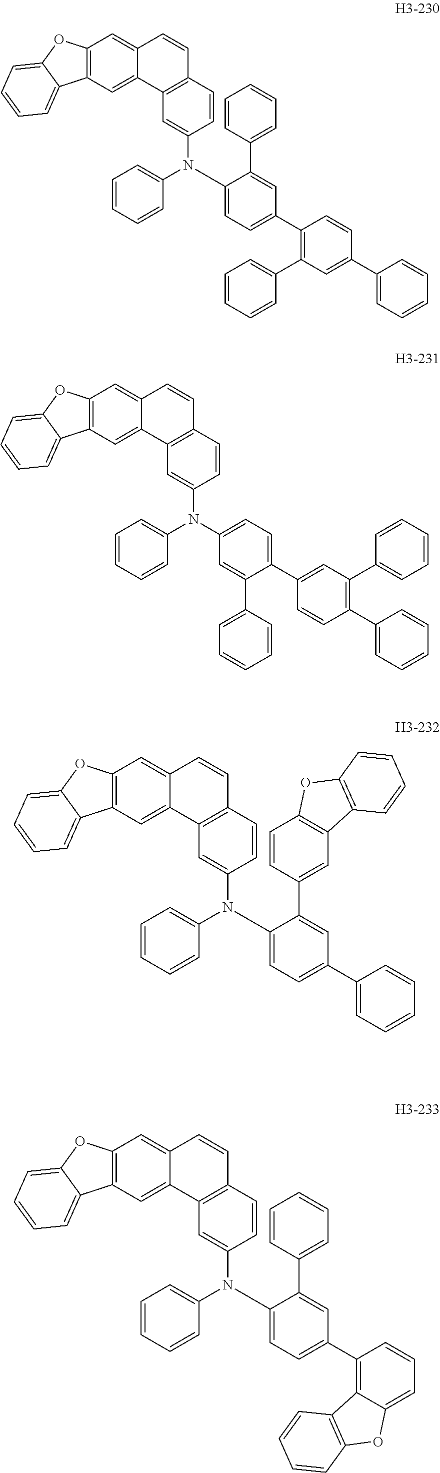 [SMILES: c1ccc(-c2ccc(-c3ccc(N(c4ccccc4)c4ccc5ccc6cc7oc8ccccc8c7cc6c5c4)c(-c4ccccc4)c3)c(-c3ccccc3)c2)cc1 ; c1ccc(-c2ccc(-c3ccc(N(c4ccccc4)c4ccc5ccc6cc7oc8ccccc8c7cc6c5c4)cc3-c3ccccc3)cc2-c2ccccc2)cc1 ; c1ccc(-c2ccc(N(c3ccccc3)c3ccc4ccc5cc6oc7ccccc7c6cc5c4c3)c(-c3ccc4oc5ccccc5c4c3)c2)cc1 ; c1ccc(-c2cc(-c3cccc4oc5ccccc5c34)ccc2N(c2ccccc2)c2ccc3ccc4cc5oc6ccccc6c5cc4c3c2)cc1]

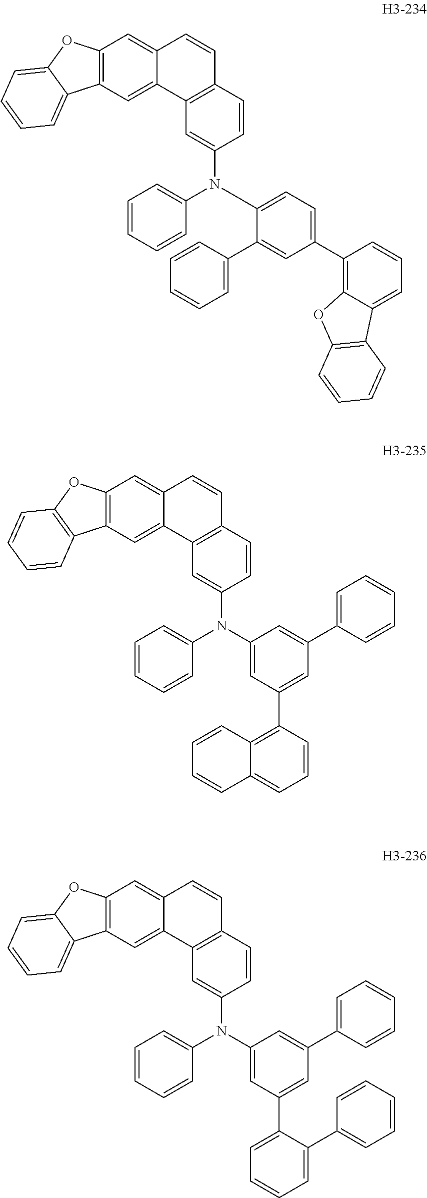 [SMILES: c1ccc(-c2cc(-c3cccc4c3oc3ccccc34)ccc2N(c2ccccc2)c2ccc3ccc4cc5oc6ccccc6c5cc4c3c2)cc1 ; c1ccc(-c2cc(-c3cccc4ccccc34)cc(N(c3ccccc3)c3ccc4ccc5cc6oc7ccccc7c6cc5c4c3)c2)cc1 ; c1ccc(-c2cc(-c3ccccc3-c3ccccc3)cc(N(c3ccccc3)c3ccc4ccc5cc6oc7ccccc7c6cc5c4c3)c2)cc1]

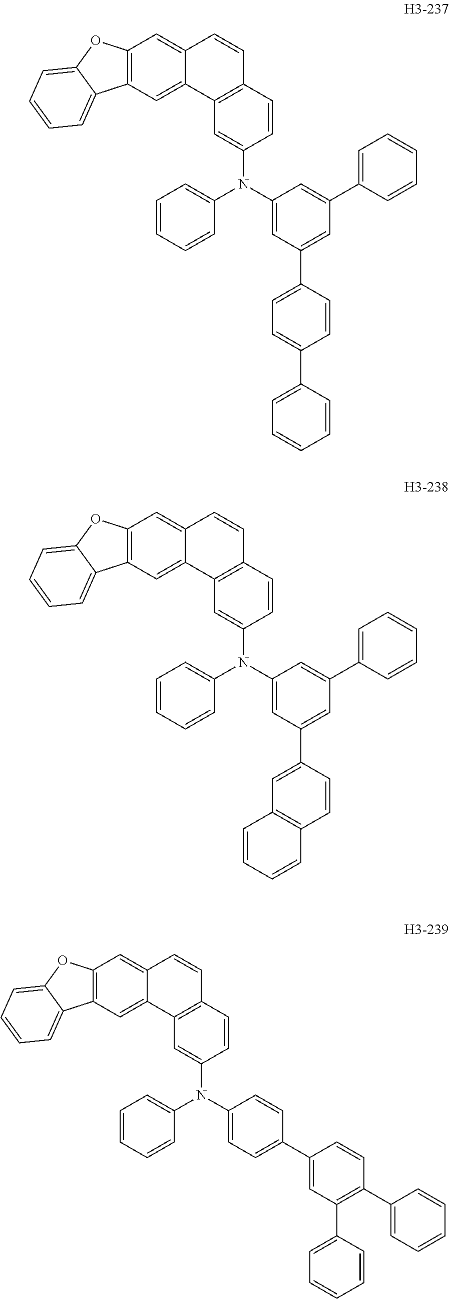 [SMILES: c1ccc(-c2ccc(-c3cc(-c4ccccc4)cc(N(c4ccccc4)c4ccc5ccc6cc7oc8ccccc8c7cc6c5c4)c3)cc2)cc1 ; c1ccc(-c2cc(-c3ccc4ccccc4c3)cc(N(c3ccccc3)c3ccc4ccc5cc6oc7ccccc7c6cc5c4c3)c2)cc1 ; c1ccc(-c2ccc(-c3ccc(N(c4ccccc4)c4ccc5ccc6cc7oc8ccccc8c7cc6c5c4)cc3)cc2-c2ccccc2)cc1]

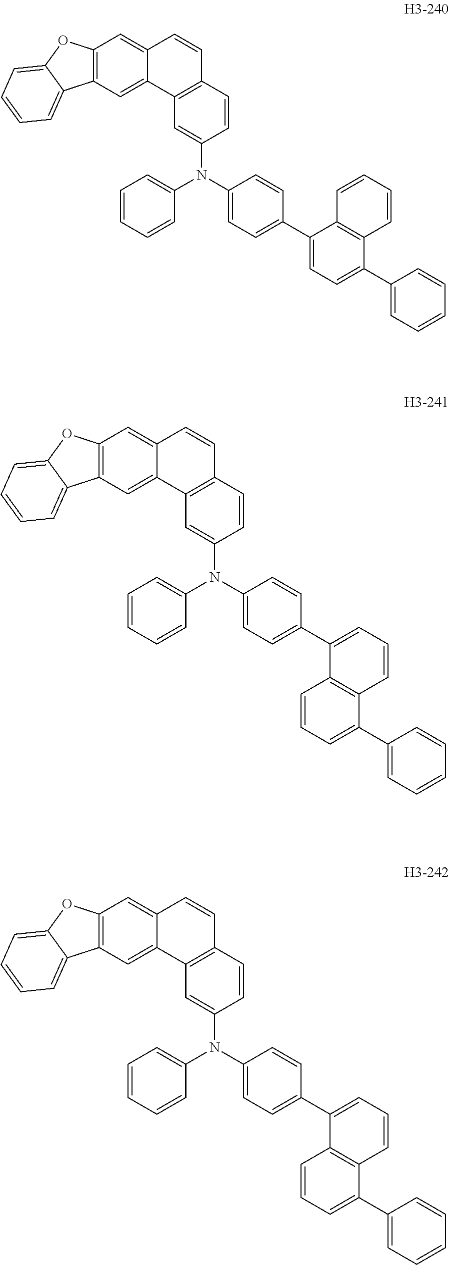 [SMILES: c1ccc(-c2ccc(-c3ccc(N(c4ccccc4)c4ccc5ccc6cc7oc8ccccc8c7cc6c5c4)cc3)c3ccccc23)cc1 ; c1ccc(-c2cccc3c(-c4ccc(N(c5ccccc5)c5ccc6ccc7cc8oc9ccccc9c8cc7c6c5)cc4)cccc23)cc1 ; c1ccc(-c2cccc3c(-c4ccc(N(c5ccccc5)c5ccc6ccc7cc8oc9ccccc9c8cc7c6c5)cc4)cccc23)cc1]

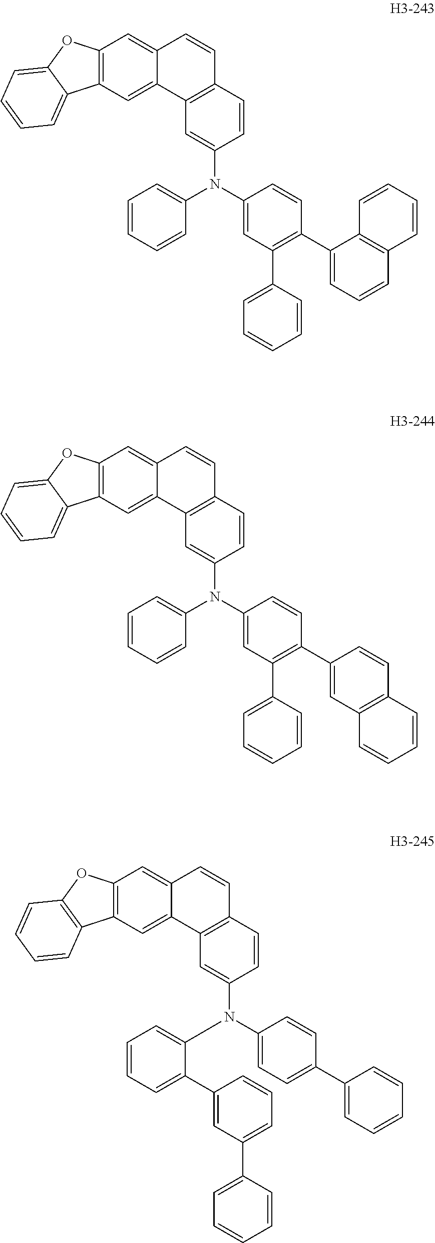 [SMILES: c1ccc(-c2cc(N(c3ccccc3)c3ccc4ccc5cc6oc7ccccc7c6cc5c4c3)ccc2-c2cccc3ccccc23)cc1 ; c1ccc(-c2cc(N(c3ccccc3)c3ccc4ccc5cc6oc7ccccc7c6cc5c4c3)ccc2-c2ccc3ccccc3c2)cc1 ; c1ccc(-c2ccc(N(c3ccc4ccc5cc6oc7ccccc7c6cc5c4c3)c3ccccc3-c3cccc(-c4ccccc4)c3)cc2)cc1]

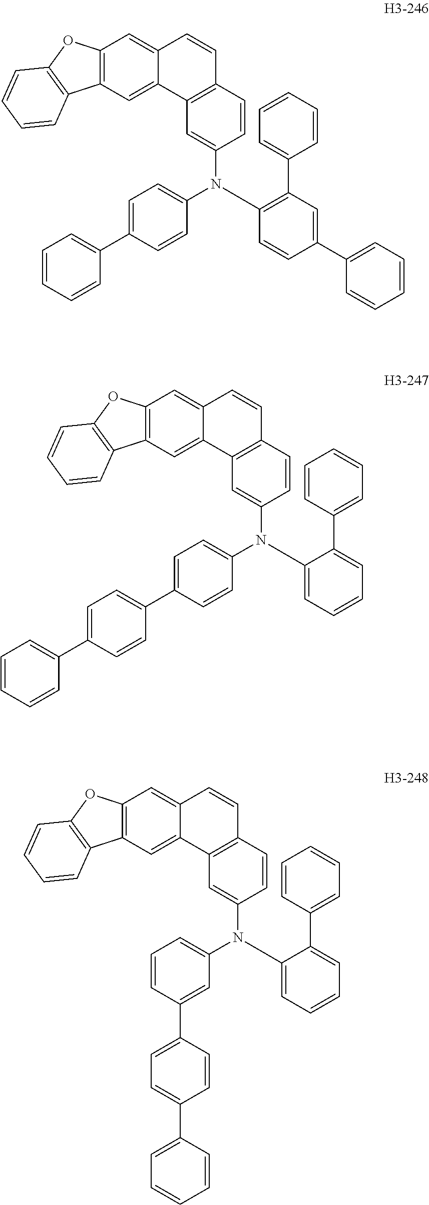 [SMILES: c1ccc(-c2ccc(N(c3ccc4ccc5cc6oc7ccccc7c6cc5c4c3)c3ccc(-c4ccccc4)cc3-c3ccccc3)cc2)cc1 ; c1ccc(-c2ccc(-c3ccc(N(c4ccc5ccc6cc7oc8ccccc8c7cc6c5c4)c4ccccc4-c4ccccc4)cc3)cc2)cc1 ; c1ccc(-c2ccc(-c3cccc(N(c4ccc5ccc6cc7oc8ccccc8c7cc6c5c4)c4ccccc4-c4ccccc4)c3)cc2)cc1]

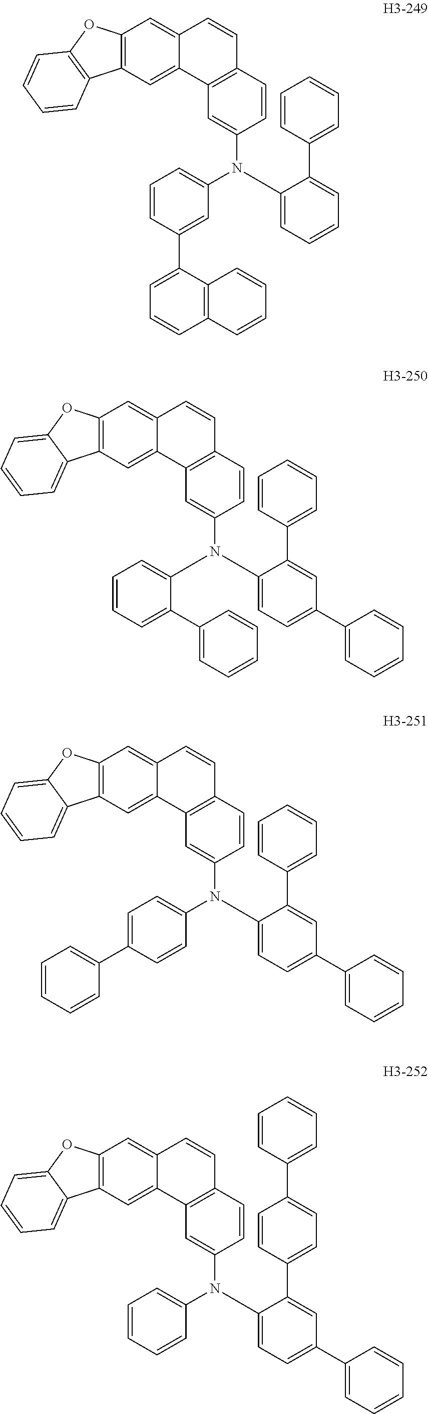 [SMILES: c1ccc(-c2ccccc2N(c2cccc(-c3cccc4ccccc34)c2)c2ccc3ccc4cc5oc6ccccc6c5cc4c3c2)cc1 ; c1ccc(-c2ccc(N(c3ccc4ccc5cc6oc7ccccc7c6cc5c4c3)c3ccccc3-c3ccccc3)c(-c3ccccc3)c2)cc1 ; c1ccc(-c2ccc(N(c3ccc4ccc5cc6oc7ccccc7c6cc5c4c3)c3ccc(-c4ccccc4)cc3-c3ccccc3)cc2)cc1 ; c1ccc(-c2ccc(-c3cc(-c4ccccc4)ccc3N(c3ccccc3)c3ccc4ccc5cc6oc7ccccc7c6cc5c4c3)cc2)cc1]

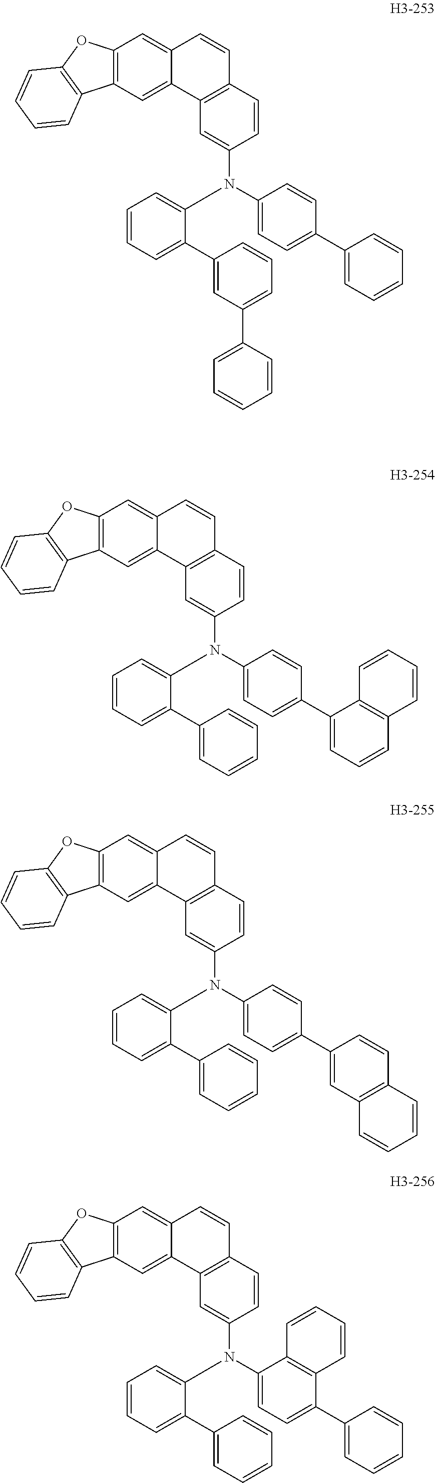 [SMILES: c1ccc(-c2ccc(N(c3ccc4ccc5cc6oc7ccccc7c6cc5c4c3)c3ccccc3-c3cccc(-c4ccccc4)c3)cc2)cc1 ; c1ccc(-c2ccccc2N(c2ccc(-c3cccc4ccccc34)cc2)c2ccc3ccc4cc5oc6ccccc6c5cc4c3c2)cc1 ; c1ccc(-c2ccccc2N(c2ccc(-c3ccc4ccccc4c3)cc2)c2ccc3ccc4cc5oc6ccccc6c5cc4c3c2)cc1 ; c1ccc(-c2ccccc2N(c2ccc3ccc4cc5oc6ccccc6c5cc4c3c2)c2ccc(-c3ccccc3)c3ccccc23)cc1]

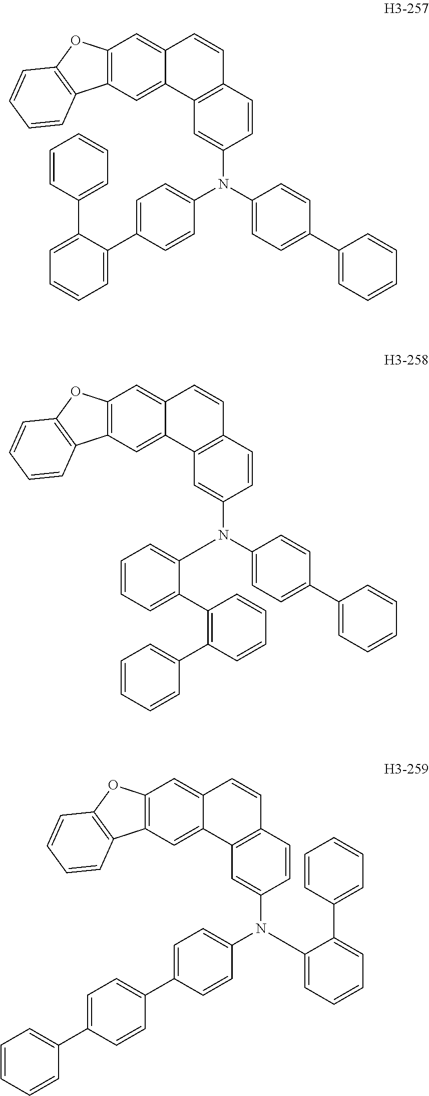 [SMILES: c1ccc(-c2ccc(N(c3ccc(-c4ccccc4-c4ccccc4)cc3)c3ccc4ccc5cc6oc7ccccc7c6cc5c4c3)cc2)cc1 ; c1ccc(-c2ccc(N(c3ccc4ccc5cc6oc7ccccc7c6cc5c4c3)c3ccccc3-c3ccccc3-c3ccccc3)cc2)cc1 ; c1ccc(-c2ccc(-c3ccc(N(c4ccc5ccc6cc7oc8ccccc8c7cc6c5c4)c4ccccc4-c4ccccc4)cc3)cc2)cc1]

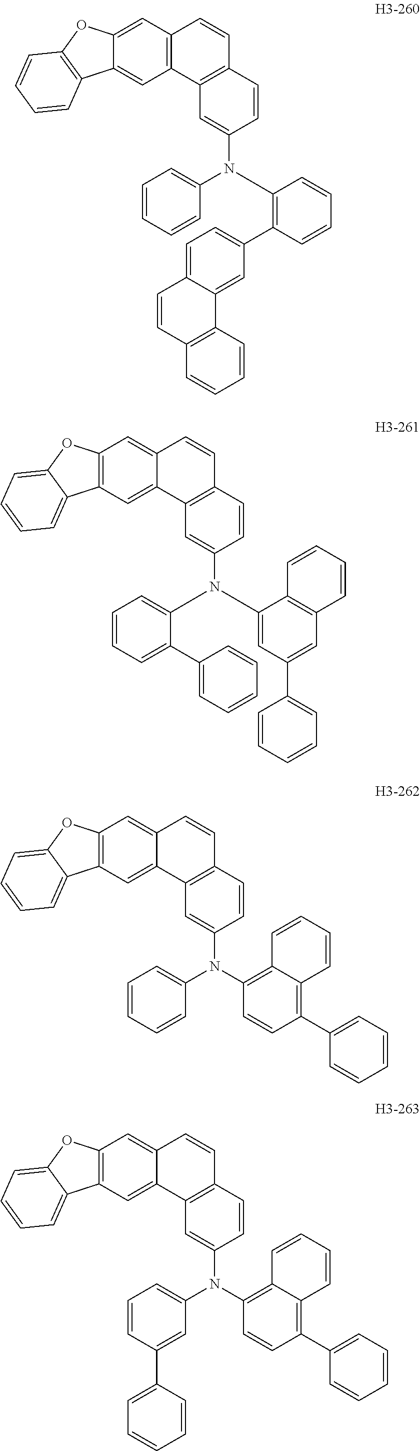 [SMILES: c1ccc(N(c2ccc3ccc4cc5oc6ccccc6c5cc4c3c2)c2ccccc2-c2ccc3ccc4ccccc4c3c2)cc1 ; c1ccc(-c2cc(N(c3ccc4ccc5cc6oc7ccccc7c6cc5c4c3)c3ccccc3-c3ccccc3)c3ccccc3c2)cc1 ; c1ccc(-c2ccc(N(c3ccccc3)c3ccc4ccc5cc6oc7ccccc7c6cc5c4c3)c3ccccc23)cc1 ; c1ccc(-c2cccc(N(c3ccc4ccc5cc6oc7ccccc7c6cc5c4c3)c3ccc(-c4ccccc4)c4ccccc34)c2)cc1]

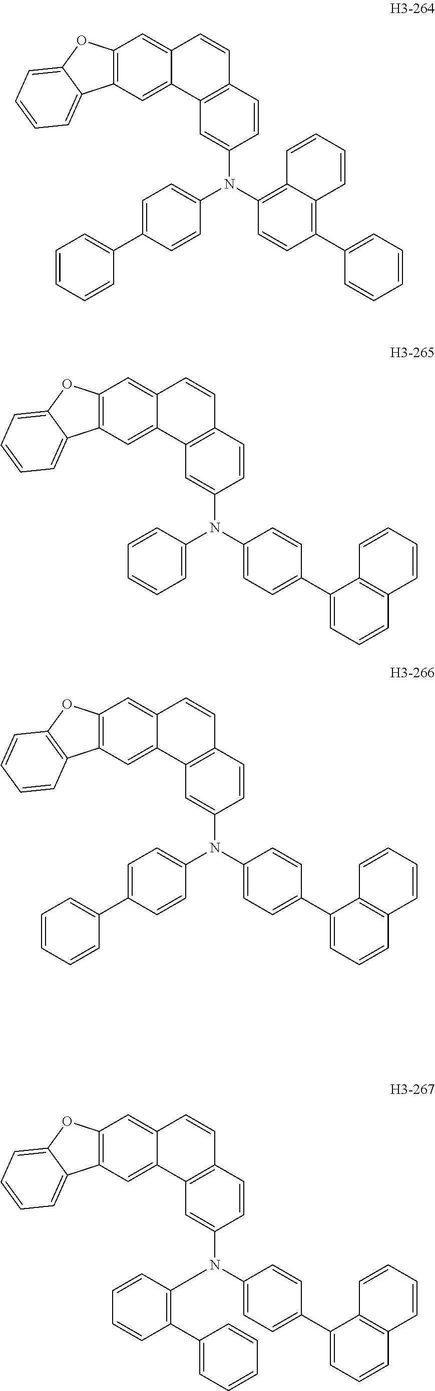 [SMILES: c1ccc(-c2ccc(N(c3ccc4ccc5cc6oc7ccccc7c6cc5c4c3)c3ccc(-c4ccccc4)c4ccccc34)cc2)cc1 ; c1ccc(N(c2ccc(-c3cccc4ccccc34)cc2)c2ccc3ccc4cc5oc6ccccc6c5cc4c3c2)cc1 ; c1ccc(-c2ccc(N(c3ccc(-c4cccc5ccccc45)cc3)c3ccc4ccc5cc6oc7ccccc7c6cc5c4c3)cc2)cc1 ; c1ccc(-c2ccccc2N(c2ccc(-c3cccc4ccccc34)cc2)c2ccc3ccc4cc5oc6ccccc6c5cc4c3c2)cc1]

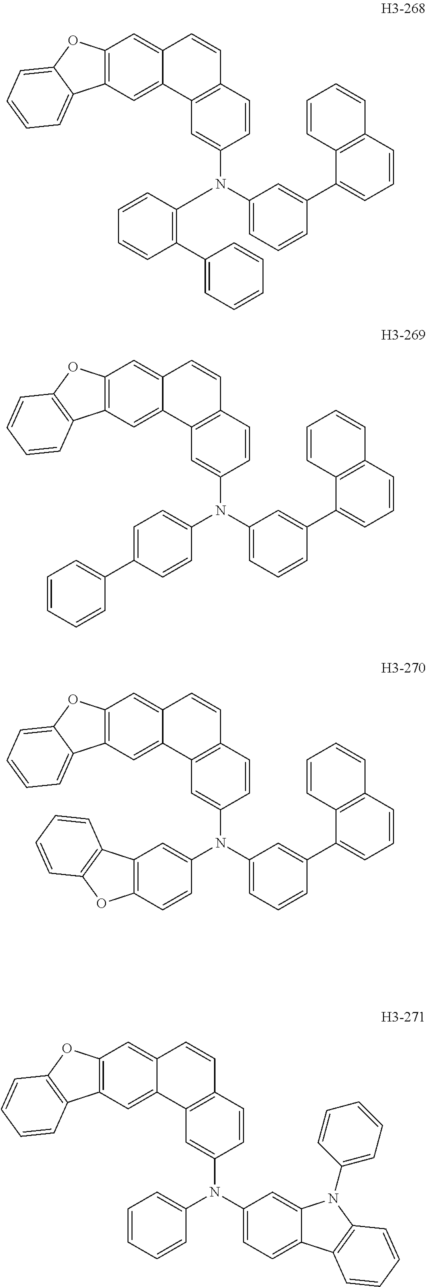 [SMILES: c1ccc(-c2ccccc2N(c2cccc(-c3cccc4ccccc34)c2)c2ccc3ccc4cc5oc6ccccc6c5cc4c3c2)cc1 ; c1ccc(-c2ccc(N(c3cccc(-c4cccc5ccccc45)c3)c3ccc4ccc5cc6oc7ccccc7c6cc5c4c3)cc2)cc1 ; c1cc(-c2cccc3ccccc23)cc(N(c2ccc3ccc4cc5oc6ccccc6c5cc4c3c2)c2ccc3oc4ccccc4c3c2)c1 ; c1ccc(N(c2ccc3ccc4cc5oc6ccccc6c5cc4c3c2)c2ccc3c4ccccc4n(-c4ccccc4)c3c2)cc1]

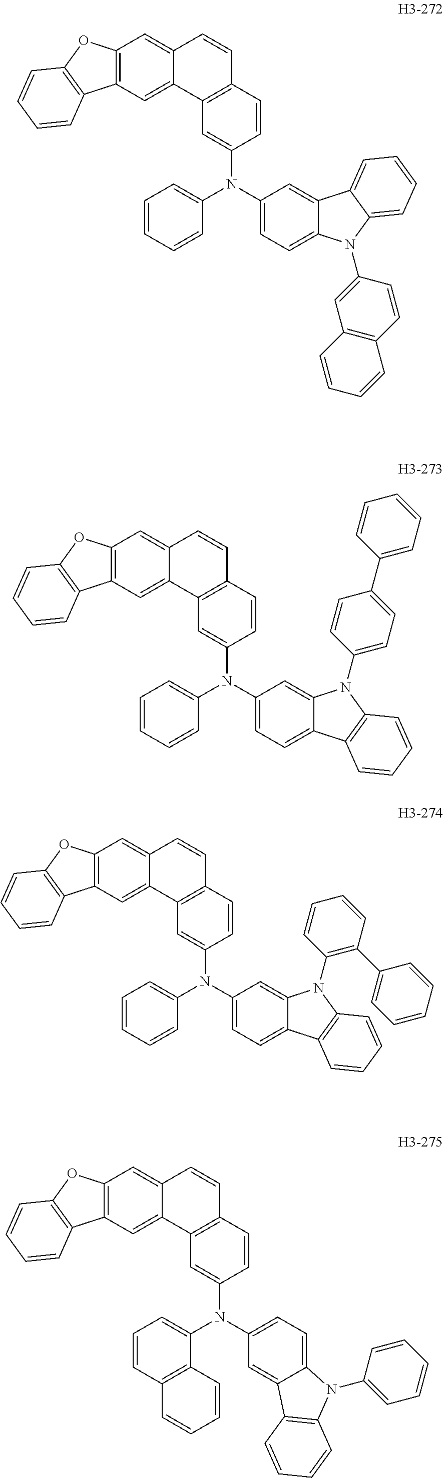 [SMILES: c1ccc(N(c2ccc3ccc4cc5oc6ccccc6c5cc4c3c2)c2ccc3c(c2)c2ccccc2n3-c2ccc3ccccc3c2)cc1 ; c1ccc(-c2ccc(-n3c4ccccc4c4ccc(N(c5ccccc5)c5ccc6ccc7cc8oc9ccccc9c8cc7c6c5)cc43)cc2)cc1 ; c1ccc(-c2ccccc2-n2c3ccccc3c3ccc(N(c4ccccc4)c4ccc5ccc6cc7oc8ccccc8c7cc6c5c4)cc32)cc1 ; c1ccc(-n2c3ccccc3c3cc(N(c4ccc5ccc6cc7oc8ccccc8c7cc6c5c4)c4cccc5ccccc45)ccc32)cc1]

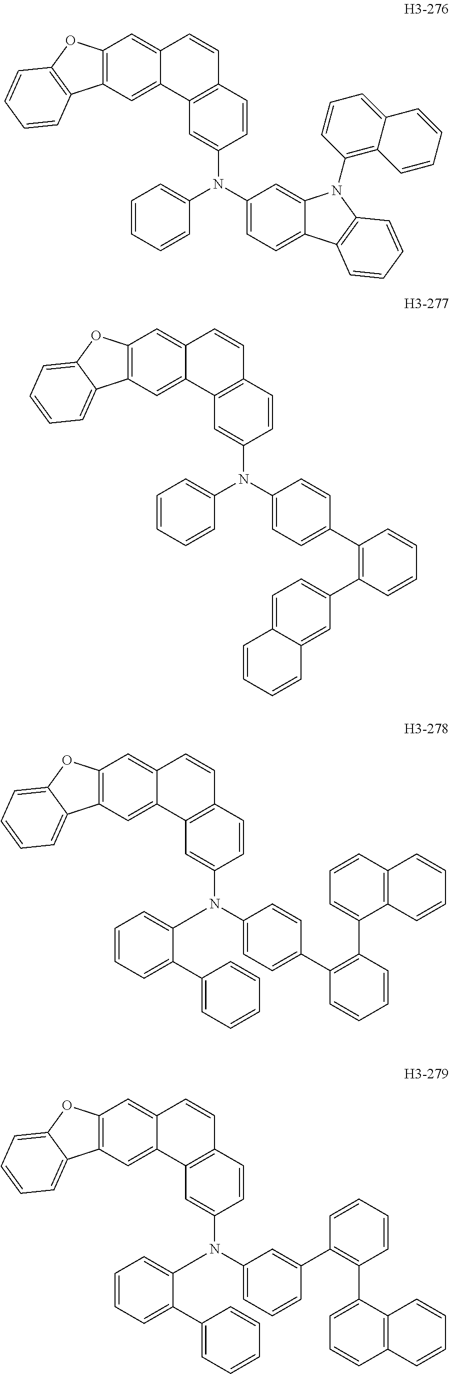 [SMILES: c1ccc(N(c2ccc3ccc4cc5oc6ccccc6c5cc4c3c2)c2ccc3c4ccccc4n(-c4cccc5ccccc45)c3c2)cc1 ; c1ccc(N(c2ccc(-c3ccccc3-c3ccc4ccccc4c3)cc2)c2ccc3ccc4cc5oc6ccccc6c5cc4c3c2)cc1 ; c1ccc(-c2ccccc2N(c2ccc(-c3ccccc3-c3cccc4ccccc34)cc2)c2ccc3ccc4cc5oc6ccccc6c5cc4c3c2)cc1 ; c1ccc(-c2ccccc2N(c2cccc(-c3ccccc3-c3cccc4ccccc34)c2)c2ccc3ccc4cc5oc6ccccc6c5cc4c3c2)cc1]

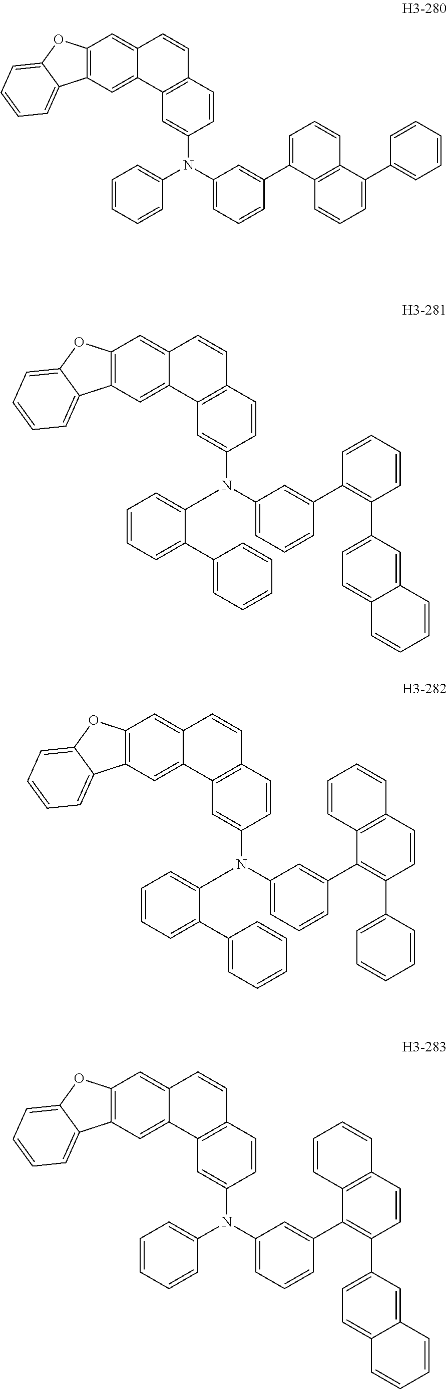 [SMILES: c1ccc(-c2cccc3c(-c4cccc(N(c5ccccc5)c5ccc6ccc7cc8oc9ccccc9c8cc7c6c5)c4)cccc23)cc1 ; c1ccc(-c2ccccc2N(c2cccc(-c3ccccc3-c3ccc4ccccc4c3)c2)c2ccc3ccc4cc5oc6ccccc6c5cc4c3c2)cc1 ; c1ccc(-c2ccccc2N(c2cccc(-c3c(-c4ccccc4)ccc4ccccc34)c2)c2ccc3ccc4cc5oc6ccccc6c5cc4c3c2)cc1 ; c1ccc(N(c2cccc(-c3c(-c4ccc5ccccc5c4)ccc4ccccc34)c2)c2ccc3ccc4cc5oc6ccccc6c5cc4c3c2)cc1]

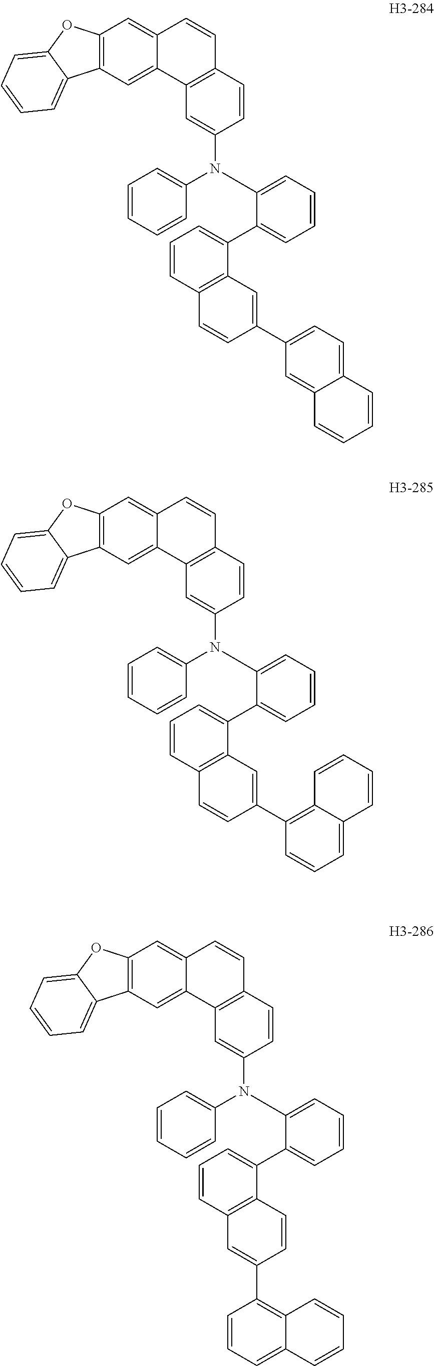 [SMILES: c1ccc(N(c2ccc3ccc4cc5oc6ccccc6c5cc4c3c2)c2ccccc2-c2cccc3ccc(-c4ccc5ccccc5c4)cc23)cc1 ; c1ccc(N(c2ccc3ccc4cc5oc6ccccc6c5cc4c3c2)c2ccccc2-c2cccc3ccc(-c4cccc5ccccc45)cc23)cc1 ; c1ccc(N(c2ccc3ccc4cc5oc6ccccc6c5cc4c3c2)c2ccccc2-c2cccc3cc(-c4cccc5ccccc45)ccc23)cc1]

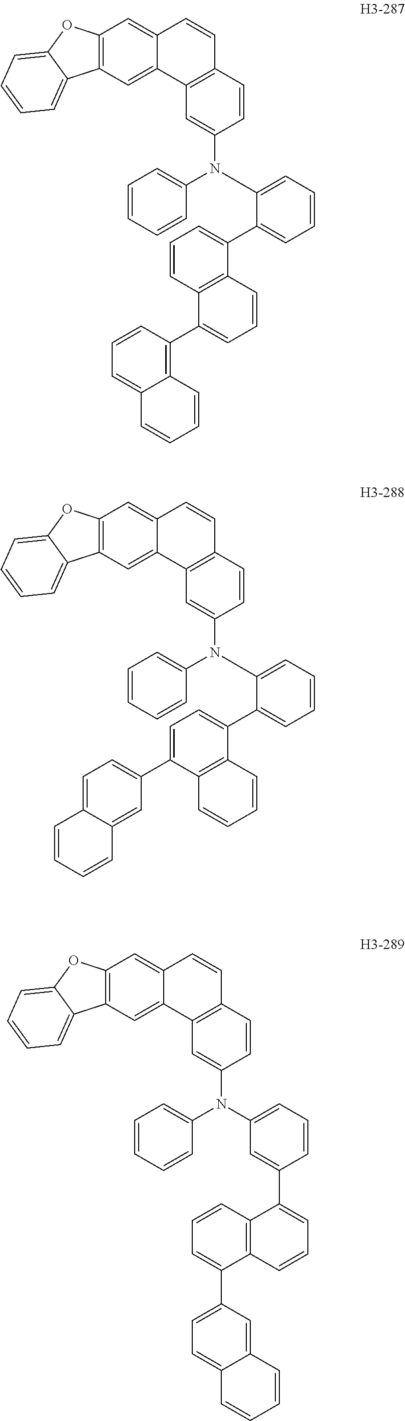 [SMILES: c1ccc(N(c2ccc3ccc4cc5oc6ccccc6c5cc4c3c2)c2ccccc2-c2cccc3c(-c4cccc5ccccc45)cccc23)cc1 ; c1ccc(N(c2ccc3ccc4cc5oc6ccccc6c5cc4c3c2)c2ccccc2-c2ccc(-c3ccc4ccccc4c3)c3ccccc23)cc1 ; c1ccc(N(c2cccc(-c3cccc4c(-c5ccc6ccccc6c5)cccc34)c2)c2ccc3ccc4cc5oc6ccccc6c5cc4c3c2)cc1]

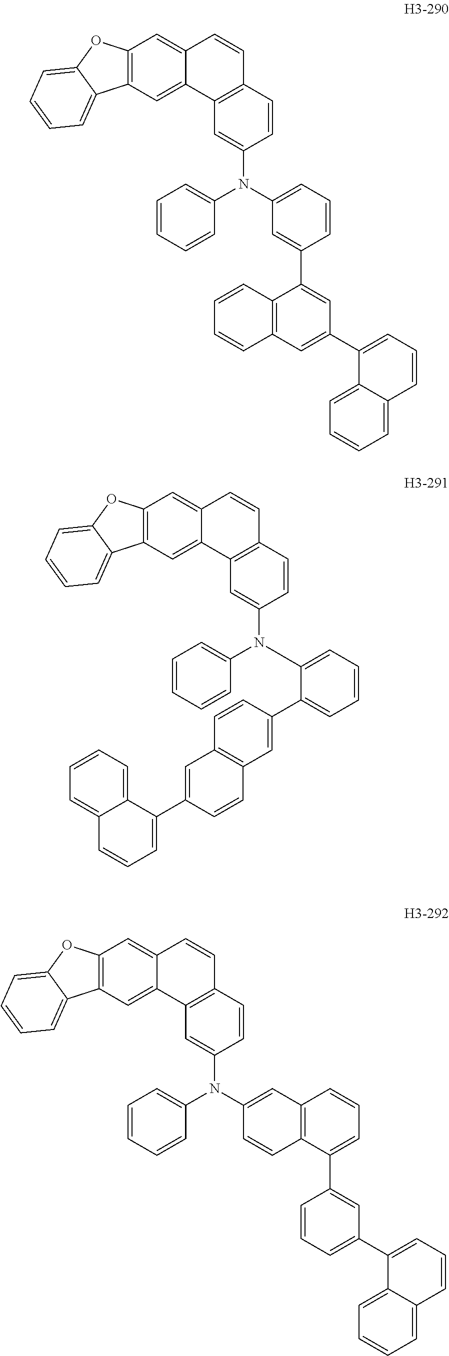 [SMILES: c1ccc(N(c2cccc(-c3cc(-c4cccc5ccccc45)cc4ccccc34)c2)c2ccc3ccc4cc5oc6ccccc6c5cc4c3c2)cc1 ; c1ccc(N(c2ccc3ccc4cc5oc6ccccc6c5cc4c3c2)c2ccccc2-c2ccc3cc(-c4cccc5ccccc45)ccc3c2)cc1 ; c1ccc(N(c2ccc3c(-c4cccc(-c5cccc6ccccc56)c4)cccc3c2)c2ccc3ccc4cc5oc6ccccc6c5cc4c3c2)cc1]

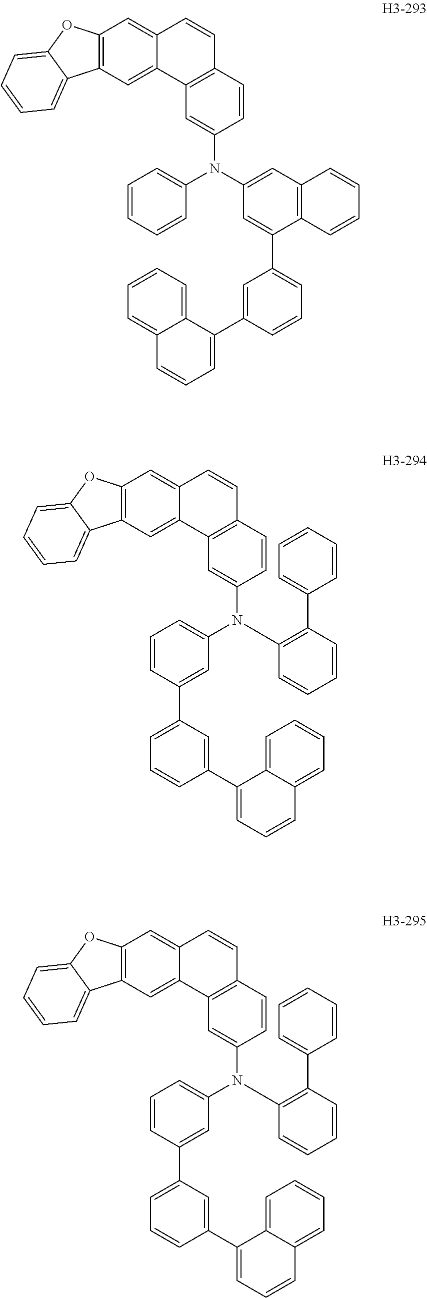 [SMILES: c1ccc(N(c2cc(-c3cccc(-c4cccc5ccccc45)c3)c3ccccc3c2)c2ccc3ccc4cc5oc6ccccc6c5cc4c3c2)cc1 ; c1ccc(-c2ccccc2N(c2cccc(-c3cccc(-c4cccc5ccccc45)c3)c2)c2ccc3ccc4cc5oc6ccccc6c5cc4c3c2)cc1 ; c1ccc(-c2ccccc2N(c2cccc(-c3cccc(-c4cccc5ccccc45)c3)c2)c2ccc3ccc4cc5oc6ccccc6c5cc4c3c2)cc1]

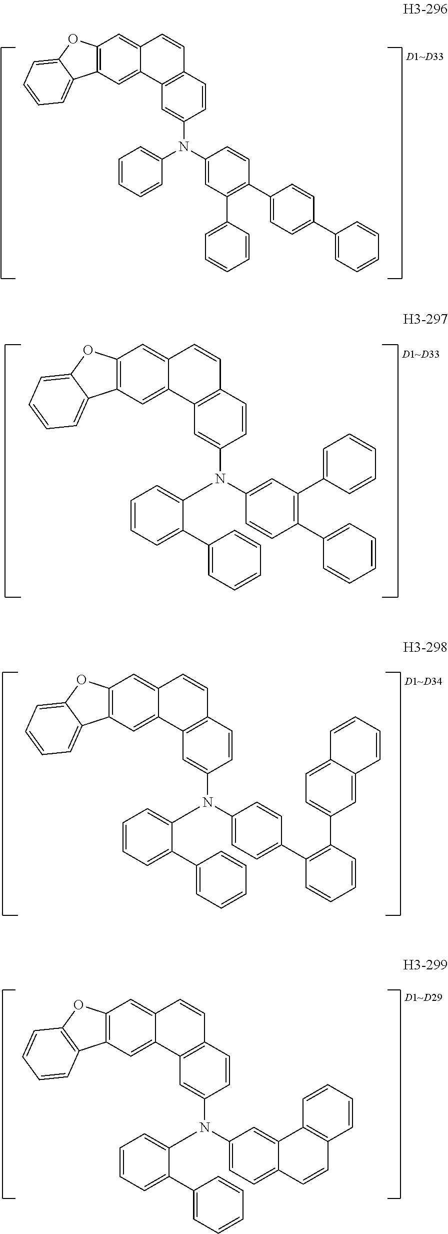

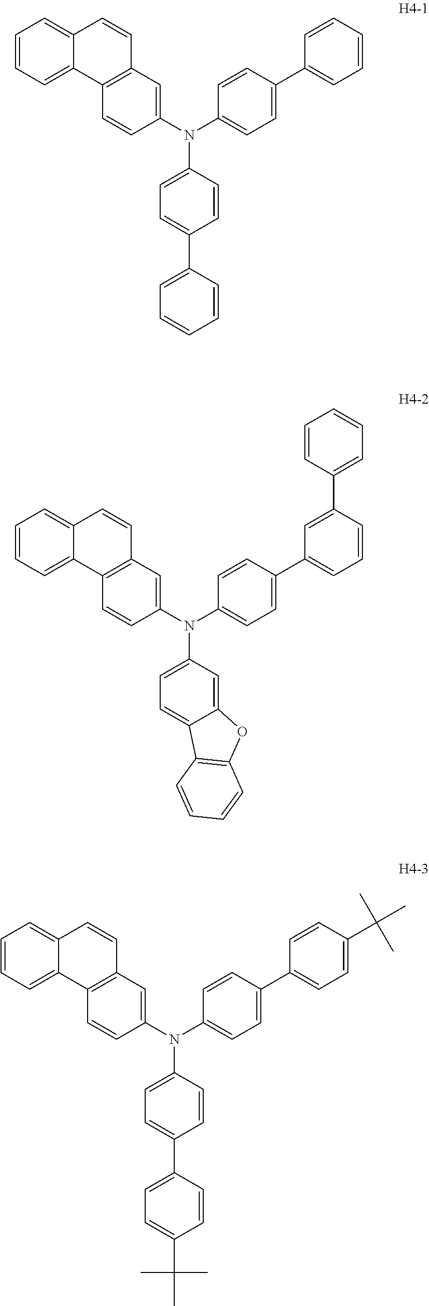 [SMILES: c1ccc(-c2ccc(N(c3ccc(-c4ccccc4)cc3)c3ccc4c(ccc5ccccc54)c3)cc2)cc1 ; c1ccc(-c2cccc(-c3ccc(N(c4ccc5c(ccc6ccccc65)c4)c4ccc5c(c4)oc4ccccc45)cc3)c2)cc1 ; CC(C)(C)c1ccc(-c2ccc(N(c3ccc(-c4ccc(C(C)(C)C)cc4)cc3)c3ccc4c(ccc5ccccc54)c3)cc2)cc1]

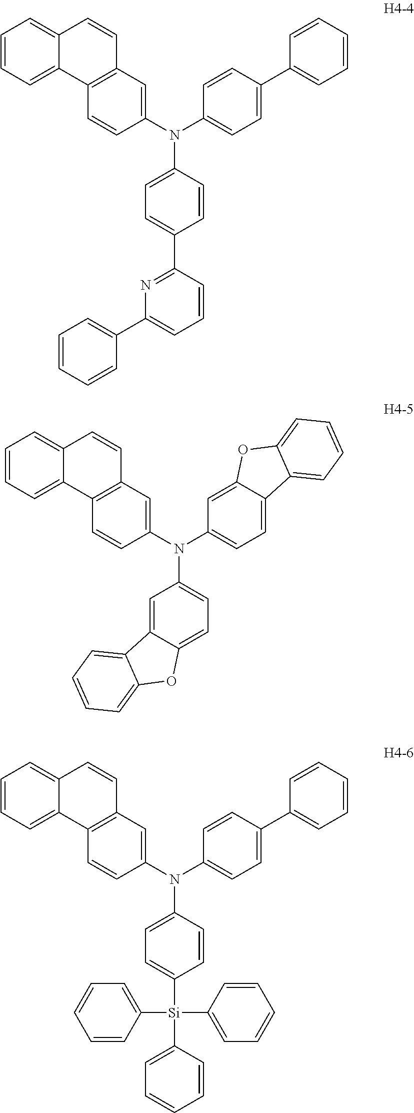 [SMILES: c1ccc(-c2ccc(N(c3ccc(-c4cccc(-c5ccccc5)n4)cc3)c3ccc4c(ccc5ccccc54)c3)cc2)cc1 ; c1ccc2c(c1)ccc1cc(N(c3ccc4c(c3)oc3ccccc34)c3ccc4oc5ccccc5c4c3)ccc12 ; c1ccc(-c2ccc(N(c3ccc([Si](c4ccccc4)(c4ccccc4)c4ccccc4)cc3)c3ccc4c(ccc5ccccc54)c3)cc2)cc1]

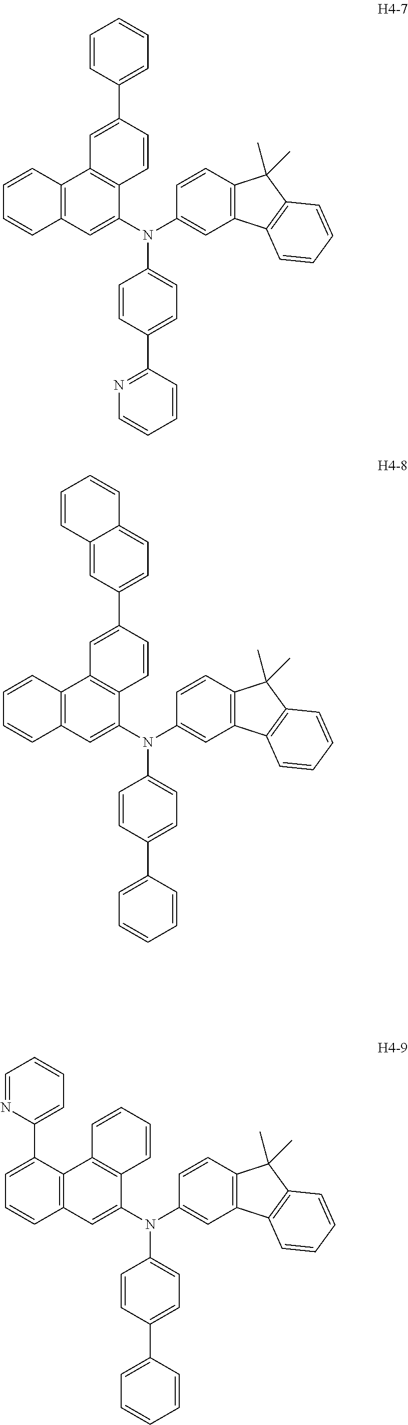 [SMILES: CC1(C)c2ccccc2-c2cc(N(c3ccc(-c4ccccn4)cc3)c3cc4ccccc4c4cc(-c5ccccc5)ccc34)ccc21 ; CC1(C)c2ccccc2-c2cc(N(c3ccc(-c4ccccc4)cc3)c3cc4ccccc4c4cc(-c5ccc6ccccc6c5)ccc34)ccc21 ; CC1(C)c2ccccc2-c2cc(N(c3ccc(-c4ccccc4)cc3)c3cc4cccc(-c5ccccn5)c4c4ccccc34)ccc21]

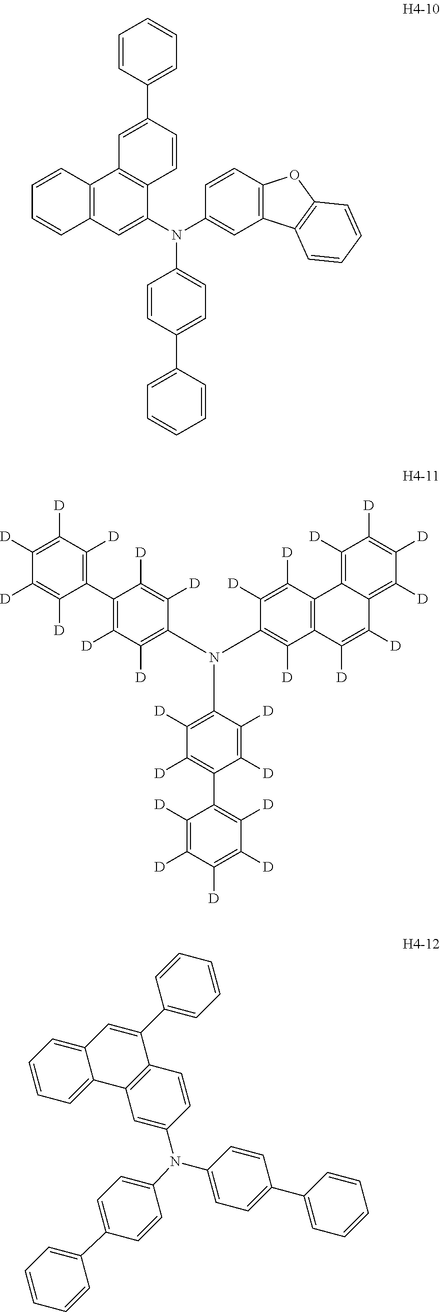 [SMILES: c1ccc(-c2ccc(N(c3ccc4oc5ccccc5c4c3)c3cc4ccccc4c4cc(-c5ccccc5)ccc34)cc2)cc1 ; [2H]c1c([2H])c([2H])c(-c2c([2H])c([2H])c(N(c3c([2H])c([2H])c(-c4c([2H])c([2H])c([2H])c([2H])c4[2H])c([2H])c3[2H])c3c([2H])c([2H])c4c(c3[2H])c([2H])c([2H])c3c([2H])c([2H])c([2H])c([2H])c34)c([2H])c2[2H])c([2H])c1[2H] ; c1ccc(-c2ccc(N(c3ccc(-c4ccccc4)cc3)c3ccc4c(-c5ccccc5)cc5ccccc5c4c3)cc2)cc1]

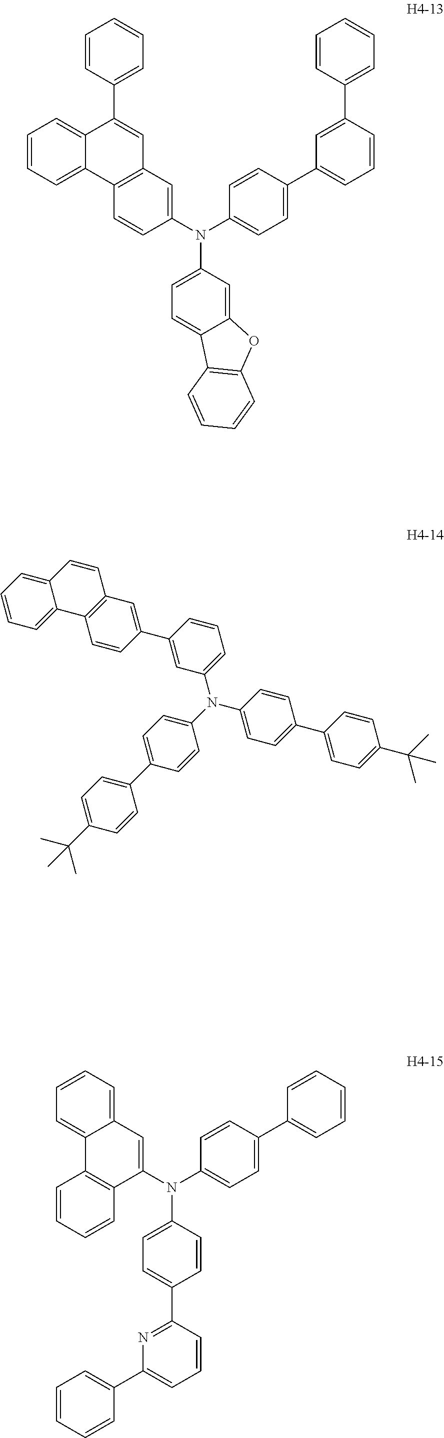 [SMILES: c1ccc(-c2cccc(-c3ccc(N(c4ccc5c(c4)cc(-c4ccccc4)c4ccccc45)c4ccc5c(c4)oc4ccccc45)cc3)c2)cc1 ; CC(C)(C)c1ccc(-c2ccc(N(c3ccc(-c4ccc(C(C)(C)C)cc4)cc3)c3cccc(-c4ccc5c(ccc6ccccc65)c4)c3)cc2)cc1 ; c1ccc(-c2ccc(N(c3ccc(-c4cccc(-c5ccccc5)n4)cc3)c3cc4ccccc4c4ccccc34)cc2)cc1]

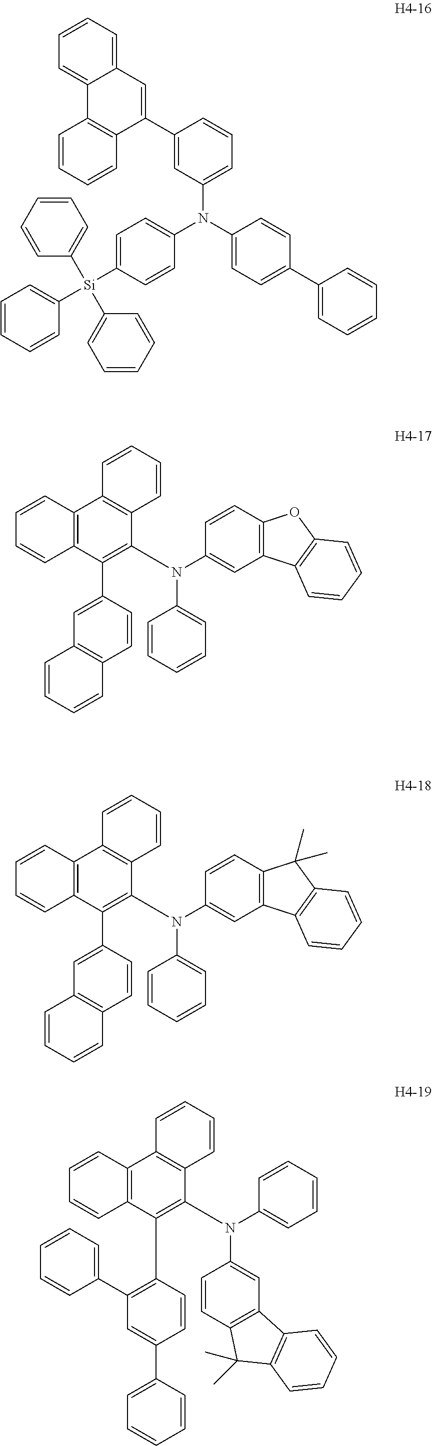 [SMILES: c1ccc(-c2ccc(N(c3ccc([Si](c4ccccc4)(c4ccccc4)c4ccccc4)cc3)c3cccc(-c4cc5ccccc5c5ccccc45)c3)cc2)cc1 ; c1ccc(N(c2ccc3oc4ccccc4c3c2)c2c(-c3ccc4ccccc4c3)c3ccccc3c3ccccc23)cc1 ; CC1(C)c2ccccc2-c2cc(N(c3ccccc3)c3c(-c4ccc5ccccc5c4)c4ccccc4c4ccccc34)ccc21 ; CC1(C)c2ccccc2-c2cc(N(c3ccccc3)c3c(-c4ccc(-c5ccccc5)cc4-c4ccccc4)c4ccccc4c4ccccc34)ccc21]

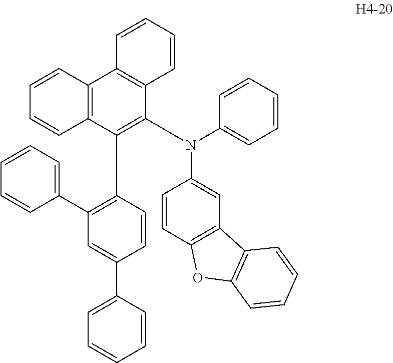 [SMILES: c1ccc(-c2ccc(-c3c(N(c4ccccc4)c4ccc5oc6ccccc6c5c4)c4ccccc4c4ccccc34)c(-c3ccccc3)c2)cc1]

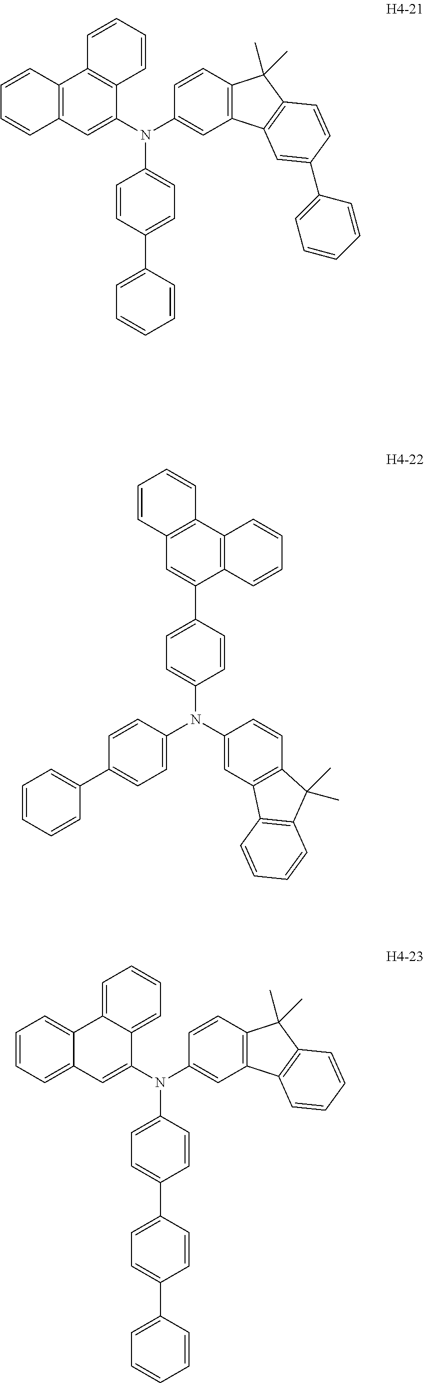 [SMILES: CC1(C)c2ccc(-c3ccccc3)cc2-c2cc(N(c3ccc(-c4ccccc4)cc3)c3cc4ccccc4c4ccccc34)ccc21 ; CC1(C)c2ccccc2-c2cc(N(c3ccc(-c4ccccc4)cc3)c3ccc(-c4cc5ccccc5c5ccccc45)cc3)ccc21 ; CC1(C)c2ccccc2-c2cc(N(c3ccc(-c4ccc(-c5ccccc5)cc4)cc3)c3cc4ccccc4c4ccccc34)ccc21]

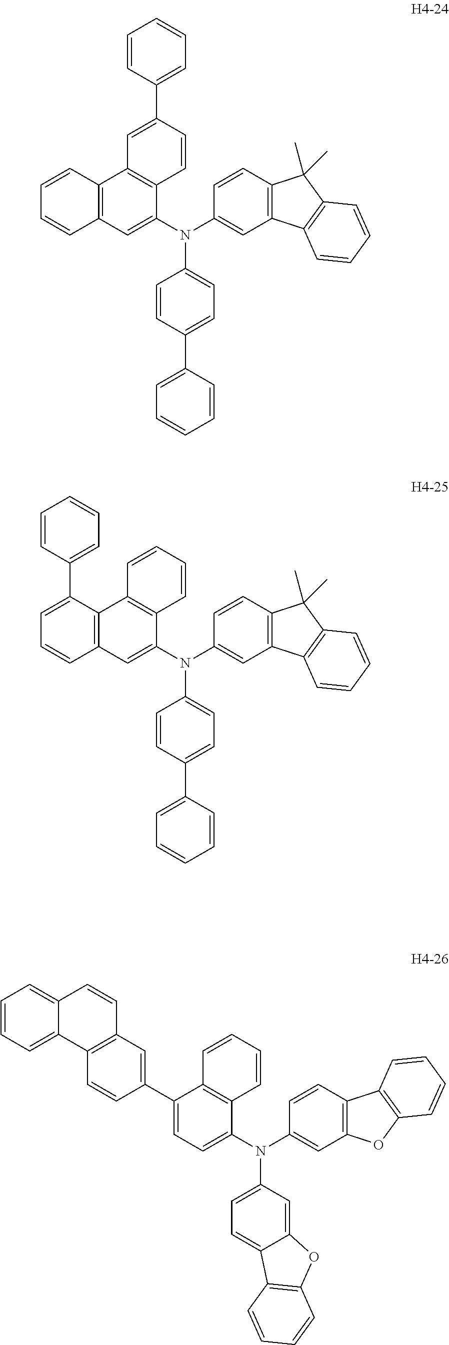 [SMILES: CC1(C)c2ccccc2-c2cc(N(c3ccc(-c4ccccc4)cc3)c3cc4ccccc4c4cc(-c5ccccc5)ccc34)ccc21 ; CC1(C)c2ccccc2-c2cc(N(c3ccc(-c4ccccc4)cc3)c3cc4cccc(-c5ccccc5)c4c4ccccc34)ccc21 ; c1ccc2c(c1)ccc1cc(-c3ccc(N(c4ccc5c(c4)oc4ccccc45)c4ccc5c(c4)oc4ccccc45)c4ccccc34)ccc12]

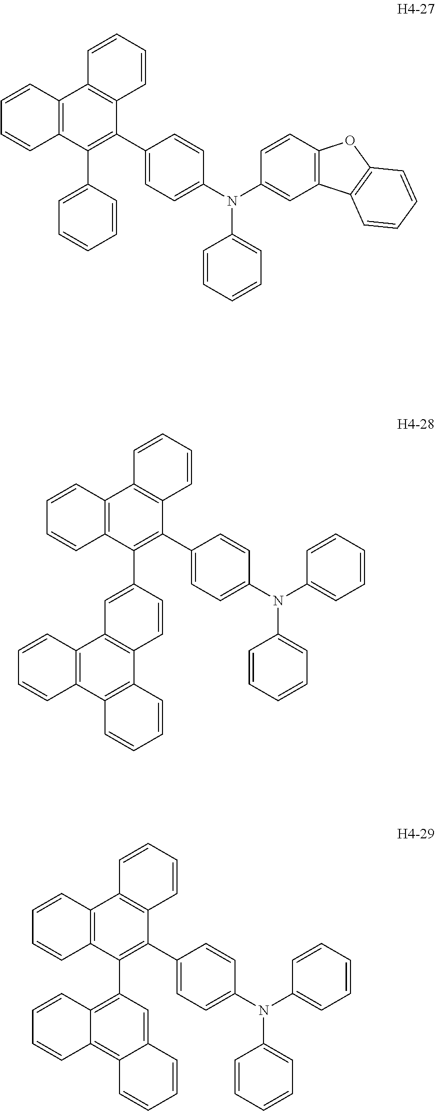 [SMILES: c1ccc(-c2c(-c3ccc(N(c4ccccc4)c4ccc5oc6ccccc6c5c4)cc3)c3ccccc3c3ccccc23)cc1 ; c1ccc(N(c2ccccc2)c2ccc(-c3c(-c4ccc5c6ccccc6c6ccccc6c5c4)c4ccccc4c4ccccc34)cc2)cc1 ; c1ccc(N(c2ccccc2)c2ccc(-c3c(-c4cc5ccccc5c5ccccc45)c4ccccc4c4ccccc34)cc2)cc1]

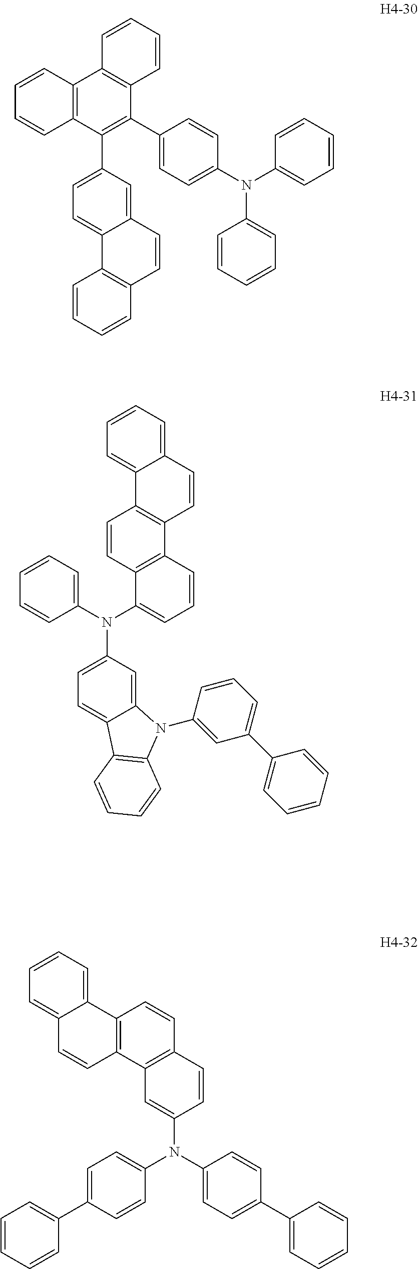 [SMILES: c1ccc(N(c2ccccc2)c2ccc(-c3c(-c4ccc5c(ccc6ccccc65)c4)c4ccccc4c4ccccc34)cc2)cc1 ; c1ccc(-c2cccc(-n3c4ccccc4c4ccc(N(c5ccccc5)c5cccc6c5ccc5c7ccccc7ccc65)cc43)c2)cc1 ; c1ccc(-c2ccc(N(c3ccc(-c4ccccc4)cc3)c3ccc4ccc5c6ccccc6ccc5c4c3)cc2)cc1]

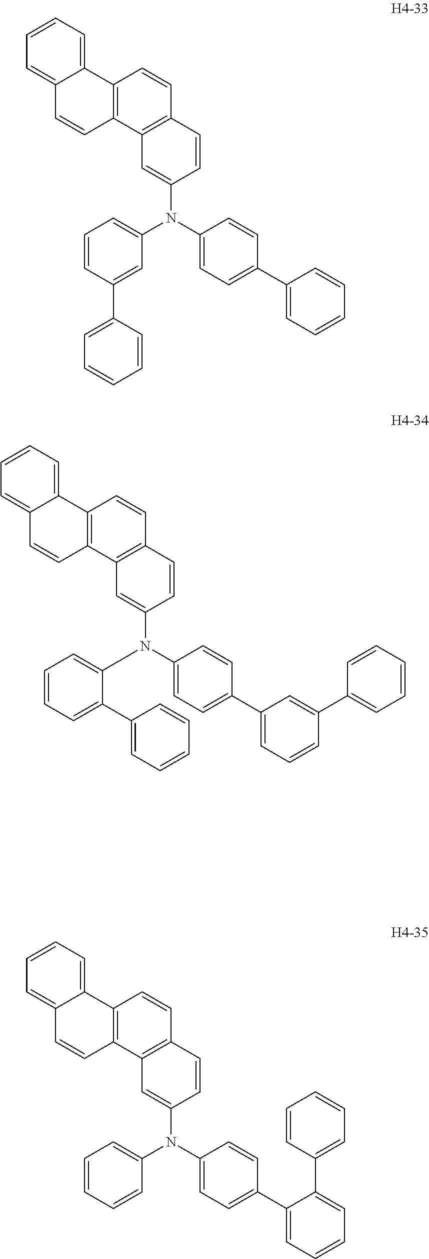 [SMILES: c1ccc(-c2ccc(N(c3cccc(-c4ccccc4)c3)c3ccc4ccc5c6ccccc6ccc5c4c3)cc2)cc1 ; c1ccc(-c2cccc(-c3ccc(N(c4ccc5ccc6c7ccccc7ccc6c5c4)c4ccccc4-c4ccccc4)cc3)c2)cc1 ; c1ccc(-c2ccccc2-c2ccc(N(c3ccccc3)c3ccc4ccc5c6ccccc6ccc5c4c3)cc2)cc1]

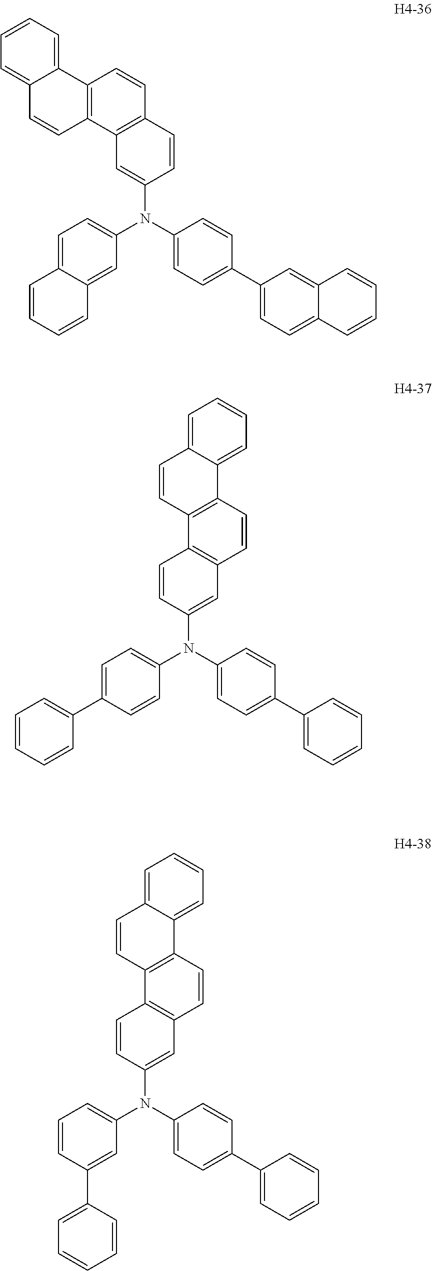 [SMILES: c1ccc2cc(-c3ccc(N(c4ccc5ccccc5c4)c4ccc5ccc6c7ccccc7ccc6c5c4)cc3)ccc2c1 ; c1ccc(-c2ccc(N(c3ccc(-c4ccccc4)cc3)c3ccc4c(ccc5c6ccccc6ccc45)c3)cc2)cc1 ; c1ccc(-c2ccc(N(c3cccc(-c4ccccc4)c3)c3ccc4c(ccc5c6ccccc6ccc45)c3)cc2)cc1]

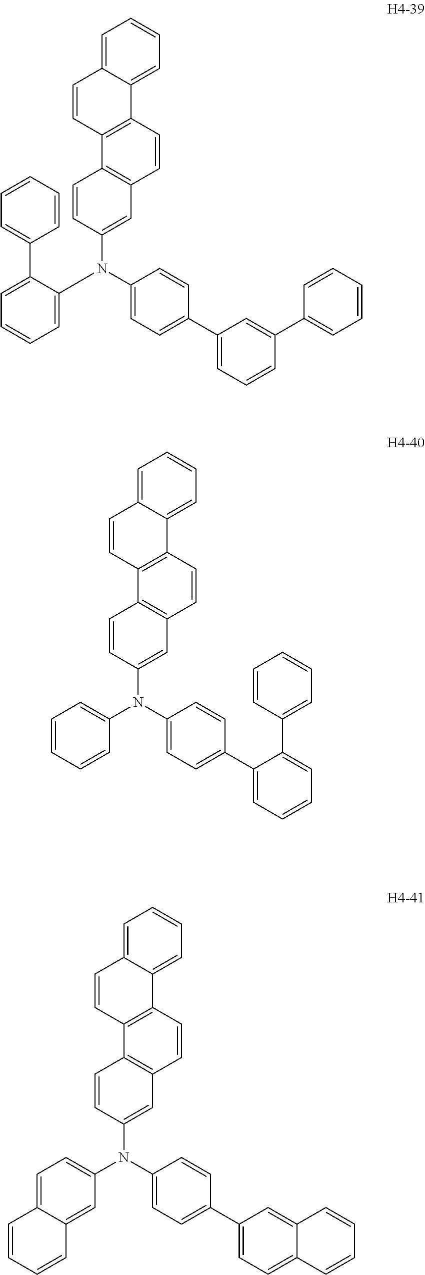 [SMILES: c1ccc(-c2cccc(-c3ccc(N(c4ccc5c(ccc6c7ccccc7ccc56)c4)c4ccccc4-c4ccccc4)cc3)c2)cc1 ; c1ccc(-c2ccccc2-c2ccc(N(c3ccccc3)c3ccc4c(ccc5c6ccccc6ccc45)c3)cc2)cc1 ; c1ccc2cc(-c3ccc(N(c4ccc5ccccc5c4)c4ccc5c(ccc6c7ccccc7ccc56)c4)cc3)ccc2c1]

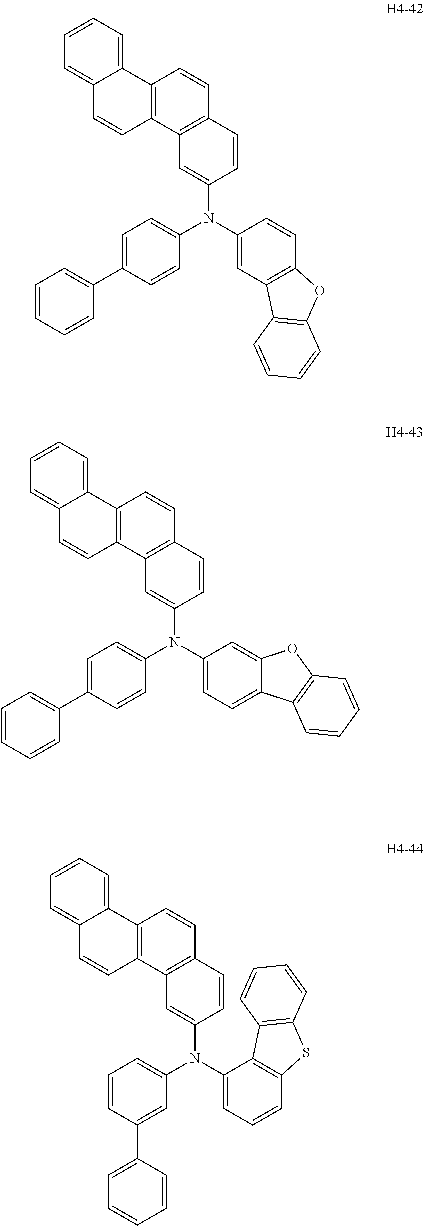 [SMILES: c1ccc(-c2ccc(N(c3ccc4ccc5c6ccccc6ccc5c4c3)c3ccc4oc5ccccc5c4c3)cc2)cc1 ; c1ccc(-c2ccc(N(c3ccc4c(c3)oc3ccccc34)c3ccc4ccc5c6ccccc6ccc5c4c3)cc2)cc1 ; c1ccc(-c2cccc(N(c3ccc4ccc5c6ccccc6ccc5c4c3)c3cccc4sc5ccccc5c34)c2)cc1]

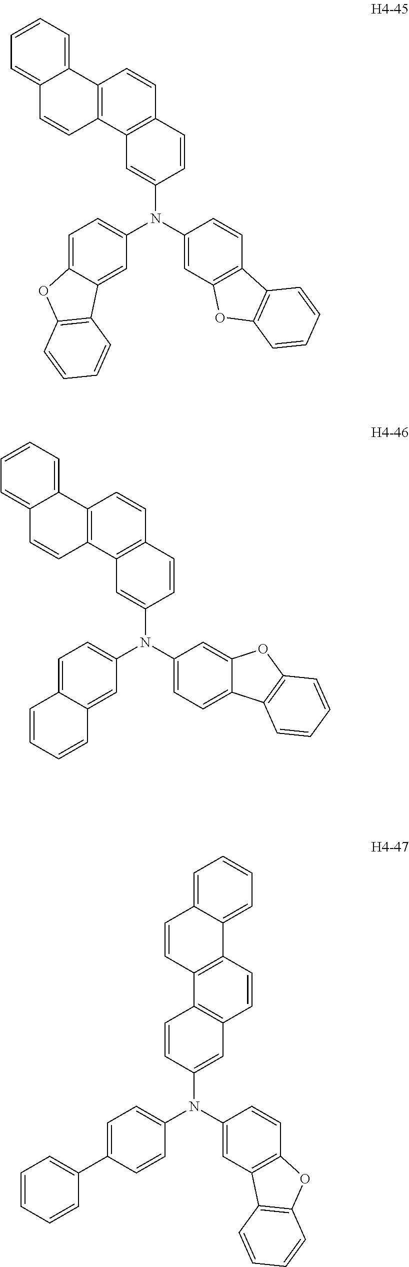 [SMILES: c1ccc2c(c1)ccc1c3cc(N(c4ccc5c(c4)oc4ccccc45)c4ccc5oc6ccccc6c5c4)ccc3ccc21 ; c1ccc2cc(N(c3ccc4c(c3)oc3ccccc34)c3ccc4ccc5c6ccccc6ccc5c4c3)ccc2c1 ; c1ccc(-c2ccc(N(c3ccc4c(ccc5c6ccccc6ccc45)c3)c3ccc4oc5ccccc5c4c3)cc2)cc1]

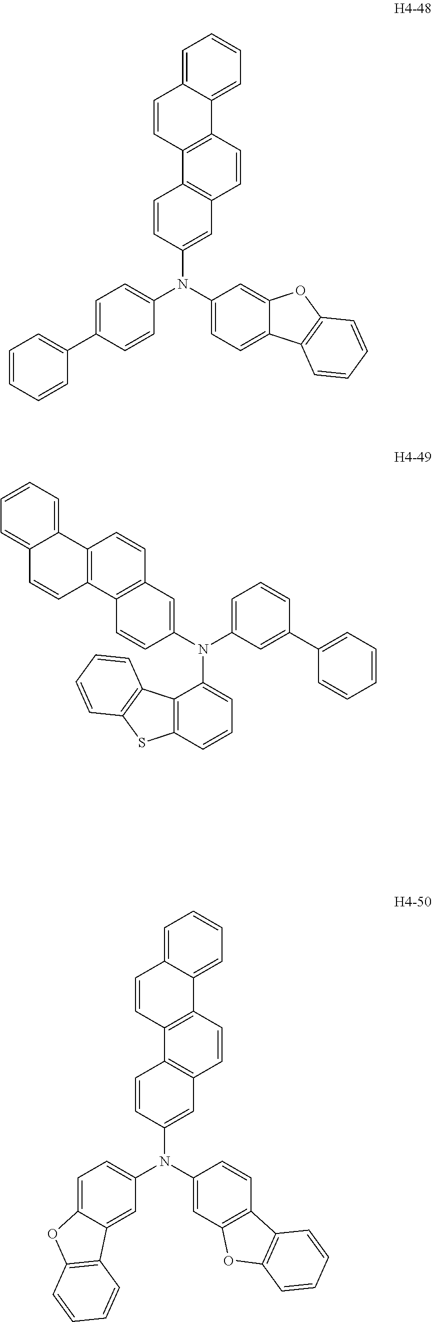 [SMILES: c1ccc(-c2ccc(N(c3ccc4c(ccc5c6ccccc6ccc45)c3)c3ccc4c(c3)oc3ccccc34)cc2)cc1 ; c1ccc(-c2cccc(N(c3ccc4c(ccc5c6ccccc6ccc45)c3)c3cccc4sc5ccccc5c34)c2)cc1 ; c1ccc2c(c1)ccc1c3ccc(N(c4ccc5c(c4)oc4ccccc45)c4ccc5oc6ccccc6c5c4)cc3ccc21]

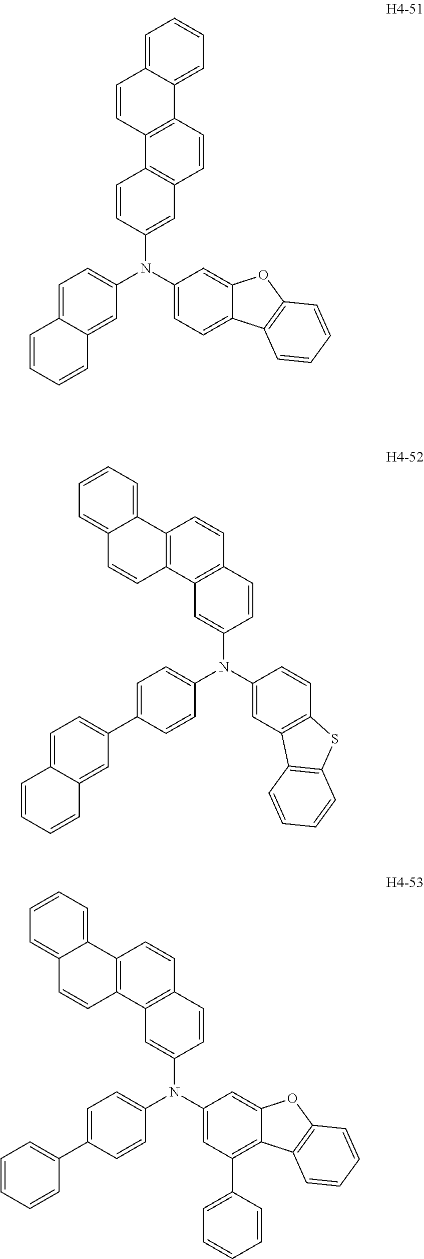 [SMILES: c1ccc2cc(N(c3ccc4c(ccc5c6ccccc6ccc45)c3)c3ccc4c(c3)oc3ccccc34)ccc2c1 ; c1ccc2cc(-c3ccc(N(c4ccc5ccc6c7ccccc7ccc6c5c4)c4ccc5sc6ccccc6c5c4)cc3)ccc2c1 ; c1ccc(-c2ccc(N(c3cc(-c4ccccc4)c4c(c3)oc3ccccc34)c3ccc4ccc5c6ccccc6ccc5c4c3)cc2)cc1]

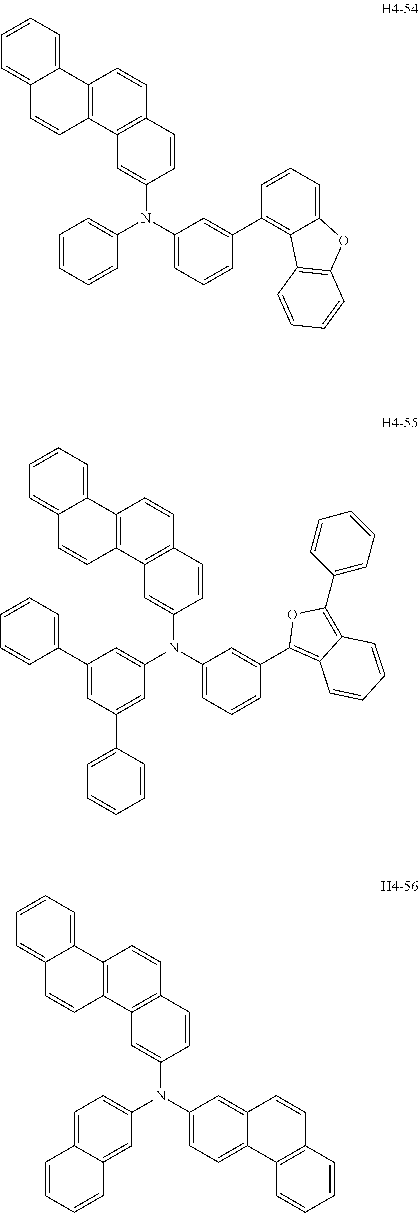 [SMILES: c1ccc(N(c2cccc(-c3cccc4oc5ccccc5c34)c2)c2ccc3ccc4c5ccccc5ccc4c3c2)cc1 ; c1ccc(-c2cc(-c3ccccc3)cc(N(c3cccc(-c4oc(-c5ccccc5)c5ccccc45)c3)c3ccc4ccc5c6ccccc6ccc5c4c3)c2)cc1 ; c1ccc2cc(N(c3ccc4c(ccc5ccccc54)c3)c3ccc4ccc5c6ccccc6ccc5c4c3)ccc2c1]

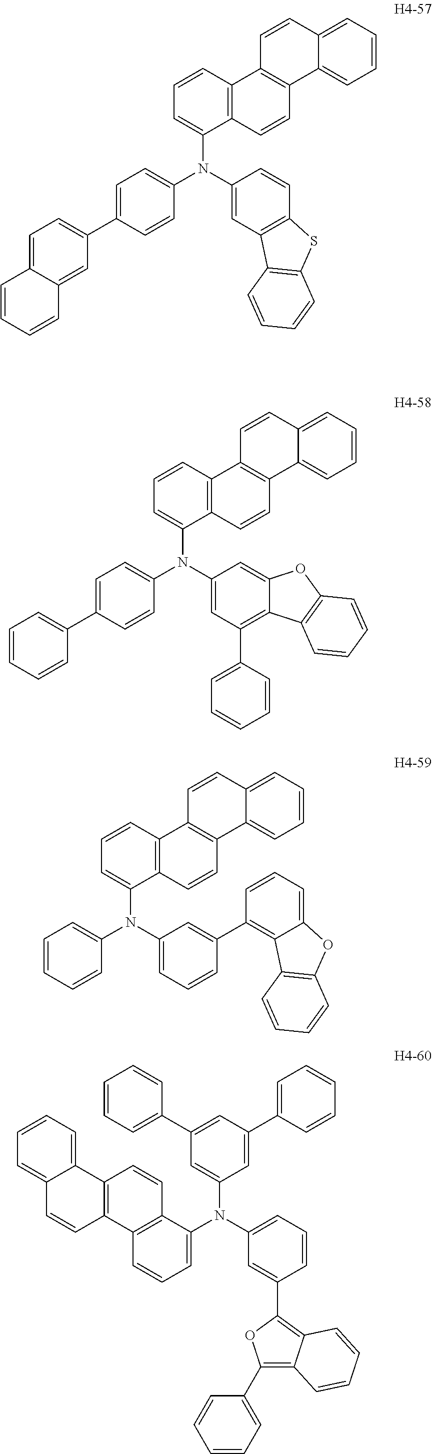

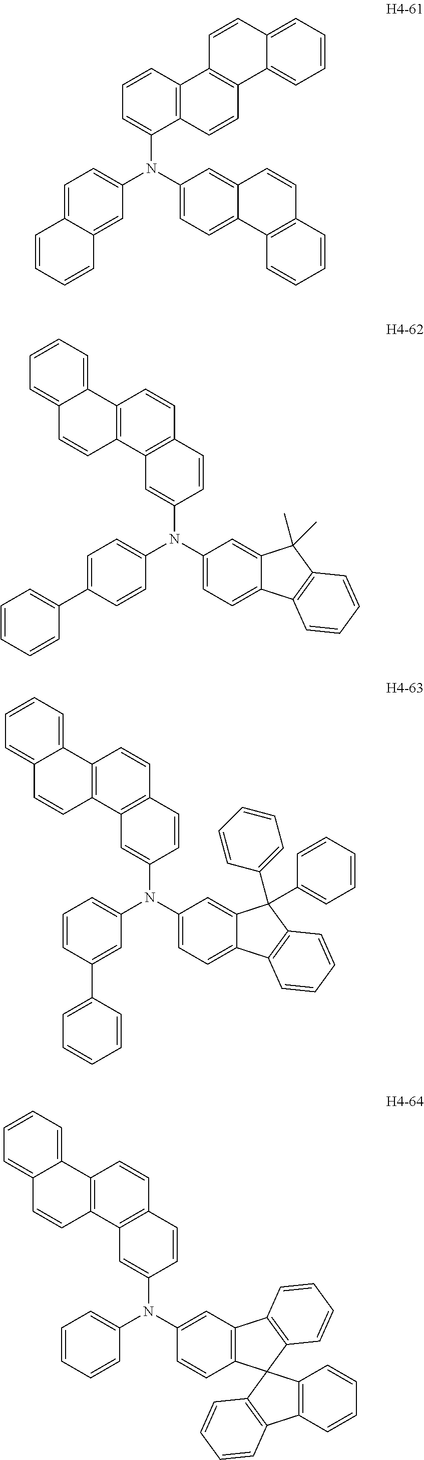

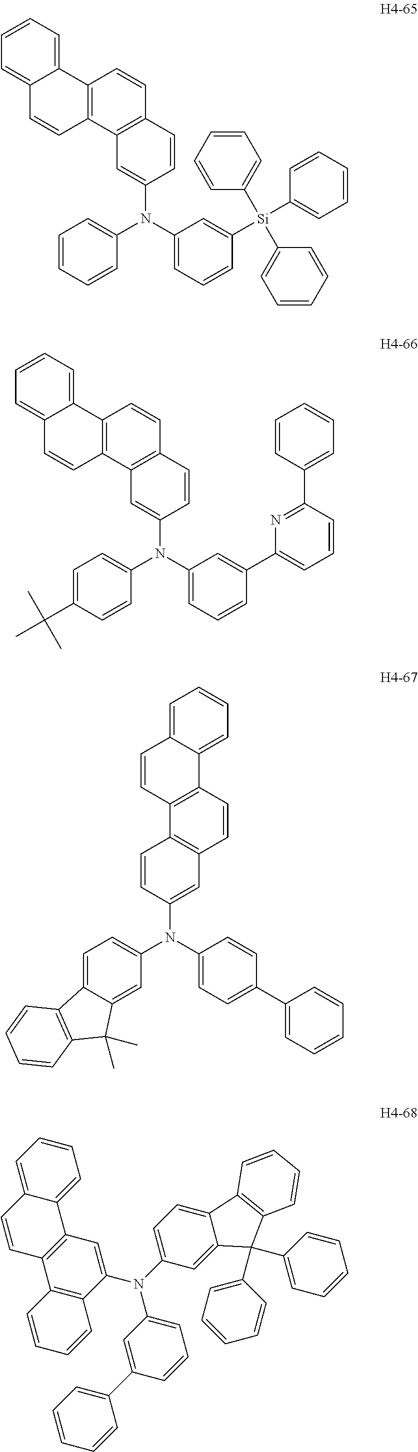 [SMILES: c1ccc(N(c2cccc([Si](c3ccccc3)(c3ccccc3)c3ccccc3)c2)c2ccc3ccc4c5ccccc5ccc4c3c2)cc1 ; CC(C)(C)c1ccc(N(c2cccc(-c3cccc(-c4ccccc4)n3)c2)c2ccc3ccc4c5ccccc5ccc4c3c2)cc1 ; CC1(C)c2ccccc2-c2ccc(N(c3ccc(-c4ccccc4)cc3)c3ccc4c(ccc5c6ccccc6ccc45)c3)cc21 ; c1ccc(-c2cccc(N(c3ccc4c(c3)C(c3ccccc3)(c3ccccc3)c3ccccc3-4)c3cc4c5ccccc5ccc4c4ccccc34)c2)cc1]

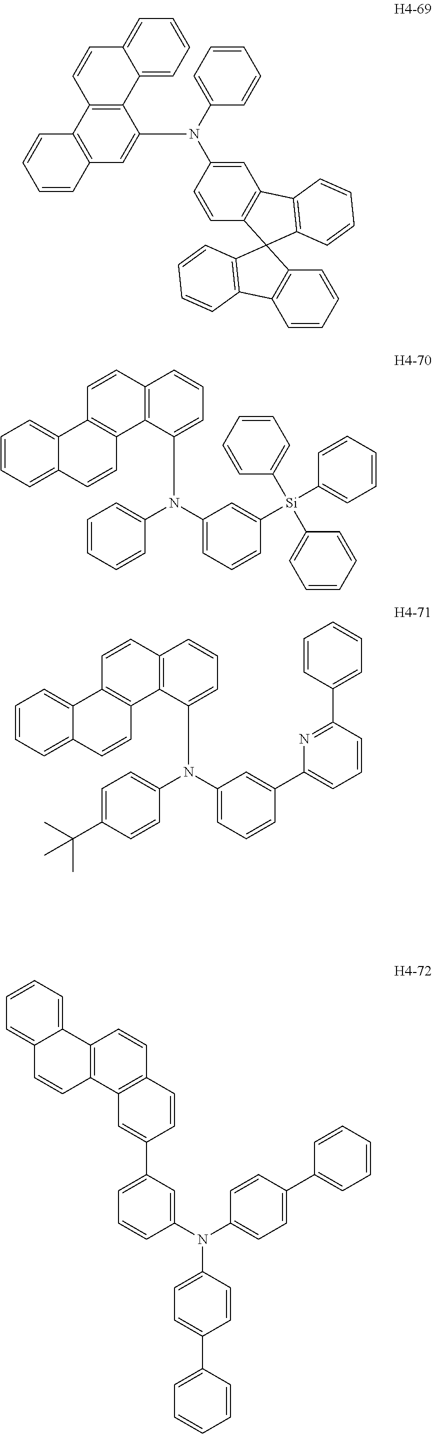

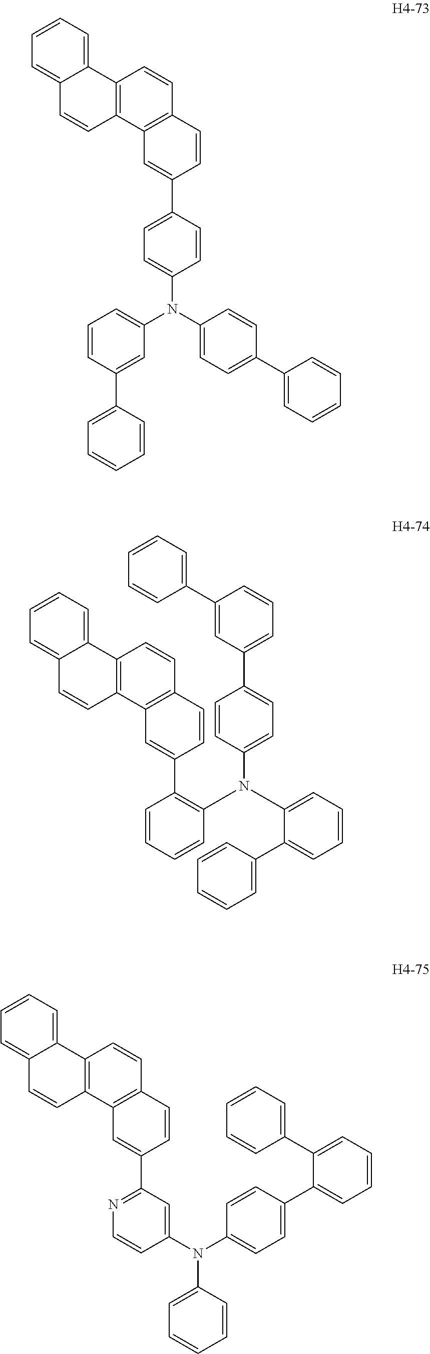 [SMILES: c1ccc(-c2ccc(N(c3ccc(-c4ccc5ccc6c7ccccc7ccc6c5c4)cc3)c3cccc(-c4ccccc4)c3)cc2)cc1 ; c1ccc(-c2cccc(-c3ccc(N(c4ccccc4-c4ccccc4)c4ccccc4-c4ccc5ccc6c7ccccc7ccc6c5c4)cc3)c2)cc1 ; c1ccc(-c2ccccc2-c2ccc(N(c3ccccc3)c3ccnc(-c4ccc5ccc6c7ccccc7ccc6c5c4)c3)cc2)cc1]

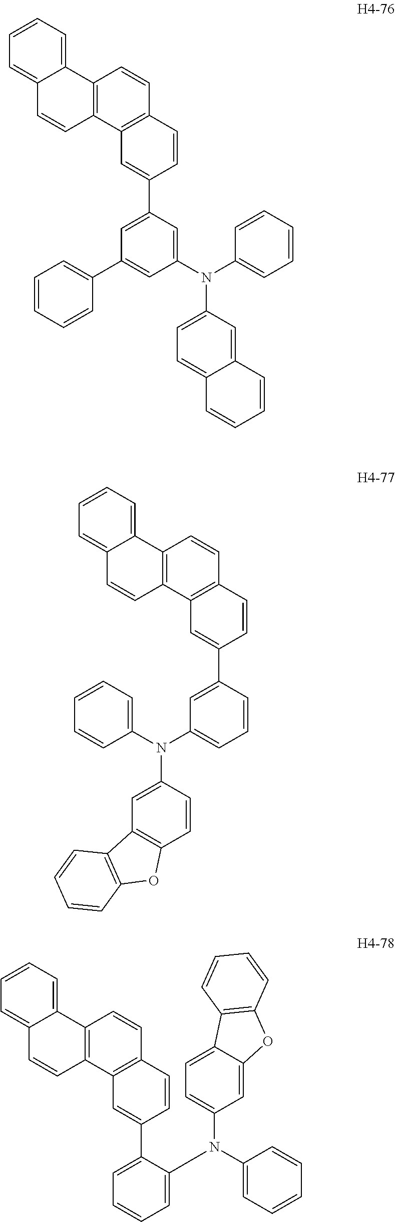 [SMILES: c1ccc(-c2cc(-c3ccc4ccc5c6ccccc6ccc5c4c3)cc(N(c3ccccc3)c3ccc4ccccc4c3)c2)cc1 ; c1ccc(N(c2cccc(-c3ccc4ccc5c6ccccc6ccc5c4c3)c2)c2ccc3oc4ccccc4c3c2)cc1 ; c1ccc(N(c2ccc3c(c2)oc2ccccc23)c2ccccc2-c2ccc3ccc4c5ccccc5ccc4c3c2)cc1]

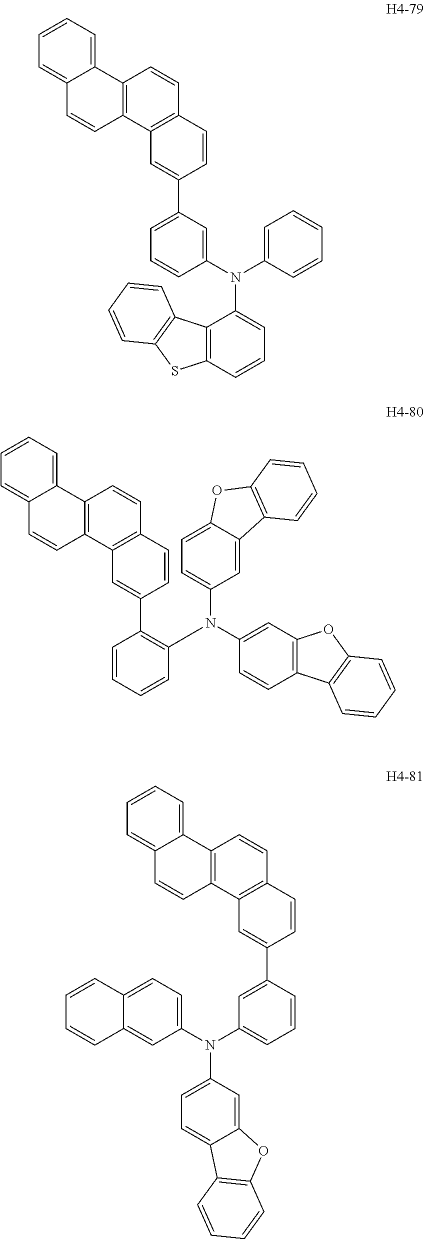 [SMILES: c1ccc(N(c2cccc(-c3ccc4ccc5c6ccccc6ccc5c4c3)c2)c2cccc3sc4ccccc4c23)cc1 ; c1ccc(N(c2ccc3c(c2)oc2ccccc23)c2ccc3oc4ccccc4c3c2)c(-c2ccc3ccc4c5ccccc5ccc4c3c2)c1 ; c1cc(-c2ccc3ccc4c5ccccc5ccc4c3c2)cc(N(c2ccc3ccccc3c2)c2ccc3c(c2)oc2ccccc23)c1]

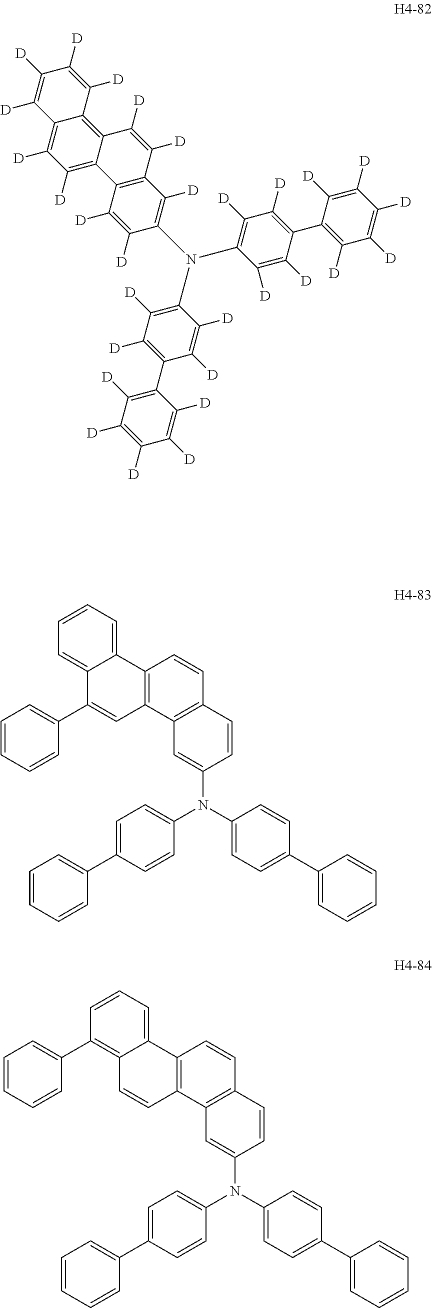 [SMILES: [2H]c1c([2H])c([2H])c(-c2c([2H])c([2H])c(N(c3c([2H])c([2H])c(-c4c([2H])c([2H])c([2H])c([2H])c4[2H])c([2H])c3[2H])c3c([2H])c([2H])c4c(c3[2H])c([2H])c([2H])c3c5c([2H])c([2H])c([2H])c([2H])c5c([2H])c([2H])c43)c([2H])c2[2H])c([2H])c1[2H] ; c1ccc(-c2ccc(N(c3ccc(-c4ccccc4)cc3)c3ccc4ccc5c6ccccc6c(-c6ccccc6)cc5c4c3)cc2)cc1 ; c1ccc(-c2ccc(N(c3ccc(-c4ccccc4)cc3)c3ccc4ccc5c6cccc(-c7ccccc7)c6ccc5c4c3)cc2)cc1]

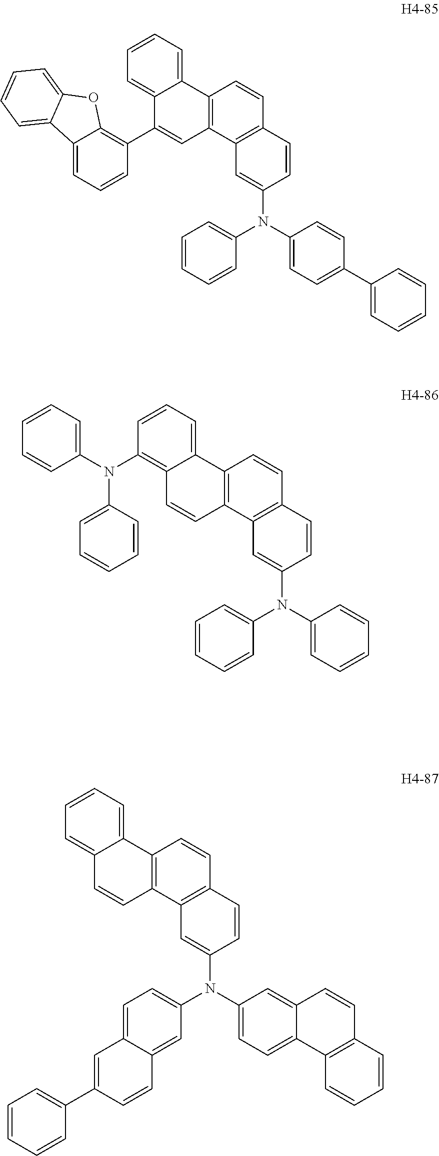 [SMILES: c1ccc(-c2ccc(N(c3ccccc3)c3ccc4ccc5c6ccccc6c(-c6cccc7c6oc6ccccc67)cc5c4c3)cc2)cc1 ; c1ccc(N(c2ccccc2)c2ccc3ccc4c5cccc(N(c6ccccc6)c6ccccc6)c5ccc4c3c2)cc1 ; c1ccc(-c2ccc3cc(N(c4ccc5c(ccc6ccccc65)c4)c4ccc5ccc6c7ccccc7ccc6c5c4)ccc3c2)cc1]

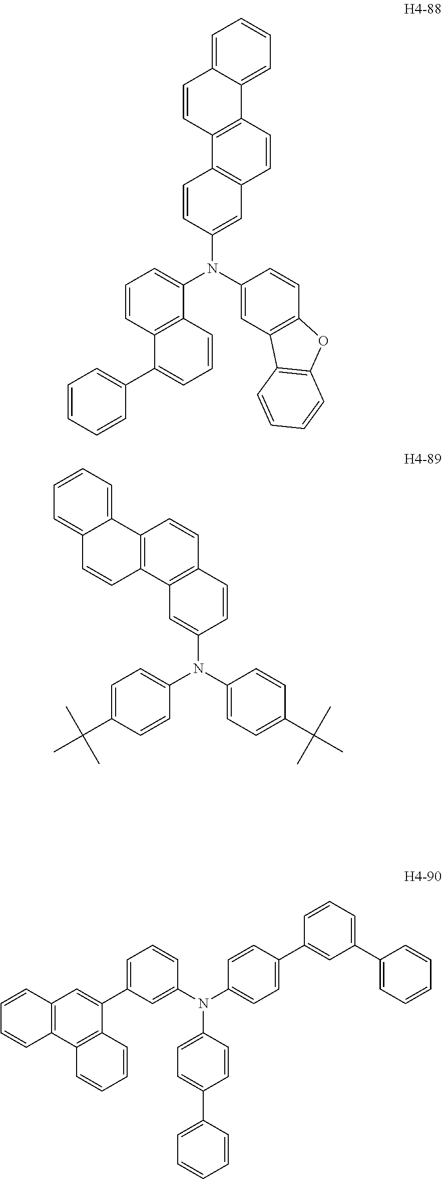

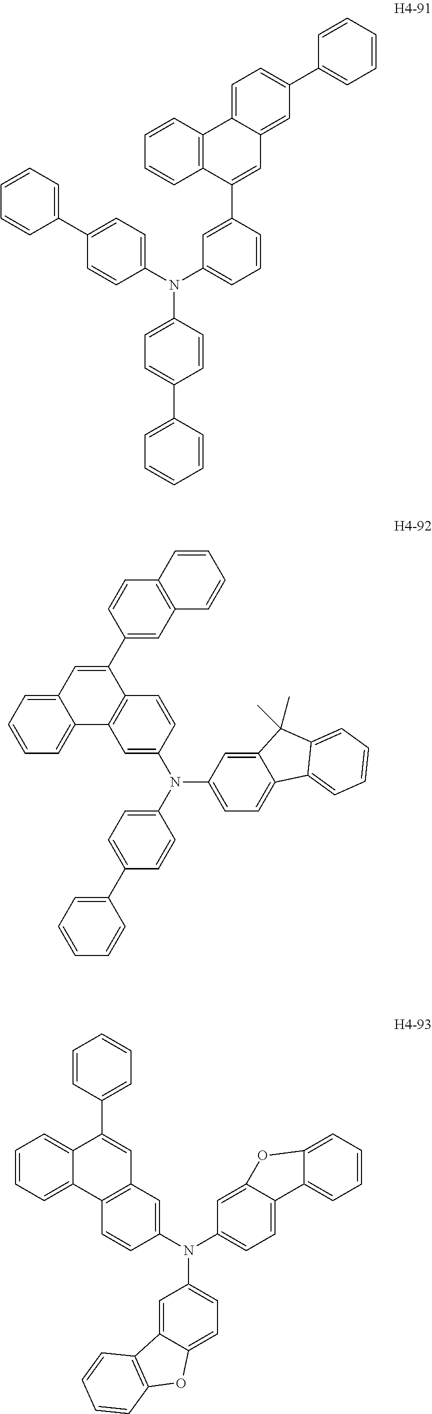 [SMILES: c1ccc(-c2ccc(N(c3ccc(-c4ccccc4)cc3)c3cccc(-c4cc5cc(-c6ccccc6)ccc5c5ccccc45)c3)cc2)cc1 ; CC1(C)c2ccccc2-c2ccc(N(c3ccc(-c4ccccc4)cc3)c3ccc4c(-c5ccc6ccccc6c5)cc5ccccc5c4c3)cc21 ; c1ccc(-c2cc3cc(N(c4ccc5c(c4)oc4ccccc45)c4ccc5oc6ccccc6c5c4)ccc3c3ccccc23)cc1]

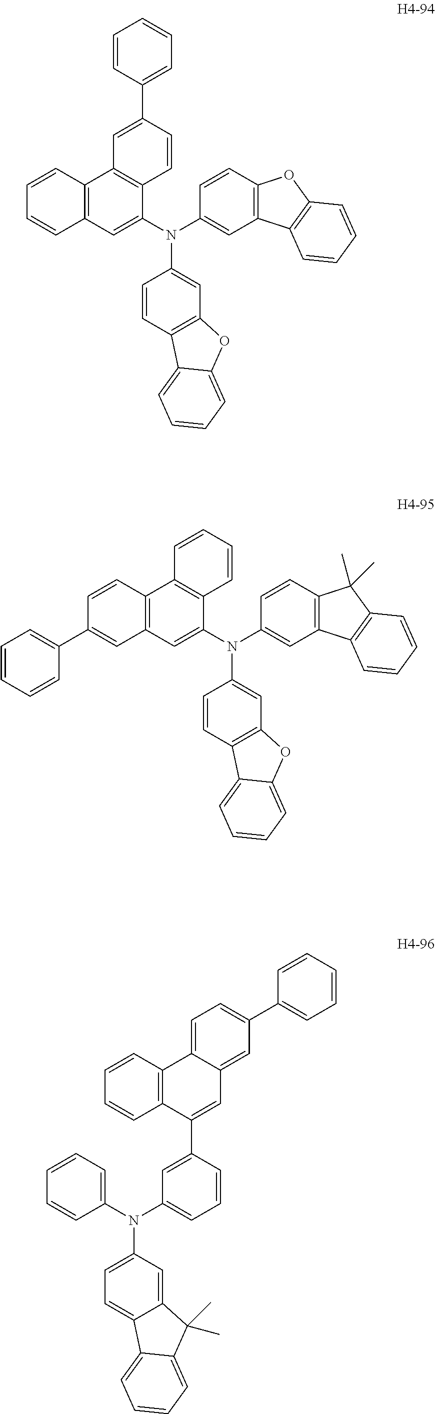 [SMILES: c1ccc(-c2ccc3c(N(c4ccc5c(c4)oc4ccccc45)c4ccc5oc6ccccc6c5c4)cc4ccccc4c3c2)cc1 ; CC1(C)c2ccccc2-c2cc(N(c3ccc4c(c3)oc3ccccc34)c3cc4cc(-c5ccccc5)ccc4c4ccccc34)ccc21 ; CC1(C)c2ccccc2-c2ccc(N(c3ccccc3)c3cccc(-c4cc5cc(-c6ccccc6)ccc5c5ccccc45)c3)cc21]

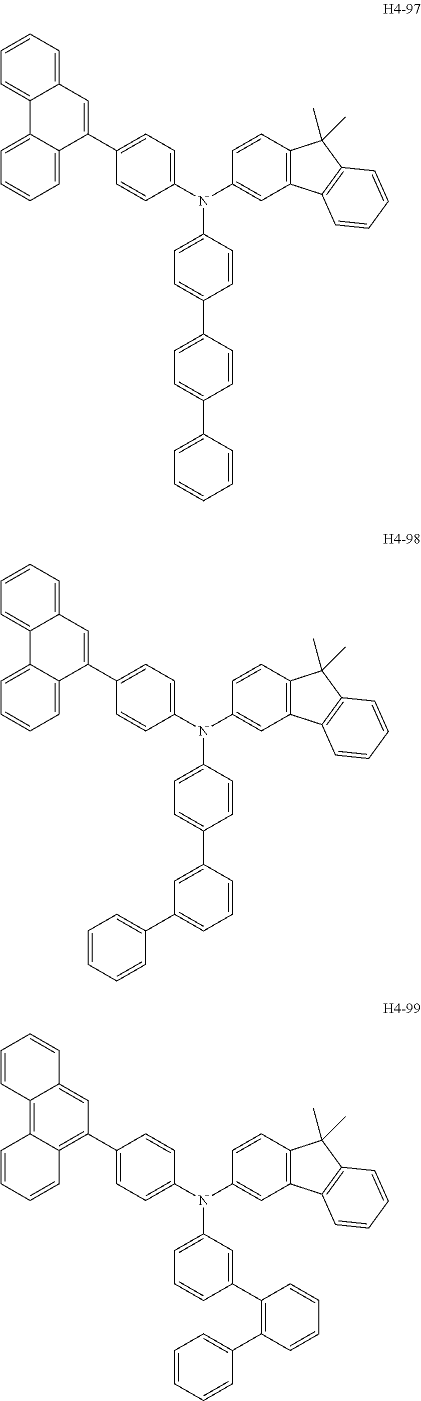 [SMILES: CC1(C)c2ccccc2-c2cc(N(c3ccc(-c4ccc(-c5ccccc5)cc4)cc3)c3ccc(-c4cc5ccccc5c5ccccc45)cc3)ccc21 ; CC1(C)c2ccccc2-c2cc(N(c3ccc(-c4cccc(-c5ccccc5)c4)cc3)c3ccc(-c4cc5ccccc5c5ccccc45)cc3)ccc21 ; CC1(C)c2ccccc2-c2cc(N(c3ccc(-c4cc5ccccc5c5ccccc45)cc3)c3cccc(-c4ccccc4-c4ccccc4)c3)ccc21]

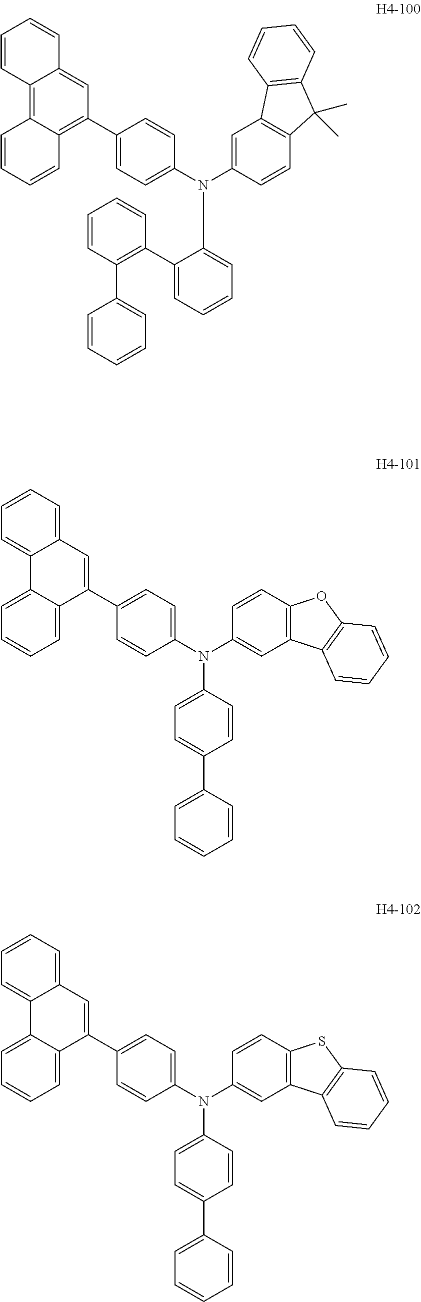 [SMILES: CC1(C)c2ccccc2-c2cc(N(c3ccc(-c4cc5ccccc5c5ccccc45)cc3)c3ccccc3-c3ccccc3-c3ccccc3)ccc21 ; c1ccc(-c2ccc(N(c3ccc(-c4cc5ccccc5c5ccccc45)cc3)c3ccc4oc5ccccc5c4c3)cc2)cc1 ; c1ccc(-c2ccc(N(c3ccc(-c4cc5ccccc5c5ccccc45)cc3)c3ccc4sc5ccccc5c4c3)cc2)cc1]

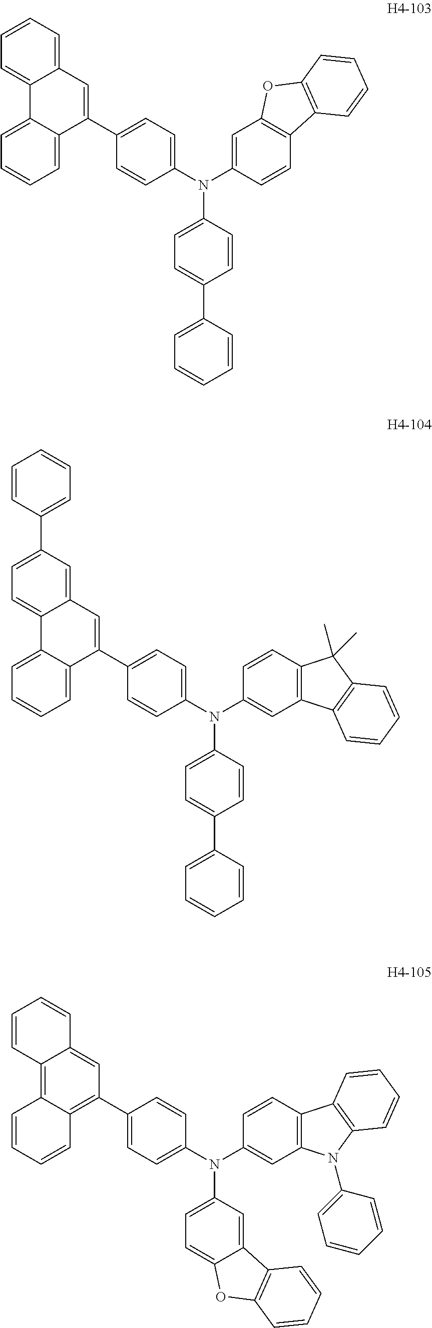 [SMILES: c1ccc(-c2ccc(N(c3ccc(-c4cc5ccccc5c5ccccc45)cc3)c3ccc4c(c3)oc3ccccc34)cc2)cc1 ; CC1(C)c2ccccc2-c2cc(N(c3ccc(-c4ccccc4)cc3)c3ccc(-c4cc5cc(-c6ccccc6)ccc5c5ccccc45)cc3)ccc21 ; c1ccc(-n2c3ccccc3c3ccc(N(c4ccc(-c5cc6ccccc6c6ccccc56)cc4)c4ccc5oc6ccccc6c5c4)cc32)cc1]

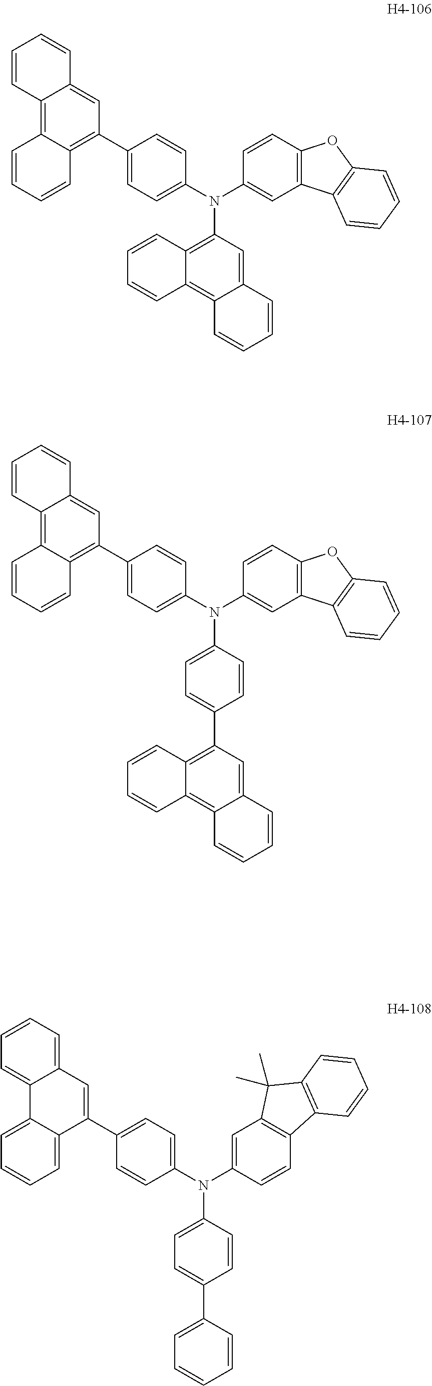 [SMILES: c1ccc2c(c1)cc(-c1ccc(N(c3ccc4oc5ccccc5c4c3)c3cc4ccccc4c4ccccc34)cc1)c1ccccc12 ; c1ccc2c(c1)cc(-c1ccc(N(c3ccc(-c4cc5ccccc5c5ccccc45)cc3)c3ccc4oc5ccccc5c4c3)cc1)c1ccccc12 ; CC1(C)c2ccccc2-c2ccc(N(c3ccc(-c4ccccc4)cc3)c3ccc(-c4cc5ccccc5c5ccccc45)cc3)cc21]

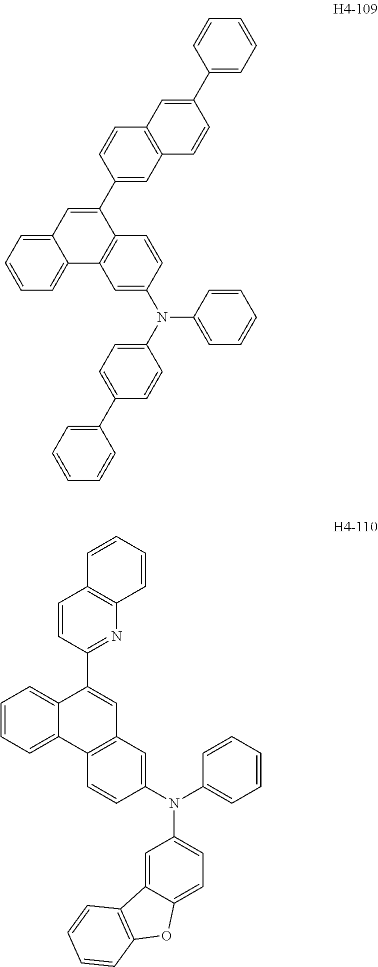 [SMILES: c1ccc(-c2ccc(N(c3ccccc3)c3ccc4c(-c5ccc6cc(-c7ccccc7)ccc6c5)cc5ccccc5c4c3)cc2)cc1 ; c1ccc(N(c2ccc3c(c2)cc(-c2ccc4ccccc4n2)c2ccccc23)c2ccc3oc4ccccc4c3c2)cc1]

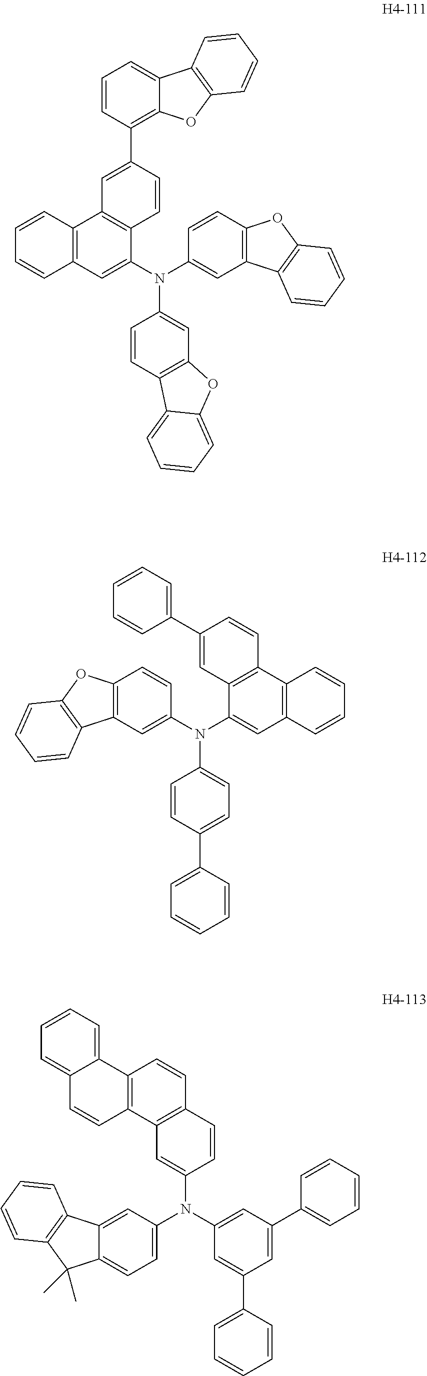 [SMILES: c1ccc2c(c1)cc(N(c1ccc3c(c1)oc1ccccc13)c1ccc3oc4ccccc4c3c1)c1ccc(-c3cccc4c3oc3ccccc34)cc12 ; c1ccc(-c2ccc(N(c3ccc4oc5ccccc5c4c3)c3cc4ccccc4c4ccc(-c5ccccc5)cc34)cc2)cc1 ; CC1(C)c2ccccc2-c2cc(N(c3cc(-c4ccccc4)cc(-c4ccccc4)c3)c3ccc4ccc5c6ccccc6ccc5c4c3)ccc21]

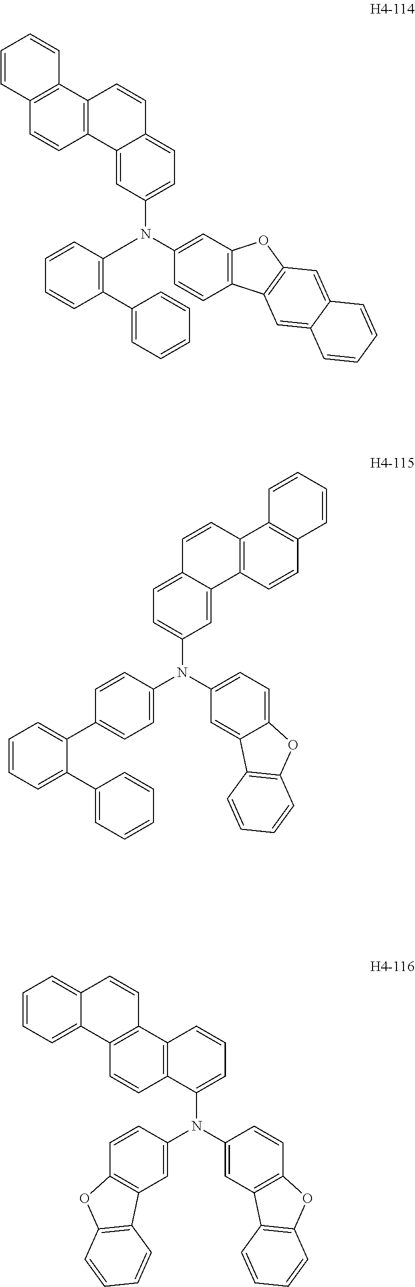 [SMILES: c1ccc(-c2ccccc2N(c2ccc3c(c2)oc2cc4ccccc4cc23)c2ccc3ccc4c5ccccc5ccc4c3c2)cc1 ; c1ccc(-c2ccccc2-c2ccc(N(c3ccc4ccc5c6ccccc6ccc5c4c3)c3ccc4oc5ccccc5c4c3)cc2)cc1 ; c1ccc2c(c1)ccc1c3cccc(N(c4ccc5oc6ccccc6c5c4)c4ccc5oc6ccccc6c5c4)c3ccc21]

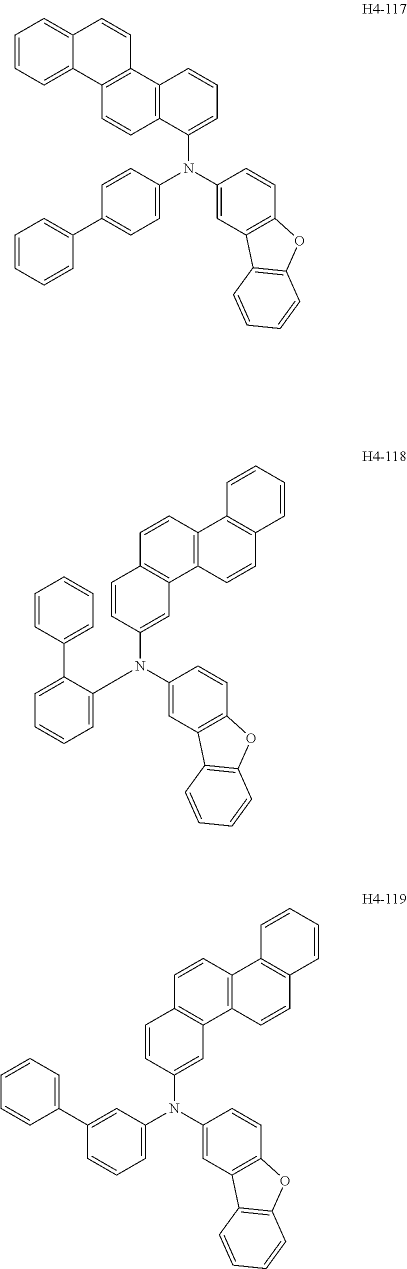 [SMILES: c1ccc(-c2ccc(N(c3ccc4oc5ccccc5c4c3)c3cccc4c3ccc3c5ccccc5ccc43)cc2)cc1 ; c1ccc(-c2ccccc2N(c2ccc3ccc4c5ccccc5ccc4c3c2)c2ccc3oc4ccccc4c3c2)cc1 ; c1ccc(-c2cccc(N(c3ccc4ccc5c6ccccc6ccc5c4c3)c3ccc4oc5ccccc5c4c3)c2)cc1]

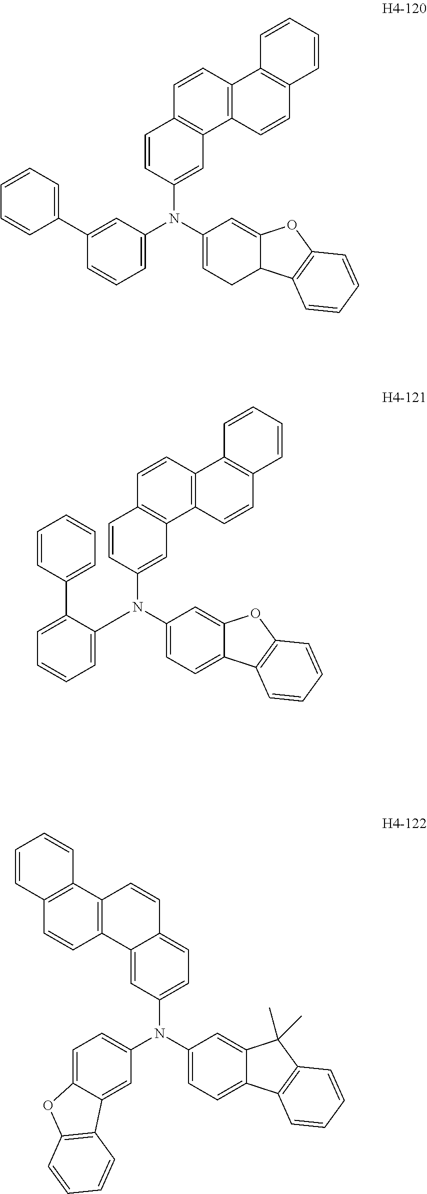 [SMILES: C1=C2Oc3ccccc3C2CC=C1N(c1cccc(-c2ccccc2)c1)c1ccc2ccc3c4ccccc4ccc3c2c1 ; c1ccc(-c2ccccc2N(c2ccc3c(c2)oc2ccccc23)c2ccc3ccc4c5ccccc5ccc4c3c2)cc1 ; CC1(C)c2ccccc2-c2ccc(N(c3ccc4ccc5c6ccccc6ccc5c4c3)c3ccc4oc5ccccc5c4c3)cc21]

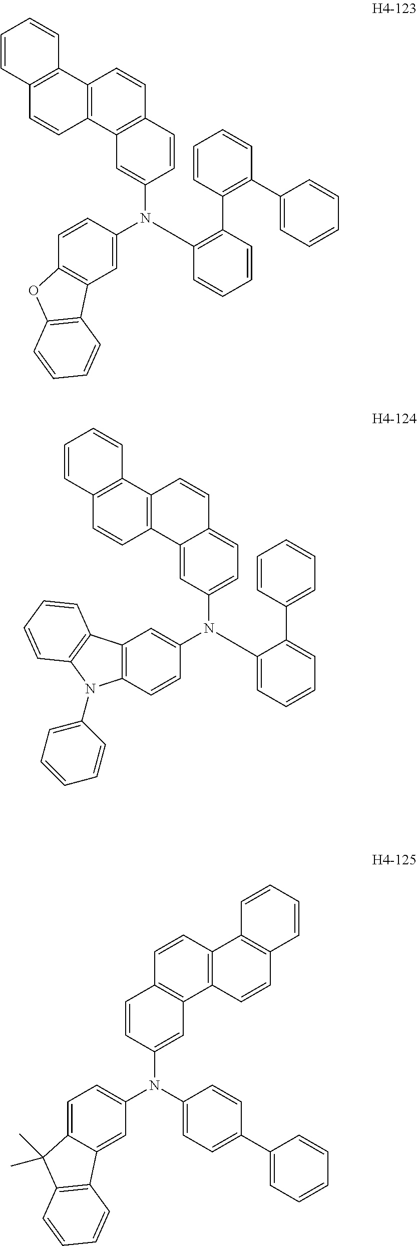 [SMILES: c1ccc(-c2ccccc2-c2ccccc2N(c2ccc3ccc4c5ccccc5ccc4c3c2)c2ccc3oc4ccccc4c3c2)cc1 ; c1ccc(-c2ccccc2N(c2ccc3ccc4c5ccccc5ccc4c3c2)c2ccc3c(c2)c2ccccc2n3-c2ccccc2)cc1 ; CC1(C)c2ccccc2-c2cc(N(c3ccc(-c4ccccc4)cc3)c3ccc4ccc5c6ccccc6ccc5c4c3)ccc21]

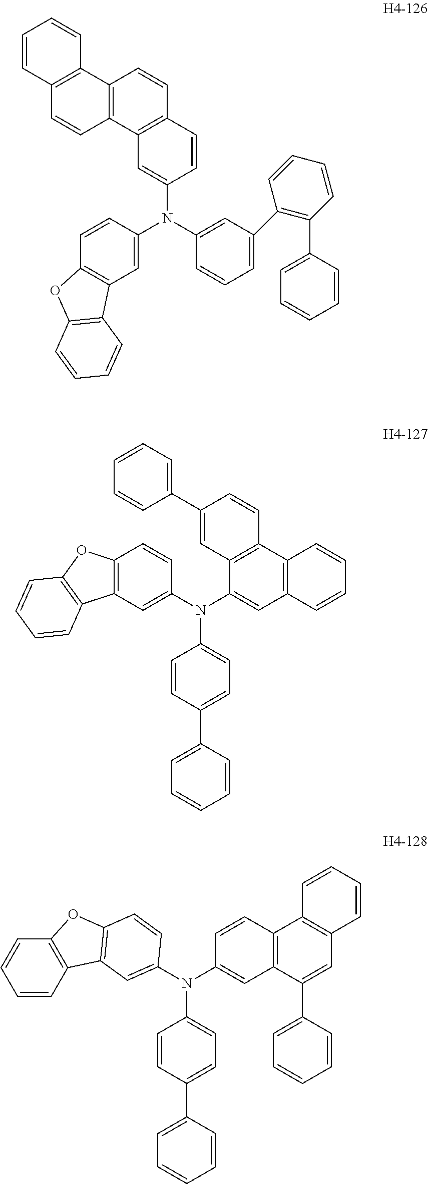 [SMILES: c1ccc(-c2ccccc2-c2cccc(N(c3ccc4ccc5c6ccccc6ccc5c4c3)c3ccc4oc5ccccc5c4c3)c2)cc1 ; c1ccc(-c2ccc(N(c3ccc4oc5ccccc5c4c3)c3cc4ccccc4c4ccc(-c5ccccc5)cc34)cc2)cc1 ; c1ccc(-c2ccc(N(c3ccc4c(c3)c(-c3ccccc3)cc3ccccc34)c3ccc4oc5ccccc5c4c3)cc2)cc1]

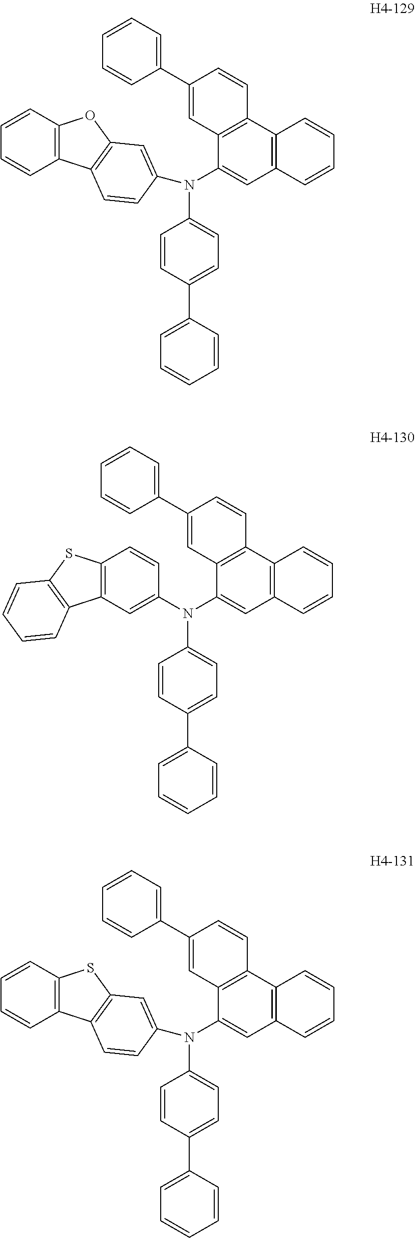 [SMILES: c1ccc(-c2ccc(N(c3ccc4c(c3)oc3ccccc34)c3cc4ccccc4c4ccc(-c5ccccc5)cc34)cc2)cc1 ; c1ccc(-c2ccc(N(c3ccc4sc5ccccc5c4c3)c3cc4ccccc4c4ccc(-c5ccccc5)cc34)cc2)cc1 ; c1ccc(-c2ccc(N(c3ccc4c(c3)sc3ccccc34)c3cc4ccccc4c4ccc(-c5ccccc5)cc34)cc2)cc1]

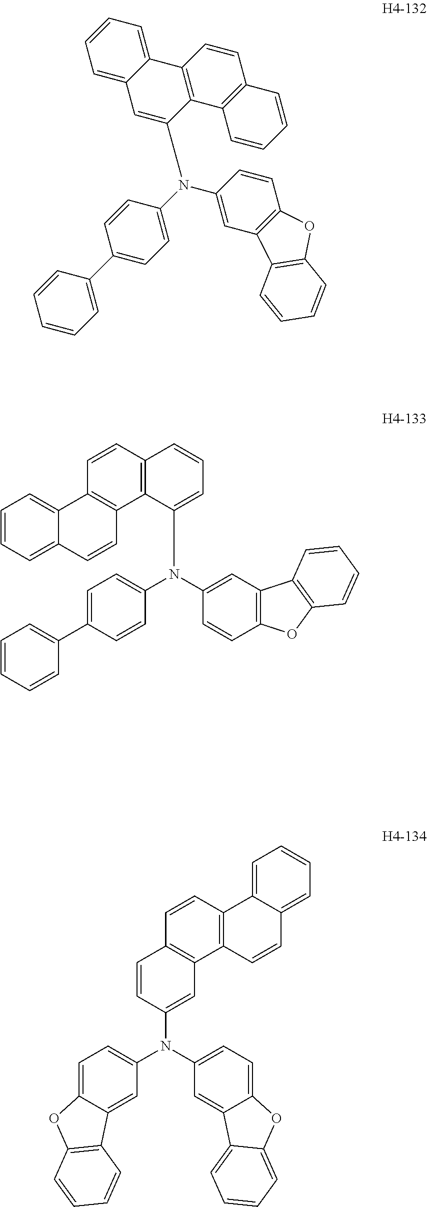 [SMILES: c1ccc(-c2ccc(N(c3ccc4oc5ccccc5c4c3)c3cc4ccccc4c4ccc5ccccc5c34)cc2)cc1 ; c1ccc(-c2ccc(N(c3ccc4oc5ccccc5c4c3)c3cccc4ccc5c6ccccc6ccc5c34)cc2)cc1 ; c1ccc2c(c1)ccc1c3cc(N(c4ccc5oc6ccccc6c5c4)c4ccc5oc6ccccc6c5c4)ccc3ccc21]

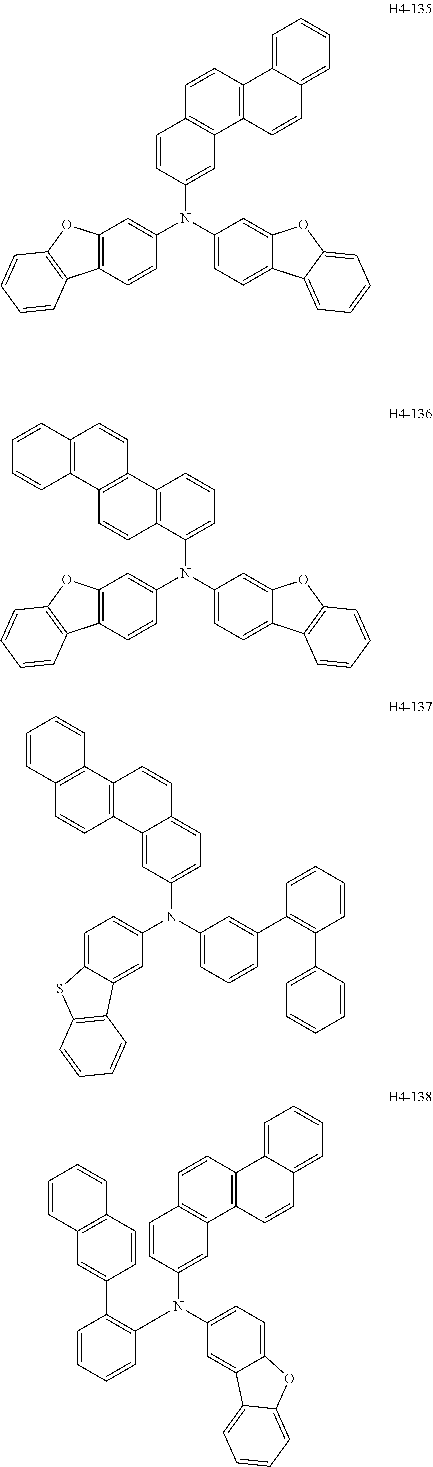 [SMILES: c1ccc2c(c1)ccc1c3cc(N(c4ccc5c(c4)oc4ccccc45)c4ccc5c(c4)oc4ccccc45)ccc3ccc21 ; c1ccc2c(c1)ccc1c3cccc(N(c4ccc5c(c4)oc4ccccc45)c4ccc5c(c4)oc4ccccc45)c3ccc21 ; c1ccc(-c2ccccc2-c2cccc(N(c3ccc4ccc5c6ccccc6ccc5c4c3)c3ccc4sc5ccccc5c4c3)c2)cc1 ; c1ccc(N(c2ccc3ccc4c5ccccc5ccc4c3c2)c2ccc3oc4ccccc4c3c2)c(-c2ccc3ccccc3c2)c1]

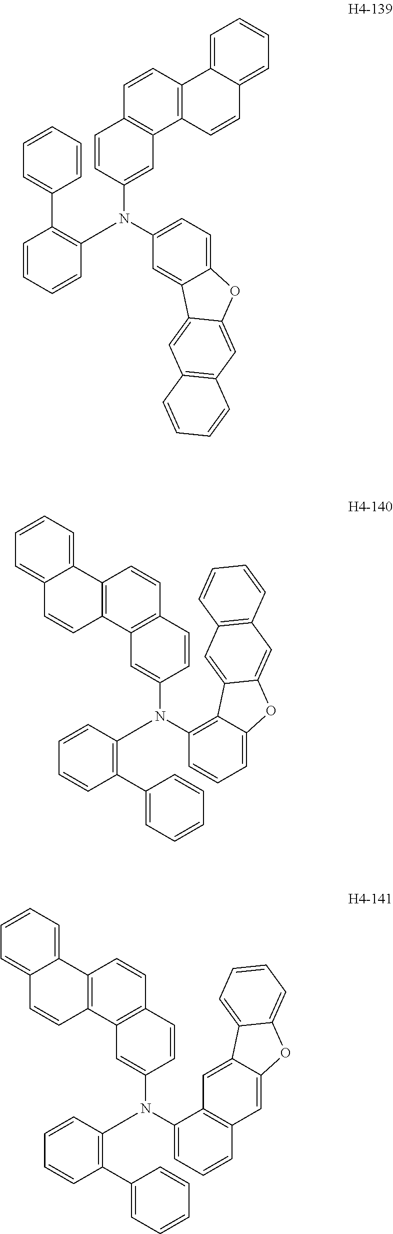 [SMILES: c1ccc(-c2ccccc2N(c2ccc3ccc4c5ccccc5ccc4c3c2)c2ccc3oc4cc5ccccc5cc4c3c2)cc1 ; c1ccc(-c2ccccc2N(c2ccc3ccc4c5ccccc5ccc4c3c2)c2cccc3oc4cc5ccccc5cc4c23)cc1 ; c1ccc(-c2ccccc2N(c2ccc3ccc4c5ccccc5ccc4c3c2)c2cccc3cc4oc5ccccc5c4cc23)cc1]

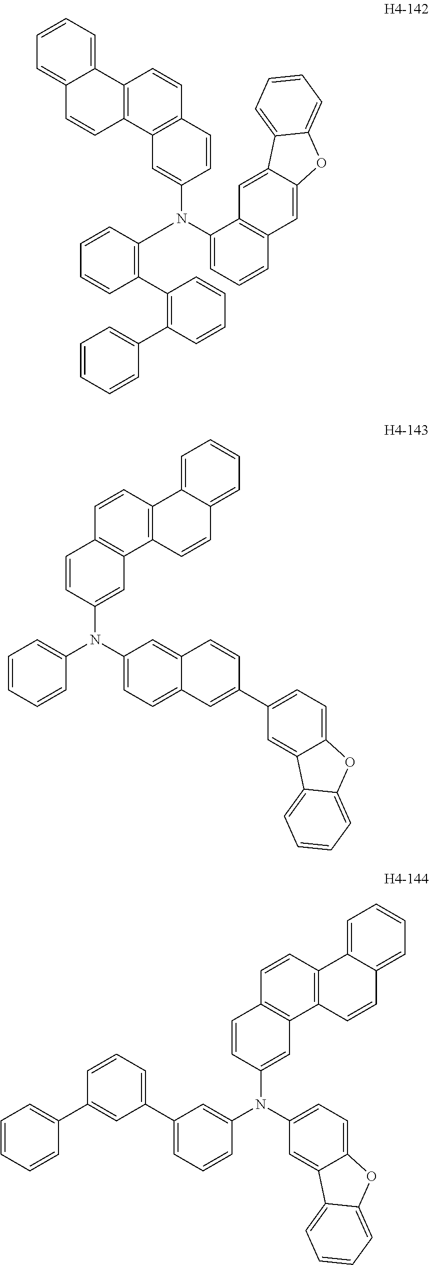 [SMILES: c1ccc(-c2ccccc2-c2ccccc2N(c2ccc3ccc4c5ccccc5ccc4c3c2)c2cccc3cc4oc5ccccc5c4cc23)cc1 ; c1ccc(N(c2ccc3cc(-c4ccc5oc6ccccc6c5c4)ccc3c2)c2ccc3ccc4c5ccccc5ccc4c3c2)cc1 ; c1ccc(-c2cccc(-c3cccc(N(c4ccc5ccc6c7ccccc7ccc6c5c4)c4ccc5oc6ccccc6c5c4)c3)c2)cc1]

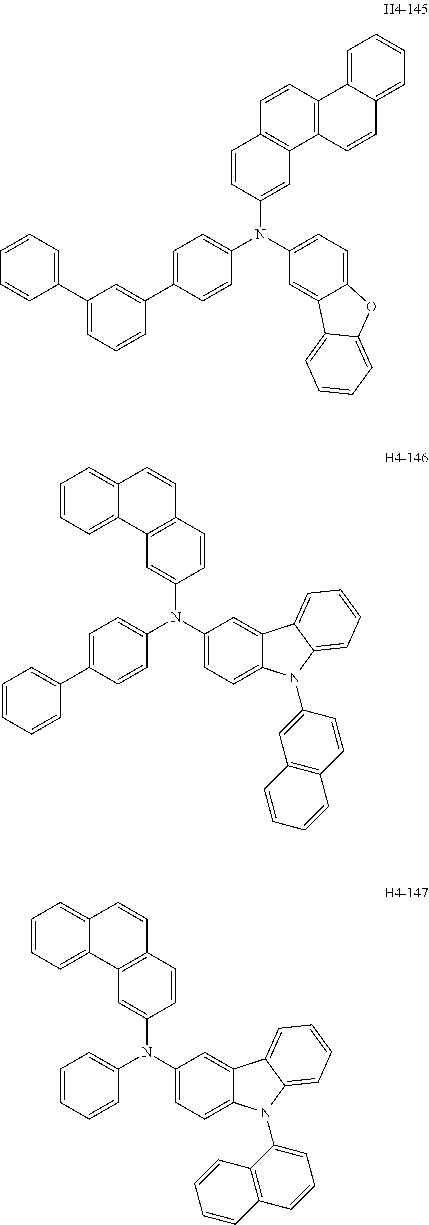 [SMILES: c1ccc(-c2cccc(-c3ccc(N(c4ccc5ccc6c7ccccc7ccc6c5c4)c4ccc5oc6ccccc6c5c4)cc3)c2)cc1 ; c1ccc(-c2ccc(N(c3ccc4ccc5ccccc5c4c3)c3ccc4c(c3)c3ccccc3n4-c3ccc4ccccc4c3)cc2)cc1 ; c1ccc(N(c2ccc3ccc4ccccc4c3c2)c2ccc3c(c2)c2ccccc2n3-c2cccc3ccccc23)cc1]

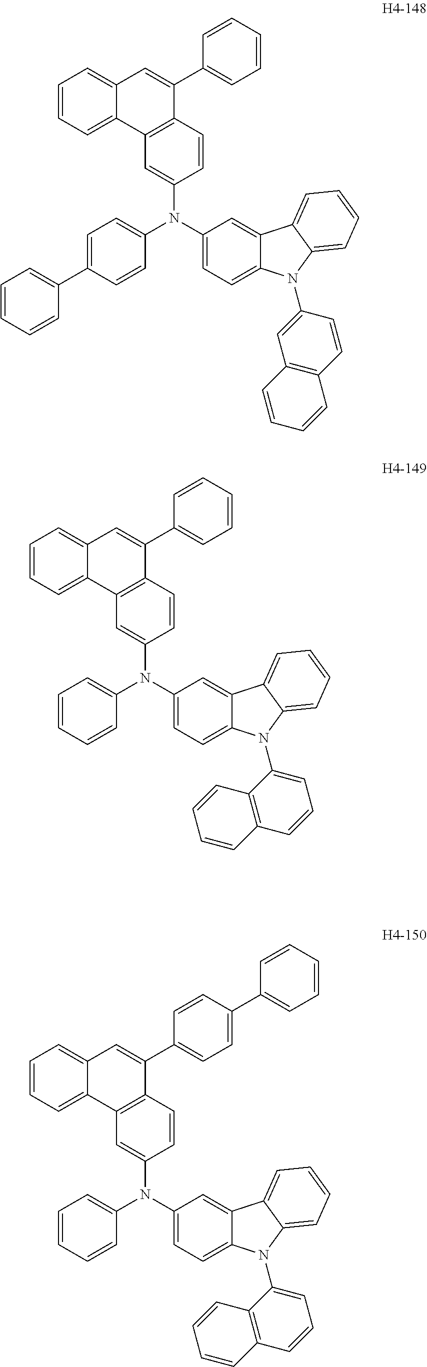 [SMILES: c1ccc(-c2ccc(N(c3ccc4c(-c5ccccc5)cc5ccccc5c4c3)c3ccc4c(c3)c3ccccc3n4-c3ccc4ccccc4c3)cc2)cc1 ; c1ccc(-c2cc3ccccc3c3cc(N(c4ccccc4)c4ccc5c(c4)c4ccccc4n5-c4cccc5ccccc45)ccc23)cc1 ; c1ccc(-c2ccc(-c3cc4ccccc4c4cc(N(c5ccccc5)c5ccc6c(c5)c5ccccc5n6-c5cccc6ccccc56)ccc34)cc2)cc1]

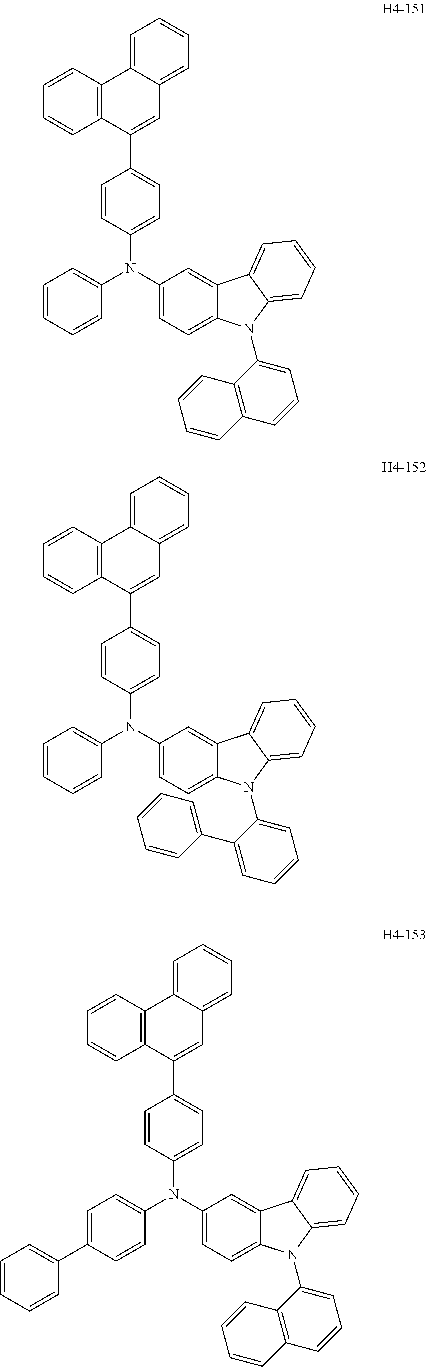 [SMILES: c1ccc(N(c2ccc(-c3cc4ccccc4c4ccccc34)cc2)c2ccc3c(c2)c2ccccc2n3-c2cccc3ccccc23)cc1 ; c1ccc(-c2ccccc2-n2c3ccccc3c3cc(N(c4ccccc4)c4ccc(-c5cc6ccccc6c6ccccc56)cc4)ccc32)cc1 ; c1ccc(-c2ccc(N(c3ccc(-c4cc5ccccc5c5ccccc45)cc3)c3ccc4c(c3)c3ccccc3n4-c3cccc4ccccc34)cc2)cc1]

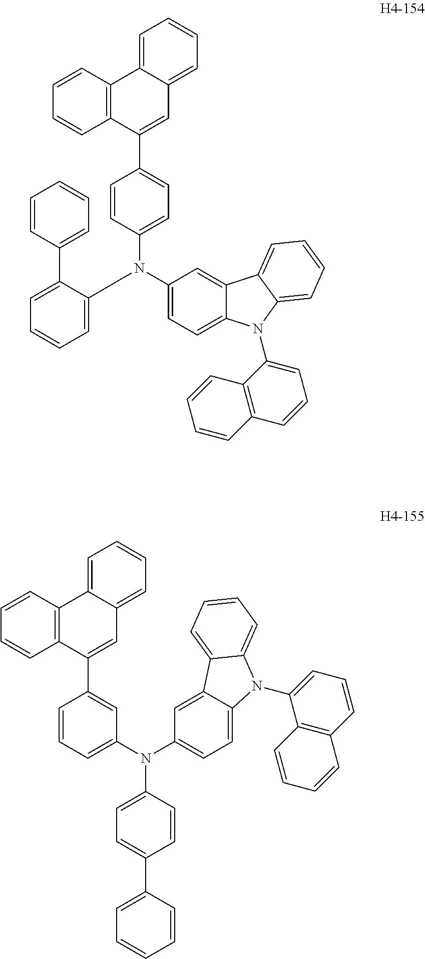 [SMILES: c1ccc(-c2ccccc2N(c2ccc(-c3cc4ccccc4c4ccccc34)cc2)c2ccc3c(c2)c2ccccc2n3-c2cccc3ccccc23)cc1 ; c1ccc(-c2ccc(N(c3cccc(-c4cc5ccccc5c5ccccc45)c3)c3ccc4c(c3)c3ccccc3n4-c3cccc4ccccc34)cc2)cc1]

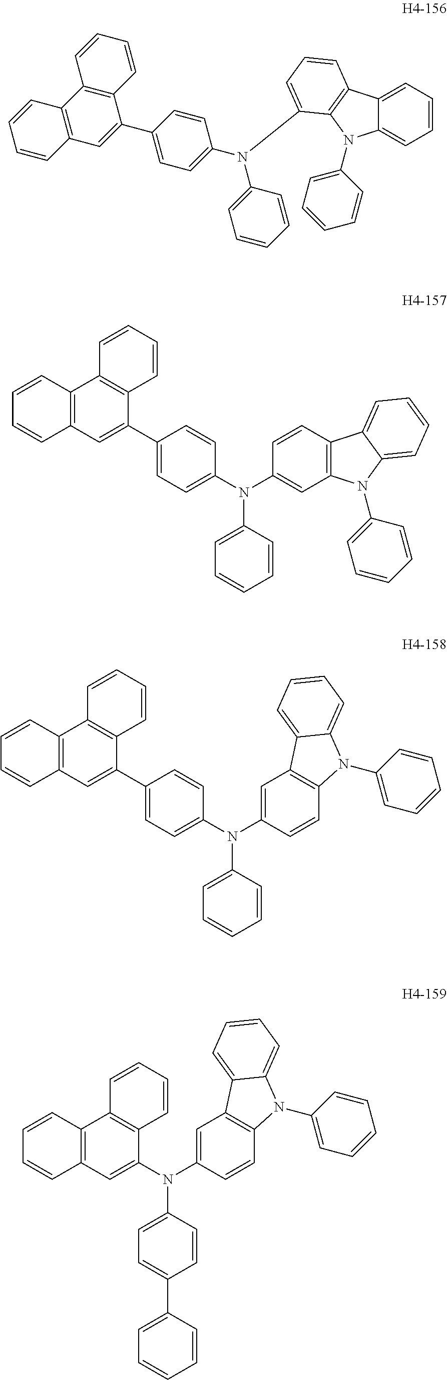 [SMILES: c1ccc(N(c2ccc(-c3cc4ccccc4c4ccccc34)cc2)c2cccc3c4ccccc4n(-c4ccccc4)c23)cc1 ; c1ccc(N(c2ccc(-c3cc4ccccc4c4ccccc34)cc2)c2ccc3c4ccccc4n(-c4ccccc4)c3c2)cc1 ; c1ccc(N(c2ccc(-c3cc4ccccc4c4ccccc34)cc2)c2ccc3c(c2)c2ccccc2n3-c2ccccc2)cc1 ; c1ccc(-c2ccc(N(c3ccc4c(c3)c3ccccc3n4-c3ccccc3)c3cc4ccccc4c4ccccc34)cc2)cc1]

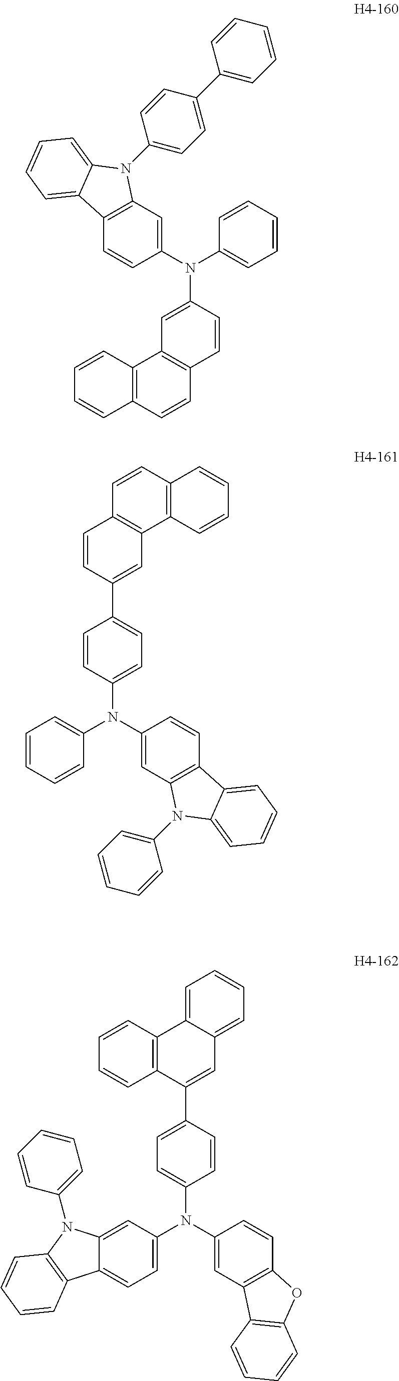 [SMILES: c1ccc(-c2ccc(-n3c4ccccc4c4ccc(N(c5ccccc5)c5ccc6ccc7ccccc7c6c5)cc43)cc2)cc1 ; c1ccc(N(c2ccc(-c3ccc4ccc5ccccc5c4c3)cc2)c2ccc3c4ccccc4n(-c4ccccc4)c3c2)cc1 ; c1ccc(-n2c3ccccc3c3ccc(N(c4ccc(-c5cc6ccccc6c6ccccc56)cc4)c4ccc5oc6ccccc6c5c4)cc32)cc1]

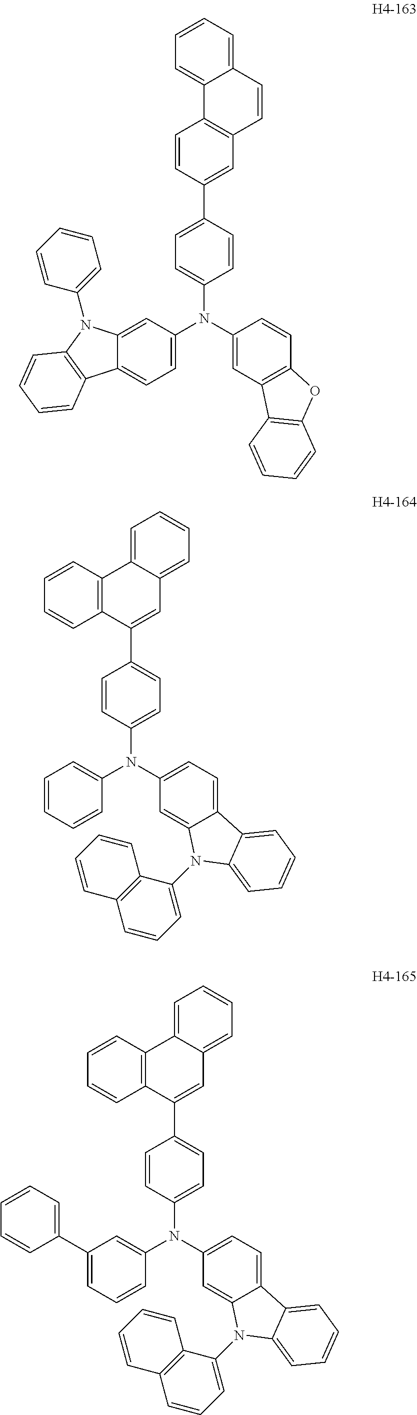 [SMILES: c1ccc(-n2c3ccccc3c3ccc(N(c4ccc(-c5ccc6c(ccc7ccccc76)c5)cc4)c4ccc5oc6ccccc6c5c4)cc32)cc1 ; c1ccc(N(c2ccc(-c3cc4ccccc4c4ccccc34)cc2)c2ccc3c4ccccc4n(-c4cccc5ccccc45)c3c2)cc1 ; c1ccc(-c2cccc(N(c3ccc(-c4cc5ccccc5c5ccccc45)cc3)c3ccc4c5ccccc5n(-c5cccc6ccccc56)c4c3)c2)cc1]

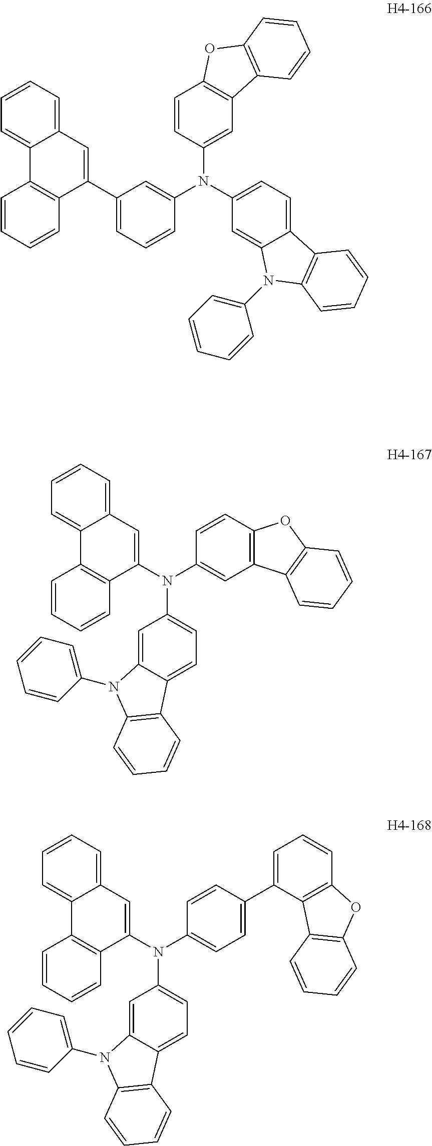 [SMILES: c1ccc(-n2c3ccccc3c3ccc(N(c4cccc(-c5cc6ccccc6c6ccccc56)c4)c4ccc5oc6ccccc6c5c4)cc32)cc1 ; c1ccc(-n2c3ccccc3c3ccc(N(c4ccc5oc6ccccc6c5c4)c4cc5ccccc5c5ccccc45)cc32)cc1 ; c1ccc(-n2c3ccccc3c3ccc(N(c4ccc(-c5cccc6oc7ccccc7c56)cc4)c4cc5ccccc5c5ccccc45)cc32)cc1]

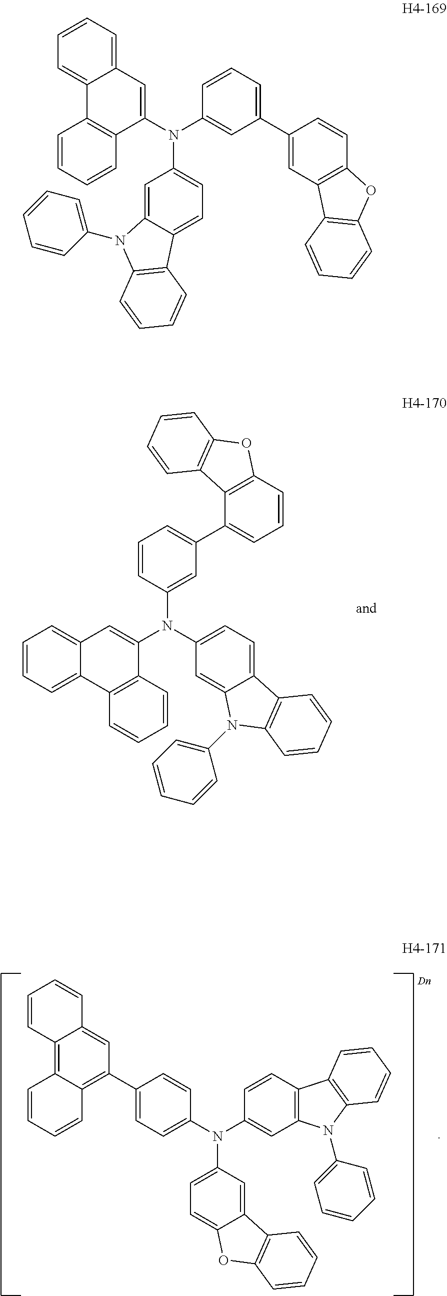

12. The plurality of host materials according to claim 8, wherein the compound represented by Formula 3 is selected from the following compounds:

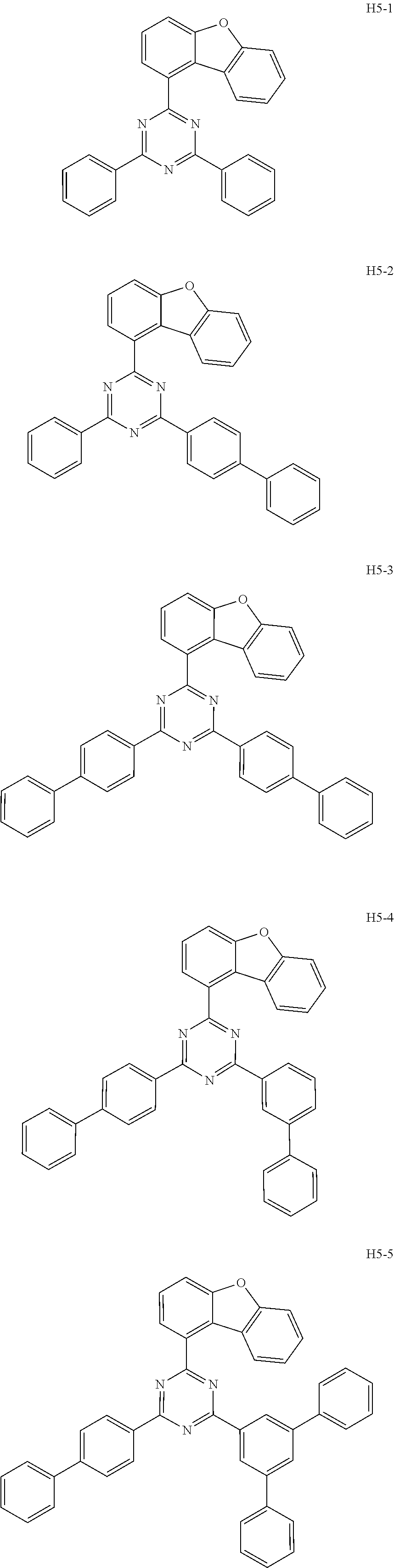

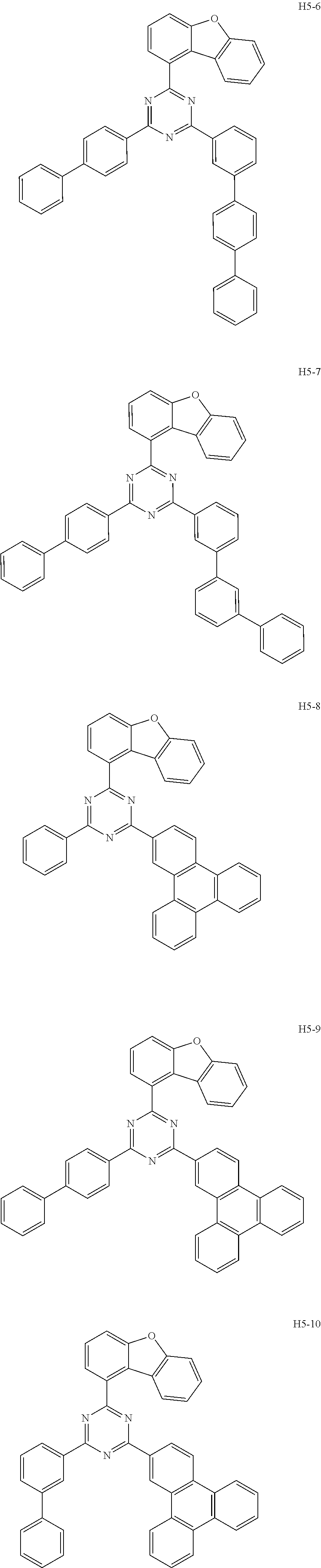

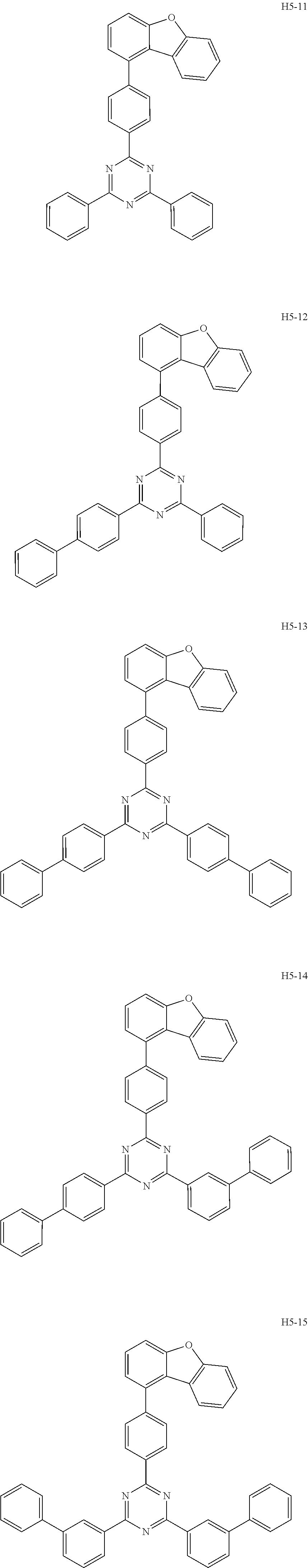

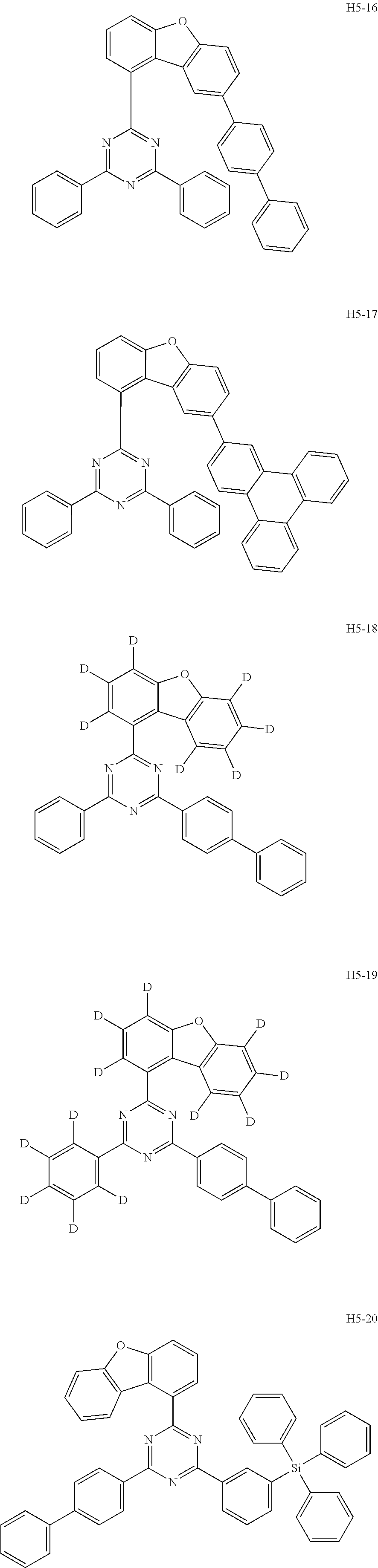

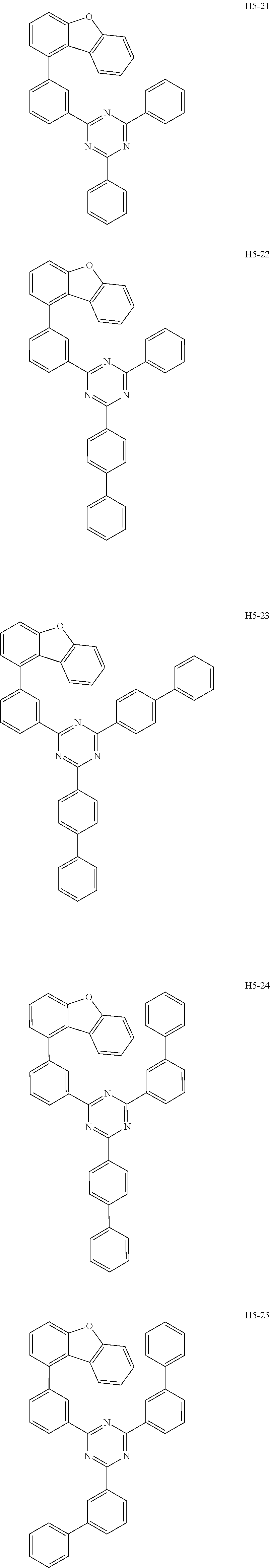

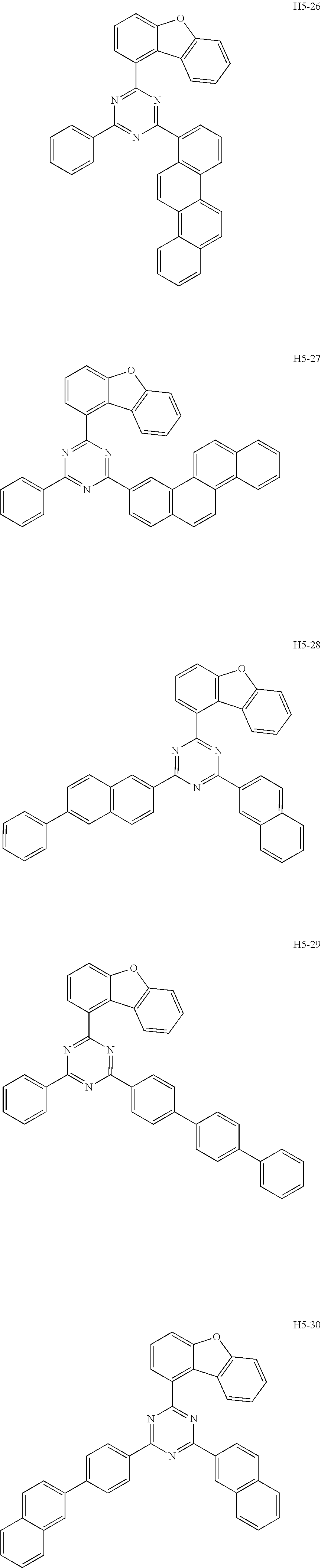

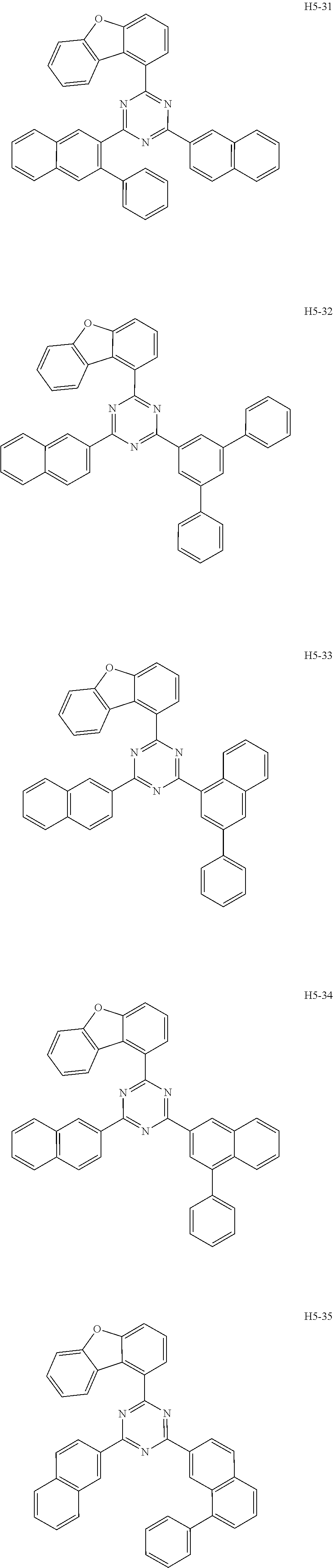 [SMILES: c1ccc(-c2cc3ccccc3cc2-c2nc(-c3ccc4ccccc4c3)nc(-c3cccc4oc5ccccc5c34)n2)cc1 ; c1ccc(-c2cc(-c3ccccc3)cc(-c3nc(-c4ccc5ccccc5c4)nc(-c4cccc5oc6ccccc6c45)n3)c2)cc1 ; c1ccc(-c2cc(-c3nc(-c4ccc5ccccc5c4)nc(-c4cccc5oc6ccccc6c45)n3)c3ccccc3c2)cc1 ; c1ccc(-c2cc(-c3nc(-c4ccc5ccccc5c4)nc(-c4cccc5oc6ccccc6c45)n3)cc3ccccc23)cc1 ; c1ccc(-c2cccc3ccc(-c4nc(-c5ccc6ccccc6c5)nc(-c5cccc6oc7ccccc7c56)n4)cc23)cc1]

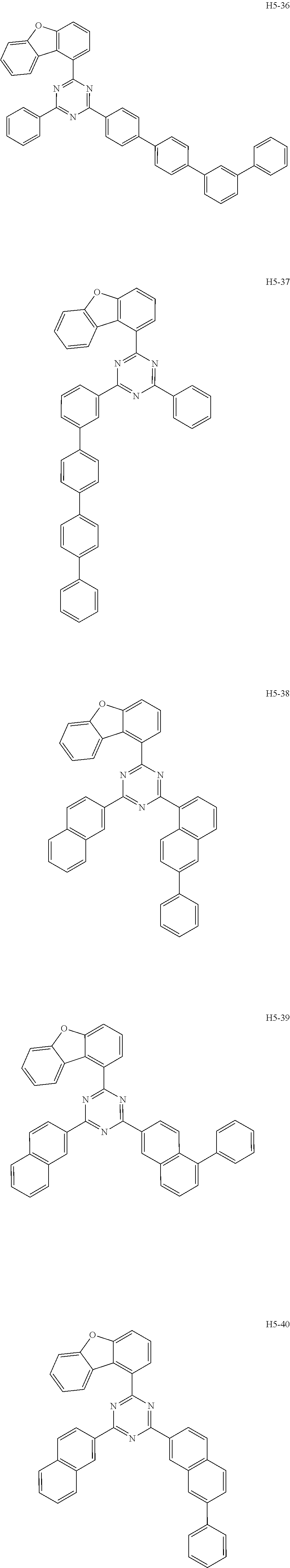 [SMILES: c1ccc(-c2cccc(-c3ccc(-c4ccc(-c5nc(-c6ccccc6)nc(-c6cccc7oc8ccccc8c67)n5)cc4)cc3)c2)cc1 ; c1ccc(-c2ccc(-c3ccc(-c4cccc(-c5nc(-c6ccccc6)nc(-c6cccc7oc8ccccc8c67)n5)c4)cc3)cc2)cc1 ; c1ccc(-c2ccc3c(-c4nc(-c5ccc6ccccc6c5)nc(-c5cccc6oc7ccccc7c56)n4)cccc3c2)cc1 ; c1ccc(-c2cccc3cc(-c4nc(-c5ccc6ccccc6c5)nc(-c5cccc6oc7ccccc7c56)n4)ccc23)cc1 ; c1ccc(-c2ccc3ccc(-c4nc(-c5ccc6ccccc6c5)nc(-c5cccc6oc7ccccc7c56)n4)cc3c2)cc1]

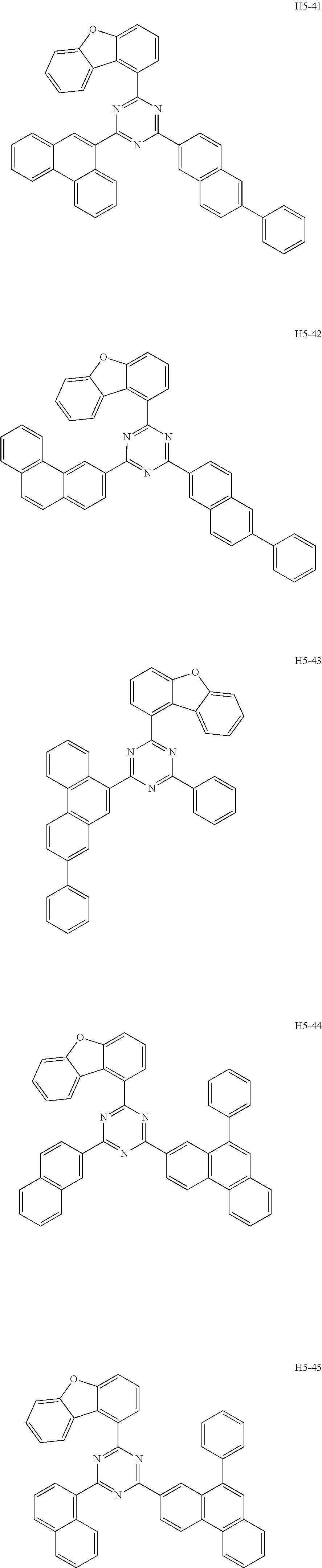 [SMILES: c1ccc(-c2ccc3cc(-c4nc(-c5cc6ccccc6c6ccccc56)nc(-c5cccc6oc7ccccc7c56)n4)ccc3c2)cc1 ; c1ccc(-c2ccc3cc(-c4nc(-c5ccc6ccc7ccccc7c6c5)nc(-c5cccc6oc7ccccc7c56)n4)ccc3c2)cc1 ; c1ccc(-c2ccc3c(c2)cc(-c2nc(-c4ccccc4)nc(-c4cccc5oc6ccccc6c45)n2)c2ccccc23)cc1 ; c1ccc(-c2cc3ccccc3c3ccc(-c4nc(-c5ccc6ccccc6c5)nc(-c5cccc6oc7ccccc7c56)n4)cc23)cc1 ; c1ccc(-c2cc3ccccc3c3ccc(-c4nc(-c5cccc6ccccc56)nc(-c5cccc6oc7ccccc7c56)n4)cc23)cc1]

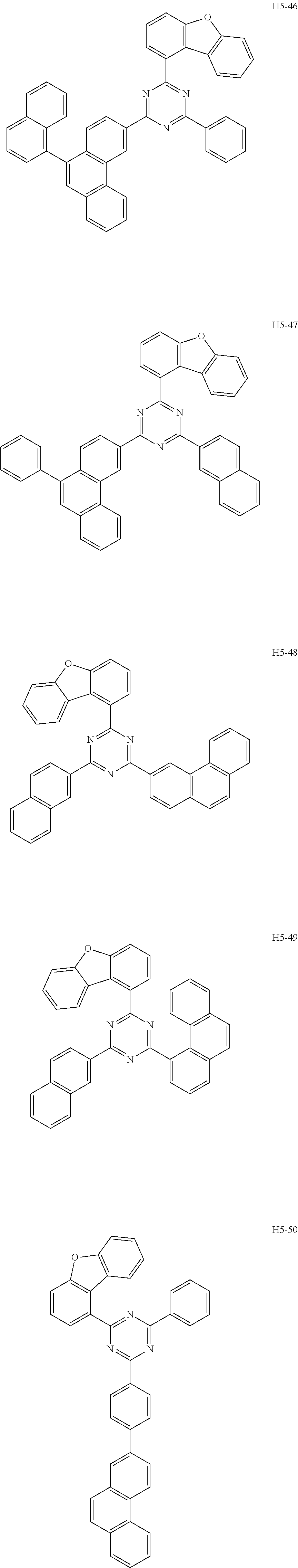 [SMILES: c1ccc(-c2nc(-c3ccc4c(-c5cccc6ccccc56)cc5ccccc5c4c3)nc(-c3cccc4oc5ccccc5c34)n2)cc1 ; c1ccc(-c2cc3ccccc3c3cc(-c4nc(-c5ccc6ccccc6c5)nc(-c5cccc6oc7ccccc7c56)n4)ccc23)cc1 ; c1ccc2cc(-c3nc(-c4ccc5ccc6ccccc6c5c4)nc(-c4cccc5oc6ccccc6c45)n3)ccc2c1 ; c1ccc2cc(-c3nc(-c4cccc5ccc6ccccc6c45)nc(-c4cccc5oc6ccccc6c45)n3)ccc2c1 ; c1ccc(-c2nc(-c3ccc(-c4ccc5c(ccc6ccccc65)c4)cc3)nc(-c3cccc4oc5ccccc5c34)n2)cc1]

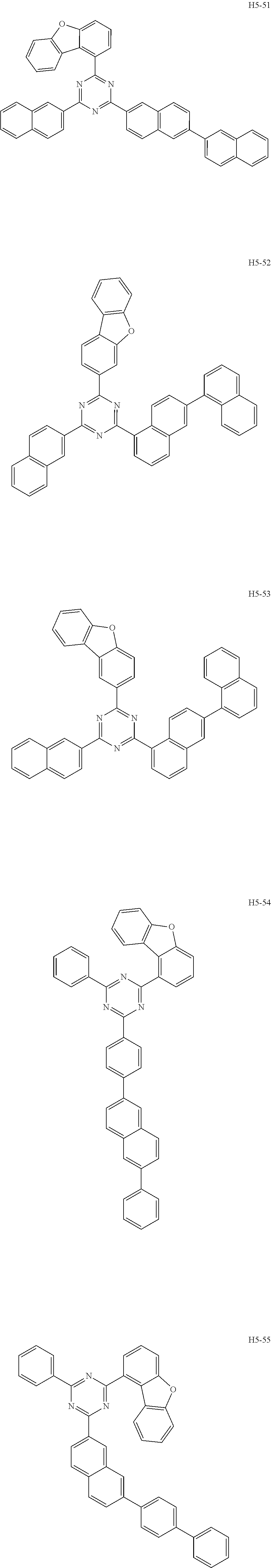

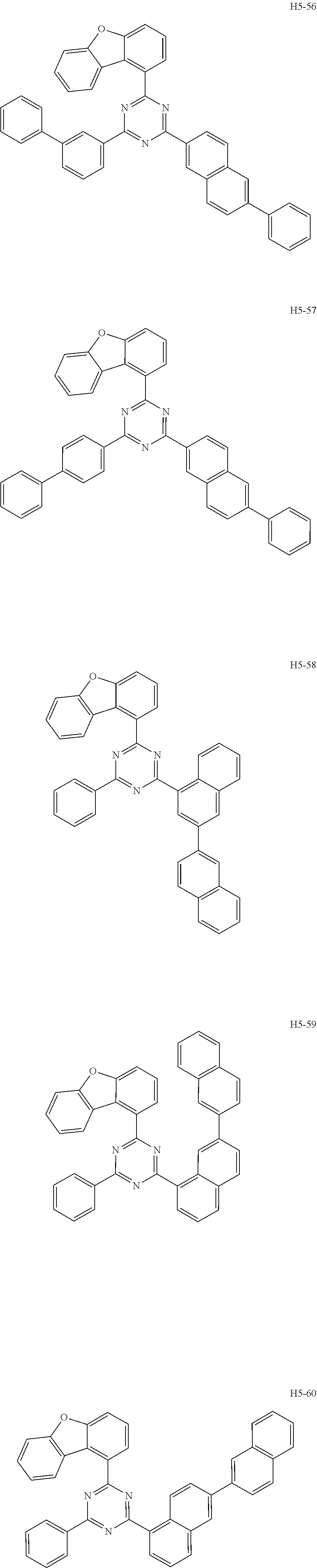 [SMILES: c1ccc(-c2cccc(-c3nc(-c4ccc5cc(-c6ccccc6)ccc5c4)nc(-c4cccc5oc6ccccc6c45)n3)c2)cc1 ; c1ccc(-c2ccc(-c3nc(-c4ccc5cc(-c6ccccc6)ccc5c4)nc(-c4cccc5oc6ccccc6c45)n3)cc2)cc1 ; c1ccc(-c2nc(-c3cc(-c4ccc5ccccc5c4)cc4ccccc34)nc(-c3cccc4oc5ccccc5c34)n2)cc1 ; c1ccc(-c2nc(-c3cccc4ccc(-c5ccc6ccccc6c5)cc34)nc(-c3cccc4oc5ccccc5c34)n2)cc1 ; c1ccc(-c2nc(-c3cccc4cc(-c5ccc6ccccc6c5)ccc34)nc(-c3cccc4oc5ccccc5c34)n2)cc1]

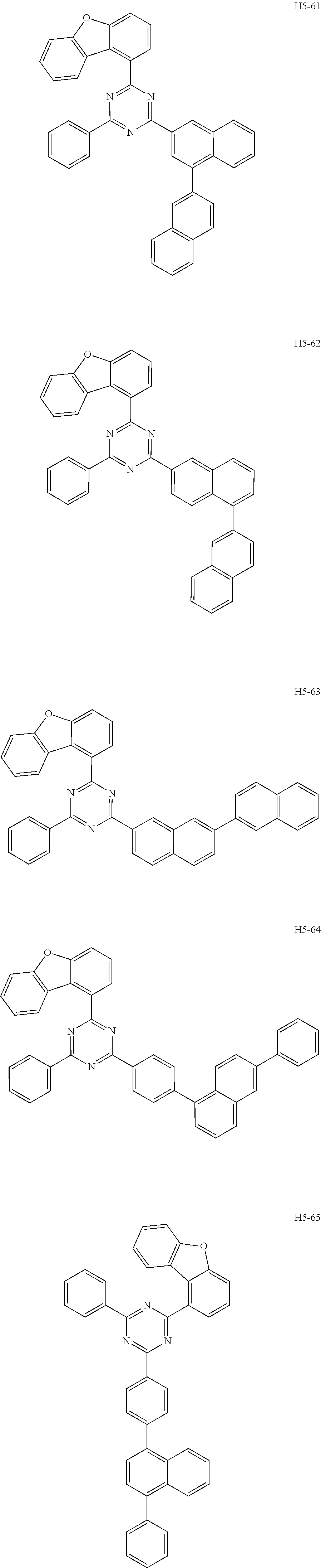 [SMILES: c1ccc(-c2nc(-c3cc(-c4ccc5ccccc5c4)c4ccccc4c3)nc(-c3cccc4oc5ccccc5c34)n2)cc1 ; c1ccc(-c2nc(-c3ccc4c(-c5ccc6ccccc6c5)cccc4c3)nc(-c3cccc4oc5ccccc5c34)n2)cc1 ; c1ccc(-c2nc(-c3ccc4ccc(-c5ccc6ccccc6c5)cc4c3)nc(-c3cccc4oc5ccccc5c34)n2)cc1 ; c1ccc(-c2ccc3c(-c4ccc(-c5nc(-c6ccccc6)nc(-c6cccc7oc8ccccc8c67)n5)cc4)cccc3c2)cc1 ; c1ccc(-c2nc(-c3ccc(-c4ccc(-c5ccccc5)c5ccccc45)cc3)nc(-c3cccc4oc5ccccc5c34)n2)cc1]

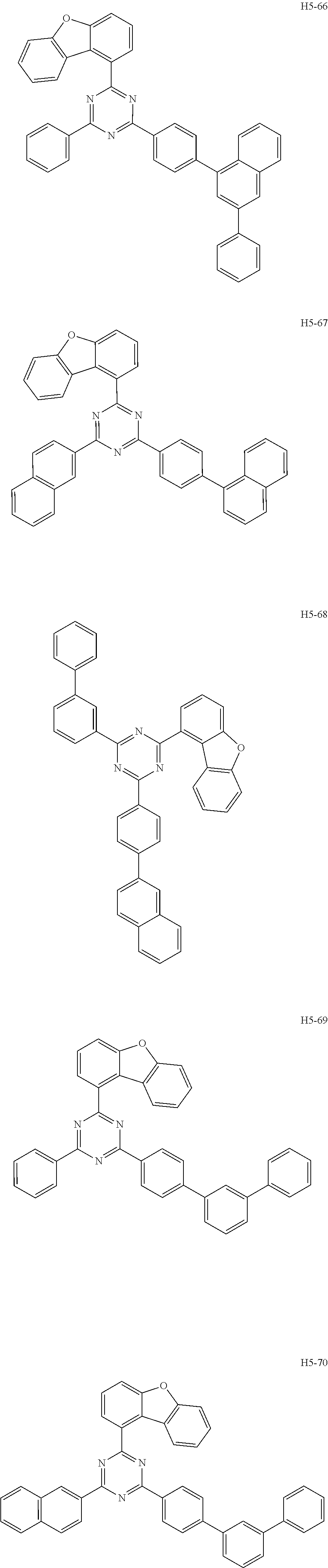 [SMILES: c1ccc(-c2cc(-c3ccc(-c4nc(-c5ccccc5)nc(-c5cccc6oc7ccccc7c56)n4)cc3)c3ccccc3c2)cc1 ; c1ccc2cc(-c3nc(-c4ccc(-c5cccc6ccccc56)cc4)nc(-c4cccc5oc6ccccc6c45)n3)ccc2c1 ; c1ccc(-c2cccc(-c3nc(-c4ccc(-c5ccc6ccccc6c5)cc4)nc(-c4cccc5oc6ccccc6c45)n3)c2)cc1 ; c1ccc(-c2cccc(-c3ccc(-c4nc(-c5ccccc5)nc(-c5cccc6oc7ccccc7c56)n4)cc3)c2)cc1 ; c1ccc(-c2cccc(-c3ccc(-c4nc(-c5ccc6ccccc6c5)nc(-c5cccc6oc7ccccc7c56)n4)cc3)c2)cc1]

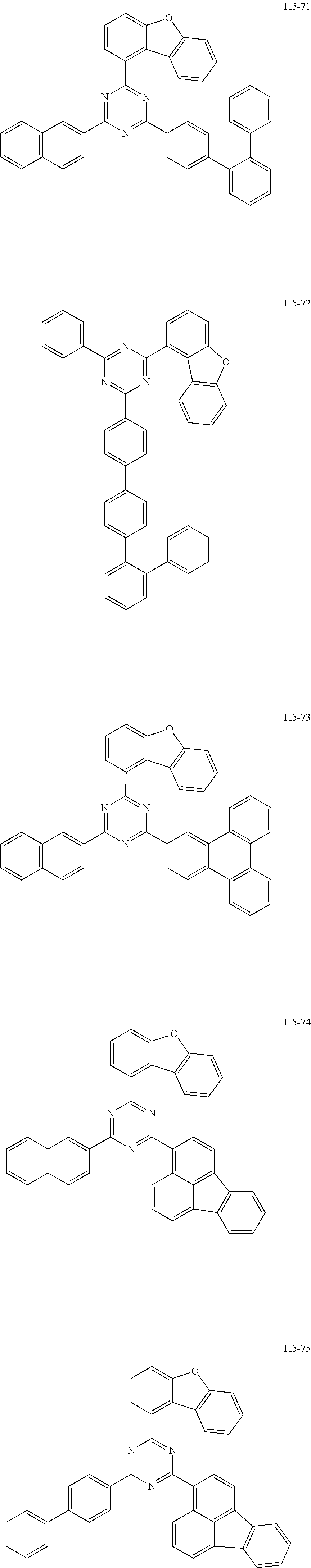 [SMILES: c1ccc(-c2ccccc2-c2ccc(-c3nc(-c4ccc5ccccc5c4)nc(-c4cccc5oc6ccccc6c45)n3)cc2)cc1 ; c1ccc(-c2nc(-c3ccc(-c4ccc(-c5ccccc5-c5ccccc5)cc4)cc3)nc(-c3cccc4oc5ccccc5c34)n2)cc1 ; c1ccc2cc(-c3nc(-c4ccc5c6ccccc6c6ccccc6c5c4)nc(-c4cccc5oc6ccccc6c45)n3)ccc2c1 ; c1ccc2c(c1)-c1cccc3c(-c4nc(-c5ccc6ccccc6c5)nc(-c5cccc6oc7ccccc7c56)n4)ccc-2c13 ; c1ccc(-c2ccc(-c3nc(-c4ccc5c6c(cccc46)-c4ccccc4-5)nc(-c4cccc5oc6ccccc6c45)n3)cc2)cc1]

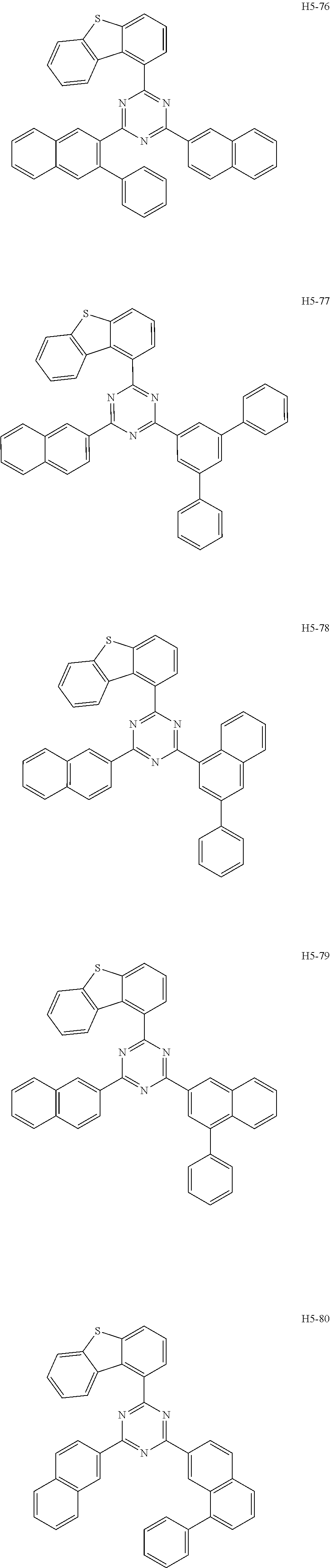 [SMILES: c1ccc(-c2cc3ccccc3cc2-c2nc(-c3ccc4ccccc4c3)nc(-c3cccc4sc5ccccc5c34)n2)cc1 ; c1ccc(-c2cc(-c3ccccc3)cc(-c3nc(-c4ccc5ccccc5c4)nc(-c4cccc5sc6ccccc6c45)n3)c2)cc1 ; c1ccc(-c2cc(-c3nc(-c4ccc5ccccc5c4)nc(-c4cccc5sc6ccccc6c45)n3)c3ccccc3c2)cc1 ; c1ccc(-c2cc(-c3nc(-c4ccc5ccccc5c4)nc(-c4cccc5sc6ccccc6c45)n3)cc3ccccc23)cc1 ; c1ccc(-c2cccc3ccc(-c4nc(-c5ccc6ccccc6c5)nc(-c5cccc6sc7ccccc7c56)n4)cc23)cc1]

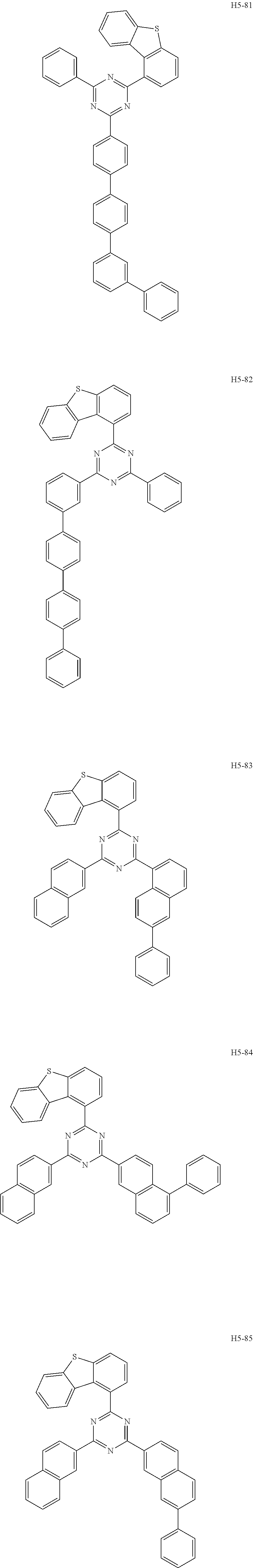 [SMILES: c1ccc(-c2cccc(-c3ccc(-c4ccc(-c5nc(-c6ccccc6)nc(-c6cccc7sc8ccccc8c67)n5)cc4)cc3)c2)cc1 ; c1ccc(-c2ccc(-c3ccc(-c4cccc(-c5nc(-c6ccccc6)nc(-c6cccc7sc8ccccc8c67)n5)c4)cc3)cc2)cc1 ; c1ccc(-c2ccc3c(-c4nc(-c5ccc6ccccc6c5)nc(-c5cccc6sc7ccccc7c56)n4)cccc3c2)cc1 ; c1ccc(-c2cccc3cc(-c4nc(-c5ccc6ccccc6c5)nc(-c5cccc6sc7ccccc7c56)n4)ccc23)cc1 ; c1ccc(-c2ccc3ccc(-c4nc(-c5ccc6ccccc6c5)nc(-c5cccc6sc7ccccc7c56)n4)cc3c2)cc1]

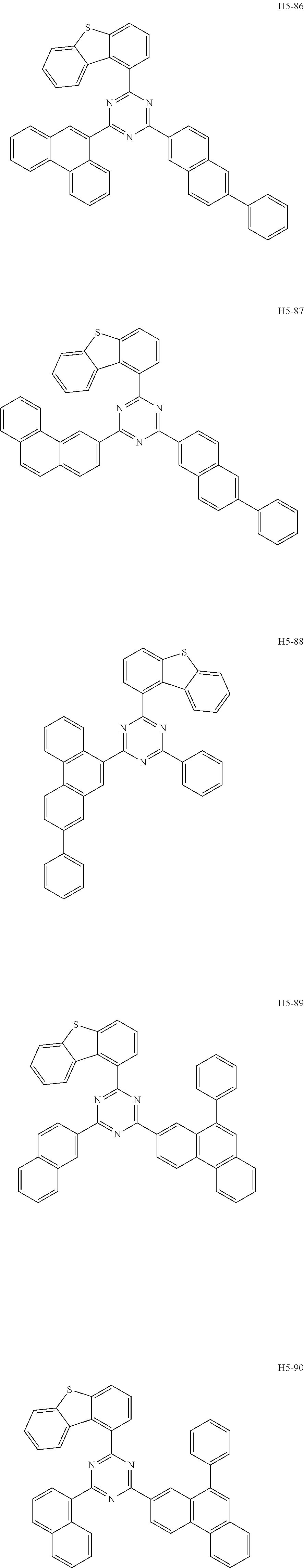 [SMILES: c1ccc(-c2ccc3cc(-c4nc(-c5cc6ccccc6c6ccccc56)nc(-c5cccc6sc7ccccc7c56)n4)ccc3c2)cc1 ; c1ccc(-c2ccc3cc(-c4nc(-c5ccc6ccc7ccccc7c6c5)nc(-c5cccc6sc7ccccc7c56)n4)ccc3c2)cc1 ; c1ccc(-c2ccc3c(c2)cc(-c2nc(-c4ccccc4)nc(-c4cccc5sc6ccccc6c45)n2)c2ccccc23)cc1 ; c1ccc(-c2cc3ccccc3c3ccc(-c4nc(-c5ccc6ccccc6c5)nc(-c5cccc6sc7ccccc7c56)n4)cc23)cc1 ; c1ccc(-c2cc3ccccc3c3ccc(-c4nc(-c5cccc6ccccc56)nc(-c5cccc6sc7ccccc7c56)n4)cc23)cc1]

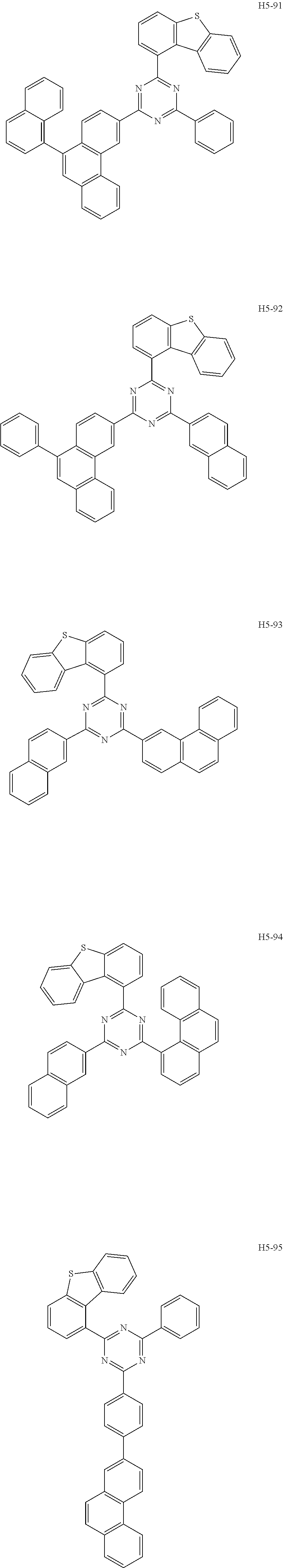 [SMILES: c1ccc(-c2nc(-c3ccc4c(-c5cccc6ccccc56)cc5ccccc5c4c3)nc(-c3cccc4sc5ccccc5c34)n2)cc1 ; c1ccc(-c2cc3ccccc3c3cc(-c4nc(-c5ccc6ccccc6c5)nc(-c5cccc6sc7ccccc7c56)n4)ccc23)cc1 ; c1ccc2cc(-c3nc(-c4ccc5ccc6ccccc6c5c4)nc(-c4cccc5sc6ccccc6c45)n3)ccc2c1 ; c1ccc2cc(-c3nc(-c4cccc5ccc6ccccc6c45)nc(-c4cccc5sc6ccccc6c45)n3)ccc2c1 ; c1ccc(-c2nc(-c3ccc(-c4ccc5c(ccc6ccccc65)c4)cc3)nc(-c3cccc4sc5ccccc5c34)n2)cc1]

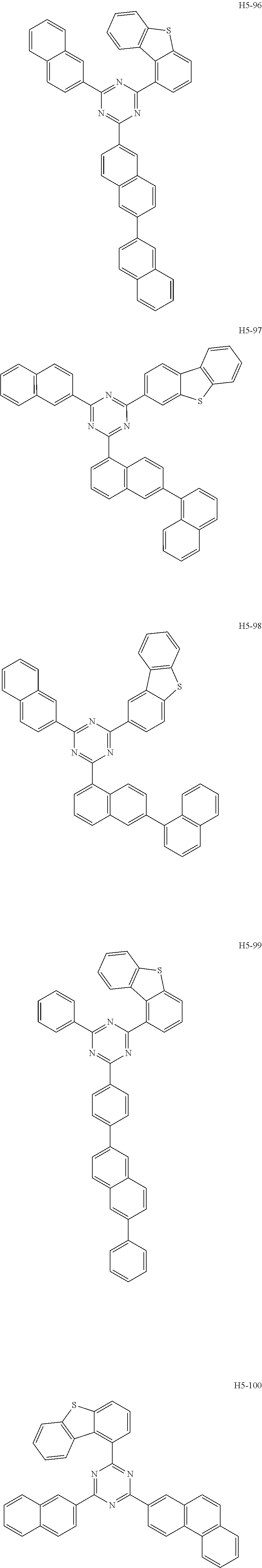 [SMILES: c1ccc2cc(-c3ccc4cc(-c5nc(-c6ccc7ccccc7c6)nc(-c6cccc7sc8ccccc8c67)n5)ccc4c3)ccc2c1 ; c1ccc2cc(-c3nc(-c4ccc5c(c4)sc4ccccc45)nc(-c4cccc5cc(-c6cccc7ccccc67)ccc45)n3)ccc2c1 ; c1ccc2cc(-c3nc(-c4ccc5sc6ccccc6c5c4)nc(-c4cccc5cc(-c6cccc7ccccc67)ccc45)n3)ccc2c1 ; c1ccc(-c2ccc3cc(-c4ccc(-c5nc(-c6ccccc6)nc(-c6cccc7sc8ccccc8c67)n5)cc4)ccc3c2)cc1 ; c1ccc2cc(-c3nc(-c4ccc5c(ccc6ccccc65)c4)nc(-c4cccc5sc6ccccc6c45)n3)ccc2c1]

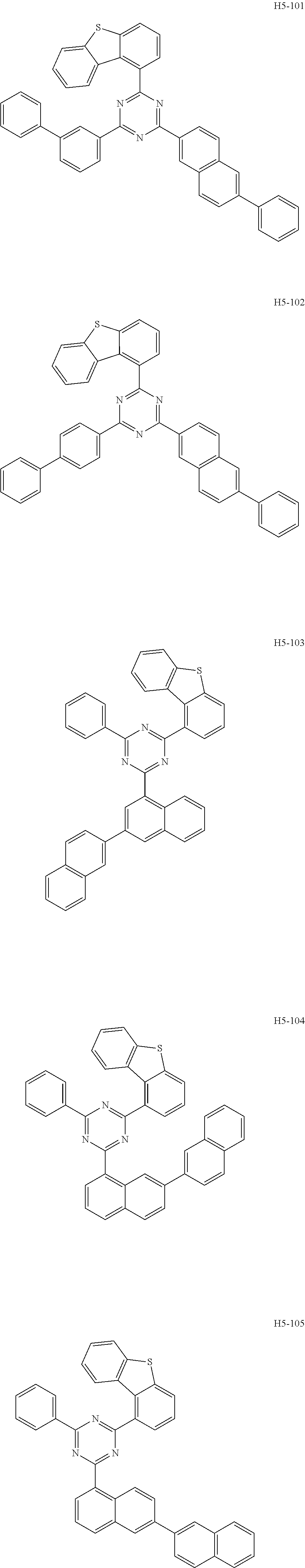 [SMILES: c1ccc(-c2cccc(-c3nc(-c4ccc5cc(-c6ccccc6)ccc5c4)nc(-c4cccc5sc6ccccc6c45)n3)c2)cc1 ; c1ccc(-c2ccc(-c3nc(-c4ccc5cc(-c6ccccc6)ccc5c4)nc(-c4cccc5sc6ccccc6c45)n3)cc2)cc1 ; c1ccc(-c2nc(-c3cc(-c4ccc5ccccc5c4)cc4ccccc34)nc(-c3cccc4sc5ccccc5c34)n2)cc1 ; c1ccc(-c2nc(-c3cccc4ccc(-c5ccc6ccccc6c5)cc34)nc(-c3cccc4sc5ccccc5c34)n2)cc1 ; c1ccc(-c2nc(-c3cccc4cc(-c5ccc6ccccc6c5)ccc34)nc(-c3cccc4sc5ccccc5c34)n2)cc1]

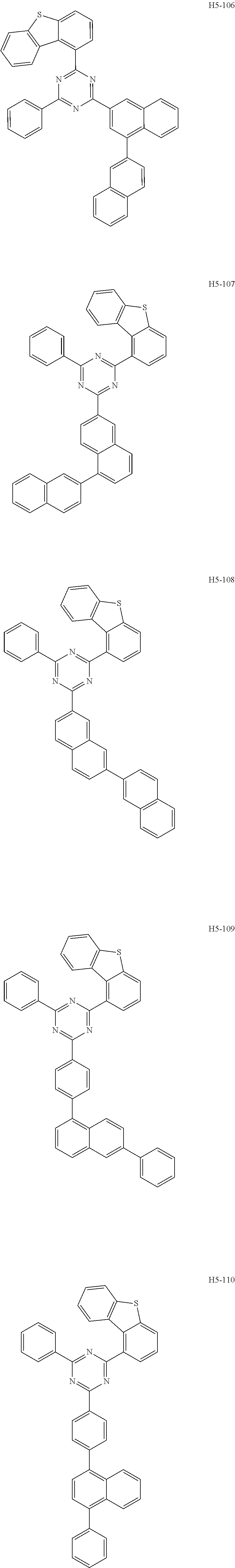 [SMILES: c1ccc(-c2nc(-c3cc(-c4ccc5ccccc5c4)c4ccccc4c3)nc(-c3cccc4sc5ccccc5c34)n2)cc1 ; c1ccc(-c2nc(-c3ccc4c(-c5ccc6ccccc6c5)cccc4c3)nc(-c3cccc4sc5ccccc5c34)n2)cc1 ; c1ccc(-c2nc(-c3ccc4ccc(-c5ccc6ccccc6c5)cc4c3)nc(-c3cccc4sc5ccccc5c34)n2)cc1 ; c1ccc(-c2ccc3c(-c4ccc(-c5nc(-c6ccccc6)nc(-c6cccc7sc8ccccc8c67)n5)cc4)cccc3c2)cc1 ; c1ccc(-c2nc(-c3ccc(-c4ccc(-c5ccccc5)c5ccccc45)cc3)nc(-c3cccc4sc5ccccc5c34)n2)cc1]

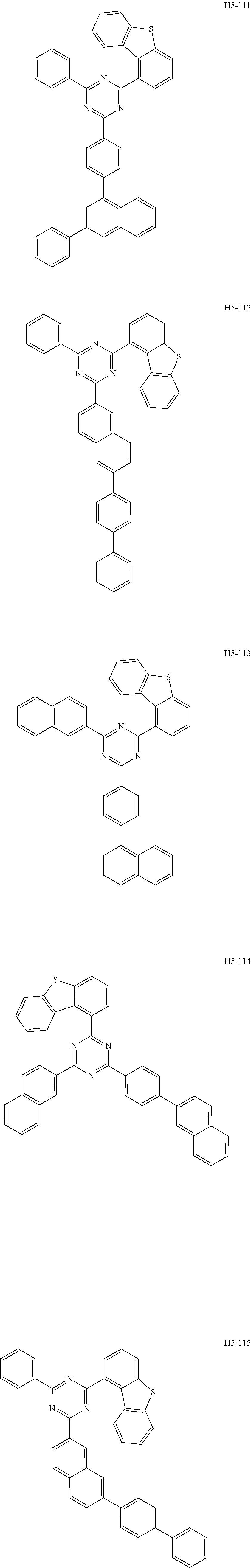

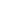

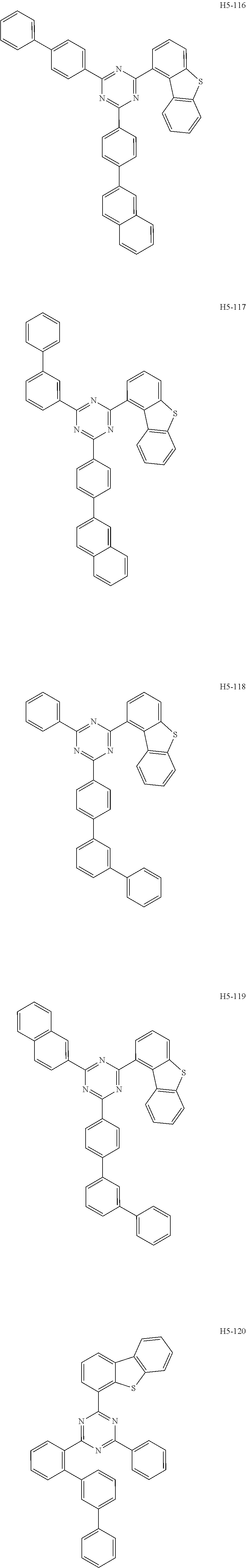 [SMILES: c1ccc(-c2ccc(-c3nc(-c4ccc(-c5ccc6ccccc6c5)cc4)nc(-c4cccc5sc6ccccc6c45)n3)cc2)cc1 ; c1ccc(-c2cccc(-c3nc(-c4ccc(-c5ccc6ccccc6c5)cc4)nc(-c4cccc5sc6ccccc6c45)n3)c2)cc1 ; c1ccc(-c2cccc(-c3ccc(-c4nc(-c5ccccc5)nc(-c5cccc6sc7ccccc7c56)n4)cc3)c2)cc1 ; c1ccc(-c2cccc(-c3ccc(-c4nc(-c5ccc6ccccc6c5)nc(-c5cccc6sc7ccccc7c56)n4)cc3)c2)cc1 ; c1ccc(-c2cccc(-c3ccccc3-c3nc(-c4ccccc4)nc(-c4cccc5c4sc4ccccc45)n3)c2)cc1]

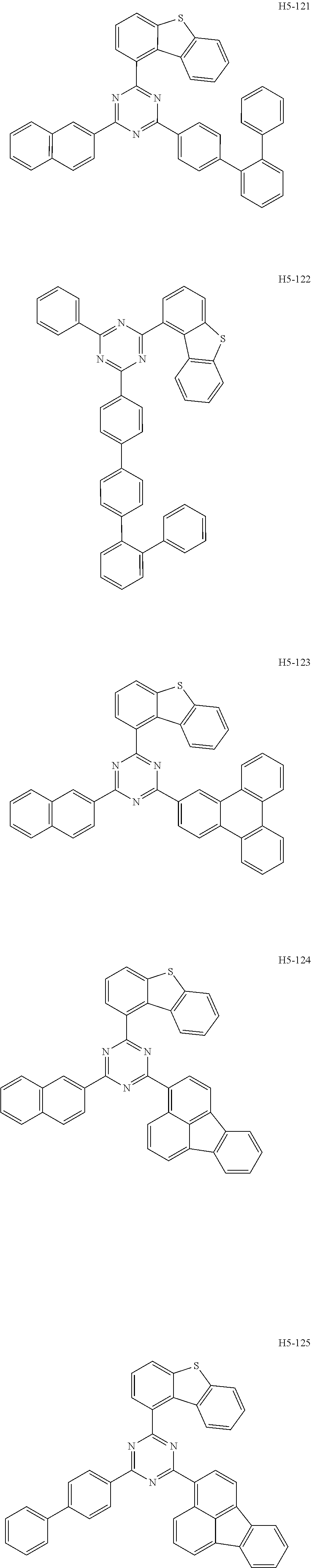 [SMILES: c1ccc(-c2ccccc2-c2ccc(-c3nc(-c4ccc5ccccc5c4)nc(-c4cccc5sc6ccccc6c45)n3)cc2)cc1 ; c1ccc(-c2nc(-c3ccc(-c4ccc(-c5ccccc5-c5ccccc5)cc4)cc3)nc(-c3cccc4sc5ccccc5c34)n2)cc1 ; c1ccc2cc(-c3nc(-c4ccc5c6ccccc6c6ccccc6c5c4)nc(-c4cccc5sc6ccccc6c45)n3)ccc2c1 ; c1ccc2c(c1)-c1cccc3c(-c4nc(-c5ccc6ccccc6c5)nc(-c5cccc6sc7ccccc7c56)n4)ccc-2c13 ; c1ccc(-c2ccc(-c3nc(-c4ccc5c6c(cccc46)-c4ccccc4-5)nc(-c4cccc5sc6ccccc6c45)n3)cc2)cc1]

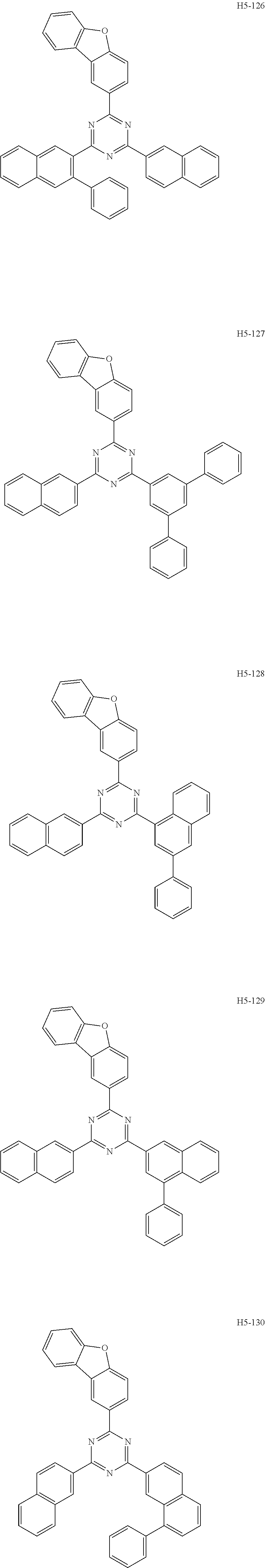 [SMILES: c1ccc(-c2cc3ccccc3cc2-c2nc(-c3ccc4ccccc4c3)nc(-c3ccc4oc5ccccc5c4c3)n2)cc1 ; c1ccc(-c2cc(-c3ccccc3)cc(-c3nc(-c4ccc5ccccc5c4)nc(-c4ccc5oc6ccccc6c5c4)n3)c2)cc1 ; c1ccc(-c2cc(-c3nc(-c4ccc5ccccc5c4)nc(-c4ccc5oc6ccccc6c5c4)n3)c3ccccc3c2)cc1 ; c1ccc(-c2cc(-c3nc(-c4ccc5ccccc5c4)nc(-c4ccc5oc6ccccc6c5c4)n3)cc3ccccc23)cc1 ; c1ccc(-c2cccc3ccc(-c4nc(-c5ccc6ccccc6c5)nc(-c5ccc6oc7ccccc7c6c5)n4)cc23)cc1]

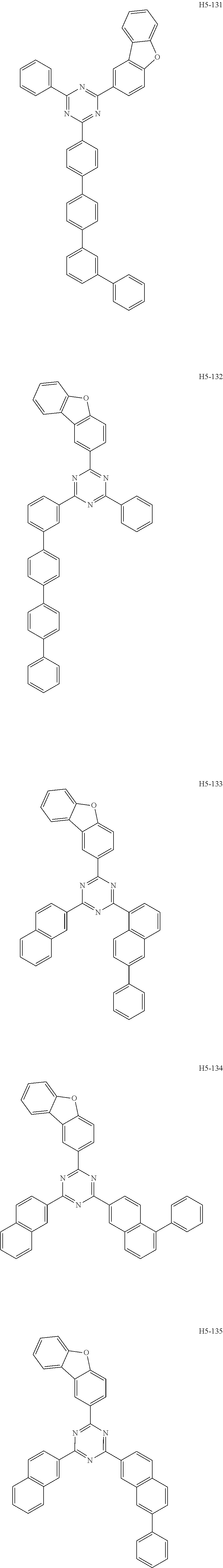

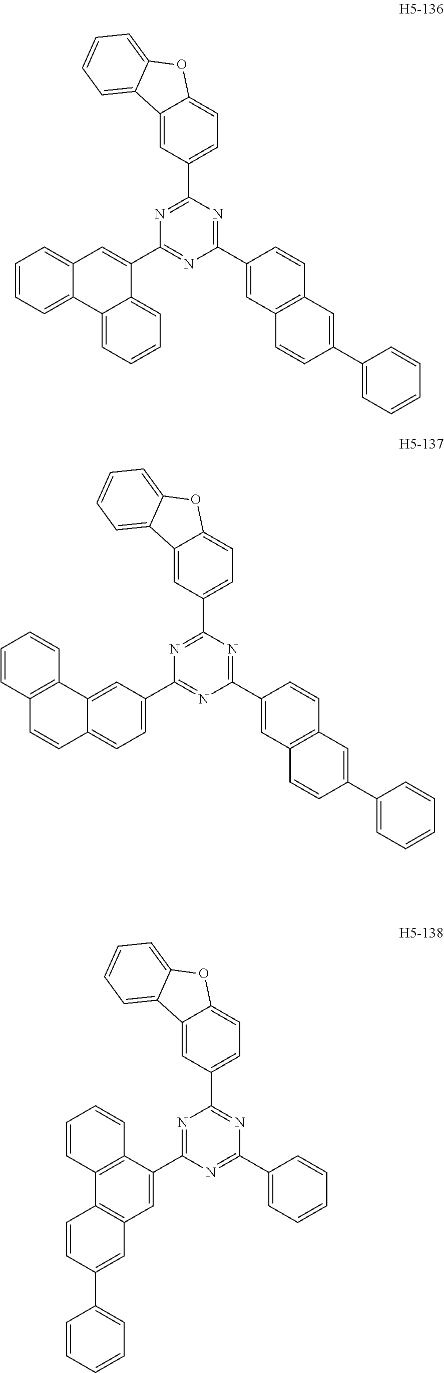 [SMILES: c1ccc(-c2ccc3cc(-c4nc(-c5ccc6oc7ccccc7c6c5)nc(-c5cc6ccccc6c6ccccc56)n4)ccc3c2)cc1 ; c1ccc(-c2ccc3cc(-c4nc(-c5ccc6ccc7ccccc7c6c5)nc(-c5ccc6oc7ccccc7c6c5)n4)ccc3c2)cc1 ; c1ccc(-c2ccc3c(c2)cc(-c2nc(-c4ccccc4)nc(-c4ccc5oc6ccccc6c5c4)n2)c2ccccc23)cc1]

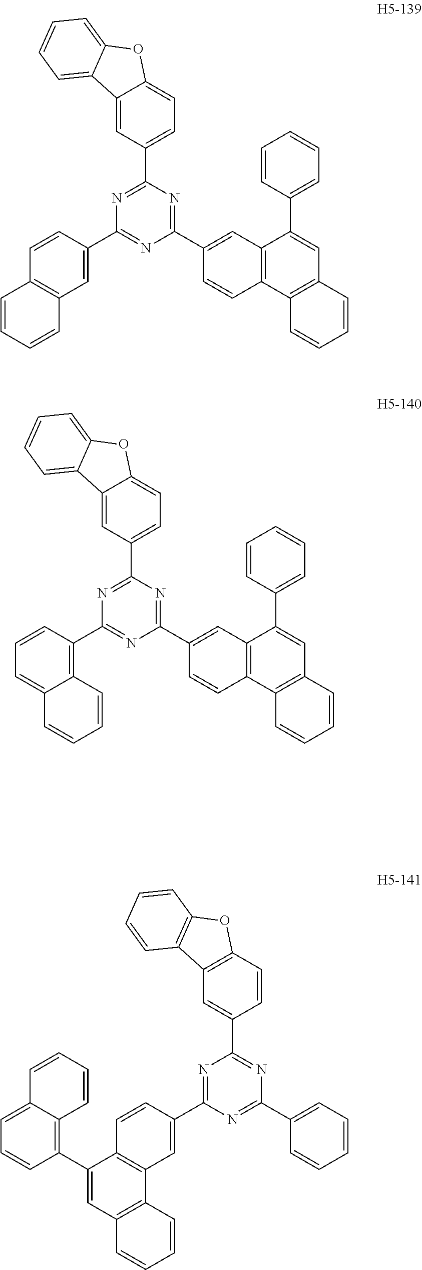 [SMILES: c1ccc(-c2cc3ccccc3c3ccc(-c4nc(-c5ccc6ccccc6c5)nc(-c5ccc6oc7ccccc7c6c5)n4)cc23)cc1 ; c1ccc(-c2cc3ccccc3c3ccc(-c4nc(-c5ccc6oc7ccccc7c6c5)nc(-c5cccc6ccccc56)n4)cc23)cc1 ; c1ccc(-c2nc(-c3ccc4oc5ccccc5c4c3)nc(-c3ccc4c(-c5cccc6ccccc56)cc5ccccc5c4c3)n2)cc1]

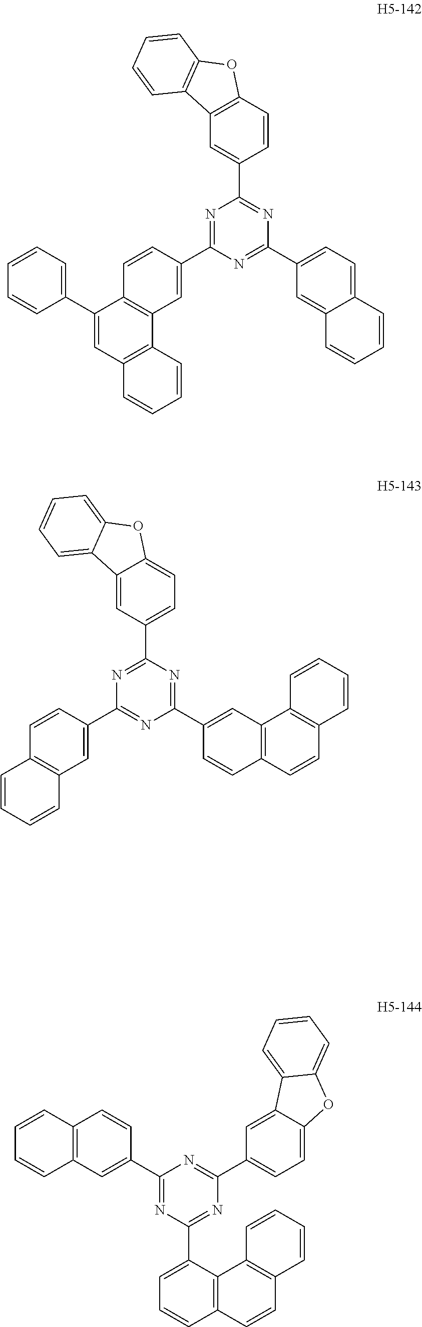 [SMILES: c1ccc(-c2cc3ccccc3c3cc(-c4nc(-c5ccc6ccccc6c5)nc(-c5ccc6oc7ccccc7c6c5)n4)ccc23)cc1 ; c1ccc2cc(-c3nc(-c4ccc5ccc6ccccc6c5c4)nc(-c4ccc5oc6ccccc6c5c4)n3)ccc2c1 ; c1ccc2cc(-c3nc(-c4ccc5oc6ccccc6c5c4)nc(-c4cccc5ccc6ccccc6c45)n3)ccc2c1]

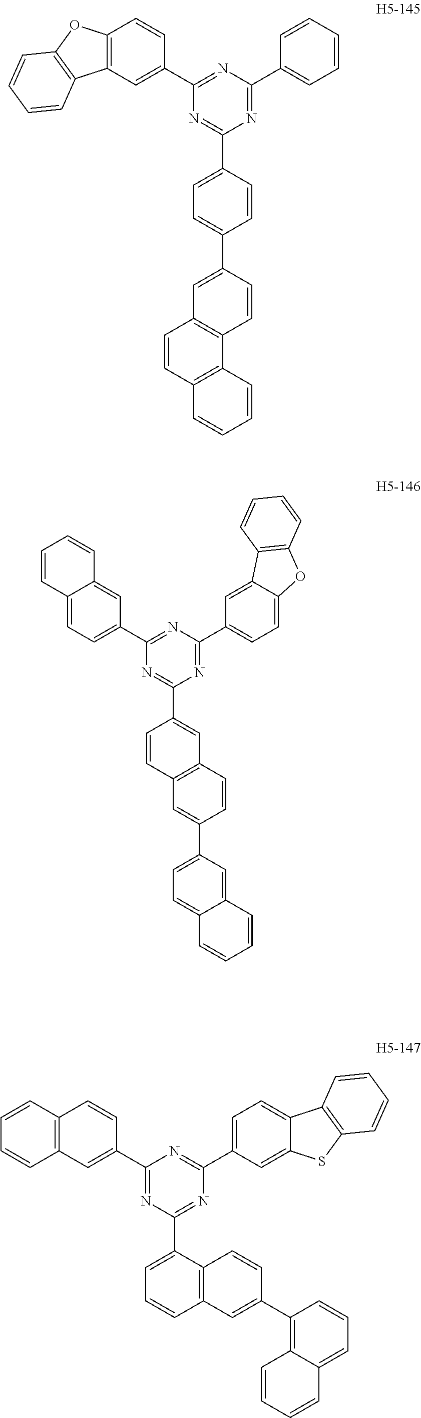 [SMILES: c1ccc(-c2nc(-c3ccc(-c4ccc5c(ccc6ccccc65)c4)cc3)nc(-c3ccc4oc5ccccc5c4c3)n2)cc1 ; c1ccc2cc(-c3ccc4cc(-c5nc(-c6ccc7ccccc7c6)nc(-c6ccc7oc8ccccc8c7c6)n5)ccc4c3)ccc2c1 ; c1ccc2cc(-c3nc(-c4ccc5c(c4)sc4ccccc45)nc(-c4cccc5cc(-c6cccc7ccccc67)ccc45)n3)ccc2c1]

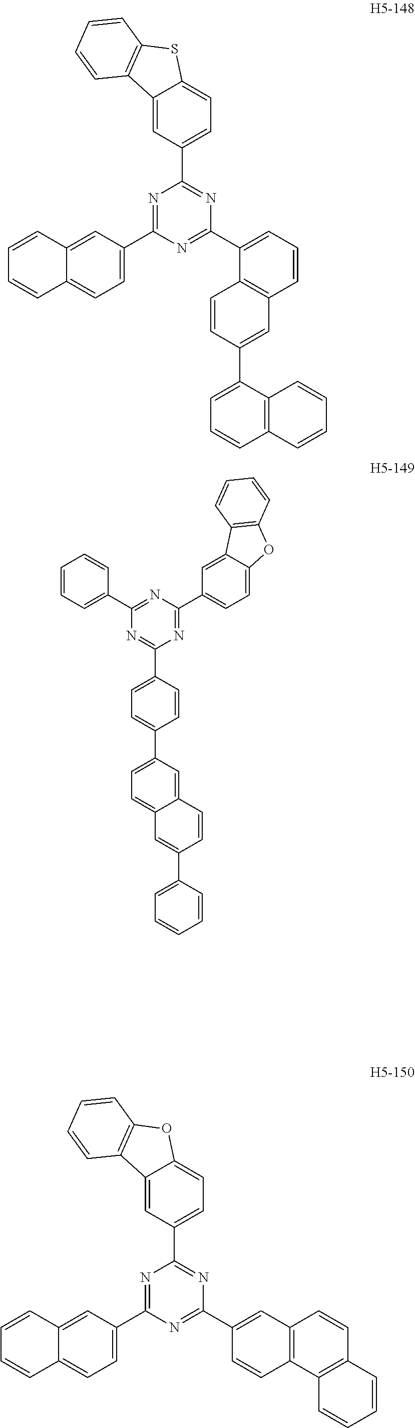

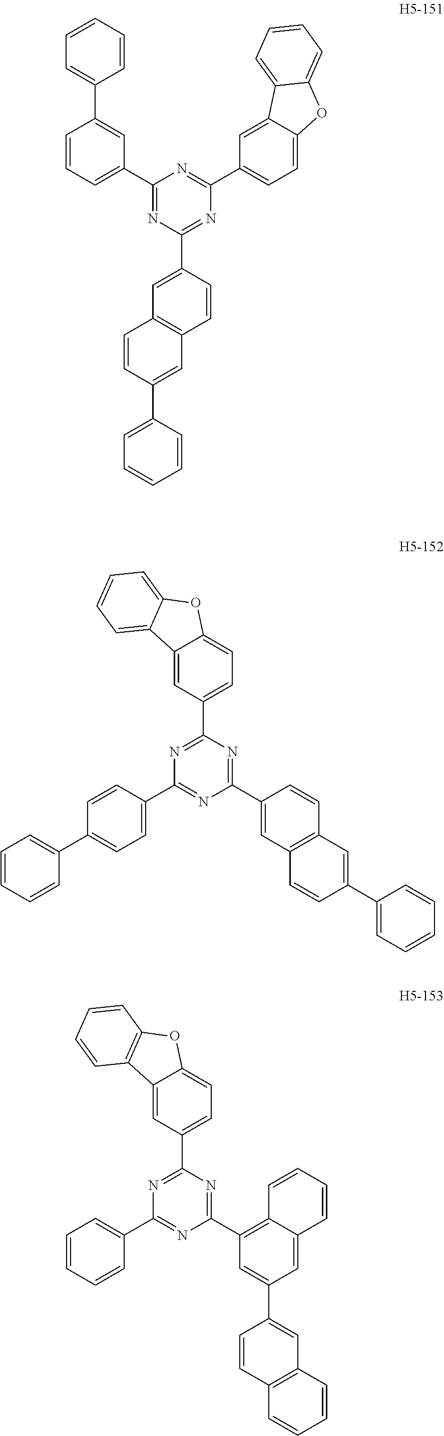 [SMILES: c1ccc(-c2cccc(-c3nc(-c4ccc5cc(-c6ccccc6)ccc5c4)nc(-c4ccc5oc6ccccc6c5c4)n3)c2)cc1 ; c1ccc(-c2ccc(-c3nc(-c4ccc5cc(-c6ccccc6)ccc5c4)nc(-c4ccc5oc6ccccc6c5c4)n3)cc2)cc1 ; c1ccc(-c2nc(-c3ccc4oc5ccccc5c4c3)nc(-c3cc(-c4ccc5ccccc5c4)cc4ccccc34)n2)cc1]

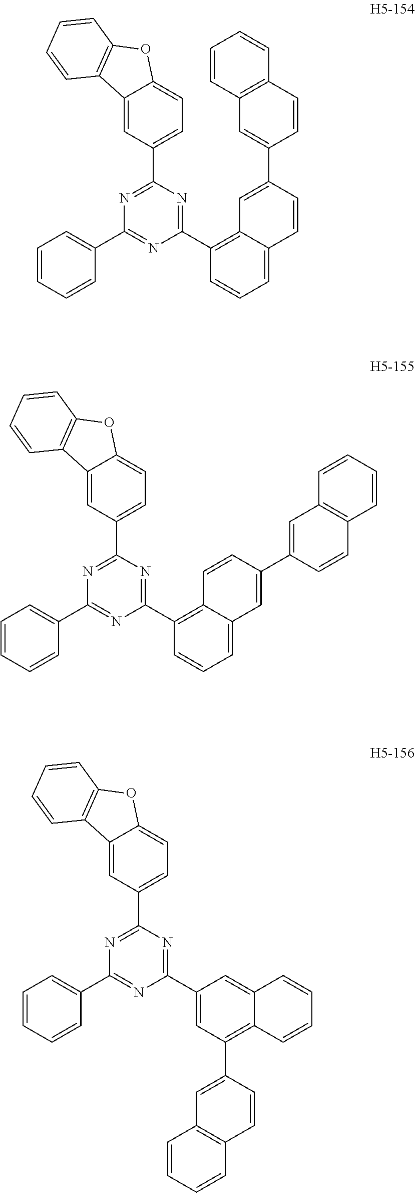 [SMILES: c1ccc(-c2nc(-c3ccc4oc5ccccc5c4c3)nc(-c3cccc4ccc(-c5ccc6ccccc6c5)cc34)n2)cc1 ; c1ccc(-c2nc(-c3ccc4oc5ccccc5c4c3)nc(-c3cccc4cc(-c5ccc6ccccc6c5)ccc34)n2)cc1 ; c1ccc(-c2nc(-c3cc(-c4ccc5ccccc5c4)c4ccccc4c3)nc(-c3ccc4oc5ccccc5c4c3)n2)cc1]

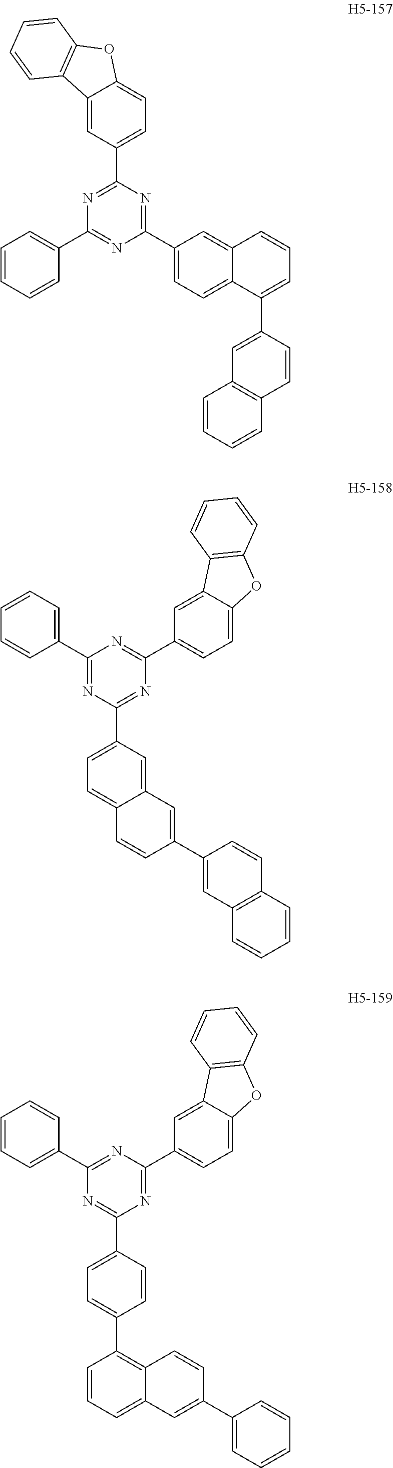 [SMILES: c1ccc(-c2nc(-c3ccc4c(-c5ccc6ccccc6c5)cccc4c3)nc(-c3ccc4oc5ccccc5c4c3)n2)cc1 ; c1ccc(-c2nc(-c3ccc4ccc(-c5ccc6ccccc6c5)cc4c3)nc(-c3ccc4oc5ccccc5c4c3)n2)cc1 ; c1ccc(-c2ccc3c(-c4ccc(-c5nc(-c6ccccc6)nc(-c6ccc7oc8ccccc8c7c6)n5)cc4)cccc3c2)cc1]

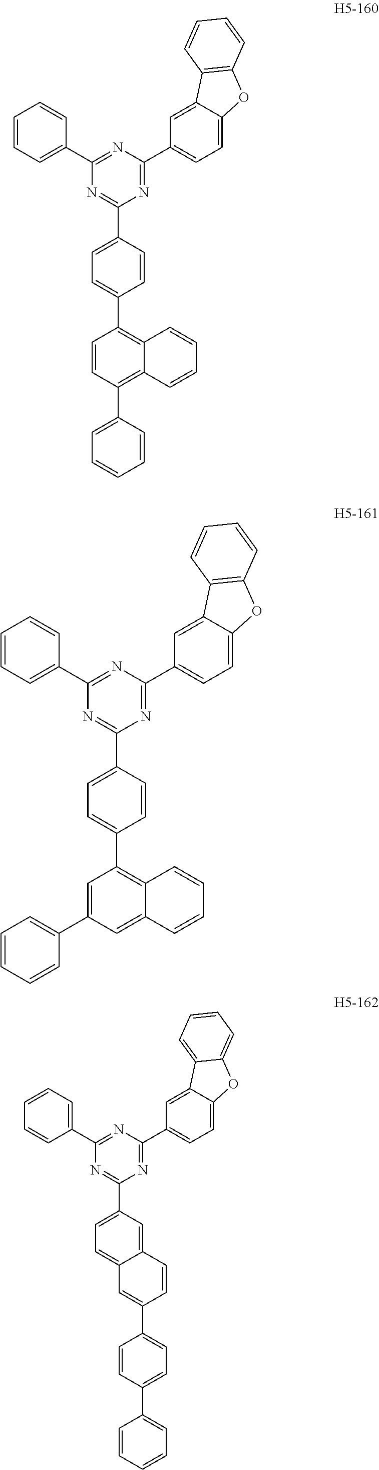 [SMILES: c1ccc(-c2nc(-c3ccc(-c4ccc(-c5ccccc5)c5ccccc45)cc3)nc(-c3ccc4oc5ccccc5c4c3)n2)cc1 ; c1ccc(-c2cc(-c3ccc(-c4nc(-c5ccccc5)nc(-c5ccc6oc7ccccc7c6c5)n4)cc3)c3ccccc3c2)cc1 ; c1ccc(-c2ccc(-c3ccc4cc(-c5nc(-c6ccccc6)nc(-c6ccc7oc8ccccc8c7c6)n5)ccc4c3)cc2)cc1]

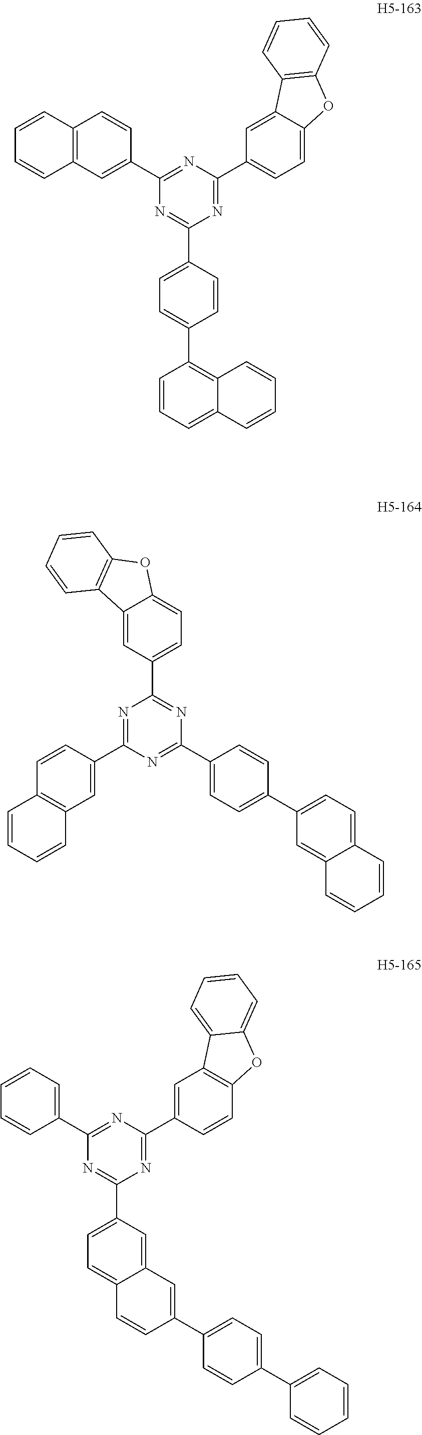 [SMILES: c1ccc2cc(-c3nc(-c4ccc(-c5cccc6ccccc56)cc4)nc(-c4ccc5oc6ccccc6c5c4)n3)ccc2c1 ; c1ccc2cc(-c3ccc(-c4nc(-c5ccc6ccccc6c5)nc(-c5ccc6oc7ccccc7c6c5)n4)cc3)ccc2c1 ; c1ccc(-c2ccc(-c3ccc4ccc(-c5nc(-c6ccccc6)nc(-c6ccc7oc8ccccc8c7c6)n5)cc4c3)cc2)cc1]

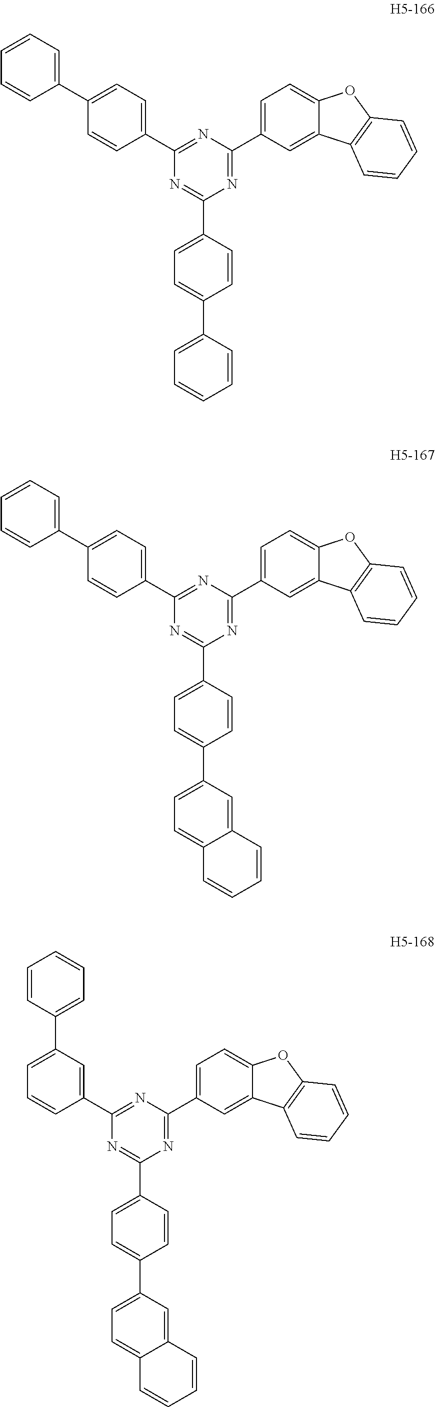 [SMILES: c1ccc(-c2ccc(-c3nc(-c4ccc(-c5ccccc5)cc4)nc(-c4ccc5oc6ccccc6c5c4)n3)cc2)cc1 ; c1ccc(-c2ccc(-c3nc(-c4ccc(-c5ccc6ccccc6c5)cc4)nc(-c4ccc5oc6ccccc6c5c4)n3)cc2)cc1 ; c1ccc(-c2cccc(-c3nc(-c4ccc(-c5ccc6ccccc6c5)cc4)nc(-c4ccc5oc6ccccc6c5c4)n3)c2)cc1]

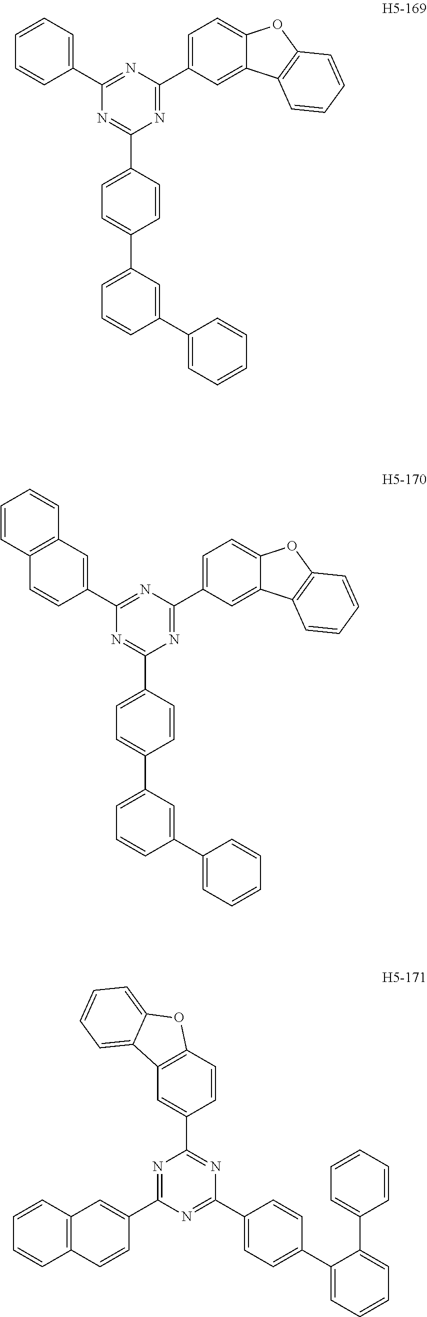 [SMILES: c1ccc(-c2cccc(-c3ccc(-c4nc(-c5ccccc5)nc(-c5ccc6oc7ccccc7c6c5)n4)cc3)c2)cc1 ; c1ccc(-c2cccc(-c3ccc(-c4nc(-c5ccc6ccccc6c5)nc(-c5ccc6oc7ccccc7c6c5)n4)cc3)c2)cc1 ; c1ccc(-c2ccccc2-c2ccc(-c3nc(-c4ccc5ccccc5c4)nc(-c4ccc5oc6ccccc6c5c4)n3)cc2)cc1]

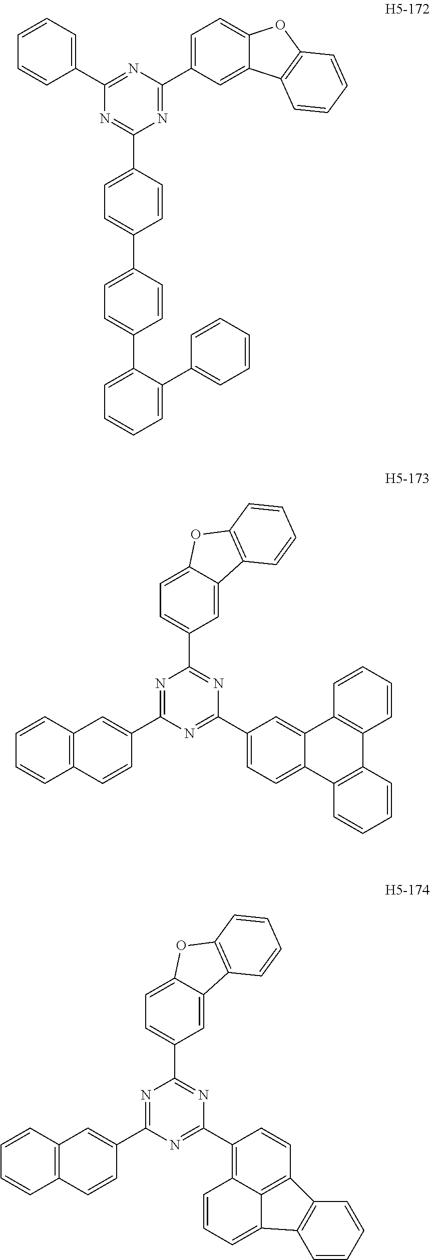 [SMILES: c1ccc(-c2nc(-c3ccc(-c4ccc(-c5ccccc5-c5ccccc5)cc4)cc3)nc(-c3ccc4oc5ccccc5c4c3)n2)cc1 ; c1ccc2cc(-c3nc(-c4ccc5oc6ccccc6c5c4)nc(-c4ccc5c6ccccc6c6ccccc6c5c4)n3)ccc2c1 ; c1ccc2c(c1)-c1cccc3c(-c4nc(-c5ccc6ccccc6c5)nc(-c5ccc6oc7ccccc7c6c5)n4)ccc-2c13]

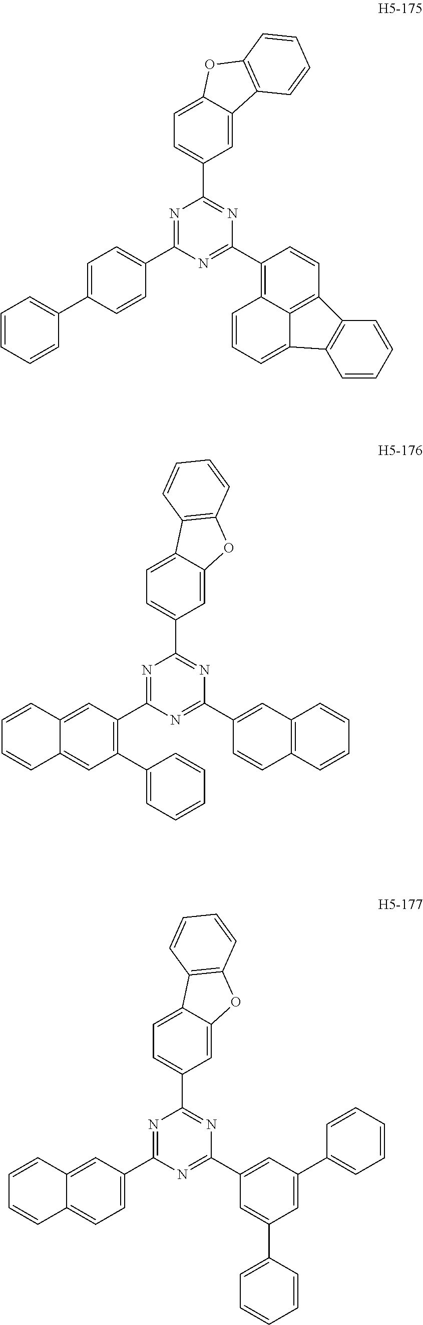 [SMILES: c1ccc(-c2ccc(-c3nc(-c4ccc5oc6ccccc6c5c4)nc(-c4ccc5c6c(cccc46)-c4ccccc4-5)n3)cc2)cc1 ; c1ccc(-c2cc3ccccc3cc2-c2nc(-c3ccc4ccccc4c3)nc(-c3ccc4c(c3)oc3ccccc34)n2)cc1 ; c1ccc(-c2cc(-c3ccccc3)cc(-c3nc(-c4ccc5ccccc5c4)nc(-c4ccc5c(c4)oc4ccccc45)n3)c2)cc1]

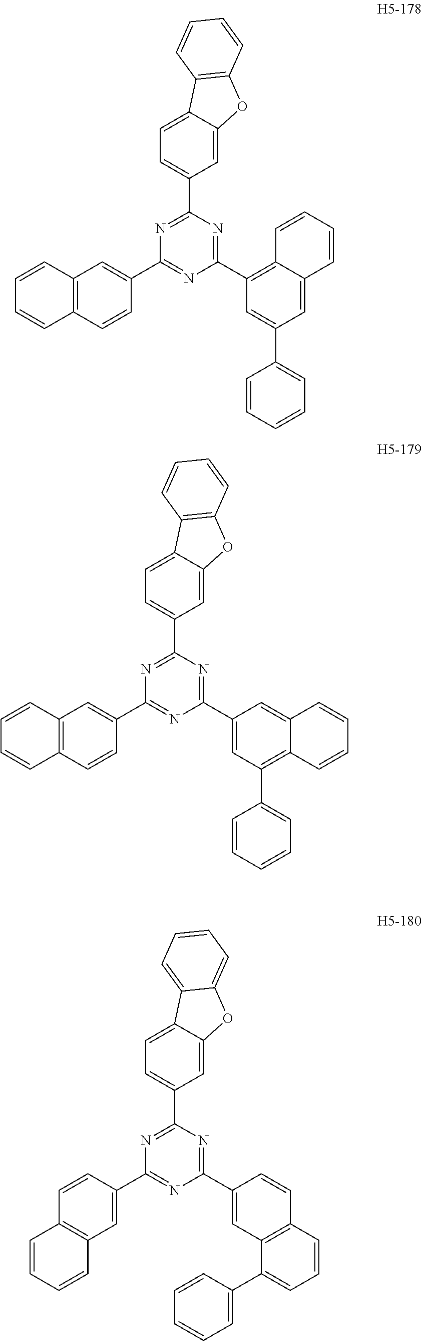 [SMILES: c1ccc(-c2cc(-c3nc(-c4ccc5ccccc5c4)nc(-c4ccc5c(c4)oc4ccccc45)n3)c3ccccc3c2)cc1 ; c1ccc(-c2cc(-c3nc(-c4ccc5ccccc5c4)nc(-c4ccc5c(c4)oc4ccccc45)n3)cc3ccccc23)cc1 ; c1ccc(-c2cccc3ccc(-c4nc(-c5ccc6ccccc6c5)nc(-c5ccc6c(c5)oc5ccccc56)n4)cc23)cc1]

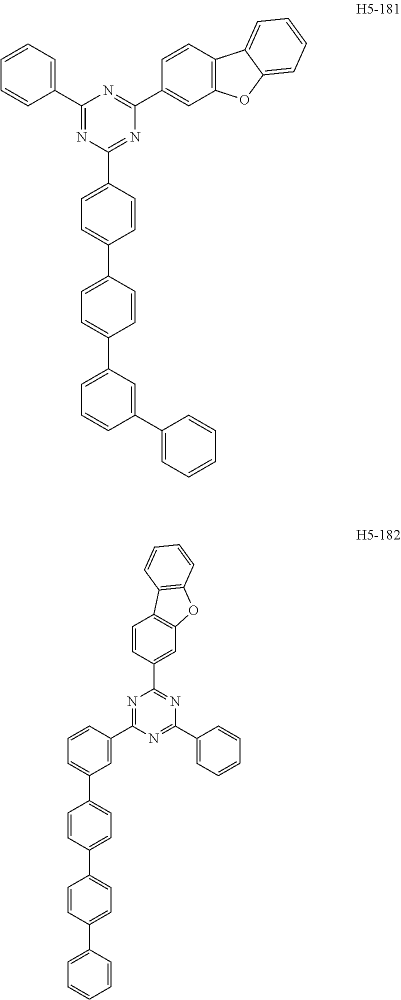 [SMILES: c1ccc(-c2cccc(-c3ccc(-c4ccc(-c5nc(-c6ccccc6)nc(-c6ccc7c(c6)oc6ccccc67)n5)cc4)cc3)c2)cc1 ; c1ccc(-c2ccc(-c3ccc(-c4cccc(-c5nc(-c6ccccc6)nc(-c6ccc7c(c6)oc6ccccc67)n5)c4)cc3)cc2)cc1]

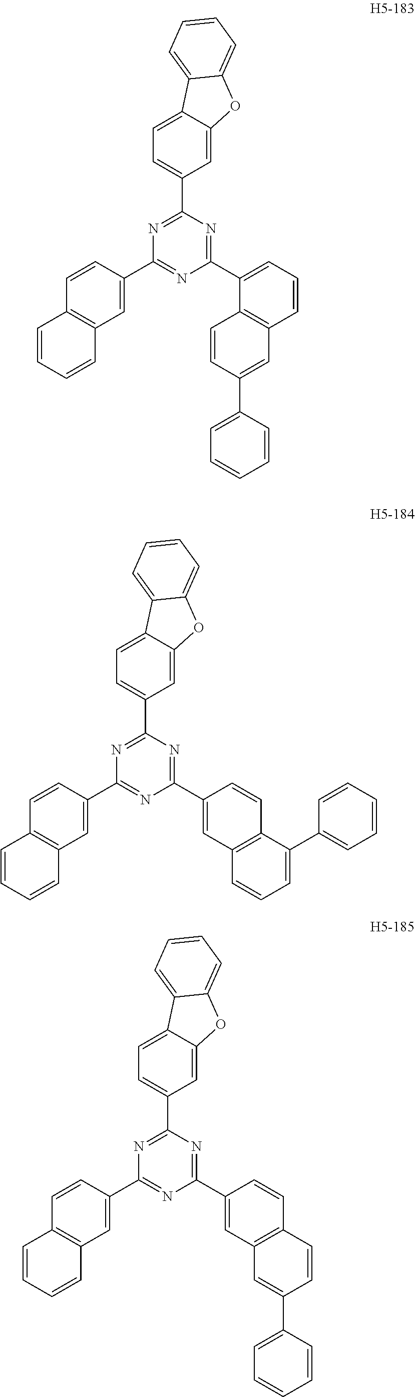 [SMILES: c1ccc(-c2ccc3c(-c4nc(-c5ccc6ccccc6c5)nc(-c5ccc6c(c5)oc5ccccc56)n4)cccc3c2)cc1 ; c1ccc(-c2cccc3cc(-c4nc(-c5ccc6ccccc6c5)nc(-c5ccc6c(c5)oc5ccccc56)n4)ccc23)cc1 ; c1ccc(-c2ccc3ccc(-c4nc(-c5ccc6ccccc6c5)nc(-c5ccc6c(c5)oc5ccccc56)n4)cc3c2)cc1]

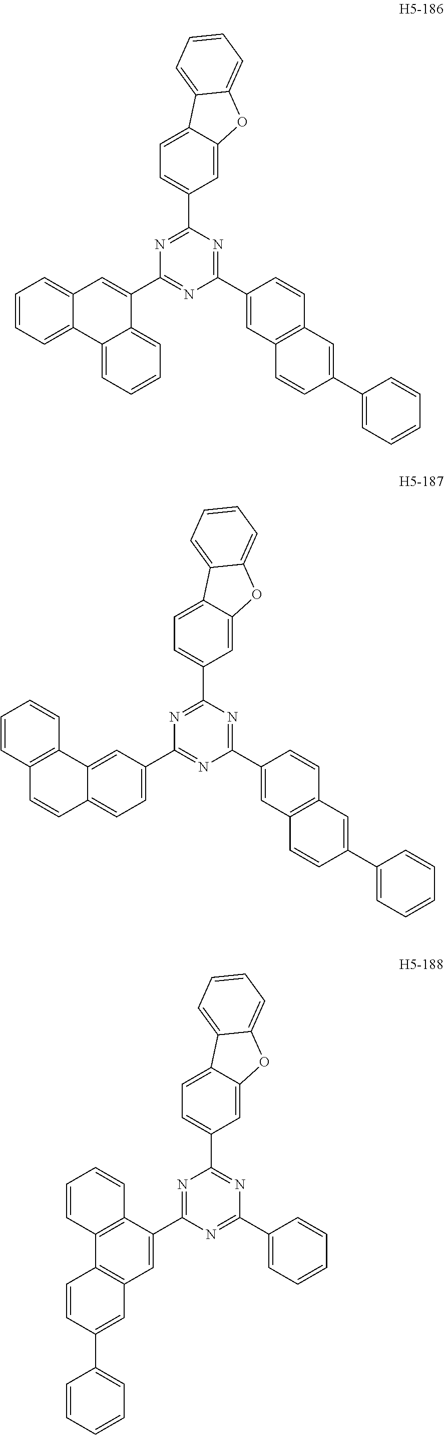 [SMILES: c1ccc(-c2ccc3cc(-c4nc(-c5ccc6c(c5)oc5ccccc56)nc(-c5cc6ccccc6c6ccccc56)n4)ccc3c2)cc1 ; c1ccc(-c2ccc3cc(-c4nc(-c5ccc6c(c5)oc5ccccc56)nc(-c5ccc6ccc7ccccc7c6c5)n4)ccc3c2)cc1 ; c1ccc(-c2ccc3c(c2)cc(-c2nc(-c4ccccc4)nc(-c4ccc5c(c4)oc4ccccc45)n2)c2ccccc23)cc1]

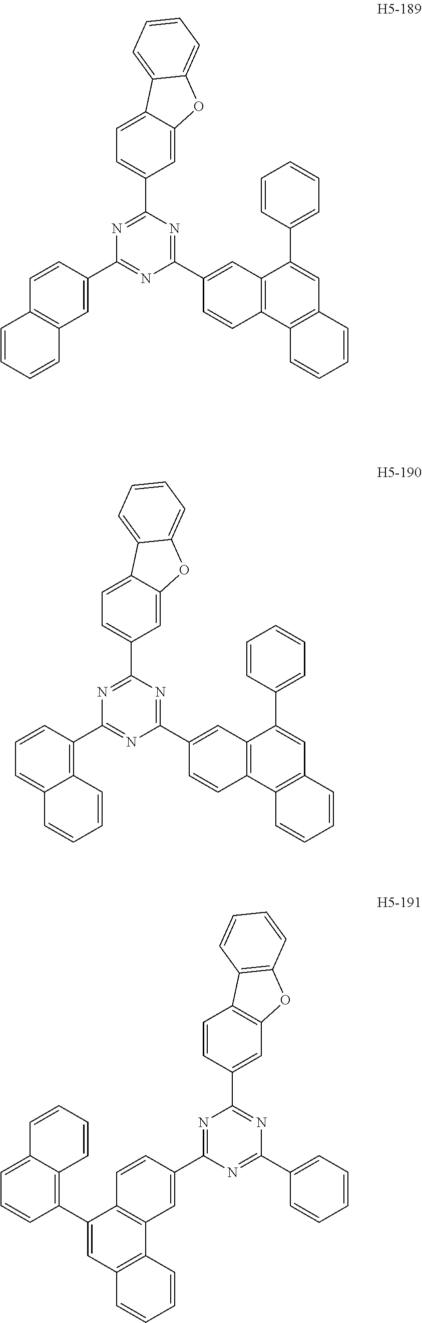 [SMILES: c1ccc(-c2cc3ccccc3c3ccc(-c4nc(-c5ccc6ccccc6c5)nc(-c5ccc6c(c5)oc5ccccc56)n4)cc23)cc1 ; c1ccc(-c2cc3ccccc3c3ccc(-c4nc(-c5ccc6c(c5)oc5ccccc56)nc(-c5cccc6ccccc56)n4)cc23)cc1 ; c1ccc(-c2nc(-c3ccc4c(c3)oc3ccccc34)nc(-c3ccc4c(-c5cccc6ccccc56)cc5ccccc5c4c3)n2)cc1]

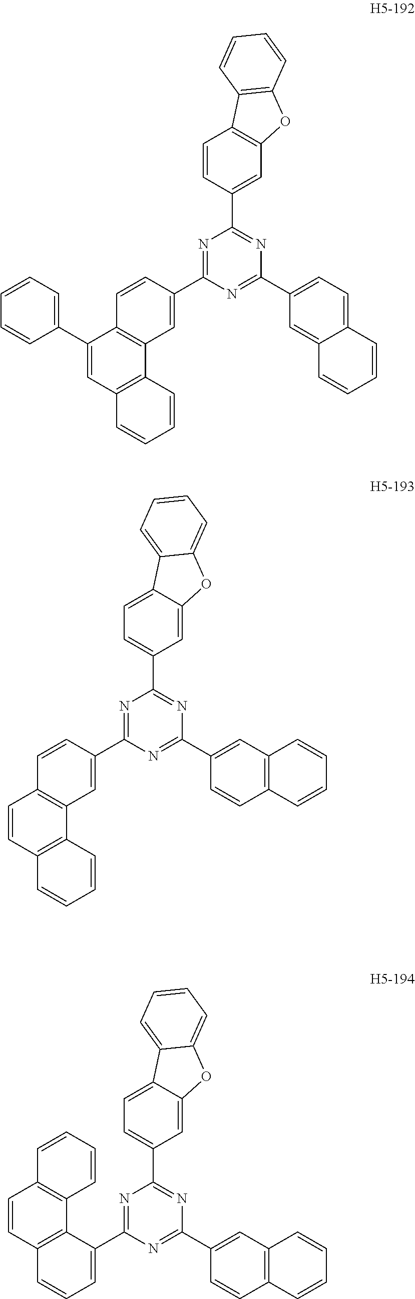 [SMILES: c1ccc(-c2cc3ccccc3c3cc(-c4nc(-c5ccc6ccccc6c5)nc(-c5ccc6c(c5)oc5ccccc56)n4)ccc23)cc1 ; c1ccc2cc(-c3nc(-c4ccc5c(c4)oc4ccccc45)nc(-c4ccc5ccc6ccccc6c5c4)n3)ccc2c1 ; c1ccc2cc(-c3nc(-c4ccc5c(c4)oc4ccccc45)nc(-c4cccc5ccc6ccccc6c45)n3)ccc2c1]

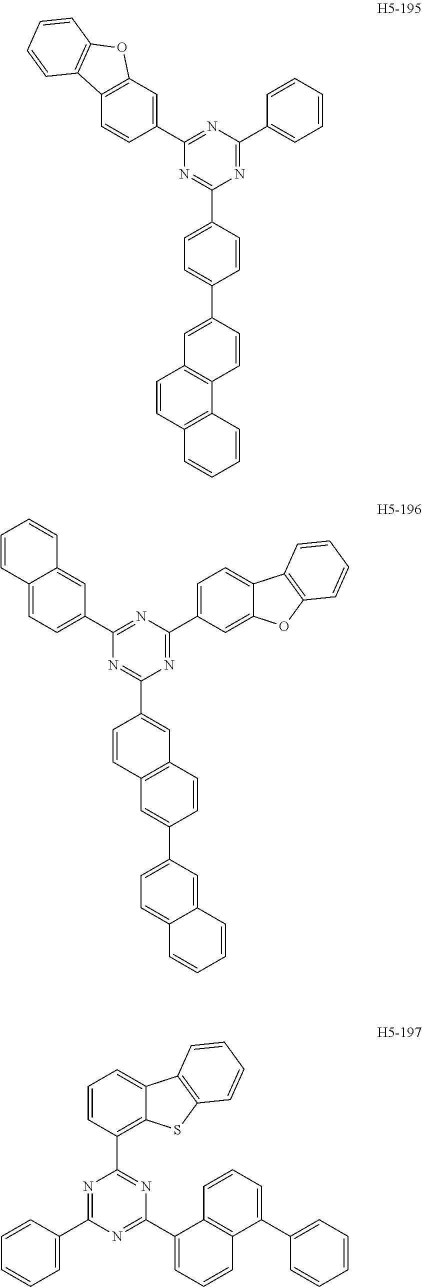 [SMILES: c1ccc(-c2nc(-c3ccc(-c4ccc5c(ccc6ccccc65)c4)cc3)nc(-c3ccc4c(c3)oc3ccccc34)n2)cc1 ; c1ccc2cc(-c3ccc4cc(-c5nc(-c6ccc7ccccc7c6)nc(-c6ccc7c(c6)oc6ccccc67)n5)ccc4c3)ccc2c1 ; c1ccc(-c2nc(-c3cccc4c(-c5ccccc5)cccc34)nc(-c3cccc4c3sc3ccccc34)n2)cc1]

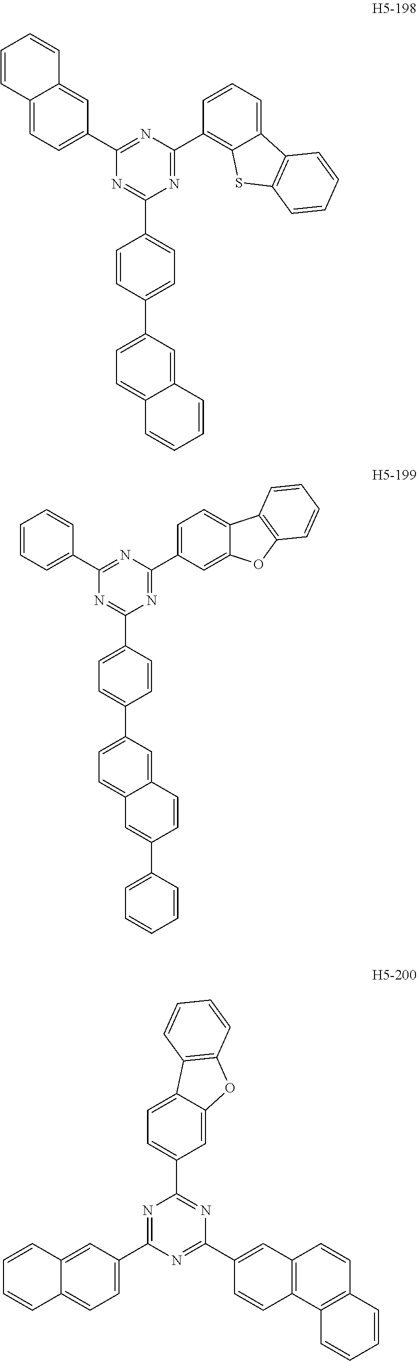 [SMILES: c1ccc2cc(-c3ccc(-c4nc(-c5ccc6ccccc6c5)nc(-c5cccc6c5sc5ccccc56)n4)cc3)ccc2c1 ; c1ccc(-c2ccc3cc(-c4ccc(-c5nc(-c6ccccc6)nc(-c6ccc7c(c6)oc6ccccc67)n5)cc4)ccc3c2)cc1 ; c1ccc2cc(-c3nc(-c4ccc5c(ccc6ccccc65)c4)nc(-c4ccc5c(c4)oc4ccccc45)n3)ccc2c1]

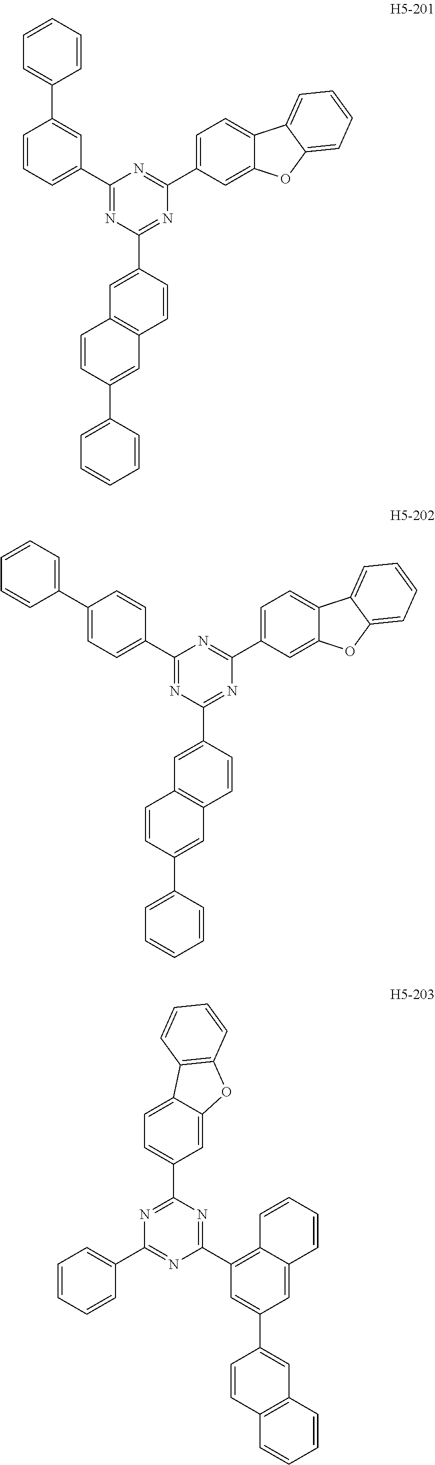 [SMILES: c1ccc(-c2cccc(-c3nc(-c4ccc5cc(-c6ccccc6)ccc5c4)nc(-c4ccc5c(c4)oc4ccccc45)n3)c2)cc1 ; c1ccc(-c2ccc(-c3nc(-c4ccc5cc(-c6ccccc6)ccc5c4)nc(-c4ccc5c(c4)oc4ccccc45)n3)cc2)cc1 ; c1ccc(-c2nc(-c3ccc4c(c3)oc3ccccc34)nc(-c3cc(-c4ccc5ccccc5c4)cc4ccccc34)n2)cc1]

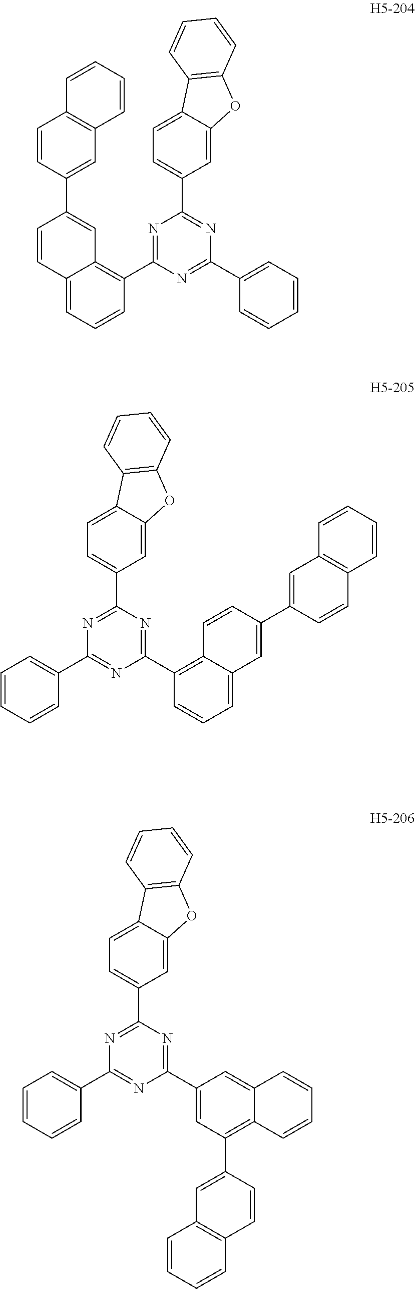 [SMILES: c1ccc(-c2nc(-c3ccc4c(c3)oc3ccccc34)nc(-c3cccc4ccc(-c5ccc6ccccc6c5)cc34)n2)cc1 ; c1ccc(-c2nc(-c3ccc4c(c3)oc3ccccc34)nc(-c3cccc4cc(-c5ccc6ccccc6c5)ccc34)n2)cc1 ; c1ccc(-c2nc(-c3cc(-c4ccc5ccccc5c4)c4ccccc4c3)nc(-c3ccc4c(c3)oc3ccccc34)n2)cc1]

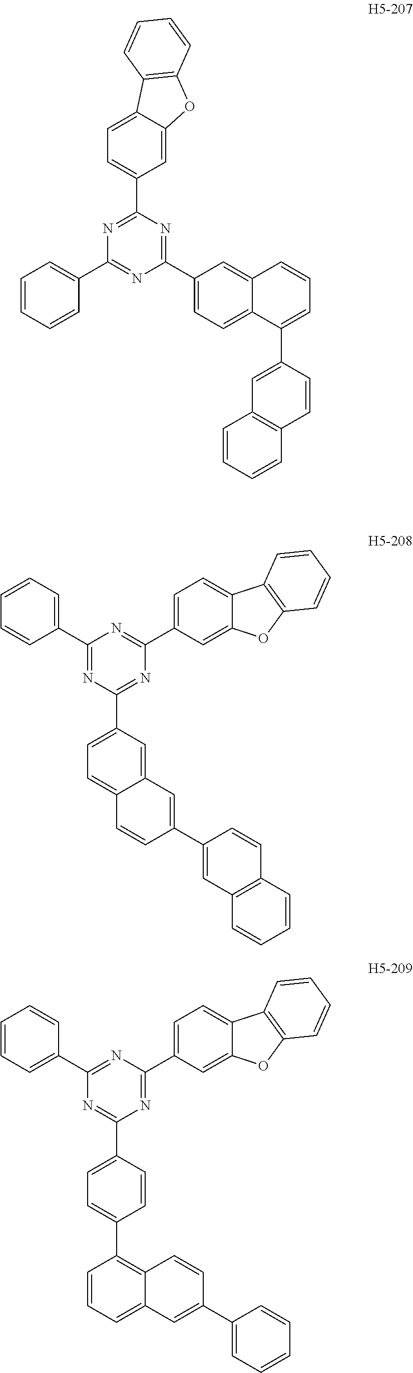

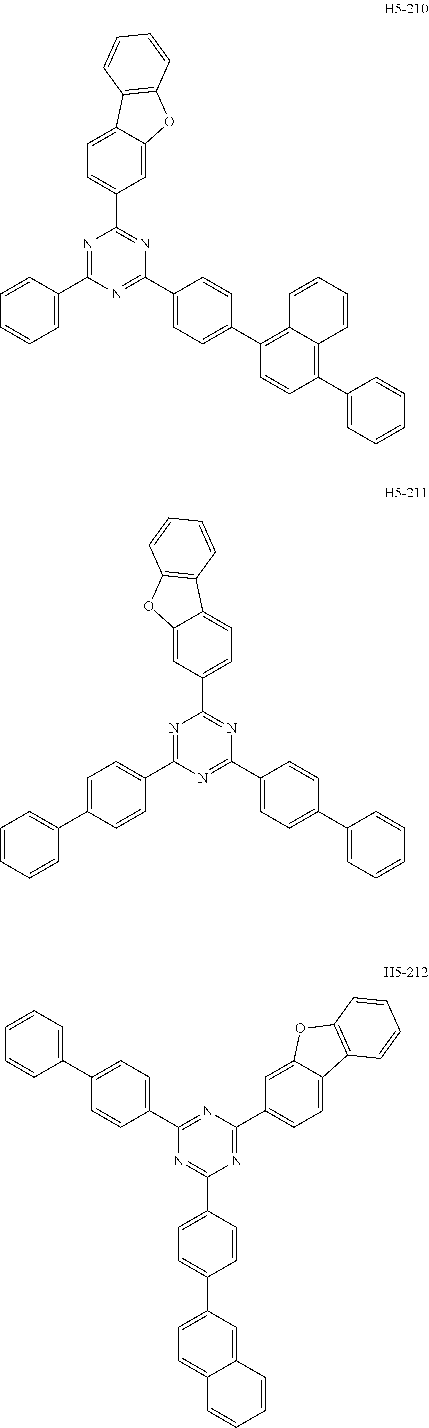 [SMILES: c1ccc(-c2nc(-c3ccc(-c4ccc(-c5ccccc5)c5ccccc45)cc3)nc(-c3ccc4c(c3)oc3ccccc34)n2)cc1 ; c1ccc(-c2ccc(-c3nc(-c4ccc(-c5ccccc5)cc4)nc(-c4ccc5c(c4)oc4ccccc45)n3)cc2)cc1 ; c1ccc(-c2ccc(-c3nc(-c4ccc(-c5ccc6ccccc6c5)cc4)nc(-c4ccc5c(c4)oc4ccccc45)n3)cc2)cc1]

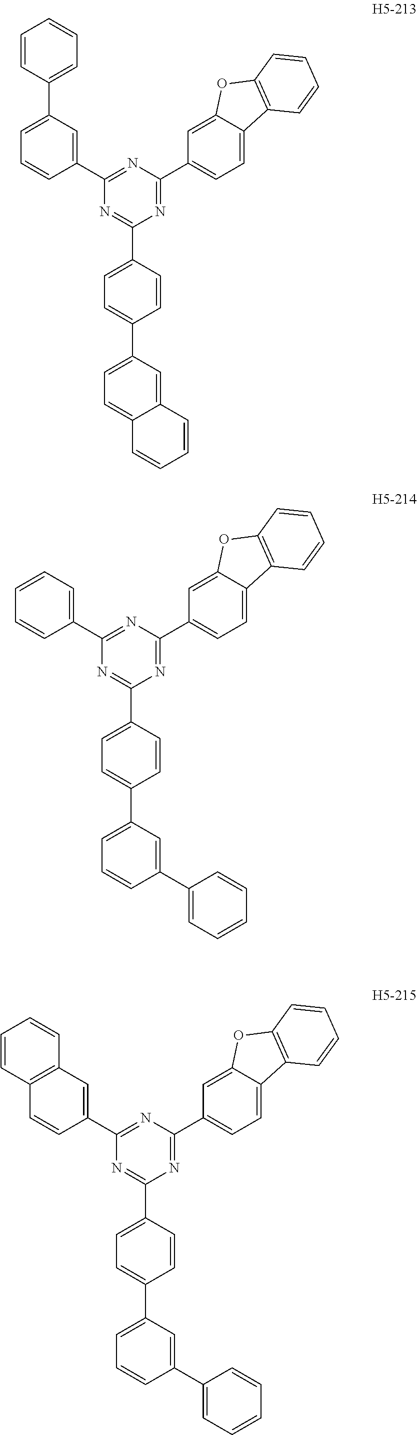 [SMILES: c1ccc(-c2cccc(-c3nc(-c4ccc(-c5ccc6ccccc6c5)cc4)nc(-c4ccc5c(c4)oc4ccccc45)n3)c2)cc1 ; c1ccc(-c2cccc(-c3ccc(-c4nc(-c5ccccc5)nc(-c5ccc6c(c5)oc5ccccc56)n4)cc3)c2)cc1 ; c1ccc(-c2cccc(-c3ccc(-c4nc(-c5ccc6ccccc6c5)nc(-c5ccc6c(c5)oc5ccccc56)n4)cc3)c2)cc1]

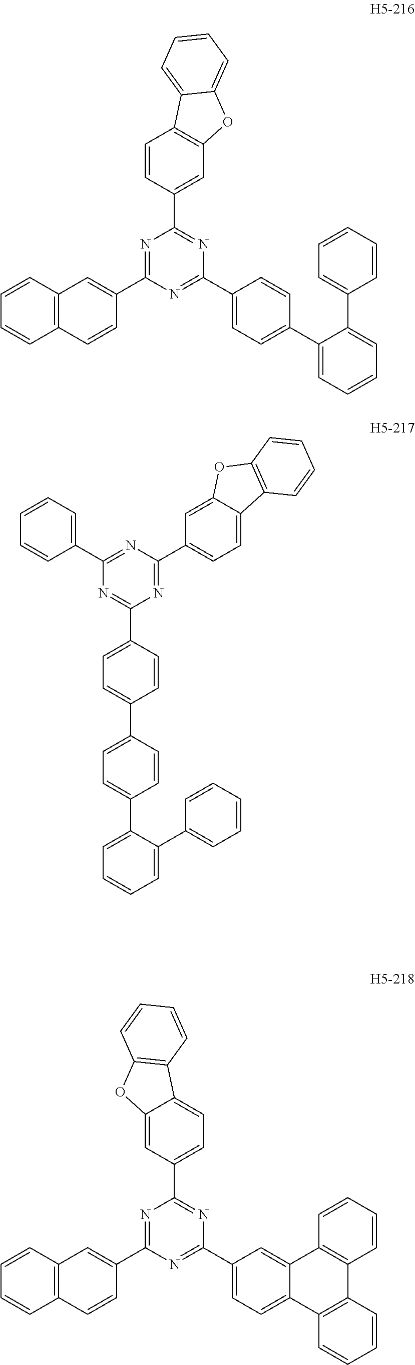 [SMILES: c1ccc(-c2ccccc2-c2ccc(-c3nc(-c4ccc5ccccc5c4)nc(-c4ccc5c(c4)oc4ccccc45)n3)cc2)cc1 ; c1ccc(-c2nc(-c3ccc(-c4ccc(-c5ccccc5-c5ccccc5)cc4)cc3)nc(-c3ccc4c(c3)oc3ccccc34)n2)cc1 ; c1ccc2cc(-c3nc(-c4ccc5c(c4)oc4ccccc45)nc(-c4ccc5c6ccccc6c6ccccc6c5c4)n3)ccc2c1]

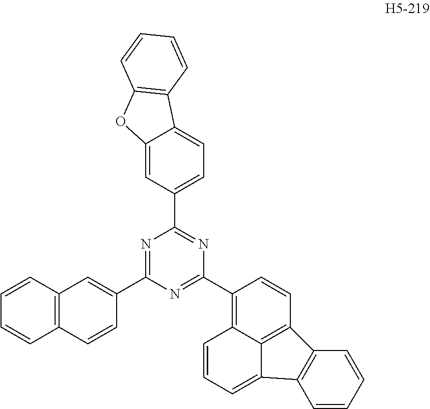 [SMILES: c1ccc2c(c1)-c1cccc3c(-c4nc(-c5ccc6ccccc6c5)nc(-c5ccc6c(c5)oc5ccccc56)n4)ccc-2c13]

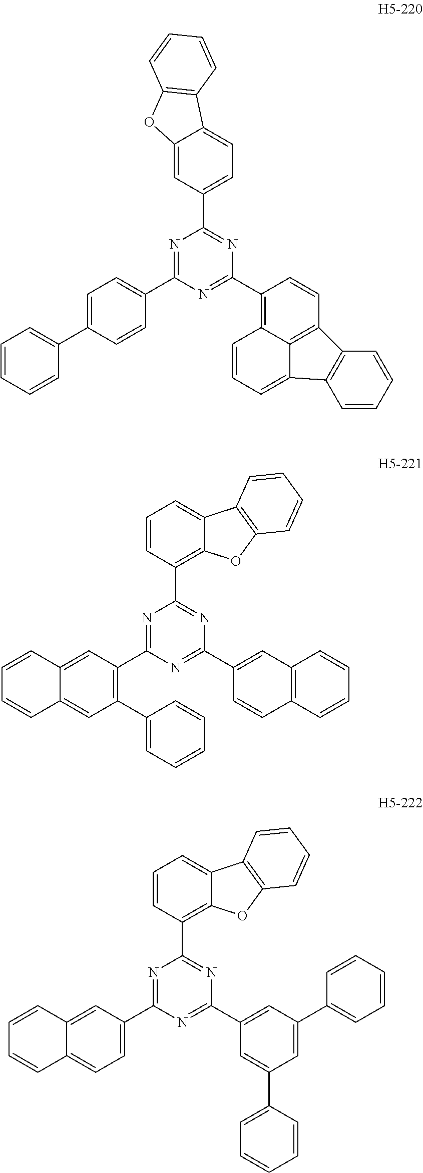 [SMILES: c1ccc(-c2ccc(-c3nc(-c4ccc5c(c4)oc4ccccc45)nc(-c4ccc5c6c(cccc46)-c4ccccc4-5)n3)cc2)cc1 ; c1ccc(-c2cc3ccccc3cc2-c2nc(-c3ccc4ccccc4c3)nc(-c3cccc4c3oc3ccccc34)n2)cc1 ; c1ccc(-c2cc(-c3ccccc3)cc(-c3nc(-c4ccc5ccccc5c4)nc(-c4cccc5c4oc4ccccc45)n3)c2)cc1]

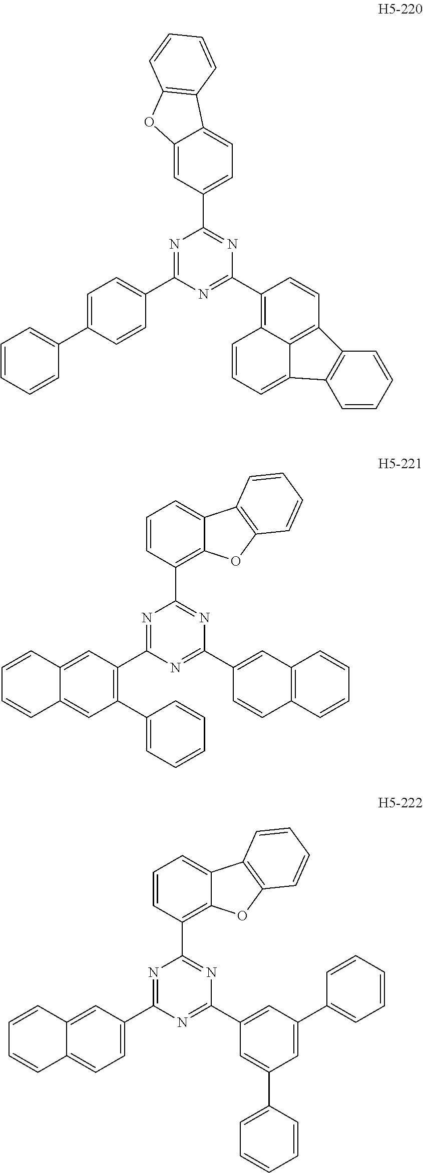 [SMILES: c1ccc(-c2ccc(-c3nc(-c4ccc5c(c4)oc4ccccc45)nc(-c4ccc5c6c(cccc46)-c4ccccc4-5)n3)cc2)cc1 ; c1ccc(-c2cc3ccccc3cc2-c2nc(-c3ccc4ccccc4c3)nc(-c3cccc4c3oc3ccccc34)n2)cc1 ; c1ccc(-c2cc(-c3ccccc3)cc(-c3nc(-c4ccc5ccccc5c4)nc(-c4cccc5c4oc4ccccc45)n3)c2)cc1]

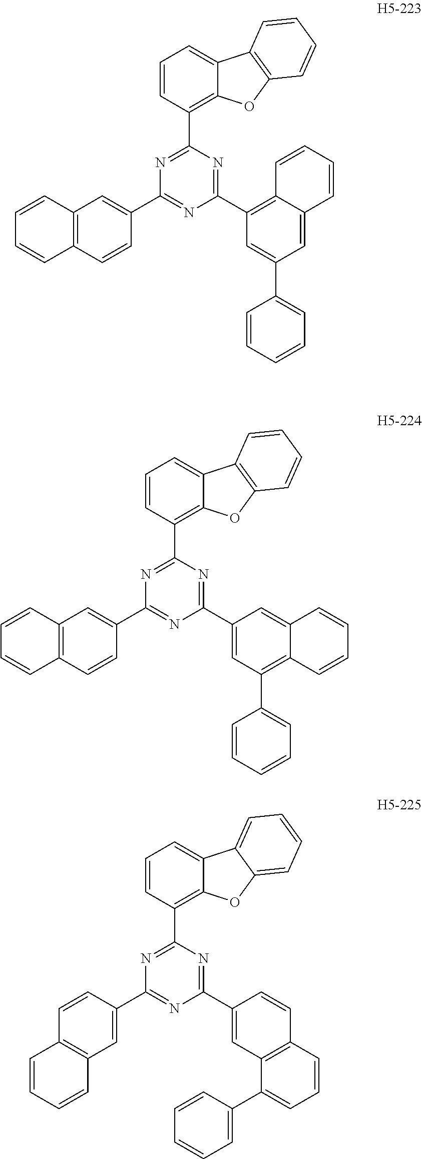

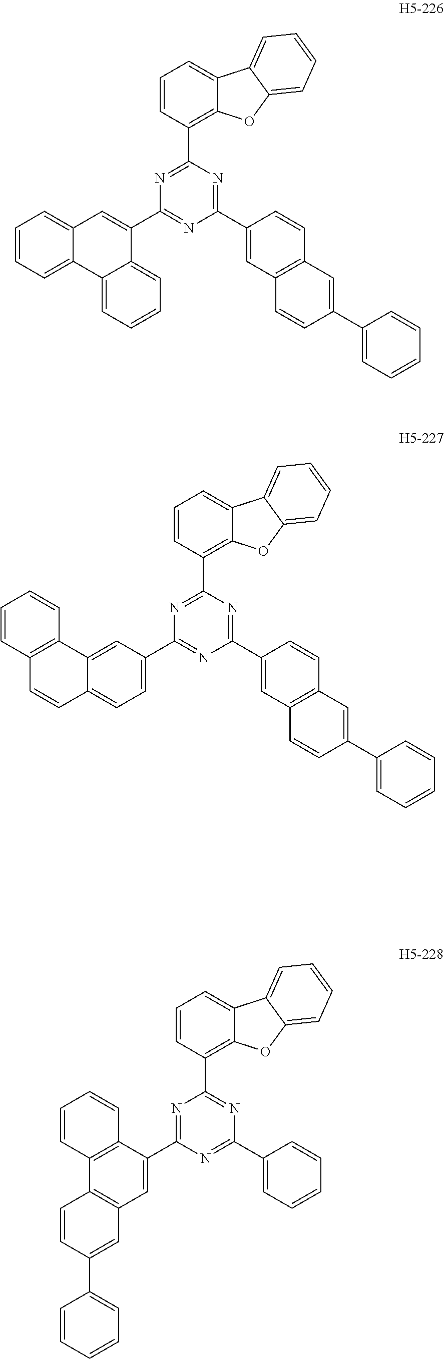 [SMILES: c1ccc(-c2ccc3cc(-c4nc(-c5cc6ccccc6c6ccccc56)nc(-c5cccc6c5oc5ccccc56)n4)ccc3c2)cc1 ; c1ccc(-c2ccc3cc(-c4nc(-c5ccc6ccc7ccccc7c6c5)nc(-c5cccc6c5oc5ccccc56)n4)ccc3c2)cc1 ; c1ccc(-c2ccc3c(c2)cc(-c2nc(-c4ccccc4)nc(-c4cccc5c4oc4ccccc45)n2)c2ccccc23)cc1]

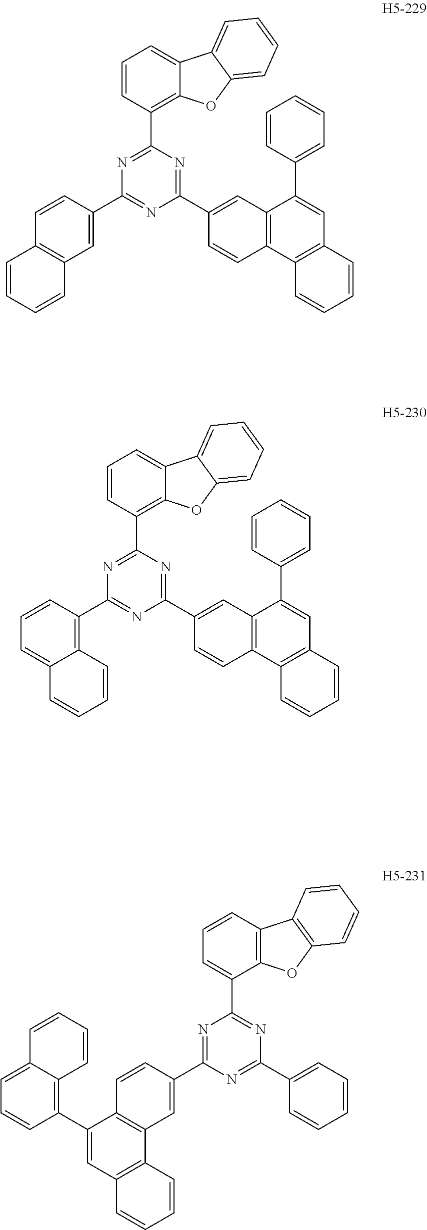 [SMILES: c1ccc(-c2cc3ccccc3c3ccc(-c4nc(-c5ccc6ccccc6c5)nc(-c5cccc6c5oc5ccccc56)n4)cc23)cc1 ; c1ccc(-c2cc3ccccc3c3ccc(-c4nc(-c5cccc6ccccc56)nc(-c5cccc6c5oc5ccccc56)n4)cc23)cc1 ; c1ccc(-c2nc(-c3ccc4c(-c5cccc6ccccc56)cc5ccccc5c4c3)nc(-c3cccc4c3oc3ccccc34)n2)cc1]

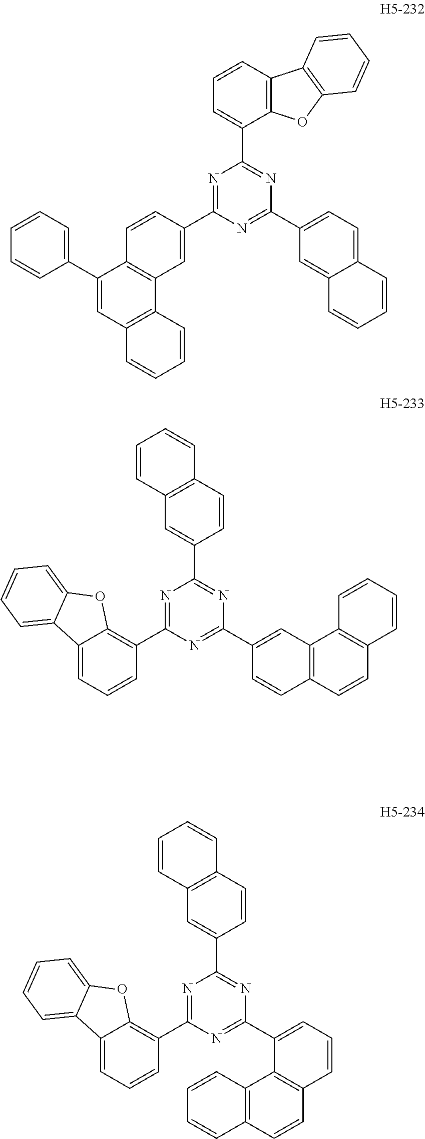 [SMILES: c1ccc(-c2cc3ccccc3c3cc(-c4nc(-c5ccc6ccccc6c5)nc(-c5cccc6c5oc5ccccc56)n4)ccc23)cc1 ; c1ccc2cc(-c3nc(-c4ccc5ccc6ccccc6c5c4)nc(-c4cccc5c4oc4ccccc45)n3)ccc2c1 ; c1ccc2cc(-c3nc(-c4cccc5c4oc4ccccc45)nc(-c4cccc5ccc6ccccc6c45)n3)ccc2c1]

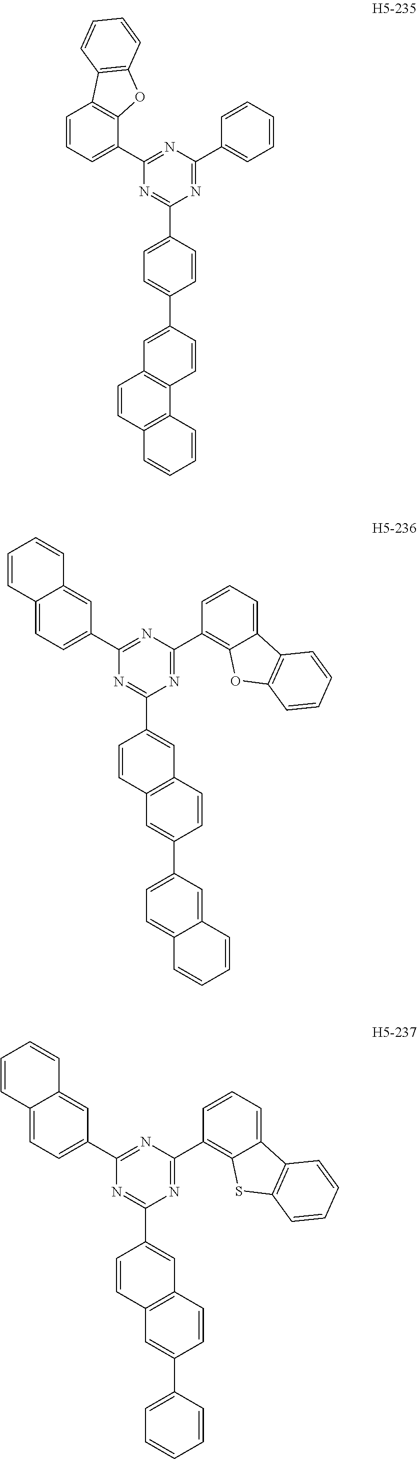 [SMILES: c1ccc(-c2nc(-c3ccc(-c4ccc5c(ccc6ccccc65)c4)cc3)nc(-c3cccc4c3oc3ccccc34)n2)cc1 ; c1ccc2cc(-c3ccc4cc(-c5nc(-c6ccc7ccccc7c6)nc(-c6cccc7c6oc6ccccc67)n5)ccc4c3)ccc2c1 ; c1ccc(-c2ccc3cc(-c4nc(-c5ccc6ccccc6c5)nc(-c5cccc6c5sc5ccccc56)n4)ccc3c2)cc1]

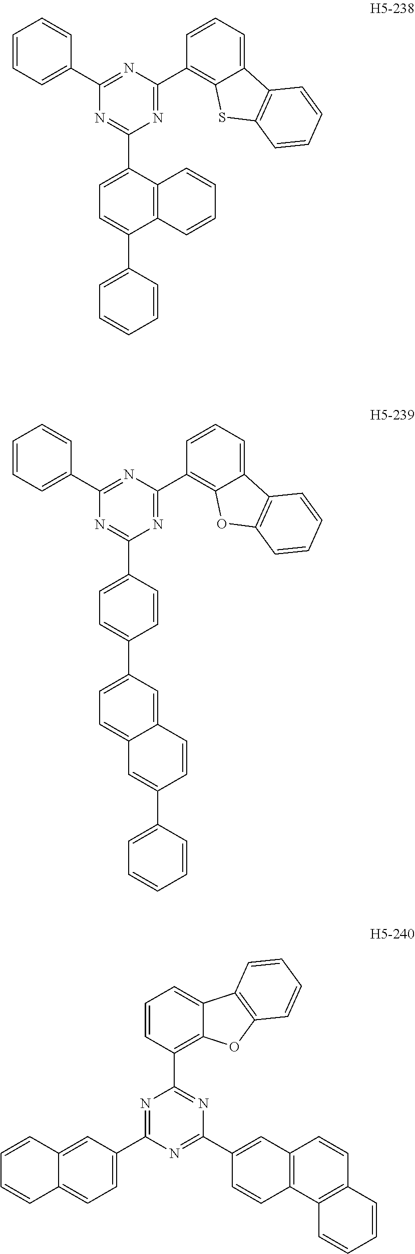 [SMILES: c1ccc(-c2nc(-c3ccc(-c4ccccc4)c4ccccc34)nc(-c3cccc4c3sc3ccccc34)n2)cc1 ; c1ccc(-c2ccc3cc(-c4ccc(-c5nc(-c6ccccc6)nc(-c6cccc7c6oc6ccccc67)n5)cc4)ccc3c2)cc1 ; c1ccc2cc(-c3nc(-c4ccc5c(ccc6ccccc65)c4)nc(-c4cccc5c4oc4ccccc45)n3)ccc2c1]

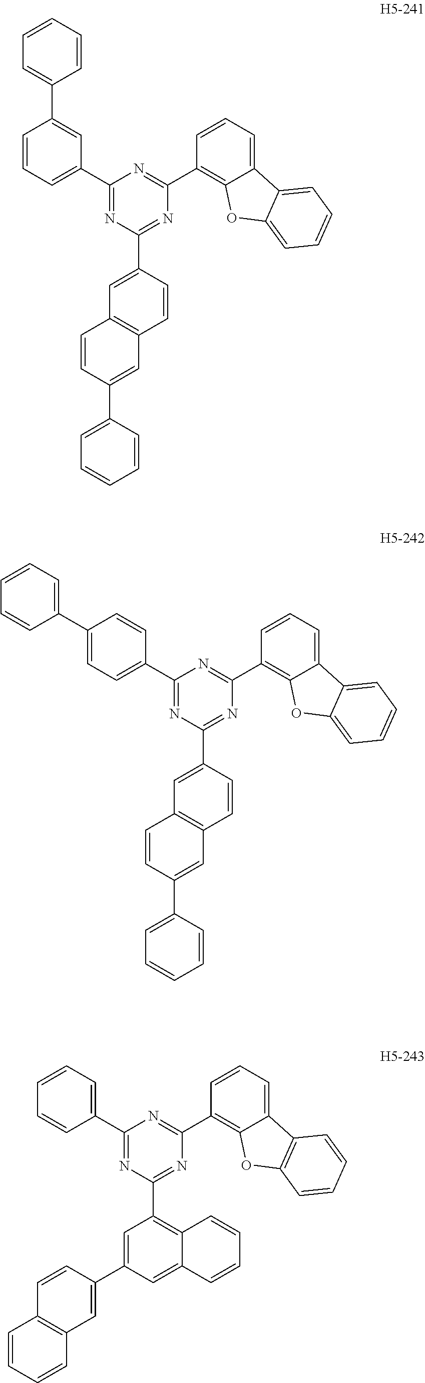 [SMILES: c1ccc(-c2cccc(-c3nc(-c4ccc5cc(-c6ccccc6)ccc5c4)nc(-c4cccc5c4oc4ccccc45)n3)c2)cc1 ; c1ccc(-c2ccc(-c3nc(-c4ccc5cc(-c6ccccc6)ccc5c4)nc(-c4cccc5c4oc4ccccc45)n3)cc2)cc1 ; c1ccc(-c2nc(-c3cc(-c4ccc5ccccc5c4)cc4ccccc34)nc(-c3cccc4c3oc3ccccc34)n2)cc1]

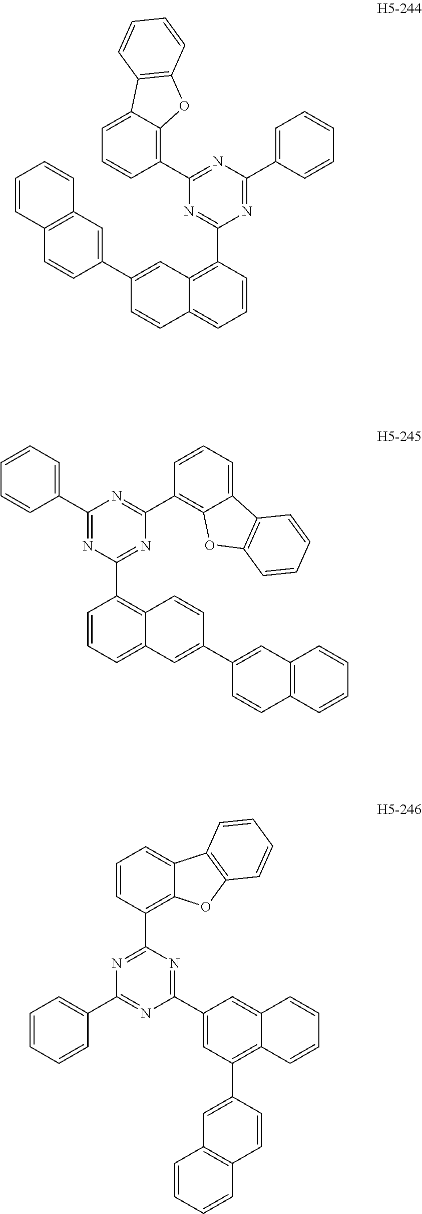 [SMILES: c1ccc(-c2nc(-c3cccc4ccc(-c5ccc6ccccc6c5)cc34)nc(-c3cccc4c3oc3ccccc34)n2)cc1 ; c1ccc(-c2nc(-c3cccc4cc(-c5ccc6ccccc6c5)ccc34)nc(-c3cccc4c3oc3ccccc34)n2)cc1 ; c1ccc(-c2nc(-c3cc(-c4ccc5ccccc5c4)c4ccccc4c3)nc(-c3cccc4c3oc3ccccc34)n2)cc1]

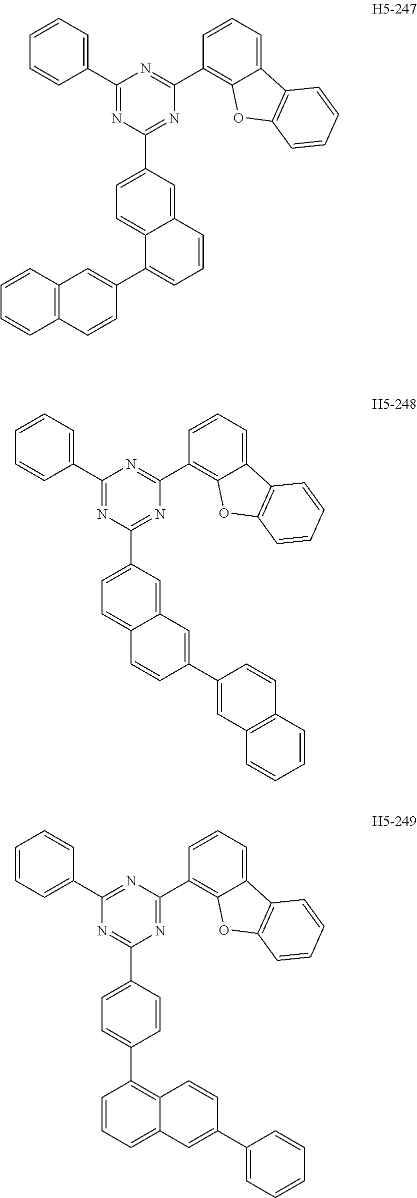 [SMILES: c1ccc(-c2nc(-c3ccc4c(-c5ccc6ccccc6c5)cccc4c3)nc(-c3cccc4c3oc3ccccc34)n2)cc1 ; c1ccc(-c2nc(-c3ccc4ccc(-c5ccc6ccccc6c5)cc4c3)nc(-c3cccc4c3oc3ccccc34)n2)cc1 ; c1ccc(-c2ccc3c(-c4ccc(-c5nc(-c6ccccc6)nc(-c6cccc7c6oc6ccccc67)n5)cc4)cccc3c2)cc1]

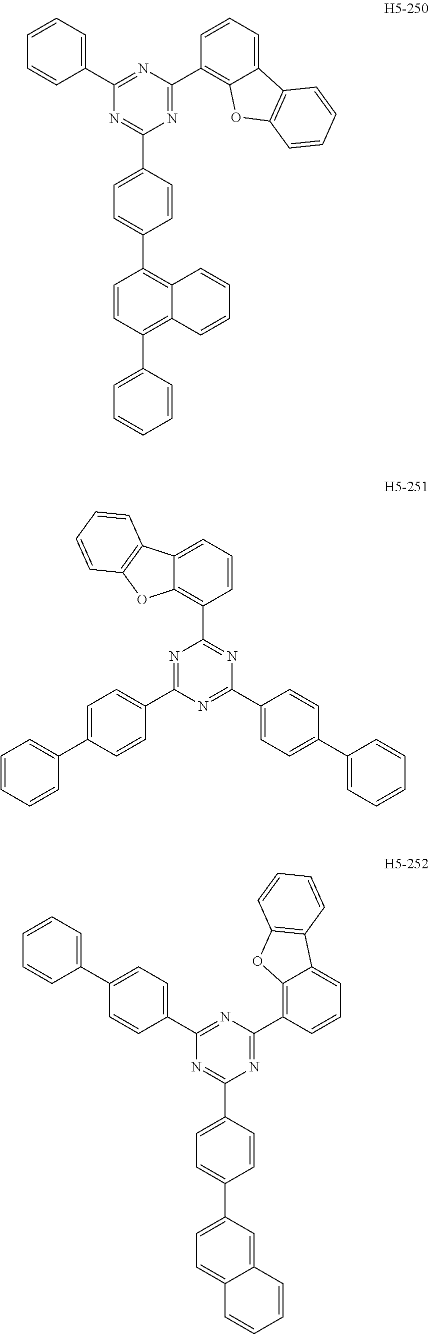 [SMILES: c1ccc(-c2nc(-c3ccc(-c4ccc(-c5ccccc5)c5ccccc45)cc3)nc(-c3cccc4c3oc3ccccc34)n2)cc1 ; c1ccc(-c2ccc(-c3nc(-c4ccc(-c5ccccc5)cc4)nc(-c4cccc5c4oc4ccccc45)n3)cc2)cc1 ; c1ccc(-c2ccc(-c3nc(-c4ccc(-c5ccc6ccccc6c5)cc4)nc(-c4cccc5c4oc4ccccc45)n3)cc2)cc1]

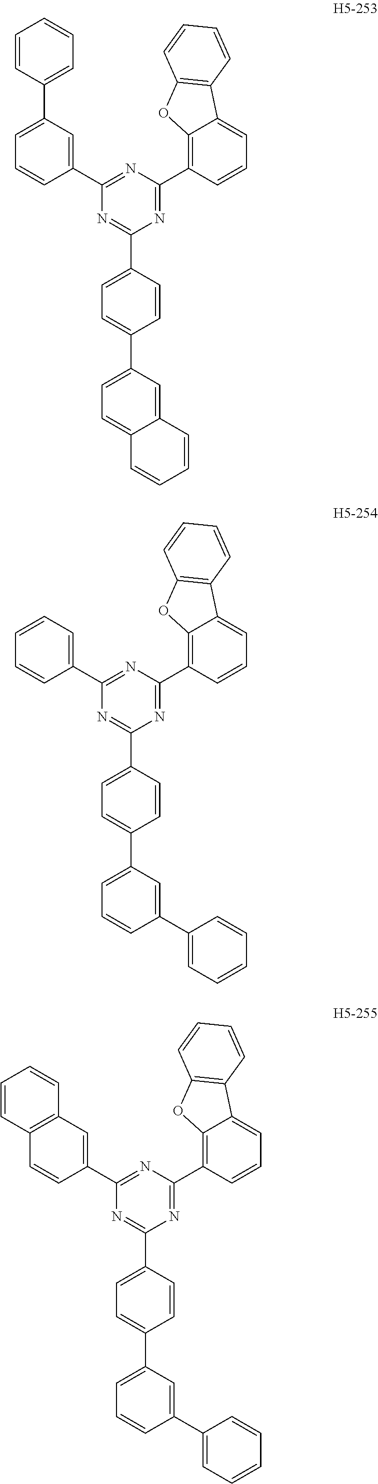

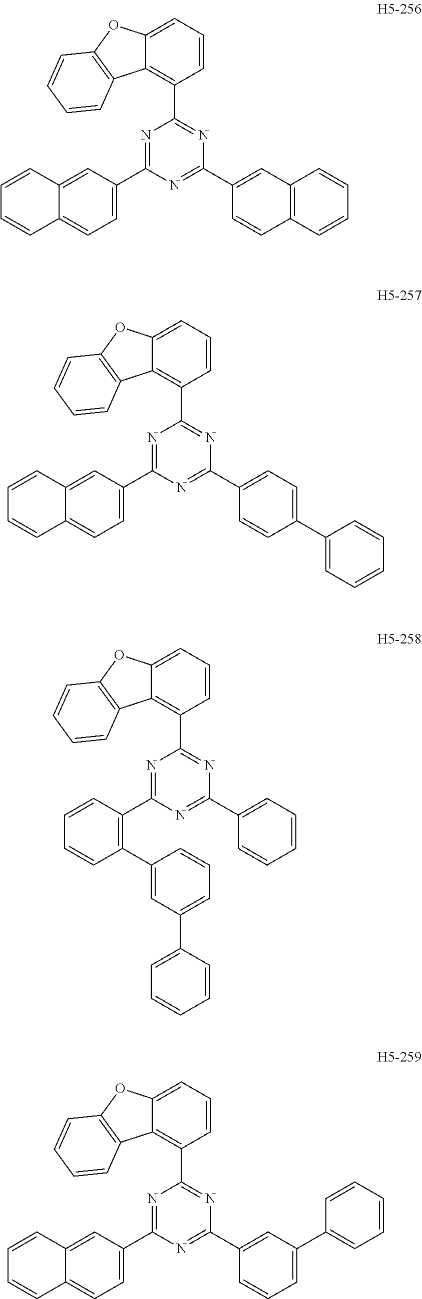 [SMILES: c1ccc2cc(-c3nc(-c4ccc5ccccc5c4)nc(-c4cccc5oc6ccccc6c45)n3)ccc2c1 ; c1ccc(-c2ccc(-c3nc(-c4ccc5ccccc5c4)nc(-c4cccc5oc6ccccc6c45)n3)cc2)cc1 ; c1ccc(-c2cccc(-c3ccccc3-c3nc(-c4ccccc4)nc(-c4cccc5oc6ccccc6c45)n3)c2)cc1 ; c1ccc(-c2cccc(-c3nc(-c4ccc5ccccc5c4)nc(-c4cccc5oc6ccccc6c45)n3)c2)cc1]

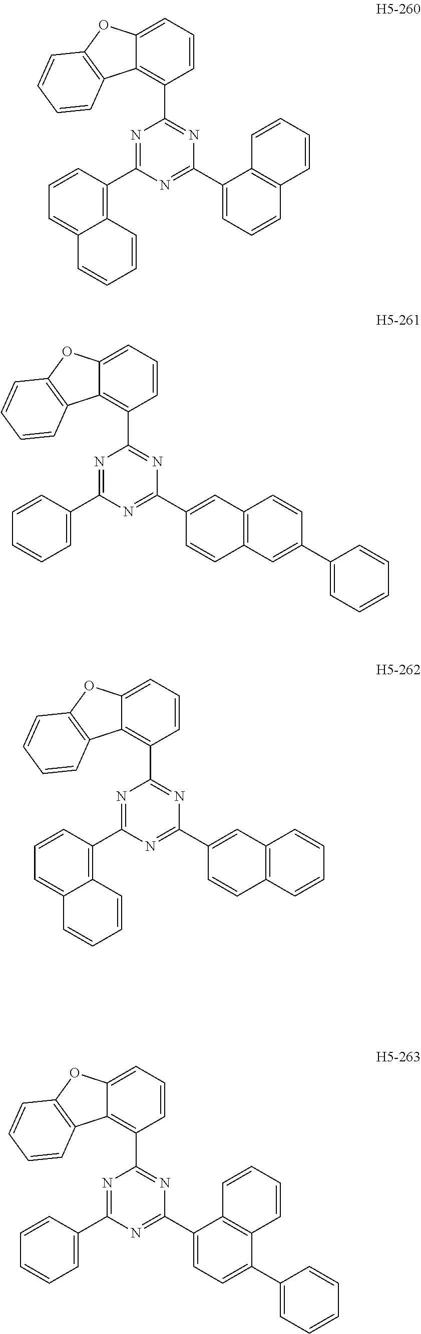 [SMILES: c1ccc2c(-c3nc(-c4cccc5ccccc45)nc(-c4cccc5oc6ccccc6c45)n3)cccc2c1 ; c1ccc(-c2ccc3cc(-c4nc(-c5ccccc5)nc(-c5cccc6oc7ccccc7c56)n4)ccc3c2)cc1 ; c1ccc2cc(-c3nc(-c4cccc5ccccc45)nc(-c4cccc5oc6ccccc6c45)n3)ccc2c1 ; c1ccc(-c2nc(-c3ccc(-c4ccccc4)c4ccccc34)nc(-c3cccc4oc5ccccc5c34)n2)cc1]

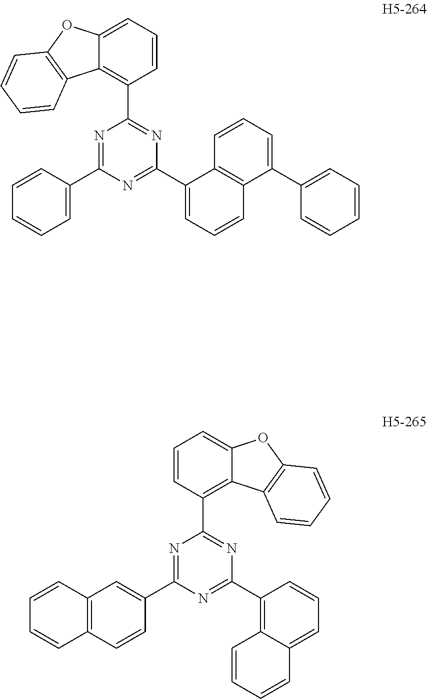 [SMILES: c1ccc(-c2nc(-c3cccc4c(-c5ccccc5)cccc34)nc(-c3cccc4oc5ccccc5c34)n2)cc1 ; c1ccc2cc(-c3nc(-c4cccc5ccccc45)nc(-c4cccc5oc6ccccc6c45)n3)ccc2c1]

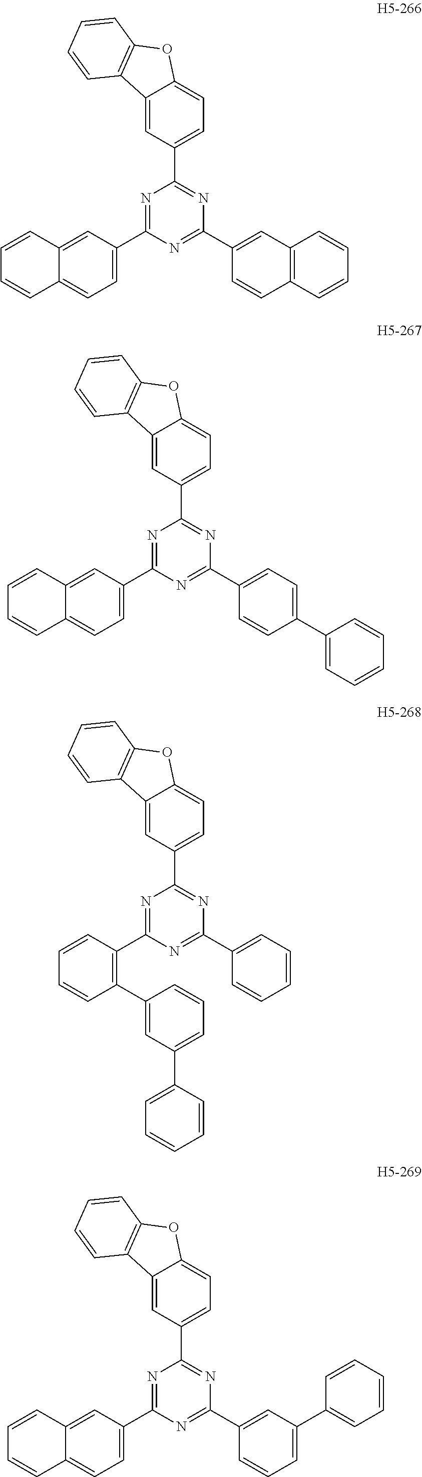 [SMILES: c1ccc2cc(-c3nc(-c4ccc5ccccc5c4)nc(-c4ccc5oc6ccccc6c5c4)n3)ccc2c1 ; c1ccc(-c2ccc(-c3nc(-c4ccc5ccccc5c4)nc(-c4ccc5oc6ccccc6c5c4)n3)cc2)cc1 ; c1ccc(-c2cccc(-c3ccccc3-c3nc(-c4ccccc4)nc(-c4ccc5oc6ccccc6c5c4)n3)c2)cc1 ; c1ccc(-c2cccc(-c3nc(-c4ccc5ccccc5c4)nc(-c4ccc5oc6ccccc6c5c4)n3)c2)cc1]

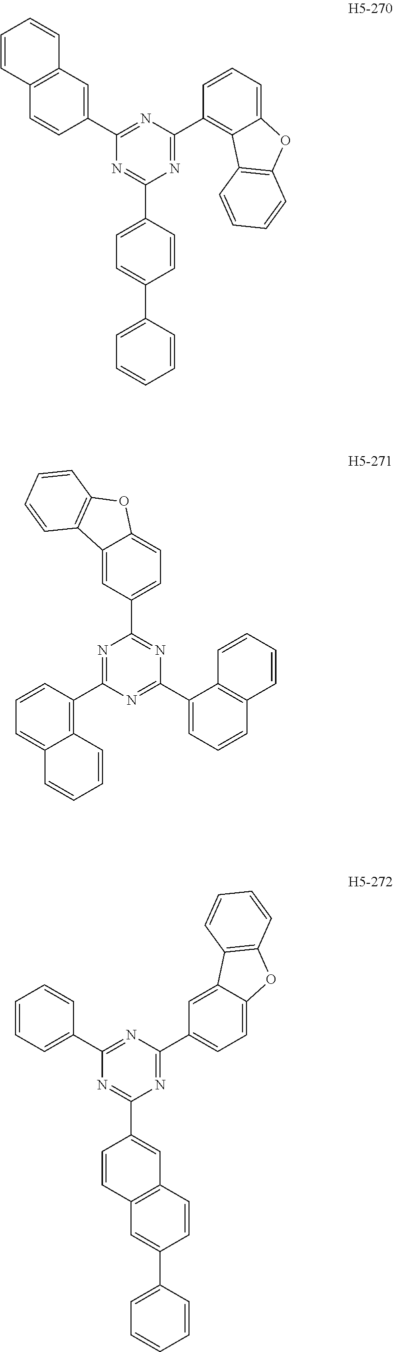 [SMILES: c1ccc(-c2ccc(-c3nc(-c4ccc5ccccc5c4)nc(-c4cccc5oc6ccccc6c45)n3)cc2)cc1 ; c1ccc2c(-c3nc(-c4ccc5oc6ccccc6c5c4)nc(-c4cccc5ccccc45)n3)cccc2c1 ; c1ccc(-c2ccc3cc(-c4nc(-c5ccccc5)nc(-c5ccc6oc7ccccc7c6c5)n4)ccc3c2)cc1]

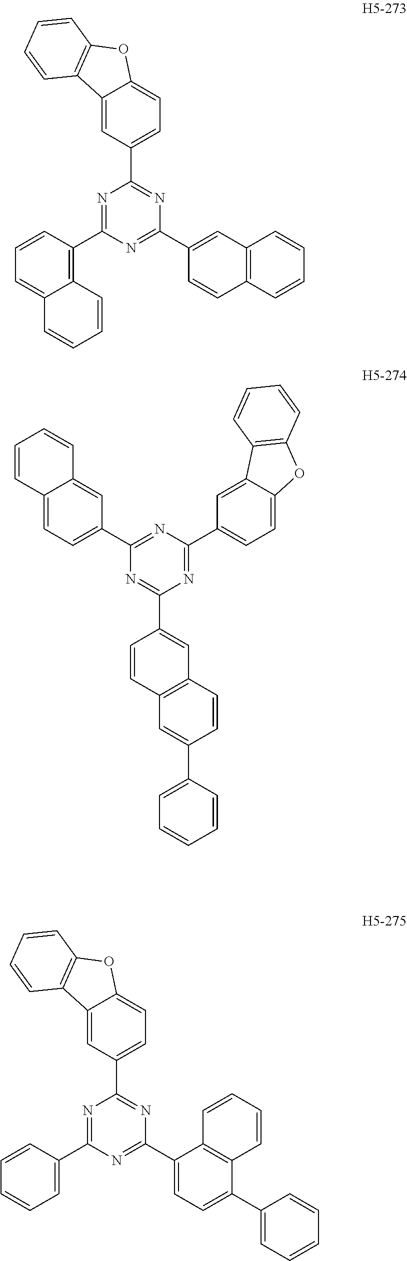 [SMILES: c1ccc2cc(-c3nc(-c4ccc5oc6ccccc6c5c4)nc(-c4cccc5ccccc45)n3)ccc2c1 ; c1ccc(-c2ccc3cc(-c4nc(-c5ccc6ccccc6c5)nc(-c5ccc6oc7ccccc7c6c5)n4)ccc3c2)cc1 ; c1ccc(-c2nc(-c3ccc4oc5ccccc5c4c3)nc(-c3ccc(-c4ccccc4)c4ccccc34)n2)cc1]

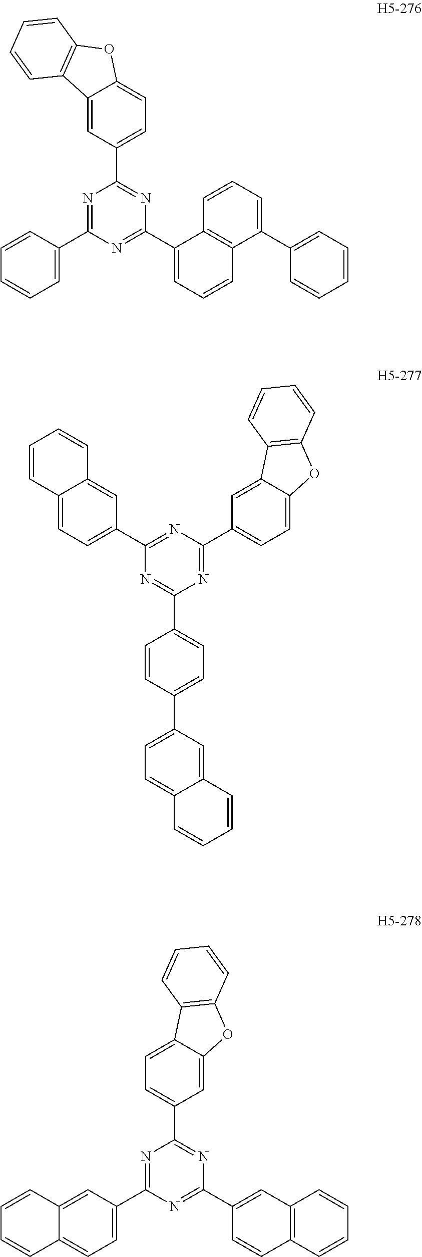 [SMILES: c1ccc(-c2nc(-c3ccc4oc5ccccc5c4c3)nc(-c3cccc4c(-c5ccccc5)cccc34)n2)cc1 ; c1ccc2cc(-c3ccc(-c4nc(-c5ccc6ccccc6c5)nc(-c5ccc6oc7ccccc7c6c5)n4)cc3)ccc2c1 ; c1ccc2cc(-c3nc(-c4ccc5ccccc5c4)nc(-c4ccc5c(c4)oc4ccccc45)n3)ccc2c1]

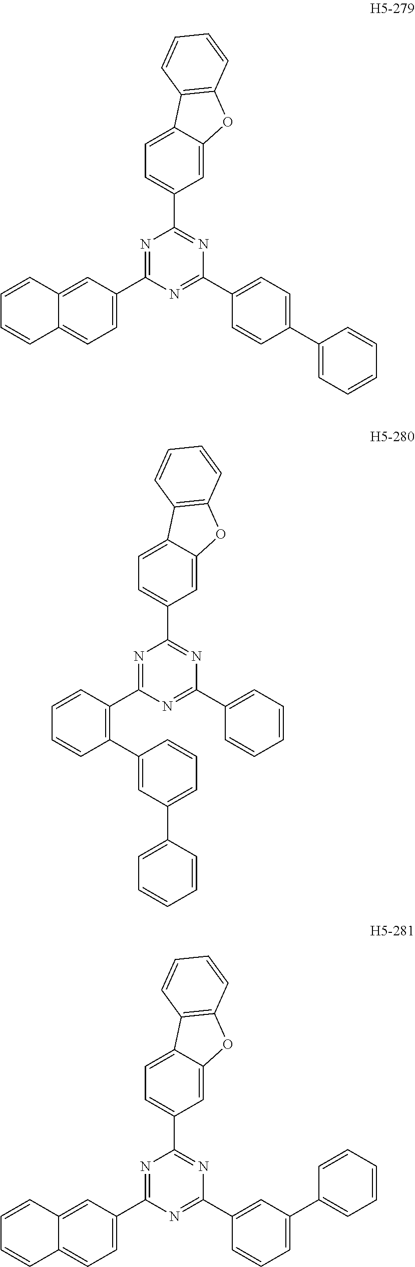 [SMILES: c1ccc(-c2ccc(-c3nc(-c4ccc5ccccc5c4)nc(-c4ccc5c(c4)oc4ccccc45)n3)cc2)cc1 ; c1ccc(-c2cccc(-c3ccccc3-c3nc(-c4ccccc4)nc(-c4ccc5c(c4)oc4ccccc45)n3)c2)cc1 ; c1ccc(-c2cccc(-c3nc(-c4ccc5ccccc5c4)nc(-c4ccc5c(c4)oc4ccccc45)n3)c2)cc1]

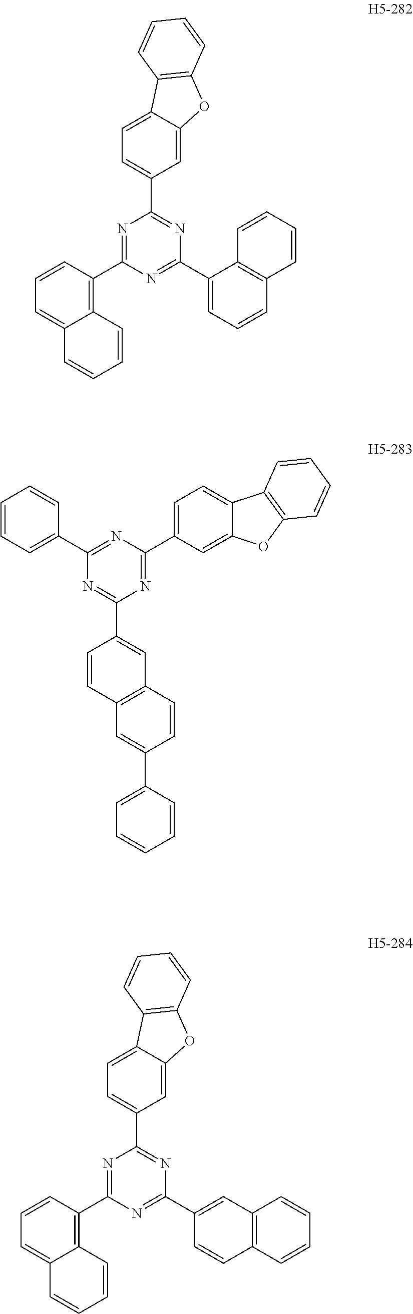 [SMILES: c1ccc2c(-c3nc(-c4ccc5c(c4)oc4ccccc45)nc(-c4cccc5ccccc45)n3)cccc2c1 ; c1ccc(-c2ccc3cc(-c4nc(-c5ccccc5)nc(-c5ccc6c(c5)oc5ccccc56)n4)ccc3c2)cc1 ; c1ccc2cc(-c3nc(-c4ccc5c(c4)oc4ccccc45)nc(-c4cccc5ccccc45)n3)ccc2c1]

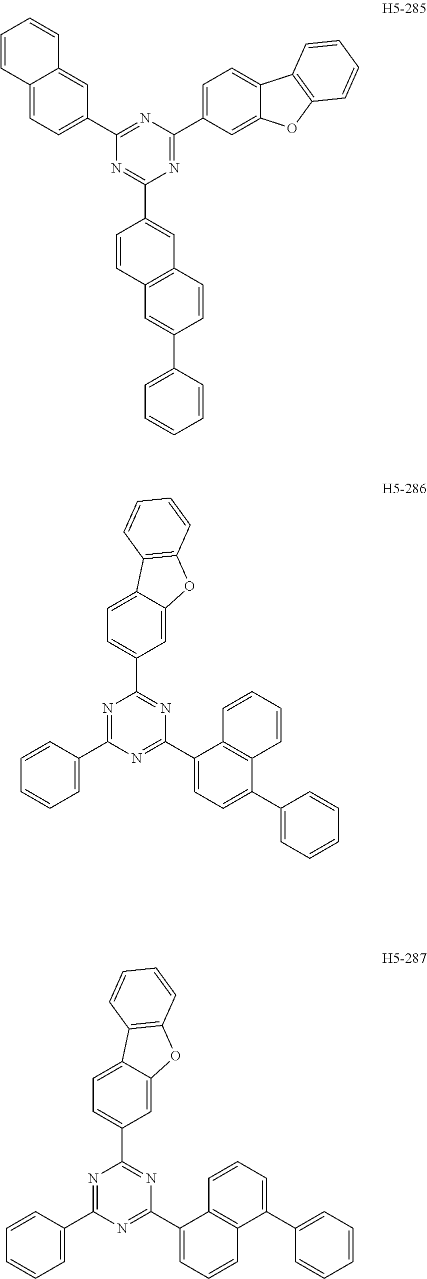 [SMILES: c1ccc(-c2ccc3cc(-c4nc(-c5ccc6ccccc6c5)nc(-c5ccc6c(c5)oc5ccccc56)n4)ccc3c2)cc1 ; c1ccc(-c2nc(-c3ccc4c(c3)oc3ccccc34)nc(-c3ccc(-c4ccccc4)c4ccccc34)n2)cc1 ; c1ccc(-c2nc(-c3ccc4c(c3)oc3ccccc34)nc(-c3cccc4c(-c5ccccc5)cccc34)n2)cc1]

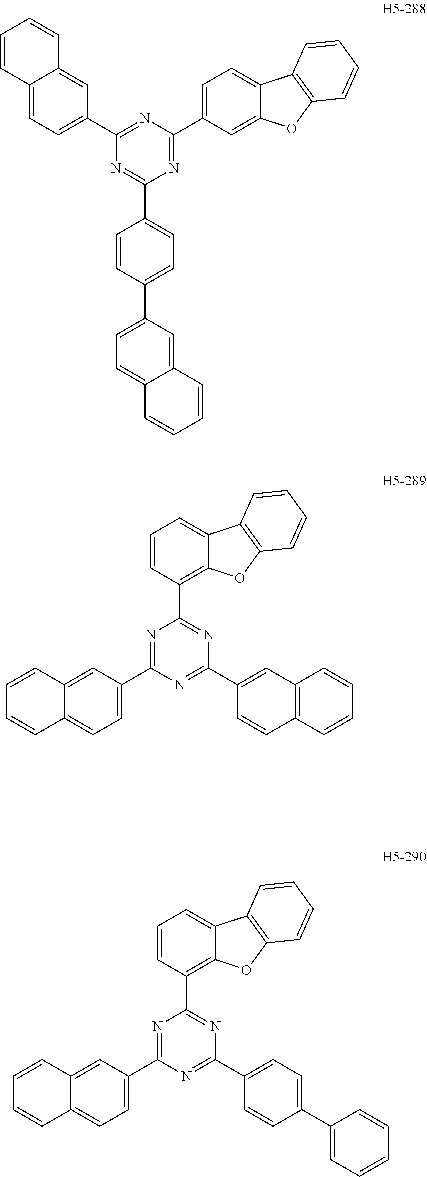 [SMILES: c1ccc2cc(-c3ccc(-c4nc(-c5ccc6ccccc6c5)nc(-c5ccc6c(c5)oc5ccccc56)n4)cc3)ccc2c1 ; c1ccc2cc(-c3nc(-c4ccc5ccccc5c4)nc(-c4cccc5c4oc4ccccc45)n3)ccc2c1 ; c1ccc(-c2ccc(-c3nc(-c4ccc5ccccc5c4)nc(-c4cccc5c4oc4ccccc45)n3)cc2)cc1]

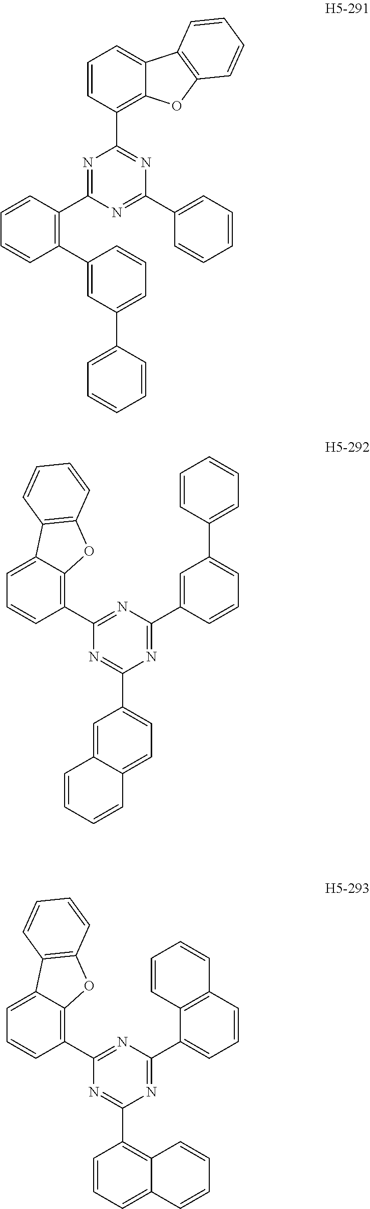 [SMILES: c1ccc(-c2cccc(-c3ccccc3-c3nc(-c4ccccc4)nc(-c4cccc5c4oc4ccccc45)n3)c2)cc1 ; c1ccc(-c2cccc(-c3nc(-c4ccc5ccccc5c4)nc(-c4cccc5c4oc4ccccc45)n3)c2)cc1 ; c1ccc2c(-c3nc(-c4cccc5ccccc45)nc(-c4cccc5c4oc4ccccc45)n3)cccc2c1]

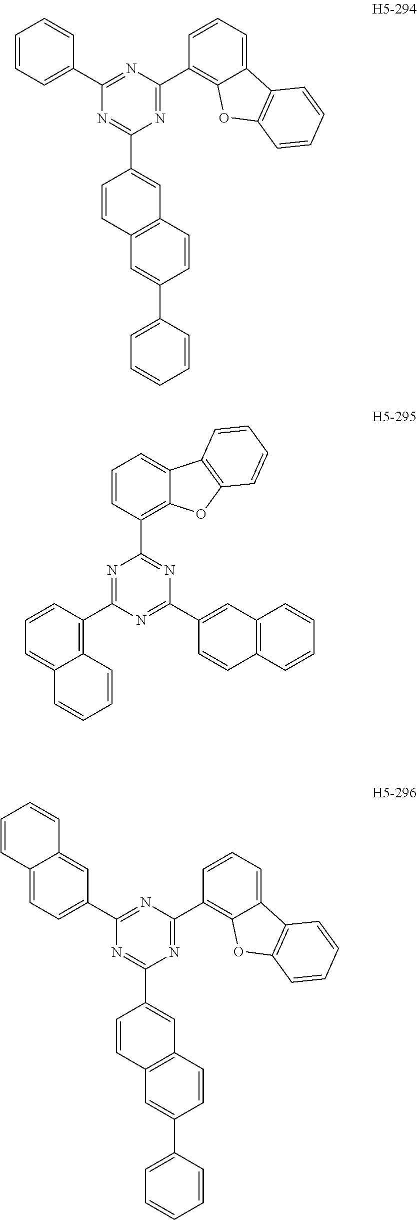 [SMILES: c1ccc(-c2ccc3cc(-c4nc(-c5ccccc5)nc(-c5cccc6c5oc5ccccc56)n4)ccc3c2)cc1 ; c1ccc2cc(-c3nc(-c4cccc5ccccc45)nc(-c4cccc5c4oc4ccccc45)n3)ccc2c1 ; c1ccc(-c2ccc3cc(-c4nc(-c5ccc6ccccc6c5)nc(-c5cccc6c5oc5ccccc56)n4)ccc3c2)cc1]

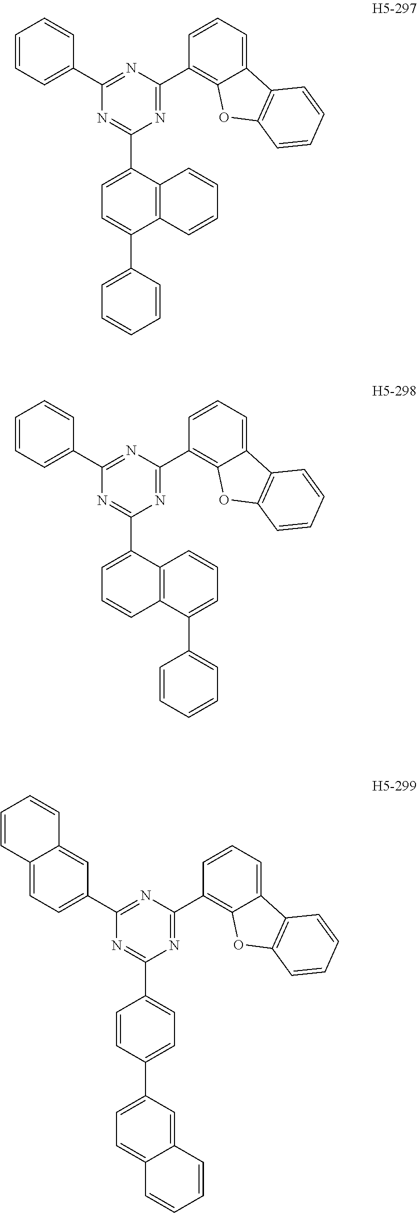 [SMILES: c1ccc(-c2nc(-c3ccc(-c4ccccc4)c4ccccc34)nc(-c3cccc4c3oc3ccccc34)n2)cc1 ; c1ccc(-c2nc(-c3cccc4c(-c5ccccc5)cccc34)nc(-c3cccc4c3oc3ccccc34)n2)cc1 ; c1ccc2cc(-c3ccc(-c4nc(-c5ccc6ccccc6c5)nc(-c5cccc6c5oc5ccccc56)n4)cc3)ccc2c1]

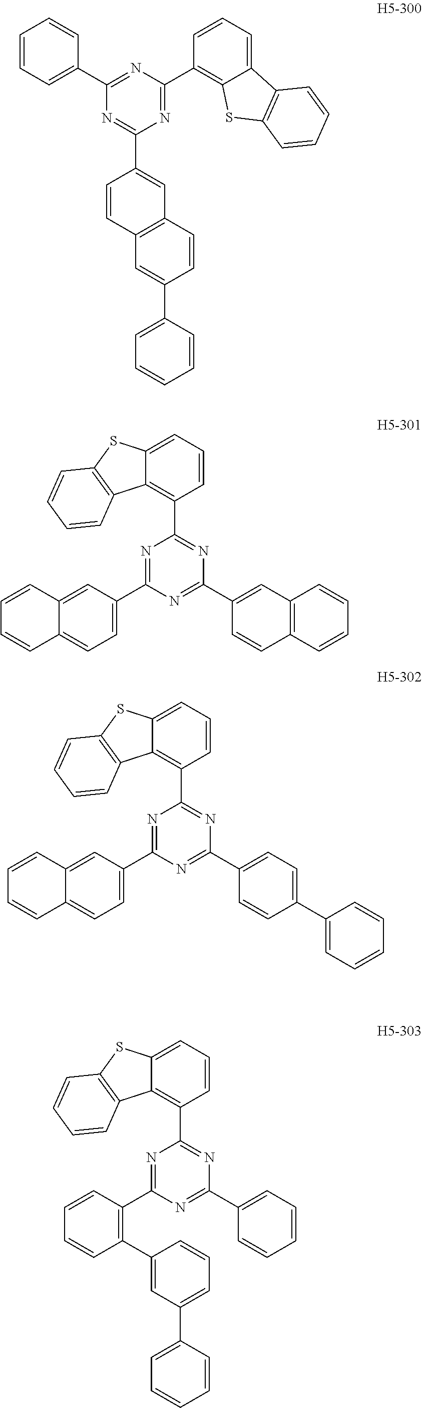 [SMILES: c1ccc(-c2ccc3cc(-c4nc(-c5ccccc5)nc(-c5cccc6c5sc5ccccc56)n4)ccc3c2)cc1 ; c1ccc2cc(-c3nc(-c4ccc5ccccc5c4)nc(-c4cccc5sc6ccccc6c45)n3)ccc2c1 ; c1ccc(-c2ccc(-c3nc(-c4ccc5ccccc5c4)nc(-c4cccc5sc6ccccc6c45)n3)cc2)cc1 ; c1ccc(-c2cccc(-c3ccccc3-c3nc(-c4ccccc4)nc(-c4cccc5sc6ccccc6c45)n3)c2)cc1]

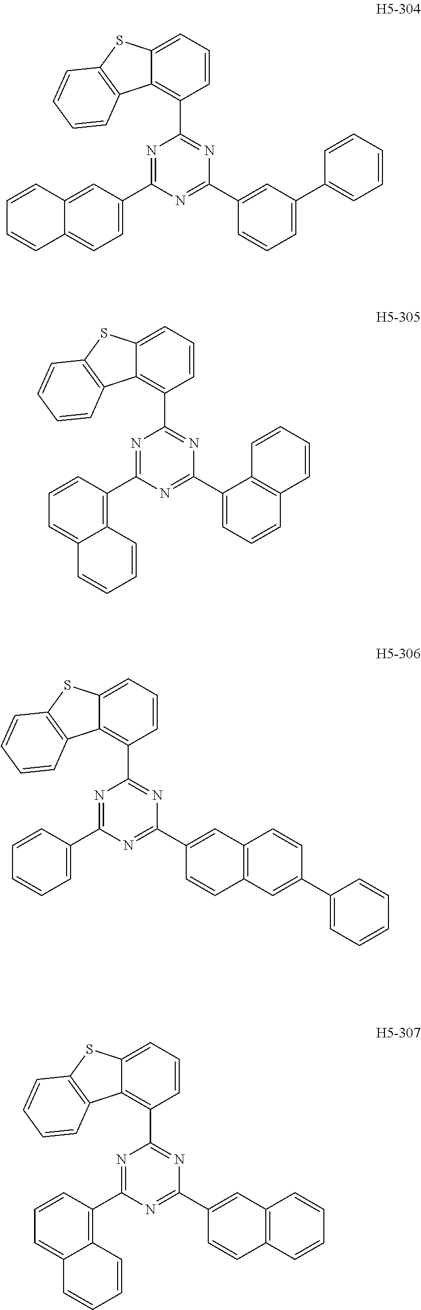 [SMILES: c1ccc(-c2cccc(-c3nc(-c4ccc5ccccc5c4)nc(-c4cccc5sc6ccccc6c45)n3)c2)cc1 ; c1ccc2c(-c3nc(-c4cccc5ccccc45)nc(-c4cccc5sc6ccccc6c45)n3)cccc2c1 ; c1ccc(-c2ccc3cc(-c4nc(-c5ccccc5)nc(-c5cccc6sc7ccccc7c56)n4)ccc3c2)cc1 ; c1ccc2cc(-c3nc(-c4cccc5ccccc45)nc(-c4cccc5sc6ccccc6c45)n3)ccc2c1]

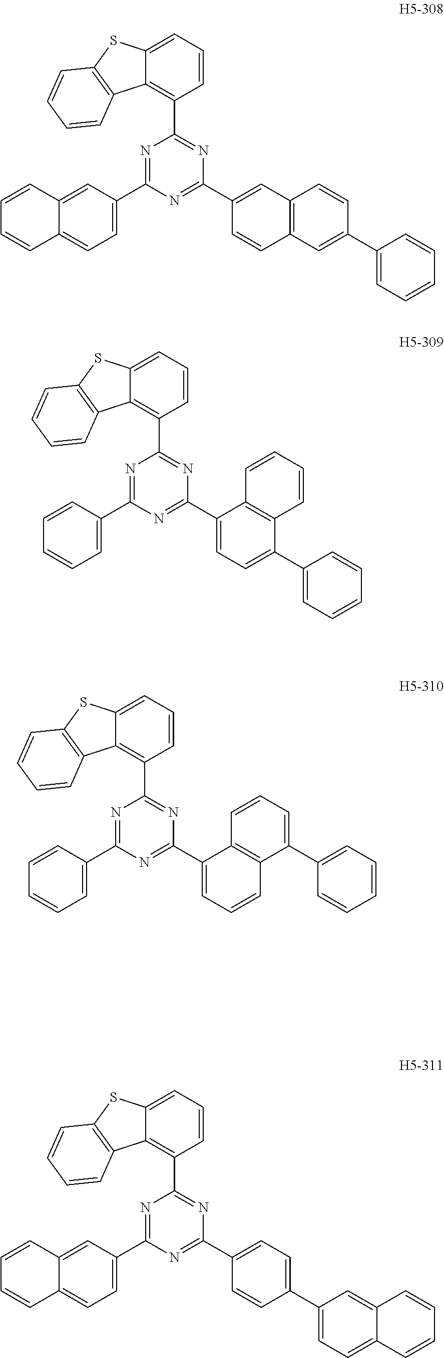 [SMILES: c1ccc(-c2ccc3cc(-c4nc(-c5ccc6ccccc6c5)nc(-c5cccc6sc7ccccc7c56)n4)ccc3c2)cc1 ; c1ccc(-c2nc(-c3ccc(-c4ccccc4)c4ccccc34)nc(-c3cccc4sc5ccccc5c34)n2)cc1 ; c1ccc(-c2nc(-c3cccc4c(-c5ccccc5)cccc34)nc(-c3cccc4sc5ccccc5c34)n2)cc1 ; c1ccc2cc(-c3ccc(-c4nc(-c5ccc6ccccc6c5)nc(-c5cccc6sc7ccccc7c56)n4)cc3)ccc2c1]

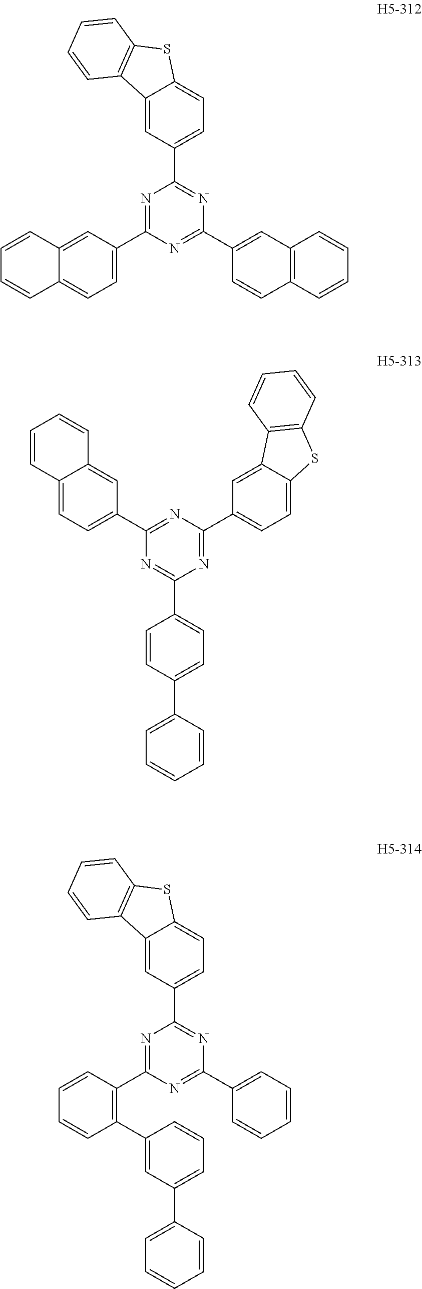 [SMILES: c1ccc2cc(-c3nc(-c4ccc5ccccc5c4)nc(-c4ccc5sc6ccccc6c5c4)n3)ccc2c1 ; c1ccc(-c2ccc(-c3nc(-c4ccc5ccccc5c4)nc(-c4ccc5sc6ccccc6c5c4)n3)cc2)cc1 ; c1ccc(-c2cccc(-c3ccccc3-c3nc(-c4ccccc4)nc(-c4ccc5sc6ccccc6c5c4)n3)c2)cc1]

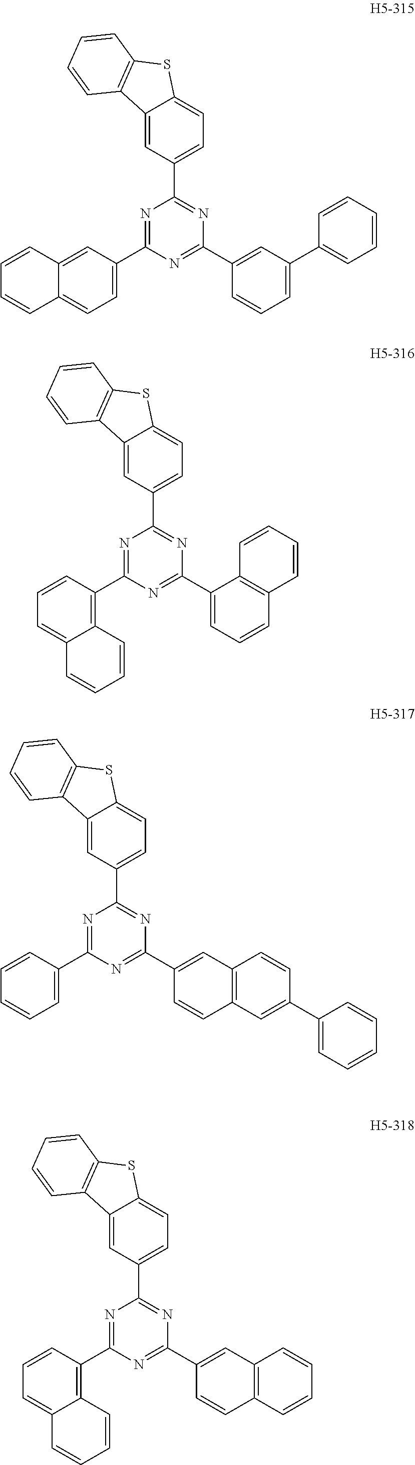 [SMILES: c1ccc(-c2cccc(-c3nc(-c4ccc5ccccc5c4)nc(-c4ccc5sc6ccccc6c5c4)n3)c2)cc1 ; c1ccc2c(-c3nc(-c4ccc5sc6ccccc6c5c4)nc(-c4cccc5ccccc45)n3)cccc2c1 ; c1ccc(-c2ccc3cc(-c4nc(-c5ccccc5)nc(-c5ccc6sc7ccccc7c6c5)n4)ccc3c2)cc1 ; c1ccc2cc(-c3nc(-c4ccc5sc6ccccc6c5c4)nc(-c4cccc5ccccc45)n3)ccc2c1]

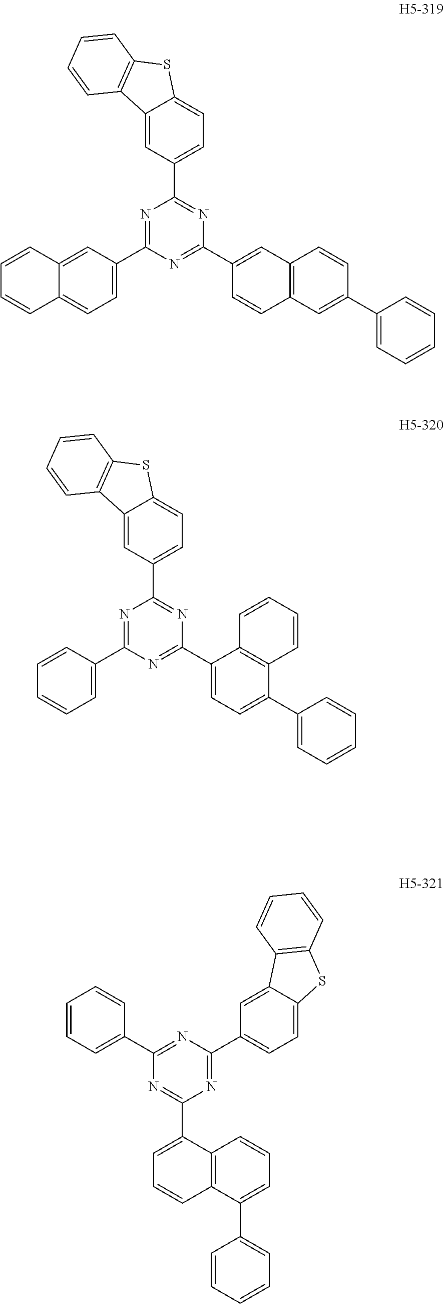 [SMILES: c1ccc(-c2ccc3cc(-c4nc(-c5ccc6ccccc6c5)nc(-c5ccc6sc7ccccc7c6c5)n4)ccc3c2)cc1 ; c1ccc(-c2nc(-c3ccc4sc5ccccc5c4c3)nc(-c3ccc(-c4ccccc4)c4ccccc34)n2)cc1 ; c1ccc(-c2nc(-c3ccc4sc5ccccc5c4c3)nc(-c3cccc4c(-c5ccccc5)cccc34)n2)cc1]

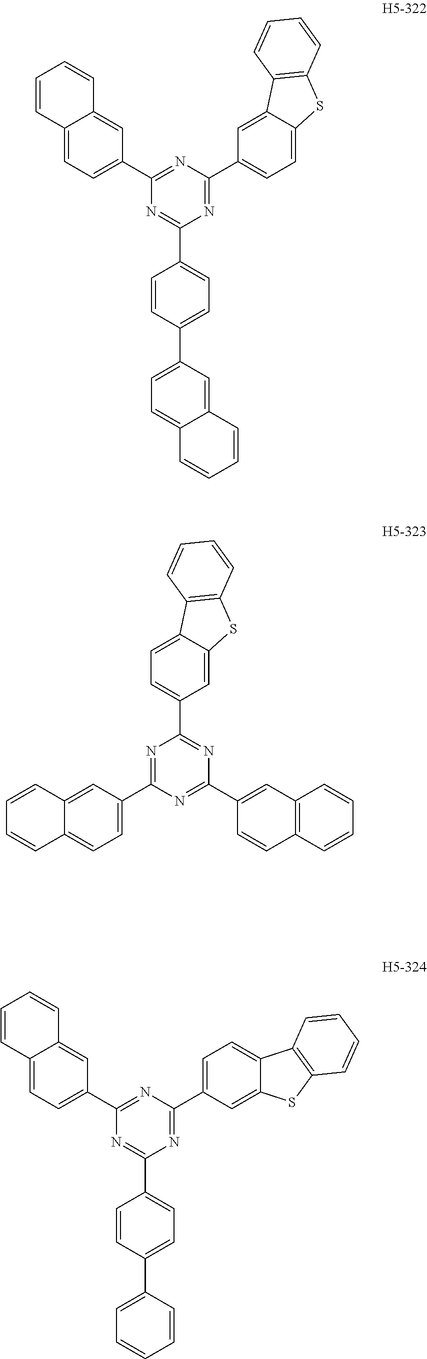 [SMILES: c1ccc2cc(-c3ccc(-c4nc(-c5ccc6ccccc6c5)nc(-c5ccc6sc7ccccc7c6c5)n4)cc3)ccc2c1 ; c1ccc2cc(-c3nc(-c4ccc5ccccc5c4)nc(-c4ccc5c(c4)sc4ccccc45)n3)ccc2c1 ; c1ccc(-c2ccc(-c3nc(-c4ccc5ccccc5c4)nc(-c4ccc5c(c4)sc4ccccc45)n3)cc2)cc1]

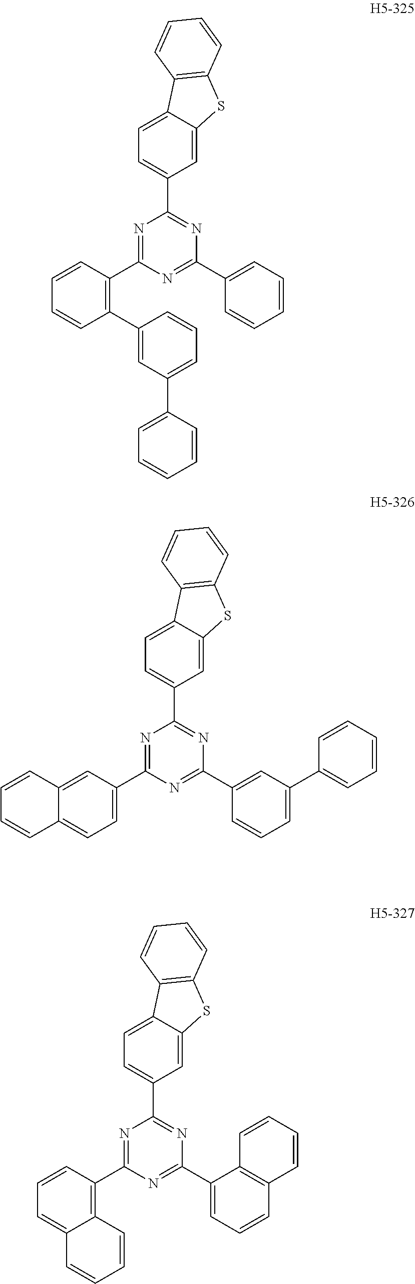 [SMILES: c1ccc(-c2cccc(-c3ccccc3-c3nc(-c4ccccc4)nc(-c4ccc5c(c4)sc4ccccc45)n3)c2)cc1 ; c1ccc(-c2cccc(-c3nc(-c4ccc5ccccc5c4)nc(-c4ccc5c(c4)sc4ccccc45)n3)c2)cc1 ; c1ccc2c(-c3nc(-c4ccc5c(c4)sc4ccccc45)nc(-c4cccc5ccccc45)n3)cccc2c1]

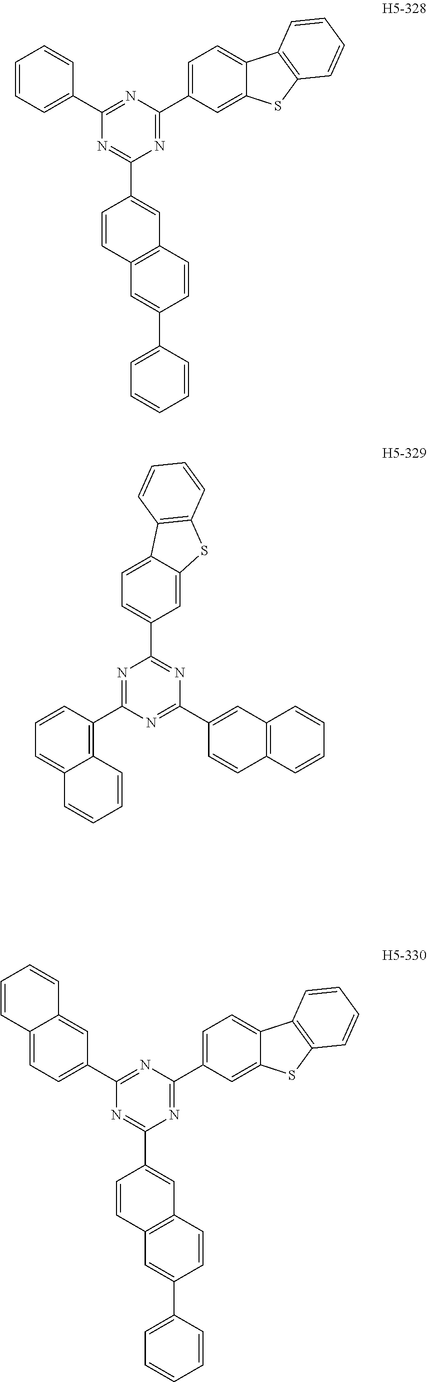 [SMILES: c1ccc(-c2ccc3cc(-c4nc(-c5ccccc5)nc(-c5ccc6c(c5)sc5ccccc56)n4)ccc3c2)cc1 ; c1ccc2cc(-c3nc(-c4ccc5c(c4)sc4ccccc45)nc(-c4cccc5ccccc45)n3)ccc2c1 ; c1ccc(-c2ccc3cc(-c4nc(-c5ccc6ccccc6c5)nc(-c5ccc6c(c5)sc5ccccc56)n4)ccc3c2)cc1]

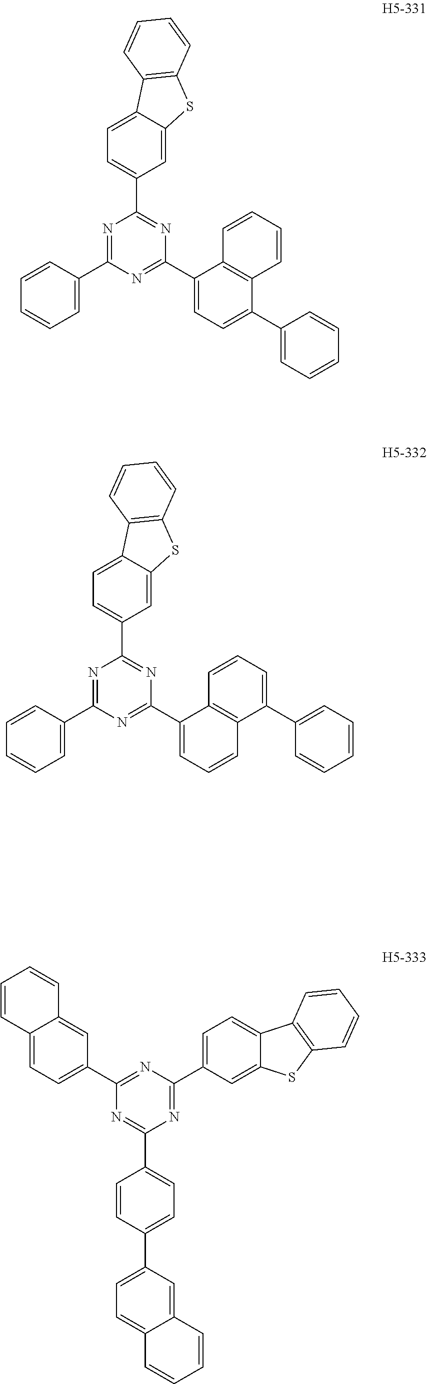 [SMILES: c1ccc(-c2nc(-c3ccc4c(c3)sc3ccccc34)nc(-c3ccc(-c4ccccc4)c4ccccc34)n2)cc1 ; c1ccc(-c2nc(-c3ccc4c(c3)sc3ccccc34)nc(-c3cccc4c(-c5ccccc5)cccc34)n2)cc1 ; c1ccc2cc(-c3ccc(-c4nc(-c5ccc6ccccc6c5)nc(-c5ccc6c(c5)sc5ccccc56)n4)cc3)ccc2c1]

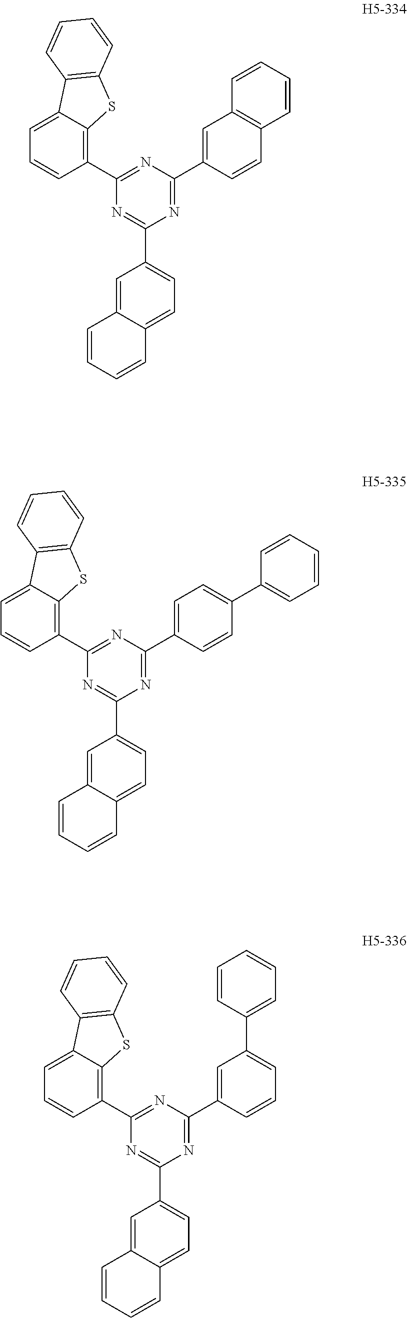 [SMILES: c1ccc2cc(-c3nc(-c4ccc5ccccc5c4)nc(-c4cccc5c4sc4ccccc45)n3)ccc2c1 ; c1ccc(-c2ccc(-c3nc(-c4ccc5ccccc5c4)nc(-c4cccc5c4sc4ccccc45)n3)cc2)cc1 ; c1ccc(-c2cccc(-c3nc(-c4ccc5ccccc5c4)nc(-c4cccc5c4sc4ccccc45)n3)c2)cc1]

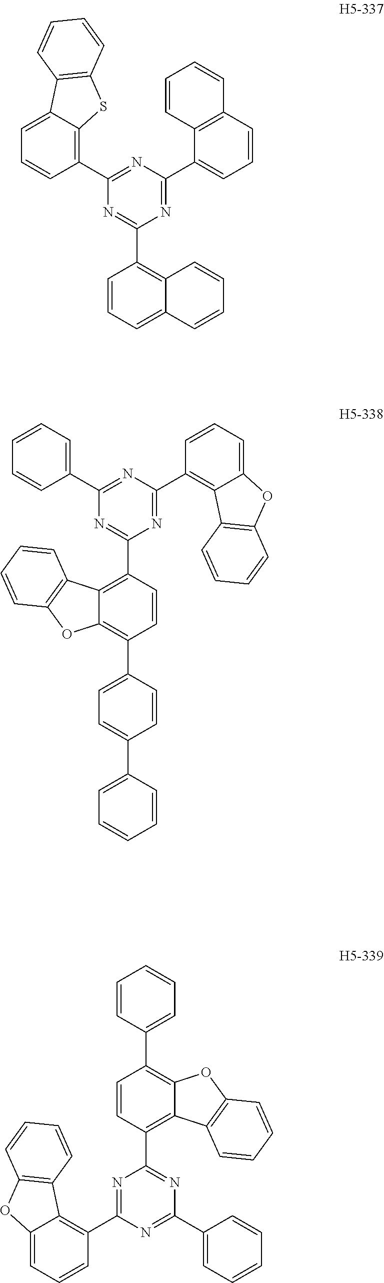 [SMILES: c1ccc2c(-c3nc(-c4cccc5ccccc45)nc(-c4cccc5c4sc4ccccc45)n3)cccc2c1 ; c1ccc(-c2ccc(-c3ccc(-c4nc(-c5ccccc5)nc(-c5cccc6oc7ccccc7c56)n4)c4c3oc3ccccc34)cc2)cc1 ; c1ccc(-c2nc(-c3cccc4oc5ccccc5c34)nc(-c3ccc(-c4ccccc4)c4oc5ccccc5c34)n2)cc1]

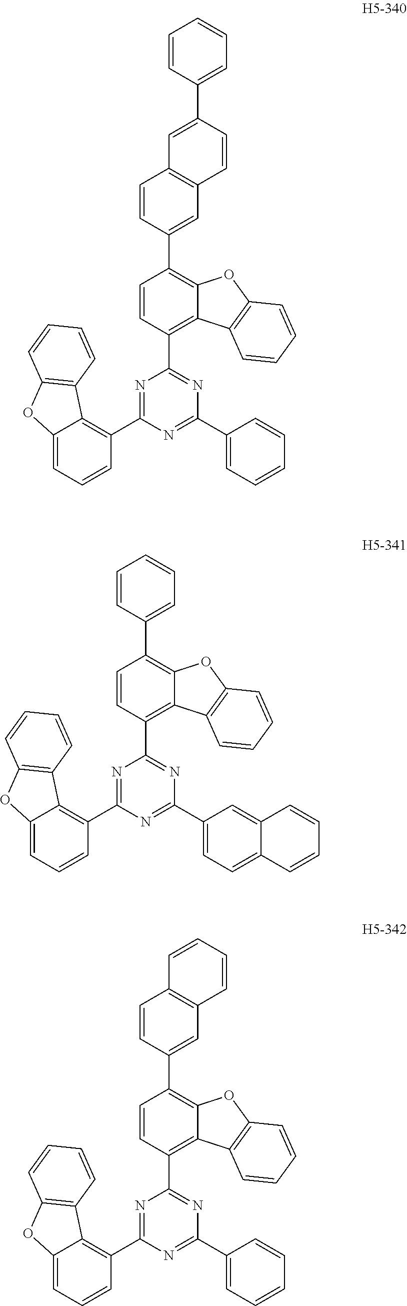 [SMILES: c1ccc(-c2ccc3cc(-c4ccc(-c5nc(-c6ccccc6)nc(-c6cccc7oc8ccccc8c67)n5)c5c4oc4ccccc45)ccc3c2)cc1 ; c1ccc(-c2ccc(-c3nc(-c4ccc5ccccc5c4)nc(-c4cccc5oc6ccccc6c45)n3)c3c2oc2ccccc23)cc1 ; c1ccc(-c2nc(-c3cccc4oc5ccccc5c34)nc(-c3ccc(-c4ccc5ccccc5c4)c4oc5ccccc5c34)n2)cc1]

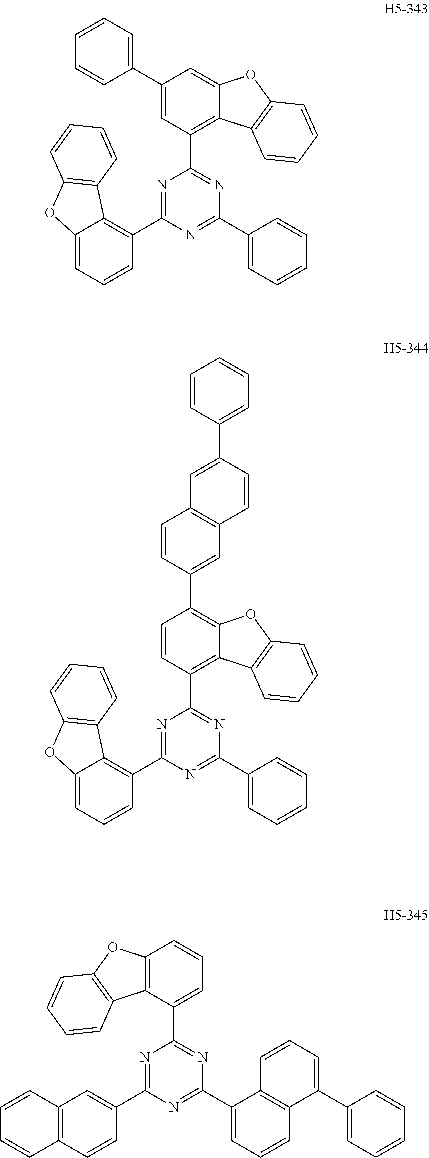 [SMILES: c1ccc(-c2cc(-c3nc(-c4ccccc4)nc(-c4cccc5oc6ccccc6c45)n3)c3c(c2)oc2ccccc23)cc1 ; c1ccc(-c2ccc3cc(-c4ccc(-c5nc(-c6ccccc6)nc(-c6cccc7oc8ccccc8c67)n5)c5c4oc4ccccc45)ccc3c2)cc1 ; c1ccc(-c2cccc3c(-c4nc(-c5ccc6ccccc6c5)nc(-c5cccc6oc7ccccc7c56)n4)cccc23)cc1]

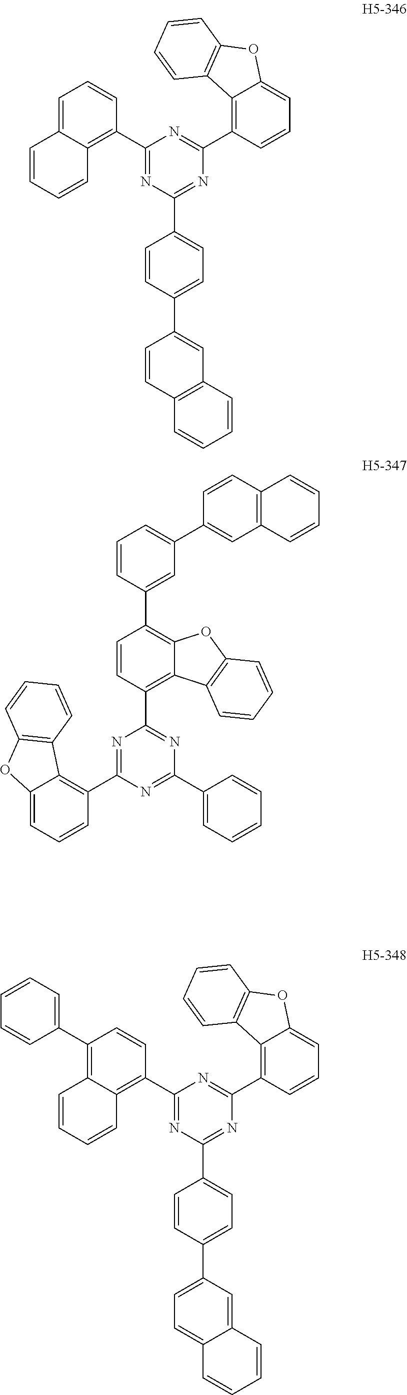

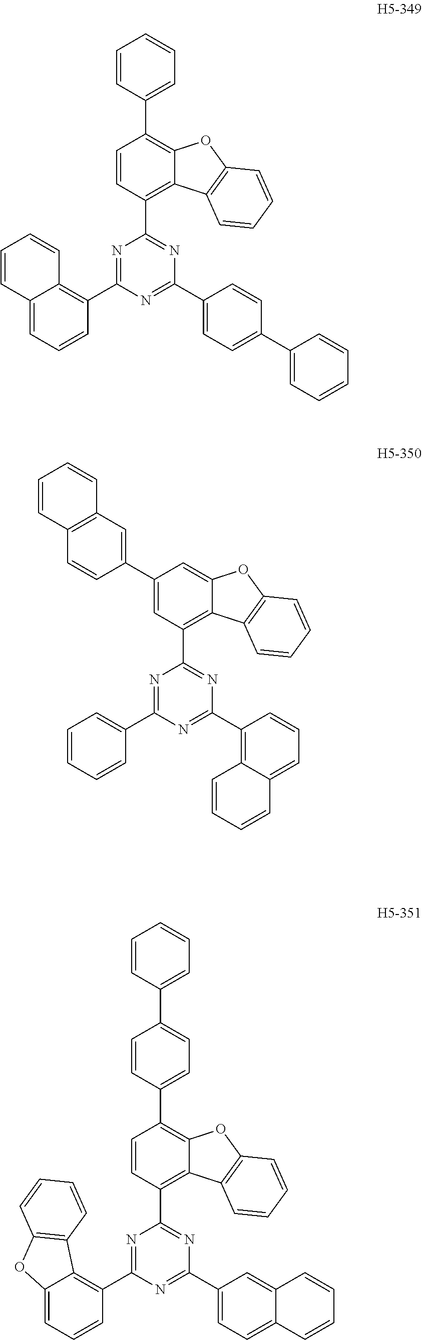 [SMILES: c1ccc(-c2ccc(-c3nc(-c4cccc5ccccc45)nc(-c4ccc(-c5ccccc5)c5oc6ccccc6c45)n3)cc2)cc1 ; c1ccc(-c2nc(-c3cccc4ccccc34)nc(-c3cc(-c4ccc5ccccc5c4)cc4oc5ccccc5c34)n2)cc1 ; c1ccc(-c2ccc(-c3ccc(-c4nc(-c5ccc6ccccc6c5)nc(-c5cccc6oc7ccccc7c56)n4)c4c3oc3ccccc34)cc2)cc1]

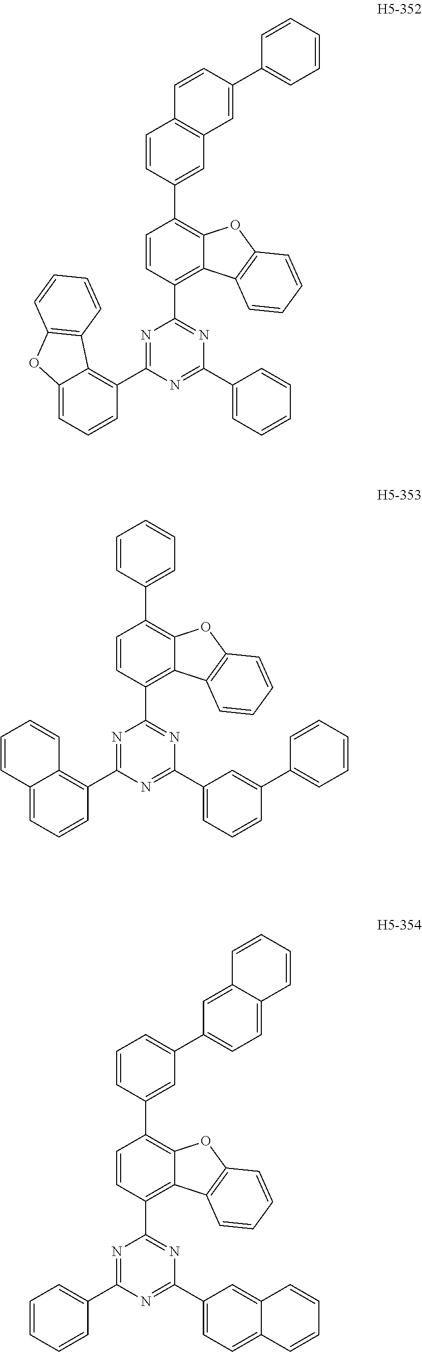 [SMILES: c1ccc(-c2ccc3ccc(-c4ccc(-c5nc(-c6ccccc6)nc(-c6cccc7oc8ccccc8c67)n5)c5c4oc4ccccc45)cc3c2)cc1 ; c1ccc(-c2cccc(-c3nc(-c4cccc5ccccc45)nc(-c4ccc(-c5ccccc5)c5oc6ccccc6c45)n3)c2)cc1 ; c1ccc(-c2nc(-c3ccc4ccccc4c3)nc(-c3ccc(-c4cccc(-c5ccc6ccccc6c5)c4)c4oc5ccccc5c34)n2)cc1]

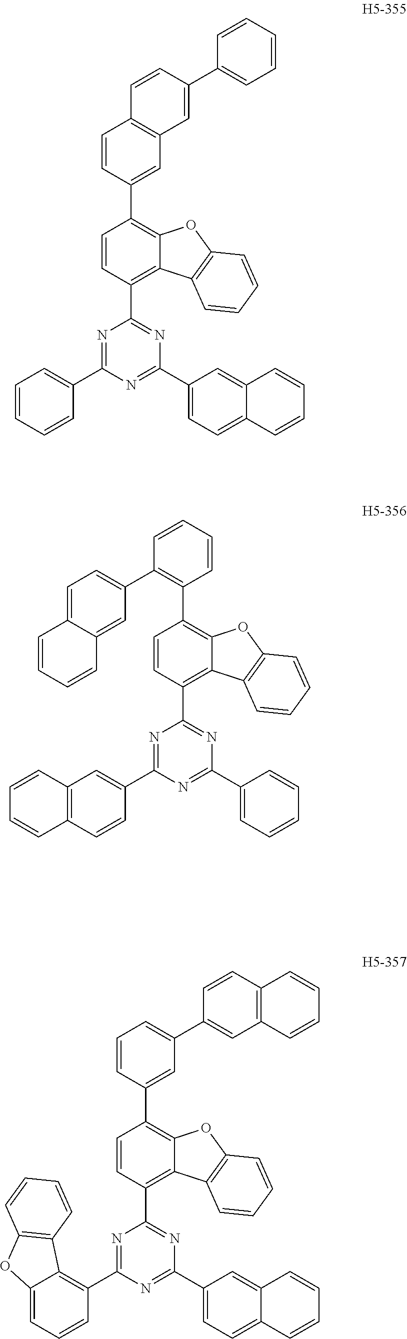 [SMILES: c1ccc(-c2ccc3ccc(-c4ccc(-c5nc(-c6ccccc6)nc(-c6ccc7ccccc7c6)n5)c5c4oc4ccccc45)cc3c2)cc1 ; c1ccc(-c2nc(-c3ccc4ccccc4c3)nc(-c3ccc(-c4ccccc4-c4ccc5ccccc5c4)c4oc5ccccc5c34)n2)cc1 ; c1cc(-c2ccc3ccccc3c2)cc(-c2ccc(-c3nc(-c4ccc5ccccc5c4)nc(-c4cccc5oc6ccccc6c45)n3)c3c2oc2ccccc23)c1]

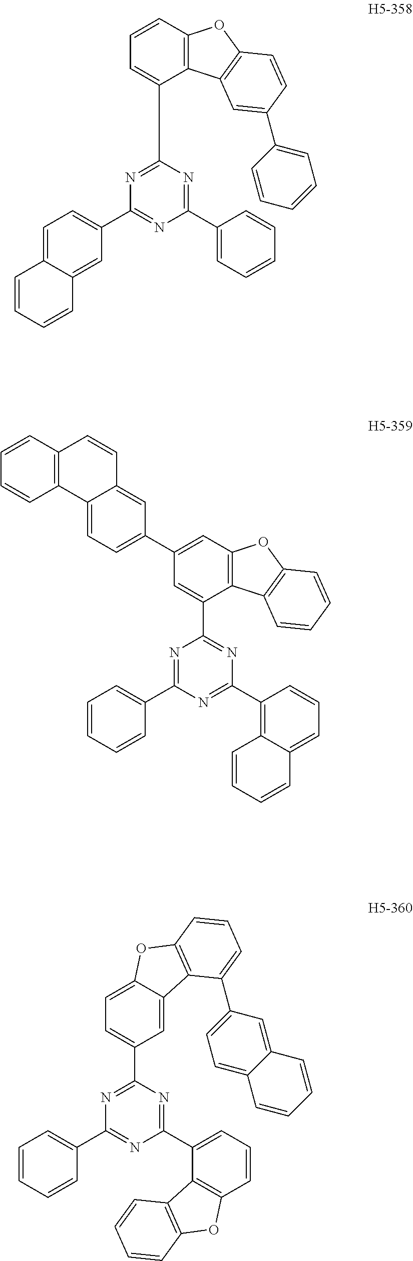 [SMILES: c1ccc(-c2ccc3oc4cccc(-c5nc(-c6ccccc6)nc(-c6ccc7ccccc7c6)n5)c4c3c2)cc1 ; c1ccc(-c2nc(-c3cccc4ccccc34)nc(-c3cc(-c4ccc5c(ccc6ccccc65)c4)cc4oc5ccccc5c34)n2)cc1 ; c1ccc(-c2nc(-c3ccc4oc5cccc(-c6ccc7ccccc7c6)c5c4c3)nc(-c3cccc4oc5ccccc5c34)n2)cc1]

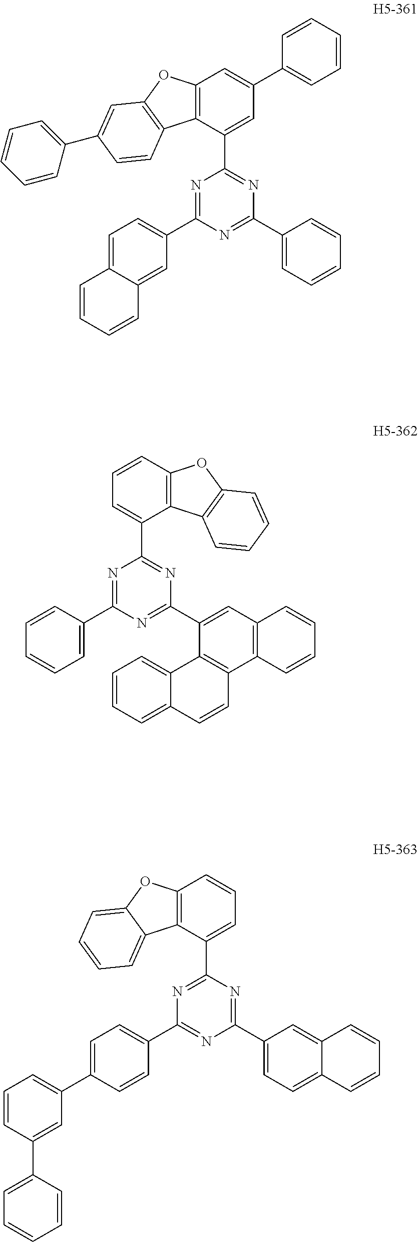 [SMILES: c1ccc(-c2ccc3c(c2)oc2cc(-c4ccccc4)cc(-c4nc(-c5ccccc5)nc(-c5ccc6ccccc6c5)n4)c23)cc1 ; c1ccc(-c2nc(-c3cccc4oc5ccccc5c34)nc(-c3cc4ccccc4c4ccc5ccccc5c34)n2)cc1 ; c1ccc(-c2cccc(-c3ccc(-c4nc(-c5ccc6ccccc6c5)nc(-c5cccc6oc7ccccc7c56)n4)cc3)c2)cc1]

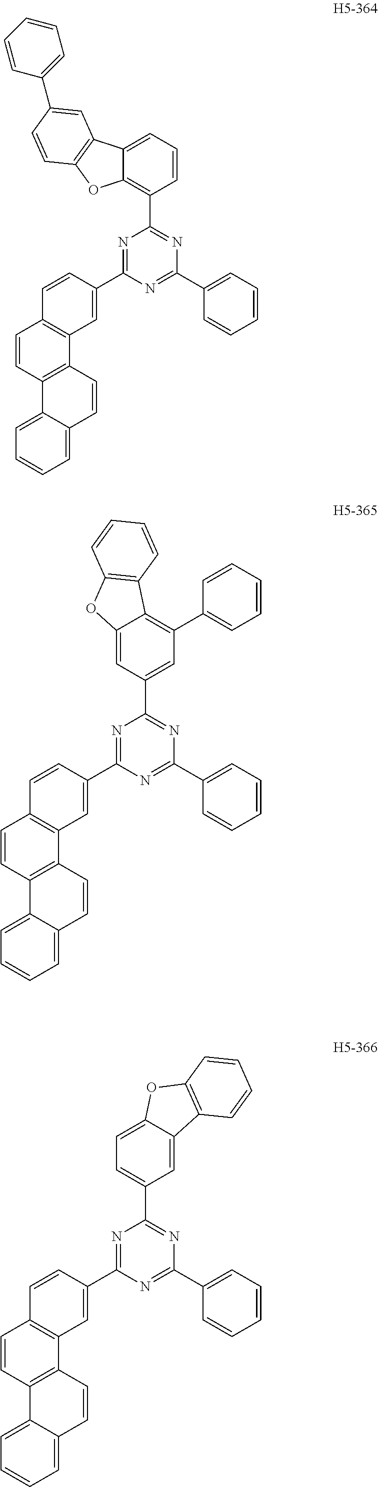 [SMILES: c1ccc(-c2ccc3oc4c(-c5nc(-c6ccccc6)nc(-c6ccc7ccc8c9ccccc9ccc8c7c6)n5)cccc4c3c2)cc1 ; c1ccc(-c2nc(-c3cc(-c4ccccc4)c4c(c3)oc3ccccc34)nc(-c3ccc4ccc5c6ccccc6ccc5c4c3)n2)cc1 ; c1ccc(-c2nc(-c3ccc4ccc5c6ccccc6ccc5c4c3)nc(-c3ccc4oc5ccccc5c4c3)n2)cc1]

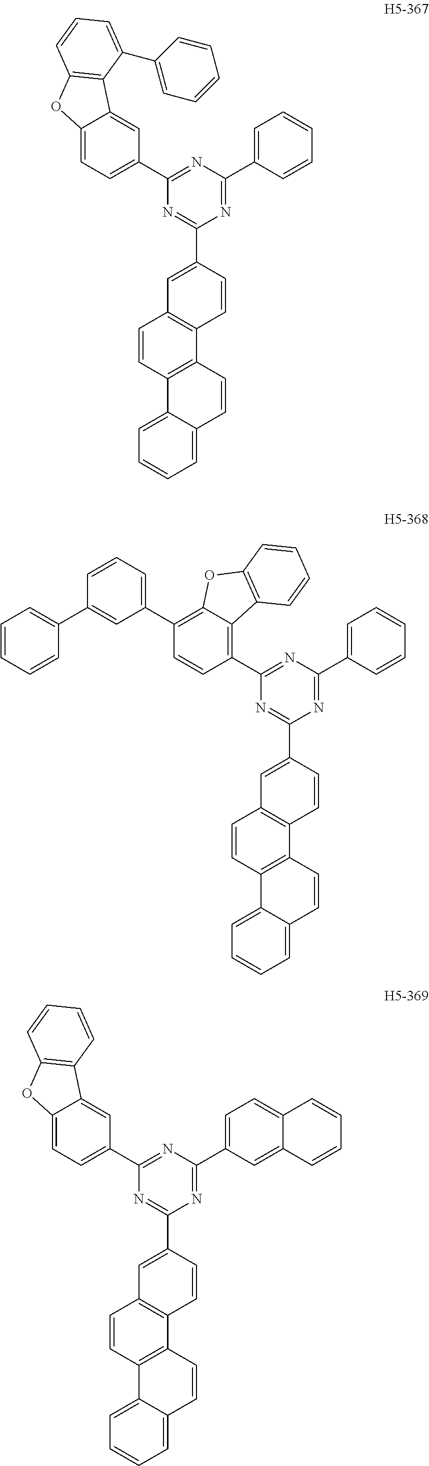 [SMILES: c1ccc(-c2nc(-c3ccc4c(ccc5c6ccccc6ccc45)c3)nc(-c3ccc4oc5cccc(-c6ccccc6)c5c4c3)n2)cc1 ; c1ccc(-c2cccc(-c3ccc(-c4nc(-c5ccccc5)nc(-c5ccc6c(ccc7c8ccccc8ccc67)c5)n4)c4c3oc3ccccc34)c2)cc1 ; c1ccc2cc(-c3nc(-c4ccc5c(ccc6c7ccccc7ccc56)c4)nc(-c4ccc5oc6ccccc6c5c4)n3)ccc2c1]

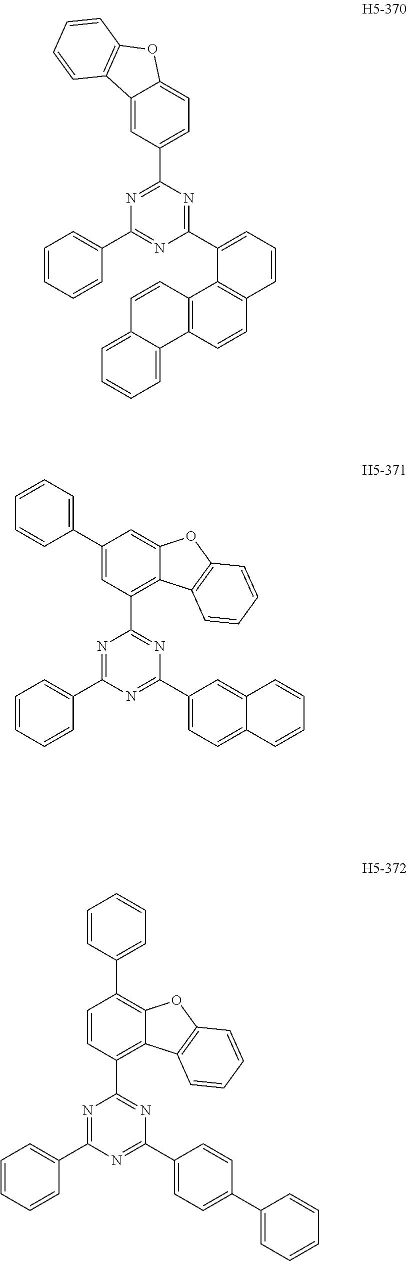 [SMILES: c1ccc(-c2nc(-c3ccc4oc5ccccc5c4c3)nc(-c3cccc4ccc5c6ccccc6ccc5c34)n2)cc1 ; c1ccc(-c2cc(-c3nc(-c4ccccc4)nc(-c4ccc5ccccc5c4)n3)c3c(c2)oc2ccccc23)cc1 ; c1ccc(-c2ccc(-c3nc(-c4ccccc4)nc(-c4ccc(-c5ccccc5)c5oc6ccccc6c45)n3)cc2)cc1]

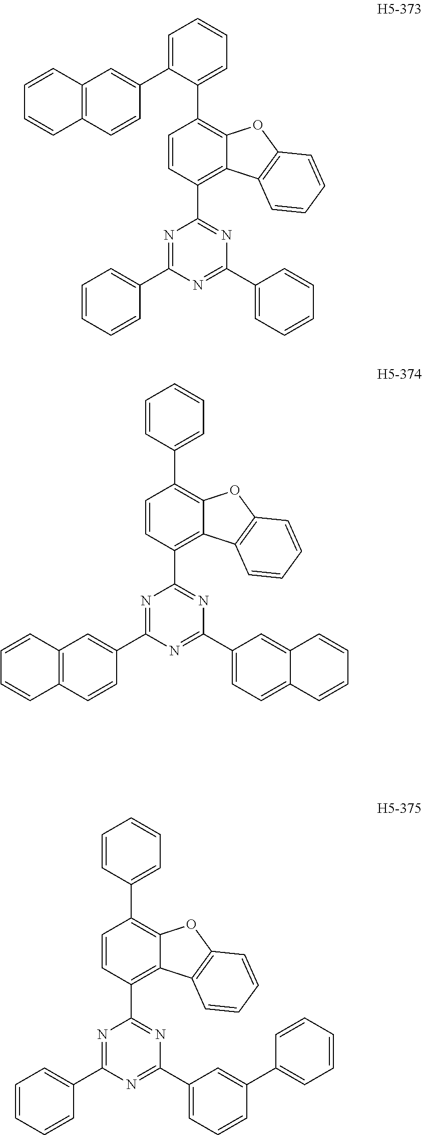 [SMILES: c1ccc(-c2nc(-c3ccccc3)nc(-c3ccc(-c4ccccc4-c4ccc5ccccc5c4)c4oc5ccccc5c34)n2)cc1 ; c1ccc(-c2ccc(-c3nc(-c4ccc5ccccc5c4)nc(-c4ccc5ccccc5c4)n3)c3c2oc2ccccc23)cc1 ; c1ccc(-c2cccc(-c3nc(-c4ccccc4)nc(-c4ccc(-c5ccccc5)c5oc6ccccc6c45)n3)c2)cc1]

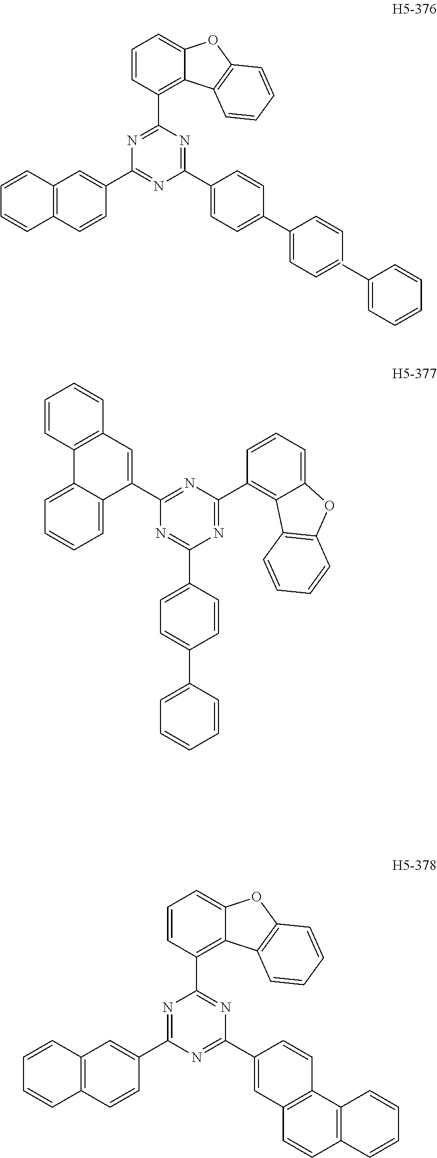

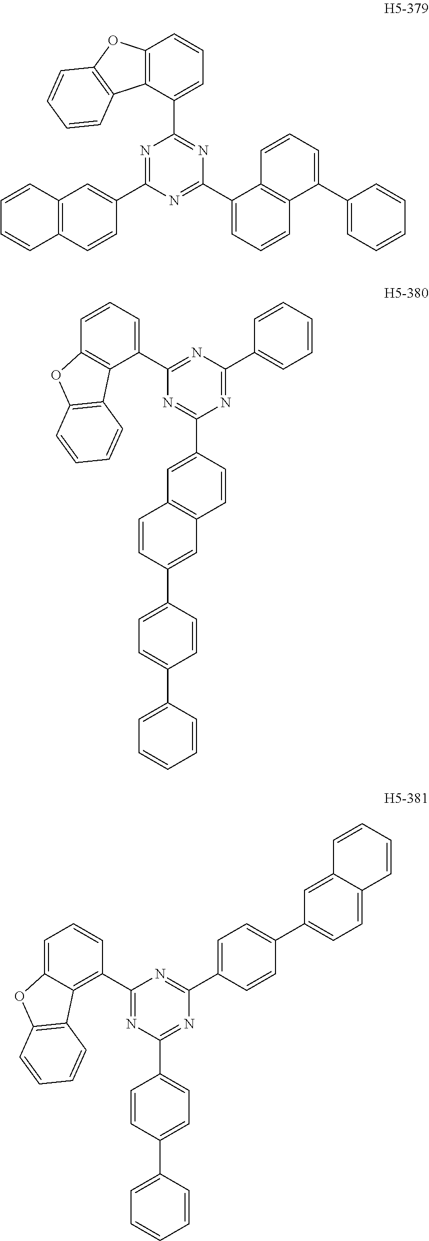 [SMILES: c1ccc(-c2cccc3c(-c4nc(-c5ccc6ccccc6c5)nc(-c5cccc6oc7ccccc7c56)n4)cccc23)cc1 ; c1ccc(-c2ccc(-c3ccc4cc(-c5nc(-c6ccccc6)nc(-c6cccc7oc8ccccc8c67)n5)ccc4c3)cc2)cc1 ; c1ccc(-c2ccc(-c3nc(-c4ccc(-c5ccc6ccccc6c5)cc4)nc(-c4cccc5oc6ccccc6c45)n3)cc2)cc1]

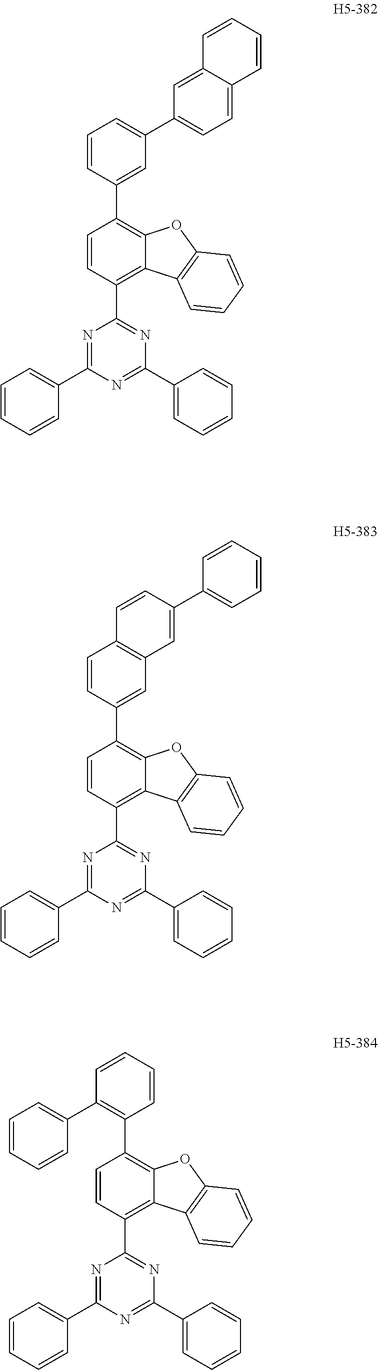 [SMILES: c1ccc(-c2nc(-c3ccccc3)nc(-c3ccc(-c4cccc(-c5ccc6ccccc6c5)c4)c4oc5ccccc5c34)n2)cc1 ; c1ccc(-c2ccc3ccc(-c4ccc(-c5nc(-c6ccccc6)nc(-c6ccccc6)n5)c5c4oc4ccccc45)cc3c2)cc1 ; c1ccc(-c2nc(-c3ccccc3)nc(-c3ccc(-c4ccccc4-c4ccccc4)c4oc5ccccc5c34)n2)cc1]

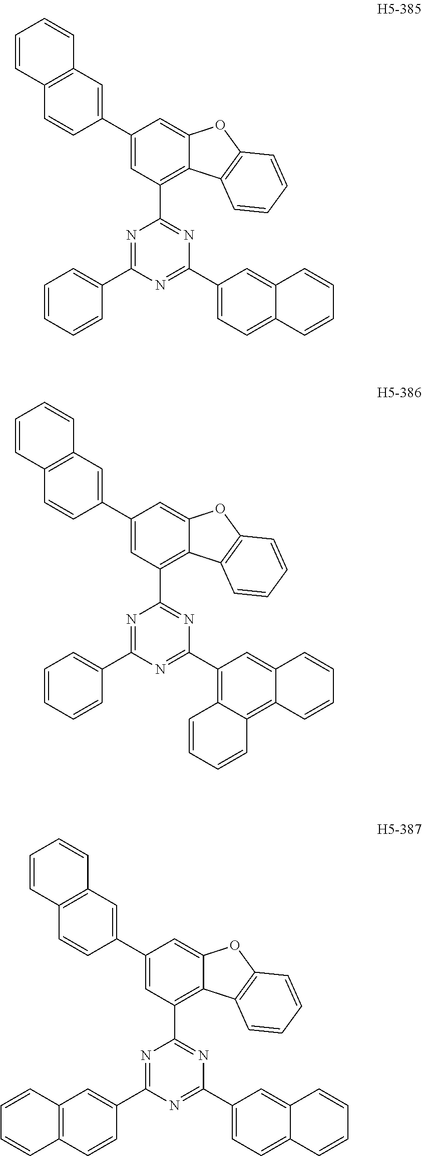 [SMILES: c1ccc(-c2nc(-c3ccc4ccccc4c3)nc(-c3cc(-c4ccc5ccccc5c4)cc4oc5ccccc5c34)n2)cc1 ; c1ccc(-c2nc(-c3cc4ccccc4c4ccccc34)nc(-c3cc(-c4ccc5ccccc5c4)cc4oc5ccccc5c34)n2)cc1 ; c1ccc2cc(-c3cc(-c4nc(-c5ccc6ccccc6c5)nc(-c5ccc6ccccc6c5)n4)c4c(c3)oc3ccccc34)ccc2c1]

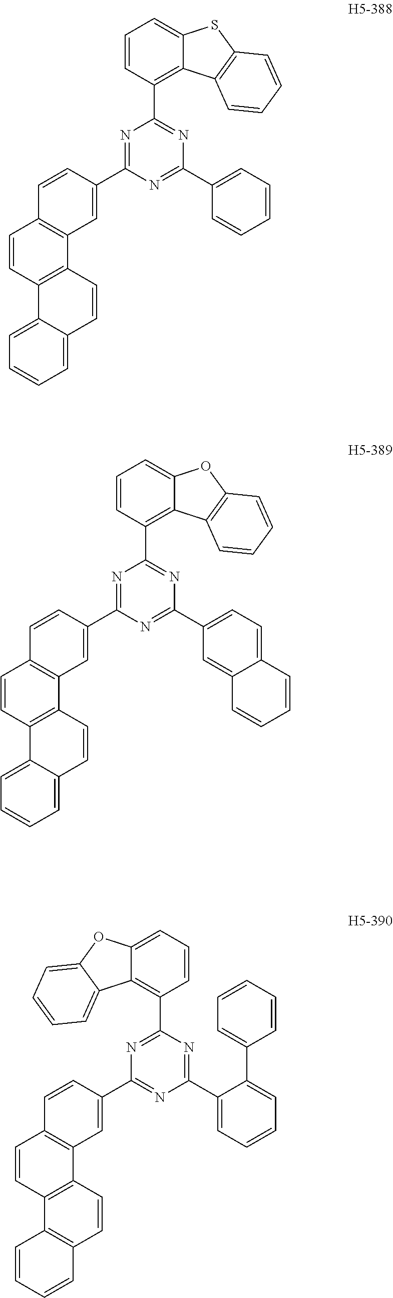 [SMILES: c1ccc(-c2nc(-c3ccc4ccc5c6ccccc6ccc5c4c3)nc(-c3cccc4sc5ccccc5c34)n2)cc1 ; c1ccc2cc(-c3nc(-c4ccc5ccc6c7ccccc7ccc6c5c4)nc(-c4cccc5oc6ccccc6c45)n3)ccc2c1 ; c1ccc(-c2ccccc2-c2nc(-c3ccc4ccc5c6ccccc6ccc5c4c3)nc(-c3cccc4oc5ccccc5c34)n2)cc1]

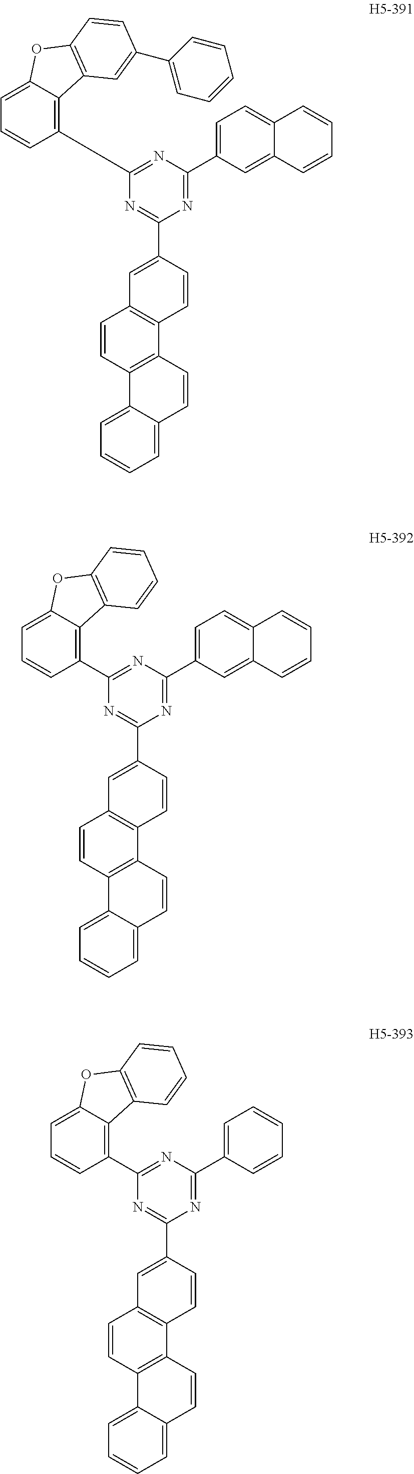 [SMILES: c1ccc(-c2ccc3oc4cccc(-c5nc(-c6ccc7ccccc7c6)nc(-c6ccc7c(ccc8c9ccccc9ccc78)c6)n5)c4c3c2)cc1 ; c1ccc2cc(-c3nc(-c4ccc5c(ccc6c7ccccc7ccc56)c4)nc(-c4cccc5oc6ccccc6c45)n3)ccc2c1 ; c1ccc(-c2nc(-c3ccc4c(ccc5c6ccccc6ccc45)c3)nc(-c3cccc4oc5ccccc5c34)n2)cc1]

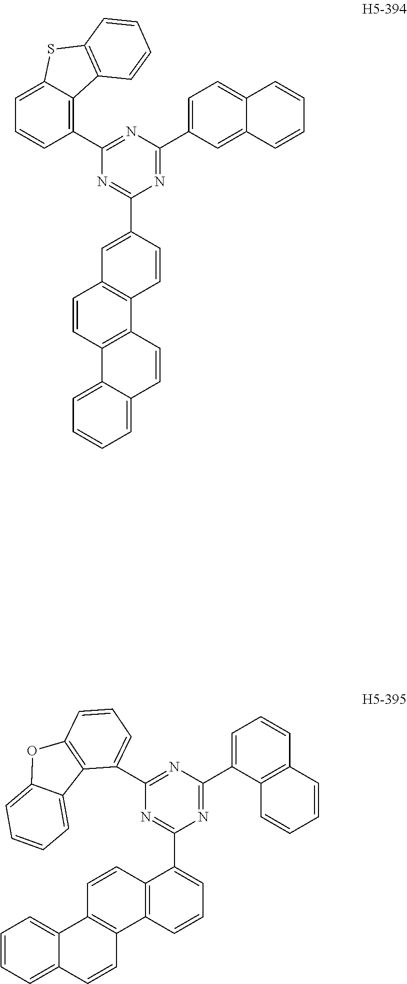 [SMILES: c1ccc2cc(-c3nc(-c4ccc5c(ccc6c7ccccc7ccc56)c4)nc(-c4cccc5sc6ccccc6c45)n3)ccc2c1 ; c1ccc2c(-c3nc(-c4cccc5c4ccc4c6ccccc6ccc54)nc(-c4cccc5oc6ccccc6c45)n3)cccc2c1]

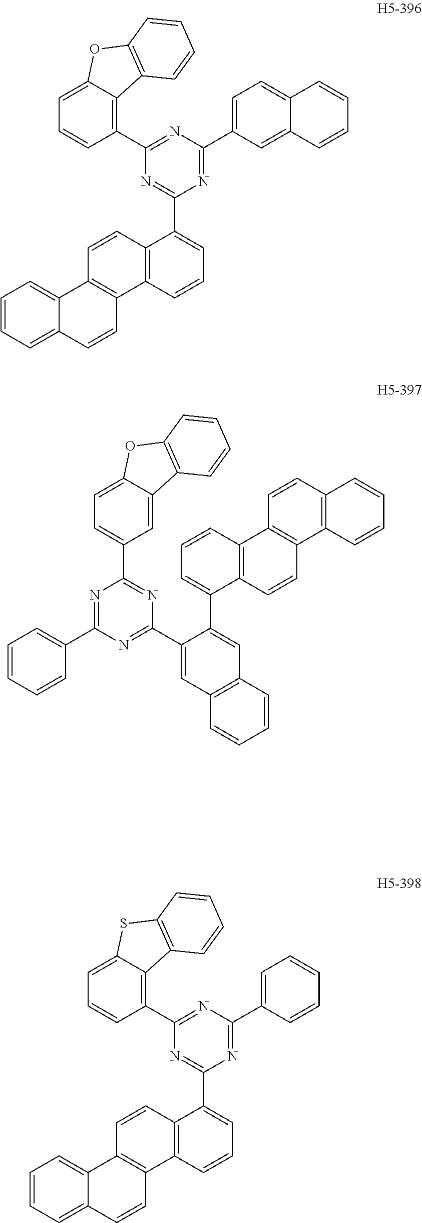 [SMILES: c1ccc2cc(-c3nc(-c4cccc5c4ccc4c6ccccc6ccc54)nc(-c4cccc5oc6ccccc6c45)n3)ccc2c1 ; c1ccc(-c2nc(-c3ccc4oc5ccccc5c4c3)nc(-c3cc4ccccc4cc3-c3cccc4c3ccc3c5ccccc5ccc43)n2)cc1 ; c1ccc(-c2nc(-c3cccc4c3ccc3c5ccccc5ccc43)nc(-c3cccc4sc5ccccc5c34)n2)cc1]

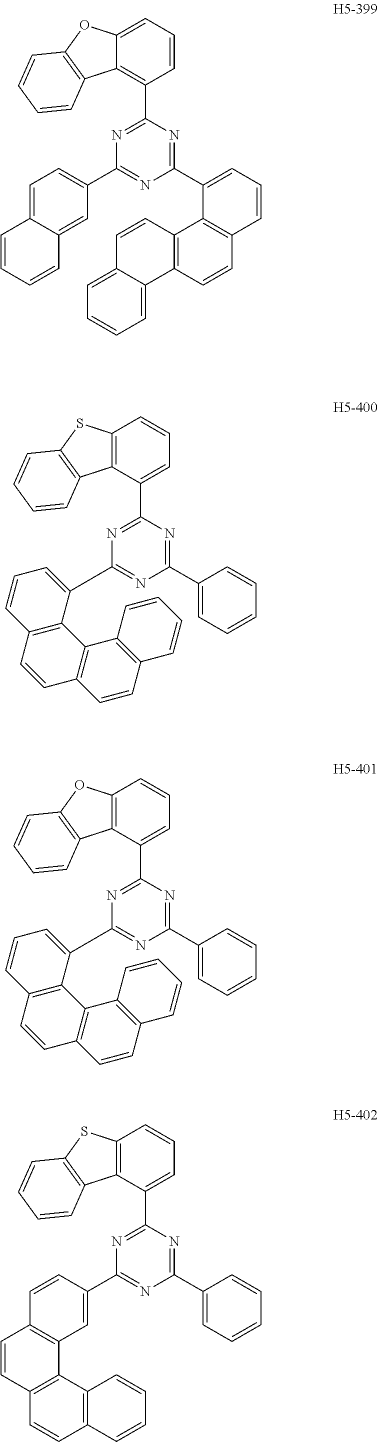 [SMILES: c1ccc2cc(-c3nc(-c4cccc5ccc6c7ccccc7ccc6c45)nc(-c4cccc5oc6ccccc6c45)n3)ccc2c1 ; c1ccc(-c2nc(-c3cccc4sc5ccccc5c34)nc(-c3cccc4ccc5ccc6ccccc6c5c34)n2)cc1 ; c1ccc(-c2nc(-c3cccc4oc5ccccc5c34)nc(-c3cccc4ccc5ccc6ccccc6c5c34)n2)cc1 ; c1ccc(-c2nc(-c3ccc4ccc5ccc6ccccc6c5c4c3)nc(-c3cccc4sc5ccccc5c34)n2)cc1]

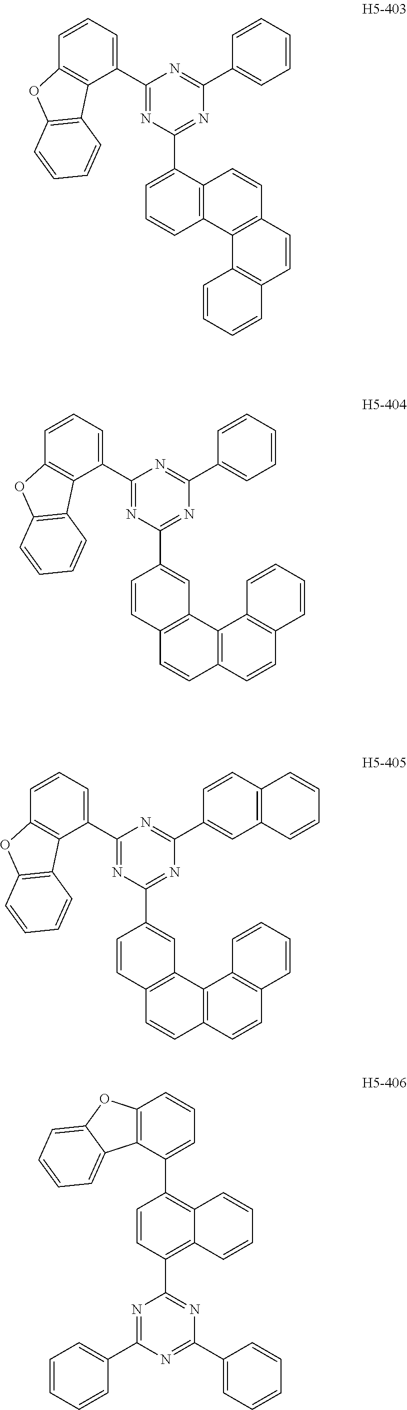 [SMILES: c1ccc(-c2nc(-c3cccc4c3ccc3ccc5ccccc5c34)nc(-c3cccc4oc5ccccc5c34)n2)cc1 ; c1ccc(-c2nc(-c3ccc4ccc5ccc6ccccc6c5c4c3)nc(-c3cccc4oc5ccccc5c34)n2)cc1 ; c1ccc2cc(-c3nc(-c4ccc5ccc6ccc7ccccc7c6c5c4)nc(-c4cccc5oc6ccccc6c45)n3)ccc2c1 ; c1ccc(-c2nc(-c3ccccc3)nc(-c3ccc(-c4cccc5oc6ccccc6c45)c4ccccc34)n2)cc1]

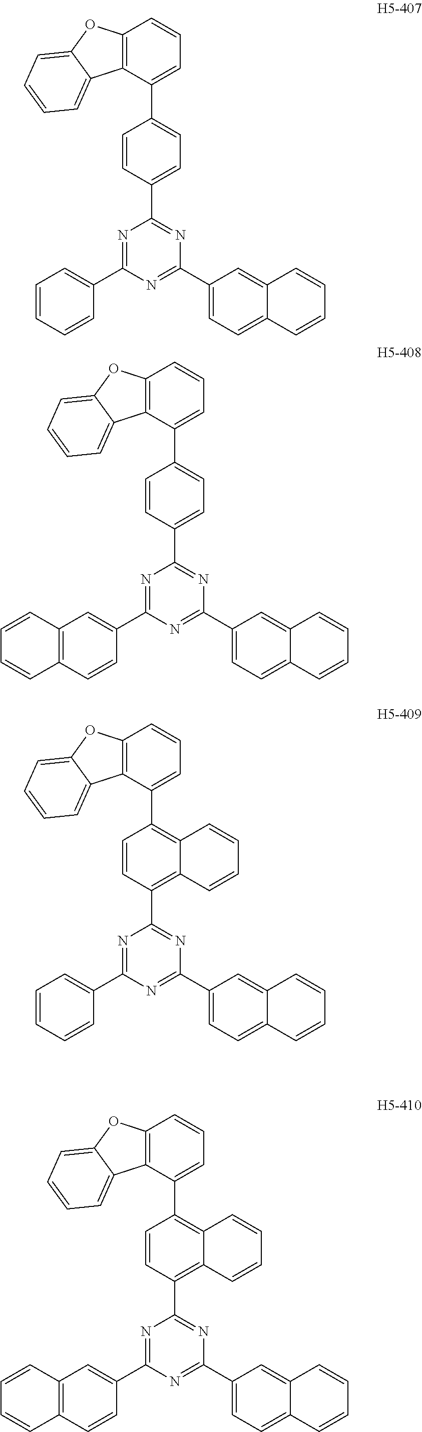 [SMILES: c1ccc(-c2nc(-c3ccc(-c4cccc5oc6ccccc6c45)cc3)nc(-c3ccc4ccccc4c3)n2)cc1 ; c1ccc2cc(-c3nc(-c4ccc(-c5cccc6oc7ccccc7c56)cc4)nc(-c4ccc5ccccc5c4)n3)ccc2c1 ; c1ccc(-c2nc(-c3ccc4ccccc4c3)nc(-c3ccc(-c4cccc5oc6ccccc6c45)c4ccccc34)n2)cc1 ; c1ccc2cc(-c3nc(-c4ccc5ccccc5c4)nc(-c4ccc(-c5cccc6oc7ccccc7c56)c5ccccc45)n3)ccc2c1]

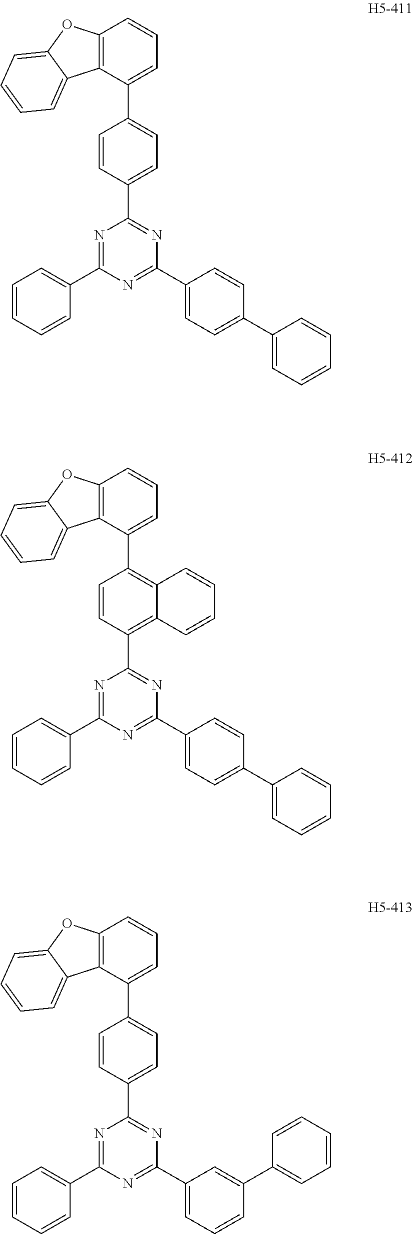 [SMILES: c1ccc(-c2ccc(-c3nc(-c4ccccc4)nc(-c4ccc(-c5cccc6oc7ccccc7c56)cc4)n3)cc2)cc1 ; c1ccc(-c2ccc(-c3nc(-c4ccccc4)nc(-c4ccc(-c5cccc6oc7ccccc7c56)c5ccccc45)n3)cc2)cc1 ; c1ccc(-c2cccc(-c3nc(-c4ccccc4)nc(-c4ccc(-c5cccc6oc7ccccc7c56)cc4)n3)c2)cc1]

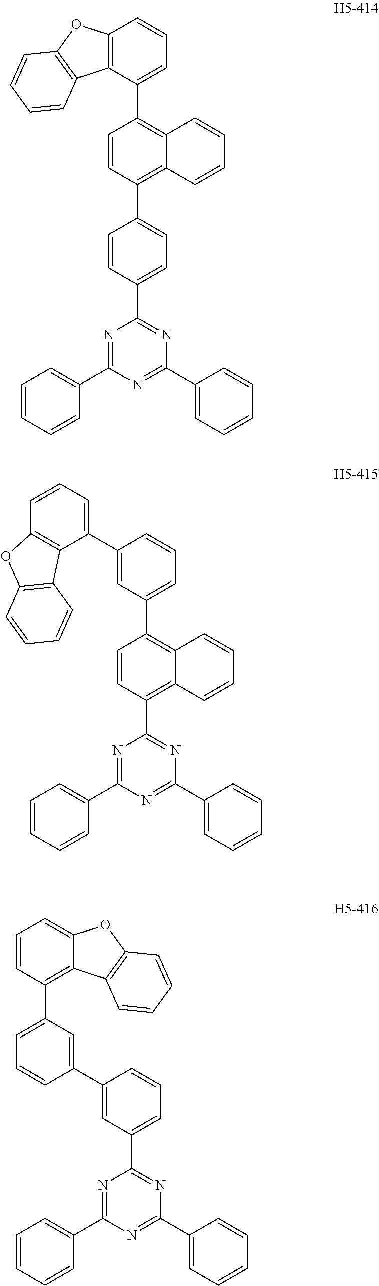 [SMILES: c1ccc(-c2nc(-c3ccccc3)nc(-c3ccc(-c4ccc(-c5cccc6oc7ccccc7c56)c5ccccc45)cc3)n2)cc1 ; c1ccc(-c2nc(-c3ccccc3)nc(-c3ccc(-c4cccc(-c5cccc6oc7ccccc7c56)c4)c4ccccc34)n2)cc1 ; c1ccc(-c2nc(-c3ccccc3)nc(-c3cccc(-c4cccc(-c5cccc6oc7ccccc7c56)c4)c3)n2)cc1]

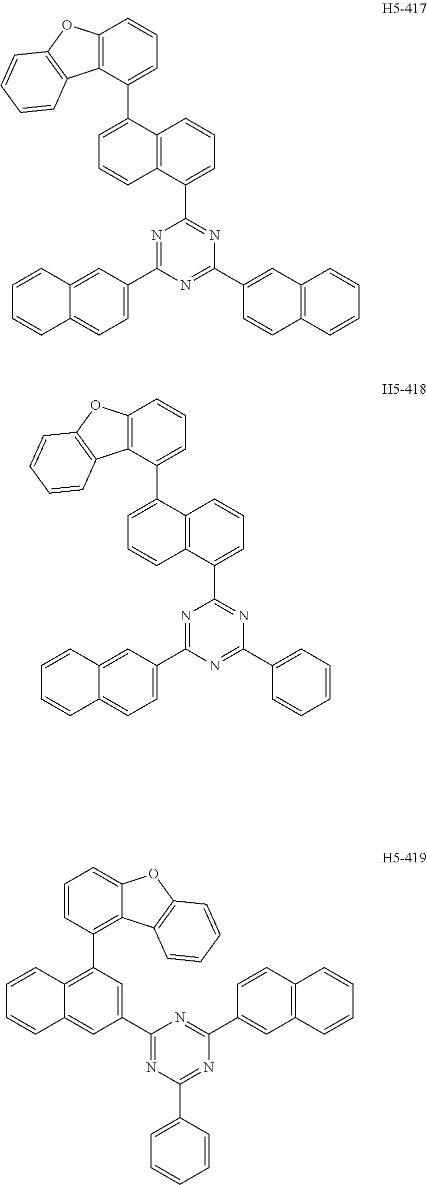 [SMILES: c1ccc2cc(-c3nc(-c4ccc5ccccc5c4)nc(-c4cccc5c(-c6cccc7oc8ccccc8c67)cccc45)n3)ccc2c1 ; c1ccc(-c2nc(-c3ccc4ccccc4c3)nc(-c3cccc4c(-c5cccc6oc7ccccc7c56)cccc34)n2)cc1 ; c1ccc(-c2nc(-c3ccc4ccccc4c3)nc(-c3cc(-c4cccc5oc6ccccc6c45)c4ccccc4c3)n2)cc1]

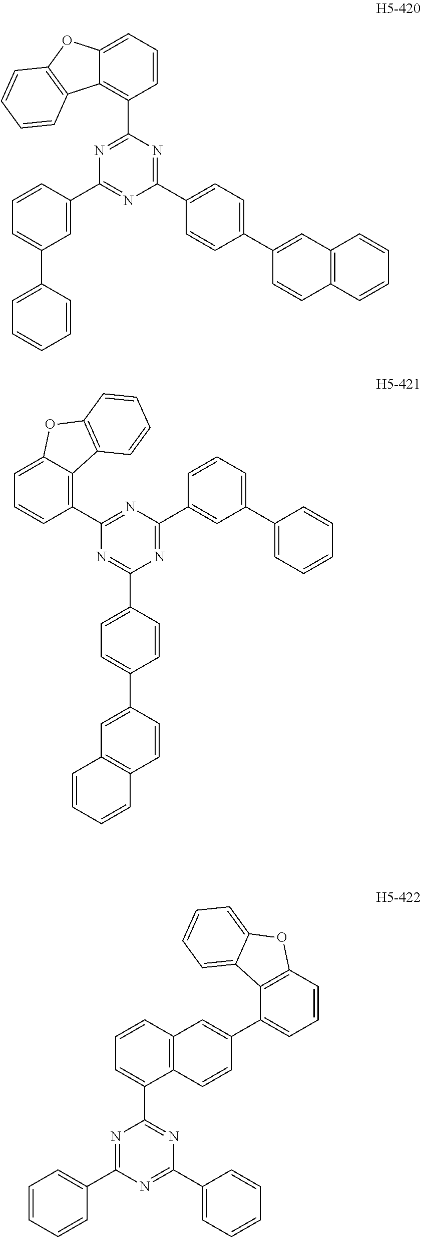 [SMILES: c1ccc(-c2cccc(-c3nc(-c4ccc(-c5ccc6ccccc6c5)cc4)nc(-c4cccc5oc6ccccc6c45)n3)c2)cc1 ; c1ccc(-c2cccc(-c3nc(-c4ccc(-c5ccc6ccccc6c5)cc4)nc(-c4cccc5oc6ccccc6c45)n3)c2)cc1 ; c1ccc(-c2nc(-c3ccccc3)nc(-c3cccc4cc(-c5cccc6oc7ccccc7c56)ccc34)n2)cc1]

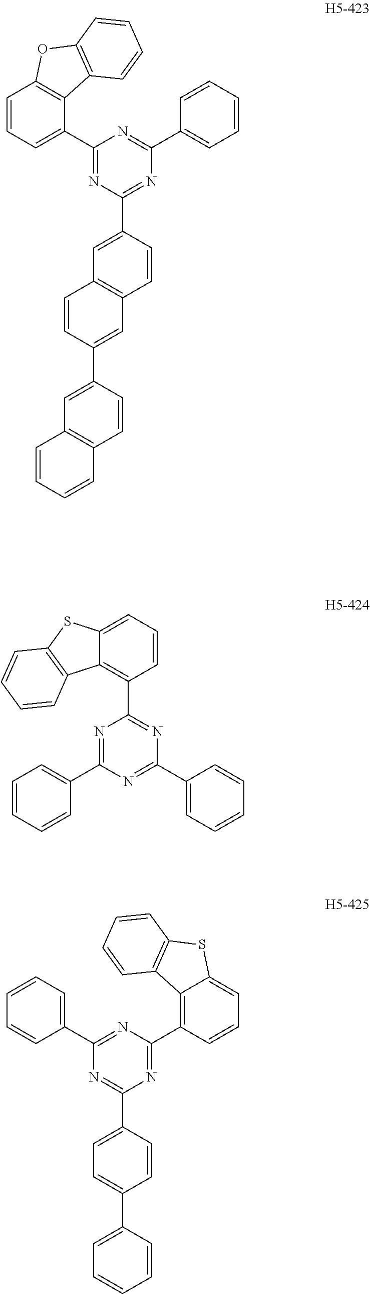 [SMILES: c1ccc(-c2nc(-c3ccc4cc(-c5ccc6ccccc6c5)ccc4c3)nc(-c3cccc4oc5ccccc5c34)n2)cc1 ; c1ccc(-c2nc(-c3ccccc3)nc(-c3cccc4sc5ccccc5c34)n2)cc1 ; c1ccc(-c2ccc(-c3nc(-c4ccccc4)nc(-c4cccc5sc6ccccc6c45)n3)cc2)cc1]

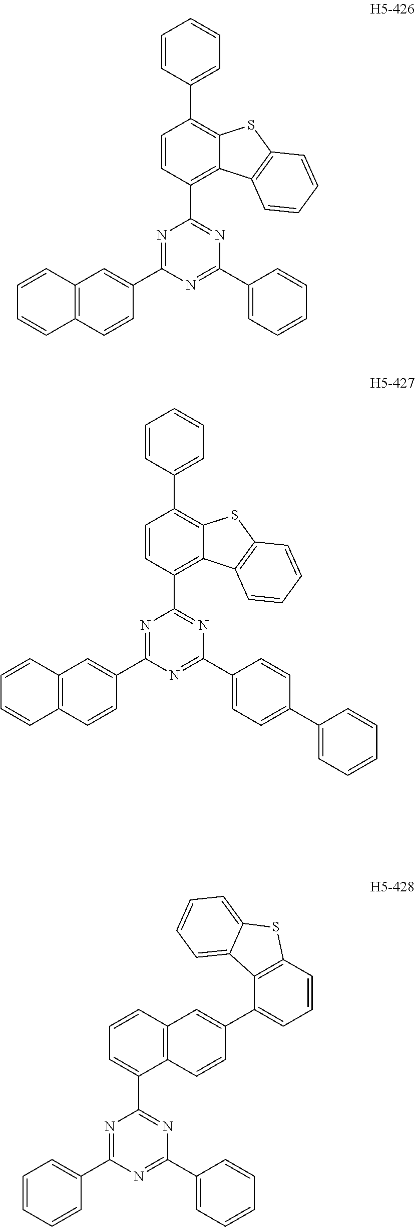 [SMILES: c1ccc(-c2nc(-c3ccc4ccccc4c3)nc(-c3ccc(-c4ccccc4)c4sc5ccccc5c34)n2)cc1 ; c1ccc(-c2ccc(-c3nc(-c4ccc5ccccc5c4)nc(-c4ccc(-c5ccccc5)c5sc6ccccc6c45)n3)cc2)cc1 ; c1ccc(-c2nc(-c3ccccc3)nc(-c3cccc4cc(-c5cccc6sc7ccccc7c56)ccc34)n2)cc1]

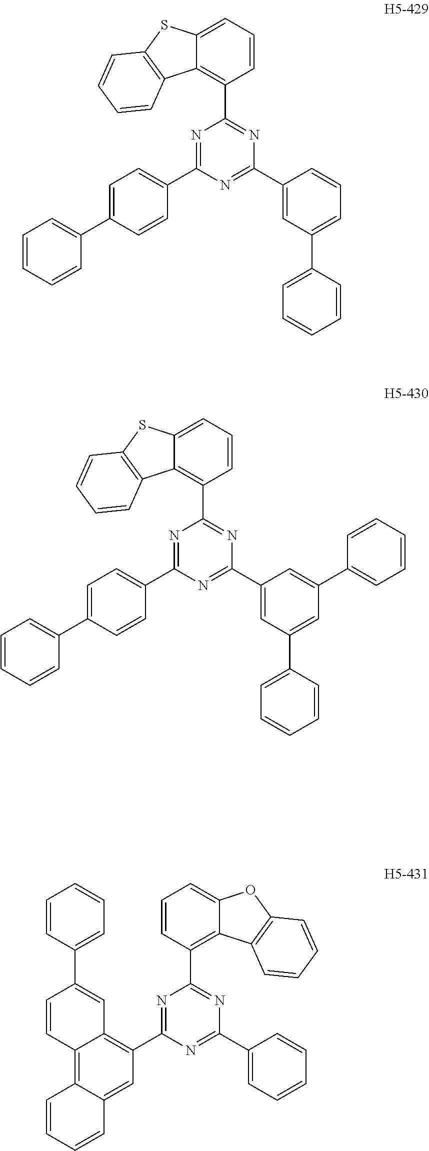 [SMILES: c1ccc(-c2ccc(-c3nc(-c4cccc(-c5ccccc5)c4)nc(-c4cccc5sc6ccccc6c45)n3)cc2)cc1 ; c1ccc(-c2ccc(-c3nc(-c4cc(-c5ccccc5)cc(-c5ccccc5)c4)nc(-c4cccc5sc6ccccc6c45)n3)cc2)cc1 ; c1ccc(-c2ccc3c(c2)c(-c2nc(-c4ccccc4)nc(-c4cccc5oc6ccccc6c45)n2)cc2ccccc23)cc1]

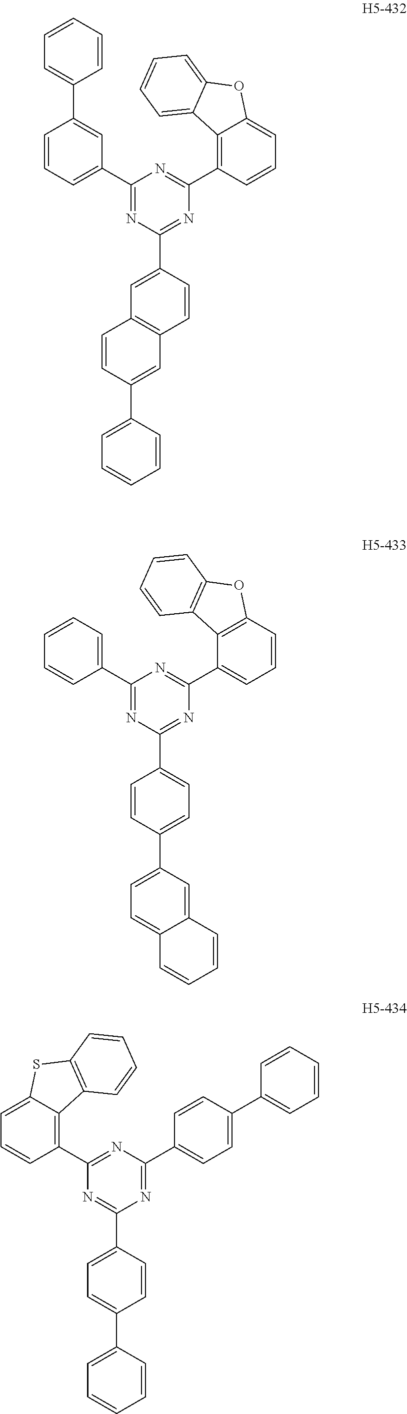 [SMILES: c1ccc(-c2cccc(-c3nc(-c4ccc5cc(-c6ccccc6)ccc5c4)nc(-c4cccc5oc6ccccc6c45)n3)c2)cc1 ; c1ccc(-c2nc(-c3ccc(-c4ccc5ccccc5c4)cc3)nc(-c3cccc4oc5ccccc5c34)n2)cc1 ; c1ccc(-c2ccc(-c3nc(-c4ccc(-c5ccccc5)cc4)nc(-c4cccc5sc6ccccc6c45)n3)cc2)cc1]

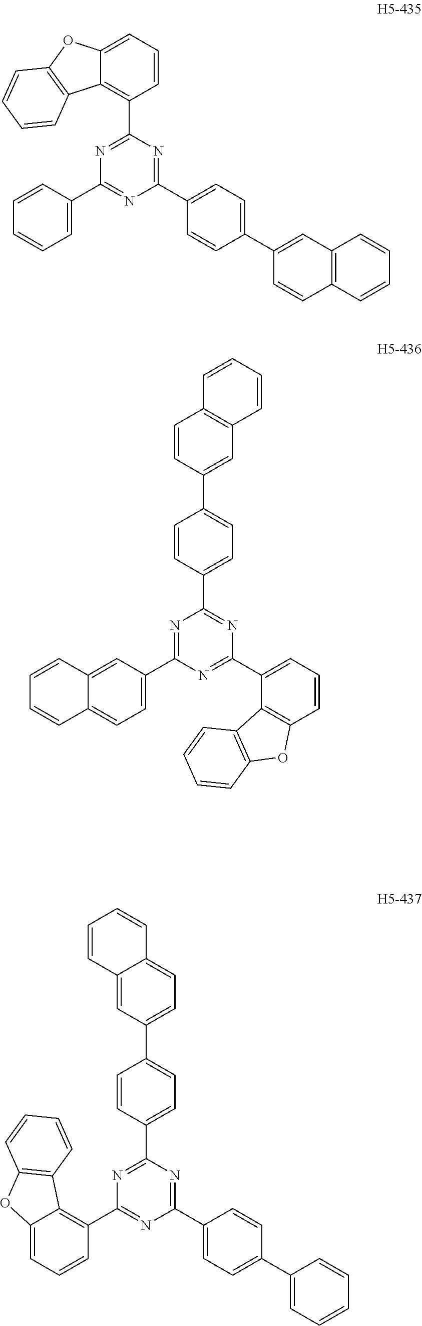 [SMILES: c1ccc(-c2nc(-c3ccc(-c4ccc5ccccc5c4)cc3)nc(-c3cccc4oc5ccccc5c34)n2)cc1 ; c1ccc2cc(-c3ccc(-c4nc(-c5ccc6ccccc6c5)nc(-c5cccc6oc7ccccc7c56)n4)cc3)ccc2c1 ; c1ccc(-c2ccc(-c3nc(-c4ccc(-c5ccc6ccccc6c5)cc4)nc(-c4cccc5oc6ccccc6c45)n3)cc2)cc1]

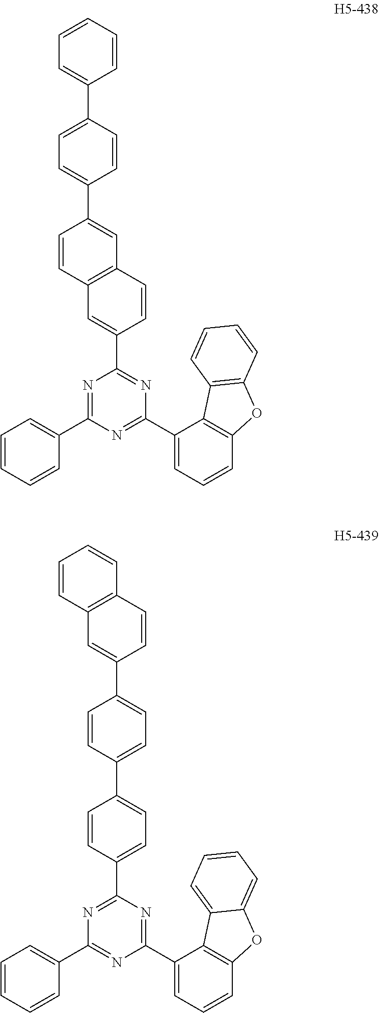 [SMILES: c1ccc(-c2ccc(-c3ccc4cc(-c5nc(-c6ccccc6)nc(-c6cccc7oc8ccccc8c67)n5)ccc4c3)cc2)cc1 ; c1ccc(-c2nc(-c3ccc(-c4ccc(-c5ccc6ccccc6c5)cc4)cc3)nc(-c3cccc4oc5ccccc5c34)n2)cc1]

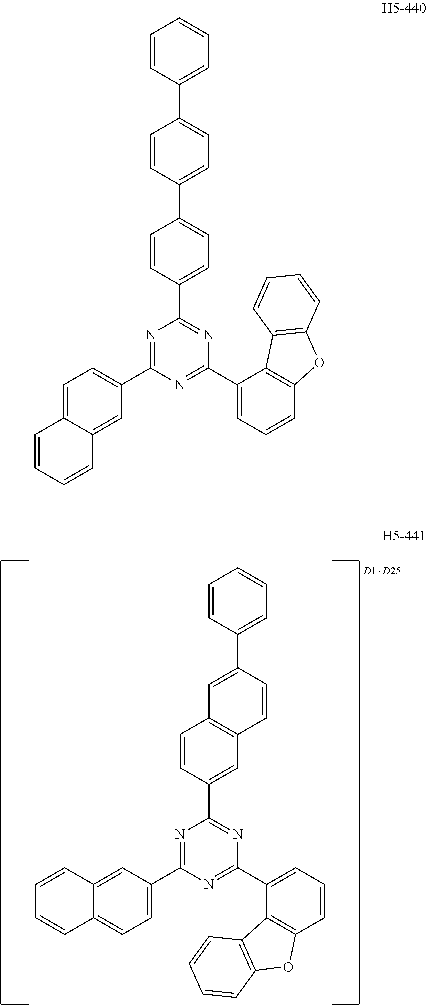

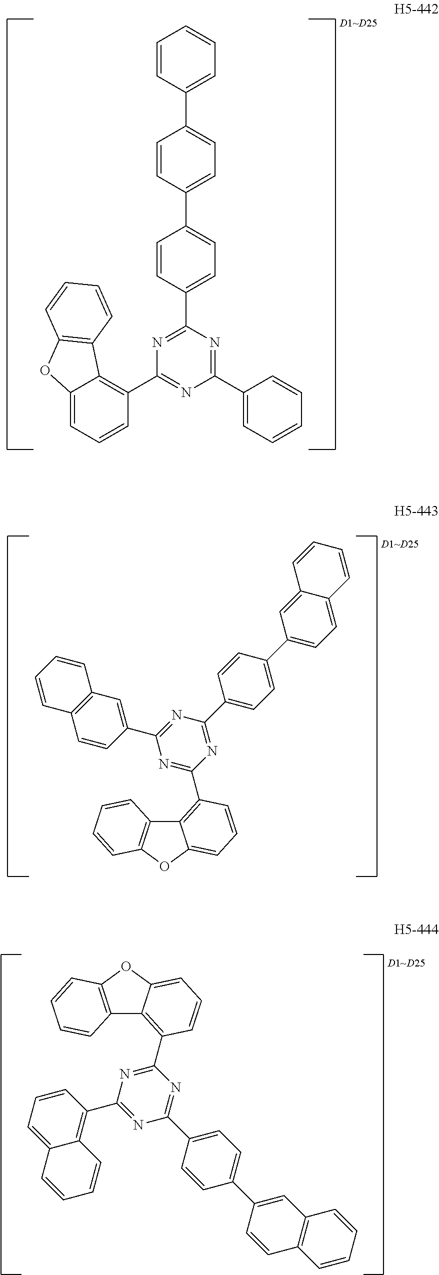

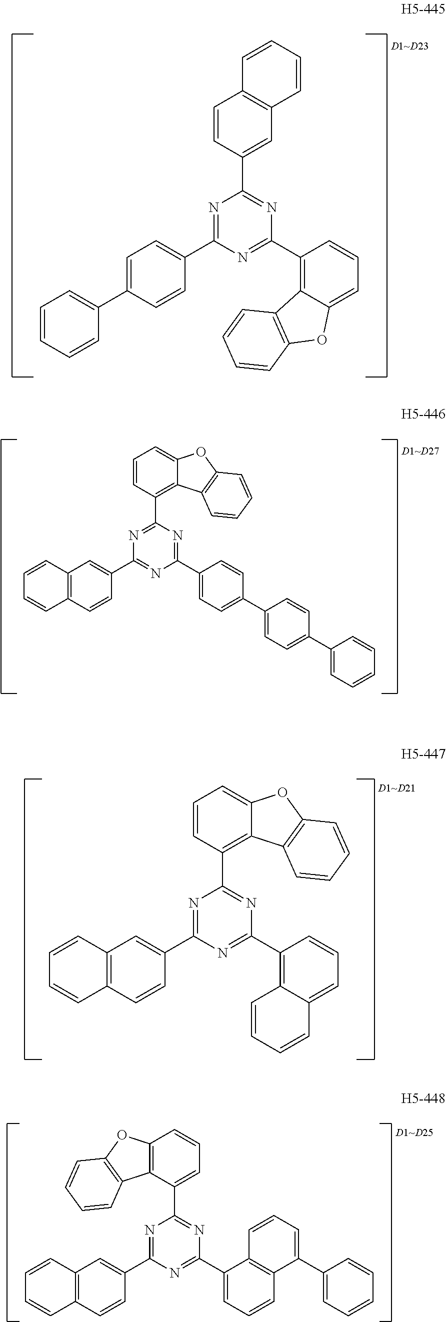

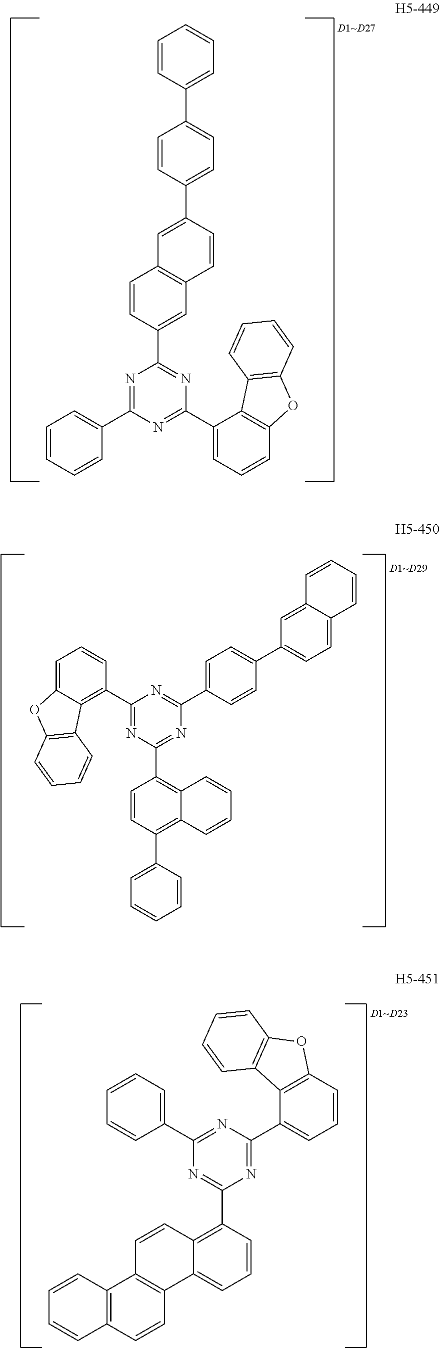

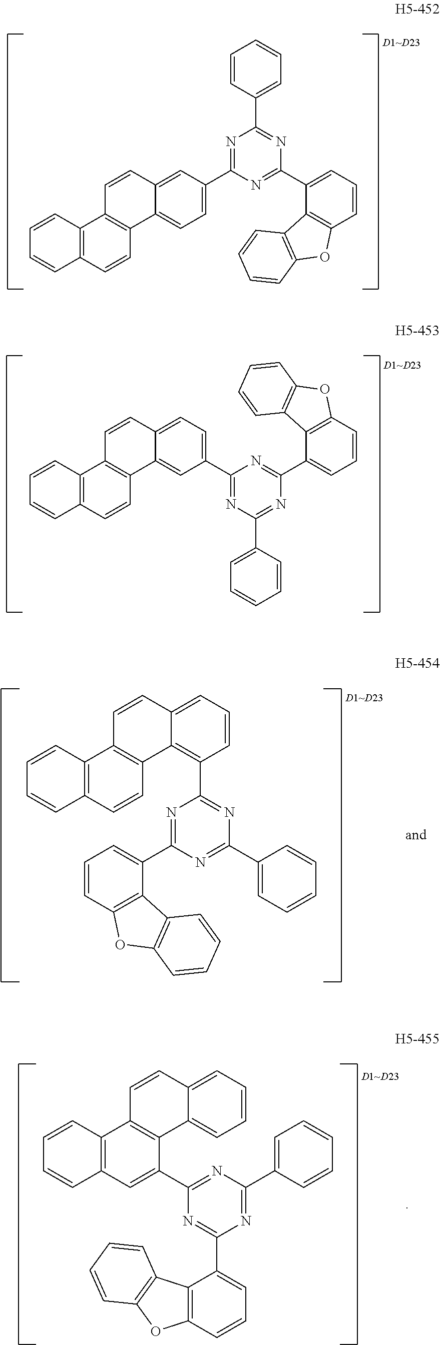

13. An organic electroluminescent device comprising: a first electrode; a second electrode; and at least one light-emitting layer(s) between the first electrode and the second electrode, wherein the at least one light-emitting layer(s) comprises the plurality of host materials according to claim 1.

14. An organic electroluminescent device comprising: a first electrode; a second electrode; and at least one light-emitting layer(s) between the first electrode and the second electrode, wherein the at least one light-emitting layer(s) comprises the plurality of host materials according to claim 8.

15. An organic electroluminescent compound represented by any one of the following Formulas 3-1-1 to 3-4-1: 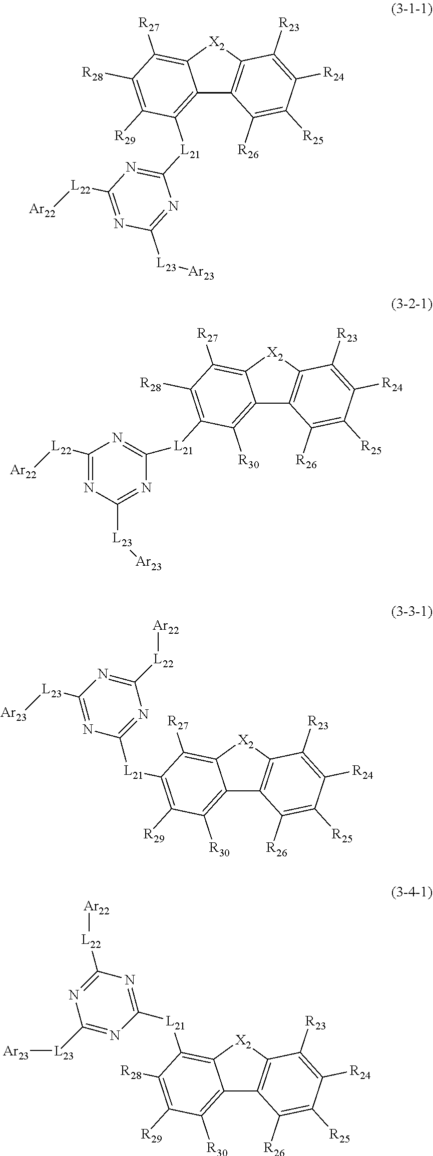
in Formulas 3-1-1 to 3-4-1,
X₂ represents O or S;
L₂₁ and L₂₂ each independently represent a single bond, a substituted or unsubstituted (C₆-C₃₀)arylene, or a substituted or unsubstituted (3- to 30-membered)heteroarylene;
L₂₃ represents phenylene unsubstituted or substituted with deuterium;
Ar₂₂ represents a substituted or unsubstituted (C₆-C₃₀)aryl, or a substituted or unsubstituted (3- to 30-membered)heteroaryl;
Ar₂₃ represents naphthyl substituted with phenyl, which may be further substituted with deuterium; and
R₂₃ to R₃₀ each independently represent hydrogen, deuterium, a halogen, a cyano, a substituted or unsubstituted (C₁-C₃₀)alkyl, a substituted or unsubstituted (C₃-C₃₀)cycloalkyl, a substituted or unsubstituted (C₆-C₃₀)aryl, a substituted or unsubstituted (3- to 30-membered)heteroaryl, a substituted or unsubstituted (C₁-C₃₀)alkoxy, a substituted or unsubstituted tri(C₁-C₃₀)alkylsilyl, a substituted or unsubstituted di(C₁-C₃₀)alkyl(C₆-C₃₀)arylsilyl, a substituted or unsubstituted (C₁-C₃₀)alkyldi(C₆-C₃₀)arylsilyl, a substituted or unsubstituted tri(C₆-C₃₀)arylsilyl, a substituted or unsubstituted mono- or di(C₁-C₃₀)alkylamino, a substituted or unsubstituted mono- or di(C₂-C₃₀)alkenylamino, a substituted or unsubstituted (C₁-C₃₀)alkyl(C₂-C₃₀)alkenylamino, a substituted or unsubstituted mono- or di(C₆-C₃₀)arylamino, a substituted or unsubstituted (C₁-C₃₀)alkyl(C₆-C₃₀)arylamino, a substituted or unsubstituted mono- or di(3- to 30-membered)heteroarylamino, a substituted or unsubstituted (C₁-C₃₀)alkyl(3- to 30-membered)heteroarylamino, a substituted or unsubstituted (C₂-C₃₀)alkenyl(C₆-C₃₀)arylamino, a substituted or unsubstituted (C₂-C₃₀)alkenyl(3- to 30-membered)heteroarylamino, a substituted or unsubstituted (C₆-C₃₀)aryl(3- to 30-membered)heteroarylamino, or a substituted or unsubstituted fused ring of (C₃-C₃₀) aliphatic ring and (C₆-C₃₀) aromatic ring;
provided that in each of Formulas 3-1-1 to 3-4-1, at least one of R₂₃ to R₃₀ is a substituted or unsubstituted (C₆-C₃₀)aryl, or a substituted or unsubstituted (3- to 30-membered)heteroaryl.

16. The organic electroluminescent compound according to claim 15, wherein Ar₂₃ is represented by the following Formula A: 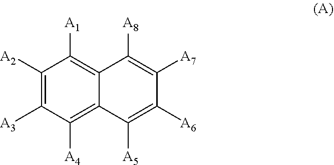
in Formula A,
any one of A₁ to A₈ is linked to L₂₃; A₁ to A₈ which are not linked to L₂₃, each independently represent hydrogen, deuterium, or a substituted or unsubstituted phenyl;
provided that at least one of A₁ to A₈ is a substituted or unsubstituted phenyl.

17. The organic electroluminescent compound according to claim 15, wherein the compound represented by any one of Formulas 3-1-1 to 3-4-1 is selected from the following compounds:

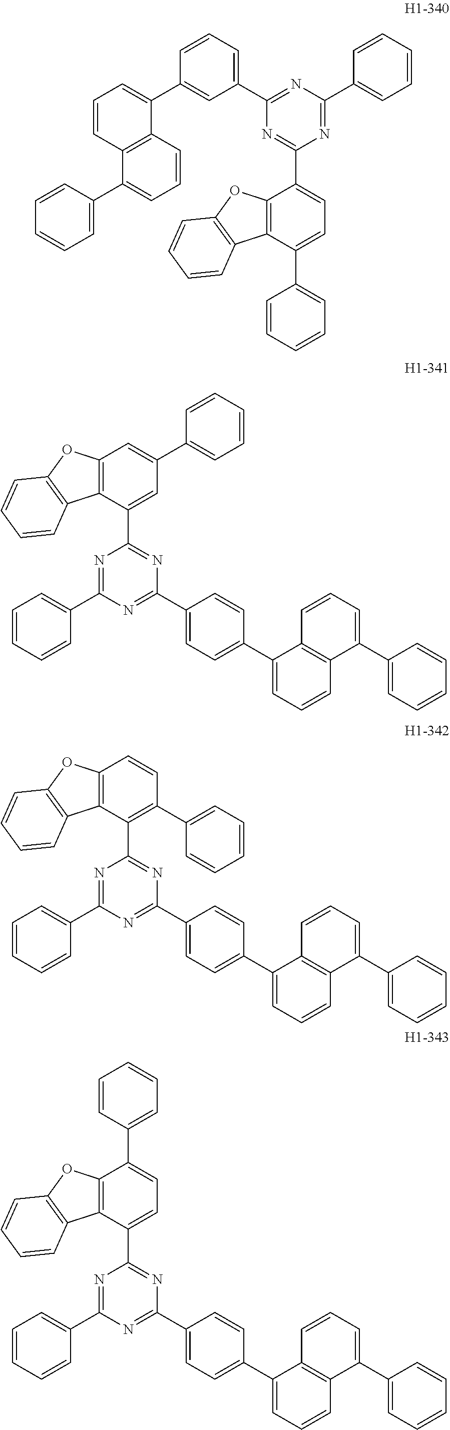

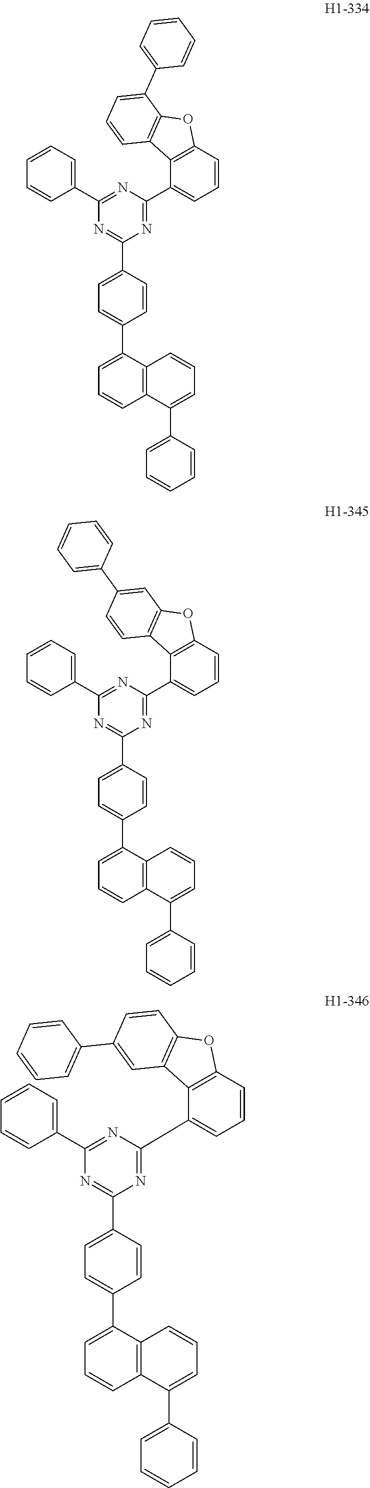

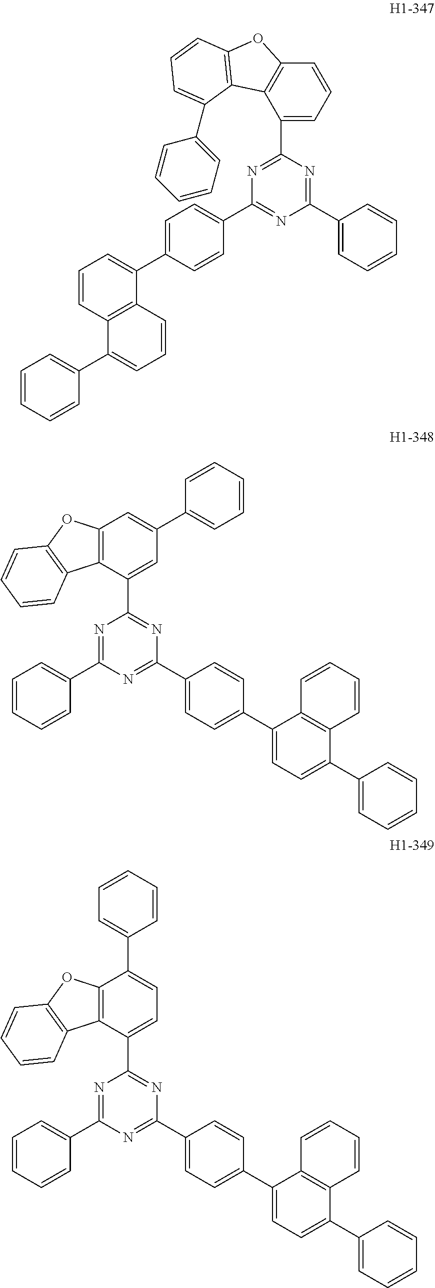

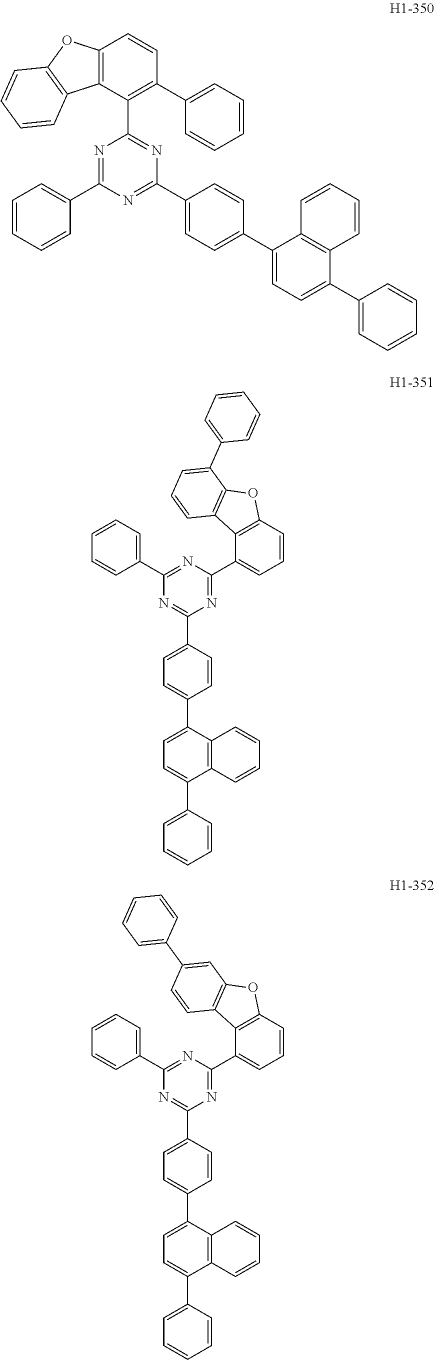 [SMILES: c1ccc(-c2nc(-c3ccc(-c4ccc(-c5ccccc5)c5ccccc45)cc3)nc(-c3c(-c4ccccc4)ccc4oc5ccccc5c34)n2)cc1 ; c1ccc(-c2nc(-c3ccc(-c4ccc(-c5ccccc5)c5ccccc45)cc3)nc(-c3cccc4oc5c(-c6ccccc6)cccc5c34)n2)cc1 ; c1ccc(-c2ccc3c(c2)oc2cccc(-c4nc(-c5ccccc5)nc(-c5ccc(-c6ccc(-c7ccccc7)c7ccccc67)cc5)n4)c23)cc1]

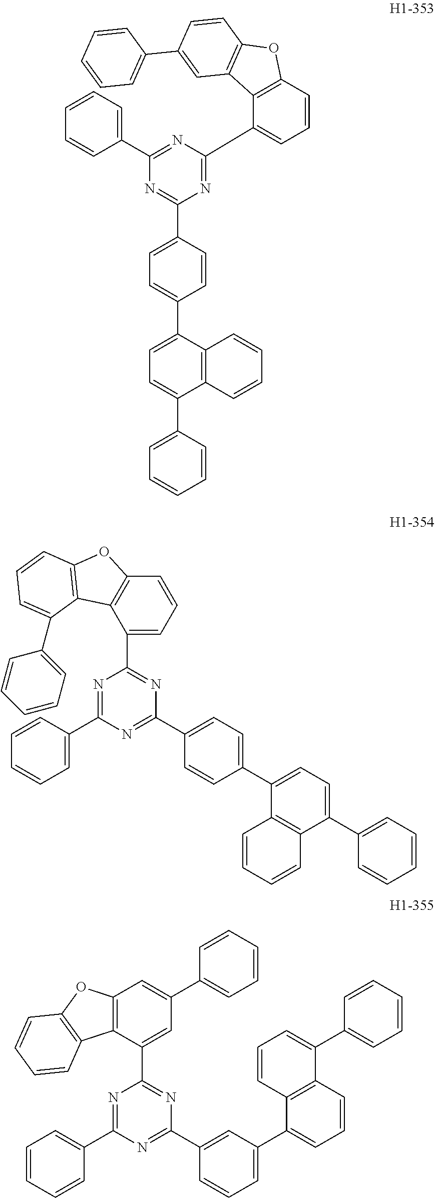

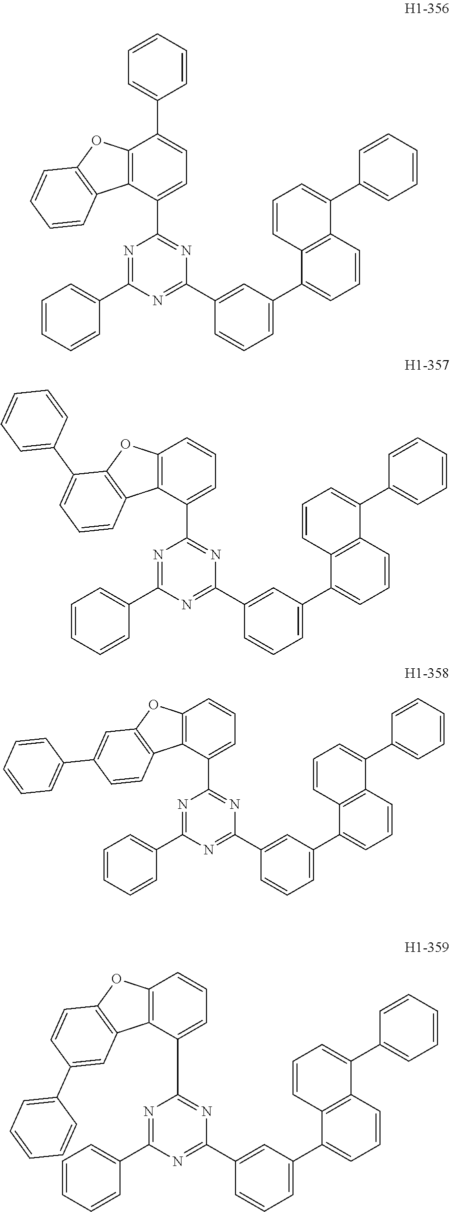

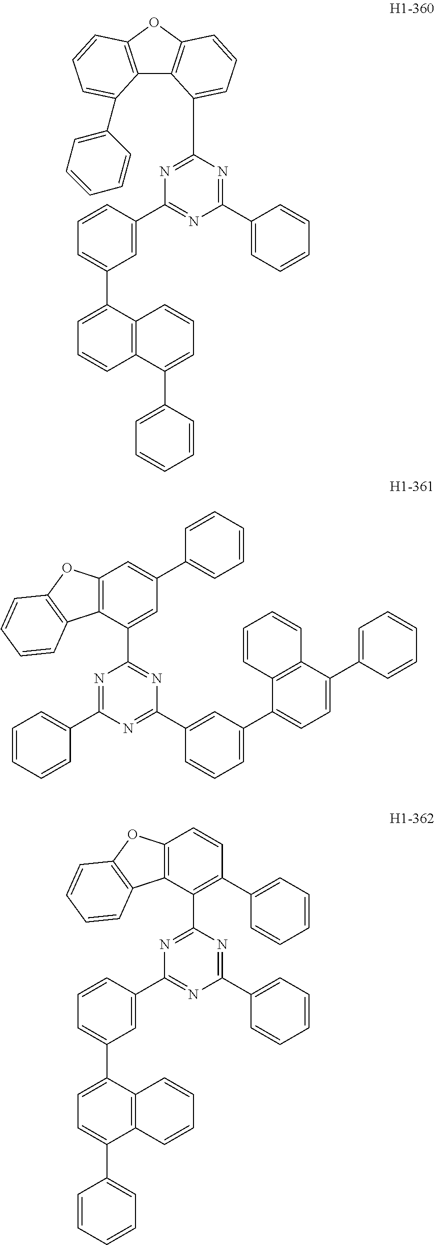 [SMILES: c1ccc(-c2nc(-c3cccc(-c4cccc5c(-c6ccccc6)cccc45)c3)nc(-c3cccc4oc5cccc(-c6ccccc6)c5c34)n2)cc1 ; c1ccc(-c2cc(-c3nc(-c4ccccc4)nc(-c4cccc(-c5ccc(-c6ccccc6)c6ccccc56)c4)n3)c3c(c2)oc2ccccc23)cc1 ; c1ccc(-c2nc(-c3cccc(-c4ccc(-c5ccccc5)c5ccccc45)c3)nc(-c3c(-c4ccccc4)ccc4oc5ccccc5c34)n2)cc1]

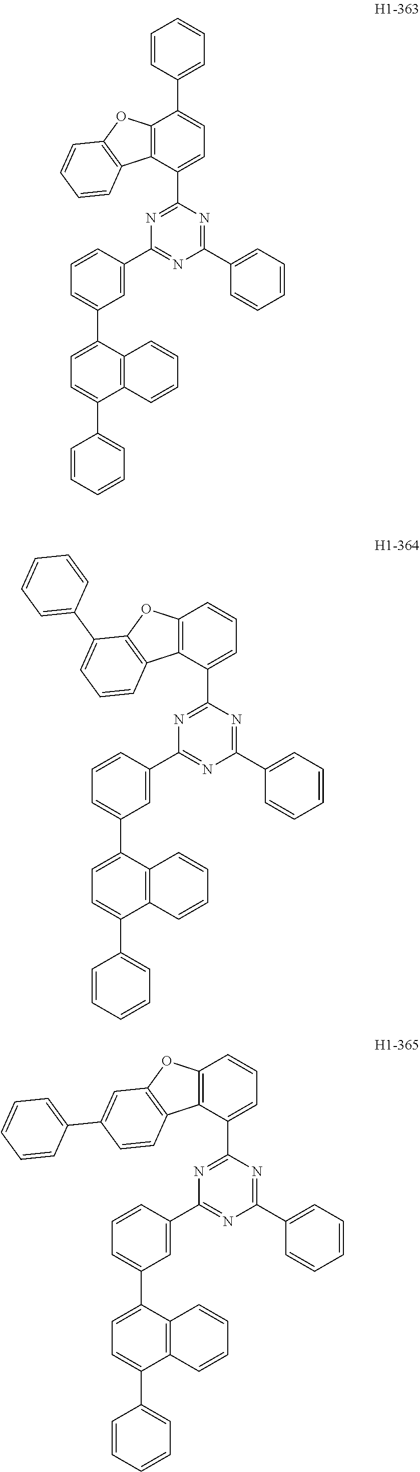 [SMILES: c1ccc(-c2nc(-c3cccc(-c4ccc(-c5ccccc5)c5ccccc45)c3)nc(-c3ccc(-c4ccccc4)c4oc5ccccc5c34)n2)cc1 ; c1ccc(-c2nc(-c3cccc(-c4ccc(-c5ccccc5)c5ccccc45)c3)nc(-c3cccc4oc5c(-c6ccccc6)cccc5c34)n2)cc1 ; c1ccc(-c2ccc3c(c2)oc2cccc(-c4nc(-c5ccccc5)nc(-c5cccc(-c6ccc(-c7ccccc7)c7ccccc67)c5)n4)c23)cc1]

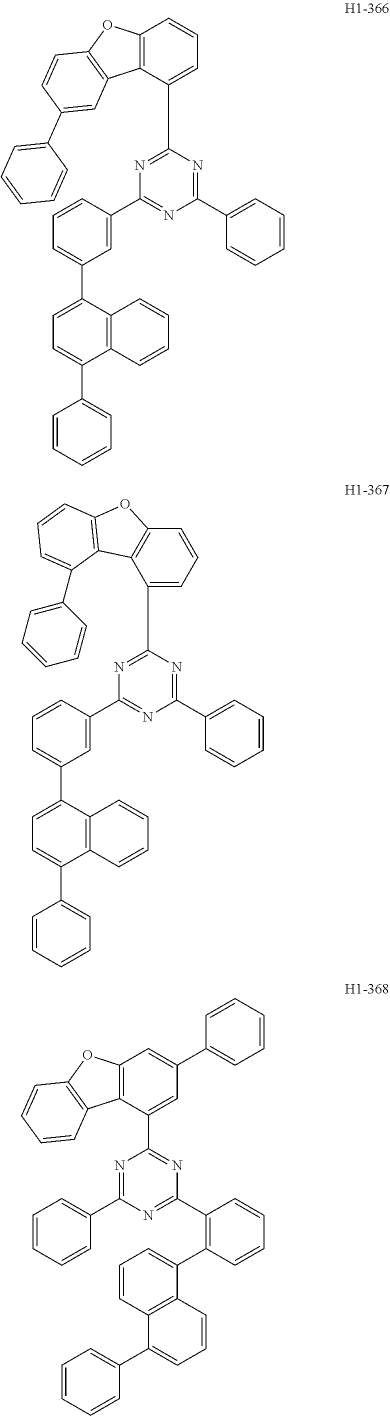

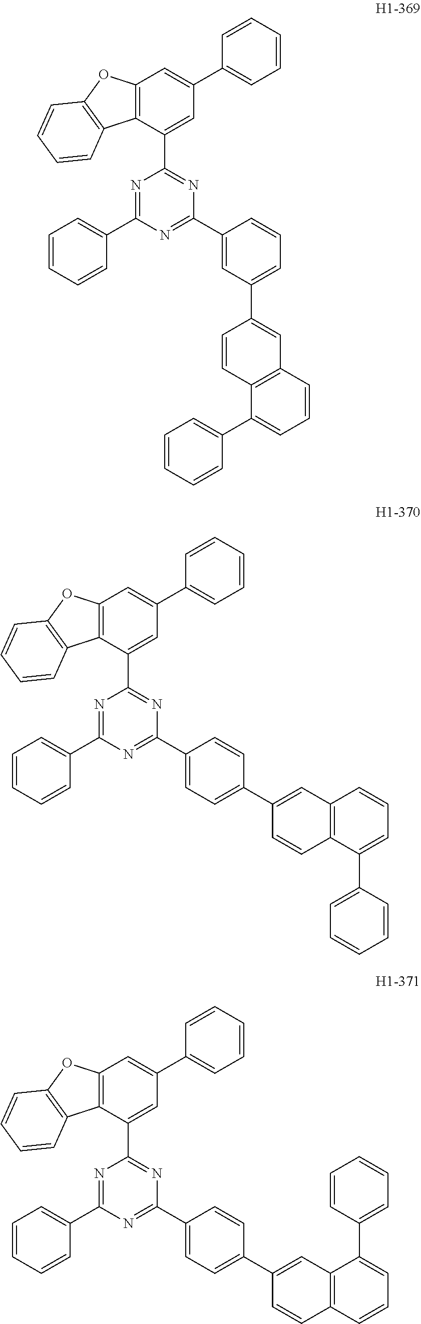 [SMILES: c1ccc(-c2cc(-c3nc(-c4ccccc4)nc(-c4cccc(-c5ccc6c(-c7ccccc7)cccc6c5)c4)n3)c3c(c2)oc2ccccc23)cc1 ; c1ccc(-c2cc(-c3nc(-c4ccccc4)nc(-c4ccc(-c5ccc6c(-c7ccccc7)cccc6c5)cc4)n3)c3c(c2)oc2ccccc23)cc1 ; c1ccc(-c2cc(-c3nc(-c4ccccc4)nc(-c4ccc(-c5ccc6cccc(-c7ccccc7)c6c5)cc4)n3)c3c(c2)oc2ccccc23)cc1]

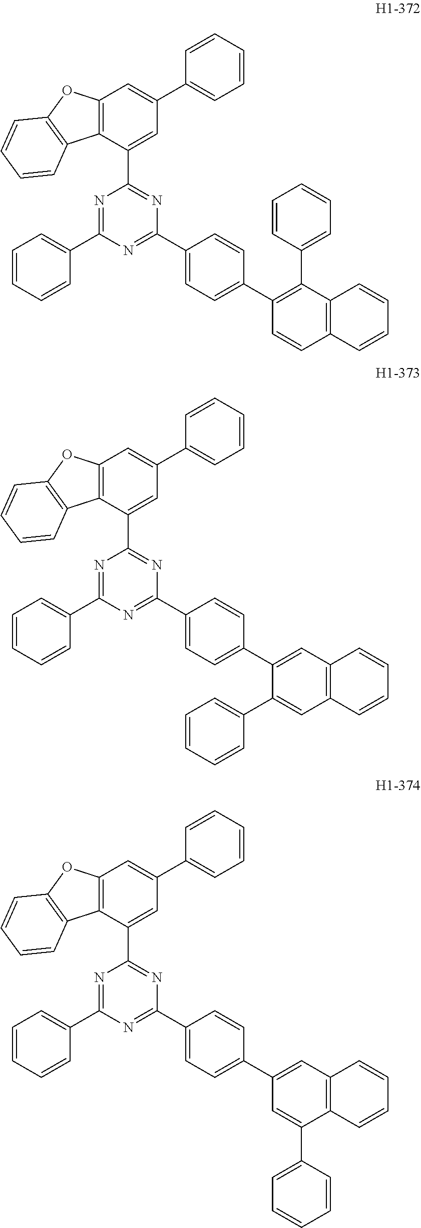

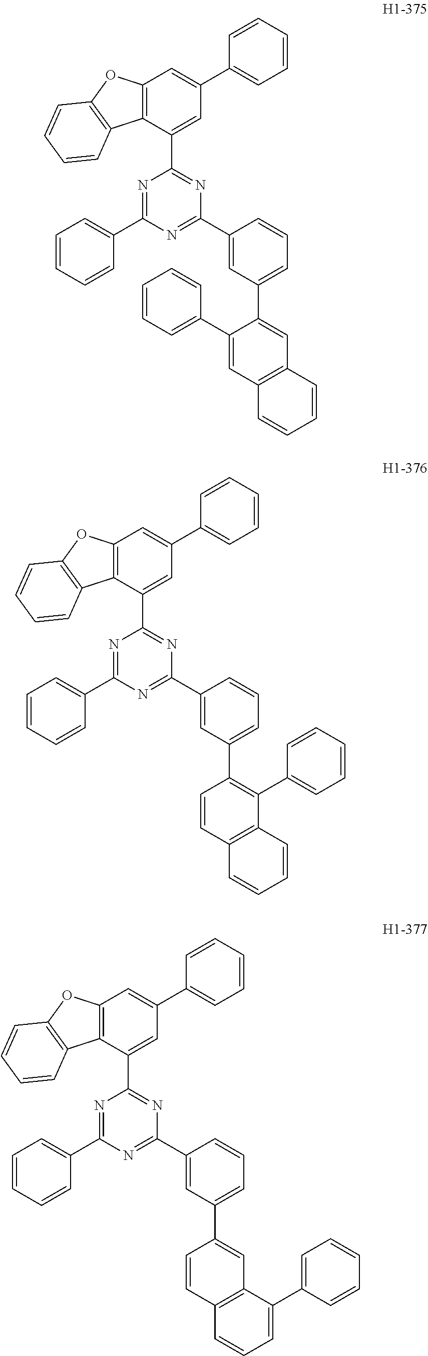 [SMILES: c1ccc(-c2cc(-c3nc(-c4ccccc4)nc(-c4cccc(-c5cc6ccccc6cc5-c5ccccc5)c4)n3)c3c(c2)oc2ccccc23)cc1 ; c1ccc(-c2cc(-c3nc(-c4ccccc4)nc(-c4cccc(-c5ccc6ccccc6c5-c5ccccc5)c4)n3)c3c(c2)oc2ccccc23)cc1 ; c1ccc(-c2cc(-c3nc(-c4ccccc4)nc(-c4cccc(-c5ccc6cccc(-c7ccccc7)c6c5)c4)n3)c3c(c2)oc2ccccc23)cc1]

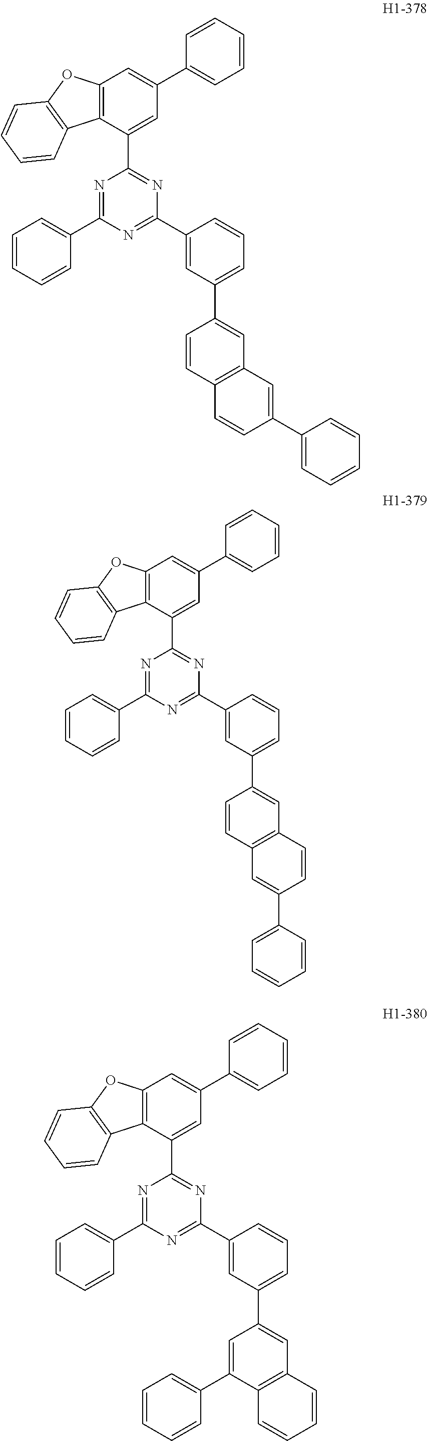

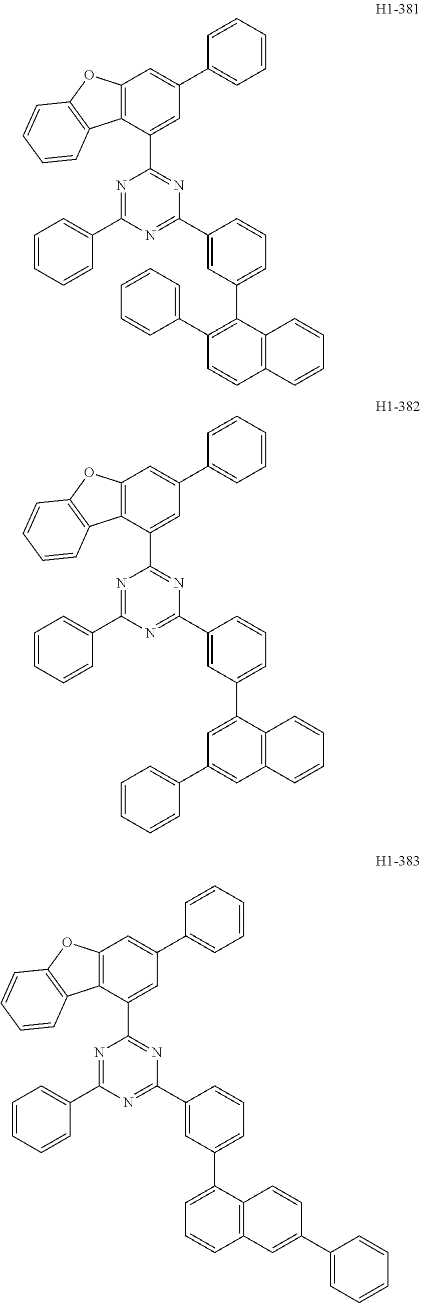

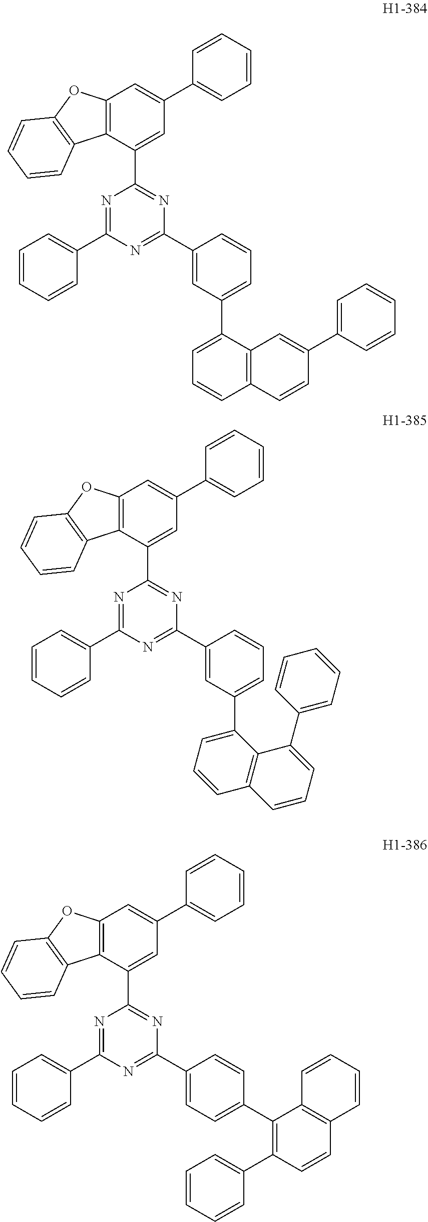

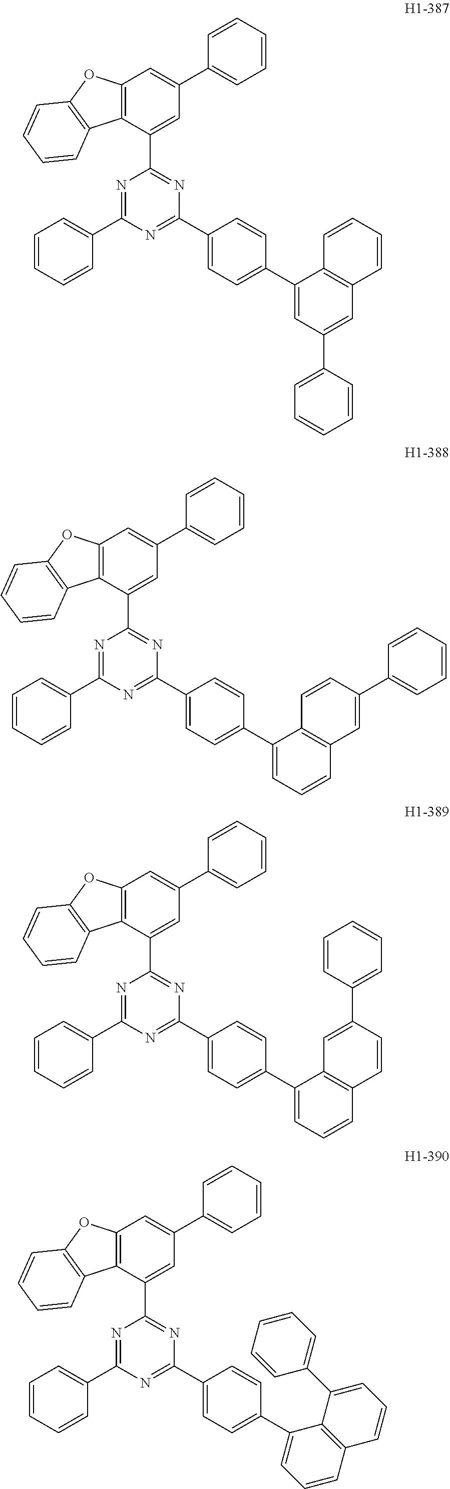 [SMILES: c1ccc(-c2cc(-c3ccc(-c4nc(-c5ccccc5)nc(-c5cc(-c6ccccc6)cc6oc7ccccc7c56)n4)cc3)c3ccccc3c2)cc1 ; c1ccc(-c2ccc3c(-c4ccc(-c5nc(-c6ccccc6)nc(-c6cc(-c7ccccc7)cc7oc8ccccc8c67)n5)cc4)cccc3c2)cc1 ; c1ccc(-c2cc(-c3nc(-c4ccccc4)nc(-c4ccc(-c5cccc6ccc(-c7ccccc7)cc56)cc4)n3)c3c(c2)oc2ccccc23)cc1 ; c1ccc(-c2cc(-c3nc(-c4ccccc4)nc(-c4ccc(-c5cccc6cccc(-c7ccccc7)c56)cc4)n3)c3c(c2)oc2ccccc23)cc1]

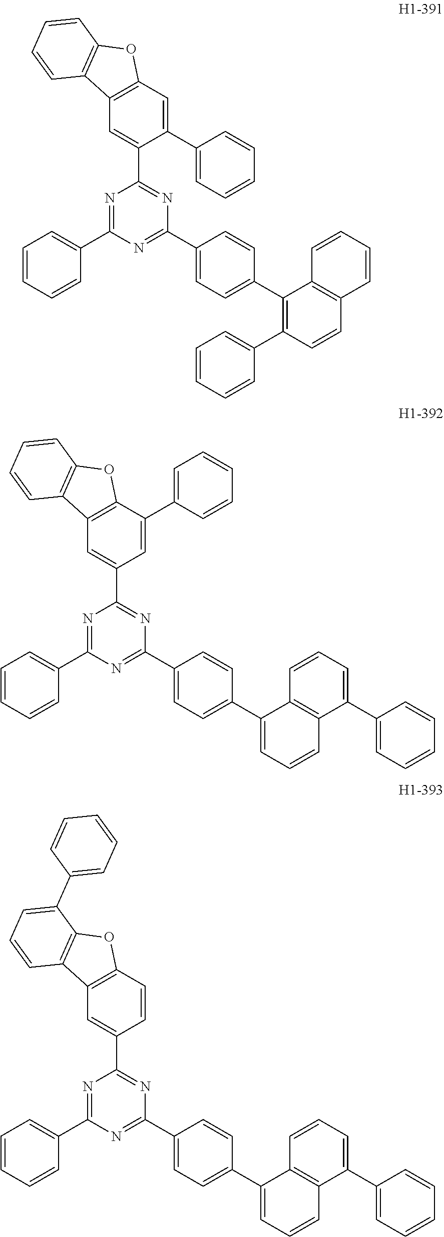 [SMILES: c1ccc(-c2nc(-c3ccc(-c4c(-c5ccccc5)ccc5ccccc45)cc3)nc(-c3cc4c(cc3-c3ccccc3)oc3ccccc34)n2)cc1 ; c1ccc(-c2nc(-c3ccc(-c4cccc5c(-c6ccccc6)cccc45)cc3)nc(-c3cc(-c4ccccc4)c4oc5ccccc5c4c3)n2)cc1 ; c1ccc(-c2nc(-c3ccc(-c4cccc5c(-c6ccccc6)cccc45)cc3)nc(-c3ccc4oc5c(-c6ccccc6)cccc5c4c3)n2)cc1]

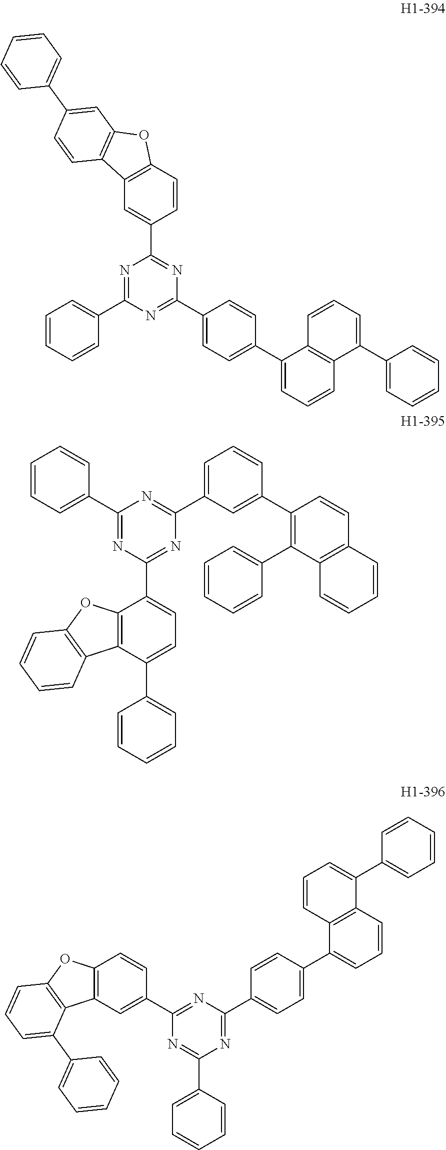 [SMILES: c1ccc(-c2ccc3c(c2)oc2ccc(-c4nc(-c5ccccc5)nc(-c5ccc(-c6cccc7c(-c8ccccc8)cccc67)cc5)n4)cc23)cc1 ; c1ccc(-c2nc(-c3cccc(-c4ccc5ccccc5c4-c4ccccc4)c3)nc(-c3ccc(-c4ccccc4)c4c3oc3ccccc34)n2)cc1 ; c1ccc(-c2nc(-c3ccc(-c4cccc5c(-c6ccccc6)cccc45)cc3)nc(-c3ccc4oc5cccc(-c6ccccc6)c5c4c3)n2)cc1]

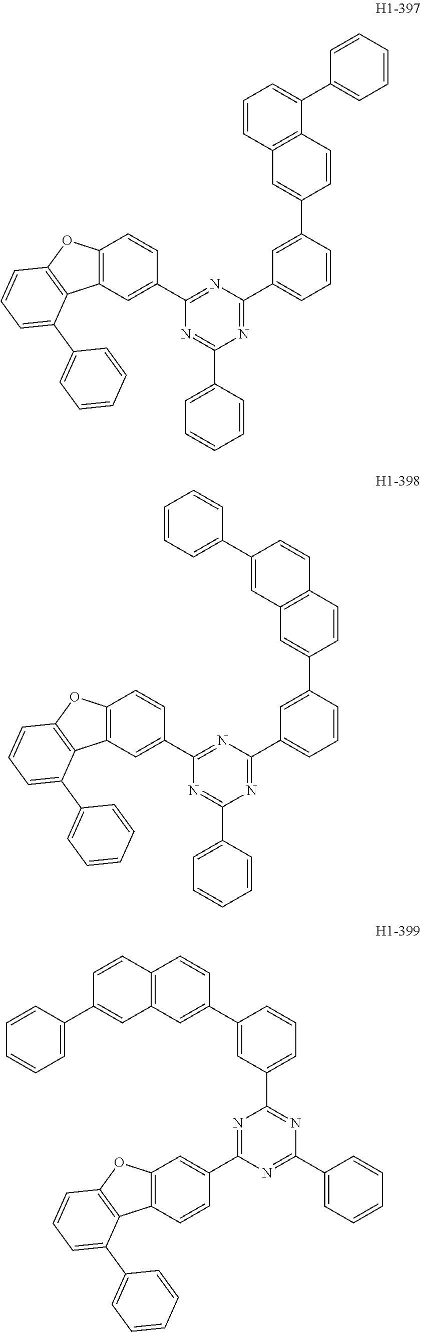

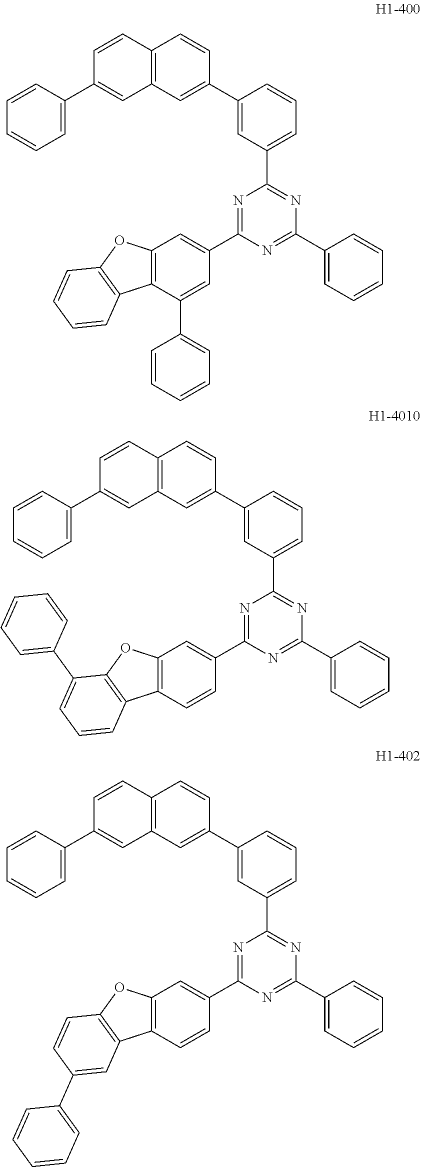

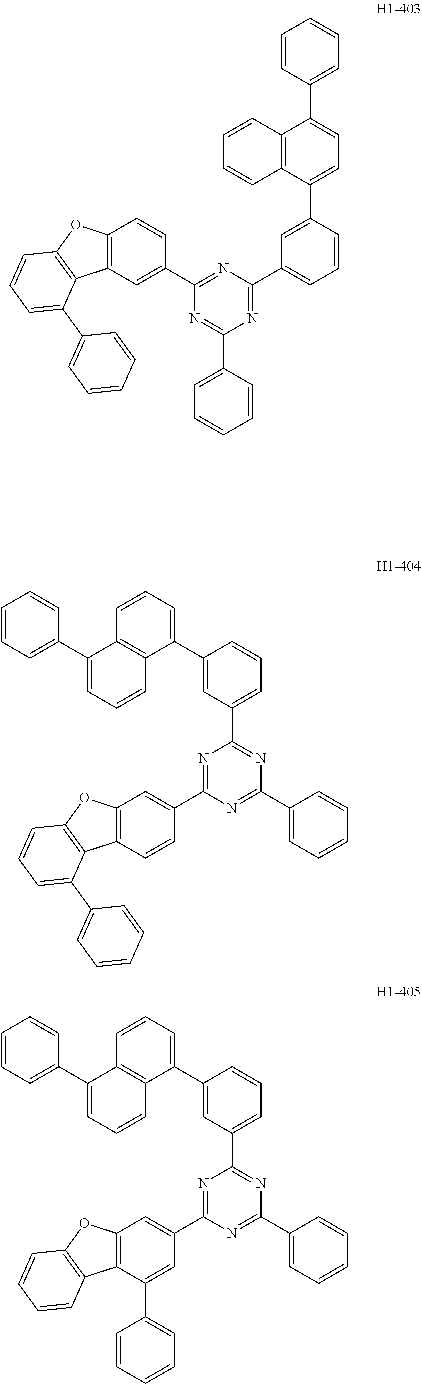 [SMILES: c1ccc(-c2nc(-c3cccc(-c4ccc(-c5ccccc5)c5ccccc45)c3)nc(-c3ccc4oc5cccc(-c6ccccc6)c5c4c3)n2)cc1 ; c1ccc(-c2nc(-c3cccc(-c4cccc5c(-c6ccccc6)cccc45)c3)nc(-c3ccc4c(c3)oc3cccc(-c5ccccc5)c34)n2)cc1 ; c1ccc(-c2nc(-c3cccc(-c4cccc5c(-c6ccccc6)cccc45)c3)nc(-c3cc(-c4ccccc4)c4c(c3)oc3ccccc34)n2)cc1]

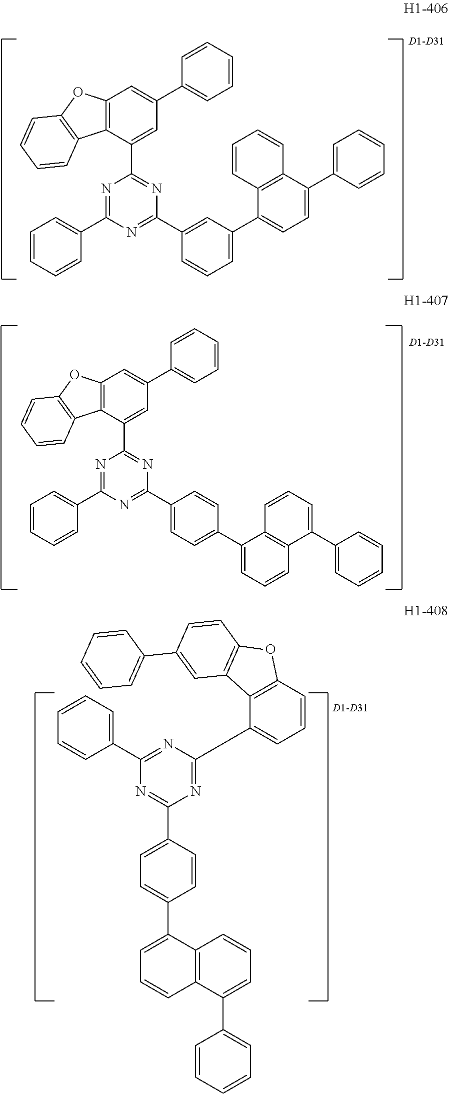

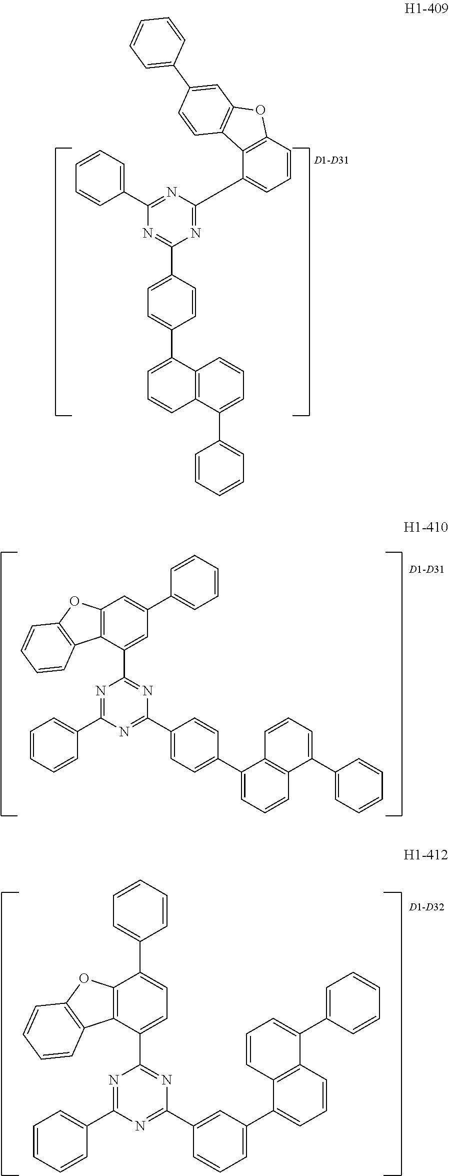

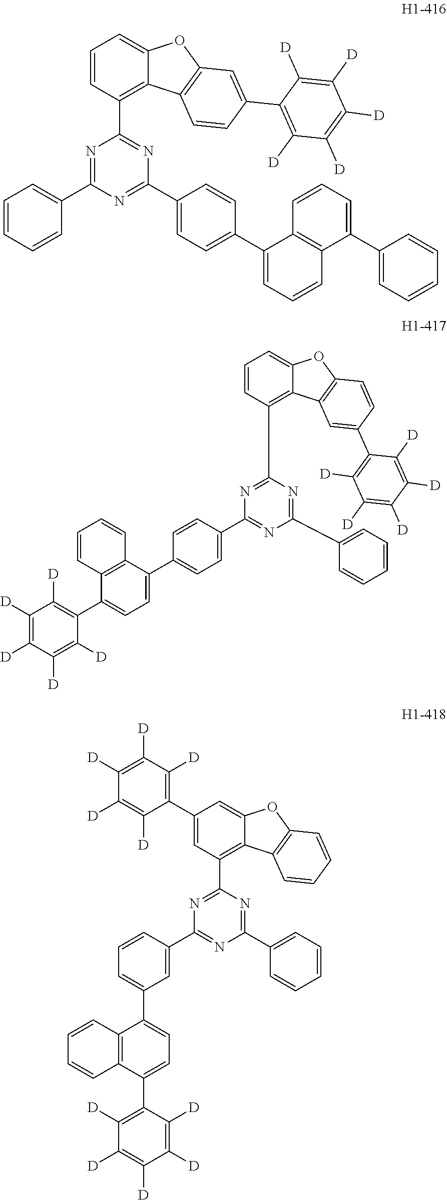 [SMILES: [2H]c1c([2H])c([2H])c(-c2ccc3c(c2)oc2cccc(-c4nc(-c5ccccc5)nc(-c5ccc(-c6cccc7c(-c8ccccc8)cccc67)cc5)n4)c23)c([2H])c1[2H] ; [2H]c1c([2H])c([2H])c(-c2ccc3oc4cccc(-c5nc(-c6ccccc6)nc(-c6ccc(-c7ccc(-c8c([2H])c([2H])c([2H])c([2H])c8[2H])c8ccccc78)cc6)n5)c4c3c2)c([2H])c1[2H] ; [2H]c1c([2H])c([2H])c(-c2cc(-c3nc(-c4ccccc4)nc(-c4cccc(-c5ccc(-c6c([2H])c([2H])c([2H])c([2H])c6[2H])c6ccccc56)c4)n3)c3c(c2)oc2ccccc23)c([2H])c1[2H]]

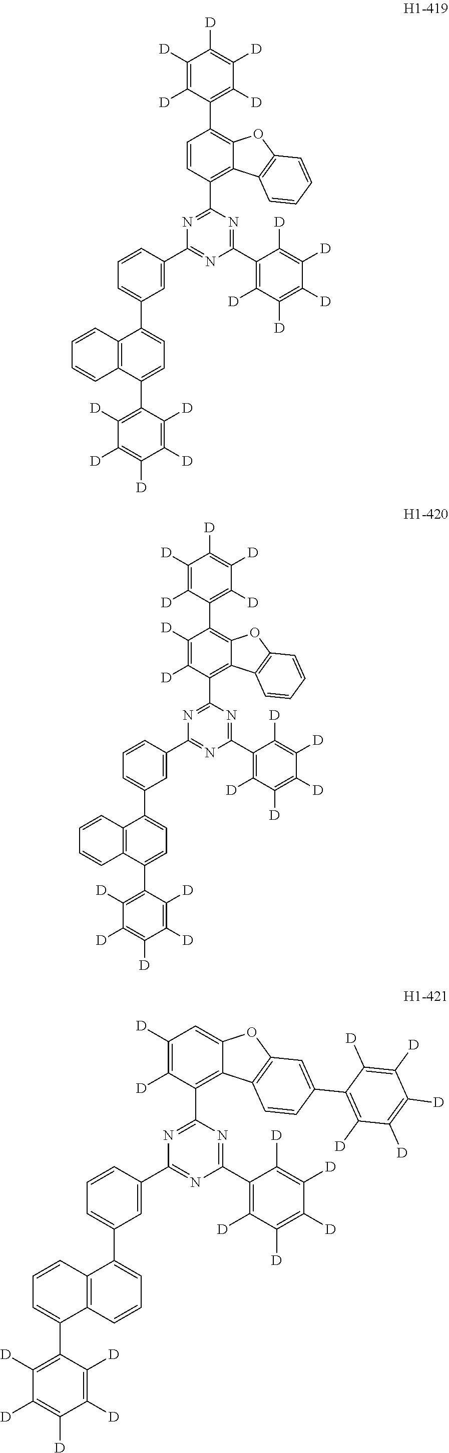

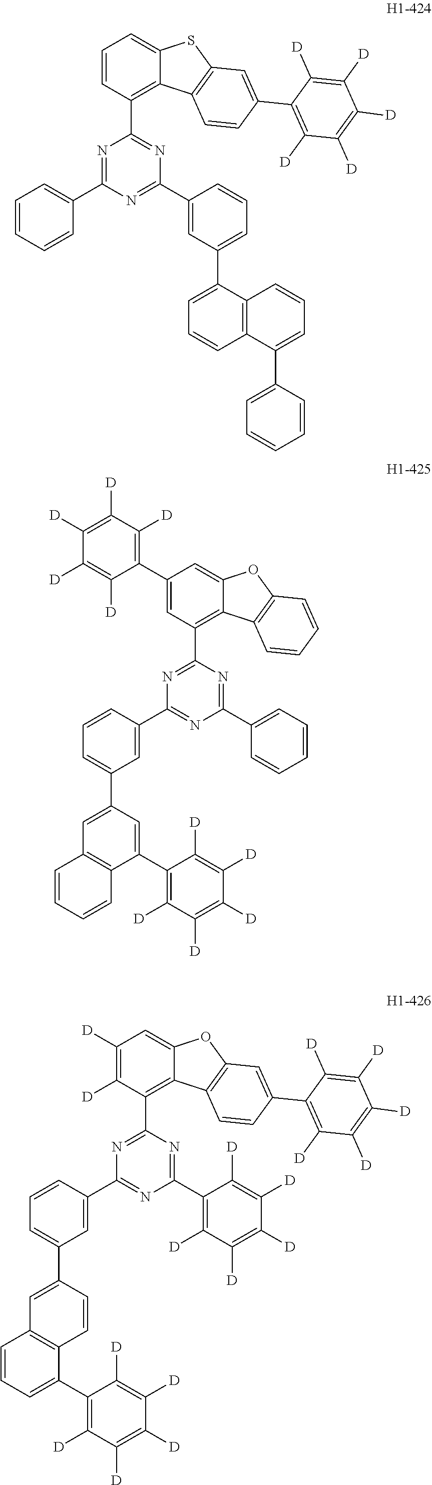 [SMILES: [2H]c1c([2H])c([2H])c(-c2ccc3c(c2)sc2cccc(-c4nc(-c5ccccc5)nc(-c5cccc(-c6cccc7c(-c8ccccc8)cccc67)c5)n4)c23)c([2H])c1[2H] ; [2H]c1c([2H])c([2H])c(-c2cc(-c3nc(-c4ccccc4)nc(-c4cccc(-c5cc(-c6c([2H])c([2H])c([2H])c([2H])c6[2H])c6ccccc6c5)c4)n3)c3c(c2)oc2ccccc23)c([2H])c1[2H] ; [2H]c1cc2oc3cc(-c4c([2H])c([2H])c([2H])c([2H])c4[2H])ccc3c2c(-c2nc(-c3cccc(-c4ccc5c(-c6c([2H])c([2H])c([2H])c([2H])c6[2H])cccc5c4)c3)nc(-c3c([2H])c([2H])c([2H])c([2H])c3[2H])n2)c1[2H]]

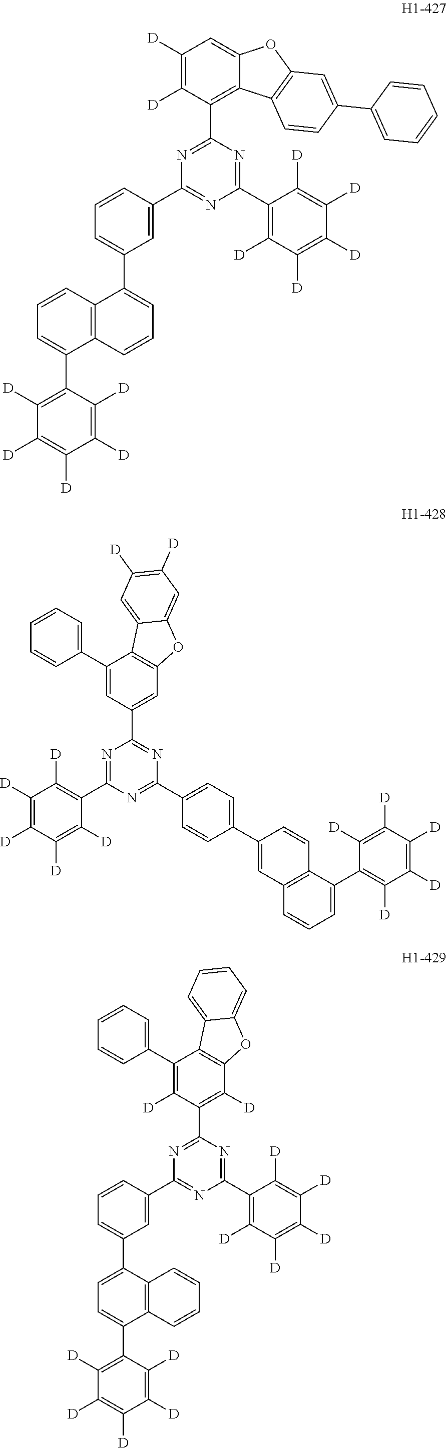 [SMILES: [2H]c1cc2oc3cc(-c4ccccc4)ccc3c2c(-c2nc(-c3cccc(-c4cccc5c(-c6c([2H])c([2H])c([2H])c([2H])c6[2H])cccc45)c3)nc(-c3c([2H])c([2H])c([2H])c([2H])c3[2H])n2)c1[2H] ; [2H]c1cc2oc3cc(-c4nc(-c5ccc(-c6ccc7c(-c8c([2H])c([2H])c([2H])c([2H])c8[2H])cccc7c6)cc5)nc(-c5c([2H])c([2H])c([2H])c([2H])c5[2H])n4)cc(-c4ccccc4)c3c2cc1[2H] ; [2H]c1c([2H])c([2H])c(-c2nc(-c3cccc(-c4ccc(-c5c([2H])c([2H])c([2H])c([2H])c5[2H])c5ccccc45)c3)nc(-c3c([2H])c(-c4ccccc4)c4c(oc5ccccc54)c3[2H])n2)c([2H])c1[2H]]

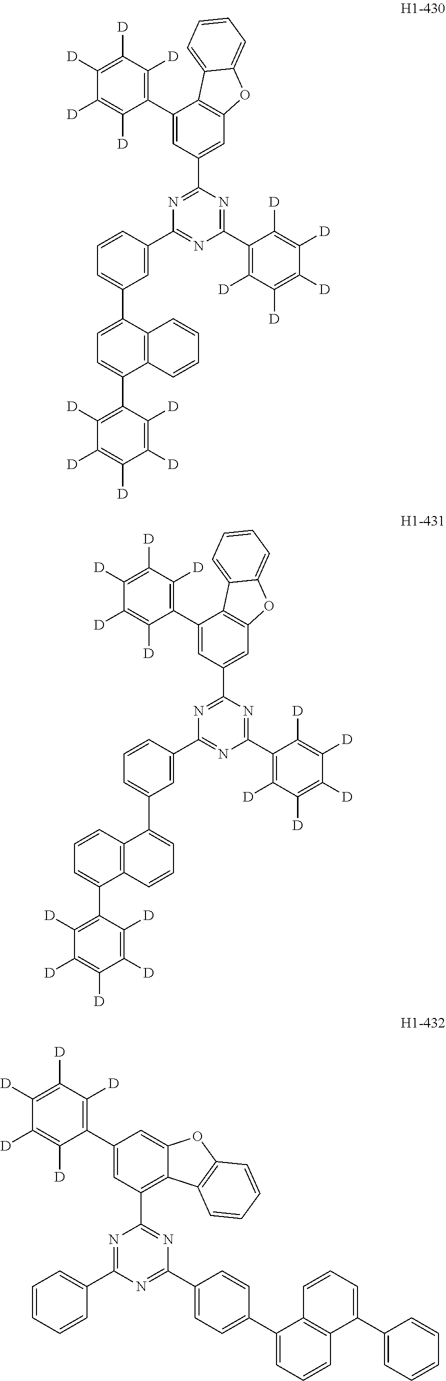 [SMILES: [2H]c1c([2H])c([2H])c(-c2nc(-c3cccc(-c4ccc(-c5c([2H])c([2H])c([2H])c([2H])c5[2H])c5ccccc45)c3)nc(-c3cc(-c4c([2H])c([2H])c([2H])c([2H])c4[2H])c4c(c3)oc3ccccc34)n2)c([2H])c1[2H] ; [2H]c1c([2H])c([2H])c(-c2nc(-c3cccc(-c4cccc5c(-c6c([2H])c([2H])c([2H])c([2H])c6[2H])cccc45)c3)nc(-c3cc(-c4c([2H])c([2H])c([2H])c([2H])c4[2H])c4c(c3)oc3ccccc34)n2)c([2H])c1[2H] ; [2H]c1c([2H])c([2H])c(-c2cc(-c3nc(-c4ccccc4)nc(-c4ccc(-c5cccc6c(-c7ccccc7)cccc56)cc4)n3)c3c(c2)oc2ccccc23)c([2H])c1[2H]]

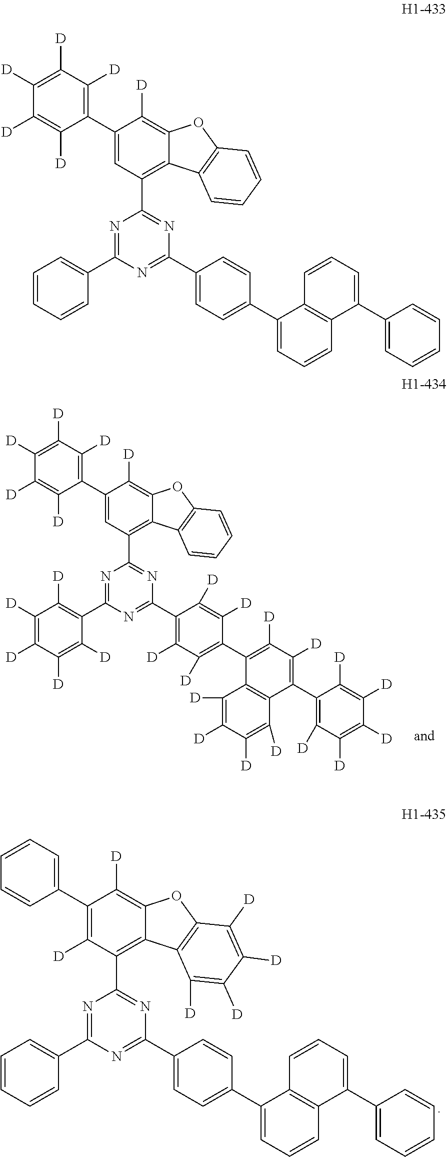 [SMILES: [2H]c1c([2H])c([2H])c(-c2cc(-c3nc(-c4ccccc4)nc(-c4ccc(-c5cccc6c(-c7ccccc7)cccc56)cc4)n3)c3c(oc4ccccc43)c2[2H])c([2H])c1[2H] ; [2H]c1c([2H])c([2H])c(-c2nc(-c3c([2H])c([2H])c(-c4c([2H])c([2H])c(-c5c([2H])c([2H])c([2H])c([2H])c5[2H])c5c([2H])c([2H])c([2H])c([2H])c45)c([2H])c3[2H])nc(-c3cc(-c4c([2H])c([2H])c([2H])c([2H])c4[2H])c([2H])c4oc5ccccc5c34)n2)c([2H])c1[2H] ; [2H]c1c([2H])c([2H])c2c(oc3c([2H])c(-c4ccccc4)c([2H])c(-c4nc(-c5ccccc5)nc(-c5ccc(-c6cccc7c(-c8ccccc8)cccc67)cc5)n4)c32)c1[2H]]

18. An organic electroluminescent material, comprising an organic electroluminescent compound according to claim 15.

19. An organic electroluminescent device, comprising an organic electroluminescent compound according to claim 15.
